(19) United States
(12) Patent Application Publication
(10) Pub. No.: US 2023/0115176 A1
(43) Pub. Date: Apr. 13, 2023

(54) Complement Modulators and Related Methods

(71) Applicant: Ra Pharmaceuticals, Inc., Cambridge, MA (US)

(72) Inventors: Willmen Wing-Menn Youngsaye, Boston, MA (US); Bolin Geng, Andover, MA (US); Michael R. Hale, Bedford, MA (US); Vincent P. Galullo, South Grafton, MA (US); Jayashree G. Tikhe, Brookline, MA (US); Timothy Ryan Siegert, Cambridge, MA (US); Alonso Ricardo, Winchester, MA (US); Derek M. LaPlaca, Somerville, MA (US); Douangsone D. Vadysirisack, Boston, MA (US); Guo-Qing Tang, Acton, MA (US); Kathleen Seyb, Wakefield, MA (US); Michelle Denise Hoarty, Billerica, MA (US); Jonathan C. Blain, Melrose, MA (US); Joseph R. Stringer, Somerville, MA (US); Yongjin Gong, Cambridge, MA (US); Claudio Sturino, Montreal (CA); Shawn Gallagher-Duval, Laval (CA); Colin Diner, Laval (CA); Burcin Akgun, Montreal (CA); Qing Cao, Belmont, MA (US); Douglas A. Treco, Arlington, MA (US); Vaishnavi Rajagopal, Andover, MA (US); Ketki Ashok Dhamnaskar, Foster City, CA (US); Zhong Ma, Lexington, MA (US); Susan Ashwell, Carlisle, MA (US); Jennifer Davoren, Cambridge, MA (US); Xiaorong Liu, Newton, MA (US); Camil Sayegh, Belmont, MA (US); Wenqing Xu, Brighton, MA (US); Alex Lemire, Laval (CA); Audrey Belouin, Montreal (CA); Alexandria Jeanneret, Laval (CA); Andrew Hildering, Montreal (CA); Barbara Bertani, Verona (IT); Fabrizio MIcheli, Verona (IT); Bemardo Pezzati, Verona (IT); Alfonso Pozzan, Verona (IT)

(21) Appl. No.: 17/442,246

(22) PCT Filed: Mar. 27, 2020
(86) PCT No.: PCT/US2020/025184

§ 371 Date: Sep. 23, 2021

Related U.S. Application Data
(60) Provisional application No. 62/826,259, filed on Mar. 29, 2019.
(60) Provisional application No. 62/826,242, filed on Mar. 29, 2019.
(60) Provisional application No. 62/826,266, filed on Mar. 29, 2019.
(60) Provisional application No. 62/826,282, filed on Mar. 29, 2019.
(60) Provisional application No. 62/864,813, filed on Jun. 21, 2019.
(60) Provisional application No. 62/864,802, filed on Jun. 21, 2019.
(60) Provisional application No. 62/988,985, filed on Mar. 13, 2020.

(51) Int. Cl. C07D 519/00; C07D 239/10; C07D 403/10; C07D 403/12; C07D 403/14; C07D 471/04

(52) U.S. Cl. CPC C07D 519/00; C07D 239/10; C07D 403/10; C07D 403/12; C07D 403/14; C07D 471/04

(57) ABSTRACT

The present disclosure presents compounds and compositions that interact with complement components. Some compounds inhibit complement activity. Included are small molecule compounds and compositions that function as C5 inhibitor compounds. Methods for inhibiting complement activity and methods of treating complement-related indications with the C5 inhibitor compounds and compositions are provided.

### CROSS REFERENCE TO RELATED APPLICATIONS

This application claims priority to U.S. Provisional Patent Application No. 62/826,242 filed Mar. 29, 2019, entitled COMPLEMENT MODULATORS AND RELATED METHODS, U.S. Provisional Patent Application No. 62/826,266 filed Mar. 29, 2019, entitled COMPLEMENT MODULATORS AND RELATED METHODS, U.S. Provisional Patent Application No. 62/826,282 filed Mar. 29, 2019, entitled COMPLEMENT MODULATORS AND RELATED METHODS, U.S. Provisional Patent Application No. 62/826,259 filed Mar. 29, 2019, entitled COMPLEMENT MODULATORS AND RELATED METHODS, U.S. Provisional Patent Application No. 62/864,813 filed Jun. 21, 2019, entitled COMPLEMENT MODULATORS AND RELATED METHODS, U.S. Provisional Patent Application No. 62/864,802 filed Jun. 21, 2019, entitled COMPLEMENT MODULATORS AND RELATED METHODS, and U.S. Provisional Patent Application No. 62/988,985 filed Mar. 13, 2020, entitled COMPLEMENT MODULATORS AND RELATED METHODS, the contents of each of which are herein incorporated by reference in their entirety.

### BACKGROUND

The vertebrate immune response is comprised of adaptive and innate immune components. While the adaptive immune response is selective for particular pathogens and is slow to respond, components of the innate immune response recognize a broad range of pathogens and respond rapidly upon infection. One such component of the innate immune response is the complement system.

The complement system includes about 20 circulating proteins, synthesized primarily by the liver. Components of this particular immune response were first termed “complement” due to the observation that they complemented the antibody response in the destruction of bacteria. These proteins remain in an inactive form prior to activation in response to infection. Activation occurs by way of a pathway of proteolytic cleavage initiated by pathogen recognition and leading to pathogen destruction. Three such pathways are known in the complement system and are referred to as the classical pathway, the lectin pathway, and the alternative pathway. The classical pathway is activated when an IgG or IgM molecule binds to the surface of a pathogen. The lectin pathway is initiated by the mannan-binding lectin protein recognizing the sugar residues of a bacterial cell wall. The alternative pathway remains active at low levels in the absence of any specific stimuli. While all three pathways differ with regard to initiating events, all three pathways converge with the cleavage of complement component C3. C3 is cleaved into two products termed C3a and C3b. Of these, C3b becomes covalently linked to the pathogen surface while C3a acts as a diffusible signal to promote inflammation and recruit circulating immune cells. Surface-associated C3b forms a complex with other components to initiate a cascade of reactions among the later components of the complement system. Due to the requirement for surface attachment, complement activity remains localized and minimizes destruction to non-target cells.

Pathogen-associated C3b facilitates pathogen destruction in two ways. In one pathway, C3b is recognized directly by phagocytic cells and leads to engulfment of the pathogen. In the second pathway, pathogen-associated C3b initiates the formation of the membrane attack complex (MAC). In the first step, C3b complexes with other complement components to form the C5-convertase complex. Depending on the initial complement activation pathway, the components of this complex can differ. C5-convertase formed as the result of the classical complement pathway comprises C4b and C2a in addition to C3b. When formed by the alternative pathway, C5-convertase comprises two subunits of C3b as well as one Bb component.

Complement component C5 is cleaved by either C5-convertase complex into C5a and C5b. C5a, much like C3a, diffuses into the circulation and promotes inflammation, acting as a chemoattractant for inflammatory cells. C5b remains attached to the cell surface where it triggers the formation of the MAC through interactions with C6, C7, C8 and C9. The MAC is a hydrophilic pore that spans the membrane and promotes the free flow of fluid into and out of the cell, thereby destroying it.

An important component of all immune activity is the ability of the immune system to distinguish between self and non-self cells. Pathologies arise when the immune system is unable to make this distinction. In the case of the complement system, vertebrate cells express proteins that protect them from the effects of the complement cascade. This ensures that targets of the complement system are limited to pathogenic cells. Many complement-related disorders and diseases are associated with abnormal destruction of self cells by the complement cascade. In one example, subjects suffering from paroxysmal nocturnal hemoglobinuria (PNH) are unable to synthesize functional versions of the complement regulatory proteins CD55 and CD59 on hematopoietic stem cells. This results in complement-mediated hemolysis and a variety of downstream complications. Other complement-related disorders and diseases include, but are not limited to: autoimmune diseases and disorders; neurological diseases and disorders; blood diseases and disorders; and infectious diseases and disorders. Experimental evidence suggests that many complement-related disorders are alleviated through inhibition of complement activity.

Organic small molecule inhibitors of complement have been developed in the past. Small molecule inhibitors have advantages as they can be delivered via many pathways, including oral and topical delivery, they are affordable and have pharmacokinetic advantages. Some of the challenges in development of small molecules have been involved with poor selectivity, weak potency, short half-life and toxicity. Therefore, there is a need for the development of small molecule compounds and compositions that overcome these challenges. The present disclosure addresses this need by presenting small molecule compounds and compositions for complement modulation and related methods of use.

### SUMMARY

In some embodiments, the present disclosure provides a compound having a structure of Formula (700):

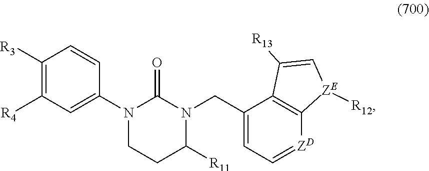

or a pharmaceutically acceptable salt thereof, wherein: R3 and R4 may be independently an alkyl, cyclic alkyl, alkenyl, alkynyl, halogen, hydroxyl, alkoxyl, ether, CN, amine, amide, aryl, or heteroaryl, wherein the alkyl, cyclic alkyl, alkenyl, alkynyl, alkoxyl, ether, amine, aryl, or heteroaryl is optionally substituted; R11 may be H or an alkyl group, wherein the alkyl group is optionally substituted; R12 may be H, an alkyl, alkenyl, alkynyl, halogen, hydroxyl, alkoxyl, ether, CN, amine, amide, aryl, heteroaryl, cyclic alkyl, heterocyclic alkyl, multicyclic alkyl group, or hetero multicyclic alkyl group, wherein the alkyl, alkenyl, alkynyl, alkoxy, ether, amine, aryl, heteroaryl group, cyclic alkyl, heterocyclic alkyl, multicyclic alkyl, or hetero multicyclic alkyl group is optionally substituted; R13 may be H, a halogen, —CN, —CF3, or a C1-C3 alkyl group; Z^{D} may be N or CR₁₄, wherein R14 is H or an alkyl group, wherein the alkyl group is optionally substituted; and Z^{E} may be N or CH. R3 may be —OCH3, R4 ay be an alkoxyl group. R4 may be

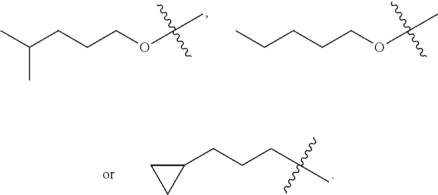

R12 may include an amide group.

In some embodiments, the present disclosure provides a compound having a structure of Formula (701):

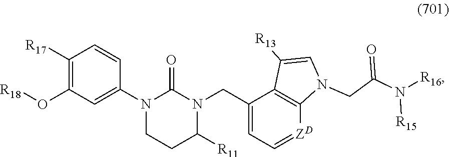

or a pharmaceutically acceptable salt thereof, wherein: R11 may be H or a methyl group; R13 may be H, halogen, —CN, —CF3, or a C1-C3 alkyl group; R15 and R16 may be independently a H, alkyl, aryl, heteroaryl, cyclic alkyl, heterocyclic alkyl, multicyclic alkyl group, or hetero multicyclic alkyl group, wherein the alkyl, aryl, heteroaryl group, cyclic alkyl, heterocyclic alkyl, multicyclic alkyl, or hetero multicyclic alkyl group is optionally substituted, wherein R15 and R16, together with the nitrogen they are attached to, optionally form a 3 to 8 membered heterocyclic group, wherein the heterocyclic group may be optionally substituted; R17 may be a halogen, an alkyl group, or an alkoxyl group; R18 may be an alkyl group; and Z^{D} may be N or CR₁₄, wherein R14 is H or an alkyl group, wherein the alkyl group is optionally substituted. R13 may be H or Cl. R17 may be —OCH3. R18 may be

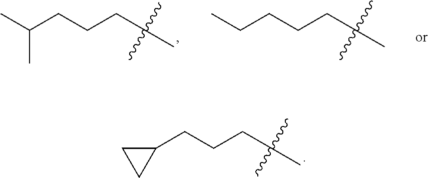

Z^{D} may be N or CH. R15 and R16 may both be C1-C3 alkyl groups. R15 and R16 may be methyl groups. R15 and R16, together with the nitrogen they are attached to, may form a 6-membered non-aromatic heterocyclic group. The 6-membered non-aromatic heterocyclic group may be

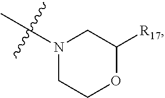

wherein R17 is an alkyl group, wherein the alkyl group is optionally substituted. R17 may be an alkyl group substituted with an amine group.

In some embodiments, the present disclosure provides a compound having a structure of Formula (IIe):

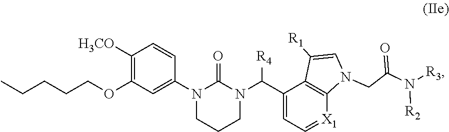

or a pharmaceutically acceptable salt thereof, wherein: X1 may be CH or N; R1 may be H, a halogen, —CN, —CF3, or a C1-C3 alkyl group, wherein the halogen is optionally selected from the group consisting of Cl, F, Br and I; R2 and R3 may be independently a H, alkyl, aryl, heteroaryl, cyclic alkyl, heterocyclic alkyl, a multicyclic alkyl group, or a hetero multicyclic alkyl group, wherein the alkyl, aryl, heteroaryl group, cyclic alkyl, heterocyclic alkyl, multicyclic alkyl, or hetero multicyclic alkyl is optionally substituted, wherein R2 and R3, together with the nitrogen they are attached, optionally form a 3 to 8 membered heterocyclic group, wherein the heterocyclic group is optionally substituted; and R4 may be H or a C1-C3 alkyl group. R2 and R3 may be both C1-C3 alkyl groups. R4 may be H. The compound may be selected from the group consisting of CU0025, CU0028, CU0029, CU0030, CU0031, CU0035, CU0043, CU0046, CU0048, CU0049, CU0050, CU0051, CU0053, CU0056, CU0057, CU0060, CU0062, CU0231, CU0232, CU0235, CU0239, CU0243, CU0244, CU0245, CU0246, CU0247, CU0255, CU0257, CU0258, CU0260, CU0261, CU0504, CU0506, CU0508, CU0509, CU0510, CU0518, CU0519, CU0521, CU0526, CU0528, CU0529, CU0533, CU0534, CU0535, CU0538, CU0539, CU0540, CU0541, CU0543, CU0549, CU0553, CU0560, CU0561, CU0567, CU0602, CU0603, CU0747, and CU0817.

In some embodiments, the present disclosure provides a compound having a structure of Formula (f):

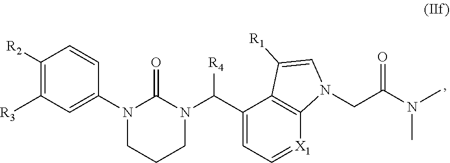

or a pharmaceutically acceptable salt thereof, wherein: X1 may be CH or N; R1 may be H, a halogen, —CN, —CF3, or a C1-C3 alkyl group, wherein the halogen is optionally selected from the group consisting of Cl, F, Br and I; R2 and R3 may be independently an alkyl, cyclic alkyl, alkenyl, alkynyl, halogen, hydroxyl, alkoxyl, ether, CN, amine, amide, aryl, or heteroaryl, wherein the alkyl, cyclic alkyl, alkenyl, alkynyl, alkoxyl, ether, amine, aryl, or heteroaryl is optionally substituted; R4 may be H or a C1-C3 alkyl group. R2 and R3 may both be alkoxyl groups. R2 may be —OCH3. R4 may be H. The compound may be selected from the group consisting of CU0025, CU0026, CU0027, CU0035, CU0036, CU0231, CU0232, CU0252, CU0253, CU0256, CU0258, CU0259, CU0261, CU0262, CU0508, CU0515, CU0516, CU0532, CU0535, CU0543, CU0582, CU0591, CU0595, CU0602, CU0606, CU0610, CU0625, CU0681, CU0707, CU0737, CU0747, CU0752, CU0761, CU0764, CU0765, CU0767, CU0780, CU0790, CU0799, CU0800, CU0803, CU0811, CU0828, CU0843, CU0846, and CU0847.

In some embodiments, the present disclosure provides a compound having a structure of Formula (IId1):

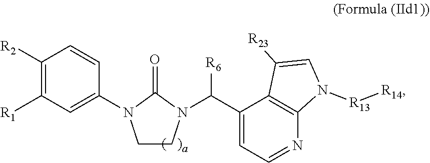

or a pharmaceutically acceptable salt thereof wherein: “a” may be 1, 2 or 3; R₁ may be a C₁-C₇ alkyl group or a C₁-C₇ alkoxy group, wherein R₁ is optionally substituted with one or more substituents selected from the group consisting of an alkyl, an alkoxyl, a halogen, a phenyl group, a cyclic group, a bicyclic group, an alkenyl group, and an alkynyl group, wherein each of the one or more substituents is optionally further substituted with at least one halogen, alkyl group, or alkoxyl group; R₂ may be a branched or linear C1-C4 alkoxy group or a C₃-C₅ cycloalkyl group; Rf may be hydrogen, OH, a C₁-C₃ alkyl group, or a C₁-C₃ alkoxyl group; R₁₃ may be a bond, a C₁-C₃ alkyl group, a group that includes a carbonyl group, a cyclic group, or heterocyclic group; R₁₄ may be hydrogen, a pyridine optionally substituted with one or more C₁-C₄ alkyl groups, an amine group optionally substituted with one or two alkyl groups, a cyclic or heterocyclic group optionally substituted with one or more alkyl groups, a functional group including a carbonyl group (—CO—), an amide group (—CO—NH—) optionally substituted with one alkyl group, —CH═N— optionally substituted with an alkyl group or an amine group, a pyrrolidinone optionally substituted with one or more C₁-C₄ alkyl groups, a triazole optionally substituted with a C₁-C₄ alkyl group, —CO—N(R₁₅)₂, —CO—R₁₆,

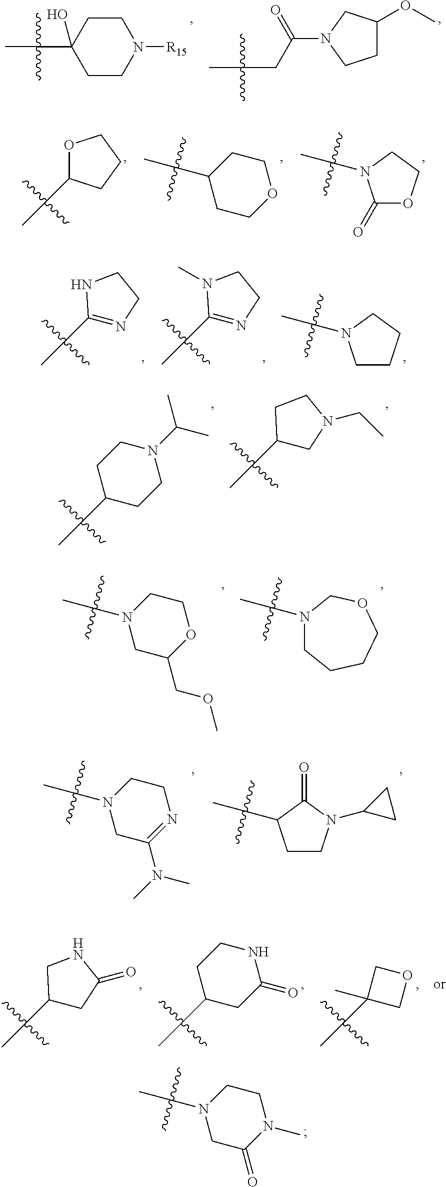

each R₁₅ may be hydrogen or a C₁-C₄ alkyl group; R₁₆ may be a morpholine, a piperazine, or
an oxazepane; wherein each R₁₆ is optionally substituted with one or more substituents selected from the group consisting of a C₁-C₄ alkyl group, a C₃-C₅ cycloalkyl group, a C₁-C₃ hydroxyalkyl group, a C₁-C₄ alkoxy group, a C₁-C₄ alkylmethoxy group, a C₁-C₄ alkylethoxy group, —(C₁-C₃ alkyl)-N(R₁₅)₂, a C₁-C₃ alkylpyrrolidine group, an acetyl group, and an oxo group; R₁₇ may be hydrogen or a C₁-C₄ alkyl group; and R₂₃ may be hydrogen, an alkyl, or a halogen. The compound may be selected from the group consisting of CU0032, CU0033, CU0034, CU0035, CU0036, CU0037, CU0038, CU0039, CU0040, CU0041, CU0042, CU0043, CU0044, CU0045, CU0046, CU0047, CU0048, CU0049, CU0050, CU0051, CU0052, CU0053, CU0054, CU0055, CU0056, CU0057, CU0058, CU0059, CU0060, CU0061, CU0062, CU0248, CU0249, CU0250, CU0251, CU0252, CU0253, CU0254, CU0255, CU0256, CU0257, CU0258, CU0259, CU0260, CU0261 and CU0262.

In some embodiments, the present disclosure provides a compound having a structure selected from the group consisting of SM0001, SM0002, SM0003, SM0004, SM0005, SM0006, SM0007, SM0008, SM0009, SM0010, SM0011, SM0012, SM0013, SM0014, SM0015, SM0016, SM0017, SM0018, SM0019, SM0020, SM0021, SM0022, SM0023, SM0024, SM0025, SM0026, SM0027, SM0028, SM0029, SM0030, SM0031, SM0032, SM0033, SM0034, SM0035, SM0036, SM0037, SM0038, SM0039, SM0040, SM0041, SM0042, SM0043, SM0044, SM0045, SM0046, SM0047, SM0048, SM0049, SM0050, SM0051, SM0052, SM0053, SM0054, SM0055, SM0056, SM0057, SM0058, SM0059, SM0060, SM0061, SM0062, SM0063, SM0064, SM0065, SM0066, SM0067, SM0068, SM0069, SM0070, SM0071, SM0072, SM0073, SM0074, SM0075, SM0076, SM0077, SM0078, SM0079, SM0080, SM0081, SM0082, SM0083, SM0084, SM0085, SM0086, SM0087, SM0088, SM0089, SM0090, SM0091, SM0092, SM0093, SM0094, SM0095, SM0096, SM0097, SM0098, SM0099, SM0100, SM0101, SM0102, SM0103, SM0104, SM0105, SM0106, SM0107, SM0108, SM0109, SM0110, SM0111, SM0112, SM0113, SM0114, SM0115, SM0116, SM0117, SM0118, SM0119, SM0120, SM0121, SM0200, SM0201, SM0202, SM0203, SM0204, SM0205, SM0206, SM0207, SM0208, SM0209, SM0210, SM0211, SM0212, SM0213, SM0214, SM0215, SM0216, SM0217, SM0218, SM0219, C5INH-0294, C5INH-0296, C5INH-0298, C5INH-0303, C5INH-0310, C5INH-0311, C5INH-0315, C5INH-0316, C5INH-0317, C5INH-0318, C5INH-0319, C5INH-0321, C5INH-0323, C5INH-0324, C5INH-0326, C5INH-0329, C5INH-0330, C5INH-0333, C5INH-0335, C5INH-0336, C5INH-0338, C5INH-0339, C5INH-0340, C5INH-0342, C5INH-0343, C5INH-0348, C5INH-0349, C5INH-0350, C5INH-0352, C5INH-0353, C5INH-0355, C5INH-0356, C5INH-0357, C5INH-0361, C5INH-0366, C5INH-0367, C5INH-0369, C5INH-0370, C5INH-0371, C5INH-0372, C5INH-0373, C5INH-0377, C5INH-0379, C5INH-0381, C5INH-0382, C5INH-0383, C5INH-0384, C5INH-0385, C5INH-0387, C5INH-0388, C5INH-0389, C5INH-0390, C5INH-0391, C5INH-0395, C5INH-0396, C5INH-0397, C5INH-0398, C5INH-0399, C5INH-0401, C5INH-0402, C5INH-0403, C5INH-0406, C5INH-0409, C5INH-0410, C5INH-0411, C5INH-0414, C5INH-0417, C5INH-0420, C5INH-0421, C5INH-0422, C5INH-0425, C5INH-0428, C5INH-0431, C5INH-0432, C5INH-0436, C5INH-0437, C5INH-0438, C5INH-0440, C5INH-0443, C5INH-0446, C5INH-0447, C5INH-0448, C5INH-0450, C5INH-0452, C5INH-0453, C5INH-0454, C5INH-0456, C5INH-0458, C5INH-0460, C5INH-0462, C5INH-0463, C5INH-0469, C5INH-0472, C5INH-0473, C5INH-0474, C5INH-0476, C5INH-0477, C5INH-0484, C5INH-0485, C5INH-0486, C5INH-0487, C5INH-0488, C5INH-0489, C5INH-0490, C5INH-0491, C5INH-0492, C5INH-0496, C5INH-0497, C5INH-0498, C5INH-0500, C5INH-0501, C5INH-0502, C5INH-0504, C5INH-0507, C5INH-0508, C5INH-0509, C5INH-0510, C5INH-0512, C5INH-0513, C5INH-0515, C5INH-0516, C5INH-0517, C5INH-0518, C5INH-0519, C5INH-0521, C5INH-0524, C5INH-0525, C5INH-0526, C5INH-0527, C5INH-0532, C5INH-0533, C5INH-0534, C5INH-0535, C5INH-0536, C5INH-0537, C5INH-0538, C5INH-0539, C5INH-0540, C5INH-0541, C5INH-0543, C5INH-0544, C5INH-0545, C5INH-0547, CU0001, CU0002, CU0003, CU0004, CU0005, CU0006, CU0007, CU0008, CU0009, CU0010, CU0011, CU0012, CU0013, CU0014, CU0015, CU0016, CU0017, CU0018, CU0019, CU0020, CU0021, CU0022, CU0023, CU0024, CU0025, CU0026, CU0027, CU0028, CU0029, CU0030, CU0031, CU0032, CU0033, CU0034, CU0035, CU0036, CU0037, CU0038, CU0039, CU0040, CU0041, CU0042, CU0043, CU0044, CU0045, CU0046, CU0047, CU0048, CU0049, CU0050, CU0051, CU0052, CU0053, CU0054, CU0055, CU0056, CU0057, CU0058, CU0059, CU0060, CU0061, CU0062, CU0063, CU0064, CU0065, CU0066, CU0067, CU0100, CU0101, CU0102, CU0103, CU0104, CU0105, CU0106, CU0107, CU0108, CU0109, CU0110, CU0111, CU0112, CU0113, CU0114, CU0115, CU0116, CU0117, CU0118, CU0119, CU0120, CU0121, CU0122, CU0123, CU0124, CU0125, CU0126, CU0127, CU0128, CU0129, CU0130, CU0131, CU0132, CU0133, CU0134, CU0135, CU0136, CU0137, CU0138, CU0139, CU0140, CU0141, CU0142, CU0143, CU0144, CU0145, CU0146, CU0147, CU0148, CU0149, CU0150, CU0151, CU0152, CU0153, CU0154, CU0155, CU0156, CU0157, CU0158, CU0159, CU0160, CU0161, CU0162, CU0163, CU0164, CU0165, CU0166, CU0167, CU0168, CU0169, CU0170, CU0171, CU0172, CU0173, CU0174, CU0175, CU0176, CU0177, CU0178, CU0179, CU0180, CU0181, CU0182, CU0183, CU0184, CU0185, CU0186, CU0187, CU0188, CU0189, CU0190, CU0191, CU0192, CU0193, CU0194, CU0195, CU0196, CU0197, CU0198, CU0199, CU0200, CU0201, CU0202, CU0203, CU0204, CU0205, CU0206, CU0207, CU0208, CU0209, CU0210, CU0211, CU0212, CU0213, CU0214, CUO215, CU0216, CU0217, CU0218, CU0219, CU0220, CU0221, CU0222, CU0223, CU0224, CU0225, CU0226, CU0227, CU0228, CU0229, CU0230, CU0231, CU0232, CU0233, CU0234, CU0235, CU0236, CU0237, CU0238, CU0239, CU0240, CU0241, CU0242, CU0243, CU0244, CU0245, CU0246, CU0247, CU0248, CU0249, CU0250, CU0251, CU0252, CU0253, CU0254, CU0255, CU0256, CU0257, CU0258, CU0259, CU0260, CU0261, CU0262, CU0500, CU0501, CU0502, CU0503, CU0504, CU0505, CU0506, CU0507, CU0508, CU0509, CU0510, CU0511, CU0512, CU0513, CU0514, CU0515, CU0516, CU0517, CU0518, CU0519, CU0520, CU0521, CU0522, CU0523, CU0524, CU0525, CU0526, CU0527, CU0528, CU0529, CU0530, CU0531, CU0532, CU0533, CU0534, CU0535, CU0536, CU0537, CU0538, CU0539, CU0540, CU0541, CU0542, CU0543, CU0544, CU0545, CU0546, CU0547, CU0548, CU0549, CU0550, CU0551, CU0552, CU0553, CU0554, CU0555, CU0556, CU0557, CU0558, CU0559, CU0560, CU0561, CU0562, CU0563, CU0564, CU0565, CU0566, CU0567, CU0568, CU0569, CU0570, CU0571, CU0572, CU0573, CU0574, CU0575, CU0576, CU0577, CU0578, CU0579, CU0580, CU0581, CU0582, CU0583, CU0584, CU0585, CU0586, CU0587, CU0588, CU0589, CU0590, CU0591, CU0592, CU0593, CU0594, CU0595, CU0596, CU0597, CU0598, CU0599, CU0600, CU0601, CU0602, CU0603, CU0604, CU0605, CU0606, CU0607, CU0608, CU0609, CU0610, CU0611, CU0612, CU0613, CU0614, CU0615, CU0616, CU0617, CU0618, CU0619, CU0620, CU0621, CU0622, CU0624, CU0625, CU0626, CU0627, CU0628, CU0629, CU0630, CU0631, CU0632, CU0633, CU0634, CU0635, CU0636, CU0637, CU0638, CU0639, CU0640, CU0641, CU0642, CU0643, CU0644, CU0645, CU0646, CU0647, CU0648, CU0649, CU0650, CU0651, CU0652, CU0653, CU0654, CU0655, CU0656, CU0657, CU0658, CU0659, CU0660, CU0661, CU0662, CU0663, CU0664, CU0665, CU0666, CU0667, CU0668, CU0669, CU0670, CU0671, CU0672, CU0673, CU0674, CU0675, CU0676, CU0677, CU0678, CU0679, CU0680, CU0681, CU0682, CU0683, CU0684, CU0685, CU0686, CU0687, CU0688, CU0689, CU0690, CU0691, CU0692, CU0693, CU0694, CU0695, CU0696, CU0697, CU0698, CU0699, CU0700, CU0701, CU0702, CU0703, CU0704, CU0705, CU0706, CU0707, CU0708, CU0709, CU0710, CU0711, CU0712, CU0713, CU0714, CU0715, CU0716, CU0717, CU0718, CU0719, CU0720, CU0721, CU0722, CU0723, CU0724, CU0725, CU0726, CU0727, CU0728, CU0729, CU0730, CU0731, CU0732, CU0733, CU0734, CU0735, CU0736, CU0737, CU0738, CU0739, CU0740, CU0741, CU0742, CU0743, CU0744, CU0745, CU0746, CU0747, CU0748, CU0749, CU0750, CU0751, CU0752, CU0753, CU0754, CU0755, CU0756, CU0757, CU0758, CU0759, CU0760, CU0761, CU0762, CU0763, CU0764, CU0765, CU0766, CU0767, CU0768, CU0769, CU0770, CU0771, CU0772, CU0773, CU0774, CU0775, CU0776, CU0777, CU0778, CU0779, CU0780, CU0781, CU0782, CU0783, CU0784, CU0785, CU0786, CU0787, CU0788, CU0789, CU0790, CU0791, CU0792, CU0793, CU0794, CU0795, CU0796, CU0797, CU0798, CU0799, CU0800, CU0801, CU0802, CU0803, CU0804, CU0805, CU0806, CU0807, CU0808, CU0809, CU0810, CU0811, CU0812, CU0813, CU0814, CU0815, CU0816, CU0817, CU0818, CU0819, CU0820, CU0821, CU0822, CU0823, CU0824, CU0825, CU0826, CU0827, CU0828, CU0829, CU0830, CU0831, CU0832, CU0833, CU0834, CU0835, CU0836, CU0837, CU0838, CU0839, CU0840, CU0841, CU0842, CU0843, CU0844, CU0845, CU0846, CU0847, SC0001, SC0002, SC0003, SC0004, SC0005, SC0006, SC0007, SC0008, SC0009, SC0010, CU0623, SC0011, SC0012, SC0013, SC0014, SC0015, SC0016, SC0017, SC0018, SC0019, SC0020, SC0021, SC0022, SC0023, SC0024, SC0025, SC0026, SC0027, SC0028, SC0029, SC0030, SC0031, SC0032, SC0033, SC0034, SC0035, SC0036, SC0037, SC0038, SC0039, SC0040, SC0041, SC0042, SC0043, SC0044, SC0045, SC0046, SC0047, SC0048, SC0049, SC0050, SC0051, SC0052, SC0053, SC0054, SC0055, SC0056, SC0057, SC0058, SC0059, SC0060, SC0061, SC0062, SC0063, SC0064, SC0065, SC0066, SC0067, SC0068, SC0069, SC0070, SC0071, SC0072, SC0100, SC0101, SC0102, SC0103, SC0104, SC0105, SC0106, SC0107, SC0108, SC0109, SC0110, SC0111, SC0112, SC0113, SC0114, SC0115, SC0116, SC0117, SC0118, SC0119, SC0120, SC0121, SC0122, SC0123, SC0124, SC0125, SC0126, SC0127, SC0128, SC0129, SC0130, SC0131, SC0132, SC0133, SC0134, SC0135, SC0136, SC0137, SC0138, SC0139, SC0140, SC0141, SC0142, SC0143, SC0144, SC0145, SC0146, SC0147, SC0148, SC0149, SC0150, SC0151, SC0152, SC0153, SC0154, SC0155, SC0156, SC0157, SC0158, SC0159, SC0160, SC016, SC0162, SC0163, SC0164, SC0165, SC0166, SC0167, SC0168, SC0169, SC0170, SC0171, SC0172, SC0173, SC0174, SC0175, SC0176, SC0177, SC0178, SC0179, SC0180, SC0181, SC0182, SC0183, SC0184, SC0185, SC0186, SC0187, SC0188, SC0189, SC0190, SC0191, SC0192, SC0193, SC0194, SC0195, SC0196, SC0197, SC0198, SC0199, SC0200, SC0201, SC0202, SC0203, SC0204, SC0205, SC0206, SC0207, SC0208, SC0209, SC0210, SC0211, SC0212, SC0213, SC0214, SC0215, SC0216, SC0217, SC0218, SC0219, SC0220, SC0221, SC0222, SC0223, SC0224, SC0225, SC0226, SC0227, SC0228, SC0229, SC0230, SC0231, and SC0232.

In some embodiments, the present disclosure provides a pharmaceutical composition that includes any of the compounds described herein or a pharmaceutically acceptable salt thereof, and a pharmaceutically acceptable excipient. The pharmaceutical composition may be formulated for oral delivery. The pharmaceutical composition may have a format selected from the group consisting of a liquid, a tablet, a pill, and a capsule.

In some embodiments, the present disclosure provides a method of inhibiting complement activity in a biological system by contacting the biological system with a C5 inhibitor, wherein the C5 inhibitor includes any of the compounds described here or a pharmaceutically acceptable salt thereof. The C5 inhibitor may have an equilibrium dissociation constant (K_{D}) for association with C5 of from about 0.01 nM to about 10,000 nM. The C5 inhibitor may inhibit red blood cell lysis with a half maximal inhibitory concentration (IC50) of from about 0.01 nM to about 5,000 nM. The biological system may include one or more of a cell, a tissue, an organ, and a bodily fluid. The biological system may include a subject, wherein the subject is a mammal. The subject may be a human.

In some embodiments, the present disclosure provides a method of inhibiting complement activity in a subject by administering a compound or pharmaceutical composition disclosed herein to the subject. The complement activity may include C5 activity.

In some embodiments, the present disclosure provides a method of treating a complement-related indication in a subject by administering a compound or pharmaceutical composition disclosed herein to the subject. The complement-related indication may be selected from the group consisting of paroxysmal nocturnal hemoglobinuria, an inflammatory indication, a wound, an injury, an autoimmune indication, a pulmonary indication, a cardiovascular indication, a neurological indication, a kidney-related indication, an ocular indication, and a pregnancy-related indication. The administration may include intravenous, subcutaneous, oral, or topical administration. The subject may be resistant to treatment with eculizumab. The subject may have previously been treated with eculizumab.

In some embodiments, the present disclosure provides a C5-interacting compound, wherein the C5-interacting compound binds to C5 and includes a compound disclosed herein. The C5-interacting compound may bind to at least one cysteine residue of C5. The C5-interacting compound may inhibit C5 cleavage. The C5-interacting compound may exhibit a kinetic solubility value of from about 10 μM to about 500 μM, wherein the kinetic solubility value is determined for solubility in 0.5 M phosphate buffered saline, pH 7.4. The kinetic solubility value may be from about 20 μM to about 50 μM. The C5-interacting compound may exhibit an apparent permeability (Pₐₚₚ) value for movement across a cell monolayer of from about 0.1×10⁻⁶ cm/s to about 30×10⁻⁶ cm/s, wherein the Pₐₚₚ value is determined by measuring apical to basolateral movement across a Madin Darby canine kidney (MDCK) cell monolayer. The C5-interacting compound may exhibit an efflux ratio of from about 5 to about 150, wherein the efflux ratio is determined by obtaining a Pₐₚₚ value for apical to basolateral movement (Pₐₚₚ A-B) across the MDCK cell monolayer; obtaining a Pₐₚₚ value for basolateral to apical movement (Pₐₚₚ B-A) across the MDCK cell monolayer; and calculating the ratio of Pₐₚₚ A-B to Pₐₚₚ B-A.

### DESCRIPTION OF THE FIGURES

The foregoing and other objects, features and advantages of particular embodiments of the disclosure will be apparent from the following description and illustrations in the accompanying figures.

FIG. 1A is a graph showing flow cytometry counts associated with fluorescently labeled CD59 in an untreated control sample including CD59-deficient cells from PNH patients and donor matched serum.

FIG. 1B is a graph showing flow cytometry counts associated with fluorescently labeled CD59 in a control sample including CD59-deficient cells from PNH patients and donor matched serum acidified with HCl to induce hemolysis.

FIG. 1C is a graph showing flow cytometry counts associated with fluorescently labeled CD59 in a sample including CD59-deficient cells from PNH patients, donor matched serum acidified with HCl to induce hemolysis, and eculizumab inhibitor.

FIG. 1D is a graph showing flow cytometry counts associated with fluorescently labeled CD59 in a sample including CD59-deficient cells from PNH patients, donor matched serum acidified with HCl to induce hemolysis, and SM0001 inhibitor.

FIG. 2 is a picture of wells from an assay plate, wherein the wells include CD59-deficient cells from PNH patients, donor matched serum acidified with HCl to induce hemolysis, and increasing concentrations of SM0001 inhibitor. In the assay, darker well coloring is indicative of increased hemolysis.

### DETAILED DESCRIPTION

The present disclosure provides compounds and compositions for modulating complement and addressing complement-related indications. Such compounds and compositions may include compounds that interact with complement components, referred to herein as “complement-interacting compounds.” Complement-interacting compounds may bind complement components and/or modulate complement activity. As used herein, “complement activity” includes the activation of the complement cascade, the formation of cleavage products from a complement component (e.g., C3 or C5), the assembly of downstream complexes following a cleavage event, or any process or event attendant to, or resulting from, the cleavage of a complement component, e.g., C3 or C5. Complement-interacting compounds may include chemical compounds, for example, small molecules or pharmaceutically acceptable salt forms of the small molecules that are capable of interacting with complement components. Some compounds may inhibit complement activation.

As used herein, “complement component C5” or “C5” is a protein complex which is cleaved by C5 convertase into at least the cleavage products, C5a and C5b. In some embodiments, complement-interacting compounds of the present disclosure may associate with C5, cleavage products of C5, and/or modulate C5 activity. These compounds are referred to herein as “C5-interacting compounds.” C5-interacting compounds that inhibit complement activation at the level of complement component C5 are referred to herein as “C5 inhibitor compounds” or “C5 inhibitors.” Some C5 inhibitors function by preventing the cleavage of C5 to the cleavage products C5a and C5b. Such inhibitors may also be referred to herein as “C5 cleavage inhibitors.”

In some embodiments, C5 inhibitors may inhibit C5 cleavage in a system. As used herein, a “system” refers to a group of related parts that function together. Such systems include those comprising C5, referred to here as “C5 systems.” C5 systems may include, but are not limited to solutions, matrices, cells, tissues, organs, and bodily fluids (including, but not limited to blood). In some cases, C5 systems may be biological systems. As used herein the term “biological system” refers to a cell, a group of cells, a tissue, an organ, a group of organs, an organelle, a biological signaling pathway (e.g., a receptor-activated signaling pathway, a charge-activated signaling pathway, a metabolic pathway, a cellular signaling pathway, etc.), a group of proteins, a group of nucleic acids, or a group of molecules (including, but not limited to biomolecules) that carry out at least one biological function or biological task within cellular membranes, cellular compartments, cells, cell cultures, tissues, organs, organ systems, organisms, multicellular organisms, or any biological entities. In some embodiments, biological systems are “cellular systems.” As used herein, “cellular system” refers to a biological system that includes one or more cells or one or more components or products of a cell. In some cases, C5 systems may include in vivo systems, in vitro systems, and/or ex vivo systems. In vivo C5 systems may include or be included in a subject.

C5 inhibitor compounds may include suitable reacting groups for reacting with functional groups on a protein. The reacting group may possess C5 inhibiting and/or interacting properties.

In C5 systems, C5 and other system components may be in solution or may be fixed, e.g., to a solid support, such as in an assay well. C5 systems may further include other components of complement, in some cases including all of the components necessary to form the membrane attack complex (MAC). In some embodiments, C5 inhibitors of the present disclosure may be used to inhibit C5 cleavage in a human subject. Such compounds may find utility in treating various complement-related indications, as described herein.

Cleavage of C5 yields the proteolytic products C5a and C5b. The cleavage site of C5 that is cleaved to yield these products is referred to herein as the C5a-C5b cleavage site. As used herein when referring to polypeptides the term “site” may be used to refer to any position within a polypeptide. Sites include locations on a polypeptide that may be modified, manipulated, altered, derivatized, or varied in response to one or more factors or stimuli. A “cleavage site,” as it pertains to amino acid based embodiments, refers to a location between two amino acid residues where a polypeptide may be divided after disruption of the adjoining peptide bond.

C5b contributes to the formation of the membrane attack complex (MAC) while C5a stimulates the immune system and the inflammatory response. In some embodiments, compounds of the present disclosure prevent the cleavage of C5 and therefore may be useful in the treatment of inflammation through the inhibition of inflammatory events including, but not limited to chemotaxis and activation of inflammatory cells (e.g. macrophages, mast cells, neutrophils and platelets), proliferation of endothelial cells and edema.

Many of the components of the complement system, including but not limited to C3, C4, and C5, are functionally inert in their native state until targeted for cleavage into multiple active components. Cleavage of C3 or C4 causes a conformational change that exposes an internal thioester domain. As used herein, the term “domain,” when referring to proteins, refers to a motif of a polypeptide having one or more identifiable structural (such as secondary or tertiary structures) or functional characteristics or properties (e.g., binding capacity, serving as a site for protein-protein interactions). Within the domain, an internal thioester linkage between cysteine and glutamine residue side chains is a chemically labile bond that confers the ability of C3 and C4 to bind cell surface and/or biological molecules. The cleavage of C3 and C4 also provides the components of the C5 convertase, either C3bC4bC2a or (C3b)2Bb. (Law, S. K., et al. (1997). Protein Science. 6:263-274; van den Elsen, J. M. H., (2002). J. Mol. Biol. 322:1103-1115; the contents of each of which are herein incorporated by reference in their entireties).

The multiple domain structure of C5 is similar to C3 and C4. The C5 convertase cleaves C5 into the components C5a and C5b. The cleavage of C5 causes a conformational change that exposes the C5b thioester-like domain, which plays a role in C5 binding C6, followed by interactions with C7 and C8 to form the cytolytic MAC. The domain structures of C5 comprise regulatory features that are critical for the processing and downstream activity of complement. (Fredslund, F. et al. (2008). Nature. 9:753-760; Hadders, M. A. et al. (2012). Cell Reports. 1:200-207).

Recently, a new paradigm for complement activation was proposed, based upon the discovery that thrombin generates previously unidentified C5 products that support the terminal complement activation pathway (Krisinger, et al., (2014). Blood. 120(8):1717-1725).

Thrombin acts in the coagulation cascade, a second circulation-based process by which organisms, in response to injury, are able to limit bleeding, restore vascular integrity, and promote healing. Subsequent to vessel damage, tissue factor (TF) is exposed to the circulation, setting off a cascade of proteolytic reactions that leads to the generation of the central coagulation enzyme thrombin, which converts fibrinogen into a fibrin clot.

Historically, the complement activation pathway has been viewed separately from the coagulation cascade; however, the interplay of these two systems is worthy of renewed consideration. Coagulation and complement are coordinately activated in an overlapping spatiotemporal manner in response to common pathophysiologic stimuli to maintain homeostasis, and disease emerges when there is unchecked activation of the innate immune and coagulation responses, as evidenced by, for example, atherosclerosis, stroke, coronary heart disease, diabetes, ischemia-reperfusion injury, trauma, paroxysmal nocturnal hemoglobinuria, age-related macular degeneration, and atypical hemolytic-uremic syndrome. In fact, introduction of complement inhibitors has been found to simultaneously treat the inflammatory and thrombotic disturbances associated with some of these disorders.

As noted above, the complement system is activated via three main pathways, all converging with proteolytic activation of the central complement component C3. Subsequently, the formation of C5 convertases results in cleavage of C5 at arginine 751 (R751) to liberate a chemotactic and anaphylatoxic C5a fragment and generate C5b. C5b is the initiating factor for assembly of the C5b dependent lytic membrane attack complex (MAC; also known as C5b-9), responsible for destroying damaged cells and pathogens.

Several molecular links between complement and coagulation have been identified. Most notably in what was described as a new complement activation pathway, thrombin was found to be capable of directly promoting activation of complement by cleaving C5, presumably at R751, thereby releasing C5a in the absence of C3 (Huber-Lang, et al., 2006. Nature Med. 12(6):682-687). However, these studies did not compare thrombin with the bona fide C5 convertase, and only limited biochemical analyses were performed; thus, the physiologic relevance of the pathway was not evaluable.

Using purified and plasma-based systems, the effects of thrombin and C5 convertase on C5 were assessed by measuring release of the anaphylatoxin C5a and generation of the C5b, component of MAC. It was discovered that, while thrombin cleaved C5 poorly at R751, yielding minimal C5a and C5b, it efficiently cleaved C5 at a newly identified, highly conserved R947 site, generating previously undescribed intermediates CST and CSbT. Tissue factor-induced clotting of plasma led to proteolysis of C5 at a thrombin-sensitive site corresponding to this new R947 site, instead of R751. Combined treatment of C5 with thrombin and C5 convertase yielded C5a and C5b_{T}, the latter forming a C5b_{T}-9 membrane attack complex with significantly more lytic activity than with C5b-9. Thus, a new paradigm has been proposed for complement activation, in which thrombin is an invariant and critical partner with C5 convertase in initiating formation of a more active MAC via formation of previously unidentified C5 products that are generated via cooperative proteolysis by the two enzymes. These discoveries provide new insights into the regulation of innate immunity in the context of coagulation activation occurring in many diseases. (Krisinger, et al., (2014). Blood. 120(8):1717-1725).

In some embodiments, compounds of the present disclosure may inhibit thrombin-induced complement activation. Such compounds may therefore be used to treat hemolysis resulting from thrombin-induced complement activation.

Given the findings of molecular links between the complement and coagulation pathways, it is believed that complement may be activated by additional components of the coagulation and/or inflammation cascades. For example, other serine proteases with slightly different substrate specificity may act in a similar way. Huber-Lang et al. (2006) showed that thrombin not only cleaved C5 but also in vitro-generated C3a when incubated with native C3 (Huber-Lang, et al., 2006. Nature Med. 12(6):682-687; the contents of which are herein incorporated by reference in their entirety). Similarly, other components of the coagulation pathway, such as FXa, FXIa and plasmin, have been found to cleave both C5 and C3.

Specifically, in a mechanism similar to the one observed via thrombin activation, it has been observed that plasmin, FXa, FIXa and FXIa are able to cleave C5 to generate C5a and C5b (Amara, et al., (2010). J. Immunol. 185:5628-5636; Amara, et al., (2008) “Interaction Between the Coagulation and Complement System” in Current Topics in Complement II, J. D. Lambris (ed.), pp. 71-79). The anaphylatoxins produced were found to be biologically active as shown by a dose-dependent chemotactic response of neutrophils and HMC-1 cells, respectively. Plasmin-induced cleavage activity could be dose-dependently blocked by the serine protease inhibitor aprotinin and leupeptine. These findings suggest that various serine proteases belonging to the coagulation system are able to activate the complement cascade independently of the established pathways. Moreover, functional C5a and C3a are generated (as detected by immunoblotting and ELISA), both of which are known to be crucially involved in the inflammatory response.

In some embodiments, compounds of the present disclosure may inhibit activation of C5 by plasmin, FXa, FIXa, FXIa and other proteases of the coagulation pathway.

In some embodiments, C5 inhibitors inhibit cleavage of C5 to C5a and C5b fragments. Analysis and detection of such inhibitory activity may be carried out by immunological assays (e.g., ELISAs). In some cases, immunological assays for detecting C5 inhibitor activity may include ELISAs detecting C5 fragments (e.g. C5a fragments). In some cases, immunological assays may detect indicators of MAC assembly.

Human leukocyte elastase (HLE), an enzyme secreted by neutrophils and macrophages during inflammatory processes, has long been known to also release from C5 a chemotactic, C5a-like fragment. However, this C5a-like fragment, is not identical with C5a, as HLE does not cleave peptide bonds at the cleavage site that ordinarily cleaves C5 into C5a and C5b after the exposure to the complement convertases. Rather, cleavage of complement C5 by HLE has also been found to generate a functionally active C5b-like molecule that is able to participate in MAC formation (Vogt, (1999). Immunobiology. 201:470-477).

In some embodiments, compounds of the present disclosure may inhibit activation of C5 by HLE and other proteases of the inflammation cascade.

### I. Compounds and Compositions

### C5-Interacting Compounds

In some embodiments, C5-interacting compounds of the present disclosure may be small molecules. Such small molecule compounds may have a size of from about 100 to about 20000 g/mol (e.g. from about 100 to about 200, to about 300, to about 400, to about 500, to about 600, to about 700, to about 800, to about 900, to about 1000, to about 1100, to about 1200, to about 1300, to about 1400, to about 1500, to about 1600, to about 1700, to about 1800, to about 1900, to about 2000, to about 5000, to about 10000, to about 15000, or to about 20000 g/mol). In some embodiments, compounds may have a size of from about 200 to about 1000 g/mol.

C5-interacting compounds of the present disclosure may have a topological polar surface area (TPSA) of from about 20 Å² to about 250 Å² (e.g. from about 20 Å² to about 40 Å², to about 60 Å², to about 80 Å², to about 100 Å², to about 120 Å², to about 140 Å², to about 160 Å², to about 180 Å², or to about 200 Å²). In some embodiments, C5-interacting compounds may have TPSA of from about 40 Å² to about 60 Å², to about 80 Å², to about 100 Å², to about 120 Å², to about 140 Å², to about 160 Å², or to about 180 Å². In certain embodiments, the compounds may have a TPSA of from about 40 A2 to about 180 Å². As used herein, the term TPSA refers to a predicted sum of surfaces of polar atoms in a molecule.

C5-interacting compounds may bind C5. C5 binding may be characterized, for example, by the equilibrium dissociation constant (K_{D}) for interactions between compounds and C5. In some embodiments, K_{D} values may be obtained by surface plasmon resonance (SPR) analysis. C5-interacting compounds of the present disclosure may exhibit a K_{D} for interactions with C5 of from about 0.01 nM to about 10 nM, from about 0.1 nM to about 20 nM, from about 0.5 nM to about 50 nM, from about 1 nM to about 100 nM, from about 50 nM to about 500 nM, from about 200 nM to about 2000 nM, from about 500 nM to about 5000 nM or from about 1000 nM to about 10000 nM.

In some embodiments, C5 binding may be evaluated using fluorescence polarization. According to such methods, displacement of a fluorescent C5-binding probe may be assessed.

In some embodiments, small molecule C5-interacting compounds may have properties that offer benefits over biomolecule inhibitors. These may include, but are not limited to, increased membrane permeability and solubility. Membrane permeable small molecules may diffuse in a short period of time inside the cell and therefore provide faster therapeutic effect. Small molecules are, in general, more cost effective due to lower production cost and easier storage/shipping (small molecules do not have similar storage/shipping restrictions as biological molecules). Small molecules may be designed to be metabolically stable, have favorable bioavailability, and may be suitable for oral delivery. Additionally, small molecules may be designed to be more suitable for different delivery paths, e.g. topical, ocular, intravenous, or subcutaneous.

### C5 Inhibitors

In some embodiments, C5-interacting compounds of the present disclosure include C5 inhibitors.

Some urea-derivative

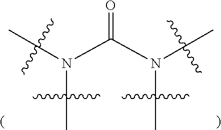 [SMILES: *N(*)C(=O)N(*)*]

complement inhibitors have been described previously (e.g., Zhang et al. in ACS Med. Chem. Lett., 2012, 3(4): 317-21 and International Publication No. WO2013091285, the contents of each of which are herein incorporated by reference in their entirety). These include 1-phenyl-3-(1-phenylethyl) urea derivatives capable of inhibiting C9 deposition through the classical, lectin, and alternative pathways. Such compounds demonstrate selectivity for C9, with no influence on activity of C3 and C4 depositions.

In some embodiments, C5 inhibitor compounds of the disclosure may include any of the compounds presented in International Publication No. WO2013091285, the contents of which are herein incorporated by reference in their entirety. Such compounds include SM0009 of Table 1.

The ability of C5-interacting compounds to inhibit C5 activity may be characterized by hemolysis assay. Hemolysis assays may include different formats, but generally involve analyzing the effect of one or more factors on red blood cell lysis. According to some assays, red blood cells may be used that have been sensitized to ensure susceptibility to C5 activity. Such cells may include antibody-sensitized sheep erythrocytes. Sensitized red blood cells may be combined with C5 protein and other complement components in the presence or absence of C5 inhibitors and the level of red blood cell hemolysis may be measured (e.g., through spectrophotometric analysis). Results may be used to characterize inhibitors by half maximal inhibitory concentration (IC₅₀), where the IC₅₀ represents the concentration of inhibitor needed to reduce hemolysis by half. In some embodiments, C5 inhibitor compounds of the present disclosure may inhibit red blood cell lysis with an IC₅₀ of from about 0.01 nM to about 1 nM, from about 0.1 nM to about 10 nM, from about 0.5 nM to about 50 nM, from about 1 nM to about 100 nM, from about 5 nM to about 500 nM, from about 50 nM to about 1000 nM, from about 200 nM to about 2000 nM, from about 400 nM to about 4000 nM, from about 800 nM to about 8000 nM, from about 2500 nM to about 10000 nM, or from about 5000 nM to about 20000 nM.

In some embodiments, inhibition by C5-interacting compounds may be evaluated by analyzing one or more products of C5 activity. Analysis of C5 activity products may be carried out using standard immunological assays known in the art. Such products may include C5 cleavage products (e.g., C5a or C5b). In some cases, membrane attack complex (MAC) formation is assessed.

In some embodiments, C5 inhibitors may be evaluated for inhibition of C5 activity arising from specific pathways of activation. Such pathways may include classical, alternative, and lectin pathways. Inhibition of specific pathways may be analyzed according to standard procedures known in the art.

In some embodiments, C5 inhibitor compounds of the present disclosure may be optimized to improve solubility. In some cases, C5 inhibitor compounds with enhanced solubility may demonstrate improved inhibition of C5 activity.

In some embodiments, C5 inhibitor compounds of the present disclosure may be optimized to modulate bioavailability. As used herein, the term “bioavailability” refers to the fraction of an administered compound that reaches the systemic circulation. The bioavailability of an intravenously administered compound is near 100%, whereas the percentage may be lower, for example, with orally or topically administered compounds due to incomplete absorption. Bioavailability may be determined, for example, by conducting Drug Metabolism and Pharmacokinetics (DMPK) studies. Such studies may be carried out in vivo. In some embodiments, the bioavailability of C5 inhibitor compounds ranges from about 10% to about 100%, e.g. about 10%, 20%, 30%, 40%, 50%, 60%, 70%, 80%, 90%, or about 100%. In some embodiments, the bioavailability is about 30%.

In some embodiments, C5 inhibitor compounds of the present disclosure may have a K_{D} for C5 of from about 0.01 nM to about 10 nM, from about 0.1 nM to about 20 nM, from about 0.5 nM to about 50 nM, from about 1 nM to about 100 nM, from about 50 nM to about 500 nM, from about 200 nM to about 2000 nM, from about 500 nM to about 5000 nM or from about 1000 nM to about 10000 nM.

In some embodiments, C5 inhibitor compounds of the present disclosure may bind to C5 with a half-maximal effective concentration (EC₅₀) of from about 0.01 nM to about 10 nM, from about 0.1 nM to about 20 nM, from about 10 nM to about 50 nM, from about 20 nM to about 40 nM, from about 30 nM to about 60 nM, from about 50 nM to about 80 nM, from about 75 nM to about 100 nM, from about 90 nM to about 120 nM, from about 110 nM to about 140 nM, from about 130 nM to about 160 nM, from about 150 nM to about 180 nM, from about 170 nM to about 200 nM, from about 190 nM to about 220 nM, from about 210 nM to about 240 nM, from about 230 nM to about 260 nM, from about 250 nM to about 280 nM, from about 270 nM to about 300 nM, from about 290 nM to about 320 nM, from about 310 nM to about 340 nM, from about 330 nM to about 360 nM, from about 350 nM to about 380 nM, from about 370 nM to about 400 nM, from about 390 nM to about 420 nM, from about 410 nM to about 440 nM, from about 430 nM to about 460 nM, from about 450 nM to about 480 nM, from about 470 nM to about 500 nM, from about 200 nM to about 2000 nM, from about 500 nM to about 5000 nM, or from about 1000 nM to about 10000 nM.

In some embodiments, C5 inhibitor compounds of the present disclosure may inhibit red blood cell lysis with an IC₅₀ of from about 0.01 nM to about 10 nM, from about 0.1 nM to about 20 nM, from about 10 nM to about 50 nM, from about 20 nM to about 40 nM, from about 30 nM to about 60 nM, from about 50 nM to about 80 nM, from about 75 nM to about 100 nM, from about 90 nM to about 120 nM, from about 110 nM to about 140 nM, from about 130 nM to about 160 nM, from about 150 nM to about 180 nM, from about 170 nM to about 200 nM, from about 190 nM to about 220 nM, from about 210 nM to about 240 nM, from about 230 nM to about 260 nM, from about 250 nM to about 280 nM, from about 270 nM to about 300 nM, from about 290 nM to about 320 nM, from about 310 nM to about 340 nM, from about 330 nM to about 360 nM, from about 350 nM to about 380 nM, from about 370 nM to about 400 nM, from about 390 nM to about 420 nM, from about 410 nM to about 440 nM, from about 430 nM to about 460 nM, from about 450 nM to about 480 nM, from about 470 nM to about 500 nM, from about 200 nM to about 2000 nM, from about 500 nM to about 5000 nM, or from about 1000 nM to about 10000 nM.

In some embodiments, C5 inhibitor compounds of the present disclosure may inhibit the production of C5a with an IC₅₀ of from about 0.01 nM to about 10 nM, from about 0.1 nM to about 20 nM, from about 10 nM to about 50 nM, from about 20 nM to about 40 nM, from about 30 nM to about 60 nM, from about 50 nM to about 80 nM, from about 75 nM to about 100 nM, from about 90 nM to about 120 nM, from about 110 nM to about 140 nM, from about 130 nM to about 160 nM, from about 150 nM to about 180 nM, from about 170 nM to about 200 nM, from about 190 nM to about 220 nM, from about 210 nM to about 240 nM, from about 230 nM to about 260 nM, from about 250 nM to about 280 nM, from about 270 nM to about 300 nM, from about 290 nM to about 320 nM, from about 310 nM to about 340 nM, from about 330 nM to about 360 nM, from about 350 nM to about 380 nM, from about 370 nM to about 400 nM, from about 390 nM to about 420 nM, from about 410 nM to about 440 nM, from about 430 nM to about 460 nM, from about 450 nM to about 480 nM, from about 470 nM to about 500 nM, from about 200 nM to about 2000 nM, from about 500 nM to about 5000 nM, or from about 1000 nM to about 10000 nM.

In some embodiments, C5 inhibitor compounds of the present disclosure may inhibit membrane attack complex (MAC) formation with an IC₅₀ of from about 0.01 nM to about 10 nM, from about 0.1 nM to about 20 nM, from about 10 nM to about 50 nM, from about 20 nM to about 40 nM, from about 30 nM to about 60 nM, from about 50 nM to about 80 nM, from about 75 nM to about 100 nM, from about 90 nM to about 120 nM, from about 110 nM to about 140 nM, from about 130 nM to about 160 nM, from about 150 nM to about 180 nM, from about 170 nM to about 200 nM, from about 190 nM to about 220 nM, from about 210 nM to about 240 nM, from about 230 nM to about 260 nM, from about 250 nM to about 280 nM, from about 270 nM to about 300 nM, from about 290 nM to about 320 nM, from about 310 nM to about 340 nM, from about 330 nM to about 360 nM, from about 350 nM to about 380 nM, from about 370 nM to about 400 nM, from about 390 nM to about 420 nM, from about 410 nM to about 440 nM, from about 430 nM to about 460 nM, from about 450 nM to about 480 nM, from about 470 nM to about 500 nM, from about 200 nM to about 2000 nM, from about 500 nM to about 5000 nM, or from about 1000 nM to about 10000 nM.

In some embodiments, C5 inhibitor compounds of the present disclosure may inhibit the mannose-binding lectin (MBL) complement pathway with an IC₅₀ of from about 0.01 nM to about 10 nM, from about 0.1 nM to about 20 nM, from about 10 nM to about 50 nM, from about 20 nM to about 40 nM, from about 30 nM to about 60 nM, from about 50 nM to about 80 nM, from about 75 nM to about 100 nM, from about 90 nM to about 120 nM, from about 110 nM to about 140 nM, from about 130 nM to about 160 nM, from about 150 nM to about 180 nM, from about 170 nM to about 200 nM, from about 190 nM to about 220 nM, from about 210 nM to about 240 nM, from about 230 nM to about 260 nM, from about 250 nM to about 280 nM, from about 270 nM to about 300 nM, from about 290 nM to about 320 nM, from about 310 nM to about 340 nM, from about 330 nM to about 360 nM, from about 350 nM to about 380 nM, from about 370 nM to about 400 nM, from about 390 nM to about 420 nM, from about 410 nM to about 440 nM, from about 430 nM to about 460 nM, from about 450 nM to about 480 nM, from about 470 nM to about 500 nM, from about 200 nM to about 2000 nM, from about 500 nM to about 5000 nM, or from about 1000 nM to about 10000 nM.

In some embodiments, the C5 inhibitor compounds may inhibit the alternative complement pathway with an IC₅₀ of from about 0.01 nM to about 10 nM, from about 0.1 nM to about 20 nM, from about 10 nM to about 50 nM, from about 20 nM to about 40 nM, from about 30 nM to about 60 nM, from about 50 nM to about 80 nM, from about 75 nM to about 100 nM, from about 90 nM to about 120 nM, from about 110 nM to about 140 nM, from about 130 nM to about 160 nM, from about 150 nM to about 180 nM, from about 170 nM to about 200 nM, from about 190 nM to about 220 nM, from about 210 nM to about 240 nM, from about 230 nM to about 260 nM, from about 250 nM to about 280 nM, from about 270 nM to about 300 nM, from about 290 nM to about 320 nM, from about 310 nM to about 340 nM, from about 330 nM to about 360 nM, from about 350 nM to about 380 nM, from about 370 nM to about 400 nM, from about 390 nM to about 420 nM, from about 410 nM to about 440 nM, from about 430 nM to about 460 nM, from about 450 nM to about 480 nM, from about 470 nM to about 500 nM, from about 200 nM to about 2000 nM, from about 500 nM to about 5000 nM, or from about 1000 nM to about 10000 nM.

### Generic Structures

In some embodiments, the structures of the C5-interacting compounds (such as C5 inhibitors) of the present disclosure may be encompassed by the generic structure of Formula (100):

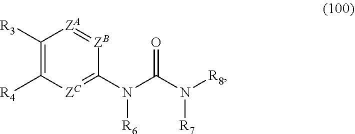

or a pharmaceutically acceptable salt thereof, wherein
Z^{A} is N or CR2, Z^{B} is N or CR1, Z^{C} is N or CR5, with a proviso that
when Z^{A} is N, then Z^{B}═CR₁ and Z^{C}═CR₅;
when Z^{B} is N, then Z^{A}═CR₂ and Z^{C}═CR₅;
when Z^{C} is N, then Z^{A}═CR₂ and Z^{B}═CR₁; and
when both Z^{B} and Z^{C} are N, then Z^{A}═CR₂;
and wherein
R1, R2, or R5 is independently H, alkyl, cyclic alkyl, alkenyl, alkynyl, halogen, hydroxyl, alkoxyl, ether, CN, amine, amide, aryl, or heteroaryl, wherein the alkyl, cyclic alkyl, alkenyl, alkynyl, alkoxyl, ether, amine, aryl, or heteroaryl group is optionally substituted; R3 or R4 is independently alkyl, cyclic alkyl, alkenyl, alkynyl, halogen, hydroxyl, alkoxyl, ether, CN, amine, amide, aryl, or heteroaryl, wherein the alkyl, cyclic alkyl, alkenyl, alkynyl, alkoxyl, ether, amine, aryl, or heteroaryl group is optionally substituted; R6 or R7 is independently H or an alkyl group, wherein the alkyl group is optional substituted; optionally, R6 and R7, together with the nitrogens to which they are attached and the carbonyl group, may form a 5 to 7-membered heterocycle which may be optionally substituted;
R8 is an alkyl group, wherein the alkyl group is optional substituted; optionally, R8 and R7, together with the nitrogen to which they are attached, may for a 5 to 6-membered heterocycle which may be optionally substituted.

In some embodiments, R1, R2 and R5 are H.

In some embodiments, R3 is —OCH3.

In some embodiments, R4 is an alkoxyl group such or

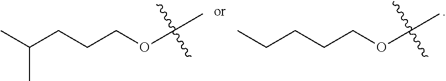

In some embodiments, R8 is a substituted alkyl group with a structure of

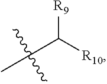

wherein R9 or R10 independently is H, alkyl, alkenyl, alkynyl, halogen, hydroxyl, alkoxyl, ether, CN, amine, aryl, heteroaryl, cyclic alkyl, heterocyclic alkyl, multicyclic alkyl group, or hetero multicyclic alkyl group, wherein the alkyl, alkenyl, alkynyl, alkoxy, ether, amine, aryl, heteroaryl group, cyclic alkyl, heterocyclic alkyl, multicyclic alkyl, or hetero multicyclic alkyl group is optional substituted. In some embodiments, R9 and R10, together with the carbon they are attached, form a 3 to 8 membered cyclic or heterocyclic group, wherein the cyclic or heterocyclic group may be optionally substituted.

In some embodiments, R9 is an alkyl group.

In some embodiments, R9 is H.

In some embodiments, R10 is an optionally substituted cyclic group. The cyclic group may be saturated, aromatic, non-aromatic, unsaturated, or partially unsaturated. The cyclic group may be aryl, heteroaryl, multicyclic, or multi-heterocyclic. The heteroatom of the heteroaryl or multi-heterocyclic group may be O, N, or S.

In some embodiments, R8 is not

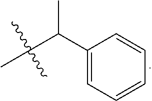 [SMILES: *C(C)c1ccccc1]

In some embodiments, the structures of the C5-interacting compounds (such as C5 inhibitors) of the present disclosure may be encompassed by the generic structure of Formula (200):

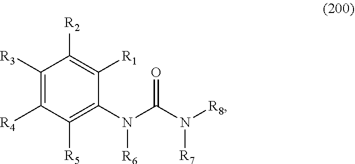

or a pharmaceutically acceptable salt thereof, wherein R1, R2, or R5 is independently H, alkyl, cyclic alkyl, alkenyl, alkynyl, halogen, hydroxyl, alkoxyl, ether, CN, amine, amide, aryl, or heteroaryl, wherein the alkyl, cyclic alkyl, alkenyl, alkynyl, alkoxyl, ether, amine, aryl, or heteroaryl group is optionally substituted; R3 or R4 is independently alkyl, cyclic alkyl, alkenyl, alkynyl, halogen, hydroxyl, alkoxyl, ether, CN, amine, amide, aryl, or heteroaryl, wherein the alkyl, cyclic alkyl, alkenyl, alkynyl, alkoxyl, ether, amine, aryl, or heteroaryl group is optionally substituted; R6 or R7 is independently H or an alkyl group, wherein the alkyl group is optional substituted; optionally, R6 and R7, together with the nitrogens to which they are attached and the carbonyl group, may form a 5 to 7-membered heterocycle which may be optionally substituted;
R8 is an alkyl group, wherein the alkyl group is optional substituted; optionally, R8 and R7, together with the nitrogen to which they are attached, may for a 5 to 6-membered heterocycle which may be optionally substituted.

In some embodiments, R1, R2 and R5 are H.

In some embodiments, R3 is —OCH3.

In some embodiments, R4 is an alkoxyl group such as or

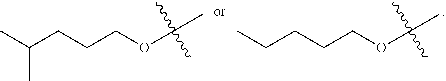

In some embodiments, R8 is a substituted alkyl group with a structure of

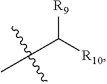

wherein R9 or R10 independently is H, alkyl, alkenyl, alkynyl, halogen, hydroxyl, alkoxyl, ether, CN, amine, aryl, heteroaryl, cyclic alkyl, heterocyclic alkyl, multicyclic alkyl group, or hetero multicyclic alkyl group, wherein the alkyl, alkenyl, alkynyl, alkoxy, ether, amine, aryl, heteroaryl group, cyclic alkyl, heterocyclic alkyl, multicyclic alkyl, or hetero multicyclic alkyl group is optional substituted. In some embodiments, R9 and R10, together with the carbon they are attached, form a 3 to 8 membered cyclic or heterocyclic group, wherein the cyclic or heterocyclic group may be optionally substituted.

In some embodiments, R9 is an alkyl group.

In some embodiments, R9 is H.

In some embodiments, R10 is an optionally substituted cyclic group. The cyclic group may be saturated, aromatic, non-aromatic, unsaturated, or partially unsaturated. The cyclic group may be aryl, heteroaryl, multicyclic, or multi-heterocyclic. The heteroatom of the heteroaryl or multi-heterocyclic group may be O, N, or S.

In some embodiments, R8 is not

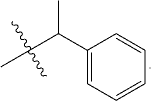 [SMILES: *C(C)c1ccccc1]

In some embodiments, the structures of the C5-interacting compounds (such as C5 inhibitors) of the present disclosure may be encompassed by the generic structure of Formula (300):

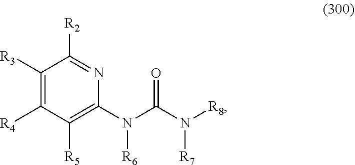

or a pharmaceutically acceptable salt thereof, wherein R2 or R5 is independently H, alkyl, cyclic alkyl, alkenyl, alkynyl, halogen, hydroxyl, alkoxyl, ether, CN, amine, amide, aryl, or heteroaryl, wherein the alkyl, cyclic alkyl, alkenyl, alkynyl, alkoxyl, ether, amine, aryl, or heteroaryl group is optionally substituted;
R3 or R4 is independently alkyl, cyclic alkyl, alkenyl, alkynyl, halogen, hydroxyl, alkoxyl, ether, CN, amine, amide, aryl, or heteroaryl, wherein the alkyl, cyclic alkyl, alkenyl, alkynyl, alkoxyl, ether, amine, aryl, or heteroaryl group is optionally substituted;
R6 or R7 is independently H or an alkyl group, wherein the alkyl group is optional substituted; optionally, R6 and R7, together with the nitrogens to which they are attached and the carbonyl group, may form a 5 to 7-membered heterocycle which may be optionally substituted;
R8 is an alkyl group, wherein the alkyl group is optional substituted; optionally, R8 and R7, together with the nitrogen to which they are attached, may for a 5 to 6-membered heterocycle which may be optionally substituted.

In some embodiments, R2 and R5 are H.

In some embodiments, R3 is —OCH3.

In some embodiments, R4 is an alkoxyl group such as or

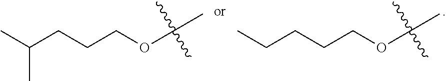

In some embodiments, R8 is a substituted alkyl group with a structure of

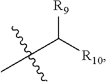

wherein R9 or R10 independently is H, alkyl, alkenyl, alkynyl, halogen, hydroxyl, alkoxyl, ether, CN, amine, aryl, heteroaryl, cyclic alkyl, heterocyclic alkyl, multicyclic alkyl group, or hetero multicyclic alkyl group, wherein the alkyl, alkenyl, alkynyl, alkoxy, ether, amine, aryl, heteroaryl group, cyclic alkyl, heterocyclic alkyl, multicyclic alkyl, or hetero multicyclic alkyl group is optional substituted. In some embodiments, R9 and R10, together with the carbon they are attached, form a 3 to 8 membered cyclic or heterocyclic group, wherein the cyclic or heterocyclic group may be optionally substituted.

In some embodiments, R9 is an alkyl group.

In some embodiments, R9 is H.

In some embodiments, R10 is an optionally substituted cyclic group. The cyclic group may be saturated, aromatic, non-aromatic, unsaturated, or partially unsaturated. The cyclic group may be aryl, heteroaryl, multicyclic, or multi-heterocyclic. The heteroatom of the heteroaryl or multi-heterocyclic group may be O, N, or S.

In some embodiments, R8 is not

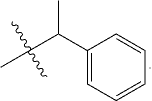 [SMILES: *C(C)c1ccccc1]

In some embodiments, the structures of the C5-interacting compounds (such as C5 inhibitors) of the present disclosure may be encompassed by the generic structure of Formula

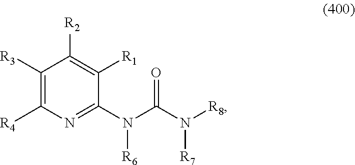

or a pharmaceutically acceptable salt thereof, wherein R1 or R2 is independently H, alkyl, cyclic alkyl, alkenyl, alkynyl, halogen, hydroxyl, alkoxyl, ether, CN, amine, amide, aryl, or heteroaryl, wherein the alkyl, cyclic alkyl, alkenyl, alkynyl, alkoxyl, ether, amine, aryl, or heteroaryl group is optionally substituted;
R3 or R4 is independently alkyl, cyclic alkyl, alkenyl, alkynyl, halogen, hydroxyl, alkoxyl, ether, CN, amine, amide, aryl, or heteroaryl, wherein the alkyl, cyclic alkyl, alkenyl, alkynyl, alkoxyl, ether, amine, aryl, or heteroaryl group is optionally substituted;
R6 or R7 is independently H or an alkyl group, wherein the alkyl group is optional substituted; optionally, R6 and R7, together with the nitrogens to which they are attached and the carbonyl group, may form a 5 to 7-membered heterocycle which may be optionally substituted;
R8 is an alkyl group, wherein the alkyl group is optional substituted; optionally, R8 and R7, together with the nitrogen to which they are attached, may for a 5 to 6-membered heterocycle which may be optionally substituted.

In some embodiments, R1 and R2 are H.

In some embodiments, R3 is —OCH3.

In some embodiments, R4 is an alkoxyl group such as or

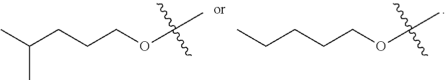

In some embodiments, R8 is a substituted alkyl group with a structure of

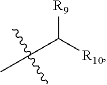

wherein R9 or R10 independently is H, alkyl, alkenyl, alkynyl, halogen, hydroxyl, alkoxyl, ether, CN, amine, aryl, heteroaryl, cyclic alkyl, heterocyclic alkyl, multicyclic alkyl group, or hetero multicyclic alkyl group, wherein the alkyl, alkenyl, alkynyl, alkoxy, ether, amine, aryl, heteroaryl group, cyclic alkyl, heterocyclic alkyl, multicyclic alkyl, or hetero multicyclic alkyl group is optional substituted. In some embodiments, R9 and R10, together with the carbon they are attached, form a 3 to 8 membered cyclic or heterocyclic group, wherein the cyclic or heterocyclic group may be optionally substituted.

In some embodiments, R9 is an alkyl group.

In some embodiments, R9 is H.

In some embodiments, R10 is an optionally substituted cyclic group. The cyclic group may be saturated, aromatic, non-aromatic, unsaturated, or partially unsaturated. The cyclic group may be aryl, heteroaryl, multicyclic, or multi-heterocyclic. The heteroatom of the heteroaryl or multi-heterocyclic group may be O, N, or S.

In some embodiments, R8 is not

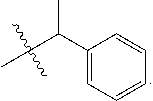 [SMILES: *C(C)c1ccccc1]

In some embodiments, the structures of the C5-interacting compounds (such as C5 inhibitors of the resent disclosure may be encompassed by the generic structure of Formula

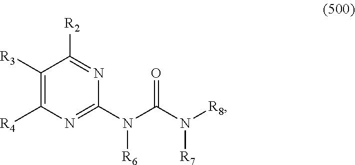

or a pharmaceutically acceptable salt thereof, wherein R2 is H, alkyl, cyclic alkyl, alkenyl, alkynyl, halogen, hydroxyl, alkoxyl, ether, CN, amine, amide, aryl, or heteroaryl, wherein the alkyl, cyclic alkyl, alkenyl, alkynyl, alkoxyl, ether, amine, aryl, or heteroaryl group is optionally substituted;
R3 or R4 is independently alkyl, cyclic alkyl, alkenyl, alkynyl, halogen, hydroxyl, alkoxyl, ether, CN, amine, amide, aryl, or heteroaryl, wherein the alkyl, cyclic alkyl, alkenyl, alkynyl, alkoxyl, ether, amine, aryl, or heteroaryl group is optionally substituted; R6 or R7 is independently H or an alkyl group, wherein the alkyl group is optional substituted, and wherein R6 and R7, together with the nitrogens to which they are attached and the carbonyl group, may form a 5 to 7-membered heterocycle which may be optionally substituted;
R8 is an alkyl group, wherein the alkyl group is optional substituted; optionally, R8 and R7, together with the nitrogen to which they are attached, may for a 5 to 6-membered heterocycle which may be optionally substituted.

In some embodiments, R2 is H.

In some embodiments, R3 is —OCH3.

In some embodiments, R4 is an alkoxyl group such as or

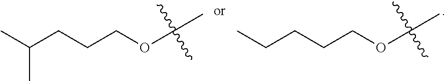

In some embodiments, R8 is a substituted alkyl group with a structure of

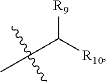

wherein R9 or R10 independently is H, alkyl, alkenyl, alkynyl, halogen, hydroxyl, alkoxyl, ether, CN, amine, aryl, heteroaryl, cyclic alkyl, heterocyclic alkyl, multicyclic alkyl group, or hetero multicyclic alkyl group, wherein the alkyl, alkenyl, alkynyl, alkoxy, ether, amine, aryl, heteroaryl group, cyclic alkyl, heterocyclic alkyl, multicyclic alkyl, or hetero multicyclic alkyl group is optional substituted. In some embodiments, R9 and R10, together with the carbon they are attached, form a 3 to 8 membered cyclic or heterocyclic group, wherein the cyclic or heterocyclic group may be optionally substituted.

In some embodiments, R9 is an alkyl group.

In some embodiments, R9 is H.

In some embodiments, R10 is an optionally substituted cyclic group. The cyclic group may be saturated, aromatic, non-aromatic, unsaturated, or partially unsaturated. The cyclic group may be aryl, heteroaryl, multicyclic, or multi-heterocyclic. The heteroatom of the heteroaryl or multi-heterocyclic group may be O, N, or S.

In some embodiments, R8 is not

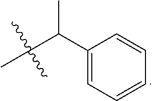 [SMILES: *C(C)c1ccccc1]

In some embodiments, the structures of the C5-interacting compounds (such as C5 inhibitors of the present disclosure may be encompassed by the generic structure of Formula (600):

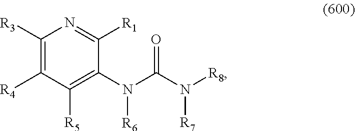

or a pharmaceutically acceptable salt thereof, wherein R1 or R5 is independently H, alkyl, cyclic alkyl, alkenyl, alkynyl, halogen, hydroxyl, alkoxyl, ether, CN, amine, amide, aryl, or heteroaryl, wherein the alkyl, cyclic alkyl, alkenyl, alkynyl, alkoxyl, ether, amine, aryl, or heteroaryl group is optionally substituted;
R3 or R4 is independently alkyl, cyclic alkyl, alkenyl, alkynyl, halogen, hydroxyl, alkoxyl, ether, CN, amine, amide, aryl, or heteroaryl, wherein the alkyl, cyclic alkyl, alkenyl, alkynyl, alkoxyl, ether, amine, aryl, or heteroaryl group is optionally substituted; R6 or R7 is independently H or an alkyl group, wherein the alkyl group is optional substituted, and wherein R6 and R7, together with the nitrogens to which they are attached and the carbonyl group, may form a 5 to 7-membered heterocycle which may be optionally substituted;
R8 is an alkyl group, wherein the alkyl group is optional substituted; optionally, R8 and R7, together with the nitrogen to which they are attached, may for a 5 to 6-membered heterocycle which may be optionally substituted.

In some embodiments, R2 is H.

In some embodiments, R3 is —OCH3.

In some embodiments, R4 is an alkoxyl group such as or

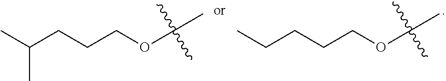

In some embodiments, R8 is a substituted alkyl group with a structure of

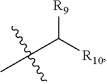

wherein R9 or R10 independently is H, alkyl, alkenyl, alkynyl, halogen, hydroxyl, alkoxyl, ether, CN, amine, aryl, heteroaryl, cyclic alkyl, heterocyclic alkyl, multicyclic alkyl group, or hetero multicyclic alkyl group, wherein the alkyl, alkenyl, alkynyl, alkoxy, ether, amine, aryl, heteroaryl group, cyclic alkyl, heterocyclic alkyl, multicyclic alkyl, or hetero multicyclic alkyl group is optional substituted. In some embodiments, R9 and R10, together with the carbon they are attached, form a 3 to 8 membered cyclic or heterocyclic group, wherein the cyclic or heterocyclic group may be optionally substituted.

In some embodiments, R9 is an alkyl group.

In some embodiments, R9 is H.

In some embodiments, R10 is an optionally substituted cyclic group. The cyclic group may be saturated, aromatic, non-aromatic, unsaturated, or partially unsaturated. The cyclic group may be aryl, heteroaryl, multicyclic, or multi-heterocyclic. The heteroatom of the heteroaryl or multi-heterocyclic group may be O, N, or S.

In some embodiments, R8 is not

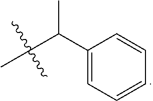 [SMILES: *C(C)c1ccccc1]

In some embodiments, the structures of the C5-interacting compounds (such as C5 inhibitors of the resent disclosure may be encompassed b the generic structure of Formula (700):

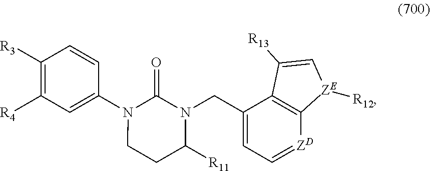

or a pharmaceutically acceptable salt thereof, wherein
R3 or R4 is independently alkyl, cyclic alkyl, alkenyl, alkynyl, halogen, hydroxyl, alkoxyl, ether, CN, amine, amide, aryl, or heteroaryl, wherein the alkyl, cyclic alkyl, alkenyl, alkynyl, alkoxyl, ether, amine, aryl, or heteroaryl group is optionally substituted;
R11 is H or an alkyl group, wherein the alkyl group is optional substituted;
R12 is H, alkyl, alkenyl, alkynyl, halogen, hydroxyl, alkoxyl, ether, CN, amine, amide, aryl, heteroaryl, cyclic alkyl, heterocyclic alkyl, multicyclic alkyl group, or hetero multicyclic alkyl group, wherein the alkyl, alkenyl, alkynyl, alkoxy, ether, amine, aryl, heteroaryl group, cyclic alkyl, heterocyclic alkyl, multicyclic alkyl, or hetero multicyclic alkyl group is optional substituted;
R13 is H, halogen, —CN, —CF3, or a C1-C3 alkyl group;
Z^{D} is selected from N or CR₁₄, wherein R14 is H or an alkyl group, wherein the alkyl group is optionally substituted; and
Z^{E} is selected from N or CH.

In some embodiments, R3 and R4 are independently C3-C8 alkyl, C3-C8 cyclic alkyl, C3-C8 alkenyl, C3-C8 alkynyl, halogen, hydroxyl, C3-C8 alkoxyl, aryl, or heteroaryl group.

In some embodiments, R3 is —OCH3.

In some embodiments, R4 is an alkoxyl group such as or

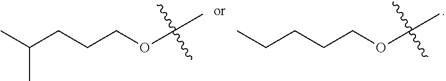

In some embodiments, R12 comprises an amide group.

Non-limiting examples of C5 inhibitor compounds having a structure according to Formula (700) include CU0019-CU0032, CU0035, CU0036, CU0039, CU0041, CU0043, CU0044, CU0046, CU0048-CU0051, CU0053, CU0054, CU0056, CU0057, CU0059-CU0062, CU00228, CU0229, CU0231, CU0232, CU0234, CU0235, CU0239-CU0262, CU00500, CU0502, CU0504, CU0506, CU0508-CU0510, CU0513-CU0516, CU0518-CU0530, CU0532-CU0535, CU0538-CU0541, CU0543, CU-0547, CU0549, CU0551, CU0553, CU0556, CU0557, CU0559-CU0561, CU0563, CU0564, CU0566, CU0567, CU0569, CU0570, CU0572, CU0575-CU0577, CU0580-CU0583, CU0588, CU0590, CU0591, CU0593, CU0595, CU0599, CU0600, CU0602-CU0604, CU0606, CU0610, CU0612, CU0620, CU0622, CU0623, CU0625, CU0627, CU0629, CU0630, CU0631, CU0633, CU0637, CU0639-CU0641, CU0644, CU0646, CU0653, CU0654, CU0656, CU0658, CU0665-CU0667, CU0675, CU0678, CU0681, CU0683, CU0684, CU0689, CU0692, CU0694, CU0696, CU0703, CU0705, CU0707, CU0710, CU0714, CU0730, CU0735, CU0737, CU0745, CU0747, CU0749, CU0751-CU0753, CU0756, CU0761, CU0764-CU0767, CU0773, CU0777, CU0778, CU0780, CU0781, CU0785, CU0786, CU0789, CU0790, CU0792-CU0795, CU0798-CU0801, CU0803, CU0804, CU0806, CU0811, CU0812, CU0817, CU0818, CU0820-CU0822, CU0824, CU0828, CU0829, CU0831, CU0835, CU0837, CU0842, CU0843, CU0845-CU0847, SC0001-SC0015, SC0100, SC0103, SC0105-SC0113, SC0115-SC0117, SC0119, SC0120, SC0122, SC0124, SC0125, SC0127-SC0129, SC0131, SC0133, SC0143, SC0145, SC0147, SC0150, SC0151, SC0154-SC0156, SC0164, SC0167-SC0169, SC0171, SC0174, SC0177, SC0203, SC0205, SC0208, SC0211, SC0214, SC0219 and SC0227.

In some embodiments, the structures of the C5-interacting compounds (such as C5 inhibitors) of the present disclosure may be encompassed by the generic structure of Formula (701):

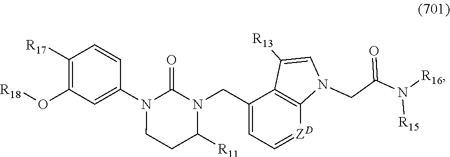

or a pharmaceutically acceptable salt thereof, wherein
R11 is H or a methyl group;
R13 is H, halogen, —CN, —CF3, or a C1-C3 alkyl group;
R15 or R16, independently, is H, alkyl, aryl, heteroaryl, cyclic alkyl, heterocyclic alkyl, multicyclic alkyl group, or hetero multicyclic alkyl group, wherein the alkyl, aryl, heteroaryl group, cyclic alkyl, heterocyclic alkyl, multicyclic alkyl, or hetero multicyclic alkyl group is optional substituted; optionally, R15 and R16, together with the nitrogen they are attached, form a 3 to 8 membered heterocyclic group, wherein the heterocyclic group may be optionally substituted;
R17 is halogen, an alkyl group, or an alkoxyl group;
R18 is an alkyl group; and
Z^{D} is selected from N or CR₁₄, wherein R14 is H or an alkyl group, wherein the alkyl group is optionally substituted.

In some embodiments, R13 is H or Cl.

In some embodiments, R17 is a C3-C8 alkyl, or C3-C8 alkoxyl group.

In some embodiments, R18 is a C3-C8 alkyl group.

In some embodiments, R17 is —OCH3.

In some embodiments, R18 is

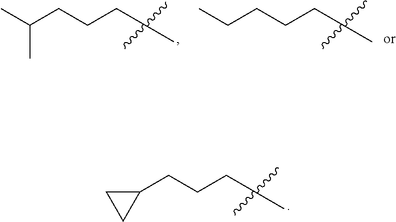

In some embodiments, Z^{D} is selected form N or CH.

In some embodiments, R15 and R16 are both C1-C3 alkyl groups, such as methyl groups.

In some embodiments, R15 and R16, together with the nitrogen they are attached to, form a 6-membered non-aromatic heterocyclic group. In some embodiments, the heterocyclic group is

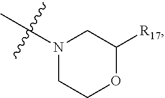

wherein R17 is an alkyl group, wherein the alkyl group is optionally substituted. In some embodiments, R17 is an alkyl group substituted with an amine group.

Non-limiting examples of C5 inhibitor compounds having a structure according to Formula (701) include CU0025-CU0031, CU0035, CU0036, CU0043, CU0044, CU0046, CU0048-CU0051, CU0053, CU0054, CU0056, CU0057, CU0059-CU0062, CU00228, CU0229, CU0231, CU0232, CU0235, CU0239, CU0242-CU0247, CU0252, CU0253, CU0255-CU0262, CU0502, CU0504, CU0506, CU0508-CU0510, CU0515, CU0516, CU0518-CU0524, CU0526, CU0528-CU0530, CU0532-CU0535, CU0538-CU0541, CU0543, CU0549, CU0553, CU0557, CU0560, CU0561, CU0567, CU0572, CU0582, CU0591, CU0595, CU0602, CU0603, CU0606, CU0610, CU0625, CU0656, CU0665, CU0681, CU0694, CU0696, CU0703, CU0707, CU0737, CU0747, CU0752, CU0761, CU0764, CU0765, CU0767, CU0780, CU0790, CU0794, CU0799, CU0800, CU0803, CU0811, CU0817, CU0828, CU0829, CU0843, CU0846, CU0847, SC0001-SC0009, SC0011, SC0012, SC0014, SC0100, SC0103, SC0105-SC0113, SC0115-SC0117, SC0119, SC0120, SC0122, SC0124, SC0125, SC0127-SC0129, SC0133, SC0143, SC0145, SC0147, SC0154-SC0156, SC0164, SC0168, SC0171, SC0174, SC0177, SC0205 and SC0208.

### 1). Linear-Urea C5-Interacting Compounds

In some embodiments, C5-interacting compounds of the present disclosure may include any one of SM0001-SM0121, SM0200-SM0219, C5INH-0294, C5INH-0296, C5INH-0298, C5INH-0303, C5INH-0310, C5INH-0311, C5INH-0315, C5INH-0316, C5INH-0317, C5INH-0318, C5INH-0319, C5INH-0321, C5INH-0323, C5INH-0324, C5INH-0326, C5INH-0329, C5INH-0330, C5INH-0333, C5INH-0335, C5INH-0336, C5INH-0338, C5INH-0339, C5INH-0340, C5INH-0342, C5INH-0343, C5INH-0348, C5INH-0349, C5INH-0350, C5INH-0352, C5INH-0353, C5INH-0355, C5INH-0356, C5INH-0357, C5INH-0361, C5INH-0366, C5INH-0367, C5INH-0369, C5INH-0370, C5INH-0371, C5INH-0372, C5INH-0373, C5INH-0377, C5INH-0379, C5INH-0381, C5INH-0382, C5INH-0383, C5INH-0384, C5INH-0385, C5INH-0387, C5INH-0388, C5INH-0389, C5INH-0390, C5INH-0391, C5INH-0395, C5INH-0396, C5INH-0397, C5INH-0398, C5INH-0399, C5INH-0401, C5INH-0402, C5INH-0403, C5INH-0406, C5INH-0409, C5INH-0410, C5INH-0411, C5INH-0414, C5INH-0417, C5INH-0420, C5INH-0421, C5INH-0422, C5INH-0425, C5INH-0428, C5INH-0431, C5INH-0432, C5INH-0436, C5INH-0437, C5INH-0438, C5INH-0440, C5INH-0443, C5INH-0446, C5INH-0447, C5INH-0448, C5INH-0450, C5INH-0452, C5INH-0453, C5INH-0454, C5INH-0456, C5INH-0458, C5INH-0460, C5INH-0462, C5INH-0463, C5INH-0469, C5INH-0472, C5INH-0473, C5INH-0474, C5INH-0476, C5INH-0477, C5INH-0484, C5INH-0485, C5INH-0486, C5INH-0487, C5INH-0488, C5INH-0489, C5INH-0490, C5INH-0491, C5INH-0492, C5INH-0496, C5INH-0497, C5INH-0498, C5INH-0500, C5INH-0501, C5INH-0502, C5INH-0504, C5INH-0507, C5INH-0508, C5INH-0509, C5INH-0510, C5INH-0512, C5INH-0513, C5INH-0515, C5INH-0516, C5INH-0517, C5INH-0518, C5INH-0519, C5INH-0521, C5INH-0524, C5INH-0525, C5INH-0526, C5INH-0527, C5INH-0532, C5INH-0533, C5INH-0534, C5INH-0535, C5INH-0536, C5INH-0537, C5INH-0538, C5INH-0539, C5INH-0540, C5INH-0541, C5INH-0543, C5INH-0544, C5INH-0545, and C5INH-0547, presented in Table 1, or a pharmaceutically acceptable salt thereof.

TABLE 1
Linear-Urea C5-interacting compounds

| Compound | |
|---|---|
| Number | Structure |
| SM0001 | 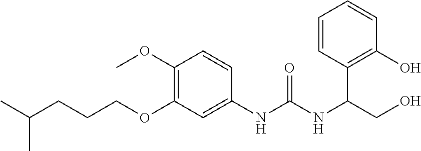 [SMILES: COc1ccc(NC(=O)NC(CO)c2ccccc2O)cc1OCCCC(C)C] |
| SM0002 | 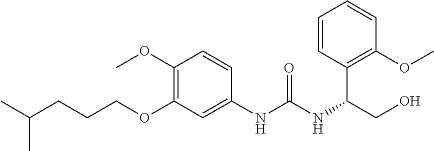 [SMILES: COc1ccc(NC(=O)N[C@@H](CO)c2ccccc2OC)cc1OCCCC(C)C] |
| SM0003 | 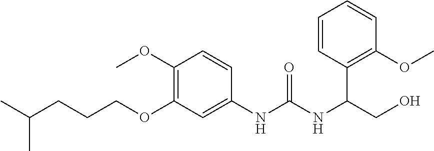 [SMILES: COc1ccc(NC(=O)NC(CO)c2ccccc2OC)cc1OCCCC(C)C] |
| SM0004 | 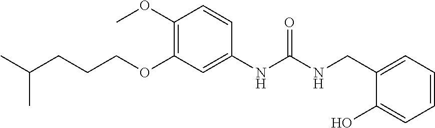 [SMILES: COc1ccc(NC(=O)NCc2ccccc2O)cc1OCCCC(C)C] |
| SM0005 | 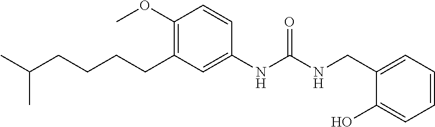 [SMILES: COc1ccc(NC(=O)NCc2ccccc2O)cc1CCCCC(C)C] |
| SM0006 | 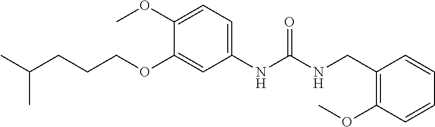 [SMILES: COc1ccccc1CNC(=O)Nc1ccc(OC)c(OCCCC(C)C)c1] |
| SM0007 | 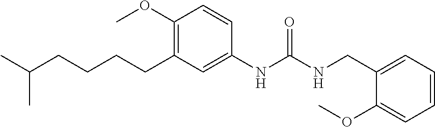 [SMILES: COc1ccc(NC(=O)NCc2ccccc2OC)cc1CCCCC(C)C] |
| SM0008 | 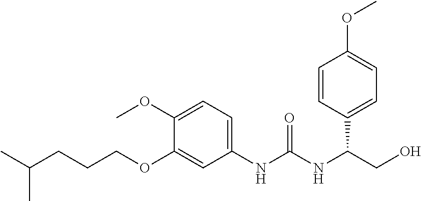 [SMILES: COc1ccc([C@H](CO)NC(=O)Nc2ccc(OC)c(OCCCC(C)C)c2)cc1] |
| SM0009 | 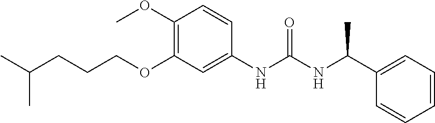 [SMILES: COc1ccc(NC(=O)N[C@@H](C)c2ccccc2)cc1OCCCC(C)C] |
| SM0010 | 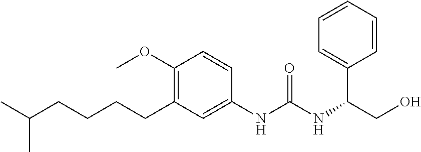 [SMILES: COc1ccc(NC(=O)N[C@@H](CO)c2ccccc2)cc1CCCCC(C)C] |
| SM0011 | 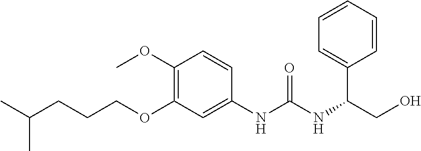 [SMILES: COc1ccc(NC(=O)N[C@@H](CO)c2ccccc2)cc1OCCCC(C)C] |
| SM0012 | 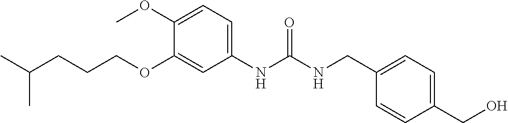 [SMILES: COc1ccc(NC(=O)NCc2ccc(CO)cc2)cc1OCCCC(C)C] |
| SM0013 | 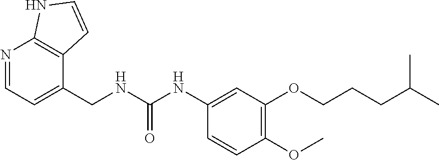 [SMILES: COc1ccc(NC(=O)NCc2ccnc3[nH]ccc23)cc1OCCCC(C)C] |
| SM0014 | 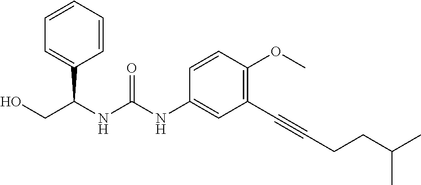 [SMILES: COc1ccc(NC(=O)N[C@@H](CO)c2ccccc2)cc1C#CCCC(C)C] |
| SM0015 | 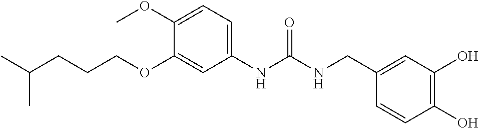 [SMILES: COc1ccc(NC(=O)NCc2ccc(O)c(O)c2)cc1OCCCC(C)C] |
| SM0016 | 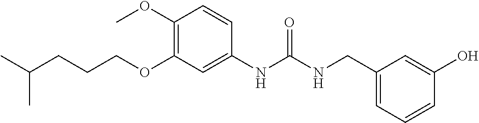 [SMILES: COc1ccc(NC(=O)NCc2cccc(O)c2)cc1OCCCC(C)C] |
| SM0017 | 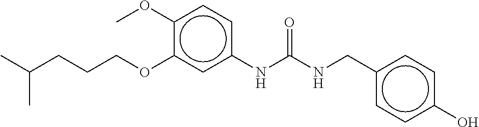 |
| SM0018 | 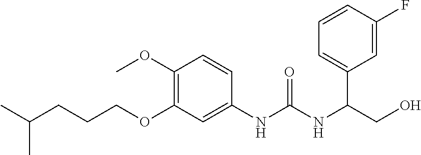 [SMILES: COc1ccc(NC(=O)NC(CO)c2cccc(F)c2)cc1OCCCC(C)C] |
| SM0019 | 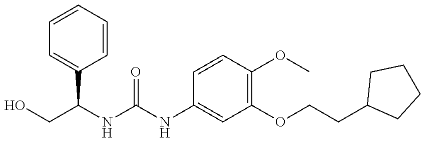 [SMILES: COc1ccc(NC(=O)N[C@@H](CO)c2ccccc2)cc1OCCC1CCCC1] |
| SM0020 | 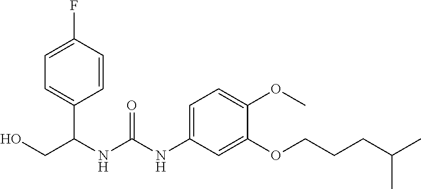 [SMILES: COc1ccc(NC(=O)NC(CO)c2ccc(F)cc2)cc1OCCCC(C)C] |
| SM0021 | 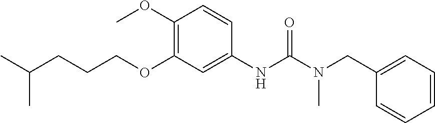 [SMILES: COc1ccc(NC(=O)N(C)Cc2ccccc2)cc1OCCCC(C)C] |
| SM0022 | 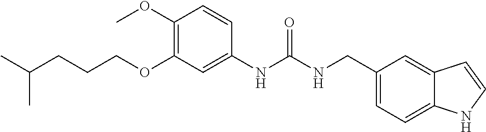 [SMILES: COc1ccc(NC(=O)NCc2ccc3[nH]ccc3c2)cc1OCCCC(C)C] |
| SM0023 | 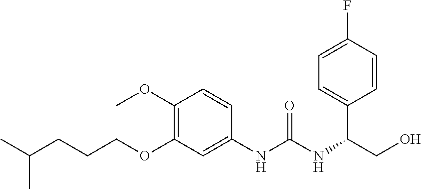 [SMILES: COc1ccc(NC(=O)N[C@@H](CO)c2ccc(F)cc2)cc1OCCCC(C)C] |
| SM0024 | 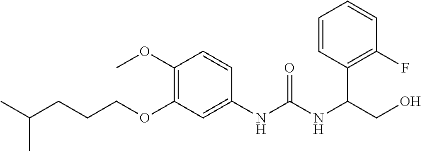 [SMILES: COc1ccc(NC(=O)NC(CO)c2ccccc2F)cc1OCCCC(C)C] |
| SM0025 | 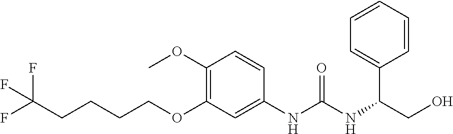 [SMILES: COc1ccc(NC(=O)N[C@@H](CO)c2ccccc2)cc1OCCCCC(F)(F)F] |
| SM0026 | 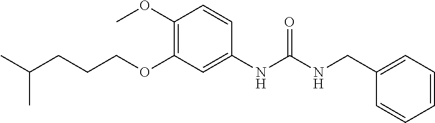 [SMILES: COc1ccc(NC(=O)NCc2ccccc2)cc1OCCCC(C)C] |
| SM0027 | 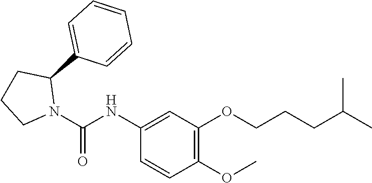 [SMILES: COc1ccc(NC(=O)N2CCC[C@H]2c2ccccc2)cc1OCCCC(C)C] |
| SM0028 | 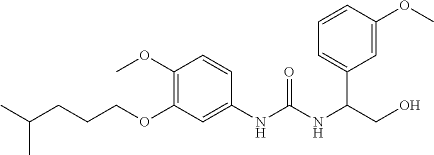 [SMILES: COc1cccc(C(CO)NC(=O)Nc2ccc(OC)c(OCCCC(C)C)c2)c1] |
| SM0029 | 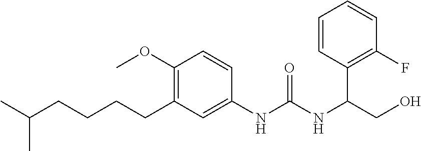 [SMILES: COc1ccc(NC(=O)NC(CO)c2ccccc2F)cc1CCCCC(C)C] |
| SM0030 | 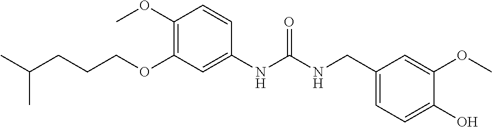 [SMILES: COc1cc(CNC(=O)Nc2ccc(OC)c(OCCCC(C)C)c2)ccc1O] |
| SM0031 | 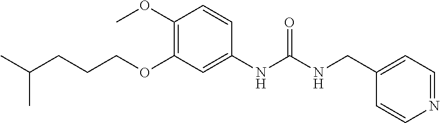 [SMILES: COc1ccc(NC(=O)NCc2ccncc2)cc1OCCCC(C)C] |
| SM0032 | 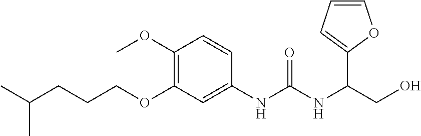 [SMILES: COc1ccc(NC(=O)NC(CO)c2ccco2)cc1OCCCC(C)C] |
| SM0033 | 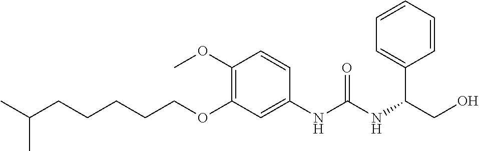 [SMILES: COc1ccc(NC(=O)N[C@@H](CO)c2ccccc2)cc1OCCCCCC(C)C] |
| SM0034 | 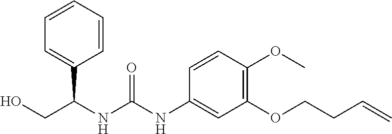 [SMILES: C=CCCOc1cc(NC(=O)N[C@@H](CO)c2ccccc2)ccc1OC] |
| SM0035 | 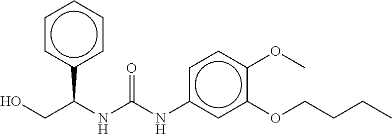 |
| SM0036 | 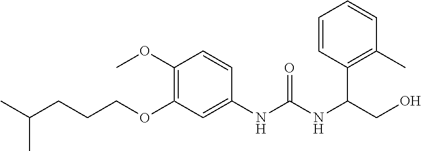 [SMILES: COc1ccc(NC(=O)NC(CO)c2ccccc2C)cc1OCCCC(C)C] |
| SM0037 | 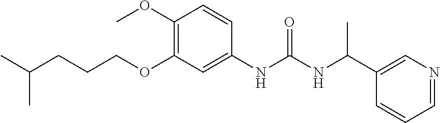 [SMILES: COc1ccc(NC(=O)NC(C)c2cccnc2)cc1OCCCC(C)C] |
| SM0038 | 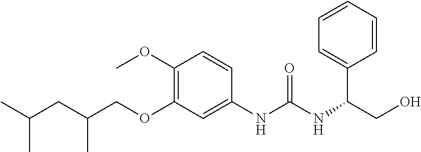 [SMILES: COc1ccc(NC(=O)N[C@@H](CO)c2ccccc2)cc1OCC(C)CC(C)C] |
| SM0039 | 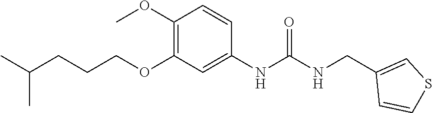 [SMILES: COc1ccc(NC(=O)NCc2ccsc2)cc1OCCCC(C)C] |
| SM0040 | 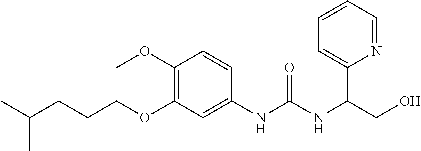 [SMILES: COc1ccc(NC(=O)NC(CO)c2ccccn2)cc1OCCCC(C)C] |
| SM0041 | 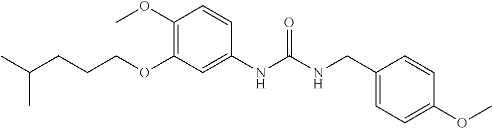 [SMILES: COc1ccc(CNC(=O)Nc2ccc(OC)c(OCCCC(C)C)c2)cc1] |
| SM0042 | 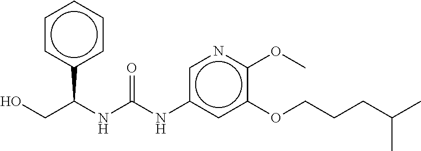 |
| SM0043 | 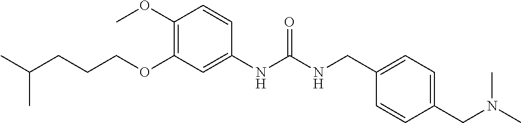 [SMILES: COc1ccc(NC(=O)NCc2ccc(CN(C)C)cc2)cc1OCCCC(C)C] |
| SM0044 | 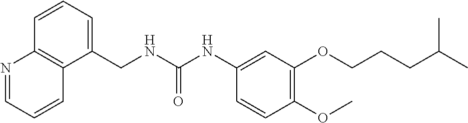 [SMILES: COc1ccc(NC(=O)NCc2cccc3ncccc23)cc1OCCCC(C)C] |
| SM0045 | 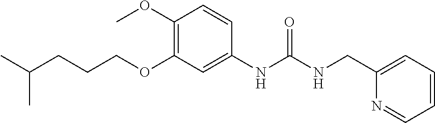 [SMILES: COc1ccc(NC(=O)NCc2ccccn2)cc1OCCCC(C)C] |
| SM0046 | 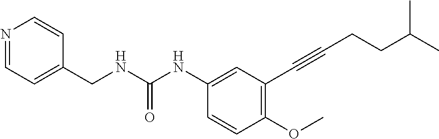 [SMILES: COc1ccc(NC(=O)NCc2ccncc2)cc1C#CCCC(C)C] |
| SM0047 | 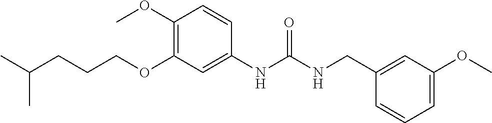 [SMILES: COc1cccc(CNC(=O)Nc2ccc(OC)c(OCCCC(C)C)c2)c1] |
| SM0048 | 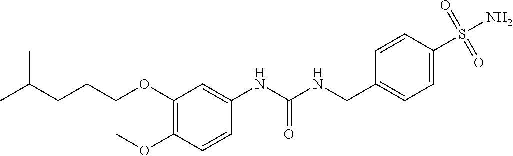 [SMILES: COc1ccc(NC(=O)NCc2ccc(S(N)(=O)=O)cc2)cc1OCCCC(C)C] |
| SM0049 | 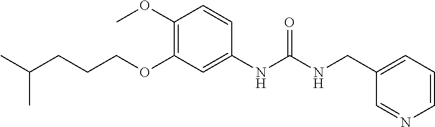 [SMILES: COc1ccc(NC(=O)NCc2cccnc2)cc1OCCCC(C)C] |
| SM0050 | 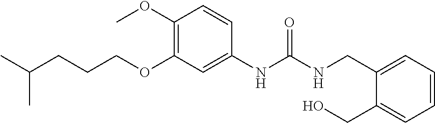 [SMILES: COc1ccc(NC(=O)NCc2ccccc2CO)cc1OCCCC(C)C] |
| SM0051 | 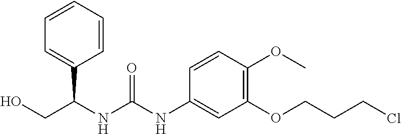 [SMILES: COc1ccc(NC(=O)N[C@@H](CO)c2ccccc2)cc1OCCCCl] |
| SM0052 | 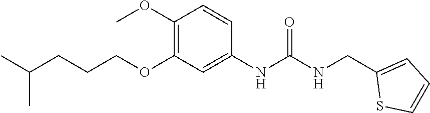 [SMILES: COc1ccc(NC(=O)NCc2cccs2)cc1OCCCC(C)C] |
| SM0053 | 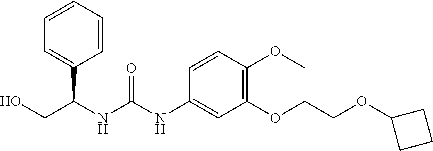 [SMILES: COc1ccc(NC(=O)N[C@@H](CO)c2ccccc2)cc1OCCOC1CCC1] |
| SM0054 | 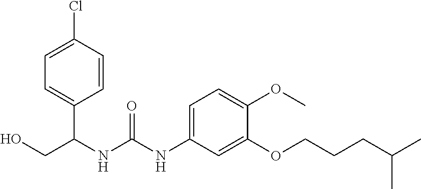 [SMILES: COc1ccc(NC(=O)NC(CO)c2ccc(Cl)cc2)cc1OCCCC(C)C] |
| SM0055 | 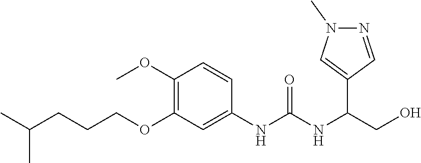 [SMILES: COc1ccc(NC(=O)NC(CO)c2cnn(C)c2)cc1OCCCC(C)C] |
| SM0056 | 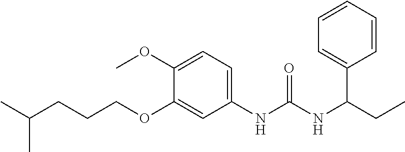 [SMILES: CCC(NC(=O)Nc1ccc(OC)c(OCCCC(C)C)c1)c1ccccc1] |
| SM0057 | 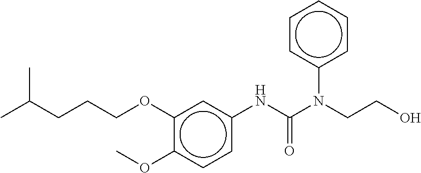 |
| SM0058 | 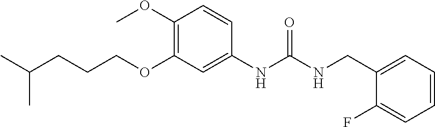 [SMILES: COc1ccc(NC(=O)NCc2ccccc2F)cc1OCCCC(C)C] |
| SM0059 | 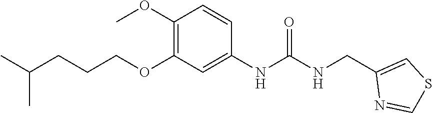 [SMILES: COc1ccc(NC(=O)NCc2cscn2)cc1OCCCC(C)C] |
| SM0060 | 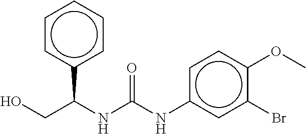 |
| SM0061 | 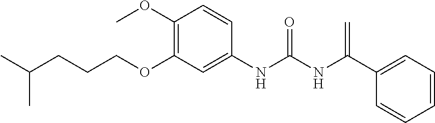 [SMILES: C=C(NC(=O)Nc1ccc(OC)c(OCCCC(C)C)c1)c1ccccc1] |
| SM0062 | 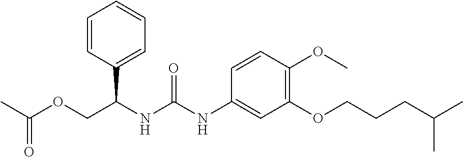 [SMILES: COc1ccc(NC(=O)N[C@@H](COC(C)=O)c2ccccc2)cc1OCCCC(C)C] |
| SM0063 | 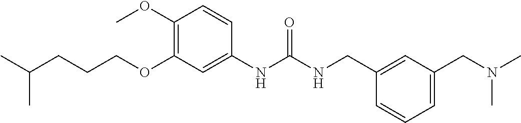 [SMILES: COc1ccc(NC(=O)NCc2cccc(CN(C)C)c2)cc1OCCCC(C)C] |
| SM0064 | 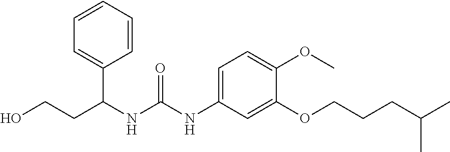 [SMILES: COc1ccc(NC(=O)NC(CCO)c2ccccc2)cc1OCCCC(C)C] |
| SM0065 | 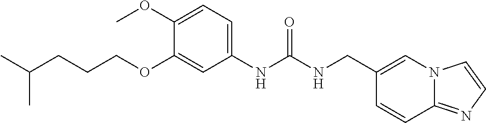 [SMILES: COc1ccc(NC(=O)NCc2ccc3nccn3c2)cc1OCCCC(C)C] |
| SM0066 | 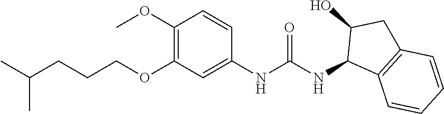 [SMILES: COc1ccc(NC(=O)N[C@@H]2c3ccccc3C[C@@H]2O)cc1OCCCC(C)C] |
| SM0067 | 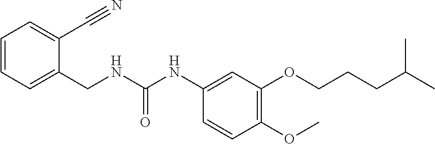 [SMILES: COc1ccc(NC(=O)NCc2ccccc2C#N)cc1OCCCC(C)C] |
| SM0068 | 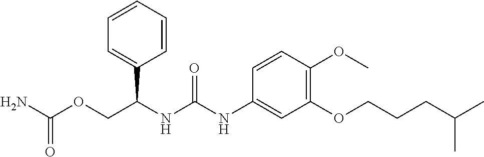 [SMILES: COc1ccc(NC(=O)N[C@@H](COC(N)=O)c2ccccc2)cc1OCCCC(C)C] |
| SM0069 | 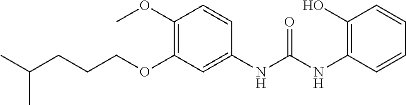 [SMILES: COc1ccc(NC(=O)Nc2ccccc2O)cc1OCCCC(C)C] |
| SM0070 | 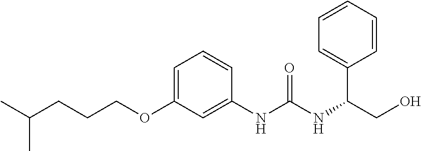 [SMILES: CC(C)CCCOc1cccc(NC(=O)N[C@@H](CO)c2ccccc2)c1] |
| SM0071 | 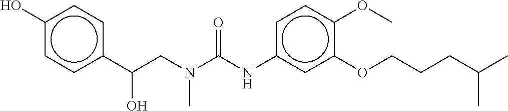 |
| SM0072 | 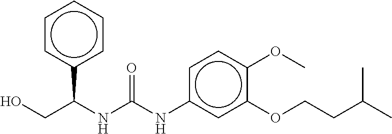 |
| SM0073 | 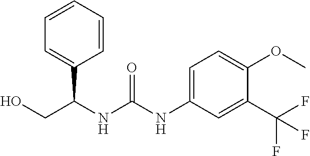 [SMILES: COc1ccc(NC(=O)N[C@@H](CO)c2ccccc2)cc1C(F)(F)F] |
| SM0074 | 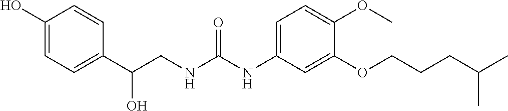 [SMILES: COc1ccc(NC(=O)NCC(O)c2ccc(O)cc2)cc1OCCCC(C)C] |
| SM0075 | 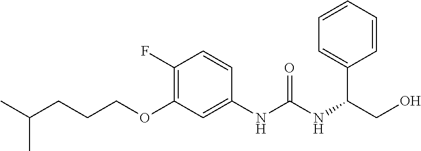 [SMILES: CC(C)CCCOc1cc(NC(=O)N[C@@H](CO)c2ccccc2)ccc1F] |
| SM0076 | 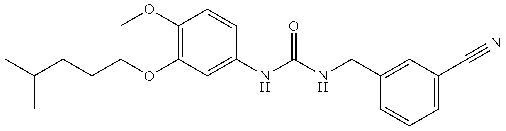 [SMILES: COc1ccc(NC(=O)NCc2cccc(C#N)c2)cc1OCCCC(C)C] |
| SM0077 | 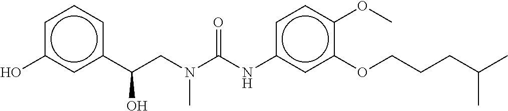 |
| SM0078 | 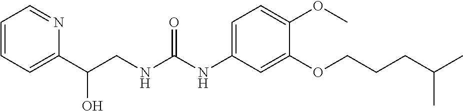 [SMILES: COc1ccc(NC(=O)NCC(O)c2ccccn2)cc1OCCCC(C)C] |
| SM0079 | 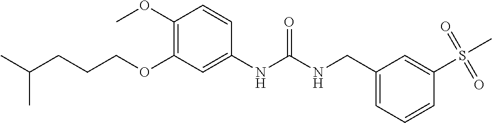 [SMILES: COc1ccc(NC(=O)NCc2cccc(S(C)(=O)=O)c2)cc1OCCCC(C)C] |
| SM0080 | 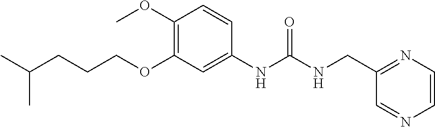 [SMILES: COc1ccc(NC(=O)NCc2cnccn2)cc1OCCCC(C)C] |
| SM0081 | 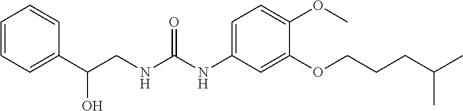 [SMILES: COc1ccc(NC(=O)NCC(O)c2ccccc2)cc1OCCCC(C)C] |
| SM0082 | 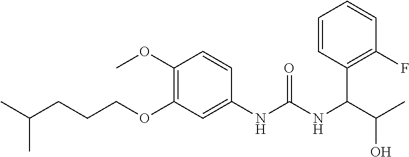 [SMILES: COc1ccc(NC(=O)NC(c2ccccc2F)C(C)O)cc1OCCCC(C)C] |
| SM0083 | 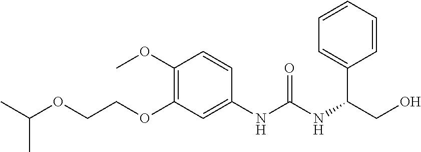 [SMILES: COc1ccc(NC(=O)N[C@@H](CO)c2ccccc2)cc1OCCOC(C)C] |
| SM0084 | 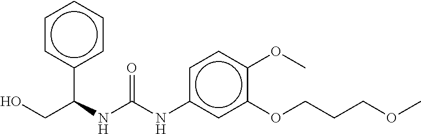 |
| SM0085 | 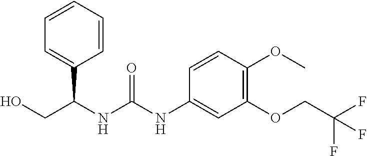 [SMILES: COc1ccc(NC(=O)N[C@@H](CO)c2ccccc2)cc1OCC(F)(F)F] |
| SM0086 | 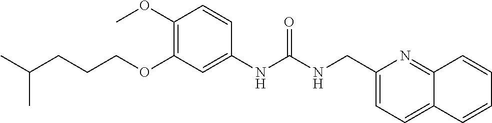 [SMILES: COc1ccc(NC(=O)NCc2ccc3ccccc3n2)cc1OCCCC(C)C] |
| SM0087 | 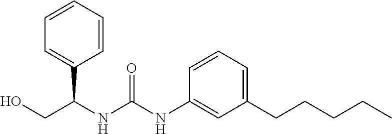 [SMILES: CCCCCc1cccc(NC(=O)N[C@@H](CO)c2ccccc2)c1] |
| SM0088 | 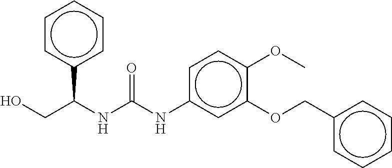 |
| SM0089 | 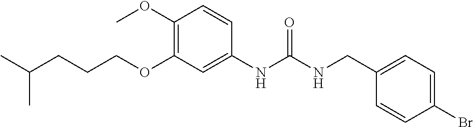 [SMILES: COc1ccc(NC(=O)NCc2ccc(Br)cc2)cc1OCCCC(C)C] |
| SM0090 | 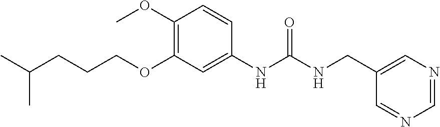 [SMILES: COc1ccc(NC(=O)NCc2cncnc2)cc1OCCCC(C)C] |
| SM0091 | 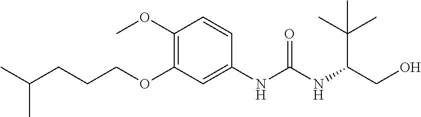 [SMILES: COc1ccc(NC(=O)N[C@@H](CO)C(C)(C)C)cc1OCCCC(C)C] |
| SM0092 | 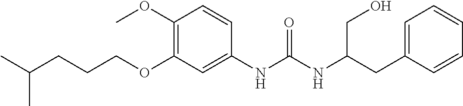 [SMILES: COc1ccc(NC(=O)NC(CO)Cc2ccccc2)cc1OCCCC(C)C] |
| SM0093 | 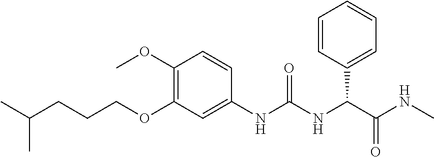 [SMILES: CNC(=O)[C@H](NC(=O)Nc1ccc(OC)c(OCCCC(C)C)c1)c1ccccc1] |
| SM0094 | 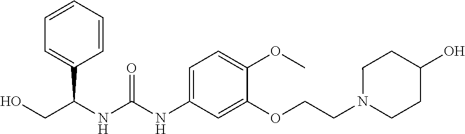 [SMILES: COc1ccc(NC(=O)N[C@@H](CO)c2ccccc2)cc1OCCN1CCC(O)CC1] |
| SM0095 | 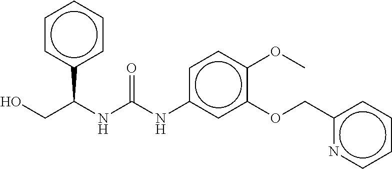 |
| SM0096 | 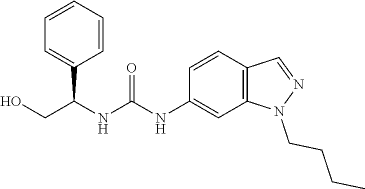 [SMILES: CCCCn1ncc2ccc(NC(=O)N[C@@H](CO)c3ccccc3)cc21] |
| SM0097 | 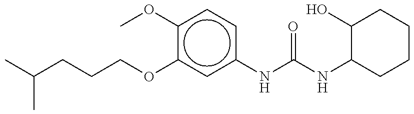 |
| SM0098 | 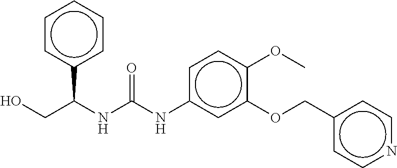 |
| SM0099 | 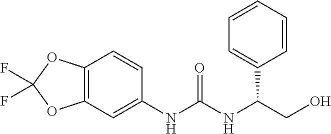 [SMILES: O=C(Nc1ccc2c(c1)OC(F)(F)O2)N[C@@H](CO)c1ccccc1] |
| SM0100 | 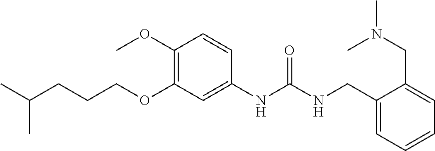 [SMILES: COc1ccc(NC(=O)NCc2ccccc2CN(C)C)cc1OCCCC(C)C] |
| SM0101 | 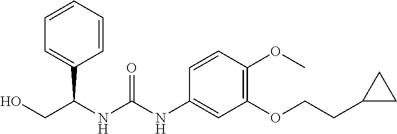 [SMILES: COc1ccc(NC(=O)N[C@@H](CO)c2ccccc2)cc1OCCC1CC1] |
| SM0102 | 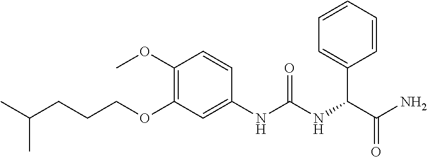 [SMILES: COc1ccc(NC(=O)N[C@@H](C(N)=O)c2ccccc2)cc1OCCCC(C)C] |
| SM0103 | 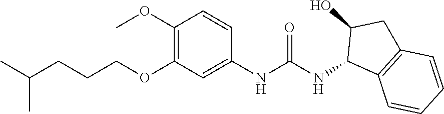 [SMILES: COc1ccc(NC(=O)N[C@H]2c3ccccc3C[C@@H]2O)cc1OCCCC(C)C] |
| SM0104 | 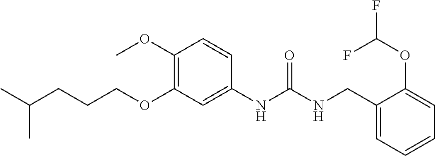 [SMILES: COc1ccc(NC(=O)NCc2ccccc2OC(F)F)cc1OCCCC(C)C] |
| SM0105 | 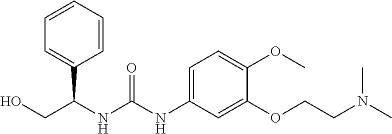 [SMILES: COc1ccc(NC(=O)N[C@@H](CO)c2ccccc2)cc1OCCN(C)C] |
| SM0106 | 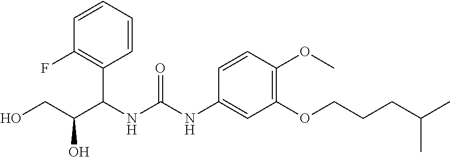 [SMILES: COc1ccc(NC(=O)NC(c2ccccc2F)[C@@H](O)CO)cc1OCCCC(C)C] |
| SM0107 | 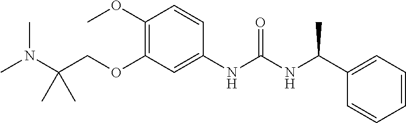 [SMILES: COc1ccc(NC(=O)N[C@@H](C)c2ccccc2)cc1OCC(C)(C)N(C)C] |
| SM0108 | 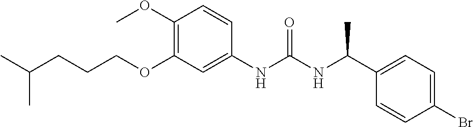 [SMILES: COc1ccc(NC(=O)N[C@@H](C)c2ccc(Br)cc2)cc1OCCCC(C)C] |
| SM0109 | 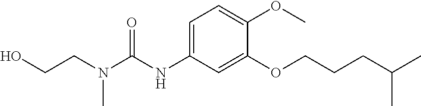 [SMILES: COc1ccc(NC(=O)N(C)CCO)cc1OCCCC(C)C] |
| SM0110 | 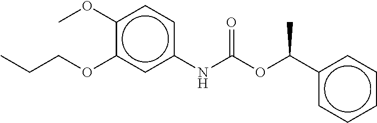 |
| SM0111 | 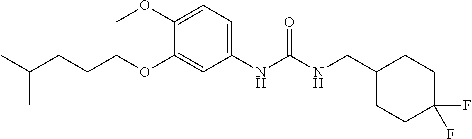 [SMILES: COc1ccc(NC(=O)NCC2CCC(F)(F)CC2)cc1OCCCC(C)C] |
| SM0112 | 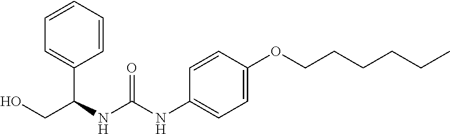 [SMILES: CCCCCCOc1ccc(NC(=O)N[C@@H](CO)c2ccccc2)cc1] |
| SM0113 | 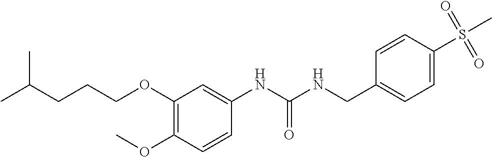 [SMILES: COc1ccc(NC(=O)NCc2ccc(S(C)(=O)=O)cc2)cc1OCCCC(C)C] |
| SM0114 | 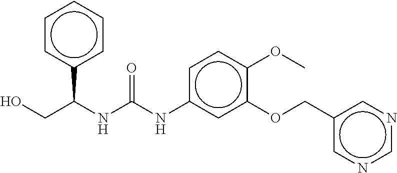 |
| SM0115 | 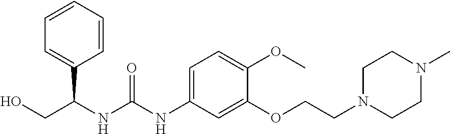 [SMILES: COc1ccc(NC(=O)N[C@@H](CO)c2ccccc2)cc1OCCN1CCN(C)CC1] |
| SM0116 | 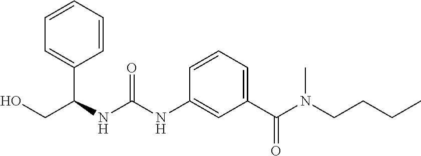 [SMILES: CCCCN(C)C(=O)c1cccc(NC(=O)N[C@@H](CO)c2ccccc2)c1] |
| SM0117 | 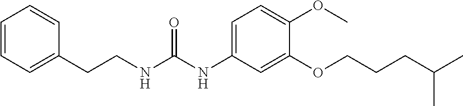 [SMILES: COc1ccc(NC(=O)NCCc2ccccc2)cc1OCCCC(C)C] |
| SM0118 | 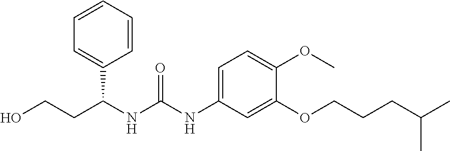 [SMILES: COc1ccc(NC(=O)N[C@H](CCO)c2ccccc2)cc1OCCCC(C)C] |
| SM0119 | 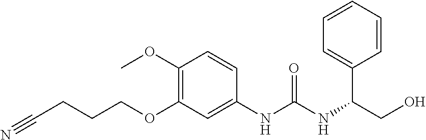 [SMILES: COc1ccc(NC(=O)N[C@@H](CO)c2ccccc2)cc1OCCCC#N] |
| SM0120 | 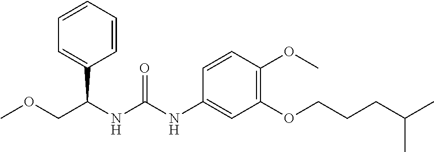 [SMILES: COC[C@H](NC(=O)Nc1ccc(OC)c(OCCCC(C)C)c1)c1ccccc1] |
| SM0121 | 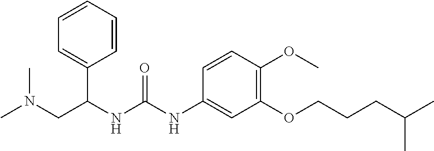 [SMILES: COc1ccc(NC(=O)NC(CN(C)C)c2ccccc2)cc1OCCCC(C)C] |
| SM0200 | 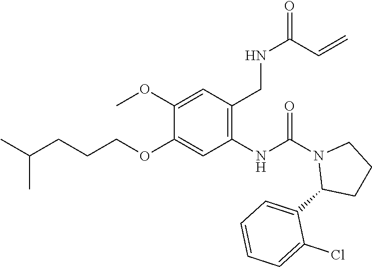 [SMILES: C=CC(=O)NCc1cc(OC)c(OCCCC(C)C)cc1NC(=O)N1CCC[C@@H]1c1ccccc1Cl] |
| SM0201 | 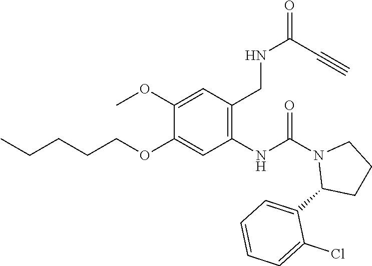 [SMILES: C#CC(=O)NCc1cc(OC)c(OCCCCC)cc1NC(=O)N1CCC[C@@H]1c1ccccc1Cl] |
| SM0202 | 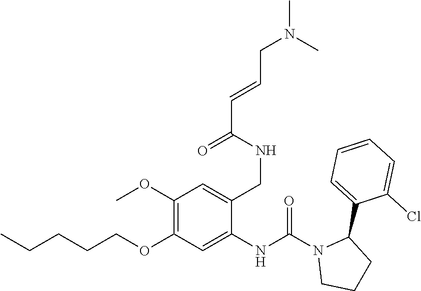 [SMILES: CCCCCOc1cc(NC(=O)N2CCC[C@@H]2c2ccccc2Cl)c(CNC(=O)/C=C/CN(C)C)cc1OC] |
| SM0203 | 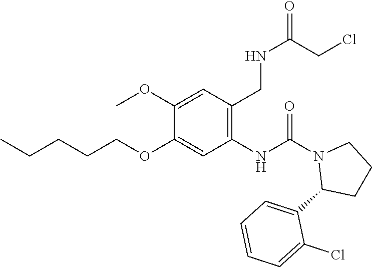 [SMILES: CCCCCOc1cc(NC(=O)N2CCC[C@@H]2c2ccccc2Cl)c(CNC(=O)CCl)cc1OC] |
| SM0204 | 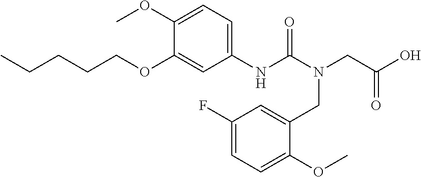 [SMILES: CCCCCOc1cc(NC(=O)N(CC(=O)O)Cc2cc(F)ccc2OC)ccc1OC] |
| SM0205 | 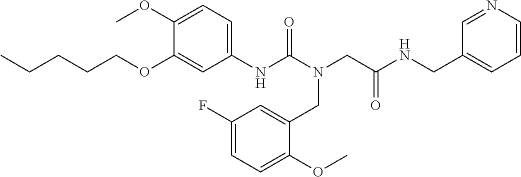 [SMILES: CCCCCOc1cc(NC(=O)N(CC(=O)NCc2cccnc2)Cc2cc(F)ccc2OC)ccc1OC] |
| SM0206 | 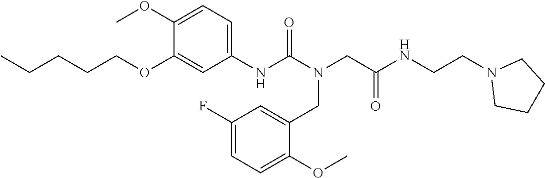 [SMILES: CCCCCOc1cc(NC(=O)N(CC(=O)NCCN2CCCC2)Cc2cc(F)ccc2OC)ccc1OC] |
| SM0207 | 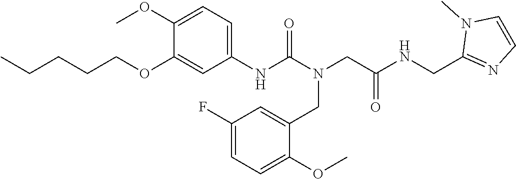 [SMILES: CCCCCOc1cc(NC(=O)N(CC(=O)NCc2nccn2C)Cc2cc(F)ccc2OC)ccc1OC] |
| SM0208 | 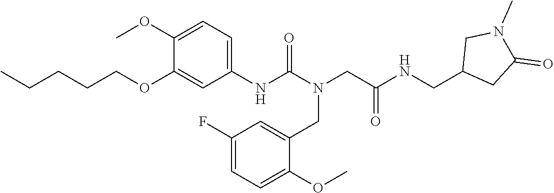 [SMILES: CCCCCOc1cc(NC(=O)N(CC(=O)NCC2CC(=O)N(C)C2)Cc2cc(F)ccc2OC)ccc1OC] |
| SM0209 | 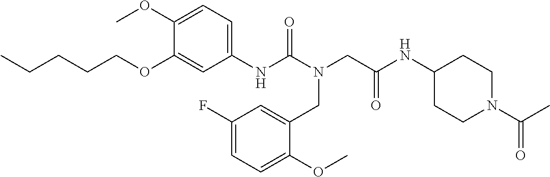 [SMILES: CCCCCOc1cc(NC(=O)N(CC(=O)NC2CCN(C(C)=O)CC2)Cc2cc(F)ccc2OC)ccc1OC] |
| SM0210 | 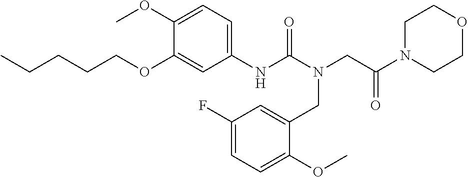 [SMILES: CCCCCOc1cc(NC(=O)N(CC(=O)N2CCOCC2)Cc2cc(F)ccc2OC)ccc1OC] |
| SM0211 | 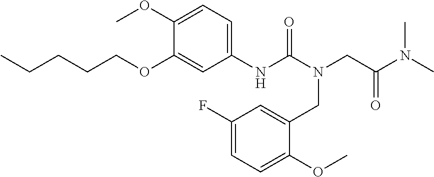 [SMILES: CCCCCOc1cc(NC(=O)N(CC(=O)N(C)C)Cc2cc(F)ccc2OC)ccc1OC] |
| SM0212 | 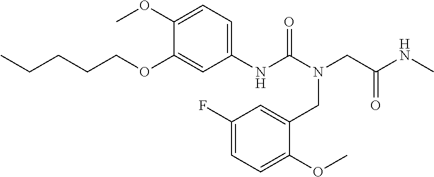 [SMILES: CCCCCOc1cc(NC(=O)N(CC(=O)NC)Cc2cc(F)ccc2OC)ccc1OC] |
| SM0213 | 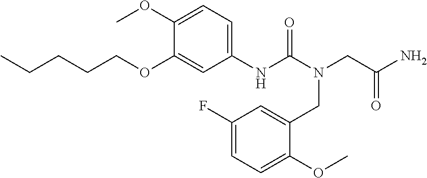 [SMILES: CCCCCOc1cc(NC(=O)N(CC(N)=O)Cc2cc(F)ccc2OC)ccc1OC] |
| SM0214 | 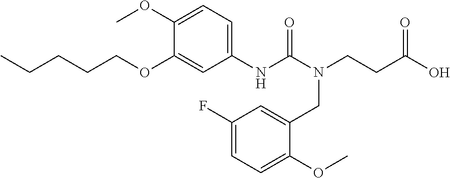 [SMILES: CCCCCOc1cc(NC(=O)N(CCC(=O)O)Cc2cc(F)ccc2OC)ccc1OC] |
| SM0215 | 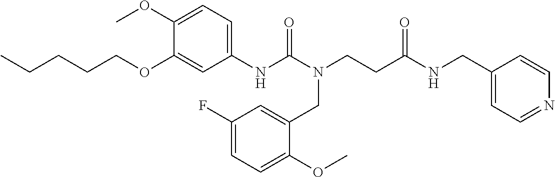 [SMILES: CCCCCOc1cc(NC(=O)N(CCC(=O)NCc2ccncc2)Cc2cc(F)ccc2OC)ccc1OC] |
| SM0216 | 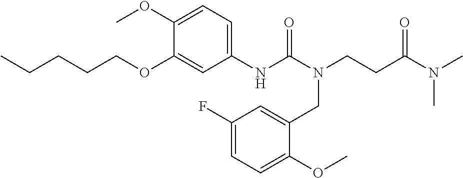 [SMILES: CCCCCOc1cc(NC(=O)N(CCC(=O)N(C)C)Cc2cc(F)ccc2OC)ccc1OC] |
| SM0217 | 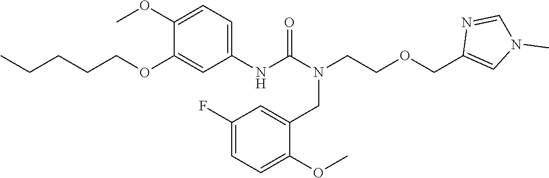 [SMILES: CCCCCOc1cc(NC(=O)N(CCOCc2cn(C)cn2)Cc2cc(F)ccc2OC)ccc1OC] |
| SM0218 | 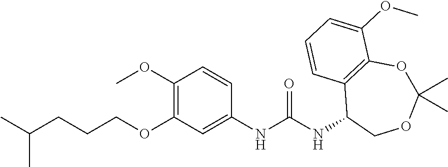 [SMILES: COc1ccc(NC(=O)N[C@H]2COC(C)(C)Oc3c(OC)cccc32)cc1OCCCC(C)C] |
| SM0219 | 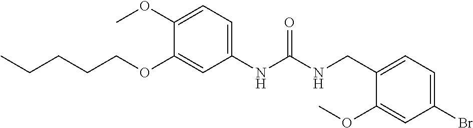 [SMILES: CCCCCOc1cc(NC(=O)NCc2ccc(Br)cc2OC)ccc1OC] |
| C5INH- 0294 | 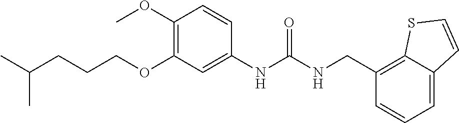 [SMILES: COc1ccc(NC(=O)NCc2cccc3ccsc23)cc1OCCCC(C)C] |
| C5INH- 0296 | 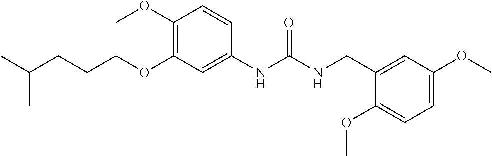 [SMILES: COc1ccc(OC)c(CNC(=O)Nc2ccc(OC)c(OCCCC(C)C)c2)c1] |
| C5INH- 0298 | 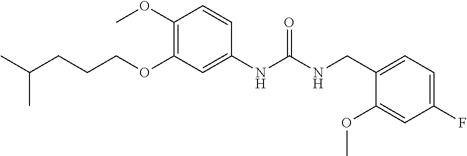 [SMILES: COc1cc(F)ccc1CNC(=O)Nc1ccc(OC)c(OCCCC(C)C)c1] |
| C5INH- 0303 | 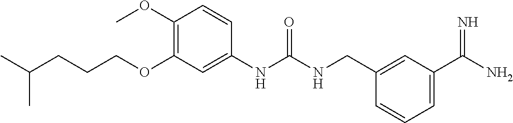 [SMILES: COc1ccc(NC(=O)NCc2cccc(C(=N)N)c2)cc1OCCCC(C)C] |
| C5INH- 0310 | 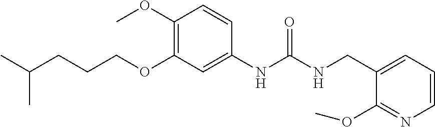 [SMILES: COc1ccc(NC(=O)NCc2cccnc2OC)cc1OCCCC(C)C] |
| C5INH- 0311 | 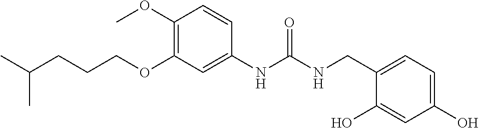 [SMILES: COc1ccc(NC(=O)NCc2ccc(O)cc2O)cc1OCCCC(C)C] |
| C5INH- 0315 | 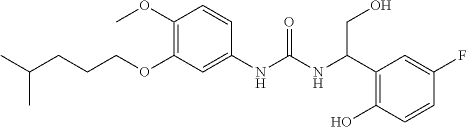 [SMILES: COc1ccc(NC(=O)NC(CO)c2cc(F)ccc2O)cc1OCCCC(C)C] |
| C5INH- 0316 | 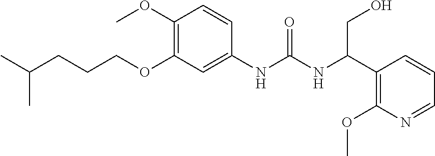 [SMILES: COc1ccc(NC(=O)NC(CO)c2cccnc2OC)cc1OCCCC(C)C] |
| C5INH- 0317 | 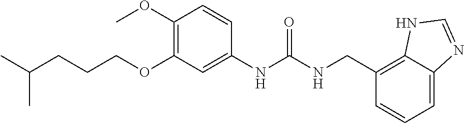 [SMILES: COc1ccc(NC(=O)NCc2cccc3nc[nH]c23)cc1OCCCC(C)C] |
| C5INH- 0318 | 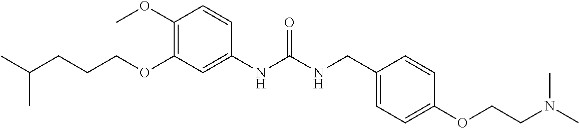 [SMILES: COc1ccc(NC(=O)NCc2ccc(OCCN(C)C)cc2)cc1OCCCC(C)C] |
| C5INH- 0319 | 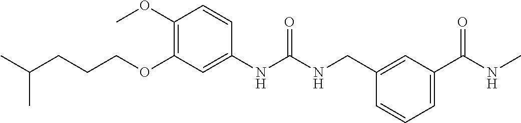 [SMILES: CNC(=O)c1cccc(CNC(=O)Nc2ccc(OC)c(OCCCC(C)C)c2)c1] |
| C5INH- 0321 | 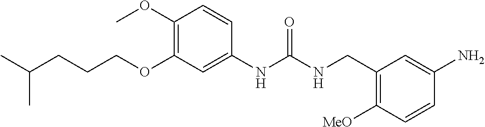 [SMILES: COc1ccc(N)cc1CNC(=O)Nc1ccc(OC)c(OCCCC(C)C)c1] |
| C5INH- 0323 | 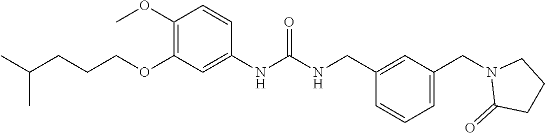 [SMILES: COc1ccc(NC(=O)NCc2cccc(CN3CCCC3=O)c2)cc1OCCCC(C)C] |
| C5INH- 0324 | 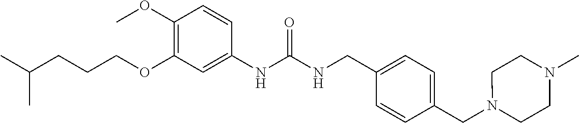 [SMILES: COc1ccc(NC(=O)NCc2ccc(CN3CCN(C)CC3)cc2)cc1OCCCC(C)C] |
| C5INH- 0326 | 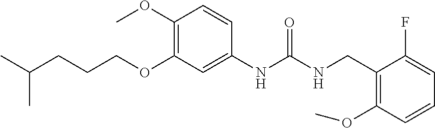 [SMILES: COc1ccc(NC(=O)NCc2c(F)cccc2OC)cc1OCCCC(C)C] |
| C5INH- 0329 | 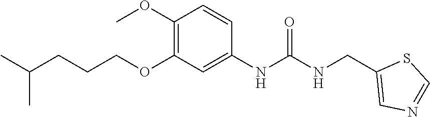 [SMILES: COc1ccc(NC(=O)NCc2cncs2)cc1OCCCC(C)C] |
| C5INH- 0330 | 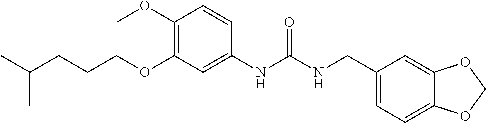 [SMILES: COc1ccc(NC(=O)NCc2ccc3c(c2)OCO3)cc1OCCCC(C)C] |
| C5INH- 0333 | 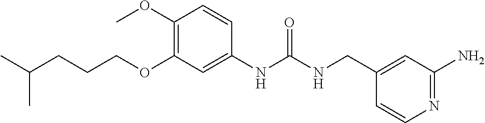 [SMILES: COc1ccc(NC(=O)NCc2ccnc(N)c2)cc1OCCCC(C)C] |
| C5INH- 0335 | 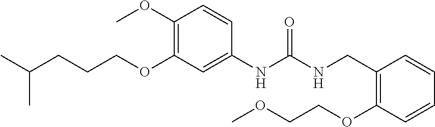 [SMILES: COCCOc1ccccc1CNC(=O)Nc1ccc(OC)c(OCCCC(C)C)c1] |
| C5INH- 0336 | 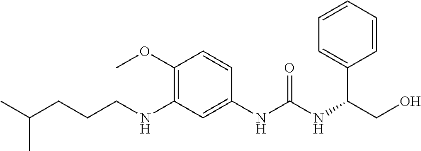 [SMILES: COc1ccc(NC(=O)N[C@@H](CO)c2ccccc2)cc1NCCCC(C)C] |
| C5INH- 0338 | 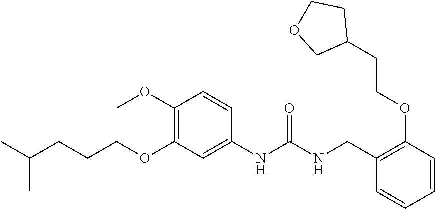 [SMILES: COc1ccc(NC(=O)NCc2ccccc2OCCC2CCOC2)cc1OCCCC(C)C] |
| C5INH- 0339 | 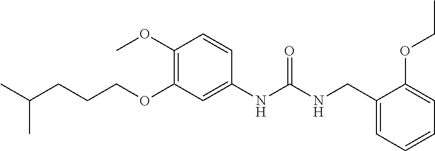 [SMILES: CCOc1ccccc1CNC(=O)Nc1ccc(OC)c(OCCCC(C)C)c1] |
| C5INH- 0340 | 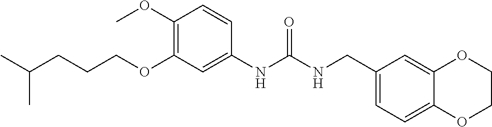 [SMILES: COc1ccc(NC(=O)NCc2ccc3c(c2)OCCO3)cc1OCCCC(C)C] |
| C5INH- 0342 | 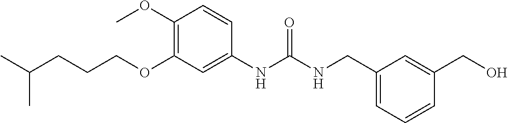 [SMILES: COc1ccc(NC(=O)NCc2cccc(CO)c2)cc1OCCCC(C)C] |
| C5INH- 0343 | 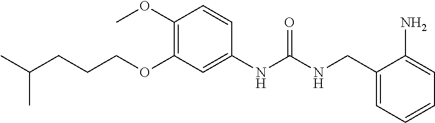 [SMILES: COc1ccc(NC(=O)NCc2ccccc2N)cc1OCCCC(C)C] |
| C5INH- 0348 | 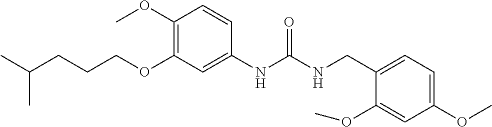 [SMILES: COc1ccc(CNC(=O)Nc2ccc(OC)c(OCCCC(C)C)c2)c(OC)c1] |
| C5INH- 0349 | 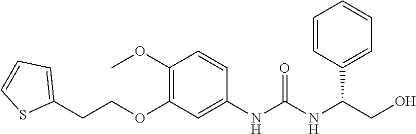 [SMILES: COc1ccc(NC(=O)N[C@@H](CO)c2ccccc2)cc1OCCc1cccs1] |
| C5INH- 0350 | 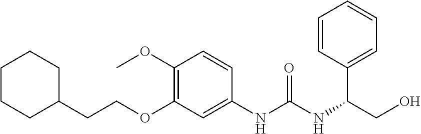 [SMILES: COc1ccc(NC(=O)N[C@@H](CO)c2ccccc2)cc1OCCC1CCCCC1] |
| C5INH- 0352 | 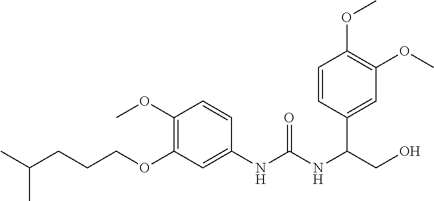 [SMILES: COc1ccc(C(CO)NC(=O)Nc2ccc(OC)c(OCCCC(C)C)c2)cc1OC] |
| C5INH- 0353 | 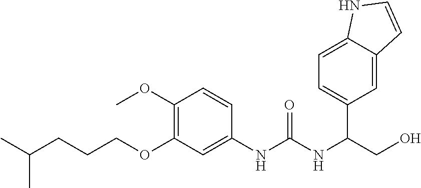 [SMILES: COc1ccc(NC(=O)NC(CO)c2ccc3[nH]ccc3c2)cc1OCCCC(C)C] |
| C5INH- 0355 | 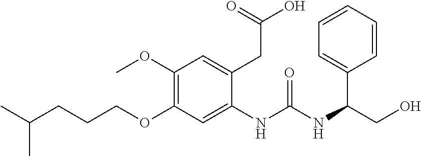 [SMILES: COc1cc(CC(=O)O)c(NC(=O)N[C@H](CO)c2ccccc2)cc1OCCCC(C)C] |
| C5INH- 0356 | 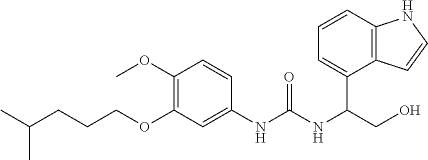 [SMILES: COc1ccc(NC(=O)NC(CO)c2cccc3[nH]ccc23)cc1OCCCC(C)C] |
| C5INH- 0357 | 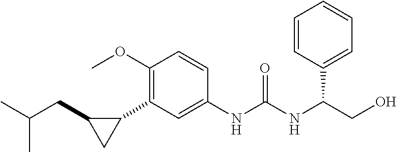 |
| C6INH- 0361 | 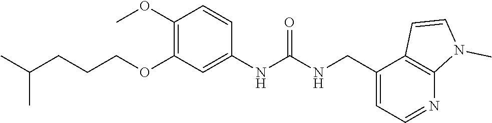 [SMILES: COc1ccc(NC(=O)NCc2ccnc3c2ccn3C)cc1OCCCC(C)C] |
| C5INH- 0366 | 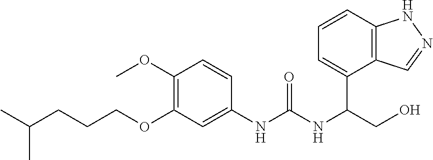 [SMILES: COc1ccc(NC(=O)NC(CO)c2cccc3[nH]ncc23)cc1OCCCC(C)C] |
| C5INH- 0367 | 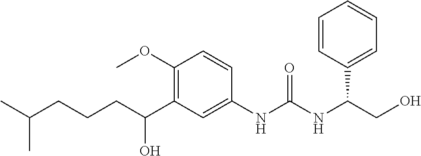 [SMILES: COc1ccc(NC(=O)N[C@@H](CO)c2ccccc2)cc1C(O)CCCC(C)C] |
| C5INH- 0369 | 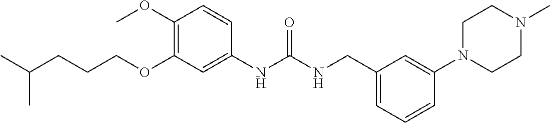 [SMILES: COc1ccc(NC(=O)NCc2cccc(N3CCN(C)CC3)c2)cc1OCCCC(C)C] |
| C5INH- 0370 | 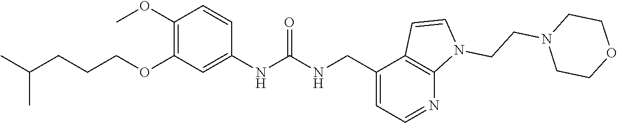 [SMILES: COc1ccc(NC(=O)NCc2ccnc3c2ccn3CCN2CCOCC2)cc1OCCCC(C)C] |
| C5INH- 0371 | 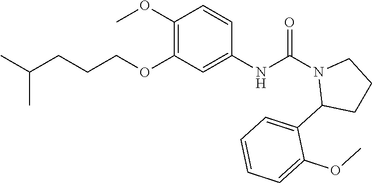 [SMILES: COc1ccc(NC(=O)N2CCCC2c2ccccc2OC)cc1OCCCC(C)C] |
| C5INH- 0372 | 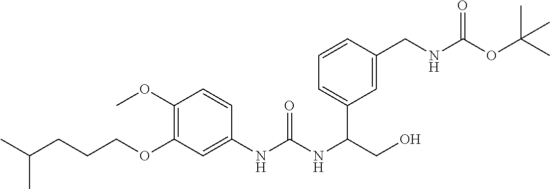 [SMILES: COc1ccc(NC(=O)NC(CO)c2cccc(CNC(=O)OC(C)(C)C)c2)cc1OCCCC(C)C] |
| C5INH- 0373 | 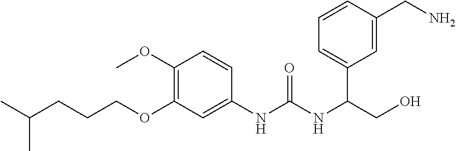 [SMILES: COc1ccc(NC(=O)NC(CO)c2cccc(CN)c2)cc1OCCCC(C)C] |
| C5INH- 0377 | 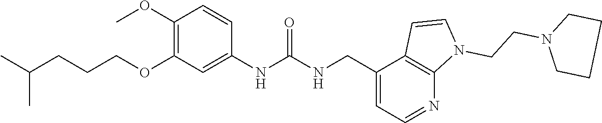 [SMILES: COc1ccc(NC(=O)NCc2ccnc3c2ccn3CCN2CCCC2)cc1OCCCC(C)C] |
| C5INH- 0379 | 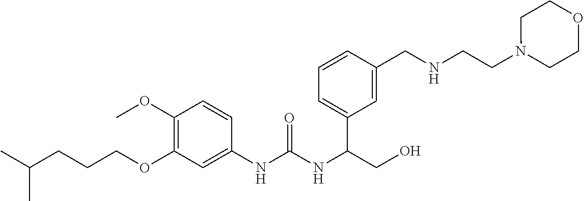 [SMILES: COc1ccc(NC(=O)NC(CO)c2cccc(CNCCN3CCOCC3)c2)cc1OCCCC(C)C] |
| C5INH- 0381 | 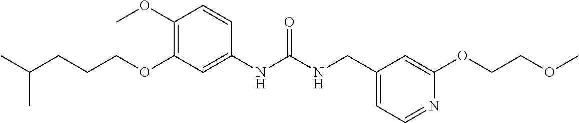 [SMILES: COCCOc1cc(CNC(=O)Nc2ccc(OC)c(OCCCC(C)C)c2)ccn1] |
| C5INH- 0382 | 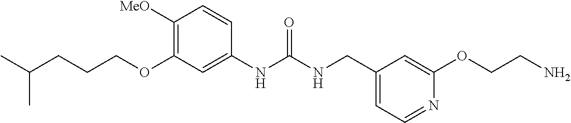 [SMILES: COc1ccc(NC(=O)NCc2ccnc(OCCN)c2)cc1OCCCC(C)C] |
| C5INH- 0383 | 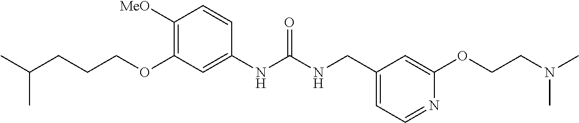 [SMILES: COc1ccc(NC(=O)NCc2ccnc(OCCN(C)C)c2)cc1OCCCC(C)C] |
| C5INH- 0384 | 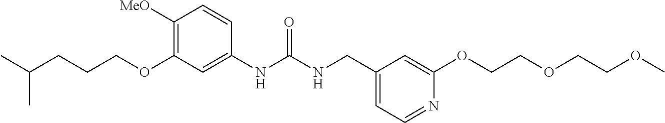 [SMILES: COCCOCCOc1cc(CNC(=O)Nc2ccc(OC)c(OCCCC(C)C)c2)ccn1] |
| C5INH- 0385 | 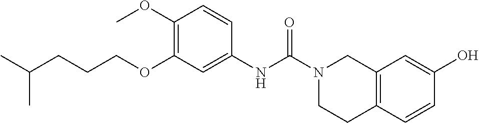 [SMILES: COc1ccc(NC(=O)N2CCc3ccc(O)cc3C2)cc1OCCCC(C)C] |
| C5INH- 0387 | 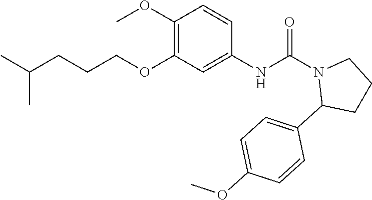 [SMILES: COc1ccc(C2CCCN2C(=O)Nc2ccc(OC)c(OCCCC(C)C)c2)cc1] |
| C5INH- 0388 | 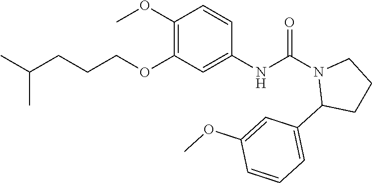 [SMILES: COc1cccc(C2CCCN2C(=O)Nc2ccc(OC)c(OCCCC(C)C)c2)c1] |
| C5INH- 0389 | 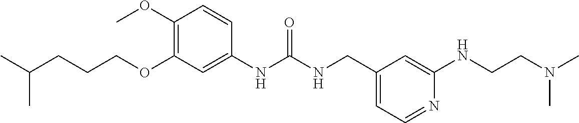 [SMILES: COc1ccc(NC(=O)NCc2ccnc(NCCN(C)C)c2)cc1OCCCC(C)C] |
| C5INH- 0390 | 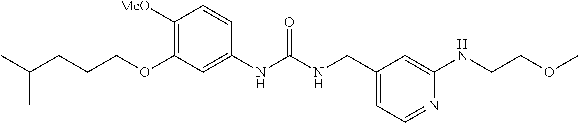 [SMILES: COCCNc1cc(CNC(=O)Nc2ccc(OC)c(OCCCC(C)C)c2)ccn1] |
| C5INH- 0391 | 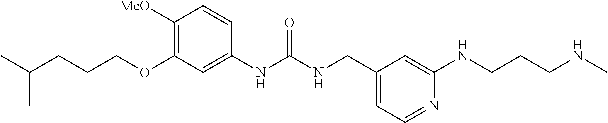 [SMILES: CNCCCNc1cc(CNC(=O)Nc2ccc(OC)c(OCCCC(C)C)c2)ccn1] |
| C5INH- 0395 | 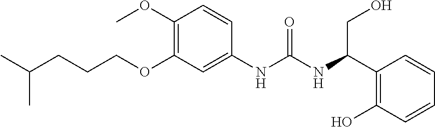 [SMILES: COc1ccc(NC(=O)N[C@@H](CO)c2ccccc2O)cc1OCCCC(C)C] |
| C5INH- 0396 | 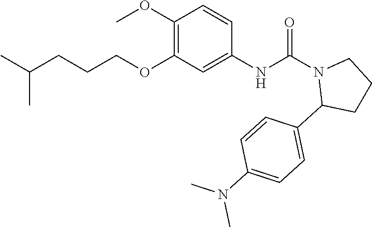 [SMILES: COc1ccc(NC(=O)N2CCCC2c2ccc(N(C)C)cc2)cc1OCCCC(C)C] |
| C5INH- 0397 | 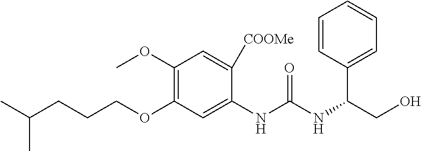 [SMILES: COC(=O)c1cc(OC)c(OCCCC(C)C)cc1NC(=O)N[C@@H](CO)c1ccccc1] |
| C5INH- 0398 | 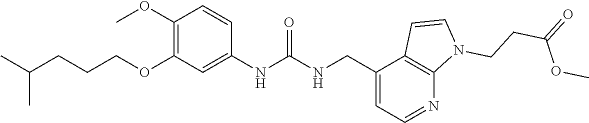 [SMILES: COC(=O)CCn1ccc2c(CNC(=O)Nc3ccc(OC)c(OCCCC(C)C)c3)ccnc21] |
| C5INH- 0399 | 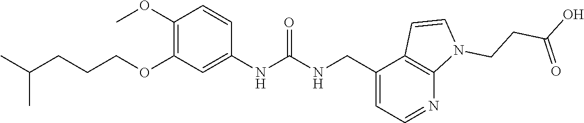 [SMILES: COc1ccc(NC(=O)NCc2ccnc3c2ccn3CCC(=O)O)cc1OCCCC(C)C] |
| C5INH- 0401 | 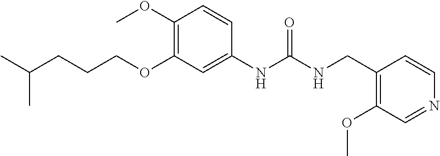 [SMILES: COc1cnccc1CNC(=O)Nc1ccc(OC)c(OCCCC(C)C)c1] |
| C5INH- 0402 | 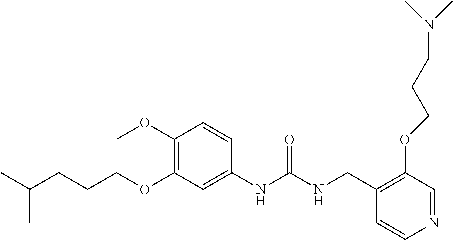 [SMILES: COc1ccc(NC(=O)NCc2ccncc2OCCCN(C)C)cc1OCCCC(C)C] |
| C5INH- 0403 | 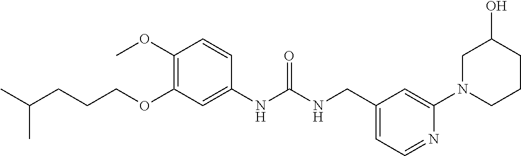 [SMILES: COc1ccc(NC(=O)NCc2ccnc(N3CCCC(O)C3)c2)cc1OCCCC(C)C] |
| C5INH- 0406 | 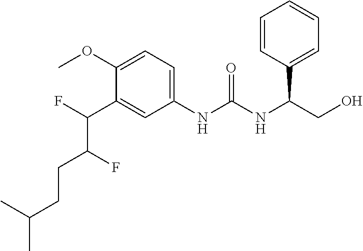 [SMILES: COc1ccc(NC(=O)N[C@H](CO)c2ccccc2)cc1C(F)C(F)CCC(C)C] |
| C5INH- 0409 | 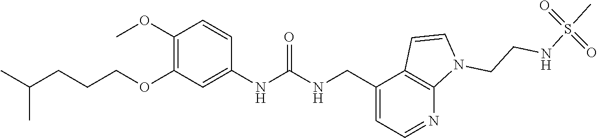 [SMILES: COc1ccc(NC(=O)NCc2ccnc3c2ccn3CCNS(C)(=O)=O)cc1OCCCC(C)C] |
| C5INH- 0410 | 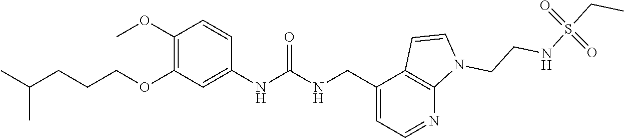 [SMILES: CCS(=O)(=O)NCCn1ccc2c(CNC(=O)Nc3ccc(OC)c(OCCCC(C)C)c3)ccnc21] |
| C5INH- 0411 | 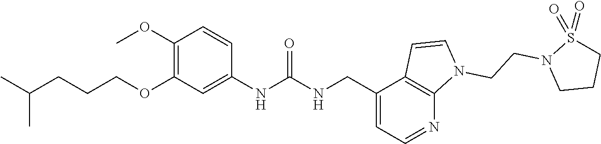 [SMILES: COc1ccc(NC(=O)NCc2ccnc3c2ccn3CCN2CCCS2(=O)=O)cc1OCCCC(C)C] |
| C5INH- 0414 | 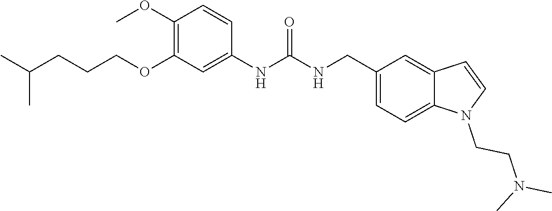 [SMILES: COc1ccc(NC(=O)NCc2ccc3c(ccn3CCN(C)C)c2)cc1OCCCC(C)C] |
| C5INH- 0417 | 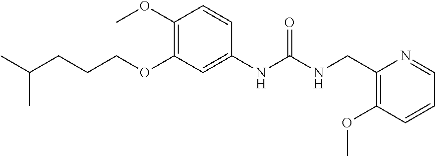 [SMILES: COc1ccc(NC(=O)NCc2ncccc2OC)cc1OCCCC(C)C] |
| C5INH- 0420 | 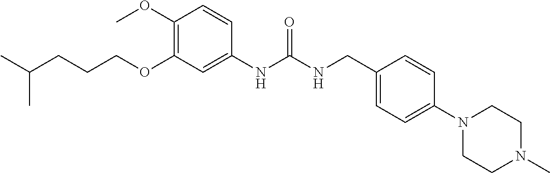 [SMILES: COc1ccc(NC(=O)NCc2ccc(N3CCN(C)CC3)cc2)cc1OCCCC(C)C] |
| C5INH- 0421 | 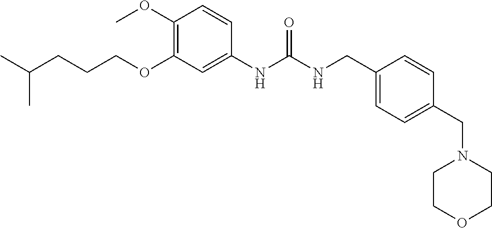 [SMILES: COc1ccc(NC(=O)NCc2ccc(CN3CCOCC3)cc2)cc1OCCCC(C)C] |
| C5INH- 0422 | 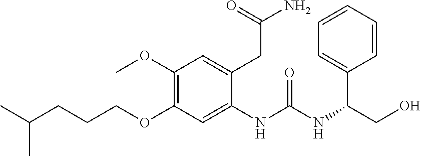 [SMILES: COc1cc(CC(N)=O)c(NC(=O)N[C@@H](CO)c2ccccc2)cc1OCCCC(C)C] |
| C5INH- 0425 | 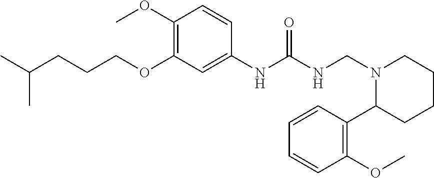 [SMILES: COc1ccc(NC(=O)NCN2CCCCC2c2ccccc2OC)cc1OCCCC(C)C] |
| C5INH- 0428 | 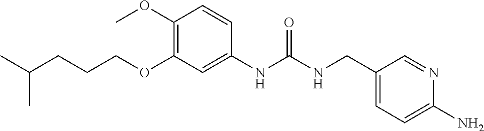 [SMILES: COc1ccc(NC(=O)NCc2ccc(N)nc2)cc1OCCCC(C)C] |
| C5INH- 0431 | 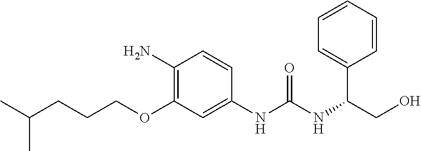 [SMILES: CC(C)CCCOc1cc(NC(=O)N[C@@H](CO)c2ccccc2)ccc1N] |
| C5INH- 0432 | 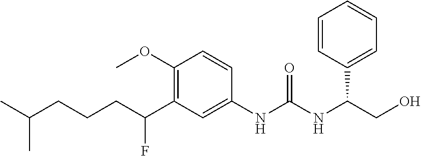 [SMILES: COc1ccc(NC(=O)N[C@@H](CO)c2ccccc2)cc1C(F)CCCC(C)C] |
| C5INH- 0436 | 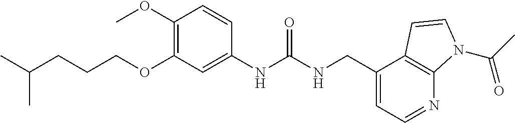 [SMILES: COc1ccc(NC(=O)NCc2ccnc3c2ccn3C(C)=O)cc1OCCCC(C)C] |
| C5INH- 0437 | 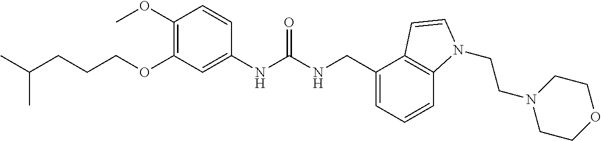 [SMILES: COc1ccc(NC(=O)NCc2cccc3c2ccn3CCN2CCOCC2)cc1OCCCC(C)C] |
| C5INH- 0438 | 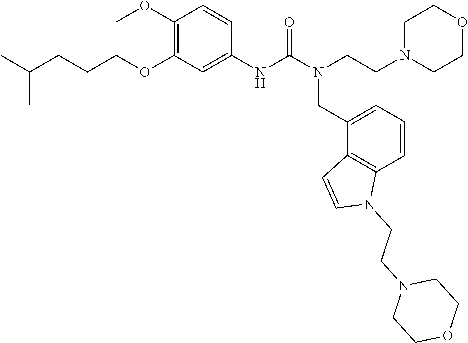 [SMILES: COc1ccc(NC(=O)N(CCN2CCOCC2)Cc2cccc3c2ccn3CCN2CCOCC2)cc1OCCCC(C)C] |
| C5INH- 0440 | 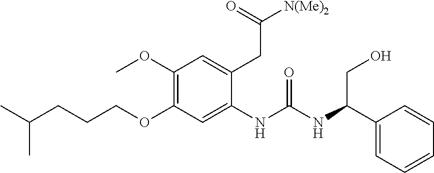 |
| C5INH- 0443 | 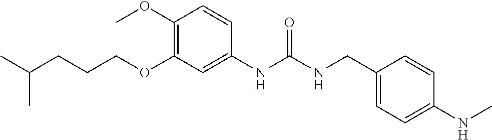 [SMILES: CNc1ccc(CNC(=O)Nc2ccc(OC)c(OCCCC(C)C)c2)cc1] |
| C5INH- 0446 | 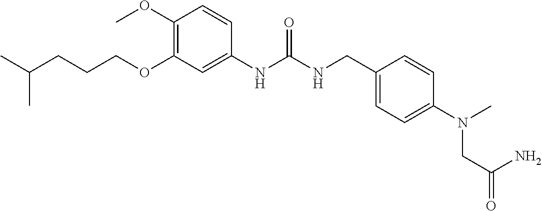 [SMILES: COc1ccc(NC(=O)NCc2ccc(N(C)CC(N)=O)cc2)cc1OCCCC(C)C] |
| C5INH- 0447 | 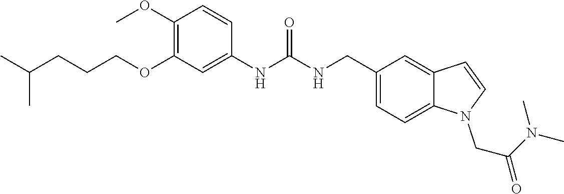 [SMILES: COc1ccc(NC(=O)NCc2ccc3c(ccn3CC(=O)N(C)C)c2)cc1OCCCC(C)C] |
| C5INH- 0448 | 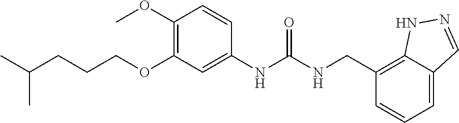 [SMILES: COc1ccc(NC(=O)NCc2cccc3cn[nH]c23)cc1OCCCC(C)C] |
| C5INH- 0450 | 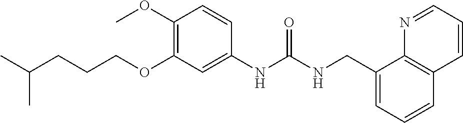 [SMILES: COc1ccc(NC(=O)NCc2cccc3cccnc23)cc1OCCCC(C)C] |
| C5INH- 0452 | 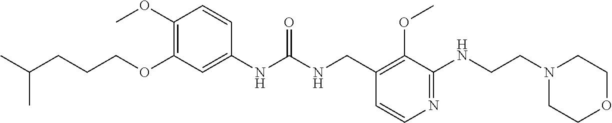 [SMILES: COc1ccc(NC(=O)NCc2ccnc(NCCN3CCOCC3)c2OC)cc1OCCCC(C)C] |
| C5INH- 0453 | 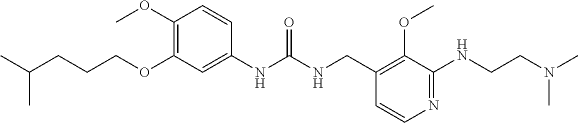 [SMILES: COc1ccc(NC(=O)NCc2ccnc(NCCN(C)C)c2OC)cc1OCCCC(C)C] |
| C5INH- 0454 | 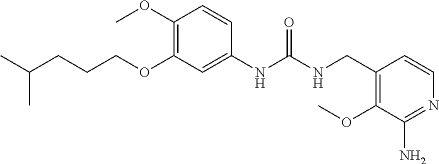 [SMILES: COc1ccc(NC(=O)NCc2ccnc(N)c2OC)cc1OCCCC(C)C] |
| C5INH- 0456 | 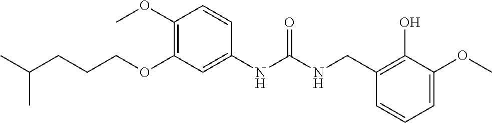 [SMILES: COc1ccc(NC(=O)NCc2cccc(OC)c2O)cc1OCCCC(C)C] |
| C5INH- 0458 | 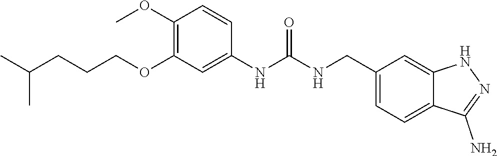 [SMILES: COc1ccc(NC(=O)NCc2ccc3c(N)n[nH]c3c2)cc1OCCCC(C)C] |
| C5INH- 0460 | 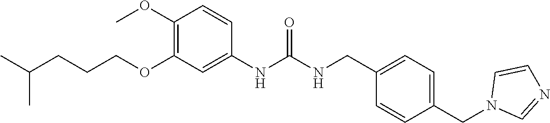 [SMILES: COc1ccc(NC(=O)NCc2ccc(Cn3ccnc3)cc2)cc1OCCCC(C)C] |
| C5INH- 0462 | 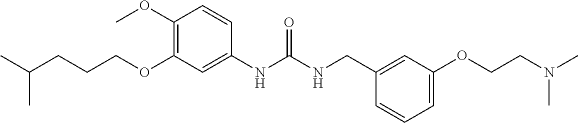 [SMILES: COc1ccc(NC(=O)NCc2cccc(OCCN(C)C)c2)cc1OCCCC(C)C] |
| C5INH- 0463 | 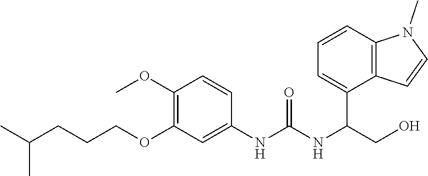 [SMILES: COc1ccc(NC(=O)NC(CO)c2cccc3c2ccn3C)cc1OCCCC(C)C] |
| C5INH- 0469 | 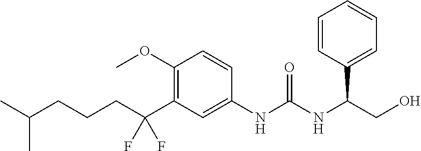 [SMILES: COc1ccc(NC(=O)N[C@H](CO)c2ccccc2)cc1C(F)(F)CCCC(C)C] |
| C5INH- 0472 | 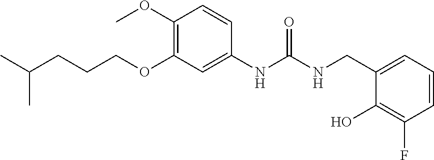 [SMILES: COc1ccc(NC(=O)NCc2cccc(F)c2O)cc1OCCCC(C)C] |
| C5INH- 0473 | 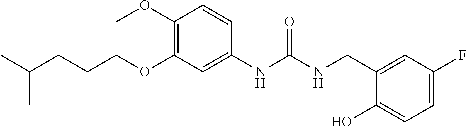 [SMILES: COc1ccc(NC(=O)NCc2cc(F)ccc2O)cc1OCCCC(C)C] |
| C5INH- 0474 | 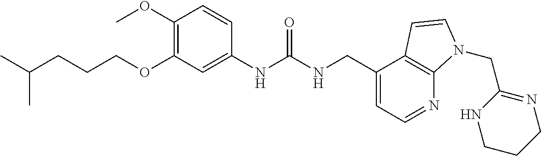 [SMILES: COc1ccc(NC(=O)NCc2ccnc3c2ccn3CC2=NCCCN2)cc1OCCCC(C)C] |
| C5INH- 0476 | 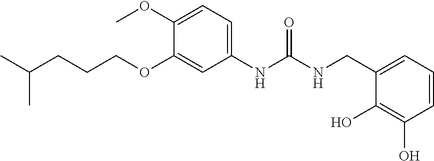 [SMILES: COc1ccc(NC(=O)NCc2cccc(O)c2O)cc1OCCCC(C)C] |
| C5INH- 0477 | 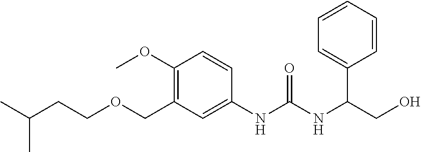 [SMILES: COc1ccc(NC(=O)NC(CO)c2ccccc2)cc1COCCC(C)C] |
| C5INH- 0484 | 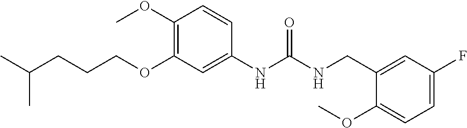 [SMILES: COc1ccc(F)cc1CNC(=O)Nc1ccc(OC)c(OCCCC(C)C)c1] |
| C5INH- 0485 | 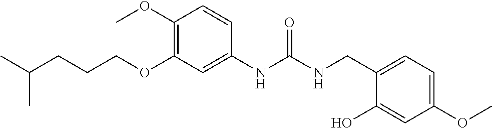 [SMILES: COc1ccc(CNC(=O)Nc2ccc(OC)c(OCCCC(C)C)c2)c(O)c1] |
| C5INH- 0486 | 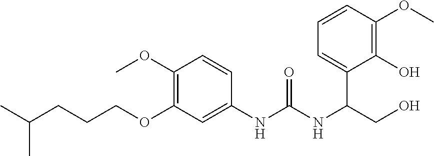 [SMILES: COc1ccc(NC(=O)NC(CO)c2cccc(OC)c2O)cc1OCCCC(C)C] |
| C5INH- 0487 | 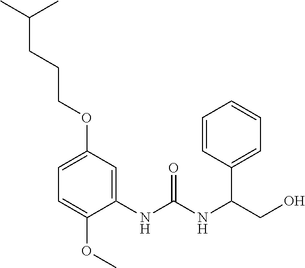 [SMILES: COc1ccc(OCCCC(C)C)cc1NC(=O)NC(CO)c1ccccc1] |
| C5INH- 0488 | 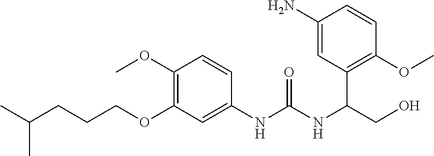 [SMILES: COc1ccc(NC(=O)NC(CO)c2cc(N)ccc2OC)cc1OCCCC(C)C] |
| C5INH- 0489 | 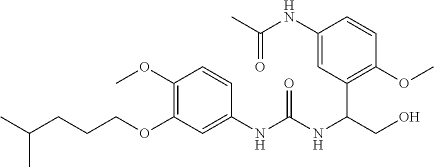 [SMILES: COc1ccc(NC(=O)NC(CO)c2cc(NC(C)=O)ccc2OC)cc1OCCCC(C)C] |
| C5INH- 0490 | 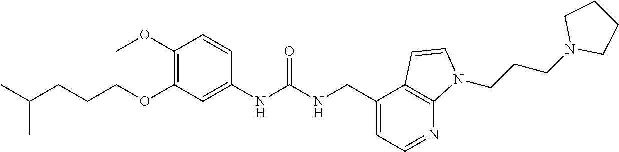 [SMILES: COc1ccc(NC(=O)NCc2ccnc3c2ccn3CCCN2CCCC2)cc1OCCCC(C)C] |
| C5INH- 0491 | 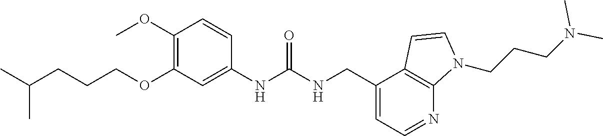 [SMILES: COc1ccc(NC(=O)NCc2ccnc3c2ccn3CCCN(C)C)cc1OCCCC(C)C] |
| C5INH- 0492 | 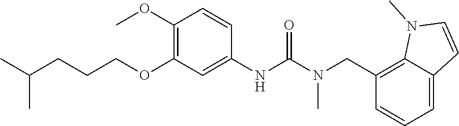 [SMILES: COc1ccc(NC(=O)N(C)Cc2cccc3ccn(C)c23)cc1OCCCC(C)C] |
| C5INH- 0496 | 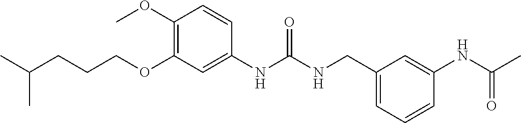 [SMILES: COc1ccc(NC(=O)NCc2cccc(NC(C)=O)c2)cc1OCCCC(C)C] |
| C5INH- 0497 | 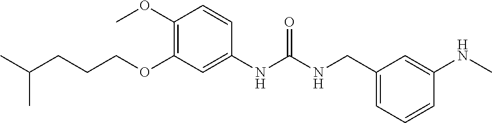 [SMILES: CNc1cccc(CNC(=O)Nc2ccc(OC)c(OCCCC(C)C)c2)c1] |
| C5INH- 0498 | 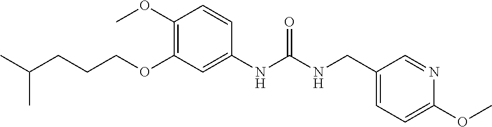 [SMILES: COc1ccc(CNC(=O)Nc2ccc(OC)c(OCCCC(C)C)c2)cn1] |
| C5INH- 0500 | 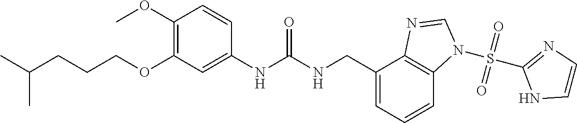 [SMILES: COc1ccc(NC(=O)NCc2cccc3c2ncn3S(=O)(=O)c2ncc[nH]2)cc1OCCCC(C)C] |
| C5INH- 0501 | 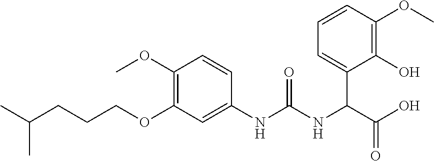 [SMILES: COc1ccc(NC(=O)NC(C(=O)O)c2cccc(OC)c2O)cc1OCCCC(C)C] |
| C5INH- 0502 | 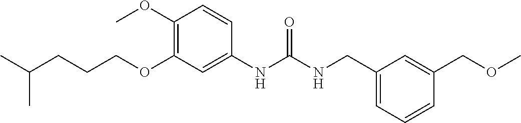 [SMILES: COCc1cccc(CNC(=O)Nc2ccc(OC)c(OCCCC(C)C)c2)c1] |
| C5INH- 0504 | 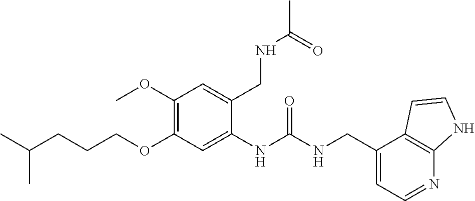 [SMILES: COc1cc(CNC(C)=O)c(NC(=O)NCc2ccnc3[nH]ccc23)cc1OCCCC(C)C] |
| C5INH- 0507 | 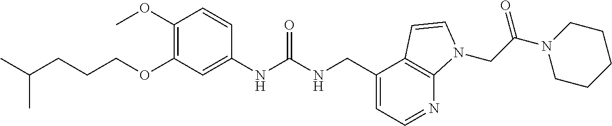 [SMILES: COc1ccc(NC(=O)NCc2ccnc3c2ccn3CC(=O)N2CCCCC2)cc1OCCCC(C)C] |
| C5INH- 0508 | 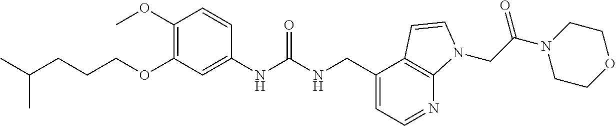 [SMILES: COc1ccc(NC(=O)NCc2ccnc3c2ccn3CC(=O)N2CCOCC2)cc1OCCCC(C)C] |
| C5INH- 0509 | 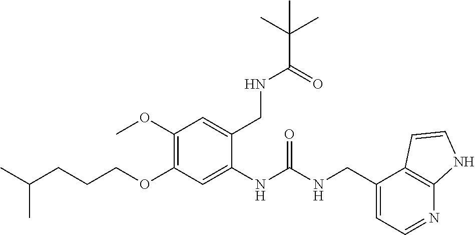 [SMILES: COc1cc(CNC(=O)C(C)(C)C)c(NC(=O)NCc2ccnc3[nH]ccc23)cc1OCCCC(C)C] |
| C5INH- 0510 | 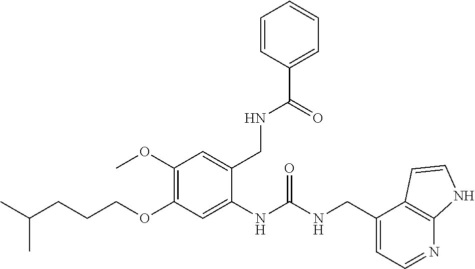 [SMILES: COc1cc(CNC(=O)c2ccccc2)c(NC(=O)NCc2ccnc3[nH]ccc23)cc1OCCCC(C)C] |
| C5INH- 0512 | 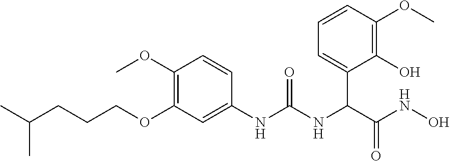 [SMILES: COc1ccc(NC(=O)NC(C(=O)NO)c2cccc(OC)c2O)cc1OCCCC(C)C] |
| C5INH- 0513 | 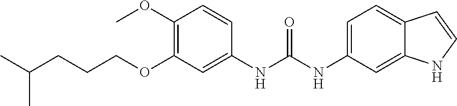 [SMILES: COc1ccc(NC(=O)Nc2ccc3cc[nH]c3c2)cc1OCCCC(C)C] |
| C5INH- 0515 | 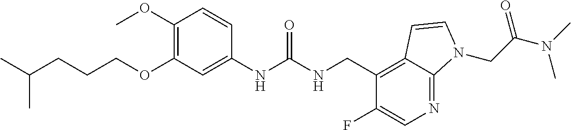 [SMILES: COc1ccc(NC(=O)NCc2c(F)cnc3c2ccn3CC(=O)N(C)C)cc1OCCCC(C)C] |
| C5INH- 0516 | 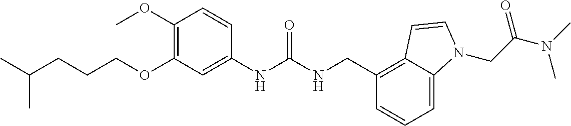 [SMILES: COc1ccc(NC(=O)NCc2cccc3c2ccn3CC(=O)N(C)C)cc1OCCCC(C)C] |
| C5INH- 0517 | 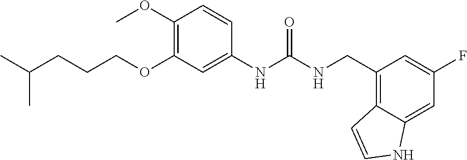 [SMILES: COc1ccc(NC(=O)NCc2cc(F)cc3[nH]ccc23)cc1OCCCC(C)C] |
| C5INH- 0518 | 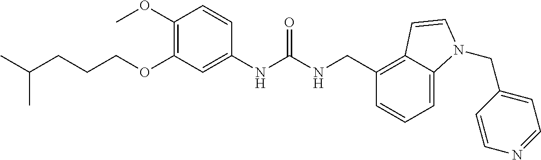 [SMILES: COc1ccc(NC(=O)NCc2cccc3c2ccn3Cc2ccncc2)cc1OCCCC(C)C] |
| C5INH- 0519 | 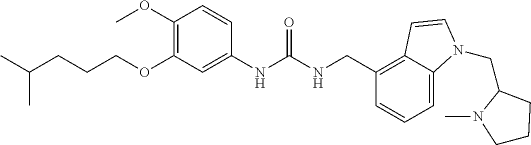 [SMILES: COc1ccc(NC(=O)NCc2cccc3c2ccn3CC2CCCN2C)cc1OCCCC(C)C] |
| C5INH- 0521 | 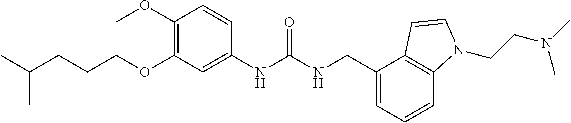 [SMILES: COc1ccc(NC(=O)NCc2cccc3c2ccn3CCN(C)C)cc1OCCCC(C)C] |
| C5INH- 0524 | 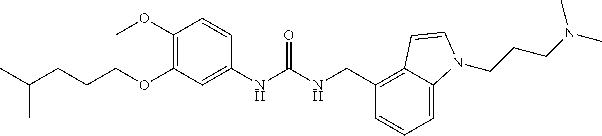 [SMILES: COc1ccc(NC(=O)NCc2cccc3c2ccn3CCCN(C)C)cc1OCCCC(C)C] |
| C5INH- 0525 | 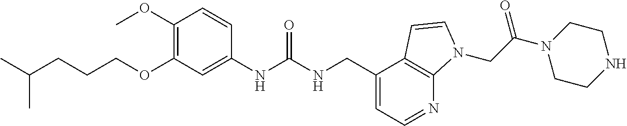 [SMILES: COc1ccc(NC(=O)NCc2ccnc3c2ccn3CC(=O)N2CCNCC2)cc1OCCCC(C)C] |
| C5INH- 0526 | 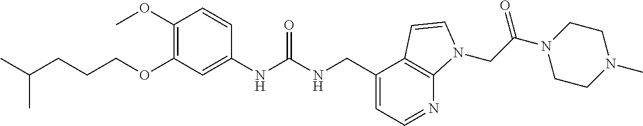 [SMILES: COc1ccc(NC(=O)NCc2ccnc3c2ccn3CC(=O)N2CCN(C)CC2)cc1OCCCC(C)C] |
| C5INH- 0527 | 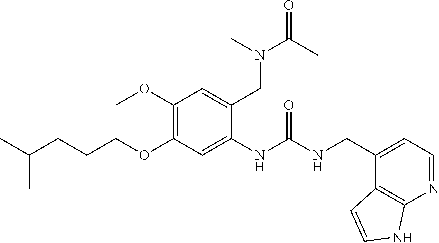 [SMILES: COc1cc(CN(C)C(C)=O)c(NC(=O)NCc2ccnc3[nH]ccc23)cc1OCCCC(C)C] |
| C5INH- 0532 | 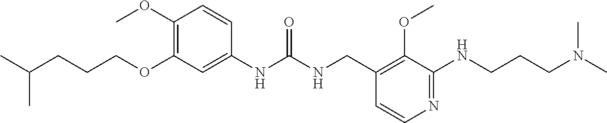 [SMILES: COc1ccc(NC(=O)NCc2ccnc(NCCCN(C)C)c2OC)cc1OCCCC(C)C] |
| C5INH- 0533 | 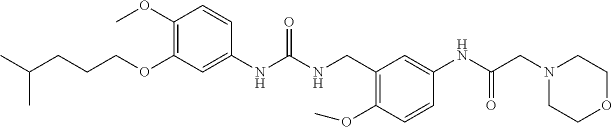 [SMILES: COc1ccc(NC(=O)CN2CCOCC2)cc1CNC(=O)Nc1ccc(OC)c(OCCCC(C)C)c1] |
| C5INH- 0534 | 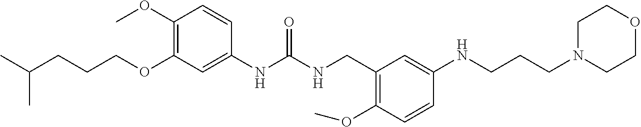 [SMILES: COc1ccc(NCCCN2CCOCC2)cc1CNC(=O)Nc1ccc(OC)c(OCCCC(C)C)c1] |
| C5INH- 0535 | 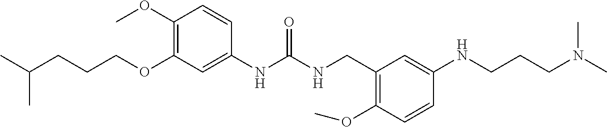 [SMILES: COc1ccc(NCCCN(C)C)cc1CNC(=O)Nc1ccc(OC)c(OCCCC(C)C)c1] |
| C5INH- 0536 | 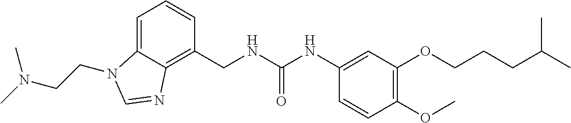 [SMILES: COc1ccc(NC(=O)NCc2cccc3c2ncn3CCN(C)C)cc1OCCCC(C)C] |
| C5INH- 0537 | 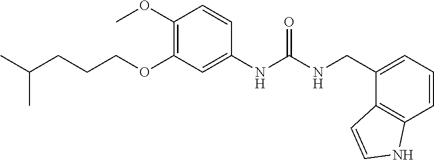 [SMILES: COc1ccc(NC(=O)NCc2cccc3[nH]ccc23)cc1OCCCC(C)C] |
| C5INH- 0538 | 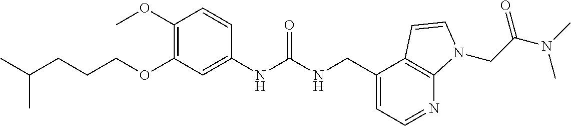 [SMILES: COc1ccc(NC(=O)NCc2ccnc3c2ccn3CC(=O)N(C)C)cc1OCCCC(C)C] |
| C5INH- 0539 | 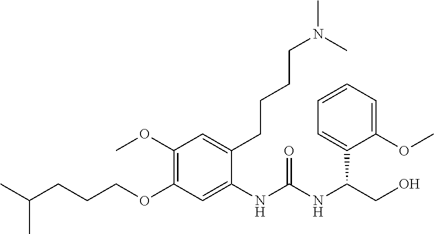 [SMILES: COc1cc(CCCCN(C)C)c(NC(=O)N[C@@H](CO)c2ccccc2OC)cc1OCCCC(C)C] |
| C5INH- 0540 | 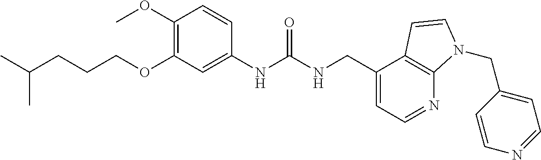 [SMILES: COc1ccc(NC(=O)NCc2ccnc3c2ccn3Cc2ccncc2)cc1OCCCC(C)C] |
| C5INH- 0541 | 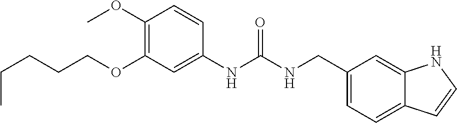 [SMILES: CCCCCOc1cc(NC(=O)NCc2ccc3cc[nH]c3c2)ccc1OC] |
| C5INH- 0543 | 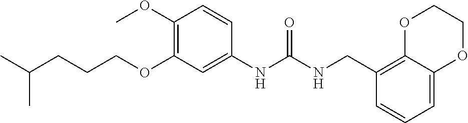 [SMILES: COc1ccc(NC(=O)NCc2cccc3c2OCCO3)cc1OCCCC(C)C] |
| C5INH- 0544 | 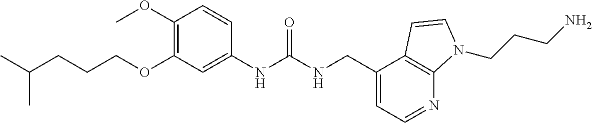 [SMILES: COc1ccc(NC(=O)NCc2ccnc3c2ccn3CCCN)cc1OCCCC(C)C] |
| C5INH- 0545 | 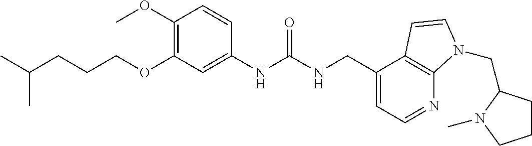 [SMILES: COc1ccc(NC(=O)NCc2ccnc3c2ccn3CC2CCCN2C)cc1OCCCC(C)C] |
| C5INH- 0547 | 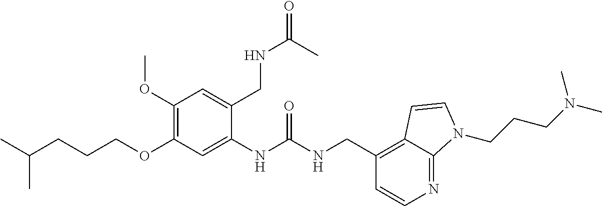 [SMILES: COc1cc(CNC(C)=O)c(NC(=O)NCc2ccnc3c2ccn3CCCN(C)C)cc1OCCCC(C)C] |

### Generic Structure—Formula (Ia)

In some embodiments, a C5 inhibitor compound of the present disclosure has a structure according to Formula (Ia):

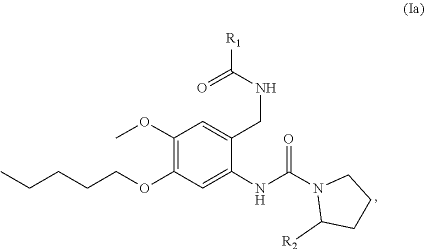

wherein R₁ is any suitable functional group, such as an alkyl, an alkenyl, or an alkynyl, wherein each of the alkyl, alkenyl, or alkynyl is optionally further substituted; and wherein R2 is any suitable functional group, such as a phenyl group, wherein the phenyl group is optionally substituted, such as with one or more halogens.

Non-limiting examples of C5 inhibitor compounds having a structure according to Formula (Ia) include SM0200, SM0201, SM0202, and SM0203.

### Generic Structure—Formula (Ib)

In some embodiments, a C5 inhibitor compound of the present disclosure has a structure according to Formula (Ib):

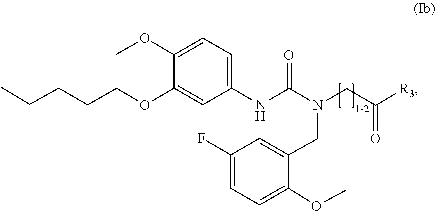

wherein R3 is any suitable functional group, such as —OH or —N(R4)2, and wherein each R4 is independently any suitable functional group, such as hydrogen, an alkyl group, a cyclic group, a heterocyclic group, an aryl group, or a heteroaryl group, wherein each group may be further substituted; or the two R4 groups may form a heterocyclic group, which optionally may be further substituted; wherein the cyclic group or the heterocyclic group may comprise

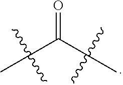 [SMILES: *C(*)=O]

Non-limiting examples of C5 inhibitor compounds having a structure according to Formula (Ib) include SM0204, SM0205, SM0206, SM0207, SM0208, SM0209, SM0210, SM0211, SM0212, SM0213, SM0214, SM0215, and SM0216.

### Generic Structure—Formula (Ic)

In some embodiments, C5 inhibitor compounds have a structure according to Formula (Ic):

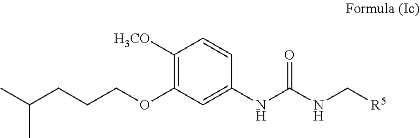

or a pharmaceutically acceptable salt thereof, wherein R⁵ is an optionally substituted cyclic group. The cyclic group may be saturated, aromatic, non-aromatic, unsaturated, or partially unsaturated. The cyclic group may be aryl, heteroaryl, multicyclic, or multi-heterocyclic. The heteroatom of the heteroaryl or multi-heterocyclic group may be O, N, or S. In some embodiments, R⁵ is selected from the group consisting of

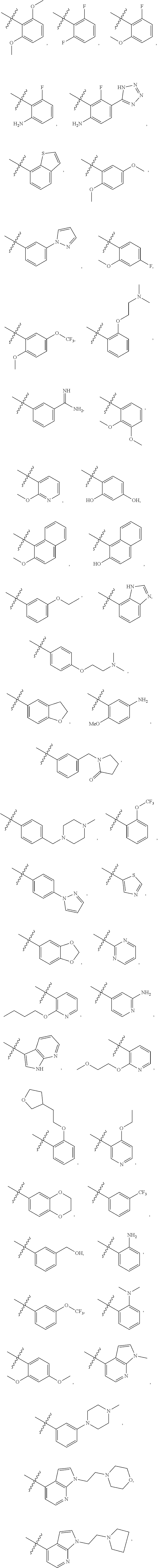

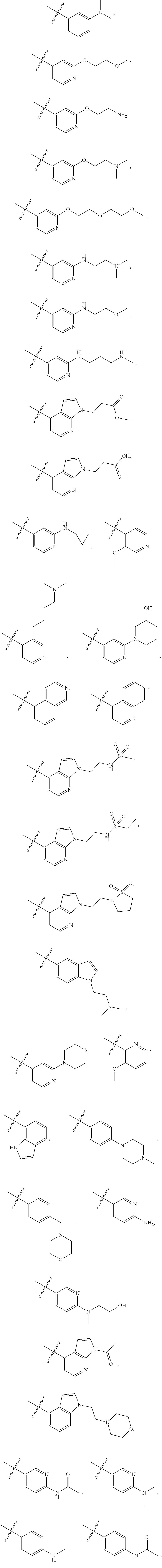

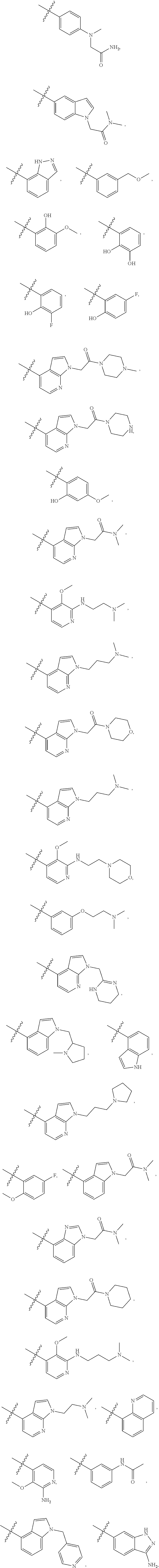

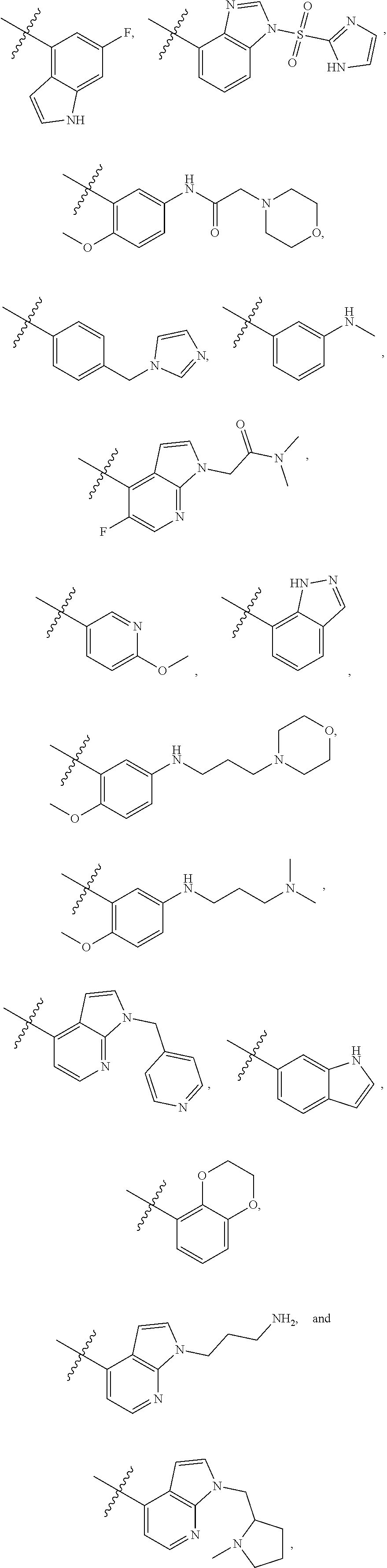

wherein each group may be further substituted.

Non-limiting examples of C5 inhibitor compounds having a structure according to Formula (Ic) include C5INH-0294, C5INH-0296, C5INH-0298, C5INH-0303, C5INH-0310, C5INH-0311, C5INH-0317, C5INH-0318, C5INH-0319, C5INH-0321, C5INH-0323, C5INH-0324, C5INH-0326, C5INH-0329, C5INH-0330, C5INH-0333, C5INH-0335, C5INH-0338, C5INH-0339, C5INH-0340, C5INH-0342, C5INH-0343, C5INH-0348, C5INH-0361, C5INH-0369, C5INH-0370, C5INH-0377, C5INH-0381, C5INH-0382, C5INH-0383, C5INH-0384, C5INH-0389, C5INH-0390, C5INH-0391, C5INH-0398, C5INH-0399, C5INH-0401, C5INH-0402, C5INH-0403, C5INH-0409, C5INH-0410, C5INH-0411, C5INH-0414, C5INH-0417, C5INH-0420, C5INH-0421, C5INH-0428, C5INH-0436, C5INH-0437, C5INH-0443, C5INH-0446, C5INH-0447, C5INH-0448, C5INH-0450, C5INH-0452, C5INH-0453, C5INH-0454, C5INH-0453, C5INH-0456, C5INH-0458, C5INH-0460, C5INH-0462, C5INH-0472, C5INH-0473, C5INH-0474, C5INH-0476, C5INH-0484, C5INH-0485, C5INH-0490, C5INH-0491, C5INH-0496, C5INH-0497, C5INH-0498, C5INH-0500, C5INH-0507, C5INH-0508, C5INH-0516, C5INH-0517, C5INH-0518, C5INH-0521, C5INH-0524, C5INH-0525, C5INH-0526, C5INH-0536, C5INH-0537, C5INH-0538, C5INH-0502, C5INH-0476, C5INH-0534, C5INH-0535, C5INH-0540, C5INH-0541, C5INH-0543, C5INH-0544, and C5INH-0545.

### Generic Structure—Formula (Id)

In some embodiments, C5 inhibitor compounds have a structure according to Formula (Id):

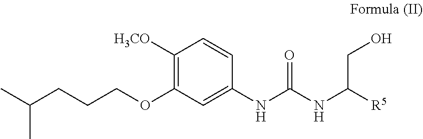

or a pharmaceutically acceptable salt thereof, wherein R⁵ is an optionally substituted cyclic group. The cyclic group may be saturated, aromatic, non-aromatic, unsaturated, or partially unsaturated. The cyclic group may be aryl, heteroaryl, multicyclic, or multi-heterocyclic. The heteroatom of the heteroaryl or multi-heterocyclic group may be O, N, or S. In some embodiments, R⁵ is selected from the group consisting of

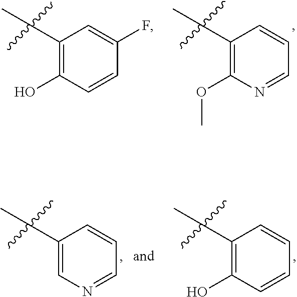

wherein each group may be further substituted.

Non-limiting examples of C5 inhibitor compounds having a structure according to Formula (Id) include C5INH-0315, C5INH-0316, and C5INH-0395.

### Generic Structure—Formula (Ie)

In some embodiments, C5 inhibitor compounds have a structure according to Formula (Ie):

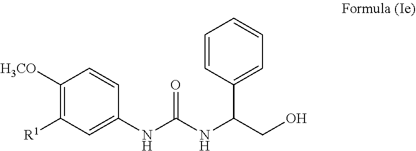

or a pharmaceutically acceptable salt thereof, wherein R¹ is an optionally substituted alkyl group or alkoxyl group. In some embodiments, R1 is selected from —OC₄H₉, —OC₆H₁₃, —OC₇H₁₅, —NH(C₆H₁₃),

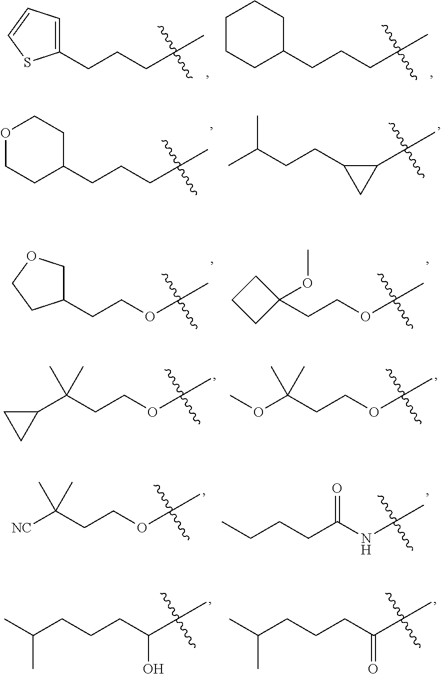

a phenyl group, a toluene group, a pyridine group, a pyrimidine group,

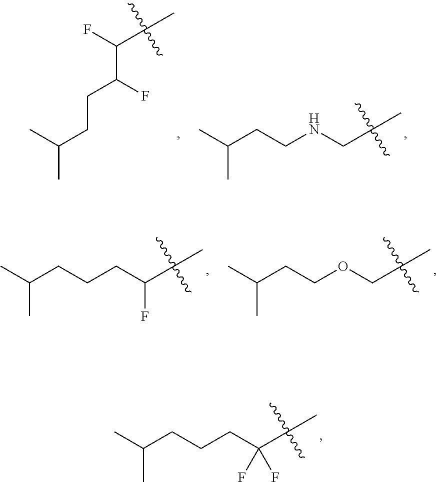

wherein each group may be further substituted.

Non-limiting examples of C5 inhibitor compounds having a structure according to Formula (Ie) include C5INH-0336, C5INH-0349, C5INH-0350, C5INH-0357, C5INH-0367, C5INH-0406, C5INH-0432, C5INH-0477, and C5INH-0469.

### Generic Structure—Formula (If)

In some embodiments, C5 inhibitor compounds have a structure according to Formula (If):

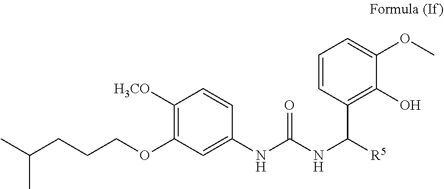

or a pharmaceutically acceptable salt thereof, wherein R⁵ is selected from hydrogen, a C₁-C₈ alkyl group, —CO—NH₂, —CO—NH—OH, aryl, and a heteroaryl group, wherein each group may be substituted with groups such as, but not limited to, a C₁-C₆ aliphatic group, —OH, —O—(C₁-C₄ alkyl), halogen, —CF₃, nitrile, —COOH, —CO—NH₂, —CO—O—NH₂, —OCF₃, —N(H) (C₁-C₄alkyl), and —N—(C₁-C₄ alkyl)₂.

Non-limiting examples of C5 inhibitor compounds having a structure according to Formula (If) include C5INH-0486 and C5INH-0512.

### Generic Structure—Formula (Ig)

In some embodiments, C5 inhibitor compounds have a structure according to Formula (Ig):

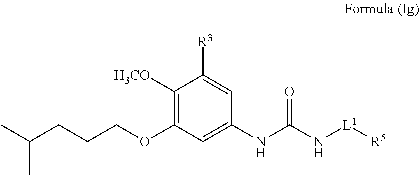

or a pharmaceutically acceptable salt thereof, wherein R³ is not hydrogen. In some embodiments, R3 is selected from

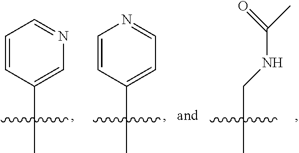

wherein each group may be further substituted;
L¹ may be absent or selected from the group consisting of —C₁-C₆-alkyl-, —C₁-C₆- alkenyl-, -cycloalkyl-, -heterocycle-, -aryl-, and -heteroaryl-, wherein any —CH— may be optionally replaced by —O—, -cycloalkyl-NH—, -alkyl-NH—, —N(R⁶)—, —N(R⁵)—CO—N(R⁷)—, —N(R⁵)—CO—, —CH₂—CO—N(R⁷)—, —N(R⁶)—SO₂—N(R′)—, —SO₂—N(R′)—, —N(R⁷)—SO₂—, —CO—N(R⁷)—, —O—CO—N(R⁷)—, —N(R⁷)—CO—O—, —PO₂—, —P(O)NR⁶—, —O—P(O)NR⁶—O—, —OP(O)O—, —O—PO(R⁶)—O—, —P(OR⁶)₂—, —S(O)NR⁶—, —N(R⁶)—C(═NH)—NR⁷—, and —NR⁶—C(—N(R⁷))═N—, wherein adjacent R⁶ and R⁷ are optionally linked to form a 5-6 membered ring; R⁵ may be selected from hydrogen, R⁶, aryl, and a heteroaryl group; and R⁶ and R⁷ may be independently selected from H and a C1-C8 alkyl group, which may be independently and optionally substituted with groups selected from a C1-C6 aliphatic group; a 3-7 membered saturated, partially saturated, or aromatic ring having zero to three heteroatoms independently selected from nitrogen, sulfur, or oxygen and wherein any of said alkyl group optionally contains 0 to 4 substituents independently selected from —OH, oxo, —O—(C1-C4-alkyl), halogen, —CF₃, nitrile, —COOH, —CO—NH₂, —CO—O—NH₂, —OCF₃, —N(H) (C1-C4-alkyl), and —N—(C1-C4-alkyl)₂.

### Generic Structure—Formula (Ih)

In some embodiments, C5 inhibitor compounds have a structure according to Formula (Ih):

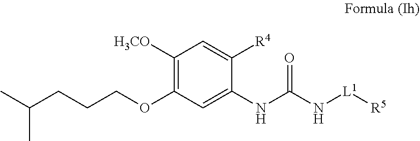

or a pharmaceutically acceptable salt thereof, wherein R⁴ is not hydrogen. In some embodiments, R₄ is selected from —CH₂COOH, —COOMe, —CH₂—CO—NH₂, —CH₂—CO—N(Me)₂, —CH₂—NH₂, —CH₂—NH—CO—CH₃, —CH₂—NH—SO₂—CH₃, —CH₂—NH—CO—C₄H₉, —CH₂—NH—CO—C₆H₅, —CH₂—N(CH₃)—CO—CH₃,

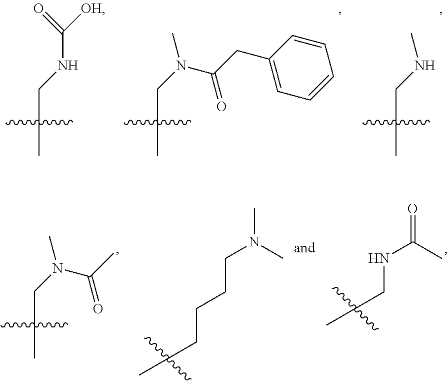

and wherein each group may be further substituted; L¹ may be absent or selected from the group consisting of —C₁-C₆- alkyl-, —C₁-C₆-alkenyl-, -cycloalkyl-, -heterocycle-, -aryl-, and -heteroaryl-, wherein any —CH— may be optionally replaced by —O—, -cycloalkyl-NH—, -alkyl-NH—, —N(R⁶)—, —N(R⁶)—CO—N(R′)—, —N(R⁶)—CO—, —CH₂—CO—N(R⁷)—, —N(R⁶)—SO₂—N(R₇)—, —SO₂—N(R₇)—, —N(R⁷)—SO₂—, —CO—N(R⁷)—, —O—CO—N(R′)—, —N(R⁷)—CO—O—, —PO₂—, —P(O)NR⁶—, —O—P(O)NR⁶—O—, —OP(O)O—, —O—PO(R⁶)—O—P(OR⁶)₂—, —S(O)NR⁶—, —N(R⁶)—C(═NH)—NR⁷—, and —NR⁶—C(—N(R₇))═N—, wherein adjacent R⁶ and R⁷ are optionally linked to form a 5-6 membered ring; R⁵ may be selected from hydrogen, R⁶, aryl, and a heteroaryl group; and R⁶ and R may be independently selected from H and a C1-C8 alkyl group, which may be independently and optionally substituted with groups selected from a C1-C6 aliphatic group; a 3-7 membered saturated, partially saturated, or aromatic ring having zero to three heteroatoms independently selected from nitrogen, sulfur, or oxygen and wherein any of said alkyl group optionally contains 0 to 4 substituents independently selected from —OH, oxo, —O—(C1-C4-alkyl), halogen, —CF₃, nitrile, —COOH, —CO—NH₂, —CO—O—NH₂, —OCF₃, —N(H) (C1-C4-alkyl), and —N—(C1-C4-alkyl)₂.

Non-limiting examples of C5 inhibitor compounds having a structure according to Formula (Ih) include C5INH-0355, C5INH-0397, C5INH-0422, C5INH-0440, C5INH-0504, C5INH-0509, C5INH-0510, C5INH-0527, C5INH-0539, and C5INH-0547.

### 2). Cyclic-Urea C5-Interacting Compounds

In some embodiments, compounds of the present disclosure are C5-interacting compounds. Such compounds may include any of those listed in Table 2, including CU0001-CU0262 and CU0500-CU0847.

TABLE 2
Cyclic-Urea C-interacting compounds

| Compound | |
|---|---|
| Number | Structure |
| CU0001 | 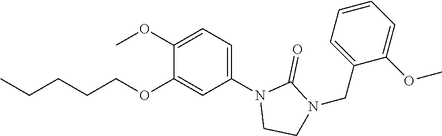 [SMILES: CCCCCOc1cc(N2CCN(Cc3ccccc3OC)C2=O)ccc1OC] |
| CU0002 | 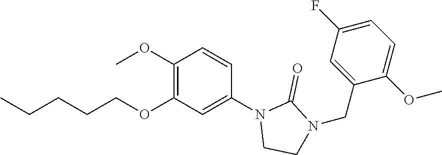 [SMILES: CCCCCOc1cc(N2CCN(Cc3cc(F)ccc3OC)C2=O)ccc1OC] |
| CU0003 | 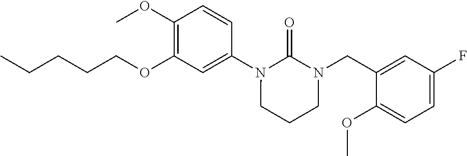 [SMILES: CCCCCOc1cc(N2CCCN(Cc3cc(F)ccc3OC)C2=O)ccc1OC] |
| CU0004 | 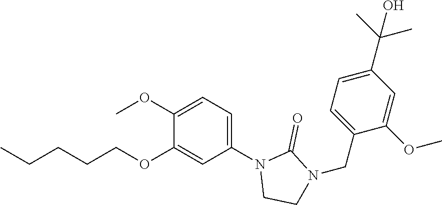 [SMILES: CCCCCOc1cc(N2CCN(Cc3ccc(C(C)(C)O)cc3OC)C2=O)ccc1OC] |
| CU0005 | 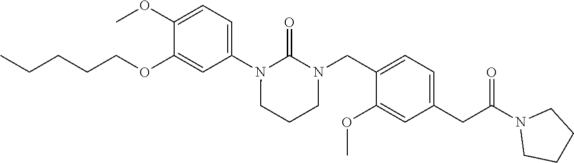 [SMILES: CCCCCOc1cc(N2CCCN(Cc3ccc(CC(=O)N4CCCC4)cc3OC)C2=O)ccc1OC] |
| CU0006 | 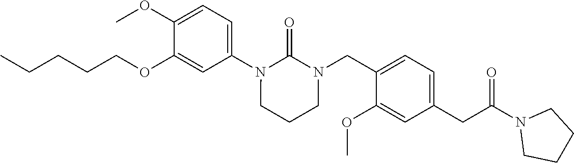 [SMILES: CCCCCOc1cc(N2CCCN(Cc3ccc(CC(=O)N4CCCC4)cc3OC)C2=O)ccc1OC] |
| CU0007 | 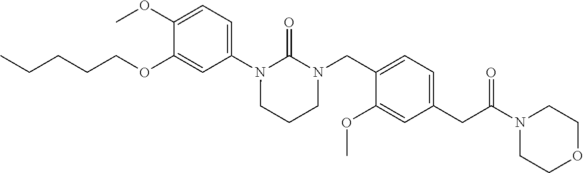 [SMILES: CCCCCOc1cc(N2CCCN(Cc3ccc(CC(=O)N4CCOCC4)cc3OC)C2=O)ccc1OC] |
| CU0008 | 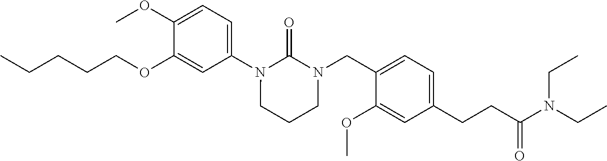 [SMILES: CCCCCOc1cc(N2CCCN(Cc3ccc(CCC(=O)N(CC)CC)cc3OC)C2=O)ccc1OC] |
| CU0009 | 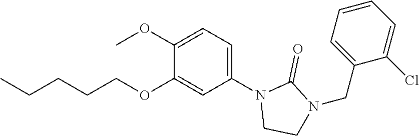 [SMILES: CCCCCOc1cc(N2CCN(Cc3ccccc3Cl)C2=O)ccc1OC] |
| CU0010 | 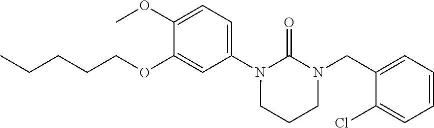 [SMILES: CCCCCOc1cc(N2CCCN(Cc3ccccc3Cl)C2=O)ccc1OC] |
| CU0011 | 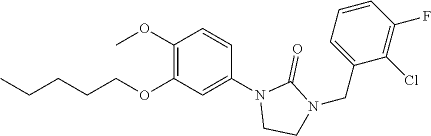 [SMILES: CCCCCOc1cc(N2CCN(Cc3cccc(F)c3Cl)C2=O)ccc1OC] |
| CU0012 | 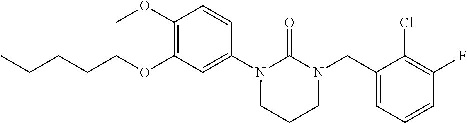 [SMILES: CCCCCOc1cc(N2CCCN(Cc3cccc(F)c3Cl)C2=O)ccc1OC] |
| CU0013 | 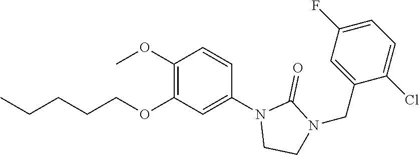 [SMILES: CCCCCOc1cc(N2CCN(Cc3cc(F)ccc3Cl)C2=O)ccc1OC] |
| CU0014 | 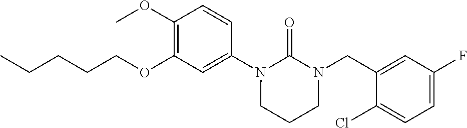 [SMILES: CCCCCOc1cc(N2CCCN(Cc3cc(F)ccc3Cl)C2=O)ccc1OC] |
| CU0015 | 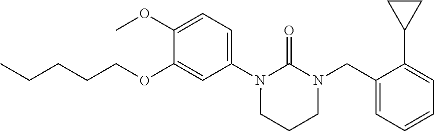 [SMILES: CCCCCOc1cc(N2CCCN(Cc3ccccc3C3CC3)C2=O)ccc1OC] |
| CU0016 | 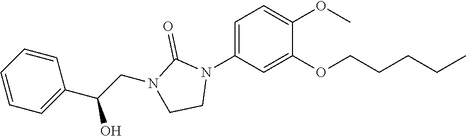 [SMILES: CCCCCOc1cc(N2CCN(C[C@@H](O)c3ccccc3)C2=O)ccc1OC] |
| CU0017 | 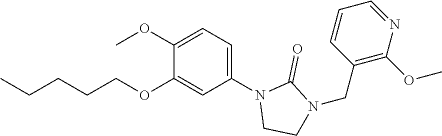 [SMILES: CCCCCOc1cc(N2CCN(Cc3cccnc3OC)C2=O)ccc1OC] |
| CU0018 | 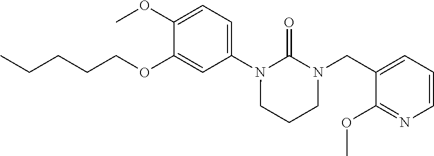 [SMILES: CCCCCOc1cc(N2CCCN(Cc3cccnc3OC)C2=O)ccc1OC] |
| CU0019 | 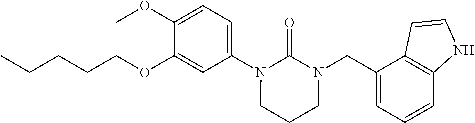 [SMILES: CCCCCOc1cc(N2CCCN(Cc3cccc4[nH]ccc34)C2=O)ccc1OC] |
| CU0020 | 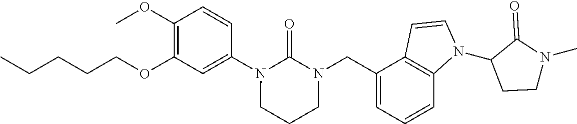 [SMILES: CCCCCOc1cc(N2CCCN(Cc3cccc4c3ccn4C3CCN(C)C3=O)C2=O)ccc1OC] |
| CU0021 | 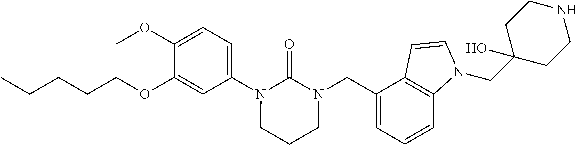 [SMILES: CCCCCOc1cc(N2CCCN(Cc3cccc4c3ccn4CC3(O)CCNCC3)C2=O)ccc1OC] |
| CU0022 | 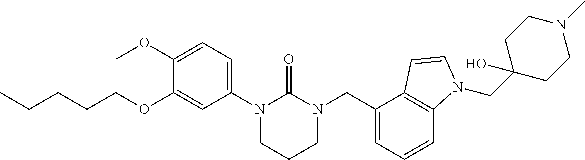 [SMILES: CCCCCOc1cc(N2CCCN(Cc3cccc4c3ccn4CC3(O)CCN(C)CC3)C2=O)ccc1OC] |
| CU0023 | 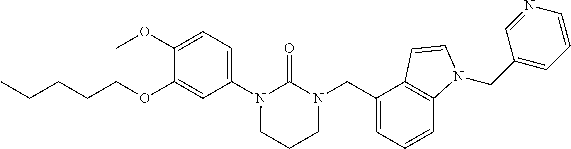 [SMILES: CCCCCOc1cc(N2CCCN(Cc3cccc4c3ccn4Cc3cccnc3)C2=O)ccc1OC] |
| CU0024 | 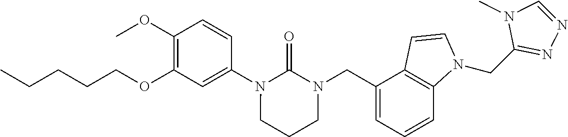 [SMILES: CCCCCOc1cc(N2CCCN(Cc3cccc4c3ccn4Cc3nncn3C)C2=O)ccc1OC] |
| CU0025 | 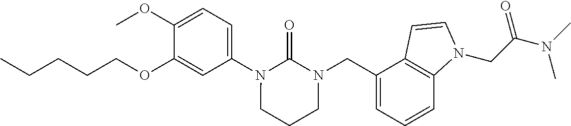 [SMILES: CCCCCOc1cc(N2CCCN(Cc3cccc4c3ccn4CC(=O)N(C)C)C2=O)ccc1OC] |
| CU0026 | 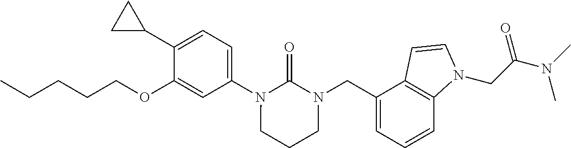 [SMILES: CCCCCOc1cc(N2CCCN(Cc3cccc4c3ccn4CC(=O)N(C)C)C2=O)ccc1C1CC1] |
| CU0027 | 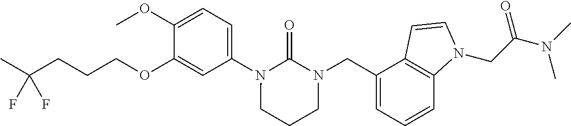 [SMILES: COc1ccc(N2CCCN(Cc3cccc4c3ccn4CC(=O)N(C)C)C2=O)cc1OCCCC(C)(F)F] |
| CU0028 | 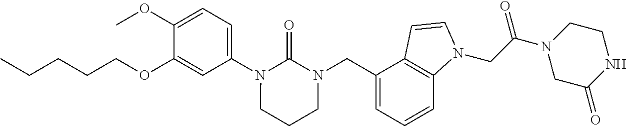 [SMILES: CCCCCOc1cc(N2CCCN(Cc3cccc4c3ccn4CC(=O)N3CCNC(=O)C3)C2=O)ccc1OC] |
| CU0029 | 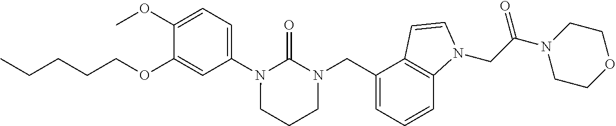 [SMILES: CCCCCOc1cc(N2CCCN(Cc3cccc4c3ccn4CC(=O)N3CCOCC3)C2=O)ccc1OC] |
| CU0030 | 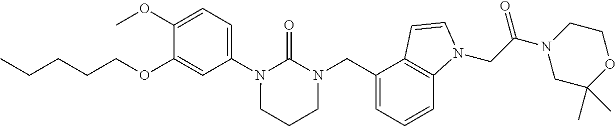 [SMILES: CCCCCOc1cc(N2CCCN(Cc3cccc4c3ccn4CC(=O)N3CCOC(C)(C)C3)C2=O)ccc1OC] |
| CU0031 | 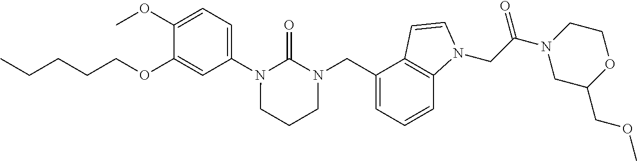 [SMILES: CCCCCOc1cc(N2CCCN(Cc3cccc4c3ccn4CC(=O)N3CCOC(COC)C3)C2=O)ccc1OC] |
| CU0032 | 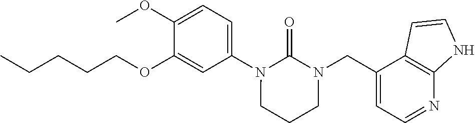 [SMILES: CCCCCOc1cc(N2CCCN(Cc3ccnc4[nH]ccc34)C2=O)ccc1OC] |
| CU0033 | 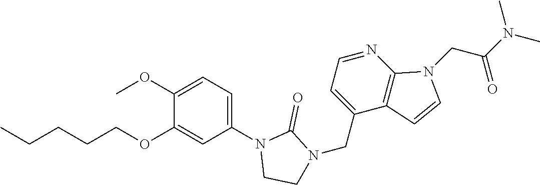 [SMILES: CCCCCOc1cc(N2CCN(Cc3ccnc4c3ccn4CC(=O)N(C)C)C2=O)ccc1OC] |
| CU0034 | 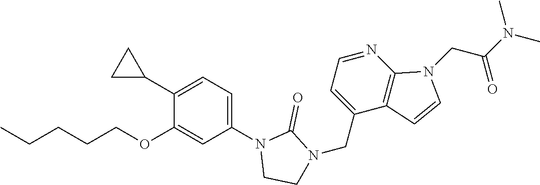 [SMILES: CCCCCOc1cc(N2CCN(Cc3ccnc4c3ccn4CC(=O)N(C)C)C2=O)ccc1C1CC1] |
| CU0035 | 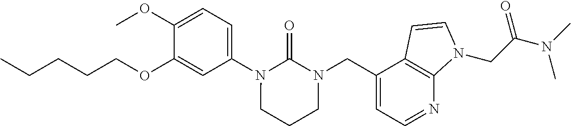 [SMILES: CCCCCOc1cc(N2CCCN(Cc3ccnc4c3ccn4CC(=O)N(C)C)C2=O)ccc1OC] |
| CU0036 | 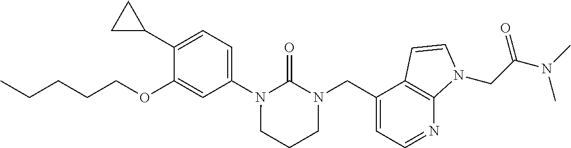 [SMILES: CCCCCOc1cc(N2CCCN(Cc3ccnc4c3ccn4CC(=O)N(C)C)C2=O)ccc1C1CC1] |
| CU0037 | 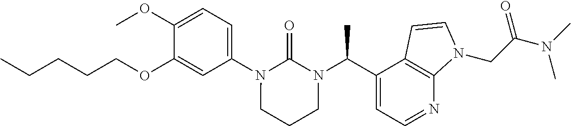 [SMILES: CCCCCOc1cc(N2CCCN([C@@H](C)c3ccnc4c3ccn4CC(=O)N(C)C)C2=O)ccc1OC] |
| CU0038 | 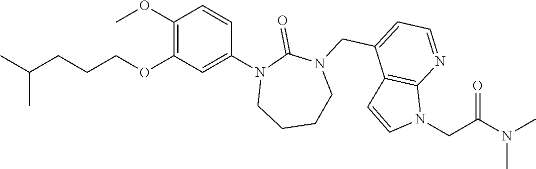 [SMILES: COc1ccc(N2CCCCN(Cc3ccnc4c3ccn4CC(=O)N(C)C)C2=O)cc1OCCCC(C)C] |
| CU0039 | 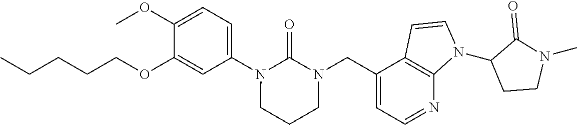 [SMILES: CCCCCOc1cc(N2CCCN(Cc3ccnc4c3ccn4C3CCN(C)C3=O)C2=O)ccc1OC] |
| CU0040 | 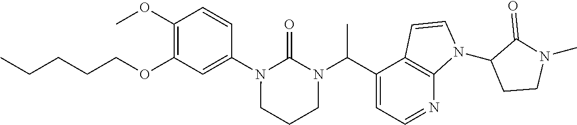 [SMILES: CCCCCOc1cc(N2CCCN(C(C)c3ccnc4c3ccn4C3CCN(C)C3=O)C2=O)ccc1OC] |
| CU0041 | 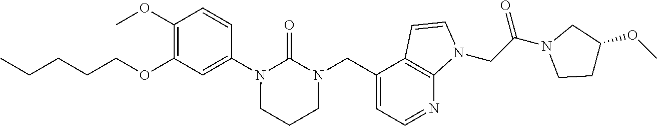 [SMILES: CCCCCOc1cc(N2CCCN(Cc3ccnc4c3ccn4CC(=O)N3CC[C@@H](OC)C3)C2=O)ccc1OC] |
| CU0042 | 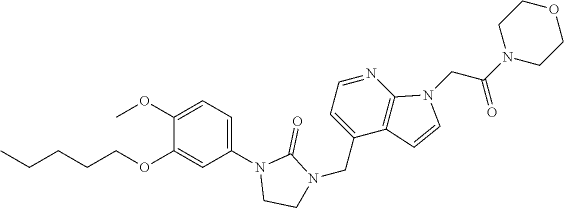 [SMILES: CCCCCOc1cc(N2CCN(Cc3ccnc4c3ccn4CC(=O)N3CCOCC3)C2=O)ccc1OC] |
| CU0043 | 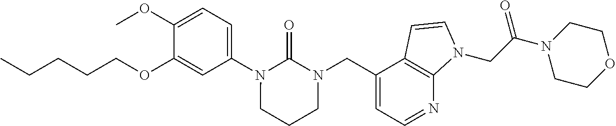 [SMILES: CCCCCOc1cc(N2CCCN(Cc3ccnc4c3ccn4CC(=O)N3CCOCC3)C2=O)ccc1OC] |
| CU0044 | 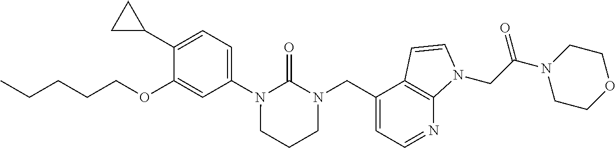 [SMILES: CCCCCOc1cc(N2CCCN(Cc3ccnc4c3ccn4CC(=O)N3CCOCC3)C2=O)ccc1C1CC1] |
| CU0045 | 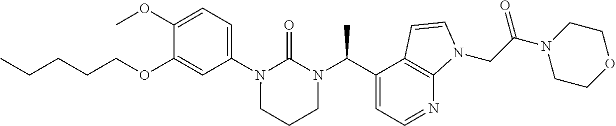 [SMILES: CCCCCOc1cc(N2CCCN([C@@H](C)c3ccnc4c3ccn4CC(=O)N3CCOCC3)C2=O)ccc1OC] |
| CU0046 | 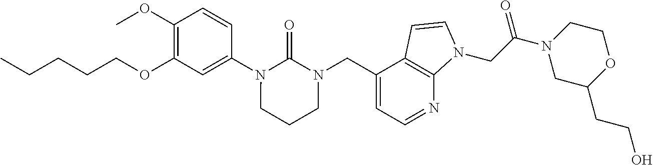 [SMILES: CCCCCOc1cc(N2CCCN(Cc3ccnc4c3ccn4CC(=O)N3CCOC(CCO)C3)C2=O)ccc1OC] |
| CU0047 | 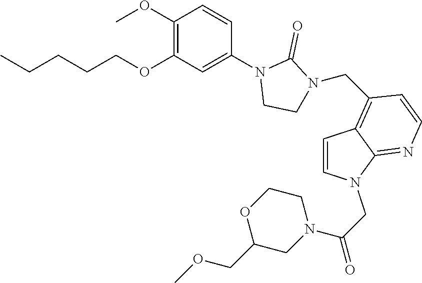 [SMILES: CCCCCOc1cc(N2CCN(Cc3ccnc4c3ccn4CC(=O)N3CCOC(COC)C3)C2=O)ccc1OC] |
| CU0048 | 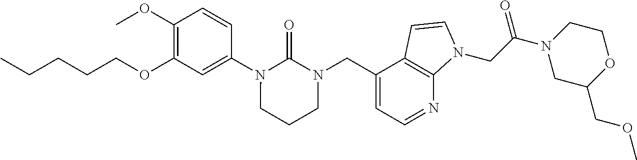 [SMILES: CCCCCOc1cc(N2CCCN(Cc3ccnc4c3ccn4CC(=O)N3CCOC(COC)C3)C2=O)ccc1OC] |
| CU0049 | 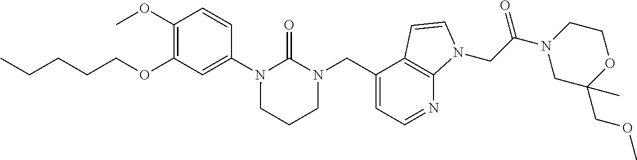 [SMILES: CCCCCOc1cc(N2CCCN(Cc3ccnc4c3ccn4CC(=O)N3CCOC(C)(COC)C3)C2=O)ccc1OC] |
| CU0050 | 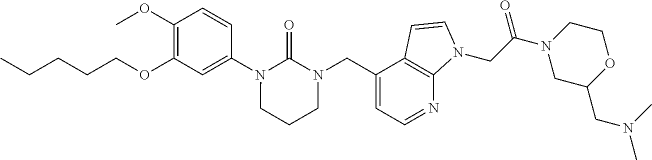 [SMILES: CCCCCOc1cc(N2CCCN(Cc3ccnc4c3ccn4CC(=O)N3CCOC(CN(C)C)C3)C2=O)ccc1OC] |
| CU0051 | 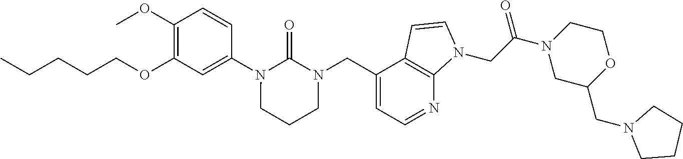 [SMILES: CCCCCOc1cc(N2CCCN(Cc3ccnc4c3ccn4CC(=O)N3CCOC(CN4CCCC4)C3)C2=O)ccc1OC] |
| CU0052 | 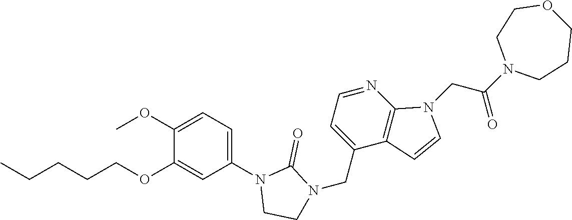 [SMILES: CCCCCOc1cc(N2CCN(Cc3ccnc4c3ccn4CC(=O)N3CCCOCC3)C2=O)ccc1OC] |
| CU0053 | 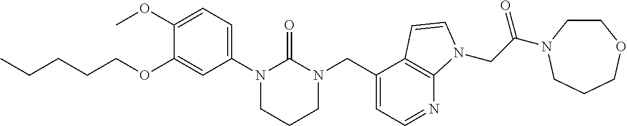 [SMILES: CCCCCOc1cc(N2CCCN(Cc3ccnc4c3ccn4CC(=O)N3CCCOCC3)C2=O)ccc1OC] |
| CU0054 | 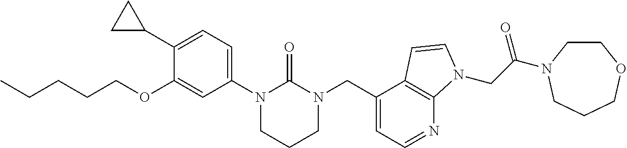 [SMILES: CCCCCOc1cc(N2CCCN(Cc3ccnc4c3ccn4CC(=O)N3CCCOCC3)C2=O)ccc1C1CC1] |
| CU0055 | 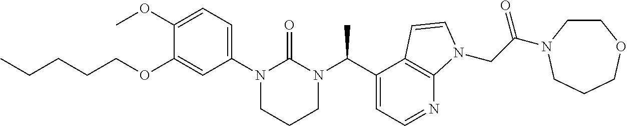 [SMILES: CCCCCOc1cc(N2CCCN([C@@H](C)c3ccnc4c3ccn4CC(=O)N3CCCOCC3)C2=O)ccc1OC] |
| CU0056 | 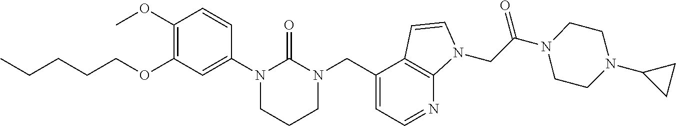 [SMILES: CCCCCOc1cc(N2CCCN(Cc3ccnc4c3ccn4CC(=O)N3CCN(C4CC4)CC3)C2=O)ccc1OC] |
| CU0057 | 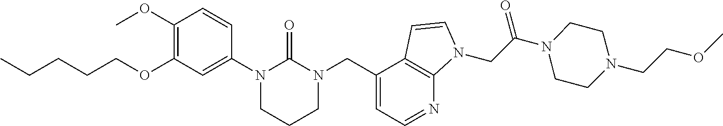 [SMILES: CCCCCOc1cc(N2CCCN(Cc3ccnc4c3ccn4CC(=O)N3CCN(CCOC)CC3)C2=O)ccc1OC] |
| CU0058 | 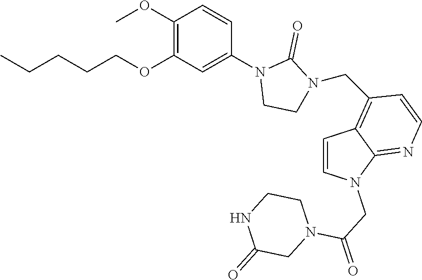 [SMILES: CCCCCOc1cc(N2CCN(Cc3ccnc4c3ccn4CC(=O)N3CCNC(=O)C3)C2=O)ccc1OC] |
| CU0059 | 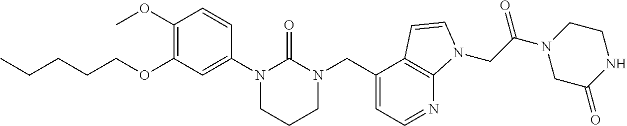 [SMILES: CCCCCOc1cc(N2CCCN(Cc3ccnc4c3ccn4CC(=O)N3CCNC(=O)C3)C2=O)ccc1OC] |
| CU0060 | 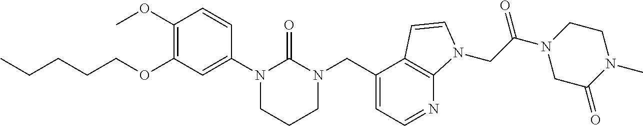 [SMILES: CCCCCOc1cc(N2CCCN(Cc3ccnc4c3ccn4CC(=O)N3CCN(C)C(=O)C3)C2=O)ccc1OC] |
| CU0061 | 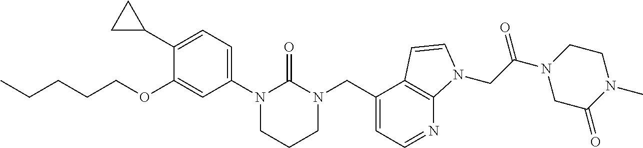 [SMILES: CCCCCOc1cc(N2CCCN(Cc3ccnc4c3ccn4CC(=O)N3CCN(C)C(=O)C3)C2=O)ccc1C1CC1] |
| CU0062 | 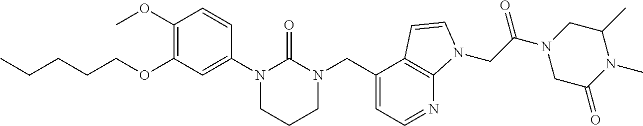 [SMILES: CCCCCOc1cc(N2CCCN(Cc3ccnc4c3ccn4CC(=O)N3CC(=O)N(C)C(C)C3)C2=O)ccc1OC] |
| CU0063 | 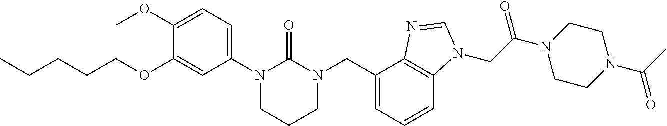 [SMILES: CCCCCOc1cc(N2CCCN(Cc3cccc4c3ncn4CC(=O)N3CCN(C(C)=O)CC3)C2=O)ccc1OC] |
| CU0064 | 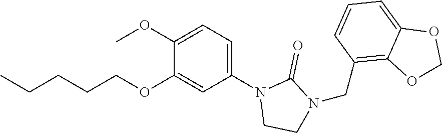 [SMILES: CCCCCOc1cc(N2CCN(Cc3cccc4c3OCO4)C2=O)ccc1OC] |
| CU0065 | 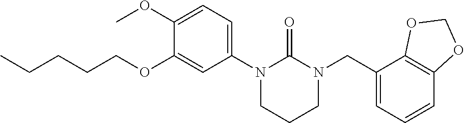 [SMILES: CCCCCOc1cc(N2CCCN(Cc3cccc4c3OCO4)C2=O)ccc1OC] |
| CU0066 | 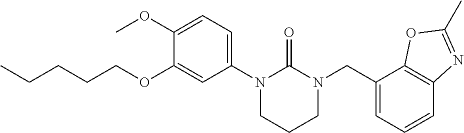 [SMILES: CCCCCOc1cc(N2CCCN(Cc3cccc4nc(C)oc34)C2=O)ccc1OC] |
| CU0067 | 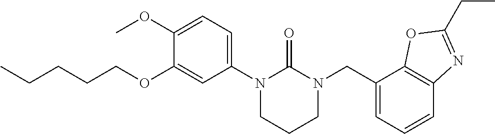 [SMILES: CCCCCOc1cc(N2CCCN(Cc3cccc4nc(CC)oc34)C2=O)ccc1OC] |
| CU0100 | 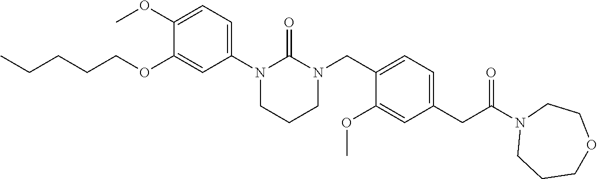 [SMILES: CCCCCOc1cc(N2CCCN(Cc3ccc(CC(=O)N4CCCOCC4)cc3OC)C2=O)ccc1OC] |
| CU0101 | 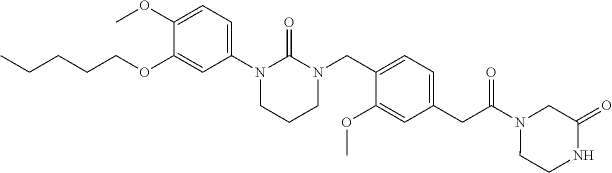 [SMILES: CCCCCOc1cc(N2CCCN(Cc3ccc(CC(=O)N4CCNC(=O)C4)cc3OC)C2=O)ccc1OC] |
| CU0102 | 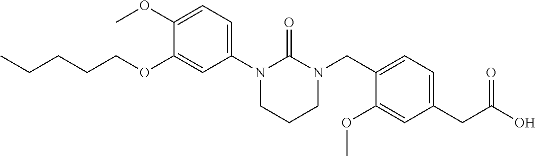 [SMILES: CCCCCOc1cc(N2CCCN(Cc3ccc(CC(=O)O)cc3OC)C2=O)ccc1OC] |
| CU0103 | 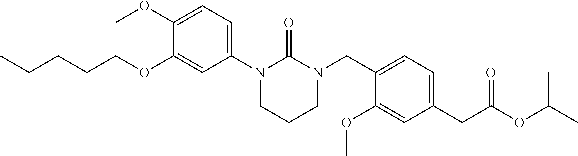 [SMILES: CCCCCOc1cc(N2CCCN(Cc3ccc(CC(=O)OC(C)C)cc3OC)C2=O)ccc1OC] |
| CU0104 | 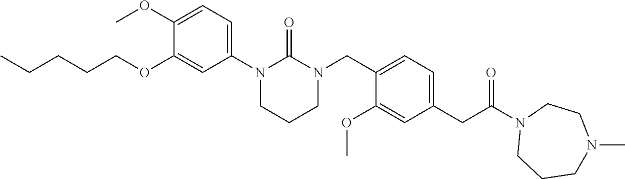 [SMILES: CCCCCOc1cc(N2CCCN(Cc3ccc(CC(=O)N4CCCN(C)CC4)cc3OC)C2=O)ccc1OC] |
| CU0105 | 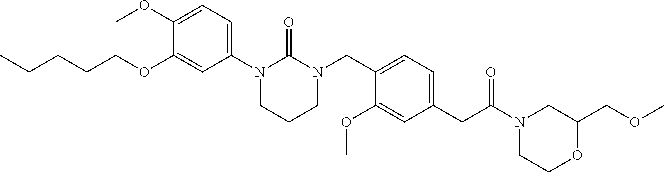 [SMILES: CCCCCOc1cc(N2CCCN(Cc3ccc(CC(=O)N4CCOC(COC)C4)cc3OC)C2=O)ccc1OC] |
| CU0106 | 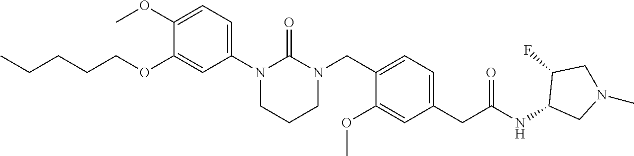 [SMILES: CCCCCOc1cc(N2CCCN(Cc3ccc(CC(=O)N[C@H]4CN(C)C[C@H]4F)cc3OC)C2=O)ccc1OC] |
| CU0107 | 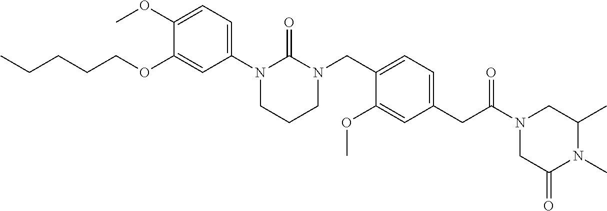 [SMILES: CCCCCOc1cc(N2CCCN(Cc3ccc(CC(=O)N4CC(=O)N(C)C(C)C4)cc3OC)C2=O)ccc1OC] |
| CU0108 | 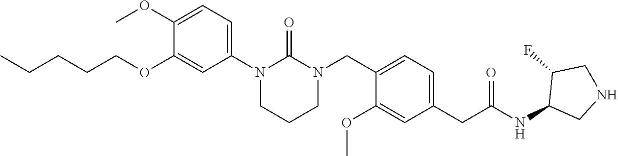 [SMILES: CCCCCOc1cc(N2CCCN(Cc3ccc(CC(=O)N[C@@H]4CNC[C@H]4F)cc3OC)C2=O)ccc1OC] |
| CU0109 | 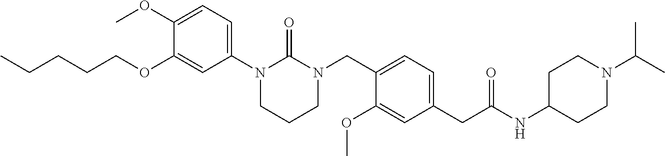 [SMILES: CCCCCOc1cc(N2CCCN(Cc3ccc(CC(=O)NC4CCN(C(C)C)CC4)cc3OC)C2=O)ccc1OC] |
| CU0110 | 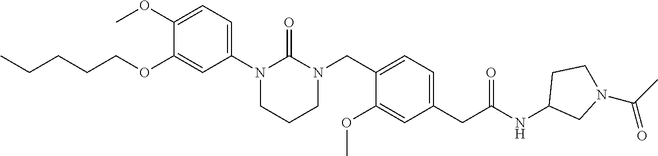 [SMILES: CCCCCOc1cc(N2CCCN(Cc3ccc(CC(=O)NC4CCN(C(C)=O)C4)cc3OC)C2=O)ccc1OC] |
| CU0111 | 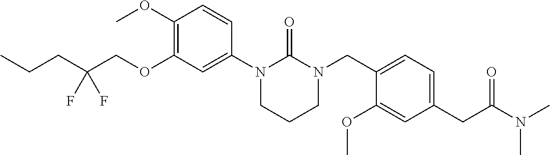 [SMILES: CCCC(F)(F)COc1cc(N2CCCN(Cc3ccc(CC(=O)N(C)C)cc3OC)C2=O)ccc1OC] |
| CU0112 | 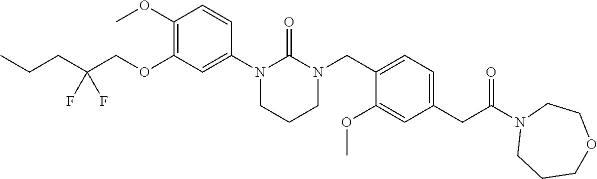 [SMILES: CCCC(F)(F)COc1cc(N2CCCN(Cc3ccc(CC(=O)N4CCCOCC4)cc3OC)C2=O)ccc1OC] |
| CU0113 | 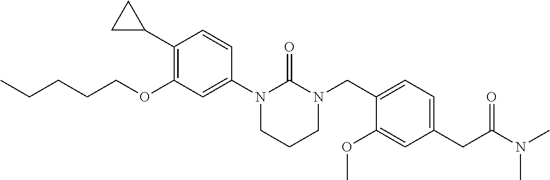 [SMILES: CCCCCOc1cc(N2CCCN(Cc3ccc(CC(=O)N(C)C)cc3OC)C2=O)ccc1C1CC1] |
| CU0114 | 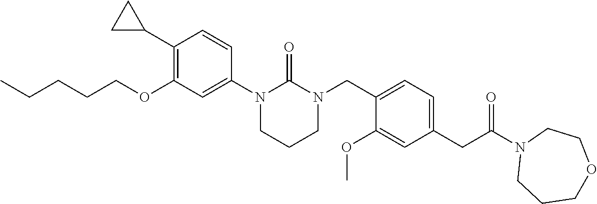 [SMILES: CCCCCOc1cc(N2CCCN(Cc3ccc(CC(=O)N4CCCOCC4)cc3OC)C2=O)ccc1C1CC1] |
| CU0115 | 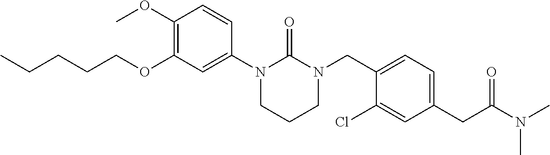 [SMILES: CCCCCOc1cc(N2CCCN(Cc3ccc(CC(=O)N(C)C)cc3Cl)C2=O)ccc1OC] |
| CU0116 | 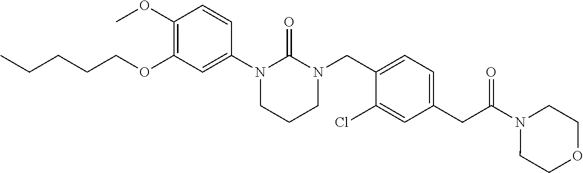 [SMILES: CCCCCOc1cc(N2CCCN(Cc3ccc(CC(=O)N4CCOCC4)cc3Cl)C2=O)ccc1OC] |
| CU0117 | 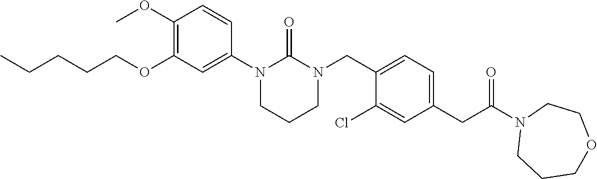 [SMILES: CCCCCOc1cc(N2CCCN(Cc3ccc(CC(=O)N4CCCOCC4)cc3Cl)C2=O)ccc1OC] |
| CU0118 | 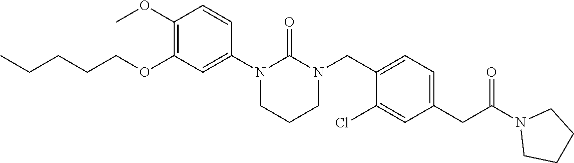 [SMILES: CCCCCOc1cc(N2CCCN(Cc3ccc(CC(=O)N4CCCC4)cc3Cl)C2=O)ccc1OC] |
| CU0119 | 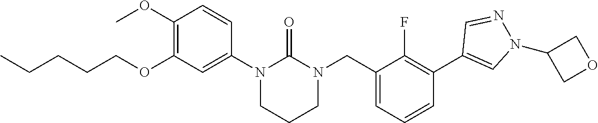 [SMILES: CCCCCOc1cc(N2CCCN(Cc3cccc(-c4cnn(C5COC5)c4)c3F)C2=O)ccc1OC] |
| CU0120 | 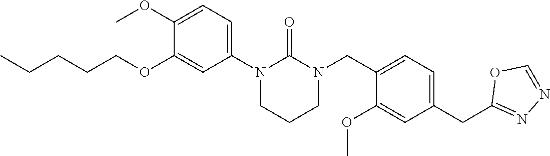 [SMILES: CCCCCOc1cc(N2CCCN(Cc3ccc(Cc4nnco4)cc3OC)C2=O)ccc1OC] |
| CU0121 | 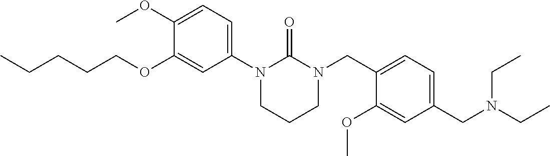 [SMILES: CCCCCOc1cc(N2CCCN(Cc3ccc(CN(CC)CC)cc3OC)C2=O)ccc1OC] |
| CU0122 | 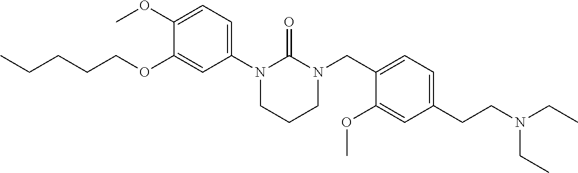 [SMILES: CCCCCOc1cc(N2CCCN(Cc3ccc(CCN(CC)CC)cc3OC)C2=O)ccc1OC] |
| CU0123 | 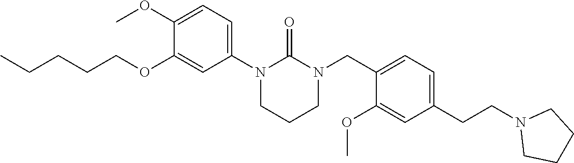 [SMILES: CCCCCOc1cc(N2CCCN(Cc3ccc(CCN4CCCC4)cc3OC)C2=O)ccc1OC] |
| CU0124 | 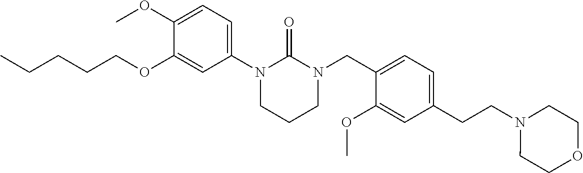 [SMILES: CCCCCOc1cc(N2CCCN(Cc3ccc(CCN4CCOCC4)cc3OC)C2=O)ccc1OC] |
| CU0125 | 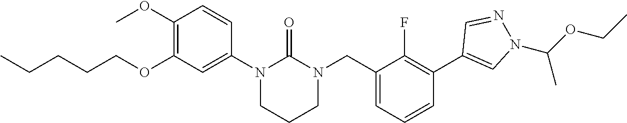 [SMILES: CCCCCOc1cc(N2CCCN(Cc3cccc(-c4cnn(C(C)OCC)c4)c3F)C2=O)ccc1OC] |
| CU0126 | 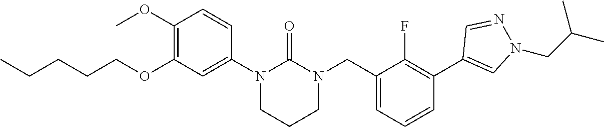 [SMILES: CCCCCOc1cc(N2CCCN(Cc3cccc(-c4cnn(CC(C)C)c4)c3F)C2=O)ccc1OC] |
| CU0127 | 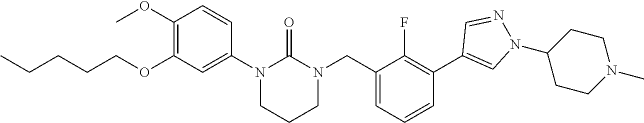 [SMILES: CCCCCOc1cc(N2CCCN(Cc3cccc(-c4cnn(C5CCN(C)CC5)c4)c3F)C2=O)ccc1OC] |
| CU0128 | 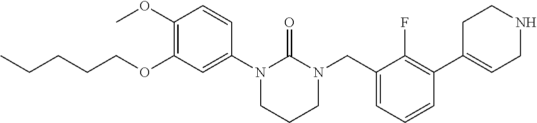 [SMILES: CCCCCOc1cc(N2CCCN(Cc3cccc(C4=CCNCC4)c3F)C2=O)ccc1OC] |
| CU0129 | 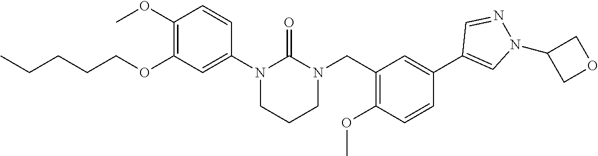 [SMILES: CCCCCOc1cc(N2CCCN(Cc3cc(-c4cnn(C5COC5)c4)ccc3OC)C2=O)ccc1OC] |
| CU0130 | 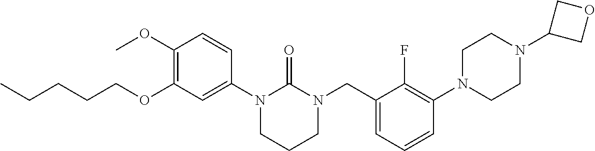 [SMILES: CCCCCOc1cc(N2CCCN(Cc3cccc(N4CCN(C5COC5)CC4)c3F)C2=O)ccc1OC] |
| CU0131 | 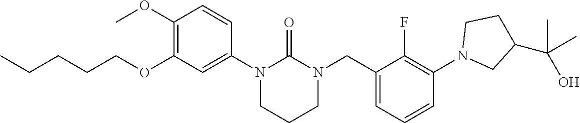 [SMILES: CCCCCOc1cc(N2CCCN(Cc3cccc(N4CCC(C(C)(C)O)C4)c3F)C2=O)ccc1OC] |
| CU0132 | 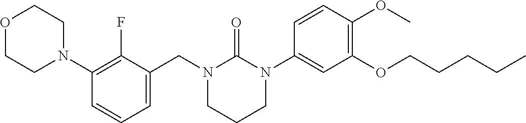 [SMILES: CCCCCOc1cc(N2CCCN(Cc3cccc(N4CCOCC4)c3F)C2=O)ccc1OC] |
| CU0133 | 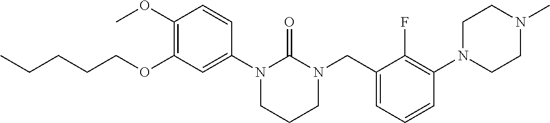 [SMILES: CCCCCOc1cc(N2CCCN(Cc3cccc(N4CCN(C)CC4)c3F)C2=O)ccc1OC] |
| CU0134 | 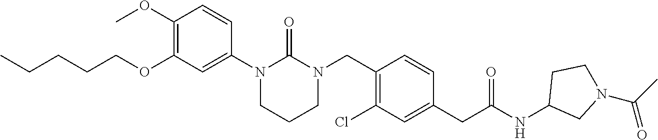 [SMILES: CCCCCOc1cc(N2CCCN(Cc3ccc(CC(=O)NC4CCN(C(C)=O)C4)cc3Cl)C2=O)ccc1OC] |
| CU0135 | 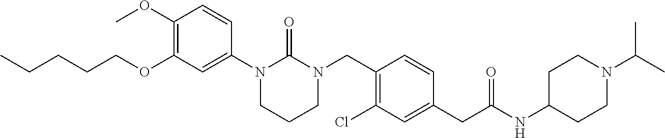 [SMILES: CCCCCOc1cc(N2CCCN(Cc3ccc(CC(=O)NC4CCN(C(C)C)CC4)cc3Cl)C2=O)ccc1OC] |
| CU0136 | 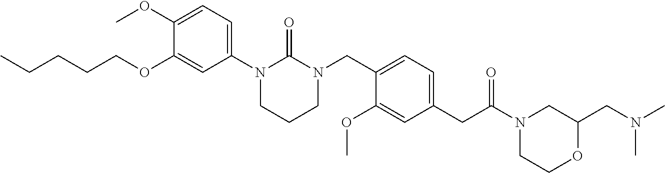 [SMILES: CCCCCOc1cc(N2CCCN(Cc3ccc(CC(=O)N4CCOC(CN(C)C)C4)cc3OC)C2=O)ccc1OC] |
| CU0137 | 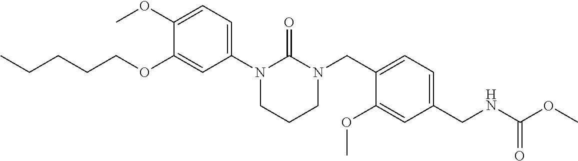 [SMILES: CCCCCOc1cc(N2CCCN(Cc3ccc(CNC(=O)OC)cc3OC)C2=O)ccc1OC] |
| CU0138 | 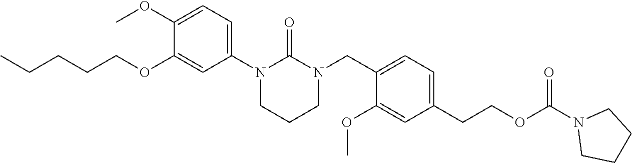 [SMILES: CCCCCOc1cc(N2CCCN(Cc3ccc(CCOC(=O)N4CCCC4)cc3OC)C2=O)ccc1OC] |
| CU0139 | 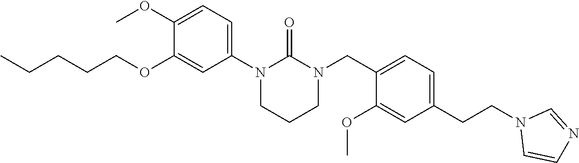 [SMILES: CCCCCOc1cc(N2CCCN(Cc3ccc(CCn4ccnc4)cc3OC)C2=O)ccc1OC] |
| CU0140 | 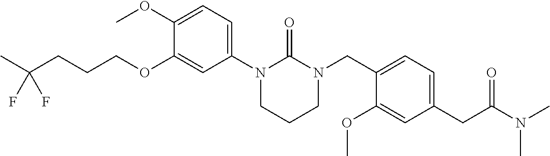 [SMILES: COc1cc(CC(=O)N(C)C)ccc1CN1CCCN(c2ccc(OC)c(OCCCC(C)(F)F)c2)C1=O] |
| CU0141 | 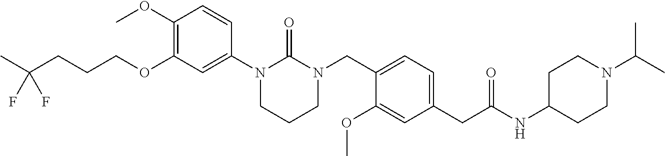 [SMILES: COc1cc(CC(=O)NC2CCN(C(C)C)CC2)ccc1CN1CCCN(c2ccc(OC)c(OCCCC(C)(F)F)c2)C1=O] |
| CU0142 | 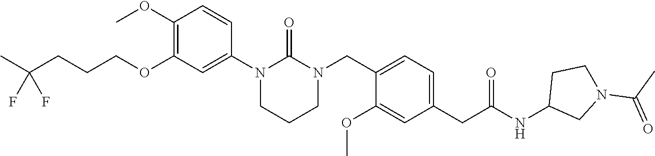 [SMILES: COc1cc(CC(=O)NC2CCN(C(C)=O)C2)ccc1CN1CCCN(c2ccc(OC)c(OCCCC(C)(F)F)c2)C1=O] |
| CU0143 | 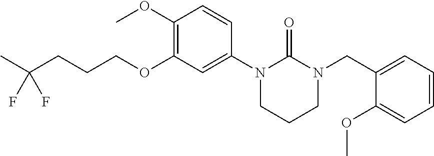 [SMILES: COc1ccccc1CN1CCCN(c2ccc(OC)c(OCCCC(C)(F)F)c2)C1=O] |
| CU0144 | 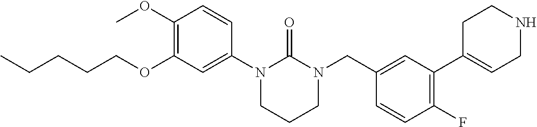 [SMILES: CCCCCOc1cc(N2CCCN(Cc3ccc(F)c(C4=CCNCC4)c3)C2=O)ccc1OC] |
| CU0145 | 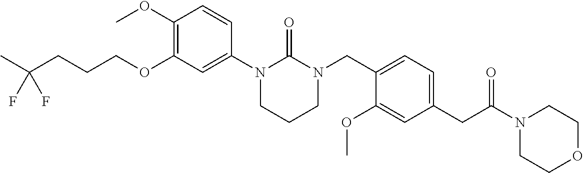 [SMILES: COc1cc(CC(=O)N2CCOCC2)ccc1CN1CCCN(c2ccc(OC)c(OCCCC(C)(F)F)c2)C1=O] |
| CU0146 | 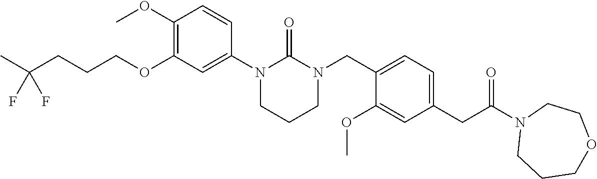 [SMILES: COc1cc(CC(=O)N2CCCOCC2)ccc1CN1CCCN(c2ccc(OC)c(OCCCC(C)(F)F)c2)C1=O] |
| CU0147 | 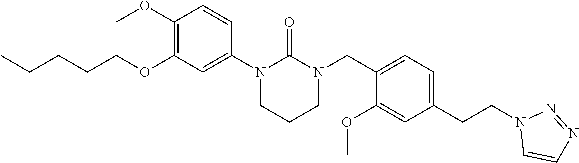 [SMILES: CCCCCOc1cc(N2CCCN(Cc3ccc(CCn4ccnn4)cc3OC)C2=O)ccc1OC] |
| CU0148 | 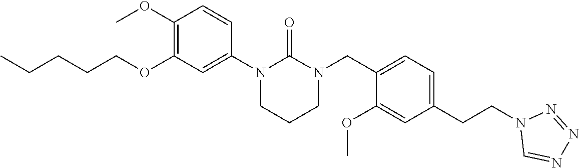 [SMILES: CCCCCOc1cc(N2CCCN(Cc3ccc(CCn4cnnn4)cc3OC)C2=O)ccc1OC] |
| CU0149 | 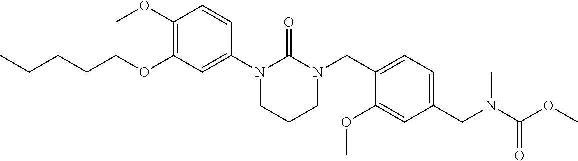 [SMILES: CCCCCOc1cc(N2CCCN(Cc3ccc(CN(C)C(=O)OC)cc3OC)C2=O)ccc1OC] |
| CU0150 | 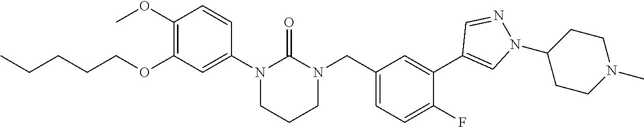 [SMILES: CCCCCOc1cc(N2CCCN(Cc3ccc(F)c(-c4cnn(C5CCN(C)CC5)c4)c3)C2=O)ccc1OC] |
| CU0151 | 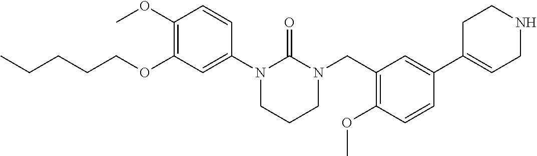 [SMILES: CCCCCOc1cc(N2CCCN(Cc3cc(C4=CCNCC4)ccc3OC)C2=O)ccc1OC] |
| CU0152 | 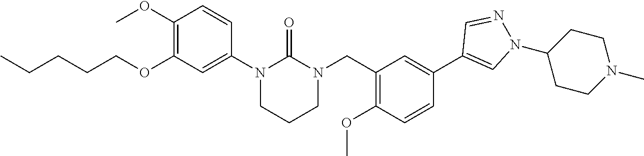 [SMILES: CCCCCOc1cc(N2CCCN(Cc3cc(-c4cnn(C5CCN(C)CC5)c4)ccc3OC)C2=O)ccc1OC] |
| CU0153 | 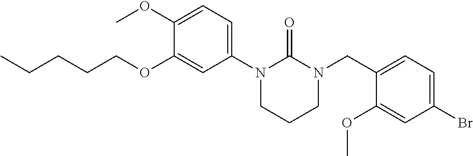 [SMILES: CCCCCOc1cc(N2CCCN(Cc3ccc(Br)cc3OC)C2=O)ccc1OC] |
| CU0154 | 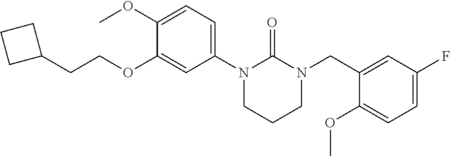 [SMILES: COc1ccc(F)cc1CN1CCCN(c2ccc(OC)c(OCCC3CCC3)c2)C1=O] |
| CU0155 | 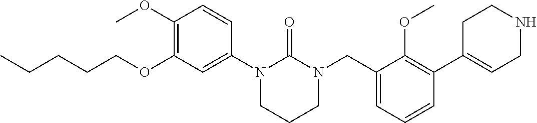 [SMILES: CCCCCOc1cc(N2CCCN(Cc3cccc(C4=CCNCC4)c3OC)C2=O)ccc1OC] |
| CU0156 | 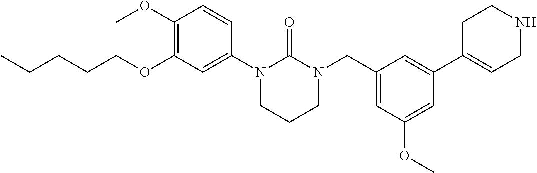 [SMILES: CCCCCOc1cc(N2CCCN(Cc3cc(OC)cc(C4=CCNCC4)c3)C2=O)ccc1OC] |
| CU0157 | 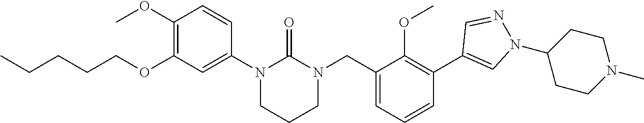 [SMILES: CCCCCOc1cc(N2CCCN(Cc3cccc(-c4cnn(C5CCN(C)CC5)c4)c3OC)C2=O)ccc1OC] |
| CU0158 | 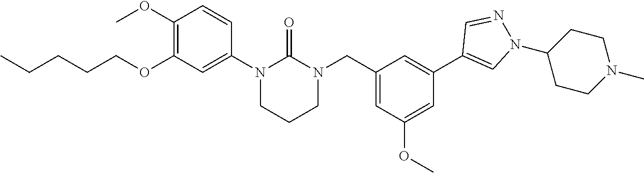 [SMILES: CCCCCOc1cc(N2CCCN(Cc3cc(OC)cc(-c4cnn(C5CCN(C)CC5)c4)c3)C2=O)ccc1OC] |
| CU0159 | 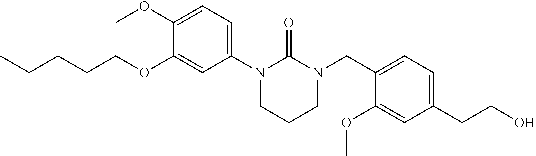 [SMILES: CCCCCOc1cc(N2CCCN(Cc3ccc(CCO)cc3OC)C2=O)ccc1OC] |
| CU0160 | 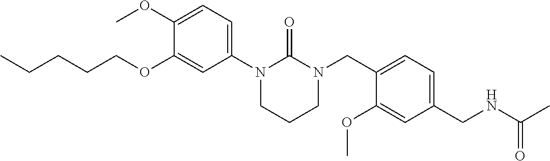 [SMILES: CCCCCOc1cc(N2CCCN(Cc3ccc(CNC(C)=O)cc3OC)C2=O)ccc1OC] |
| CU0161 | 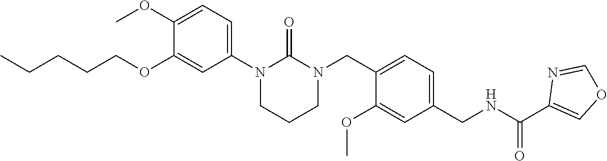 [SMILES: CCCCCOc1cc(N2CCCN(Cc3ccc(CNC(=O)c4cocn4)cc3OC)C2=O)ccc1OC] |
| CU0162 | 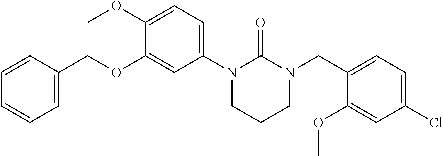 [SMILES: COc1cc(Cl)ccc1CN1CCCN(c2ccc(OC)c(OCc3ccccc3)c2)C1=O] |
| CU0163 | 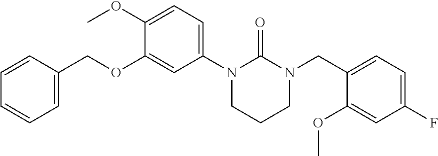 [SMILES: COc1cc(F)ccc1CN1CCCN(c2ccc(OC)c(OCc3ccccc3)c2)C1=O] |
| CU0164 | 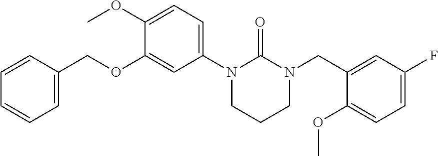 [SMILES: COc1ccc(F)cc1CN1CCCN(c2ccc(OC)c(OCc3ccccc3)c2)C1=O] |
| CU0165 | 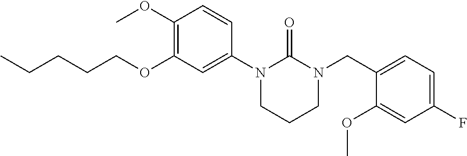 [SMILES: CCCCCOc1cc(N2CCCN(Cc3ccc(F)cc3OC)C2=O)ccc1OC] |
| CU0166 | 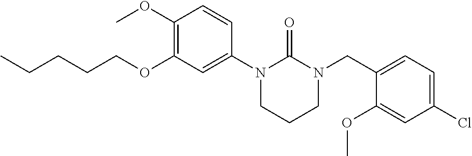 [SMILES: CCCCCOc1cc(N2CCCN(Cc3ccc(Cl)cc3OC)C2=O)ccc1OC] |
| CU0167 | 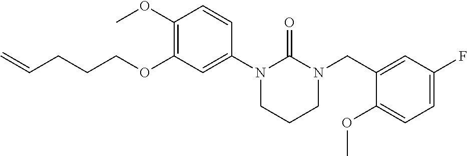 [SMILES: C=CCCCOc1cc(N2CCCN(Cc3cc(F)ccc3OC)C2=O)ccc1OC] |
| CU0168 | 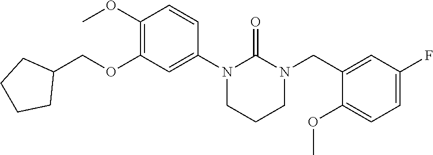 [SMILES: COc1ccc(F)cc1CN1CCCN(c2ccc(OC)c(OCC3CCCC3)c2)C1=O] |
| CU0169 | 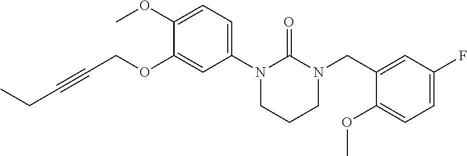 [SMILES: CCC#CCOc1cc(N2CCCN(Cc3cc(F)ccc3OC)C2=O)ccc1OC] |
| CU0170 | 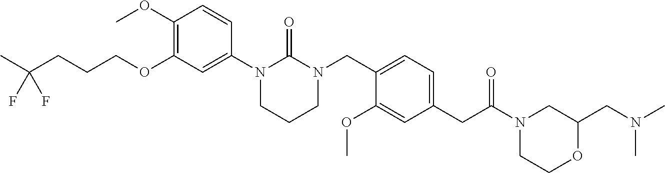 [SMILES: COc1cc(CC(=O)N2CCOC(CN(C)C)C2)ccc1CN1CCCN(c2ccc(OC)c(OCCCC(C)(F)F)c2)C1=O] |
| CU0171 | 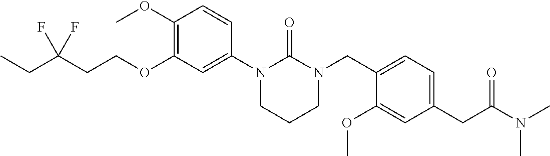 [SMILES: CCC(F)(F)CCOc1cc(N2CCCN(Cc3ccc(CC(=O)N(C)C)cc3OC)C2=O)ccc1OC] |
| CU0172 | 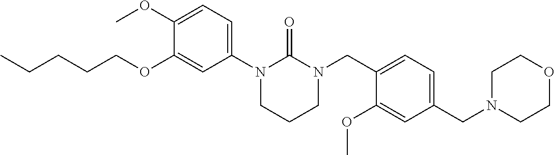 [SMILES: CCCCCOc1cc(N2CCCN(Cc3ccc(CN4CCOCC4)cc3OC)C2=O)ccc1OC] |
| CU0173 | 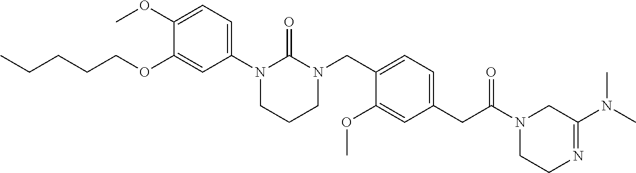 [SMILES: CCCCCOc1cc(N2CCCN(Cc3ccc(CC(=O)N4CCN=C(N(C)C)C4)cc3OC)C2=O)ccc1OC] |
| CU0174 | 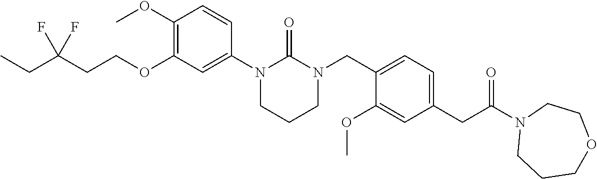 [SMILES: CCC(F)(F)CCOc1cc(N2CCCN(Cc3ccc(CC(=O)N4CCCOCC4)cc3OC)C2=O)ccc1OC] |
| CU0175 | 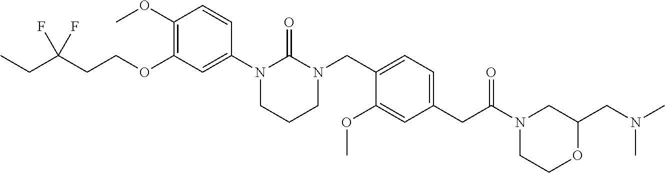 [SMILES: CCC(F)(F)CCOc1cc(N2CCCN(Cc3ccc(CC(=O)N4CCOC(CN(C)C)C4)cc3OC)C2=O)ccc1OC] |
| CU0176 | 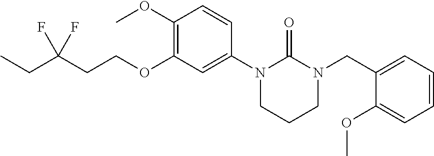 [SMILES: CCC(F)(F)CCOc1cc(N2CCCN(Cc3ccccc3OC)C2=O)ccc1OC] |
| CU0177 | 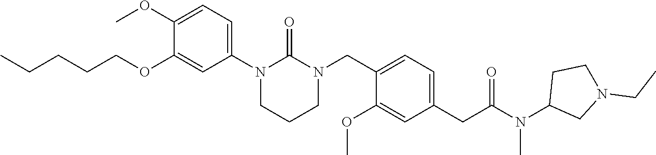 [SMILES: CCCCCOc1cc(N2CCCN(Cc3ccc(CC(=O)N(C)C4CCN(CC)C4)cc3OC)C2=O)ccc1OC] |
| CU0178 | 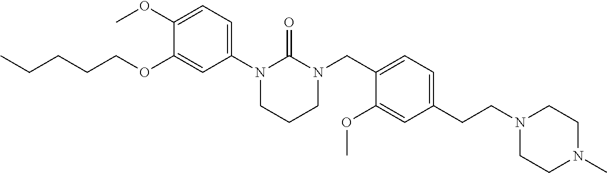 [SMILES: CCCCCOc1cc(N2CCCN(Cc3ccc(CCN4CCN(C)CC4)cc3OC)C2=O)ccc1OC] |
| CU0179 | 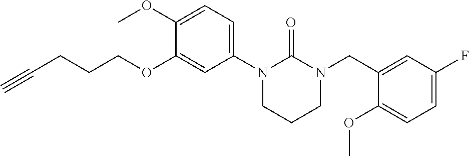 [SMILES: C#CCCCOc1cc(N2CCCN(Cc3cc(F)ccc3OC)C2=O)ccc1OC] |
| CU0180 | 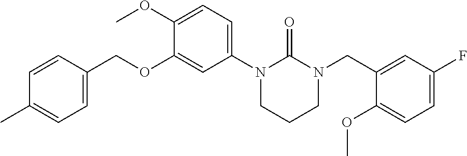 [SMILES: COc1ccc(F)cc1CN1CCCN(c2ccc(OC)c(OCc3ccc(C)cc3)c2)C1=O] |
| CU0181 | 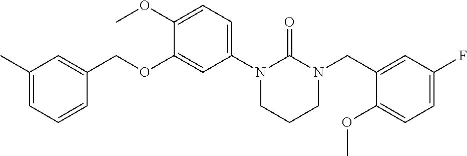 [SMILES: COc1ccc(F)cc1CN1CCCN(c2ccc(OC)c(OCc3cccc(C)c3)c2)C1=O] |
| CU0182 | 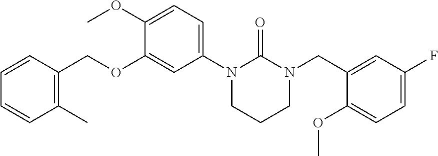 [SMILES: COc1ccc(F)cc1CN1CCCN(c2ccc(OC)c(OCc3ccccc3C)c2)C1=O] |
| CU0183 | 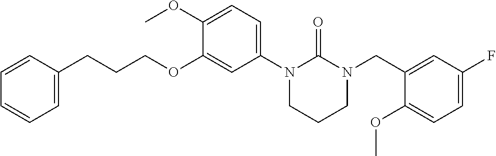 [SMILES: COc1ccc(F)cc1CN1CCCN(c2ccc(OC)c(OCCCc3ccccc3)c2)C1=O] |
| CU0184 | 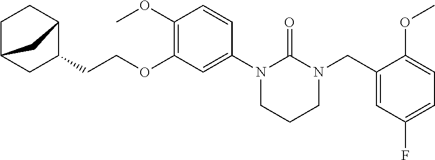 [SMILES: COc1ccc(F)cc1CN1CCCN(c2ccc(OC)c(OCC[C@@H]3C[C@@H]4CC[C@H]3C4)c2)C1=O] |
| CU0185 | 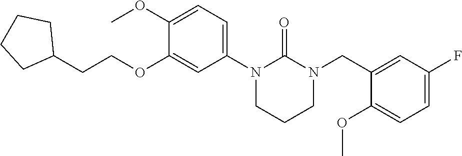 [SMILES: COc1ccc(F)cc1CN1CCCN(c2ccc(OC)c(OCCC3CCCC3)c2)C1=O] |
| CU0186 | 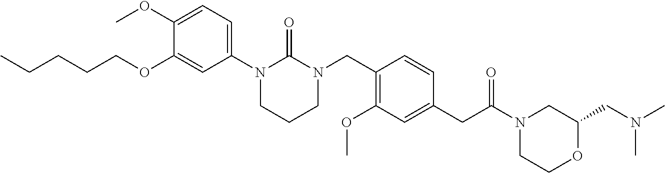 [SMILES: CCCCCOc1cc(N2CCCN(Cc3ccc(CC(=O)N4CCO[C@@H](CN(C)C)C4)cc3OC)C2=O)ccc1OC] |
| CU0187 | 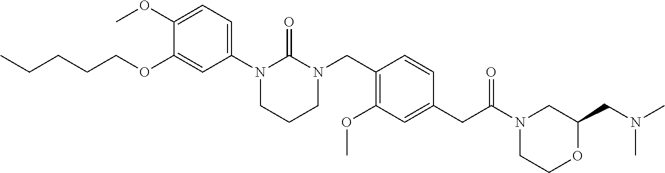 [SMILES: CCCCCOc1cc(N2CCCN(Cc3ccc(CC(=O)N4CCO[C@H](CN(C)C)C4)cc3OC)C2=O)ccc1OC] |
| CU0188 | 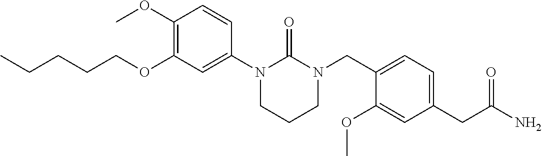 [SMILES: CCCCCOc1cc(N2CCCN(Cc3ccc(CC(N)=O)cc3OC)C2=O)ccc1OC] |
| CU0189 | 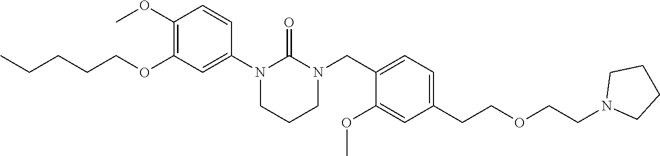 [SMILES: CCCCCOc1cc(N2CCCN(Cc3ccc(CCOCCN4CCCC4)cc3OC)C2=O)ccc1OC] |
| CU0190 | 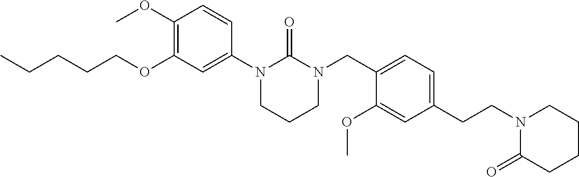 [SMILES: CCCCCOc1cc(N2CCCN(Cc3ccc(CCN4CCCCC4=O)cc3OC)C2=O)ccc1OC] |
| CU0191 | 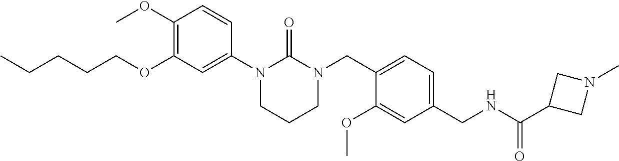 [SMILES: CCCCCOc1cc(N2CCCN(Cc3ccc(CNC(=O)C4CN(C)C4)cc3OC)C2=O)ccc1OC] |
| CU0192 | 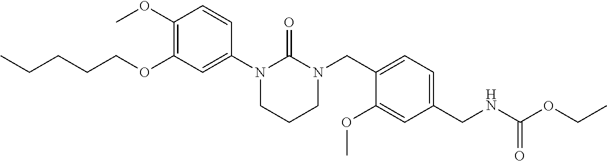 [SMILES: CCCCCOc1cc(N2CCCN(Cc3ccc(CNC(=O)OCC)cc3OC)C2=O)ccc1OC] |
| CU0193 | 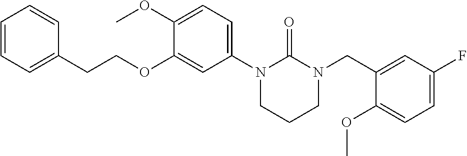 [SMILES: COc1ccc(F)cc1CN1CCCN(c2ccc(OC)c(OCCc3ccccc3)c2)C1=O] |
| CU0194 | 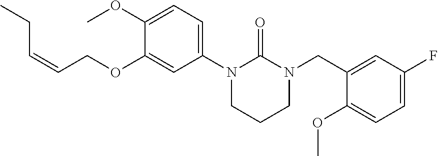 [SMILES: CC/C=C\COc1cc(N2CCCN(Cc3cc(F)ccc3OC)C2=O)ccc1OC] |
| CU0195 | 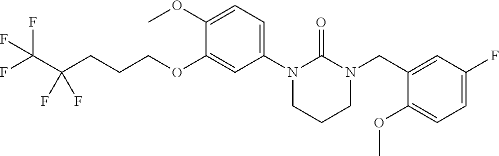 [SMILES: COc1ccc(F)cc1CN1CCCN(c2ccc(OC)c(OCCCC(F)(F)C(F)(F)F)c2)C1=O] |
| CU0196 | 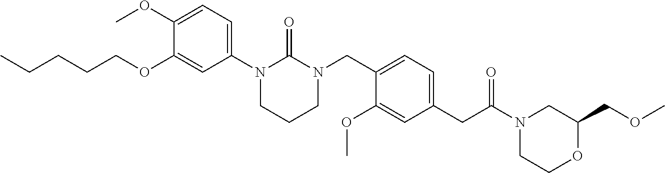 [SMILES: CCCCCOc1cc(N2CCCN(Cc3ccc(CC(=O)N4CCO[C@H](COC)C4)cc3OC)C2=O)ccc1OC] |
| CU0197 | 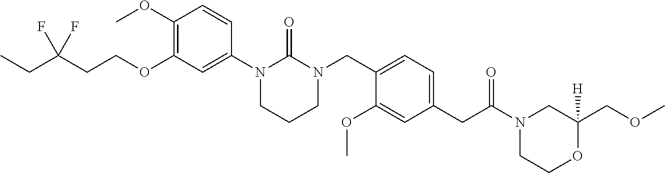 [SMILES: CCC(F)(F)CCOc1cc(N2CCCN(Cc3ccc(CC(=O)N4CCO[C@H](COC)C4)cc3OC)C2=O)ccc1OC] |
| CU0198 | 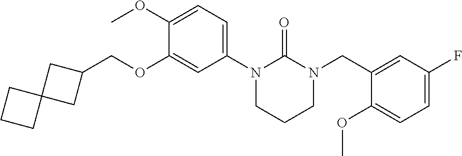 [SMILES: COc1ccc(F)cc1CN1CCCN(c2ccc(OC)c(OCC3CC4(CCC4)C3)c2)C1=O] |
| CU0199 | 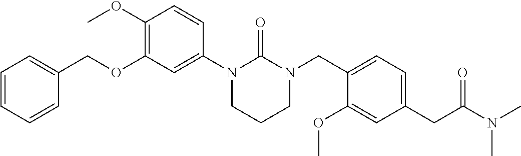 [SMILES: COc1cc(CC(=O)N(C)C)ccc1CN1CCCN(c2ccc(OC)c(OCc3ccccc3)c2)C1=O] |
| CU0200 | 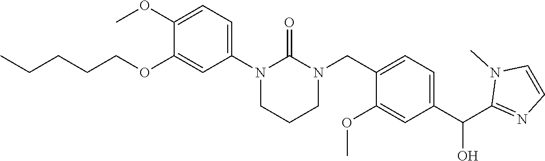 [SMILES: CCCCCOc1cc(N2CCCN(Cc3ccc(C(O)c4nccn4C)cc3OC)C2=O)ccc1OC] |
| CU0201 | 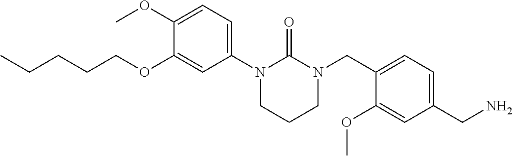 [SMILES: CCCCCOc1cc(N2CCCN(Cc3ccc(CN)cc3OC)C2=O)ccc1OC] |
| CU0202 | 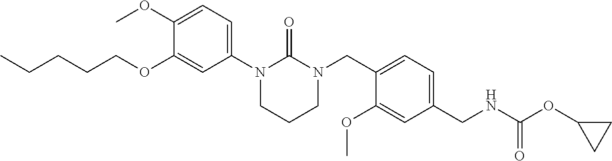 [SMILES: CCCCCOc1cc(N2CCCN(Cc3ccc(CNC(=O)OC4CC4)cc3OC)C2=O)ccc1OC] |
| CU0203 | 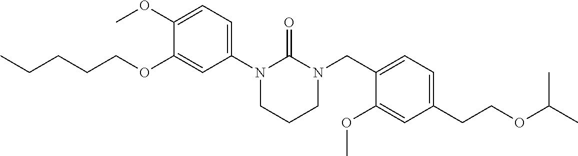 [SMILES: CCCCCOc1cc(N2CCCN(Cc3ccc(CCOC(C)C)cc3OC)C2=O)ccc1OC] |
| CU0204 | 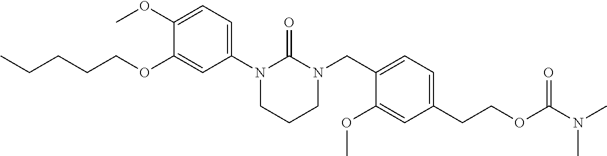 [SMILES: CCCCCOc1cc(N2CCCN(Cc3ccc(CCOC(=O)N(C)C)cc3OC)C2=O)ccc1OC] |
| CU0205 | 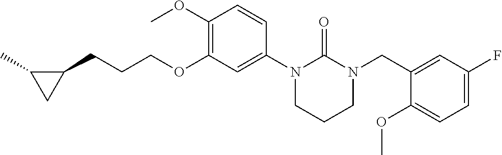 [SMILES: COc1ccc(F)cc1CN1CCCN(c2ccc(OC)c(OCCC[C@H]3C[C@@H]3C)c2)C1=O] |
| CU0206 | 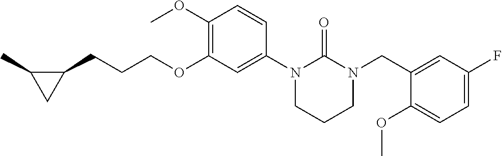 [SMILES: COc1ccc(F)cc1CN1CCCN(c2ccc(OC)c(OCCC[C@H]3C[C@H]3C)c2)C1=O] |
| CU0207 | 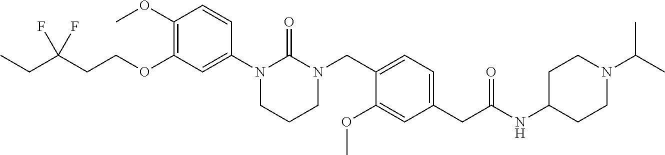 [SMILES: CCC(F)(F)CCOc1cc(N2CCCN(Cc3ccc(CC(=O)NC4CCN(C(C)C)CC4)cc3OC)C2=O)ccc1OC] |
| CU0208 | 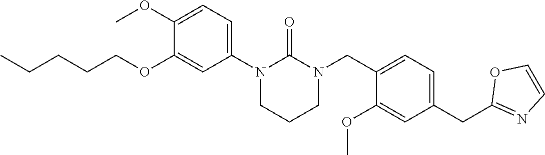 [SMILES: CCCCCOc1cc(N2CCCN(Cc3ccc(Cc4ncco4)cc3OC)C2=O)ccc1OC] |
| CU0209 | 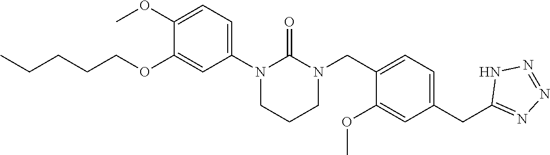 [SMILES: CCCCCOc1cc(N2CCCN(Cc3ccc(Cc4nnn[nH]4)cc3OC)C2=O)ccc1OC] |
| CU0210 | 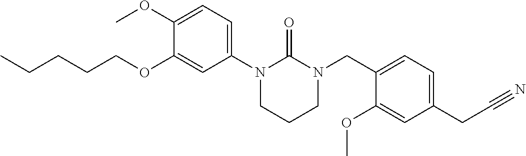 [SMILES: CCCCCOc1cc(N2CCCN(Cc3ccc(CC#N)cc3OC)C2=O)ccc1OC] |
| CU0211 | 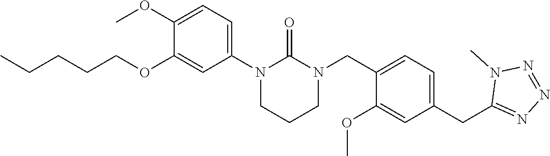 [SMILES: CCCCCOc1cc(N2CCCN(Cc3ccc(Cc4nnnn4C)cc3OC)C2=O)ccc1OC] |
| CU0212 | 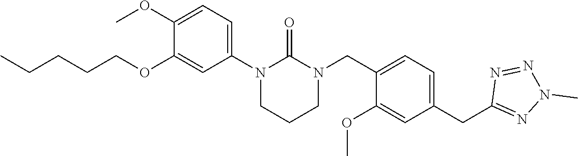 [SMILES: CCCCCOc1cc(N2CCCN(Cc3ccc(Cc4nnn(C)n4)cc3OC)C2=O)ccc1OC] |
| CU0213 | 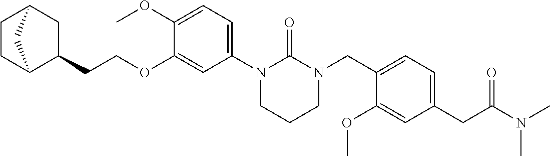 [SMILES: COc1cc(CC(=O)N(C)C)ccc1CN1CCCN(c2ccc(OC)c(OCC[C@@H]3C[C@@H]4CC[C@H]3C4)c2)C1=O] |
| CU0214 | 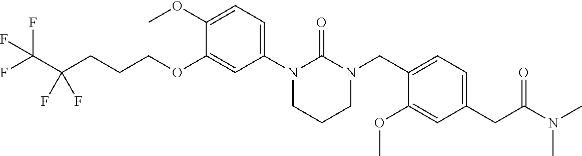 [SMILES: COc1cc(CC(=O)N(C)C)ccc1CN1CCCN(c2ccc(OC)c(OCCCC(F)(F)C(F)(F)F)c2)C1=O] |
| CU0215 | 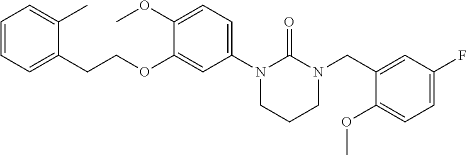 [SMILES: COc1ccc(F)cc1CN1CCCN(c2ccc(OC)c(OCCc3ccccc3C)c2)C1=O] |
| CU0216 | 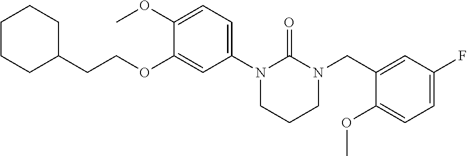 [SMILES: COc1ccc(F)cc1CN1CCCN(c2ccc(OC)c(OCCC3CCCCC3)c2)C1=O] |
| CU0217 | 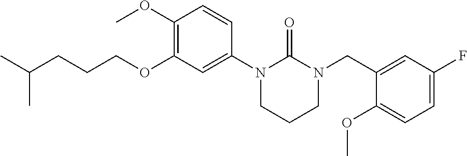 [SMILES: COc1ccc(F)cc1CN1CCCN(c2ccc(OC)c(OCCCC(C)C)c2)C1=O] |
| CU0218 | 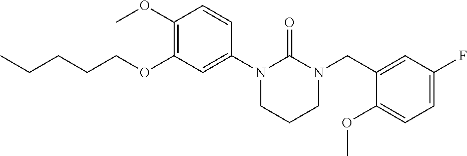 [SMILES: CCCCCOc1cc(N2CCCN(Cc3cc(F)ccc3OC)C2=O)ccc1OC] |
| CU0219 | 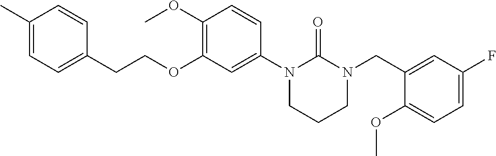 [SMILES: COc1ccc(F)cc1CN1CCCN(c2ccc(OC)c(OCCc3ccc(C)cc3)c2)C1=O] |
| CU0220 | 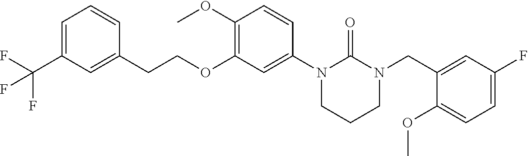 [SMILES: COc1ccc(F)cc1CN1CCCN(c2ccc(OC)c(OCCc3cccc(C(F)(F)F)c3)c2)C1=O] |
| CU0221 | 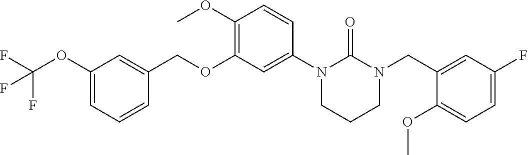 [SMILES: COc1ccc(F)cc1CN1CCCN(c2ccc(OC)c(OCc3cccc(OC(F)(F)F)c3)c2)C1=O] |
| CU0222 | 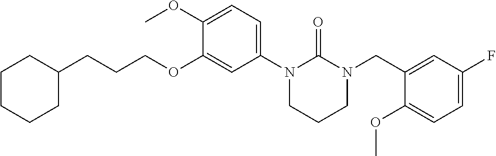 [SMILES: COc1ccc(F)cc1CN1CCCN(c2ccc(OC)c(OCCCC3CCCCC3)c2)C1=O] |
| CU0223 | 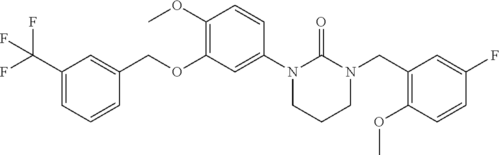 [SMILES: COc1ccc(F)cc1CN1CCCN(c2ccc(OC)c(OCc3cccc(C(F)(F)F)c3)c2)C1=O] |
| CU0224 | 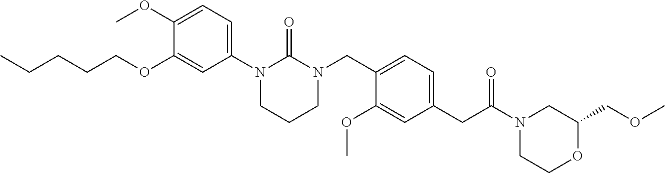 [SMILES: CCCCCOc1cc(N2CCCN(Cc3ccc(CC(=O)N4CCO[C@@H](COC)C4)cc3OC)C2=O)ccc1OC] |
| CU0225 | 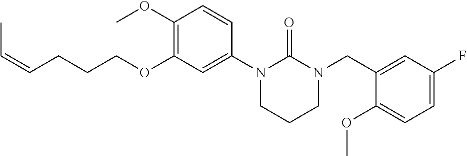 [SMILES: C/C=C\CCCOc1cc(N2CCCN(Cc3cc(F)ccc3OC)C2=O)ccc1OC] |
| CU0226 | 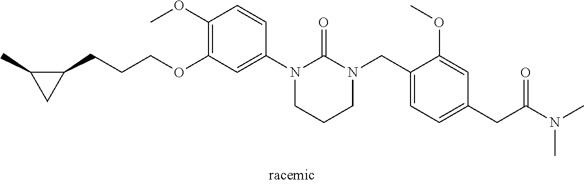 |
| CU0227 | 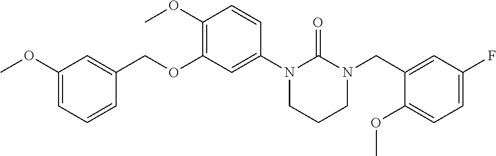 [SMILES: COc1cccc(COc2cc(N3CCCN(Cc4cc(F)ccc4OC)C3=O)ccc2OC)c1] |
| CU0228 | 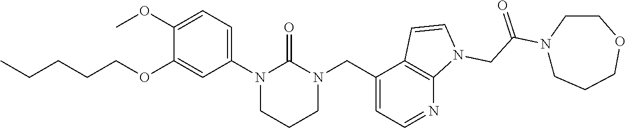 [SMILES: CCCCCOc1cc(N2CCCN(Cc3ccnc4c3ccn4CC(=O)N3CCCOCC3)C2=O)ccc1OC] |
| CU0229 | 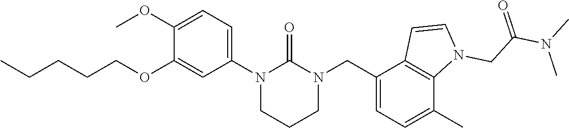 [SMILES: CCCCCOc1cc(N2CCCN(Cc3ccc(C)c4c3ccn4CC(=O)N(C)C)C2=O)ccc1OC] |
| CU0230 | 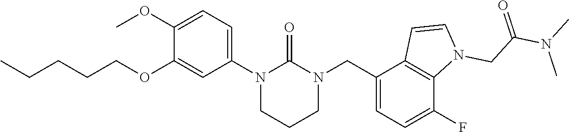 [SMILES: CCCCCOc1cc(N2CCCN(Cc3ccc(F)c4c3ccn4CC(=O)N(C)C)C2=O)ccc1OC] |
| CU0231 | 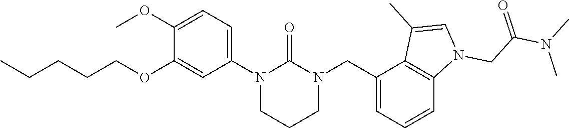 [SMILES: CCCCCOc1cc(N2CCCN(Cc3cccc4c3c(C)cn4CC(=O)N(C)C)C2=O)ccc1OC] |
| CU0232 | 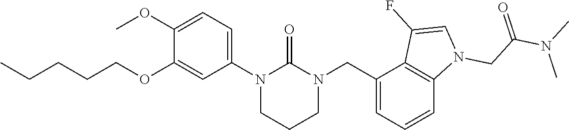 [SMILES: CCCCCOc1cc(N2CCCN(Cc3cccc4c3c(F)cn4CC(=O)N(C)C)C2=O)ccc1OC] |
| CU0233 | 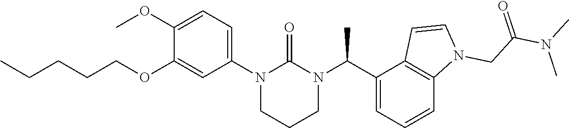 [SMILES: CCCCCOc1cc(N2CCCN([C@@H](C)c3cccc4c3ccn4CC(=O)N(C)C)C2=O)ccc1OC] |
| CU0234 | 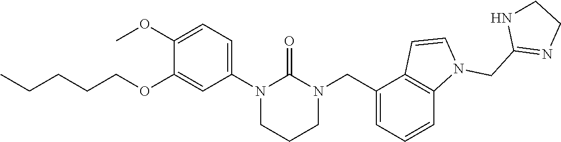 [SMILES: CCCCCOc1cc(N2CCCN(Cc3cccc4c3ccn4CC3=NCCN3)C2=O)ccc1OC] |
| CU0235 | 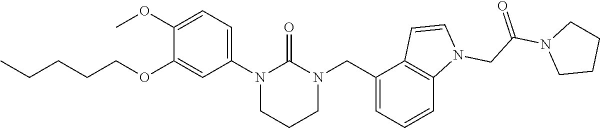 [SMILES: CCCCCOc1cc(N2CCCN(Cc3cccc4c3ccn4CC(=O)N3CCCC3)C2=O)ccc1OC] |
| CU0236 | 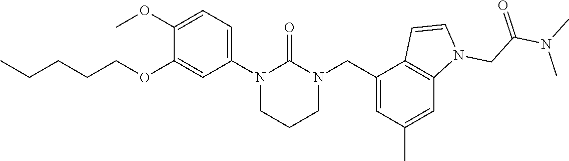 [SMILES: CCCCCOc1cc(N2CCCN(Cc3cc(C)cc4c3ccn4CC(=O)N(C)C)C2=O)ccc1OC] |
| CU0237 | 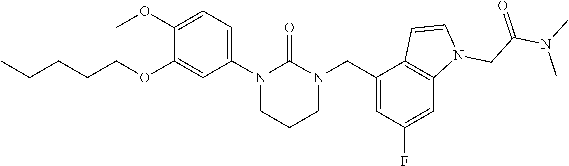 [SMILES: CCCCCOc1cc(N2CCCN(Cc3cc(F)cc4c3ccn4CC(=O)N(C)C)C2=O)ccc1OC] |
| CU0238 | 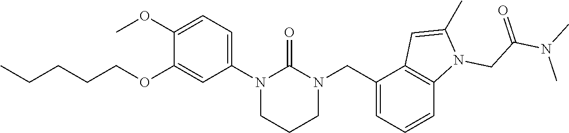 [SMILES: CCCCCOc1cc(N2CCCN(Cc3cccc4c3cc(C)n4CC(=O)N(C)C)C2=O)ccc1OC] |
| CU0239 | 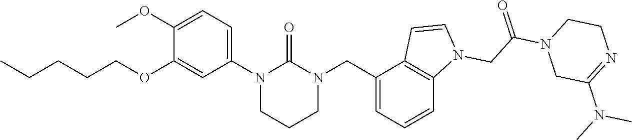 [SMILES: CCCCCOc1cc(N2CCCN(Cc3cccc4c3ccn4CC(=O)N3CCN=C(N(C)C)C3)C2=O)ccc1OC] |
| CU0240 | 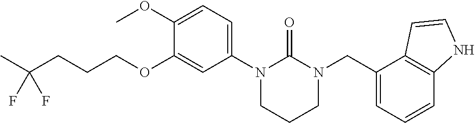 [SMILES: COc1ccc(N2CCCN(Cc3cccc4[nH]ccc34)C2=O)cc1OCCCC(C)(F)F] |
| CU0241 | 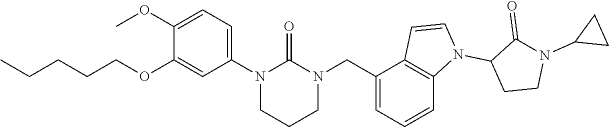 [SMILES: CCCCCOc1cc(N2CCCN(Cc3cccc4c3ccn4C3CCN(C4CC4)C3=O)C2=O)ccc1OC] |
| CU0242 | 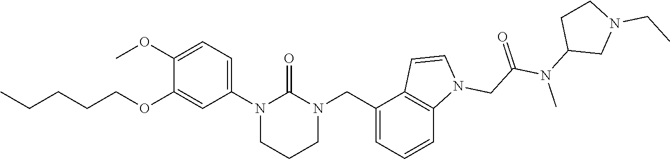 [SMILES: CCCCCOc1cc(N2CCCN(Cc3cccc4c3ccn4CC(=O)N(C)C3CCN(CC)C3)C2=O)ccc1OC] |
| CU0243 | 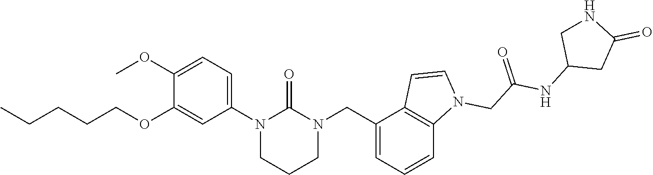 [SMILES: CCCCCOc1cc(N2CCCN(Cc3cccc4c3ccn4CC(=O)NC3CNC(=O)C3)C2=O)ccc1OC] |
| CU0244 | 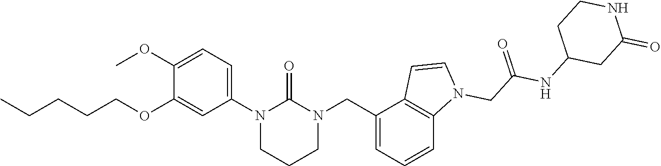 [SMILES: CCCCCOc1cc(N2CCCN(Cc3cccc4c3ccn4CC(=O)NC3CCNC(=O)C3)C2=O)ccc1OC] |
| CU0245 | 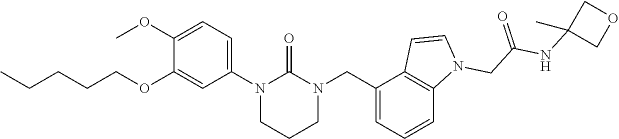 [SMILES: CCCCCOc1cc(N2CCCN(Cc3cccc4c3ccn4CC(=O)NC3(C)COC3)C2=O)ccc1OC] |
| CU0246 | 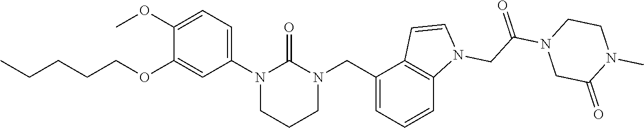 [SMILES: CCCCCOc1cc(N2CCCN(Cc3cccc4c3ccn4CC(=O)N3CCN(C)C(=O)C3)C2=O)ccc1OC] |
| CU0247 | 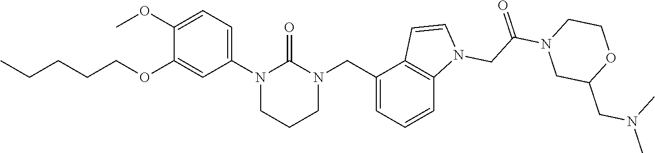 [SMILES: CCCCCOc1cc(N2CCCN(Cc3cccc4c3ccn4CC(=O)N3CCOC(CN(C)C)C3)C2=O)ccc1OC] |
| CU0248 | 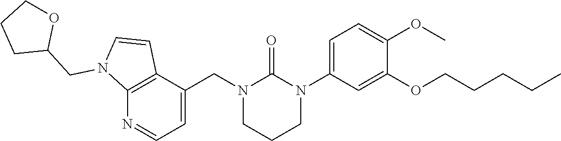 [SMILES: CCCCCOc1cc(N2CCCN(Cc3ccnc4c3ccn4CC3CCCO3)C2=O)ccc1OC] |
| CU0249 | 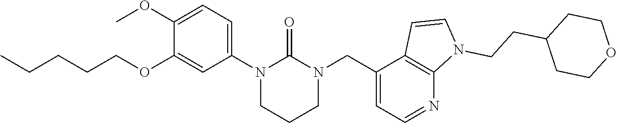 [SMILES: CCCCCOc1cc(N2CCCN(Cc3ccnc4c3ccn4CCC3CCOCC3)C2=O)ccc1OC] |
| CU0250 | 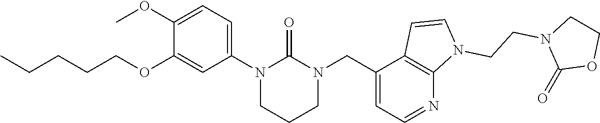 [SMILES: CCCCCOc1cc(N2CCCN(Cc3ccnc4c3ccn4CCN3CCOC3=O)C2=O)ccc1OC] |
| CU0251 | 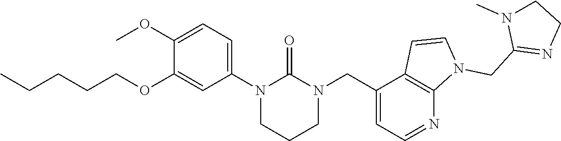 [SMILES: CCCCCOc1cc(N2CCCN(Cc3ccnc4c3ccn4CC3=NCCN3C)C2=O)ccc1OC] |
| CU0252 | 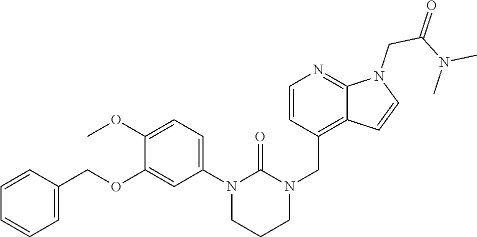 [SMILES: COc1ccc(N2CCCN(Cc3ccnc4c3ccn4CC(=O)N(C)C)C2=O)cc1OCc1ccccc1] |
| CU0253 | 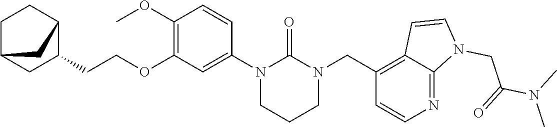 [SMILES: COc1ccc(N2CCCN(Cc3ccnc4c3ccn4CC(=O)N(C)C)C2=O)cc1OCC[C@@H]1C[C@@H]2CC[C@H]1C2] |
| CU0254 | 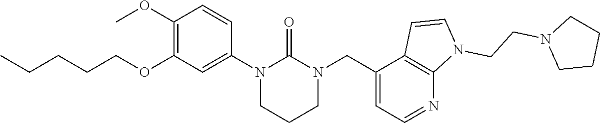 [SMILES: CCCCCOc1cc(N2CCCN(Cc3ccnc4c3ccn4CCN3CCCC3)C2=O)ccc1OC] |
| CU0255 | 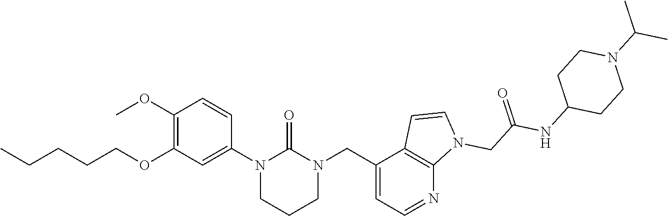 [SMILES: CCCCCOc1cc(N2CCCN(Cc3ccnc4c3ccn4CC(=O)NC3CCN(C(C)C)CC3)C2=O)ccc1OC] |
| CU0256 | 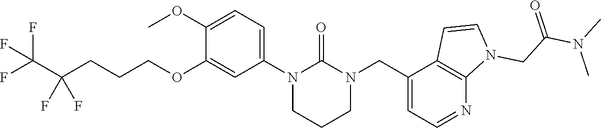 [SMILES: COc1ccc(N2CCCN(Cc3ccnc4c3ccn4CC(=O)N(C)C)C2=O)cc1OCCCC(F)(F)C(F)(F)F] |
| CU0257 | 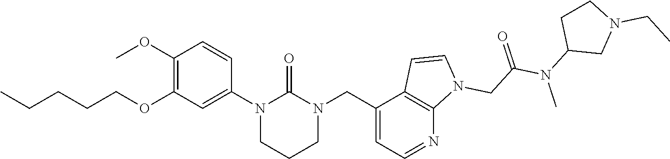 [SMILES: CCCCCOc1cc(N2CCCN(Cc3ccnc4c3ccn4CC(=O)N(C)C3CCN(CC)C3)C2=O)ccc1OC] |
| CU0258 | 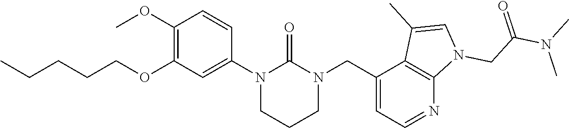 [SMILES: CCCCCOc1cc(N2CCCN(Cc3ccnc4c3c(C)cn4CC(=O)N(C)C)C2=O)ccc1OC] |
| CU0259 | 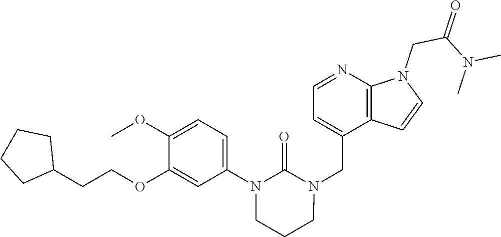 [SMILES: COc1ccc(N2CCCN(Cc3ccnc4c3ccn4CC(=O)N(C)C)C2=O)cc1OCCC1CCCC1] |
| CU0260 | 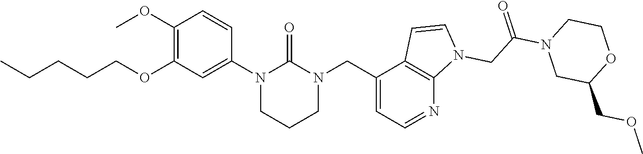 [SMILES: CCCCCOc1cc(N2CCCN(Cc3ccnc4c3ccn4CC(=O)N3CCO[C@@H](COC)C3)C2=O)ccc1OC] |
| CU0261 | 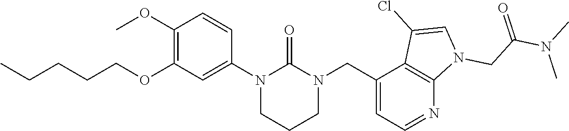 [SMILES: CCCCCOc1cc(N2CCCN(Cc3ccnc4c3c(Cl)cn4CC(=O)N(C)C)C2=O)ccc1OC] |
| CU0262 | 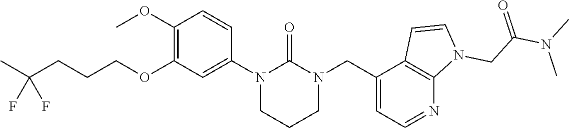 [SMILES: COc1ccc(N2CCCN(Cc3ccnc4c3ccn4CC(=O)N(C)C)C2=O)cc1OCCCC(C)(F)F] |
| CU0500 | 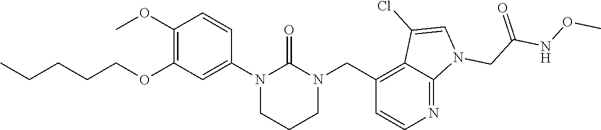 [SMILES: CCCCCOc1cc(N2CCCN(Cc3ccnc4c3c(Cl)cn4CC(=O)NOC)C2=O)ccc1OC] |
| CU0501 | 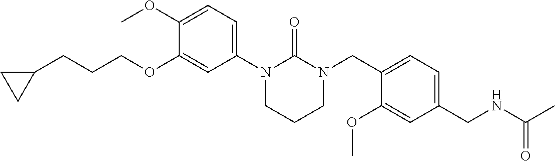 [SMILES: COc1cc(CNC(C)=O)ccc1CN1CCCN(c2ccc(OC)c(OCCCC3CC3)c2)C1=O] |
| CU0502 | 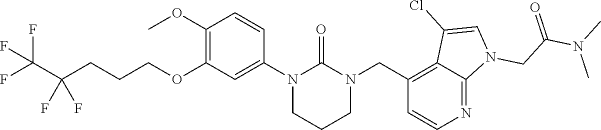 [SMILES: COc1ccc(N2CCCN(Cc3ccnc4c3c(Cl)cn4CC(=O)N(C)C)C2=O)cc1OCCCC(F)(F)C(F)(F)F] |
| CU0503 | 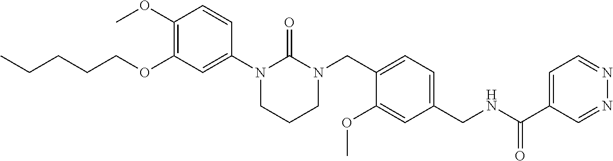 [SMILES: CCCCCOc1cc(N2CCCN(Cc3ccc(CNC(=O)c4ccnnc4)cc3OC)C2=O)ccc1OC] |
| CU0504 | 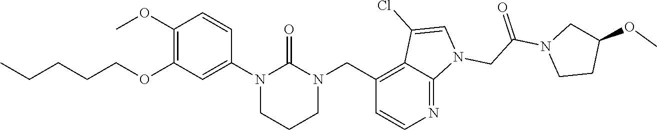 [SMILES: CCCCCOc1cc(N2CCCN(Cc3ccnc4c3c(Cl)cn4CC(=O)N3CC[C@H](OC)C3)C2=O)ccc1OC] |
| CU0505 | 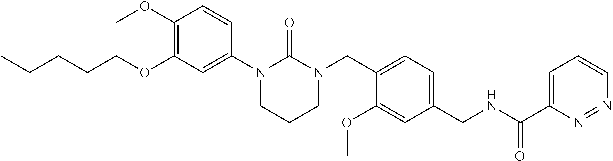 [SMILES: CCCCCOc1cc(N2CCCN(Cc3ccc(CNC(=O)c4cccnn4)cc3OC)C2=O)ccc1OC] |
| CU0506 | 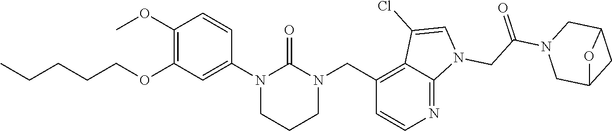 [SMILES: CCCCCOc1cc(N2CCCN(Cc3ccnc4c3c(Cl)cn4CC(=O)N3CC4CC(C3)O4)C2=O)ccc1OC] |
| CU0507 | 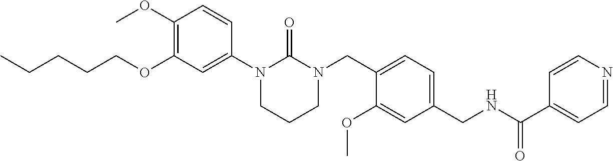 [SMILES: CCCCCOc1cc(N2CCCN(Cc3ccc(CNC(=O)c4ccncc4)cc3OC)C2=O)ccc1OC] |
| CU0508 | 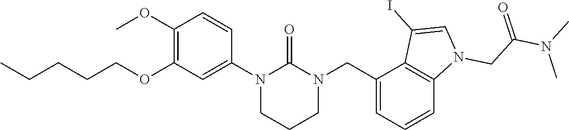 [SMILES: CCCCCOc1cc(N2CCCN(Cc3cccc4c3c(I)cn4CC(=O)N(C)C)C2=O)ccc1OC] |
| CU0509 | 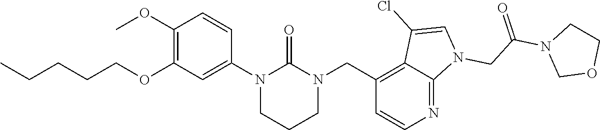 [SMILES: CCCCCOc1cc(N2CCCN(Cc3ccnc4c3c(Cl)cn4CC(=O)N3CCOC3)C2=O)ccc1OC] |
| CU0510 | 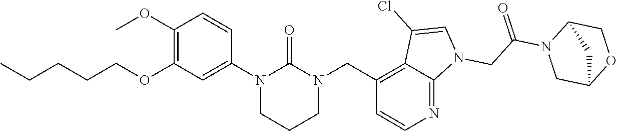 [SMILES: CCCCCOc1cc(N2CCCN(Cc3ccnc4c3c(Cl)cn4CC(=O)N3C[C@H]4C[C@@H]3CO4)C2=O)ccc1OC] |
| C00511 | 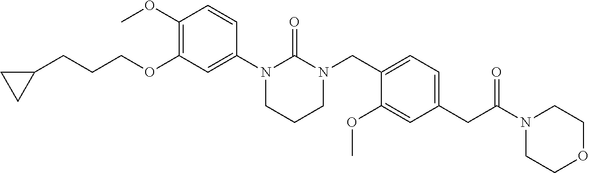 [SMILES: COc1cc(CC(=O)N2CCOCC2)ccc1CN1CCCN(c2ccc(OC)c(OCCCC3CC3)c2)C1=O] |
| CU0512 | 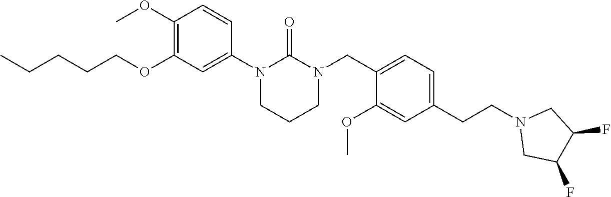 [SMILES: CCCCCOc1cc(N2CCCN(Cc3ccc(CCN4C[C@@H](F)[C@@H](F)C4)cc3OC)C2=O)ccc1OC] |
| CU0513 | 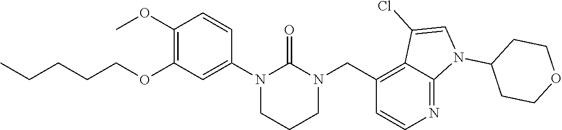 [SMILES: CCCCCOc1cc(N2CCCN(Cc3ccnc4c3c(Cl)cn4C3CCOCC3)C2=O)ccc1OC] |
| CU0514 | 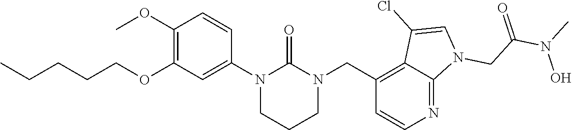 [SMILES: CCCCCOc1cc(N2CCCN(Cc3ccnc4c3c(Cl)cn4CC(=O)N(C)O)C2=O)ccc1OC] |
| CU0515 | 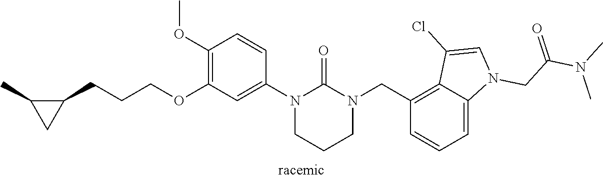 |
| CU0516 | 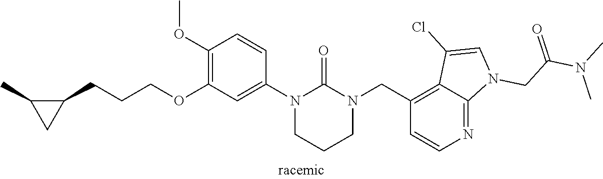 |
| CU0517 | 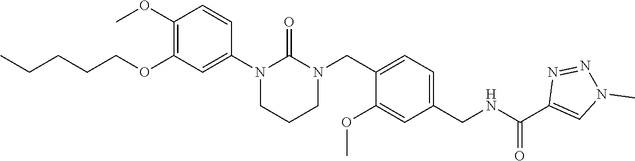 [SMILES: CCCCCOc1cc(N2CCCN(Cc3ccc(CNC(=O)c4cn(C)nn4)cc3OC)C2=O)ccc1OC] |
| CU0518 | 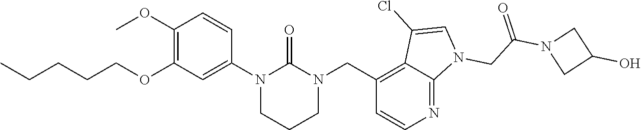 [SMILES: CCCCCOc1cc(N2CCCN(Cc3ccnc4c3c(Cl)cn4CC(=O)N3CC(O)C3)C2=O)ccc1OC] |
| CU0519 | 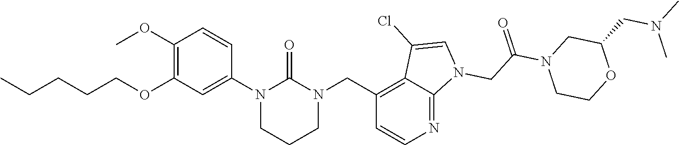 [SMILES: CCCCCOc1cc(N2CCCN(Cc3ccnc4c3c(Cl)cn4CC(=O)N3CCO[C@@H](CN(C)C)C3)C2=O)ccc1OC] |
| CU0520 | 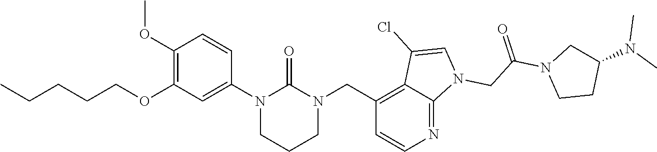 [SMILES: CCCCCOc1cc(N2CCCN(Cc3ccnc4c3c(Cl)cn4CC(=O)N3CC[C@@H](N(C)C)C3)C2=O)ccc1OC] |
| CU0521 | 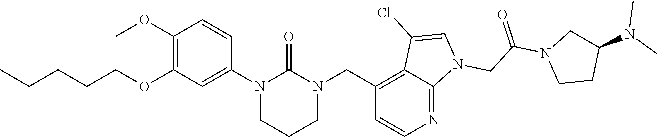 [SMILES: CCCCCOc1cc(N2CCCN(Cc3ccnc4c3c(Cl)cn4CC(=O)N3CC[C@H](N(C)C)C3)C2=O)ccc1OC] |
| CU0522 | 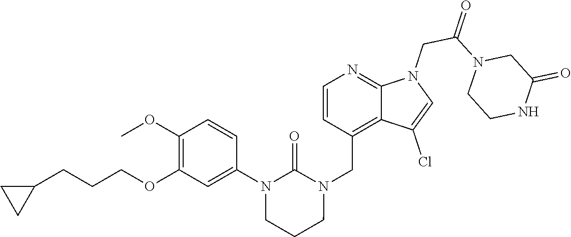 [SMILES: COc1ccc(N2CCCN(Cc3ccnc4c3c(Cl)cn4CC(=O)N3CCNC(=O)C3)C2=O)cc1OCCCC1CC1] |
| CU0523 | 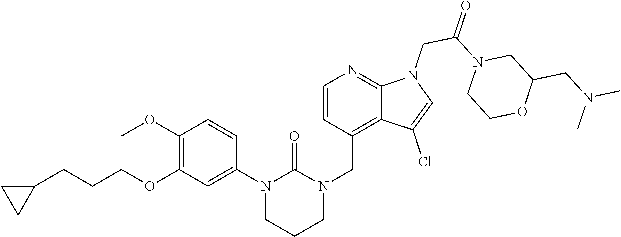 [SMILES: COc1ccc(N2CCCN(Cc3ccnc4c3c(Cl)cn4CC(=O)N3CCOC(CN(C)C)C3)C2=O)cc1OCCCC1CC1] |
| CU0524 | 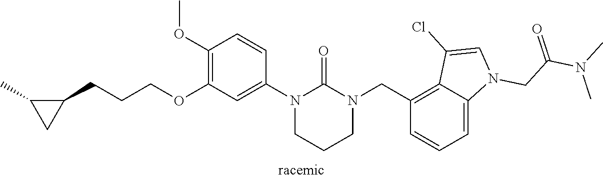 |
| CU0525 | 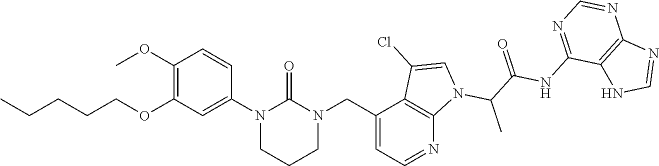 [SMILES: CCCCCOc1cc(N2CCCN(Cc3ccnc4c3c(Cl)cn4C(C)C(=O)Nc3ncnc4nc[nH]c34)C2=O)ccc1OC] |
| CU0526 | 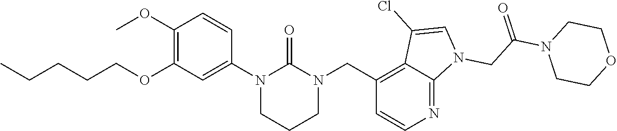 [SMILES: CCCCCOc1cc(N2CCCN(Cc3ccnc4c3c(Cl)cn4CC(=O)N3CCOCC3)C2=O)ccc1OC] |
| CU0527 | 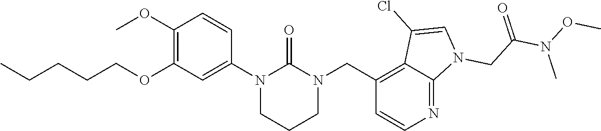 [SMILES: CCCCCOc1cc(N2CCCN(Cc3ccnc4c3c(Cl)cn4CC(=O)N(C)OC)C2=O)ccc1OC] |
| CU0528 | 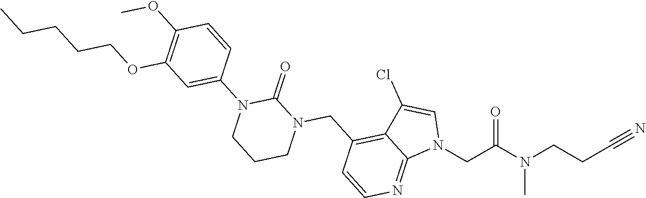 [SMILES: CCCCCOc1cc(N2CCCN(Cc3ccnc4c3c(Cl)cn4CC(=O)N(C)CCC#N)C2=O)ccc1OC] |
| CU0529 | 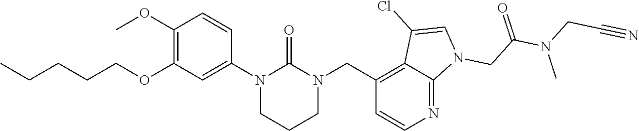 [SMILES: CCCCCOc1cc(N2CCCN(Cc3ccnc4c3c(Cl)cn4CC(=O)N(C)CC#N)C2=O)ccc1OC] |
| CU0530 | 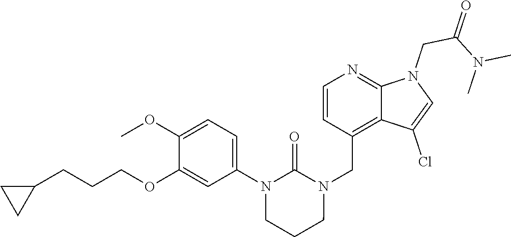 [SMILES: COc1ccc(N2CCCN(Cc3ccnc4c3c(Cl)cn4CC(=O)N(C)C)C2=O)cc1OCCCC1CC1] |
| CU0531 | 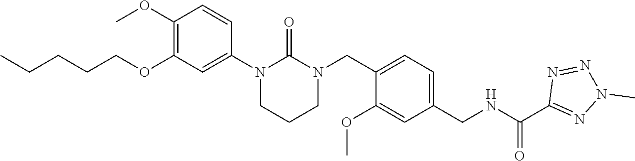 [SMILES: CCCCCOc1cc(N2CCCN(Cc3ccc(CNC(=O)c4nnn(C)n4)cc3OC)C2=O)ccc1OC] |
| CU0532 | 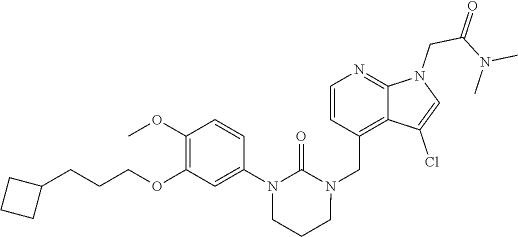 [SMILES: COc1ccc(N2CCCN(Cc3ccnc4c3c(Cl)cn4CC(=O)N(C)C)C2=O)cc1OCCCC1CCC1] |
| CU0533 | 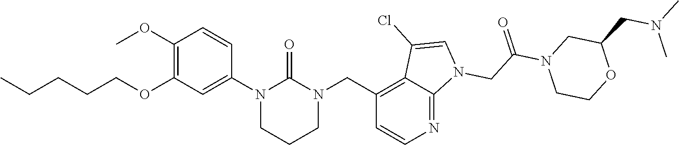 [SMILES: CCCCCOc1cc(N2CCCN(Cc3ccnc4c3c(Cl)cn4CC(=O)N3CCO[C@H](CN(C)C)C3)C2=O)ccc1OC] |
| CU0534 | 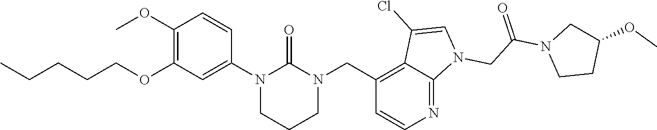 [SMILES: CCCCCOc1cc(N2CCCN(Cc3ccnc4c3c(Cl)cn4CC(=O)N3CC[C@@H](OC)C3)C2=O)ccc1OC] |
| CU0535 | 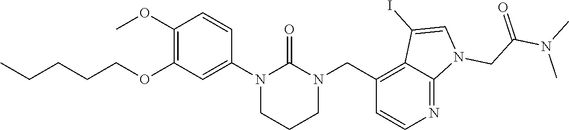 [SMILES: CCCCCOc1cc(N2CCCN(Cc3ccnc4c3c(I)cn4CC(=O)N(C)C)C2=O)ccc1OC] |
| CU0536 | 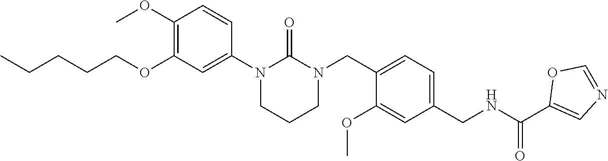 [SMILES: CCCCCOc1cc(N2CCCN(Cc3ccc(CNC(=O)c4cnco4)cc3OC)C2=O)ccc1OC] |
| CU0537 | 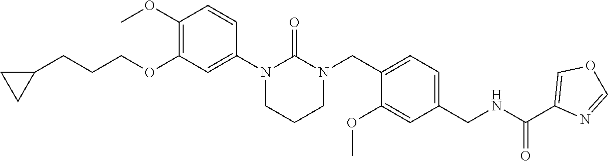 [SMILES: COc1cc(CNC(=O)c2cocn2)ccc1CN1CCCN(c2ccc(OC)c(OCCCC3CC3)c2)C1=O] |
| CU0538 | 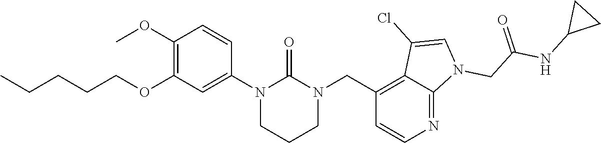 [SMILES: CCCCCOc1cc(N2CCCN(Cc3ccnc4c3c(Cl)cn4CC(=O)NC3CC3)C2=O)ccc1OC] |
| CU0539 | 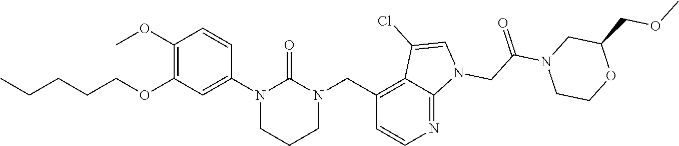 [SMILES: CCCCCOc1cc(N2CCCN(Cc3ccnc4c3c(Cl)cn4CC(=O)N3CCO[C@H](COC)C3)C2=O)ccc1OC] |
| CU0540 | 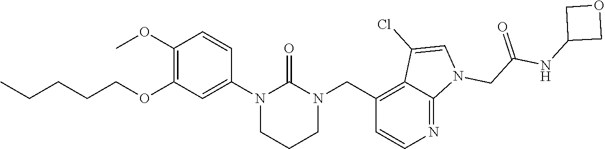 [SMILES: CCCCCOc1cc(N2CCCN(Cc3ccnc4c3c(Cl)cn4CC(=O)NC3COC3)C2=O)ccc1OC] |
| CU0541 | 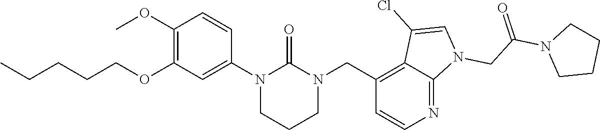 [SMILES: CCCCCOc1cc(N2CCCN(Cc3ccnc4c3c(Cl)cn4CC(=O)N3CCCC3)C2=O)ccc1OC] |
| CU0542 | 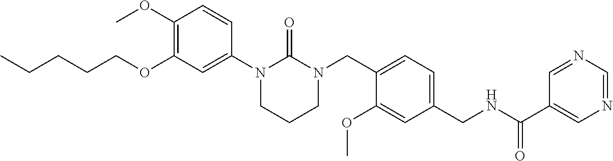 [SMILES: CCCCCOc1cc(N2CCCN(Cc3ccc(CNC(=O)c4cncnc4)cc3OC)C2=O)ccc1OC] |
| CU0543 | 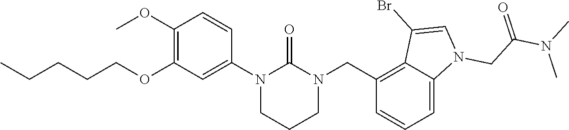 [SMILES: CCCCCOc1cc(N2CCCN(Cc3cccc4c3c(Br)cn4CC(=O)N(C)C)C2=O)ccc1OC] |
| CU0544 | 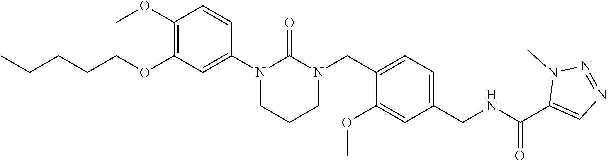 [SMILES: CCCCCOc1cc(N2CCCN(Cc3ccc(CNC(=O)c4cnnn4C)cc3OC)C2=O)ccc1OC] |
| CU0545 | 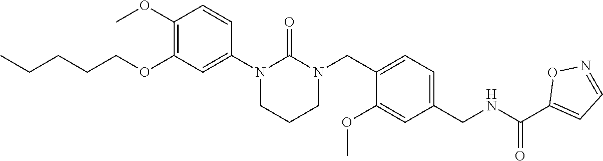 [SMILES: CCCCCOc1cc(N2CCCN(Cc3ccc(CNC(=O)c4ccno4)cc3OC)C2=O)ccc1OC] |
| CU0546 | 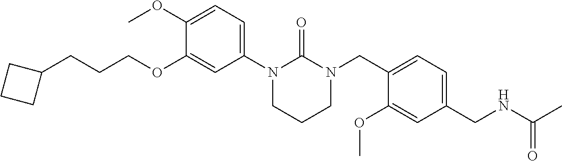 [SMILES: COc1cc(CNC(C)=O)ccc1CN1CCCN(c2ccc(OC)c(OCCCC3CCC3)c2)C1=O] |
| CU0547 | 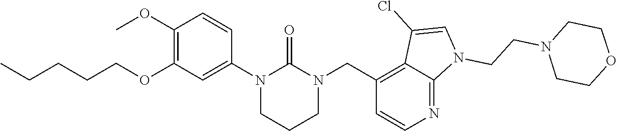 [SMILES: CCCCCOc1cc(N2CCCN(Cc3ccnc4c3c(Cl)cn4CCN3CCOCC3)C2=O)ccc1OC] |
| CU0548 | 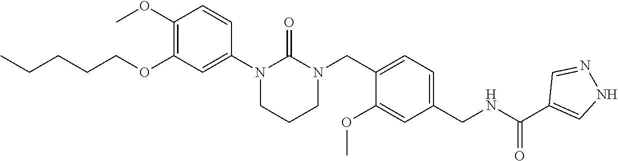 [SMILES: CCCCCOc1cc(N2CCCN(Cc3ccc(CNC(=O)c4cn[nH]c4)cc3OC)C2=O)ccc1OC] |
| CU0549 | 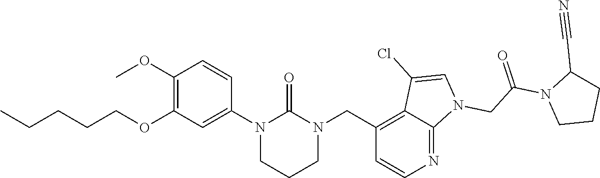 [SMILES: CCCCCOc1cc(N2CCCN(Cc3ccnc4c3c(Cl)cn4CC(=O)N3CCCC3C#N)C2=O)ccc1OC] |
| CU0550 | 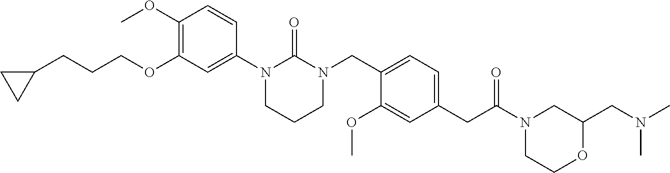 [SMILES: COc1cc(CC(=O)N2CCOC(CN(C)C)C2)ccc1CN1CCCN(c2ccc(OC)c(OCCCC3CC3)c2)C1=O] |
| CU0551 | 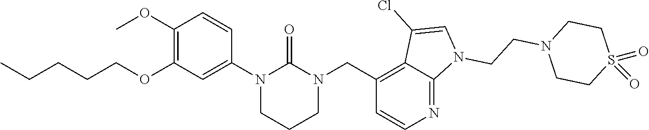 [SMILES: CCCCCOc1cc(N2CCCN(Cc3ccnc4c3c(Cl)cn4CCN3CCS(=O)(=O)CC3)C2=O)ccc1OC] |
| CU0552 | 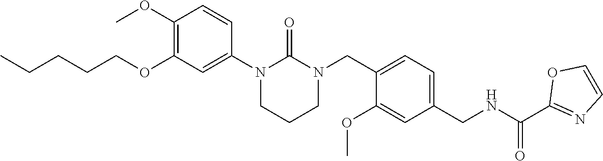 [SMILES: CCCCCOc1cc(N2CCCN(Cc3ccc(CNC(=O)c4ncco4)cc3OC)C2=O)ccc1OC] |
| CU0553 | 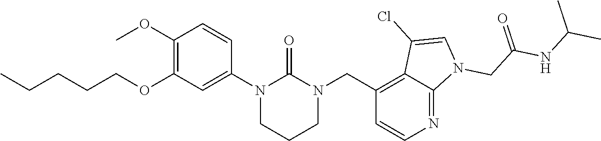 [SMILES: CCCCCOc1cc(N2CCCN(Cc3ccnc4c3c(Cl)cn4CC(=O)NC(C)C)C2=O)ccc1OC] |
| CU0554 | 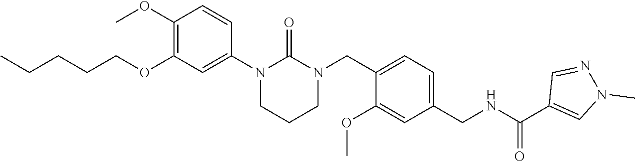 [SMILES: CCCCCOc1cc(N2CCCN(Cc3ccc(CNC(=O)c4cnn(C)c4)cc3OC)C2=O)ccc1OC] |
| CU0555 | 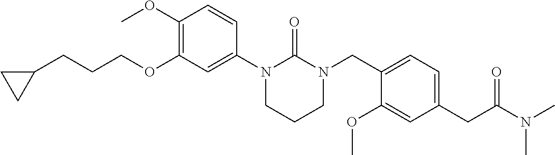 [SMILES: COc1cc(CC(=O)N(C)C)ccc1CN1CCCN(c2ccc(OC)c(OCCCC3CC3)c2)C1=O] |
| CU0556 | 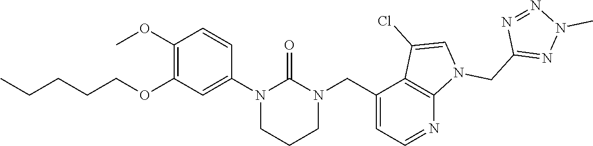 [SMILES: CCCCCOc1cc(N2CCCN(Cc3ccnc4c3c(Cl)cn4Cc3nnn(C)n3)C2=O)ccc1OC] |
| CU0557 | 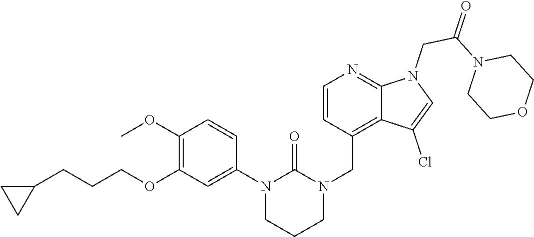 [SMILES: COc1ccc(N2CCCN(Cc3ccnc4c3c(Cl)cn4CC(=O)N3CCOCC3)C2=O)cc1OCCCC1CC1] |
| CU0558 | 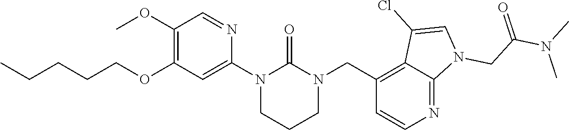 [SMILES: CCCCCOc1cc(N2CCCN(Cc3ccnc4c3c(Cl)cn4CC(=O)N(C)C)C2=O)ncc1OC] |
| CU0559 | 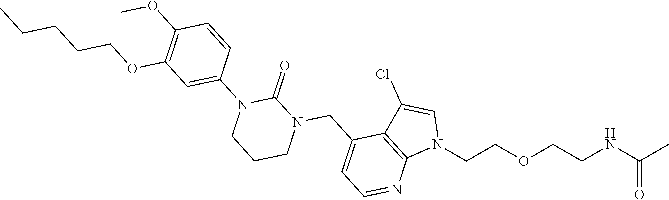 [SMILES: CCCCCOc1cc(N2CCCN(Cc3ccnc4c3c(Cl)cn4CCOCCNC(C)=O)C2=O)ccc1OC] |
| CU0560 | 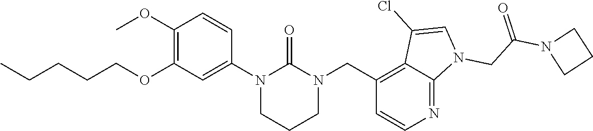 [SMILES: CCCCCOc1cc(N2CCCN(Cc3ccnc4c3c(Cl)cn4CC(=O)N3CCC3)C2=O)ccc1OC] |
| CU0561 | 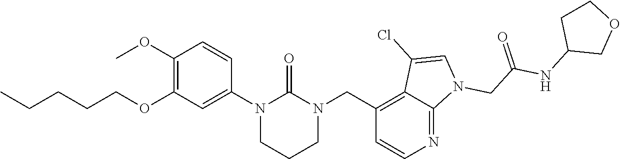 [SMILES: CCCCCOc1cc(N2CCCN(Cc3ccnc4c3c(Cl)cn4CC(=O)NC3CCOC3)C2=O)ccc1OC] |
| CU0562 | 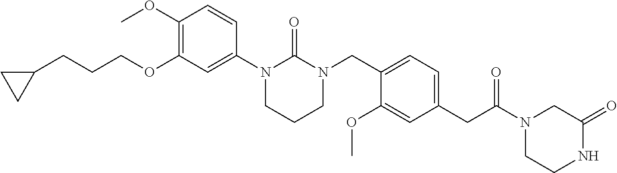 [SMILES: COc1cc(CC(=O)N2CCNC(=O)C2)ccc1CN1CCCN(c2ccc(OC)c(OCCCC3CC3)c2)C1=O] |
| CU0563 | 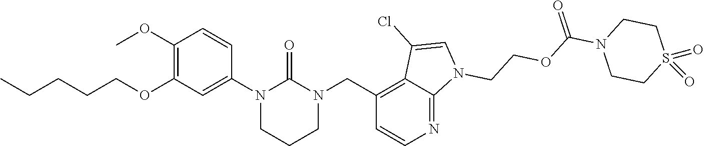 [SMILES: CCCCCOc1cc(N2CCCN(Cc3ccnc4c3c(Cl)cn4CCOC(=O)N3CCS(=O)(=O)CC3)C2=O)ccc1OC] |
| CU0564 | 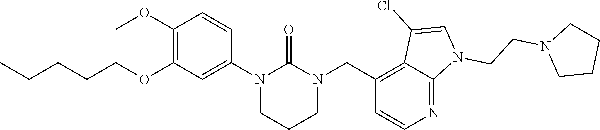 [SMILES: CCCCCOc1cc(N2CCCN(Cc3ccnc4c3c(Cl)cn4CCN3CCCC3)C2=O)ccc1OC] |
| CU0565 | 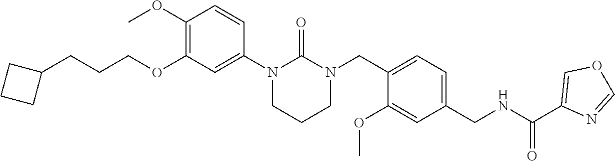 [SMILES: COc1cc(CNC(=O)c2cocn2)ccc1CN1CCCN(c2ccc(OC)c(OCCCC3CCC3)c2)C1=O] |
| CU0566 | 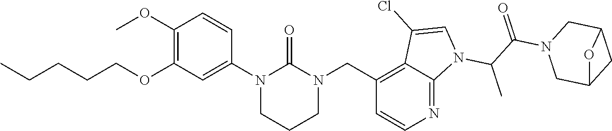 [SMILES: CCCCCOc1cc(N2CCCN(Cc3ccnc4c3c(Cl)cn4C(C)C(=O)N3CC4CC(C3)O4)C2=O)ccc1OC] |
| CU0567 | 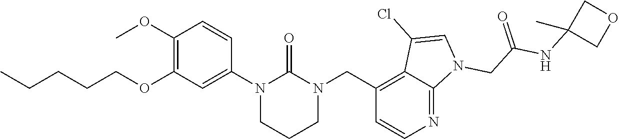 [SMILES: CCCCCOc1cc(N2CCCN(Cc3ccnc4c3c(Cl)cn4CC(=O)NC3(C)COC3)C2=O)ccc1OC] |
| CU0568 | 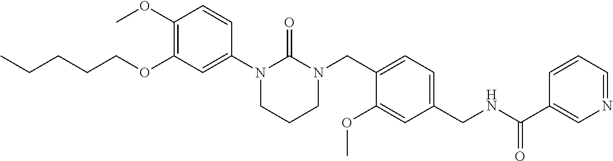 [SMILES: CCCCCOc1cc(N2CCCN(Cc3ccc(CNC(=O)c4cccnc4)cc3OC)C2=O)ccc1OC] |
| CU0569 | 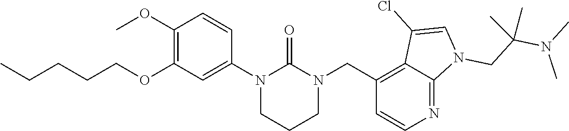 [SMILES: CCCCCOc1cc(N2CCCN(Cc3ccnc4c3c(Cl)cn4CC(C)(C)N(C)C)C2=O)ccc1OC] |
| CU0570 | 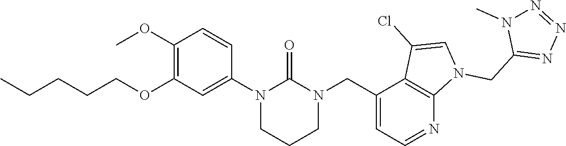 [SMILES: CCCCCOc1cc(N2CCCN(Cc3ccnc4c3c(Cl)cn4Cc3nnnn3C)C2=O)ccc1OC] |
| CU0571 | 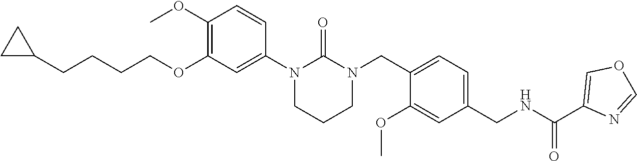 [SMILES: COc1cc(CNC(=O)c2cocn2)ccc1CN1CCCN(c2ccc(OC)c(OCCCCC3CC3)c2)C1=O] |
| CU0572 | 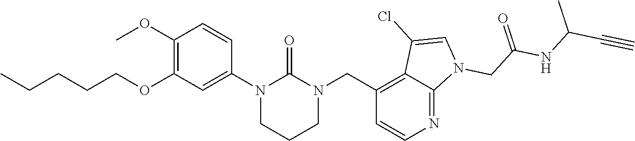 [SMILES: C#CC(C)NC(=O)Cn1cc(Cl)c2c(CN3CCCN(c4ccc(OC)c(OCCCCC)c4)C3=O)ccnc21] |
| CU0573 | 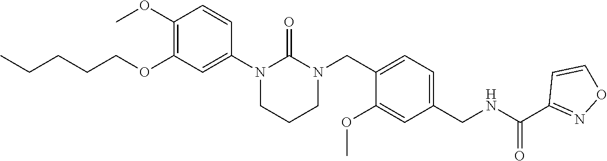 [SMILES: CCCCCOc1cc(N2CCCN(Cc3ccc(CNC(=O)c4ccon4)cc3OC)C2=O)ccc1OC] |
| CU0574 | 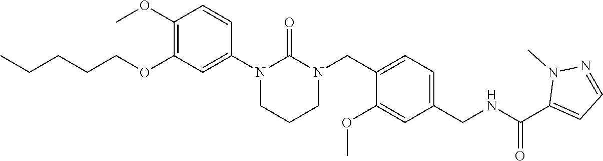 [SMILES: CCCCCOc1cc(N2CCCN(Cc3ccc(CNC(=O)c4ccnn4C)cc3OC)C2=O)ccc1OC] |
| CU0575 | 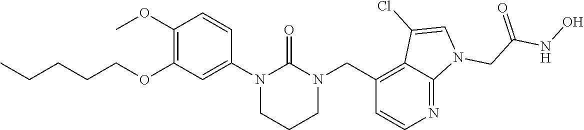 [SMILES: CCCCCOc1cc(N2CCCN(Cc3ccnc4c3c(Cl)cn4CC(=O)NO)C2=O)ccc1OC] |
| CU0576 | 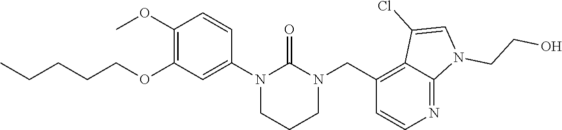 [SMILES: CCCCCOc1cc(N2CCCN(Cc3ccnc4c3c(Cl)cn4CCO)C2=O)ccc1OC] |
| CU0577 | 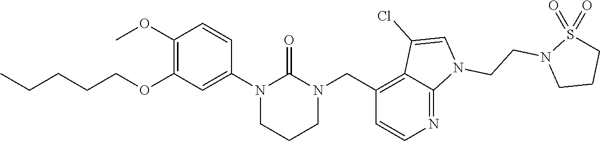 [SMILES: CCCCCOc1cc(N2CCCN(Cc3ccnc4c3c(Cl)cn4CCN3CCCS3(=O)=O)C2=O)ccc1OC] |
| CU0578 | 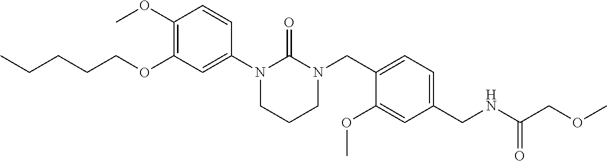 [SMILES: CCCCCOc1cc(N2CCCN(Cc3ccc(CNC(=O)COC)cc3OC)C2=O)ccc1OC] |
| CU0579 | 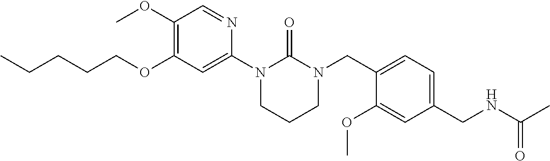 [SMILES: CCCCCOc1cc(N2CCCN(Cc3ccc(CNC(C)=O)cc3OC)C2=O)ncc1OC] |
| CU0580 | 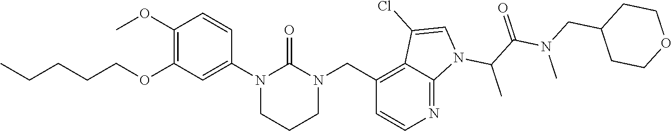 [SMILES: CCCCCOc1cc(N2CCCN(Cc3ccnc4c3c(Cl)cn4C(C)C(=O)N(C)CC3CCOCC3)C2=O)ccc1OC] |
| CU0581 | 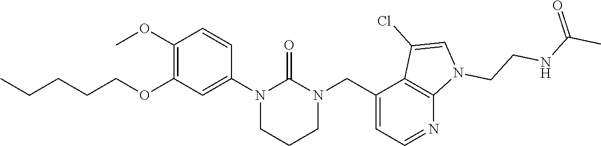 [SMILES: CCCCCOc1cc(N2CCCN(Cc3ccnc4c3c(Cl)cn4CCNC(C)=O)C2=O)ccc1OC] |
| CU0582 | 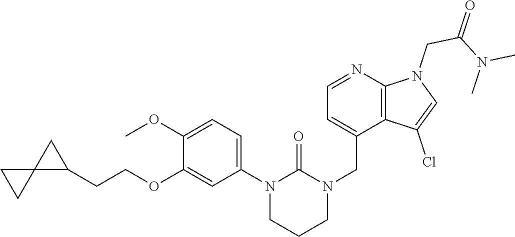 [SMILES: COc1ccc(N2CCCN(Cc3ccnc4c3c(Cl)cn4CC(=O)N(C)C)C2=O)cc1OCCC1CC12CC2] |
| CU0583 | 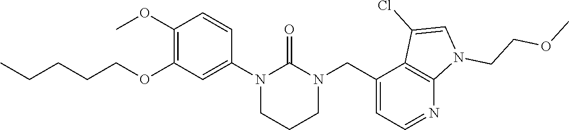 [SMILES: CCCCCOc1cc(N2CCCN(Cc3ccnc4c3c(Cl)cn4CCOC)C2=O)ccc1OC] |
| CU0584 | 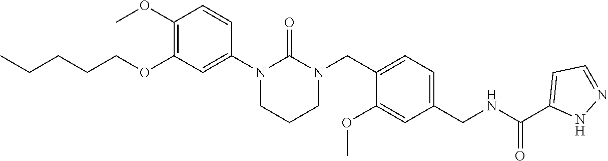 [SMILES: CCCCCOc1cc(N2CCCN(Cc3ccc(CNC(=O)c4ccn[nH]4)cc3OC)C2=O)ccc1OC] |
| CU0585 | 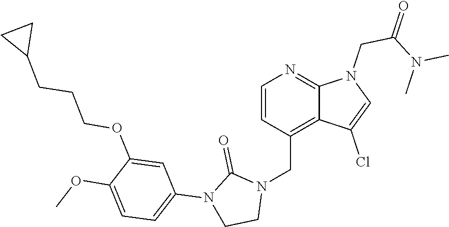 [SMILES: COc1ccc(N2CCN(Cc3ccnc4c3c(Cl)cn4CC(=O)N(C)C)C2=O)cc1OCCCC1CC1] |
| CU0586 | 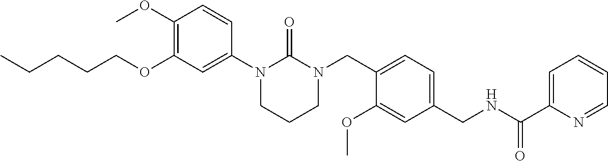 [SMILES: CCCCCOc1cc(N2CCCN(Cc3ccc(CNC(=O)c4ccccn4)cc3OC)C2=O)ccc1OC] |
| CU0587 | 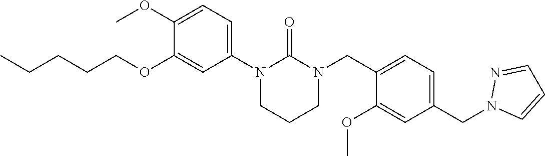 [SMILES: CCCCCOc1cc(N2CCCN(Cc3ccc(Cn4cccn4)cc3OC)C2=O)ccc1OC] |
| CU0588 | 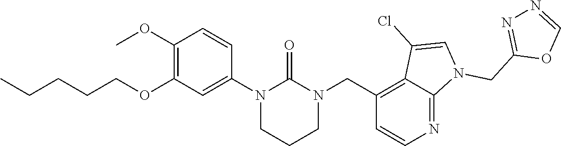 [SMILES: CCCCCOc1cc(N2CCCN(Cc3ccnc4c3c(Cl)cn4Cc3nnco3)C2=O)ccc1OC] |
| CU0589 | 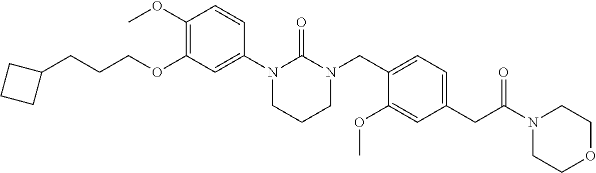 [SMILES: COc1cc(CC(=O)N2CCOCC2)ccc1CN1CCCN(c2ccc(OC)c(OCCCC3CCC3)c2)C1=O] |
| CU0590 | 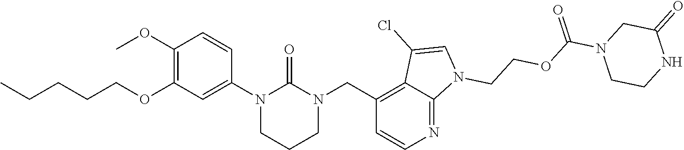 [SMILES: CCCCCOc1cc(N2CCCN(Cc3ccnc4c3c(Cl)cn4CCOC(=O)N3CCNC(=O)C3)C2=O)ccc1OC] |
| CU0591 | 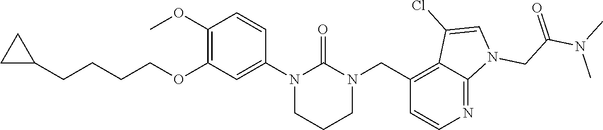 [SMILES: COc1ccc(N2CCCN(Cc3ccnc4c3c(Cl)cn4CC(=O)N(C)C)C2=O)cc1OCCCCC1CC1] |
| CU0592 | 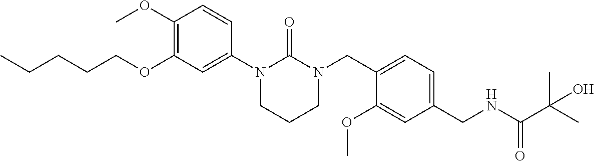 [SMILES: CCCCCOc1cc(N2CCCN(Cc3ccc(CNC(=O)C(C)(C)O)cc3OC)C2=O)ccc1OC] |
| CU0593 | 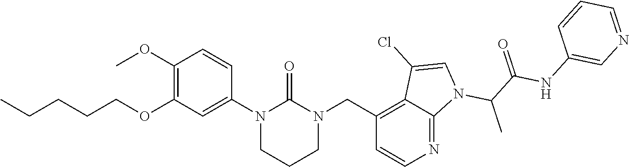 [SMILES: CCCCCOc1cc(N2CCCN(Cc3ccnc4c3c(Cl)cn4C(C)C(=O)Nc3cccnc3)C2=O)ccc1OC] |
| CU0594 | 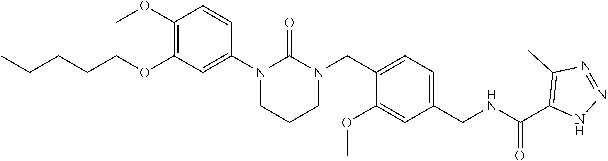 [SMILES: CCCCCOc1cc(N2CCCN(Cc3ccc(CNC(=O)c4[nH]nnc4C)cc3OC)C2=O)ccc1OC] |
| CU0595 | 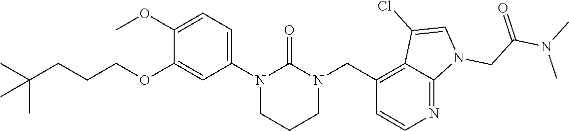 [SMILES: COc1ccc(N2CCCN(Cc3ccnc4c3c(Cl)cn4CC(=O)N(C)C)C2=O)cc1OCCCC(C)(C)C] |
| CU0596 | 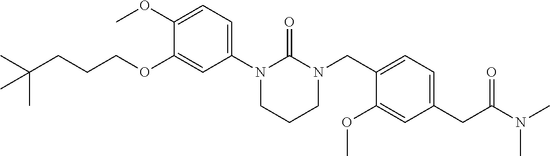 [SMILES: COc1cc(CC(=O)N(C)C)ccc1CN1CCCN(c2ccc(OC)c(OCCCC(C)(C)C)c2)C1=O] |
| CU0597 | 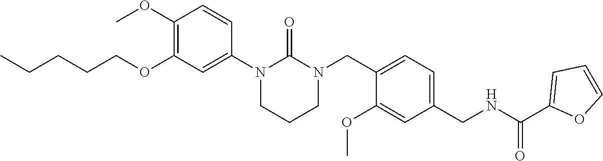 [SMILES: CCCCCOc1cc(N2CCCN(Cc3ccc(CNC(=O)c4ccco4)cc3OC)C2=O)ccc1OC] |
| CU0598 | 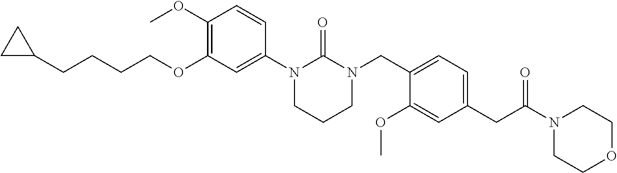 [SMILES: COc1cc(CC(=O)N2CCOCC2)ccc1CN1CCCN(c2ccc(OC)c(OCCCCC3CC3)c2)C1=O] |
| CU0599 | 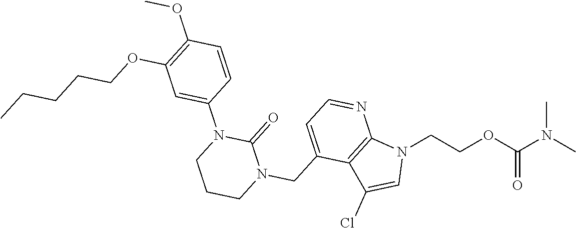 [SMILES: CCCCCOc1cc(N2CCCN(Cc3ccnc4c3c(Cl)cn4CCOC(=O)N(C)C)C2=O)ccc1OC] |
| CU0600 | 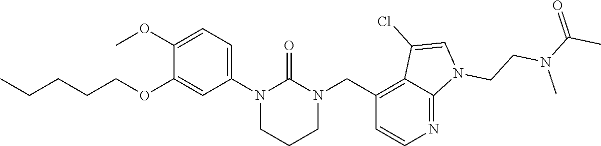 [SMILES: CCCCCOc1cc(N2CCCN(Cc3ccnc4c3c(Cl)cn4CCN(C)C(C)=O)C2=O)ccc1OC] |
| CU0601 | 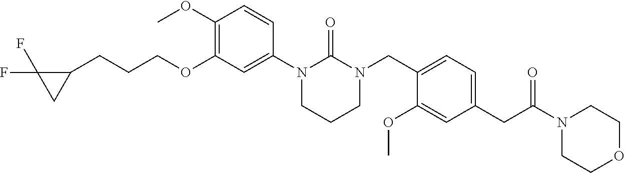 [SMILES: COc1cc(CC(=O)N2CCOCC2)ccc1CN1CCCN(c2ccc(OC)c(OCCCC3CC3(F)F)c2)C1=O] |
| CU0602 | 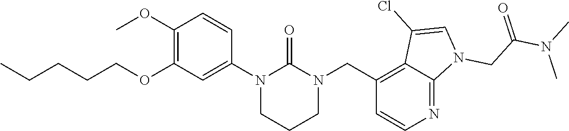 [SMILES: CCCCCOc1cc(N2CCCN(Cc3ccnc4c3c(Cl)cn4CC(=O)N(C)C)C2=O)ccc1OC] |
| CU0603 | 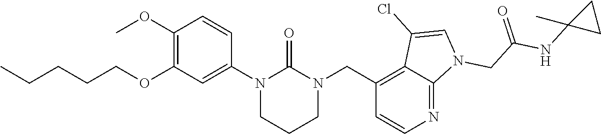 [SMILES: CCCCCOc1cc(N2CCCN(Cc3ccnc4c3c(Cl)cn4CC(=O)NC3(C)CC3)C2=O)ccc1OC] |
| CU0604 | 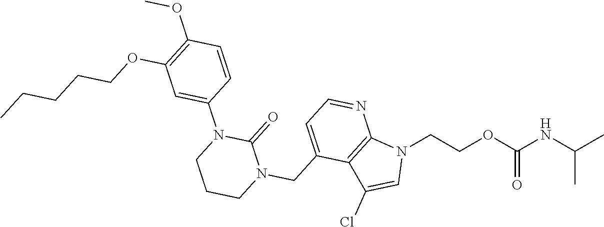 [SMILES: CCCCCOc1cc(N2CCCN(Cc3ccnc4c3c(Cl)cn4CCOC(=O)NC(C)C)C2=O)ccc1OC] |
| CU0605 | 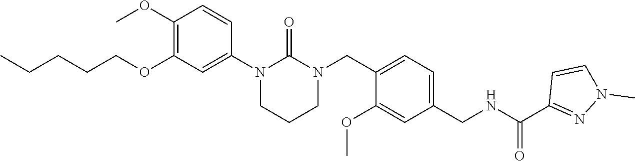 [SMILES: CCCCCOc1cc(N2CCCN(Cc3ccc(CNC(=O)c4ccn(C)n4)cc3OC)C2=O)ccc1OC] |
| CU0606 | 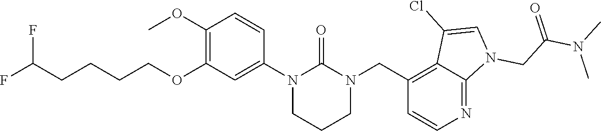 [SMILES: COc1ccc(N2CCCN(Cc3ccnc4c3c(Cl)cn4CC(=O)N(C)C)C2=O)cc1OCCCCC(F)F] |
| CU0607 | 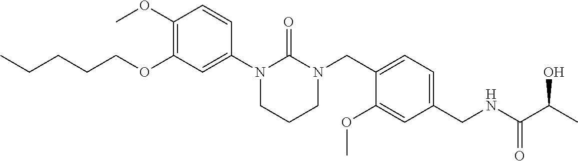 [SMILES: CCCCCOc1cc(N2CCCN(Cc3ccc(CNC(=O)[C@H](C)O)cc3OC)C2=O)ccc1OC] |
| CU0608 | 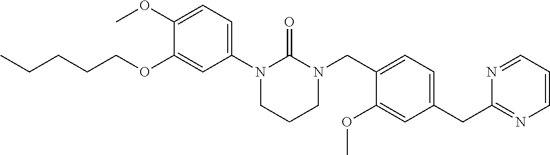 [SMILES: CCCCCOc1cc(N2CCCN(Cc3ccc(Cc4ncccn4)cc3OC)C2=O)ccc1OC] |
| CU0609 | 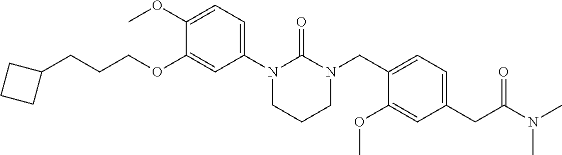 [SMILES: COc1cc(CC(=O)N(C)C)ccc1CN1CCCN(c2ccc(OC)c(OCCCC3CCC3)c2)C1=O] |
| CU0610 | 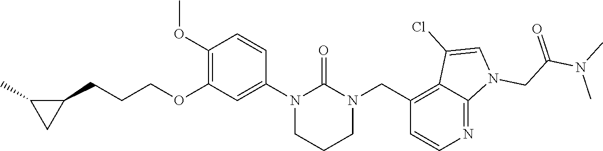 |
| CU0611 | 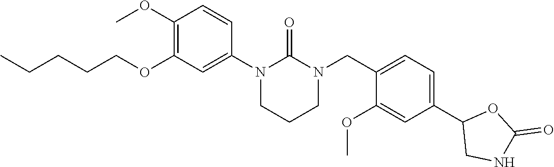 [SMILES: CCCCCOc1cc(N2CCCN(Cc3ccc(C4CNC(=O)O4)cc3OC)C2=O)ccc1OC] |
| CU0612 | 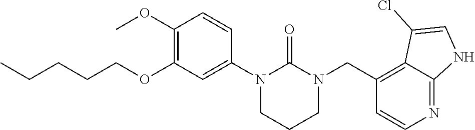 [SMILES: CCCCCOc1cc(N2CCCN(Cc3ccnc4[nH]cc(Cl)c34)C2=O)ccc1OC] |
| CU0613 | 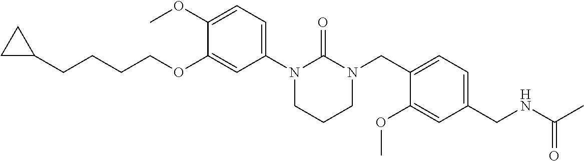 [SMILES: COc1cc(CNC(C)=O)ccc1CN1CCCN(c2ccc(OC)c(OCCCCC3CC3)c2)C1=O] |
| CU0614 | 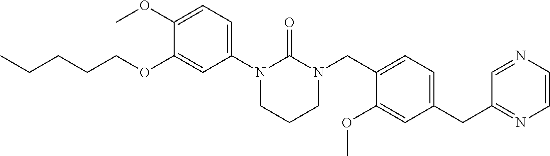 [SMILES: CCCCCOc1cc(N2CCCN(Cc3ccc(Cc4cnccn4)cc3OC)C2=O)ccc1OC] |
| CU0615 | 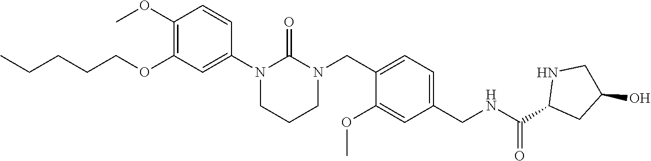 [SMILES: CCCCCOc1cc(N2CCCN(Cc3ccc(CNC(=O)[C@H]4C[C@H](O)CN4)cc3OC)C2=O)ccc1OC] |
| CU0616 | 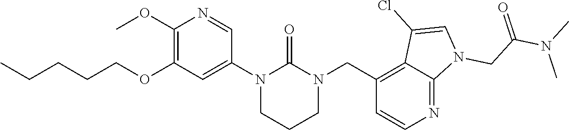 [SMILES: CCCCCOc1cc(N2CCCN(Cc3ccnc4c3c(Cl)cn4CC(=O)N(C)C)C2=O)cnc1OC] |
| CU0617 | 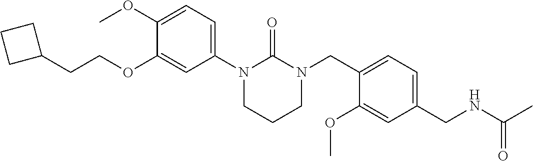 [SMILES: COc1cc(CNC(C)=O)ccc1CN1CCCN(c2ccc(OC)c(OCCC3CCC3)c2)C1=O] |
| CU0618 | 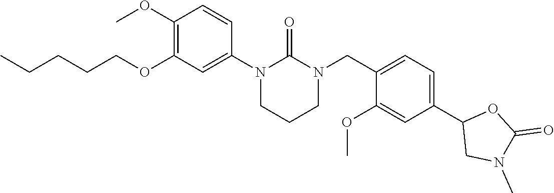 [SMILES: CCCCCOc1cc(N2CCCN(Cc3ccc(C4CN(C)C(=O)O4)cc3OC)C2=O)ccc1OC] |
| CU0619 | 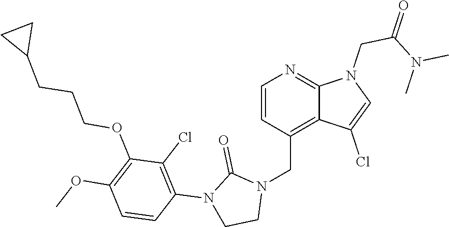 [SMILES: COc1ccc(N2CCN(Cc3ccnc4c3c(Cl)cn4CC(=O)N(C)C)C2=O)c(Cl)c1OCCCC1CC1] |
| CU0620 | 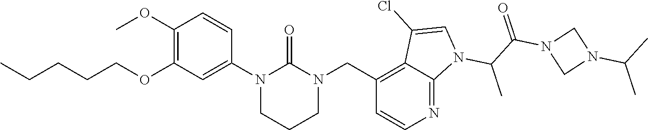 [SMILES: CCCCCOc1cc(N2CCCN(Cc3ccnc4c3c(Cl)cn4C(C)C(=O)N3CN(C(C)C)C3)C2=O)ccc1OC] |
| CU0621 | 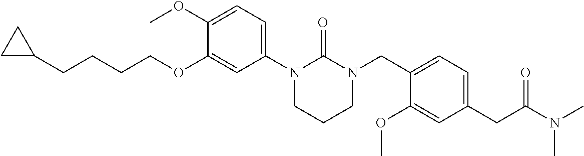 [SMILES: COc1cc(CC(=O)N(C)C)ccc1CN1CCCN(c2ccc(OC)c(OCCCCC3CC3)c2)C1=O] |
| CU0622 | 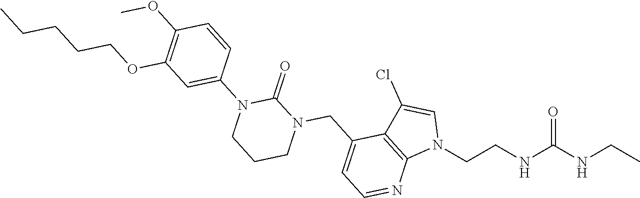 [SMILES: CCCCCOc1cc(N2CCCN(Cc3ccnc4c3c(Cl)cn4CCNC(=O)NCC)C2=O)ccc1OC] |
| CU0624 | 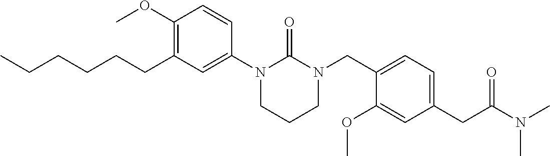 [SMILES: CCCCCCc1cc(N2CCCN(Cc3ccc(CC(=O)N(C)C)cc3OC)C2=O)ccc1OC] |
| CU0625 | 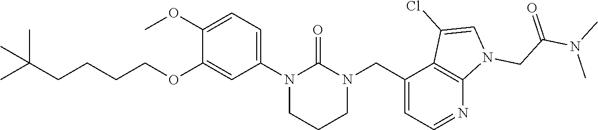 [SMILES: COc1ccc(N2CCCN(Cc3ccnc4c3c(Cl)cn4CC(=O)N(C)C)C2=O)cc1OCCCCC(C)(C)C] |
| CU0626 | 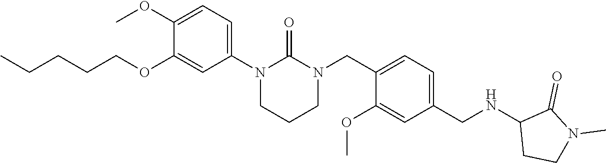 [SMILES: CCCCCOc1cc(N2CCCN(Cc3ccc(CNC4CCN(C)C4=O)cc3OC)C2=O)ccc1OC] |
| CU0627 | 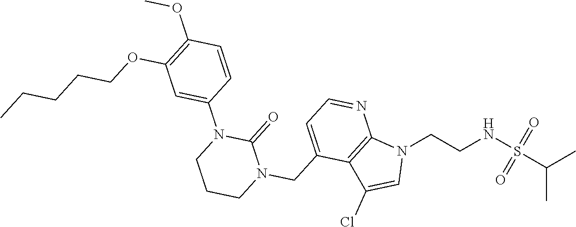 [SMILES: CCCCCOc1cc(N2CCCN(Cc3ccnc4c3c(Cl)cn4CCNS(=O)(=O)C(C)C)C2=O)ccc1OC] |
| CU0628 | 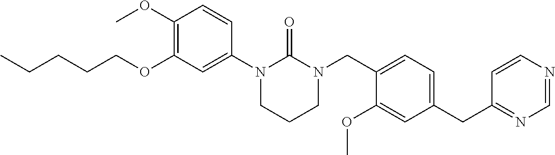 [SMILES: CCCCCOc1cc(N2CCCN(Cc3ccc(Cc4ccncn4)cc3OC)C2=O)ccc1OC] |
| CU0629 | 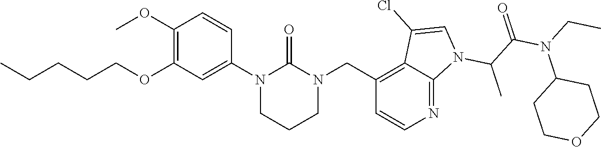 [SMILES: CCCCCOc1cc(N2CCCN(Cc3ccnc4c3c(Cl)cn4C(C)C(=O)N(CC)C3CCOCC3)C2=O)ccc1OC] |
| CU0630 | 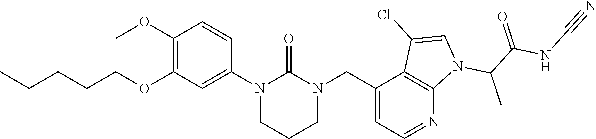 [SMILES: CCCCCOc1cc(N2CCCN(Cc3ccnc4c3c(Cl)cn4C(C)C(=O)NC#N)C2=O)ccc1OC] |
| CU0631 | 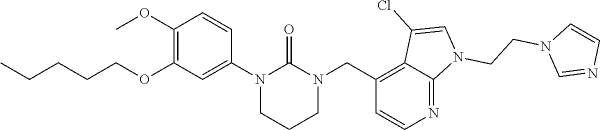 [SMILES: CCCCCOc1cc(N2CCCN(Cc3ccnc4c3c(Cl)cn4CCn3ccnc3)C2=O)ccc1OC] |
| CU0632 | 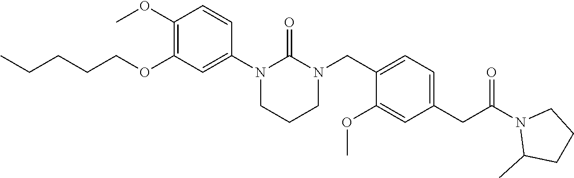 [SMILES: CCCCCOc1cc(N2CCCN(Cc3ccc(CC(=O)N4CCCC4C)cc3OC)C2=O)ccc1OC] |
| CU0633 | 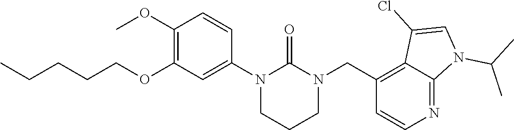 [SMILES: CCCCCOc1cc(N2CCCN(Cc3ccnc4c3c(Cl)cn4C(C)C)C2=O)ccc1OC] |
| CU0634 | 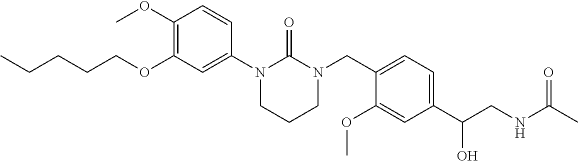 [SMILES: CCCCCOc1cc(N2CCCN(Cc3ccc(C(O)CNC(C)=O)cc3OC)C2=O)ccc1OC] |
| CU0635 | 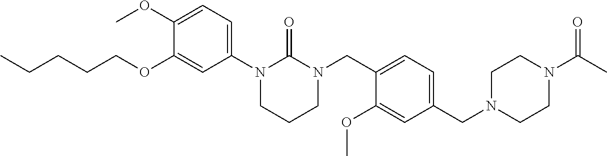 [SMILES: CCCCCOc1cc(N2CCCN(Cc3ccc(CN4CCN(C(C)=O)CC4)cc3OC)C2=O)ccc1OC] |
| CU0636 | 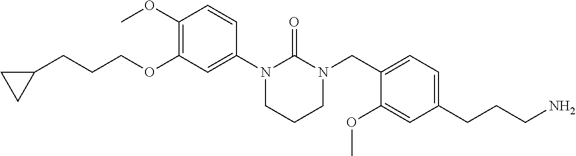 [SMILES: COc1cc(CCCN)ccc1CN1CCCN(c2ccc(OC)c(OCCCC3CC3)c2)C1=O] |
| CU0637 | 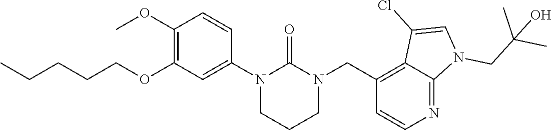 [SMILES: CCCCCOc1cc(N2CCCN(Cc3ccnc4c3c(Cl)cn4CC(C)(C)O)C2=O)ccc1OC] |
| CU0638 | 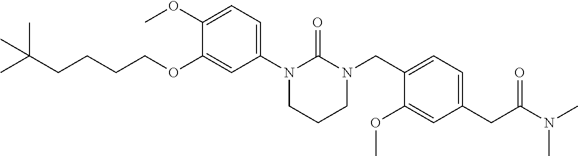 [SMILES: COc1cc(CC(=O)N(C)C)ccc1CN1CCCN(c2ccc(OC)c(OCCCCC(C)(C)C)c2)C1=O] |
| CU0639 | 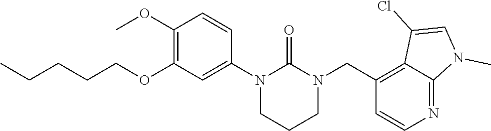 [SMILES: CCCCCOc1cc(N2CCCN(Cc3ccnc4c3c(Cl)cn4C)C2=O)ccc1OC] |
| CU0640 | 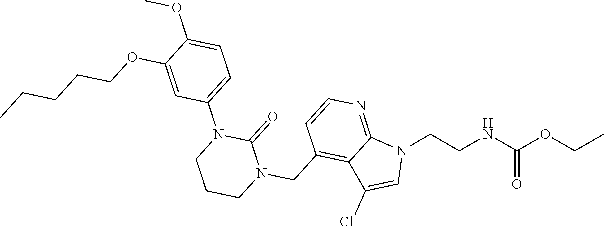 [SMILES: CCCCCOc1cc(N2CCCN(Cc3ccnc4c3c(Cl)cn4CCNC(=O)OCC)C2=O)ccc1OC] |
| CU0641 | 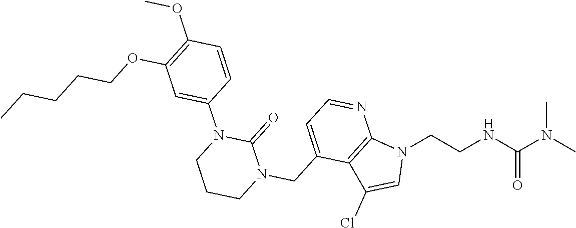 [SMILES: CCCCCOc1cc(N2CCCN(Cc3ccnc4c3c(Cl)cn4CCNC(=O)N(C)C)C2=O)ccc1OC] |
| CU0642 | 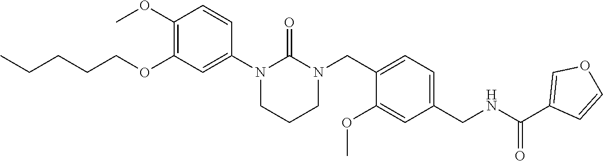 [SMILES: CCCCCOc1cc(N2CCCN(Cc3ccc(CNC(=O)c4ccoc4)cc3OC)C2=O)ccc1OC] |
| CU0643 | 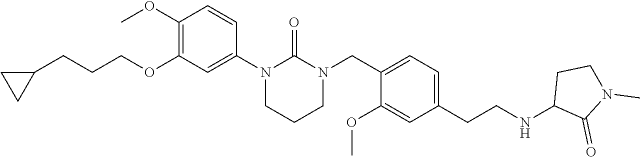 [SMILES: COc1cc(CCNC2CCN(C)C2=O)ccc1CN1CCCN(c2ccc(OC)c(OCCCC3CC3)c2)C1=O] |
| CU0644 | 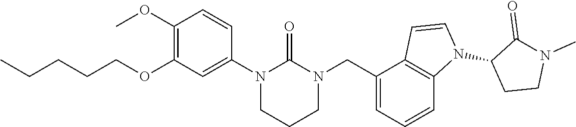 [SMILES: CCCCCOc1cc(N2CCCN(Cc3cccc4c3ccn4[C@H]3CCN(C)C3=O)C2=O)ccc1OC] |
| CU0645 | 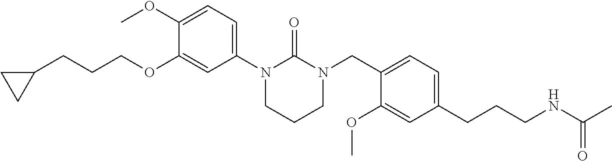 [SMILES: COc1cc(CCCNC(C)=O)ccc1CN1CCCN(c2ccc(OC)c(OCCCC3CC3)c2)C1=O] |
| CU0646 | 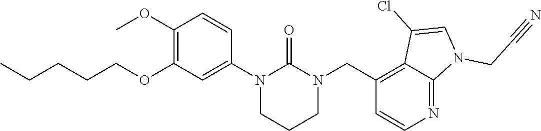 [SMILES: CCCCCOc1cc(N2CCCN(Cc3ccnc4c3c(Cl)cn4CC#N)C2=O)ccc1OC] |
| CU0647 | 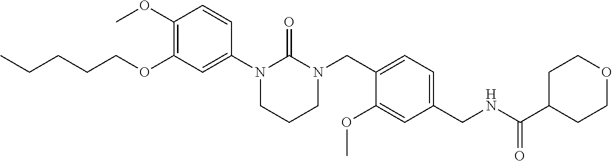 [SMILES: CCCCCOc1cc(N2CCCN(Cc3ccc(CNC(=O)C4CCOCC4)cc3OC)C2=O)ccc1OC] |
| CU0648 | 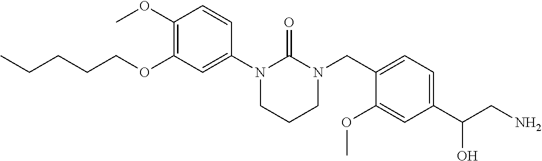 [SMILES: CCCCCOc1cc(N2CCCN(Cc3ccc(C(O)CN)cc3OC)C2=O)ccc1OC] |
| CU0649 | 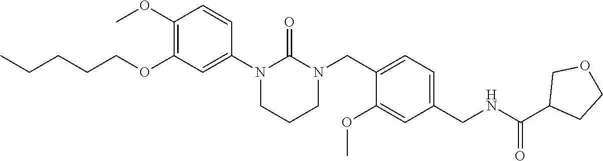 [SMILES: CCCCCOc1cc(N2CCCN(Cc3ccc(CNC(=O)C4CCOC4)cc3OC)C2=O)ccc1OC] |
| CU0650 | 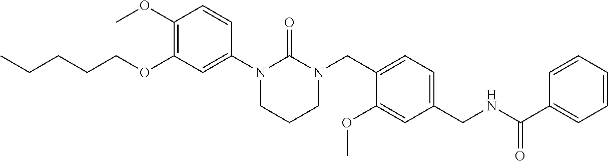 [SMILES: CCCCCOc1cc(N2CCCN(Cc3ccc(CNC(=O)c4ccccc4)cc3OC)C2=O)ccc1OC] |
| CU0651 | 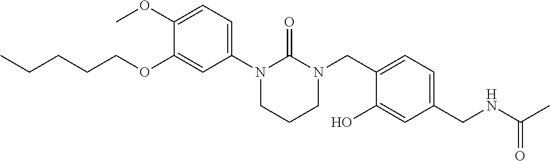 [SMILES: CCCCCOc1cc(N2CCCN(Cc3ccc(CNC(C)=O)cc3O)C2=O)ccc1OC] |
| CU0652 | 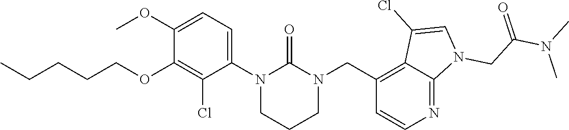 [SMILES: CCCCCOc1c(OC)ccc(N2CCCN(Cc3ccnc4c3c(Cl)cn4CC(=O)N(C)C)C2=O)c1Cl] |
| CU0653 | 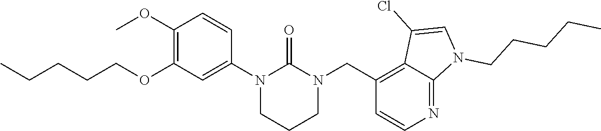 [SMILES: CCCCCOc1cc(N2CCCN(Cc3ccnc4c3c(Cl)cn4CCCCC)C2=O)ccc1OC] |
| CU0654 | 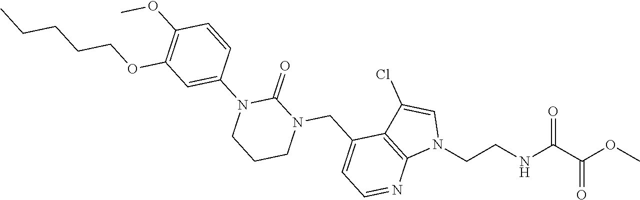 [SMILES: CCCCCOc1cc(N2CCCN(Cc3ccnc4c3c(Cl)cn4CCNC(=O)C(=O)OC)C2=O)ccc1OC] |
| CU0655 | 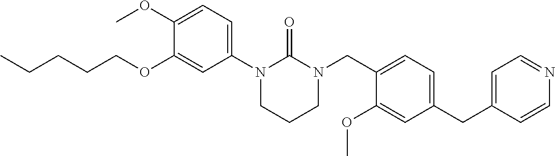 [SMILES: CCCCCOc1cc(N2CCCN(Cc3ccc(Cc4ccncc4)cc3OC)C2=O)ccc1OC] |
| CU0656 | 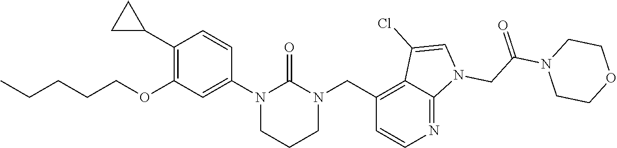 [SMILES: CCCCCOc1cc(N2CCCN(Cc3ccnc4c3c(Cl)cn4CC(=O)N3CCOCC3)C2=O)ccc1C1CC1] |
| CU0657 | 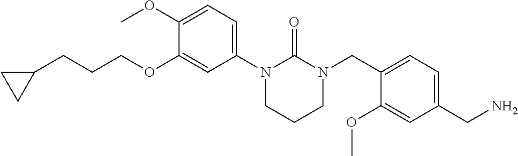 [SMILES: COc1cc(CN)ccc1CN1CCCN(c2ccc(OC)c(OCCCC3CC3)c2)C1=O] |
| CU0658 | 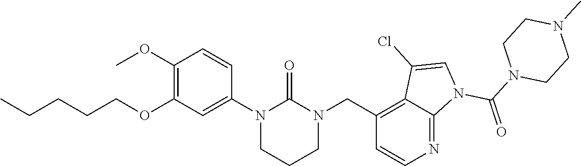 [SMILES: CCCCCOc1cc(N2CCCN(Cc3ccnc4c3c(Cl)cn4C(=O)N3CCN(C)CC3)C2=O)ccc1OC] |
| CU0659 | 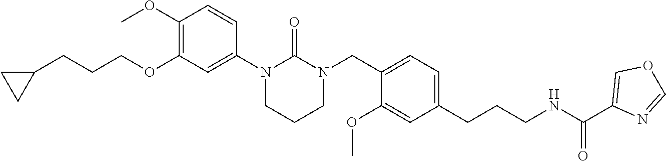 [SMILES: COc1cc(CCCNC(=O)c2cocn2)ccc1CN1CCCN(c2ccc(OC)c(OCCCC3CC3)c2)C1=O] |
| CU0660 | 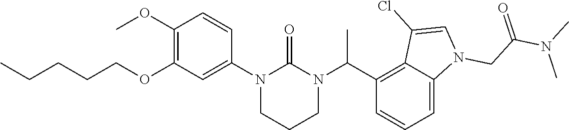 [SMILES: CCCCCOc1cc(N2CCCN(C(C)c3cccc4c3c(Cl)cn4CC(=O)N(C)C)C2=O)ccc1OC] |
| CU0661 | 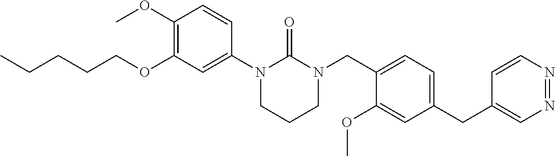 [SMILES: CCCCCOc1cc(N2CCCN(Cc3ccc(Cc4ccnnc4)cc3OC)C2=O)ccc1OC] |
| CU0662 | 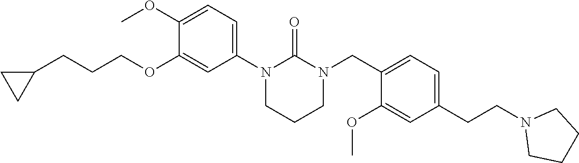 [SMILES: COc1cc(CCN2CCCC2)ccc1CN1CCCN(c2ccc(OC)c(OCCCC3CC3)c2)C1=O] |
| CU0663 | 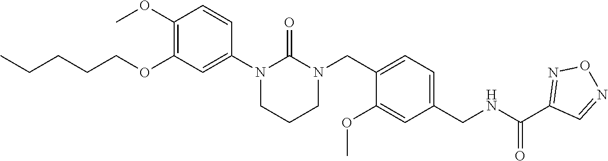 [SMILES: CCCCCOc1cc(N2CCCN(Cc3ccc(CNC(=O)c4cnon4)cc3OC)C2=O)ccc1OC] |
| CU0664 | 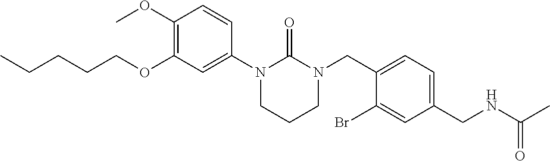 [SMILES: CCCCCOc1cc(N2CCCN(Cc3ccc(CNC(C)=O)cc3Br)C2=O)ccc1OC] |
| CU0665 | 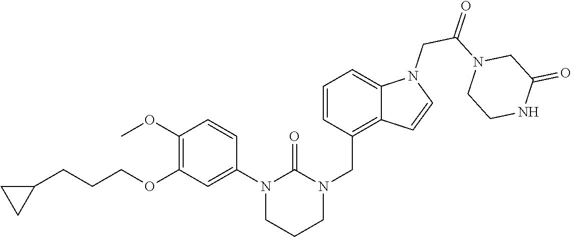 [SMILES: COc1ccc(N2CCCN(Cc3cccc4c3ccn4CC(=O)N3CCNC(=O)C3)C2=O)cc1OCCCC1CC1] |
| CU0666 | 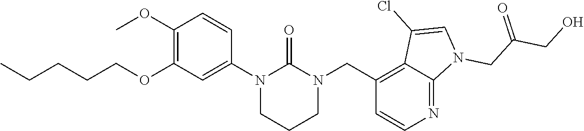 [SMILES: CCCCCOc1cc(N2CCCN(Cc3ccnc4c3c(Cl)cn4CC(=O)CO)C2=O)ccc1OC] |
| CU0667 | 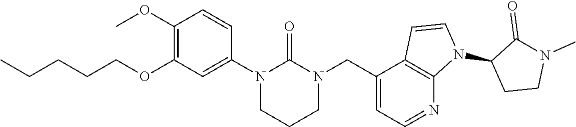 [SMILES: CCCCCOc1cc(N2CCCN(Cc3ccnc4c3ccn4[C@@H]3CCN(C)C3=O)C2=O)ccc1OC] |
| CU0668 | 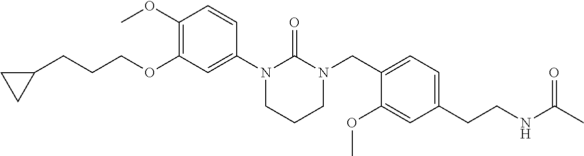 [SMILES: COc1cc(CCNC(C)=O)ccc1CN1CCCN(c2ccc(OC)c(OCCCC3CC3)c2)C1=O] |
| CU0669 | 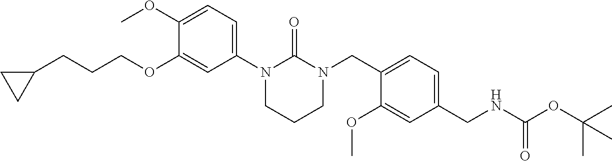 [SMILES: COc1cc(CNC(=O)OC(C)(C)C)ccc1CN1CCCN(c2ccc(OC)c(OCCCC3CC3)c2)C1=O] |
| CU0670 | 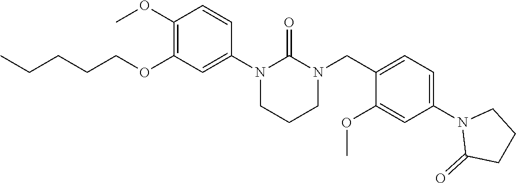 [SMILES: CCCCCOc1cc(N2CCCN(Cc3ccc(N4CCCC4=O)cc3OC)C2=O)ccc1OC] |
| CU0671 | 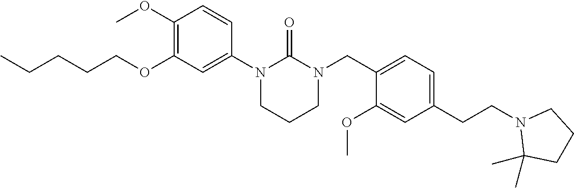 [SMILES: CCCCCOc1cc(N2CCCN(Cc3ccc(CCN4CCCC4(C)C)cc3OC)C2=O)ccc1OC] |
| CU0672 | 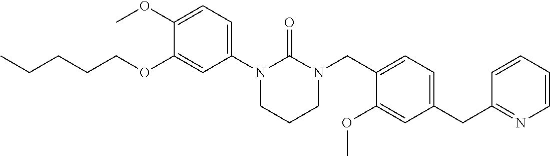 [SMILES: CCCCCOc1cc(N2CCCN(Cc3ccc(Cc4ccccn4)cc3OC)C2=O)ccc1OC] |
| CU0673 | 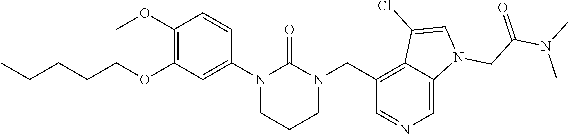 [SMILES: CCCCCOc1cc(N2CCCN(Cc3cncc4c3c(Cl)cn4CC(=O)N(C)C)C2=O)ccc1OC] |
| CU0674 | 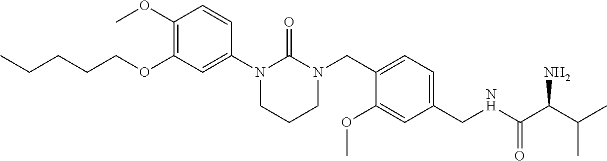 [SMILES: CCCCCOc1cc(N2CCCN(Cc3ccc(CNC(=O)[C@@H](N)C(C)C)cc3OC)C2=O)ccc1OC] |
| CU0675 | 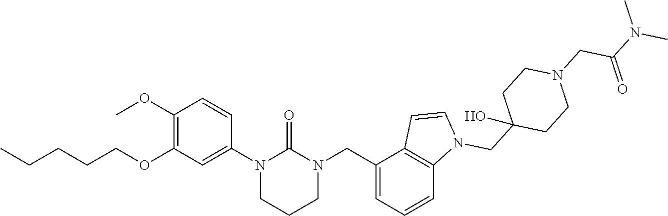 [SMILES: CCCCCOc1cc(N2CCCN(Cc3cccc4c3ccn4CC3(O)CCN(CC(=O)N(C)C)CC3)C2=O)ccc1OC] |
| CU0676 | 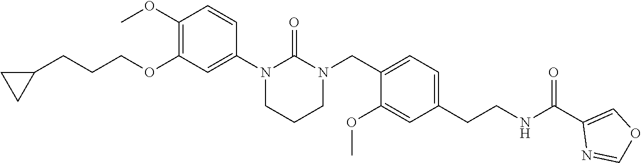 [SMILES: COc1cc(CCNC(=O)c2cocn2)ccc1CN1CCCN(c2ccc(OC)c(OCCCC3CC3)c2)C1=O] |
| CU0677 | 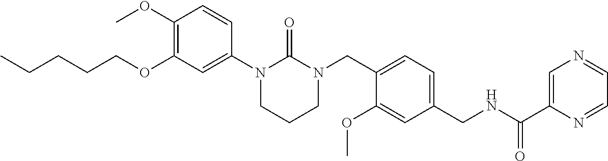 [SMILES: CCCCCOc1cc(N2CCCN(Cc3ccc(CNC(=O)c4cnccn4)cc3OC)C2=O)ccc1OC] |
| CU0678 | 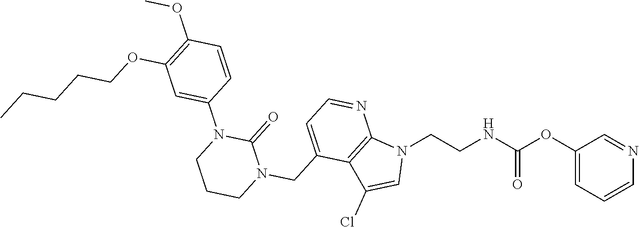 [SMILES: CCCCCOc1cc(N2CCCN(Cc3ccnc4c3c(Cl)cn4CCNC(=O)Oc3cccnc3)C2=O)ccc1OC] |
| CU0679 | 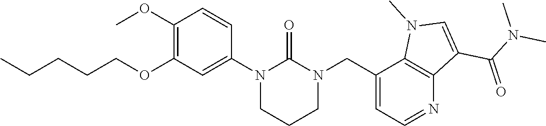 [SMILES: CCCCCOc1cc(N2CCCN(Cc3ccnc4c(C(=O)N(C)C)cn(C)c34)C2=O)ccc1OC] |
| CU0680 | 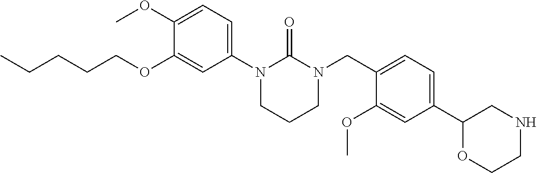 [SMILES: CCCCCOc1cc(N2CCCN(Cc3ccc(C4CNCCO4)cc3OC)C2=O)ccc1OC] |
| CU0681 | 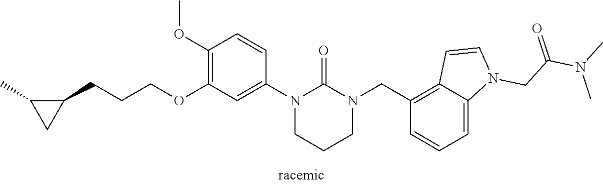 |
| CU0682 | 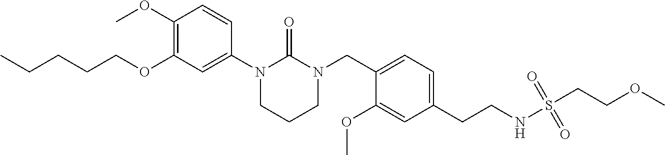 [SMILES: CCCCCOc1cc(N2CCCN(Cc3ccc(CCNS(=O)(=O)CCOC)cc3OC)C2=O)ccc1OC] |
| CU0683 | 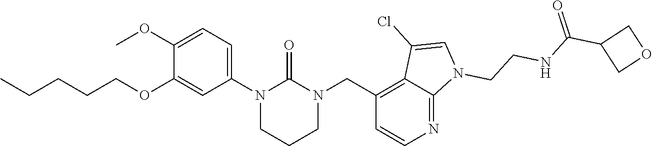 [SMILES: CCCCCOc1cc(N2CCCN(Cc3ccnc4c3c(Cl)cn4CCNC(=O)C3COC3)C2=O)ccc1OC] |
| CU0684 | 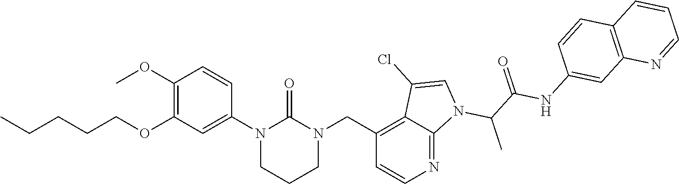 [SMILES: CCCCCOc1cc(N2CCCN(Cc3ccnc4c3c(Cl)cn4C(C)C(=O)Nc3ccc4cccnc4c3)C2=O)ccc1OC] |
| CU0685 | 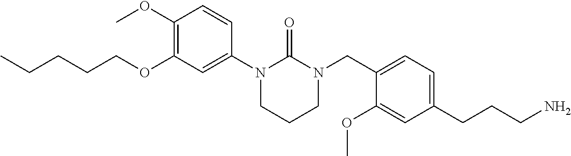 [SMILES: CCCCCOc1cc(N2CCCN(Cc3ccc(CCCN)cc3OC)C2=O)ccc1OC] |
| CU0686 | 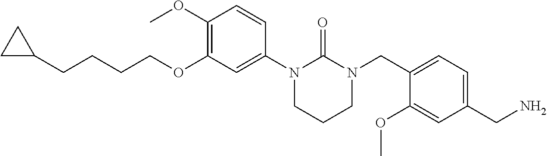 [SMILES: COc1cc(CN)ccc1CN1CCCN(c2ccc(OC)c(OCCCCC3CC3)c2)C1=O] |
| CU0687 | 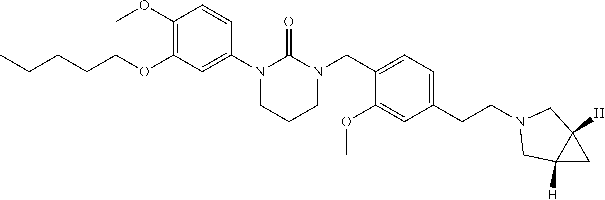 [SMILES: CCCCCOc1cc(N2CCCN(Cc3ccc(CCN4C[C@H]5C[C@H]5C4)cc3OC)C2=O)ccc1OC] |
| CU0688 | 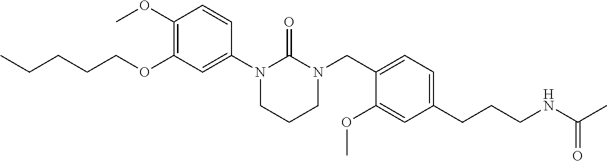 [SMILES: CCCCCOc1cc(N2CCCN(Cc3ccc(CCCNC(C)=O)cc3OC)C2=O)ccc1OC] |
| CU0689 | 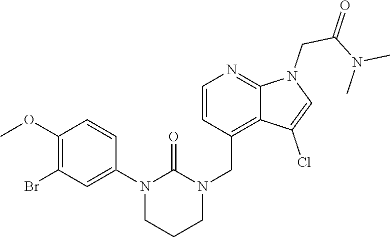 |
| CU0690 | 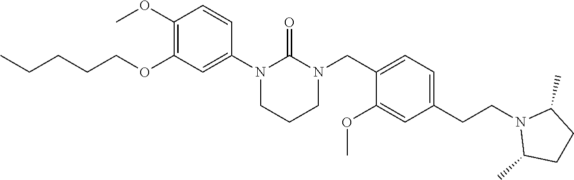 [SMILES: CCCCCOc1cc(N2CCCN(Cc3ccc(CCN4[C@H](C)CC[C@@H]4C)cc3OC)C2=O)ccc1OC] |
| CU0691 | 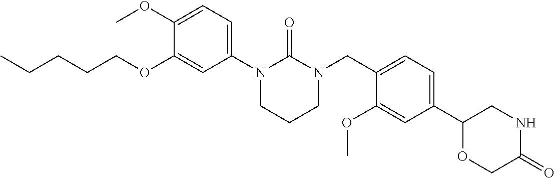 [SMILES: CCCCCOc1cc(N2CCCN(Cc3ccc(C4CNC(=O)CO4)cc3OC)C2=O)ccc1OC] |
| CU0692 | 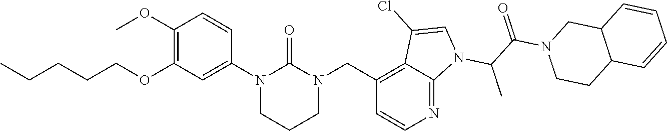 [SMILES: CCCCCOc1cc(N2CCCN(Cc3ccnc4c3c(Cl)cn4C(C)C(=O)N3CCC4C=CC=CC4C3)C2=O)ccc1OC] |
| CU0693 | 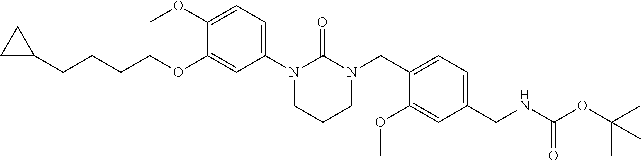 [SMILES: COc1cc(CNC(=O)OC(C)(C)C)ccc1CN1CCCN(c2ccc(OC)c(OCCCCC3CC3)c2)C1=O] |
| CU0694 | 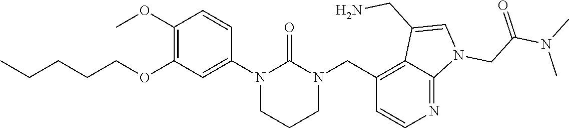 [SMILES: CCCCCOc1cc(N2CCCN(Cc3ccnc4c3c(CN)cn4CC(=O)N(C)C)C2=O)ccc1OC] |
| CU0695 | 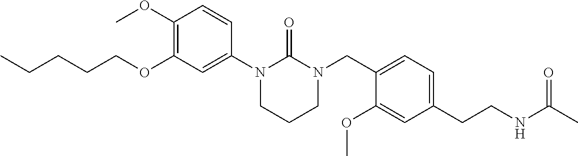 [SMILES: CCCCCOc1cc(N2CCCN(Cc3ccc(CCNC(C)=O)cc3OC)C2=O)ccc1OC] |
| CU0696 | 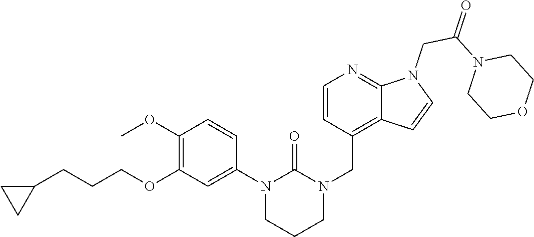 [SMILES: COc1ccc(N2CCCN(Cc3ccnc4c3ccn4CC(=O)N3CCOCC3)C2=O)cc1OCCCC1CC1] |
| CU0697 | 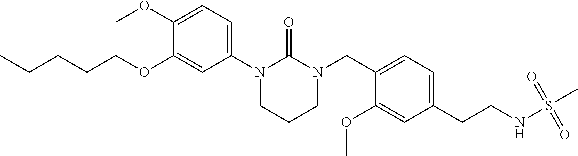 [SMILES: CCCCCOc1cc(N2CCCN(Cc3ccc(CCNS(C)(=O)=O)cc3OC)C2=O)ccc1OC] |
| CU0698 | 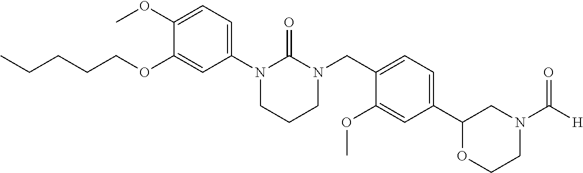 [SMILES: CCCCCOc1cc(N2CCCN(Cc3ccc(C4CN(C=O)CCO4)cc3OC)C2=O)ccc1OC] |
| CU0699 | 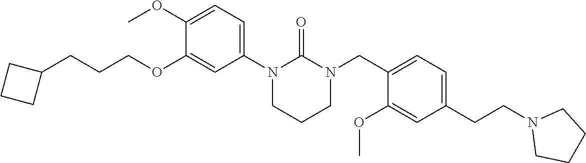 [SMILES: COc1cc(CCN2CCCC2)ccc1CN1CCCN(c2ccc(OC)c(OCCCC3CCC3)c2)C1=O] |
| CU0700 | 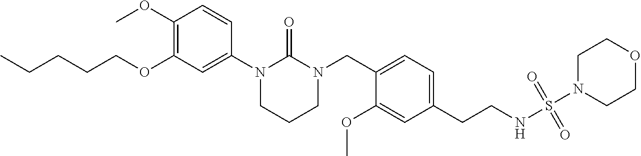 [SMILES: CCCCCOc1cc(N2CCCN(Cc3ccc(CCNS(=O)(=O)N4CCOCC4)cc3OC)C2=O)ccc1OC] |
| CU0701 | 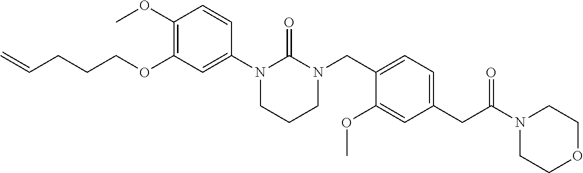 [SMILES: C=CCCCOc1cc(N2CCCN(Cc3ccc(CC(=O)N4CCOCC4)cc3OC)C2=O)ccc1OC] |
| CU0702 | 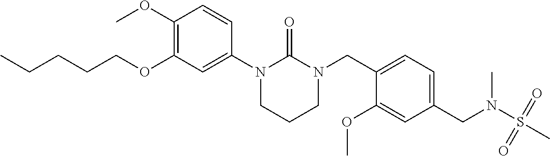 [SMILES: CCCCCOc1cc(N2CCCN(Cc3ccc(CN(C)S(C)(=O)=O)cc3OC)C2=O)ccc1OC] |
| CU0703 | 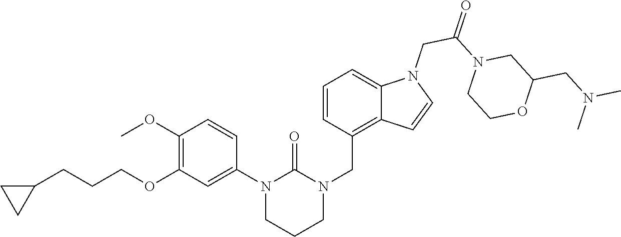 [SMILES: COc1ccc(N2CCCN(Cc3cccc4c3ccn4CC(=O)N3CCOC(CN(C)C)C3)C2=O)cc1OCCCC1CC1] |
| CU0704 | 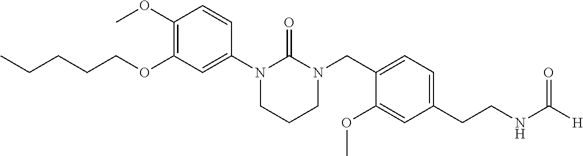 [SMILES: CCCCCOc1cc(N2CCCN(Cc3ccc(CCNC=O)cc3OC)C2=O)ccc1OC] |
| CU0705 | 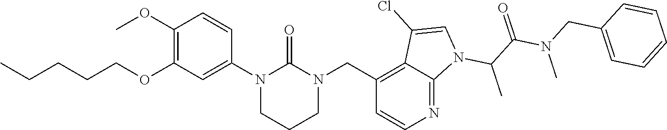 [SMILES: CCCCCOc1cc(N2CCCN(Cc3ccnc4c3c(Cl)cn4C(C)C(=O)N(C)Cc3ccccc3)C2=O)ccc1OC] |
| CU0706 | 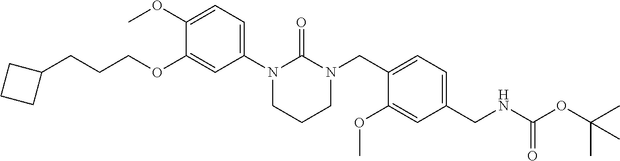 [SMILES: COc1cc(CNC(=O)OC(C)(C)C)ccc1CN1CCCN(c2ccc(OC)c(OCCCC3CCC3)c2)C1=O] |
| CU0707 | 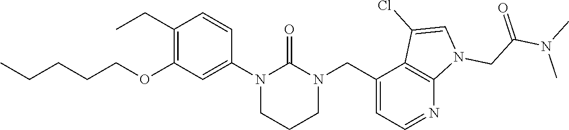 [SMILES: CCCCCOc1cc(N2CCCN(Cc3ccnc4c3c(Cl)cn4CC(=O)N(C)C)C2=O)ccc1CC] |
| CU0708 | 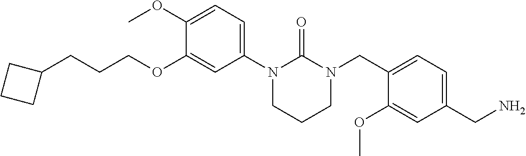 [SMILES: COc1cc(CN)ccc1CN1CCCN(c2ccc(OC)c(OCCCC3CCC3)c2)C1=O] |
| CU0709 | 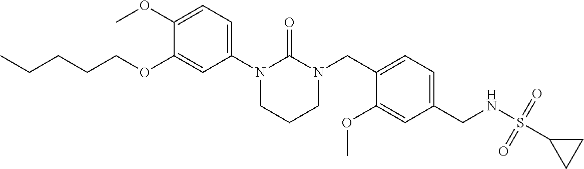 [SMILES: CCCCCOc1cc(N2CCCN(Cc3ccc(CNS(=O)(=O)C4CC4)cc3OC)C2=O)ccc1OC] |
| CU0710 | 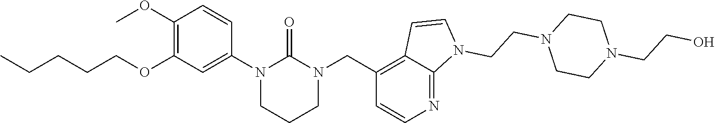 [SMILES: CCCCCOc1cc(N2CCCN(Cc3ccnc4c3ccn4CCN3CCN(CCO)CC3)C2=O)ccc1OC] |
| CU0711 | 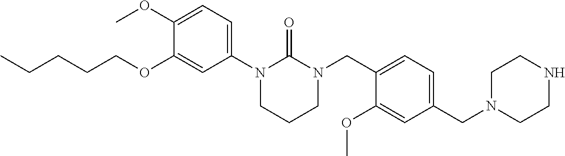 [SMILES: CCCCCOc1cc(N2CCCN(Cc3ccc(CN4CCNCC4)cc3OC)C2=O)ccc1OC] |
| CU0712 | 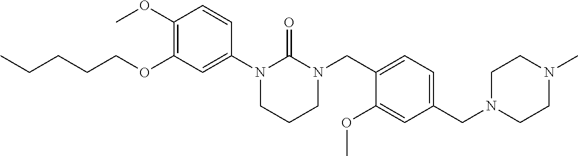 [SMILES: CCCCCOc1cc(N2CCCN(Cc3ccc(CN4CCN(C)CC4)cc3OC)C2=O)ccc1OC] |
| CU0713 | 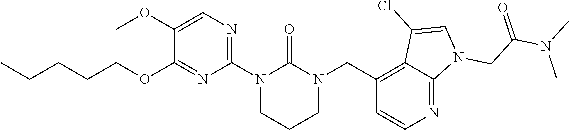 [SMILES: CCCCCOc1nc(N2CCCN(Cc3ccnc4c3c(Cl)cn4CC(=O)N(C)C)C2=O)ncc1OC] |
| CU0714 | 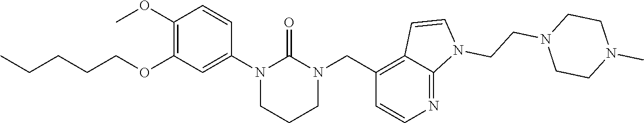 [SMILES: CCCCCOc1cc(N2CCCN(Cc3ccnc4c3ccn4CCN3CCN(C)CC3)C2=O)ccc1OC] |
| CU0715 | 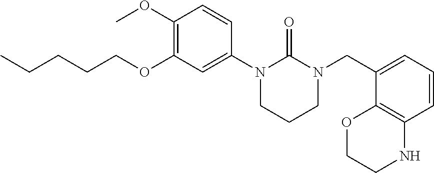 [SMILES: CCCCCOc1cc(N2CCCN(Cc3cccc4c3OCCN4)C2=O)ccc1OC] |
| CU0716 | 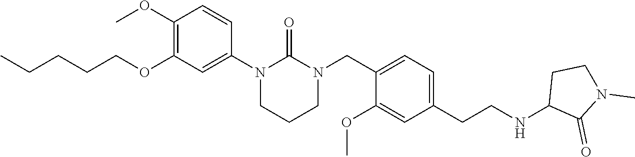 [SMILES: CCCCCOc1cc(N2CCCN(Cc3ccc(CCNC4CCN(C)C4=O)cc3OC)C2=O)ccc1OC] |
| CU0717 | 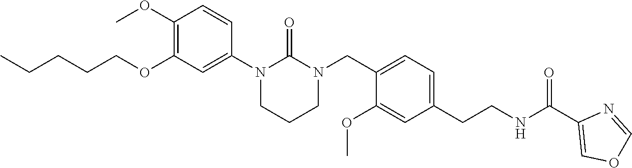 [SMILES: CCCCCOc1cc(N2CCCN(Cc3ccc(CCNC(=O)c4cocn4)cc3OC)C2=O)ccc1OC] |
| CU0718 | 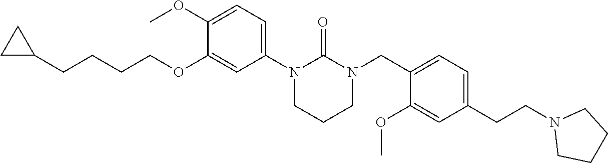 [SMILES: COc1cc(CCN2CCCC2)ccc1CN1CCCN(c2ccc(OC)c(OCCCCC3CC3)c2)C1=O] |
| CU0719 | 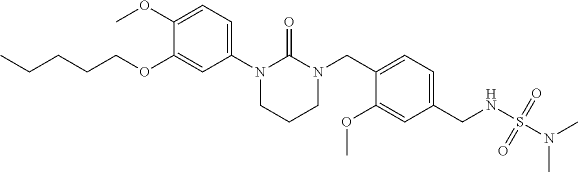 [SMILES: CCCCCOc1cc(N2CCCN(Cc3ccc(CNS(=O)(=O)N(C)C)cc3OC)C2=O)ccc1OC] |
| CU0720 | 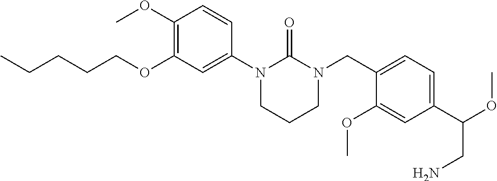 [SMILES: CCCCCOc1cc(N2CCCN(Cc3ccc(C(CN)OC)cc3OC)C2=O)ccc1OC] |
| CU0721 | 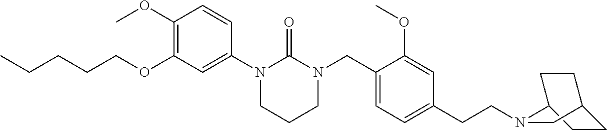 [SMILES: CCCCCOc1cc(N2CCCN(Cc3ccc(CCN4CC5CCC4CC5)cc3OC)C2=O)ccc1OC] |
| CU0722 | 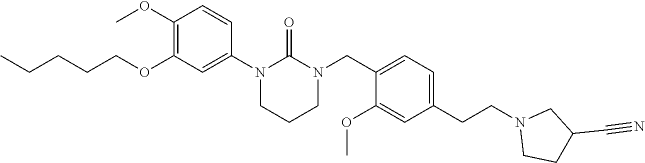 [SMILES: CCCCCOc1cc(N2CCCN(Cc3ccc(CCN4CCC(C#N)C4)cc3OC)C2=O)ccc1OC] |
| CU0723 | 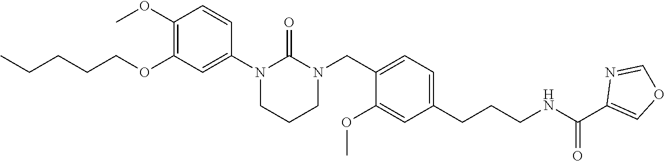 [SMILES: CCCCCOc1cc(N2CCCN(Cc3ccc(CCCNC(=O)c4cocn4)cc3OC)C2=O)ccc1OC] |
| CU0724 | 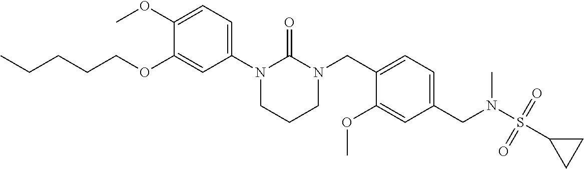 [SMILES: CCCCCOc1cc(N2CCCN(Cc3ccc(CN(C)S(=O)(=O)C4CC4)cc3OC)C2=O)ccc1OC] |
| CU0725 | 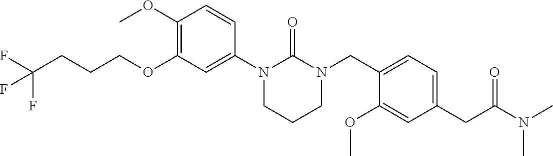 [SMILES: COc1cc(CC(=O)N(C)C)ccc1CN1CCCN(c2ccc(OC)c(OCCCC(F)(F)F)c2)C1=O] |
| CU0726 | 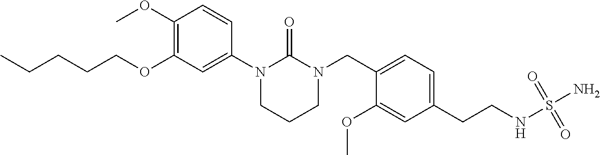 [SMILES: CCCCCOc1cc(N2CCCN(Cc3ccc(CCNS(N)(=O)=O)cc3OC)C2=O)ccc1OC] |
| CU0727 | 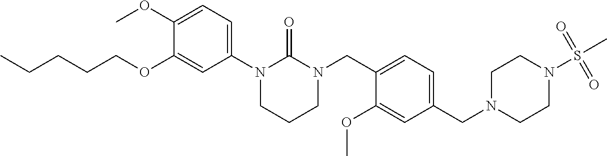 [SMILES: CCCCCOc1cc(N2CCCN(Cc3ccc(CN4CCN(S(C)(=O)=O)CC4)cc3OC)C2=O)ccc1OC] |
| CU0728 | 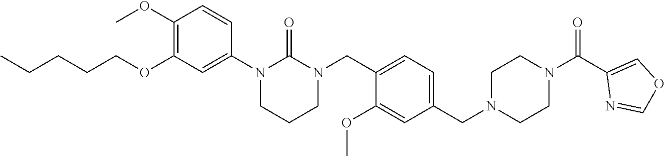 [SMILES: CCCCCOc1cc(N2CCCN(Cc3ccc(CN4CCN(C(=O)c5cocn5)CC4)cc3OC)C2=O)ccc1OC] |
| CU0729 | 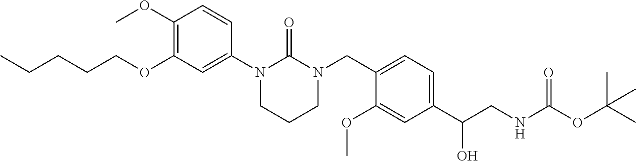 [SMILES: CCCCCOc1cc(N2CCCN(Cc3ccc(C(O)CNC(=O)OC(C)(C)C)cc3OC)C2=O)ccc1OC] |
| CU0730 | 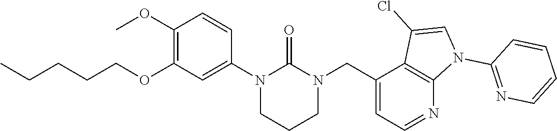 [SMILES: CCCCCOc1cc(N2CCCN(Cc3ccnc4c3c(Cl)cn4-c3ccccn3)C2=O)ccc1OC] |
| CU0731 | 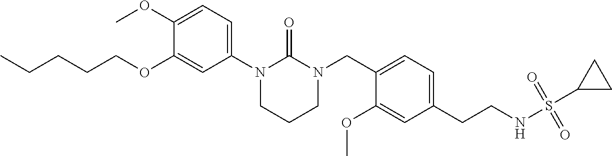 [SMILES: CCCCCOc1cc(N2CCCN(Cc3ccc(CCNS(=O)(=O)C4CC4)cc3OC)C2=O)ccc1OC] |
| CU0732 | 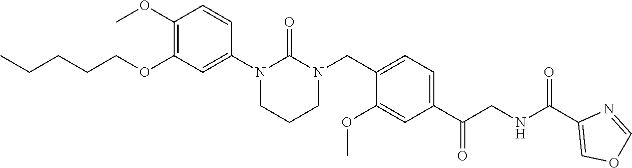 [SMILES: CCCCCOc1cc(N2CCCN(Cc3ccc(C(=O)CNC(=O)c4cocn4)cc3OC)C2=O)ccc1OC] |
| CU0733 | 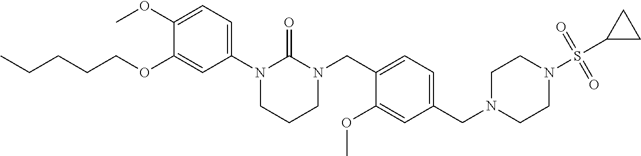 [SMILES: CCCCCOc1cc(N2CCCN(Cc3ccc(CN4CCN(S(=O)(=O)C5CC5)CC4)cc3OC)C2=O)ccc1OC] |
| CU0734 | 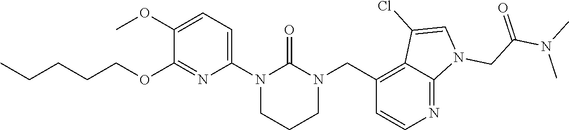 [SMILES: CCCCCOc1nc(N2CCCN(Cc3ccnc4c3c(Cl)cn4CC(=O)N(C)C)C2=O)ccc1OC] |
| CU0735 | 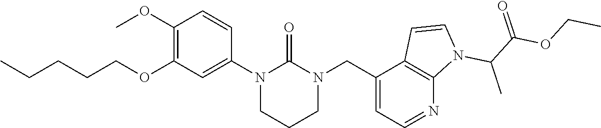 [SMILES: CCCCCOc1cc(N2CCCN(Cc3ccnc4c3ccn4C(C)C(=O)OCC)C2=O)ccc1OC] |
| CU0736 | 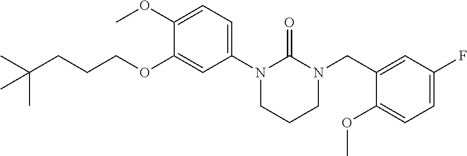 [SMILES: COc1ccc(F)cc1CN1CCCN(c2ccc(OC)c(OCCCC(C)(C)C)c2)C1=O] |
| CU0737 | 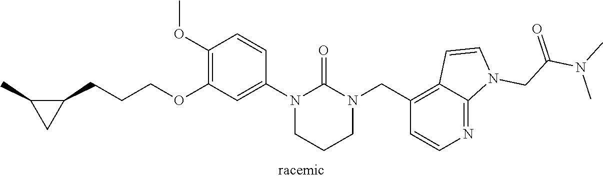 |
| CU0738 | 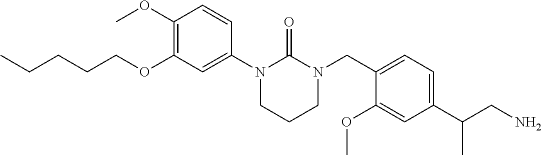 [SMILES: CCCCCOc1cc(N2CCCN(Cc3ccc(C(C)CN)cc3OC)C2=O)ccc1OC] |
| CU0739 | 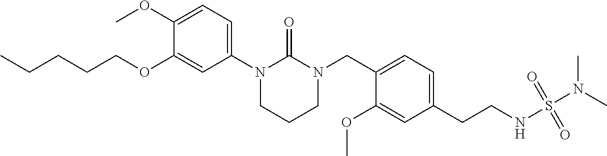 [SMILES: CCCCCOc1cc(N2CCCN(Cc3ccc(CCNS(=O)(=O)N(C)C)cc3OC)C2=O)ccc1OC] |
| CU0740 | 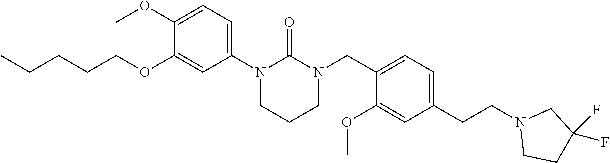 [SMILES: CCCCCOc1cc(N2CCCN(Cc3ccc(CCN4CCC(F)(F)C4)cc3OC)C2=O)ccc1OC] |
| CU0741 | 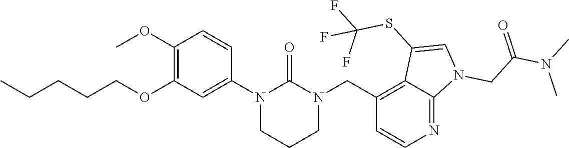 [SMILES: CCCCCOc1cc(N2CCCN(Cc3ccnc4c3c(SC(F)(F)F)cn4CC(=O)N(C)C)C2=O)ccc1OC] |
| CU0742 | 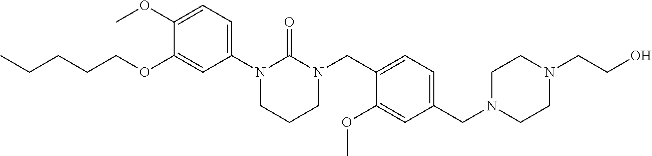 [SMILES: CCCCCOc1cc(N2CCCN(Cc3ccc(CN4CCN(CCO)CC4)cc3OC)C2=O)ccc1OC] |
| CU0743 | 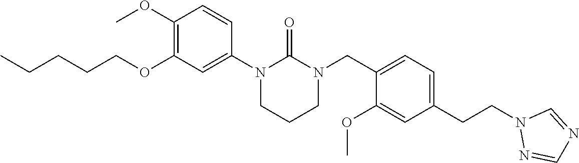 [SMILES: CCCCCOc1cc(N2CCCN(Cc3ccc(CCn4cncn4)cc3OC)C2=O)ccc1OC] |
| CU0744 | 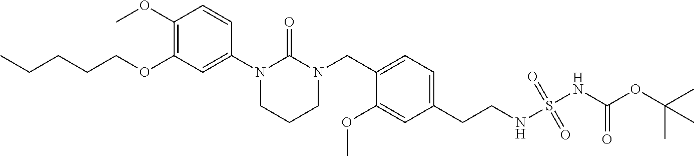 [SMILES: CCCCCOc1cc(N2CCCN(Cc3ccc(CCNS(=O)(=O)NC(=O)OC(C)(C)C)cc3OC)C2=O)ccc1OC] |
| CU0745 | 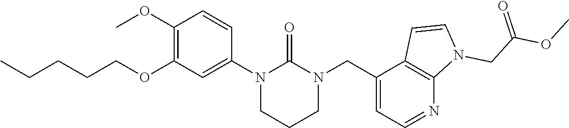 [SMILES: CCCCCOc1cc(N2CCCN(Cc3ccnc4c3ccn4CC(=O)OC)C2=O)ccc1OC] |
| CU0746 | 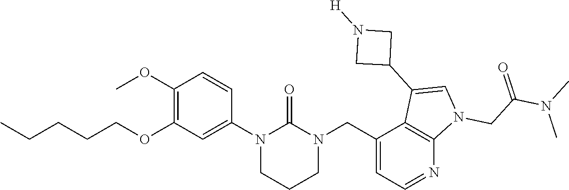 [SMILES: CCCCCOc1cc(N2CCCN(Cc3ccnc4c3c(C3CNC3)cn4CC(=O)N(C)C)C2=O)ccc1OC] |
| CU0747 | 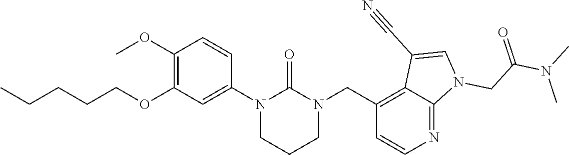 [SMILES: CCCCCOc1cc(N2CCCN(Cc3ccnc4c3c(C#N)cn4CC(=O)N(C)C)C2=O)ccc1OC] |
| CU0748 | 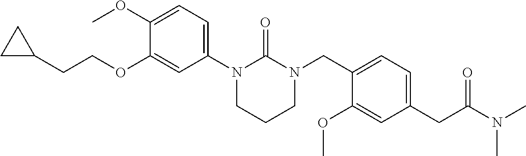 [SMILES: COc1cc(CC(=O)N(C)C)ccc1CN1CCCN(c2ccc(OC)c(OCCC3CC3)c2)C1=O] |
| CU0749 | 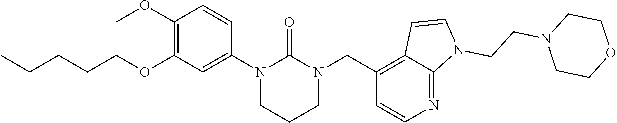 [SMILES: CCCCCOc1cc(N2CCCN(Cc3ccnc4c3ccn4CCN3CCOCC3)C2=O)ccc1OC] |
| CU0750 | 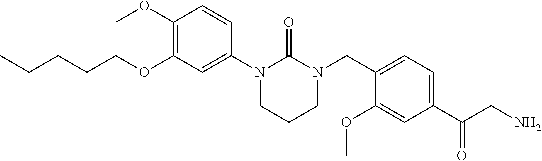 [SMILES: CCCCCOc1cc(N2CCCN(Cc3ccc(C(=O)CN)cc3OC)C2=O)ccc1OC] |
| CU0751 | 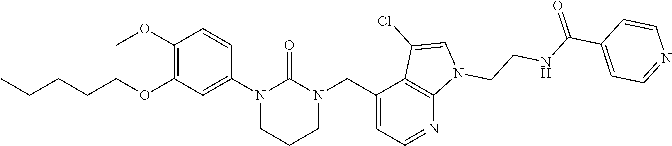 [SMILES: CCCCCOc1cc(N2CCCN(Cc3ccnc4c3c(Cl)cn4CCNC(=O)c3ccncc3)C2=O)ccc1OC] |
| CU0752 | 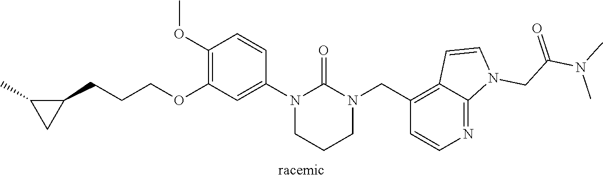 |
| CU0753 | 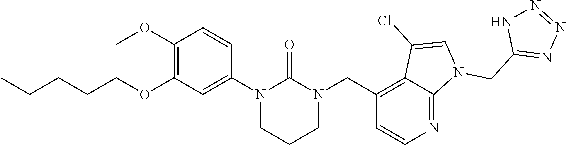 [SMILES: CCCCCOc1cc(N2CCCN(Cc3ccnc4c3c(Cl)cn4Cc3nnn[nH]3)C2=O)ccc1OC] |
| CU0754 | 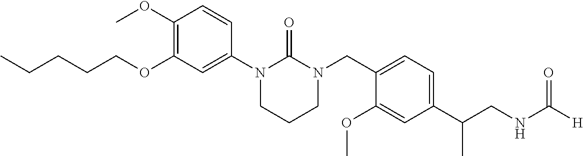 [SMILES: CCCCCOc1cc(N2CCCN(Cc3ccc(C(C)CNC=O)cc3OC)C2=O)ccc1OC] |
| CU0755 | 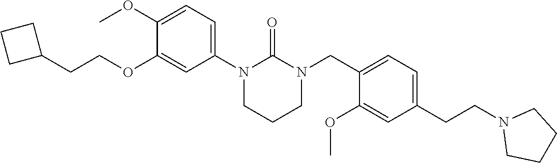 [SMILES: COc1cc(CCN2CCCC2)ccc1CN1CCCN(c2ccc(OC)c(OCCC3CCC3)c2)C1=O] |
| CU0756 | 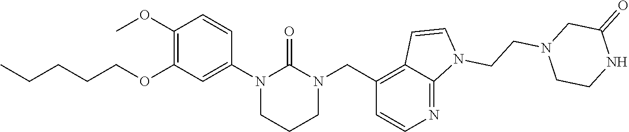 [SMILES: CCCCCOc1cc(N2CCCN(Cc3ccnc4c3ccn4CCN3CCNC(=O)C3)C2=O)ccc1OC] |
| CU0757 | 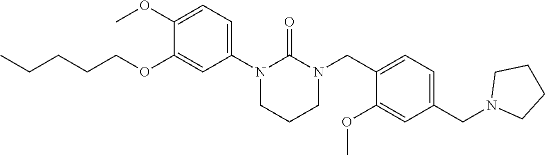 [SMILES: CCCCCOc1cc(N2CCCN(Cc3ccc(CN4CCCC4)cc3OC)C2=O)ccc1OC] |
| CU0758 | 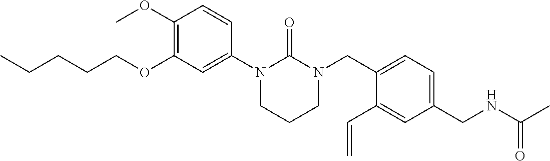 [SMILES: C=Cc1cc(CNC(C)=O)ccc1CN1CCCN(c2ccc(OC)c(OCCCCC)c2)C1=O] |
| CU0759 | 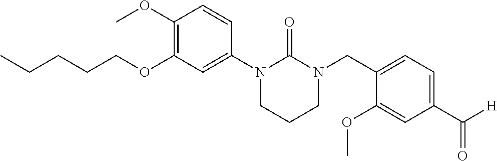 [SMILES: CCCCCOc1cc(N2CCCN(Cc3ccc(C=O)cc3OC)C2=O)ccc1OC] |
| CU0760 | 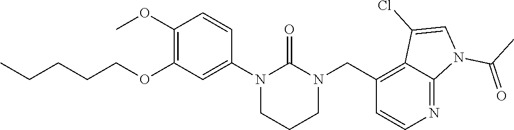 [SMILES: CCCCCOc1cc(N2CCCN(Cc3ccnc4c3c(Cl)cn4C(C)=O)C2=O)ccc1OC] |
| CU0761 | 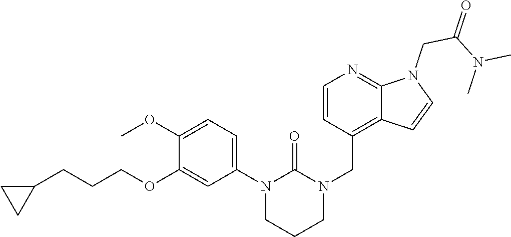 [SMILES: COc1ccc(N2CCCN(Cc3ccnc4c3ccn4CC(=O)N(C)C)C2=O)cc1OCCCC1CC1] |
| CU0762 | 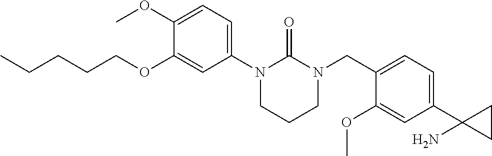 [SMILES: CCCCCOc1cc(N2CCCN(Cc3ccc(C4(N)CC4)cc3OC)C2=O)ccc1OC] |
| CU0763 | 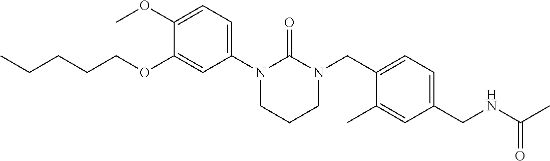 [SMILES: CCCCCOc1cc(N2CCCN(Cc3ccc(CNC(C)=O)cc3C)C2=O)ccc1OC] |
| CU0764 | 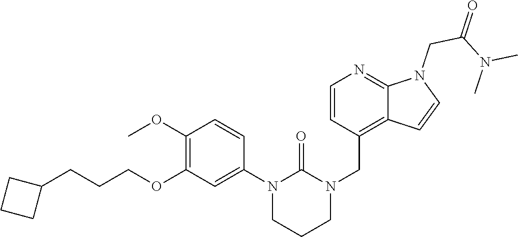 [SMILES: COc1ccc(N2CCCN(Cc3ccnc4c3ccn4CC(=O)N(C)C)C2=O)cc1OCCCC1CCC1] |
| CU0765 | 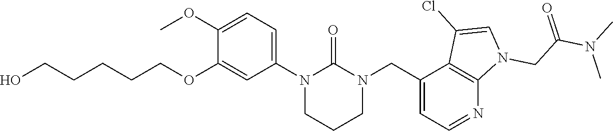 [SMILES: COc1ccc(N2CCCN(Cc3ccnc4c3c(Cl)cn4CC(=O)N(C)C)C2=O)cc1OCCCCCO] |
| CU0766 | 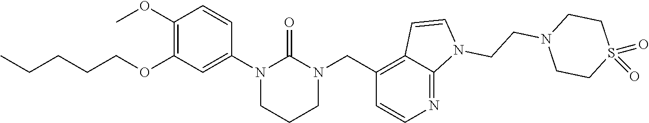 [SMILES: CCCCCOc1cc(N2CCCN(Cc3ccnc4c3ccn4CCN3CCS(=O)(=O)CC3)C2=O)ccc1OC] |
| CU0767 | 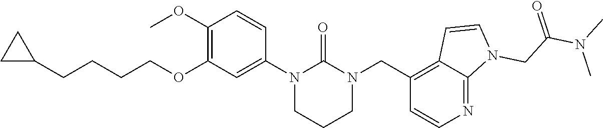 [SMILES: COc1ccc(N2CCCN(Cc3ccnc4c3ccn4CC(=O)N(C)C)C2=O)cc1OCCCCC1CC1] |
| CU0768 | 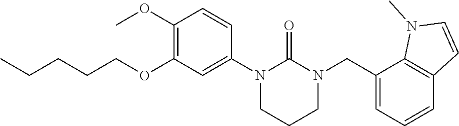 [SMILES: CCCCCOc1cc(N2CCCN(Cc3cccc4ccn(C)c34)C2=O)ccc1OC] |
| CU0769 | 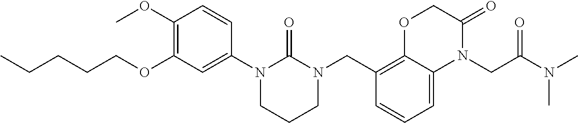 [SMILES: CCCCCOc1cc(N2CCCN(Cc3cccc4c3OCC(=O)N4CC(=O)N(C)C)C2=O)ccc1OC] |
| CU0770 | 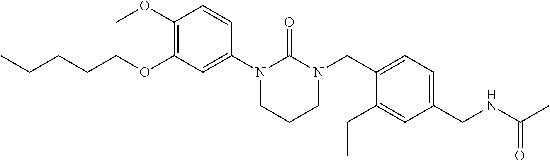 [SMILES: CCCCCOc1cc(N2CCCN(Cc3ccc(CNC(C)=O)cc3CC)C2=O)ccc1OC] |
| CU0771 | 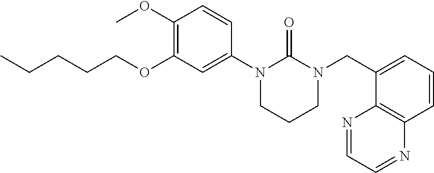 [SMILES: CCCCCOc1cc(N2CCCN(Cc3cccc4nccnc34)C2=O)ccc1OC] |
| CU0772 | 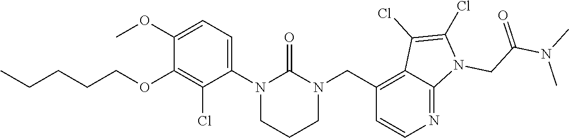 [SMILES: CCCCCOc1c(OC)ccc(N2CCCN(Cc3ccnc4c3c(Cl)c(Cl)n4CC(=O)N(C)C)C2=O)c1Cl] |
| CU0773 | 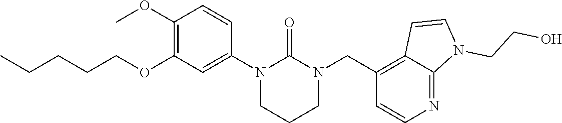 [SMILES: CCCCCOc1cc(N2CCCN(Cc3ccnc4c3ccn4CCO)C2=O)ccc1OC] |
| CU0774 | 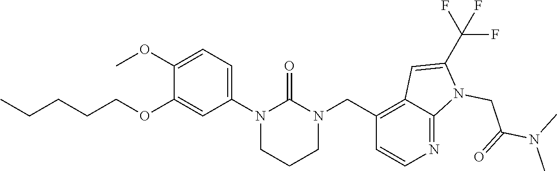 [SMILES: CCCCCOc1cc(N2CCCN(Cc3ccnc4c3cc(C(F)(F)F)n4CC(=O)N(C)C)C2=O)ccc1OC] |
| CU0775 | 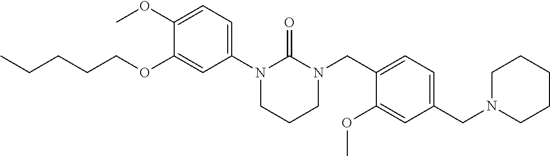 [SMILES: CCCCCOc1cc(N2CCCN(Cc3ccc(CN4CCCCC4)cc3OC)C2=O)ccc1OC] |
| CU0776 | 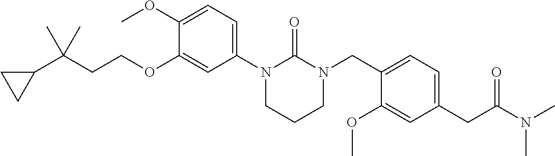 [SMILES: COc1cc(CC(=O)N(C)C)ccc1CN1CCCN(c2ccc(OC)c(OCCC(C)(C)C3CC3)c2)C1=O] |
| CU0777 | 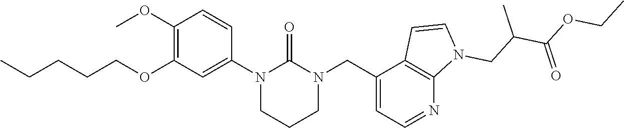 [SMILES: CCCCCOc1cc(N2CCCN(Cc3ccnc4c3ccn4CC(C)C(=O)OCC)C2=O)ccc1OC] |
| CU0778 | 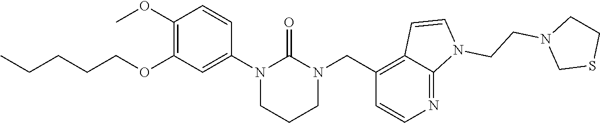 [SMILES: CCCCCOc1cc(N2CCCN(Cc3ccnc4c3ccn4CCN3CCSC3)C2=O)ccc1OC] |
| CU0779 | 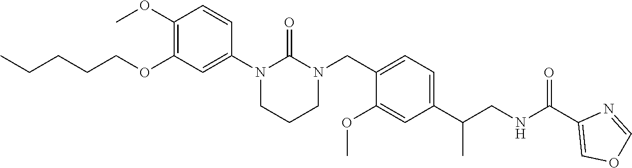 [SMILES: CCCCCOc1cc(N2CCCN(Cc3ccc(C(C)CNC(=O)c4cocn4)cc3OC)C2=O)ccc1OC] |
| CU0780 | 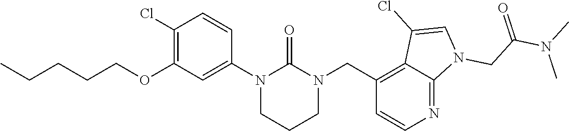 [SMILES: CCCCCOc1cc(N2CCCN(Cc3ccnc4c3c(Cl)cn4CC(=O)N(C)C)C2=O)ccc1Cl] |
| CU0781 | 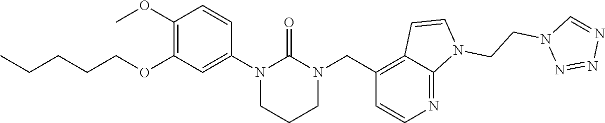 [SMILES: CCCCCOc1cc(N2CCCN(Cc3ccnc4c3ccn4CCn3cnnn3)C2=O)ccc1OC] |
| CU0782 | 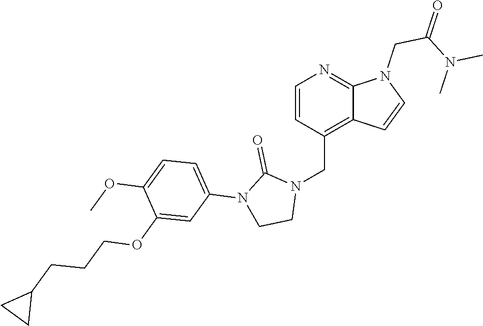 [SMILES: COc1ccc(N2CCN(Cc3ccnc4c3ccn4CC(=O)N(C)C)C2=O)cc1OCCCC1CC1] |
| CU0783 | 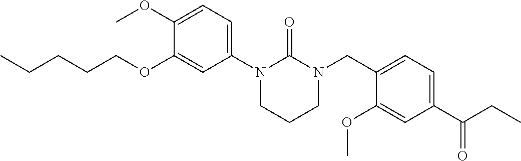 [SMILES: CCCCCOc1cc(N2CCCN(Cc3ccc(C(=O)CC)cc3OC)C2=O)ccc1OC] |
| CU0784 | 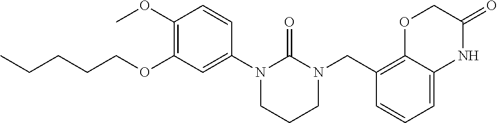 [SMILES: CCCCCOc1cc(N2CCCN(Cc3cccc4c3OCC(=O)N4)C2=O)ccc1OC] |
| CU0785 | 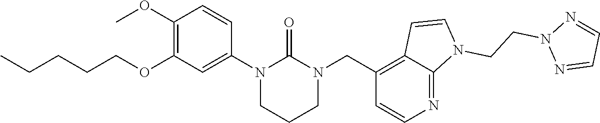 [SMILES: CCCCCOc1cc(N2CCCN(Cc3ccnc4c3ccn4CCn3nccn3)C2=O)ccc1OC] |
| CU0786 | 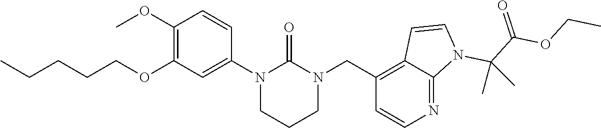 [SMILES: CCCCCOc1cc(N2CCCN(Cc3ccnc4c3ccn4C(C)(C)C(=O)OCC)C2=O)ccc1OC] |
| CU0787 | 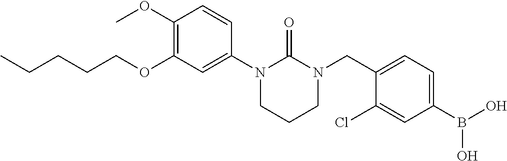 [SMILES: CCCCCOc1cc(N2CCCN(Cc3ccc(B(O)O)cc3Cl)C2=O)ccc1OC] |
| CU0788 | 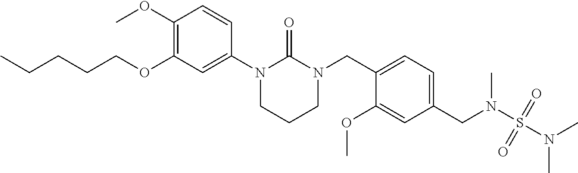 [SMILES: CCCCCOc1cc(N2CCCN(Cc3ccc(CN(C)S(=O)(=O)N(C)C)cc3OC)C2=O)ccc1OC] |
| CU0789 | 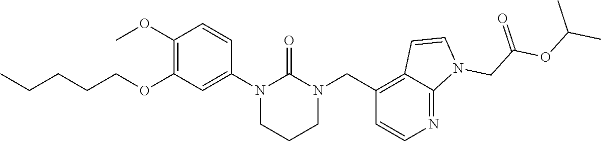 [SMILES: CCCCCOc1cc(N2CCCN(Cc3ccnc4c3ccn4CC(=O)OC(C)C)C2=O)ccc1OC] |
| CU0790 | 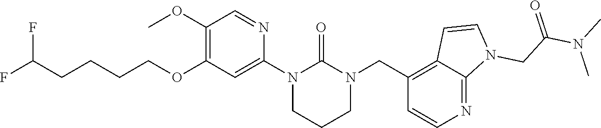 [SMILES: COc1cnc(N2CCCN(Cc3ccnc4c3ccn4CC(=O)N(C)C)C2=O)cc1OCCCCC(F)F] |
| CU0791 | 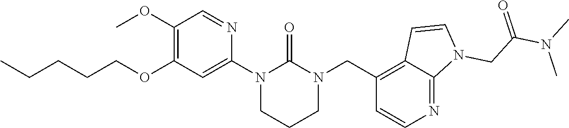 [SMILES: CCCCCOc1cc(N2CCCN(Cc3ccnc4c3ccn4CC(=O)N(C)C)C2=O)ncc1OC] |
| CU0792 | 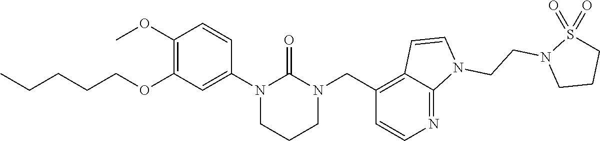 [SMILES: CCCCCOc1cc(N2CCCN(Cc3ccnc4c3ccn4CCN3CCCS3(=O)=O)C2=O)ccc1OC] |
| CU0793 | 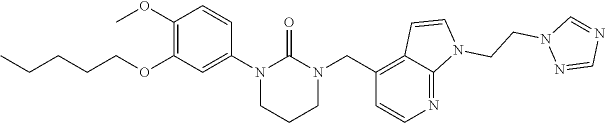 [SMILES: CCCCCOc1cc(N2CCCN(Cc3ccnc4c3ccn4CCn3cncn3)C2=O)ccc1OC] |
| CU0794 | 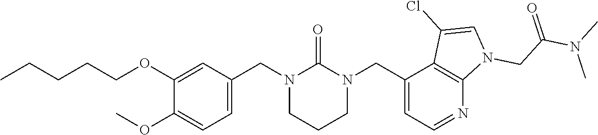 [SMILES: CCCCCOc1cc(CN2CCCN(Cc3ccnc4c3c(Cl)cn4CC(=O)N(C)C)C2=O)ccc1OC] |
| CU0795 | 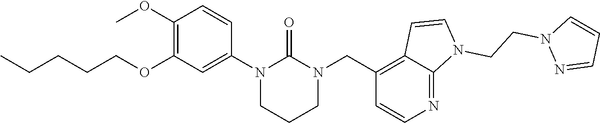 [SMILES: CCCCCOc1cc(N2CCCN(Cc3ccnc4c3ccn4CCn3cccn3)C2=O)ccc1OC] |
| CU0796 | 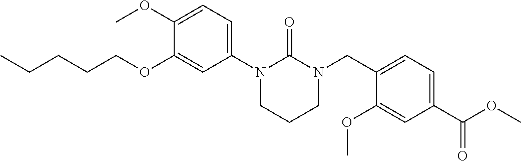 [SMILES: CCCCCOc1cc(N2CCCN(Cc3ccc(C(=O)OC)cc3OC)C2=O)ccc1OC] |
| CU0797 | 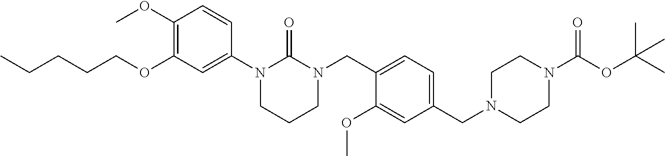 [SMILES: CCCCCOc1cc(N2CCCN(Cc3ccc(CN4CCN(C(=O)OC(C)(C)C)CC4)cc3OC)C2=O)ccc1OC] |
| CU0798 | 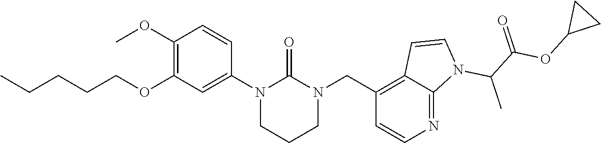 [SMILES: CCCCCOc1cc(N2CCCN(Cc3ccnc4c3ccn4C(C)C(=O)OC3CC3)C2=O)ccc1OC] |
| CU0799 | 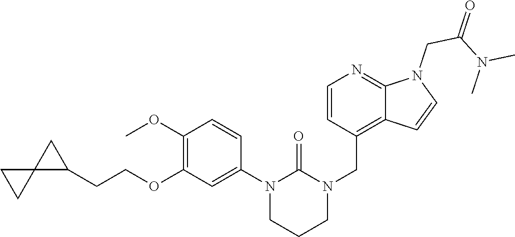 [SMILES: COc1ccc(N2CCCN(Cc3ccnc4c3ccn4CC(=O)N(C)C)C2=O)cc1OCCC1CC12CC2] |
| CU0800 | 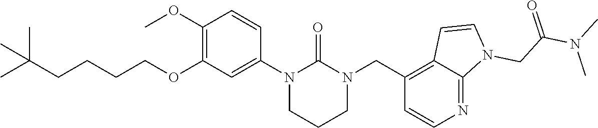 [SMILES: COc1ccc(N2CCCN(Cc3ccnc4c3ccn4CC(=O)N(C)C)C2=O)cc1OCCCCC(C)(C)C] |
| CU0801 | 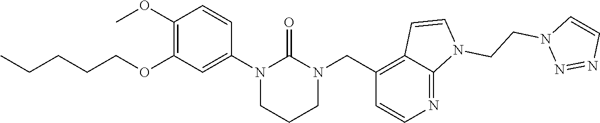 [SMILES: CCCCCOc1cc(N2CCCN(Cc3ccnc4c3ccn4CCn3ccnn3)C2=O)ccc1OC] |
| CU0802 | 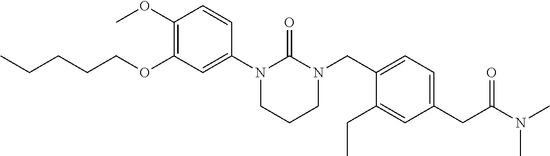 [SMILES: CCCCCOc1cc(N2CCCN(Cc3ccc(CC(=O)N(C)C)cc3CC)C2=O)ccc1OC] |
| CU0803 | 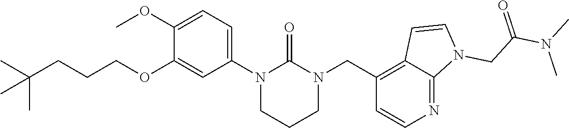 [SMILES: COc1ccc(N2CCCN(Cc3ccnc4c3ccn4CC(=O)N(C)C)C2=O)cc1OCCCC(C)(C)C] |
| CU0804 | 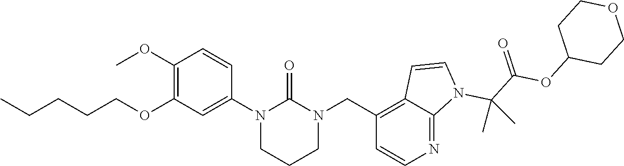 [SMILES: CCCCCOc1cc(N2CCCN(Cc3ccnc4c3ccn4C(C)(C)C(=O)OC3CCOCC3)C2=O)ccc1OC] |
| CU0805 | 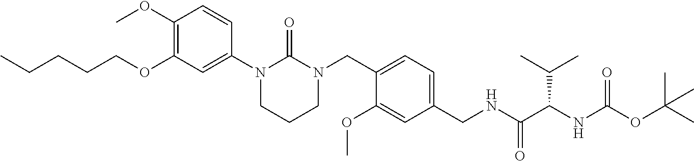 [SMILES: CCCCCOc1cc(N2CCCN(Cc3ccc(CNC(=O)[C@@H](NC(=O)OC(C)(C)C)C(C)C)cc3OC)C2=O)ccc1OC] |
| CU0806 | 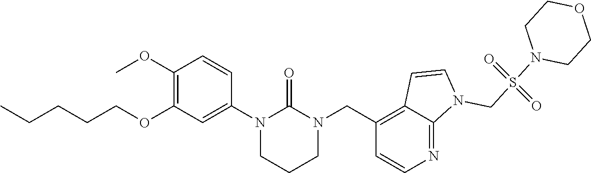 [SMILES: CCCCCOc1cc(N2CCCN(Cc3ccnc4c3ccn4CS(=O)(=O)N3CCOCC3)C2=O)ccc1OC] |
| CU0807 | 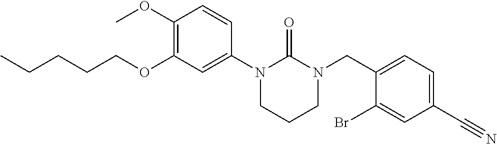 [SMILES: CCCCCOc1cc(N2CCCN(Cc3ccc(C#N)cc3Br)C2=O)ccc1OC] |
| CU0808 | 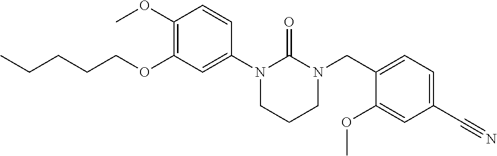 [SMILES: CCCCCOc1cc(N2CCCN(Cc3ccc(C#N)cc3OC)C2=O)ccc1OC] |
| CU0809 | 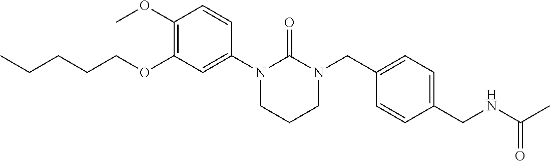 [SMILES: CCCCCOc1cc(N2CCCN(Cc3ccc(CNC(C)=O)cc3)C2=O)ccc1OC] |
| CU0810 | 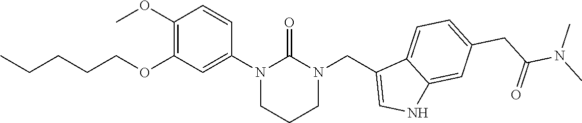 [SMILES: CCCCCOc1cc(N2CCCN(Cc3c[nH]c4cc(CC(=O)N(C)C)ccc34)C2=O)ccc1OC] |
| CU0811 | 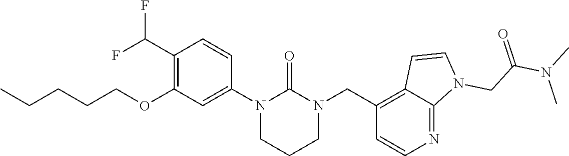 [SMILES: CCCCCOc1cc(N2CCCN(Cc3ccnc4c3ccn4CC(=O)N(C)C)C2=O)ccc1C(F)F] |
| CU0812 | 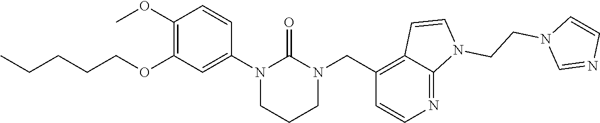 [SMILES: CCCCCOc1cc(N2CCCN(Cc3ccnc4c3ccn4CCn3ccnc3)C2=O)ccc1OC] |
| CU0813 | 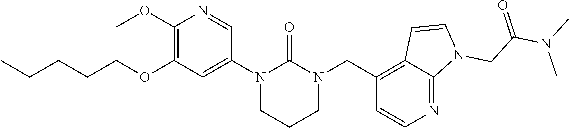 [SMILES: CCCCCOc1cc(N2CCCN(Cc3ccnc4c3ccn4CC(=O)N(C)C)C2=O)cnc1OC] |
| CU0814 | 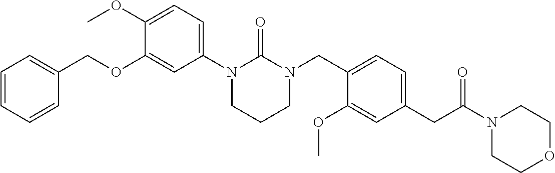 [SMILES: COc1cc(CC(=O)N2CCOCC2)ccc1CN1CCCN(c2ccc(OC)c(OCc3ccccc3)c2)C1=O] |
| CU0815 | 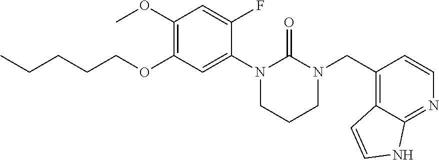 [SMILES: CCCCCOc1cc(N2CCCN(Cc3ccnc4[nH]ccc34)C2=O)c(F)cc1OC] |
| CU0816 | 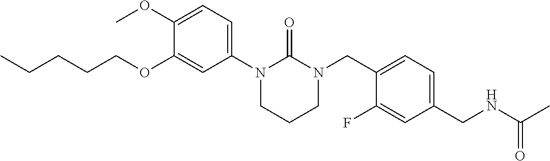 [SMILES: CCCCCOc1cc(N2CCCN(Cc3ccc(CNC(C)=O)cc3F)C2=O)ccc1OC] |
| CU0817 | 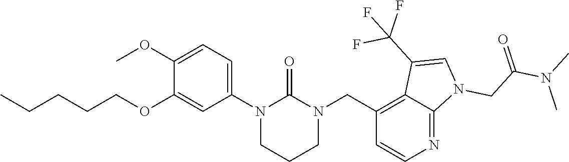 [SMILES: CCCCCOc1cc(N2CCCN(Cc3ccnc4c3c(C(F)(F)F)cn4CC(=O)N(C)C)C2=O)ccc1OC] |
| CU0818 | 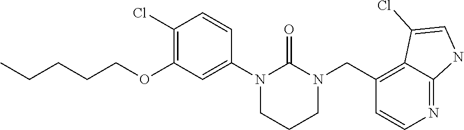 [SMILES: CCCCCOc1cc(N2CCCN(Cc3ccnc4[nH]cc(Cl)c34)C2=O)ccc1Cl] |
| CU0819 | 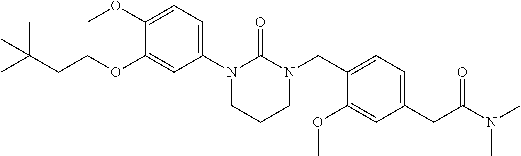 [SMILES: COc1cc(CC(=O)N(C)C)ccc1CN1CCCN(c2ccc(OC)c(OCCC(C)(C)C)c2)C1=O] |
| CU0820 | 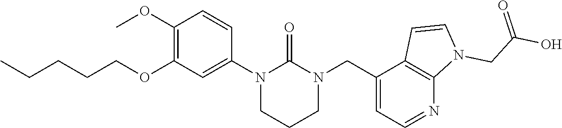 [SMILES: CCCCCOc1cc(N2CCCN(Cc3ccnc4c3ccn4CC(=O)O)C2=O)ccc1OC] |
| CU0821 | 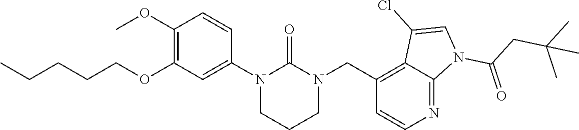 [SMILES: CCCCCOc1cc(N2CCCN(Cc3ccnc4c3c(Cl)cn4C(=O)CC(C)(C)C)C2=O)ccc1OC] |
| CU0822 | 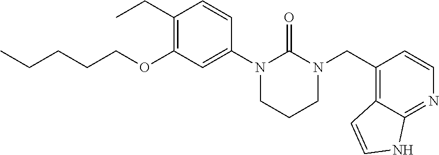 [SMILES: CCCCCOc1cc(N2CCCN(Cc3ccnc4[nH]ccc34)C2=O)ccc1CC] |
| CU0823 | 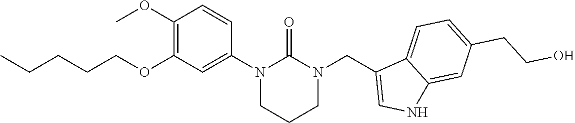 [SMILES: CCCCCOc1cc(N2CCCN(Cc3c[nH]c4cc(CCO)ccc34)C2=O)ccc1OC] |
| CU0824 | 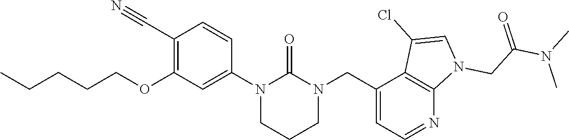 [SMILES: CCCCCOc1cc(N2CCCN(Cc3ccnc4c3c(Cl)cn4CC(=O)N(C)C)C2=O)ccc1C#N] |
| CU0825 | 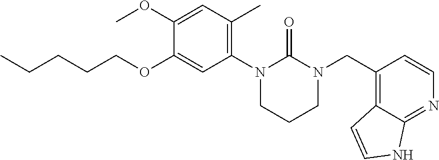 [SMILES: CCCCCOc1cc(N2CCCN(Cc3ccnc4[nH]ccc34)C2=O)c(C)cc1OC] |
| CU0826 | 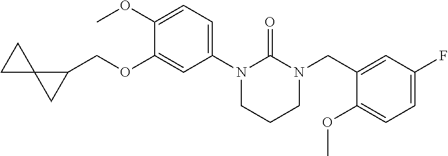 [SMILES: COc1ccc(F)cc1CN1CCCN(c2ccc(OC)c(OCC3CC34CC4)c2)C1=O] |
| CU0827 | 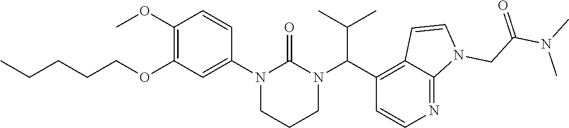 [SMILES: CCCCCOc1cc(N2CCCN(C(c3ccnc4c3ccn4CC(=O)N(C)C)C(C)C)C2=O)ccc1OC] |
| CU0828 | 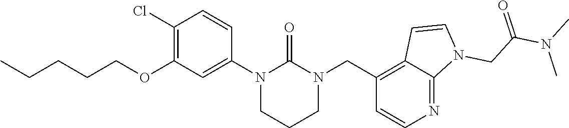 [SMILES: CCCCCOc1cc(N2CCCN(Cc3ccnc4c3ccn4CC(=O)N(C)C)C2=O)ccc1Cl] |
| CU0829 | 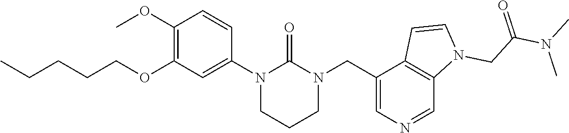 [SMILES: CCCCCOc1cc(N2CCCN(Cc3cncc4c3ccn4CC(=O)N(C)C)C2=O)ccc1OC] |
| CU0830 | 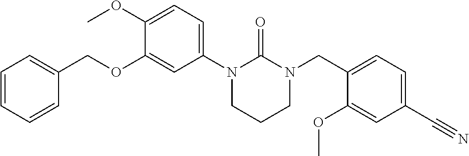 [SMILES: COc1cc(C#N)ccc1CN1CCCN(c2ccc(OC)c(OCc3ccccc3)c2)C1=O] |
| CU0831 | 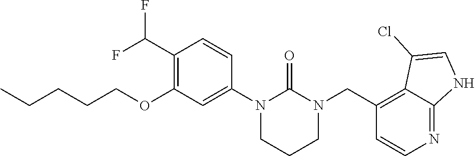 [SMILES: CCCCCOc1cc(N2CCCN(Cc3ccnc4[nH]cc(Cl)c34)C2=O)ccc1C(F)F] |
| CU0832 | 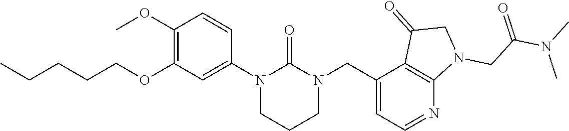 [SMILES: CCCCCOc1cc(N2CCCN(Cc3ccnc4c3C(=O)CN4CC(=O)N(C)C)C2=O)ccc1OC] |
| CU0833 | 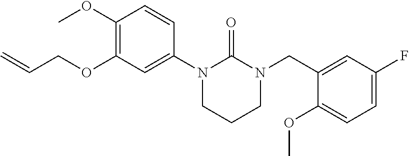 [SMILES: C=CCOc1cc(N2CCCN(Cc3cc(F)ccc3OC)C2=O)ccc1OC] |
| CU0834 | 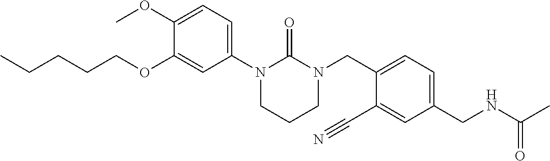 [SMILES: CCCCCOc1cc(N2CCCN(Cc3ccc(CNC(C)=O)cc3C#N)C2=O)ccc1OC] |
| CU0835 | 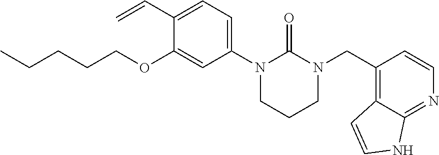 [SMILES: C=Cc1ccc(N2CCCN(Cc3ccnc4[nH]ccc34)C2=O)cc1OCCCCC] |
| CU0836 | 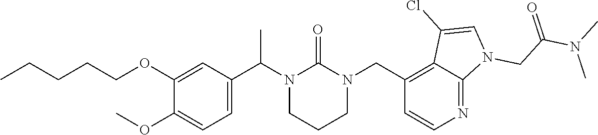 [SMILES: CCCCCOc1cc(C(C)N2CCCN(Cc3ccnc4c3c(Cl)cn4CC(=O)N(C)C)C2=O)ccc1OC] |
| CU0837 | 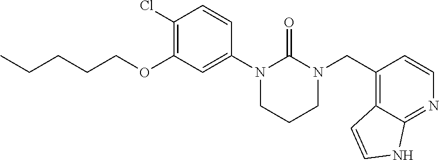 [SMILES: CCCCCOc1cc(N2CCCN(Cc3ccnc4[nH]ccc34)C2=O)ccc1Cl] |
| CU0838 | 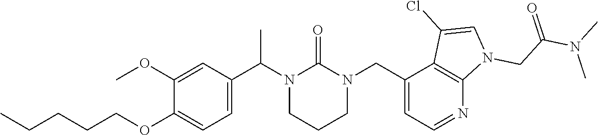 [SMILES: CCCCCOc1ccc(C(C)N2CCCN(Cc3ccnc4c3c(Cl)cn4CC(=O)N(C)C)C2=O)cc1OC] |
| CU0839 | 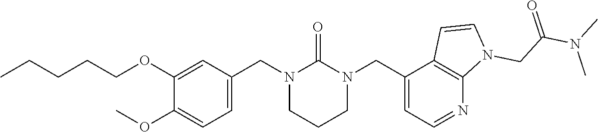 [SMILES: CCCCCOc1cc(CN2CCCN(Cc3ccnc4c3ccn4CC(=O)N(C)C)C2=O)ccc1OC] |
| CU0840 | 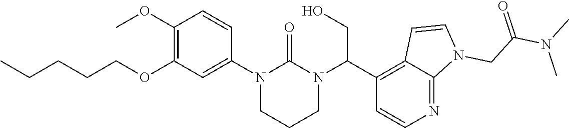 [SMILES: CCCCCOc1cc(N2CCCN(C(CO)c3ccnc4c3ccn4CC(=O)N(C)C)C2=O)ccc1OC] |
| CU0841 | 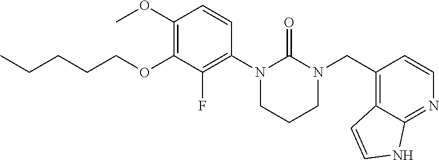 [SMILES: CCCCCOc1c(OC)ccc(N2CCCN(Cc3ccnc4[nH]ccc34)C2=O)c1F] |
| CU0842 | 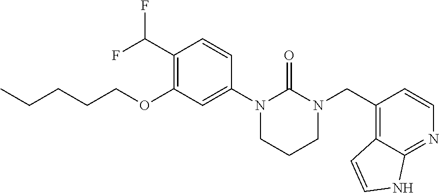 [SMILES: CCCCCOc1cc(N2CCCN(Cc3ccnc4[nH]ccc34)C2=O)ccc1C(F)F] |
| CU0843 | 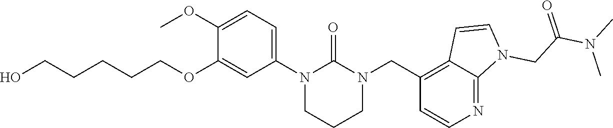 [SMILES: COc1ccc(N2CCCN(Cc3ccnc4c3ccn4CC(=O)N(C)C)C2=O)cc1OCCCCCO] |
| CU0844 | 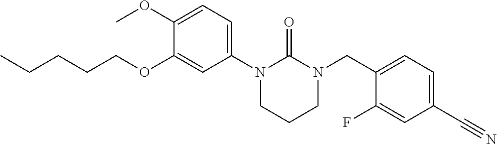 [SMILES: CCCCCOc1cc(N2CCCN(Cc3ccc(C#N)cc3F)C2=O)ccc1OC] |
| CU0845 | 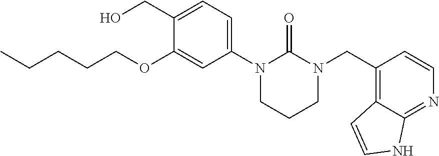 [SMILES: CCCCCOc1cc(N2CCCN(Cc3ccnc4[nH]ccc34)C2=O)ccc1CO] |
| CU0846 | 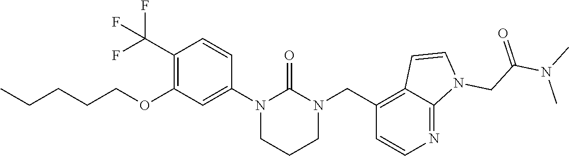 [SMILES: CCCCCOc1cc(N2CCCN(Cc3ccnc4c3ccn4CC(=O)N(C)C)C2=O)ccc1C(F)(F)F] |
| CU0847 | 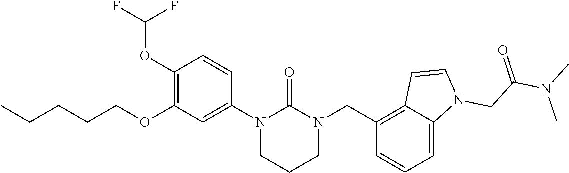 [SMILES: CCCCCOc1cc(N2CCCN(Cc3cccc4c3ccn4CC(=O)N(C)C)C2=O)ccc1OC(F)F] |

### Generic Structure—Formula (IIa)

In some embodiments, a C5 inhibitor compound of the present disclosure has a structure according to Formula (Ha):

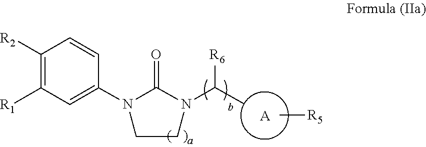

or a pharmaceutically acceptable salt thereof;
wherein a is 1, 2 or 3;
wherein b is 1 or 2;
wherein R1 is a C1-C7 alkyl group, C1-C7 alkoxy group, wherein R1 is optionally substituted with one or more substituents, such as alkyl, alkoxyl, halogen, a phenyl group, a cyclic group, a bicyclic group, an alkenyl group, or an alkynyl group, wherein each of these groups is optionally further substituted, such as with at least one halogen, an alkyl group, or an alkoxyl group;
wherein R₂ is a C₁-C₄ alkyl group, a C₁-C₄ alkoxy group, or a C₃-C₅ cycloalkyl group; wherein R₆ is independently hydrogen, OH, a C₁-C₃ alkyl group, or a C₁-C₃ alkoxyl group; wherein

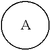

comprises at least one aryl ring or a heteroaryl ring, optionally substituted with one or more R₅ groups;
wherein each R₅ is independently a suitable functional group, such as hydrogen, a halogen, a C₁-C₄ alkyl group, a C₁-C₄ alkoxy group, a C₃-C₆ cycloalkyl group, a C₃-C₆ heterocycle group, a pyridine or alkylpyridine optionally substituted with one or more C₁-C₄ alkyl groups, a pyrrolidinone or alkylpyrrolidinone optionally substituted with one or more C₁-C₄ alkyl groups, a triazole or alkyltriazole optionally substituted with a C₁-C₄ alkyl group, —(C₁-C₃ alkyl)-CO—N(R₁₅)₂, —(C₁-C₃ alkyl)-CO—R₁₆, or

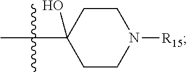

wherein each R₁₅ is independently selected from hydrogen or a C1-C4 alkyl group; and wherein R₁₆ is selected from a group consisting of a pyrrolidine, a morpholine, a piperazine, and an oxazepane; and wherein each R₁₆ is optionally substituted with one or more substituents selected from the group consisting of: C₁-C₄ alkyl group, a C₃-C₅ cycloalkyl group, C₁-C₃ hydroxyalkyl group, C₁-C₄ alkoxy group, C₁-C₄ alkylmethoxy group, C₁-C₄ alkylethoxy group, —(C1-C3 alkyl)-N(R₁₅)₂, an C₁-C₃ alkylpyrrolidine group, an acetyl group, and an oxo group.

In some embodiments, R₁ is

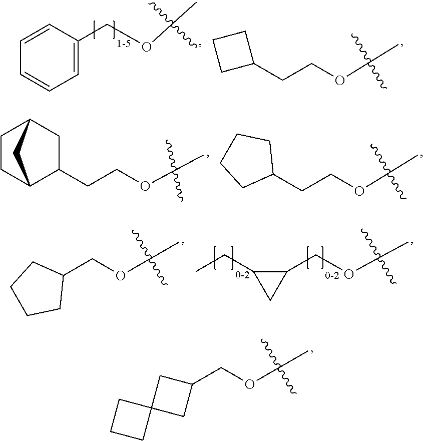

or their substituted derivatives.

In some embodiments,

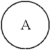

is a bicyclic group, optionally comprises 1 to 3 heteroatoms, such as nitrogen and/or oxygen.

Non-limiting examples of C5 inhibitor compounds having a structure according to Formula (IIa) include CU0001, CU0002, CU0003, CU0004, CU0005, CU0006, CU0007, CU0008, CU0009, CU0010, CU0011, CU0012, CU0013, CU0014, CU0015, CU0016, CU0017, CU0018, CU0019, CU0020, CU0021, CU0022, CU0023, CU0024, CU0025, CU0026, CU0027, CU0028, CU0029, CU0030, CU0031, CU0032, CU0033, CU0034, CU0035, CU0036, CU0037, CU0038, CU0039, CU0040, CU0041, CU0042, CU0043, CU0044, CU0045, CU0046, CU0047, CU0048, CU0049, CU0050, CU0051, CU0052, CU0053, CU0054, CU0055, CU0056, CU0057, CU0058, CU0059, CU0060, CU0061, CU0062, CU0063, CU0064, CU0065, CU0066, and CU0067.

### Generic Structure—Formula (IIb)

In some embodiments, a C5 inhibitor compound of the present disclosure has a structure according to Formula (IIb):

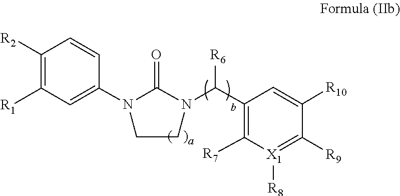

or a pharmaceutically acceptable salt thereof;
wherein a is 1, 2 or 3;
wherein b is 1 or 2;
wherein X1 is carbon or nitrogen;
wherein R₁ is a C₁-C₇ alkyl group, C₁-C₇ alkoxy group, wherein R₁ is optionally substituted with one or more substituents, such as alkyl, alkoxyl, halogen, a phenyl group, a cyclic group, a bicyclic group, an alkenyl group, or an alkynyl group, wherein each of these groups is optionally further substituted, such as with at least one halogen, an alkyl group, or an alkoxyl group;
wherein R₂ is a C₁-C₄ alkyl group, a C₁-C₄ alkoxy group, or a C₃-C₅ cycloalkyl group;
wherein R₆ is independently hydrogen, OH, a C₁-C₃ alkyl group, or a C₁-C₃ alkoxyl group;
wherein R₇ is any suitable functional group, such as hydrogen, a C₁-C₃ alkyl group, C₁-C₃ alkoxy, a C₃-C₅ cycloalkyl group, or a halogen;
wherein R₈ is any suitable functional group, such as hydrogen, a halogen or a C₁-C₃ alkyl group;
wherein R₁₀ is any suitable functional group, such as hydrogen, a halogen, a C₁-C₃ alkyl group, or a cyclic group;
wherein R₉ is any suitable functional group, such as hydrogen, a halogen, a C₁-C₆ alkyl group, an alkoxy group, an aryl group, a heteroaryl group, —(C₁-C₂ alkyl)-CO—NR₁₁R₁₂, —(C₁-C₂ alkyl)-O—NR₁₁R₁₂, an amine group optionally substituted with one or two alkyl groups, a cyclic or heterocyclic group optionally substituted with one or more alkyl groups, any group that comprises a carbamate group (—NH—COO—), any group that comprises carboxyl group (—COO—), any group that comprises a carbonyl group (—CO—), any group that comprises amide group (—CO—NH—) optionally substituted with one alkyl group, any group that comprises —CH═N— optionally substituted with an alkyl group or an amine group, or any group that comprises —C≡N;
wherein each R9 group is optionally further substituted with one or more halogens, —OH, alkyl, or alkoxyl groups; and
wherein R₁₁ and R₁₂ are independently any suitable functional group, such as hydrogen, a C₁-C₆ alkyl, C₁-C₃ alkoxy, a cyclic or heterocyclic group, or wherein Ru and Ru combine to form a 5-7 member ring and one or more carbons in the ring can be replaced with N or O, wherein the ring may comprise a carbonyl group (C═O).

In some embodiments, R₁ is

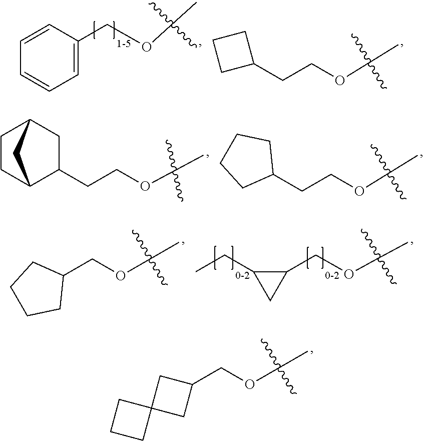

or their substituted derivatives.

Non-limiting examples of C5 inhibitor compounds having a structure according to Formula (IIb) include CU0001, CU0002, CU0003, CU0004, CU0005, CU0006, CU0007, CU0008, CU0009, CU0010, CU0011, CU0012, CU0013, CU0014, CU0015, CU0016, CU0017, CU0018, CU0100, CU0101, CU0102, CU0103, CU0104, CU0105, CU0106, CU0107, CU0108, CU0109, CU0110, CU0111, CU0112, CU0113, CU0114, CU0115, CU0116, CU0117, CU0118, CU0119, CU0120, CU0121, CU0122, CU0123, CU0124, CU0125, CU0126, CU0127, CU0128, CU0129, CU0130, CU0131, CU0132, CU0133, CU0134, CU0135, CU0136, CU0137, CU0138, CU0139, CU0140, CU0141, CU0142, CU0143, CU0144, CU0145, CU0146, CU0147, CU0148, CU0149, CU0150, CU0151, CU0152, CU0153, CU0154, CU0155, CU0156, CU0157, CU0158, CU0159, CU0160, CU0161, CU0162, CU0163, CU0164, CU0165, CU0166, CU0167, CU0168, CU0169, CU0170, CU0171, CU0172, CU0173, CU0174, CU0175, CU0176, CU0177, CU0178, CU0179, CU0180, CU0181, CU0182, CU0183, CU0184, CU0185, CU0186, CU0187, CU0188, CU0189, CU0190, CU0191, CU0192, CU0193, CU0194, CU0195, CU0196, CU0197, CU0198, CU0199, CU0200, CU0201, CU0202, CU0203, CU0204, CU0205, CU0206, CU0207, CU0208, CU0209, CU0210, CU0211, CU0212, CU0213, CU0214, CU0215, CU0216, CU0217, CU0218, CU0219, CU0220, CU0221, CU0222, CU0223, CU0224, CU0225, CU0226, and CU0227.

### Generic Structure—Formula (IIc)

In some embodiments, a C5 inhibitor compound of the present disclosure has a structure according to Formula (IIc):

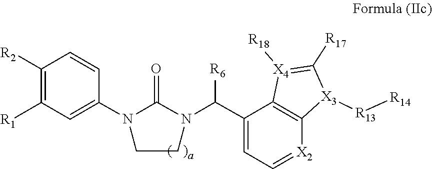

or a pharmaceutically acceptable salt thereof;
wherein a is 1, 2 or 3;
wherein X₂ is carbon or nitrogen;
wherein X₃ is nitrogen or oxygen, and R₁₃ and R₁₄ are not present when X₃ is oxygen;
wherein X₄ is carbon, nitrogen, or oxygen, and R₁₈ is not present when X₄ is oxygen;
wherein R₁ is a C₁-C₇ alkyl group or C₁-C₇ alkoxy group, wherein R₁ is optionally substituted with one or more substituents selected from the group consisting of an alkyl, an alkoxyl, a halogen, a phenyl group, a cyclic group, a bicyclic group, an alkenyl group, and, an alkynyl group, wherein each of the one or more substituents is optionally further substituted with at least one halogen, an alkyl group, or an alkoxyl group;
wherein R₂ is a branched or linear C₁-C₄ alkoxy group, or a C₃-C₅ cycloalkyl group;
wherein R₆ is hydrogen, OH, a C₁-C₃ alkyl group, or a C₁-C₃ alkoxyl group; wherein R₁₃ is a bond, a C₁-C₃ alkyl group a carbonyl group (—CO—), alkenyl group (—CH═CH—) optionally substituted with one or two alkyl groups, or amide group (—CO—NH—) optionally substituted with one alkyl group;
wherein R₁₄ is any suitable functional group, such as hydrogen, a pyridine optionally substituted with one or more C₁-C₄ alkyl groups, an amine group optionally substituted with one or two alkyl groups, a cyclic or heterocyclic group optionally substituted with one or more alkyl groups, any group that comprises a carbonyl group (—CO—), amide group (—CO—NH—) optionally substituted with one alkyl group, —CH═N— optionally substituted with an alkyl group or an amine group, a pyrrolidinone optionally substituted with one or more C₁-C₄ alkyl groups, a triazole optionally substituted with a C₁-C₄ alkyl group, —CO—N(R₁₅)₂, —CO—R₁₆

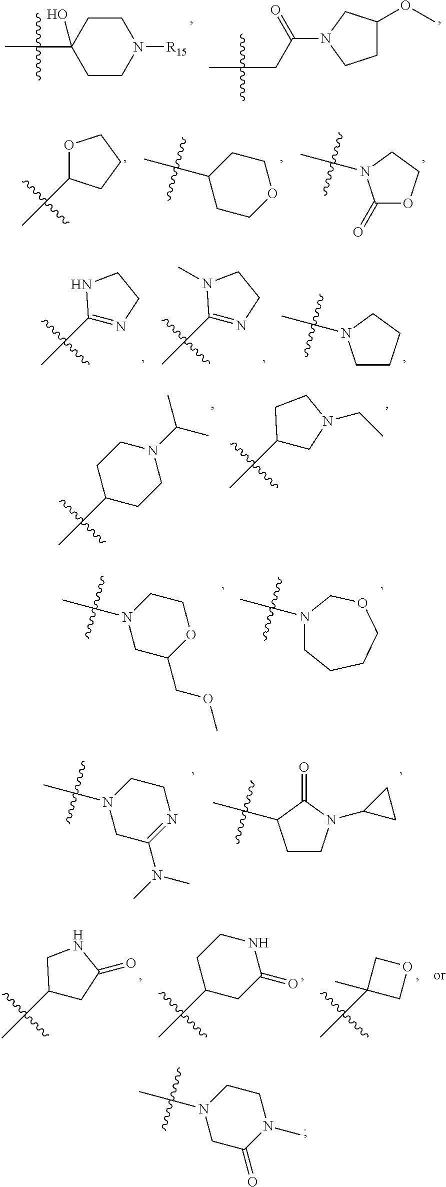

wherein each R₁₅ is independently any suitable functional group, such as hydrogen or a C₁-C₄ alkyl group; and
wherein R₁₆ is any suitable functional group, such as a morpholine, a piperazine, and an oxazepane; wherein each R₁₆ is optionally substituted with one or more substituents selected from the group consisting of: C₁-C₄ alkyl group, a C₃-C₅ cycloalkyl group, C₁-C₃ hydroxyalkyl group, C₁-C₄ alkoxy group, C₁-C₄ alkylmethoxy group, C₁-C₄ alkylethoxy group, —(C₁-C₃ alkyl)-N(R₁₅)₂, an C₁-C₃ alkylpyrrolidine group, an acetyl group, and an oxo group;
wherein R17 is any suitable functional group, such as hydrogen or a C1-C4 alkyl group, and
wherein R₁₅ is any suitable functional group, such as hydrogen, a halogen, or an alkyl group.

In some embodiments, when X₂ is nitrogen, X₃ is nitrogen and X₄ is carbon.

In some embodiments, R₁ is

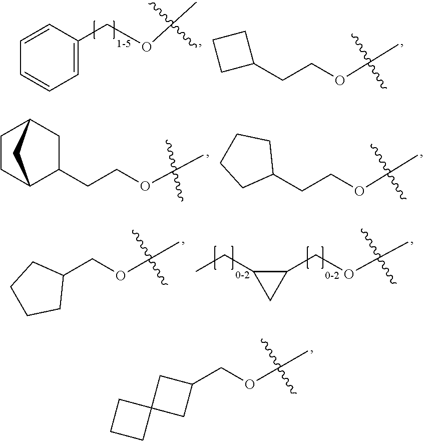

or their substituted derivatives.

Non-limiting examples of C5 inhibitor compounds having a structure according to Formula (IIc) include CU0019, CU0020, CU0021, CU0022, CU0023, CU0024, CU0025, CU0026, CU0027, CU0028, CU0029, CU0030, CU0031, CU0032, CU0033, CU0034, CU0035, CU0036, CU0037, CU0038, CU0039, CU0040, CU0041, CU0042, CU0043, CU0044, CU0045, CU0046, CU0047, CU0048, CU0049, CU0050, CU0051, CU0052, CU0053, CU0054, CU0055, CU0056, CU0057, CU0058, CU0059, CU0060, CU0061, CU0062, CU0063, CU0064, CU0065, CU0066, and CU0067.

### Generic Structure—Formula (IId)

In some embodiments, a C5 inhibitor compound of the present disclosure has a structure according to Formula (IId):

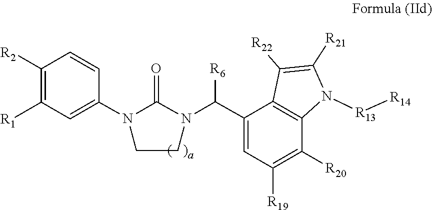

or a pharmaceutically acceptable salt thereof;
wherein a is 1, 2 or 3;
wherein R₁ is a C₁-C₇ alkyl group, C₁-C₇ alkoxy group, wherein R₁ is optionally substituted with one or more substituents, such as alkyl, alkoxyl, halogen, a phenyl group, a cyclic group, a bicyclic group, an alkenyl group, or an alkynyl group, wherein each of these groups is optionally further substituted, such as with at least one halogen, an alkyl group, or an alkoxyl group;
wherein R₂ is a branched or linear C₁-C₄ alkoxy group, or a C₃-C₅ cycloalkyl group;
wherein R₆ is hydrogen, OH, a C₁-C₃ alkyl group, or a C₁-C₃ alkoxyl group,
wherein R₁₃ is a bond or a C₁-C₃ alkyl group;
wherein R₁₄ is any suitable functional group, such as hydrogen, a pyridine optionally substituted with one or more C₁-C₄ alkyl groups, an amine group optionally substituted with one or two alkyl groups, a cyclic or heterocyclic group optionally substituted with one or more alkyl groups, any group that comprises a carbonyl group (—CO—), amide group (—CO—NH—) optionally substituted with one alkyl group, —CH═N— optionally substituted with an alkyl group or an amine group, a pyrrolidinone optionally substituted with one or more C₁-C₄ alkyl groups, a triazole optionally substituted with a C₁-C₄ alkyl group, —CO—N(R₁₅)₂, —CO—R₁₆,

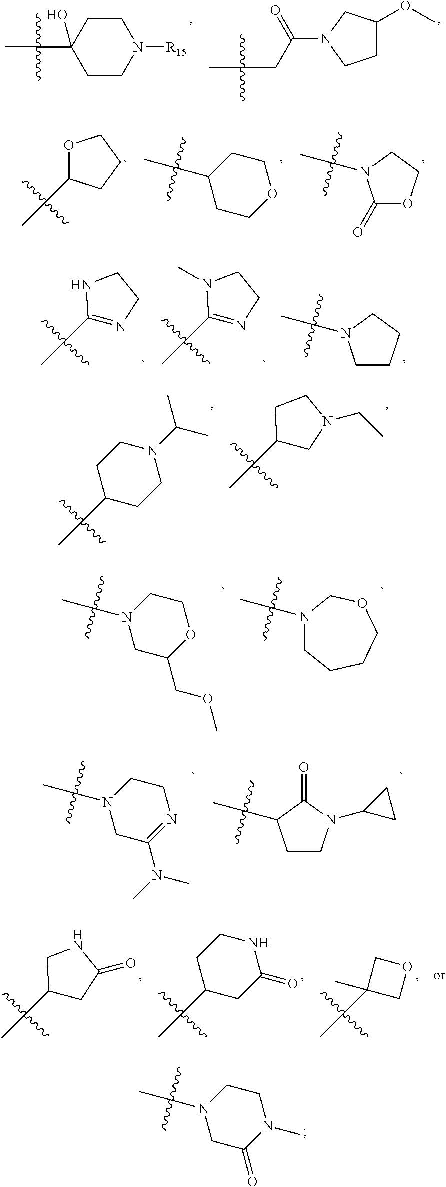

wherein each R₁₅ is independently any suitable functional group such as hydrogen or a C₁-C₄ alkyl group;
wherein R₁₆ is any suitable functional group such as a morpholine, a piperazine, and an oxazepane; wherein each R₁₆ is optionally substituted with one or more substituents selected from the group consisting of: C₁-C₄ alkyl group, a C₃-C₅ cycloalkyl group, C₁-C₃ hydroxyalkyl group, C₁-C₄ alkoxy group, C₁-C₄ alkylmethoxy group, C₁-C₄ alkylethoxy group, —(C₁-C₃ alkyl)-N(R₁₅)₂, an C₁-C₃ alkylpyrrolidine group, an acetyl group, and an oxo group;
wherein R₁₇ is any suitable functional group such as hydrogen or a C₁-C₄ alkyl group;
wherein R₁₉ is any suitable functional group such as hydrogen, an alkyl, or a halogen;
wherein R₂₀ is any suitable functional group hydrogen, an alkyl, or a halogen;
wherein R₂₁ is any suitable functional group hydrogen, an alkyl, or a halogen; and
wherein R₂₂ is any suitable functional group hydrogen, an alkyl, or a halogen.

In some embodiments, R₁ is

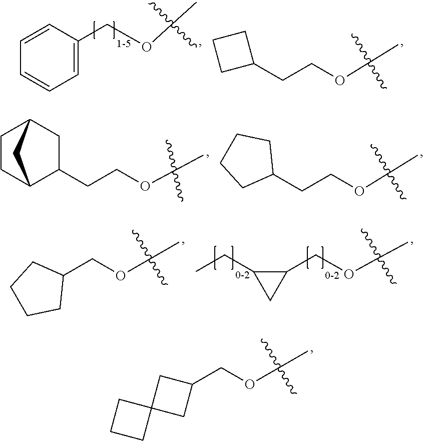

or their substituted derivatives.

Non-limiting examples of C5 inhibitor compounds having a structure according to Formula (IV) include CU0019, CU0020, CU0021, CU0022, CU0023, CU0024, CU0025, CU0026, CU0027, CU0028, CU0029, CU0030, CU0031, CU0228, CU0229, CU0230, CU0231, CU0232, CU0233, CU0234, CU0235, CU0236, CU0237, CU0238, CU0239, CU0240, CU0241, CU0242, CU0243, CU0244, CU0245, CU0246, and CU0247.

### Generic Structure—Formula (IId1)

In some embodiments, a C5 inhibitor compound of the present disclosure has a structure according to Formula (IId1):

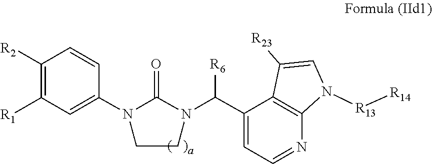

or a pharmaceutically acceptable salt thereof;
wherein a is 1, 2 or 3;
wherein R₁ is a C₁-C₇ alkyl group, C₁-C₇ alkoxy group, wherein R₁ is optionally substituted with one or more substituents, such as alkyl, alkoxyl, halogen, a phenyl group, a cyclic group, a bicyclic group, an alkenyl group, or an alkynyl group, wherein each of these groups is optionally further substituted, such as with at least one halogen, an alkyl group, or an alkoxyl group;
wherein R₂ is a branched or linear C₁-C₄ alkoxy group, or a C₃-C₅ cycloalkyl group;
wherein R₆ is hydrogen, OH, a C₁-C₃ alkyl group, or a C₁-C₃ alkoxyl group;
wherein R₁₃ is a bond, a C₁-C₃ alkyl group, a group that comprises a carbonyl group, cyclic group, or heterocyclic group;
wherein R₁₄ is any suitable functional group, such as hydrogen, a pyridine optionally substituted with one or more C₁-C₄ alkyl groups, an amine group optionally substituted with one or two alkyl groups, a cyclic or heterocyclic group optionally substituted with one or more alkyl groups, any group that comprises a carbonyl group (—CO—), amide group (—CO—NH—) optionally substituted with one alkyl group, —CH═N— optionally substituted with an alkyl group or an amine group, a pyrrolidinone optionally substituted with one or more C₁-C₄ alkyl groups, a triazole optionally substituted with a C₁-C₄ alkyl group, —CO—N(R₅)₂, —CO—R₁₆,

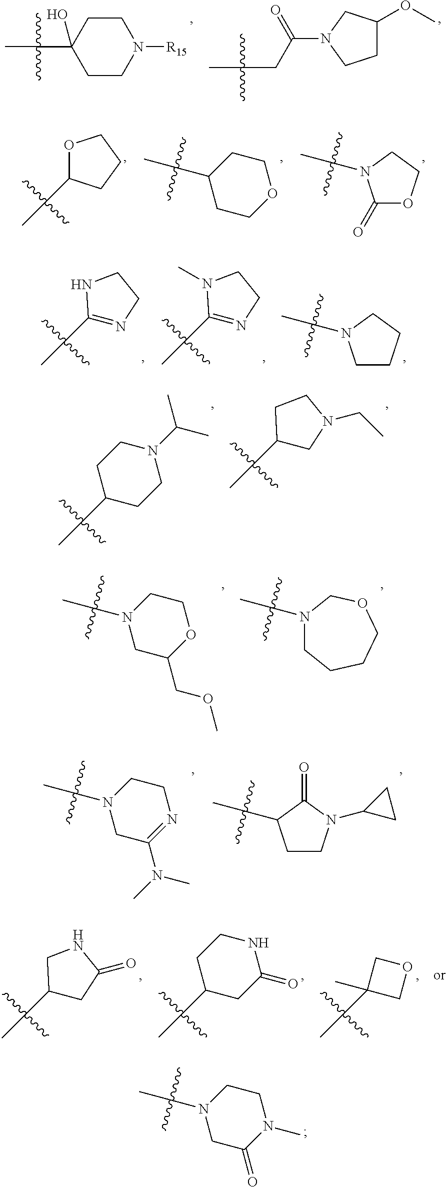

wherein each R₁₅ is independently any suitable functional group such as hydrogen or a C₁-C₄ alkyl group; and wherein R₁₆ is selected from a group consisting of a morpholine, a piperazine, and an oxazepane; wherein each R₁₆ is optionally substituted with one or more substituents selected from the group consisting of: C₁-C₄ alkyl group, a C₃-C₅ cycloalkyl group, C₁-C₃ hydroxyalkyl group, C₁-C₄ alkoxy group, C₁-C₄ alkylmethoxy group, C₁-C₄ alkylethoxy group, —(C₁-C₃ alkyl)-N(R₁₅)₂, an C₁-C₃ alkylpyrrolidine group, an acetyl group, and an oxo group;
wherein R₁₇ is any suitable functional group such as hydrogen or a C₁-C₄ alkyl group; and
wherein R₂₃ is any suitable functional group such as hydrogen, an alkyl, or a halogen.

In some embodiments, R₁ is

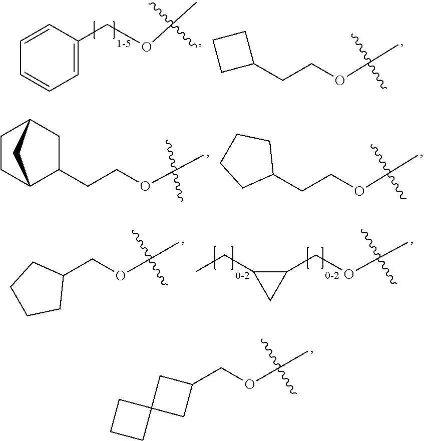

or their substituted derivatives.

Non-limiting examples of C5 inhibitor compounds having a structure according to Formula (IId1) include CU0032, CU0033, CU0034, CU0035, CU0036, CU0037, CU0038, CU0039, CU0040, CU0041, CU0042, CU0043, CU0044, CU0045, CU0046, CU0047, CU0048, CU0049, CU0050, CU0051, CU0052, CU0053, CU0054, CU0055, CU0056, CU0057, CU0058, CU0059, CU0060, CU0061, CU0062, CU0248, CU0249, CU0250, CU0251, CU0252, CU0253, CU0254, CU0255, CU0256, CU0257, CU0258, CU0259, CU0260, CU0261, and CU0262.

### Generic Structure—Formula (IIe)

In some embodiments, a C5 inhibitor compound of the present disclosure has a structure according to Formula (IIe):

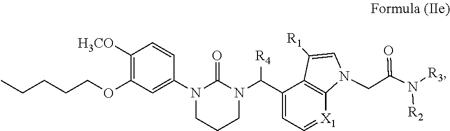

or a pharmaceutically acceptable salt thereof;
wherein X1 is CH or N;
wherein R1 is H, halogen (such as Cl, F, Br or I), —CN, —CF3, or a C1-C3 alkyl group;
wherein R2 or R3, independently, is H, alkyl, aryl, heteroaryl, cyclic alkyl, heterocyclic alkyl, a multicyclic alkyl group, or a hetero multicyclic alkyl group, wherein the alkyl, aryl, heteroaryl group, cyclic alkyl, heterocyclic alkyl, multicyclic alkyl, or hetero multicyclic alkyl group is optional substituted; optionally, R2 and R3, together with the nitrogen they are attached, form a 3 to 8 membered heterocyclic group, wherein the heterocyclic group may be optionally substituted; and wherein R4 is H or a C1-C3 alkyl group.

In some embodiments, R2 and R3 are both C1-C3 alkyl groups.

In some embodiments, R4 is H.

Non-limiting examples of C5 inhibitor compounds having a structure according to Formula (IIe) include CU0025, CU0028, CU0029, CU0030, CU0031, CU0035, CU0043, CU0046, CU0048, CU0049, CU0050, CU0051, CU0053, CU0056, CU0057, CU0060, CU0062, CU0231, CU0232, CU0235, CU0239, CU0243, CU0244, CU0245, CU0246, CU0247, CU0255, CU0257, CU0258, CU0260, CU0261, CU0504, CU0506, CU0508, CU0509, CU0510, CU0518, CU0519, CU0521, CU0526, CU0528, CU0529, CU0533, CU0534, CU0535, CU0538, CU0539, CU0540, CU0541, CU0543, CU0549, CU0553, CU0560, CU0561, CU0567, CU0602, CU0603, CU0747, and CU0817.

### Generic Structure—Formula (IIf)

In some embodiments, a C5 inhibitor compound of the present disclosure has a structure according to Formula (IIf):

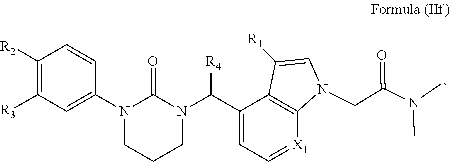

or a pharmaceutically acceptable salt thereof;
wherein X1 is CH or N;
wherein R1 is H, halogen (such as Cl, F, Br or I), —CN, —CF3, or a C1-C3 alkyl group;
wherein R2 or R3, independently, is alkyl, cyclic alkyl, alkenyl, alkynyl, halogen, hydroxyl, alkoxyl, ether, CN, amine, amide, aryl, or heteroaryl, wherein the alkyl, cyclic alkyl, alkenyl, alkynyl, alkoxyl, ether, amine, aryl, or heteroaryl group is optionally substituted; and wherein R4 is H or a C1-C3 alkyl group.

In some embodiments, R2 and R3 are both alkoxyl groups.

In some embodiments, R2 is —OCH3.

In some embodiments, R4 is H.

Non-limiting examples of C5 inhibitor compounds having a structure according to Formula (IIf) include CU0025, CU0026, CU0027, CU0035, CU0036, CU0231, CU0232, CU0252, CU0253, CU0256, CU0258, CU0259, CU0261, CU0262, CU0508, CU0515, CU0516, CU0532, CU0535, CU0543, CU0582, CU0591, CU0595, CU0602, CU0606, CU0610, CU0625, CU0681, CU0707, CU0737, CU0747, CU0752, CU0761, CU0764, CU0765, CU0767, CU0780, CU0790, CU0799, CU0800, CU0803, CU0811, CU0828, CU0843, CU0846, and CU0847.

3). Substituted Cyclic-urea C5-interacting Compounds

In some embodiments, C5-interacting compounds of the present disclosure may include any of the compounds listed in Table 3, including SC0001-SC0072 and SC0100-SC0232.

TABLE 3
Substituted Cyclic-urea C5-interacting compounds

| Compound | |
|---|---|
| Number | Structure |
| SC0001 | 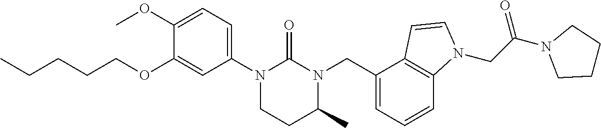 [SMILES: CCCCCOc1cc(N2CC[C@H](C)N(Cc3cccc4c3ccn4CC(=O)N3CCCC3)C2=O)ccc1OC] |
| SC0002 | 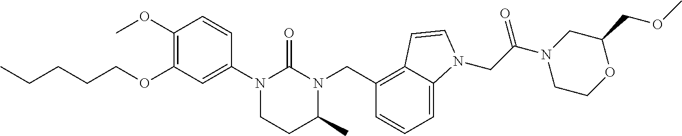 [SMILES: CCCCCOc1cc(N2CC[C@H](C)N(Cc3cccc4c3ccn4CC(=O)N3CCO[C@H](COC)C3)C2=O)ccc1OC] |
| SC0003 | 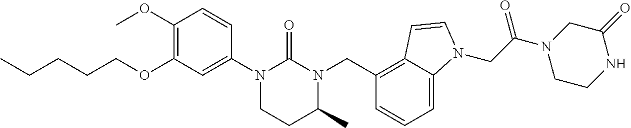 [SMILES: CCCCCOc1cc(N2CC[C@H](C)N(Cc3cccc4c3ccn4CC(=O)N3CCNC(=O)C3)C2=O)ccc1OC] |
| SC0004 | 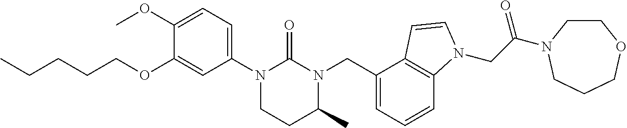 [SMILES: CCCCCOc1cc(N2CC[C@H](C)N(Cc3cccc4c3ccn4CC(=O)N3CCCOCC3)C2=O)ccc1OC] |
| SC0005 | 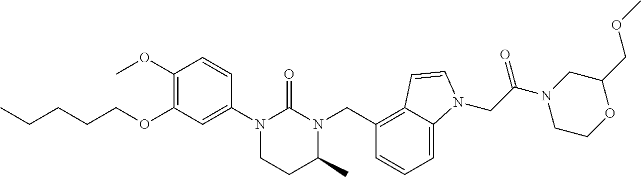 [SMILES: CCCCCOc1cc(N2CC[C@H](C)N(Cc3cccc4c3ccn4CC(=O)N3CCOC(COC)C3)C2=O)ccc1OC] |
| SC0006 | 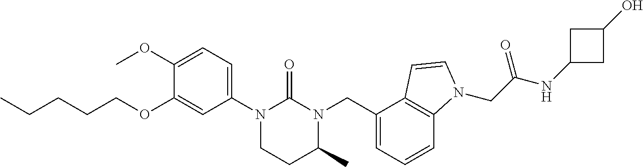 [SMILES: CCCCCOc1cc(N2CC[C@H](C)N(Cc3cccc4c3ccn4CC(=O)NC3CC(O)C3)C2=O)ccc1OC] |
| SC0007 | 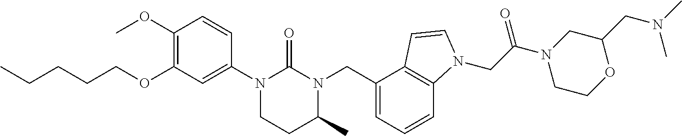 [SMILES: CCCCCOc1cc(N2CC[C@H](C)N(Cc3cccc4c3ccn4CC(=O)N3CCOC(CN(C)C)C3)C2=O)ccc1OC] |
| SC0008 | 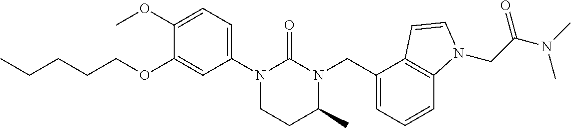 [SMILES: CCCCCOc1cc(N2CC[C@H](C)N(Cc3cccc4c3ccn4CC(=O)N(C)C)C2=O)ccc1OC] |
| SC0009 | 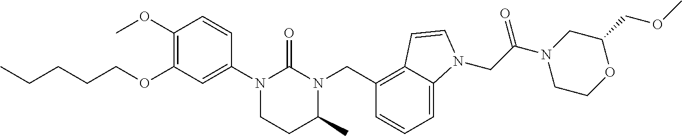 [SMILES: CCCCCOc1cc(N2CC[C@H](C)N(Cc3cccc4c3ccn4CC(=O)N3CCO[C@@H](COC)C3)C2=O)ccc1OC] |
| SC0010 | 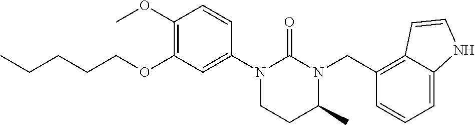 [SMILES: CCCCCOc1cc(N2CC[C@H](C)N(Cc3cccc4[nH]ccc34)C2=O)ccc1OC] |
| CU0623 | 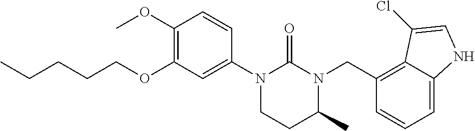 [SMILES: CCCCCOc1cc(N2CC[C@H](C)N(Cc3cccc4[nH]cc(Cl)c34)C2=O)ccc1OC] |
| SC0011 | 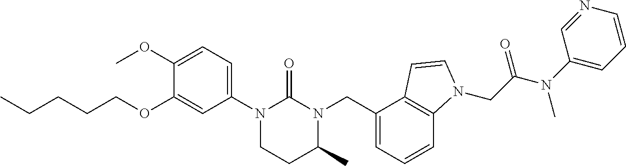 [SMILES: CCCCCOc1cc(N2CC[C@H](C)N(Cc3cccc4c3ccn4CC(=O)N(C)c3cccnc3)C2=O)ccc1OC] |
| SC0012 | 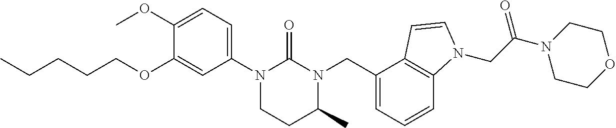 [SMILES: CCCCCOc1cc(N2CC[C@H](C)N(Cc3cccc4c3ccn4CC(=O)N3CCOCC3)C2=O)ccc1OC] |
| SC0013 | 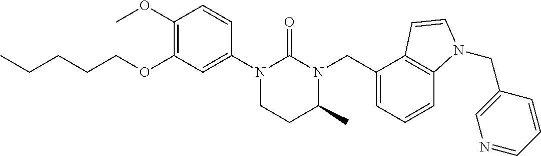 [SMILES: CCCCCOc1cc(N2CC[C@H](C)N(Cc3cccc4c3ccn4Cc3cccnc3)C2=O)ccc1OC] |
| SC0014 | 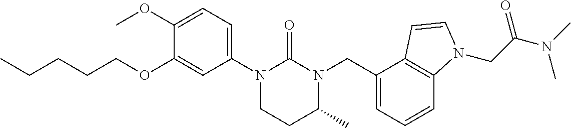 [SMILES: CCCCCOc1cc(N2CC[C@@H](C)N(Cc3cccc4c3ccn4CC(=O)N(C)C)C2=O)ccc1OC] |
| SC0015 | 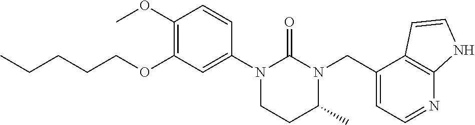 [SMILES: CCCCCOc1cc(N2CC[C@@H](C)N(Cc3ccnc4[nH]ccc34)C2=O)ccc1OC] |
| SC0016 | 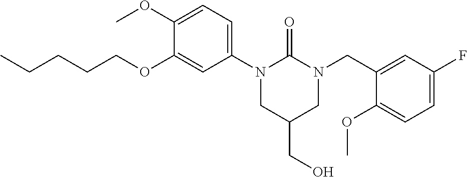 [SMILES: CCCCCOc1cc(N2CC(CO)CN(Cc3cc(F)ccc3OC)C2=O)ccc1OC] |
| SC0017 | 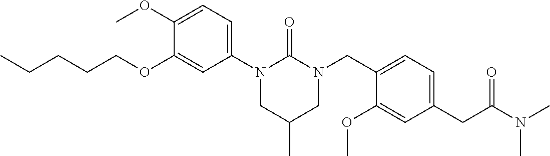 [SMILES: CCCCCOc1cc(N2CC(C)CN(Cc3ccc(CC(=O)N(C)C)cc3OC)C2=O)ccc1OC] |
| SC0018 | 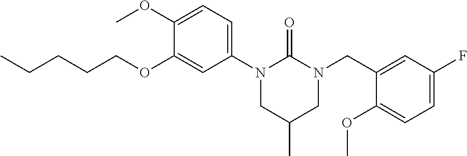 [SMILES: CCCCCOc1cc(N2CC(C)CN(Cc3cc(F)ccc3OC)C2=O)ccc1OC] |
| SC0019 | 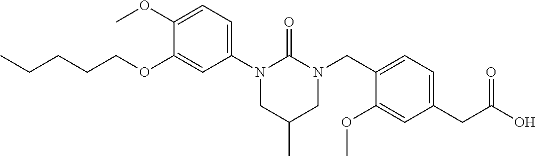 [SMILES: CCCCCOc1cc(N2CC(C)CN(Cc3ccc(CC(=O)O)cc3OC)C2=O)ccc1OC] |
| SC0020 | 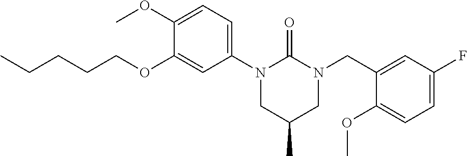 [SMILES: CCCCCOc1cc(N2C[C@H](C)CN(Cc3cc(F)ccc3OC)C2=O)ccc1OC] |
| SC0021 | 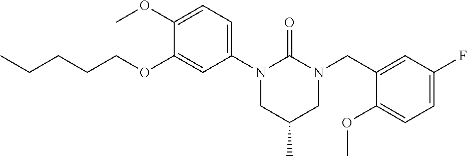 [SMILES: CCCCCOc1cc(N2C[C@@H](C)CN(Cc3cc(F)ccc3OC)C2=O)ccc1OC] |
| SC0022 | 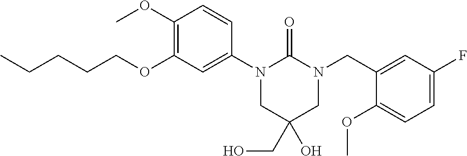 [SMILES: CCCCCOc1cc(N2CC(O)(CO)CN(Cc3cc(F)ccc3OC)C2=O)ccc1OC] |
| SC0023 | 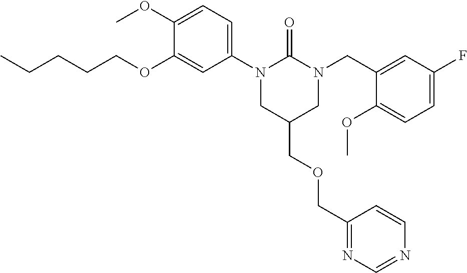 [SMILES: CCCCCOc1cc(N2CC(COCc3ccncn3)CN(Cc3cc(F)ccc3OC)C2=O)ccc1OC] |
| SC0024 | 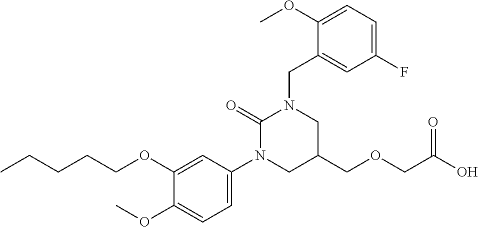 [SMILES: CCCCCOc1cc(N2CC(COCC(=O)O)CN(Cc3cc(F)ccc3OC)C2=O)ccc1OC] |
| SC0025 | 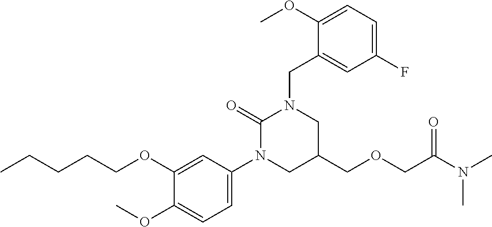 [SMILES: CCCCCOc1cc(N2CC(COCC(=O)N(C)C)CN(Cc3cc(F)ccc3OC)C2=O)ccc1OC] |
| SC0026 | 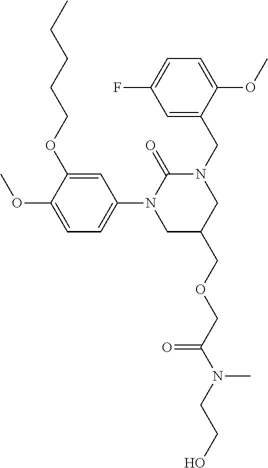 |
| SC0027 | 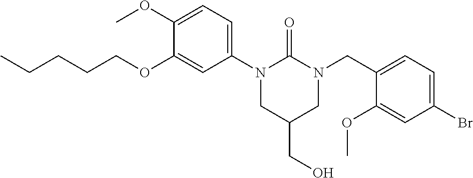 [SMILES: CCCCCOc1cc(N2CC(CO)CN(Cc3ccc(Br)cc3OC)C2=O)ccc1OC] |
| SC0028 | 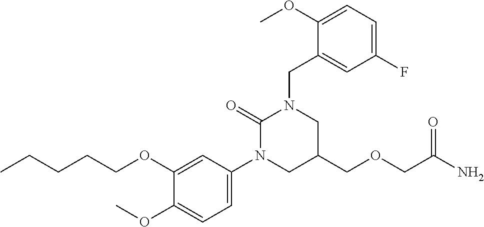 [SMILES: CCCCCOc1cc(N2CC(COCC(N)=O)CN(Cc3cc(F)ccc3OC)C2=O)ccc1OC] |
| SC0029 | 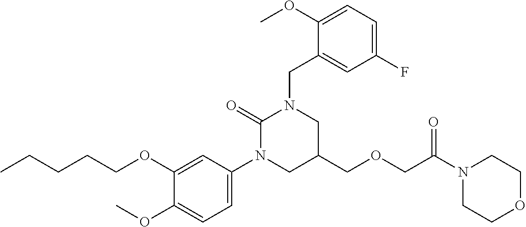 [SMILES: CCCCCOc1cc(N2CC(COCC(=O)N3CCOCC3)CN(Cc3cc(F)ccc3OC)C2=O)ccc1OC] |
| SC0030 | 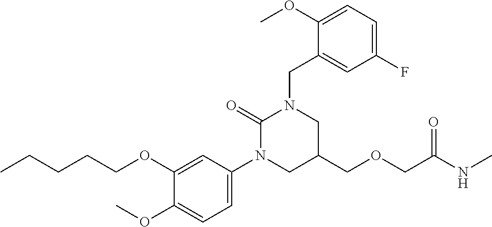 [SMILES: CCCCCOc1cc(N2CC(COCC(=O)NC)CN(Cc3cc(F)ccc3OC)C2=O)ccc1OC] |
| SC0031 | 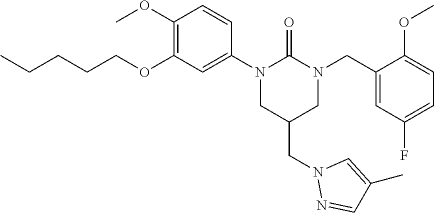 [SMILES: CCCCCOc1cc(N2CC(Cn3cc(C)cn3)CN(Cc3cc(F)ccc3OC)C2=O)ccc1OC] |
| SC0032 | 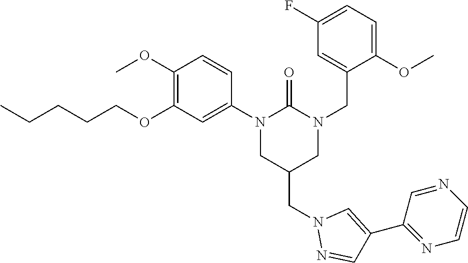 [SMILES: CCCCCOc1cc(N2CC(Cn3cc(-c4cnccn4)cn3)CN(Cc3cc(F)ccc3OC)C2=O)ccc1OC] |
| SC0033 | 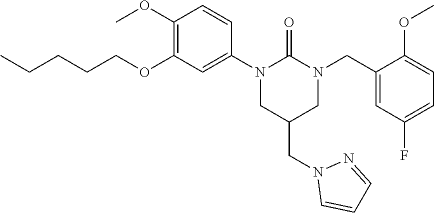 [SMILES: CCCCCOc1cc(N2CC(Cn3cccn3)CN(Cc3cc(F)ccc3OC)C2=O)ccc1OC] |
| SC0034 | 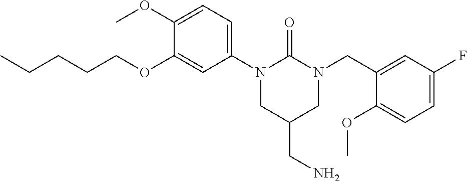 [SMILES: CCCCCOc1cc(N2CC(CN)CN(Cc3cc(F)ccc3OC)C2=O)ccc1OC] |
| SC0035 | 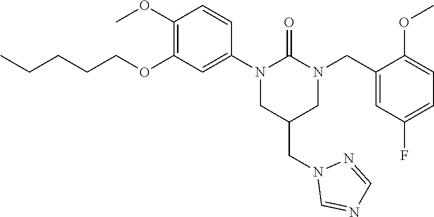 [SMILES: CCCCCOc1cc(N2CC(Cn3cncn3)CN(Cc3cc(F)ccc3OC)C2=O)ccc1OC] |
| SC0036 | 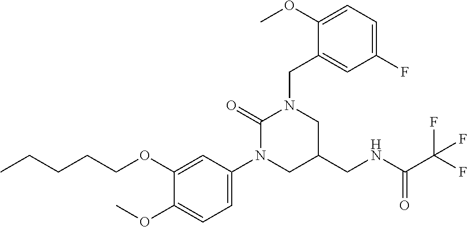 [SMILES: CCCCCOc1cc(N2CC(CNC(=O)C(F)(F)F)CN(Cc3cc(F)ccc3OC)C2=O)ccc1OC] |
| SC0037 | 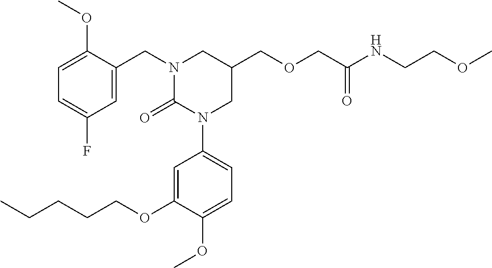 [SMILES: CCCCCOc1cc(N2CC(COCC(=O)NCCOC)CN(Cc3cc(F)ccc3OC)C2=O)ccc1OC] |
| SC0038 | 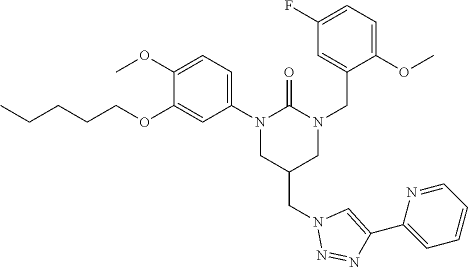 [SMILES: CCCCCOc1cc(N2CC(Cn3cc(-c4ccccn4)nn3)CN(Cc3cc(F)ccc3OC)C2=O)ccc1OC] |
| SC0039 | 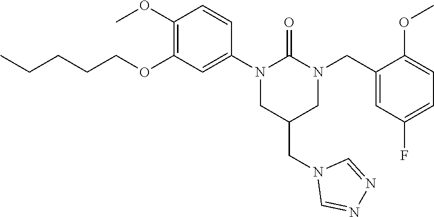 [SMILES: CCCCCOc1cc(N2CC(Cn3cnnc3)CN(Cc3cc(F)ccc3OC)C2=O)ccc1OC] |
| SC0040 | 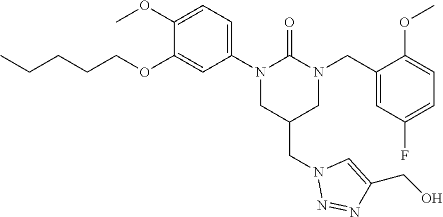 [SMILES: CCCCCOc1cc(N2CC(Cn3cc(CO)nn3)CN(Cc3cc(F)ccc3OC)C2=O)ccc1OC] |
| SC0041 | 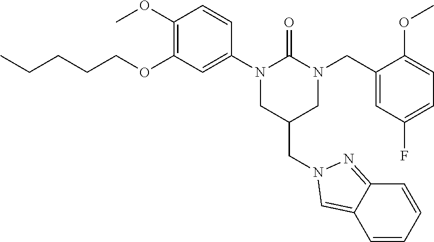 [SMILES: CCCCCOc1cc(N2CC(Cn3cc4ccccc4n3)CN(Cc3cc(F)ccc3OC)C2=O)ccc1OC] |
| SC0042 | 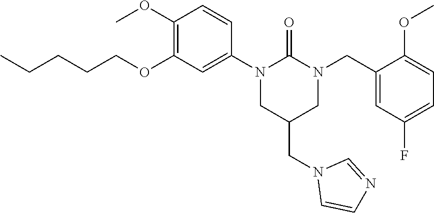 [SMILES: CCCCCOc1cc(N2CC(Cn3ccnc3)CN(Cc3cc(F)ccc3OC)C2=O)ccc1OC] |
| SC0043 | 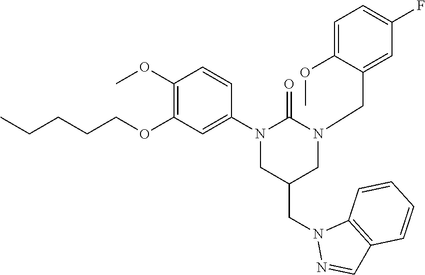 [SMILES: CCCCCOc1cc(N2CC(Cn3ncc4ccccc43)CN(Cc3cc(F)ccc3OC)C2=O)ccc1OC] |
| SC0044 | 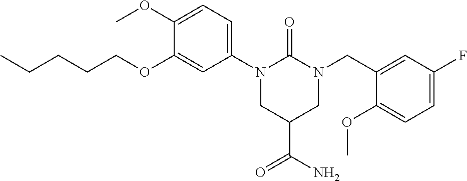 [SMILES: CCCCCOc1cc(N2CC(C(N)=O)CN(Cc3cc(F)ccc3OC)C2=O)ccc1OC] |
| SC0045 | 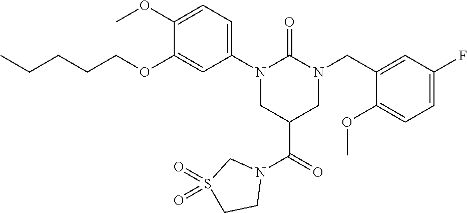 [SMILES: CCCCCOc1cc(N2CC(C(=O)N3CCS(=O)(=O)C3)CN(Cc3cc(F)ccc3OC)C2=O)ccc1OC] |
| SC0046 | 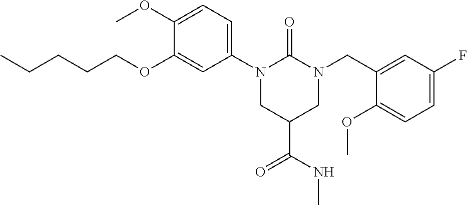 [SMILES: CCCCCOc1cc(N2CC(C(=O)NC)CN(Cc3cc(F)ccc3OC)C2=O)ccc1OC] |
| SC0047 | 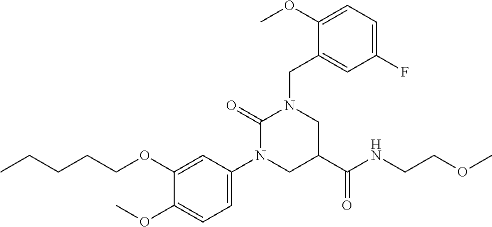 [SMILES: CCCCCOc1cc(N2CC(C(=O)NCCOC)CN(Cc3cc(F)ccc3OC)C2=O)ccc1OC] |
| SC0048 | 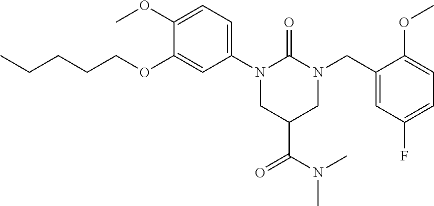 [SMILES: CCCCCOc1cc(N2CC(C(=O)N(C)C)CN(Cc3cc(F)ccc3OC)C2=O)ccc1OC] |
| SC0049 | 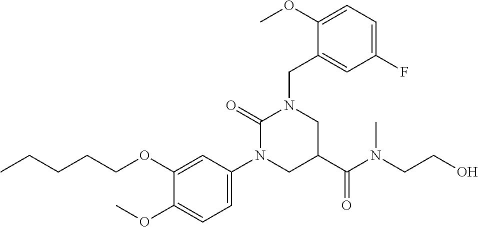 [SMILES: CCCCCOc1cc(N2CC(C(=O)N(C)CCO)CN(Cc3cc(F)ccc3OC)C2=O)ccc1OC] |
| SC0050 | 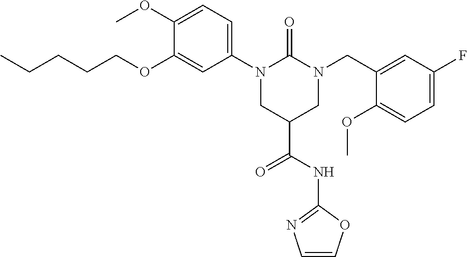 [SMILES: CCCCCOc1cc(N2CC(C(=O)Nc3ncco3)CN(Cc3cc(F)ccc3OC)C2=O)ccc1OC] |
| SC0051 | 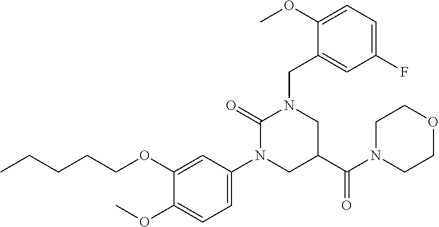 [SMILES: CCCCCOc1cc(N2CC(C(=O)N3CCOCC3)CN(Cc3cc(F)ccc3OC)C2=O)ccc1OC] |
| SC0052 | 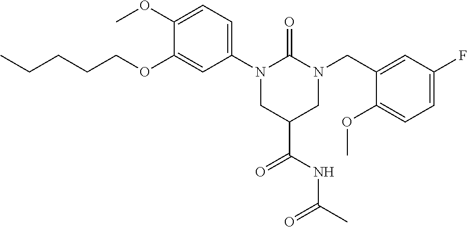 [SMILES: CCCCCOc1cc(N2CC(C(=O)NC(C)=O)CN(Cc3cc(F)ccc3OC)C2=O)ccc1OC] |
| SC0053 | 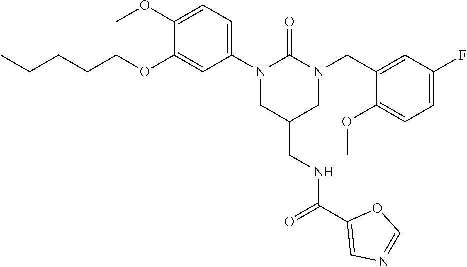 [SMILES: CCCCCOc1cc(N2CC(CNC(=O)c3cnco3)CN(Cc3cc(F)ccc3OC)C2=O)ccc1OC] |
| SC0054 | 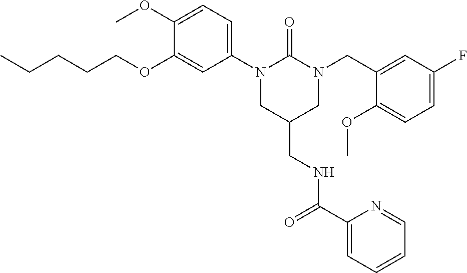 [SMILES: CCCCCOc1cc(N2CC(CNC(=O)c3ccccn3)CN(Cc3cc(F)ccc3OC)C2=O)ccc1OC] |
| SC0055 | 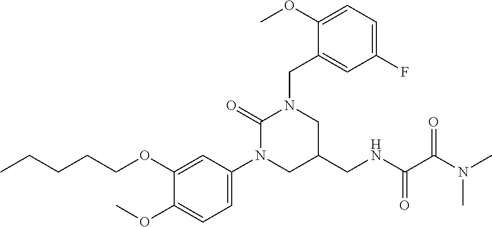 [SMILES: CCCCCOc1cc(N2CC(CNC(=O)C(=O)N(C)C)CN(Cc3cc(F)ccc3OC)C2=O)ccc1OC] |
| SC0056 | 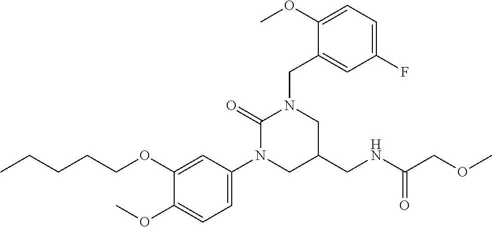 [SMILES: CCCCCOc1cc(N2CC(CNC(=O)COC)CN(Cc3cc(F)ccc3OC)C2=O)ccc1OC] |
| SC0057 | 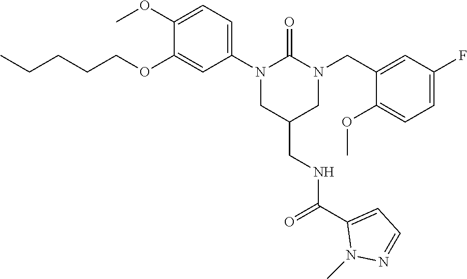 [SMILES: CCCCCOc1cc(N2CC(CNC(=O)c3ccnn3C)CN(Cc3cc(F)ccc3OC)C2=O)ccc1OC] |
| SC0058 | 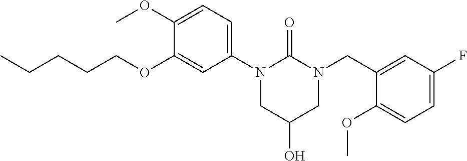 [SMILES: CCCCCOc1cc(N2CC(O)CN(Cc3cc(F)ccc3OC)C2=O)ccc1OC] |
| SC0059 | 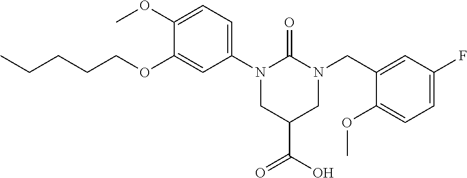 [SMILES: CCCCCOc1cc(N2CC(C(=O)O)CN(Cc3cc(F)ccc3OC)C2=O)ccc1OC] |
| SC0060 | 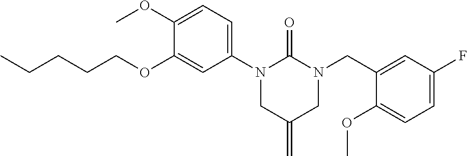 [SMILES: C=C1CN(Cc2cc(F)ccc2OC)C(=O)N(c2ccc(OC)c(OCCCCC)c2)C1] |
| SC0061 | 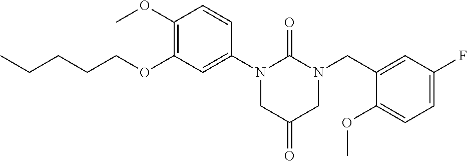 [SMILES: CCCCCOc1cc(N2CC(=O)CN(Cc3cc(F)ccc3OC)C2=O)ccc1OC] |
| SC0062 | 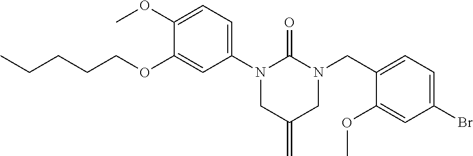 [SMILES: C=C1CN(Cc2ccc(Br)cc2OC)C(=O)N(c2ccc(OC)c(OCCCCC)c2)C1] |
| SC0063 | 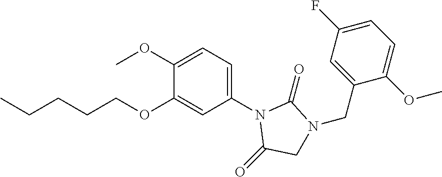 [SMILES: CCCCCOc1cc(N2C(=O)CN(Cc3cc(F)ccc3OC)C2=O)ccc1OC] |
| SC0064 | 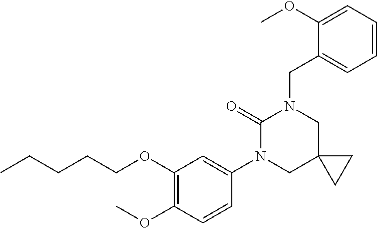 [SMILES: CCCCCOc1cc(N2CC3(CC3)CN(Cc3ccccc3OC)C2=O)ccc1OC] |
| SC0065 | 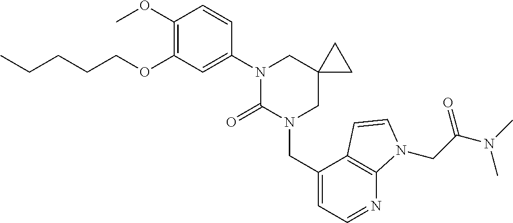 [SMILES: CCCCCOc1cc(N2CC3(CC3)CN(Cc3ccnc4c3ccn4CC(=O)N(C)C)C2=O)ccc1OC] |
| SC0066 | 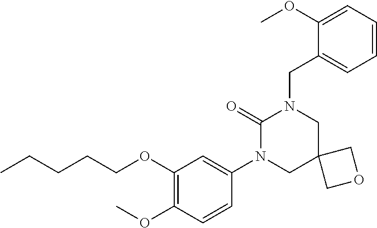 [SMILES: CCCCCOc1cc(N2CC3(COC3)CN(Cc3ccccc3OC)C2=O)ccc1OC] |
| SC0067 | 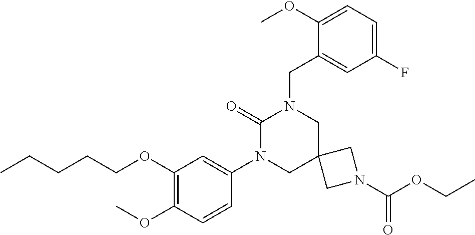 [SMILES: CCCCCOc1cc(N2CC3(CN(C(=O)OCC)C3)CN(Cc3cc(F)ccc3OC)C2=O)ccc1OC] |
| SC0068 | 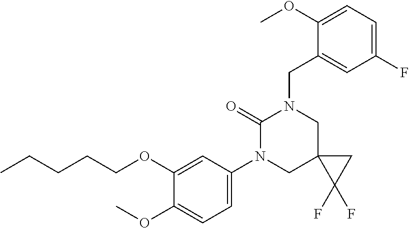 [SMILES: CCCCCOc1cc(N2CC3(CN(Cc4cc(F)ccc4OC)C2=O)CC3(F)F)ccc1OC] |
| SC0069 | 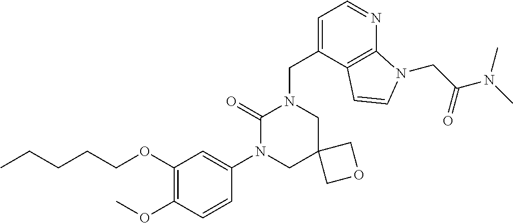 [SMILES: CCCCCOc1cc(N2CC3(COC3)CN(Cc3ccnc4c3ccn4CC(=O)N(C)C)C2=O)ccc1OC] |
| SC0070 | 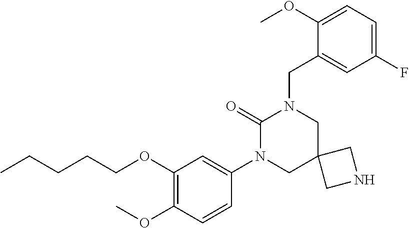 [SMILES: CCCCCOc1cc(N2CC3(CNC3)CN(Cc3cc(F)ccc3OC)C2=O)ccc1OC] |
| SC0071 | 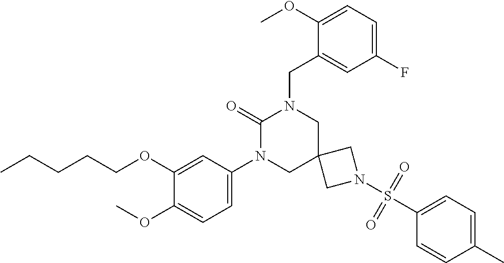 [SMILES: CCCCCOc1cc(N2CC3(CN(Cc4cc(F)ccc4OC)C2=O)CN(S(=O)(=O)c2ccc(C)cc2)C3)ccc1OC] |
| SC0072 | 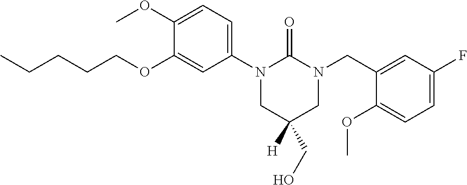 [SMILES: CCCCCOc1cc(N2C[C@@H](CO)CN(Cc3cc(F)ccc3OC)C2=O)ccc1OC] |
| SC0100 | 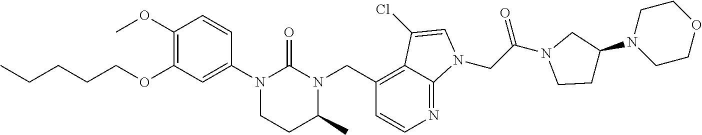 [SMILES: CCCCCOc1cc(N2CC[C@H](C)N(Cc3ccnc4c3c(Cl)cn4CC(=O)N3CC[C@H](N4CCOCC4)C3)C2=O)ccc1OC] |
| SC0101 | 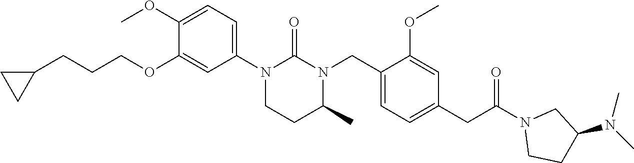 [SMILES: COc1cc(CC(=O)N2CC[C@H](N(C)C)C2)ccc1CN1C(=O)N(c2ccc(OC)c(OCCCC3CC3)c2)CC[C@@H]1C] |
| SC0102 | 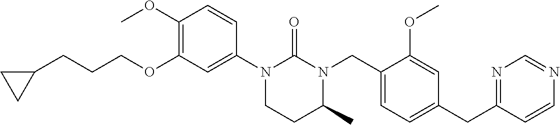 [SMILES: COc1cc(Cc2ccncn2)ccc1CN1C(=O)N(c2ccc(OC)c(OCCCC3CC3)c2)CC[C@@H]1C] |
| SC0103 | 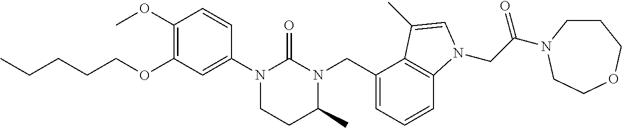 [SMILES: CCCCCOc1cc(N2CC[C@H](C)N(Cc3cccc4c3c(C)cn4CC(=O)N3CCCOCC3)C2=O)ccc1OC] |
| SC0104 | 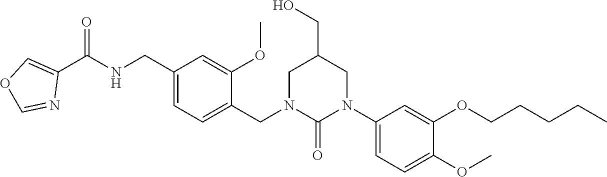 [SMILES: CCCCCOc1cc(N2CC(CO)CN(Cc3ccc(CNC(=O)c4cocn4)cc3OC)C2=O)ccc1OC] |
| SC0105 | 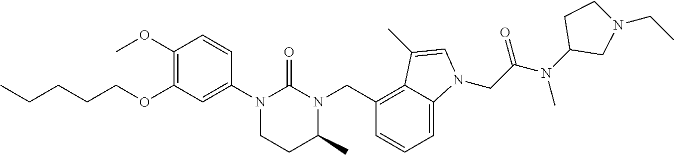 [SMILES: CCCCCOc1cc(N2CC[C@H](C)N(Cc3cccc4c3c(C)cn4CC(=O)N(C)C3CCN(CC)C3)C2=O)ccc1OC] |
| SC0106 | 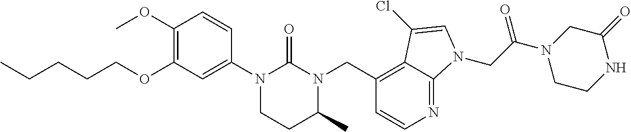 [SMILES: CCCCCOc1cc(N2CC[C@H](C)N(Cc3ccnc4c3c(Cl)cn4CC(=O)N3CCNC(=O)C3)C2=O)ccc1OC] |
| SC0107 | 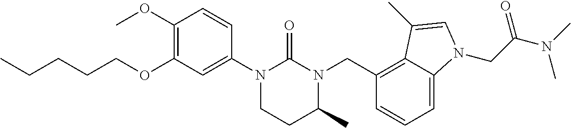 [SMILES: CCCCCOc1cc(N2CC[C@H](C)N(Cc3cccc4c3c(C)cn4CC(=O)N(C)C)C2=O)ccc1OC] |
| SC0108 | 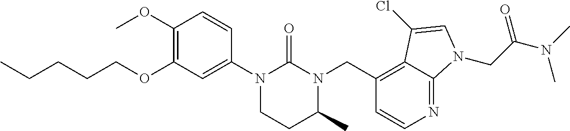 [SMILES: CCCCCOc1cc(N2CC[C@H](C)N(Cc3ccnc4c3c(Cl)cn4CC(=O)N(C)C)C2=O)ccc1OC] |
| SC0109 | 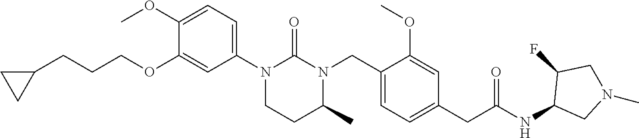 [SMILES: COc1cc(CC(=O)N[C@@H]2CN(C)C[C@@H]2F)ccc1CN1C(=O)N(c2ccc(OC)c(OCCCC3CC3)c2)CC[C@@H]1C] |
| SC0110 | 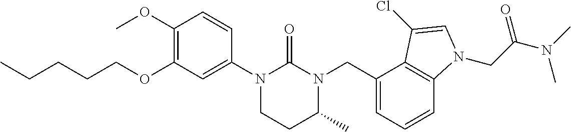 [SMILES: CCCCCOc1cc(N2CC[C@@H](C)N(Cc3cccc4c3c(Cl)cn4CC(=O)N(C)C)C2=O)ccc1OC] |
| SC0111 | 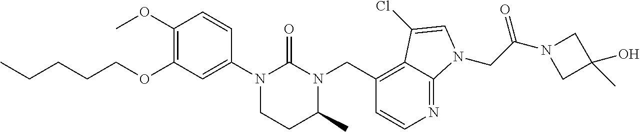 [SMILES: CCCCCOc1cc(N2CC[C@H](C)N(Cc3ccnc4c3c(Cl)cn4CC(=O)N3CC(C)(O)C3)C2=O)ccc1OC] |
| SC0112 | 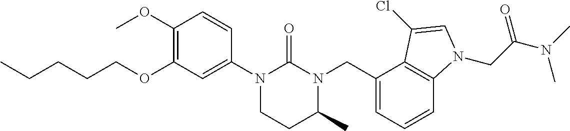 [SMILES: CCCCCOc1cc(N2CC[C@H](C)N(Cc3cccc4c3c(Cl)cn4CC(=O)N(C)C)C2=O)ccc1OC] |
| SC0113 | 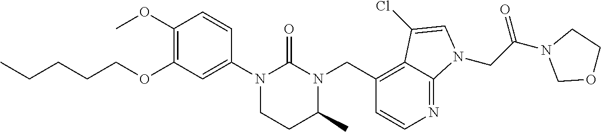 [SMILES: CCCCCOc1cc(N2CC[C@H](C)N(Cc3ccnc4c3c(Cl)cn4CC(=O)N3CCOC3)C2=O)ccc1OC] |
| SC0114 | 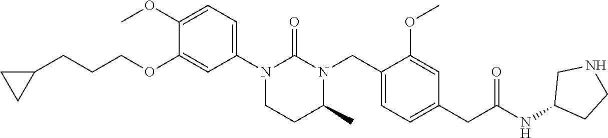 [SMILES: COc1cc(CC(=O)N[C@H]2CCNC2)ccc1CN1C(=O)N(c2ccc(OC)c(OCCCC3CC3)c2)CC[C@@H]1C] |
| SC0115 | 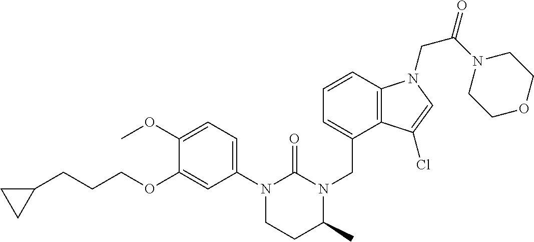 [SMILES: COc1ccc(N2CC[C@H](C)N(Cc3cccc4c3c(Cl)cn4CC(=O)N3CCOCC3)C2=O)cc1OCCCC1CC1] |
| SC0116 | 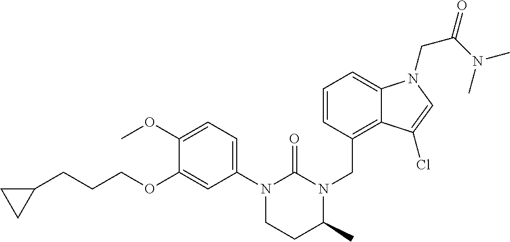 [SMILES: COc1ccc(N2CC[C@H](C)N(Cc3cccc4c3c(Cl)cn4CC(=O)N(C)C)C2=O)cc1OCCCC1CC1] |
| SC0117 | 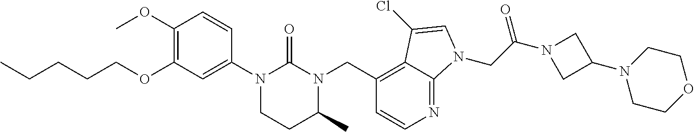 [SMILES: CCCCCOc1cc(N2CC[C@H](C)N(Cc3ccnc4c3c(Cl)cn4CC(=O)N3CC(N4CCOCC4)C3)C2=O)ccc1OC] |
| SC0118 | 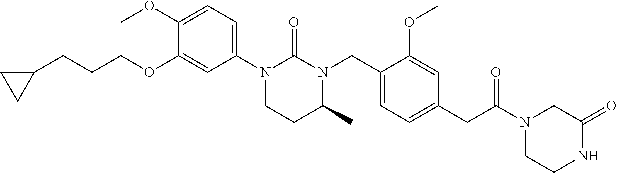 [SMILES: COc1cc(CC(=O)N2CCNC(=O)C2)ccc1CN1C(=O)N(c2ccc(OC)c(OCCCC3CC3)c2)CC[C@@H]1C] |
| SC0119 | 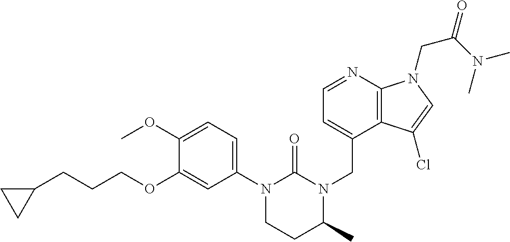 [SMILES: COc1ccc(N2CC[C@H](C)N(Cc3ccnc4c3c(Cl)cn4CC(=O)N(C)C)C2=O)cc1OCCCC1CC1] |
| SC0120 | 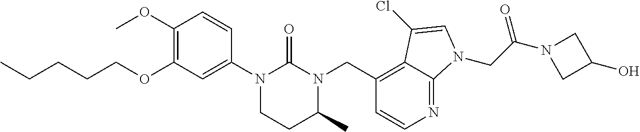 [SMILES: CCCCCOc1cc(N2CC[C@H](C)N(Cc3ccnc4c3c(Cl)cn4CC(=O)N3CC(O)C3)C2=O)ccc1OC] |
| SC0121 | 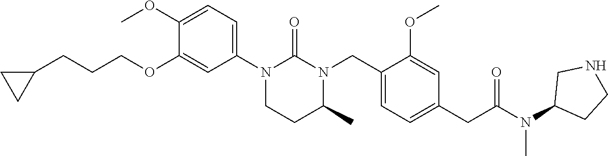 [SMILES: COc1cc(CC(=O)N(C)[C@@H]2CCNC2)ccc1CN1C(=O)N(c2ccc(OC)c(OCCCC3CC3)c2)CC[C@@H]1C] |
| SC0122 | 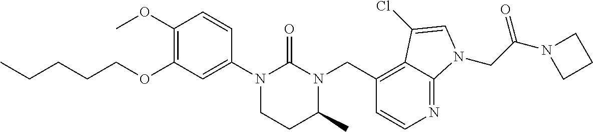 [SMILES: CCCCCOc1cc(N2CC[C@H](C)N(Cc3ccnc4c3c(Cl)cn4CC(=O)N3CCC3)C2=O)ccc1OC] |
| SC0123 | 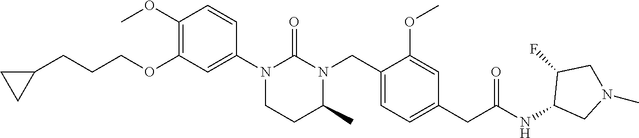 [SMILES: COc1cc(CC(=O)N[C@H]2CN(C)C[C@H]2F)ccc1CN1C(=O)N(c2ccc(OC)c(OCCCC3CC3)c2)CC[C@@H]1C] |
| SC0124 | 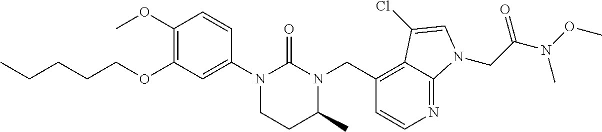 [SMILES: CCCCCOc1cc(N2CC[C@H](C)N(Cc3ccnc4c3c(Cl)cn4CC(=O)N(C)OC)C2=O)ccc1OC] |
| SC0125 | 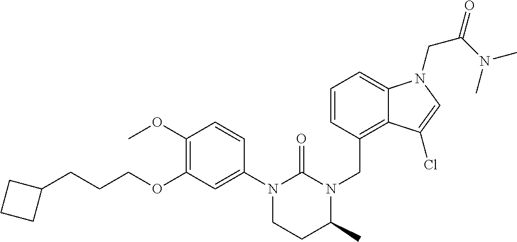 [SMILES: COc1ccc(N2CC[C@H](C)N(Cc3cccc4c3c(Cl)cn4CC(=O)N(C)C)C2=O)cc1OCCCC1CCC1] |
| SC0126 | 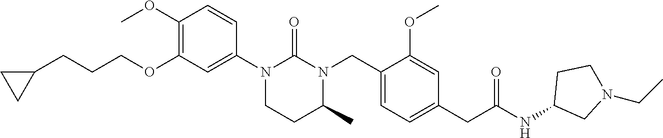 [SMILES: CCN1CC[C@@H](NC(=O)Cc2ccc(CN3C(=O)N(c4ccc(OC)c(OCCCC5CC5)c4)CC[C@@H]3C)c(OC)c2)C1] |
| SC0127 | 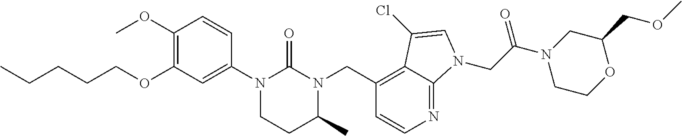 [SMILES: CCCCCOc1cc(N2CC[C@H](C)N(Cc3ccnc4c3c(Cl)cn4CC(=O)N3CCO[C@H](COC)C3)C2=O)ccc1OC] |
| SC0128 | 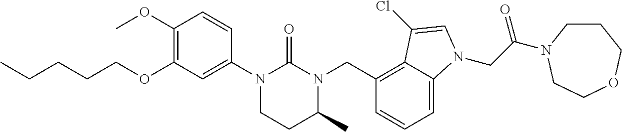 [SMILES: CCCCCOc1cc(N2CC[C@H](C)N(Cc3cccc4c3c(Cl)cn4CC(=O)N3CCCOCC3)C2=O)ccc1OC] |
| SC0129 | 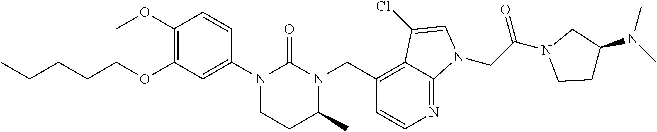 [SMILES: CCCCCOc1cc(N2CC[C@H](C)N(Cc3ccnc4c3c(Cl)cn4CC(=O)N3CC[C@H](N(C)C)C3)C2=O)ccc1OC] |
| SC0130 | 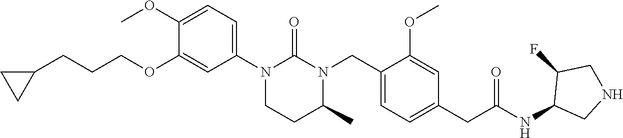 [SMILES: COc1cc(CC(=O)N[C@@H]2CNC[C@@H]2F)ccc1CN1C(=O)N(c2ccc(OC)c(OCCCC3CC3)c2)CC[C@@H]1C] |
| SC0131 | 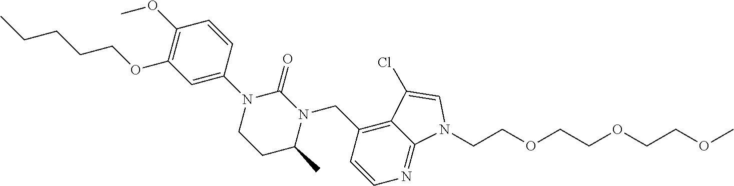 [SMILES: CCCCCOc1cc(N2CC[C@H](C)N(Cc3ccnc4c3c(Cl)cn4CCOCCOCCOC)C2=O)ccc1OC] |
| SC0132 | 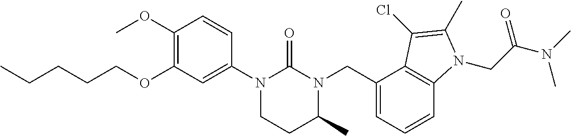 [SMILES: CCCCCOc1cc(N2CC[C@H](C)N(Cc3cccc4c3c(Cl)c(C)n4CC(=O)N(C)C)C2=O)ccc1OC] |
| SC0133 | 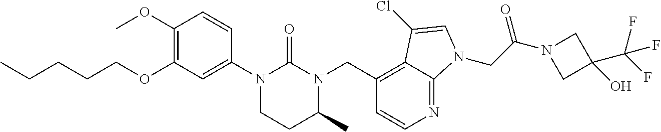 [SMILES: CCCCCOc1cc(N2CC[C@H](C)N(Cc3ccnc4c3c(Cl)cn4CC(=O)N3CC(O)(C(F)(F)F)C3)C2=O)ccc1OC] |
| SC0134 | 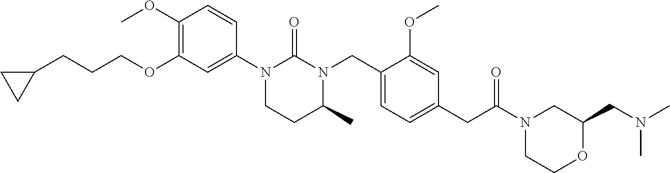 [SMILES: COc1cc(CC(=O)N2CCO[C@H](CN(C)C)C2)ccc1CN1C(=O)N(c2ccc(OC)c(OCCCC3CC3)c2)CC[C@@H]1C] |
| SC0135 | 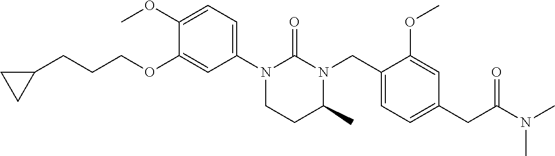 [SMILES: COc1cc(CC(=O)N(C)C)ccc1CN1C(=O)N(c2ccc(OC)c(OCCCC3CC3)c2)CC[C@@H]1C] |
| SC0136 | 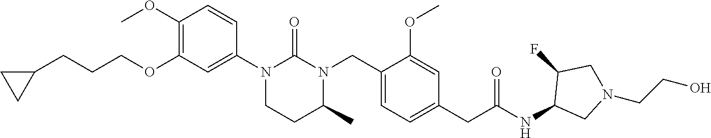 [SMILES: COc1cc(CC(=O)N[C@@H]2CN(CCO)C[C@@H]2F)ccc1CN1C(=O)N(c2ccc(OC)c(OCCCC3CC3)c2)CC[C@@H]1C] |
| SC0137 | 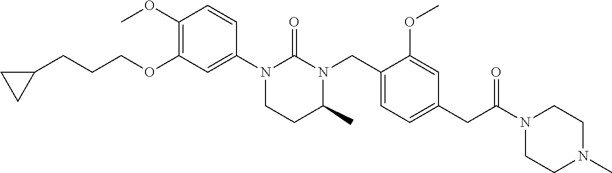 [SMILES: COc1cc(CC(=O)N2CCN(C)CC2)ccc1CN1C(=O)N(c2ccc(OC)c(OCCCC3CC3)c2)CC[C@@H]1C] |
| SC0138 | 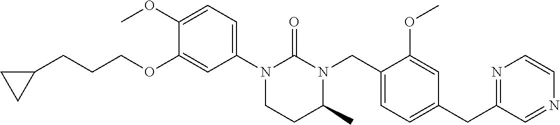 [SMILES: COc1cc(Cc2cnccn2)ccc1CN1C(=O)N(c2ccc(OC)c(OCCCC3CC3)c2)CC[C@@H]1C] |
| SC0139 | 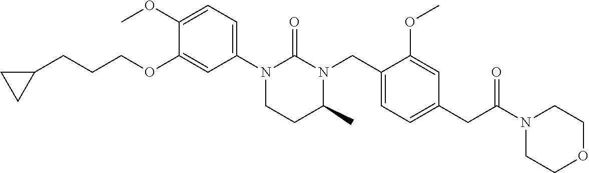 [SMILES: COc1cc(CC(=O)N2CCOCC2)ccc1CN1C(=O)N(c2ccc(OC)c(OCCCC3CC3)c2)CC[C@@H]1C] |
| SC0140 | 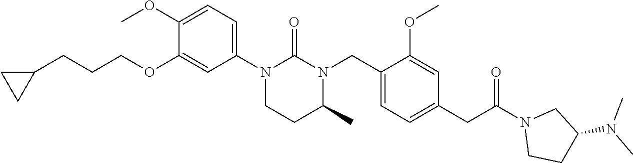 [SMILES: COc1cc(CC(=O)N2CC[C@@H](N(C)C)C2)ccc1CN1C(=O)N(c2ccc(OC)c(OCCCC3CC3)c2)CC[C@@H]1C] |
| SC0141 | 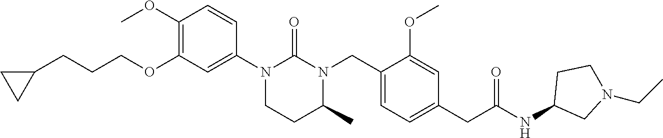 [SMILES: CCN1CC[C@H](NC(=O)Cc2ccc(CN3C(=O)N(c4ccc(OC)c(OCCCC5CC5)c4)CC[C@@H]3C)c(OC)c2)C1] |
| SC0142 | 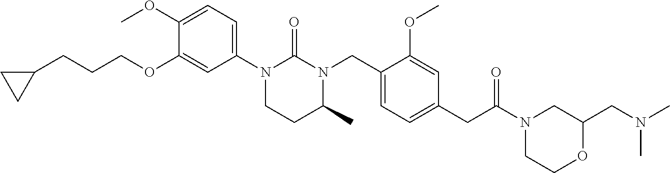 [SMILES: COc1cc(CC(=O)N2CCOC(CN(C)C)C2)ccc1CN1C(=O)N(c2ccc(OC)c(OCCCC3CC3)c2)CC[C@@H]1C] |
| SC0143 | 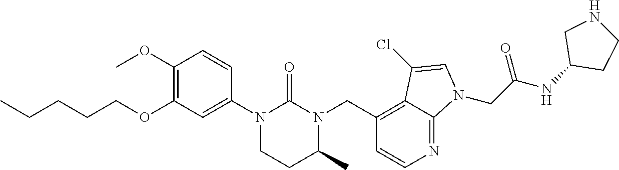 [SMILES: CCCCCOc1cc(N2CC[C@H](C)N(Cc3ccnc4c3c(Cl)cn4CC(=O)N[C@H]3CCNC3)C2=O)ccc1OC] |
| SC0144 | 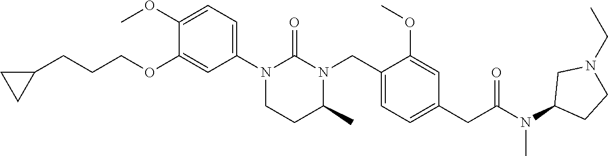 [SMILES: CCN1CC[C@@H](N(C)C(=O)Cc2ccc(CN3C(=O)N(c4ccc(OC)c(OCCCC5CC5)c4)CC[C@@H]3C)c(OC)c2)C1] |
| SC0145 | 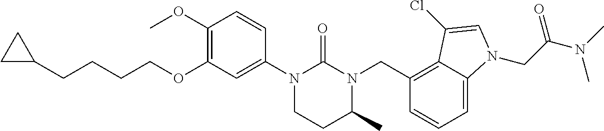 [SMILES: COc1ccc(N2CC[C@H](C)N(Cc3cccc4c3c(Cl)cn4CC(=O)N(C)C)C2=O)cc1OCCCCC1CC1] |
| SC0146 | 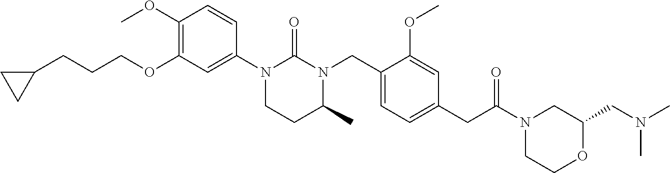 [SMILES: COc1cc(CC(=O)N2CCO[C@@H](CN(C)C)C2)ccc1CN1C(=O)N(c2ccc(OC)c(OCCCC3CC3)c2)CC[C@@H]1C] |
| SC0147 | 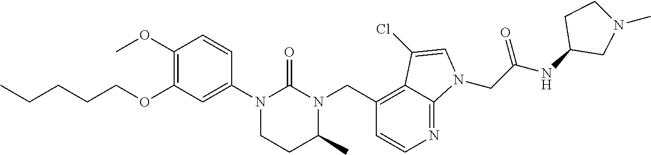 [SMILES: CCCCCOc1cc(N2CC[C@H](C)N(Cc3ccnc4c3c(Cl)cn4CC(=O)N[C@H]3CCN(C)C3)C2=O)ccc1OC] |
| SC0148 | 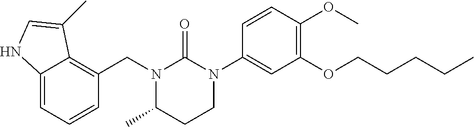 [SMILES: CCCCCOc1cc(N2CC[C@H](C)N(Cc3cccc4[nH]cc(C)c34)C2=O)ccc1OC] |
| SC0149 | 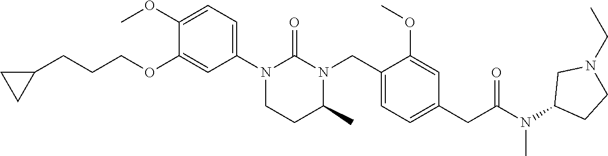 [SMILES: CCN1CC[C@H](N(C)C(=O)Cc2ccc(CN3C(=O)N(c4ccc(OC)c(OCCCC5CC5)c4)CC[C@@H]3C)c(OC)c2)C1] |
| SC0150 | 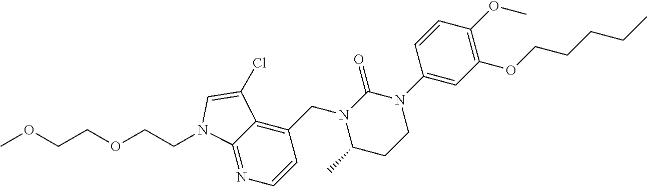 [SMILES: CCCCCOc1cc(N2CC[C@H](C)N(Cc3ccnc4c3c(Cl)cn4CCOCCOC)C2=O)ccc1OC] |
| SC0151 | 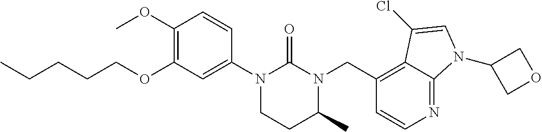 [SMILES: CCCCCOc1cc(N2CC[C@H](C)N(Cc3ccnc4c3c(Cl)cn4C3COC3)C2=O)ccc1OC] |
| SC0152 | 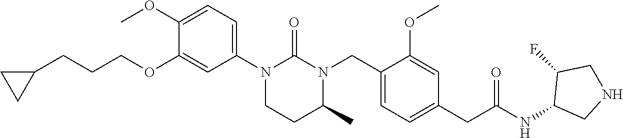 [SMILES: COc1cc(CC(=O)N[C@H]2CNC[C@H]2F)ccc1CN1C(=O)N(c2ccc(OC)c(OCCCC3CC3)c2)CC[C@@H]1C] |
| SC0153 | 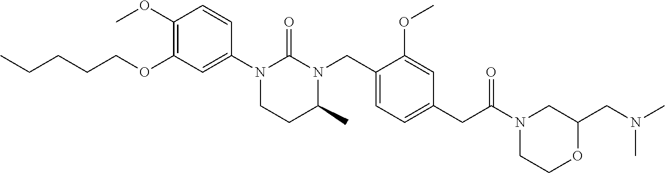 [SMILES: CCCCCOc1cc(N2CC[C@H](C)N(Cc3ccc(CC(=O)N4CCOC(CN(C)C)C4)cc3OC)C2=O)ccc1OC] |
| SC0154 | 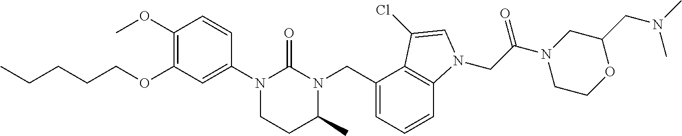 [SMILES: CCCCCOc1cc(N2CC[C@H](C)N(Cc3cccc4c3c(Cl)cn4CC(=O)N3CCOC(CN(C)C)C3)C2=O)ccc1OC] |
| SC0155 | 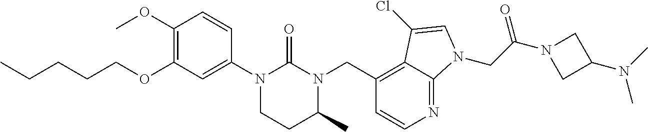 [SMILES: CCCCCOc1cc(N2CC[C@H](C)N(Cc3ccnc4c3c(Cl)cn4CC(=O)N3CC(N(C)C)C3)C2=O)ccc1OC] |
| SC0156 | 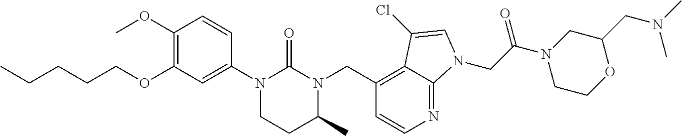 [SMILES: CCCCCOc1cc(N2CC[C@H](C)N(Cc3ccnc4c3c(Cl)cn4CC(=O)N3CCOC(CN(C)C)C3)C2=O)ccc1OC] |
| SC0157 | 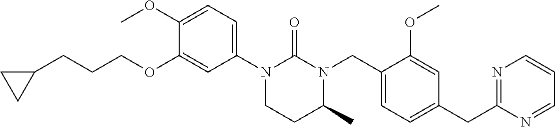 [SMILES: COc1cc(Cc2ncccn2)ccc1CN1C(=O)N(c2ccc(OC)c(OCCCC3CC3)c2)CC[C@@H]1C] |
| SC0158 | 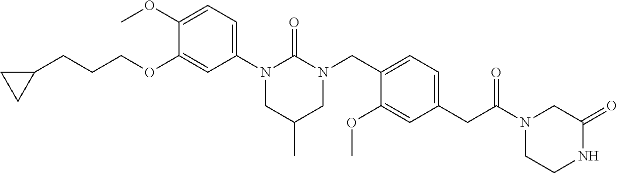 [SMILES: COc1cc(CC(=O)N2CCNC(=O)C2)ccc1CN1CC(C)CN(c2ccc(OC)c(OCCCC3CC3)c2)C1=O] |
| SC0159 | 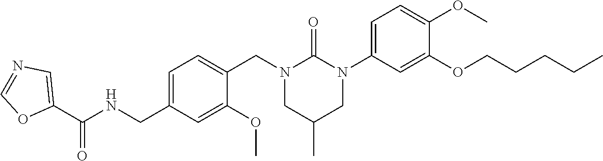 [SMILES: CCCCCOc1cc(N2CC(C)CN(Cc3ccc(CNC(=O)c4cnco4)cc3OC)C2=O)ccc1OC] |
| SC0160 | 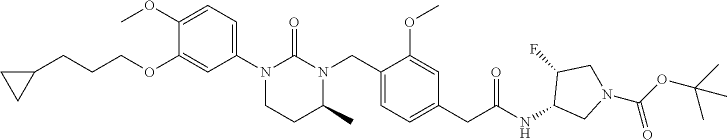 [SMILES: COc1cc(CC(=O)N[C@H]2CN(C(=O)OC(C)(C)C)C[C@H]2F)ccc1CN1C(=O)N(c2ccc(OC)c(OCCCC3CC3)c2)CC[C@@H]1C] |
| SC0161 | 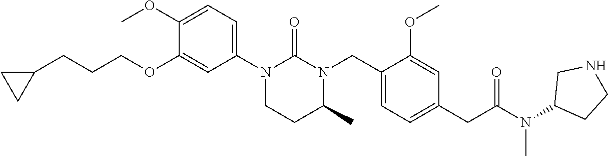 |
| SC0162 | 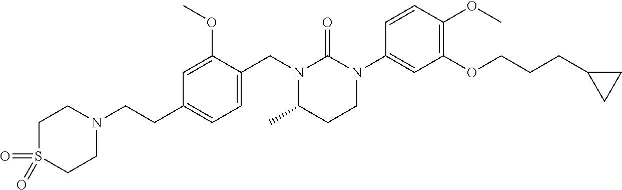 [SMILES: COc1cc(CCN2CCS(=O)(=O)CC2)ccc1CN1C(=O)N(c2ccc(OC)c(OCCCC3CC3)c2)CC[C@@H]1C] |
| SC0163 | 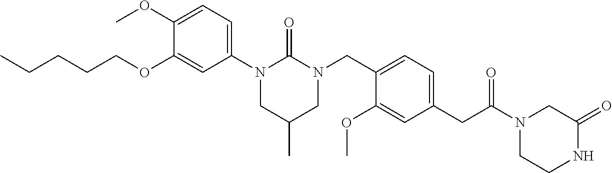 [SMILES: CCCCCOc1cc(N2CC(C)CN(Cc3ccc(CC(=O)N4CCNC(=O)C4)cc3OC)C2=O)ccc1OC] |
| SC0164 | 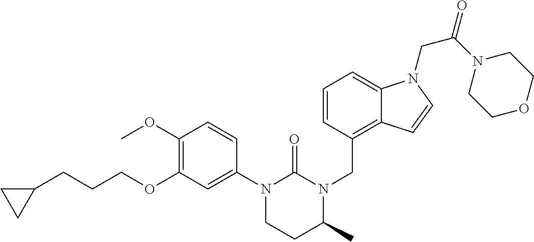 [SMILES: COc1ccc(N2CC[C@H](C)N(Cc3cccc4c3ccn4CC(=O)N3CCOCC3)C2=O)cc1OCCCC1CC1] |
| SC0165 | 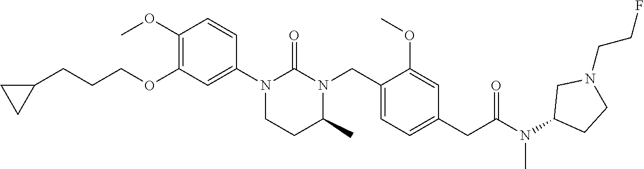 [SMILES: COc1cc(CC(=O)N(C)[C@H]2CCN(CCF)C2)ccc1CN1C(=O)N(c2ccc(OC)c(OCCCC3CC3)c2)CC[C@@H]1C] |
| SC0166 | 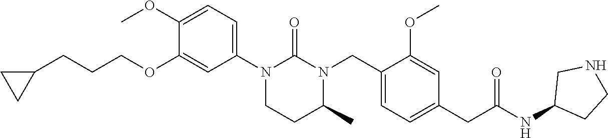 [SMILES: COc1cc(CC(=O)N[C@@H]2CCNC2)ccc1CN1C(=O)N(c2ccc(OC)c(OCCCC3CC3)c2)CC[C@@H]1C] |
| SC0167 | 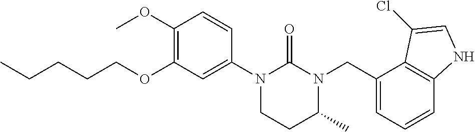 [SMILES: CCCCCOc1cc(N2CC[C@@H](C)N(Cc3cccc4[nH]cc(Cl)c34)C2=O)ccc1OC] |
| SC0168 | 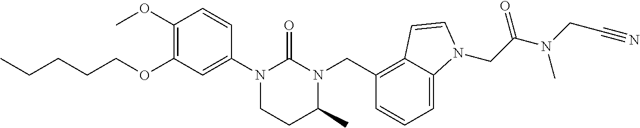 [SMILES: CCCCCOc1cc(N2CC[C@H](C)N(Cc3cccc4c3ccn4CC(=O)N(C)CC#N)C2=O)ccc1OC] |
| SC0169 | 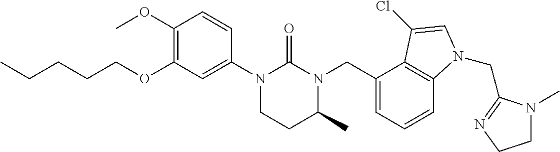 [SMILES: CCCCCOc1cc(N2CC[C@H](C)N(Cc3cccc4c3c(Cl)cn4CC3=NCCN3C)C2=O)ccc1OC] |
| SC0170 | 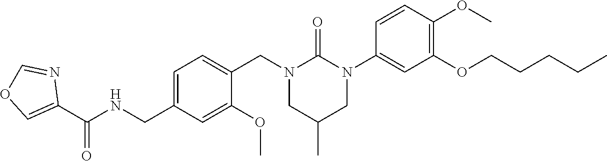 [SMILES: CCCCCOc1cc(N2CC(C)CN(Cc3ccc(CNC(=O)c4cocn4)cc3OC)C2=O)ccc1OC] |
| SC0171 | 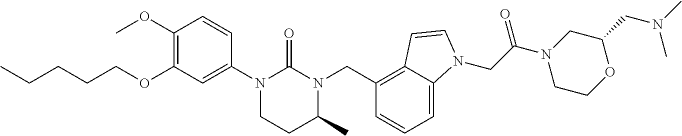 [SMILES: CCCCCOc1cc(N2CC[C@H](C)N(Cc3cccc4c3ccn4CC(=O)N3CCO[C@@H](CN(C)C)C3)C2=O)ccc1OC] |
| SC0172 | 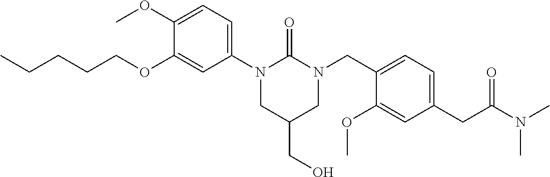 [SMILES: CCCCCOc1cc(N2CC(CO)CN(Cc3ccc(CC(=O)N(C)C)cc3OC)C2=O)ccc1OC] |
| SC0173 | 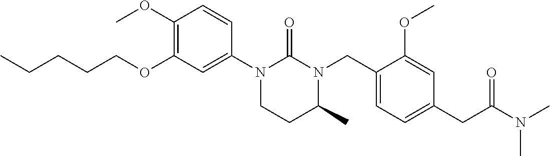 [SMILES: CCCCCOc1cc(N2CC[C@H](C)N(Cc3ccc(CC(=O)N(C)C)cc3OC)C2=O)ccc1OC] |
| SC0174 | 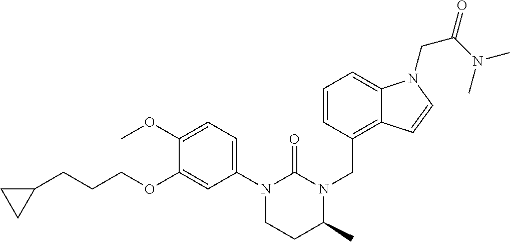 [SMILES: COc1ccc(N2CC[C@H](C)N(Cc3cccc4c3ccn4CC(=O)N(C)C)C2=O)cc1OCCCC1CC1] |
| SC0175 | 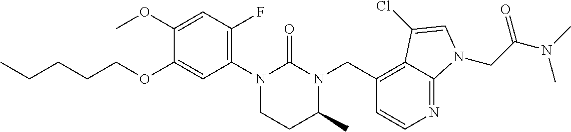 [SMILES: CCCCCOc1cc(N2CC[C@H](C)N(Cc3ccnc4c3c(Cl)cn4CC(=O)N(C)C)C2=O)c(F)cc1OC] |
| SC0176 | 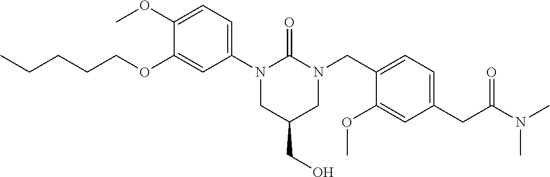 [SMILES: CCCCCOc1cc(N2C[C@H](CO)CN(Cc3ccc(CC(=O)N(C)C)cc3OC)C2=O)ccc1OC] |
| SC0177 | 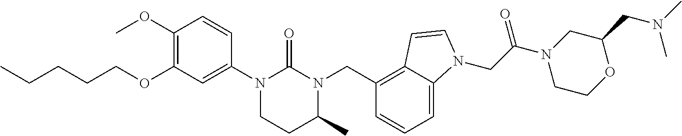 [SMILES: CCCCCOc1cc(N2CC[C@H](C)N(Cc3cccc4c3ccn4CC(=O)N3CCO[C@H](CN(C)C)C3)C2=O)ccc1OC] |
| SC0178 | 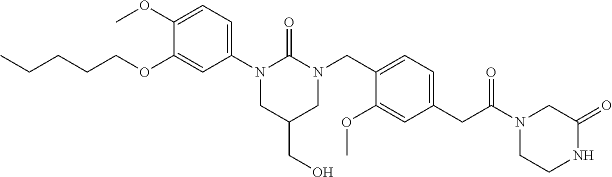 [SMILES: CCCCCOc1cc(N2CC(CO)CN(Cc3ccc(CC(=O)N4CCNC(=O)C4)cc3OC)C2=O)ccc1OC] |
| SC0179 | 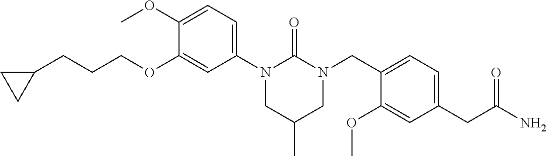 [SMILES: COc1cc(CC(N)=O)ccc1CN1CC(C)CN(c2ccc(OC)c(OCCCC3CC3)c2)C1=O] |
| SC0180 | 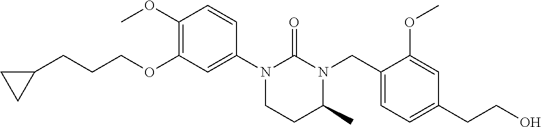 [SMILES: COc1cc(CCO)ccc1CN1C(=O)N(c2ccc(OC)c(OCCCC3CC3)c2)CC[C@@H]1C] |
| SC0181 | 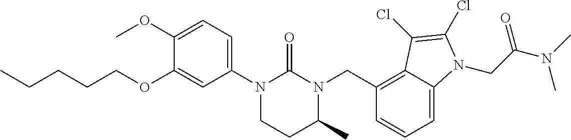 [SMILES: CCCCCOc1cc(N2CC[C@H](C)N(Cc3cccc4c3c(Cl)c(Cl)n4CC(=O)N(C)C)C2=O)ccc1OC] |
| SC0182 | 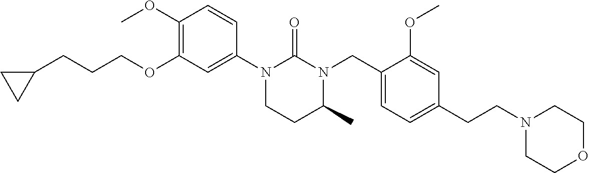 [SMILES: COc1cc(CCN2CCOCC2)ccc1CN1C(=O)N(c2ccc(OC)c(OCCCC3CC3)c2)CC[C@@H]1C] |
| SC0183 | 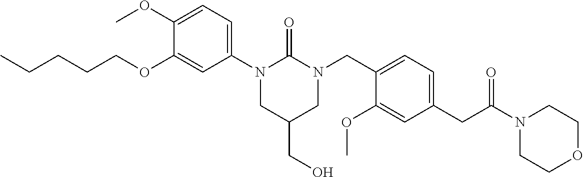 [SMILES: CCCCCOc1cc(N2CC(CO)CN(Cc3ccc(CC(=O)N4CCOCC4)cc3OC)C2=O)ccc1OC] |
| SC0184 | 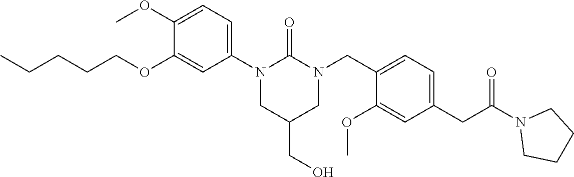 [SMILES: CCCCCOc1cc(N2CC(CO)CN(Cc3ccc(CC(=O)N4CCCC4)cc3OC)C2=O)ccc1OC] |
| SC0185 | 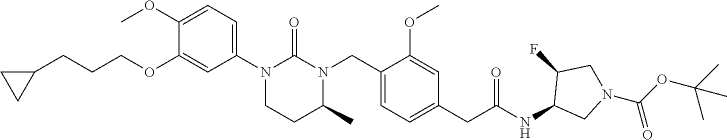 [SMILES: COc1cc(CC(=O)N[C@@H]2CN(C(=O)OC(C)(C)C)C[C@@H]2F)ccc1CN1C(=O)N(c2ccc(OC)c(OCCCC3CC3)c2)CC[C@@H]1C] |
| SC0186 | 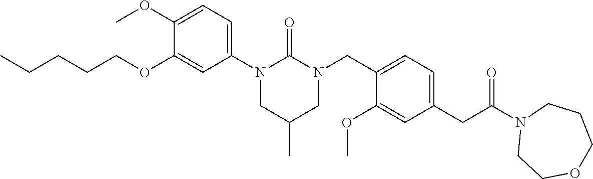 [SMILES: CCCCCOc1cc(N2CC(C)CN(Cc3ccc(CC(=O)N4CCCOCC4)cc3OC)C2=O)ccc1OC] |
| SC0187 | 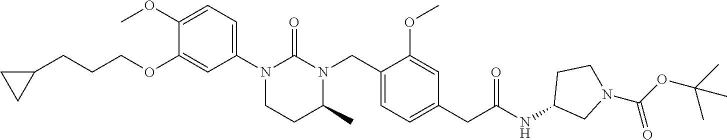 [SMILES: COc1cc(CC(=O)N[C@@H]2CCN(C(=O)OC(C)(C)C)C2)ccc1CN1C(=O)N(c2ccc(OC)c(OCCCC3CC3)c2)CC[C@@H]1C] |
| SC0188 | 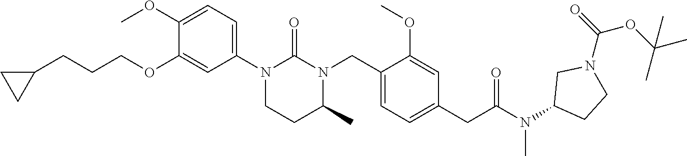 [SMILES: COc1cc(CC(=O)N(C)[C@H]2CCN(C(=O)OC(C)(C)C)C2)ccc1CN1C(=O)N(c2ccc(OC)c(OCCCC3CC3)c2)CC[C@@H]1C] |
| SC0189 | 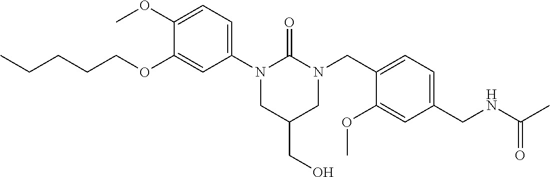 [SMILES: CCCCCOc1cc(N2CC(CO)CN(Cc3ccc(CNC(C)=O)cc3OC)C2=O)ccc1OC] |
| SC0190 | 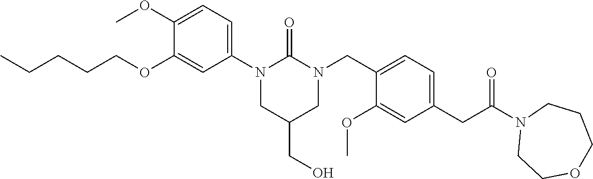 [SMILES: CCCCCOc1cc(N2CC(CO)CN(Cc3ccc(CC(=O)N4CCCOCC4)cc3OC)C2=O)ccc1OC] |
| SC0191 | 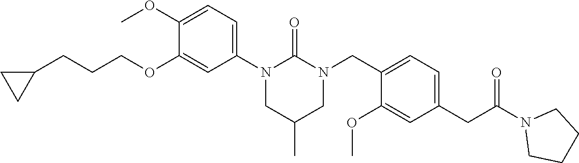 [SMILES: COc1cc(CC(=O)N2CCCC2)ccc1CN1CC(C)CN(c2ccc(OC)c(OCCCC3CC3)c2)C1=O] |
| SC0192 | 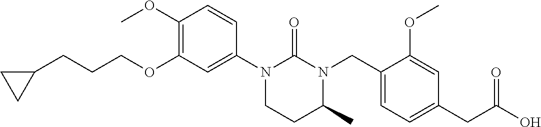 [SMILES: COc1cc(CC(=O)O)ccc1CN1C(=O)N(c2ccc(OC)c(OCCCC3CC3)c2)CC[C@@H]1C] |
| SC0193 | 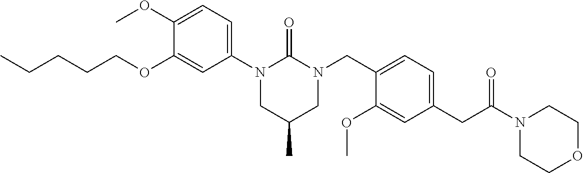 [SMILES: CCCCCOc1cc(N2C[C@H](C)CN(Cc3ccc(CC(=O)N4CCOCC4)cc3OC)C2=O)ccc1OC] |
| SC0194 | 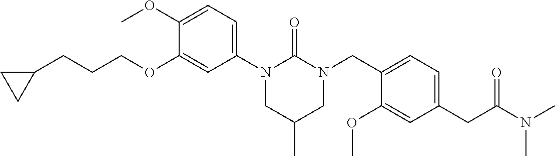 [SMILES: COc1cc(CC(=O)N(C)C)ccc1CN1CC(C)CN(c2ccc(OC)c(OCCCC3CC3)c2)C1=O] |
| SC0195 | 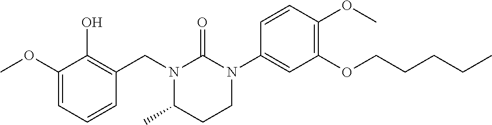 [SMILES: CCCCCOc1cc(N2CC[C@H](C)N(Cc3cccc(OC)c3O)C2=O)ccc1OC] |
| SC0196 | 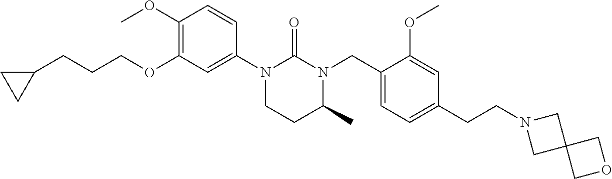 [SMILES: COc1cc(CCN2CC3(COC3)C2)ccc1CN1C(=O)N(c2ccc(OC)c(OCCCC3CC3)c2)CC[C@@H]1C] |
| SC0197 | 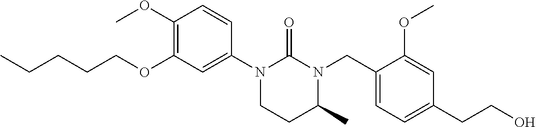 [SMILES: CCCCCOc1cc(N2CC[C@H](C)N(Cc3ccc(CCO)cc3OC)C2=O)ccc1OC] |
| SC0198 | 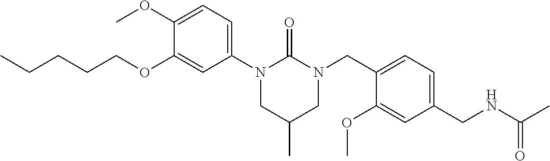 [SMILES: CCCCCOc1cc(N2CC(C)CN(Cc3ccc(CNC(C)=O)cc3OC)C2=O)ccc1OC] |
| SC0199 | 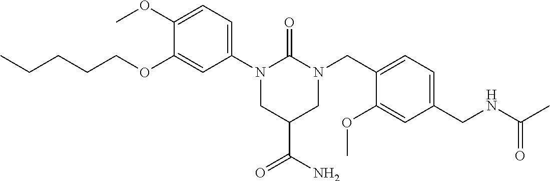 [SMILES: CCCCCOc1cc(N2CC(C(N)=O)CN(Cc3ccc(CNC(C)=O)cc3OC)C2=O)ccc1OC] |
| SC0200 | 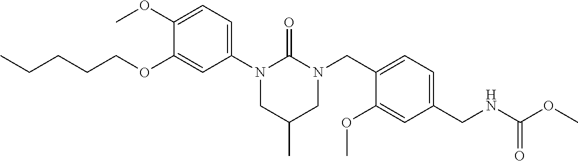 [SMILES: CCCCCOc1cc(N2CC(C)CN(Cc3ccc(CNC(=O)OC)cc3OC)C2=O)ccc1OC] |
| SC0201 | 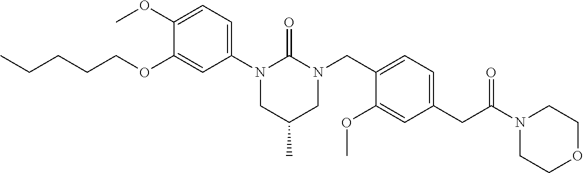 [SMILES: CCCCCOc1cc(N2C[C@@H](C)CN(Cc3ccc(CC(=O)N4CCOCC4)cc3OC)C2=O)ccc1OC] |
| SC0202 | 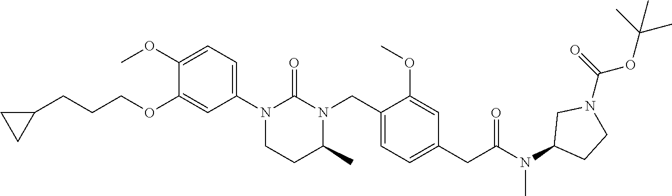 [SMILES: COc1cc(CC(=O)N(C)[C@@H]2CCN(C(=O)OC(C)(C)C)C2)ccc1CN1C(=O)N(c2ccc(OC)c(OCCCC3CC3)c2)CC[C@@H]1C] |
| SC0203 | 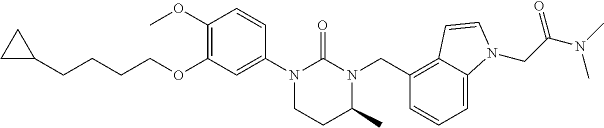 [SMILES: COc1ccc(N2CC[C@H](C)N(Cc3cccc4c3ccn4CC(=O)N(C)C)C2=O)cc1OCCCCC1CC1] |
| SC0204 | 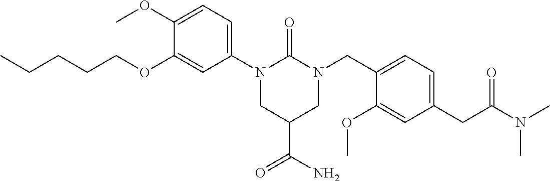 [SMILES: CCCCCOc1cc(N2CC(C(N)=O)CN(Cc3ccc(CC(=O)N(C)C)cc3OC)C2=O)ccc1OC] |
| SC0205 | 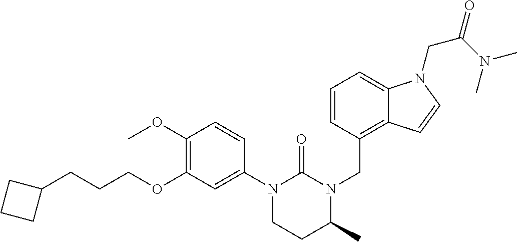 [SMILES: COc1ccc(N2CC[C@H](C)N(Cc3cccc4c3ccn4CC(=O)N(C)C)C2=O)cc1OCCCC1CCC1] |
| SC0206 | 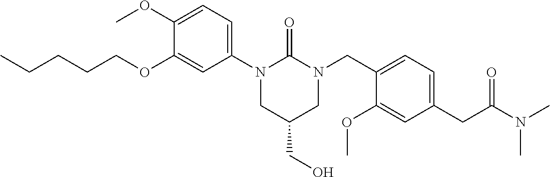 [SMILES: CCCCCOc1cc(N2C[C@@H](CO)CN(Cc3ccc(CC(=O)N(C)C)cc3OC)C2=O)ccc1OC] |
| SC0207 | 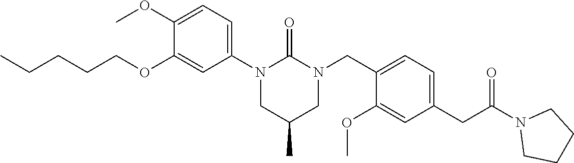 [SMILES: CCCCCOc1cc(N2C[C@H](C)CN(Cc3ccc(CC(=O)N4CCCC4)cc3OC)C2=O)ccc1OC] |
| SC0208 | 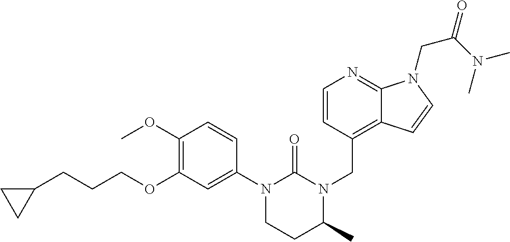 [SMILES: COc1ccc(N2CC[C@H](C)N(Cc3ccnc4c3ccn4CC(=O)N(C)C)C2=O)cc1OCCCC1CC1] |
| SC0209 | 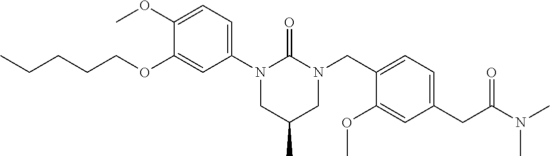 [SMILES: CCCCCOc1cc(N2C[C@H](C)CN(Cc3ccc(CC(=O)N(C)C)cc3OC)C2=O)ccc1OC] |
| SC0210 | 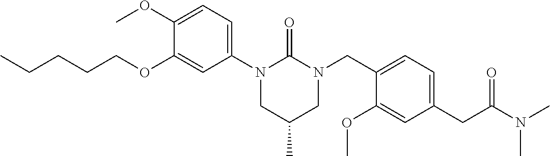 [SMILES: CCCCCOc1cc(N2C[C@@H](C)CN(Cc3ccc(CC(=O)N(C)C)cc3OC)C2=O)ccc1OC] |
| SC0211 | 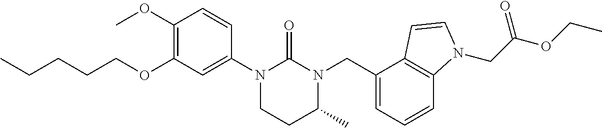 [SMILES: CCCCCOc1cc(N2CC[C@@H](C)N(Cc3cccc4c3ccn4CC(=O)OCC)C2=O)ccc1OC] |
| SC0212 | 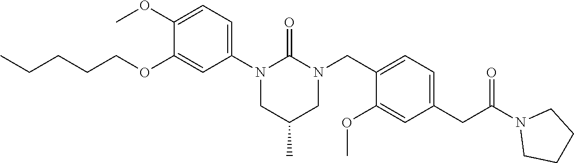 [SMILES: CCCCCOc1cc(N2C[C@@H](C)CN(Cc3ccc(CC(=O)N4CCCC4)cc3OC)C2=O)ccc1OC] |
| SC0213 | 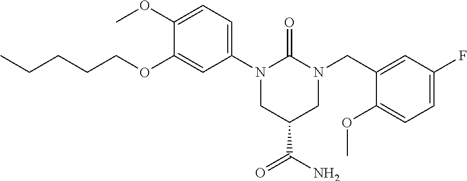 [SMILES: CCCCCOc1cc(N2C[C@@H](C(N)=O)CN(Cc3cc(F)ccc3OC)C2=O)ccc1OC] |
| SC0214 | 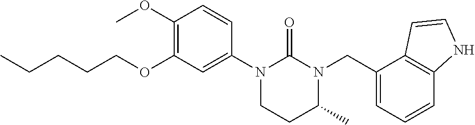 [SMILES: CCCCCOc1cc(N2CC[C@@H](C)N(Cc3cccc4[nH]ccc34)C2=O)ccc1OC] |
| SC0215 | 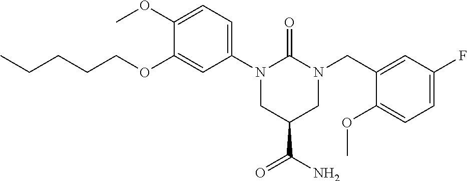 [SMILES: CCCCCOc1cc(N2C[C@H](C(N)=O)CN(Cc3cc(F)ccc3OC)C2=O)ccc1OC] |
| SC0216 | 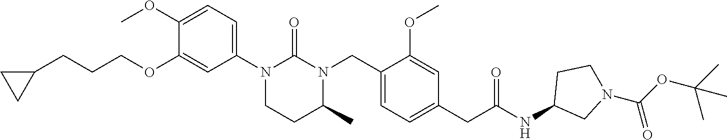 [SMILES: COc1cc(CC(=O)N[C@H]2CCN(C(=O)OC(C)(C)C)C2)ccc1CN1C(=O)N(c2ccc(OC)c(OCCCC3CC3)c2)CC[C@@H]1C] |
| SC0217 | 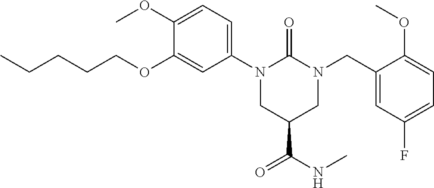 [SMILES: CCCCCOc1cc(N2C[C@H](C(=O)NC)CN(Cc3cc(F)ccc3OC)C2=O)ccc1OC] |
| SC0218 | 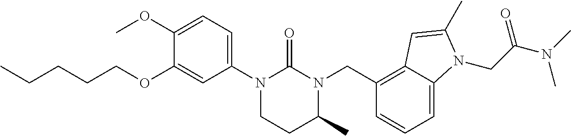 [SMILES: CCCCCOc1cc(N2CC[C@H](C)N(Cc3cccc4c3cc(C)n4CC(=O)N(C)C)C2=O)ccc1OC] |
| SC0219 | 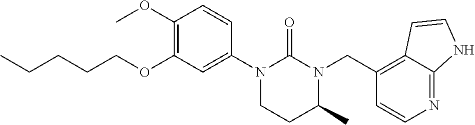 [SMILES: CCCCCOc1cc(N2CC[C@H](C)N(Cc3ccnc4[nH]ccc34)C2=O)ccc1OC] |
| SC0220 | 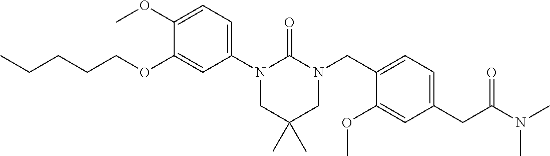 [SMILES: CCCCCOc1cc(N2CC(C)(C)CN(Cc3ccc(CC(=O)N(C)C)cc3OC)C2=O)ccc1OC] |
| SC0221 | 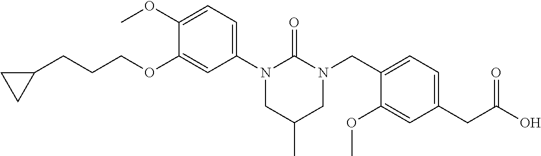 [SMILES: COc1cc(CC(=O)O)ccc1CN1CC(C)CN(c2ccc(OC)c(OCCCC3CC3)c2)C1=O] |
| SC0222 | 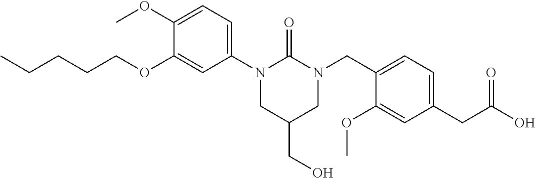 [SMILES: CCCCCOc1cc(N2CC(CO)CN(Cc3ccc(CC(=O)O)cc3OC)C2=O)ccc1OC] |
| SC0223 | 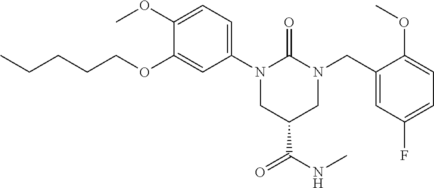 [SMILES: CCCCCOc1cc(N2C[C@@H](C(=O)NC)CN(Cc3cc(F)ccc3OC)C2=O)ccc1OC] |
| SC0224 | 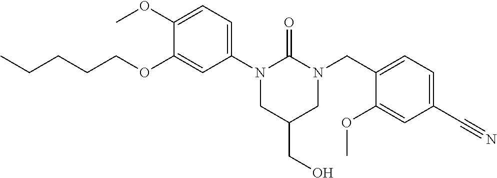 [SMILES: CCCCCOc1cc(N2CC(CO)CN(Cc3ccc(C#N)cc3OC)C2=O)ccc1OC] |
| SC0225 | 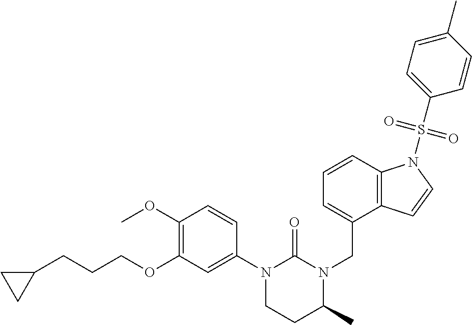 [SMILES: COc1ccc(N2CC[C@H](C)N(Cc3cccc4c3ccn4S(=O)(=O)c3ccc(C)cc3)C2=O)cc1OCCCC1CC1] |
| SC0226 | 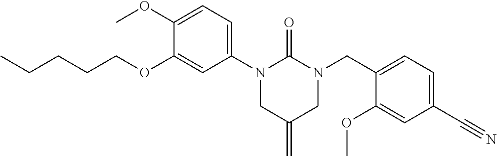 [SMILES: C=C1CN(Cc2ccc(C#N)cc2OC)C(=O)N(c2ccc(OC)c(OCCCCC)c2)C1] |
| SC0227 | 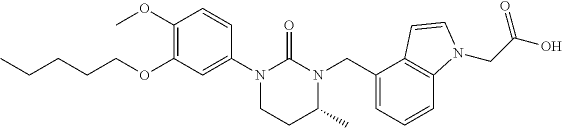 [SMILES: CCCCCOc1cc(N2CC[C@@H](C)N(Cc3cccc4c3ccn4CC(=O)O)C2=O)ccc1OC] |
| SC0228 | 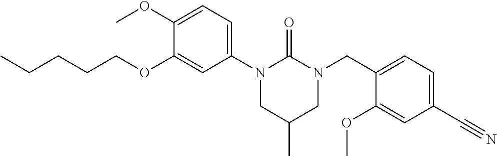 [SMILES: CCCCCOc1cc(N2CC(C)CN(Cc3ccc(C#N)cc3OC)C2=O)ccc1OC] |
| SC0229 | 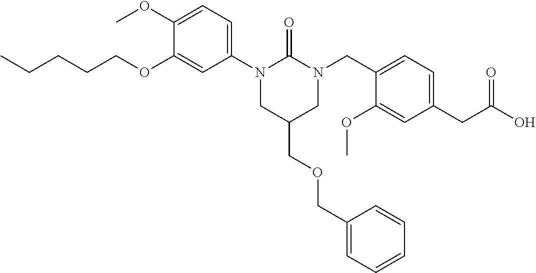 [SMILES: CCCCCOc1cc(N2CC(COCc3ccccc3)CN(Cc3ccc(CC(=O)O)cc3OC)C2=O)ccc1OC] |
| SC0230 | 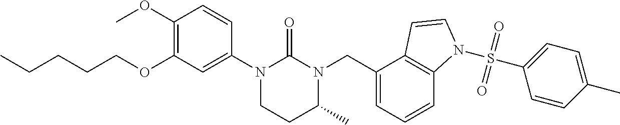 [SMILES: CCCCCOc1cc(N2CC[C@@H](C)N(Cc3cccc4c3ccn4S(=O)(=O)c3ccc(C)cc3)C2=O)ccc1OC] |
| SC0231 | 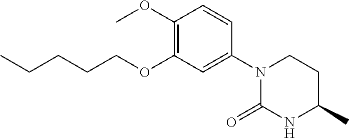 [SMILES: CCCCCOc1cc(N2CC[C@@H](C)NC2=O)ccc1OC] |
| SC0232 | 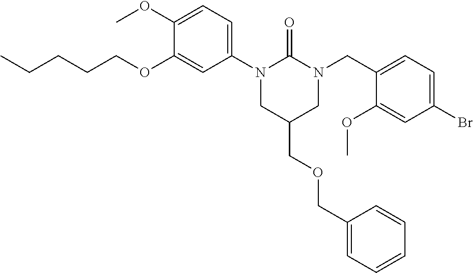 [SMILES: CCCCCOc1cc(N2CC(COCc3ccccc3)CN(Cc3ccc(Br)cc3OC)C2=O)ccc1OC] |

### Generic Structure—Formula (IIIa)

In some embodiments, a C5 inhibitor compound of the present disclosure has a structure according to Formula (IIa):

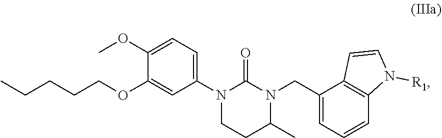

or a pharmaceutically acceptable salt thereof,
wherein R₁ is —CH₂—CO—R₂ or —CH₂—R₂ wherein R₂ is an amine group, an alkyl group, an aryl group, pyridine, indole, or

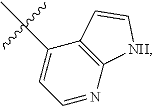 [SMILES: *c1ccnc2[nH]ccc12]

wherein each of them is optionally further substituted.

Non-limiting examples of C5 inhibitor compounds having a structure according to Formula (IIIa) include SC0001, SC0002, SC0003, SC0004, SC0005, SC0006, SC0007, SC0008, SC0009, SC0010, SC0011, SC0012, SC0013, SC0014, and SC0015.

### Generic Structure—Formula (IIb)

In some embodiments, a C5 inhibitor compound of the present disclosure has a structure according to Formula (IIIb):

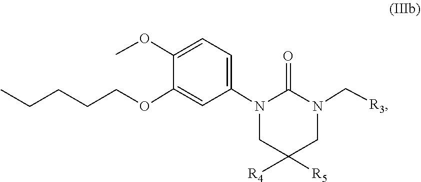

or a pharmaceutically acceptable salt thereof. R₃ may be any group that has an amide (—CO—NH—) or a phenyl group, wherein each group is optionally further substituted, such as with at least one alkyl group, alkoxyl group, or halogen. R₄ may be —H or —OH. R₅ may be —CH₃, —CH₂OH, or —CH₂NH₂, wherein each group is optionally further substituted.

In some embodiments, R₈ comprises a nitrogen atom and the nitrogen atom may be part of a cyclic or bicyclic structure (saturated or non-saturated).

Non-limiting examples of C5 inhibitor compounds having a structure according to Formula (IIIb) include SC0016, SC0017, SC0018, SC0019, SC0020, SC0021, SC0022, SC0023, SC0024, SC0025, SC0026, SC0027, SC0028, SC0029, SC0030, SC0031, SC0032, SC0033, SC0034, SC0035, SC0036, SC0037, SC0038, SC0039, SC0040, SC0041, SC0042, SC0043, and SC0072.

### Generic Structure—Formula (IIb1)

In some embodiments, a C5 inhibitor compound of the present disclosure has a structure according to Formula (IIIb1):

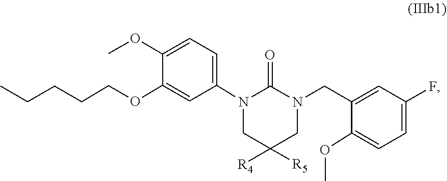

or a pharmaceutically acceptable salt thereof. R4 may be —H or —OH. R₈ may be —CH₃, —CH₂OH or —CH₂NH₂, wherein each group is optionally further substituted. In some embodiments, the nitrogen atom may be part of a cyclic or bicyclic structure (saturated or non-saturated).

Non-limiting examples of C5 inhibitor compounds having a structure according to Formula (IIIb1) include SC0016, SC0018, SC0020, SC0021, SC0022, SC0023, SC0024, SC0025, SC0026, SC0027, SC0028, SC0029, SC0030, SC0031, SC0032, SC0033, SC0034, SC0035, SC0036, SC0037, SC0038, SC0039, SC0040, SC0041, SC0042, and SC0043.

### Generic Structure—Formula (IIIc)

In some embodiments, a C5 inhibitor compound of the present disclosure has a structure according to Formula (IIIc):

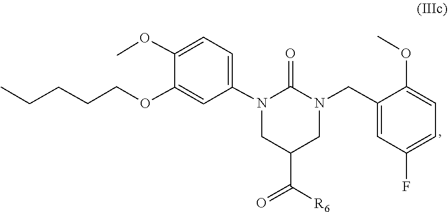

or a pharmaceutically acceptable salt thereof. R₆ may be an amine group, optionally further substituted with any suitable functional group, such as an alkyl, an alkoxyl, a cyclic group, a heterocyclic group, an aryl group, or a heteroaryl group. The nitrogen in the amine group may be part of a heterocyclic or heteroaryl group. The hetero atom may be nitrogen, sulfur, or oxygen.

In some embodiments, R6 is

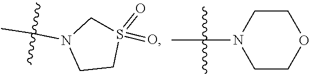 [SMILES: *N1CCS(=O)(=O)C1 ; *N1CCOCC1]

or any substituted derivative thereof.

Non-limiting examples of C5 inhibitor compounds having a structure according to Formula (IIIc) include SC0044, SC0045, SC0046, SC0047, SC0048, SC0049, SC0050, and SC0051.

### Generic Structure—Formula (IIId)

In some embodiments, a C5 inhibitor compound of the present disclosure has a structure according to Formula (IIId):

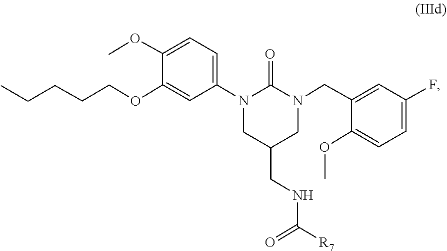

or a pharmaceutically acceptable salt thereof. R₇ may be an alkyl group, an amide group, a cyclic group, a heterocyclic group, an aryl group, or a heteroaryl group. The hetero atom may be nitrogen, oxygen, or sulfur. Each group may optionally be further substituted with any suitable functional group, such as at least one alkyl, alkoxyl, or halogen.

In some embodiments, R7 is oxazole, pyridine, pyrazole, or any substituted derivative thereof.

Non-limiting examples of C5 inhibitor compounds having a structure according to Formula (IIId) include SC0052, SC0036, SC0053, SC0054, SC0055, SC0056, and SC0057.

### Generic Structure—Formula (IIIe)

In some embodiments, a C5 inhibitor compound of the present disclosure has a structure according to Formula (IIIe):

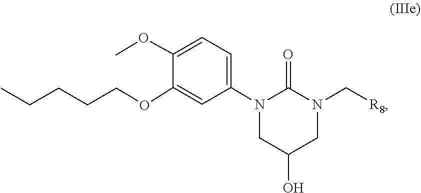

or pharmaceutically acceptable salt thereof. R8 may be a phenyl group and may optionally be further substituted, such as with at least one alkyl group, alkoxyl group, or halogen.

Non-limiting examples of C5 inhibitor compounds having a structure according to Formula (IIIe) include SC0058.

### Generic Structure—Formula (IIIf)

In some embodiments, a C5 inhibitor compound of the present disclosure has a structure according to Formula (IIIf):

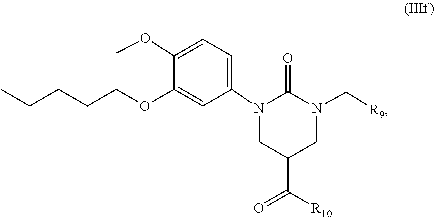

or a pharmaceutically acceptable salt thereof. R₉ may be a phenyl group and may optionally be further substituted, such as with at least one alkyl group, alkoxyl group, or halogen. R₁₀ may be an alkyl group, an alkoxyl group, or —OH.

Non-limiting examples of C5 inhibitor compounds having a structure according to Formula (IIIf) include SC0059.

### Generic Structure—Formula (IIIg)

In some embodiments, a C5 inhibitor compound of the present disclosure has a structure according to Formula (IIIg):

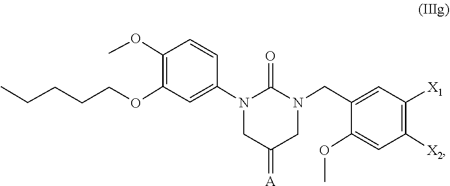

or a pharmaceutically acceptable salt thereof. A may be carbon or oxygen. X₁ and X2 may be independently hydrogen, a halogen, an alkyl, or an alkoxyl group.

Non-limiting examples of C5 inhibitor compounds having a structure according to Formula (IIIg) include SC0060, SC0061, and SC0062.

### Generic Structure—Formula (IIIg1)

In some embodiments, a C5 inhibitor compound of the present disclosure has a structure according to Formula (IIIg1):

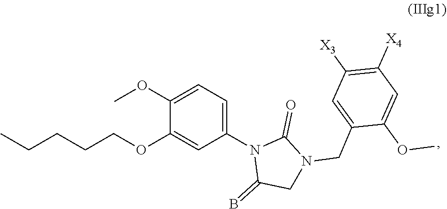

or a pharmaceutically acceptable salt thereof. B may be carbon or oxygen. X₃ and X₄ may be independently hydrogen, a halogen, an alkyl, or an alkoxyl group.

Non-limiting examples of C5 inhibitor compounds having a structure according to Formula (VIIa) include SC0063.

### Generic Structure—Formula (IIIh)

In some embodiments, a C5 inhibitor compound of the present disclosure has a structure according to Formula (IIIh):

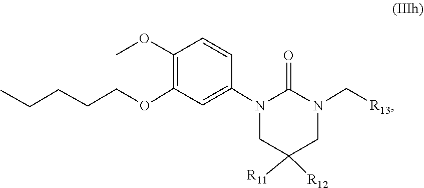

or a pharmaceutically acceptable salt thereof. R₁₁ and R12 may together form a cyclic group, a heterocyclic group, an aryl group, or a heteroaryl group. The hetero atom may be nitrogen, oxygen, or sulfur. Each group may optionally be further substituted with any suitable functional group. Such groups may include at least one —COO—, —SO₂—, and/or halogen. R₁₃ may include

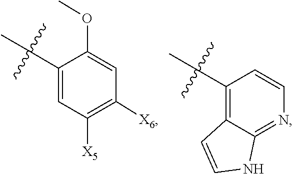

or a substituted derivative thereof. X₅ and X₆ may be independently hydrogen, a halogen, an alkyl, or an alkoxyl group. In some embodiments, R₁₃ is

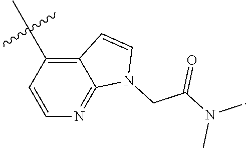 [SMILES: *c1ccnc2c1ccn2CC(=O)N(C)C]

Non-limiting examples of C5 inhibitor compounds having a structure according to Formula (IIIh) include SC0064, SC0065, SC0066, SC0067, SC0068, SC0069, SC0070, and SC0071.

### Generic Structure—Formula (IIIi)

In some embodiments, a C5 inhibitor compound of the present disclosure has a structure according to Formula (IIIi):

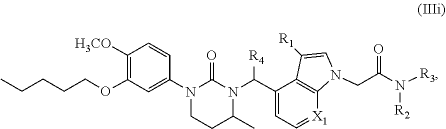

or a pharmaceutically acceptable salt thereof, PGP
wherein X1 is CH or N;
wherein R1 is H, halogen (such as Cl, F, Br or I), —CN, —CF3, or a C1-C3 alkyl group;
wherein R2 or R3, independently, is H, alkyl, aryl, heteroaryl, cyclic alkyl, heterocyclic alkyl, a multicyclic alkyl group, or a hetero multicyclic alkyl group, wherein the alkyl, aryl, heteroaryl group, cyclic alkyl, heterocyclic alkyl, multicyclic alkyl, or hetero multicyclic alkyl group is optional substituted; optionally, R2 and R3, together with the nitrogen they are attached, form a 3 to 8 membered heterocyclic group, wherein the heterocyclic group may be optionally substituted; and
wherein R4 is H or a C1-C3 alkyl group.

In some embodiments, R1 is H.

In some embodiments, R2 and R3, together with the nitrogen they are attached to, form a 6-membered non-aromatic heterocyclic group. In some embodiments, the heterocyclic group is

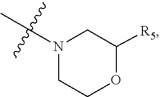

wherein R5 is an alkyl group, wherein the alkyl group is optionally substituted. In some embodiments, R5 is an alkyl group substituted with an amine group.

Non-limiting examples of C5 inhibitor compounds having a structure according to Formula (IIIi) include SC0001, SC0002, SC0003, SC0004, SC0005, SC0006, SC0007, SC0008, SC0009, SC0011, SC0012, SC0014, SC0100, SC0103, SC0105, SC0106, SC0107, SC0108, SC0109, SC0110, SC0111, SC0112, SC0113, SC0117, SC0120, SC0122, SC0124, SC0127, SC0128, SC0129, SC0133, SC0143, SC0147, SC0154, SC0155, SC0156, SC0171, and SC0177.

C5 inhibitor compounds of the present disclosure may be directed to chemically stable and feasible compounds. A chemical compound is considered to be feasible and stable when the chemical structure of the compound is not significantly altered when stored at a temperature of 40° C. or less in the absence of chemically reactive conditions, such as moisture for a period of one week.

Unless otherwise stated, structures presented herein can include all stereochemical forms of the structure; i.e., the R and S configurations for each asymmetric center. All stereoisomers, such as enantiomers, diastereomers and geometric isomers are intended unless otherwise indicated. Therefore, single stereochemical isomers as well as enantiomeric, diastereomeric and cis/trans mixtures of the present compounds are within the scope of the present disclosure. Cis and trans geometric isomers of the compounds of the present disclosure are described and may be isolated as a mixture of isomers or as separated isomeric forms.

Unless otherwise stated, structures presented herein are also meant to include compounds which differ only in the presence of one or more isotopically enriched atoms. For example, replacements of a hydrogen atom by deuterium or tritium, or carbon atom by a ¹³C- or ¹⁴C-enriched carbon are within the scope of the present disclosure.

Unless otherwise indicated, compounds of the present disclosure can exist in alternative tautomeric forms.

In some embodiments, C5-interacting compounds may be selected based on kinetic and/or thermodynamic solubility. Compound solubility may be an important feature for ease of manufacturing and/or use of compounds in formulations or other therapeutic formats. Thermodynamic solubility refers to the ability of a compound to dissolve in a certain volume of a specific solvent at a given temperature. Kinetic solubility refers to solubility in an aqueous solvent when a compound is added from a high concentration organic solvent stock solution. A kinetic solubility value can be obtained by determining the maximum concentration of dissolved compound that can be achieved in an aqueous solvent when prepared from a high concentration organic solvent (e.g., DMSO). This value may be determined using HPLC-UV or LC-MS/MS analysis of the final solution after filtering out any undissolved compound. In some embodiments, C5-interacting compounds of the present disclosure exhibit a kinetic solubility value of from about 10 μM to about 500 μM, wherein the organic solvent is DMSO and the aqueous solvent is 0.5 M phosphate buffered saline, pH 7.4. The kinetic solubility value may be from about 20 μM to about 50 μM.

In some embodiments, C5-interacting compounds may be selected based on cell permeability. Cell permeability may be assessed using cell-based permeability assays. Such assays may include the use of cultured cell monolayers on a semi-permeable membrane wherein compounds are introduced to a chamber above or below the cell monolayer and concentration of the compound in a chamber on the opposite side of the cell monolayer is determined over time. Such analysis may be used to calculate an apparent permeability (Pₐₚₚ) value that represents the rate of movement of compound across the cell monolayer. In some embodiments, Madin Darby canine kidney (MDCK) cell monolayers may be used. Unidirectional transport may be assessed using MDCK wild type (MDCK-WT) cell monolayers. For bidirectional transport assessment, MDCK-MDR1 cell monolayers may be used. MDCK-MDR1 cells express the MDR1 gene encoding the P-glycoprotein (P-gp) efflux protein. This system may be used to assess bidirectional transport by calculating an efflux ratio for a given compound analyzed. The efflux ratio is determined by obtaining a Pₐₚₚ value for apical to basolateral compound movement (Pₐₚₚ A-B) across the MDCK cell monolayer; obtaining a Pₐₚₚ value for basolateral to apical movement (Pap B-A) across the MDCK cell monolayer; and calculating the ratio of Pₐₚₚ A-B to Pₐₚₚ B-A. In some embodiments, C5-interacting compounds of the present disclosure exhibit a Pₐₚₚ value for movement across MDCK cell monolayers of from about 0.1×10⁻⁶ cm/s to about 30×10⁻⁶ cm/s, wherein the Pₐₚₚ value is determined by measuring apical to basolateral movement across MDCK cell monolayer. In some embodiments, C5-interacting compounds of the present disclosure may exhibit an efflux ratio of from about 5 to about 150, wherein the efflux ratio is determined by obtaining a Pₐₚₚ value for apical to basolateral movement (Pₐₚₚ A-B) across a MDCK-MDR1 cell monolayer; obtaining a Pap value for basolateral to apical movement (Pₐₚₚ B-A) across the MDCK-MDR1 cell monolayer; and calculating the ratio of Pₐₚₚ A-B to Pₐₚₚ B-A.

### Compound Synthesis

Compounds of the present disclosure may be synthesized according to standard methods known in the art [see, e.g. Morrison and Boyd in “Organic Chemistry”, 6^{th} edition, Prentice Hall (1992), the contents of which are herein incorporated by reference in their entirety]. Some compounds and/or intermediates of the present disclosure may be commercially available, known in the literature, or readily obtainable by those skilled in the art using standard procedures. Some compounds of the present disclosure may be synthesized using schemes, examples, or intermediates described herein. Where the synthesis of a compound, intermediate or variant thereof is not fully described, those skilled in the art can recognize that the reaction time, number of equivalents of reagents and/or temperature may be modified from reactions described herein to prepare compounds presented or intermediates or variants thereof and that different work-up and/or purification techniques may be necessary or desirable to prepare such compounds, intermediates, or variants.

Synthetic reactions may be carried out under various temperatures and/or atmospheric conditions to achieve desired results. Temperatures used in compound synthesis may be varied between −273.16° C. and 150° C., or greater than 150° C. In some embodiments, synthetic reactions are carried out from about −75° C. to about −40° C., from about −40° C. to about 25° C., from about 0° C. to about 50° C., from about 40° C. to about 80° C., from about 50° C. to about 85° C., from about 65° C. to about 90° C., from about 70° C. to about 95° C., from about 75° C. to about 100° C., from about 80° C. to about 110° C., from about 85° C. to about 120° C., from about 90° C. to about 140° C., or from about 100° C. to about 150° C.

Atmospheric conditions may be varied to include various levels of gases. Such gases may include, but are not limited to, oxygen, nitrogen, hydrogen, carbon dioxide, and carbon monoxide. Atmospheric conditions may also be varied by pressure to achieve a desired reaction. Atmospheric pressures may be varied, for example, by from about 0 psi to about 1000 psi (e.g., from about 0 psi to about 20 psi, from about 10 psi to about 50 psi, from about 40 psi to about 200 psi, from about 75 psi to about 500 psi, or from about 150 psi to about 1000 psi). In some embodiments, reactions are carried out under microwave irradiation.

Synthetic reactions may be carried out in various reaction mixtures. Reaction mixtures may include water or other solvents. Such solvents may include organic or hydrophobic solvents. Reaction mixtures may be formulated with various compounds to alter one or more of pH and salinity. In some embodiments, reaction mixtures may include one or more reaction compounds. Reaction compounds may include reactants, catalysts, and/or other chemicals necessary for facilitating chemical reactions.

Filtration, concentration, and/or purification of compounds presented herein (or intermediates or variants thereof) may be carried out according to methods known in the art. Examples of purification methods may include chromatography, e.g., column chromatography. Chromatography may include, but is not limited to, one or more of thin-layer chromatography (TLC), preparative TLC (prep-TLC), normal phase chromatography, silica gel chromatography, flash silica gel chromatography, high performance liquid chromatography (HPLC), preparative HPLC (prep-HPLC), reverse phase column chromatography, reverse phase flash chromatography, C18 reverse phase flash chromatography, and C18 reverse phase HPLC. Filtration may be carried out, in some embodiments, over celite. Removal of water may be carried out, in some embodiments, using a Dean-Stack apparatus. In some embodiments, solids may be extracted from solution by lyophilization. In some embodiments, preparations may be sonicated before subsequent reactions and/or purification. In some embodiments, filtration and/or concentration may be carried out under varying pressure to achieve desired results. In some cases, filtration, concentration, and/or purification may be carried out in a vacuum. Compound preparations resulting from filtration, concentration, and/or purification may be in liquid or solid form. Liquids preparations may include water or other solvents. Such solvents may include organic solvents or hydrophobic solvents. Some compound preparations may be in the form of an oil. Solid compound preparations may include different formats that include, but are not limited to blocks, crystalline or granular formats, or powders. Filtration, concentration, and/or purification may be carried out using an eluant. Eluants may include water or other solvents. Such solvents may include organic or hydrophobic solvents. Some eluants may include ethyl acetate, petroleum ether, hexane, or n-hexane.

Synthesized compounds may be validated for proper structure by methods known to those skilled in the art, for example by nuclear magnetic resonance (NMR) spectroscopy and/or mass spectrometry.

### Formulations

In some embodiments, compounds of the present disclosure may be included in a composition that includes one or more compounds and at least one excipient (e.g., a pharmaceutically acceptable excipient). Such compositions may include C5 inhibitors. Compounds may be present in compositions at various concentrations, including, but not limited to from about 0.001 mg/mL to about 0.2 mg/mL, from about 0.01 mg/mL to about 2 mg/mL, from about 0.1 mg/mL to about 10 mg/mL, from about 0.5 mg/mL to about 5 mg/mL, from about 1 mg/mL to about 20 mg/mL, from about 15 mg/mL to about 40 mg/mL, from about 25 mg/mL to about 75 mg/mL, from about 50 mg/mL to about 200 mg/mL, or from about 100 mg/mL to about 400 mg/mL.

In some embodiments, compositions of the present disclosure include aqueous compositions which include at least water and a C5 inhibitor compound. Aqueous C5 inhibitor compositions of the present disclosure may further include one or more salt and/or one or more buffering agent. In some cases, aqueous compositions of the present disclosure include water, a C5 inhibitor compound, a salt, and a buffering agent.

Aqueous C5 inhibitor formulations of the present disclosure may have pH levels of from about 2.0 to about 3.0, from about 2.5 to about 3.5, from about 3.0 to about 4.0, from about 3.5 to about 4.5, from about 4.0 to about 5.0, from about 4.5 to about 5.5, from about 5.0 to about 6.0, from about 5.5 to about 6.5, from about 6.0 to about 7.0, from about 6.5 to about 7.5, from about 7.0 to about 8.0, from about 7.5 to about 8.5, from about 8.0 to about 9.0, from about 8.5 to about 9.5, or from about 9.0 to about 10.0.

In some cases, compounds and compositions of the present disclosure are prepared according to good manufacturing practice (GMP) and/or current GMP (cGMP). Guidelines used for implementing GMP and/or cGMP can be obtained from one or more of the US Food and Drug Administration (FDA), the World Health Organization (WHO), and the International Conference on Harmonization (ICH).

### Pharmaceutical Compositions

In some embodiments, compounds of the present disclosure may be formulated as pharmaceutical compositions. The term “pharmaceutical composition” refers to a composition comprising at least one active ingredient (e.g., one or more compounds described herein) in a form and amount that permits the active ingredient to be therapeutically effective. In some embodiments, compounds may be formulated according to any of the techniques for preparing pharmaceutical formulations described in Remington: The Science and Practice of Pharmacy, 21st Edition, Lippincott Williams & Wilkins, (2005); and Encyclopedia of Pharmaceutical Technology, eds. J. Swarbrick and J. C. Boylan, 1988-1999, Marcel Dekker, New York, each of which is incorporated herein by reference. In some embodiments, C5 inhibitor compounds may be combined with one or more pharmaceutically acceptable excipient to form a pharmaceutical composition. As used herein, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable excipient,” as used herein, refers any ingredient other than the inventive compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, dispensing, or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol. In some embodiments, pharmaceutical compositions comprise one or more active compound ingredients together with ethanol, corn oil-mono-di-triglycerides, hydrogenated castor oil, DL-tocopherol, propylene glycol, gelatin, glycerol, colorants, flavors and sweeteners.

### II. Methods

In some embodiments, methods of the present disclosure include methods of modulating complement activity using C5-interacting compounds described herein. Such methods may include methods of modulating complement activity in biological systems by contacting such systems with C5-interacting compounds. The C5-interacting compounds may be C5 inhibitors disclosed herein. Biological systems may include, but are not limited to, cells, tissues, organs, bodily fluids, organisms, non-mammalian subjects, and mammalian subjects (e.g., humans).

In some embodiments, the present disclosure provides methods of inhibiting complement activity in a subject. In some cases, the percentage of complement activity inhibited in a subject may be at least 10%, at least 20%, at least 30%, at least 40%, at least 50%, at least 60%, at least 700, at least 80%, at least, 85%, at least 90%, at least 95%, at least 96%, at least 97%, at least 98%, at least 99%, at least 99.5%, or at least 99.9%. In some cases, this level of inhibition and/or maximum inhibition of complement activity may be achieved by from about 1 hour after administration to about 3 hours after administration, from about 2 hours after administration to about 4 hours after administration, from about 3 hours after administration to about 10 hours after administration, from about 5 hours after administration to about 20 hours after administration, or from about 12 hours after administration to about 24 hours after administration. Inhibition of complement activity may continue throughout a period of at least 1 day, of at least 2 days, of at least 3 days, of at least 4 days, of at least 5 days, of at least 6 days, of at least 7 days, of at least 2 weeks, of at least 3 weeks, of at least 4 weeks, of at least 8 weeks, of at least 3 months, of at least 6 months, or at least 1 year. In some cases, this level of inhibition may be achieved through daily administration. Such daily administration may include administration for at least 2 days, for at least 3 days, for at least 4 days, for at least 5 days, for at least 6 days, for at least 7 days, for at least 2 weeks, for at least 3 weeks, for at least 4 weeks, for at least 2 months, for at least 4 months, for at least 6 months, for at least 1 year, or for at least 5 years. In some cases, subjects may be administered compounds or compositions of the present disclosure for the life of such subjects.

In some embodiments, compounds of the present disclosure may be used in assays used to assess complement activation and/or inhibition. Some assays may include diagnostic assays. In some cases, compounds may be included in methods of drug discovery. In some embodiments, methods of the present disclosure include use of C5-interacting compounds of the present disclosure to assess C5 binding by other compounds. Such methods may include conjugating C5-interacting compounds with one or more detectable labels (e.g., fluorescent dyes) and measuring C5 dissociation (via detectable label detection) in the presence of the other compounds. The detectable labels may include fluorescent compounds.

### Therapeutic Indications

In some embodiments, methods of the present disclosure include methods of treating therapeutic indications using compounds and/or compositions disclosed herein. As used herein, the term “therapeutic indication” refers to any symptom, condition, disorder, or disease that may be alleviated, stabilized, improved, cured, or otherwise addressed by some form of treatment or other therapeutic intervention (e.g., through complement inhibitor administration). Therapeutic indications may include, but are not limited to, inflammatory indications, wounds, injuries, autoimmune indications, vascular indications, neurological indications, kidney-related indications, ocular indications, cardiovascular indications, pulmonary indications, and pregnancy-related indications. Therapeutic indications associated with complement activity and/or dysfunction are referred to herein as “complement-related indications.” In some embodiments, methods of the present disclosure may include treating complement-related indications by administering compounds and/or compositions disclosed herein (e.g., complement inhibitor compounds).

In some embodiments, complement inhibitor compounds may be useful in the treatment of complement-related indications where complement activation leads to progression of a disease, disorder and/or condition. Such complement-related indications may include, but are not limited to inflammatory indications, wounds, injuries, autoimmune indications, vascular indications, neurological indications, kidney-related indications, ocular indications, cardiovascular indications, pulmonary indications, and pregnancy-related indications. Complement-related indications may include, but are not limited to, any of those listed in US Publication No. US2013/091285, the contents of which are herein incorporated by reference in their entirety.

Complement inhibitor compounds and compositions may be useful in the treatment of infectious diseases, disorders and/or conditions, for example, in a subject having an infection. In some embodiments, subjects having an infection or that are at risk of developing sepsis or a septic syndrome may be treated with complement inhibitors described herein. In some cases, complement inhibitor compounds may be used in the treatment of sepsis.

Complement inhibitor compounds and compositions may also be administered to improve the outcome of clinical procedures wherein complement inhibition is desired. Such procedures may include, but are not limited to grafting, transplantation, implantation, catheterization, intubation and the like. In some embodiments, complement inhibitor compounds and compositions are used to coat devices, materials and/or biomaterials used in such procedures. In some embodiments, the inner surface of a tube may be coated with compounds and compositions to prevent complement activation within a bodily fluid that passes through the tube, either in vivo or ex vivo, e.g., extracorporeal shunting, e.g., dialysis and cardiac bypass.

As used herein the terms “treat,” “treatment,” and the like, refer to relief from or alleviation of pathological processes. In the context of the present disclosure insofar as it relates to any of the other conditions recited herein below, the terms “treat,” “treatment,” and the like mean to relieve or alleviate at least one symptom associated with such condition, or to slow or reverse the progression or anticipated progression of such condition.

By “lower” or “reduce” in the context of a disease marker or symptom is meant a significant decrease in such a level, often statistically significant. The decrease may be, for example, at least 10%, at least 20%, at least 30%, at least 40% or more, and is preferably down to a level accepted as within the range of normal for an individual without such a disorder.

By “increase” or “raise” in the context of a disease marker or symptom is meant a significant rise in such level, often statistically significant. The increase may be, for example, at least 10%, at least 20%, at least 30%, at least 40% or more, and is preferably up to a level accepted as within the range of normal for an individual without such disorder.

A treatment or preventive effect is evident when there is a significant improvement, often statistically significant, in one or more parameters of disease status, or by a failure to worsen or to develop symptoms where they would otherwise be anticipated. As an example, a favorable change of at least 10% in a measurable parameter of disease, and preferably at least 20%, 30%, 40%, 50% or more may be indicative of effective treatment. Efficacy for a given compound or composition may also be judged using an experimental animal model for the given disease as known in the art. When using an experimental animal model, efficacy of treatment is evidenced when a statistically significant modulation in a marker or symptom is observed.

### Paroxysmal Nocturnal Hemoglobinuria (PNH)

Complement-related indications may include paroxysmal nocturnal hemoglobinuria (PNH). In some embodiments, complement inhibitor compounds and compositions may be used to treat, prevent or delay development of PNH. In some embodiments, the treatment may be involved with the prevention of hemolysis of PNH erythrocytes in a dose dependent manner.

An acquired mutation in the phosphatidylinositol glycan anchor biosynthesis, class A (PIG-A) gene that originates from a multipotent hematopoietic stem cell results in a rare disease known as paroxysmal nocturnal hemoglobinuria (PNH) (Pu, J. J. et al., Paroxysmal nocturnal hemoglobinuria from bench to bedside. Clin Transl Sci. 2011 June; 4(3):219-24). PNH is characterized by bone marrow disorder, hemolytic anemia and thrombosis. The PIG-A gene product is necessary for the production of a glycolipid anchor, glycosylphosphatidylinositol (GPI), utilized to tether proteins to the plasma membrane. Two complement-regulatory proteins, CD55 and CD59, become nonfunctional in the absence of GPI. This leads to complement-mediated destruction of these cells. Complement inhibitors are particularly useful in the treatment of PNH. In some embodiments, compounds and compositions may be used to treat, prevent or delay development of Paroxysmal nocturnal hemoglobinuria (PNH) or anemias associated with complement. Subjects with PNH are unable to synthesize functional versions of the complement regulatory proteins CD55 and CD59 on hematopoietic stem cells. This results in complement-mediated hemolysis and a variety of downstream complications. As used herein, the term “downstream” or “downstream complication” refers to any event occurring after and as a result of another event. In some cases, downstream events are events occurring after and as a result of C5 cleavage and/or complement activation.

PNH is characterized by low hemoglobin, increased levels of lactate dehydrogenase and bilirubin, and decreased level of haptoglobin. Symptoms of PNH include symptoms of anemia, such as tiredness, headaches, dyspnea, chest pain, dizziness, and feeling of lightheadedness.

Current treatments for PNH include the use of eculizumab (Alexion Pharmaceuticals, Cheshire, Conn.). In some cases, eculizumab may be ineffective due to mutation in C5, short half-life, immune reaction, or other reason. In some embodiments, methods of the present disclosure include methods of treating subjects with PNH, wherein such subjects have been treated previously with eculizumab. In some cases, eculizumab is ineffective in such subjects, making treatment with compounds of the present disclosure important for therapeutic relief. In some embodiments, compounds of the present disclosure may be used to treat subjects that are resistant to eculizumab treatment. Such subjects may include subjects with the R885H/C polymorphism, which confers resistance to eculizumab. In some cases, compounds of the present disclosure are administered simultaneously or in conjunction with eculizumab therapy. In such cases, subjects may experience one or more beneficial effects of such combined treatment, including, but not limited to more effective relief, faster relief and/or fewer side effects.

### Inflammatory Indications

Therapeutic indications that may be addressed with compounds and/or compositions of the present disclosure may include inflammatory indications. As used herein, the term “inflammatory indication” refers to therapeutic indications that involve immune system activation. Inflammatory indications may include complement-related indications. Inflammation may be upregulated during the proteolytic cascade of the complement system. Although inflammation may have beneficial effects, excess inflammation may lead to a variety of pathologies (Markiewski et al. 2007. Am J Pathol. 17: 715-27). In some embodiments, complement inhibitor compounds and compositions of the present disclosure may be used to treat, prevent, or delay development of inflammatory indications. Inflammatory indications may include, but are not limited to, Acute Disseminated Encephalomyelitis (ADEM), Acute necrotizing hemorrhagic leukoencephalitis, Addison's disease, Agammaglobulinemia, Alopecia areata, Amyloidosis, Ankylosing spondylitis, Acute antibody-mediated rejection following organ transplantation, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune pancreatitis, Autoimmune retinopathy, Autoimmune thrombocytopenic purpura (ATP), Autoimmune thyroid disease, Autoimmune urticaria, Axonal & neuronal neuropathies, Bacterial sepsis and septic shock, Balo disease, Behcet's disease, Bullous pemphigoid, Cardiomyopathy, Castleman disease, Celiac disease, Chagas disease, Chronic fatigue syndrome, Chronic inflammatory demyelinating polyneuropathy (CIDP), Chronic recurrent multifocal osteomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Diabetes Type I, Discoid lupus, Dressier's syndrome, Endometriosis, Eosinophilic esophagitis, Eosinophilic fasciitis, Erythema nodosum, Experimental allergic encephalomyelitis, Evans syndrome, Fibromyalgia, Fibrosing alveolitis, Giant cell arteritis (temporal arteritis), Glomerulonephritis, Goodpasture's syndrome, Granulomatosis with Polyangiitis (GPA) see Wegener's, Graves' disease, Guillain-Barre syndrome, Hashimoto's encephalitis, Hashimoto's thyroiditis, Hemolytic anemia (including atypical hemolytic uremic syndrome and plasma therapy-resistant atypical hemolytic-uremic syndrome), Henoch-Schonlein purpura, Herpes gestationis, Hypogammaglobulinemia, Idiopathic thrombocytopenic purpura (ITP), IgA nephropathy, IgG4-related sclerosing disease, Immunoregulatory lipoproteins, Inclusion body myositis, Insulin-dependent diabetes (type1), Interstitial cystitis, Juvenile arthritis, Juvenile diabetes, Kawasaki syndrome, Lambert-Eaton syndrome, Large vessel vasculopathy, Leukocytoclastic vasculitis, Lichen planus, Lichen sclerosus, Ligneous conjunctivitis, Linear IgA disease (LAD), Lupus (SLE), Lyme disease, Meniere's disease, Microscopic polyangiitis, Mixed connective tissue disease (MCTD), Mooren's ulcer, Mucha-Habermann disease, Multiple endocrine neoplasia syndromes, Multiple sclerosis, Multifocal motor neuropathy, Myositis, Myasthenia gravis, Narcolepsy, Neuromyelitis optica (Devic's), Neutropenia, Ocular cicatricial pemphigoid, Optic neuritis, Osteoarthritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polyendocrinopathies, Polymyalgia rheumatica, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic Pulmonary fibrosis, Pyoderma gangrenosum, Pure red cell aplasia, Raynauds phenomenon, Reactive arthritis, Reflex sympathetic dystrophy, Reiter's syndrome, Relapsing polychondritis, Restless legs syndrome, Retroperitoneal fibrosis, Rheumatic fever, Rheumatoid arthritis, Sarcoidosis, Schmidt syndrome, Scleritis, Scleroderma, Shiga-Toxin producing Escherichia Coli Hemolytic-Uremic Syndrome (STEC-HUS), Sjogren's syndrome, Small vessel vasculopathy, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenic purpura (TTP), Tolosa-Hunt syndrome, Transverse myelitis, Tubular autoimmune disorder, Ulcerative colitis, Undifferentiated connective tissue disease (UCTD), Uveitis, Vesiculobullous dermatosis, Vasculitis, Vitiligo, and Wegener's granulomatosis (also known as Granulomatosis with Polyangiitis (GPA)).

### Sterile Inflammation

Inflammatory indications may include sterile inflammation. Sterile inflammation is inflammation that occurs in response to stimuli other than infection. Sterile inflammation may be a common response to stress such as genomic stress, hypoxic stress, nutrient stress or endoplasmic reticulum stress caused by a physical, chemical, or metabolic noxious stimuli. Sterile inflammation may contribute to pathogenesis of many diseases such as, but not limited to, ischemia-induced injuries, rheumatoid arthritis, acute lung injuries, drug-induced liver injuries, inflammatory bowel diseases and/or other diseases, disorders or conditions. Mechanism of sterile inflammation and methods and compounds for treatment, prevention and/or delaying of symptoms of sterile inflammation may include any of those taught by Rubartelli et al. in Frontiers in Immunology, 2013, 4:398-99, Rock et al. in Annu Rev Immunol. 2010, 28:321-342 or in U.S. Pat. No. 8,101,586, the contents of each of which are herein incorporated by reference in their entirety. In some embodiments, complement inhibitor compounds and compositions of the present disclosure may be used to treat, prevent or delay development of sterile inflammation.

### Systemic Inflammatory Response (SIRS) and Sepsis

Inflammatory indications may include systemic inflammatory response syndrome (SIRS). SIRS is inflammation affecting the whole body. Where SIRS is caused by an infection, it is referred to as sepsis. SIRS may also be caused by non-infectious events such as trauma, injury, burns, ischemia, hemorrhage and/or other conditions. During sepsis and SIRS, complement activation leads to excessive generation of complement activation products which may cause multi organ failure (MOF) in subjects. In some embodiments, complement inhibitor compounds and compositions of the present disclosure may be used to treat and/or prevent SIRS. Complement inhibitor compounds and compositions may be used to control and/or balance complement activation for prevention and treatment of SIRS, sepsis and/or MOF. The methods of applying complement inhibitors to treat SIRS and sepsis may include those taught by Rittirsch et al. in Clin Dev Immunol, 2012, 962927, in U.S. publication No. US2013/0053302 or in U.S. Pat. No. 8,329,169, the contents of each of which are herein incorporated by reference in their entirety.

### Acute Respiratory Distress Syndrome (ARDS)

Inflammatory indications may include acute respiratory distress syndrome (ARDS). ARDS is a widespread inflammation of the lungs and may be caused by trauma, infection (e.g., sepsis), severe pneumonia and/or inhalation of harmful substances. ARDS is typically a severe, life-threatening complication. Studies suggest that neutrophils may contribute to development of ARDS by affecting the accumulation of polymorphonuclear cells in the injured pulmonary alveoli and interstitial tissue of the lungs. In some embodiments, complement inhibitor compounds and compositions of the present disclosure may be used to treat and/or prevent development of ARDS. Complement inhibitor compounds and compositions may be administered to reduce and/or prevent tissue factor production in alveolar neutrophils. Complement inhibitor compounds and compositions may further be used for treatment, prevention and/or delaying of ARDS, in some cases according to any of the methods taught in International publication No. WO2009/014633, the contents of which are herein incorporated by reference in their entirety.

### Periodontitis

Inflammatory indications may include periodontitis. Periodontitis is a widespread, chronic inflammation leading to the destruction of periodontal tissue which is the tissue supporting and surrounding the teeth. The condition also involves alveolar bone loss (bone that holds the teeth). Periodontitis may be caused by a lack of oral hygiene leading to accumulation of bacteria at the gum line, also known as dental plaque. Certain health conditions such as diabetes or malnutrition and/or habits such as smoking may increase the risk of periodontitis. Periodontitis may increase the risk of stroke, myocardial infarction, atherosclerosis, diabetes, osteoporosis, pre-term labor, as well as other health issues. Studies demonstrate a correlation between periodontitis and local complement activity. Periodontal bacteria may either inhibit or activate certain components of the complement cascade. In some embodiments, complement inhibitor compounds and compositions of the present disclosure may be used to treat or prevent development of periodontitis and/or associated conditions. Complement activation inhibitors and treatment methods may include any of those taught by Hajishengallis in Biochem Pharmacol. 2010, 15; 80(12): 1 and Lambris or in US publication No. US2013/0344082, the contents of each of which are herein incorporated by reference in their entirety.

### Dermatomyositis

Inflammatory indications may include dermatomyositis. Dermatomyositis is an inflammatory myopathy characterized by muscle weakness and chronic muscle inflammation. Dermatomyositis often begins with a skin rash that is associated concurrently or precedes muscle weakness. In some embodiments, complement inhibitor compounds and compositions of the present disclosure may be used to treat, prevent, or delay development of dermatomyositis.

### Rheumatoid Arthritis

Inflammatory indications may include rheumatoid arthritis. Rheumatoid arthritis is an autoimmune condition affecting the wrists and small joints of the hands. Typical symptoms include pain, stiffness of the joints, swelling, and feeling of warmth. Activated components of the complement system affect development of rheumatoid arthritis, as products of complement cascade mediate proinflammatory activities, such as vascular permeability and tone, leukocyte chemotaxis and the activation and lysis of multiple cell types (see Wang, et al., Proc. Natl. Acad. Sci., 1995; 92: 8955-8959). Wang et al. demonstrated that inhibition of C5 complement cascade in animals prevented the onset of arthritis and ameliorated established condition. Complement activation inhibitors and treatment methods may include any of those taught by Wang, et al., Proc. Natl. Acad. Sci., 1995; 92: 8955-8959, the contents of which are herein incorporated by reference in their entirety. In some embodiments, complement inhibitor compounds and compositions of the present disclosure may be used to treat or prevent development of rheumatoid arthritis.

### Asthma

Inflammatory indications may include asthma. Asthma is a chronic inflammation of the bronchial tubes, which are the airways allowing air to pass in and out of the lungs. The condition is characterized by narrowing, inflammation and hyperresponsiveness of the tubes. Typical symptoms include periods of wheezing, chest tightness, coughing and shortness of breath. Asthma the most common respiratory disorder. Complement proteins C3 and C5 are associated with many pathophysiological features of asthma, such as inflammatory cell infiltration, mucus secretion, increased vascular permeability, and smooth muscle cell contraction, and therefore it has been suggested that downregulation of complement activation may be used to treat, manage or prevent asthma. In some embodiments, complement inhibitor compounds and compositions of the present disclosure may be used to treat, prevent, or delay development of asthma. Complement activation inhibitors and treatment methods may include any of those taught by Khan et al., Respir Med. 2014 April; 108(4): 543-549, the contents of which are herein incorporated by reference in their entirety.

### Anaphylaxis

Inflammatory indications may include anaphylaxis. Anaphylaxis is a severe and potentially life-threatening allergic reaction. Anaphylaxis may lead to a shock characterized e.g. by sudden drop of blood pressure, narrowing of airways, breathing difficulties, rapid and weak pulse, a rash, nausea and vomiting. The cardiopulmonary collapse during anaphylaxis has been associated with complement activation and generation of C3a and C5a anaphylatoxins. Balzo et al. report animal studies indicating that complement activation markedly enhance cardiac dysfunction during anaphylaxis (Balzo et al., Circ Res. 1989 September; 65(3):847-57). Complement activation inhibitors and treatment methods may include any of those taught by Balzo et al., the contents of which are herein incorporated by reference in their entirety. In some embodiments, complement inhibitor compounds and compositions of the present disclosure may be used to treat, prevent, or delay development of anaphylaxis.

### Bowel Inflammation

Inflammatory indications may include inflammatory bowel disease (IBD). IBD is a reoccurring condition with periods of mild to severe inflammation or periods of remission. Common symptoms include diarrhea, fatigue and fever, abdominal pain, weight loss, reduced appetite and bloody stool. Types of IBD include ulcerative proctitis, dextran sulfate sodium colitis, proctosigmoitidis, left-sided colitis, panconlitis, acute severe ulcerative colitis. IBD, such as dextran sulfate sodium colitis and ulcerative colitis, have been associated with complement activity (Webb et al., Int J Med Pharm Case Reports. 2015; 4(5): 105-112 and Aomatsu et al., J Clin Biochem Nutr. 2013; 52(1):72-5). Complement activation inhibitors and treatment methods may include any of those taught by Webb et al. or Aomatsu et al, the contents of each of which are herein incorporated by reference in their entirety. In some embodiments, complement inhibitor compounds and compositions of the present disclosure may be used to treat, prevent, or delay development of IBD.

### Systemic Inflammation During Cardiopulmonary Bypass

Inflammatory indications may include inflammatory response induced by cardiopulmonary bypass (CBP). CBP is a technique used during surgery to take over the function of heart and lungs to maintain blood circulation and oxygen concentration of the blood. CBD provokes a systemic inflammatory response that may lead to complications of the surgical patients. The suggested cause may be due to contact activation of blood with artificial surfaces during extracorporeal circulation. The inflammation response may lead to SIRS and be life-threatening.

Complement activation has been associated with the inflammatory response induced by CBP. Studies have suggested that terminal components C5a and C5b-9 directly contribute to platelet and neutrophil activation during the extracorporeal blood circulation and C5 has been identified as a therapeutic site for prevention and treatment of inflammatory response induced by CBP (Rinder et al. J Clin Invest. 1995; 96(3): 1564-1572). Complement activation inhibitors and treatment methods may include any of those taught by Rinder et al. J Clin Invest. 1995; 96(3): 1564-1572, the contents of which are herein incorporated by reference in their entirety. In some embodiments, complement inhibitor compounds and compositions of the present disclosure may be used to treat, prevent, or delay development of inflammatory response induced by CBP.

### Rejection in Organ or Tissue Transplant

Inflammatory indications may include immune rejection of transplants. Transplants may be organs (e.g. heart, kidneys, liver, lungs, intestine, thymus and pancreas) or tissues (e.g. bones, tendons, skin, cornea, veins). Different types of transplants include autograft (transplanting patient's own tissue), allograft (transplant between two members of the same species) or xenograft (transplant between members of different species, e.g. from an animal to a human). Complications after organ transplant arise as the recipient's immune system attacks the transplanted tissue. The rejection may be hyperacute referring to a reaction occurring within few minutes after the transplant is performed, and typically occurs when the antigens are unmatched. Acute rejection occurs within a week or few months after transplant. Some rejections are chronic and take place over many years.

Transplant rejection and related inflammation has been associated with complement system. The complement cascade is relevant to transplantation in a number of ways, e.g. as an effector mechanism of antibody-initiated allograft injury, promotion of ischemia-reperfusion injury, and formation and function of alloantibodies (Sheen and Heeger, Curr Opin Organ Transplant. 2015; 20(4):468-75). Therapy targeting complement has been suggested to have significance for the survival and health of transplant patients As an example, studies have shown that C5 blockage of C5 with eculizumab reduces the incidence of early antibody-mediated rejection (AMR) of organ allografts (Stegall et al., Nature Reviews Nephrology 8(11):670-8, 2012) and inhibition of C5 may prevent acute cardiac tissue injury in an ex vivo model of pig-to-human xenotransplantation (Kroshus et al, Transplantation. 1995, 15; 60(11):1194-202.) Complement activation inhibitors and treatment methods may include any of those taught by Stegall et al., Nature Reviews Nephrology 8(11):670-8, 2012 and Kroshus et al, Transplantation. 1995, 15; 60(11):1194-202, and (Sheen and Heeger, Curr Opin Organ Transplant. 2015; 20(4):468-75, the contents of each of which are herein incorporated by reference in their entirety. In some embodiments, complement inhibitor compounds and/or compositions of the present disclosure may be used to treat subjects with or receiving transplanted organs or tissues.

### Wounds and Injuries

Therapeutic indications that may be addressed with compounds and/or compositions of the present disclosure may include wounds and injuries. As used herein, the term “injury” typically refers to physical trauma, but may include localized infection or disease processes. Injuries may be characterized by harm, damage or destruction caused by external events affecting body parts and/or organs. Non-limiting examples of injuries include head trauma and crush injuries. Wounds are associated with cuts, blows, burns and/or other impacts to the skin, leaving the skin broken or damaged. Wounds and injuries may include complement-related indications. Wounds and injuries are often acute but if not healed properly they may lead to chronic complications and/or inflammation. In some embodiments, complement inhibitor compounds and compositions of the present disclosure may be used to treat and/or promote healing of different types of wounds and/or injuries.

### Wounds and Burn Wounds

In some embodiments, complement inhibitor compounds and compositions of the present disclosure may be used to treat and/or to promote healing of wounds. Healthy skin provides a waterproof, protective barrier against pathogens and other environmental effectors. The skin also controls body temperature and fluid evaporation. When skin is wounded these functions are disrupted making skin healing challenging. Wounding initiates a set of physiological processes related to the immune system that repair and regenerate tissue. Complement activation is one of these processes. Complement activation studies have identified several complement components involved with wound healing as taught by van de Goot et al. in J Burn Care Res 2009, 30:274-280 and Cazander et al. Clin Dev Immunol, 2012, 2012:534291, the contents of each of which are herein incorporated by reference in their entirety. In some cases, complement activation may be excessive, causing cell death and enhanced inflammation (leading to impaired wound healing and chronic wounds). In some cases, complement inhibitor compounds and compositions may be used to reduce or eliminate such complement activation to promote wound healing. Treatment with complement inhibitor compounds and compositions may be carried out according to any of the methods for treating wounds disclosed in International Publication No. WO2012/174055, the contents of which are herein incorporated by reference in their entirety.

### Head Trauma

Wounds and/or injuries may include head trauma. Head traumas include injuries to the scalp, the skull or the brain. Examples of head trauma include, but are not limited to concussions, contusions, skull fracture, traumatic brain injuries and/or other injuries. Head traumas may be minor or severe. In some cases, head trauma may lead to long term physical and/or mental complications or death. Studies indicate that head traumas may induce improper intracranial complement cascade activation, which may lead to local inflammatory responses contributing to secondary brain damage by development of brain edema and/or neuronal death (Stahel et al. in Brain Research Reviews, 1998, 27: 243-56, the contents of which are herein incorporated by reference in their entirety). In some embodiments, complement inhibitor compounds and compositions of the present disclosure may be used to treat head trauma and/or prevent or delay development of diseases, disorders, and/or conditions associated with head trauma. In some embodiments, complement inhibitor compounds and compositions may be used to treat, prevent, reduce, or delay development of secondary complications of head trauma. Methods of using complement inhibitor compounds and compositions to control complement cascade activation in head trauma may include any of those taught by Holers et al. in U.S. Pat. No. 8,911,733, the contents of which are herein incorporated by reference in their entirety.

### Crush Injury

Wounds and/or injuries may include crush injuries. Crush injuries are injuries caused by a force or a pressure put on the body causing bleeding, bruising, fractures, nerve injuries, wounds and/or other damages to the body. In some embodiments, complement inhibitor compounds and compositions of the present disclosure may be used to treat and/or promote healing of crush injuries. Treatment may be used to reduce complement activation following crush injuries, thereby promoting healing after crush injuries (e.g., by promoting nerve regeneration, promoting fracture healing, preventing or treating inflammation, and/or other related complications). Complement inhibitor compounds and compositions may be used to promote healing according to any of the methods taught in U.S. Pat. No. 8,703,136; International Publication Nos. WO2012/162215; WO2012/174055; or US publication No. US2006/0270590, the contents of each of which are herein incorporated by reference in their entirety.

### Autoimmune Indications

Therapeutic indications addressed with compounds and/or compositions of the present disclosure may include autoimmune indications. As used herein, the term “autoimmune indication” refers to any therapeutic indication relating to immune targeting of a subject's tissues and/or substances by the subject's own immune system. Autoimmune indications may include complement-related indications. Autoimmune indications may involve certain tissues or organs of the body. The immune system may be divided into innate and adaptive systems, referring to nonspecific immediate defense mechanisms and more complex antigen-specific systems, respectively. The complement system is part of the innate immune system, recognizing and eliminating pathogens. Additionally, complement proteins may modulate adaptive immunity, connecting innate and adaptive responses. Complement inhibitor compounds and compositions of the present disclosure may be used to modulate complement in the treatment and/or prevention of autoimmune diseases. In some cases, such compounds and compositions may be used according to the methods presented in Ballanti et al. Immunol Res (2013) 56:477-491, the contents of which are herein incorporated by reference in their entirety. In some embodiments, autoimmune indications include myasthenia gravis.

### Anti-Phospholipid Syndrome (APS) and Catastrophic Anti-Phospholipid Syndrome (CAPS)

Autoimmune indications may include anti-phospholipid syndrome (APS). APS is an autoimmune condition caused by anti-phospholipid antibodies that cause the blood to clot. APS may lead to recurrent venous or arterial thrombosis in organs, and complications in placental circulations causing pregnancy-related complications such as miscarriage, still birth, preeclampsia, premature birth and/or other complications. Catastrophic anti-phospholipid syndrome (CAPS) is an extreme and acute version of a similar condition leading to occlusion of veins in several organs simultaneously. Studies suggest that complement activation may contribute to APS-related complications including pregnancy-related complications, thrombotic (clotting) complications, and vascular complications. In some embodiments, complement inhibitor compounds and compositions of the present disclosure may be used to treat, prevent, or delay development of APS and/or APS-related complications. In some embodiments, complement inhibitor compounds and compositions of the present disclosure may be used to prevent and/or treat APS by complement activation control. In some cases, complement inhibitor compounds and compositions may be used to treat APS and/or APS-related complications according to the methods taught by Salmon et al. Ann Rheum Dis 2002; 61(Suppl II):ii46-ii50 and Mackworth-Young in Clin Exp Immunol 2004, 136:393-401, the contents of which are herein incorporated by reference in their entirety.

### Cold Agglutinin Disease

Autoimmune indications may include cold agglutinin disease (CAD), also referred to as cold agglutinin-mediated hemolysis. CAD is an autoimmune disease resulting from a high concentration of IgM antibodies interacting with red blood cells at low range body temperatures (Engelhardt et al. Blood, 2002, 100(5):1922-23). CAD may lead to conditions such as anemia, fatigue, dyspnea, hemoglobinuria and/or acrocyanosis. CAD is related to robust complement activation and studies have shown that CAD may be treated with complement inhibitor therapies. In some embodiments, complement inhibitor compounds and compositions of the present disclosure may be used to treat, prevent, or delay development of CAD. Such uses may treat CAD by inhibiting complement activity. In some cases, complement inhibitor compounds and compositions may be used to treat CAD according to the methods taught by Roth et al in Blood, 2009, 113:3885-86 or in International publication No. WO2012/139081, the contents of each of which are herein incorporated by reference in their entirety.

### Dermatological Diseases

Autoimmune indications may include dermatological disease. Skin has a role in a spectrum of immunological reactions and are associated with abnormal or overactivated complement protein functions. Autoimmune mechanisms with autoantibodies and cytotoxic functions of the complement affect epidermal or vascular cells causing tissue damage and skin inflammation (Palenius and Meri, Front Med (Lausanne). 2015; 2: 3). Dermatological diseases associated with autoimmune and complement abnormality include, but are not limited to, hereditary and acquired angioedema, autoimmune urticarial (hives), systemic lupus erythematosus, vasculitis syndromes and urticarial vasculitis, bullous skin diseases (e.g. pemphigus, bullous pemphigioid, mucous membrane pemphigoid, epidermolysis bullosa acquisita, dermatitis herpetiformis, pemphigoides festationis), and partial lipodustrophy. In some cases, complement inhibitor compounds and compositions may be used to treat autoimmune dermatological diseases according to the methods taught by Palenius and Meri, Front Med (Lausanne). 2015; 2: 3, the contents of which are herein incorporated by reference in their entirety. In some embodiments, complement inhibitor compounds and compositions of the present disclosure may be used to treat, prevent, or delay development of dermatological diseases.

### Pulmonary Indications

Therapeutic indications addressed with compounds and/or compositions of the present disclosure may include pulmonary indications. As used herein, the term “pulmonary indication” refers to any therapeutic indication related to the lungs and/or related airways. Pulmonary indications may include complement-related indications. Pulmonary indications may include, but are not limited to, asthma, pulmonary fibrosis, chronic obstructive pulmonary disease (COPD), and acute respiratory distress syndrome. In some embodiments, complement inhibitor compounds and compositions of the present disclosure may be used to treat, prevent, or delay development of pulmonary indications.

### Chronic Obstructive Pulmonary Disease (COPD)

Pulmonary indications may include chronic obstructive pulmonary disease (COPD). COPD refers to a class of disorders related to progressive lung dysfunction. They are most often characterized by breathlessness. Complement dysfunction has been indicated as a contributor to some pulmonary indications related to COPD (Pandya, P. H. et al. 2013. Translational Review. 51(4): 467-73, the contents of which are herein incorporated by reference in their entirety). In some embodiments, complement inhibitor compounds and compositions of the present disclosure may be used to treat, prevent, or delay development of COPD.

### Cardiovascular Indications

Therapeutic indications addressed with compounds and/or compositions of the present disclosure may include cardiovascular indications. As used herein, the term “cardiovascular indication” refers to any therapeutic indication relating to the heart and/or vasculature. Cardiovascular indications may include complement-related indications. Cardiovascular indications may include, but are not limited to, atherosclerosis, myocardial infarction, stroke, vasculitis, trauma and conditions arising from cardiovascular intervention (including, but not limited to cardiac bypass surgery, arterial grafting and angioplasty). In some embodiments, complement inhibitor compounds and compositions of the present disclosure may be used to treat, prevent, or delay development of cardiovascular indications.

Vascular indications are cardiovascular indications related to blood vessels (e.g., arteries, veins, and capillaries). Such indications may affect blood circulation, blood pressure, blood flow, organ function, and/or other bodily functions. In some embodiments, complement inhibitor compounds and compositions of the present disclosure may be used to treat, prevent, or delay development of vascular indications.

### Coagulation

In some embodiments, cardiovascular indications include therapeutic indications associated with coagulation, the coagulation cascade, and/or coagulation cascade components. Historically, the complement activation pathway was viewed separately from the coagulation cascade; however, interplay between these two systems has more recently been appreciated. Coagulation and complement are coordinately activated in an overlapping spatiotemporal manner in response to common pathophysiologic stimuli to maintain homeostasis. Disease may emerge with unchecked activation of the innate immune and coagulation responses. Examples include, for example, atherosclerosis, stroke, coronary heart disease, diabetes, ischemia-reperfusion injury, trauma, paroxysmal nocturnal hemoglobinuria, age-related macular degeneration, and atypical hemolytic-uremic syndrome.

Several molecular links between complement and coagulation are currently appreciated. For example, thrombin was found to promote complement activation by cleaving C5 (Huber-Lang, et al., 2006. Nature Med. 12(6):682-687; the contents of which are herein incorporated by reference in their entirety). While thrombin is capable of cleaving C5 at R751 (yielding C5a and C5b), it more efficiently cleaves C5 at a highly conserved R947 site, generating C5_{T} and C5b_{T} intermediates. C5b_{T} interacts with other complement proteins to form the C5b_{T}-9 membrane attack complex with significantly more lytic activity than with C5b-9 (Krisinger, et al., (2014). Blood. 120(8):1717-1725).

Complement may be activated by additional components of the coagulation and/or inflammation cascades. For example, other serine proteases with slightly different substrate specificity may act in a similar way. Huber-Lang et al. (2006) showed that thrombin not only cleaved C5 but also generated C3a in vitro when incubated with native C3 (Huber-Lang, et al., 2006. Nature Med. 12(6):682-687). Similarly, other components of the coagulation pathway, such as FXa, FXIa and plasmin, have been found to cleave both C5 and C3.

Specifically, in a mechanism similar to the one observed via thrombin activation, it has been observed that plasmin, FXa, FIXa and FXIa are able to cleave C5 to generate C5a and C5b [Amara, et al., (2010). J. Immunol. 185:5628-5636; Amara, et al., (2008) Current Topics in Complement II, J. D. Lambris (ed.), pp. 71-79]. The anaphylatoxins produced were found to be biologically active as shown by a dose-dependent chemotactic response of neutrophils and HMC-1 cells, respectively. Plasmin-induced cleavage activity could be dose-dependently blocked by the serine protease inhibitor aprotinin and leupeptine. These findings suggest that various serine proteases belonging to the coagulation system are able to activate the complement cascade independently of the established pathways. Moreover, functional C5a and C3a are generated (as detected by immunoblotting and ELISA), both of which are known to be crucially involved in the inflammatory response.

In some embodiments, compounds and compositions of the present disclosure may be used to treat cardiovascular indications related to coagulation, the coagulation cascade, and/or coagulation cascade components. The coagulation cascade components may include, but are not limited to, tissue factor, thrombin, FXa, FIXa, FXIa, plasmin, or other coagulation proteases. Compounds and/or compositions of the present disclosure may be used to treat complement activity and/or coagulation (e.g., thrombosis) associated with such cardiovascular indications.

### Thrombotic Microangiopathy (7MA)

Vascular indications may include thrombotic microangiopathy (TMA) and associated diseases. Microangiopathies affect small blood vessels (capillaries) of the body causing capillary walls to become thick, weak, and prone to bleeding and slow blood circulation. TMAs tend to lead to the development of vascular thrombi, endothelial cell damage, thrombocytopenia, and hemolysis. Organs such as the brain, kidney, muscles, gastrointestinal system, skin, and lungs may be affected. TMAs may arise from medical operations and/or conditions that include, but are not limited to, hematopoietic stem cell transplantation (HSCT), renal disorders, diabetes and/or other conditions. TMAs may be caused by underlying complement system dysfunction, as described by Meri et al. in European Journal of Internal Medicine, 2013, 24: 496-502, the contents of which are herein incorporated by reference in their entirety. Generally, TMAs may result from increased levels of certain complement components leading to thrombosis. In some cases, this may be caused by mutations in complement proteins or related enzymes. Resulting complement dysfunction may lead to complement targeting of endothelial cells and platelets leading to increased thrombosis. In some embodiments, TMAs may be prevented and/or treated with complement inhibitor compounds and compositions of the present disclosure. In some cases, methods of treating TMAs with complement inhibitor compounds and compositions may be carried out according to those described in US publication Nos. US2012/0225056 or US2013/0246083, the contents of each of which are herein incorporated by reference in their entirety.

### Disseminated Intravascular Coagulation (DIC)

Vascular indications may include disseminated intravascular coagulation (DIC). DIC is a pathological condition where the clotting cascade in blood is widely activated and results in formation of blood clots especially in the capillaries. DIC may lead to an obstructed blood flow of tissues and may eventually damage organs. Additionally, DIC affects the normal process of blood clotting that may lead to severe bleeding. Complement inhibitor compounds and compositions of the present disclosure may be used to treat, prevent or reduce the severity of DIC by modulating complement activity. In some cases, complement inhibitor compounds and compositions may be used according to any of the methods of DIC treatment taught in U.S. Pat. No. 8,652,477, the contents of which are herein incorporated by reference in their entirety.

### Vasculitis

Vascular indications may include vasculitis. Generally, vasculitis is a disorder related to inflammation of blood vessels, including veins and arteries, characterized by white blood cells attacking tissues and causing swelling of the blood vessels. Vasculitis may be associated with an infection, such as in Rocky Mountain spotted fever, or autoimmunity. An example of autoimmunity associated vasculitis is Anti-Neutrophil Cytoplasmic Autoantibody (ANCA) vasculitis. ANCA vasculitis is caused by abnormal antibodies attacking the body's own cells and tissues. ANCAs attack the cytoplasm of certain white blood cells and neutrophils, causing them to attack the walls of the vessels in certain organs and tissues of the body. ANCA vasculitis may affect skin, lungs, eyes and/or kidney. Studies suggest that ANCA disease activates an alternative complement pathway and generates certain complement components that create an inflammation amplification loop resulting in a vascular injury (Jennette et al. 2013, Semin Nephrol. 33(6): 557-64, the contents of which are herein incorporated by reference in their entirety). In some embodiments, complement inhibitor compounds and compositions of the present disclosure may be used to prevent and/or treat vasculitis. In some cases, complement inhibitor compounds and compositions may be used to prevent and/or treat ANCA vasculitis by inhibiting complement activation.

### Neurological Indications

Therapeutic indications addressed with compounds and/or compositions of the present disclosure may include neurological indications. As used herein, the term “neurological indication” refers to any therapeutic indication relating to the nervous system. Neurological indications may include complement-related indications. Neurological indications may include neurodegeneration. Neurodegeneration generally relates to a loss of structure or function of neurons, including death of neurons. In some embodiments, complement inhibitor compounds and compositions of the present disclosure may be used to treat, prevent, or delay development of neurological indications, including, but not limited to neurodegenerative diseases and related disorders. Treatment may include inhibiting the effect of complement activity on neuronal cells using compounds and compositions of the present disclosure. Neurodegenerative related disorders include, but are not limited to, Amyelotrophic Lateral Sclerosis (ALS), Multiple Sclerosis (MS), Parkinson's disease, Alzheimer's disease, and Lewy body dementia. In some embodiments, complement-related neurological indications include myasthenia gravis.

### Amyotrophic Lateral Sclerosis (ALS)

Neurological indications may include ALS. ALS is a fatal motor neuron disease characterized by the degeneration of spinal cord neurons, brainstems and motor cortex. ALS causes loss of muscle strength leading eventually to a respiratory failure. Complement dysfunction may contribute to ALS, and therefore ALS may be prevented, treated and/or the symptoms may be reduced by therapy with complement inhibitor compounds and compositions targeting complement activity. In some embodiments, complement inhibitor compounds and compositions of the present disclosure may be used to treat, prevent, or delay development of ALS and/or promote nerve regeneration. In some cases, complement inhibitor compounds and compositions may be used as complement inhibitors according to any of the methods taught in US publication No. US2014/0234275 or US2010/0143344, the contents of each of which are herein incorporated by reference in their entirety.

### Alzheimer's Disease

Neurological indications may include Alzheimer's disease. Alzheimer's disease is a chronic neurodegenerative disease with symptoms that may include disorientation, memory loss, mood swings, behavioral problems and eventually loss of bodily functions. Alzheimer's disease is thought to be caused by extracellular brain deposits of amyloid that are associated with inflammation-related proteins such as complement proteins (Sjoberg et al. 2009. Trends in Immunology. 30(2): 83-90, the contents of which are herein incorporated by reference in their entirety). In some embodiments, complement inhibitor compounds and compositions of the present disclosure may be used to treat, prevent, or delay development of Alzheimer's disease by controlling complement activity. In some cases, complement inhibitor compounds and compositions may be used according to any of the Alzheimer's treatment methods taught in US publication No. US2014/0234275, the contents of which are herein incorporated by reference in their entirety.

### Multiple Sclerosis and Neuromyelitis Optica

Neurological indications may include multiple sclerosis (MS) or neuromyelitis optica (NMO). MS is an inflammatory condition affecting the central nervous system as the immune system launches an attack against the body's own tissues, and in particular against nerve-insulating myelin. The condition may be triggered by an unknown environmental agent, such as a virus. MS is progressive and eventually results in disruption of the communication between the brain and other parts of the body. Typical early symptoms include blurred vision, partial blindness, muscle weakness, difficulties in coordination and balance, impaired movement, pain and speech impediments. NMO (also known as Devic's disease) is an inflammatory demyelinating disease affecting the optic nerves and spinal cord as the immune system attacks the astrocytes. NMO is sometimes considered as a variant of MS. Typical symptoms of NMO include muscle weakness of the legs or paralysis, loss of senses (e.g. blindness) and dysfunctions of the bladder and bowel.

MS and NMO have been associated with complement component regulation e.g. by pathological and animal model studies (Ingram et al., Clin Exp Immunol. 2009 February; 155(2): 128-139). In the central nervous system glial cells and neurons produce the majority of complement proteins and the expression is increased in response to inflammation. In some embodiments, complement inhibitor compounds and compositions of the present disclosure may be used to treat, prevent, or delay development of MS or NMO. Treatment methods may include any of those taught by Ingram et al., Clin Exp Immunol. 2009 February; 155(2): 128-139, the contents of which are herein incorporated by reference in their entirety.

### Myasthenia Gravis

Neurological indications may include myasthenia gravis. Myasthenia gravis (MG) is a rare complement-mediated autoimmune disease characterized by the production of autoantibodies targeting proteins that are critical for the normal transmission of chemical or neurotransmitter signals from nerves to muscles, e.g., acetylcholine receptor (AChR) proteins. The presence of AChR autoantibodies in patient samples can be used as an indicator of disease. As used herein, the term “MG” embraces any form of MG. While about 15% of patients have symptoms that are confined to ocular muscles, the majority of patients experience generalized myasthenia gravis. As used herein, the term “generalized myasthenia gravis” or “gMG” refers to MG that affects multiple muscle groups throughout the body. Although the prognosis of MG is generally benign, 10% to 15% of patients have refractory MG. As used herein, the term “refractory MG” or “rMG” refers to MG where disease control either cannot be achieved with current therapies, or results in severe side effects of immunosuppressive therapy. This severe form of MG affects approximately 9,000 individuals in the United States.

Patients with MG present with muscle weakness that characteristically becomes more severe with repeated use and recovers with rest. Muscle weakness can be localized to specific muscles, such as those responsible for eye movements, but often progresses to more diffuse muscle weakness. MG may even become life-threatening when muscle weakness involves the diaphragm and the other chest wall muscles responsible for breathing. This is the most feared complication of MG, known as myasthenic crisis or MG crisis, and requires hospitalization, intubation, and mechanical ventilation. Approximately 15% to 20% of patients with gMG experience a myasthenic crisis within two years of diagnosis.

The most common target of autoantibodies in MG is the acetylcholine receptor, or AChR, located at the neuromuscular junction, the point at which a motor neuron transmits signals to a skeletal muscle fiber. Current therapies for gMG focus on either augmenting the AChR signal or nonspecifically suppressing the autoimmune response. First-line therapy for symptomatic gMG is treatment with acetylcholinesterase inhibitors such as pyridostigmine, which is the only approved therapy for MG. Although sometimes adequate for control of mild ocular symptoms, pyridostigmine monotherapy is usually insufficient for the treatment of generalized weakness, and dosing of this therapy may be limited by cholinergic side effects. Therefore, in patients who remain symptomatic despite pyridostigmine therapy, corticosteroids with or without systemic immunosuppressives are indicated (Sanders D B, et al. 2016. Neurology. 87(4):419-25). Immunosuppressives used in gMG include azathioprine, cyclosporine, mycophenolate mofetil, methotrexate, tacrolimus, cyclophosphamide, and rituximab. To date, efficacy data for these agents are sparse and no steroidal or immunosuppressive therapy has been approved for the treatment of gMG. Moreover, all of these agents are associated with well-documented long-term toxicities. Surgical removal of the thymus may be recommended in patients with nonthymomatous gMG and moderate to severe symptoms in an effort to reduce the production of AChR autoantibodies (Wolfe G I, et al. 2016. N Engl J Med. 375(6):511-22). Intravenous (IV) immunoglobulin and plasma exchange are usually restricted to short-term use in patients with myasthenic crisis or life-threatening signs such as respiratory insufficiency or dysphagia (Sanders et al., 2016).

There is substantial evidence that supports the role of terminal complement cascade in the pathogenesis of AChR autoantibody-positive gMG. Results from animal models of experimental autoimmune MG have demonstrated that autoantibody immune complex formation at the neuromuscular junction triggers activation of the classical complement pathway, resulting in local activation of C3 and deposition of the membrane attack complex (MAC) at the neuromuscular junction, resulting in loss of signal transduction and eventual muscle weakness (Kusner L L, et al., 2012. Ann N Y Acad Sci. 1274(1):127-32).

Binding of anti-AChR autoantibodies to the muscle endplate results in activation of the classical complement cascade and deposition of MAC on the post-synaptic muscle fiber leading to local damage to the muscle membrane, and reduced responsiveness of the muscle to stimulation by the neuron.

In some embodiments, complement inhibitor compounds and compositions of the present disclosure may be used to treat, prevent, or delay development of MG (e.g., gMG and/or rMG). Inhibition of complement activity may be used to block complement-mediated damage resulting from MG (e.g., gMG and/or rMG).

### Kidney-Related Indications

Therapeutic indications addressed with compounds and/or compositions of the present disclosure may include kidney-related indications. As used herein, the term “kidney-related indication” refers to any therapeutic indication involving kidneys. Kidney-related indications may include complement-related indications. Kidneys are organs responsible for removing metabolic waste products from the blood stream. Kidneys regulate blood pressure, the urinary system, and homeostatic functions and are therefore essential for a variety of bodily functions. Kidneys may be more seriously affected by inflammation (as compared to other organs) due to unique structural features and exposure to blood. Kidneys also produce their own complement proteins which may be activated upon infection, kidney disease, and renal transplantations. In some embodiments, complement inhibitor compounds and compositions of the present disclosure may be used to treat, prevent, or delay development of kidney-related indications, in some cases by inhibiting complement activity. In some cases, complement inhibitor compounds and compositions may be used to treat kidney-related indications according to the methods taught by Quigg, J Immunol 2003; 171:3319-24, the contents of which are herein incorporated by reference in their entirety.

Atypical Hemolytic Uremic Syndrome (aHUS)

Kidney-related indications may include atypical hemolytic uremic syndrome (aHUS). aHUS belongs to the spectrum of thrombotic microangiopathies. aHUS is a condition causing abnormal blood clots formation in small blood vessels of the kidneys. The condition is commonly characterized by hemolytic anemia, thrombocytopenia and kidney failure, and leads to end-stage renal disease (ESRD) in about half of all cases. aHUS has been associated with abnormalities of the alternative pathway of the complement system and may be caused by a genetic mutation in one of the genes that lead to increased activation of the alternative pathway. (Verhave et al., Nephrol Dial Transplant. 2014; 29 Suppl 4:iv131-41 and International Publication WO 2016/138520). aHUS may be treated by inhibitors that control the alternative pathway of complement activation, including C5 activation. In some embodiments, complement inhibitor compounds and compositions of the present disclosure may be used to treat, prevent or delay development of aHUS. Methods and compositions for preventing and/or treating aHUS by complement inhibition may include any of those taught by Verhave et al. in Nephrol Dial Transplant. 2014; 29 Suppl 4:iv131-41 or International Publication WO 2016/138520, the contents of each of which are herein incorporated by reference in their entirety.

### Lupus Nephritis

Kidney-related indications may include lupus nephritis. Lupus nephritis is a kidney inflammation caused by an autoimmune disease called systemic lupus erythematosus (SLE). Symptoms of lupus nephritis include high blood pressure; foamy urine; swelling of the legs, the feet, the hands, or the face; joint pain; muscle pain; fever; and rash. In some embodiments, complement inhibitor compounds and compositions of the present disclosure may be used to treat, prevent, or delay development of lupus nephritis, in some cases through complement activity inhibition. Related methods may include any of those taught in US publication No. US2013/0345257 or U.S. Pat. No. 8,377,437, the contents of each of which are herein incorporated by reference in their entirety.

### Membranous Glomerulonephritis (MGN)

Kidney-related indications may include membranous glomerulonephritis (MGN). MGN is a disorder of the kidney that may lead to inflammation and structural changes. MGN is caused by antibodies binding to a soluble antigen in kidney capillaries (glomerulus). MGN may affect kidney functions, such as filtering fluids and may lead to kidney failure. In some embodiments, complement inhibitor compounds and compositions of the present disclosure may be used to treat, prevent, or delay development of MGN, including by inhibiting complement activity. Related treatment methods may include any of those taught in U.S. publication No. US2010/0015139 or in International publication No. WO2000/021559, the contents of each of which are herein incorporated by reference in their entirety.

### Hemodialysis Complications

Kidney-related indications may include hemodialysis complications. Hemodialysis is a medical procedure used to maintain kidney function in subjects with kidney failure. In hemodialysis, the removal of waste products such as creatinine, urea, and free water from blood is performed externally. A common complication of hemodialysis treatment is chronic inflammation caused by contact between blood and the dialysis membrane. Another common complication is thrombosis referring to a formation of blood clots that obstructs the blood circulation. Studies have suggested that these complications are related to complement activation. Hemodialysis may be combined with complement inhibitor therapy to provide means of controlling inflammatory responses and pathologies and/or preventing or treating thrombosis in subjects going through hemodialysis due to kidney failure. In some embodiments, complement inhibitor compounds and compositions of the present disclosure may be used to treat, prevent, or delay development of hemodialysis complications, including by inhibiting complement activation. Related methods for treatment of hemodialysis complications may include any of those taught by DeAngelis et al in Immunobiology, 2012, 217(11): 1097-1105 or by Kourtzelis et al. Blood, 2010, 116(4):631-639, the contents of each of which are herein incorporated by reference in their entirety.

### IgA Nephropathy

Kidney-related indications may include IgA nephropathy. IgA nephropathy is the most common cause of glomerulonephritis, affecting 25 in every one million per year. The disease is characterized by mesangial deposits of IgA and complement components in the glomeruli. In some embodiments, complement inhibitor compounds and compositions of the present disclosure may be used to treat, prevent, or delay development of IgA nephropathy by inhibiting the activation of certain complement components. Compounds and compositions of the invention may be used according to methods of preventing and/or treating IgA nephropathy by complement inhibition taught by Maillard N et al., in J of Am Soc Neph (2015) 26(7):1503-1512, the contents of each of which are herein incorporated by reference in their entirety

### Dense Deposit Disease/Membranoproliferative Glomerulonephritis Type II/C3 Glomerulopathy

Kidney-related indications may include dense deposit disease, membranoproliferative glomerulonephritis type II, and C3 glomerulopathy. Dense deposit disease (DDD) is a complement-related indication that involves kidney disorder. DDD may include proteinuria, hematuria, reduced amounts of urine, low levels of protein in the blood, and swelling in many areas of the body. DDD can be caused by mutations in the C3 and CFH genes; by both genetic risk factors and environmental triggers; or by the presence of autoantibodies blocking the activity of proteins needed for the body's immune response. In some embodiments, complement inhibitor compounds and compositions of the present disclosure may be used to treat, prevent, or delay development of DDD. Such uses may include reducing and/or blocking complement alternative pathway activity. Such methods may prevent glomerular C3 deposition.

### Focal-Segmental Glomerulosclerosis

Kidney-related indications may include focal-segmental glomerulosclerosis. Focal-segmental glomerulosclerosis (FSGS) is a common cause of glomerular disease in children and adults and most commonly presents as severe nephrotic syndrome. Diagnosis of FSGS is made based on histopathological findings and exclusion of other diagnoses common in nephrotic syndrome. Many patients will have substantial deposition of IgM and C3 in sclerotic regions on biopsy. Additionally, biomarkers for complement activation (factor B fragments, C4a, soluble MAC) have been detected in plasma and urine from patients with FSGS, with levels of Ba and Bb correlating with disease severity (J. Thurman et al, PLOSone, 2015). In some embodiments, complement inhibitor compounds and compositions of the present disclosure may be used to treat, prevent, or delay development of FSGS.

### Diabetes-Related Indications

Therapeutic indications addressed with compounds and/or compositions of the present disclosure may include diabetes-related indications. As used herein, the term “diabetes-related indication” refers to any therapeutic indication resulting from or relating to elevated blood sugar. Diabetes-related indications may include complement-related indications. Diabetes-related indications may occur as a result of organ and/or tissue exposure to prolonged hyperglycemia. Prolonged hyperglycemia can result in glycation inactivation of the membrane-associated complement regulatory protein CD59, leaving certain cells and tissues susceptible to complement attack (P. Ghosh et al, 2015. Endocrine Reviews, 36 (3), 2015). Complement-mediated complications from diabetes may include, but are not limited to, diabetic neuropathy, diabetic nephropathy, diabetic cardiovascular disease, and complications resulting from gestational diabetes such as high or low birth weight and resulting complications. In some embodiments, complement inhibitor compounds and compositions of the present disclosure may be used to treat, prevent, or delay development of diabetes-related indications. Such uses may include addressing diabetes-related indications through complement activity inhibition.

### Ocular Indications

Therapeutic indications addressed with compounds and/or compositions of the present disclosure may include ocular indications. As used herein, the term “ocular indication” refers to any therapeutic indication relating to the eye. Ocular indications may include complement-related indications. In a healthy eye the complement system is activated at a low level and is continuously regulated by membrane-bound and soluble intraocular proteins that protect against pathogens. Therefore, the activation of complement plays an important role in several complications related to the eye and controlling complement activation may be used to treat such diseases. In some embodiments, complement inhibitor compounds and compositions of the present disclosure may be used to treat, prevent, or delay development of ocular indications, including by inhibiting complement activity. Related treatment methods may include any of those taught by Jha et al. in Mol Immunol. 2007; 44(16): 3901-3908 or in U.S. Pat. No. 8,753,625, the contents of each of which are herein incorporated by reference in their entirety.

Ocular indications may include, but are not limited to, age-related macular degeneration, allergic and giant papillary conjunctivitis, Behcet's disease, choroidal inflammation, complications related to intraocular surgery, corneal transplant rejection, corneal ulcers, cytomegalovirus retinitis, dry eye syndrome, endophthalmitis, Fuch's disease, Glaucoma, immune complex vasculitis, inflammatory conjunctivitis, ischemic retinal disease, keratitis, macular edema, ocular parasitic infestation/migration, retinitis pigmentosa, scleritis, Stargardt disease, subretinal fibrosis, uveitis, vitreo-retinal inflammation, and Vogt-Koyanagi-Harada disease.

### Age-Related Macular Degeneration (AMD)

Ocular indications may include age-related macular degeneration (AMD). AMD is a chronic ocular disease causing blurred central vision, blind spots in central vision, and/or eventual loss of central vision. Central vision affects ability to read, drive a vehicle and/or recognize faces. AMD is generally divided into two types, non-exudative (dry) and exudative (wet). Dry AMD refers to the deterioration of the macula which is the tissue in the center of the retina. Wet AMD refers to the failure of blood vessels under the retina leading to leaking of blood and fluid. Several human and animal studies have identified complement proteins that are related to AMD and novel therapeutic strategies included controlling complement activation pathways, as discussed by Jha et al. in Mol Immunol. 2007; 44(16): 3901-8. In some embodiments, complement inhibitor compounds and compositions of the present disclosure may be used to treat, prevent, or delay development of AMD by inhibiting ocular complement activation. Methods of the present disclosure involving the use of complement inhibitor compounds and compositions for prevention and/or treatment of AMD may include any of those taught in US publication Nos. US2011/0269807 or US2008/0269318, the contents of each of which are herein incorporated by reference in their entirety.

### Corneal Disease

Ocular indications may include corneal disease. The complement system plays an important role in protection of the cornea from pathogenic particles and/or inflammatory antigens. The cornea is the outermost front part of the eye covering and protecting the iris, pupil and anterior chamber and is therefore exposed to external factors. Corneal diseases include, but are not limited to, keratoconus, keratitis, ocular herpes and/or other diseases. Corneal complications may cause pain, blurred vision, tearing, redness, light sensitivity and/or corneal scarring. The complement system is critical for corneal protection, but complement activation may cause damage to the corneal tissue after an infection is cleared as certain complement compounds are heavily expressed. In some embodiments, complement inhibitor compounds and compositions of the present disclosure may be used to treat, prevent, or delay development of corneal diseases by inhibiting ocular complement activation. Methods of the present disclosure for modulating complement activity in the treatment of corneal disease may include any of those taught by Jha et al. in Mol Immunol. 2007; 44(16): 3901-8, the contents of which are herein incorporated by reference in their entirety.

### Autoimmune Uveitis

Ocular indications may include autoimmune uveitis. Uvea is the pigmented area of the eye including the choroids, iris and ciliary body of the eye. Uveitis causes redness, blurred vision, pain, synechia and may eventually cause blindness. Studies have indicated that complement activation products are present in the eyes of patients with autoimmune uveitis and complement plays an important role in disease development. In some embodiments, complement inhibitor compounds and compositions of the present disclosure may be used to treat, prevent, or delay development of uveitis. Such treatments may be carried out according to any of the methods identified in Jha et al. in Mol Immunol. 2007. 44(16): 3901-8, the contents of which are herein incorporated by reference in their entirety.

### Diabetic Retinopathy

Ocular indications may include diabetic retinopathy, which is a disease caused by changes in retinal blood vessels in diabetic patients. Retinopathy may cause blood vessel swelling and fluid leaking and/or growth of abnormal blood vessels. Diabetic retinopathy affects vision and may eventually lead to blindness. Studies have suggested that activation of complement has an important role in the development of diabetic retinopathy. In some embodiments, complement inhibitor compounds and compositions of the present disclosure may be used to treat, prevent, or delay development of diabetic retinopathy. Complement inhibitor compounds and compositions may be used according to methods of diabetic retinopathy treatment described in Jha et al. Mol Immunol. 2007; 44(16): 3901-8, the contents of which are herein incorporated by reference in their entirety.

### Stargardt's Disease

Ocular indications may include Stargardt's disease. Stargardt's disease, also called recessive Stargardt's macular degeneration is an inherited disease of the eye, with an age of onset within the first two decades of life. Complications from Stargardt's disease may include loss of vision (Radu et al., J. Biol. Chem, 2011 286(21) 18593-18601). The disease results from a mutation in the ABCA4 gene. The hallmark of the disease includes accumulation of lipofuscin. Studies have indicated that accumulating lipofuscin activates the complement cascade (Radu et al., J. Biol. Chem, 2011 286(21) 18593-18601). In addition, studies (Tan et al, PNAS 2016; 113(31) 8789-8794) also show that the ABCA4 gene mutation that affects organelle transport and results in lipofuscin accumulation, also results in downregulation of CD59 on the RPE cell surface making them susceptible to damage by complement activation. In some embodiments, complement inhibitor compounds and compositions of the present disclosure may be used to treat, prevent, or delay development of Stargardt's disease, e.g., by inhibiting ocular complement activation.

### Pregnancy-Related Indications

Therapeutic indications addressed with compounds and/or compositions of the present disclosure may include pregnancy-related indications. As used herein, the term “pregnancy-related indication” refers to any therapeutic indication involving child birth and/or pregnancy. Pregnancy-related indications may include complement-related indications. Pregnancy-related indications may include pre-eclampsia and/or HELLP (abbreviation standing for syndrome features of 1) hemolysis, 2) elevated liver enzymes and 3) low platelet count) syndrome. Pre-eclampsia is a disorder of pregnancy with symptoms including elevated blood pressure, swelling, shortness of breath, kidney dysfunction, impaired liver function and/or low blood platelet count. Pre-eclampsia is typically diagnosed by a high urine protein level and high blood pressure. HELLP syndrome is a combination of hemolysis, elevated liver enzymes and low platelet conditions. Hemolysis is a disease involving rupturing of red blood cells leading to the release of hemoglobin from red blood cells. Elevated liver enzymes may indicate a pregnancy-induced liver condition. Low platelet levels lead to reduced clotting capability, causing danger of excessive bleeding. HELLP is associated with a pre-eclampsia and liver disorder. HELLP syndrome typically occurs during the later stages of pregnancy or after childbirth. It is typically diagnosed by blood tests indicating the presence of the three conditions it involves. Typically HELLP is treated by inducing delivery.

Studies suggest that complement activation occurs during HELLP syndrome and pre-eclampsia and that certain complement components are present at increased levels during HELLP and pre-eclampsia. Complement inhibitors of the present disclosure may be used as therapeutic agents to prevent and/or treat these and other pregnancy-related indications. Complement inhibitor compounds and compositions may be used according to methods of preventing and/or treating HELLP and pre-eclampsia taught by Heager et al. in Obstetrics & Gynecology, 1992, 79(1):19-26 or in International publication No. WO2014/078622, the contents of each of which are herein incorporated by reference in their entirety.

### Dosage and Administration

In some embodiments, compounds and/or compositions of the present disclosure may be provided using any dosage and/or route of administration that yields a therapeutically effective result.

In some cases, C5 inhibitors are administered at a milligram dosage. Such doses may include from about 0.01 mg to about 1 mg, from about 0.05 mg to about 2 mg, from about 0.1 mg to about 10 mg, from about 0.5 mg to about 20 mg, from about 1 mg to about 30 mg, from about 5 mg to about 50 mg, from about 10 mg to about 75 mg, from about 50 mg to about 100, from about 100 mg to about 500 mg, from about 200 mg to about 750 mg, from about 500 mg to about 1000 mg, or at least 1000 mg.

In some embodiments, subjects may be administered a therapeutic amount of a C5 inhibitor compound based on the weight of such subjects. In some cases, compounds are administered at a dose of from about 0.001 mg/kg to about 1.0 mg/kg, from about 0.01 mg/kg to about 2.0 mg/kg, from about 0.05 mg/kg to about 5.0 mg/kg, from about 0.03 mg/kg to about 3.0 mg/kg, from about 0.01 mg/kg to about 10 mg/kg, from about 0.1 mg/kg to about 2.0 mg/kg, from about 0.2 mg/kg to about 3.0 mg/kg, from about 0.4 mg/kg to about 4.0 mg/kg, from about 1.0 mg/kg to about 5.0 mg/kg, from about 2.0 mg/kg to about 4.0 mg/kg, from about 1.5 mg/kg to about 7.5 mg/kg, from about 5.0 mg/kg to about 15 mg/kg, from about 7.5 mg/kg to about 12.5 mg/kg, from about 10 mg/kg to about 20 mg/kg, from about 15 mg/kg to about 30 mg/kg, from about 20 mg/kg to about 40 mg/kg, from about 30 mg/kg to about 60 mg/kg, from about 40 mg/kg to about 80 mg/kg, from about 50 mg/kg to about 100 mg/kg, or at least 100 mg/kg. Such ranges may include ranges suitable for administration to human subjects. Dosage levels may be highly dependent on the nature of the condition; drug efficacy; the condition of the patient; the judgment of the practitioner; and the frequency and mode of administration.

In some cases, compounds of the present disclosure are provided at concentrations adjusted to achieve a desired level of the C5 inhibitor in a sample, biological system, or subject (e.g., plasma level in a subject). As used herein, the term “sample” refers to an aliquot or portion taken from a source and/or provided for analysis or processing. In some embodiments, a sample is from a biological source such as a tissue, cell or component part (e.g. a body fluid, including but not limited to blood, mucus, lymphatic fluid, synovial fluid, cerebrospinal fluid, saliva, amniotic fluid, amniotic cord blood, urine, vaginal fluid and semen). In some embodiments, a sample may be or comprise a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, organs. In some embodiments, a sample is or comprises a medium, such as a nutrient broth or gel, which may contain cellular components, such as proteins or nucleic acid molecule. In some embodiments, a “primary” sample is an aliquot of the source. In some embodiments, a primary sample is subjected to one or more processing (e.g., separation, purification, etc.) steps to prepare a sample for analysis or other use. As used herein, the term “subject” refers to any organism to which a compound in accordance with the present disclosure may be administered, e.g., for experimental, diagnostic, prophylactic, and/or therapeutic purposes. Typical subjects include animals (e.g., mammals such as mice, rats, rabbits, porcine subjects, non-human primates, and humans.)

In some cases, desired concentrations of compounds in a sample, biological system, or subject may include concentrations of from about 0.001 μM to about 0.01 μM, from about 0.005 μM to about 0.05 μM, from about 0.02 μM to about 0.2 μM, from about 0.03 μM to about 0.3 μM, from about 0.05 μM to about 0.5 μM, from about 0.01 μM to about 2.0 μM, from about 0.1 μM to about 50 μM, from about 0.1 μM to about 10 μM, from about 0.1 μM to about 5 μM, or from about 0.2 μM to about 20 μM. In some cases, desired concentrations compounds in subject plasma may be from about 0.1 μg/mL to about 1000 μg/mL. In other cases, desired concentrations of compounds in subject plasma may be from about 0.01 μg/mL to about 2 μg/mL, from about 0.02 μg/mL to about 4 μg/mL, from about 0.05 μg/mL to about 5 μg/mL, from about 0.1 μg/mL to about 1.0 μg/mL, from about 0.2 μg/mL to about 2.0 μg/mL, from about 0.5 μg/mL to about 5 μg/mL, from about 1 μg/mL to about 5 μg/mL, from about 2 μg/mL to about 10 μg/mL, from about 3 μg/mL to about 9 μg/mL, from about 5 μg/mL to about 20 μg/mL, from about 10 μg/mL to about 40 μg/mL, from about 30 μg/mL to about 60 μg/mL, from about 40 μg/mL to about 80 μg/mL, from about 50 μg/mL to about 100 μg/mL, from about 75 μg/mL to about 150 μg/mL, or at least 150 μg/mL. In other embodiments, compounds are administered at a dose sufficient to achieve a maximum serum concentration (Cₘₐₓ) of at least 0.1 μg/mL, at least 0.5 μg/mL, at least 1 μg/mL, at least 5 μg/mL, at least 10 μg/mL, at least 50 μg/mL, at least 100 μg/mL, or at least 1000 μg/mL.

In some embodiments, doses sufficient to sustain compound levels of from about 0.1 μg/mL to about 20 μg/mL are provided to reduce hemolysis in a subject by from about 25% to about 99%.

In some embodiments, compounds are administered daily at a dose sufficient to deliver from about 0.1 mg/day to about 60 mg/day per kg weight of a subject. In some cases, the Cₘₐₓ achieved with each dose is from about 0.1 μg/mL to about 1000 μg/mL. In such cases, the area under the curve (AUC) between doses may be from about 200 μg*hr/mL to about 10,000 μg*hr/mL.

According to some methods of the present disclosure, C5 inhibitor compounds and compositions are provided at concentrations needed to achieve a desired effect. In some cases, C5 inhibitor compounds and compositions are provided at an amount necessary to reduce a given reaction or process by half. The concentration needed to achieve such a reduction is referred to herein as the half maximal inhibitory concentration, or “IC₅₀.” Alternatively, C5 inhibitor compounds and compositions may be provided at an amount necessary to increase a given reaction, activity or process by half. The concentration needed for such an increase is referred to herein as the half maximal effective concentration of “EC₅₀.” The C5 inhibitors of the present disclosure may be present in amounts totaling 0.1-95% by weight of the total weight of the composition.

The C5 inhibitor compounds may be administered by any route which results in a therapeutically effective outcome. The administration routes may include, but are not limited to enteral, gastroenteral, epidural, oral, transdermal, peridural, intracerebral, intracerebroventricular, epicutaneous, intradermal, subcutaneous, nasal administration, intravenous, intraarterial, intramuscular, intracardiac, intraosseous infusion (into the bone marrow), intrathecal (into the spinal canal), intraperitoneal, (into the peritoneum), intravesical infusion, intravitreal, (through the eye), intracavernous injection (into a pathologic cavity), intracavitary (into the base of the penis), intravaginal administration, intrauterine, extra-amniotic administration, transdermal, transmucosal, transvaginal, insufflation (snorting), sublingual, sublabial, enema, eye drops (onto the conjunctiva), in ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal, dental intracomal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration which is then covered by a dressing which occludes the area), ophthalmic (to the external eye), oropharyngeal (directly to the mouth and pharynx), parenteral, percutaneous, periarticular, peridural, perineural, periodontal, rectal, respiratory (by inhaling orally or nasally for local or systemic effect), retrobulbar (behind the pons or behind the eyeball), soft tissue, subarachnoid, subconjunctival, submucosal, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis or spinal.

In some embodiments, C5 inhibitor compounds may be formulated to be suitable for oral delivery. The compounds may be administered in any suitable form, either as a liquid solution, or as a solid form, such as a tablet, pill, capsule, or a powder. Small molecule compounds have the advantage of being suitable for oral delivery, whereas biomolecules generally require other methods, e.g., injection delivery. Oral administration of the C5 inhibitor compounds and compositions may, in some cases, provide advantages over other delivery routes. Such treatment may be advantageous in that patients could provide treatment to themselves in their own home, avoiding the need to travel to a provider or medical facility. Oral administration may avoid complications and risks associated with administration that requires needles, such as infections, loss of venous access, local thrombosis, and hematomas. Oral deliverables may be formulated to be slowly releasing, allowing the medication to be effective over an extended period of time.

In some embodiments, the C5 inhibitor compounds and compositions are provided by subcutaneous administration.

In some embodiments, the C5 inhibitor compounds and compositions are provided by intravenous (IV) administration.

In some embodiments, the C5 inhibitor compounds and compositions are provided by ocular delivery routes including, but not limited to, intraocular, ophthalmic, retrobulbar, intravitreal and/or drops on to the conjunctiva. Such methods may include administration of liquid solution eye drops, eye emulsions, suspensions and ointments, ocular injections, or administration by ocular implant release.

In some embodiments, the C5 inhibitor compounds and compositions are provided by topical delivery methods. Such methods may include administration of a topical solution, e.g. a lotion, cream, ointment, emulsion, gel, foam, or a transdermal patch.

In some embodiments, dosage and/or administration are altered to modulate the half-life (tin) of C5 inhibitor compound levels in a subject or in subject fluids (e.g., plasma). In some cases, tin is at least 1 hour, at least 2 hrs, at least 4 hrs, at least 6 hrs, at least 8 hrs, at least 10 hrs, at least 12 hrs, at least 16 hrs, at least 20 hrs, at least 24 hrs, at least 36 hrs, at least 48 hrs, at least 60 hrs, at least 72 hrs, at least 96 hrs, at least 5 days, at least 6 days, at least 7 days, at least 8 days, at least 9 days, at least 10 days, at least 11 days, at least 12 days, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, or at least 16 weeks.

In some embodiments, C5 inhibitor compounds of the present disclosure may exhibit long terminal tin. Extended terminal ti/2 may be due to extensive target binding and/or additional plasma protein binding. In some cases, C5 inhibitor compounds of the present disclosure exhibit tin values greater than 24 hours in both plasma and whole blood. In some cases, compounds do not lose functional activity after incubation in human whole blood at 37° C. for 16 hours.

In some embodiments, dosage and/or administration are altered to modulate the steady state volume of distribution of C5 inhibitor compounds. In some cases, the steady state volume of distribution of compounds is from about 0.1 mL/kg to about 1 mL/kg, from about 0.5 mL/kg to about 5 mL/kg, from about 1 mL/kg to about 10 mL/kg, from about 5 mL/kg to about 20 mL/kg, from about 15 mL/kg to about 30 mL/kg, from about 10 mL/kg to about 200 mL/kg, from about 20 mL/kg to about 60 mL/kg, from about 30 mL/kg to about 70 mL/kg, from about 50 mL/kg to about 200 mL/kg, from about 100 mL/kg to about 500 mL/kg, or at least 500 mL/kg. In some cases, the dosage and/or administration of compounds is adjusted to ensure that the steady state volume of distribution is equal to at least 50% of total blood volume. In some embodiments, compound distribution may be restricted to the plasma compartment.

In some embodiments, C5 inhibitor compounds of the present disclosure exhibit a total clearance rate of from about 0.001 mL/hr/kg to about 0.01 mL/hr/kg, from about 0.005 mL/hr/kg to about 0.05 mL/hr/kg, from about 0.01 mL/hr/kg to about 0.1 mL/hr/kg, from about 0.05 mL/hr/kg to about 0.5 mL/hr/kg, from about 0.1 mL/hr/kg to about 1 mL/hr/kg, from about 0.5 mL/hr/kg to about 5 mL/hr/kg, from about 0.04 mL/hr/kg to about 4 mL/hr/kg, from about 1 mL/hr/kg to about 10 mL/hr/kg, from about 5 mL/hr/kg to about 20 mL/hr/kg, from about 15 mL/hr/kg to about 30 mL/hr/kg, or at least 30 mL/hr/kg.

Time periods for which maximum concentration of C5 inhibitor compounds in subjects (e.g., in subject serum) are maintained (Tₘₐₓ values) may be adjusted by altering dosage and/or administration (e.g., subcutaneous administration). In some cases, C5 inhibitors have Tₘₐₓ values of from about 1 min to about 10 min, from about 5 min to about 20 min, from about 15 min to about 45 min, from about 30 min to about 60 min, from about 45 min to about 90 min, from about 1 hour to about 48 hrs, from about 2 hrs to about 10 hrs, from about 5 hrs to about 20 hrs, from about 10 hrs to about 60 hrs, from about 1 day to about 4 days, from about 2 days to about 10 days, or at least 10 days.

By “lower” or “reduce” in the context of a disease marker or symptom is meant a statistically significant decrease in such level. The decrease may be, for example, at least 10%, at least 200%, at least 30%, at least 40% or more, and is preferably down to a level accepted as within the range of normal for an individual without such disorder.

By “increase” or “raise” in the context of a disease marker or symptom is meant a statistically significant rise in such level. The increase may be, for example, at least 10%, at least 20%, at least 30%, at least 40% or more, and is preferably up to a level accepted as within the range of normal for an individual without such disorder.

As used herein, the phrases “therapeutically effective amount” and “prophylactically effective amount” refer to an amount that provides a therapeutic benefit in the treatment, prevention, or management of pathological processes or an overt symptom of one or more pathological processes. The specific amount that is therapeutically effective may be readily determined by an ordinary medical practitioner, and may vary depending on factors known in the art, such as, for example, the type of pathological processes, patient history and age, the stage of pathological processes, and the administration of other agents that inhibit pathological processes.

Pharmaceutical compositions of the present disclosure may include a pharmacologically effective amount of a C5 inhibitor compound and a pharmaceutically acceptable carrier. As used herein, “pharmacologically effective amount,” “therapeutically effective amount” or simply “effective amount” refers to that amount of a compound effective to produce the intended pharmacological, therapeutic or preventive result. For example, if a given clinical treatment is considered effective when there is at least a 10% alteration (increase or decrease) in a measurable parameter associated with a disease or disorder, a therapeutically effective amount of a drug for the treatment of that disease or disorder is the amount necessary to effect at least a 10% alteration in that parameter. For example, a therapeutically effective amount of a compound may be one that alters binding of a target to its natural binding partner by at least 10%.

The term “pharmaceutically acceptable carrier” refers to a carrier for administration of a therapeutic agent. Such carriers include, but are not limited to, saline, buffered saline, dextrose, water, glycerol, ethanol, and combinations thereof. For drugs administered orally, pharmaceutically acceptable carriers include, but are not limited to pharmaceutically acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract. Agents included in drug formulations are described further herein below.

Efficacy of treatment or amelioration of disease may be assessed, for example by measuring disease progression, disease remission, symptom severity, reduction in pain, quality of life, dose of a medication required to sustain a treatment effect, level of a disease marker or any other measurable parameter appropriate for a given disease being treated or targeted for prevention. It is well within the ability of one skilled in the art to monitor efficacy of treatment or prevention by measuring any one of such parameters, or any combination of parameters. In connection with the administration of a small molecule compound or pharmaceutical composition thereof, “effective against” a disease or disorder indicates that administration in a clinically appropriate manner results in a beneficial effect for at least a fraction of patients, such as an improvement of symptoms, a cure, a reduction in disease load, reduction in tumor mass or cell numbers, extension of life, improvement in quality of life, a reduction in the need for blood transfusions or other effect generally recognized as positive by medical doctors familiar with treating the particular type of disease or disorder.

A treatment or preventive effect is evident when there is a statistically significant improvement in one or more parameters of disease status, or by a failure to worsen or to develop symptoms where they would otherwise be anticipated. As an example, a favorable change of at least 10% in a measurable parameter of disease, and preferably at least 20%, 30%, 40%, 50% or more may be indicative of effective treatment. Efficacy for a given drug or formulation of that drug may also be judged using an experimental animal model for the given disease as known in the art. When using an experimental animal model, efficacy of treatment is evidenced when a statistically significant modulation in a marker or symptom is observed.

C5 inhibitor compounds and additional therapeutic agents may be administered in combination in the same composition, e.g., parenterally, or may be administered as part of separate compositions or by other methods described herein.

In some embodiments, C5 inhibitors of the present disclosure may be modified and/or formulated for various forms of administration. For example, the C5 inhibitors may be modified and/or formulated as active metabolites. As used herein, the term “active metabolite” refers to a form of a compound resulting when a compound is metabolized by the body. The active metabolite of a compound may have the same, reduced, or enhanced therapeutic effect when compared to the unmetabolized form. In some cases, the active metabolite may cause fewer or no side-effects compared with the unmetabolized form.

In some embodiments, C5 inhibitors of the present disclosure may be modified and/or formulated to enhance absorption, distribution, metabolism and/or excretion. In some embodiments, modifications may include preparation as a prodrug. As used herein, the term “prodrug” refers to an inactive compound that may be metabolized to generate an active form. Such active forms may be pharmacologically active. C5 inhibitor prodrugs may vary in one or more of absorption, distribution, metabolism and/or excretion as compared to an unmodified C5 inhibitor. C5 inhibitor prodrugs may have improved bioavailability. C5 inhibitor prodrugs, including, but not limited to, C5 inhibitor prodrugs with improved bioavailability, may have enhanced properties suitable for oral administration when compared to unmodified C5 inhibitors.

In some embodiments, compounds of the present disclosure may be used in in vitro and in vivo ADME (Absorption, Distribution. Metabolism, Excretion) assays to determine their pharmacological properties of absorption, distribution, metabolism, and excretion. In vitro ADME assays include, but are not limited to, plasma protein binding assays, plasma stability assays, hepatocyte stability assays, microsomal binding and stability assays, and permeability assays. In vivo evaluation of ADME characteristics may be carried out by, but not limited to, metabolite profiling, bioavailability and tissue distribution techniques. Characterization of ADME properties of the compounds described in the present disclosure may be used to determine/improve dosage and administration methods.

In some embodiments, C5 inhibitors may include one or more modifications of a phenyl glycinol functional site to form a prodrug. Such modifications may include the addition of an ester group or a phosphate group. As an example, Formulas IIa and IIb present prodrug structures based on compound SM0011 with an ester group and a phosphate group, respectively.

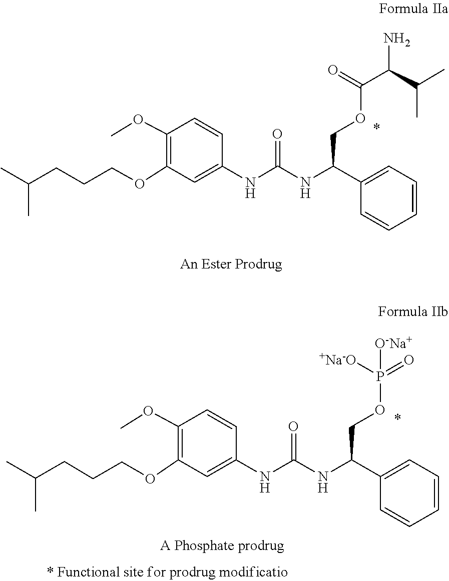

### III. Definitions

The term “aliphatic” or “aliphatic group” as used herein, refers to a straight or branched C₁-C₈ hydrocarbon chain or a monocyclic C₃-C₅ hydrocarbon or bicyclic C₈-C₁₂ hydrocarbon which is fully saturated or that contains one or more units of unsaturation, that is completely saturated or that contains one or more units of unsaturation, but which is not aromatic (also referred to herein as “carbocycle” or “cycloalkyl”), and that has a single point of attachment to the rest of the molecule wherein any individual ring in the bicyclic ring system has 3-7 members. Examples of suitable aliphatic groups include, but are not limited to, linear or branched alkyl, alkenyl, alkynyl groups and hybrids thereof such as (cycloalkyl)alkyl, (cycloalkenyl)alkyl or (cycloalkyl)alkenyl.

The terms “alkyl”, “alkoxy”, “hydroxyalkyl”, “alkoxyalkyl”, and “alkoxycarbonyl”, as used herein, include both straight and branched chains containing one to twelve carbon atoms, and/or which may or may not be substituted.

The terms “alkenyl” and “alkynyl” as used herein alone or as part of a larger moiety shall include both straight and branched chains containing two to twelve carbon atoms.

The term “aromatic” as used herein, refers to an unsaturated hydrocarbon ring structure with delocalized pi electrons. As used herein “aromatic” may refer to monocyclic, bicyclic, or polycyclic aromatic compounds.

The term “aryl” as used herein alone or as part of a larger moiety as in “aralkyl”, “aralkoxy”, or “aryloxyalkyl”, refers to monocyclic, bicyclic and tricyclic carbocyclic ring systems having a total of five to fourteen ring members, wherein at least one ring is aromatic and wherein each ring in the system contains 3 to 8 ring members. The term “aryl” may be used interchangeably with the term “aryl ring.”

The term “bond” as used herein, refers to any chemically feasible bonding configuration that connects immediately adjacent atoms and/or functional groups.

The term “functional group” as used herein, refers to a specific portion of a molecule that is responsible for certain characteristic chemical properties of the molecule. A molecule may have one or more functional groups.

The terms “haloalkyl”, “haloalkenyl” and “haloalkoxy” as used herein refer to alkyl, alkenyl or alkoxy, optionally substituted with one or more halogen atoms. The term “halogen” refers to F, Cl, Br, or I.

The term “heteroatom” as used herein refers to nitrogen, oxygen, or sulfur and includes any oxidized form of nitrogen and sulfur, and the quaternized form of any basic nitrogen.

The term “heterocycle”, “heterocyclyl”, or “heterocyclic” as used herein refers to monocyclic, bicyclic or tricyclic ring systems having three to fourteen ring members in which one or more ring members is a heteroatom, wherein each ring in the system contains 3 to 7 ring members and is non-aromatic.

The term “heteroaryl” as used herein alone or as part of a larger moiety as in “heteroaralkyl” or “heteroalkylalkoxy”, refers to monocyclic, bicyclic and tricyclic ring systems having a total of five to fourteen ring members, and wherein: 1) at least one ring in the system is aromatic; 2) at least one ring in the system contains one or more heteroatoms; and 3) each ring in the system contains 3 to 7 ring members. The term “heteroaryl” may be used interchangeably with the term “heteroaryl ring” or the term “heteroaromatic.”

An aryl (including aralkyl, aralkoxy, aryloxyalkyl and the like) or heteroaryl (including heteroaralkyl, heteroalkylalkoxy and the like) group can contain one or more substituents. Substituents on the unsaturated carbon atom of an aryl, heteroaryl, aralkyl, or heteroaralkyl group can be selected from the group including, but not limited to: halogen; haloalkyl; —CF₃; —R^{∘}; —OR^{∘}; —SR^{∘}; 1,2-methylene-dioxy; 1,2-ethylenedioxy; dimethyleneoxy; protected OH (such as acyloxy); phenyl (Ph); Ph substituted with R^{∘}; —O(Ph); —O—(Ph) substituted with R^{∘}; —CH₂(Ph); —CH₂(Ph) substituted with R^{∘}; —CH₂CH₂(Ph); —CH₂CH₂(Ph) substituted with R^{∘}; —NO₂, —CN, —N(R^{∘})₂, —NR^{∘}C(O)R; —NR^{∘}C(O)N(R^{∘})₂; —NR^{∘}CO₂Rv; —NR^{∘}NR^{∘}C(O)R^{∘}; —NR^{∘}NR^{∘}C(O)N(R^{∘})₂; —NR^{∘}NR^{∘}CO₂R^{∘}; —C(O)C(O)R^{∘}; —C(O)CH₂C(O)R^{∘}; —CO₂R^{∘}; —C(O)R^{∘}; —C(O)N(R^{∘})₂; —OC(O)N(R^{∘})₂; —S(O)₂R; —SO₂N(R^{∘})₂; —S(O)R^{∘}; —NR^{∘}SO₂N(R^{∘})₂, —NR^{∘}SO₂R^{∘}, —C(═S)N(R^{∘})₂, —C(═N H)—N(R^{∘})₂, —(CH₂)_{y}NHC(O)R^{∘}; —(CH₂)_{y}R^{∘}; —(CH₂)_{y}NHC(O)NHR^{∘}; —(CH₂)NHC(O)OR^{∘}; —(CH₂)_{y}NHS(O)R^{∘}; —(CH₂)_{y}NHSO₂R^{∘}; or —(CH₂)_{y}NHC(O)CH(V_{z}—R^{∘})(R^{∘}), wherein each R^{∘} is independently selected from hydrogen, optionally substituted C₁₋₆ aliphatic, an unsubstituted 5-6 membered heteroaryl or heterocyclic ring, phenyl (Ph), —O(Ph), or —CH₂(Ph)-CH₂(Ph), wherein y is 0-6; z is 0-1; and V is a linker group. When R^{∘} is C₁₋₆ aliphatic, it is optionally substituted with one or more substituents selected from —NH₂, —NH(C₁₋₄ aliphatic), —N(C₁₋₄ aliphatic)₂, —S(O) (C₁₋₄ aliphatic- aliphatic), —SO₂(C₁₋₄ aliphatic), halogen, —(C₁₋₄ aliphatic), —OH, —O—(C₁₋₄ aliphatic), —NO₂, —CN, —CO₂H, —CO₂(C₁₋₄ aliphatic), —O(halo C₁₋₄ aliphatic), or -halo(C₁₋₄ aliphatic); wherein each C₁₋₄ aliphatic is unsubstituted.

An aliphatic group or a non-aromatic heterocyclic ring as described above may contain one or more substituents. Substituents on the saturated carbon of an aliphatic group or of a non-aromatic heterocyclic ring are selected from those listed above for the unsaturated carbon of an aryl or heteroaryl group and the following: ═O, ═S, ═NN(R*)₂, ═N—, ═NNHC(O)R*, ═ONNHCO₂(alkyl), ═NNHSO₂(alkyl), or ═NR*, where each R* is independently selected from hydrogen or an optionally substituted C₁₋₄ aliphatic. When R* is C C₁₋₆ aliphatic, it is optionally substituted with one or more substituents selected from —NH₂, —NH(C C₁₋₄ aliphatic), —N(C₁₋₄ aliphatic)₂, halogen, —OH, —O—(C₁₋₄ aliphatic), —NO₂, —CN, —CO₂H, —CO₂(C C₁₋₄ aliphatic), —O(halo C₁₋₄ aliphatic), or -halo(C₁₋₄ aliphatic); wherein each C₁₋₄ aliphatic is unsubstituted.

Substituents on a nitrogen of a non-aromatic heterocyclic ring can be selected from —R⁺, —N(R⁺)₂, —C(O)R⁺, —CO₂R⁺, —C(O)C(O)R⁺, —C(O)CH₂C(O)R⁺, —SO₂R⁺, —SO₂N(R⁺)₂, —C(═S)N(R⁺)₂, —C(═NH)—N(R⁺)₂, or —NR⁺SO₂R⁺; wherein each R* is independently selected from hydrogen, an optionally substituted C₁₋₆ aliphatic, optionally substituted phenyl (Ph), optionally substituted —O(Ph), optionally substituted —CH₂(Ph), optionally substituted —CH₂CH₂(Ph), or an unsubstituted 5-6 membered heteroaryl or heterocyclic ring. When R⁺ is a C₁₋₆ aliphatic group or a phenyl ring, it is optionally substituted with one or more substituents selected from —NH₂, —NH(C₁₋₄ aliphatic), —N(C₁₋₄ aliphatic)₂, halogen, —(C₁₋₄ aliphatic), —OH, —O—(C aliphatic), —NO₂, —CN, —CO₂H, —CO₂(C₁₋₄ aliphatic), —O(halo C₁₋₄ aliphatic), or -halo(C₁₋₄ aliphatic); wherein each C₁₋₆ aliphatic is unsubstituted.

The term “linker group” or “linker” as used herein, refers to an organic moiety that connects two parts of a compound. Linkers are comprised of —O—, —S—, —NR*—, —C(R*)₂—, —C(O)—, or an alkylidene chain. The alkylidene chain is a saturated or unsaturated, straight or branched, C₁₋₆ carbon chain which is optionally substituted, and wherein up to two non-adjacent saturated carbons of the chain are optionally replaced by —C(O)—, —C(O)C(O)—, —C(O)NR*—, —C(O)NR*NR*—, —CO₂—, —OC(O)—, —NR*CO₂—, —O—, —NR*C(O)NR*—, —OC(O)NR*—, —NR*NR*—, —NR*C(O)—, —S—, —SO—, —SO₂—, —NR*—, —SO₂NR*—, or —NR*SO₂—; wherein R* is selected from hydrogen or C₁₋₄ aliphatic. Optional substituents on the alkylidene chain are as described above for an aliphatic group.

The term “nitrogen” may be N, NH or NR⁺. For example, in a ring having 0-3 heteroatoms selected from oxygen, sulfur or nitrogen, the nitrogen may be N (as in 3,4-dihydro-2H-pyrrolyl), NH (as in pyrrolidinyl) or NR⁺ (as in N-substituted pyrrolidinyl).

The term “saturated,” as used herein, refers to a hydrocarbon which has single bonds between the carbon atoms and may contain other atoms.

The term “substitution” as used herein, refers to the replacement of a functional group of a chemical compound by another functional group.

The term “substituent” as used herein, refers to an atom or functional group other than hydrogen, that replaces a hydrogen atom on a hydrocarbon. Non-limiting examples of substituents include atoms (e.g., halogens, heteroatoms) and functional groups (e.g. aliphatic groups, aryl groups, and heteroaryl groups, —OH, oxo, —O—(C₁-C₄)-alkyl, halogen, —CF₃, nitrile, —COOH, —CO—NH₂, —O—CO—NH₂, —OCF₃, —N(H) (C₁-C₄-alkyl), and —N—(C₁-C₄-alkyl)₂).

The term “unsaturated” as used herein, refers to a hydrocarbon which has double or triple bonds between the carbon atoms and may contain other atoms. The term “partially unsaturated” as used herein, refers to hydrocarbon that has at least one double or triple bond between the carbon atoms and may contain other atoms.

### IV. Equivalents and Scope

Those skilled in the art will recognize, or be able to ascertain using no more than routine experimentation, many equivalents to the specific embodiments in accordance with the invention described herein. The scope of the present invention is not intended to be limited to the above description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the invention in its broader aspects.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention.

### EXAMPLES

### Example 1. Compound Analysis by Liquid Chromatography-Mass Spectrometry (LC-MS)

Compounds, indicated in Table 1, were synthesized according to the methods described below or according to standard methods known in the art [see, e.g. Morrison and Boyd in “Organic Chemistry”, 6^{th} edition, Prentice Hall (1992)] and validated for proper structure by mass spectrometry. Compounds were analyzed by Liquid chromatography-mass spectrometry (LCMS) after synthesis to confirm mass-to-charge ratio (m/z [M+H]).

Analytical LCMS was performed by Waters Acquity SDS using a linear gradient of 5% to 100% B over a 5 minute gradient, and UV visualization with a diode array detector. The column used was a C18 Acquity UPLC BEH, 2.1 mm i.d. by 50 mm length, 1.7 μM with flow rate of 0.6 ml/min. Mobile phase A was water and mobile phase B was acetonitrile (0.1% TFA). Results are shown in Table 4.

TABLE 4
LCMS assay data

| | m/z found |
|---|---|
| ID# | [M + H] |
| SM0002 | 417.4 |
| SM0003 | 417.6 |
| SM0004 | 373.4 |
| SM0005 | 371.7 |
| SM0006 | 387.5 |
| SM0007 | 385.5 |
| SM0009 | 371.5 |
| SM0010 | 385.4 |
| SM0011 | 387.4 |
| SM0012 | 387.4 |
| SM0013 | 397.4 |
| SM0014 | 381.4 |
| SM0015 | 389.4 |
| SM0016 | 373.4 |
| SM0018 | 405.6 |
| SM0019 | 399.5 |
| SM0020 | 405.5 |
| SM0021 | 371.4 |
| SM0022 | 396.3 |
| SM0023 | 405.4 |
| SM0024 | 405.6 |
| SM0025 | 427.5 |
| SM0026 | 357.4 |
| SM0027 | 397.6 |
| SM0028 | 417.5 |
| SM0029 | 403.8 |
| SM0030 | 403.4 |
| SM0031 | 358.5 |
| SM0032 | 377.4 |
| SM0033 | 415.6 |
| SM0034 | 357.5 |
| SM0036 | 401.5 |
| SM0037 | 372.5 |
| SM0038 | 401.5 |
| SM0039 | 363.7 |
| SM0040 | 388.5 |
| SM0041 | 387.4 |
| SM0043 | 414.4 |
| SM0044 | 408.4 |
| SM0045 | 358.4 |
| SM0046 | 352.3 |
| SM0047 | 387.5 |
| SM0048 | 436.4 |
| SM0049 | 358.4 |
| SM0050 | 387.4 |
| SM0051 | 380.3 |
| SM0052 | 363.4 |
| SM0053 | 401.5 |
| SM0054 | 421.4 |
| SM0055 | 391.5 |
| SM0056 | 385.4 |
| SM0058 | 375.4 |
| SM0059 | 364.4 |
| SM0061 | 369.2 |
| SM0062 | 429.5 |
| SM0063 | 414.5 |
| SM0064 | 401.5 |
| SM0066 | 399.4 |
| SM0067 | 382.4 |
| SM0068 | 431.0 |
| SM0069 | 359.4 |
| SM0070 | 357.4 |
| SM0072 | 373.4 |
| SM0075 | 375.4 |
| SM0076 | 382.4 |
| SM0078 | 388.4 |
| SM0079 | 435.8 |
| SM0080 | 359.4 |
| SM0081 | 387.4 |
| SM0082 | 419.5 |
| SM0083 | 389.5 |
| SM0085 | 385.1 |
| SM0086 | 408.4 |
| SM0087 | 327.4 |
| SM0089 | 435.4 |
| SM0090 | 359.4 |
| SM0091 | 367.5 |
| SM0092 | 401.5 |
| SM0093 | 414.6 |
| SM0096 | 353.2 |
| SM0099 | 337.0 |
| SM0100 | 414.5 |
| SM0101 | 371.4 |
| SM0102 | 400.5 |
| SM0103 | 399.4 |
| SM0104 | 423.5 |
| SM0105 | 374.4 |
| SM0106 | 435.5 |
| SM0107 | 386.4 |
| SM0108 | 449.3 |
| SM0109 | 325.4 |
| SM0111 | 399.5 |
| SM0112 | 357.4 |
| SM0113 | 435.4 |
| SM0116 | 370.4 |
| SM0117 | 371.4 |
| SM0118 | 401.5 |
| SM0119 | 370.4 |
| SM0120 | 401.5 |
| SM0121 | 414.5 |
| SM0200 | 500.5 |
| SM0201 | 498.5 |
| SM0202 | 557.5 |
| SM0203 | 522.5 |
| SM0204 | 449.2 |
| SM0205 | 539.2 |
| SM0206 | 546.2 |
| SM0207 | 542.2 |
| SM0208 | 559.2 |
| SM0209 | 573.3 |
| SM0210 | 518.2 |
| SM0211 | 476.2 |
| SM0212 | 462.1 |
| SM0213 | 448.1 |
| SM0214 | 463.1 |
| SM0215 | 553.2 |
| SM0216 | 490.2 |
| SM0217 | 529.2 |
| SM0218 | 473.5 |
| SM0219 | 451.0 |

### Example 2. Compound Analysis by Surface Plasmon Resonance (SPR)

Surface plasmon resonance (SPR) technology was used to evaluate affinity, specificity, and kinetics of interactions between compounds and complement protein C5 in real time without the need for labeling.

SensiQ FE SPR system (SensiQ Technologies, Oklahoma City, Okla.) was used to provide sensitive and accurate detection of binding of small molecules to the very large C5 protein (MW=195,000 Da). The chip was prepared by preconditioning the sensor according to the protocol of the SensiQ FE using 10 mM HCl, 50 mM NAOH and 0.1% SDS. The sensor chip was activated by using a mixture of fresh EDAC (1-ethyl-3-(-3-dimethylaminopropyl) carbodiimide) (Sigma Co., St. Louis, Mo.) and NHS (N-hydroxy succinimide) (Sigma Co., St. Louis, Mo.). Human C5 was surface immobilized to a Pioneer Biosensor chip via random amine coupling (>12,000 RU) which makes use of the N-terminus and ε-amino groups of lysine residues of the protein ligand. Immobilization was done by injecting 30-40 μg/ml C5 in 10 mM NaAc pH 4.5 onto designated channels at a rate of 10 μL/minute for 12 minutes, targeting RL>12000 RU for small molecules.

Compounds were diluted in DMSO in a format of 100-fold final concentration and 3-fold serial dilution (5 or 6 dilutions). The 100-fold compounds were transferred to one-fold DMSO-free assay buffer in the 96-well test plate. The compound solution was injected at a rate of 60 μL/minute for 30-60 seconds, followed by 60-90 seconds dissociation time, buffer flushing and/or priming. Blank solution (1% DMSO assay buffer) was run for every 6 injections of compounds. Double reference by subtracting both blank channel and reference channel was applied for data processing. Titration of C5 binding compounds to the C5-immobilized biosensor chip surface led to interactions between C5 and potential binders, and the resulting changes of surface refractive index were sensitively measured by the system.

SPR data were analyzed with the managing software provided by SensiQ and equilibrium dissociation constant (K_{D}) values were determined for each compound. Compounds with K_{D} values less than 10 μM are presented in Table 5. Where a range of compound concentrations were analyzed, the lowest value obtained is presented.

TABLE 5
SPR results

| | K_{D} |
|---|---|
| ID# | (nM) |
| SM0002 | 14 |
| SM0003 | 14 |
| SM0013 | 17 |
| SM0006 | 18 |
| SM0032 | 25 |
| SM0004 | 28 |
| SM0005 | 37 |
| SM0019 | 39 |
| SM0008 | 45 |
| SM0012 | 47 |
| SM0001 | 49 |
| SM0014 | 63 |
| SM0044 | 74 |
| SM0022 | 84 |
| SM0007 | 90 |
| SM0023 | 92 |
| SM0026 | 97 |
| SM0050 | 102 |
| SM0010 | 107 |
| SM0040 | 110 |
| SM0017 | 111 |
| SM0059 | 113 |
| SM0018 | 114 |
| SM0100 | 120 |
| SM0024 | 130 |
| SM0069 | 140 |
| SM0080 | 144 |
| SM0101 | 150 |
| SM0011 | 157 |
| SM0027 | 171 |
| SM0067 | 172 |
| SM0030 | 190 |
| SM0015 | 196 |
| SM0039 | 210 |
| SM0104 | 210 |
| SM0029 | 239 |
| SM0093 | 240 |
| SM0016 | 290 |
| SM0066 | 290 |
| SM0084 | 310 |
| SM0054 | 320 |
| SM0021 | 368 |
| SM0094 | 380 |
| SM0033 | 410 |
| SM0058 | 500 |
| SM0092 | 540 |
| SM0052 | 564 |
| SM0113 | 601 |
| SM0025 | 620 |
| SM0037 | 628 |
| SM0009 | 647 |
| SM0116 | 690 |
| SM0038 | 800 |
| SM0047 | 830 |
| SM0119 | 830 |
| SM0091 | 990 |
| SM0075 | 1300 |
| SM0034 | 1400 |
| SM0065 | 1600 |
| SM0081 | 1600 |
| SM0095 | 1600 |
| SM0102 | 1700 |
| SM0103 | 1800 |
| SM0043 | 1930 |
| SM0062 | 2000 |
| SM0071 | 2100 |
| SM0090 | 2100 |
| SM0096 | 2100 |
| SM0097 | 2200 |
| SM0068 | 2600 |
| SM0105 | 2600 |
| SM0031 | 2700 |
| SM0106 | 2700 |
| SM0045 | 2800 |
| SM0089 | 3100 |
| SM0073 | 3200 |
| SM0107 | 3200 |
| SM0108 | 3200 |
| SM0053 | 3348 |
| SM0074 | 3600 |
| SM0078 | 3600 |
| SM0098 | 3800 |
| SM0109 | 3800 |
| SM0061 | 3900 |
| SM0110 | 3900 |
| SM0049 | 4100 |
| SM0036 | 4300 |
| SM0111 | 4700 |
| SM0070 | 4800 |
| SM0077 | 5200 |
| SM0057 | 5300 |
| SM0112 | 5500 |
| SM0099 | 5700 |
| SM0063 | 6368 |
| SM0042 | 6400 |
| SM0083 | 6400 |
| SM0114 | 6400 |
| SM0064 | 6610 |
| SM0115 | 6700 |
| SM0048 | 7200 |
| SM0117 | 7600 |
| SM0118 | 8100 |
| SM0072 | 8200 |
| SM0046 | 8600 |
| SM0076 | 9400 |
| SM0120 | 9500 |
| SM0121 | 9500 |
| SM0087 | 9900 |

### Example 3. Hemolysis Assay (Optical)

Experiments were carried out using a red blood cell (RBC) hemolysis assay to assess compound inhibition of RBC lysis. This assay identifies compounds capable of reducing sheep erythrocyte lysis via terminal complex formation. The assay was carried out using 1.5% human C5 depleted sera and 0.5 nM purified human C5.

GVB++ buffer (Complement Technology, Tyler, Tex.) was heated at 37° C. for a minimum of 20 minutes. The human C5 depleted sera and purified human C5 were rapidly thawed at 37° C. and then stored on ice or wet ice, respectively. The compound stock (10 mM, DMSO) was serially diluted in 100% DMSO to obtain ten 6-fold dilutions before addition of GVB++. Sera dilution was prepared by adding 5 mL of GVB++ to a 15 mL conical tube, removing 600 μL of the GVB++ and adding 600 μL of the 100% sera. The tube was mixed by inverting three times. A volume of 25 μL of the diluted sera was added to each well so that the final concentration of sera in the well was 1.5%. C5 dilution was prepared by adding 5 mL of GVB++ to a 15 mL conical tube, removing 4 μL of the GVB++ and adding 4 μL of the C5 stock. The tube was mixed by inverting three times. A volume of 25 μL was added to each well so that the final amount of C5 was 0.5 nM in each well. The antibody-sensitized sheep erythrocytes (EAs) were centrifuged at 1,000× gravity at 22° C. for 3 minutes. The supernatant was pipetted off without disrupting the pellet. The pellet was then resuspended in GVB++ with the same volume removed. Resuspended EAs were mixed by gently inverting the tube.

Five controls were run on each plate: (1) EAs only=100 μL EAs+50 μL GVB++ with 4% DMSO+50 μL GVB++; (2) EA+Sera=100 μL EAs+50 μL GVB++ with 4% DMSO+25 μL Sera dilution+25 μL GVB++; (3) EA+C5=100 μL EAs+50 μL GVB++ with 4% DMSO+25 μL C5 dilution+25 μL GVB++; (4) EA+Sera+C5=100 μL EAs+50 μL GVB++ with 4% DMSO+25 μL Sera dilution+25 μL C5; (5) GVB++ Only=200 μL GVB++. Other wells included: GVB++ with 4% DMSO=20 μL DMSO+480 μL GVB++. All samples were analyzed in duplicate. The compound dose response curve was generated using samples prepared with 100 μL EA+50 μL compound dilution+25 μL C5 dilution+25 μL sera dilution.

Test plates were prepared by adding 100 μL of EAs, 50 μL of compound dilution, 25 μL of sera dilution, and 25 μL of C5 dilution to individual wells of a 96-well tissue culture-treated clear microtitre plate (USA Scientific, Ocala, Fla.) and resuspending by pipetting up and down three times. The samples were incubated at 37° C. for one hour. At the completion of the incubation, the plates were centrifuged at 1,000× gravity for 3 minutes. 100 μl of supernatant were transferred to a new plate and the absorbance was read at 412 nm. Data was fit with a log-logit formula producing a dose-response curve and IC₅₀.

Compounds with IC₅₀ values less than 20 μM are presented in Table 6. “IC₅₀” refers to the half maximal inhibitory concentration, where the value is the concentration of the inhibitor needed to reduce red blood cell hemolysis by half. Where replicate analysis was conducted, average IC₅₀ values are provided along with standard deviation (SD) values.

TABLE 6
Hemolysis inhibitory activity

| | IC₅₀ | SD |
|---|---|---|
| ID# | (nM) | (nM) |
| SM0001 | 20.9 | 10.0 |
| SM0002 | 21.9 | 7.5 |
| SM0003 | 26.8 | 9.7 |
| SM0004 | 42.6 | 19.9 |
| SM0005 | 65.8 | 16.5 |
| SM0006 | 125.0 | 25.5 |
| SM0007 | 165.3 | 77.6 |
| SM0008 | 175.7 | 22.1 |
| SM0009 | 198.3 | |
| SM0010 | 226.4 | 36.3 |
| SM0011 | 266.2 | 147.8 |
| SM0012 | 315.9 | |
| SM0013 | 326.6 | 207.8 |
| SM0014 | 334.8 | 120.3 |
| SM0015 | 342.0 | |
| SM0016 | 378.0 | |
| SM0017 | 401.0 | |
| SM0018 | 420.0 | |
| SM0019 | 450.4 | 223.1 |
| SM0020 | 478.0 | |
| SM0021 | 533.0 | |
| SM0022 | 581.0 | 49.5 |
| SM0023 | 607.7 | |
| SM0024 | 614.4 | 580.0 |
| SM0025 | 675.4 | 229.7 |
| SM0026 | 685.2 | |
| SM0027 | 696.5 | 181.9 |
| SM0028 | 896.0 | |
| SM0029 | 900.8 | 182.7 |
| SM0030 | 936.0 | 50.9 |
| SM0031 | 982.8 | 214.7 |
| SM0032 | 1039.3 | 179.3 |
| SM0033 | 1098.0 | |
| SM0034 | 1104.0 | |
| SM0035 | 1179.0 | |
| SM0036 | 1236.0 | |
| SM0037 | 1317.0 | 275.8 |
| SM0038 | 1481.0 | |
| SM0039 | 1489.0 | |
| SM0040 | 1621.0 | |
| SM0041 | 1798.0 | 268.7 |
| SM0042 | 2132.0 | |
| SM0043 | 2140.0 | 306.9 |
| SM0044 | 2180.0 | 362.0 |
| SM0045 | 2232.7 | 536.0 |
| SM0046 | 2245.0 | |
| SM0047 | 2269.0 | |
| SM0048 | 2545.0 | |
| SM0049 | 2790.0 | 288.5 |
| SM0050 | 2865.8 | 1174.8 |
| SM0051 | 3063.0 | |
| SM0052 | 3096.5 | 507.0 |
| SM0053 | 3378.0 | |
| SM0054 | 3394.0 | |
| SM0055 | 3452.0 | |
| SM0056 | 3945.0 | |
| SM0057 | 4049.0 | |
| SM0058 | 4088.5 | 621.5 |
| SM0059 | 4390.0 | 427.1 |
| SM0060 | 4467.0 | |
| SM0061 | 4985.0 | |
| SM0062 | 5652.0 | |
| SM0063 | 6135.5 | 129.4 |
| SM0064 | 6449.0 | |
| SM0065 | 6670.5 | 1166.0 |
| SM0066 | 6873.0 | |
| SM0067 | 7363.5 | 1566.2 |
| SM0068 | 7364.0 | |
| SM0069 | 7720.5 | 71.4 |
| SM0070 | 8603.0 | |
| SM0071 | 8655.0 | |
| SM0072 | 8683.0 | |
| SM0073 | 9255.0 | |
| SM0074 | 9364.0 | |
| SM0075 | 11490.0 | |
| SM0076 | 11680.0 | 820.2 |
| SM0077 | 11820.0 | |
| SM0078 | 12620.0 | |
| SM0079 | 12650.0 | |
| SM0080 | 13005.0 | 374.8 |
| SM0081 | 13040.0 | |
| SM0082 | 14550.0 | |
| SM0083 | 15450.0 | |
| SM0084 | 16080.0 | |
| SM0085 | 16120.0 | |
| SM0086 | 16560.0 | 4242.6 |
| SM0087 | 16700.0 | |
| SM0088 | 18430.0 | |

### Example 4. Hemolysis Assay (Luminescent)

Sheep red blood cells coated with rabbit anti-sheep erythrocyte antiserum (EA cells; Complement Technology, Tyler, Tex.) were used to assay compound inhibitory activity of the classical complement activation pathway. Briefly, the EA cells were washed once and resuspended in the same volume of GVB++ buffer (Complement Technology, Tyler, Tex.). 25 μL of EA cells were then distributed into each well of 384-well tissue culture plates using Apricot iPipette Pro (Apricot Designs; Covina, Calif.). Compounds were tested in 10 points of final concentrations ranging from 16.67 μM to 1.65 μM in a 6-fold titration series. Compounds were dispensed into 384-well plates from 6.7 mM and 3.35 μM DMSO working stocks using an HP Digital Dispenser (HP; Corvallis, Oreg.). The reactions also contained 1.5% (v/v) C5-depleted human serum (Complement Technology). Hemolysis was induced by addition of human C5 (Complement Technology) at a concentration of 0.5 nM and plates were incubated for 1 hour at 37° C. in a cell culture incubator. The extent of hemolysis was measured by ability of released hemoglobin to catalyze luminol in the presence of hydrogen peroxide. Luminescence was then measured using a plate reader.

Luminescence measurements were used to prepare a dose-response curve. From the curve, the half maximal inhibitory concentration (IC₅₀) for each compound was determined, where the IC₅₀ represents the concentration of each compound needed to reduce red blood cell hemolysis by half. Results are presented in Table 7. Numbers in parenthesis following the compound number indicate alternate enantiomers (as distinguished by retention time during chromatographical separation).

TABLE 7
Hemolysis inhibitory activity

| | IC₅₀ |
|---|---|
| ID# | (nM) |
| SM0002 | 11.9 |
| SM0006 | 37.6 |
| SM0218 | 48.6 |
| SM0011 | 60.2 |
| SM0201 | 89.5 |
| SM0200 | 136.8 |
| SM0214 | 175.0 |
| SM0216 | 198.6 |
| SM0217 | 210.0 |
| SM0219 | 239.3 |
| SM0202 | 240.1 |
| SM0215 | 243.7 |
| SM0203 | 255.0 |
| SM0035 | 328.2 |
| SM0104 | 558.1 |
| SM0204 | 752.5 |
| SM0207 | 864.8 |
| SM0212 | 867.4 |
| SM0213 | 902.8 |
| SM0208 | 913.9 |
| SM0209 | 1139.7 |
| SM0205 | 1294.2 |
| SM0211 | 1907.5 |
| SM0206 | 2064.3 |
| SM0210 | 2122.1 |

### Example 5. Drug-Metabolism-and-Pharmacokinetics (DMPK)

Single intravenous (IV) and oral dose (P0, per oral) administration of C5 inhibitors were carried out in rats. Rats were then analyzed for Drug-Metabolism-and-Pharmacokinetic (DMPK) properties, used to determine the pharmacokinetics and oral bioavailability of the C5 inhibitors.

SM0011 was formulated as a clear solution at 1 mg/mL in 5% DMSO: 20% HP-Beta-CD. Fasted male Sprague Dawley rats were dosed with the solution at 1 MPK (mg/kg) IV and 10 MPK (mg/kg) PO. Analysis of the DMPK properties of the compound were used to determine the bioavailability. Results indicated that the bioavailability of SM0011 was about 30%.

### Example 6. Hemolysis Inhibition with Paroxysmal Nocturnal Hemoglobinuria Patient Cells

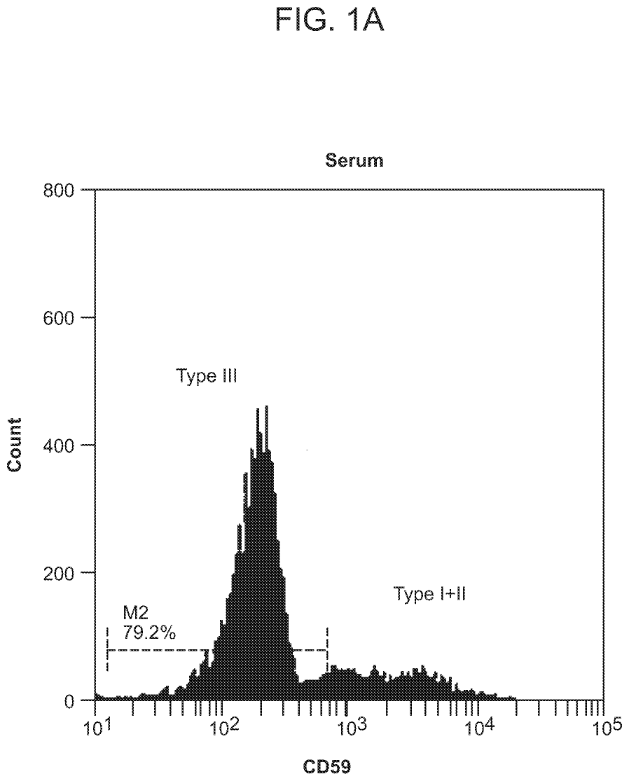

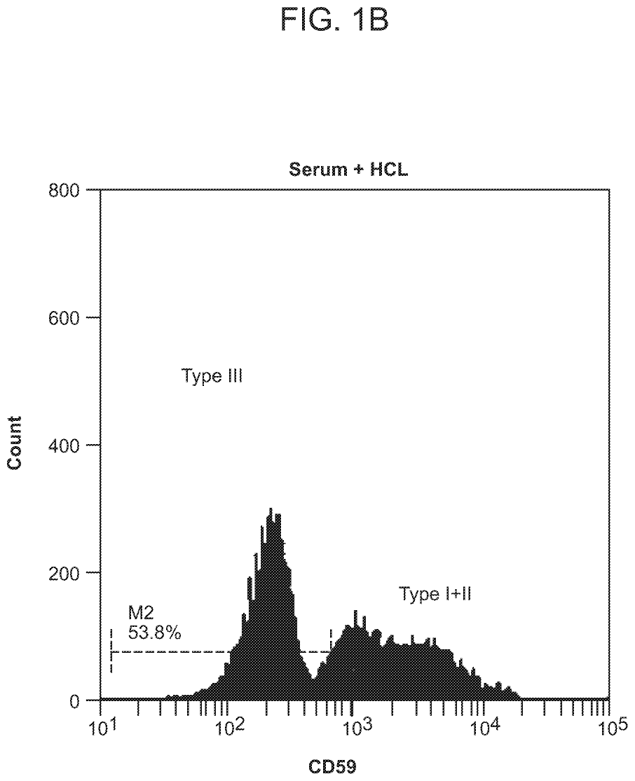

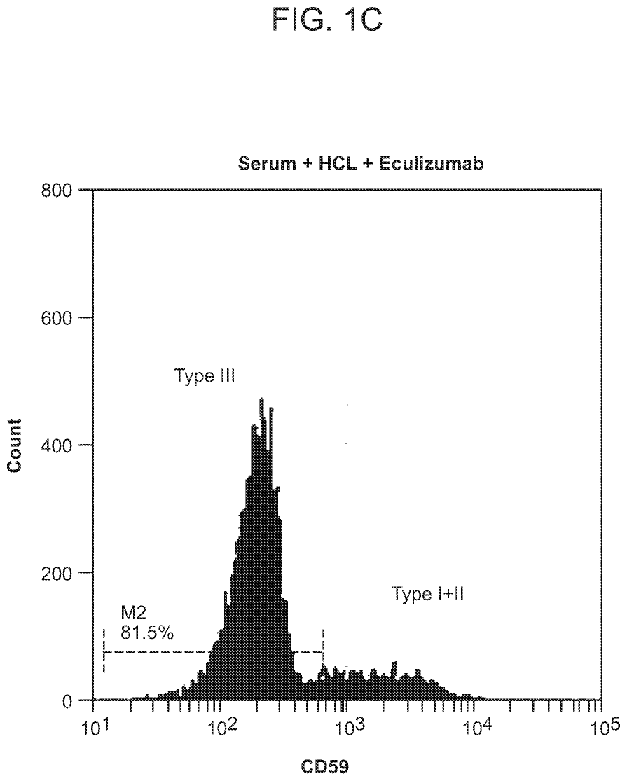

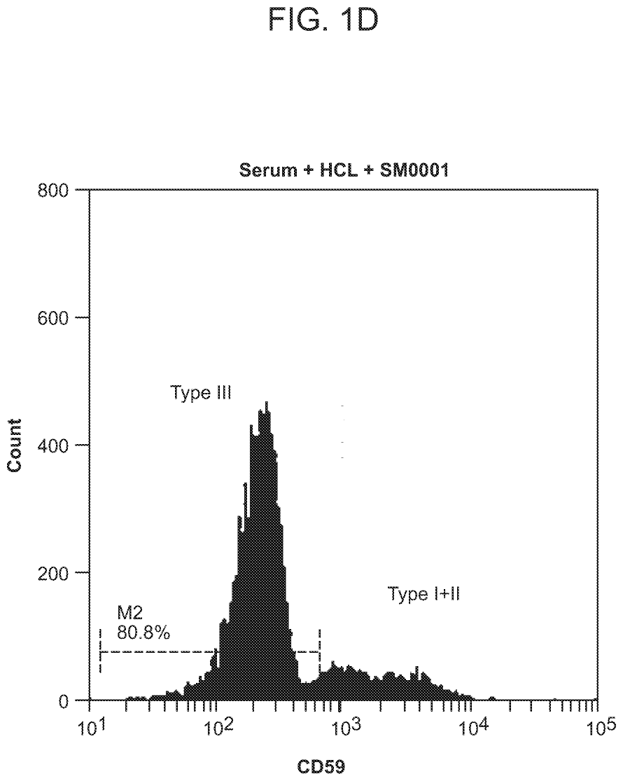

Flow cytometry studies were carried out to assess the ability of compounds to inhibit hemolysis of CD59-deficient RBCs from patients with paroxysmal nocturnal hemoglobinuria (PNH). RBCs collected from PNH patients were washed three times with Alsever's solution, followed by pelleting and re-suspending in GVB++ buffer (Complement Technology, Tyler, Tex.) in a ratio of 1:2. To induce hemolysis, donor-matched serum was acidified to pH 6.4 with HCl. Compounds, serum, and RBCs, 2.5% volume per volume (v/v), were incubated for 18 hours at 37° C. After incubation, cells were washed and re-suspended in 1 ml fluorescence-associated cell sorting (FACS) buffer (0.1% BSA IgG-free in PBS, 0.1% Sodium Azide). Then, anti-CD59 antibody conjugated with phycoerythrin was added at a final concentration of 0.25 μg/ml to 100 μl of cell suspension and incubated at 4° C. for 30 minutes. Cells were then washed twice with cold FACS buffer, re-suspended in FACS buffer and analyzed with a BD Accuri C6 Flow Cytometer (BD Biosciences, San Jose, Calif.) for CD59 levels. The level of CD59-positive cells was monitored as a measure of complement-mediated hemolysis of PNH type III cells. A negative control using non-acidified serum was used to establish a baseline of CD59 expression under non-hemolytic conditions (FIG. 1A). When acidified serum was introduced, the level of CD59 expression decreased, consistent with RBC hemolysis (FIG. 1B). Hemolysis was blocked in the presence of eculizumab, which is a known antibody-based C5 inhibitor (FIG. 1C). Similar inhibition was also observed with SM0001 (FIG. 1D), demonstrating the ability of this compound to inhibit hemolysis in human PNH patient cells and the potential for using this compound as a substitute for eculizumab.

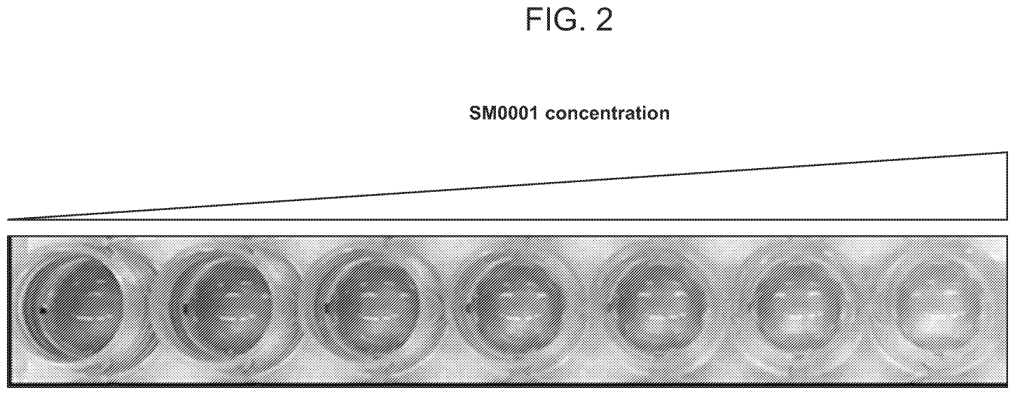

Similar experiments were conducted using increasing concentrations of SM0001. Results, shown in FIG. 2, demonstrate the ability of SM0001 to inhibit PNH patient hemolysis in a dose-dependent manner. In FIG. 2, SM0001 concentration increases from left to right. Wells on the left side have higher levels of hemolysis indicated by a darker color whereas hemolysis decreases with increasing concentration of SM0001.

### Example 7. Synthesis of Intermediates

### Intermediate A1: (R)-2-amino-2-phenylethyl acetate

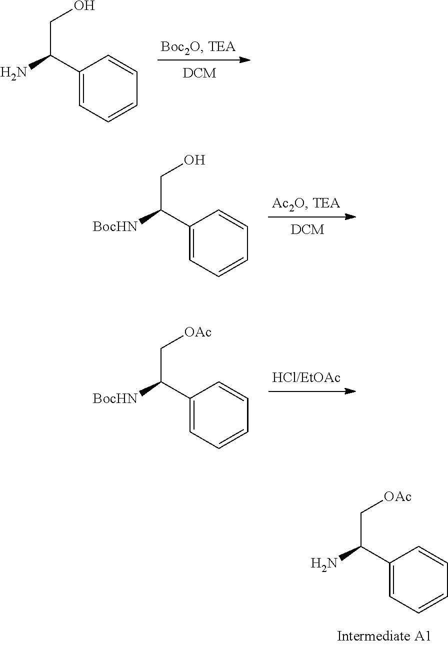 [SMILES: N[C@@H](CO)c1ccccc1 ; CC(C)(C)OC(=O)N[C@@H](CO)c1ccccc1 ; CC(=O)OC[C@H](NC(=O)OC(C)(C)C)c1ccccc1 ; CC(=O)OC[C@H](N)c1ccccc1]

Step 1: Tert-butoxycarbonyl tert-butyl carbonate (79.6 g, 364 mmol, 84 mL, 1.00 eq) is added to a cooled (0° C.) solution of (2R)-2-amino-2-phenyl-ethanol (50.0 g, 364 mmol, 1.00 eq) and triethylamine (44.3 g, 437 mmol, 61 mL, 1.20 eq) in dry tetrahydrofuran (1.40 L). The mixture is stirred at 0° C. for 3 hours then concentrated in vacuo. The residue is dissolved in ethyl acetate (3000 mL) and washed with water, dried over anhydrous Na₂SO₄, filtered and concentrated in vacuo. The residue is purified by silica column chromatography to give tert-butyl N-[(1R)-2-hydroxy-1-phenyl-ethyl]carbamate (64.0 g, 270 mmol, 74% yield) as a yellow solid.

Step 2: A solution of tert-butyl N-[(1R)-2-hydroxy-1-phenyl-ethyl]carbamate (63.0 g, 265 mmol, 1.00 eq) and triethylamine (53.7 g, 531 mmol, 73 mL, 2.00 eq) in dry dichloromethane (1.40 L) is stirred at 25° C. for 1 h. Then acetic anhydride Ac₂O (67.8 g, 664 mmol, 62 mL, 2.50 eq) is added. The mixture is stirred at 25° C. for 2 h. The mixture is diluted with saturated ammonium chloride (200 mL), extracted with dichloromethane (200 mL×3). The combined organic layer is washed with brine, dried over anhydrous sodium sulfate, filtered and concentrated. The residue is purified by column chromatography to afford [(2R)-2-(tert-butoxycarbonylamino)-2-phenyl-ethyl] acetate (63.0 g, 226 mmol, 85% yield) as a white solid.

Step 3: To a solution of [(2R)-2-(tert-butoxycarbonylamino)-2-phenyl-ethyl]acetate (63.0 g, 226 mmol, 1.00 eq) in dry dichloromethane (500 mL) is added HCl/dioxane (4 M, 564 mL). The mixture is stirred at 25° C. for 2 hours. The reaction mixture is concentrated in vacuo. [(2R)-2-amino-2-phenyl-ethyl] acetate (40.0 g, 223 mmol, 99% yield) is obtained as white solid.

### Intermediate A2: 4-Methoxy-3-((4-methylpentyl)oxy)benzoic acid

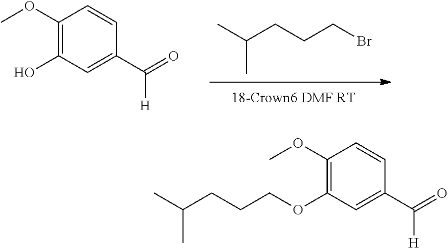

Alkylation of Phenol: To a mixture of 3-hydroxy-4-methoxybenzaldehyde (54.1 mmol, 8.2 g), potassium carbonate (81.2 mmol, 11.2 g), and 18-Crown-6 (1.4 g) in tetrahydrofuran (50 mL) is added 1-bromo-4-methylpentane (54.1 mmol, 7.6 mL). The resulting mixture is stirred for 12 hours then quenched with water and extracted with ethyl acetate. The organics are washed with brine, dried over sodium sulfate, and filtered. The filtrate is concentrated under vacuum to give a crude oil. Purification of the oil is achieved by chromatography on silica using 40% ethyl acetate in n-hexane as eluent to give benzyl 4-Methoxy-3-(((4-methylpentyl)oxy)benzaldehyde (13 g) as a pale oil.

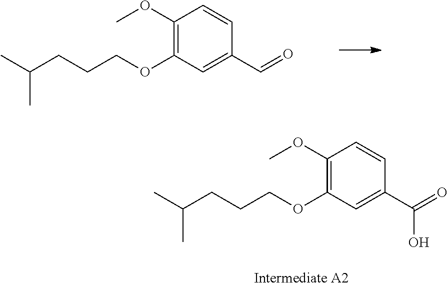 [SMILES: COc1ccc(C=O)cc1OCCCC(C)C ; COc1ccc(C(=O)O)cc1OCCCC(C)C]

Oxidation of isovanilins: To a solution of 4-Methoxy-3-(((4-methylpentyl)oxy)benzaldehyde (54.2 mmol, 12.7 g) in acetonitrile (250 mL) at 0° C. is added 30% hydrogen peroxide solution (81.2 mmol, 2.5 mL), a solution of sodium phosphate monobasic hydrate (0.3 g) and sodium chlorite (75.8 mmol, 6.8 g) in water (20 mL). The resulting solution is warmed to room temperature and allowed to stir for an additional 24 hours. The reaction is quenched with a saturated solution of sodium thiosulfate then extracted with ethyl acetate. The organics are washed with brine then dried over sodium sulfate, and filtered. The filtrate is concentrated under vacuum to give a crude oil. Purification of the oil is achieved by chromatography on silica using 40% ethyl acetate in n-hexane as eluent to give 4-Methoxy-3-(((4-methylpentyl)oxy)benzoic acid (3.1 g) as a white solid.

### Intermediate A3: 3-isohexyloxy-4-methoxy-aniline

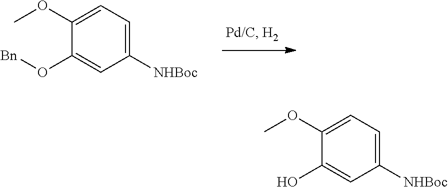 [SMILES: COc1ccc(NC(=O)OC(C)(C)C)cc1OCc1ccccc1 ; COc1ccc(NC(=O)OC(C)(C)C)cc1O]

Step 1: To a solution of tert-butyl N-(3-benzyloxy-4-methoxy-phenyl)carbamate (3.00 g, 9.11 mmol, 1.00 eq) in methanol (150 mL) is added wet Pd—C(10%, 0.3 g) under N2. The suspension is degassed under vacuum and purged with H2 several times. The mixture is stirred under H2 (50 psi) at 25° C. for 12 h. The reaction mixture is filtered and concentrated under reduced pressure to afford tert-butyl N-(3-hydroxy-4-methoxy-phenyl)carbamate (2.50 crude as dark oil.

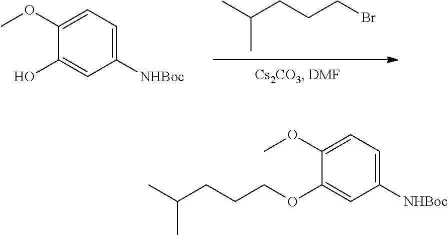

Step 2: To a solution of tert-butyl N-(3-hydroxy-4-methoxy-phenyl)carbamate (2.40 g, 10.0 mmol, 1.00 eq) in dimethylformamide (15 mL) is added cesium carbonate (6.54 g, 20.1 mmol, 2.00 eq) and 1-bromo-4-methyl-pentane (1.82 g, 11.0 mmol, 1.56 mL, 1.10 eq) dropwise. The mixture is stirred at 25° C. for 1.5 h. The reaction mixture is poured into water (50 mL), and extracted with ethyl acetate. The combined organic layers are washed with brine, dried over sodium sulfate, filtered and concentrated under reduced pressure. The residue is purified by column chromatography to afford tert-butyl N-(3-isohexyloxy-4-methoxy-phenyl) carbamate (2.50 g, 7.73 mmol, 77.10% yield) as a white solid.

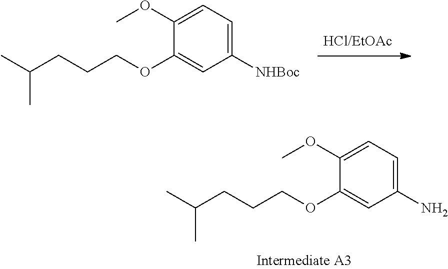 [SMILES: COc1ccc(NC(=O)OC(C)(C)C)cc1OCCCC(C)C ; COc1ccc(N)cc1OCCCC(C)C]

Step 3: A solution of tert-butyl N-(3-isohexyloxy-4-methoxy-phenyl)carbamate (2.50 g, 7.73 mmol, 1.00 eq) in hydrogen chloride (4 Min ethyl acetate, 150 mL) is stirred at 25° C. for 2 h. The reaction mixture is concentrated under reduced pressure, then diluted with aqueous sodium hydrogen carbonate (200 mL) and extracted with ethyl acetate. The combined organic layers are dried over sodium sulfate filtered and concentrated under reduced pressure to afford 3-isohexyloxy-4-methoxy-aniline (1.60 g, 7.16 mmol, 92.7% yield) as dark oil.

### Intermediate A4: 4-isocycanato-1-methoxy-2-((4-methylpenyl)oxy)benzene

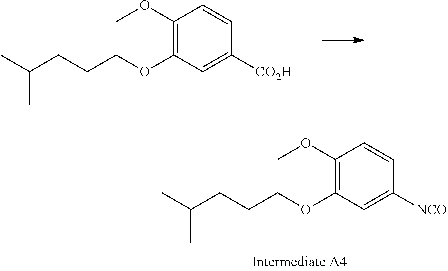

A mixture of 4-Methoxy-3-(((4-methylpentyl)oxy)benzoic acid (12.3 mmol, 3.1 g), diphenyl phosphoryl azide (14.7 mmol, 4.1 g), and triethylamine (17.2 mmol, 1.7 g) in toluene (20 mL) is heated at 70° C. for 12 hours. The mixture is cooled and concentrated under vacuum to give a 4-isocycanato-1-methoxy-2-((4-methylpenyl)oxy)benzene as a crude oil.

### Intermediate A5: 1-(3-hydroxy-4-methoxy-phenyl)-3-[(1R)-2-hydroxy-1-phenyl-ethyl]urea

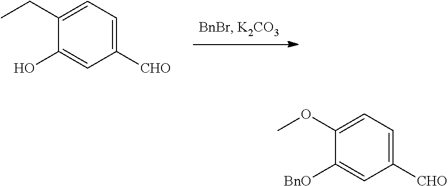 [SMILES: CCc1ccc(C=O)cc1O ; COc1ccc(C=O)cc1OCc1ccccc1]

The mixture of 3-hydroxy-4-methoxy-benzaldehyde (100 g, 657 mmol, 1.0 eq), benzyl bromide (135 g, 788 mmol, 93.7 mL, 1.2 eq), and potassium carbonate (109 g, 788 mmol, 1.2 eq) in methanol (400 mL) is stirred at 70° C. for 12 h. The reaction mixture is filtered and the filter cake is dried under reduced pressure to give 3-(benzyloxy)-4-methoxybenzaldehyde (150 g, 614 mmol, 93.4% yield, 99.1% purity) as a white solid which is used into the next step without further purification.

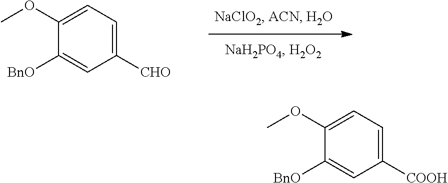 [SMILES: COc1ccc(C=O)cc1OCc1ccccc1 ; COc1ccc(C(=O)O)cc1OCc1ccccc1]

Hydrogen peroxide (24.3 g, 215 mmol, 145 μL, 30% purity, 1.0 eq) is added to a solution of 3-benzyloxy-4-methoxy-benzaldehyde (50.0 g, 206 mmol, 1.0 eq) and disodium dihydrogen pyrophosphate (6.44 g, 53.7 mmol, 0.23 eq) in acetonitrile (650 mL) and water (250 mL) at 25° C. Then the solution is cooled to 0° C., and a solution of sodium chlorite (33.1 g, 289 mmol, 79% purity, 1.4 eq) in water (150 mL) is added. After addition the solution is stirred at 25° C. for 16 h. The reaction mixture is added to a solution of sodium thiosulfate in water (200 ml) and then adjusted pH to 2 with 6 M HCl, filtered to give 3-(benzyloxy)-4-methoxybenzoic acid (50.0 g, 184 mmol, 89.1% yield, 95% purity) as a white solid.

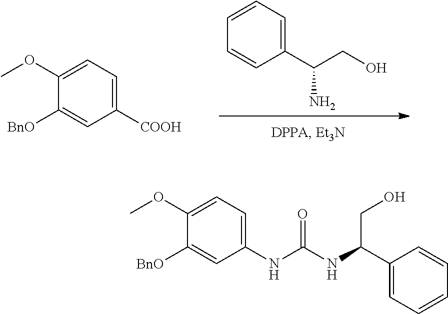 [SMILES: COc1ccc(C(=O)O)cc1OCc1ccccc1 ; N[C@@H](CO)c1ccccc1 ; COc1ccc(NC(=O)N[C@@H](CO)c2ccccc2)cc1OCc1ccccc1]

To a solution of 3-benzyloxy-4-methoxy-benzoic acid (5.00 g, 19.4 mmol, 1.00 eq) in toluene (200 mL) is added triethylamine (3.92 g, 38.7 mmol, 5.37 mL, 2.00 eq) and stirred at 150° C. to remove water by a Dean-Stark apparatus for 2 hrs, then cooled to 85° C. (2R)-2-amino-2-phenyl-ethanol (2.92 g, 21.3 mmol, 1.10 eq) and DPPA (6.39 g, 23.2 mmol, 5.03 mL, 1.20 eq) are added dropwise. The mixture is stirred at 85° C. for 2 hrs. The reaction mixture is concentrated under reduced pressure. 200 mL of water/ethyl acetate (1:1) is added and stirred for 30 min. The solid is collected by filtration. The solid is triturated with ethyl acetate (150 mL) and dried in vacuum to afford 1-(3-benzyloxy-4-methoxy-phenyl)-3-[(1R)-2-hydroxy-1-phenyl-ethyl]urea (4.50 g, crude) as a yellow solid.

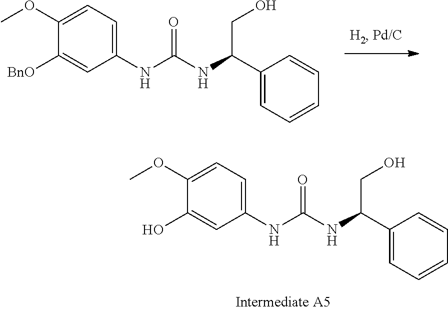 [SMILES: COc1ccc(NC(=O)N[C@@H](CO)c2ccccc2)cc1OCc1ccccc1 ; COc1ccc(NC(=O)N[C@@H](CO)c2ccccc2)cc1O]

To a solution of 1-(3-benzyloxy-4-methoxy-phenyl)-3-[(1R)-2-hydroxy-1-phenyl-ethyl]urea (4.00 g, 10.2 mmol, 1.00 eq) in methanol (200 mL) is added wet Pd/C (10%, 0.4 g) under N2. The suspension is degassed under vacuum and purged with H2 several times. The mixture is stirred under H2 (50 psi) at 25° C. for 12 hours. The solid is filtered off and the filtrate is concentrated under reduced pressure to give 1-(3-hydroxy-4-methoxy-phenyl)-3-[(1R)-2-hydroxy-1-phenyl-ethyl]urea (2.80 g, 9.26 mmol, 90.9% yield) as a dark solid.

### Intermediate A6: Phenyl (R)-(2-((tert-butyldimethylsilyl)oxy)-1-phenylethyl)carbamate

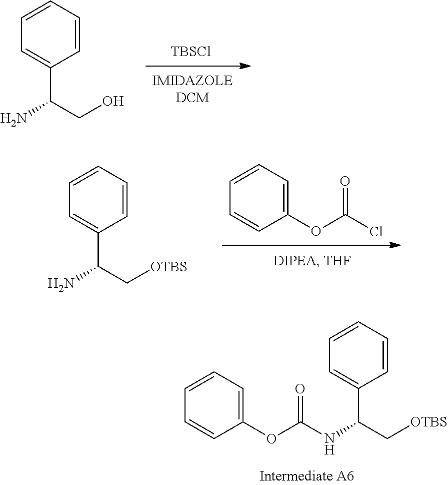 [SMILES: N[C@@H](CO)c1ccccc1 ; "TBSCl" ; CC(C)(C)[Si](C)(C)OC[C@H](N)c1ccccc1 ; O=C(Cl)Oc1ccccc1 ; CC(C)(C)[Si](C)(C)OC[C@H](NC(=O)Oc1ccccc1)c1ccccc1]

Step 1: To a solution of (2R)-2-amino-2-phenyl-ethanol (5.00 g, 36.5 mmol, 1.00 eq) and imidazole (3.72 g, 54.7 mmol, 1.50 eq) in dichloromethane (50 mL) is added TBSCl (6.59 g, 43.7 mmol, 5.36 mL, 1.20 eq). The mixture is stirred at 25° C. for 2 hours. The reaction mixture is diluted with water (20 mL) and extracted with dichloromethane. The combined organic layers are washed with water, dried over sodium sulfate, filtered and concentrated under reduced pressure to give a residue. The crude product is purified by silica gel chromatography eluted with Petroleum ether/Ethyl acetate=10:1 to give product (1R)-2-[tert-butyl (dimethyl)silyl]oxy-1-phenyl-ethanamine (4.20 g, 16.7 mmol, 46% yield) as yellow oil.

Step 2: To a solution of (1R)-2-[tert-butyl(dimethyl)silyl]oxy-1-phenyl-ethanamine (4.20 g, 16.7 mmol, 1.00 eq) and DIPEA (4.32 g, 33.4 mmol, 5.84 mL, 2.00 eq) in tetrahydrofuran (30 mL) is added phenyl chloroformate (3.14 g, 20.0 mmol, 2.51 mL, 1.20 eq) in tetrahydrofuran (20 mL) dropwise. The mixture is stirred at 0° C. for 2 hours. The reaction mixture is diluted with water (30 mL) and extracted with dichloromethane. The combined organic layers are washed with brine, dried over sodium sulfate, filtered and concentrated under reduced pressure to give a residue. The crude product is triturated with Petroleum ether/Ethyl acetate=20/1 to give phenyl N-[(1R)-2-[tert-butyl(dimethyl)silyl] oxy-1-phenyl-ethyl]carbamate (1.80 g, 4.80 mmol, 29% yield, 99.1% purity) as a white solid.

### Intermediate A7: 4-Methoxy-3-(4-methylpent-1-yn-1-yl)benzoic acid

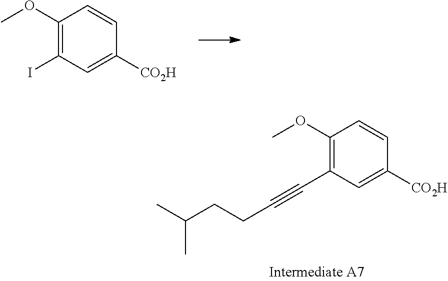 [SMILES: COc1ccc(C(=O)O)cc1I ; COc1ccc(C(=O)O)cc1C#CCCC(C)C]

To a solution of 3-iodo-4-methoxybenzoic acid (17.9 mmol, 5 gms), 4-methylpnent-1-yne (17.9 mmol, 1.7 gms) in acetonitrile (60 mL) is deoxygenated and flooded with nitrogen blanket. Bis(triphenylphosphine)palladium (II) dichloride (0.3 gms), and copper (1) iodide (1.3 g) are added. The resulting slurry is allowed to stir at room temperature for 12 hours. The reaction mixture is filtered. The filtrate is quenched with water then extracted with ethyl acetate. The organics are washed with brine then dried over sodium sulfate, and filtered. The filtrate is concentrated under vacuum to give a crude oil. Purification of the oil is achieved by chromatography on silica using n-hexane as eluant to 4-Methoxy-3-(4-methylpent-1-yn-1-yl)benzoic acid (2.1 g) as a tan solid.

### Intermediate A8: 4-methoxy-3-(5-methylhexyl)benzoic acid

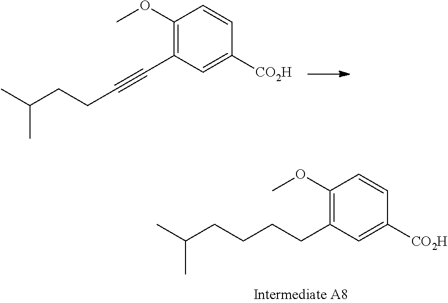 [SMILES: COc1ccc(C(=O)O)cc1C#CCCC(C)C ; COc1ccc(C(=O)O)cc1CCCCC(C)C]

4-Methoxy-3-(4-methylpent-1-yn-1-yl)benzoic acid (4.1 mmol, 1.0 g) is hydrogenated over palladium on carbon (10% by wt.) in methanol at 1 atmosphere. After 2 hours, the reaction is evacuated and flushed with nitrogen, filtered over celite, and the filtrate concentrated under vacuum to give 4-methoxy-3-(5-methylhexyl)benzoic acid (0.8 g) as a white solid.

### Intermediate A9: Phenyl N-(3-isohexyloxy-4-methoxy-phenyl)carbamate

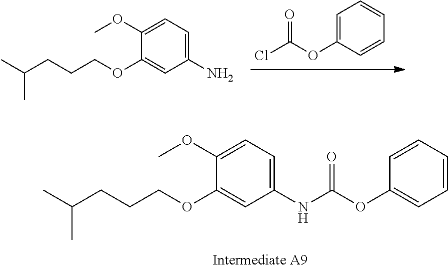 [SMILES: COc1ccc(N)cc1OCCCC(C)C ; O=C(Cl)Oc1ccccc1 ; COc1ccc(NC(=O)Oc2ccccc2)cc1OCCCC(C)C]

To a solution of 3-isohexyloxy-4-methoxy-aniline (200 mg, 896 umol, 1.00 eq) in dichloromethane (5 mL) is added diisopropylethylamine (231 mg, 1.79 mmol, 313 uL, 2.00 eq) and phenyl chloroformate (168.27 mg, 1.07 mmol, 134.62 uL, 1.20 eq) is added at 0° C. The mixture is stirred at 25° C. for 2 h. The reaction mixture is concentrated under reduced pressure to afford phenyl N-(3-isohexyloxy-4-methoxy-phenyl)carbamate (300 mg, crude) as brown oil.

### Intermediate A10: 1-[3-(2-chloroethoxy)-4-methoxy-phenyl]-3-[(1R)-2-hydroxy-1-phenyl-ethyl]urea

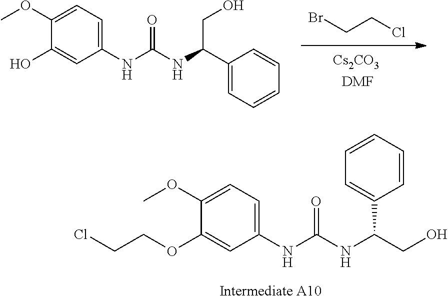

To a solution of 1-(3-hydroxy-4-methoxy-phenyl)-3-[(1R)-2-hydroxy-1-phenyl-ethyl]urea (200 mg, 662 μmol, 1.0 eq) in dimethyl formamide (10 mL) is added cesium carbonate (431 mg, 1.32 mmol, 2.00 eq) and 1-bromo-2-chloro-ethane (114 mg, 794 μmol, 65.8 μL, 1.2 eq). The mixture is stirred at 80° C. for 12 hours. The reaction mixture is diluted with water (20 mL) and extracted with ethyl acetate. The combined organic layers are washed with brine, dried over sodium sulfate, filtered and concentrated under reduced pressure to give a residue. The residue is purified by column chromatography to afford 1-[3-(2-chloroethoxy)-4-methoxy-phenyl]-3-[(1R)-2-hydroxy-1-phenyl-ethyl]urea (150 mg, crude) as a solid.

### Intermediate A11: [(2R)-2-[(3-hydroxy-4-methoxy-phenyl)carbamoylamino]-2-phenyl-ethyl] acetate

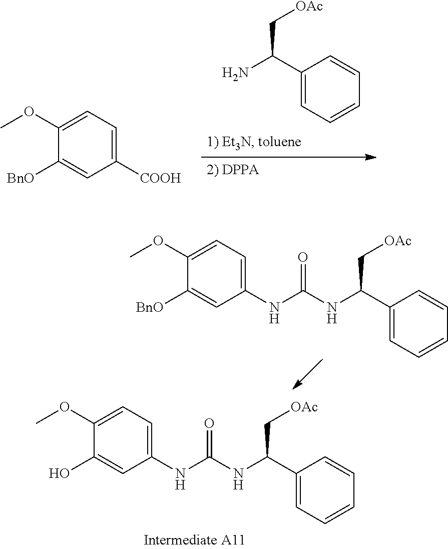 [SMILES: COc1ccc(C(=O)O)cc1OCc1ccccc1 ; CC(=O)OC[C@H](N)c1ccccc1 ; COc1ccc(NC(=O)N[C@@H](COC(C)=O)c2ccccc2)cc1OCc1ccccc1 ; COc1ccc(NC(=O)N[C@@H](COC(C)=O)c2ccccc2)cc1O]

Step 1: To a solution of 5-benzyloxy-6-methoxy-phenyl-3-carboxylic acid and [(2R)-2-amino-2-phenyl-ethyl] acetate (1.2 eq, HCl) in toluene (200 mL) is added triethylamine (3.0 eq). The mixture is refluxed in a Dean-Stack apparatus for 2 hrs to remove water, and then cooled to 85° C. DPPA (1.2 eq) is added drop-wise. The mixture is stirred at 85° C. for 2 hrs. The reaction mixture is concentrated under reduced pressure, then diluted with water (200 mL) and extracted with ethyl acetate (100 mL×3). The combined organic layers are washed with brine, dried over sodium sulfate, filtered and concentrated under reduced pressure to afford [(2R)-2-[(5-benzyloxy-6-methoxy-3-phenyl) carbamoylamino]-2-phenyl-ethyl] acetate.

Step 2: To a solution of [(2R)-2-[(5-benzyloxy-6-methoxy-3-phenyl)carbamoylamino]-2-phenyl-ethyl] acetate (1.00 g, 2.30 mmol, 1.00 eq) in methanol (20 mL) is added Pd/C (200 mg, 10% purity) under nitrogen. The suspension is degassed under vacuum and purged with H2 several times. The mixture is stirred under H2 (50 psi) at 25° C. for 12 hrs. The reaction mixture is filtered and concentrated under reduced pressure to afford [(2R)-2-[(5-hydroxy-6-methoxy-3-phenyl)carbamoylamino]-2-phenyl-ethyl] acetate.

### Intermediate A12: 4-nitrophenyl (4-methoxy-3-((4-methylpentyl)oxy)phenyl)carbamate

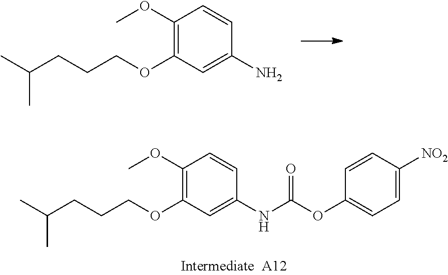 [SMILES: COc1ccc(N)cc1OCCCC(C)C ; COc1ccc(NC(=O)Oc2ccc([N+](=O)[O-])cc2)cc1OCCCC(C)C]

To a solution of p-nitrophenylchloroformate (1.397 g, 6.948 mmol, 1.2 eq) in dry DCM (22 mL) is added a suspension of 4-methoxy-3-((4-methylpentyl)oxy)aniline hydrochloride (1.50 g, 5.79 mmol, 1 eq) and pyridine (0.914 g, 0.93 mL, 11.58 mmol, 2 eq) in 22 ml DCM slowly dropwise at 0° C. The reaction mixture is stirred at room temperature overnight. Upon completion, the reaction mixture is diluted with 100 mL DCM and washed with 5% w/v aqueous citric acid and the DCM layer is dried over anhydrous MgSO4 and concentrated. The crude residue is triturated with ˜10% EtOAc in hexanes (˜40 mL), sonicated, filtered and washed with hexanes to give a white solid (1.968 g, 87%).

### Intermediate A13: 2-(1-amino-2-hydroxyethyl)phenol

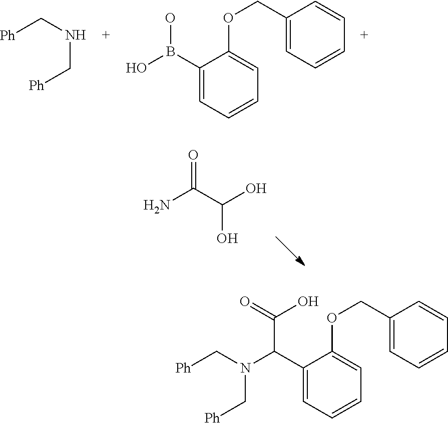 [SMILES: c1ccc(CNCc2ccccc2)cc1 ; OB(O)c1ccccc1OCc1ccccc1 ; NC(=O)C(O)O ; O=C(O)C(c1ccccc1OCc1ccccc1)N(Cc1ccccc1)Cc1ccccc1]

Step 1: To a solution of dibenzylamine (10 mmol, 1.9 gms), (2-(2-benzyloxy)phenyl)boronic acid (10 mmol, 2.3 g) in methanol (10 mL) is added 2,2-dihydroxyacetic acid (10 mmol, 0.9 gms). The resulting solution is allowed to stir at room temperature for 12 hours then concentrated. The mixture is quenched with water and extracted with ethyl acetate. The organics are washed with brine then dried over sodium sulfate, and filtered. The filtrate is concentrated under vacuum to give a crude oil. Purification of the oil is achieved by chromatography on silica using 20-400% ethyl acetate in n-hexane as eluant to give 2-(2-(benzyloxy)phenyl-2-(dibenzylamino) acetic acid weighing (9.4 mmol, 4.1 gms) as a clear oil.

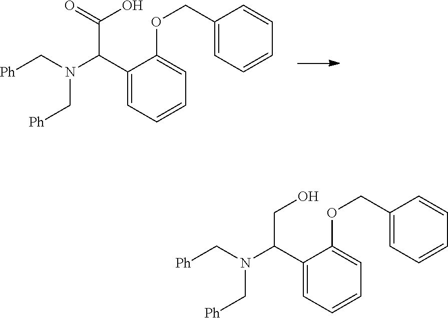 [SMILES: O=C(O)C(c1ccccc1OCc1ccccc1)N(Cc1ccccc1)Cc1ccccc1 ; OCC(c1ccccc1OCc1ccccc1)N(Cc1ccccc1)Cc1ccccc1]

Step 2: Propyl chloroformate (3.4 mmol, 0.4 g) is added to a solution of 2-(2-(benzyloxy)phenyl-2-(dibenzylamino) acetic acid (3.4 mmol, 1.5 g) in dichloromethane (10 mL) at OC (ice bath temperature). Triethylamine (4.1 mmol, 0.4 g) is added and the resulting solution is allowed to warm to room temperature and stir for 30 minutes. The reaction is quenched with water and extracted with dichloromethane. The organics are washed with brine then dried over sodium sulfate, and filtered. The filtrate is concentrated under vacuum to give a crude oil. The crude oil is dissolved in methanol (10 mL) and cooled to 0 C. Sodium borohydride (8.2 mmol, 0.31 g) is added portion wise. After stirring at 0° C. for 30 minutes, the reaction is quenched with water and extracted with dichloromethane. The organics are washed with brine then dried over sodium sulfate, and filtered. The filtrate is concentrated under vacuum to give 2-(2-(benzyloxy)phenyl-2-dibenzylamino)ethan-1-ol (1.8 mmol 0.84 gm) as a clear oil.

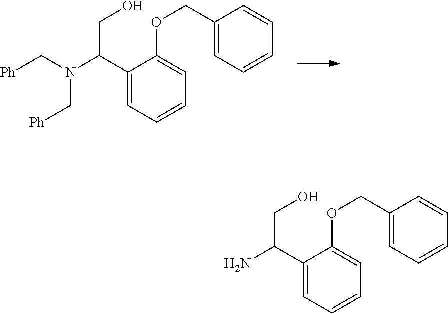 [SMILES: OCC(c1ccccc1OCc1ccccc1)N(Cc1ccccc1)Cc1ccccc1 ; NC(CO)c1ccccc1OCc1ccccc1]

Step 3: Hydrogenated 2-(2-(benzyloxy)phenyl-2-dibenzylamino)ethan-1-ol (1.8 mmol, 0.84 g) over palladium on carbon (10% by wt.) in methanol at 1 atmosphere. After 1 hour, the reaction is evacuated and flushed with nitrogen, filtered over celite, and the filtrate concentrated under vacuum to give 2-(1-amino-2-hydroxyethyl)phenol (0.3 gms) as a clear oil.

### Intermediate A14: [(2R)-2-[(5-hydroxy-6-methoxy-3-pyridyl)carbamoylamino]-2-phenyl-ethyl] acetate

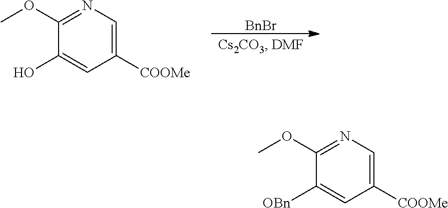 [SMILES: COC(=O)c1cnc(OC)c(O)c1 ; "BnBr" ; COC(=O)c1cnc(OC)c(OCc2ccccc2)c1]

To a solution of methyl 5-hydroxy-6-methoxy-pyridine-3-carboxylate (2.60 g, 14.2 mmol, 1.00 eq) in dimethyl formamide (30 mL) was added cesium carbonate (9.25 g, 28.4 mmol, 2.00 eq) and benzyl bromide (2.91 g, 17.0 mmol, 2.02 mL, 1.20 eq) at 25° C. The mixture was stirred at 25° C. for 30 min. The reaction mixture was poured into water (200 mL) at 0° C., and then extracted with ethyl acetate (90 mL×3). The combined organic layers were washed with aqueous sodium chloride (90 mL×2), dried over sodium sulfate, filtered and concentrated under reduced pressure. The residue was washed with petroleum ether: ethyl acetate=1:1 (10 mL). The solid was concentrated under reduced pressure to afford methyl 5-benzyloxy-6-methoxy-pyridine-3-carboxylate (3.20 g, 11.7 mmol, 82.5% yield) as a white solid.

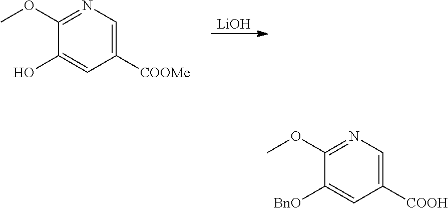 [SMILES: COC(=O)c1cnc(OC)c(O)c1 ; "LiOH" ; COc1ncc(C(=O)O)cc1OCc1ccccc1]

To a solution of methyl 5-benzyloxy-6-methoxy-pyridine-3-carboxylate (3.20 g, 11.7 mmol, 1.00 eq) in tetrahydrofuran (48 mL) and water (5 mL) was added lithium hydroxide mono hydrate (1.40 g, 58.6 mmol, 5.00 eq). The mixture was stirred at 25° C. for 2 hours. The solution was adjust to pH=3˜4 by 1H HCl and extracted with ethyl acetate (30 mL×3). The combined organic layers were washed with brine (50 mL×2), dried over sodium sulfate, filtered and concentrated under reduced pressure to give 5-benzyloxy-6-methoxy-pyridine-3-carboxylic acid (2.80 g, 10.8 mmol, 92% yield) as a white solid.

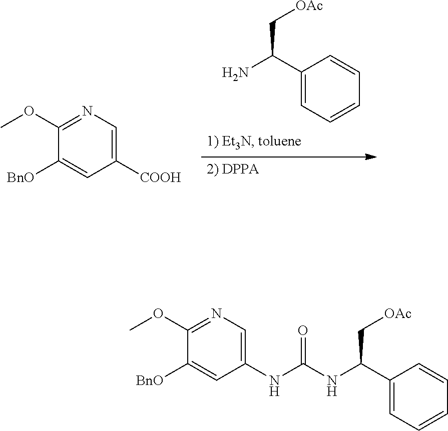 [SMILES: COc1ncc(C(=O)O)cc1OCc1ccccc1 ; CC(=O)OC[C@H](N)c1ccccc1 ; COc1ncc(NC(=O)N[C@@H](COC(C)=O)c2ccccc2)cc1OCc1ccccc1]

To a solution of 5-benzyloxy-6-methoxy-pyridine-3-carboxylic acid (2.50 g, 9.64 mmol, 1.00 eq) and [(2R)-2-amino-2-phenyl-ethyl] acetate (2.49 g, 11.6 mmol, 1.20 eq, HCl) in toluene (200 mL) was added triethylamine (2.93 g, 28.9 mmol, 4.01 mL, 3.00 eq). The mixture was refluxed in a Dean-Stack apparatus for 2 hrs to remove water, and then cooled to 85° C. DPPA (3.18 g, 11.6 mmol, 2.51 mL, 1.20 eq) was added drop-wise. The mixture was stirred at 85° C. for 2 hrs. The reaction mixture was concentrated under reduced pressure, then diluted with water (200 mL) and extracted with ethyl acetate (100 mL 3). The combined organic layers were washed with brine (100 mL×2), dried over sodium sulfate, filtered and concentrated under reduced pressure. The residue was purified by column chromatography (SiO₂, Petroleum ether/Ethyl acetate=3/1 to 0/1) to afford [(2R)-2-[(5-benzyloxy-6-methoxy-3-pyridyl) carbamoylamino]-2-phenyl-ethyl] acetate (1.00 g, 2.30 mmol, 24% yield) as a white solid.

Procedure for Preparation of Intermediate A14:

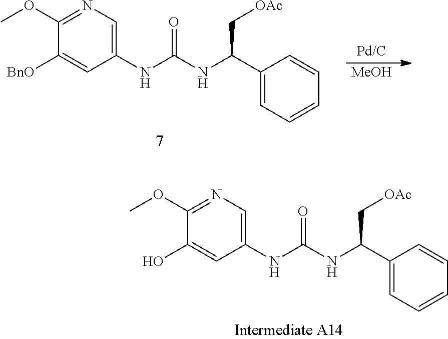 [SMILES: "MeOH" ; COc1ncc(NC(=O)N[C@@H](COC(C)=O)c2ccccc2)cc1OCc1ccccc1 ; COc1ncc(NC(=O)N[C@@H](COC(C)=O)c2ccccc2)cc1O]

To a solution of [(2R)-2-[(5-benzyloxy-6-methoxy-3-pyridyl)carbamoylamino]-2-phenyl-ethyl] acetate (1.00 g, 2.30 mmol, 1.00 eq) in methanol (20 mL) was added Pd/C (200 mg, 10 wt %) under nitrogen. The suspension was degassed under vacuum and purged with H₂ several times. The mixture was stirred under H₂ (50 psi) at 25° C. for 12 hrs. The reaction mixture was filtered and concentrated under reduced pressure to afford [(2R)-2-[(5-hydroxy-6-methoxy-3-pyridyl)carbamoylamino]-2-phenyl-ethyl] acetate (650 mg, 1.88 mmol, 81.8% yield) as a dark solid.

### Example 8. Compound Synthesis

### SM0001 Synthesis

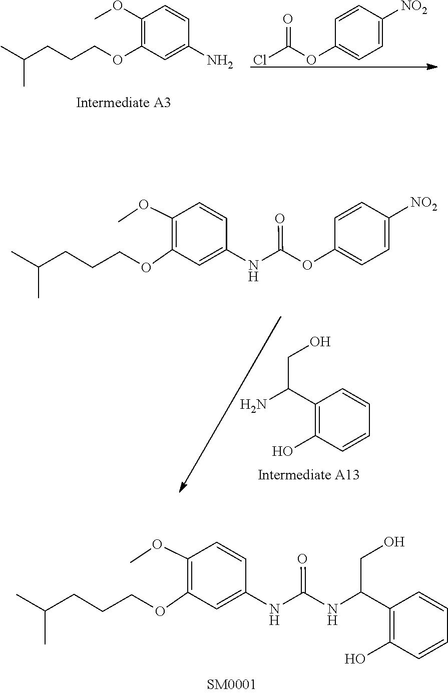 [SMILES: COc1ccc(N)cc1OCCCC(C)C ; O=C(Cl)Oc1ccc([N+](=O)[O-])cc1 ; COc1ccc(N(C)C(=O)Oc2ccc([N+](=O)[O-])cc2)cc1OCCCC(C)C ; NC(CO)c1ccccc1O ; COc1ccc(N(C)C(=O)NC(CO)c2ccccc2O)cc1OCCCC(C)C]

To a stirring slurry of intermediate A3 (4-methoxy-3((4-methylpentyl)oxy)aniline hydrochloride) (0.5 mmol, 0.12 g) and 4-nitrophenyl chloroformate (0.5 mmol, 0.1 g) in dichloromethane (10 mL) at 0° C. is added triethylamine (1.0 mmol, 0.1 g). The resulting solution is stirred at 0° C. for 30 minutes whereupon Intermediate A13 (2-(1-amino-2-hydroxyethyl)phenol, 0.6 mmol, 0.09 g) in dichloromethane (2 mL) is added. The reaction is allowed to stir for 30 minutes then quenched with water and extracted with dichloromethane. The organics are washed with brine then dried over sodium sulfate, and filtered. The filtrate is concentrated under vacuum to give a crude oil. Purification of the oil is achieved using a C18 reverse phase column eluting with 0-100% acetonitrile in water over a 22 minute gradient. The desired fractions are combined and lyophilized to give 1-(2-hydroxy-1-(2-hydroxyphenyl)ethyl)-3-(4-methoxy-3-((4-methylpentyl)oxy)phenyl)urea (0.04 g) as a white solid.

### SM0002 Synthesis

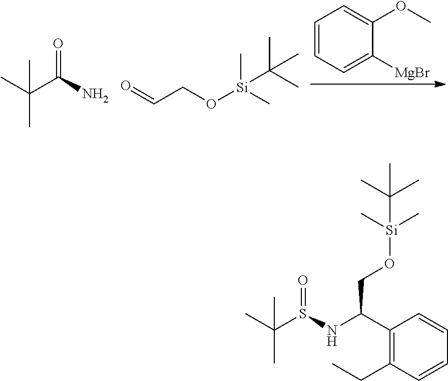

Step 1: A mixture of (R)-2-methylpropane-2-sulfinamide (8.3 mmol, 1 gm), 2-((tert-butyldimethylsilyl)oxy)acetaldehyde (8.3 mmol, 1.4 g), and copper (II) sulfate (16.5 mmol, 2.6 g) in dichloromethane (10 mL) is stirred at room temperature for 24 hours. The mixture is filtered over magnesium sulfate and the filtrate concentrated to give an oil. The resulting oil is dissolved in THF (10 mL) and added drop wise to a stirring solution of (2-methoxyphenyl)magnesium bromide (LOM in THF) (16.4 mL) at −40° C. Reaction mixture is allowed to warm to room temperature and stirred for 1 hour. The reaction is cooled and quenched with water and extracted mixture with dichloromethane. The organics are washed with brine then dried over sodium sulfate, and filtered. The filtrate is concentrated under vacuum to give a crude oil. Purification of the oil is purified by chromatography on silica using 20-40% ethyl acetate in n-hexane as eluant to give (R)—N—((R)-2-((tert-butyldimethylsilyl)oxy-1-(2-methoxyphenyl)ethyl-2-methylpropane-2-sulfinamide (0.42 g) as a yellow solid.

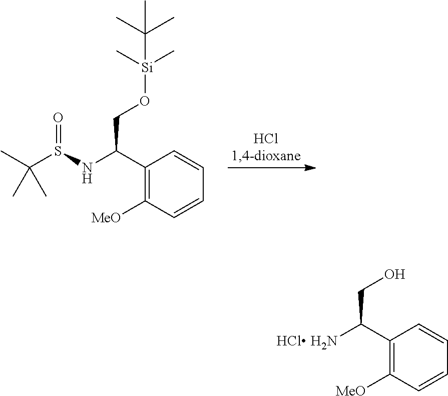

[4N HCl in 1,4-dioxane] (4.32 mL) is added to a stirring solution of (R)—N—((R)-2-((tert-butyldimethylsilyl)oxy-1-(2-methoxyphenyl)ethyl-2-methylpropane-2-sulfinamide (1.1 mmol, 0.42 g) in dichloromethane (10 mL) at 0° C. The resulting mixture is allowed to warm to room temperature and stirred for one hour. The reaction mixture is then concentrated under vacuum to give (R)-2-amino-2-(2-methoxyphenyl)ethan-1-ol hydrochloride (0.15 g) as clear oil.

SM0024 Synthesis and Compounds Synthesized by Similar Procedures

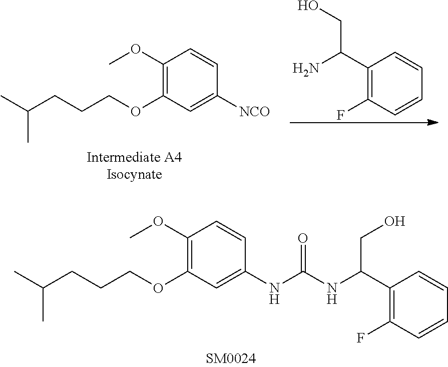

Intermediate A4 (4-isocycanato-1-methoxy-2-((4-methylpenyl)oxy)benzene) (˜0.25 g, 1 mmol) in toluene/triethylamine is let to react with 2-amino-2-(2-fluorophenyl)ethan-1-ol (amine). After 30 minutes the reaction is concentrated in vacuo. Purification is achieved by a Luna 5 uM C18 reverse phase chromatography using a 25-95% ACN (0.1% TFA) in water gradient over 50 minutes. The desired fractions are concentrated to provide the desired product.

The procedure is applied for synthesis of compounds listed in Table 8.

TABLE 8
Compounds synthesized by the procedure described above

| ID# | Structure | (Amine) |
|---|---|---|
| SM0018 | 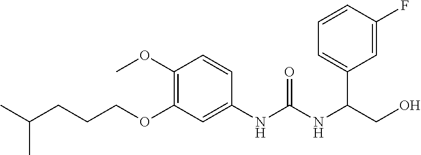 [SMILES: COc1ccc(NC(=O)NC(CO)c2cccc(F)c2)cc1OCCCC(C)C] | 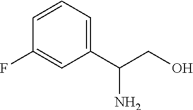 [SMILES: NC(CO)c1cccc(F)c1] |
| SM0003 | 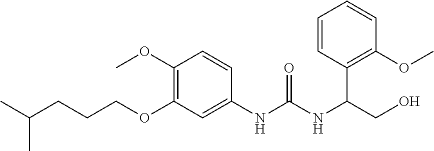 [SMILES: COc1ccc(NC(=O)NC(CO)c2ccccc2OC)cc1OCCCC(C)C] | 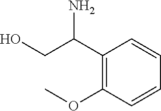 |
| SM0028 | 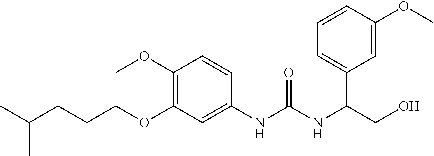 [SMILES: COc1cccc(C(CO)NC(=O)Nc2ccc(OC)c(OCCCC(C)C)c2)c1] | 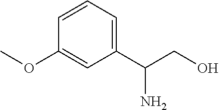 |
| SM0055 | 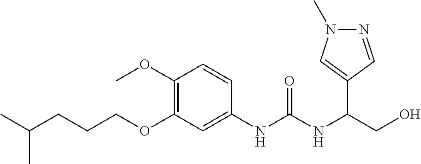 [SMILES: COc1ccc(NC(=O)NC(CO)c2cnn(C)c2)cc1OCCCC(C)C] | 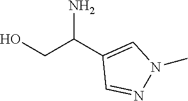 |
| SM0040 | 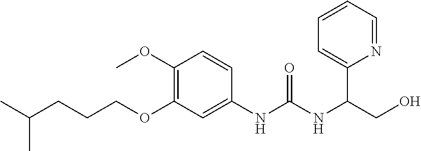 [SMILES: COc1ccc(NC(=O)NC(CO)c2ccccn2)cc1OCCCC(C)C] | 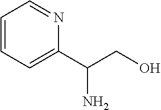 |
| SM0079 | 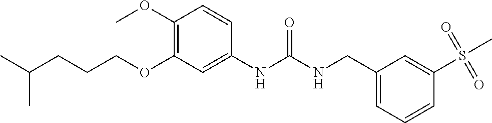 [SMILES: COc1ccc(NC(=O)NCc2cccc(S(C)(=O)=O)c2)cc1OCCCC(C)C] | 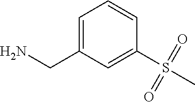 |
| SM0066 | 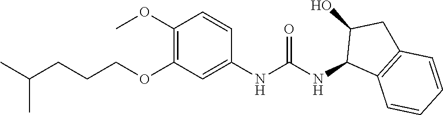 [SMILES: COc1ccc(NC(=O)N[C@@H]2c3ccccc3C[C@@H]2O)cc1OCCCC(C)C] | 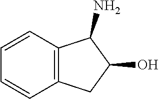 [SMILES: N[C@@H]1c2ccccc2C[C@@H]1O] |
| SM0039 | 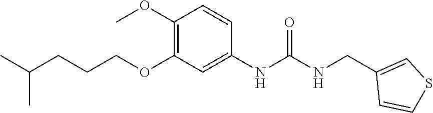 [SMILES: COc1ccc(NC(=O)NCc2ccsc2)cc1OCCCC(C)C] | 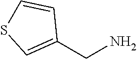 |
| SM0108 | 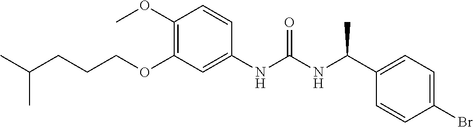 [SMILES: COc1ccc(NC(=O)N[C@@H](C)c2ccc(Br)cc2)cc1OCCCC(C)C] | 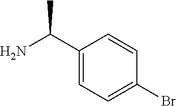 |
| SM0103 | 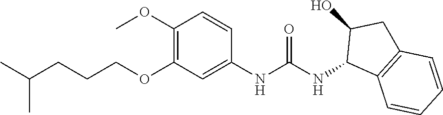 [SMILES: COc1ccc(NC(=O)N[C@H]2c3ccccc3C[C@@H]2O)cc1OCCCC(C)C] | 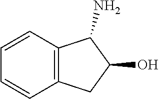 [SMILES: N[C@H]1c2ccccc2C[C@@H]1O] |
| SM0120 | 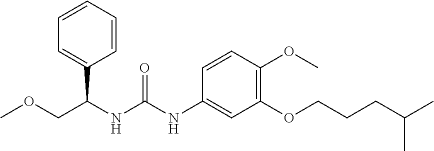 [SMILES: COC[C@H](NC(=O)Nc1ccc(OC)c(OCCCC(C)C)c1)c1ccccc1] | 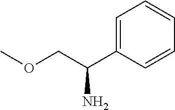 |
| SM0011 | 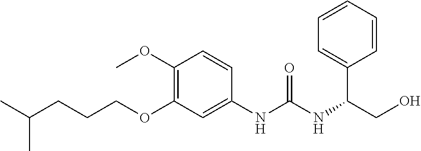 [SMILES: COc1ccc(NC(=O)N[C@@H](CO)c2ccccc2)cc1OCCCC(C)C] | 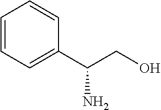 |

### SM0112 Synthesis and Compounds Synthesized by Similar Procedures

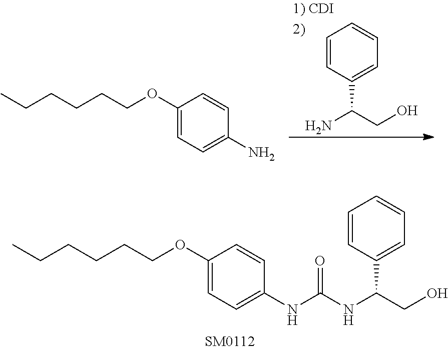 [SMILES: CCCCCCOc1ccc(N)cc1 ; N[C@@H](CO)c1ccccc1 ; CCCCCCOc1ccc(NC(=O)N[C@@H](CO)c2ccccc2)cc1]

1,1′-Carbonyldiimidazole (0.177 g, 1.094 mmol, 1.5 eq) is dissolved in dichloromethane or N,N″-dimethylformamide (2 mL) and 4-(hexyloxy)aniline (aniline) in dichloromethane (2 mL) is added slowly. After stirring at room temperature for 1 h, (R)-2-amino-2-phenylethan-1-ol (amine) is added. The mixture is stirred at room temperature for 1 h. Upon completion of reaction as indicated by LCMS, the solvent is stripped off and the crude purified using reverse phase column chromatography using a gradient of 0-90% acetonitrile in water. The desired fractions are lyophilized to give a white solid.

The procedure is applied for synthesis of compounds listed in Table 9.

TABLE 9
Compounds synthesized by the procedure described above

| ID# | Structure | Aniline | Amine |
|---|---|---|---|
| SM0085 | 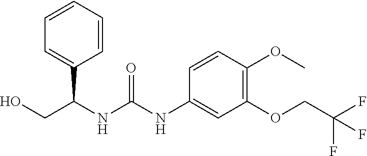 [SMILES: COc1ccc(NC(=O)N[C@@H](CO)c2ccccc2)cc1OCC(F)(F)F] | 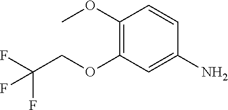 | 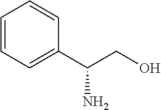 |
| SM0099 | 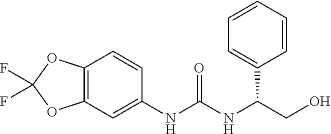 [SMILES: O=C(Nc1ccc2c(c1)OC(F)(F)O2)N[C@@H](CO)c1ccccc1] | 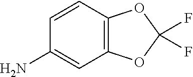 [SMILES: Nc1ccc2c(c1)OC(F)(F)O2] | 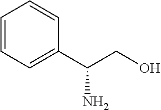 |
| SM0006 | 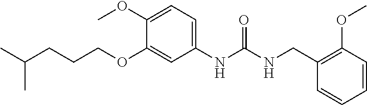 [SMILES: COc1ccccc1CNC(=O)Nc1ccc(OC)c(OCCCC(C)C)c1] | 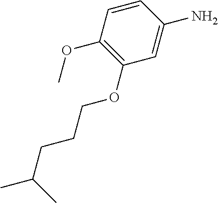 [SMILES: COc1ccc(N)cc1OCCCC(C)C] | 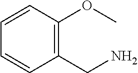 |
| SM0047 | 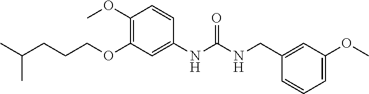 [SMILES: COc1cccc(CNC(=O)Nc2ccc(OC)c(OCCCC(C)C)c2)c1] | 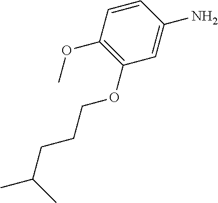 [SMILES: COc1ccc(N)cc1OCCCC(C)C] | 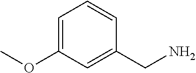 |
| SM0041 | 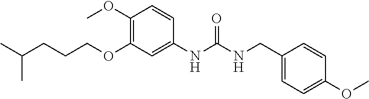 [SMILES: COc1ccc(CNC(=O)Nc2ccc(OC)c(OCCCC(C)C)c2)cc1] | 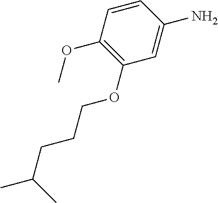 [SMILES: COc1ccc(N)cc1OCCCC(C)C] | 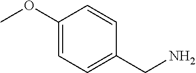 |
| SM0004 | 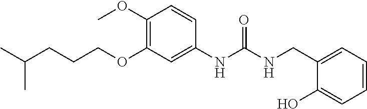 [SMILES: COc1ccc(NC(=O)NCc2ccccc2O)cc1OCCCC(C)C] | 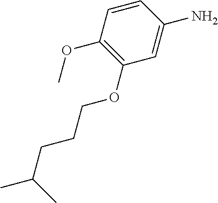 [SMILES: COc1ccc(N)cc1OCCCC(C)C] | 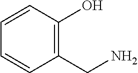 |
| SM0021 | 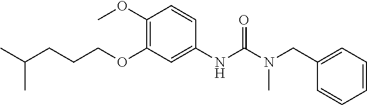 [SMILES: COc1ccc(NC(=O)N(C)Cc2ccccc2)cc1OCCCC(C)C] | 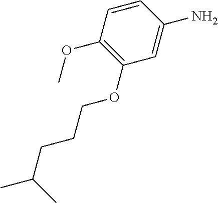 [SMILES: COc1ccc(N)cc1OCCCC(C)C] | 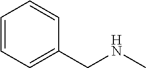 |
| SM0076 | 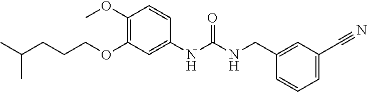 [SMILES: COc1ccc(NC(=O)NCc2cccc(C#N)c2)cc1OCCCC(C)C] | 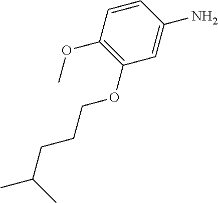 [SMILES: COc1ccc(N)cc1OCCCC(C)C] | 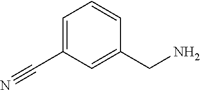 |
| SM100 | 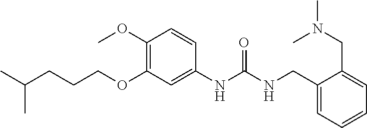 [SMILES: COc1ccc(NC(=O)NCc2ccccc2CN(C)C)cc1OCCCC(C)C] | 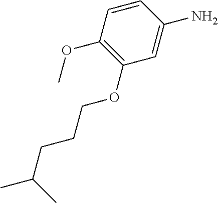 [SMILES: COc1ccc(N)cc1OCCCC(C)C] | 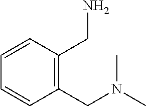 |
| SM0048 | 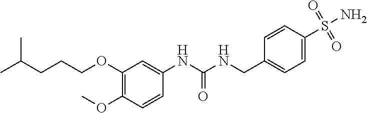 [SMILES: COc1ccc(NC(=O)NCc2ccc(S(N)(=O)=O)cc2)cc1OCCCC(C)C] | 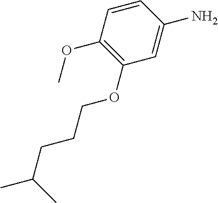 [SMILES: COc1ccc(N)cc1OCCCC(C)C] | 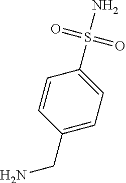 [SMILES: NCc1ccc(S(N)(=O)=O)cc1] |
| SM0063 | 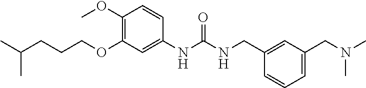 [SMILES: COc1ccc(NC(=O)NCc2cccc(CN(C)C)c2)cc1OCCCC(C)C] | 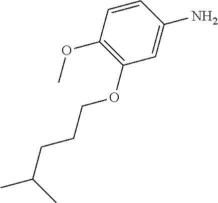 [SMILES: COc1ccc(N)cc1OCCCC(C)C] | 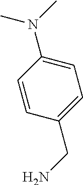 |
| SM0113 | 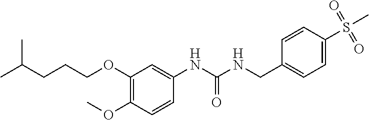 [SMILES: COc1ccc(NC(=O)NCc2ccc(S(C)(=O)=O)cc2)cc1OCCCC(C)C] | 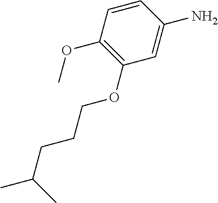 [SMILES: COc1ccc(N)cc1OCCCC(C)C] | 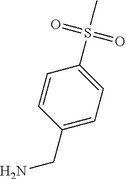 |
| SM0058 | 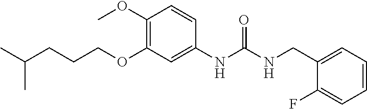 [SMILES: COc1ccc(NC(=O)NCc2ccccc2F)cc1OCCCC(C)C] | 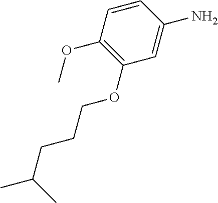 [SMILES: COc1ccc(N)cc1OCCCC(C)C] | 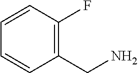 |
| SM0052 | 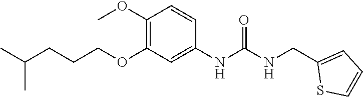 [SMILES: COc1ccc(NC(=O)NCc2cccs2)cc1OCCCC(C)C] | 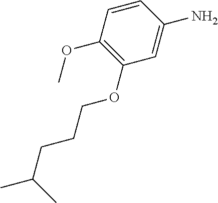 [SMILES: COc1ccc(N)cc1OCCCC(C)C] | 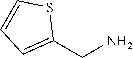 |
| SM0080 | 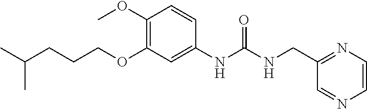 [SMILES: COc1ccc(NC(=O)NCc2cnccn2)cc1OCCCC(C)C] | 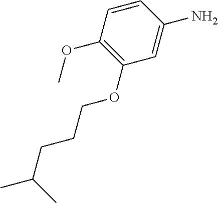 [SMILES: COc1ccc(N)cc1OCCCC(C)C] | 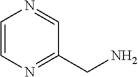 |
| SM0050 | 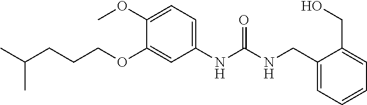 [SMILES: COc1ccc(NC(=O)NCc2ccccc2CO)cc1OCCCC(C)C] | 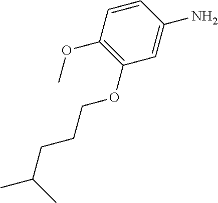 [SMILES: COc1ccc(N)cc1OCCCC(C)C] | 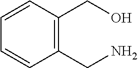 |
| SM0059 | 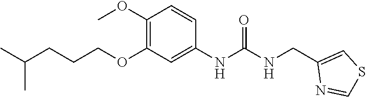 [SMILES: COc1ccc(NC(=O)NCc2cscn2)cc1OCCCC(C)C] | 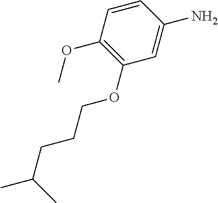 [SMILES: COc1ccc(N)cc1OCCCC(C)C] | 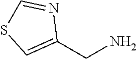 |
| SM0032 | 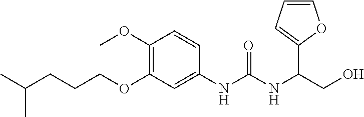 [SMILES: COc1ccc(NC(=O)NC(CO)c2ccco2)cc1OCCCC(C)C] | 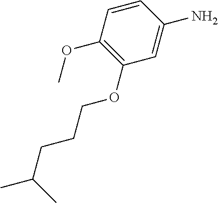 [SMILES: COc1ccc(N)cc1OCCCC(C)C] | 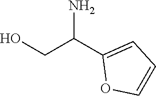 |
| SM0044 | 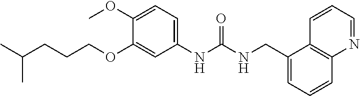 [SMILES: COc1ccc(NC(=O)NCc2cccc3ncccc23)cc1OCCCC(C)C] | 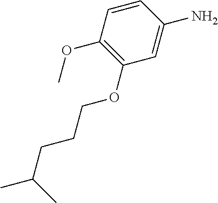 [SMILES: COc1ccc(N)cc1OCCCC(C)C] | 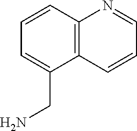 [SMILES: NCc1cccc2ncccc12] |
| SM0104 | 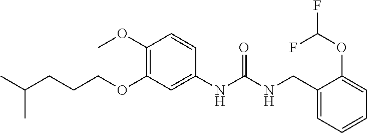 [SMILES: COc1ccc(NC(=O)NCc2ccccc2OC(F)F)cc1OCCCC(C)C] | 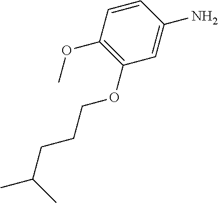 [SMILES: COc1ccc(N)cc1OCCCC(C)C] | 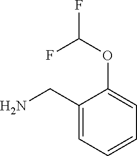 [SMILES: NCc1ccccc1OC(F)F] |

### SM0026 Synthesis and Compounds Synthesized by Similar Procedures

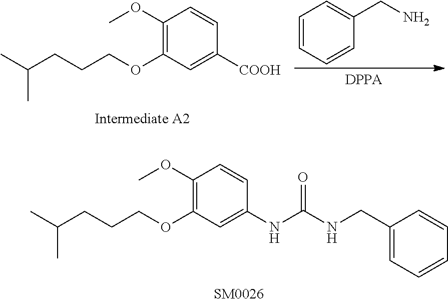 [SMILES: COc1ccc(C(=O)O)cc1OCCCC(C)C ; NCc1ccccc1 ; COc1ccc(NC(=O)NCc2ccccc2)cc1OCCCC(C)C]

To a solution of intermediate A2 (1 eq, 400 mg, 1.58 mmol) in toluene (5 mL) is added triethylamine (1.4 eq, 310 μL, 2.22 mmol) and diphenylphosphoryl azide (DPPA) (1.2 eq, 409 μL, 1.90 mmol). The reaction is stirred for three hours at room temperature, then heated at reflux for 2 hours. The reaction mixture is washed with saturated ammonia chloride solution and water, dried over magnesium sulfate, concentrated in vacuo. Phenylmethanamine (amine) is added to the isocyanate crude reaction. Purified on C18 Flash 5-95° % MeCN.

The procedure is applied for synthesis of compounds listed in Table 10.

TABLE 10
Compounds synthesized by the procedure described above

| ID# | Structure | Intermediate | Amine |
|---|---|---|---|
| SM0009 | 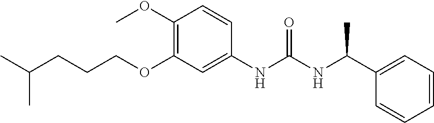 [SMILES: COc1ccc(NC(=O)N[C@@H](C)c2ccccc2)cc1OCCCC(C)C] | A2 | 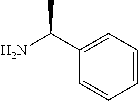 |
| SM0043 | 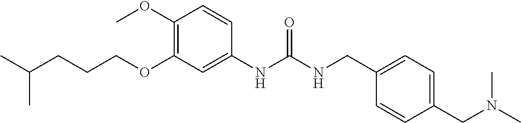 [SMILES: COc1ccc(NC(=O)NCc2ccc(CN(C)C)cc2)cc1OCCCC(C)C] | A2 | 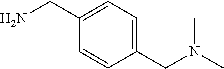 |
| SM0037 | 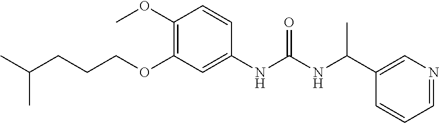 [SMILES: COc1ccc(NC(=O)NC(C)c2cccnc2)cc1OCCCC(C)C] | A2 | 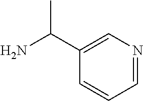 |
| SM0031 | 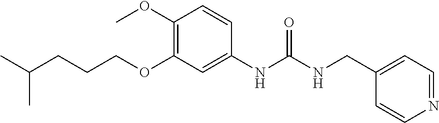 [SMILES: COc1ccc(NC(=O)NCc2ccncc2)cc1OCCCC(C)C] | A2 | 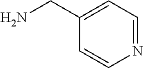 |
| SM0049 | 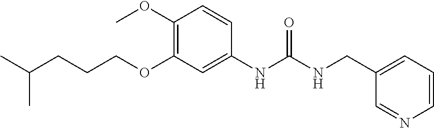 [SMILES: COc1ccc(NC(=O)NCc2cccnc2)cc1OCCCC(C)C] | A2 | 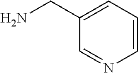 |
| SM0045 | 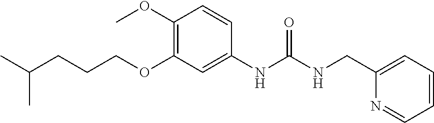 [SMILES: COc1ccc(NC(=O)NCc2ccccn2)cc1OCCCC(C)C] | A2 | 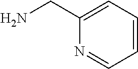 |
| SM0029 | 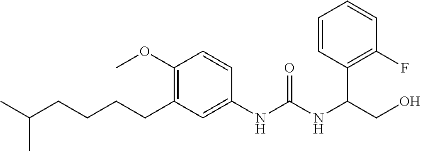 [SMILES: COc1ccc(NC(=O)NC(CO)c2ccccc2F)cc1CCCCC(C)C] | A8 | 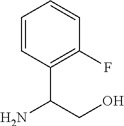 [SMILES: NC(CO)c1ccccc1F] |
| SM0005 | 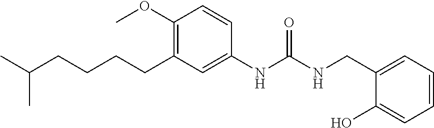 [SMILES: COc1ccc(NC(=O)NCc2ccccc2O)cc1CCCCC(C)C] | A8 | 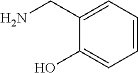 |

### SM0007 Synthesis

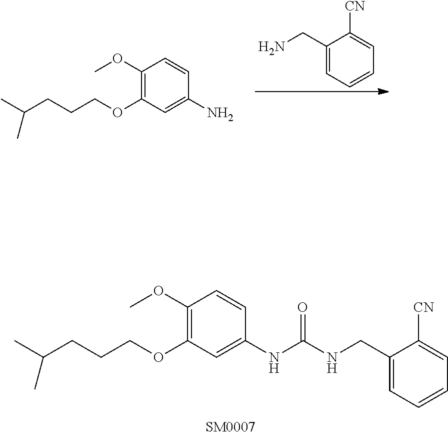 [SMILES: COc1ccc(N)cc1OCCCC(C)C ; N#Cc1ccccc1CN ; COc1ccc(NC(=O)NCc2ccccc2C#N)cc1OCCCC(C)C]

1,1′-Carbonyldiimidazole (0.113 g, 0.695 mmol, 1.2 eq) is dissolved in dimethylformamide (DMF) (2 mL) and a solution of 4-methoxy-3-((4-methylpentyl)oxy)aniline hydrochloride (0.150 g, 0.579 mmol, 1 eq) and pyridine (0.092 g, 0.094 mL, 1.16 mmol, 2 eq) in DMF (1 mL) is added slowly at 0° C. Mixture is allowed to stir at room temperature for 1 h. Solid 2-(aminomethyl) benzonitrile hydrochloride (0.097 g, 0.579 mmol, 1 eq) is added in portions to the reaction mixture. Reaction is stirred at room temperature for 1 h. Upon completion of reaction, as indicated by LCMS, the solvent is stripped off and the crude is purified using reverse phase column chromatography using a gradient of 0-95% acetonitrile in water. The desired fractions are lyophilized to give a white solid (0.103 g, 47%).

### SM0071 Synthesis and Compounds Synthesized by Similar Procedures

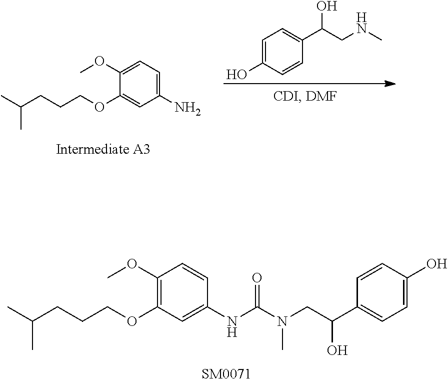 [SMILES: COc1ccc(N)cc1OCCCC(C)C ; CNCC(O)c1ccc(O)cc1 ; COc1ccc(NC(=O)N(C)CC(O)c2ccc(O)cc2)cc1OCCCC(C)C]

To a solution of Intermediate A3 (3-isohexyloxy-4-methoxy-aniline) (100 mg, 448 μmol, 1.0 eq) in dichloromethane (3.00 mL) is added 1,1′-carbonyldiimidazole (CDI, 76.2 mg, 470 μmol, 1.05 eq). The mixture is stirred at 25 CC for 30 mi. Then (Amine, R—NH—R′, 4-[1-hydroxy-2-(methylamino)ethyl]phenol) (74.9 mg, 448 μmol, 1.0 eq) is added. The mixture is stirred at 25° C. for 2 h. The reaction mixture is diluted with water and extracted with dichloromethane. The combined organic layers are dried over sodium sulfate, filtered and concentrated under reduced pressure. The residue is purified by prep-HPLC and lyophilized to afford 1-[2-hydroxy-2-(4-hydroxyphenyl)ethyl]-3-(3-isohexyloxy-4-methoxy-phenyl)-1-methyl-urea (102.56 mg, 246 μmol, 55.0% yield, 1000% purity) as a white solid.

The procedure was applied for synthesis of compounds listed in Table 11.

TABLE 11
Compounds synthesized by the procedure described above

| ID# | Structure | Amine |
|---|---|---|
| SM0057 | 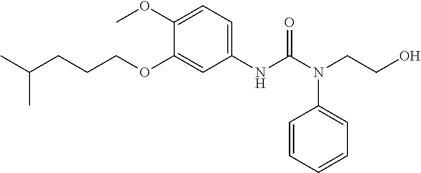 [SMILES: COc1ccc(NC(=O)N(CCO)c2ccccc2)cc1OCCCC(C)C] | 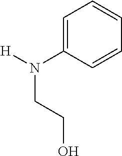 |
| SM0077 | 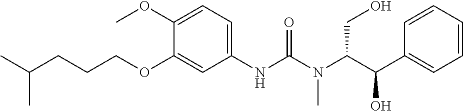 [SMILES: COc1ccc(NC(=O)N(C)[C@H](CO)[C@H](O)c2ccccc2)cc1OCCCC(C)C] | 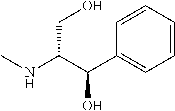 |
| SM0074 | 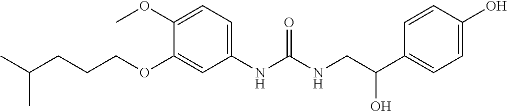 [SMILES: COc1ccc(NC(=O)NCC(O)c2ccc(O)cc2)cc1OCCCC(C)C] | 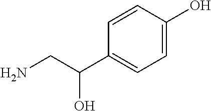 [SMILES: NCC(O)c1ccc(O)cc1] |
| SM0109 | 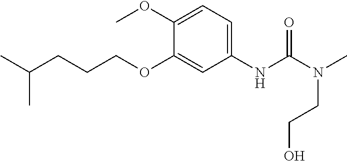 [SMILES: COc1ccc(NC(=O)N(C)CCO)cc1OCCCC(C)C] | 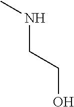 |
| SM0118 | 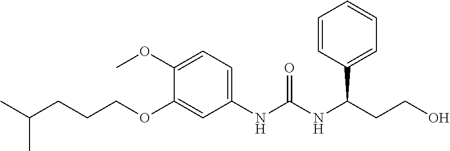 [SMILES: COc1ccc(NC(=O)N[C@H](CCO)c2ccccc2)cc1OCCCC(C)C] | 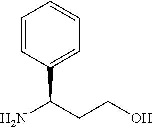 [SMILES: N[C@H](CCO)c1ccccc1] |
| SM0078 | 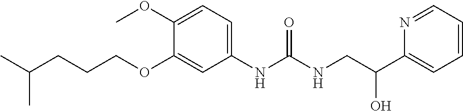 [SMILES: COc1ccc(NC(=O)NCC(O)c2ccccn2)cc1OCCCC(C)C] | 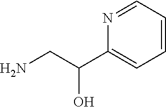 |
| SM0008 | 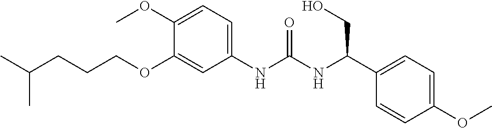 [SMILES: COc1ccc([C@H](CO)NC(=O)Nc2ccc(OC)c(OCCCC(C)C)c2)cc1] | 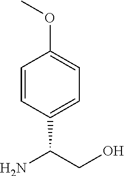 |
| SM0012 | 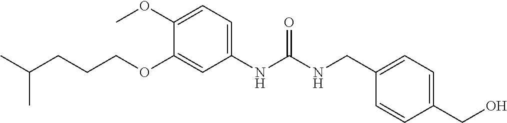 [SMILES: COc1ccc(NC(=O)NCc2ccc(CO)cc2)cc1OCCCC(C)C] | 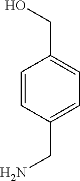 |
| SM0020 | 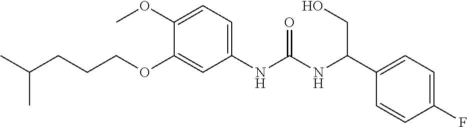 [SMILES: COc1ccc(NC(=O)NC(CO)c2ccc(F)cc2)cc1OCCCC(C)C] | 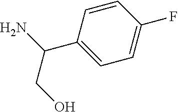 [SMILES: NC(CO)c1ccc(F)cc1] |
| SM0065 | 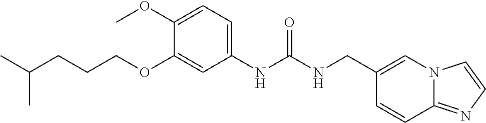 [SMILES: COc1ccc(NC(=O)NCc2ccc3nccn3c2)cc1OCCCC(C)C] | 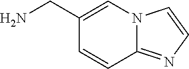 [SMILES: NCc1ccc2nccn2c1] |
| SM0117 | 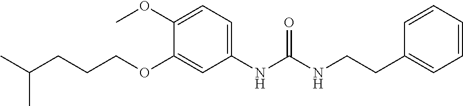 [SMILES: COc1ccc(NC(=O)NCCc2ccccc2)cc1OCCCC(C)C] | 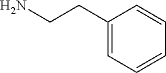 |
| SM0081 | 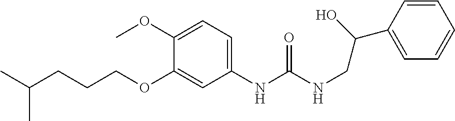 [SMILES: COc1ccc(NC(=O)NCC(O)c2ccccc2)cc1OCCCC(C)C] | 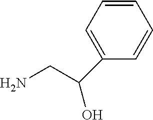 |
| SM0016 | 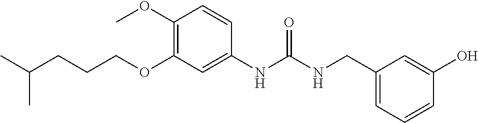 [SMILES: COc1ccc(NC(=O)NCc2cccc(O)c2)cc1OCCCC(C)C] | 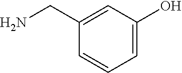 |
| SM0017 | 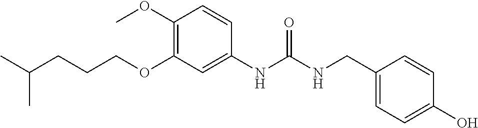 [SMILES: COc1ccc(NC(=O)NCc2ccc(O)cc2)cc1OCCCC(C)C] | 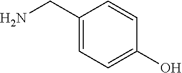 |
| SM0097 | 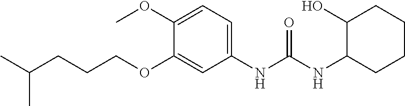 [SMILES: COc1ccc(NC(=O)NC2CCCCC2O)cc1OCCCC(C)C] | 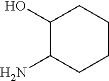 |
| SM0056 | 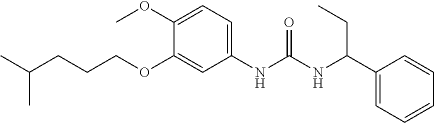 [SMILES: CCC(NC(=O)Nc1ccc(OC)c(OCCCC(C)C)c1)c1ccccc1] | 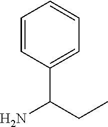 |
| SM0064 | 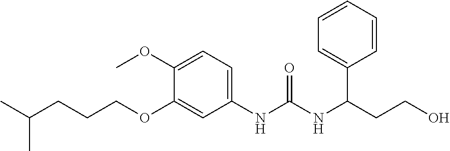 [SMILES: COc1ccc(NC(=O)NC(CCO)c2ccccc2)cc1OCCCC(C)C] | 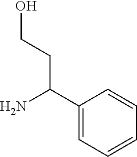 [SMILES: NC(CCO)c1ccccc1] |

### SM0121 Synthesis and Compounds Synthesized by Similar Procedures

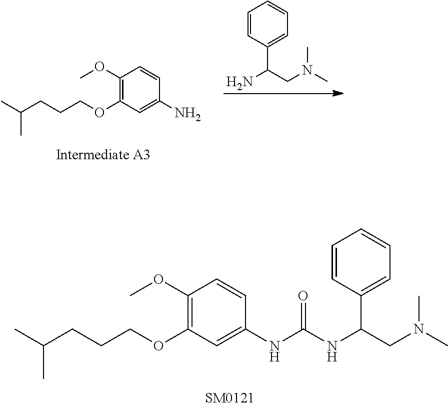 [SMILES: COc1ccc(N)cc1OCCCC(C)C ; CN(C)CC(N)c1ccccc1 ; COc1ccc(NC(=O)NC(CN(C)C)c2ccccc2)cc1OCCCC(C)C]

To a solution of p-nitrobenzylchloroformate (1.1 eq, 91 mg, 0.49 mmol) in DCM (5 mL) is added 4-methoxy-3-((4-methylpentyl)oxy)aniline (aniline) (1 eq, 100 mg, 0.45 mmol) in DCM (1 mL). The reaction is stirred for 5 minutes. N¹,N¹-dimethyl-2-phenylethane-1,2-diamine is then added, neat, to the solution and reaction is stirred for 15 minutes. The reaction is monitored by LCMS. Upon formation of product, reaction is quenched with water then extracted into ethyl acetate. Washed organic phase with water until aqueous phase is clear, dried over magnesium sulfate, reduced the organics under evaporative pressure to yield a yellow oil. Purified on reverse phase flash chromatography to yield product.

The procedure is applied for synthesis of compounds listed in Table 12.

TABLE 12
Compounds synthesized by the procedure described above

| ID# | Structure | Amine |
|---|---|---|
| SM0027 | 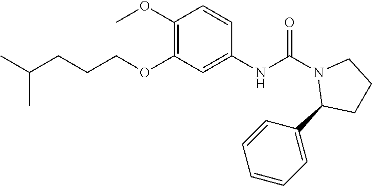 [SMILES: COc1ccc(NC(=O)N2CCC[C@H]2c2ccccc2)cc1OCCCC(C)C] | 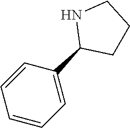 [SMILES: c1ccc([C@@H]2CCCN2)cc1] |
| SM0013 | 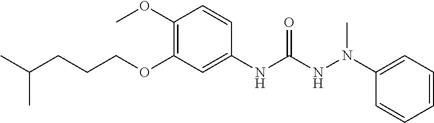 [SMILES: COc1ccc(NC(=O)NN(C)c2ccccc2)cc1OCCCC(C)C] | 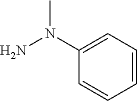 |
| SM0086 | 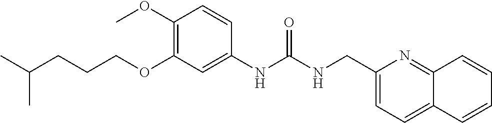 [SMILES: COc1ccc(NC(=O)NCc2ccc3ccccc3n2)cc1OCCCC(C)C] | 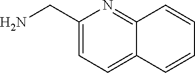 [SMILES: NCc1ccc2ccccc2n1] |
| SM0022 | 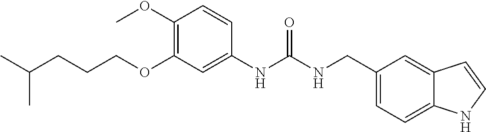 [SMILES: COc1ccc(NC(=O)NCc2ccc3[nH]ccc3c2)cc1OCCCC(C)C] | 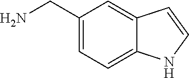 [SMILES: NCc1ccc2[nH]ccc2c1] |
| SM0106 | 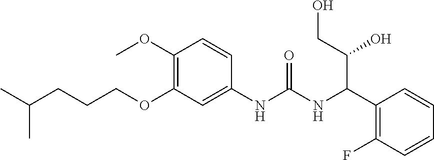 [SMILES: COc1ccc(NC(=O)NC(c2ccccc2F)[C@@H](O)CO)cc1OCCCC(C)C] | 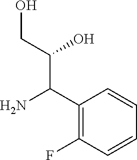 [SMILES: NC(c1ccccc1F)[C@@H](O)CO] |
| SM0082 | 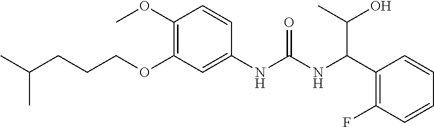 [SMILES: COc1ccc(NC(=O)NC(c2ccccc2F)C(C)O)cc1OCCCC(C)C] | 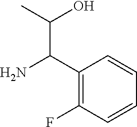 |
| SM0036 | 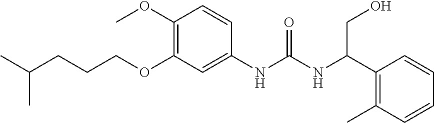 [SMILES: COc1ccc(NC(=O)NC(CO)c2ccccc2C)cc1OCCCC(C)C] | 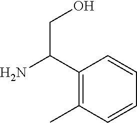 |
| SM0102 | 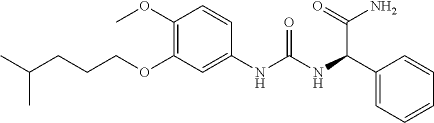 [SMILES: COc1ccc(NC(=O)N[C@@H](C(N)=O)c2ccccc2)cc1OCCCC(C)C] | 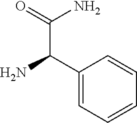 [SMILES: NC(=O)[C@H](N)c1ccccc1] |
| SM0030 | 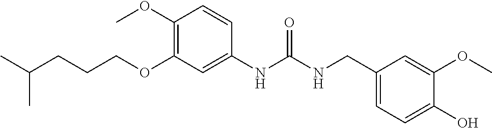 [SMILES: COc1cc(CNC(=O)Nc2ccc(OC)c(OCCCC(C)C)c2)ccc1O] | 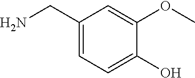 |
| SM0023 | 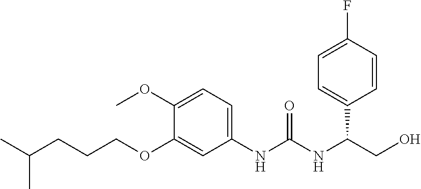 [SMILES: COc1ccc(NC(=O)N[C@@H](CO)c2ccc(F)cc2)cc1OCCCC(C)C] | 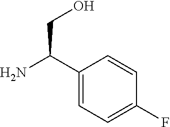 [SMILES: N[C@@H](CO)c1ccc(F)cc1] |

### (R)-2-amino-2-phenylacetamide synthesis

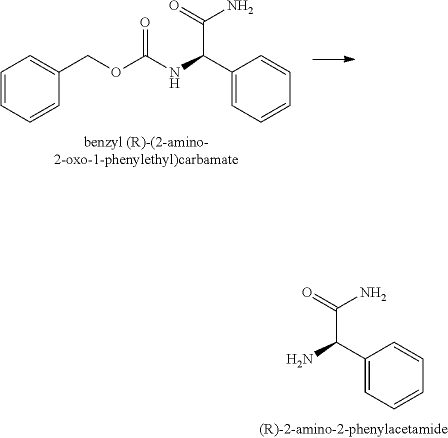 [SMILES: NC(=O)[C@H](NC(=O)OCc1ccccc1)c1ccccc1 ; NC(=O)[C@H](N)c1ccccc1]

(R)-2-amino-2-phenylacetamide, used in synthesis of Compound SM0102, is synthesized by hydrogenation of benzyl (R)-(2-amino-2-oxo-1-phenylethyl)carbamate (0.7 mmol, 0.21 gm) over palladium on carbon (10% by wt.) in methanol at 1 atmosphere H₂ (gas). After 1 hour, the reaction is evacuated and flushed with nitrogen, filtered over celite, and the filtrate concentrated under vacuum to give (R)-2-amino-2-phenylacetamide (0.1 g) as a clear oil.

### SM0014 and SM0046 Synthesis

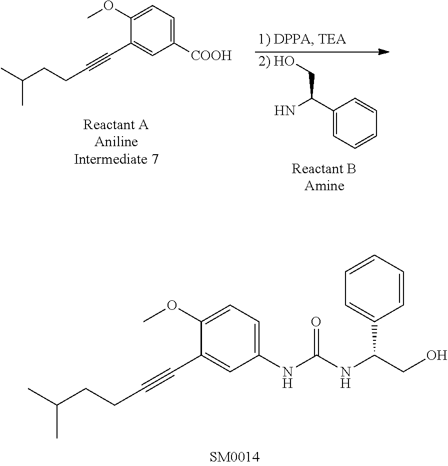

A mixture of Intermediate A7 (4-Methoxy-3-(4-methylpent-1-yn-1-yl)benzoic acid (1 EQ), diphenyl phosphoryl azide (1.2 EQ), and triethylamine (1.4 EQ) in toluene (20 mL) is heated at 70° C. for 12 hours. The mixture is cooled and concentrated under vacuum to give 4-isocycanato-1-methoxy-2-((4-methylpenyl)oxy)benzene as a crude oil.

To a solution of CDI (1 eq, 41 mg, 0.25 mmol) in DMF (500 μL), a solution of Reactant A, aniline, (1 eq) in DCM (3 mL) was added. The reaction is allowed to stir at room temperature for 30 minutes, monitored via TLC (1:4 hexanes:ethyl acetate). Upon full activation of the aniline, a solution of Reactant B (1 eq) in DCM (1 mL) is added and the reaction is stirred at room temperature for 15 minutes. The reaction is monitored via TLC (1:1 hexanes:ethyl acetate). The reaction is concentrated in vacuo and then purified on reverse phase C18 flash chromatography (5-95% MeCN).

The procedure is applied for synthesis of the compound listed in Table 13.

TABLE 13
Compounds synthesized by the procedure described above

| | | Reactant A | Reactant B |
|---|---|---|---|
| ID# | Structure | (Aniline) | (Amine) |
| SM0046 | 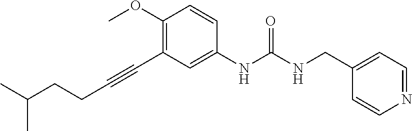 [SMILES: COc1ccc(NC(=O)NCc2ccncc2)cc1C#CCCC(C)C] | 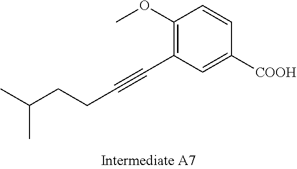 [SMILES: COc1ccc(C(=O)O)cc1C#CCCC(C)C] | 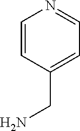 |

### SM0054 Synthesis and Compounds Synthesized by Similar Procedures

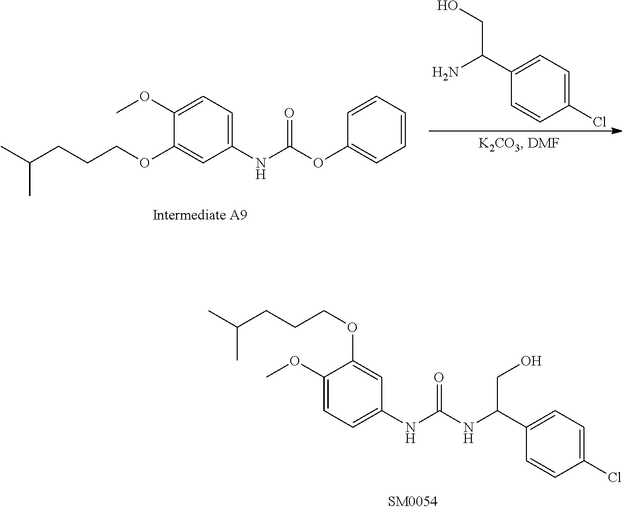 [SMILES: COc1ccc(NC(=O)Oc2ccccc2)cc1OCCCC(C)C ; NC(CO)c1ccc(Cl)cc1 ; COc1ccc(NC(=O)NC(CO)c2ccc(Cl)cc2)cc1OCCCC(C)C]

To a solution of Intermediate A9 (phenyl N-(3-isohexyloxy-4-methoxy-phenyl)carbamate) (100 mg, 291 μmol, 1.0 eq) in dimethylformamide (5 mL) is added potassium carbonate (80.5 mg, 582 μmol, 2.0 eq) and the amine, 4-(aminomethyl)benzene-1,2-diol (56.3 mg, 320 μmol, 1.10 eq, hydrochloric acid). The mixture is stirred at 80° C. for 2 hrs. The reaction mixture is poured into water (10 mL) and extracted with ethyl acetate. The combined organic layers are washed with brine, dried over sodium sulfate, filtered and concentrated under reduced pressure, the residue is purified by prep-HPLC and lyophilized to afford 1-[(3,4-dihydroxyphenyl)methyl]-3-(3-isohexyloxy-4-methoxy-phenyl)urea (31.16 mg, 77.6 μmol, 26.6% yield, 96.7% purity) as a brown solid.

The procedure is applied for synthesis of compounds listed in Table 14.

TABLE 14
Compounds synthesized by the procedure described above

| ID# | Structure | Amine |
|---|---|---|
| SM0015 | 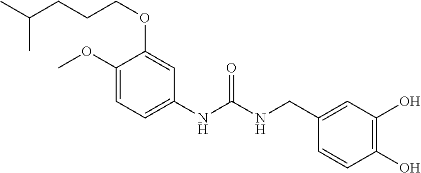 [SMILES: COc1ccc(NC(=O)NCc2ccc(O)c(O)c2)cc1OCCCC(C)C] | 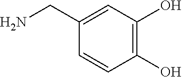 |
| SM0111 | 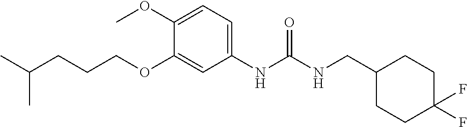 [SMILES: COc1ccc(NC(=O)NCC2CCC(F)(F)CC2)cc1OCCCC(C)C] | 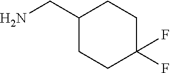 |

### SM0034 Synthesis and Compounds Synthesized by Similar Procedures

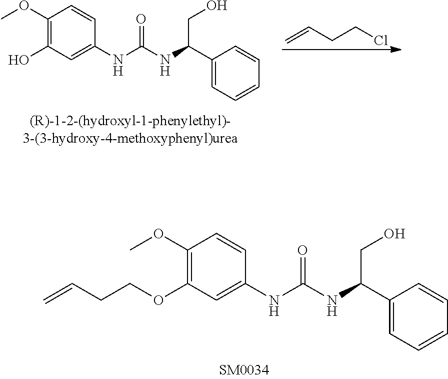

A slurry of (R)-1-2-(hydroxyl-1-phenylethyl)-3-(3-hydroxy-4-methoxyphenyl)urea, 4-chlorobut-1-ene, the alkyl chloride, potassium carbonate, and 18-Crown-6 (cat.) in THF is heated at 40° C. for several hours then cooled. The mixture is quenched with water and extracted with dichloromethane. The organics are washed with brine then dried over sodium sulfate, and filtered. The filtrate is concentrated under vacuum to give a crude oil. Purification of the oil is achieved by using a C18 reverse phase column eluting with 0-100% acetonitrile in water over a 22 minute gradient. The desired fractions are combined and lyophilized to give Compound SM0034 as a white solid.

The procedure is applied for synthesis of compounds listed in Table 15.

TABLE 15
Compounds synthesized by the procedure described above

| ID# | Structure | Alkyl halide |
|---|---|---|
| SM0083 | 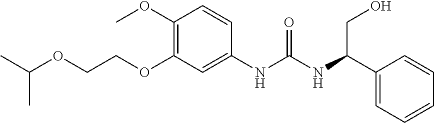 [SMILES: COc1ccc(NC(=O)N[C@@H](CO)c2ccccc2)cc1OCCOC(C)C] | 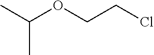 |
| SM0019 | 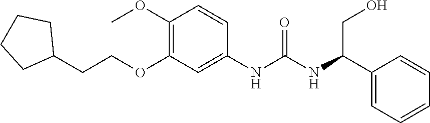 [SMILES: COc1ccc(NC(=O)N[C@@H](CO)c2ccccc2)cc1OCCC1CCCC1] | 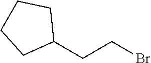 |
| SM0033 | 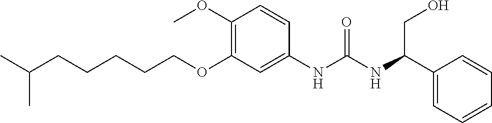 [SMILES: COc1ccc(NC(=O)N[C@@H](CO)c2ccccc2)cc1OCCCCCC(C)C] | 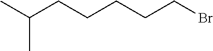 |
| SM0038 | 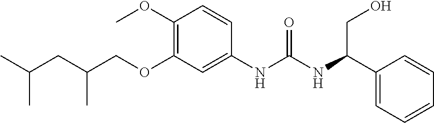 [SMILES: COc1ccc(NC(=O)N[C@@H](CO)c2ccccc2)cc1OCC(C)CC(C)C] | 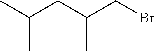 |
| SM0053 | 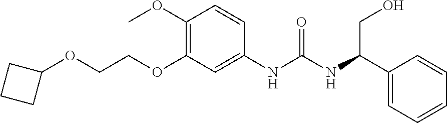 [SMILES: COc1ccc(NC(=O)N[C@@H](CO)c2ccccc2)cc1OCCOC1CCC1] | 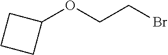 |
| SM0025 | 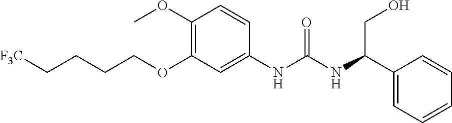 [SMILES: COc1ccc(NC(=O)N[C@@H](CO)c2ccccc2)cc1OCCCCC(F)(F)F] | 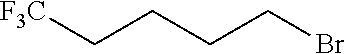 |

### SM0035 Synthesis and Compounds Synthesized by Similar Procedures

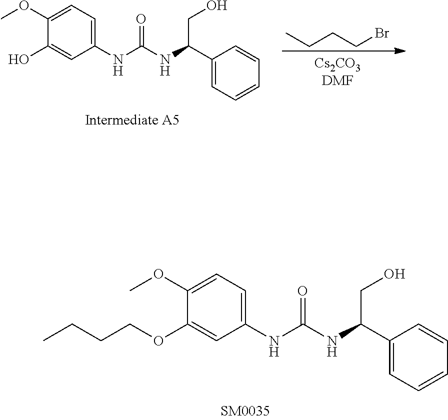

To a solution of Intermediate A5 (1-(3-hydroxy-4-methoxy-phenyl)-3-[(1R)-2-hydroxy-1-phenyl-ethyl]urea) (100 mg, 331 μmol, 1.00 eq) in dimethyl formamide (5 mL) is added cesium carbonate (216 mg, 662 μmol, 2.00 eq) and the alkyl halide dropwise. The mixture is stirred at 25° C. for 2 hours. The reaction mixture is poured into water (10 mL) and extracted with ethyl acetate. The combined organic layers are washed with brine, dried over sodium sulfate, filtered and concentrated under reduced pressure. The residue is purified by prep-HPLC and lyophilized to afford Compound SM0035 as a white solid.

The procedure is applied for synthesis of compounds listed in Table 16.

TABLE 16
Compounds synthesized by the procedure described above

| ID# | Structure | Alkyl halide |
|---|---|---|
| SM0072 | 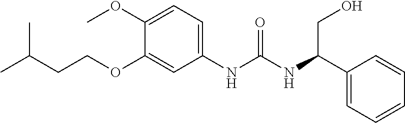 [SMILES: COc1ccc(NC(=O)N[C@@H](CO)c2ccccc2)cc1OCCC(C)C] | 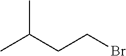 |
| SM0098 | 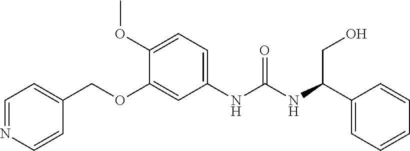 [SMILES: COc1ccc(NC(=O)N[C@@H](CO)c2ccccc2)cc1OCc1ccncc1] | 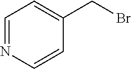 |
| SM0084 | 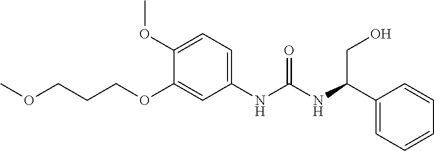 [SMILES: COCCCOc1cc(NC(=O)N[C@@H](CO)c2ccccc2)ccc1OC] | 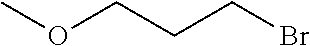 |
| SM0042 | 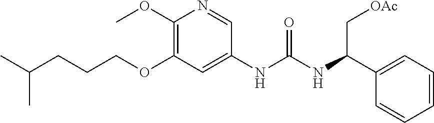 [SMILES: COc1ncc(NC(=O)N[C@@H](COC(C)=O)c2ccccc2)cc1OCCCC(C)C] | 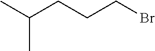 |

### SM0042 Synthesis

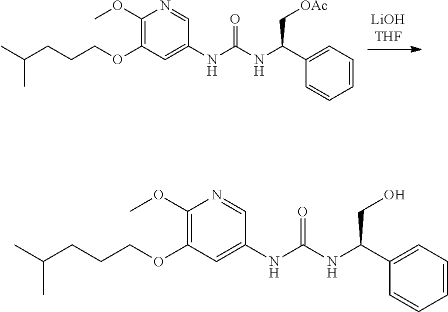 [SMILES: COc1ncc(NC(=O)N[C@@H](COC(C)=O)c2ccccc2)cc1OCCCC(C)C ; COc1ncc(NC(=O)N[C@@H](CO)c2ccccc2)cc1OCCCC(C)C]

To a solution of (R)-2-(3-(6-methoxy-5-((4-methylpentyl)oxy)pyridin-3-yl)ureido)-2-phenylethyl acetate (62 mg, 145 μmol, 1.0 eq). The solution is diluted with methanol (3 mL) and water (1 mL) is added lithium hydroxide mono hydrate (12.4 mg, 516 μmol, 5.00 eq). The mixture is stirred at 25° C. for 0.25 h, then diluted with water (20 mL) and extracted with ethyl acetate (20 mL×2). The combined organic layers are washed with brine, dried over sodium sulfate, filtered and concentrated under reduced pressure. The residue was purified by prep-HPLC and lyophilized to afford Compound SM0042 as a white solid.

### SM0051 Synthesis

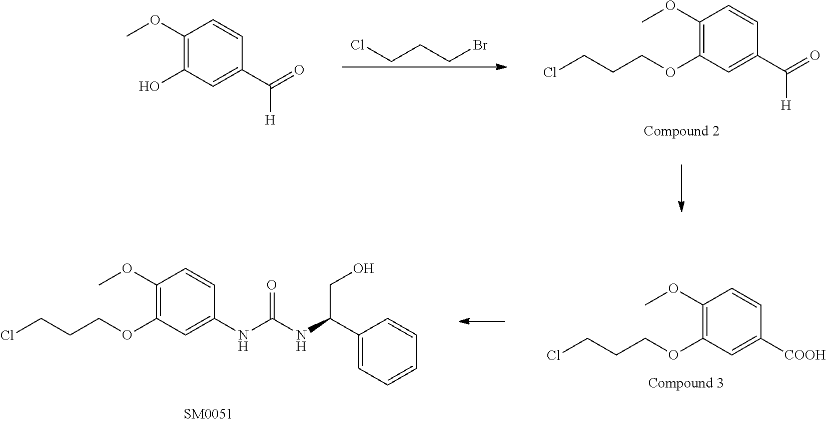 [SMILES: COc1ccc(C=O)cc1O ; ClCCCBr ; COc1ccc(C=O)cc1OCCCCl ; COc1ccc(NC(=O)N[C@@H](CO)c2ccccc2)cc1OCCCCl ; COc1ccc(C(=O)O)cc1OCCCCl]

Isovanillin is alkylated to make 3-(3-chloropropoxy)-4-methoxybenzaldehyde, which is then oxidized to the carboxylic acid, 3-(3-chloropropoxy)-4-methoxybenzoic acid. 3-(3-chloropropoxy)-4-methoxybenzoic acid is converted to the isocyanate and trapped with the amine, (R)-2-amino-2-phenylethan-1-ol, to make Compound SM0051.

### SM0060 Synthesis

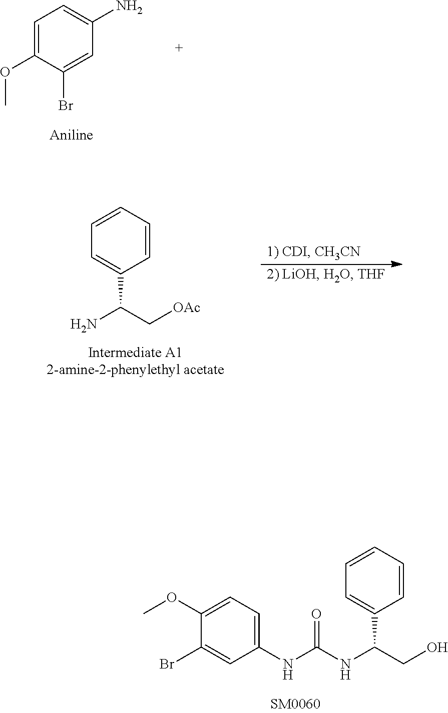

To a mixture of 3-bromo-4-methoxyaniline (aniline) (1.00 eq) in acetonitrile (3 mL) is added 1,1′-carbonyldiimidazole (1.0 eq). After stirred at 25° C. for 30 min, Intermediate A1 ((R)-2-amino-2-phenylethyl acetate) (1.0 eq) is added. The mixture is stirred at 25° C. for 4 h. The reaction mixture is concentrated under reduced pressure to give a residue. To a solution of residue (1.0 eq) in THF (2 mL) is added a solution of LiOH (5.0 eq) in water (1 mL). The mixture is stirred at 25° C. for 12 h. The reaction mixture is adjusted pH to 1 by 1M HCl and then extracted with ethyl acetate. The combined organic layers are washed with brine, dried over anhydrous sodium sulfate, filtered and concentrated under reduced pressure to give a residue. The residue is purified by prep-HPLC and lyophilized to give SM0060.

### SM0061 Synthesis

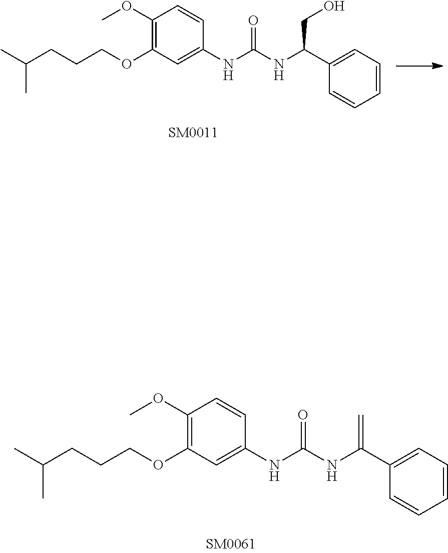 [SMILES: COc1ccc(NC(=O)N[C@@H](CO)c2ccccc2)cc1OCCCC(C)C ; C=C(NC(=O)Nc1ccc(OC)c(OCCCC(C)C)c1)c1ccccc1]

A solution of Compound SM0011 (1 eq, 150 mg, 0.38 mmol), phthalimide (2 eq, 114 mg, 0.77 mmol), and triphenylphosphine (1.8 eq, 183 mg, 0.7 mmol) in dry tetrahydrofuran (5 mL) is cooled in an ice bath. A solution of di-tert-butyl azodicarboxylate (1.5 eq, 134 mg, 0.77 mmol) in THF (500 μL) is then added slowly and dropwise. The reaction is slowly warmed to room temperature and then stirred for one hour. Reaction is then extracted into ethyl acetate and washed three times with water, dried over magnesium sulfate, and concentrated in vacuo. The crude oil is then dissolved into ethanol (100 mL) and hydrazine monohydrate (20 eq) was added, neat. The reaction is heated to reflux for 6 hours to yield the SM0061.

### SM0062 Synthesis

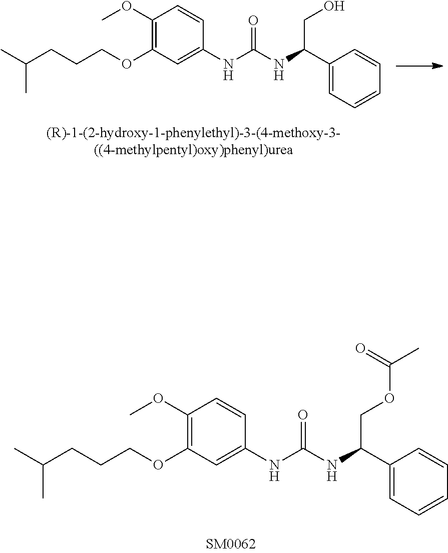 [SMILES: COc1ccc(NC(=O)N[C@@H](CO)c2ccccc2)cc1OCCCC(C)C ; COc1ccc(NC(=O)N[C@@H](COC(C)=O)c2ccccc2)cc1OCCCC(C)C]

To a solution of (R)-1-(2-hydroxy-1-phenylethyl)-3-(4-methoxy-3-((4-methylpentyl)oxy)phenyl)urea (0.13 mmol, 0.05 gm) in dichloromethane (5 mL) at 0° C. is added acetic anhydride (1.2 EQ.) followed by the slow addition of trimethylamine (1.2 EQ.). After 30 minutes the reaction is then quenched with water and extracted with DCM. The organics are washed with brine then dried over sodium sulfate, and filtered. The filtrate is concentrated under vacuum to give a crude oil. Purification of the oil is achieved using a C18 reverse phase column eluting with 0-100% acetonitrile in water over a 22 minute gradient. The desired fractions are combined and lyophilized to give the compound SM0062 as a white solid (31.9 mg).

### SM0068 Synthesis

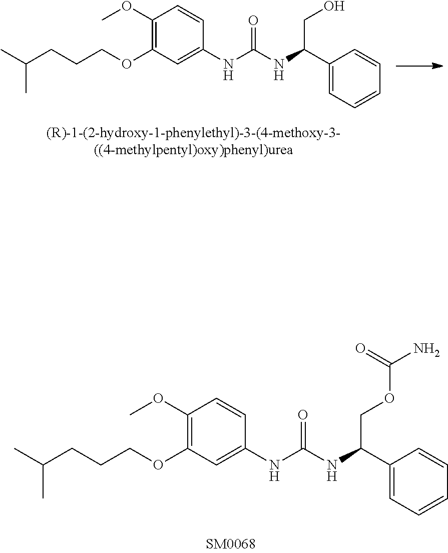 [SMILES: COc1ccc(NC(=O)N[C@@H](CO)c2ccccc2)cc1OCCCC(C)C ; COc1ccc(NC(=O)N[C@@H](COC(N)=O)c2ccccc2)cc1OCCCC(C)C]

A solution of (R)-1-(2-hydroxy-1-phenylethyl)-3-(4-methoxy-3-((4-methylpentyl)oxy)phenyl)urea (0.13 mmol, 0.05 gm) in dichloromethane (5 mL) at 0° C. is added triphosgene (44 mg). After a few minutes, ammonia (1M in THF) is added and the mixture warmed to room temperature. The mixture is then quenched with water and extracted with DCM. The organics are washed with brine then dried over sodium sulfate, and filtered. The filtrate is concentrated under vacuum to give a crude oil. Purification of the oil is achieved using a C18 reverse phase column eluting with 0-100% acetonitrile in water over a 22 minute gradient. The desired fractions are combined and lyophilized to give (R)-2-(3-(4-methoxy-3-((4-methylpentyl)oxyureido)-2-phenylethyl carbamate (14 mg) as a white solid.

### SM0069 Synthesis

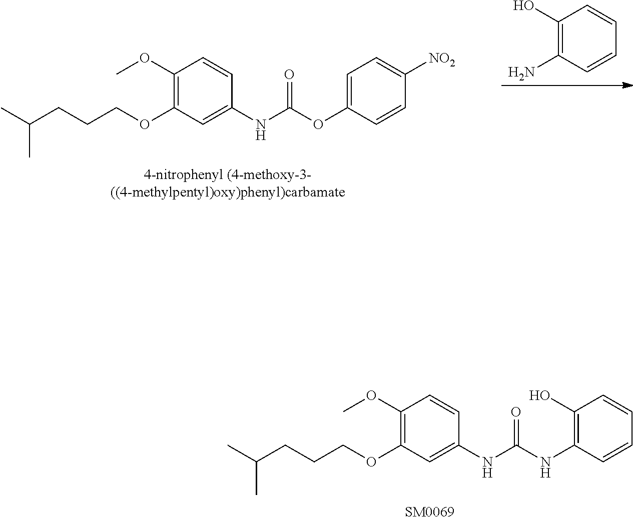 [SMILES: COc1ccc(NC(=O)Oc2ccc([N+](=O)[O-])cc2)cc1OCCCC(C)C ; Nc1ccccc1O ; COc1ccc(NC(=O)Nc2ccccc2O)cc1OCCCC(C)C]

To a solution of 4-nitrophenyl (4-methoxy-3-((4-methylpentyl) oxy)phenyl)carbamate (0.100 g, 0.256 mmol, 1 eq) in dry DMF (1 ml) is slowly added a solution of 2-hydroxy aniline (1 eq) and triethylamine (2 eq) in 1 mL DMF. The reaction is stirred at room temperature for 1.5 h. Upon completion, the solvent is removed and the residue is taken in EtOAc, washed the with 1N NaOH until aq layer becomes colorless, dried organic layer over anhydrous MgSO4 and concentrated. The crude is purified using reverse phase column chromatography using a gradient of 0-95% acetonitrile in water. The desired fractions are lyophilized to give the title compound as a white solid.

### SM0070 Synthesis

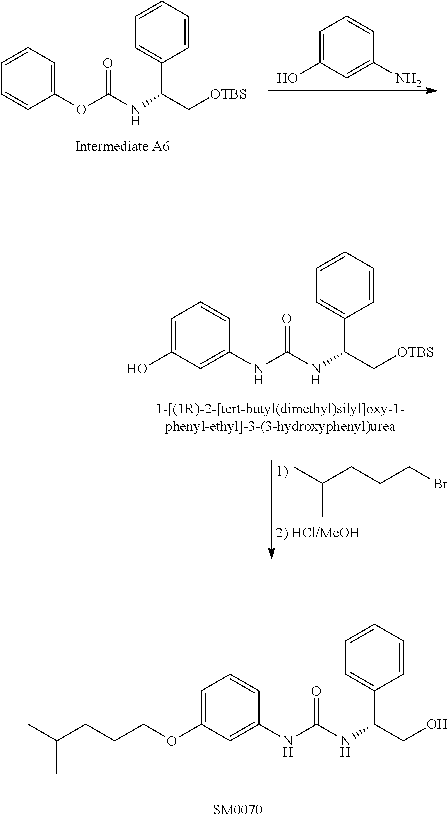

Step 1: To a mixture of Intermediate A6 (phenyl (R)-(2-((tert-butyldimethylsilyl)oxy)-1-phenylethyl)carbamate) (327 mg, 880 μmol, 1.20 eq) and triethylamine (223 mg, 2.20 mmol, 305 μL, 3.00 eq) in dioxane (3 mL) is added 3-aminophenol (80.0 mg, 733 μmol, 1.00 eq). The mixture is stirred at 110° C. for 12 hours. The reaction mixture is concentrated under reduced pressure to remove the solvent. The residue is diluted with water (10 mL) and extracted with ethyl acetate (10 mL×2). The combined organic layers are washed with water, dried over sodium sulfate, filtered and concentrated under reduced pressure to give a residue. The crude product 1-[(1R)-2-[tert-butyl(dimethyl)silyl]oxy-1-phenyl-ethyl]-3-(3-hydroxyphenyl)urea (200 mg) is used into the next step without further purification.

Steps 2 and 3: To a mixture of 1-[(1R)-2-[tert-butyl(dimethyl)silyl]oxy-1-phenyl-ethyl]-3-(3-hydroxyphenyl)urea (200 mg, 517 μmol, 1.00 eq) and cesium carbonate (337 mg, 1.03 mmol, 2.00 eq) in dimethyl formamide (2 mL) is added 1-bromo-4-methyl-pentane (102 mg, 620 μmol, 87.6 μL, 1.20 eq). The mixture is stirred at 25° C. for 12 hours. The reaction mixture is diluted with water (20 mL) and extracted with ethyl acetate (10 mL×2). The combined organic layers are washed with water, dried over sodium sulfate, filtered and concentrated under reduced pressure to give a residue.

The crude product is dissolved in methanol (2 mL) and added hydrochloric acid/methanol (4 M, 531 μL) is added. The mixture is stirred at 25° C. for 2 hours. The reaction mixture is concentrated under reduced pressure to remove solvent. The mixture is further purified by pre-HPLC and lyophilized to give 1-[(1R)-2-hydroxy-1-phenyl-ethyl]-3-(3-isohexyloxyphenyl)urea (66.7 mg, 187 μmol, 88% yield, 100% purity) as a white solid.

### SM0037 Synthesis

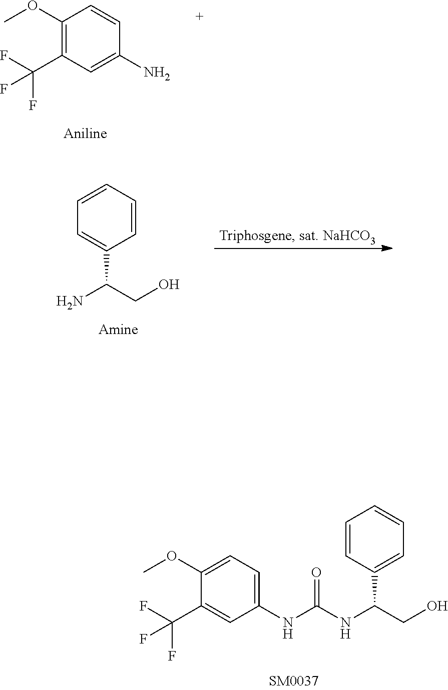 [SMILES: COc1ccc(N)cc1C(F)(F)F ; N[C@@H](CO)c1ccccc1 ; "Triphosgene, sat. NaHCO3" ; COc1ccc(NC(=O)N[C@@H](CO)c2ccccc2)cc1C(F)(F)F]

To a solution of 4-methoxy-3-(trifluoromethyl)aniline (aniline), (1.00 eq) in dichloromethane (3 ml) is added triphosgene (0.35 eq) at 0° C. Then saturated sodium bicarbonate (3 mL) is added dropwise to the mixture at 0° C. After stirred for 1 h, the mixture is allowed to stand for 5 min. The organic layer is separated, dried over anhydrous sodium sulfate and filtered. (R)-2-amino-2-phenylethan-1-ol (amine) (1.00 eq) is added into organic layer at 0° C. The mixture is stirred at 0-25° C. for another 4 h. After removal of solvent, the residue is purified by prep-HPLC and lyophilized to give target molecule.

### SM0075 Synthesis

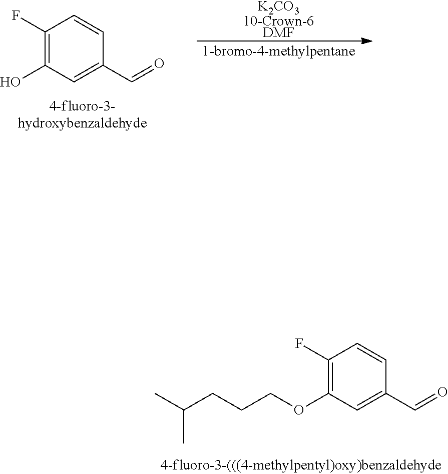 [SMILES: O=Cc1ccc(F)c(O)c1 ; "1-bromo-4-methylpentane" ; "K2CO310-Crown-6DMF" ; CC(C)CCCOc1cc(C=O)ccc1F]

Step 1: To a mixture of 4-fluoro-3-hydroxybenzaldehyde (5.7 mmol, 0.8 g), potassium carbonate (8.6 mmol, 1.2 g), and 18-Crown-6 (1.4 g) in DMF (10 mL) is added 1-bromo-4-methylpentane (6.8 mmol, 1.1 g). The resulting mixture is stirred for 12 hours then quenched with water and extracted with ethyl acetate. The organics are washed with brine then dried over sodium sulfate, and filtered. The filtrate is concentrated under vacuum to give a crude oil. Purification of the oil is achieved by chromatography on silica using 40% ethyl acetate in n-hexane as eluant to give 4-fluoro-3-(((4-methylpentyl)oxy)benzaldehyde (1.01 g) as a clear oil.

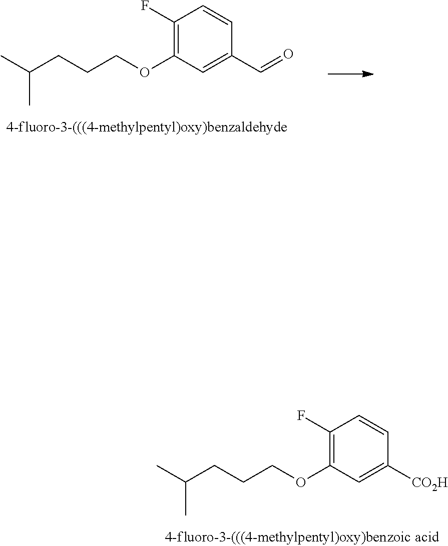 [SMILES: CC(C)CCCOc1cc(C=O)ccc1F ; CC(C)CCCOc1cc(C(=O)O)ccc1F]

Step 2: To a solution of 4-fluoro-3-(((4-methylpentyl)oxy)benzaldehyde (4.5 mmol, 1.0 g) in acetonitrile (10 mL) at 0° C. is added 30% hydrogen peroxide solution (6.7 mmol, 0.3 mL), a solution of sodium phosphate monobasic hydrate (0.02 g) and sodium chlorite (6.7 mmol, 0.60 g) in water (2 mL). The resulting solution is warmed to room temperature and allowed to stir for an addition 24 hours. The reaction is quenched with a saturated solution of sodium thiosulfate then extracted with ethyl acetate. The organics are washed with brine then dried over sodium sulfate, and filtered. The filtrate is concentrated under vacuum to give a crude oil. Purification of the oil is achieved by chromatography on silica using 40% ethyl acetate in n-hexane as eluant to give 4-fluoro-3-(((4-methylpentyl)oxy)benzoic acid (0.9 g) as a white solid.

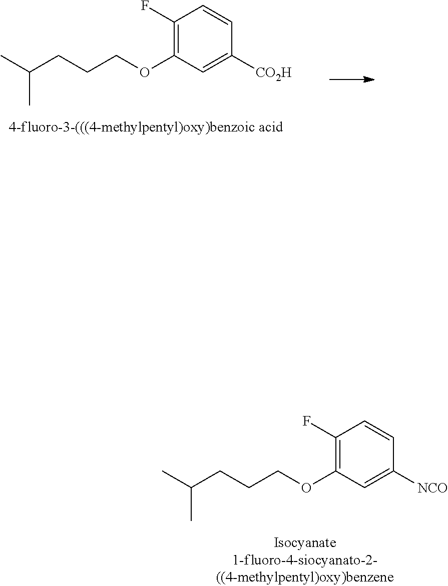

Step 3: A mixture of 4-fluoro-3-(((4-methylpentyl)oxy)benzoic acid (3.7 mmol, 0.9 g), diphenyl phosphoryl azide (4.5 mmol, 1.2 g), and triethylamine (5.3 mmol, 0.548 g) in toluene (20 mL) is heated at 70° C. for 12 hours. The mixture is cooled and concentrated under vacuum to give a 1-fluoro-4-isocyanato-2-((4-methylpenyl)oxy)benzene as a crude oil.

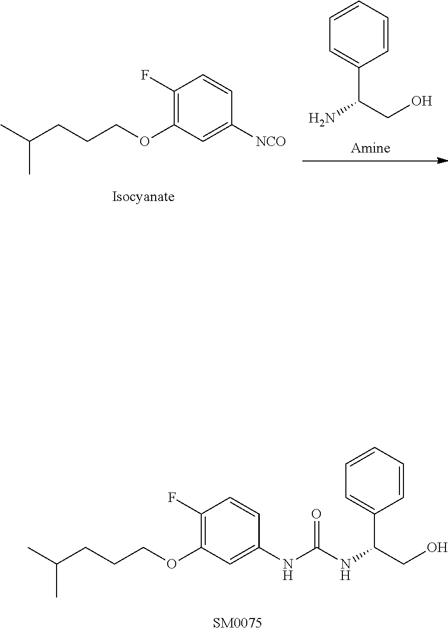

Step 4: To a solution of the isocyanate of Step 3 in DCM (1 mL) is added a solution of (R)-2-amino-2-phenylethan-1ol (an amine, 2-amino-2-(4-fluorophenyl)ethan-1-ol, (CAS #140373-17-7) (1 eq) in DCM (1 mL). The reaction is stirred for 15 minutes, volatiles were removed in vacuo. The compound is purified on C18 reverse phase flash chromatography 5-95% B MeCN.

### SM0096 Synthesis

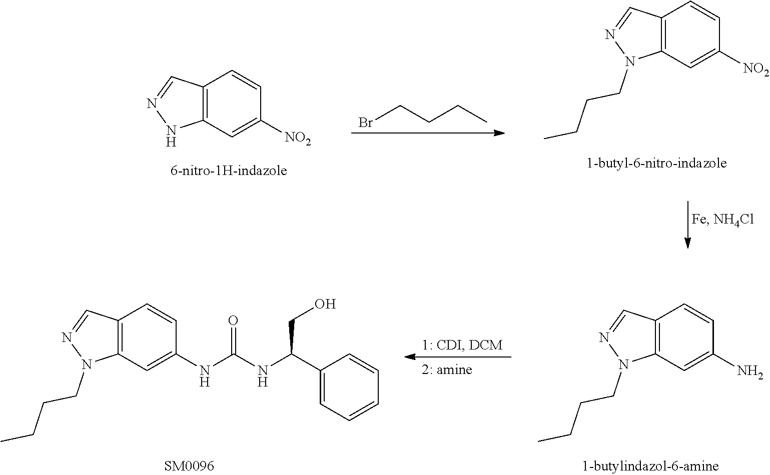

Step 1: To a mixture of 6-nitro-1H-indazole (1.00 g, 6.13 mmol, 1.00 eq) in DMF (20 mL) is added NaH (221 mg, 9.20 mmol, 1.50 eq) at 0′° C. After stirred for 30 min, 1-bromobutane (1.01 g, 7.36 mmol, 794 μL, 1.20 eq) is added. The mixture is stirred at 25° C. for 12 hr. The mixture is quenched by water (20 mL), extracted with ethyl acetate (50 mL*2). The combined organic phase is washed with brine (50 mL*2), dried over anhydrous Na₂SO₄, filtered and concentrated in vacuum. The residue is purified by silica gel chromatography (petroleum ether/ethyl acetate=8/1 to 3/1) to give 1-butyl-6-nitro-indazole (400.00 mg, 1.82 mmol, 30% yield) as a yellow solid.

Step 2: To a solution of 1-butyl-6-nitro-indazole (350 mg, 1.60 mmol, 1.00 eq) in H₂O (10.00 mL) and EtOH (30 mL) is added NH₄Cl (856 mg, 16.0 mmol, 559 μL, 10.0 eq). The solution is stirred for 30 min at 80° C. Then Fe (446.80 mg, 8.00 mmol, 5.00 eq) is added in one portion. The mixture is stirred at 80° C. for 4 hrs. The solid is removed by filtration. The filtrate is extracted with ethyl acetate (30 mL*2). The combined organic phase is washed with brine, dried over anhydrous Na₂SO₄, filtered and concentrated in vacuum. The crude product 1-butylindazol-6-amine is used into the next step without further purification.

Step 3: To a solution of 1-butylindazol-6-amine (300 mg, 1.59 mmol, 1.00 eq) in DCM (5.00 mL) is added CDI (284 mg, 1.75 mmol, 1.10 eq). After stirred for 1 hr, the amine (2R)-2-amino-2-phenyl-ethanol (261.74 mg, 1.91 mmol, 1.20 eq) is added. The mixture is stirred at 25° C. for 5 hrs. The mixture is concentrated in reduced pressure. The residue is purified by prep-HPLC and lyophilized to give 1-(1-butylindazol-6-yl)-3-[(1R)-2-hydroxy-1-phenyl-ethyl]urea (30.86 mg, 77.30 μmol, 4.86% yield, 99.8% purity, FA) as a white solid.

### SM0101 Synthesis

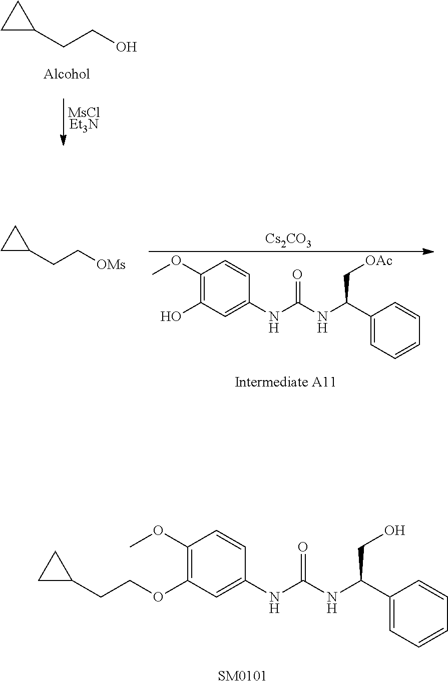

Step 1: To a solution of 2-cyclopropylethan-1-ol (alcohol) (1.00 eq) in dichloromethane (5 mL) is added triethylamine (1.34 g, 13.2 mmol, 1.83 mL, 1.50 eq) and methanesulfonyl chloride (1.21 g, 10.6 mmol, 818 μL, 1.20 eq) dropwise at 0° C. The mixture is stirred at 25° C. for 1 hour. The reaction mixture is diluted with water (10 mL) and extracted with dichloromethane (3 mL×3). The combined organic layers are washed with brine, dried over sodium sulfate, filtered and concentrated under reduced pressure to afford 2-cyclopropylethyl methanesulfonate.

Step 2: To a solution of Intermediate A11 ([(2R)-2-[(3-hydroxy-4-methoxy-phenyl)carbamoylamino]-2-phenyl-ethyl] acetate) (1.50 g, 4.36 mmol, 1.00 eq) in dimethyl formamide (20 mL) is added cesium carbonate (5.68 g, 17.42 mmol, 4.00 eq) and the product from Step 1 (1.50 eq). The mixture is stirred at 85° C. for 12 hours. The reaction mixture is poured into water (50 mL) at 0° C., extracted with ethyl acetate. The combined organic layers are washed with brine, dried over sodium sulfate, filtered and concentrated under reduced pressure to give a residue. The residue is purified by prep-HPLC and lyophilized to afford SM0101.

### SM0105 and SM0115 Synthesis

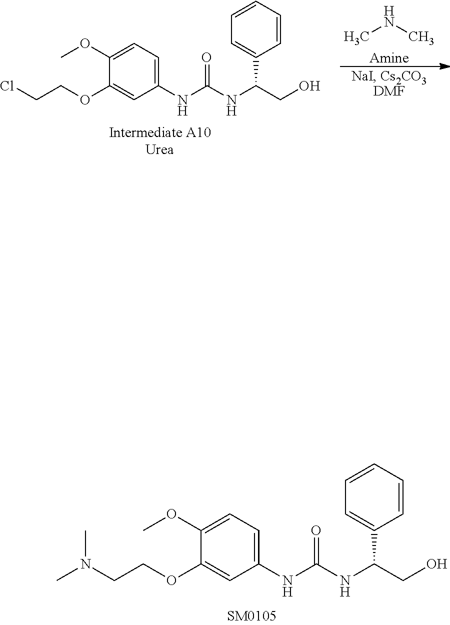

To a mixture of Intermediate A10 (1-[3-(2-chloroethoxy)-4-methoxy-phenyl]-3-[(1R)-2-hydroxy-1-phenyl-ethyl]urea) (100 mg, 274.1 μmol, 1.0 eq) and sodium iodide (49.3 mg, 329 μmol, 1.2 eq), cesium carbonate (179 mg, 548 μmol, 2.0 eq) in dimethylformamide (1 mL) is added N, N′-dimethylamine (amine, 12.4 mg, 274 μmol, 13.9 μL, 1.0 eq). The mixture is stirred at 100° C. in microwave reactor for 1 hour. The reaction mixture is diluted with water (20 mL) and extracted with ethyl acetate (20 mL×2). The combined organic layers are washed with water, dried over sodium sulfate, filtered and concentrated under reduced pressure. The residue is purified by prep-HPLC, the solution of ACN and lyophilized to afford 1-[3-[2-(dimethylamino)ethoxy]-4-methoxy-phenyl]-3-[(1R)-2-hydroxy-1-phenyl-ethyl]urea (22.53 mg, 60.3 μmol, 22% yield, 100% purity) as brown oil.

The procedure is applied for synthesis of SM0115, as shown in Table 17.

TABLE 17
Compounds synthesized by the procedure described above

| | | Inter- | |
|---|---|---|---|
| | | mediate | |
| ID# | Structure | (Urea) | (Amine) |
| SM0115 | 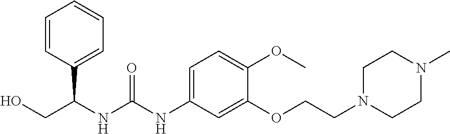 [SMILES: COc1ccc(NC(=O)N[C@@H](CO)c2ccccc2)cc1OCCN1CCN(C)CC1] | A12 | 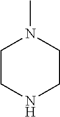 |

### SM0107 Synthesis

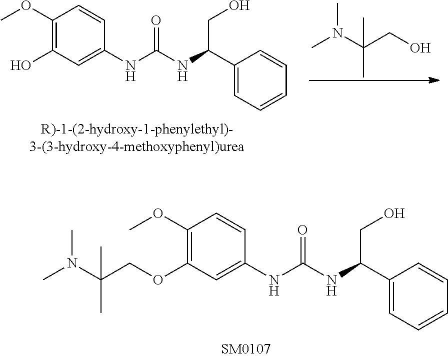

(R)-1-(2-hydroxy-1-phenylethyl)-3-(3-hydroxy-4-methoxyphenyl)urea (100 mg, 0.35 mmol) is treated with DIAD (1.2 EQ) Triphenyl phosphine (1.2 EQ.) and then the primary alcohol 2-(dimethylamino)-2-methylpropan-1-ol (1.2 EQ.) The reaction is stirred at room temperature overnight. The reaction is quenched by the addition of saturated aqueous ammonium chloride. The reaction mixture is extracted with ethyl acetate and the organics were further washed with brine. The combined organics are dried over MgSO4, filtered and concentrated in vacuo to provide the desired product. The compound is purified by C-18 reverse phase chromatography using a 0-100% ACN (0.1% TFA) in water gradient over 15 minutes. The desired fractions are concentrated to provide the desired product (78 mg).

### SM0110 Synthesis

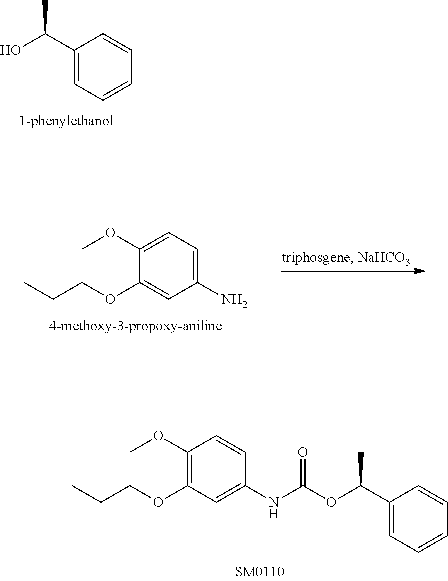

To a solution of 4-methoxy-3-propoxy-aniline (100 mg, 459 μmol, 1.0 eq, HCl) in dichloromethane (3 mL) is added triphosgene (47.7 mg, 161 μmol, 0.35 eq). Then saturated sodium bicarbonate (3 mL) is added dropwise to the mixture. After stirred for 1 h, the organic layer is separated, dried over anhydrous sodium sulfate and filtered. (1S)-1-phenylethanol (56.1 mg, 459 μmol, 55.6 μL, 1.00 eq) is added into organic layer. The mixture is stirred at 25° C. for another 4 h. The reaction mixture is concentrated under reduced pressure to give a residue. The residue is purified by prep-HPLC to give (S)-2-(4-methoxy-3-propoxyphenyl)-N-(1-phenylethyl)acetamide (39.73 mg, 121 μmol, 26.3% yield, 100% purity) as a yellow solid.

### SM0114 Synthesis

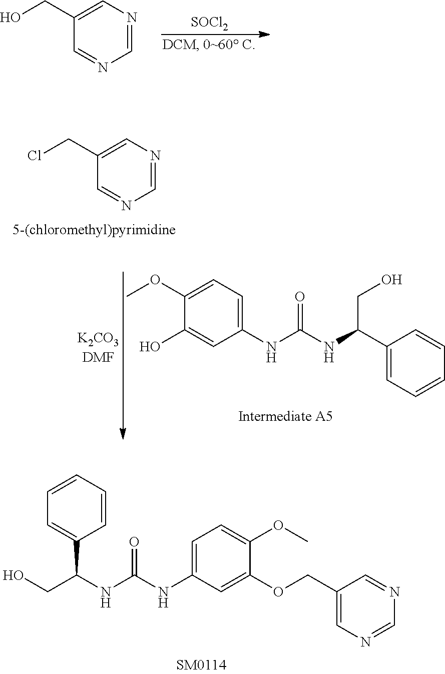

Step 1: To a solution of pyrimidin-5-ylmethanol (100 mg, 908 μmol, 1.0 eq) in dichloromethane (10 mL) is added thionyl chloride (864 mg, 7.27 mmol, 527 μL, 8.0 eq) dropwise at 0° C., then the mixture is stirred at 50° C. for 2 hours. The reaction mixture is concentrated under reduced pressure to give 5-(chloromethyl)pyrimidine (100 mg, crude) as yellow oil.

Step 2: To a solution of Intermediate A5 (1-(3-hydroxy-4-methoxy-phenyl)-3-[(1R)-2-hydroxy-1-phenyl-ethyl]urea) (100 mg, 331 μmol, 1.0 eq) in dimethyl formamide (5 mL) is added cesium carbonate (216 mg, 662 μmol, 2.0 eq) and 5-(chloromethyl)pyrimidine (63.8 mg, 496 μmol, 1.5 eq). The mixture is stirred at 25° C. for 12 hrs. The reaction mixture is poured into water, and then extracted with dichloromethane:isopropanol. The combined organic layers are dried over sodium sulfate, filtered and concentrated under reduced pressure. The residue is purified by prep-HPLC and lyophilized to afford 1-[(1R)-2-hydroxy-1-phenyl-ethyl]-3-[4-methoxy-3-(pyrimidin-5-ylmethoxy) phenyl] urea (54.3 mg, 138 μmol, 41.6% yield, 100% purity) as a white solid.

### SM0116 Synthesis

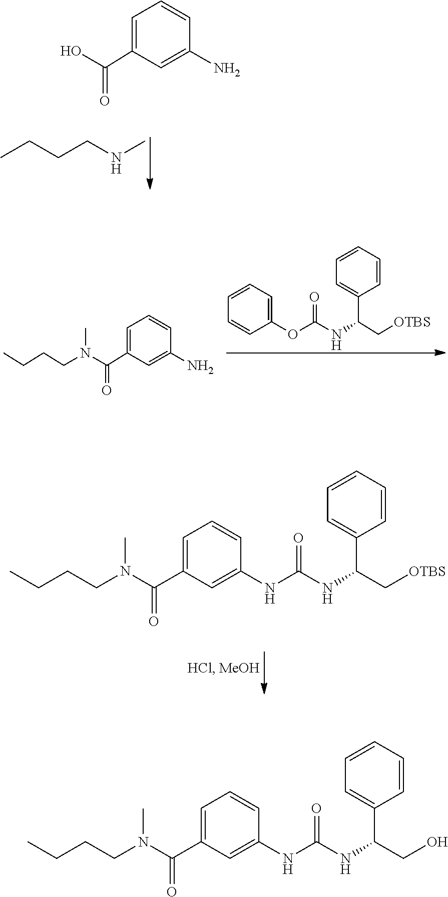

Step 1: To a mixture of 3-aminobenzoic acid (200 mg, 1.46 mmol, 1.00 eq) and propylphosphonic anhydride (T3P, 697 mg, 2.19 mmol, 651 μL, 1.50 eq) in acetonitrile (3 mL) is added triethylamine (295 mg, 2.92 mmol, 405 μL, 2.00 eq), N-methylbutan-1-amine (153 mg, 1.75 mmol, 206 μL, 1.20 eq). The mixture is stirred at 25° C. for 12 hours. The reaction mixture is concentrated. The residue is diluted with water (20 mL) and extracted with ethyl acetate (10 mL×2). The combined organic layers are washed with water, dried over sodium sulfate, filtered and concentrated under reduced pressure to give a residue. The crude product 3-amino-N-butyl-N-methyl-benzamide (200 mg) is used into the next step without further purification.

Step 2: To a mixture of 3-amino-N-butyl-N-methyl-benzamide (200 mg, 970 μmol, 1.00 eq) and triethylamine (196 mg, 1.94 mmol, 269 μL, 2.00 eq) in dioxane (3 mL) is added phenyl N-[(1R)-2-[tert-butyl(dimethyl)silyl] oxy-1-phenyl-ethyl]carbamate (432 mg, 1.16 mmol, 1.20 eq). The mixture is stirred at 110° C. for 12 hours. The reaction mixture is concentrated to give a residue. The residue is diluted with water (20 mL) and extracted with ethyl acetate (10 mL×2). The combined organic layers are washed with water, dried over sodium sulfate, filtered and concentrated under reduced pressure to give a residue. The crude product N-butyl-3-[[(1R)-2-[tert-butyl(dimethyl)silyl]oxy-1-phenyl-ethyl]carbamoylamino]-N-methyl-benzamide (100 mg) is used into the next step without further purification.

Step 3: To a mixture of N-butyl-3-[[(1R)-2-[tert-butyl(dimethyl)silyl]oxy-1-phenyl-ethyl]carbamoylamino]-N-methyl-benzamide (100 mg, 207 μmol, 1.00 eq) in methanol (2 mL) is added HCl solution (4 M in methanol, 2 mL). The mixture is stirred at 25° C. for 2 hours. The mixture is concentrated in vacuum. The reaction mixture is concentrated and the residue is diluted with water (20 mL). The mixture is extracted with ethyl acetate (10 mL×2). The combined organic layers are washed with water, dried over sodium sulfate, filtered and concentrated under reduced pressure to give a residue. The residue is purified by pre-HPLC and lyophilized to give N-butyl-3-[[(1R)-2-hydroxy-1-phenyl-ethyl]carbamoylamino]-N-methyl-benzamide (58.47 mg, 140 μmol, 68% yield, 100% purity) as a brown oil.

### SM0119 Synthesis

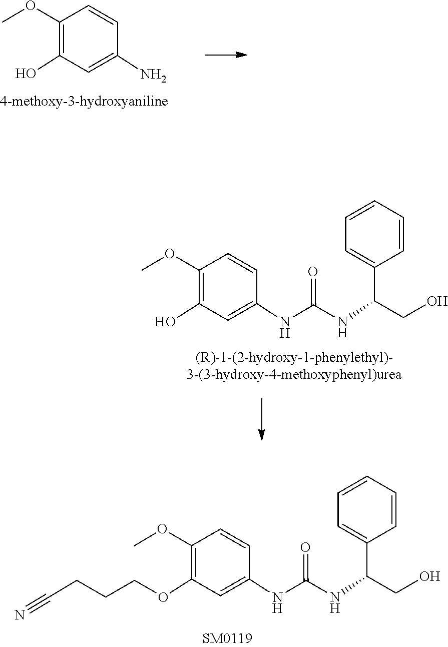

Step 1: To a solution of p-nitrophenylchloroformate (0.300 g, 1.5 mmol, 1 eq) in dry DCM (4 mL) is added solid 4-methoxy-3-hydroxyaniline (0.189 g, 1.5 mmol, 1 eq) slowly portion wise. The reaction is stirred at room temperature for 15 mins and the solvent is stripped off. The residue is taken in dry DMF (2 mL) and a solution of (R)-2-amino-2-phenylethan-1-ol (0.206 g, 1.5 mmol, 1 eq) and TEA (0.303 g, 0.42 mL, 3 mmol, 2 eq) in 4 mL DMF is added slowly. The reaction is stirred at room temperature for 1 h. Upon completion, the solvent is removed and the residue was taken in EtOAc, washed with water, dried the organic layer over anhydrous MgSO₄ and concentrated. Purified crude by normal phase chromatography using a gradient of 0-100% EtOAc in hexanes to give (R)-1-(2-hydroxy-1-phenylethyl)-3-(3-hydroxy-4-methoxyphenyl)urea as a tan solid (0.152 g, 34%).

Step 2: To a solution of (R)-1-(2-hydroxy-1-phenylethyl)-3-(3-hydroxy-4-methoxyphenyl)urea (0.152 g, 0.503 mmol, 1 eq) in DMF (5 mL) is added K2CO3 (0.139 g, 1.006 mmol, 2 eq), 4-chlorobutanenitrile (0.155 g, 1.509 mmol, 3 eq), 18-crown-6 (0.013 g, 0.0503 mmol, 0.1 eq), NaI (0.226 g, 1.509 mmol, 3 eq) at room temperature. The reaction is stirred at 80° C. overnight. Upon completion, the reaction is quenched with 20 mL water, extracted with EtOAc (50 ml×2), dried EtOAc layer over anhydrous MgSO4 and concentrated. The crude is purified using reverse phase column chromatography using a gradient of 0-95% acetonitrile in water. The desired fractions are lyophilized to give the title compound as a white solid (0.024 g, 13%).

### Example 9. Compound Analysis by Surface Plasmon Resonance (SPR)

Surface plasmon resonance (SPR) technology was used to generate data on the affinity, specificity, and kinetics of complement C5 and inhibitor interactions in real time without the need for labeling.

SensiQ FE SPR system (SensiQ Technologies, Oklahoma City, Okla.) was used to provide sensitive and accurate detection of binding of small molecules to the very large C5 protein (MW=195,000 Da). The chip was prepared by preconditioning the sensor according to the protocol of the SensiQ FE using 10 mM HCl, 50 mM NAOH and 0.1% SDS. The sensor chip was activated by using a mixture of fresh EDAC (1-ethyl-3-(-3-dimethylaminopropyl) carbodiimide) (Sigma Co., St. Louis, Mo.) and NHS (N-hydroxy succinimide) (Sigma Co., St. Louis, Mo.). Human C5 was surface immobilized to a Pioneer Biosensor chip via random amine coupling (>12,000 RU) which makes use of the N-terminus and ε-amino groups of lysine residues of the protein ligand. Immobilization was done by injecting 30-40 ug/ml C5 in 10 mM NaAc pH 4.5 onto designated channels at a rate of 10 μL/minute for 12 minutes, targeting RL>12000 RU for small molecules.

C5 binding compounds, indicated in Table 1, were synthesized according to standard methods known in the art [see, e.g. Morrison and Boyd in “Organic Chemistry”, 6^{th} edition, Prentice Hall (1992)] and validated for proper structure by mass spectrometry. Compounds were diluted in DMSO in a format of 100-fold final concentration and 3-fold serial dilution (5 or 6 dilutions). The 100-fold compounds were transferred to one fold DMSO-free assay buffer in the 96-well test plate. The compound solution was injected at a rate of 60 μL/minute for 30-60 seconds, followed by 60-90 seconds dissociation time, buffer flushing and/or priming. Blank solution (1% DMSO assay buffer) was run for every 6 injections of compounds. Double reference by subtracting both blank channel and reference channel was applied for data processing. Titration of C5 binding compounds to the C5-immobilized biosensor chip surface led to interactions between C5 and potential binders, and the resulting changes of surface refractive index were sensitively measured by the system.

SPR data were analyzed with the managing software provided by SensiQ and equilibrium dissociation constant (K_{D}) values were determined for each compound. Values obtained are presented in Table 18. Compounds C5INH-0395, C5INH-0519, C5INH-0348, C5INH-0517, C5INH-0518, C5INH-0516, and C5INH-0521 had an equilibrium dissociation constant for interaction with C5 of less than 20 nM.

TABLE 18
Surface plasmon resonance (SPR) data

| | SPR C5 EQ |
|---|---|
| Compound ID | K_{D} (nM) |
| C5INH-0395 | 9.0 |
| C5INH-0519 | 9.3 |
| C5INH-0348 | 10.2 |
| C5INH-0517 | 10.7 |
| C5INH-0518 | 14.0 |
| C5INH-0516 | 16.0 |
| C5INH-0521 | 16.0 |
| C5INH-0311 | 20.0 |
| C5INH-0296 | 20.0 |
| C5INH-0486 | 25.0 |
| C5INH-0453 | 25.0 |
| C5INH-0526 | 27.0 |
| C5INH-0524 | 27.0 |
| C5INH-0456 | 29.0 |
| C5INH-0545 | 31.0 |
| C5INH-0536 | 32.0 |
| C5INH-0525 | 34.0 |
| C5INH-0437 | 37.0 |
| C5INH-0391 | 37.0 |
| C5INH-0476 | 40.0 |
| C5INH-0310 | 40.0 |
| C5INH-0547 | 42.0 |
| C5INH-0377 | 47.0 |
| C5INH-0401 | 48.0 |
| C5INH-0287 | 49.0 |
| C5INH-0315 | 50.0 |
| C5INH-0339 | 52.0 |
| C5INH-0361 | 52.0 |
| C5INH-0316 | 58.0 |
| C5INH-0452 | 59.0 |
| C5INH-0356 | 59.0 |
| C5INH-0491 | 62.0 |
| C5INH-0473 | 69.0 |
| C5INH-0340 | 75.0 |
| C5INH-0538 | 79.0 |
| C5INH-0537 | 81.0 |
| C5INH-0472 | 82.0 |
| C5INH-0366 | 82.0 |
| C5INH-0398 | 89.0 |
| C5INH-0462 | 95.0 |
| C5INH-0333 | 98.0 |
| C5INH-0488 | 100.0 |
| C5INH-0383 | 102.0 |
| C5INH-0367 | 102.0 |
| C5INH-0369 | 104.0 |
| C5INH-0409 | 110.0 |
| C5INH-0381 | 120.0 |
| C5INH-0357 | 135.0 |
| C5INH-0350 | 160.0 |
| C5INH-0336 | 164.0 |
| C5INH-0342 | 180.0 |
| C5INH-0335 | 180.0 |
| C5INH-0382 | 181.0 |
| C5INH-0446 | 184.0 |
| C5INH-0317 | 190.0 |
| C5INH-0385 | 190.0 |
| C5INH-0338 | 195.0 |
| C5INH-0474 | 201.0 |
| C5INH-0447 | 204.0 |
| C5INH-0544 | 210.0 |
| C5INH-0403 | 220.0 |
| C5INH-0458 | 225.0 |
| C5INH-0390 | 257.0 |
| C5INH-0326 | 260.0 |
| C5INH-0535 | 260.0 |
| C5INH-0534 | 260.0 |
| C5INH-0402 | 261.0 |
| C5INH-0490 | 278.0 |
| C5INH-0463 | 280.0 |
| C5INH-0454 | 284.0 |
| C5INH-0373 | 293.0 |
| C5INH-0443 | 300.0 |
| C5INH-0318 | 313.0 |
| C5INH-0504 | 316.0 |
| C5INH-0387 | 320.0 |
| C5INH-0485 | 330.0 |
| C5INH-0501 | 330.0 |
| C5INH-0500 | 334.0 |
| C5INH-0489 | 364.0 |
| C5INH-0425 | 410.0 |
| C5INH-0389 | 420.0 |
| C5INH-0539 | 430.0 |
| C5INH-0355 | 441.0 |
| C5INH-0379 | 499.0 |
| C5INH-0414 | 500.0 |
| C5INH-0436 | 510.0 |
| C5INH-0432 | 530.0 |
| C5INH-0428 | 530.0 |
| C5INH-0298 | 550.0 |
| C5INH-0460 | 578.0 |
| C5INH-0353 | 580.0 |
| C5INH-0496 | 600.0 |
| C5INH-0469 | 600.0 |
| C5INH-0527 | 650.0 |
| C5INH-0372 | 670.0 |
| C5INH-0417 | 693.0 |
| C5INH-0352 | 700.0 |
| C5INH-0303 | 710.0 |
| C5INH-0323 | 731.0 |
| C5INH-0406 | 760.0 |
| C5INH-0484 | 770.0 |
| C5INH-0420 | 800.0 |
| C5INH-0343 | 800.0 |
| C5INH-0384 | 810.0 |
| C5INH-0410 | 840.0 |
| C5INH-0450 | 885.0 |
| C5INH-0388 | 970.0 |
| C5INH-0477 | 1130.0 |
| C5INH-0431 | 1160.0 |
| C5INH-0349 | 1210.0 |
| C5INH-0422 | 1340.0 |
| C5INH-0497 | 1400.0 |
| C5INH-0396 | 1400.0 |
| C5INH-0498 | 1480.0 |
| C5INH-0421 | 1660.0 |
| C5INH-0502 | 1910.0 |
| C5INH-0397 | 2060.0 |
| C5INH-0492 | 2070.0 |
| C5INH-0438 | 2780.0 |
| C5INH-0330 | 2900.0 |
| C5INH-0329 | 3200.0 |
| C5INH-0440 | 7600.0 |

### Example 10. Compound Analysis by Red Blood Cell (RBC) Hemolysis Assay

Experiments were carried out using an RBC hemolysis assay to assess the ability of each compound to inhibit the lysis of RBCs. This assay identifies compounds capable of reducing lysis of sheep erythrocytes resulting from terminal complex formation. The assay was carried out using 1.5% human C5 depleted sera and 0.5 nM purified human C5.

GVB++ buffer was heated at 37° C. for a minimum of 20 minutes. The human C5 depleted sera and purified human C5 were rapidly thawed at 37° C. and then stored on ice or wet ice, respectively. The compound stock (10 mM, DMSO) was serially diluted in 100% DMSO to obtain 10 6-fold dilutions before addition of GVB++. Sera dilution was prepared by adding 5 mL of GVB++ to a 15 mL conical tube, removing 600 μL of the GVB++ and adding 600 μL of the 100% sera. The tube was mixed by inverting three times. A volume of 25 μL of the diluted sera was added to each well so that the final concentration of sera in the well was 1.5%. C5 dilution was prepared by adding 5 mL of GVB++ to a 15 mL conical tube, removing 4 μL of the GVB++ and adding 4 μL of the C5 stock. The tube was mixed by inverting three times. A volume of 25 μL was added to each well so that the final amount of C5 was 0.5 nM in each well. The antibody-sensitized sheep erythrocytes (EAs) were centrifuged at 1,000× gravity at 22° C. for 3 minutes. The supernatant was pipetted off without disrupting the pellet. The pellet was then resuspended to GVB++(Complement Technology, Tyler, Tex.) with the same volume as was removed. The resuspended EAs were mixed by gently inverting the tube.

Five controls were run on each plate: (1) EAs only=I00 μL EAs+50 μL GVB++ with 4% DMSO+50μL GVB++; (2) EA+Sera=100 μL EAs+50 μL GVB++ with 4% DMSO+25 μL Sera dilution+25 μL GVB++; (3) EA+C5=100 μL EAs+50 μL GVB++ with 4% DMSO+25 μL C5 dilution+25 μL GVB++; (4) EA+Sera+C5=100 μL EAs+50 μL GVB++ with 4% DMSO+25 μL Sera dilution+25 μL C5; (5) GVB++ Only=200 μL GVB++. Other wells included: GVB++ with 4% DMSO=20 μL DMSO+480 μL GVB++. All samples were analyzed in duplicate. The compound dose response curve was generated using samples prepared with 100 μL EA+50μL compound dilution+25 μL C5 dilution+25 μL sera dilution.

Test plates were prepared by adding 100 μL of EAs, 50 μL of compound dilution, 25 μL of sera dilution, and 25 μL of C5 dilution to individual wells of a 96-well tissue culture-treated clear microtitre plate (USA Scientific, Ocala, Fla.) and resuspending by pipetting up and down three times. The samples were incubated at 37° C. for one hour. At the completion of the incubation, the plates were centrifuged at 1,000× gravity for 3 minutes. 100 μl of supernatant were transferred to a new plate and the absorbance was read at 412 nm. Data was fit with a log-logit formula producing a dose-response curve and IC₅₀.

Results from RBC assay are in Table 19. The “IC₅₀” refers to the half maximal inhibitory concentration of the inhibitor needed to reduce red blood cell hemolysis by half. Compounds C5INH-0486 and C5INH-0456 were the most potent compounds tested, with IC₅₀ values of 3.0 and 7.0 nM respectively. Compounds C5INH-0476, C5INH-0488, C5INH-0395, C5INH-0315, and C5INH-0472 also exhibited IC₅₀ values below 20 nM.

TABLE 19
Red blood cell (RBC) hemolysis assay data

| | RBC Hemolysis |
|---|---|
| Compound ID | IC₅₀ (nM) |
| C5INH-0486 | 3.0 |
| C5INH-0456 | 7.0 |
| C5INH-0476 | 8.5 |
| C5INH-0488 | 12.0 |
| C5INH-0395 | 13.6 |
| C5INH-0315 | 13.7 |
| C5INH-0472 | 16.0 |
| C5INH-0287 | 20.9 |
| C5INH-0473 | 21.5 |
| C5INH-0311 | 27.0 |
| C5INH-0526 | 31.5 |
| C5INH-0525 | 34.0 |
| C5INH-0512 | 41.5 |
| C5INH-0485 | 46.9 |
| C5INH-0316 | 60.0 |
| C5INH-0321 | 61.0 |
| C5INH-0538 | 62.1 |
| C5INH-0524 | 65.0 |
| C5INH-0453 | 65.5 |
| C5INH-0371 | 67.6 |
| C5INH-0508 | 72.0 |
| C5INH-0348 | 72.5 |
| C5INH-0437 | 81.5 |
| C5INH-0491 | 83.5 |
| C5INH-0545 | 89.5 |
| C5INH-0452 | 95.5 |
| C5INH-0519 | 96.7 |
| C5INH-0356 | 111.0 |
| C5INH-0462 | 113.5 |
| C5INH-0510 | 118.5 |
| C5INH-0474 | 119.5 |
| C5INH-0516 | 126.7 |
| C5INH-0544 | 130.5 |
| C5INH-0401 | 136.5 |
| C5INH-0537 | 137.5 |
| C5INH-0490 | 144.0 |
| C5INH-0484 | 150.4 |
| C5INH-0377 | 155.7 |
| C5INH-0536 | 174.5 |
| C5INH-0489 | 176.5 |
| C5INH-0521 | 178.3 |
| C5INH-0391 | 179.5 |
| C5INH-0507 | 185.0 |
| C5INH-0310 | 203.0 |
| C5INH-0370 | 209.5 |
| C5INH-0532 | 218.5 |
| C5INH-0318 | 251.1 |
| C5INH-0298 | 269.2 |
| C5INH-0509 | 286.0 |
| C5INH-0547 | 287.0 |
| C5INH-0383 | 293.0 |
| C5INH-0450 | 298.5 |
| C5INH-0518 | 299.5 |
| C5INH-0454 | 300.5 |
| C5INH-0382 | 312.0 |
| C5INH-0504 | 318.0 |
| C5INH-0317 | 318.9 |
| C5INH-0353 | 327.9 |
| C5INH-0398 | 329.0 |
| C5INH-0294 | 343.2 |
| C5INH-0517 | 368.7 |
| C5INH-0543 | 373.0 |
| C5INH-0425 | 377.5 |
| C5INH-0496 | 400.5 |
| C5INH-0420 | 402.7 |
| C5INH-0458 | 416.0 |
| C5INH-0432 | 416.7 |
| C5INH-0357 | 417.0 |
| C5INH-0540 | 432.0 |
| C5INH-0333 | 442.0 |
| C5INH-0501 | 468.0 |
| C5INH-0336 | 473.3 |
| C5INH-0367 | 475.3 |
| C5INH-0409 | 476.5 |
| C5INH-0447 | 536.0 |
| C5INH-0324 | 550.5 |
| C5INH-0500 | 556.0 |
| C5INH-0410 | 568.5 |
| C5INH-0387 | 577.0 |
| C5INH-0369 | 591.8 |
| C5INH-0539 | 617.0 |
| C5INH-0411 | 617.6 |
| C5INH-0399 | 619.0 |
| C5INH-0350 | 653.1 |
| C5INH-0533 | 671.0 |
| C5INH-0296 | 680.4 |
| C5INH-0406 | 683.5 |
| C5INH-0390 | 697.8 |
| C5INH-0460 | 725.0 |
| C5INH-0366 | 761.5 |
| C5INH-0414 | 780.8 |
| C5INH-0326 | 784.0 |
| C5INH-0319 | 793.0 |
| C5INH-0463 | 808.0 |
| C5INH-0487 | 823.5 |
| C5INH-0339 | 878.1 |
| C5INH-0497 | 913.5 |
| C5INH-0541 | 939.5 |
| C5INH-0388 | 970.5 |
| C5INH-0340 | 991.7 |
| C5INH-0381 | 1037.5 |
| C5INH-0535 | 1044.8 |
| C5INH-0534 | 1105.5 |
| C5INH-0417 | 1125.0 |
| C5INH-0385 | 1284.0 |
| C5INH-0389 | 1311.5 |
| C5INH-0402 | 1344.5 |
| C5INH-0422 | 1372.0 |
| C5INH-0397 | 1380.5 |
| C5INH-0477 | 1455.0 |
| C5INH-0349 | 1500.0 |
| C5INH-0373 | 1524.0 |
| C5INH-0342 | 1541.5 |
| C5INH-0515 | 1600.0 |
| C5INH-0436 | 1701.5 |
| C5INH-0469 | 1796.5 |
| C5INH-0323 | 1919.5 |
| C5INH-0443 | 1989.5 |
| C5INH-0335 | 2091.5 |
| C5INH-0384 | 2180.5 |
| C5INH-0527 | 2266.5 |
| C5INH-0396 | 2327.5 |
| C5INH-0361 | 2345.0 |
| C5INH-0492 | 2650.5 |
| C5INH-0330 | 2661.5 |
| C5INH-0446 | 2712.0 |
| C5INH-0355 | 2932.5 |
| C5INH-0303 | 2942.5 |
| C5INH-0352 | 2953.5 |
| C5INH-0403 | 2987.5 |
| C5INH-0438 | 3000.5 |
| C5INH-0343 | 3038.5 |
| C5INH-0428 | 3064.0 |
| C5INH-0502 | 3093.0 |
| C5INH-0440 | 3162.0 |
| C5INH-0372 | 3267.0 |
| C5INH-0513 | 3322.0 |
| C5INH-0379 | 3631.5 |
| C5INH-0431 | 3789.0 |
| C5INH-0329 | 4012.5 |
| C5INH-0448 | 4289.0 |
| C5INH-0338 | 4372.0 |
| C5INH-0421 | 4586.0 |
| C5INH-0498 | 4635.0 |

### Example 11. Compound Analysis by Liquid Chromatography-Mass Spectrometry (LC-MS)

Inhibitor compounds were analyzed by Liquid chromatography-mass spectrometry (LC-MS) after synthesis to confirm mass-to-charge ratio (m/Z [M+H]). Results are presented in Table 20.

Analytical LCMS was performed by Waters Aquity SDS using a linear gradient of 5% to 100% B over a 5 minute gradient, and UV visualization with a diode array detector. The column used was a C18 Aquity UPLC BEH, 2.1 mm i.d. by 50 mm length, 1.7 μM with flow rate of 0.6 ml/min. Mobile phase A was water and mobile phase B was acetonitrile (0.1% TFA).

TABLE 20
LCMS assay data

| | m/z found |
|---|---|
| Compound ID | [M + H] |
| C5INH-0294 | 413.3 |
| C5INH-0296 | 417.4 |
| C5INH-0298 | 405.4 |
| C5INH-0303 | 399.4 |
| C5INH-0310 | 388.2 |
| C5INH-0311 | 389.3 |
| C5INH-0315 | 421.4 |
| C5INH-0316 | 418.4 |
| C5INH-0317 | 397.4 |
| C5INH-0318 | 444.4 |
| C5INH-0319 | 414.4 |
| C5INH-0321 | 402.4 |
| C5INH-0323 | 454.4 |
| C5INH-0324 | 469.4 |
| C5INH-0326 | 405.4 |
| C5INH-0329 | 364.3 |
| C5INH-0330 | 401.4 |
| C5INH-0333 | 373.4 |
| C5INH-0335 | 431.5 |
| C5INH-0336 | 386.5 |
| C5INH-0338 | 471.5 |
| C5INH-0339 | 401.4 |
| C5INH-0340 | 415.4 |
| C5INH-0342 | 387.4 |
| C5INH-0343 | 372.4 |
| C5INH-0348 | 417.4 |
| C5INH-0349 | 413.3 |
| C5INH-0350 | 413.4 |
| C5INH-0352 | 447.5 |
| C5INH-0353 | 426.4 |
| C5INH-0355 | 445.5 |
| C5INH-0356 | 426.4 |
| C5INH-0357 | 430.4 |
| C5INH-0361 | 411.4 |
| C5INH-0366 | 427.4 |
| C5INH-0367 | 401.3 |
| C5INH-0369 | 455.5 |
| C5INH-0370 | 510.5 |
| C5INH-0371 | 427.4 |
| C5INH-0372 | 460.4 |
| C5INH-0373 | 416.4 |
| C5INH-0377 | 494.5 |
| C5INH-0379 | 529.5 |
| C5INH-0381 | 432.2 |
| C5INH-0382 | 417.2 |
| C5INH-0383 | 445.3 |
| C5INH-0384 | 476.3 |
| C5INH-0385 | 399.3 |
| C5INH-0387 | 427.4 |
| C5INH-0388 | 427.3 |
| C5INH-0390 | 431.1 |
| C5INH-0391 | 458.5 |
| C5INH-0395 | 403.3 |
| C5INH-0396 | 440.5 |
| C5INH-0397 | 445.4 |
| C5INH-0398 | 483.4 |
| C5INH-0399 | 469.4 |
| C5INH-0401 | 388.4 |
| C5INH-0402 | 459.4 |
| C5INH-0403 | 457.4 |
| C5INH-0406 | 421.4 |
| C5INH-0409 | 518.4 |
| C5INH-0410 | 532.2 |
| C5INH-0411 | 544.4 |
| C5INH-0414 | 467.4 |
| C5INH-0417 | 388.4 |
| C5INH-0420 | 455.4 |
| C5INH-0421 | 456.4 |
| C5INH-0422 | 444.4 |
| C5INH-0425 | 441.9 |
| C5INH-0428 | 373.3 |
| C5INH-0431 | 373.5 |
| C5INH-0432 | 402.9 |
| C5INH-0436 | 439.4 |
| C5INH-0437 | 509.4 |
| C5INH-0438 | 622.5 |
| C5INH-0440 | 472.4 |
| C5INH-0443 | 386.3 |
| C5INH-0446 | 465.3 |
| C5INH-0447 | 481.4 |
| C5INH-0448 | 397.4 |
| C5INH-0450 | 408.4 |
| C5INH-0452 | 516.5 |
| C5INH-0453 | 474.3 |
| C5INH-0454 | 403.4 |
| C5INH-0456 | 403.4 |
| C5INH-0458 | 412.3 |
| C5INH-0460 | 437.4 |
| C5INH-0462 | 444.5 |
| C5INH-0463 | 440.3 |
| C5INH-0469 | 465.3 |
| C5INH-0472 | 391.4 |
| C5INH-0473 | 391.4 |
| C5INH-0474 | 493.4 |
| C5INH-0476 | 389.3 |
| C5INH-0477 | 387.3 |
| C5INH-0484 | 405.5 |
| C5INH-0485 | 403.4 |
| C5INH-0486 | 433.4 |
| C5INH-0487 | 387.3 |
| C5INH-0488 | 432.2 |
| C5INH-0489 | 474.3 |
| C5INH-0490 | 508.5 |
| C5INH-0491 | 482.5 |
| C5INH-0492 | 424.4 |
| C5INH-0496 | 414.4 |
| C5INH-0497 | 386.4 |
| C5INH-0498 | 388.4 |
| C5INH-0500 | 527.3 |
| C5INH-0501 | 447.4 |
| C5INH-0502 | 401.4 |
| C5INH-0504 | 468.4 |
| C5INH-0507 | 522.4 |
| C5INH-0508 | 524.5 |
| C5INH-0509 | 510.4 |
| C5INH-0510 | 530.4 |
| C5INH-0512 | 462.3 |
| C5INH-0513 | 382.3 |
| C5INH-0515 | 500.3 |
| C5INH-0516 | 481.3 |
| C5INH-0517 | 414.3 |
| C5INH-0518 | 487.4 |
| C5INH-05I9 | 493.4 |
| C5INH-0521 | 468.4 |
| C5INH-0524 | 482.4 |
| C5INH-0525 | 523.4 |
| C5INH-0526 | 537.5 |
| C5INH-0527 | 482.3 |
| C5INH-0532 | 488.4 |
| C5INH-0533 | 529.5 |
| C5INH-0534 | 529.5 |
| C5INH-0535 | 487.5 |
| C5INH-0536 | 468.4 |
| C5INH-0537 | 396.3 |
| C5INH-0538 | 482.5 |
| C5INH-0539 | 517.0 |
| C5INH-0540 | 488.4 |
| C5INH-0541 | 396.3 |
| C5INH-0543 | 415.4 |
| C5INH-0544 | 454.4 |
| C5INH-0545 | 494.5 |
| C5INH-0547 | 553.5 |

### Example 12. Compound Analysis by Surface Plasmon Resonance (SPR)

C5 inhibitor candidate compounds were synthesized according to standard methods known in the art [see, e.g. Morrison and Boyd in “Organic Chemistry”, 6^{th} edition, Prentice Hall (1992)] or as described in detail below, and analyzed using surface plasmon resonance (SPR) technology to generate data on the affinity, specificity, and kinetics of compound interactions with human C5 complement protein in real time without the need for labeling.

SensiQ FE SPR system (SensiQ Technologies, Oklahoma City, Okla.) was used to provide sensitive and accurate detection of binding of small molecules to the very large C5 protein (MW=195,000 Da). The chip was prepared by preconditioning the sensor according to the protocol of the SensiQ FE using 10 mM HCl, 50 mM NAOH and 0.1% SDS. The sensor chip was activated by using a mixture of fresh EDAC (1-ethyl-3-(-3-dimethylaminopropyl) carbodiimide) (Sigma Co., St. Louis, Mo.) and NHS (N-hydroxy succinimide) (Sigma Co., St. Louis, Mo.). Human C5 was surface immobilized to a Pioneer Biosensor chip via random amine coupling (>12,000 RU) which makes use of the N-terminus and ε-amino groups of lysine residues of the protein ligand. Immobilization was done by injecting 30-40 ug/ml C5 in 10 mM NaAc pH 4.5 onto designated channels at a rate of 10 μL/minute for 12 minutes, targeting RL>12000 RU for small molecules.

Compounds were diluted in DMSO in a format of 100-fold final concentration and 3-fold serial dilution (5 or 6 dilutions). The 100-fold compounds were transferred to one fold DMSO-free assay buffer in the 96-well test plate. The compound solution was injected at a rate of 60 μL/minute for 30-60 seconds, followed by 60-90 seconds dissociation time, buffer flushing and/or priming. Blank solution (1% DMSO assay buffer) was run for every 6 injections of compounds. Double reference by subtracting both blank channel and reference channel was applied for data processing. Titration of C5 binding compounds to the C5-immobilized biosensor chip surface led to interactions between C5 and potential binders, and the resulting changes of surface refractive index were sensitively measured by the system.

SPR data was analyzed with the managing software provided by SensiQ and equilibrium dissociation constant (K_{D}) values were determined for each compound at 37° C. Values obtained are presented in Table 21. Where a range of compound concentrations were analyzed, the lowest value obtained is presented. Compound CU0136 is a racemic mixture of Compound CU0186 and CU0187. Numbers in parenthesis following compound numbers indicate alternate enantiomers (as distinguished by retention time during chromatographical separation).

TABLE 21
Surface plasmon resonance (SPR) data

| | K_{D} |
|---|---|
| Compound Number | (nM) |
| CU0104 | 0.6 |
| CU0231 | 0.9 |
| CU0109 | 1.1 |
| CU0108 | 1.2 |
| CU0232 | 1.3 |
| CU0021 | 1.5 |
| CU0228 | 1.6 |
| CU0030 | 1.8 |
| CU0105 | 1.9 |
| CU0107 | 2.0 |
| CU0106 | 2.0 |
| CU0110 | 2.0 |
| CU0100 | 2.1 |
| CU0029 | 2.1 |
| CU0101 | 2.4 |
| CU0005 | 2.5 |
| CU0112 | 2.6 |
| CU0022 | 2.9 |
| CU0020 | 3.0 |
| CU0111 | 3.4 |
| CU0120 | 3.6 |
| CU0235 | 3.8 |
| CU0123 | 4.2 |
| CU0024 | 5.0 |
| CU0025 | 5.0 |
| CU0028 | 5.0 |
| CU0031 | 6.0 |
| CU0124 | 6.4 |
| CU0008 | 6.5 |
| CU0039(2) | 6.5 |
| CU0115 | 7.8 |
| CU0051 | 8.0 |
| CU0117 | 8.3 |
| CU0050 | 9.0 |
| CU0127 | 9.5 |
| CU0122 | 9.6 |
| CU0116 | 10.3 |
| CU0004 | 10.5 |
| CU0053 | 10.5 |
| CU0055 | 10.5 |
| CU0063 | 10.5 |
| CU0118 | 12.8 |
| CU0061 | 13.0 |
| CU0059 | 13.5 |
| CU0003 | 14.0 |
| CU0027 | 14.0 |
| CU0060 | 14.0 |
| CU0019 | 14.5 |
| CU0037 | 15.5 |
| CU0046 | 15.5 |
| CU0043 | 16.5 |
| CU0041 | 17.0 |
| CU0062 | 17.5 |
| CU0057 | 18.0 |
| CU0048 | 18.5 |
| CU0049 | 21.0 |
| CU0056 | 23.0 |
| CU0129 | 23.8 |
| CU0036 | 25.0 |
| CU0045 | 26.0 |
| CU0018 | 27.5 |
| CU0121 | 27.5 |
| CU0039(1) | 28.0 |
| CU0001 | 29.5 |
| CU0035 | 33.5 |
| CU0044 | 35.5 |
| CU0032 | 47.5 |
| CU0033 | 62.0 |
| CU0010 | 74.5 |
| CU0002 | 83.5 |
| CU0042 | 106.0 |
| CU0014 | 174.0 |
| CU0017 | 188.0 |
| CU0012 | 326.0 |
| CU0065 | 370.0 |
| CU0064 | 394.0 |
| CU0013 | 454.0 |
| CU0011 | 816.0 |

### Example 13. Compound Analysis by Red Blood Cell (RBC) Hemolysis Assay

Sheep red blood cells coated with rabbit anti-sheep erythrocyte antiserum (EA cells: Complement Technology, Tyler, Tex.) were used to assay compound inhibitory activity of the classical complement activation pathway. Briefly, the EA cells were washed once and resuspended in the same volume of GVB++ buffer (Complement Technology, Tyler, Tex.). 25 μL of EA cells were then distributed into each well of 384-well tissue culture plates using Apricot iPipette Pro (Apricot Designs; Covina, Calif.). Compounds were tested in 10 points of final concentrations ranging from 16.67 μM to 1.65 μM in a 6-fold titration series. Compounds were dispensed into 384-well plates from 6.7 mM and 3.35 μM DMSO working stocks using an HP Digital Dispenser (HP; Corvallis, Oreg.). The reactions also contained 1.5% (v/v) C5-depleted human serum (Complement Technology). Hemolysis was induced by addition of human C5 (Complement Technology) at a concentration of 0.5 nM and plates were incubated for 1 hour at 37° C. in a cell culture incubator. The extent of hemolysis was measured by ability of released hemoglobin to catalyze luminol in the presence of hydrogen peroxide. Luminescence was then measured using a plate reader.

Luminescence measurements were used to prepare a dose-response curve. From the curve, the half maximal inhibitory concentration (IC₅₀) for each compound was determined, where the IC₅₀ represents the concentration of each compound needed to reduce red blood cell hemolysis by half. Results are presented in Table 22. Compound CU0136 is a racemic mixture of Compound CU0186 and CU0187. Numbers in parenthesis following compound numbers indicate alternate enantiomers (as distinguished by retention time during chromatographical separation).

TABLE 22
Red blood cell (RBC) hemolysis assay data

| | IC₅₀ |
|---|---|
| Compound Number | (nM) |
| CU0205 | 5.1 |
| CU0243 | 5.5 |
| CU0244 | 6.7 |
| CU0201 | 7.4 |
| CU0258 | 7.5 |
| CU0242 | 8.2 |
| CU0028 | 8.9 |
| CU0200 | 8.9 |
| CU0247 | 9.8 |
| CU0137 | 10.0 |
| CU0246 | 10.6 |
| CU0241 | 10.7 |
| CU0202 | 10.8 |
| CU0261 | 11.0 |
| CU0239 | 11.2 |
| CU0173 | 11.4 |
| CU0120 | 11.8 |
| CU0206 | 11.9 |
| CU0004 | 12.1 |
| CU0245 | 12.2 |
| CU0136 | 12.5 |
| CU0146 | 12.5 |
| CU0024 | 12.9 |
| CU0141 | 13.1 |
| CU0207 | 13.1 |
| CU0007 | 13.3 |
| CU0025 | 13.6 |
| CU0135 | 14.5 |
| CU0021 | 14.5 |
| CU0187 | 14.8 |
| CU0029 | 15.0 |
| CU0145 | 15.0 |
| CU0224 | 15.4 |
| CU0211 | 15.6 |
| CU0031 | 15.7 |
| CU0226 | 15.7 |
| CU0140 | 15.8 |
| CU0257 | 16.0 |
| CU0134 | 16.1 |
| CU0110 | 16.4 |
| CU0005 | 17.0 |
| CU0022 | 17.1 |
| CU0122 | 17.1 |
| CU0123 | 17.1 |
| CU0020 | 17.2 |
| CU0050 | 18.0 |
| CU0142 | 18.3 |
| CU0217 | 18.3 |
| CU0109 | 18.6 |
| CU0254 | 18.7 |
| CU0186 | 18.8 |
| CU0192 | 19.1 |
| CU0255 | 19.4 |
| CU0006 | 19.5 |
| CU0108 | 19.5 |
| CU0188 | 19.9 |
| CU0170 | 20.1 |
| CU0231 | 20.3 |
| CU0213 | 20.4 |
| CU0023 | 20.7 |
| CU0100 | 21.0 |
| CU0177 | 21.1 |
| CU0196 | 21.1 |
| CU0214 | 21.1 |
| CU0204 | 21.6 |
| CU0232 | 21.6 |
| CU0228 | 21.8 |
| CU0057 | 22.4 |
| CU0027 | 22.5 |
| CU0019 | 22.6 |
| CU0154 | 22.6 |
| CU0161 | 22.9 |
| CU0116 | 23.2 |
| CU0053 | 23.3 |
| CU0143 | 23.3 |
| CU0160 | 23.5 |
| CU0212 | 23.6 |
| CU0234 | 23.7 |
| CU0104 | 23.8 |
| CU0149 | 24.1 |
| CU0260 | 25.0 |
| CU0111 | 25.2 |
| CU0112 | 25.9 |
| CU0124 | 26.0 |
| CU0101 | 26.3 |
| CU0178 | 26.7 |
| CU0030 | 26.9 |
| CU0216 | 27.0 |
| CU0158 | 27.8 |
| CU0115 | 27.9 |
| CU0175 | 28.1 |
| CU0063 | 28.3 |
| CU0003 | 28.4 |
| CU0218 | 28.9 |
| CU0225 | 29.1 |
| CU0235 | 29.4 |
| CU0043 | 29.5 |
| CU0039(1) | 30.0 |
| CU0203 | 30.0 |
| CU0039(2) | 30.1 |
| CU0127 | 30.2 |
| CU0035 | 30.8 |
| CU0055 | 31.2 |
| CU0197 | 31.6 |
| CU0184 | 32.4 |
| CU0117 | 32.5 |
| CU0045 | 32.8 |
| CU0059 | 32.9 |
| CU0210 | 32.9 |
| CU0062 | 33.4 |
| CU0251 | 34.0 |
| CU0215 | 34.1 |
| CU0262 | 34.6 |
| CU0010 | 34.9 |
| CU0118 | 35.3 |
| CU0001 | 35.6 |
| CU0165 | 36.0 |
| CU0148 | 36.0 |
| CU0174 | 36.7 |
| CU0172 | 37.3 |
| CU0107 | 38.0 |
| CU0121 | 38.9 |
| CU0046 | 38.9 |
| CU0167 | 39.9 |
| CU0060 | 40.3 |
| CU0018 | 40.6 |
| CU0190 | 41.4 |
| CU0253 | 41.9 |
| CU0002 | 42.0 |
| CU0240 | 42.2 |
| CU0155 | 43.7 |
| CU0106 | 43.9 |
| CU0237 | 43.9 |
| CU0049 | 44.7 |
| CU0193 | 45.0 |
| CU0171 | 45.1 |
| CU0051 | 45.9 |
| CU0147 | 46.4 |
| CU0037 | 47.1 |
| CU0056 | 48.8 |
| CU0195 | 49.0 |
| CU0032 | 51.8 |
| CU0119 | 52.0 |
| CU0061 | 52.3 |
| CU0102 | 53.7 |
| CU0113 | 54.8 |
| CU0041 | 55.2 |
| CU0209 | 55.2 |
| CU0138 | 55.4 |
| CU0048 | 57.3 |
| CU0208 | 59.1 |
| CU0230 | 60.5 |
| CU0128 | 60.6 |
| CU0238 | 61.0 |
| CU0052 | 61.4 |
| CU0129 | 61.5 |
| CU0026 | 62.5 |
| CU0256 | 62.7 |
| CU0008 | 62.9 |
| CU0042 | 63.1 |
| CU0040(1) | 66.5 |
| CU0222 | 69.9 |
| CU0198 | 70.1 |
| CU0101 | 73.5 |
| CU0125 | 73.6 |
| CU0259 | 75.0 |
| CU0040(2) | 76.3 |
| CU0014 | 77.6 |
| CU0133 | 82.2 |
| CU0169 | 82.4 |
| CU0150 | 87.6 |
| CU0044 | 87.7 |
| CU0191 | 87.9 |
| CU0229 | 88.9 |
| CU0139 | 91.7 |
| CU0166 | 92.1 |
| CU0194 | 94.6 |
| CU0159 | 98.8 |
| CU0114 | 99.1 |
| CU0248 | 101.0 |
| CU0017 | 106.3 |
| CU0144 | 109.1 |
| CU0176 | 111.3 |
| CU0054 | 117.5 |
| CU0181 | 118.9 |
| CU0036 | 120.7 |
| CU0065 | 123.1 |
| CU0012 | 128.0 |
| CU0105 | 130.4 |
| CU0033 | 135.6 |
| CU0152 | 145.9 |
| CU0157 | 146.4 |
| CU0130 | 146.8 |
| CU0131 | 151.2 |
| CU0126 | 152.8 |
| CU0179 | 155.7 |
| CU0185 | 183.4 |
| CU0009 | 202.8 |
| CU0236 | 203.3 |
| CU0250 | 216.8 |
| CU0219 | 221.3 |
| CU0168 | 232.5 |
| CU0153 | 233.0 |
| CU0183 | 237.5 |
| CU0156 | 241.5 |
| CU0066 | 243.4 |
| CU0220 | 253.5 |
| CU0038 | 266.6 |
| CU0132 | 301.5 |
| CU0227 | 362.1 |
| CU0199 | 370.4 |
| CU0249 | 385.9 |
| CU0223 | 399.5 |
| CU0180 | 411.2 |
| CU0189 | 412.6 |
| CU0015 | 519.1 |
| CU0164 | 592.0 |
| CU0013 | 601.8 |
| CU0151 | 706.7 |
| CU0221 | 741.3 |
| CU0064 | 799.0 |
| CU0252 | 825.4 |
| CU0011 | 886.4 |
| CU0182 | 935.6 |
| CU0067 | 945.6 |
| CU0163 | 1000.0 |
| CU0034 | 1231.9 |
| CU0058 | 2486.5 |
| CU0016 | 2623.5 |
| CU0162 | 2865.9 |
| CU0047 | 3137.8 |
| CU0233 | 4255.4 |

### Example 14. Compound Analysis by Liquid Chromatography-Mass Spectrometry (LC-MS)

Compounds were analyzed by Liquid chromatography-mass spectrometry (LC-MS) after synthesis to confirm mass-to-charge ratio (m/Z [M+H]). Analytical LCMS was performed by Waters Aquity SDS using a linear gradient of 5% to 100% B over a 5 minute gradient, and UV visualization with a diode array detector. The column used was a C18 Aquity UPLC BEH, 2.1 mm i.d. by 50 mm length, 1.7 μM with flow rate of 0.6 ml/min. Mobile phase A was water and mobile phase B was acetonitrile (0.1% TFA). Results are shown in Table 23. Compound CU0136 is a racemic mixture of Compound CU0186 and CU0187. Numbers in parenthesis following compound numbers indicate alternate enantiomers (as distinguished by retention time during chromatographical separation).

TABLE 23
LCMS assay data

| | m/z found |
|---|---|
| Compound Number | [M + H] |
| CU0001 | 399.5 |
| CU0002 | 418.3 |
| CU0003 | 431.3 |
| CU0004 | 457.2 |
| CU0005 | 498.4 |
| CU0006 | 523.3 |
| CU0007 | 504.5 |
| CU0008 | 540.5 |
| CU0009 | 403.1 |
| CU0010 | 417.1 |
| CU0011 | 423.3 |
| CU0012 | 435.3 |
| CU0013 | 421.3 |
| CU0014 | 435.4 |
| CU0015 | 423.3 |
| CU0016 | 399.1 |
| CU0017 | 401.1 |
| CU0018 | 414.3 |
| CU0019 | 423.4 |
| CU0020 | 519.2 |
| CU0021 | 535.8 |
| CU0022 | 549.4 |
| CU0023 | 513.4 |
| CU0024 | 517.5 |
| CU0025 | 507.4 |
| CU0026 | 517.5 |
| CU0027 | 543.3 |
| CU0028 | 562.4 |
| CU0029 | 549.5 |
| CU0030 | 577.6 |
| CU0031 | 593.5 |
| CU0032 | 423.3 |
| CU0033 | 494.2 |
| CU0034 | 504.2 |
| CU0035 | 508.4 |
| CU0036 | 518.2 |
| CU0037 | 522.4 |
| CU0038 | 536.5 |
| CU0039(1) | 520.4 |
| CU0039(2) | 520.9 |
| CU0040(1) | 534.5 |
| CU0040(2) | 534.5 |
| CU0041 | 564.6 |
| CU0042 | 536.5 |
| CU0043 | 550.2 |
| CU0044 | 560.2 |
| CU0045 | 564.5 |
| CU0046 | 594.6 |
| CU0047 | 580.5 |
| CU0048 | 594.2 |
| CU0049 | 608.7 |
| CU0050 | 607.6 |
| CU0051 | 633.6 |
| CU0052 | 550.2 |
| CU0053 | 564.2 |
| CU0054 | 574.2 |
| CU0055 | 578.2 |
| CU0056 | 589.6 |
| CU0057 | 607.6 |
| CU0058 | 549.5 |
| CU0059 | 563.2 |
| CU0060 | 577.3 |
| CU0061 | 587.2 |
| CU0062 | 591.6 |
| CU0063 | 591.2 |
| CU0064 | 413.4 |
| CU0065 | 427.8 |
| CU0066 | 438.1 |
| CU0067 | 452.3 |
| CU0100 | 554.9 |
| CU0101 | 553.5 |
| CU0102 | 471.5 |
| CU0103 | 514.1 |
| CU0104 | 567.6 |
| CU0105 | 584.6 |
| CU0106 | 571.6 |
| CU0107 | 581.6 |
| CU0108 | 557.5 |
| CU0109 | 595.6 |
| CU0110 | 581.6 |
| CU0111 | 534.5 |
| CU0112 | 590.5 |
| CU0113 | 508.5 |
| CU0114 | 564.5 |
| CU0115 | 502.5 |
| CU0116 | 544.5 |
| CU0117 | 558.5 |
| CU0118 | 528.5 |
| CU0119 | 523.5 |
| CU0120 | 495.5 |
| CU0121 | 498.6 |
| CU0122 | 512.9 |
| CU0123 | 510.5 |
| CU0124 | 526.5 |
| CU0125 | 539.6 |
| CU0126 | 523.6 |
| CU0127 | 564.7 |
| CU0128 | 482.5 |
| CU0129 | 535.6 |
| CU0130 | 541.7 |
| CU0131 | 528.6 |
| CU0132 | 486.5 |
| CU0133 | 499.6 |
| CU0134 | 585.6 |
| CU0135 | 599.6 |
| CU0136 | 597.7 |
| CU0137 | 499.7 |
| CU0138 | 553.7 |
| CU0139 | 506.7 |
| CU0140 | 534.5 |
| CU0141 | 631.7 |
| CU0142 | 617.6 |
| CU0143 | 449.5 |
| CU0144 | 483.0 |
| CU0145 | 576.6 |
| CU0146 | 590.6 |
| CU0147 | 508.8 |
| CU0148 | 509.2 |
| CU0149 | 514.4 |
| CU0150 | 564.6 |
| CU0151 | 494.6 |
| CU0152 | 576.7 |
| CU0153 | 491.3 |
| CU0154 | 443.3 |
| CU0155 | 494.6 |
| CU0156 | 495.1 |
| CU0157 | 577.2 |
| CU0158 | 576.7 |
| CU0159 | 456.7 |
| CU0160 | 484.6 |
| CU0161 | 535.9 |
| CU0162 | 467.2 |
| CU0163 | 451.5 |
| CU0164 | 451.5 |
| CU0165 | 431.3 |
| CU0166 | 447.3 |
| CU0167 | 429.4 |
| CU0168 | 443.4 |
| CU0169 | 427.3 |
| CU0170 | 633.6 |
| CU0171 | 534.6 |
| CU0172 | 511.6 |
| CU0173 | 580.7 |
| CU0174 | 590.6 |
| CU0175 | 633.7 |
| CU0176 | 449.5 |
| CU0177 | 581.1 |
| CU0178 | 539.7 |
| CU0179 | 427.4 |
| CU0180 | 465.4 |
| CU0181 | 465.4 |
| CU0182 | 465.4 |
| CU0183 | 479.2 |
| CU0184 | 483.3 |
| CU0185 | 457.3 |
| CU0186 | 597.6 |
| CU0187 | 597.6 |
| CU0188 | 470.2 |
| CU0189 | 554.3 |
| CU0190 | 538.2 |
| CU0191 | 539.3 |
| CU0192 | 514.1 |
| CU0193 | 465.3 |
| CU0194 | 429.3 |
| CU0195 | 521.3 |
| CU0196 | 584.7 |
| CU0197 | 620.6 |
| CU0198 | 469.4 |
| CU0199 | 518.4 |
| CU0200 | 523.0 |
| CU0201 | 442.1 |
| CU0202 | 526.3 |
| CU0203 | 499.4 |
| CU0204 | 528.2 |
| CU0205 | 457.2 |
| CU0206 | 457.2 |
| CU0207 | 631.7 |
| CU0208 | 494.3 |
| CU0209 | 495.3 |
| CU0210 | 452.2 |
| CU0211 | 509.5 |
| CU0212 | 509.4 |
| CU0213 | 550.4 |
| CU0214 | 588.4 |
| CU0215 | 479.3 |
| CU0216 | 471.3 |
| CU0217 | 445.3 |
| CU0218 | 445.3 |
| CU0219 | 479.3 |
| CU0220 | 533.3 |
| CU0221 | 535.3 |
| CU0222 | 485.3 |
| CU0223 | 519.3 |
| CU0224 | 584.9 |
| CU0225 | 443.3 |
| CU0226 | 524.4 |
| CU0227 | 481.1 |
| CU0228 | 563.6 |
| CU0229 | 521.6 |
| CU0230 | 525.5 |
| CU0231 | 521.5 |
| CU0232 | 525.5 |
| CU0233 | 521.7 |
| CU0234 | 504.6 |
| CU0235 | 533.6 |
| CU0236 | 521.5 |
| CU0237 | 525.5 |
| CU0238 | 521.7 |
| CU0239 | 589.6 |
| CU0240 | 458.5 |
| CU0241 | 545.6 |
| CU0242 | 590.7 |
| CU0243 | 562.6 |
| CU0244 | 576.6 |
| CU0245 | 549.6 |
| CU0246 | 576.6 |
| CU0247 | 606.7 |
| CU0248 | 507.4 |
| CU0249 | 535.6 |
| CU0250 | 536.6 |
| CU0251 | 519.6 |
| CU0252 | 528.4 |
| CU0253 | 560.4 |
| CU0254 | 520.6 |
| CU0255 | 605.7 |
| CU0256 | 598.5 |
| CU0257 | 591.7 |
| CU0258 | 522.6 |
| CU0259 | 534.4 |
| CU0260 | 594.6 |
| CU0261 | 543.4 |
| CU0262 | 544.6 |

### Example 15. Drug-Metabolism-and-Pharmacokinetics (DMPK)

Single intravenous (IV) and oral dose (PO, per oral) administration of C5 inhibitor compounds is carried out in rats. Rats are then analyzed for Drug-Metabolism-and-Pharmacokinetic (DMPK) properties, used to determine compound pharmacokinetics and oral bioavailability.

1 mg/mL compound solutions are prepared in 5% DMSO: 20% HP-Beta-CD. Fasted male Sprague Dawley rats are dosed with solutions at 1 mg/kg by IV and 10 mg/kg PO. Analysis of compound DMPK properties is used to determine bioavailability.

### Example 16. Hemolysis Inhibition with Paroxysmal Nocturnal Hemoglobinuria Patient Cells

Flow cytometry studies are carried out to assess compound hemolysis inhibition with CD59-deficient RBCs from patients with paroxysmal nocturnal hemoglobinuria (PNH). RBCs collected from PNH patients are washed three times with Alsever's solution, followed by pelleting and re-suspending in GVB++ buffer (Complement Technology, Tyler, Tex.) in a ratio of 1:2. To induce hemolysis, donor-matched serum is acidified to pH 6.4 with HCl. Compounds, serum, and RBCs, 2.5% volume per volume (v/v), are incubated for 18 hours at 37° C. After incubation, cells are washed and re-suspended in 1 ml fluorescence-associated cell sorting (FACS) buffer (0.1% BSA IgG-free in PBS, 0.1% Sodium Azide). Then, anti-CD59 antibody conjugated with phycoerythrin is added at a final concentration of 0.25 μg/ml to 100 μl of cell suspension and incubated at 4° C. for 30 minutes. Cells are then washed twice with cold FACS buffer, re-suspended in FACS buffer and analyzed with a BD Accuri C6 Flow Cytometer (BD Biosciences, San Jose, Calif.) for CD59 levels. The level of CD59-positive cells is monitored as a measure of complement-mediated hemolysis of PNH type III cells. A negative control using non-acidified serum is used to establish a baseline of CD59 expression under non-hemolytic conditions. When acidified serum is introduced, the level of CD59 expression decreases, consistent with RBC hemolysis. Hemolysis is blocked in the presence of eculizumab, which is a known antibody-based C5 inhibitor.

Similar experiments are conducted using increasing concentrations of C5 inhibitor compounds to assess inhibition in a dose-dependent manner.

### Example 17. Synthesis of Cyclic Urea Compounds and Intermediates

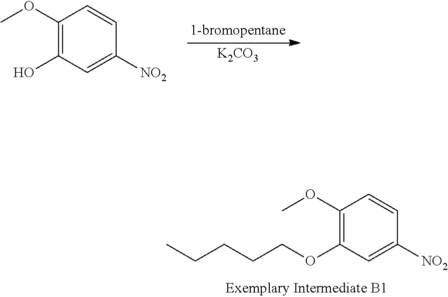

A solution which included a phenol reactant (2-methoxy-5-nitrophenol, 100.0 g, 0.59 mol) and a bromide reactant (1-bromopentane, 117.2 g, 0.76 mol, 1.3 eq) in a reaction solvent (N,N-dimethylformamide, 1.0 L) is provided. Potassium carbonate (122.5 g, 0.89 mol, 1.5 eq) is added at room temperature. The mixture is heated to 80° C. and stirred overnight. The reaction mixture is cooled to room temperature, diluted with water (3.0 L), then extracted with ethyl acetate (3×2.0 L). The combined organic phases are washed with brine (3×3.0 L), dried over anhydrous sodium sulfate, then filtered and concentrated in vacuo to a volume of about 300 mL. The residue is diluted with hexane (1.0 L) and stirred for 10 minutes to precipitate a white solid. The solids are collected by filtration and dried under vacuum to afford a compound, Exemplary Intermediate B1 (1-methoxy-4-nitro-2-(pentyloxy)benzene, 119.7 g, 85% yield).

The following compounds are prepared in a similar manner as Exemplary Intermediate B1, as described above.

| Phenol | Bromide | Compound |
|---|---|---|
| 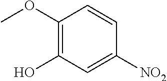 | 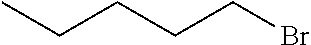 | 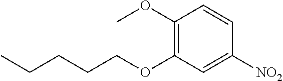 [SMILES: CCCCCOc1cc([N+](=O)[O-])ccc1OC] |
| | | Exemplary Intermediate B1 |
| 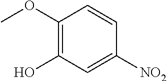 | 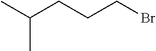 | 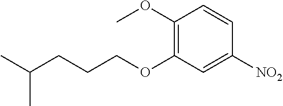 [SMILES: COc1ccc([N+](=O)[O-])cc1OCCCC(C)C] |
| 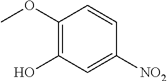 | 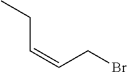 | 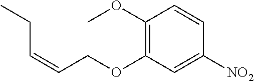 [SMILES: CC/C=C\COc1cc([N+](=O)[O-])ccc1OC] |
| 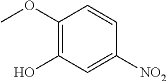 | 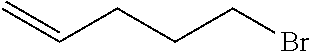 | 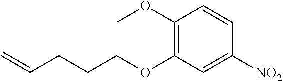 [SMILES: C=CCCCOc1cc([N+](=O)[O-])ccc1OC] |
| 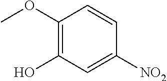 | 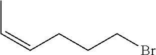 | 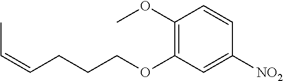 [SMILES: C/C=C\CCCOc1cc([N+](=O)[O-])ccc1OC] |
| 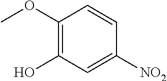 | 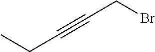 | 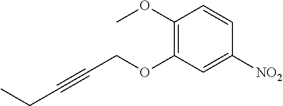 [SMILES: CCC#CCOc1cc([N+](=O)[O-])ccc1OC] |
| 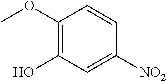 | 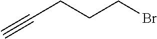 | 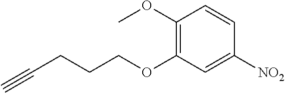 [SMILES: C#CCCCOc1cc([N+](=O)[O-])ccc1OC] |
| 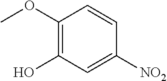 | 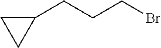 | 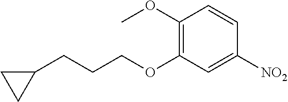 [SMILES: COc1ccc([N+](=O)[O-])cc1OCCCC1CC1] |
| 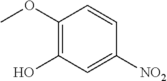 | 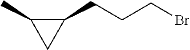 | 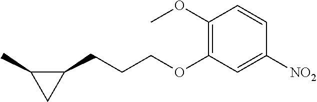 [SMILES: COc1ccc([N+](=O)[O-])cc1OCCC[C@H]1C[C@H]1C] |
| | | Racemic |
| 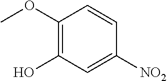 | 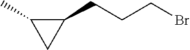 | 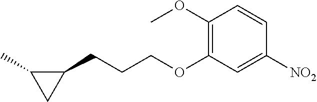 [SMILES: COc1ccc([N+](=O)[O-])cc1OCCC[C@H]1C[C@@H]1C] |
| | | Racemic |
| 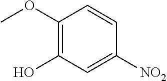 | 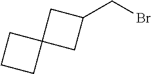 | 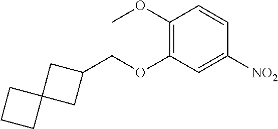 [SMILES: COc1ccc([N+](=O)[O-])cc1OCC1CC2(CCC2)C1] |
| 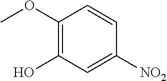 | 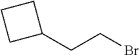 | 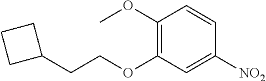 [SMILES: COc1ccc([N+](=O)[O-])cc1OCCC1CCC1] |
| 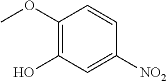 | 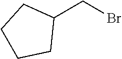 | 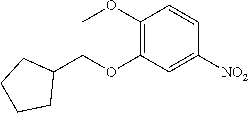 [SMILES: COc1ccc([N+](=O)[O-])cc1OCC1CCCC1] |
| 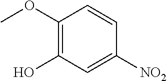 | 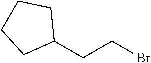 | 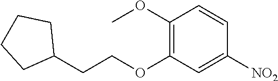 [SMILES: COc1ccc([N+](=O)[O-])cc1OCCC1CCCC1] |
| 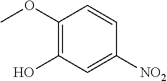 | 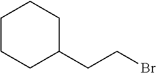 | 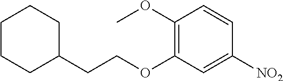 [SMILES: COc1ccc([N+](=O)[O-])cc1OCCC1CCCCC1] |
| 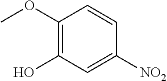 | 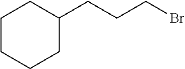 | 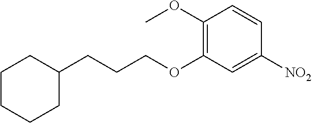 [SMILES: COc1ccc([N+](=O)[O-])cc1OCCCC1CCCCC1] |
| 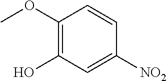 | 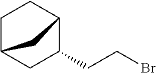 | 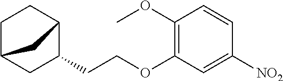 [SMILES: COc1ccc([N+](=O)[O-])cc1OCC[C@@H]1C[C@@H]2CC[C@H]1C2] |
| 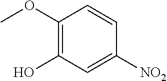 | 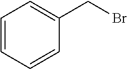 | 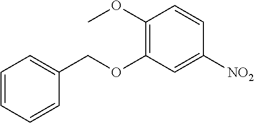 [SMILES: COc1ccc([N+](=O)[O-])cc1OCc1ccccc1] |
| 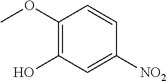 | 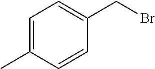 | 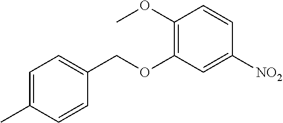 [SMILES: COc1ccc([N+](=O)[O-])cc1OCc1ccc(C)cc1] |
| 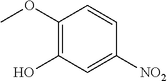 | 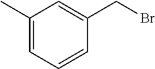 | 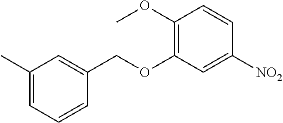 [SMILES: COc1ccc([N+](=O)[O-])cc1OCc1cccc(C)c1] |
| 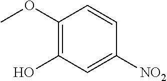 | 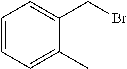 | 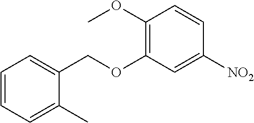 [SMILES: COc1ccc([N+](=O)[O-])cc1OCc1ccccc1C] |
| 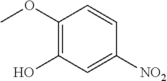 | 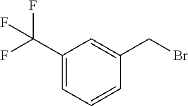 [SMILES: FC(F)(F)c1cccc(CBr)c1] | 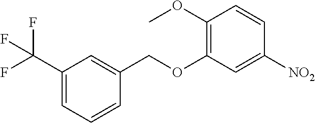 [SMILES: COc1ccc([N+](=O)[O-])cc1OCc1cccc(C(F)(F)F)c1] |
| 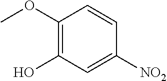 | 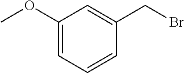 | 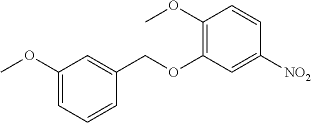 [SMILES: COc1cccc(COc2cc([N+](=O)[O-])ccc2OC)c1] |
| 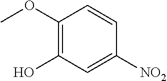 | 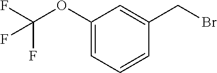 [SMILES: FC(F)(F)Oc1cccc(CBr)c1] | 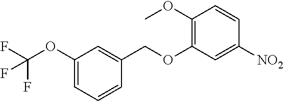 [SMILES: COc1ccc([N+](=O)[O-])cc1OCc1cccc(OC(F)(F)F)c1] |
| 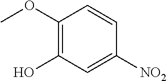 | 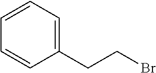 | 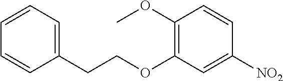 [SMILES: COc1ccc([N+](=O)[O-])cc1OCCc1ccccc1] |
| 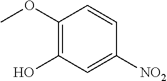 | 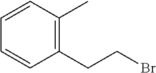 | 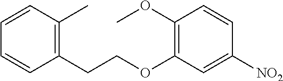 [SMILES: COc1ccc([N+](=O)[O-])cc1OCCc1ccccc1C] |
| 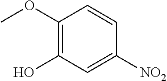 | 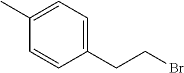 | 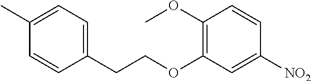 [SMILES: COc1ccc([N+](=O)[O-])cc1OCCc1ccc(C)cc1] |
| 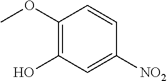 | 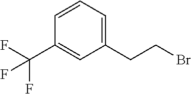 [SMILES: FC(F)(F)c1cccc(CCBr)c1] | 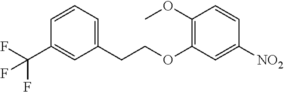 [SMILES: COc1ccc([N+](=O)[O-])cc1OCCc1cccc(C(F)(F)F)c1] |
| 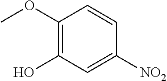 | 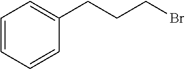 | 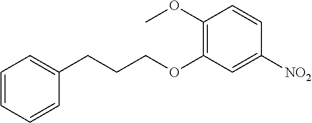 [SMILES: COc1ccc([N+](=O)[O-])cc1OCCCc1ccccc1] |
| 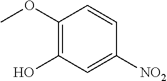 | 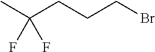 | 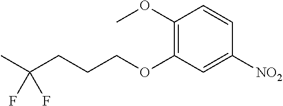 [SMILES: COc1ccc([N+](=O)[O-])cc1OCCCC(C)(F)F] |
| 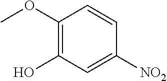 | 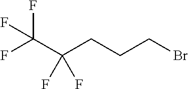 [SMILES: FC(F)(F)C(F)(F)CCCBr] | 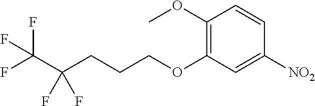 [SMILES: COc1ccc([N+](=O)[O-])cc1OCCCC(F)(F)C(F)(F)F] |
| 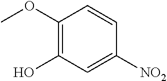 | 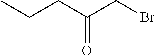 | 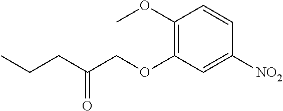 [SMILES: CCCC(=O)COc1cc([N+](=O)[O-])ccc1OC] |
| | | Exemplary Intermediate B6 |
| 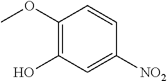 | 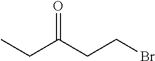 | 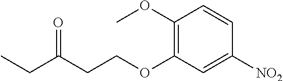 [SMILES: CCC(=O)CCOc1cc([N+](=O)[O-])ccc1OC] |
| 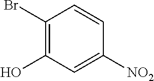 [SMILES: O=[N+]([O-])c1ccc(Br)c(O)c1] | 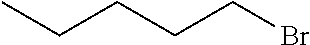 | 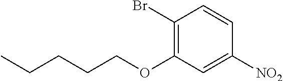 [SMILES: CCCCCOc1cc([N+](=O)[O-])ccc1Br] |
| | | Exemplary Intermediate B2 |
| 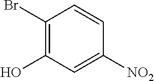 [SMILES: O=[N+]([O-])c1ccc(Br)c(O)c1] | 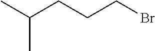 | 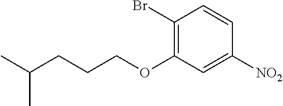 [SMILES: CC(C)CCCOc1cc([N+](=O)[O-])ccc1Br] |
| 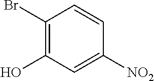 [SMILES: O=[N+]([O-])c1ccc(Br)c(O)c1] | 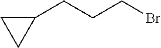 | 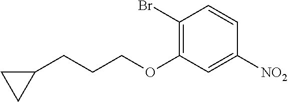 [SMILES: O=[N+]([O-])c1ccc(Br)c(OCCCC2CC2)c1] |
| 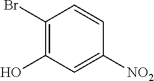 [SMILES: O=[N+]([O-])c1ccc(Br)c(O)c1] | 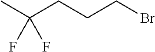 | 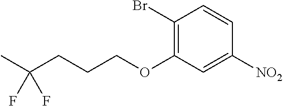 [SMILES: CC(F)(F)CCCOc1cc([N+](=O)[O-])ccc1Br] |
| 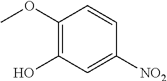 | 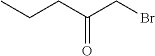 | 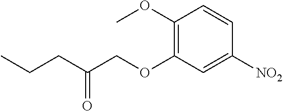 [SMILES: CCCC(=O)COc1cc([N+](=O)[O-])ccc1OC] |
| | | Exemplary Intermediate B6 |

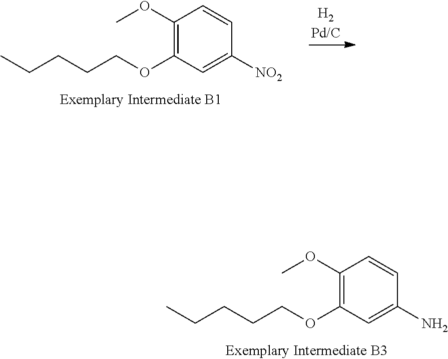 [SMILES: CCCCCOc1cc([N+](=O)[O-])ccc1OC ; CCCCCOc1cc(N)ccc1OC]

Exemplary Intermediate B1 (1-methoxy-4-nitro-2-(pentyloxy)benzene, 119.5 g, 0.50 mol) is dissolved in methanol (1.5 L), and 10 wt % palladium on carbon (10 g) is added. The mixture is stirred overnight under an atmosphere of hydrogen. The reaction mixture is filtered through Celite, and the filter bed is washed with methanol (500 mL). The filtered solution is concentrated to dryness to afford Exemplary Intermediate B3 (4-methoxy-3-(pentyloxy)aniline, 95.0 g, 91% yield).

The following compounds are prepared in a similar manner as Exemplary Intermediate B3, as described above.

| Nitro | Compound |
|---|---|
| 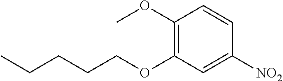 [SMILES: CCCCCOc1cc([N+](=O)[O-])ccc1OC] | 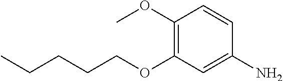 |
| Exemplary Intermediate B1 | Exemplary Intermediate B3 |
| 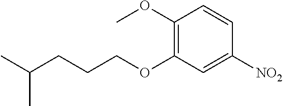 [SMILES: COc1ccc([N+](=O)[O-])cc1OCCCC(C)C] | 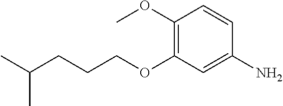 [SMILES: COc1ccc(N)cc1OCCCC(C)C] |
| 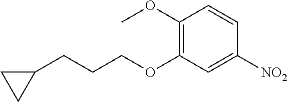 [SMILES: COc1ccc([N+](=O)[O-])cc1OCCCC1CC1] | 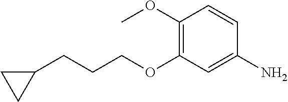 [SMILES: COc1ccc(N)cc1OCCCC1CC1] |
| 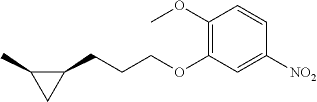 [SMILES: COc1ccc([N+](=O)[O-])cc1OCCC[C@H]1C[C@H]1C] | 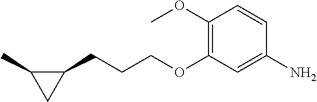 [SMILES: COc1ccc(N)cc1OCCC[C@H]1C[C@H]1C] |
| | Racemic |
| 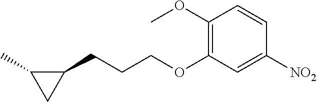 [SMILES: COc1ccc([N+](=O)[O-])cc1OCCC[C@H]1C[C@@H]1C] | 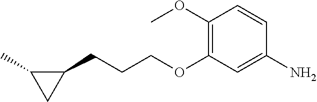 [SMILES: COc1ccc(N)cc1OCCC[C@H]1C[C@@H]1C] |
| | Racemic |
| 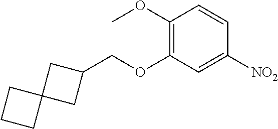 [SMILES: COc1ccc([N+](=O)[O-])cc1OCC1CC2(CCC2)C1] | 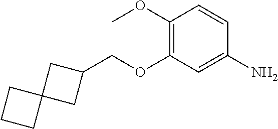 [SMILES: COc1ccc(N)cc1OCC1CC2(CCC2)C1] |
| 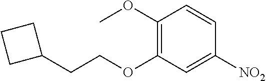 [SMILES: COc1ccc([N+](=O)[O-])cc1OCCC1CCC1] | 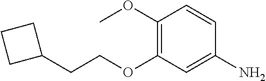 [SMILES: COc1ccc(N)cc1OCCC1CCC1] |
| 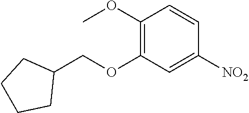 [SMILES: COc1ccc([N+](=O)[O-])cc1OCC1CCCC1] | 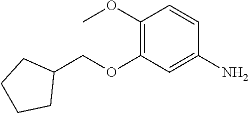 [SMILES: COc1ccc(N)cc1OCC1CCCC1] |
| 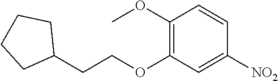 [SMILES: COc1ccc([N+](=O)[O-])cc1OCCC1CCCC1] | 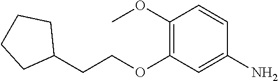 [SMILES: COc1ccc(N)cc1OCCC1CCCC1] |
| 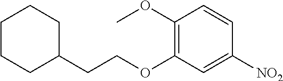 [SMILES: COc1ccc([N+](=O)[O-])cc1OCCC1CCCCC1] | 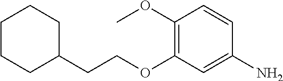 [SMILES: COc1ccc(N)cc1OCCC1CCCCC1] |
| 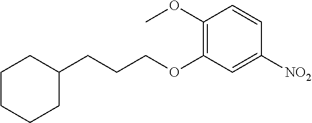 [SMILES: COc1ccc([N+](=O)[O-])cc1OCCCC1CCCCC1] | 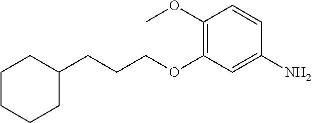 [SMILES: COc1ccc(N)cc1OCCCC1CCCCC1] |
| 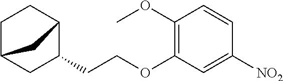 [SMILES: COc1ccc([N+](=O)[O-])cc1OCC[C@@H]1C[C@@H]2CC[C@H]1C2] | 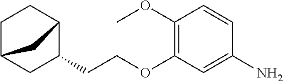 [SMILES: COc1ccc(N)cc1OCC[C@@H]1C[C@@H]2CC[C@H]1C2] |
| 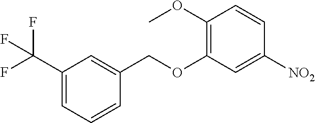 [SMILES: COc1ccc([N+](=O)[O-])cc1OCc1cccc(C(F)(F)F)c1] | 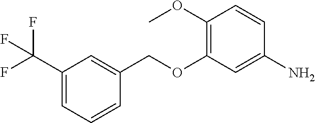 [SMILES: COc1ccc(N)cc1OCc1cccc(C(F)(F)F)c1] |
| 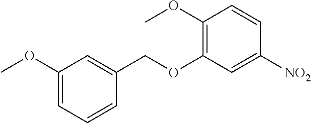 [SMILES: COc1cccc(COc2cc([N+](=O)[O-])ccc2OC)c1] | 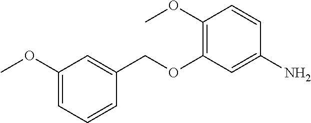 [SMILES: COc1cccc(COc2cc(N)ccc2OC)c1] |
| 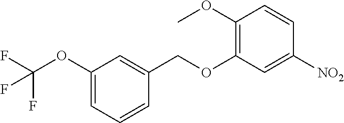 [SMILES: COc1ccc([N+](=O)[O-])cc1OCc1cccc(OC(F)(F)F)c1] | 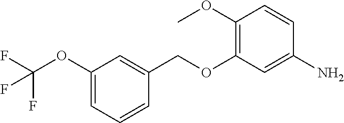 [SMILES: COc1ccc(N)cc1OCc1cccc(OC(F)(F)F)c1] |
| 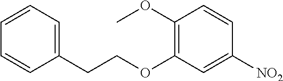 [SMILES: COc1ccc([N+](=O)[O-])cc1OCCc1ccccc1] | 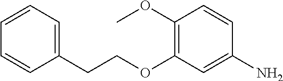 [SMILES: COc1ccc(N)cc1OCCc1ccccc1] |
| 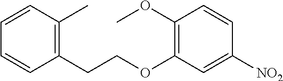 [SMILES: COc1ccc([N+](=O)[O-])cc1OCCc1ccccc1C] | 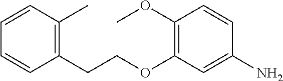 [SMILES: COc1ccc(N)cc1OCCc1ccccc1C] |
| 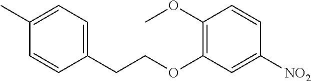 [SMILES: COc1ccc([N+](=O)[O-])cc1OCCc1ccc(C)cc1] | 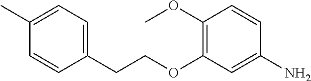 [SMILES: COc1ccc(N)cc1OCCc1ccc(C)cc1] |
| 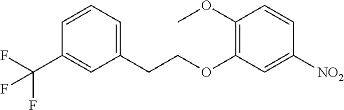 [SMILES: COc1ccc([N+](=O)[O-])cc1OCCc1cccc(C(F)(F)F)c1] | 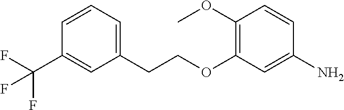 [SMILES: COc1ccc(N)cc1OCCc1cccc(C(F)(F)F)c1] |
| 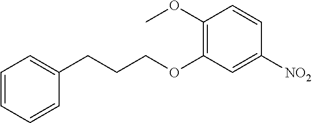 [SMILES: COc1ccc([N+](=O)[O-])cc1OCCCc1ccccc1] | 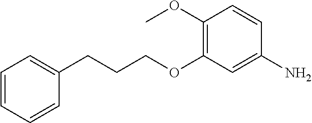 [SMILES: COc1ccc(N)cc1OCCCc1ccccc1] |
| 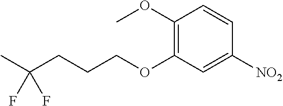 [SMILES: COc1ccc([N+](=O)[O-])cc1OCCCC(C)(F)F] | 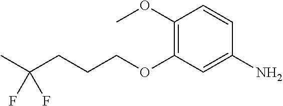 [SMILES: COc1ccc(N)cc1OCCCC(C)(F)F] |
| 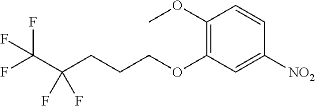 [SMILES: COc1ccc([N+](=O)[O-])cc1OCCCC(F)(F)C(F)(F)F] | 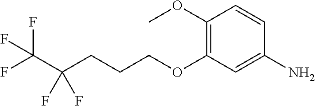 [SMILES: COc1ccc(N)cc1OCCCC(F)(F)C(F)(F)F] |

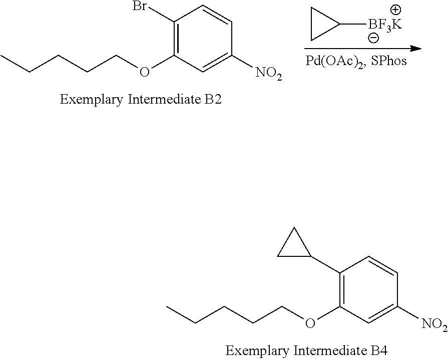

A solution of Exemplary Intermediate B2 (1-bromo-4-nitro-2-(pentyloxy)benzene, 100 mg, 0.26 mmol) in toluene (1.3 mL) and water (0.15 mL) is treated with 3.0 M aqueous potassium phosphate (0.26 mL, 0.77 mmol). The resulting mixture is sparged with argon for 10 minutes. To this mixture is added potassium cyclopropyltrifluoroborate (46 mg, 0.31 mmol), palladium diacetate (6 mg, 0.03 mmol), and SPhos (21 mg, 0.05 mmol). The resulting mixture is sparged with argon an additional 5 minutes. The reaction is sealed under an argon atmosphere then stirred at 100° C. overnight. The reaction is cooled to room temperature then diluted with water (5 mL) and ethyl acetate (5 mL). The layers are separated, and the aqueous phase is extracted with ethyl acetate (3×2 mL). The combined organic layers are dried over sodium sulfate, filtered, and concentrated in vacuo. This material is purified by preparative HPLC (Waters XBridge C18 OBD, 5 μm, 19×150 mm column, 5% to 100% acetonitrile in water with 0.1% formic acid modifier over 45 minutes at 42 mL/min flow) to afford Exemplary Intermediate B4 (1-cyclopropyl-4-nitro-2-(pentyloxy)benzene, 48 mg, 75% yield).

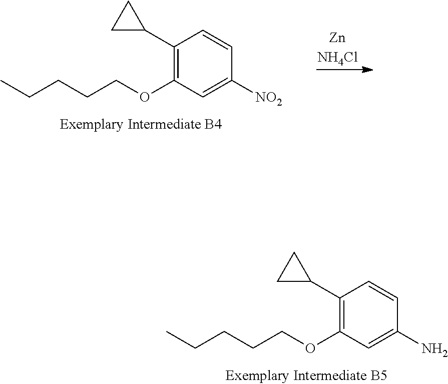 [SMILES: CCCCCOc1cc([N+](=O)[O-])ccc1C1CC1 ; CCCCCOc1cc(N)ccc1C1CC1]

A solution of Exemplary Intermediate B4 (1-cyclopropyl-4-nitro-2-(pentyloxy)benzene, 48 mg, 0.19 mmol) in acetone (0.6 mL) is treated with saturated aqueous ammonium chloride solution (275 μL, 1.93 mmol) and zinc powder (76 mg, 1.16 mmol). The resulting suspension is stirred at room temperature overnight. The reaction is diluted with acetone (10 mL) then filtered through Celite with additional acetone. The filtered solution is concentrated in vacuo. This material is taken up in dichloromethane (3 mL) and washed with saturated aqueous sodium bicarbonate solution (2 mL). The aqueous phase is extracted with dichloromethane (2×3 mL). The combined organic layers are dried over sodium sulfate, filtered, and concentrated in vacuo to afford Exemplary Intermediate B5 (4-cyclopropyl-3-(pentyloxy)aniline, 43 mg).

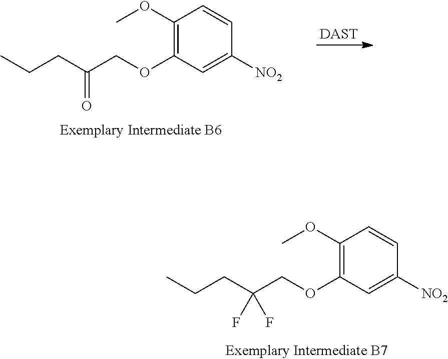 [SMILES: CCCC(=O)COc1cc([N+](=O)[O-])ccc1OC ; "DAST" ; CCCC(F)(F)COc1cc([N+](=O)[O-])ccc1OC]

A mixture of Exemplary Intermediate B6 (1-(2-methoxy-5-nitrophenoxy)pentan-2-one, 4.7 g, 18.7 mmol) and DAST (5 mL) is stirred at room temperature overnight then quenched with ice-water (200 mL) carefully. The resulting mixture is extracted with ethyl acetate (3×150 mL). The combined organic phases are washed with brine (3×200 mL), dried over anhydrous sodium sulfate, filtered and concentrated in vacuo. The residue is suspended with hexane-ethyl acetate (10:1 v/v, 50 mL) and filtered. The cake is dried to afford Exemplary Intermediate B7 (2-(2,2-difluoropentyloxy)-1-methoxy-4-nitrobenzene, 5.56 g, >99% yield).

The following compounds are prepared in a similar manner as Exemplary Intermediate B5, as described above.

| Ketone | Compound |
|---|---|
| 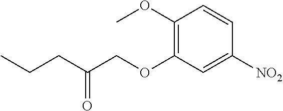 [SMILES: CCCC(=O)COc1cc([N+](=O)[O-])ccc1OC] | 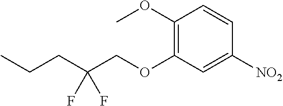 [SMILES: CCCC(F)(F)COc1cc([N+](=O)[O-])ccc1OC] |
| Exemplary Intermediate B6 | Exemplary Intermediate B7 |
| 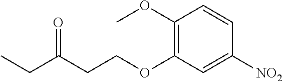 [SMILES: CCC(=O)CCOc1cc([N+](=O)[O-])ccc1OC] | 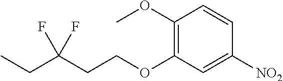 [SMILES: CCC(F)(F)CCOc1cc([N+](=O)[O-])ccc1OC] |

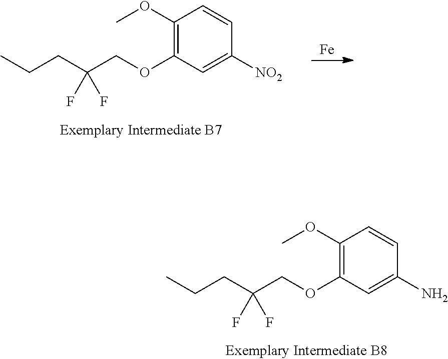

Iron powder (5.2 g, 93.3 mmol) and ammonium chloride (8.0 g, 93.3 mmol) are added to a solution of Exemplary Intermediate B7 (2-(2,2-difluoropentyloxy)-1-methoxy-4-nitrobenzene, 5.1 g, 18.7 mmol) in a mixture of ethanol (30 mL) and water (6 mL). The mixture is heated at 80° C. for 12 h then cooled to room temperature and diluted with additional water (300 mL) and ethyl acetate (300 mL). The mixture is filtered, the phases of the filtered solution are separated. The organic phase is washed with brine (3×300 mL), dried over anhydrous sodium sulfate, filtered, and concentrated in vacuo to about 50 mL. 8.0 M hydrogen chloride in 1,4-dioxane (3 mL) is added to produce a precipitate. The mixture is stirred for 0.5 h, then filtered. The collected solids are washed with ethyl acetate (2×10 mL) then hexane (2×10 mL) and dried under vacuum to afford Exemplary Intermediate B8 (3-((2,2-difluoropentyl)oxy)-4-methoxyaniline as an HCl salt, 3.3 g, 62% yield).

The following compounds are prepared in a similar manner as Exemplary Intermediate B8, as described above.

| Nitro | Compound |
|---|---|
| 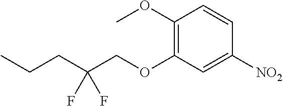 [SMILES: CCCC(F)(F)COc1cc([N+](=O)[O-])ccc1OC] | 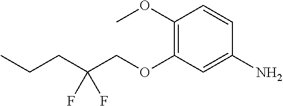 [SMILES: CCCC(F)(F)COc1cc(N)ccc1OC] |
| Exemplary Intermediate B7 | |
| 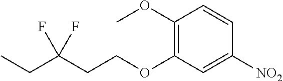 [SMILES: CCC(F)(F)CCOc1cc([N+](=O)[O-])ccc1OC] | 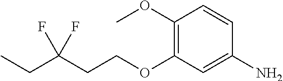 [SMILES: CCC(F)(F)CCOc1cc(N)ccc1OC] |

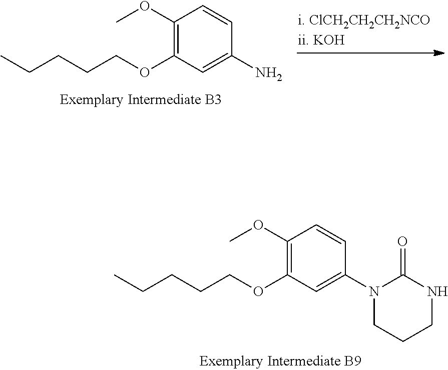 [SMILES: CCCCCOc1cc(N)ccc1OC ; CCCCCOc1cc(N2CCCNC2=O)ccc1OC]

To a solution of Exemplary Intermediate B3 (4-methoxy-3 pentyloxy)aniline, 10.0 g, 47.8 mmol) in tetrahydrofuran (100 mL) is added 3-chloropropyl isocyanate (5.6 g 52.6 mmol) at room temperature. The mixture is stirred overnight at room temperature. Powdered potassium hydroxide (4.0 g, 71.8 mmol) is added to the reaction mixture, the resulting mixture is stirred at 50° C. overnight. The reaction is diluted with water (300 mL), and the resulting precipitated is collected by filtration. The solids are washed with ethyl acetate and dried under vacuum to afford Exemplary Intermediate B9 (1-(4-methoxy-3-(pentyloxy)phenyl)tetrahydropyrimidin-2(1H)-one, 5.3 g, 40% yield).

The following compounds are prepared in a similar manner as Exemplary Intermediate B9 as described above.

| Aniline | Isocyanate | Compound |
|---|---|---|
| 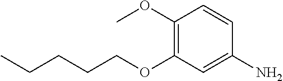 Exemplary Intermediate B3 | 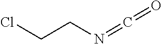 | 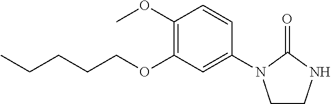 [SMILES: CCCCCOc1cc(N2CCNC2=O)ccc1OC] |
| 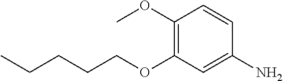 | 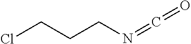 | 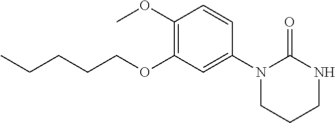 [SMILES: CCCCCOc1cc(N2CCCNC2=O)ccc1OC] |
| Exemplary Intermediate B3 | | Exemplary Intermediate B9 |
| 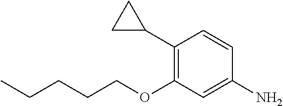 [SMILES: CCCCCOc1cc(N)ccc1C1CC1] | 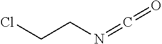 | 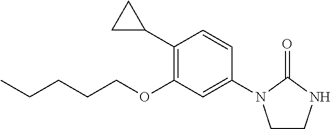 [SMILES: CCCCCOc1cc(N2CCNC2=O)ccc1C1CC1] |
| Exemplary Intermediate B5 | | |
| 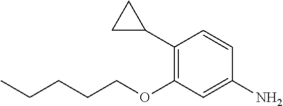 [SMILES: CCCCCOc1cc(N)ccc1C1CC1] Exemplary Intermediate B5 | 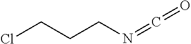 | 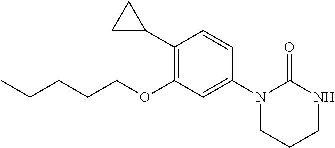 [SMILES: CCCCCOc1cc(N2CCCNC2=O)ccc1C1CC1] |
| 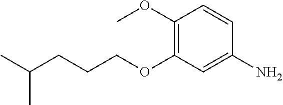 [SMILES: COc1ccc(N)cc1OCCCC(C)C] | 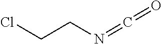 | 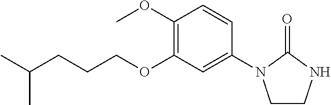 [SMILES: COc1ccc(N2CCNC2=O)cc1OCCCC(C)C] |
| 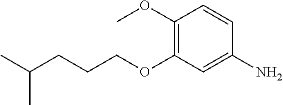 [SMILES: COc1ccc(N)cc1OCCCC(C)C] | 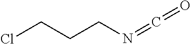 | 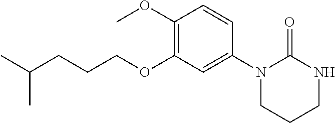 [SMILES: COc1ccc(N2CCCNC2=O)cc1OCCCC(C)C] |
| 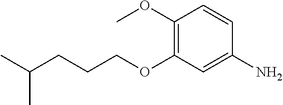 [SMILES: COc1ccc(N)cc1OCCCC(C)C] | 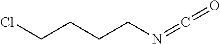 | 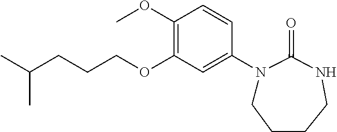 [SMILES: COc1ccc(N2CCCCNC2=O)cc1OCCCC(C)C] |
| 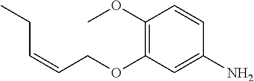 | 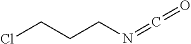 | 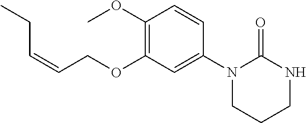 [SMILES: CC/C=C\COc1cc(N2CCCNC2=O)ccc1OC] |
| 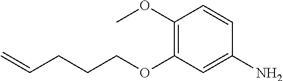 | 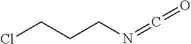 | 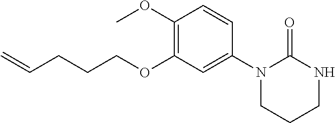 [SMILES: C=CCCCOc1cc(N2CCCNC2=O)ccc1OC] |
| 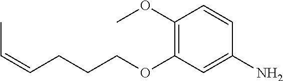 [SMILES: C/C=C\CCCOc1cc(N)ccc1OC] | 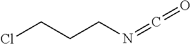 | 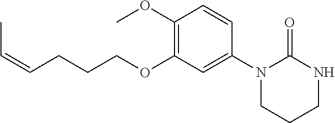 [SMILES: C/C=C\CCCOc1cc(N2CCCNC2=O)ccc1OC] |
| 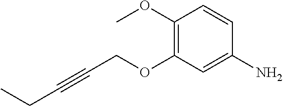 | 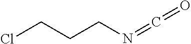 | 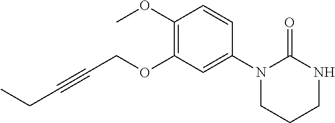 [SMILES: CCC#CCOc1cc(N2CCCNC2=O)ccc1OC] |
| 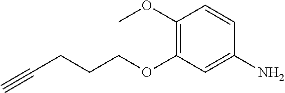 | 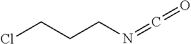 | 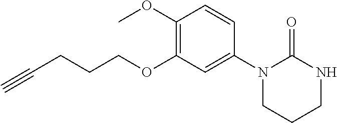 [SMILES: C#CCCCOc1cc(N2CCCNC2=O)ccc1OC] |
| 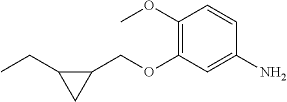 [SMILES: CCC1CC1COc1cc(N)ccc1OC] | 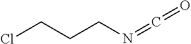 | 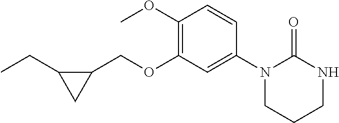 [SMILES: CCC1CC1COc1cc(N2CCCNC2=O)ccc1OC] |
| 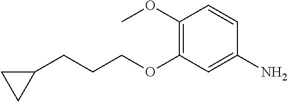 [SMILES: COc1ccc(N)cc1OCCCC1CC1] | 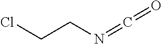 | 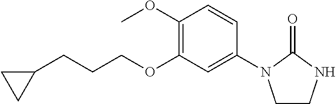 [SMILES: COc1ccc(N2CCNC2=O)cc1OCCCC1CC1] |
| 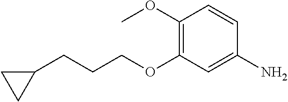 [SMILES: COc1ccc(N)cc1OCCCC1CC1] | 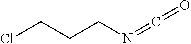 | 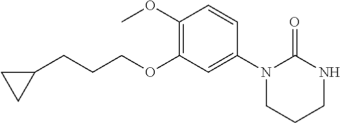 [SMILES: COc1ccc(N2CCCNC2=O)cc1OCCCC1CC1] |
| 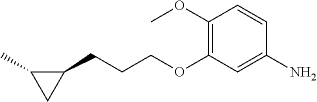 [SMILES: COc1ccc(N)cc1OCCC[C@H]1C[C@@H]1C] | 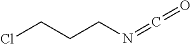 | 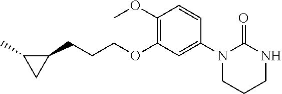 [SMILES: COc1ccc(N2CCCNC2=O)cc1OCCC[C@H]1C[C@@H]1C] |
| | | Racemic |
| 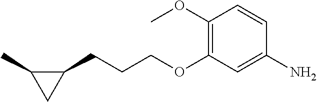 [SMILES: COc1ccc(N)cc1OCCC[C@H]1C[C@H]1C] | 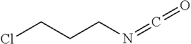 | 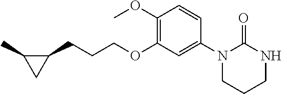 [SMILES: COc1ccc(N2CCCNC2=O)cc1OCCC[C@H]1C[C@H]1C] |
| | | Racemic |
| 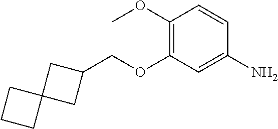 [SMILES: COc1ccc(N)cc1OCC1CC2(CCC2)C1] | 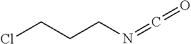 | 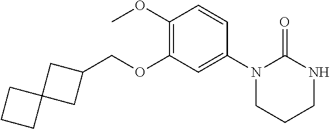 [SMILES: COc1ccc(N2CCCNC2=O)cc1OCC1CC2(CCC2)C1] |
| 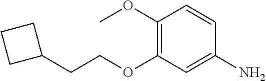 [SMILES: COc1ccc(N)cc1OCCC1CCC1] | 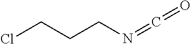 | 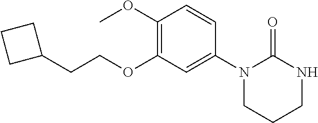 [SMILES: COc1ccc(N2CCCNC2=O)cc1OCCC1CCC1] |
| 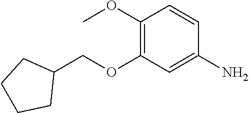 [SMILES: COc1ccc(N)cc1OCC1CCCC1] | 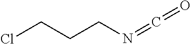 | 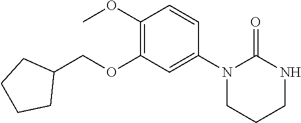 [SMILES: COc1ccc(N2CCCNC2=O)cc1OCC1CCCC1] |
| 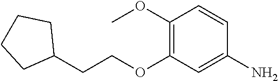 [SMILES: COc1ccc(N)cc1OCCC1CCCC1] | 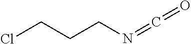 | 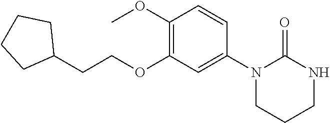 [SMILES: COc1ccc(N2CCCNC2=O)cc1OCCC1CCCC1] |
| 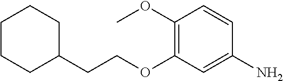 [SMILES: COc1ccc(N)cc1OCCC1CCCCC1] | 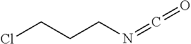 | 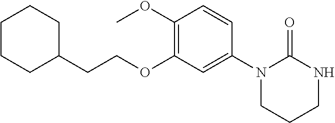 [SMILES: COc1ccc(N2CCCNC2=O)cc1OCCC1CCCCC1] |
| 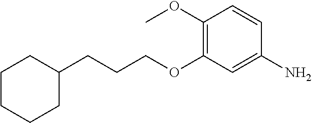 [SMILES: COc1ccc(N)cc1OCCCC1CCCCC1] | 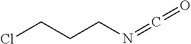 | 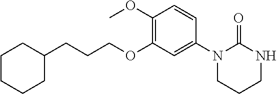 [SMILES: COc1ccc(N2CCCNC2=O)cc1OCCCC1CCCCC1] |
| 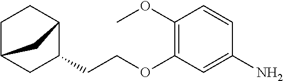 [SMILES: COc1ccc(N)cc1OCC[C@@H]1C[C@@H]2CC[C@H]1C2] | 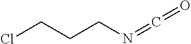 | 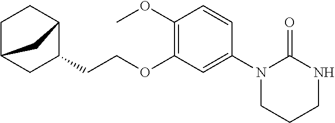 [SMILES: COc1ccc(N2CCCNC2=O)cc1OCC[C@@H]1C[C@@H]2CC[C@H]1C2] |
| 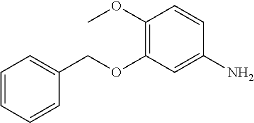 [SMILES: COc1ccc(N)cc1OCc1ccccc1] | 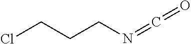 | 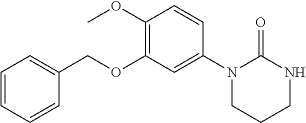 [SMILES: COc1ccc(N2CCCNC2=O)cc1OCc1ccccc1] |
| 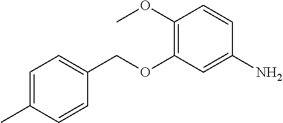 [SMILES: COc1ccc(N)cc1OCc1ccc(C)cc1] | 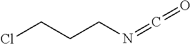 | 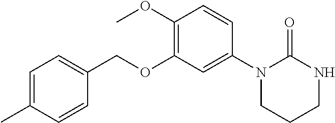 [SMILES: COc1ccc(N2CCCNC2=O)cc1OCc1ccc(C)cc1] |
| 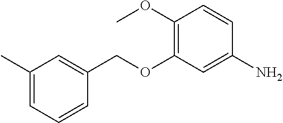 [SMILES: COc1ccc(N)cc1OCc1cccc(C)c1] | 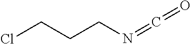 | 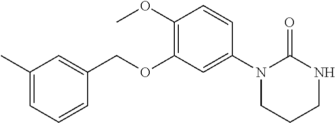 [SMILES: COc1ccc(N2CCCNC2=O)cc1OCc1cccc(C)c1] |
| 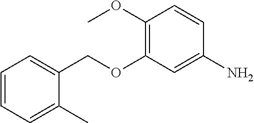 [SMILES: COc1ccc(N)cc1OCc1ccccc1C] | 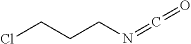 | 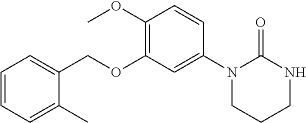 [SMILES: COc1ccc(N2CCCNC2=O)cc1OCc1ccccc1C] |
| 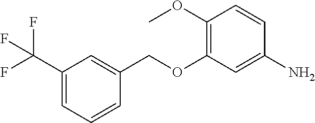 [SMILES: COc1ccc(N)cc1OCc1cccc(C(F)(F)F)c1] | 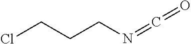 | 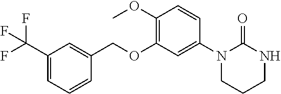 [SMILES: COc1ccc(N2CCCNC2=O)cc1OCc1cccc(C(F)(F)F)c1] |
| 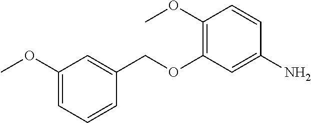 [SMILES: COc1cccc(COc2cc(N)ccc2OC)c1] | 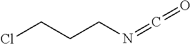 | 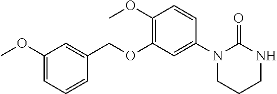 [SMILES: COc1cccc(COc2cc(N3CCCNC3=O)ccc2OC)c1] |
| 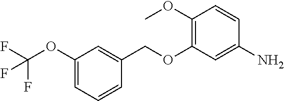 [SMILES: COc1ccc(N)cc1OCc1cccc(OC(F)(F)F)c1] | 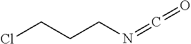 | 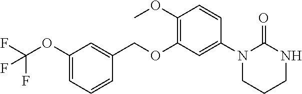 [SMILES: COc1ccc(N2CCCNC2=O)cc1OCc1cccc(OC(F)(F)F)c1] |
| 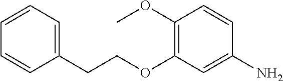 [SMILES: COc1ccc(N)cc1OCCc1ccccc1] | 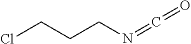 | 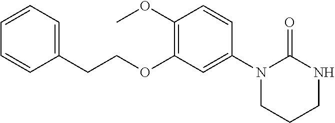 [SMILES: COc1ccc(N2CCCNC2=O)cc1OCCc1ccccc1] |
| 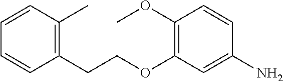 [SMILES: COc1ccc(N)cc1OCCc1ccccc1C] | 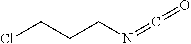 | 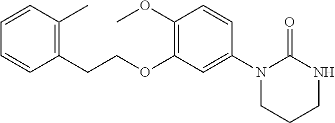 [SMILES: COc1ccc(N2CCCNC2=O)cc1OCCc1ccccc1C] |
| 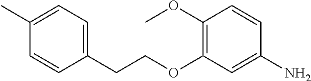 [SMILES: COc1ccc(N)cc1OCCc1ccc(C)cc1] | 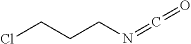 | 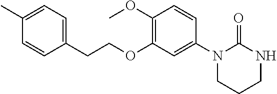 [SMILES: COc1ccc(N2CCCNC2=O)cc1OCCc1ccc(C)cc1] |
| 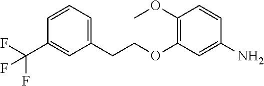 [SMILES: COc1ccc(N)cc1OCCc1cccc(C(F)(F)F)c1] | 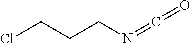 | 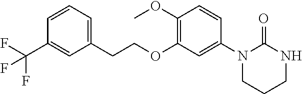 [SMILES: COc1ccc(N2CCCNC2=O)cc1OCCc1cccc(C(F)(F)F)c1] |
| 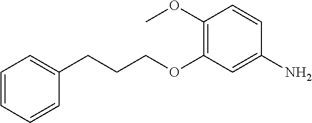 [SMILES: COc1ccc(N)cc1OCCCc1ccccc1] | 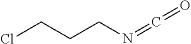 | 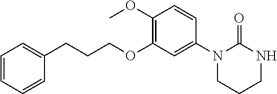 [SMILES: COc1ccc(N2CCCNC2=O)cc1OCCCc1ccccc1] |
| 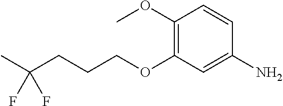 [SMILES: COc1ccc(N)cc1OCCCC(C)(F)F] | 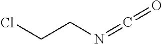 | 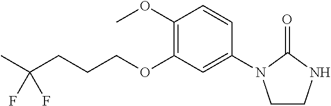 [SMILES: COc1ccc(N2CCNC2=O)cc1OCCCC(C)(F)F] |
| 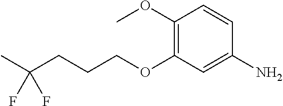 [SMILES: COc1ccc(N)cc1OCCCC(C)(F)F] | 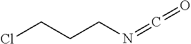 | 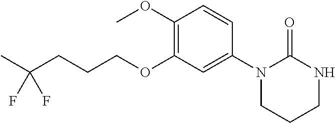 [SMILES: COc1ccc(N2CCCNC2=O)cc1OCCCC(C)(F)F] |
| 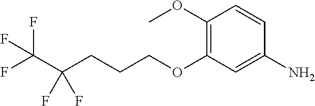 [SMILES: COc1ccc(N)cc1OCCCC(F)(F)C(F)(F)F] | 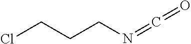 | 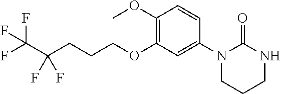 [SMILES: COc1ccc(N2CCCNC2=O)cc1OCCCC(F)(F)C(F)(F)F] |
| 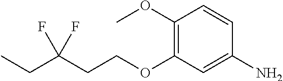 [SMILES: CCC(F)(F)CCOc1cc(N)ccc1OC] | 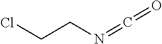 | 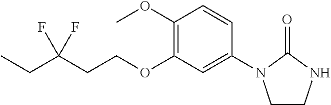 [SMILES: CCC(F)(F)CCOc1cc(N2CCNC2=O)ccc1OC] |
| 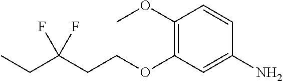 [SMILES: CCC(F)(F)CCOc1cc(N)ccc1OC] | 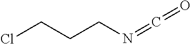 | 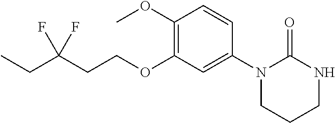 [SMILES: CCC(F)(F)CCOc1cc(N2CCCNC2=O)ccc1OC] |
| 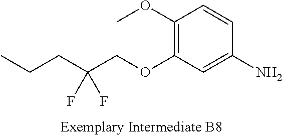 [SMILES: CCCC(F)(F)COc1cc(N)ccc1OC] | 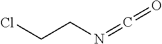 | 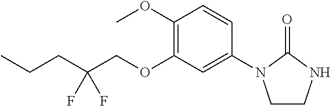 [SMILES: CCCC(F)(F)COc1cc(N2CCNC2=O)ccc1OC] |
| 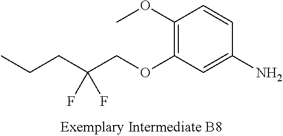 [SMILES: CCCC(F)(F)COc1cc(N)ccc1OC] | 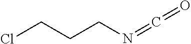 | 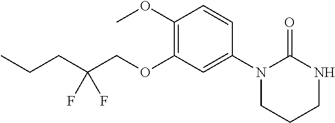 [SMILES: CCCC(F)(F)COc1cc(N2CCCNC2=O)ccc1OC] |
| 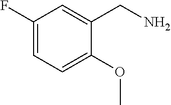 | 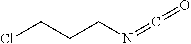 | 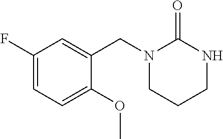 [SMILES: COc1ccc(F)cc1CN1CCCNC1=O] |
| 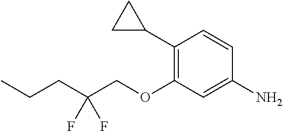 [SMILES: CCCC(F)(F)COc1cc(N)ccc1C1CC1] | 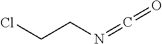 | 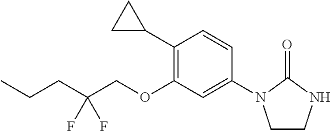 [SMILES: CCCC(F)(F)COc1cc(N2CCNC2=O)ccc1C1CC1] |
| 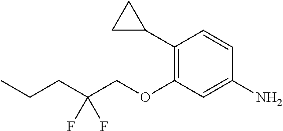 [SMILES: CCCC(F)(F)COc1cc(N)ccc1C1CC1] | 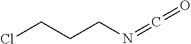 | 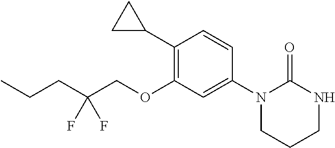 [SMILES: CCCC(F)(F)COc1cc(N2CCCNC2=O)ccc1C1CC1] |

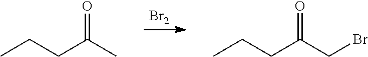

Bromine (27.9 g, 0.174 mol) is added dropwise to a solution of pentan-2-one (15.0 g, 0.17 mol) in methanol (150 mL) at 0° C. over 0.5 h. The mixture is warmed to room temperature and stirred overnight. The mixture is concentrated in vacuo. The residue is dissolved in dichloromethane (200 mL), washed with brine (3×100 mL), dried over anhydrous sodium sulfate, filtered, and concentrated in vacuo to afford 1-bromopentan-2-one (13.9 g).

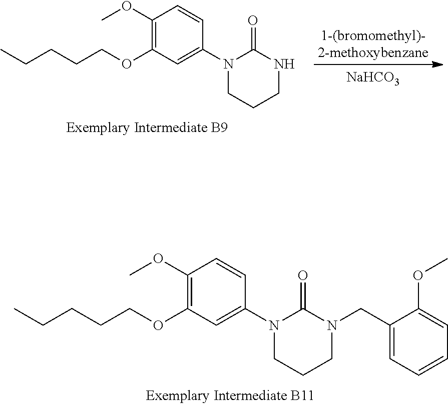

Exemplary Intermediate B9 (4-methoxy-3-(pentyl oxy)aniline) is combined with 1-(bromomethyl)-2-methoxybenzene and sodium bicarbonate. The resulting reaction affords Exemplary Intermediate B11 (1-(4-methoxy-3-(pentyloxy)phenyl)tetrahydropyrimidin-2(1H)-one, 5.3 g, 40% yield).

The following compounds are prepared in a similar manner as Exemplary Intermediate B11, as described above.

| Cyclic Urea | Bromide | Compound |
|---|---|---|
| 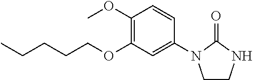 [SMILES: CCCCCOc1cc(N2CCNC2=O)ccc1OC] | 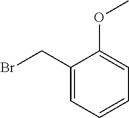 | 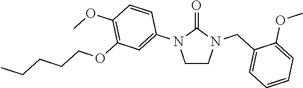 [SMILES: CCCCCOc1cc(N2CCN(Cc3ccccc3OC)C2=O)ccc1OC] |
| 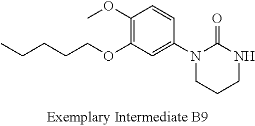 [SMILES: CCCCCOc1cc(N2CCCNC2=O)ccc1OC] | 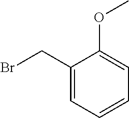 | 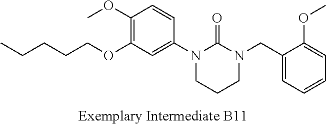 [SMILES: CCCCCOc1cc(N2CCCN(Cc3ccccc3OC)C2=O)ccc1OC] |
| 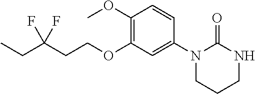 [SMILES: CCC(F)(F)CCOc1cc(N2CCCNC2=O)ccc1OC] | 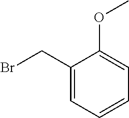 | 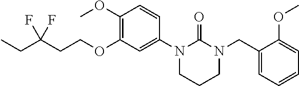 [SMILES: CCC(F)(F)CCOc1cc(N2CCCN(Cc3ccccc3OC)C2=O)ccc1OC] |
| 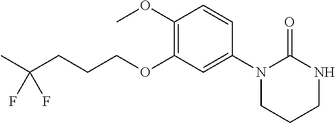 [SMILES: COc1ccc(N2CCCNC2=O)cc1OCCCC(C)(F)F] | 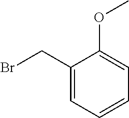 | 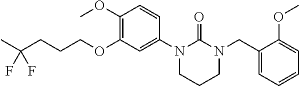 [SMILES: COc1ccccc1CN1CCCN(c2ccc(OC)c(OCCCC(C)(F)F)c2)C1=O] |
| 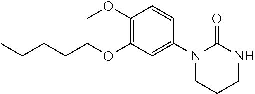 [SMILES: CCCCCOc1cc(N2CCCNC2=O)ccc1OC] | 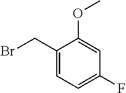 | 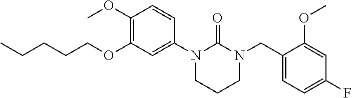 [SMILES: CCCCCOc1cc(N2CCCN(Cc3ccc(F)cc3OC)C2=O)ccc1OC] |
| 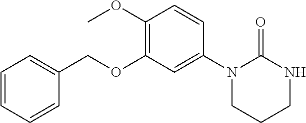 [SMILES: COc1ccc(N2CCCNC2=O)cc1OCc1ccccc1] | 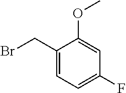 | 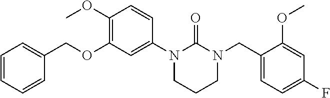 [SMILES: COc1cc(F)ccc1CN1CCCN(c2ccc(OC)c(OCc3ccccc3)c2)C1=O] |
| 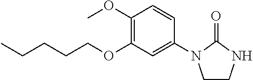 [SMILES: CCCCCOc1cc(N2CCNC2=O)ccc1OC] | 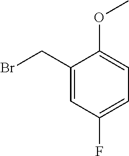 | 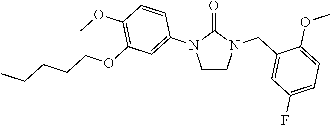 [SMILES: CCCCCOc1cc(N2CCN(Cc3cc(F)ccc3OC)C2=O)ccc1OC] |
| 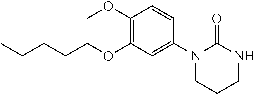 [SMILES: CCCCCOc1cc(N2CCCNC2=O)ccc1OC] | 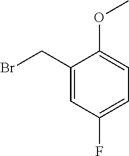 | 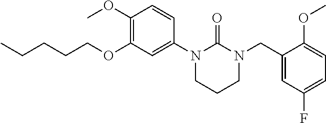 [SMILES: CCCCCOc1cc(N2CCCN(Cc3cc(F)ccc3OC)C2=O)ccc1OC] |
| 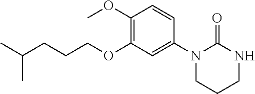 [SMILES: COc1ccc(N2CCCNC2=O)cc1OCCCC(C)C] | 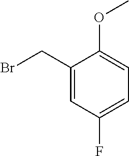 | 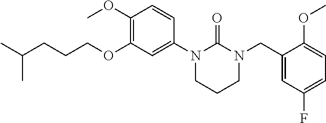 [SMILES: COc1ccc(F)cc1CN1CCCN(c2ccc(OC)c(OCCCC(C)C)c2)C1=O] |
| 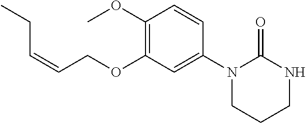 [SMILES: CC/C=C\COc1cc(N2CCCNC2=O)ccc1OC] | 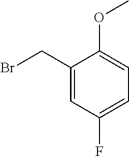 | 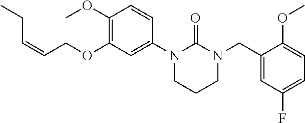 [SMILES: CC/C=C\COc1cc(N2CCCN(Cc3cc(F)ccc3OC)C2=O)ccc1OC] |
| 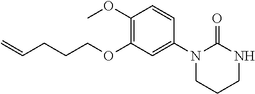 [SMILES: C=CCCCOc1cc(N2CCCNC2=O)ccc1OC] | 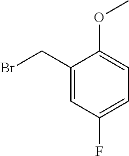 | 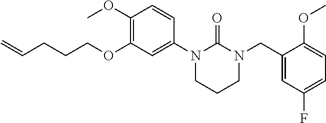 [SMILES: C=CCCCOc1cc(N2CCCN(Cc3cc(F)ccc3OC)C2=O)ccc1OC] |
| 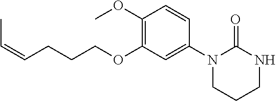 [SMILES: C/C=C\CCCOc1cc(N2CCCNC2=O)ccc1OC] | 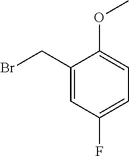 | 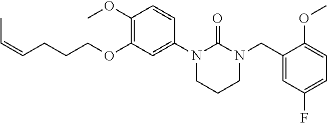 [SMILES: C/C=C\CCCOc1cc(N2CCCN(Cc3cc(F)ccc3OC)C2=O)ccc1OC] |
| 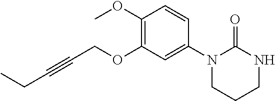 [SMILES: CCC#CCOc1cc(N2CCCNC2=O)ccc1OC] | 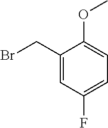 | 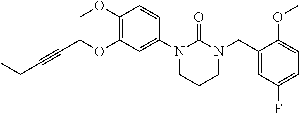 [SMILES: CCC#CCOc1cc(N2CCCN(Cc3cc(F)ccc3OC)C2=O)ccc1OC] |
| 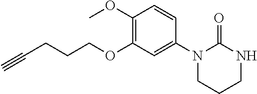 [SMILES: C#CCCCOc1cc(N2CCCNC2=O)ccc1OC] | 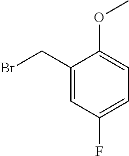 | 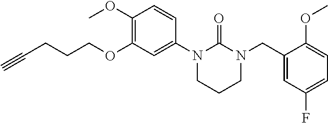 [SMILES: C#CCCCOc1cc(N2CCCN(Cc3cc(F)ccc3OC)C2=O)ccc1OC] |
| 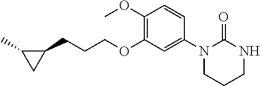 [SMILES: COc1ccc(N2CCCNC2=O)cc1OCCC[C@H]1C[C@@H]1C] | 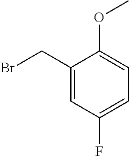 | 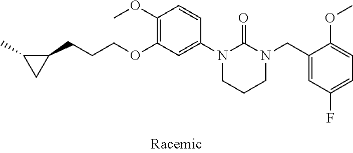 |
| 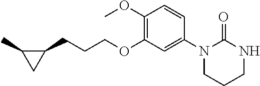 [SMILES: COc1ccc(N2CCCNC2=O)cc1OCCC[C@H]1C[C@H]1C] | 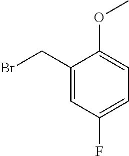 | 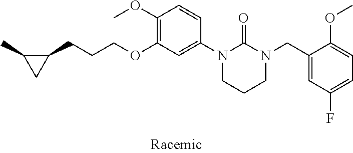 |
| 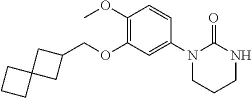 [SMILES: COc1ccc(N2CCCNC2=O)cc1OCC1CC2(CCC2)C1] | 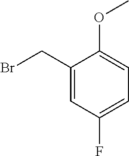 | 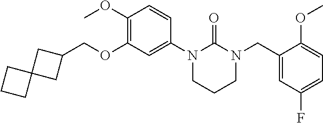 [SMILES: COc1ccc(F)cc1CN1CCCN(c2ccc(OC)c(OCC3CC4(CCC4)C3)c2)C1=O] |
| 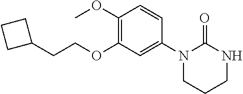 [SMILES: COc1ccc(N2CCCNC2=O)cc1OCCC1CCC1] | 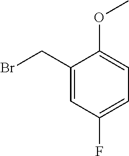 | 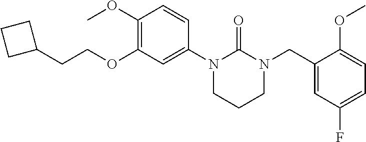 [SMILES: COc1ccc(F)cc1CN1CCCN(c2ccc(OC)c(OCCC3CCC3)c2)C1=O] |
| 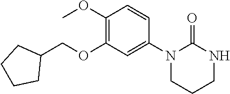 [SMILES: COc1ccc(N2CCCNC2=O)cc1OCC1CCCC1] | 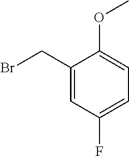 | 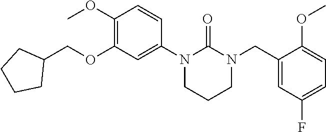 [SMILES: COc1ccc(F)cc1CN1CCCN(c2ccc(OC)c(OCC3CCCC3)c2)C1=O] |
| 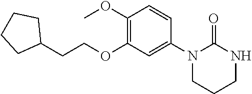 [SMILES: COc1ccc(N2CCCNC2=O)cc1OCCC1CCCC1] | 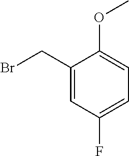 | 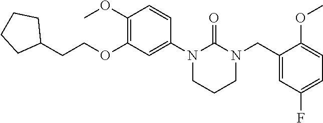 [SMILES: COc1ccc(F)cc1CN1CCCN(c2ccc(OC)c(OCCC3CCCC3)c2)C1=O] |
| 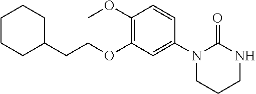 [SMILES: COc1ccc(N2CCCNC2=O)cc1OCCC1CCCCC1] | 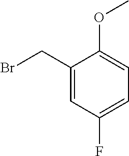 | 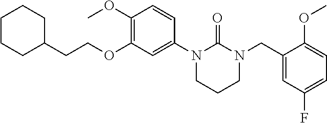 [SMILES: COc1ccc(F)cc1CN1CCCN(c2ccc(OC)c(OCCC3CCCCC3)c2)C1=O] |
| 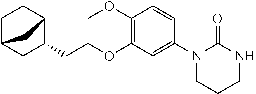 [SMILES: COc1ccc(N2CCCNC2=O)cc1OCC[C@@H]1C[C@@H]2CC[C@H]1C2] | 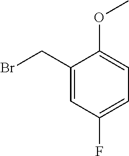 | 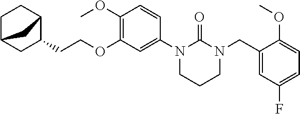 [SMILES: COc1ccc(F)cc1CN1CCCN(c2ccc(OC)c(OCC[C@@H]3C[C@@H]4CC[C@H]3C4)c2)C1=O] |
| 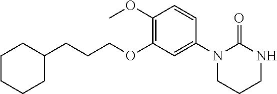 [SMILES: COc1ccc(N2CCCNC2=O)cc1OCCCC1CCCCC1] | 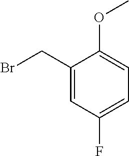 | 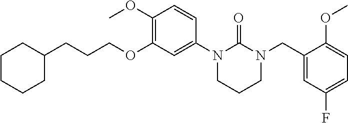 [SMILES: COc1ccc(F)cc1CN1CCCN(c2ccc(OC)c(OCCCC3CCCCC3)c2)C1=O] |
| 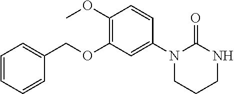 [SMILES: COc1ccc(N2CCCNC2=O)cc1OCc1ccccc1] | 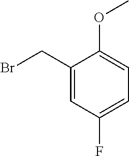 | 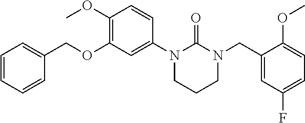 [SMILES: COc1ccc(F)cc1CN1CCCN(c2ccc(OC)c(OCc3ccccc3)c2)C1=O] |
| 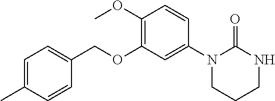 [SMILES: COc1ccc(N2CCCNC2=O)cc1OCc1ccc(C)cc1] | 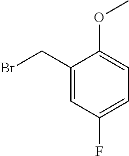 | 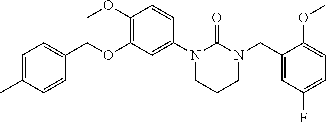 |
| 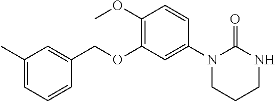 [SMILES: COc1ccc(N2CCCNC2=O)cc1OCc1cccc(C)c1] | 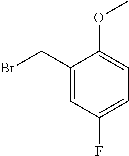 | 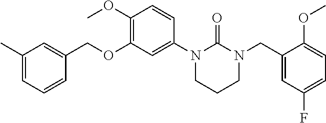 |
| 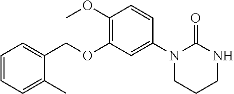 [SMILES: COc1ccc(N2CCCNC2=O)cc1OCc1ccccc1C] | 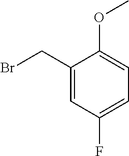 | 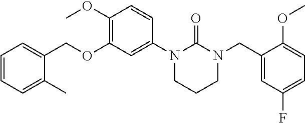 [SMILES: COc1ccc(F)cc1CN1CCCN(c2ccc(OC)c(OCc3ccccc3C)c2)C1=O] |
| 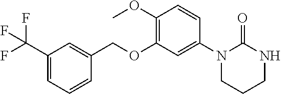 [SMILES: COc1ccc(N2CCCNC2=O)cc1OCc1cccc(C(F)(F)F)c1] | 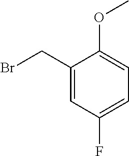 | 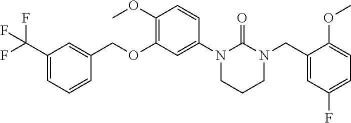 [SMILES: COc1ccc(F)cc1CN1CCCN(c2ccc(OC)c(OCc3cccc(C(F)(F)F)c3)c2)C1=O] |
| 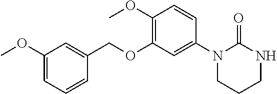 [SMILES: COc1cccc(COc2cc(N3CCCNC3=O)ccc2OC)c1] | 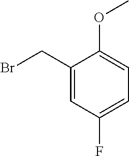 | 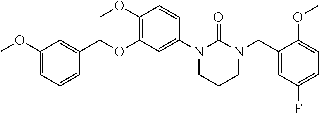 |
| 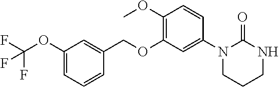 [SMILES: COc1ccc(N2CCCNC2=O)cc1OCc1cccc(OC(F)(F)F)c1] | 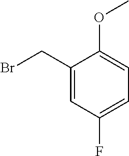 | 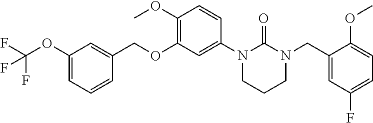 [SMILES: COc1ccc(F)cc1CN1CCCN(c2ccc(OC)c(OCc3cccc(OC(F)(F)F)c3)c2)C1=O] |
| 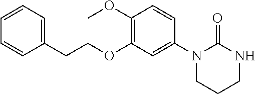 [SMILES: COc1ccc(N2CCCNC2=O)cc1OCCc1ccccc1] | 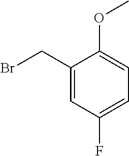 | 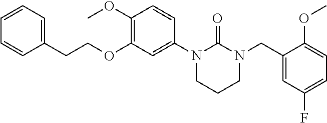 [SMILES: COc1ccc(F)cc1CN1CCCN(c2ccc(OC)c(OCCc3ccccc3)c2)C1=O] |
| 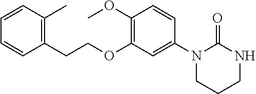 [SMILES: COc1ccc(N2CCCNC2=O)cc1OCCc1ccccc1C] | 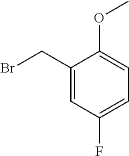 | 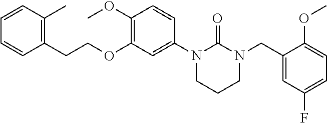 [SMILES: COc1ccc(F)cc1CN1CCCN(c2ccc(OC)c(OCCc3ccccc3C)c2)C1=O] |
| 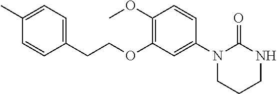 [SMILES: COc1ccc(N2CCCNC2=O)cc1OCCc1ccc(C)cc1] | 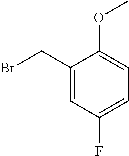 | 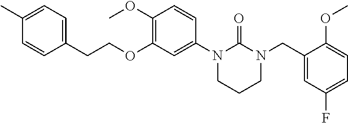 |
| 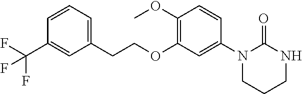 [SMILES: COc1ccc(N2CCCNC2=O)cc1OCCc1cccc(C(F)(F)F)c1] | 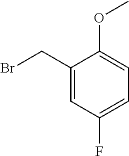 | 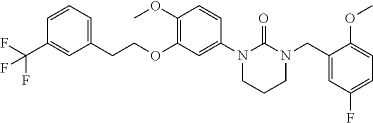 [SMILES: COc1ccc(F)cc1CN1CCCN(c2ccc(OC)c(OCCc3cccc(C(F)(F)F)c3)c2)C1=O] |
| 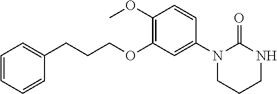 [SMILES: COc1ccc(N2CCCNC2=O)cc1OCCCc1ccccc1] | 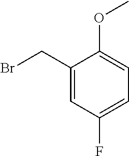 | 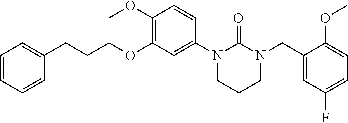 [SMILES: COc1ccc(F)cc1CN1CCCN(c2ccc(OC)c(OCCCc3ccccc3)c2)C1=O] |
| 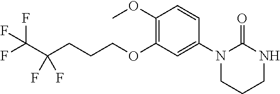 [SMILES: COc1ccc(N2CCCNC2=O)cc1OCCCC(F)(F)C(F)(F)F] | 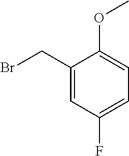 | 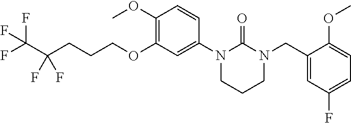 [SMILES: COc1ccc(F)cc1CN1CCCN(c2ccc(OC)c(OCCCC(F)(F)C(F)(F)F)c2)C1=O] |
| 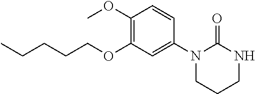 [SMILES: CCCCCOc1cc(N2CCCNC2=O)ccc1OC] | 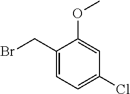 | 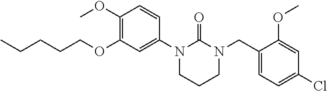 [SMILES: CCCCCOc1cc(N2CCCN(Cc3ccc(Cl)cc3OC)C2=O)ccc1OC] |
| 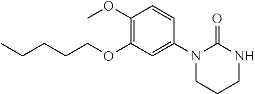 [SMILES: CCCCCOc1cc(N2CCCNC2=O)ccc1OC] | 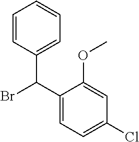 [SMILES: COc1cc(Cl)ccc1C(Br)c1ccccc1] | 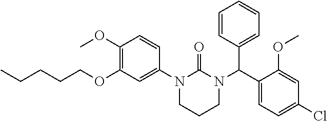 [SMILES: CCCCCOc1cc(N2CCCN(C(c3ccccc3)c3ccc(Cl)cc3OC)C2=O)ccc1OC] |
| 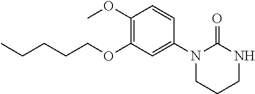 [SMILES: CCCCCOc1cc(N2CCCNC2=O)ccc1OC] | 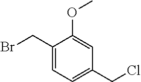 | 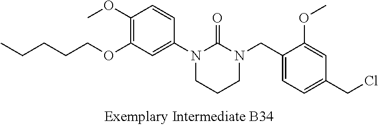 [SMILES: CCCCCOc1cc(N2CCCN(Cc3ccc(CCl)cc3OC)C2=O)ccc1OC] |
| 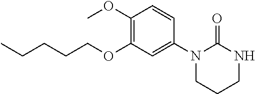 [SMILES: CCCCCOc1cc(N2CCCNC2=O)ccc1OC] | 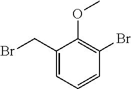 | 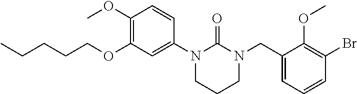 [SMILES: CCCCCOc1cc(N2CCCN(Cc3cccc(Br)c3OC)C2=O)ccc1OC] |
| 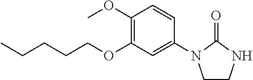 [SMILES: CCCCCOc1cc(N2CCNC2=O)ccc1OC] | 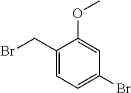 | 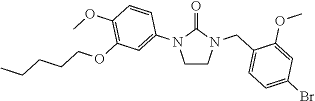 [SMILES: CCCCCOc1cc(N2CCN(Cc3ccc(Br)cc3OC)C2=O)ccc1OC] |
| 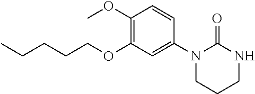 [SMILES: CCCCCOc1cc(N2CCCNC2=O)ccc1OC] | 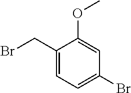 | 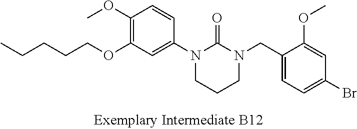 [SMILES: CCCCCOc1cc(N2CCCN(Cc3ccc(Br)cc3OC)C2=O)ccc1OC] |
| 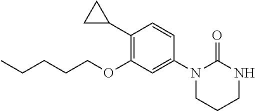 [SMILES: CCCCCOc1cc(N2CCCNC2=O)ccc1C1CC1] | 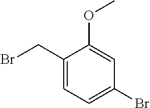 | 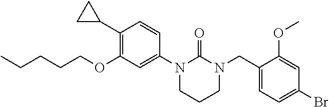 [SMILES: CCCCCOc1cc(N2CCCN(Cc3ccc(Br)cc3OC)C2=O)ccc1C1CC1] |
| 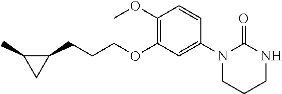 [SMILES: COc1ccc(N2CCCNC2=O)cc1OCCC[C@H]1C[C@H]1C] | 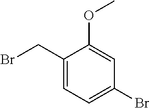 | 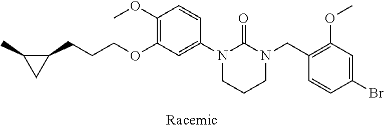 [SMILES: COc1cc(Br)ccc1CN1CCCN(c2ccc(OC)c(OCCC[C@H]3C[C@H]3C)c2)C1=O] |
| 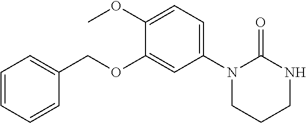 [SMILES: COc1ccc(N2CCCNC2=O)cc1OCc1ccccc1] | 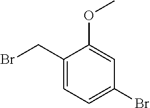 | 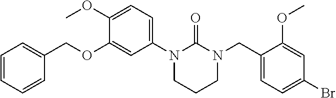 [SMILES: COc1cc(Br)ccc1CN1CCCN(c2ccc(OC)c(OCc3ccccc3)c2)C1=O] |
| 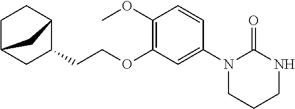 [SMILES: COc1ccc(N2CCCNC2=O)cc1OCC[C@@H]1C[C@@H]2CC[C@H]1C2] | 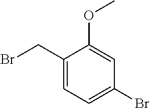 | 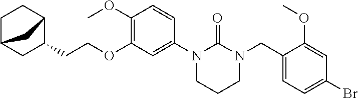 [SMILES: COc1cc(Br)ccc1CN1CCCN(c2ccc(OC)c(OCC[C@@H]3C[C@@H]4CC[C@H]3C4)c2)C1=O] |
| 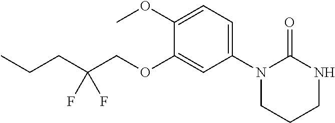 [SMILES: CCCC(F)(F)COc1cc(N2CCCNC2=O)ccc1OC] | 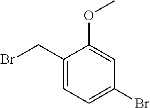 | 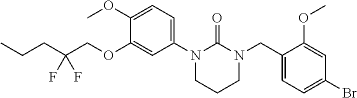 [SMILES: CCCC(F)(F)COc1cc(N2CCCN(Cc3ccc(Br)cc3OC)C2=O)ccc1OC] |
| 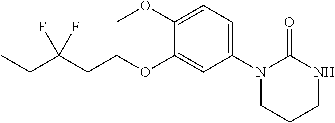 [SMILES: CCC(F)(F)CCOc1cc(N2CCCNC2=O)ccc1OC] | 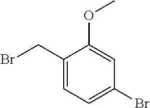 | 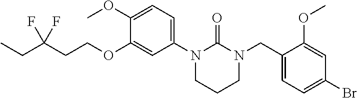 [SMILES: CCC(F)(F)CCOc1cc(N2CCCN(Cc3ccc(Br)cc3OC)C2=O)ccc1OC] |
| 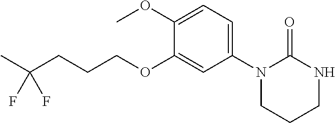 [SMILES: COc1ccc(N2CCCNC2=O)cc1OCCCC(C)(F)F] | 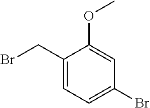 | 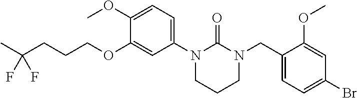 [SMILES: COc1cc(Br)ccc1CN1CCCN(c2ccc(OC)c(OCCCC(C)(F)F)c2)C1=O] |
| 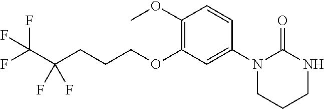 [SMILES: COc1ccc(N2CCCNC2=O)cc1OCCCC(F)(F)C(F)(F)F] | 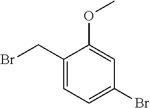 | 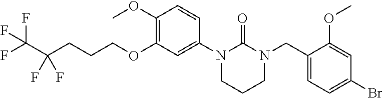 [SMILES: COc1cc(Br)ccc1CN1CCCN(c2ccc(OC)c(OCCCC(F)(F)C(F)(F)F)c2)C1=O] |
| 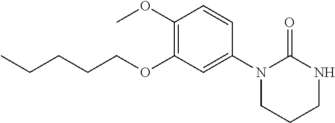 [SMILES: CCCCCOc1cc(N2CCCNC2=O)ccc1OC] | 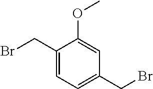 | 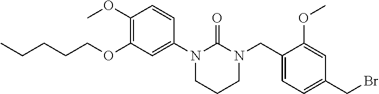 [SMILES: CCCCCOc1cc(N2CCCN(Cc3ccc(CBr)cc3OC)C2=O)ccc1OC] |
| 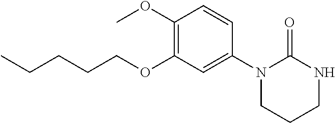 [SMILES: CCCCCOc1cc(N2CCCNC2=O)ccc1OC] | 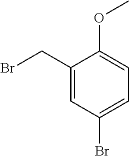 | 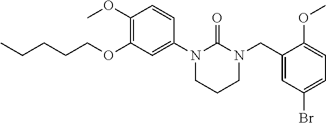 [SMILES: CCCCCOc1cc(N2CCCN(Cc3cc(Br)ccc3OC)C2=O)ccc1OC] |
| 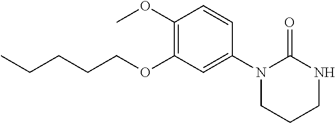 [SMILES: CCCCCOc1cc(N2CCCNC2=O)ccc1OC] | 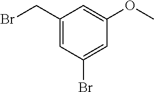 | 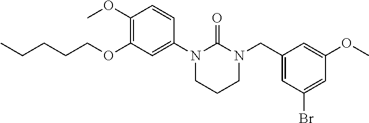 [SMILES: CCCCCOc1cc(N2CCCN(Cc3cc(Br)cc(OC)c3)C2=O)ccc1OC] |
| 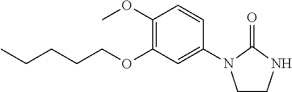 [SMILES: CCCCCOc1cc(N2CCNC2=O)ccc1OC] | 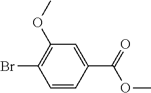 | 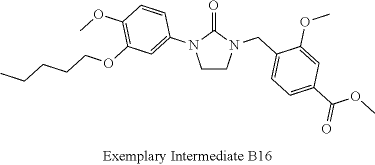 [SMILES: CCCCCOc1cc(N2CCN(Cc3ccc(C(=O)OC)cc3OC)C2=O)ccc1OC] |
| 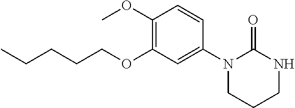 [SMILES: CCCCCOc1cc(N2CCCNC2=O)ccc1OC] | 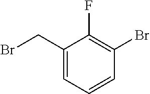 | 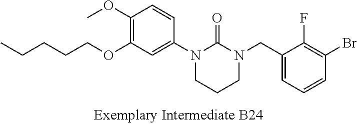 [SMILES: CCCCCOc1cc(N2CCCN(Cc3cccc(Br)c3F)C2=O)ccc1OC] |
| 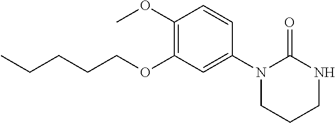 [SMILES: CCCCCOc1cc(N2CCCNC2=O)ccc1OC] | 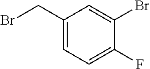 | 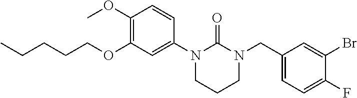 [SMILES: CCCCCOc1cc(N2CCCN(Cc3ccc(F)c(Br)c3)C2=O)ccc1OC] |
| 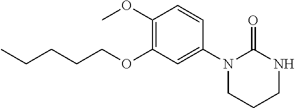 [SMILES: CCCCCOc1cc(N2CCCNC2=O)ccc1OC] | 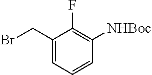 [SMILES: CC(C)(C)OC(=O)Nc1cccc(CBr)c1F] | 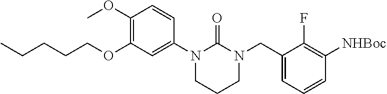 [SMILES: CCCCCOc1cc(N2CCCN(Cc3cccc(NC(=O)OC(C)(C)C)c3F)C2=O)ccc1OC] |
| 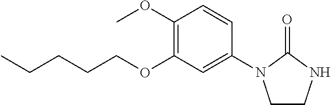 [SMILES: CCCCCOc1cc(N2CCNC2=O)ccc1OC] | 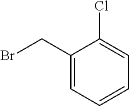 | 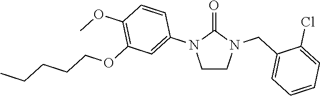 [SMILES: CCCCCOc1cc(N2CCN(Cc3ccccc3Cl)C2=O)ccc1OC] |
| 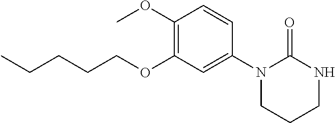 [SMILES: CCCCCOc1cc(N2CCCNC2=O)ccc1OC] | 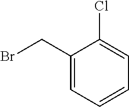 | 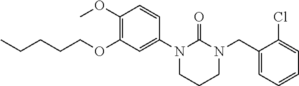 [SMILES: CCCCCOc1cc(N2CCCN(Cc3ccccc3Cl)C2=O)ccc1OC] |
| 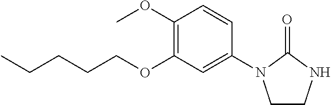 [SMILES: CCCCCOc1cc(N2CCNC2=O)ccc1OC] | 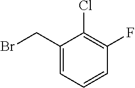 | 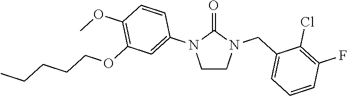 [SMILES: CCCCCOc1cc(N2CCN(Cc3cccc(F)c3Cl)C2=O)ccc1OC] |
| 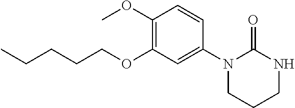 [SMILES: CCCCCOc1cc(N2CCCNC2=O)ccc1OC] | 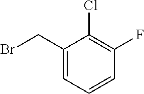 | 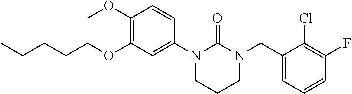 [SMILES: CCCCCOc1cc(N2CCCN(Cc3cccc(F)c3Cl)C2=O)ccc1OC] |
| 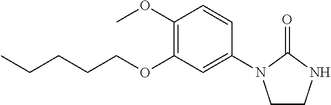 [SMILES: CCCCCOc1cc(N2CCNC2=O)ccc1OC] | 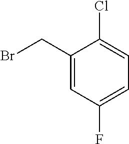 | 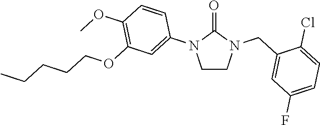 [SMILES: CCCCCOc1cc(N2CCN(Cc3cc(F)ccc3Cl)C2=O)ccc1OC] |
| 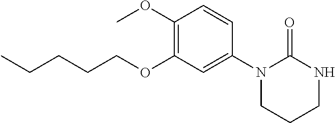 [SMILES: CCCCCOc1cc(N2CCCNC2=O)ccc1OC] | 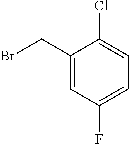 | 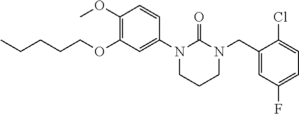 [SMILES: CCCCCOc1cc(N2CCCN(Cc3cc(F)ccc3Cl)C2=O)ccc1OC] |
| 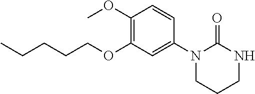 [SMILES: CCCCCOc1cc(N2CCCNC2=O)ccc1OC] | 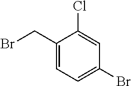 | 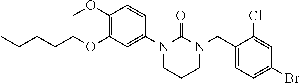 [SMILES: CCCCCOc1cc(N2CCCN(Cc3ccc(Br)cc3Cl)C2=O)ccc1OC] |
| 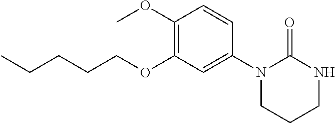 [SMILES: CCCCCOc1cc(N2CCCNC2=O)ccc1OC] | 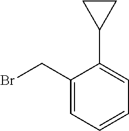 [SMILES: BrCc1ccccc1C1CC1] | 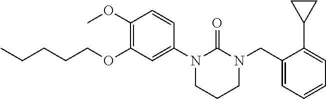 [SMILES: CCCCCOc1cc(N2CCCN(Cc3ccccc3C3CC3)C2=O)ccc1OC] |
| 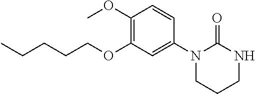 [SMILES: CCCCCOc1cc(N2CCCNC2=O)ccc1OC] | 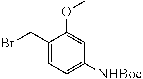 [SMILES: COc1cc(NC(=O)OC(C)(C)C)ccc1CBr] | 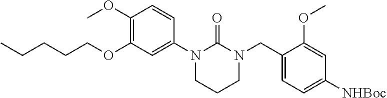 [SMILES: CCCCCOc1cc(N2CCCN(Cc3ccc(NC(=O)OC(C)(C)C)cc3OC)C2=O)ccc1OC] |
| 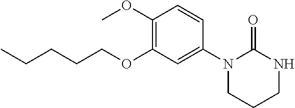 [SMILES: CCCCCOc1cc(N2CCCNC2=O)ccc1OC] | 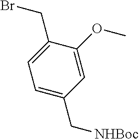 [SMILES: COc1cc(CNC(=O)OC(C)(C)C)ccc1CBr] | 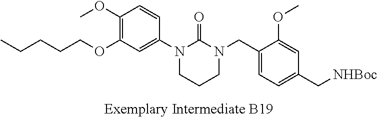 [SMILES: CCCCCOc1cc(N2CCCN(Cc3ccc(CNC(=O)OC(C)(C)C)cc3OC)C2=O)ccc1OC] |
| 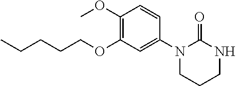 [SMILES: CCCCCOc1cc(N2CCCNC2=O)ccc1OC] | 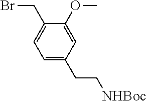 [SMILES: COc1cc(CCNC(=O)OC(C)(C)C)ccc1CBr] | 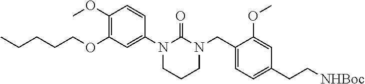 [SMILES: CCCCCOc1cc(N2CCCN(Cc3ccc(CCNC(=O)OC(C)(C)C)cc3OC)C2=O)ccc1OC] |
| 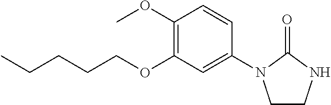 [SMILES: CCCCCOc1cc(N2CCNC2=O)ccc1OC] | 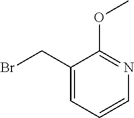 | 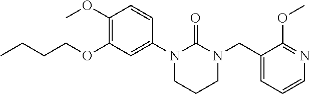 [SMILES: CCCCOc1cc(N2CCCN(Cc3cccnc3OC)C2=O)ccc1OC] |
| 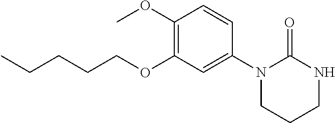 [SMILES: CCCCCOc1cc(N2CCCNC2=O)ccc1OC] | 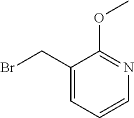 | 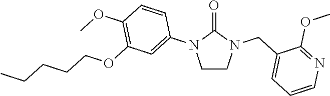 [SMILES: CCCCCOc1cc(N2CCN(Cc3cccnc3OC)C2=O)ccc1OC] |
| 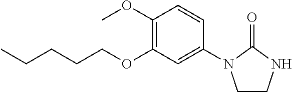 [SMILES: CCCCCOc1cc(N2CCNC2=O)ccc1OC] | 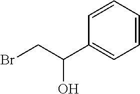 | 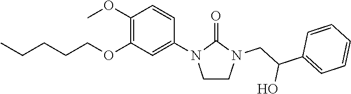 [SMILES: CCCCCOc1cc(N2CCN(CC(O)c3ccccc3)C2=O)ccc1OC] |
| 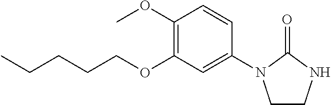 [SMILES: CCCCCOc1cc(N2CCNC2=O)ccc1OC] | 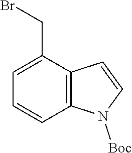 [SMILES: CC(C)(C)OC(=O)n1ccc2c(CBr)cccc21] | 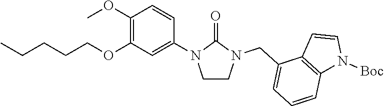 [SMILES: CCCCCOc1cc(N2CCN(Cc3cccc4c3ccn4C(=O)OC(C)(C)C)C2=O)ccc1OC] |
| 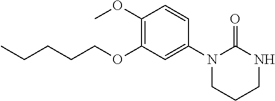 [SMILES: CCCCCOc1cc(N2CCCNC2=O)ccc1OC] | 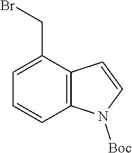 [SMILES: CC(C)(C)OC(=O)n1ccc2c(CBr)cccc21] | 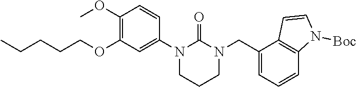 [SMILES: CCCCCOc1cc(N2CCCN(Cc3cccc4c3ccn4C(=O)OC(C)(C)C)C2=O)ccc1OC] |
| 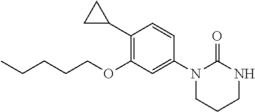 [SMILES: CCCCCOc1cc(N2CCCNC2=O)ccc1C1CC1] | 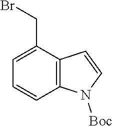 [SMILES: CC(C)(C)OC(=O)n1ccc2c(CBr)cccc21] | 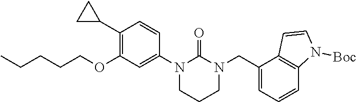 [SMILES: CCCCCOc1cc(N2CCCN(Cc3cccc4c3ccn4C(=O)OC(C)(C)C)C2=O)ccc1C1CC1] |
| 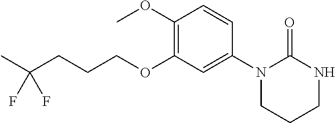 [SMILES: COc1ccc(N2CCCNC2=O)cc1OCCCC(C)(F)F] | 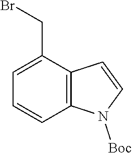 [SMILES: CC(C)(C)OC(=O)n1ccc2c(CBr)cccc21] | 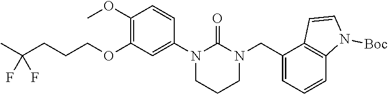 [SMILES: COc1ccc(N2CCCN(Cc3cccc4c3ccn4C(=O)OC(C)(C)C)C2=O)cc1OCCCC(C)(F)F] |
| 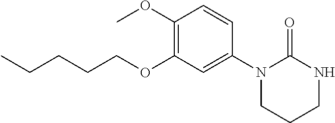 [SMILES: CCCCCOc1cc(N2CCCNC2=O)ccc1OC] | 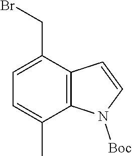 [SMILES: Cc1ccc(CBr)c2ccn(C(=O)OC(C)(C)C)c12] | 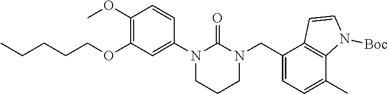 [SMILES: CCCCCOc1cc(N2CCCN(Cc3ccc(C)c4c3ccn4C(=O)OC(C)(C)C)C2=O)ccc1OC] |
| 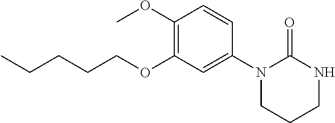 [SMILES: CCCCCOc1cc(N2CCCNC2=O)ccc1OC] | 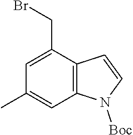 [SMILES: Cc1cc(CBr)c2ccn(C(=O)OC(C)(C)C)c2c1] | 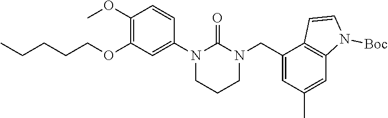 [SMILES: CCCCCOc1cc(N2CCCN(Cc3cc(C)cc4c3ccn4C(=O)OC(C)(C)C)C2=O)ccc1OC] |
| 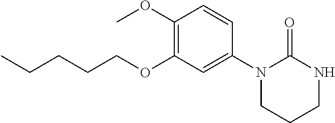 [SMILES: CCCCCOc1cc(N2CCCNC2=O)ccc1OC] | 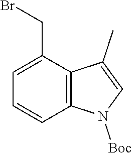 [SMILES: Cc1cn(C(=O)OC(C)(C)C)c2cccc(CBr)c12] | 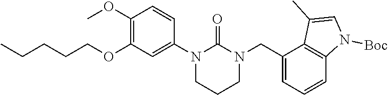 [SMILES: CCCCCOc1cc(N2CCCN(Cc3cccc4c3c(C)cn4C(=O)OC(C)(C)C)C2=O)ccc1OC] |
| 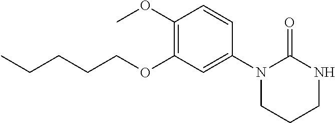 [SMILES: CCCCCOc1cc(N2CCCNC2=O)ccc1OC] | 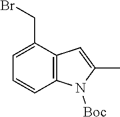 [SMILES: Cc1cc2c(CBr)cccc2n1C(=O)OC(C)(C)C] | 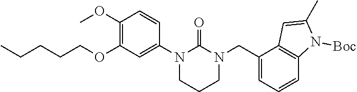 [SMILES: CCCCCOc1cc(N2CCCN(Cc3cccc4c3cc(C)n4C(=O)OC(C)(C)C)C2=O)ccc1OC] |
| 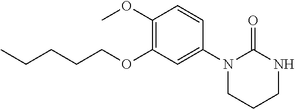 [SMILES: CCCCCOc1cc(N2CCCNC2=O)ccc1OC] | 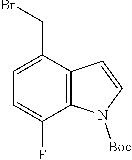 [SMILES: CC(C)(C)OC(=O)n1ccc2c(CBr)ccc(F)c21] | 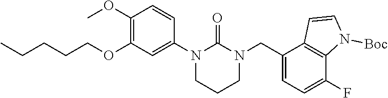 [SMILES: CCCCCOc1cc(N2CCCN(Cc3ccc(F)c4c3ccn4C(=O)OC(C)(C)C)C2=O)ccc1OC] |
| 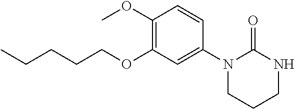 [SMILES: CCCCCOc1cc(N2CCCNC2=O)ccc1OC] | 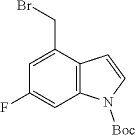 [SMILES: CC(C)(C)OC(=O)n1ccc2c(CBr)cc(F)cc21] | 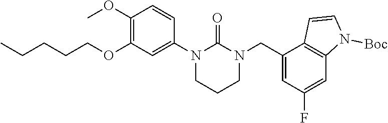 [SMILES: CCCCCOc1cc(N2CCCN(Cc3cc(F)cc4c3ccn4C(=O)OC(C)(C)C)C2=O)ccc1OC] |
| 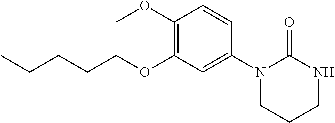 [SMILES: CCCCCOc1cc(N2CCCNC2=O)ccc1OC] | 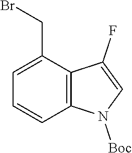 [SMILES: CC(C)(C)OC(=O)n1cc(F)c2c(CBr)cccc21] | 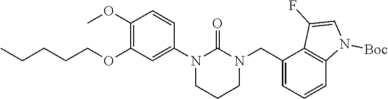 [SMILES: CCCCCOc1cc(N2CCCN(Cc3cccc4c3c(F)cn4C(=O)OC(C)(C)C)C2=O)ccc1OC] |
| 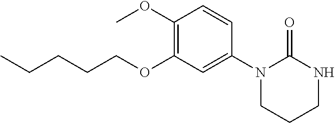 [SMILES: CCCCCOc1cc(N2CCCNC2=O)ccc1OC] | 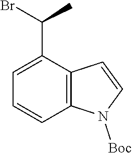 [SMILES: C[C@H](Br)c1cccc2c1ccn2C(=O)OC(C)(C)C] | 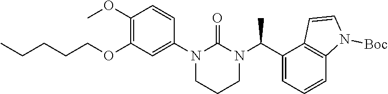 [SMILES: CCCCCOc1cc(N2CCCN([C@@H](C)c3cccc4c3ccn4C(=O)OC(C)(C)C)C2=O)ccc1OC] |
| 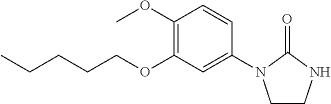 [SMILES: CCCCCOc1cc(N2CCNC2=O)ccc1OC] | 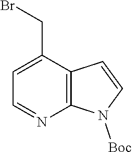 [SMILES: CC(C)(C)OC(=O)n1ccc2c(CBr)ccnc21] | 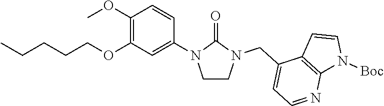 [SMILES: CCCCCOc1cc(N2CCN(Cc3ccnc4c3ccn4C(=O)OC(C)(C)C)C2=O)ccc1OC] |
| 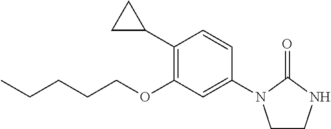 [SMILES: CCCCCOc1cc(N2CCNC2=O)ccc1C1CC1] | 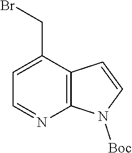 [SMILES: CC(C)(C)OC(=O)n1ccc2c(CBr)ccnc21] | 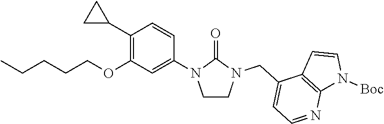 [SMILES: CCCCCOc1cc(N2CCN(Cc3ccnc4c3ccn4C(=O)OC(C)(C)C)C2=O)ccc1C1CC1] |
| 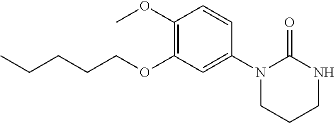 [SMILES: CCCCCOc1cc(N2CCCNC2=O)ccc1OC] | 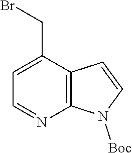 [SMILES: CC(C)(C)OC(=O)n1ccc2c(CBr)ccnc21] | 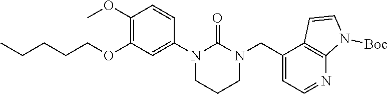 [SMILES: CCCCCOc1cc(N2CCCN(Cc3ccnc4c3ccn4C(=O)OC(C)(C)C)C2=O)ccc1OC] |
| 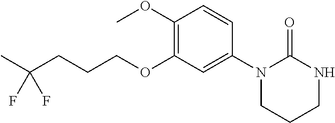 [SMILES: COc1ccc(N2CCCNC2=O)cc1OCCCC(C)(F)F] | 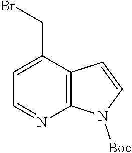 [SMILES: CC(C)(C)OC(=O)n1ccc2c(CBr)ccnc21] | 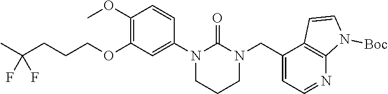 [SMILES: COc1ccc(N2CCCN(Cc3ccnc4c3ccn4C(=O)OC(C)(C)C)C2=O)cc1OCCCC(C)(F)F] |
| 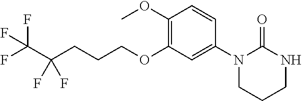 [SMILES: COc1ccc(N2CCCNC2=O)cc1OCCCC(F)(F)C(F)(F)F] | 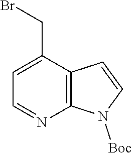 [SMILES: CC(C)(C)OC(=O)n1ccc2c(CBr)ccnc21] | 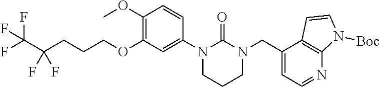 [SMILES: COc1ccc(N2CCCN(Cc3ccnc4c3ccn4C(=O)OC(C)(C)C)C2=O)cc1OCCCC(F)(F)C(F)(F)F] |
| 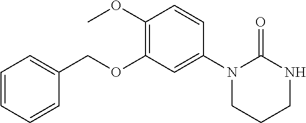 [SMILES: COc1ccc(N2CCCNC2=O)cc1OCc1ccccc1] | 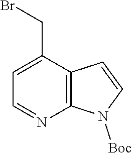 [SMILES: CC(C)(C)OC(=O)n1ccc2c(CBr)ccnc21] | 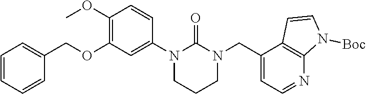 [SMILES: COc1ccc(N2CCCN(Cc3ccnc4c3ccn4C(=O)OC(C)(C)C)C2=O)cc1OCc1ccccc1] |
| 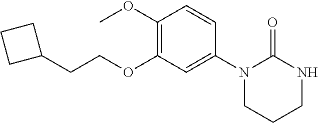 [SMILES: COc1ccc(N2CCCNC2=O)cc1OCCC1CCC1] | 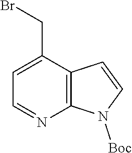 [SMILES: CC(C)(C)OC(=O)n1ccc2c(CBr)ccnc21] | 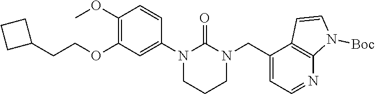 [SMILES: COc1ccc(N2CCCN(Cc3ccnc4c3ccn4C(=O)OC(C)(C)C)C2=O)cc1OCCC1CCC1] |
| 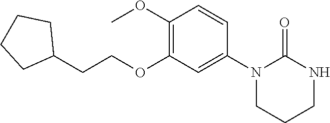 [SMILES: COc1ccc(N2CCCNC2=O)cc1OCCC1CCCC1] | 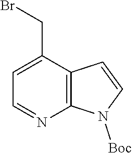 [SMILES: CC(C)(C)OC(=O)n1ccc2c(CBr)ccnc21] | 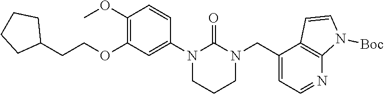 [SMILES: COc1ccc(N2CCCN(Cc3ccnc4c3ccn4C(=O)OC(C)(C)C)C2=O)cc1OCCC1CCCC1] |
| 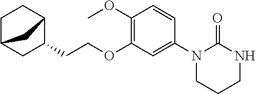 [SMILES: COc1ccc(N2CCCNC2=O)cc1OCC[C@@H]1C[C@@H]2CC[C@H]1C2] | 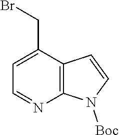 [SMILES: CC(C)(C)OC(=O)n1ccc2c(CBr)ccnc21] | 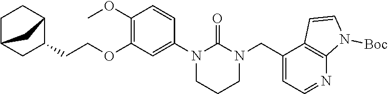 [SMILES: COc1ccc(N2CCCN(Cc3ccnc4c3ccn4C(=O)OC(C)(C)C)C2=O)cc1OCC[C@@H]1C[C@@H]2CC[C@H]1C2] |
| 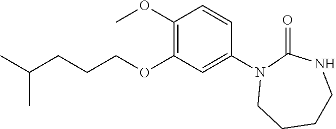 [SMILES: COc1ccc(N2CCCCNC2=O)cc1OCCCC(C)C] | 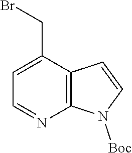 [SMILES: CC(C)(C)OC(=O)n1ccc2c(CBr)ccnc21] | 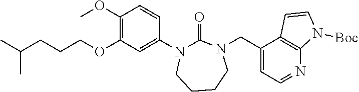 [SMILES: COc1ccc(N2CCCCN(Cc3ccnc4c3ccn4C(=O)OC(C)(C)C)C2=O)cc1OCCCC(C)C] |
| 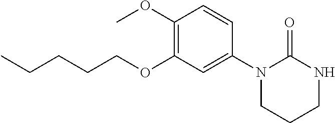 [SMILES: CCCCCOc1cc(N2CCCNC2=O)ccc1OC] | 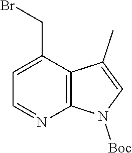 [SMILES: Cc1cn(C(=O)OC(C)(C)C)c2nccc(CBr)c12] | 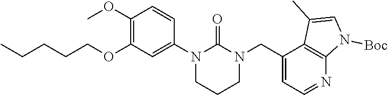 [SMILES: CCCCCOc1cc(N2CCCN(Cc3ccnc4c3c(C)cn4C(=O)OC(C)(C)C)C2=O)ccc1OC] |
| 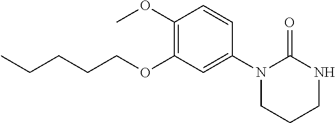 [SMILES: CCCCCOc1cc(N2CCCNC2=O)ccc1OC] | 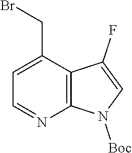 [SMILES: CC(C)(C)OC(=O)n1cc(F)c2c(CBr)ccnc21] | 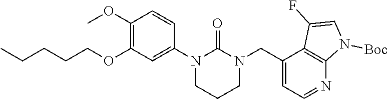 [SMILES: CCCCCOc1cc(N2CCCN(Cc3ccnc4c3c(F)cn4C(=O)OC(C)(C)C)C2=O)ccc1OC] |
| 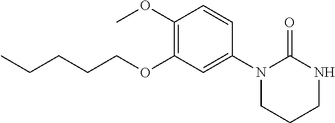 [SMILES: CCCCCOc1cc(N2CCCNC2=O)ccc1OC] | 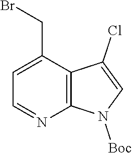 [SMILES: CC(C)(C)OC(=O)n1cc(Cl)c2c(CBr)ccnc21] | 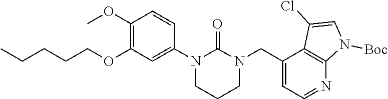 [SMILES: CCCCCOc1cc(N2CCCN(Cc3ccnc4c3c(Cl)cn4C(=O)OC(C)(C)C)C2=O)ccc1OC] |
| 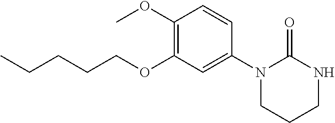 [SMILES: CCCCCOc1cc(N2CCCNC2=O)ccc1OC] | 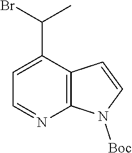 [SMILES: CC(Br)c1ccnc2c1ccn2C(=O)OC(C)(C)C] | 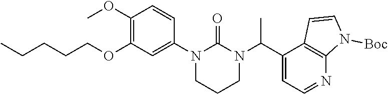 [SMILES: CCCCCOc1cc(N2CCCN(C(C)c3ccnc4c3ccn4C(=O)OC(C)(C)C)C2=O)ccc1OC] |
| 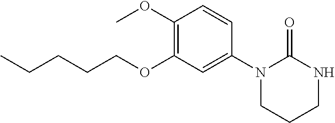 [SMILES: CCCCCOc1cc(N2CCCNC2=O)ccc1OC] | 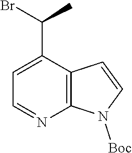 [SMILES: C[C@H](Br)c1ccnc2c1ccn2C(=O)OC(C)(C)C] | 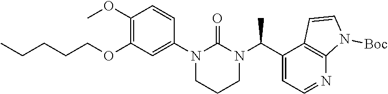 [SMILES: CCCCCOc1cc(N2CCCN([C@@H](C)c3ccnc4c3ccn4C(=O)OC(C)(C)C)C2=O)ccc1OC] |
| 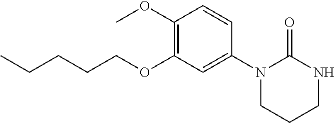 [SMILES: CCCCCOc1cc(N2CCCNC2=O)ccc1OC] | 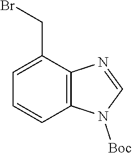 [SMILES: CC(C)(C)OC(=O)n1cnc2c(CBr)cccc21] | 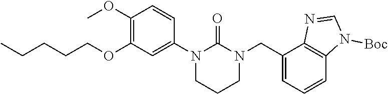 [SMILES: CCCCCOc1cc(N2CCCN(Cc3cccc4c3ncn4C(=O)OC(C)(C)C)C2=O)ccc1OC] |
| 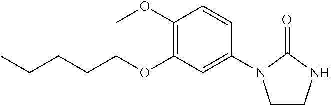 [SMILES: CCCCCOc1cc(N2CCNC2=O)ccc1OC] | 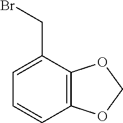 [SMILES: BrCc1cccc2c1OCO2] | 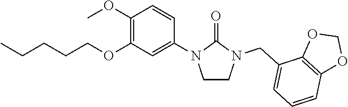 [SMILES: CCCCCOc1cc(N2CCN(Cc3cccc4c3OCO4)C2=O)ccc1OC] |
| 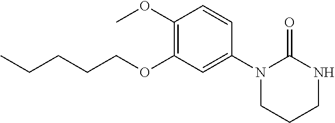 [SMILES: CCCCCOc1cc(N2CCCNC2=O)ccc1OC] | 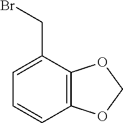 [SMILES: BrCc1cccc2c1OCO2] | 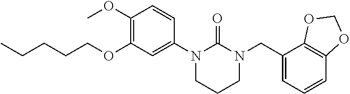 [SMILES: CCCCCOc1cc(N2CCCN(Cc3cccc4c3OCO4)C2=O)ccc1OC] |
| 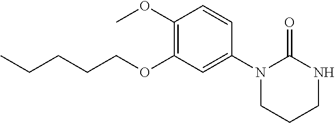 [SMILES: CCCCCOc1cc(N2CCCNC2=O)ccc1OC] | 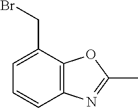 | 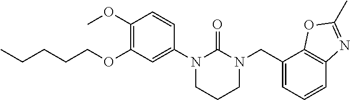 [SMILES: CCCCCOc1cc(N2CCCN(Cc3cccc4nc(C)oc34)C2=O)ccc1OC] |
| 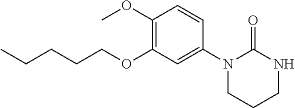 [SMILES: CCCCCOc1cc(N2CCCNC2=O)ccc1OC] | 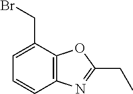 | 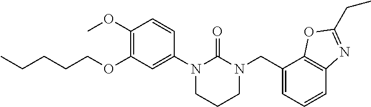 [SMILES: CCCCCOc1cc(N2CCCN(Cc3cccc4nc(CC)oc34)C2=O)ccc1OC] |
| 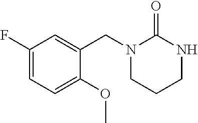 [SMILES: COc1ccc(F)cc1CN1CCCNC1=O] | 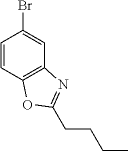 | 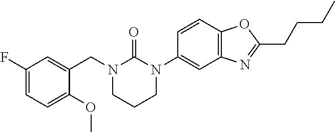 [SMILES: CCCCc1nc2cc(N3CCCN(Cc4cc(F)ccc4OC)C3=O)ccc2o1] |

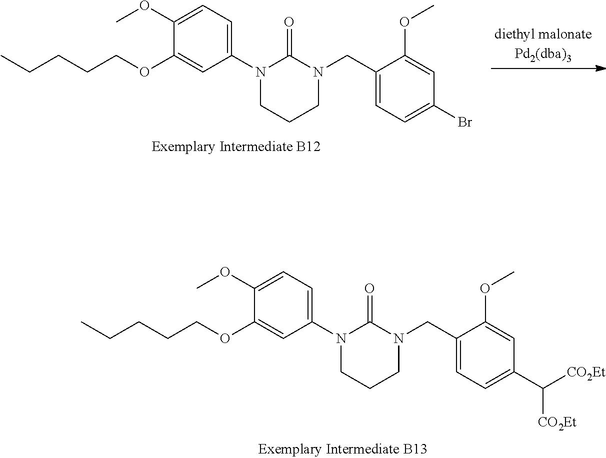 [SMILES: CCCCCOc1cc(N2CCCN(Cc3ccc(Br)cc3OC)C2=O)ccc1OC ; CCCCCOc1cc(N2CCCN(Cc3ccc(C(C(=O)OCC)C(=O)OCC)cc3OC)C2=O)ccc1OC]

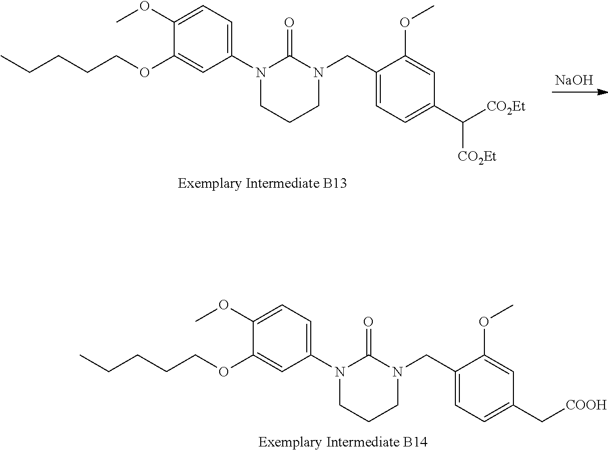 [SMILES: CCCCCOc1cc(N2CCCN(Cc3ccc(C(C(=O)OCC)C(=O)OCC)cc3OC)C2=O)ccc1OC ; "NaOH" ; CCCCCOc1cc(N2CCCN(Cc3ccc(CC(=O)O)cc3OC)C2=O)ccc1OC]

Cesium carbonate (16.9 g, 68 mmol) is added to a solution of Exemplary Intermediate B12 1-(4-bromo-2-methoxybenzyl)-3-(4-methoxy-3-(pentyloxy)phenyl) tetrahydropyrimidin-2(1H)-one) (17.0 g, 34 mmol) and diethyl malonate (10.9 g, 68 mmol) in anhydrous N,N-dimethylformamide (150 mL). The mixture is sparged with dry nitrogen for 5 min, then tris(dibenzylideneacetone)dipalladium(0) (1.0 g, 1.1 mmol) and SPhos ligand (1.0 g, 2.44 mmol) are added. The mixture is heated to 95° C. and is stirred at this temperature for 12 h. After cooling to room temperature, the mixture is quenched with water (500 mL) and extracted with ethyl acetate (3×300 mL). The combined organic phases are washed with brine (3×300 mL), dried over sodium sulfate, filtered, and concentrated in vacuo. The residue is purified by column chromatography over silica gel (hexanes/ethyl acetate: 5:1 to 3:1) to afford Exemplary Intermediate B13 (diethyl 2-(3-methoxy-4-((3-(4-methoxy-3-(pentyloxy)phenyl)-2-oxotetrahydropyrimidin-1(2H)-yl)methyl)phenyl)malonate; 14.0 g, 70% yield).

A solution of Exemplary Intermediate B13 (diethyl 2-(3-methoxy-4-((3-(4-methoxy-3-(pentyloxy)phenyl)-2-oxotetrahydropyrimidin-1(2H)-yl)methyl)phenyl)malonate; 14.0 g, 24 mmol) and sodium hydroxide (2.0 g, 50 mmol) in a mixture of ethanol and water (1:2 v/v, 300 mL) is refluxed for 12 h. After cooling to room temperature, the mixture is washed with ethyl acetate/hexane mixture (1:1 v/v, 3×200 mL) and these washes are discarded. The remaining aqueous layer is acidified to pH of 3 with 1N hydrochloric acid then refluxed for 2 h. After cooling to room temperature, the mixture is extracted with ethyl acetate (3×300 mL). The combined organic phases are washed with brine (3×200 mL), then dried over sodium sulfate, filtered, and concentrated to afford Exemplary Intermediate B14 (2-(3-methoxy-4-((3-(4-methoxy-3-(pentyloxy)phenyl)-2-oxotetrahydropyrimidin-1(2H)-yl)methyl)phenyl)acetic acid; 9.2 g, 81% yield).

The following compounds are prepared in a similar manner as Exemplary Intermediate B14, as described above.

Bromide

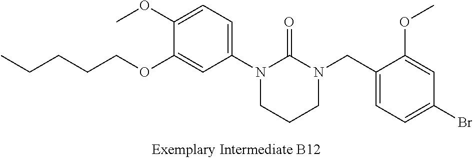 [SMILES: CCCCCOc1cc(N2CCCN(Cc3ccc(Br)cc3OC)C2=O)ccc1OC]

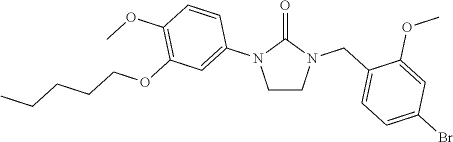 [SMILES: CCCCCOc1cc(N2CCN(Cc3ccc(Br)cc3OC)C2=O)ccc1OC]

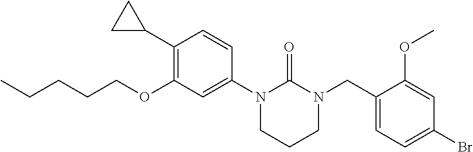 [SMILES: CCCCCOc1cc(N2CCCN(Cc3ccc(Br)cc3OC)C2=O)ccc1C1CC1]

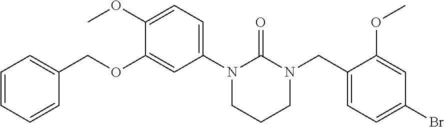 [SMILES: COc1cc(Br)ccc1CN1CCCN(c2ccc(OC)c(OCc3ccccc3)c2)C1=O]

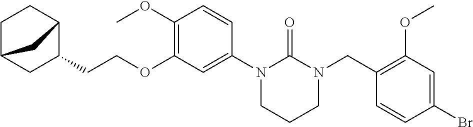 [SMILES: COc1cc(Br)ccc1CN1CCCN(c2ccc(OC)c(OCC[C@@H]3C[C@@H]4CC[C@H]3C4)c2)C1=O]

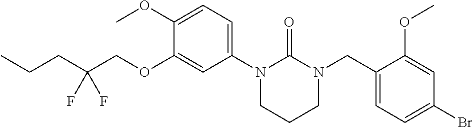 [SMILES: CCCC(F)(F)COc1cc(N2CCCN(Cc3ccc(Br)cc3OC)C2=O)ccc1OC]

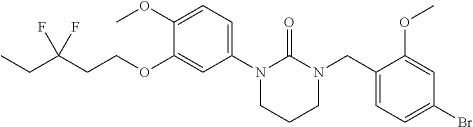 [SMILES: CCC(F)(F)CCOc1cc(N2CCCN(Cc3ccc(Br)cc3OC)C2=O)ccc1OC]

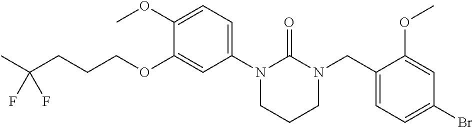 [SMILES: COc1cc(Br)ccc1CN1CCCN(c2ccc(OC)c(OCCCC(C)(F)F)c2)C1=O]

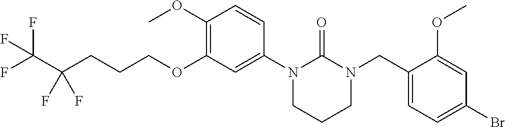 [SMILES: COc1cc(Br)ccc1CN1CCCN(c2ccc(OC)c(OCCCC(F)(F)C(F)(F)F)c2)C1=O]

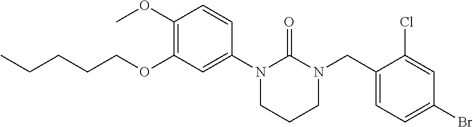 [SMILES: CCCCCOc1cc(N2CCCN(Cc3ccc(Br)cc3Cl)C2=O)ccc1OC]

Compound

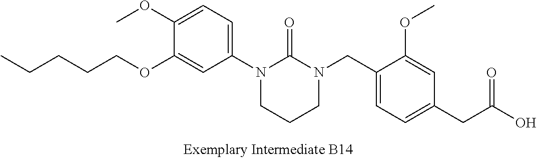 [SMILES: CCCCCOc1cc(N2CCCN(Cc3ccc(CC(=O)O)cc3OC)C2=O)ccc1OC]

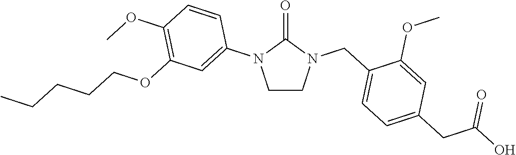 [SMILES: CCCCCOc1cc(N2CCN(Cc3ccc(CC(=O)O)cc3OC)C2=O)ccc1OC]

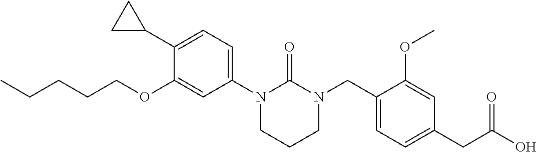 [SMILES: CCCCCOc1cc(N2CCCN(Cc3ccc(CC(=O)O)cc3OC)C2=O)ccc1C1CC1]

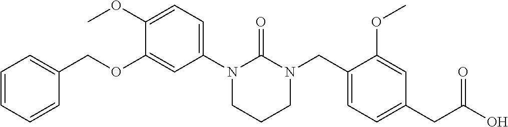 [SMILES: COc1cc(CC(=O)O)ccc1CN1CCCN(c2ccc(OC)c(OCc3ccccc3)c2)C1=O]

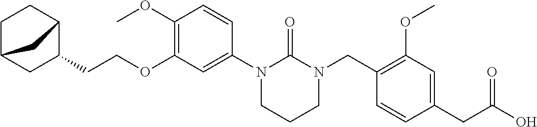 [SMILES: COc1cc(CC(=O)O)ccc1CN1CCCN(c2ccc(OC)c(OCC[C@@H]3C[C@@H]4CC[C@H]3C4)c2)C1=O]

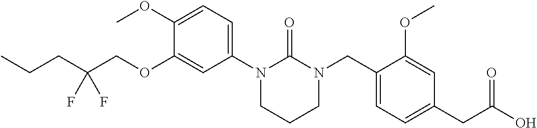 [SMILES: CCCC(F)(F)COc1cc(N2CCCN(Cc3ccc(CC(=O)O)cc3OC)C2=O)ccc1OC]

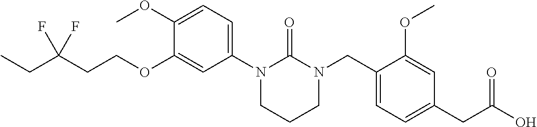 [SMILES: CCC(F)(F)CCOc1cc(N2CCCN(Cc3ccc(CC(=O)O)cc3OC)C2=O)ccc1OC]

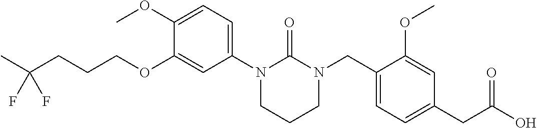 [SMILES: COc1cc(CC(=O)O)ccc1CN1CCCN(c2ccc(OC)c(OCCCC(C)(F)F)c2)C1=O]

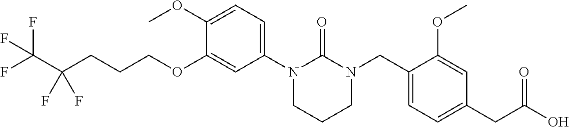 [SMILES: COc1cc(CC(=O)O)ccc1CN1CCCN(c2ccc(OC)c(OCCCC(F)(F)C(F)(F)F)c2)C1=O]

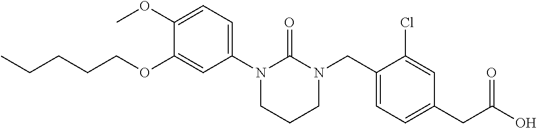 [SMILES: CCCCCOc1cc(N2CCCN(Cc3ccc(CC(=O)O)cc3Cl)C2=O)ccc1OC]

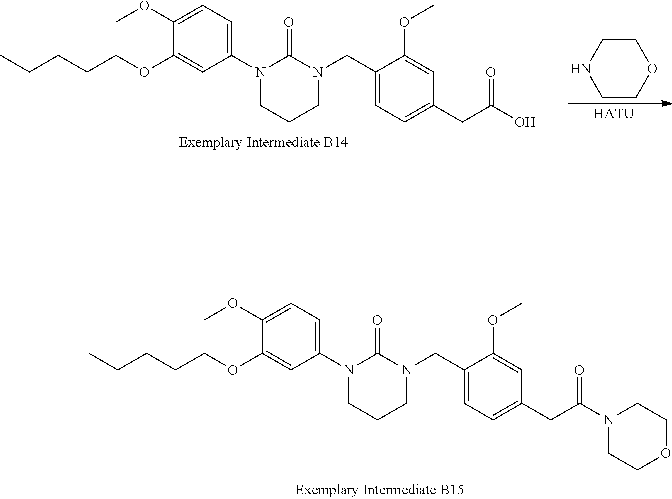

Exemplary Intermediate B14 (2-(3-methoxy-4-((3-(4-methoxy-3-(pentyloxy)phenyl)-2-oxotetrahydropyrimidin-1(2H)-yl)methyl)phenyl)acetic acid) is dissolved in N,N-dimethylformamide, then HATU, 1,4-morpholine, and N,N-diisopropylethylamine were added. Reaction mixture is stirred at room temperature for 2 h. Water is added to reaction mixture, and it is extracted three times with ethyl acetate. The organic layers are dried over a phase separator and concentrated in vacuo. The residue is purified by column chromatography over silica (dichloromethane/methanol: 100/0 to 97/3) to afford Exemplary Intermediate B15 (1-(4-methoxy-3-(pentyloxy)phenyl)-3-(2-methoxy-4-(2-morpholino-2-oxoethyl)benzyl) tetrahydropyrimidin-2(1H)-one).

The following compounds are prepared in a similar manner as Exemplary Intermediate B15, as described above.

| Acid | Amine |
|---|---|
| 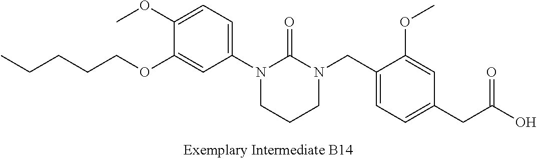 [SMILES: CCCCCOc1cc(N2CCCN(Cc3ccc(CC(=O)O)cc3OC)C2=O)ccc1OC] | 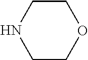 |
| 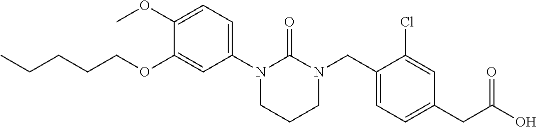 [SMILES: CCCCCOc1cc(N2CCCN(Cc3ccc(CC(=O)O)cc3Cl)C2=O)ccc1OC] | 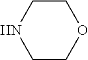 |
| 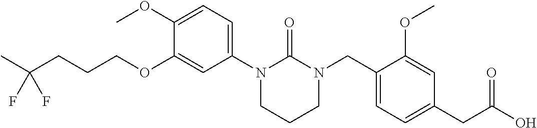 [SMILES: COc1cc(CC(=O)O)ccc1CN1CCCN(c2ccc(OC)c(OCCCC(C)(F)F)c2)C1=O] | 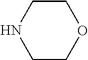 |
| 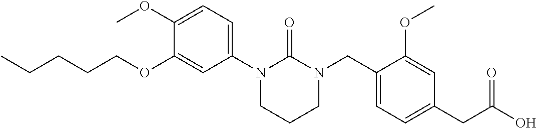 [SMILES: CCCCCOc1cc(N2CCCN(Cc3ccc(CC(=O)O)cc3OC)C2=O)ccc1OC] | NH3 |
| 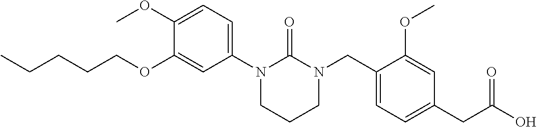 [SMILES: CCCCCOc1cc(N2CCCN(Cc3ccc(CC(=O)O)cc3OC)C2=O)ccc1OC] | 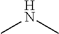 |
| 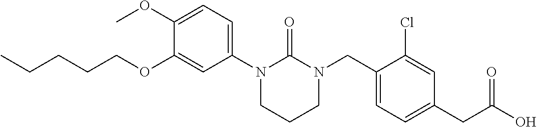 [SMILES: CCCCCOc1cc(N2CCCN(Cc3ccc(CC(=O)O)cc3Cl)C2=O)ccc1OC] | 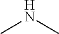 |
| 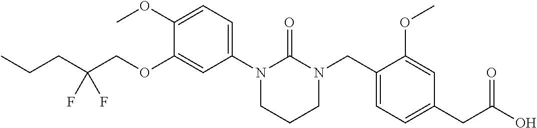 [SMILES: CCCC(F)(F)COc1cc(N2CCCN(Cc3ccc(CC(=O)O)cc3OC)C2=O)ccc1OC] | 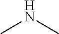 |
| 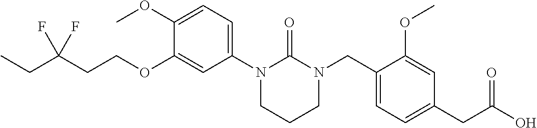 [SMILES: CCC(F)(F)CCOc1cc(N2CCCN(Cc3ccc(CC(=O)O)cc3OC)C2=O)ccc1OC] | 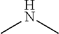 |
| 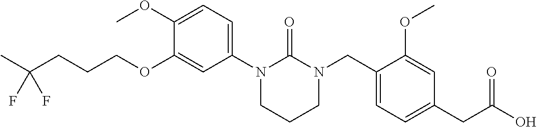 [SMILES: COc1cc(CC(=O)O)ccc1CN1CCCN(c2ccc(OC)c(OCCCC(C)(F)F)c2)C1=O] | 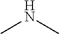 |
| 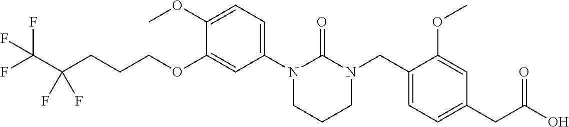 [SMILES: COc1cc(CC(=O)O)ccc1CN1CCCN(c2ccc(OC)c(OCCCC(F)(F)C(F)(F)F)c2)C1=O] | 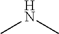 |
| 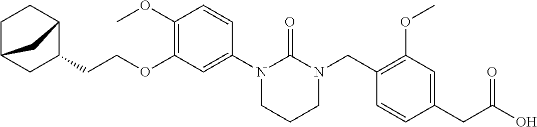 [SMILES: COc1cc(CC(=O)O)ccc1CN1CCCN(c2ccc(OC)c(OCC[C@@H]3C[C@@H]4CC[C@H]3C4)c2)C1=O] | 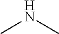 |
| 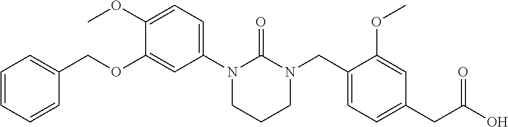 [SMILES: COc1cc(CC(=O)O)ccc1CN1CCCN(c2ccc(OC)c(OCc3ccccc3)c2)C1=O] | 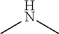 |
| 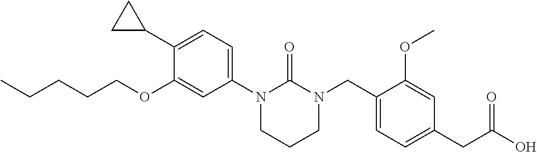 [SMILES: CCCCCOc1cc(N2CCCN(Cc3ccc(CC(=O)O)cc3OC)C2=O)ccc1C1CC1] | 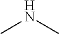 |
| 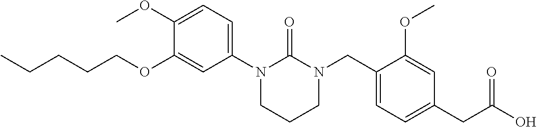 [SMILES: CCCCCOc1cc(N2CCCN(Cc3ccc(CC(=O)O)cc3OC)C2=O)ccc1OC] | 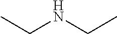 |
| 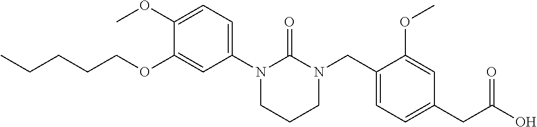 [SMILES: CCCCCOc1cc(N2CCCN(Cc3ccc(CC(=O)O)cc3OC)C2=O)ccc1OC] | 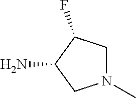 |
| 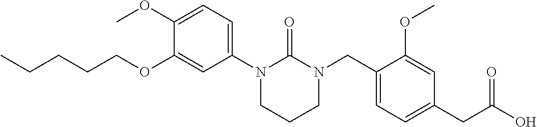 [SMILES: CCCCCOc1cc(N2CCCN(Cc3ccc(CC(=O)O)cc3OC)C2=O)ccc1OC] | 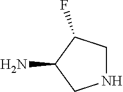 |
| 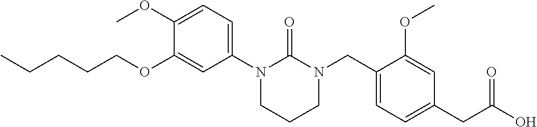 [SMILES: CCCCCOc1cc(N2CCCN(Cc3ccc(CC(=O)O)cc3OC)C2=O)ccc1OC] | 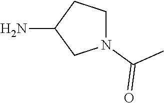 |
| 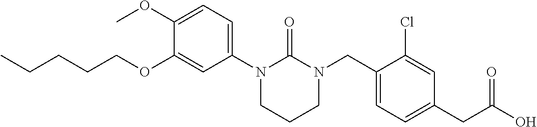 [SMILES: CCCCCOc1cc(N2CCCN(Cc3ccc(CC(=O)O)cc3Cl)C2=O)ccc1OC] | 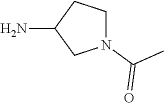 |
| 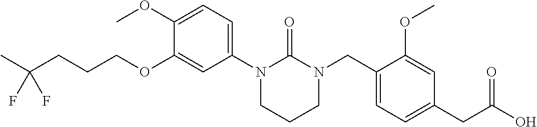 [SMILES: COc1cc(CC(=O)O)ccc1CN1CCCN(c2ccc(OC)c(OCCCC(C)(F)F)c2)C1=O] | 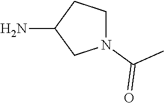 |
| 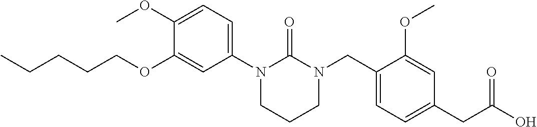 [SMILES: CCCCCOc1cc(N2CCCN(Cc3ccc(CC(=O)O)cc3OC)C2=O)ccc1OC] | 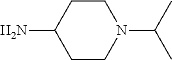 |
| 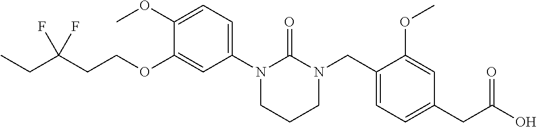 [SMILES: CCC(F)(F)CCOc1cc(N2CCCN(Cc3ccc(CC(=O)O)cc3OC)C2=O)ccc1OC] | 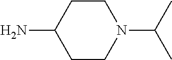 |
| 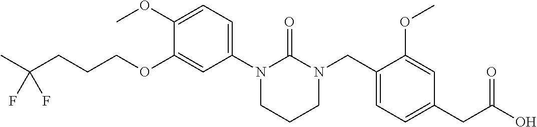 [SMILES: COc1cc(CC(=O)O)ccc1CN1CCCN(c2ccc(OC)c(OCCCC(C)(F)F)c2)C1=O] | 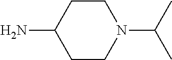 |
| 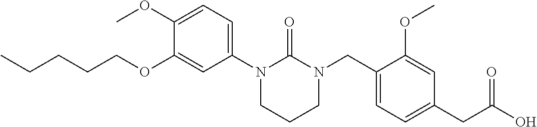 [SMILES: CCCCCOc1cc(N2CCCN(Cc3ccc(CC(=O)O)cc3OC)C2=O)ccc1OC] | 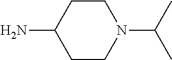 |
| 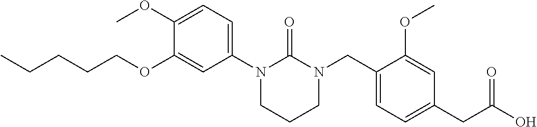 [SMILES: CCCCCOc1cc(N2CCCN(Cc3ccc(CC(=O)O)cc3OC)C2=O)ccc1OC] | 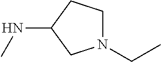 |
| 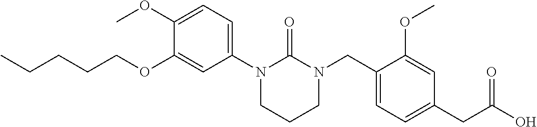 [SMILES: CCCCCOc1cc(N2CCCN(Cc3ccc(CC(=O)O)cc3OC)C2=O)ccc1OC] | 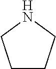 |
| 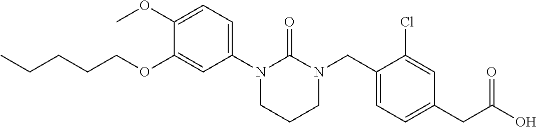 [SMILES: CCCCCOc1cc(N2CCCN(Cc3ccc(CC(=O)O)cc3Cl)C2=O)ccc1OC] | 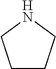 |
| 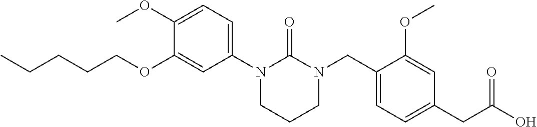 [SMILES: CCCCCOc1cc(N2CCCN(Cc3ccc(CC(=O)O)cc3OC)C2=O)ccc1OC] | 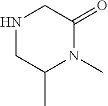 |
| 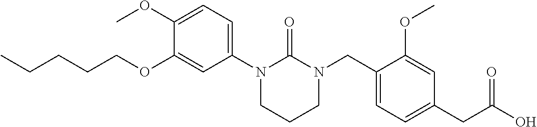 [SMILES: CCCCCOc1cc(N2CCCN(Cc3ccc(CC(=O)O)cc3OC)C2=O)ccc1OC] | 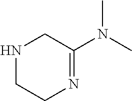 |
| 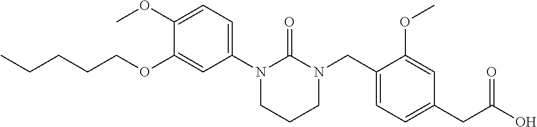 [SMILES: CCCCCOc1cc(N2CCCN(Cc3ccc(CC(=O)O)cc3OC)C2=O)ccc1OC] | 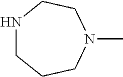 |
| 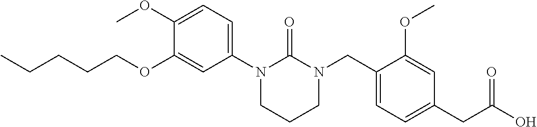 [SMILES: CCCCCOc1cc(N2CCCN(Cc3ccc(CC(=O)O)cc3OC)C2=O)ccc1OC] | 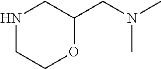 |
| 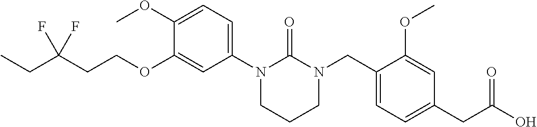 [SMILES: CCC(F)(F)CCOc1cc(N2CCCN(Cc3ccc(CC(=O)O)cc3OC)C2=O)ccc1OC] | 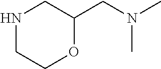 |
| 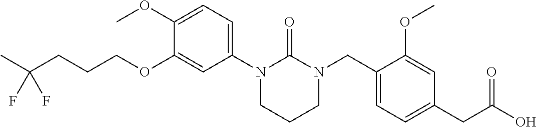 [SMILES: COc1cc(CC(=O)O)ccc1CN1CCCN(c2ccc(OC)c(OCCCC(C)(F)F)c2)C1=O] | 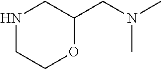 |
| 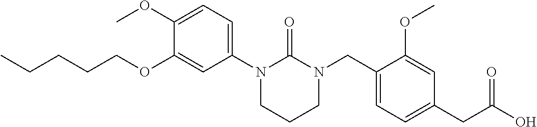 [SMILES: CCCCCOc1cc(N2CCCN(Cc3ccc(CC(=O)O)cc3OC)C2=O)ccc1OC] | 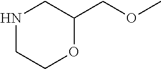 |
| 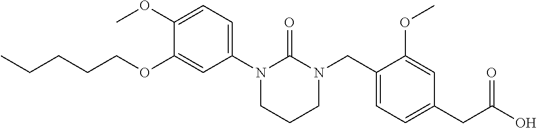 [SMILES: CCCCCOc1cc(N2CCCN(Cc3ccc(CC(=O)O)cc3OC)C2=O)ccc1OC] | 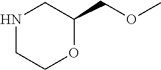 |
| 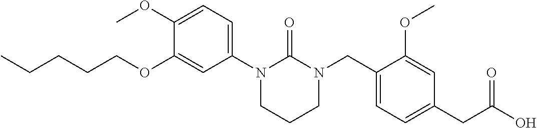 [SMILES: CCCCCOc1cc(N2CCCN(Cc3ccc(CC(=O)O)cc3OC)C2=O)ccc1OC] | 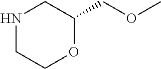 |
| 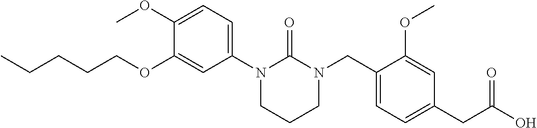 [SMILES: CCCCCOc1cc(N2CCCN(Cc3ccc(CC(=O)O)cc3OC)C2=O)ccc1OC] | 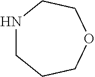 |
| 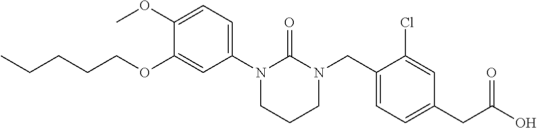 [SMILES: CCCCCOc1cc(N2CCCN(Cc3ccc(CC(=O)O)cc3Cl)C2=O)ccc1OC] | 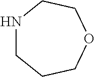 |
| 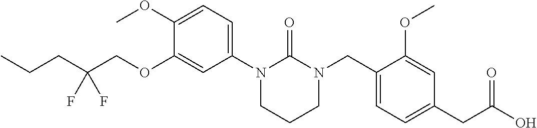 [SMILES: CCCC(F)(F)COc1cc(N2CCCN(Cc3ccc(CC(=O)O)cc3OC)C2=O)ccc1OC] | 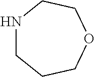 |
| 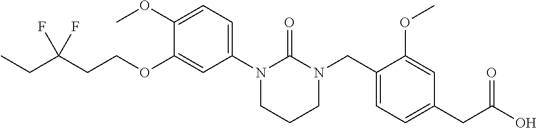 [SMILES: CCC(F)(F)CCOc1cc(N2CCCN(Cc3ccc(CC(=O)O)cc3OC)C2=O)ccc1OC] | 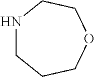 |
| 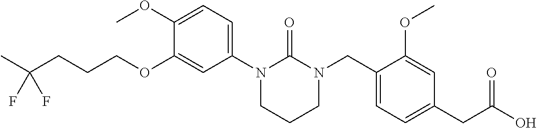 [SMILES: COc1cc(CC(=O)O)ccc1CN1CCCN(c2ccc(OC)c(OCCCC(C)(F)F)c2)C1=O] | 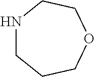 |
| 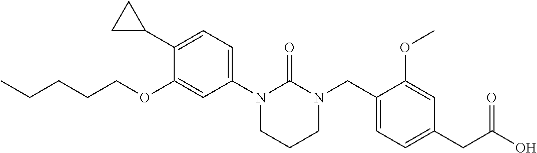 [SMILES: CCCCCOc1cc(N2CCCN(Cc3ccc(CC(=O)O)cc3OC)C2=O)ccc1C1CC1] | 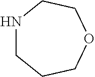 |

Compound

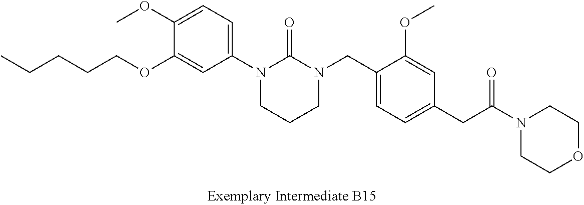 [SMILES: CCCCCOc1cc(N2CCCN(Cc3ccc(CC(=O)N4CCOCC4)cc3OC)C2=O)ccc1OC]

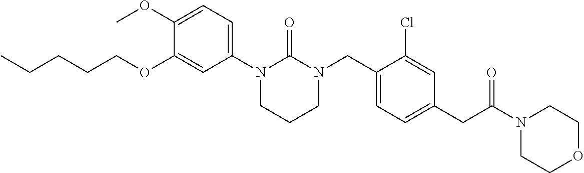 [SMILES: CCCCCOc1cc(N2CCCN(Cc3ccc(CC(=O)N4CCOCC4)cc3Cl)C2=O)ccc1OC]

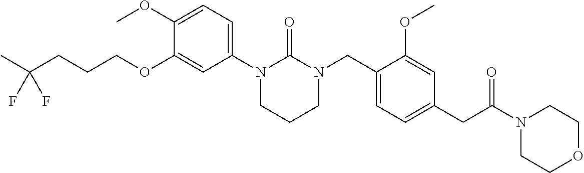 [SMILES: COc1cc(CC(=O)N2CCOCC2)ccc1CN1CCCN(c2ccc(OC)c(OCCCC(C)(F)F)c2)C1=O]

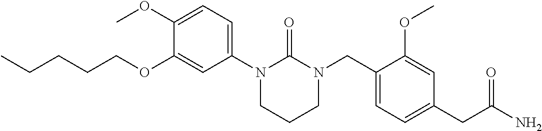 [SMILES: CCCCCOc1cc(N2CCCN(Cc3ccc(CC(N)=O)cc3OC)C2=O)ccc1OC]

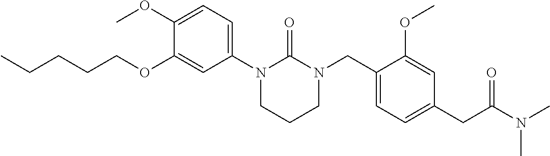 [SMILES: CCCCCOc1cc(N2CCCN(Cc3ccc(CC(=O)N(C)C)cc3OC)C2=O)ccc1OC]

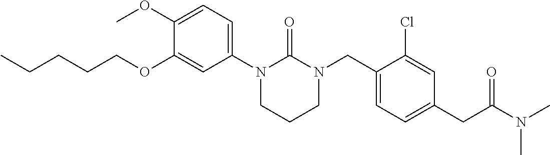 [SMILES: CCCCCOc1cc(N2CCCN(Cc3ccc(CC(=O)N(C)C)cc3Cl)C2=O)ccc1OC]

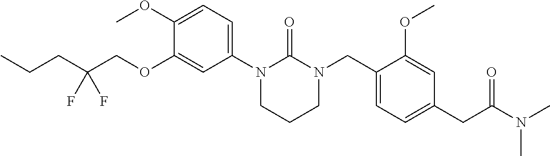 [SMILES: CCCC(F)(F)COc1cc(N2CCCN(Cc3ccc(CC(=O)N(C)C)cc3OC)C2=O)ccc1OC]

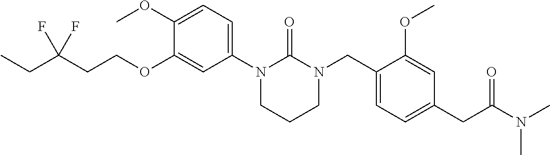 [SMILES: CCC(F)(F)CCOc1cc(N2CCCN(Cc3ccc(CC(=O)N(C)C)cc3OC)C2=O)ccc1OC]

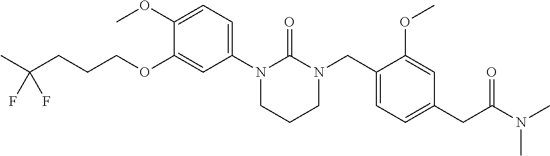 [SMILES: COc1cc(CC(=O)N(C)C)ccc1CN1CCCN(c2ccc(OC)c(OCCCC(C)(F)F)c2)C1=O]

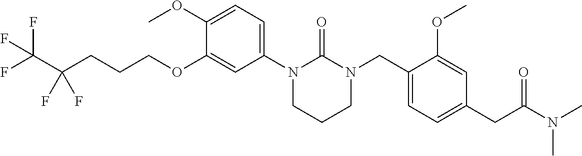 [SMILES: COc1cc(CC(=O)N(C)C)ccc1CN1CCCN(c2ccc(OC)c(OCCCC(F)(F)C(F)(F)F)c2)C1=O]

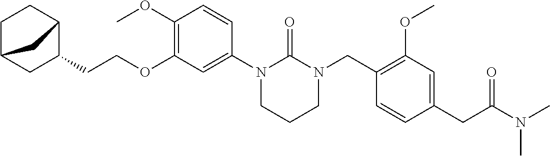 [SMILES: COc1cc(CC(=O)N(C)C)ccc1CN1CCCN(c2ccc(OC)c(OCC[C@@H]3C[C@@H]4CC[C@H]3C4)c2)C1=O]

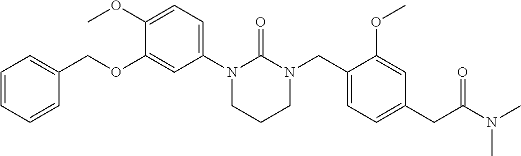 [SMILES: COc1cc(CC(=O)N(C)C)ccc1CN1CCCN(c2ccc(OC)c(OCc3ccccc3)c2)C1=O]

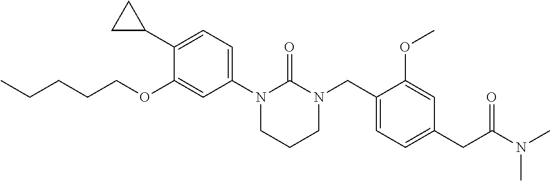 [SMILES: CCCCCOc1cc(N2CCCN(Cc3ccc(CC(=O)N(C)C)cc3OC)C2=O)ccc1C1CC1]

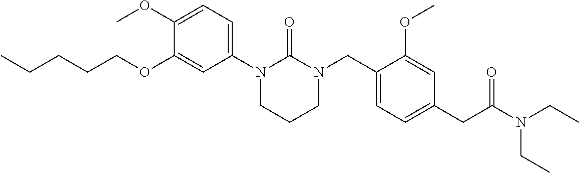 [SMILES: CCCCCOc1cc(N2CCCN(Cc3ccc(CC(=O)N(CC)CC)cc3OC)C2=O)ccc1OC]

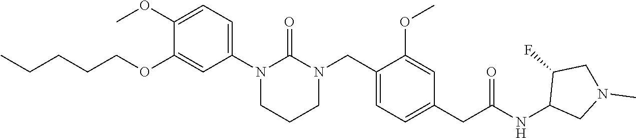 [SMILES: CCCCCOc1cc(N2CCCN(Cc3ccc(CC(=O)NC4CN(C)C[C@H]4F)cc3OC)C2=O)ccc1OC]

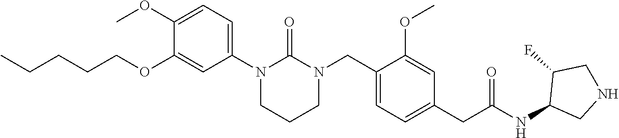 [SMILES: CCCCCOc1cc(N2CCCN(Cc3ccc(CC(=O)N[C@@H]4CNC[C@H]4F)cc3OC)C2=O)ccc1OC]

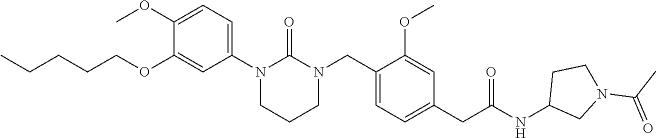 [SMILES: CCCCCOc1cc(N2CCCN(Cc3ccc(CC(=O)NC4CCN(C(C)=O)C4)cc3OC)C2=O)ccc1OC]

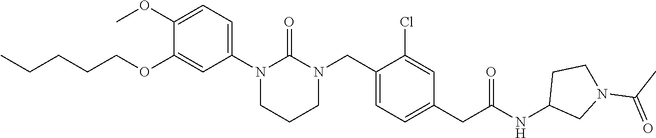 [SMILES: CCCCCOc1cc(N2CCCN(Cc3ccc(CC(=O)NC4CCN(C(C)=O)C4)cc3Cl)C2=O)ccc1OC]

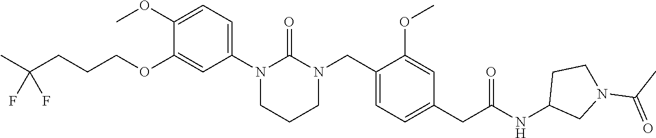 [SMILES: COc1cc(CC(=O)NC2CCN(C(C)=O)C2)ccc1CN1CCCN(c2ccc(OC)c(OCCCC(C)(F)F)c2)C1=O]

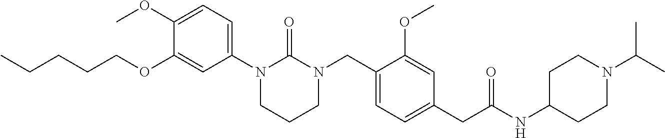 [SMILES: CCCCCOc1cc(N2CCCN(Cc3ccc(CC(=O)NC4CCN(C(C)C)CC4)cc3OC)C2=O)ccc1OC]

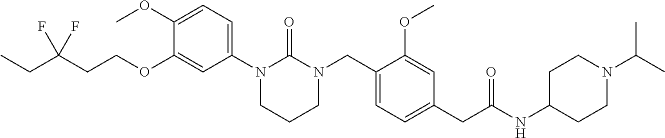 [SMILES: CCC(F)(F)CCOc1cc(N2CCCN(Cc3ccc(CC(=O)NC4CCN(C(C)C)CC4)cc3OC)C2=O)ccc1OC]

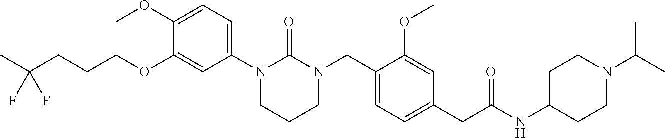 [SMILES: COc1cc(CC(=O)NC2CCN(C(C)C)CC2)ccc1CN1CCCN(c2ccc(OC)c(OCCCC(C)(F)F)c2)C1=O]

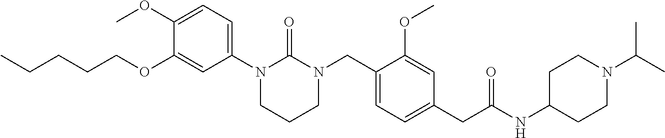 [SMILES: CCCCCOc1cc(N2CCCN(Cc3ccc(CC(=O)NC4CCN(C(C)C)CC4)cc3OC)C2=O)ccc1OC]

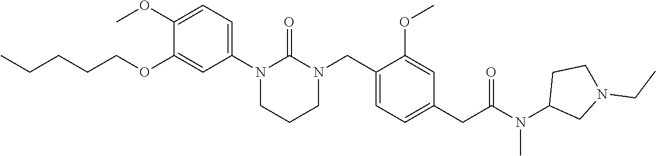 [SMILES: CCCCCOc1cc(N2CCCN(Cc3ccc(CC(=O)N(C)C4CCN(CC)C4)cc3OC)C2=O)ccc1OC]

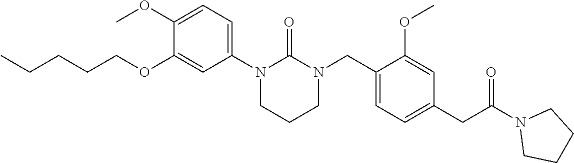 [SMILES: CCCCCOc1cc(N2CCCN(Cc3ccc(CC(=O)N4CCCC4)cc3OC)C2=O)ccc1OC]

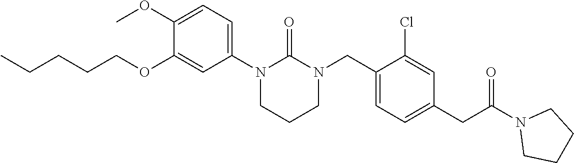 [SMILES: CCCCCOc1cc(N2CCCN(Cc3ccc(CC(=O)N4CCCC4)cc3Cl)C2=O)ccc1OC]

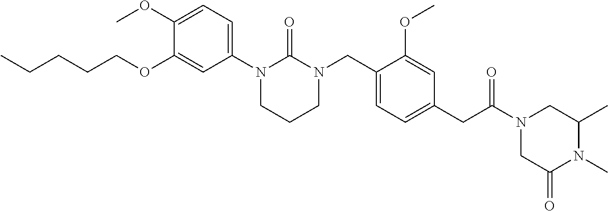 [SMILES: CCCCCOc1cc(N2CCCN(Cc3ccc(CC(=O)N4CC(=O)N(C)C(C)C4)cc3OC)C2=O)ccc1OC]

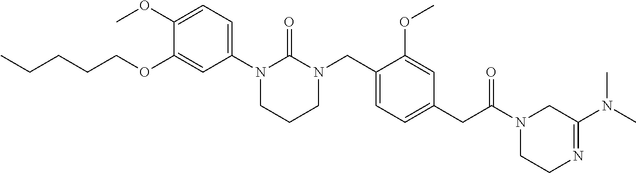 [SMILES: CCCCCOc1cc(N2CCCN(Cc3ccc(CC(=O)N4CCN=C(N(C)C)C4)cc3OC)C2=O)ccc1OC]

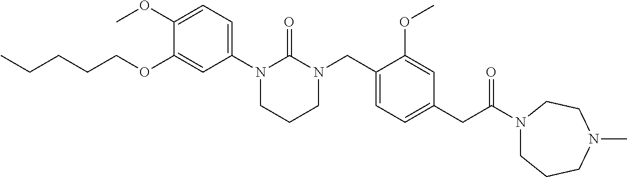 [SMILES: CCCCCOc1cc(N2CCCN(Cc3ccc(CC(=O)N4CCCN(C)CC4)cc3OC)C2=O)ccc1OC]

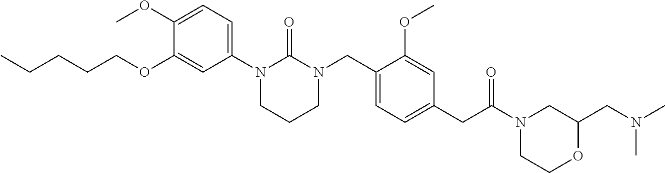 [SMILES: CCCCCOc1cc(N2CCCN(Cc3ccc(CC(=O)N4CCOC(CN(C)C)C4)cc3OC)C2=O)ccc1OC]

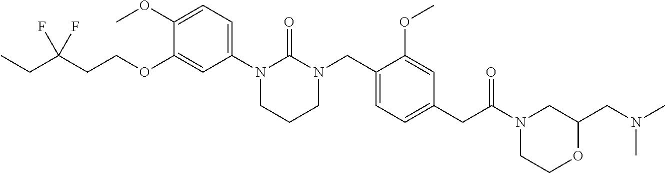 [SMILES: CCC(F)(F)CCOc1cc(N2CCCN(Cc3ccc(CC(=O)N4CCOC(CN(C)C)C4)cc3OC)C2=O)ccc1OC]

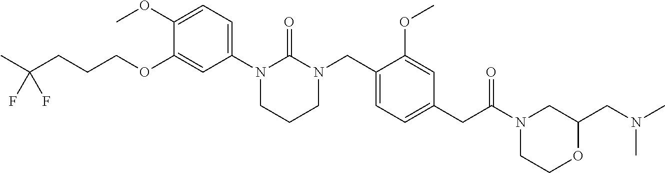 [SMILES: COc1cc(CC(=O)N2CCOC(CN(C)C)C2)ccc1CN1CCCN(c2ccc(OC)c(OCCCC(C)(F)F)c2)C1=O]

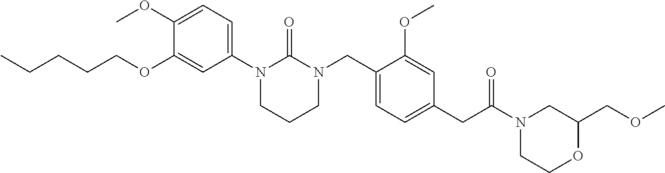 [SMILES: CCCCCOc1cc(N2CCCN(Cc3ccc(CC(=O)N4CCOC(COC)C4)cc3OC)C2=O)ccc1OC]

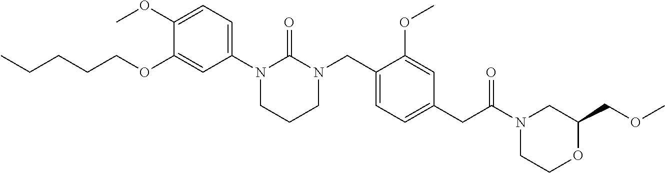 [SMILES: CCCCCOc1cc(N2CCCN(Cc3ccc(CC(=O)N4CCO[C@H](COC)C4)cc3OC)C2=O)ccc1OC]

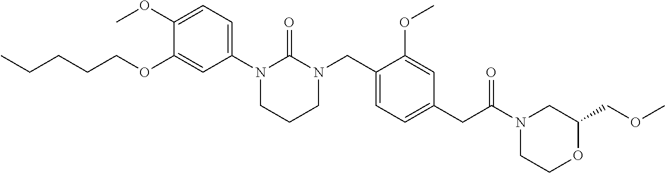 [SMILES: CCCCCOc1cc(N2CCCN(Cc3ccc(CC(=O)N4CCO[C@@H](COC)C4)cc3OC)C2=O)ccc1OC]

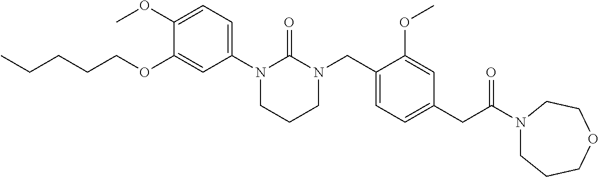 [SMILES: CCCCCOc1cc(N2CCCN(Cc3ccc(CC(=O)N4CCCOCC4)cc3OC)C2=O)ccc1OC]

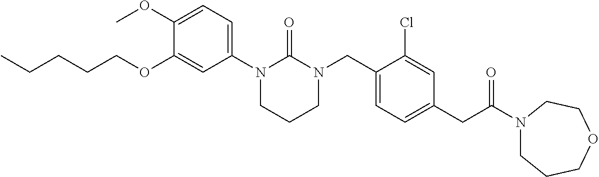 [SMILES: CCCCCOc1cc(N2CCCN(Cc3ccc(CC(=O)N4CCCOCC4)cc3Cl)C2=O)ccc1OC]

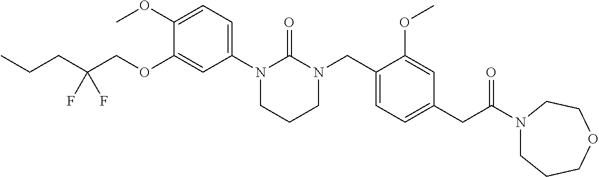 [SMILES: CCCC(F)(F)COc1cc(N2CCCN(Cc3ccc(CC(=O)N4CCCOCC4)cc3OC)C2=O)ccc1OC]

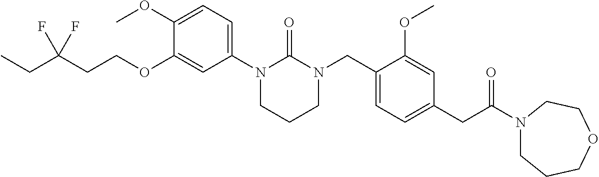 [SMILES: CCC(F)(F)CCOc1cc(N2CCCN(Cc3ccc(CC(=O)N4CCCOCC4)cc3OC)C2=O)ccc1OC]

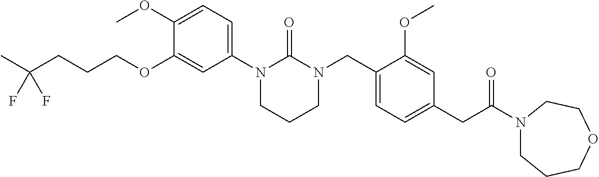 [SMILES: COc1cc(CC(=O)N2CCCOCC2)ccc1CN1CCCN(c2ccc(OC)c(OCCCC(C)(F)F)c2)C1=O]

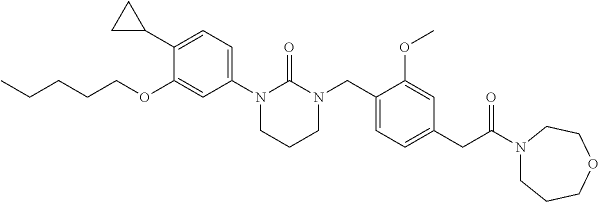 [SMILES: CCCCCOc1cc(N2CCCN(Cc3ccc(CC(=O)N4CCCOCC4)cc3OC)C2=O)ccc1C1CC1]

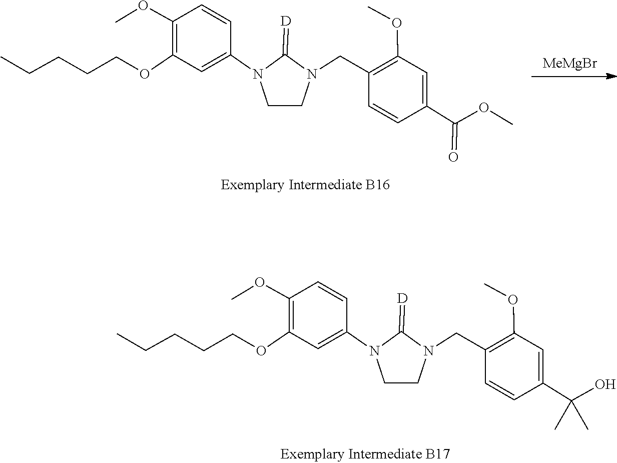

To a solution of Exemplary Intermediate B16 (methyl 3-methoxy-4-[[3-(4-methoxy-3-pentoxyphenyl)-2-oxoimidazolidin-1-yl]methyl]benzoate 100 mg, 0.22 mmol) in tetrahydrofuran (6 mL), cooled to −10° C., is added 3.0 M methylmagnesium bromide in diethyl ether (0.22 mL, 0.66 mmol) in a dropwise fashion. The reaction is stirred at room temperature for 3 h. Another 0.12 mL of 3.0 M methylmagnesium bromide in diethyl ether is added at 0° C., then the reaction is stirred at room temperature for another 1.5 h. Water is added at 0° C., and the mixture is extracted with ethyl acetate three times. The combined organic layers are dried over sodium sulfate, filtered and concentrated in vacuo. The residue is purified by column chromatography over silica gel (cyclohexane/ethyl acetate: 100/0 to 40/60) to afford Exemplary Intermediate B17 (1-[[4-(2-hydroxypropan-2-yl)-2-methoxyphenyl]methyl]-3-(4-methoxy-3-pentoxyphenyl)imidazolidin-2-one, 61 mg, 61% yield).

The following compounds are prepared in a similar manner:

Ester

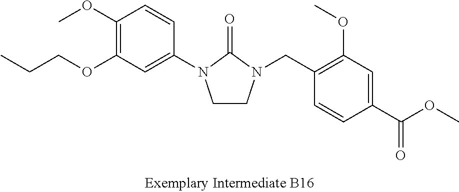 [SMILES: CCCOc1cc(N2CCN(Cc3ccc(C(=O)OC)cc3OC)C2=O)ccc1OC]

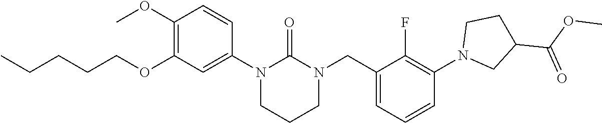 [SMILES: CCCCCOc1cc(N2CCCN(Cc3cccc(N4CCC(C(=O)OC)C4)c3F)C2=O)ccc1OC]

Compound

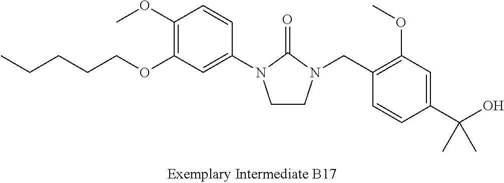 [SMILES: CCCCCOc1cc(N2CCN(Cc3ccc(C(C)(C)O)cc3OC)C2=O)ccc1OC]

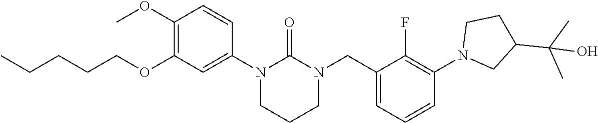 [SMILES: CCCCCOc1cc(N2CCCN(Cc3cccc(N4CCC(C(C)(C)O)C4)c3F)C2=O)ccc1OC]

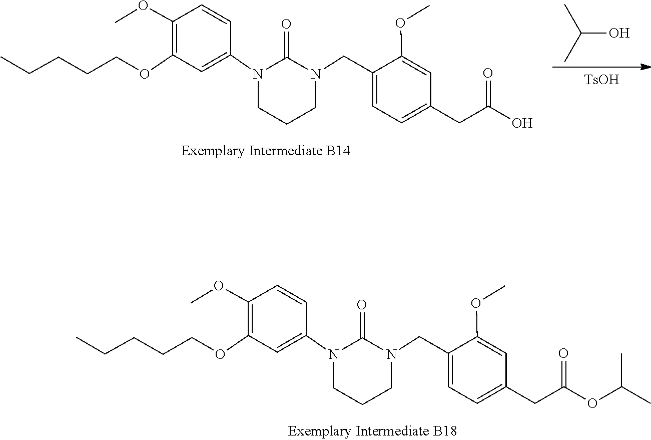

Exemplary Intermediate B14 (2-(3-methoxy-4-((3-(4-methoxy-3-(pentyloxy)phenyl)-2-oxotetrahydropyrimidin-1(2H)-yl)methyl)phenyl)acetic acid) is reacted with isopropanol in the presence of tosic acid. The reaction affords Exemplary Intermediate B18 (isopropyl 2-(3-methoxy-4-((3-(4-methoxy-3-(pentyloxy)phenyl)-2-oxotetrahydropyrimidin-1(2H)-yl)methyl)phenyl)acetate).

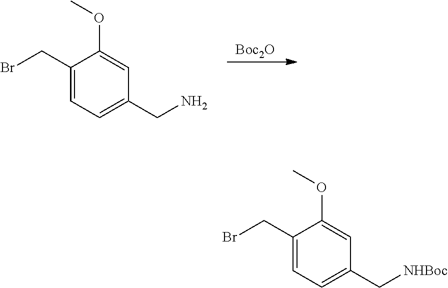 [SMILES: "Boc2O" ; COc1cc(CN)ccc1CBr ; COc1cc(CNC(=O)OC(C)(C)C)ccc1CBr]

To a solution of (4-(bromomethyl)-3-methoxyphenyl)methanamine in dichloromethane is added triethylamine and di-tert-butyl dicarbonate (4.5 g, 20.6 mmol). The reaction is stirred at room temperature for 1.5 h. The mixture is diluted with dichloromethane and washed with saturated aqueous ammonium chloride solution. Organic phase is dried over a phase separator and concentrated in vacuo. The residue is purified by column chromatography over silica gel (cyclohexane/ethyl acetate: 100/0 to 85/15) to afford tert-butyl (4-(bromomethyl)-3-methoxybenzyl)carbamate.

The following compounds are prepared in a similar manner:

| Amine | Compound |
|---|---|
| 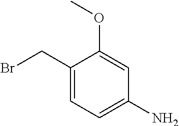 | 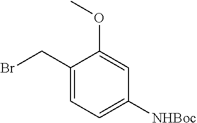 [SMILES: COc1cc(NC(=O)OC(C)(C)C)ccc1CBr] |
| 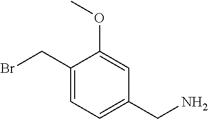 | 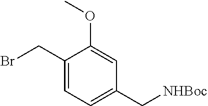 [SMILES: COc1cc(CNC(=O)OC(C)(C)C)ccc1CBr] |
| 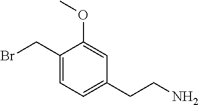 | 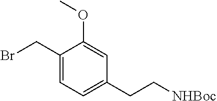 [SMILES: COc1cc(CCNC(=O)OC(C)(C)C)ccc1CBr] |
| 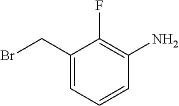 | 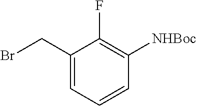 [SMILES: CC(C)(C)OC(=O)Nc1cccc(CBr)c1F] |
| 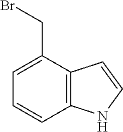 [SMILES: BrCc1cccc2[nH]ccc12] | 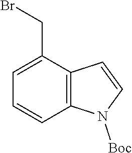 [SMILES: CC(C)(C)OC(=O)n1ccc2c(CBr)cccc21] |
| 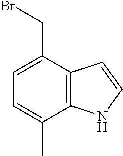 | 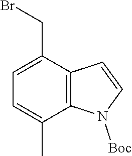 [SMILES: Cc1ccc(CBr)c2ccn(C(=O)OC(C)(C)C)c12] |
| 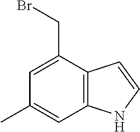 | 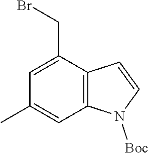 [SMILES: Cc1cc(CBr)c2ccn(C(=O)OC(C)(C)C)c2c1] |
| 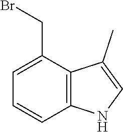 | 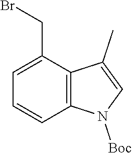 [SMILES: Cc1cn(C(=O)OC(C)(C)C)c2cccc(CBr)c12] |
| 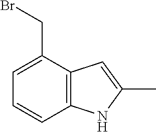 | 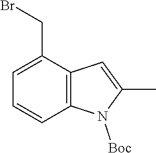 [SMILES: Cc1cc2c(CBr)cccc2n1C(=O)OC(C)(C)C] |
| 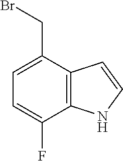 [SMILES: Fc1ccc(CBr)c2cc[nH]c12] | 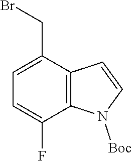 [SMILES: CC(C)(C)OC(=O)n1ccc2c(CBr)ccc(F)c21] |
| 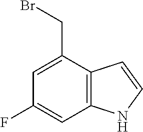 [SMILES: Fc1cc(CBr)c2cc[nH]c2c1] | 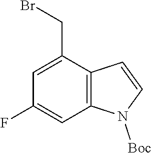 [SMILES: CC(C)(C)OC(=O)n1ccc2c(CBr)cc(F)cc21] |
| 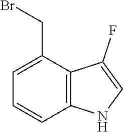 [SMILES: Fc1c[nH]c2cccc(CBr)c12] | 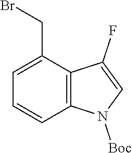 [SMILES: CC(C)(C)OC(=O)n1cc(F)c2c(CBr)cccc21] |
| 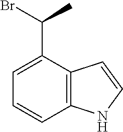 | 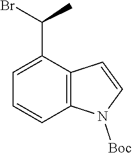 [SMILES: C[C@H](Br)c1cccc2c1ccn2C(=O)OC(C)(C)C] |
| 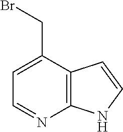 [SMILES: BrCc1ccnc2[nH]ccc12] | 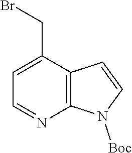 [SMILES: CC(C)(C)OC(=O)n1ccc2c(CBr)ccnc21] |
| 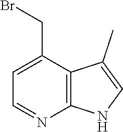 | 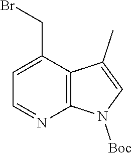 [SMILES: Cc1cn(C(=O)OC(C)(C)C)c2nccc(CBr)c12] |
| 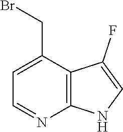 [SMILES: Fc1c[nH]c2nccc(CBr)c12] | 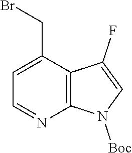 [SMILES: CC(C)(C)OC(=O)n1cc(F)c2c(CBr)ccnc21] |
| 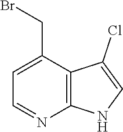 [SMILES: Clc1c[nH]c2nccc(CBr)c12] | 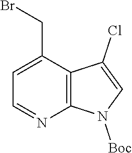 [SMILES: CC(C)(C)OC(=O)n1cc(Cl)c2c(CBr)ccnc21] |
| 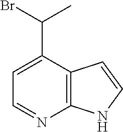 | 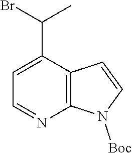 [SMILES: CC(Br)c1ccnc2c1ccn2C(=O)OC(C)(C)C] |
| 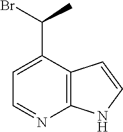 | 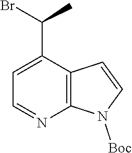 [SMILES: C[C@H](Br)c1ccnc2c1ccn2C(=O)OC(C)(C)C] |
| 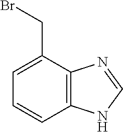 [SMILES: BrCc1cccc2[nH]cnc12] | 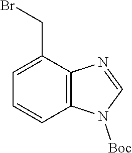 [SMILES: CC(C)(C)OC(=O)n1cnc2c(CBr)cccc21] |
| 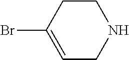 | 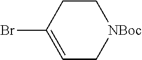 [SMILES: CC(C)(C)OC(=O)N1CC=C(Br)CC1] |

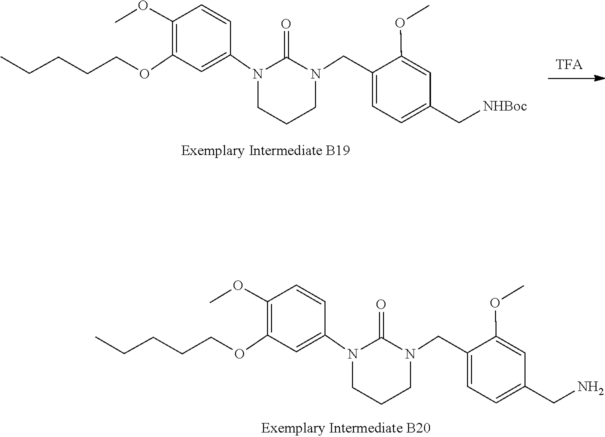 [SMILES: CCCCCOc1cc(N2CCCN(Cc3ccc(CNC(=O)OC(C)(C)C)cc3OC)C2=O)ccc1OC ; CCCCCOc1cc(N2CCCN(Cc3ccc(CN)cc3OC)C2=O)ccc1OC]

Exemplary Intermediate B19 (tert-butyl (3-methoxy-4-((3-(4-methoxy-3-(pentyloxy)phenyl)-2-oxotetrahydropyrimidin-1(2H)-yl)methyl)benzyl)carbamate) is dissolved in dichloromethane and cooled to 0° C. Neat trifluoroacetic acid is added dropwise over 5 min. The mixture is stirred for 2 h at 0° C., then concentrated in vacuo. The residue is suspended in ethyl acetate (100 mL) and filtered. The solids are dried to afford Exemplary Intermediate B20 (1-(4-(aminomethyl)-2-methoxybenzyl)-3-(4-methoxy-3-(pentyloxy)phenyl)tetrahydropyrimidin-2(1H)-one).

The following compounds are prepared in a similar manner as Exemplary Intermediate B20, as described above.

Cyclic Urea

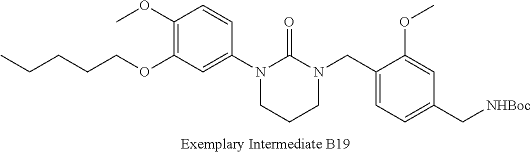 [SMILES: CCCCCOc1cc(N2CCCN(Cc3ccc(CNC(=O)OC(C)(C)C)cc3OC)C2=O)ccc1OC]

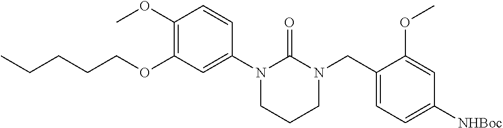 [SMILES: CCCCCOc1cc(N2CCCN(Cc3ccc(NC(=O)OC(C)(C)C)cc3OC)C2=O)ccc1OC]

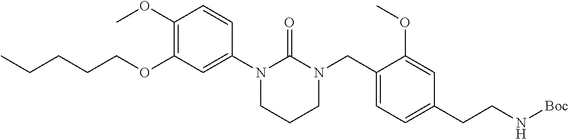 [SMILES: CCCCCOc1cc(N2CCCN(Cc3ccc(CCNC(=O)OC(C)(C)C)cc3OC)C2=O)ccc1OC]

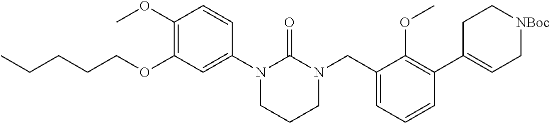 [SMILES: CCCCCOc1cc(N2CCCN(Cc3cccc(C4=CCN(C(=O)OC(C)(C)C)CC4)c3OC)C2=O)ccc1OC]

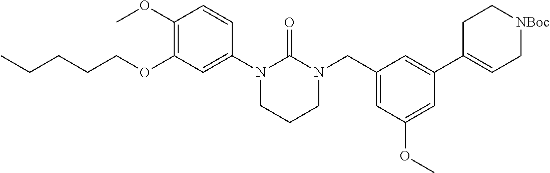 [SMILES: CCCCCOc1cc(N2CCCN(Cc3cc(OC)cc(C4=CCN(C(=O)OC(C)(C)C)CC4)c3)C2=O)ccc1OC]

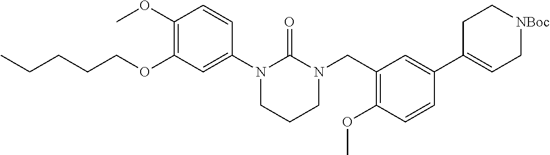 [SMILES: CCCCCOc1cc(N2CCCN(Cc3cc(C4=CCN(C(=O)OC(C)(C)C)CC4)ccc3OC)C2=O)ccc1OC]

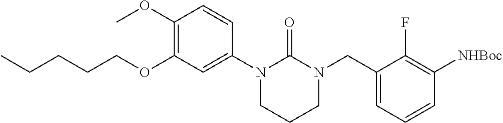 [SMILES: CCCCCOc1cc(N2CCCN(Cc3cccc(NC(=O)OC(C)(C)C)c3F)C2=O)ccc1OC]

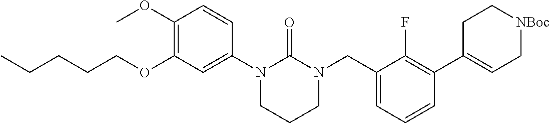 [SMILES: CCCCCOc1cc(N2CCCN(Cc3cccc(C4=CCN(C(=O)OC(C)(C)C)CC4)c3F)C2=O)ccc1OC]

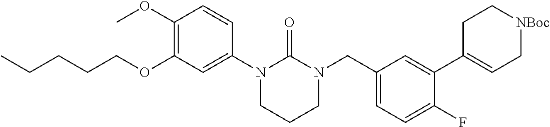 [SMILES: CCCCCOc1cc(N2CCCN(Cc3ccc(F)c(C4=CCN(C(=O)OC(C)(C)C)CC4)c3)C2=O)ccc1OC]

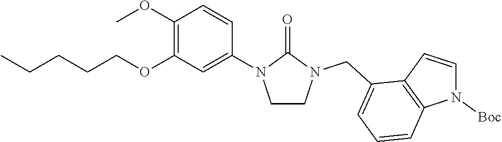 [SMILES: CCCCCOc1cc(N2CCN(Cc3cccc4c3ccn4C(=O)OC(C)(C)C)C2=O)ccc1OC]

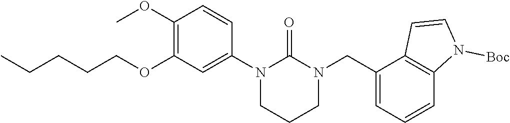 [SMILES: CCCCCOc1cc(N2CCCN(Cc3cccc4c3ccn4C(=O)OC(C)(C)C)C2=O)ccc1OC]

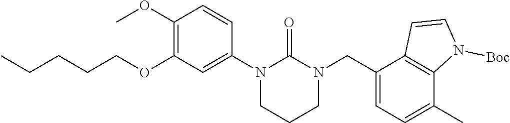 [SMILES: CCCCCOc1cc(N2CCCN(Cc3ccc(C)c4c3ccn4C(=O)OC(C)(C)C)C2=O)ccc1OC]

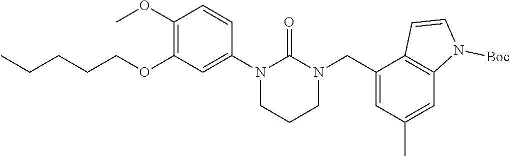 [SMILES: CCCCCOc1cc(N2CCCN(Cc3cc(C)cc4c3ccn4C(=O)OC(C)(C)C)C2=O)ccc1OC]

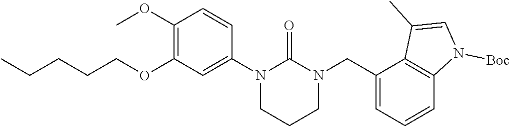 [SMILES: CCCCCOc1cc(N2CCCN(Cc3cccc4c3c(C)cn4C(=O)OC(C)(C)C)C2=O)ccc1OC]

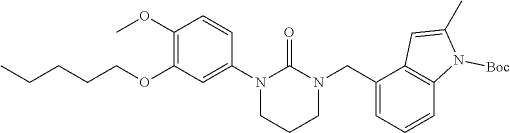 [SMILES: CCCCCOc1cc(N2CCCN(Cc3cccc4c3cc(C)n4C(=O)OC(C)(C)C)C2=O)ccc1OC]

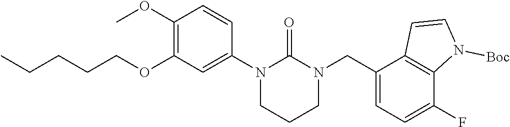 [SMILES: CCCCCOc1cc(N2CCCN(Cc3ccc(F)c4c3ccn4C(=O)OC(C)(C)C)C2=O)ccc1OC]

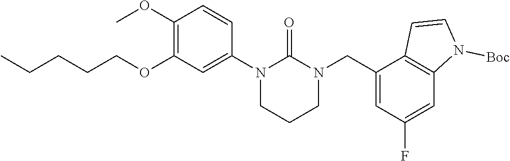 [SMILES: CCCCCOc1cc(N2CCCN(Cc3cc(F)cc4c3ccn4C(=O)OC(C)(C)C)C2=O)ccc1OC]

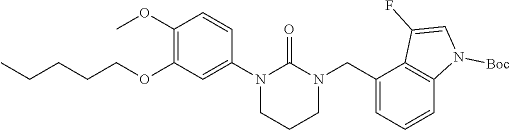 [SMILES: CCCCCOc1cc(N2CCCN(Cc3cccc4c3c(F)cn4C(=O)OC(C)(C)C)C2=O)ccc1OC]

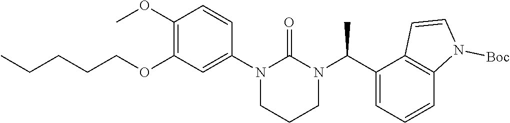 [SMILES: CCCCCOc1cc(N2CCCN([C@@H](C)c3cccc4c3ccn4C(=O)OC(C)(C)C)C2=O)ccc1OC]

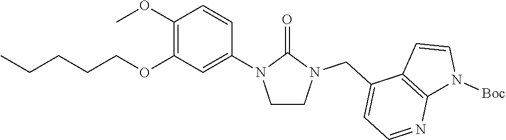 [SMILES: CCCCCOc1cc(N2CCN(Cc3ccnc4c3ccn4C(=O)OC(C)(C)C)C2=O)ccc1OC]

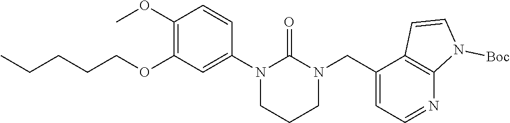 [SMILES: CCCCCOc1cc(N2CCCN(Cc3ccnc4c3ccn4C(=O)OC(C)(C)C)C2=O)ccc1OC]

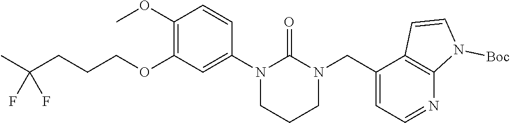 [SMILES: COc1ccc(N2CCCN(Cc3ccnc4c3ccn4C(=O)OC(C)(C)C)C2=O)cc1OCCCC(C)(F)F]

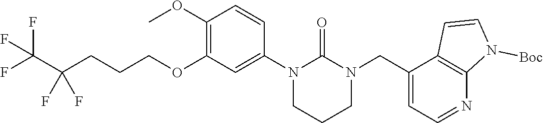 [SMILES: COc1ccc(N2CCCN(Cc3ccnc4c3ccn4C(=O)OC(C)(C)C)C2=O)cc1OCCCC(F)(F)C(F)(F)F]

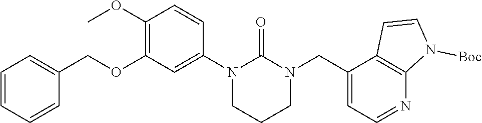 [SMILES: COc1ccc(N2CCCN(Cc3ccnc4c3ccn4C(=O)OC(C)(C)C)C2=O)cc1OCc1ccccc1]

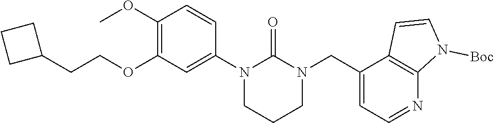 [SMILES: COc1ccc(N2CCCN(Cc3ccnc4c3ccn4C(=O)OC(C)(C)C)C2=O)cc1OCCC1CCC1]

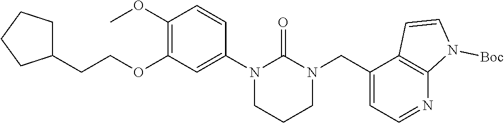 [SMILES: COc1ccc(N2CCCN(Cc3ccnc4c3ccn4C(=O)OC(C)(C)C)C2=O)cc1OCCC1CCCC1]

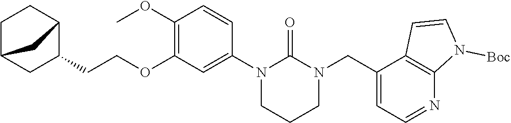 [SMILES: COc1ccc(N2CCCN(Cc3ccnc4c3ccn4C(=O)OC(C)(C)C)C2=O)cc1OCC[C@@H]1C[C@@H]2CC[C@H]1C2]

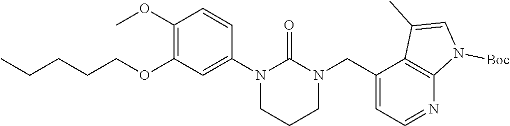 [SMILES: CCCCCOc1cc(N2CCCN(Cc3ccnc4c3c(C)cn4C(=O)OC(C)(C)C)C2=O)ccc1OC]

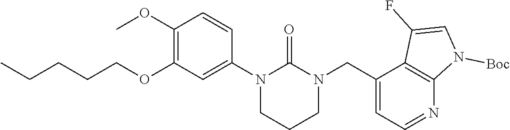 [SMILES: CCCCCOc1cc(N2CCCN(Cc3ccnc4c3c(F)cn4C(=O)OC(C)(C)C)C2=O)ccc1OC]

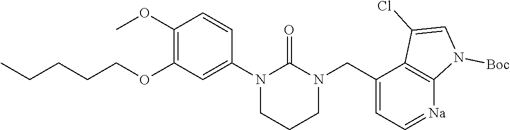

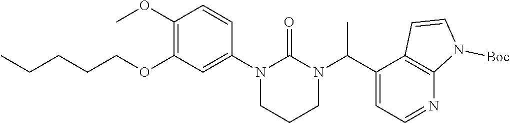 [SMILES: CCCCCOc1cc(N2CCCN(C(C)c3ccnc4c3ccn4C(=O)OC(C)(C)C)C2=O)ccc1OC]

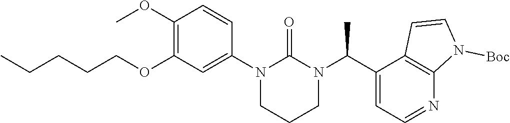 [SMILES: CCCCCOc1cc(N2CCCN([C@@H](C)c3ccnc4c3ccn4C(=O)OC(C)(C)C)C2=O)ccc1OC]

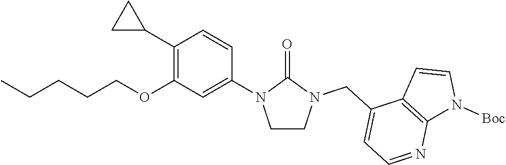 [SMILES: CCCCCOc1cc(N2CCN(Cc3ccnc4c3ccn4C(=O)OC(C)(C)C)C2=O)ccc1C1CC1]

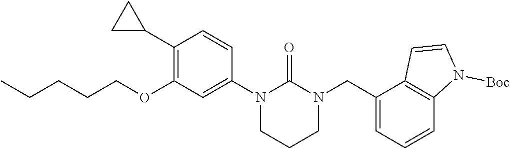 [SMILES: CCCCCOc1cc(N2CCCN(Cc3cccc4c3ccn4C(=O)OC(C)(C)C)C2=O)ccc1C1CC1]

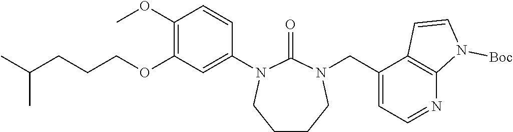 [SMILES: COc1ccc(N2CCCCN(Cc3ccnc4c3ccn4C(=O)OC(C)(C)C)C2=O)cc1OCCCC(C)C]

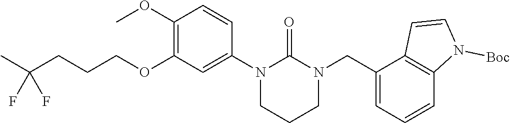 [SMILES: COc1ccc(N2CCCN(Cc3cccc4c3ccn4C(=O)OC(C)(C)C)C2=O)cc1OCCCC(C)(F)F]

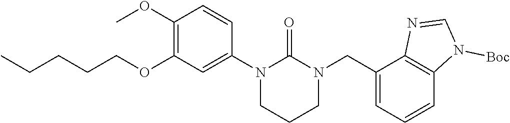 [SMILES: CCCCCOc1cc(N2CCCN(Cc3cccc4c3ncn4C(=O)OC(C)(C)C)C2=O)ccc1OC]

Compound

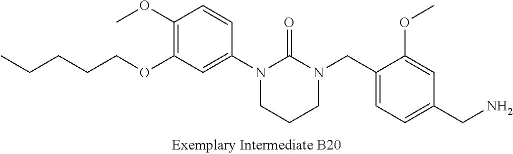 [SMILES: CCCCCOc1cc(N2CCCN(Cc3ccc(CN)cc3OC)C2=O)ccc1OC]

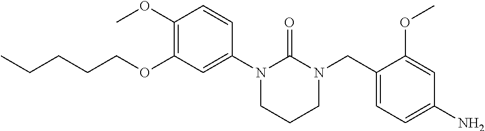 [SMILES: CCCCCOc1cc(N2CCCN(Cc3ccc(N)cc3OC)C2=O)ccc1OC]

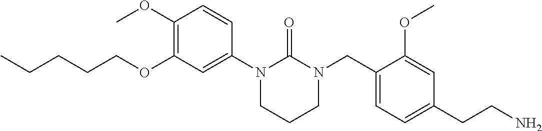 [SMILES: CCCCCOc1cc(N2CCCN(Cc3ccc(CCN)cc3OC)C2=O)ccc1OC]

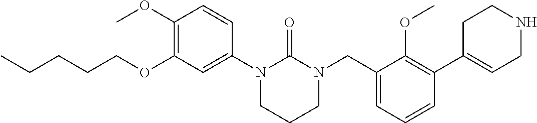 [SMILES: CCCCCOc1cc(N2CCCN(Cc3cccc(C4=CCNCC4)c3OC)C2=O)ccc1OC]

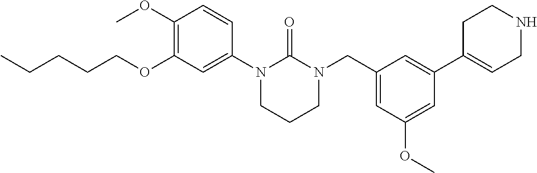 [SMILES: CCCCCOc1cc(N2CCCN(Cc3cc(OC)cc(C4=CCNCC4)c3)C2=O)ccc1OC]

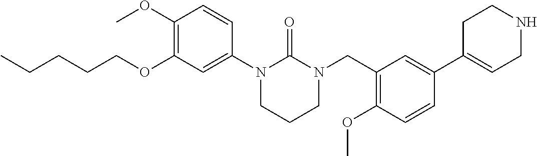 [SMILES: CCCCCOc1cc(N2CCCN(Cc3cc(C4=CCNCC4)ccc3OC)C2=O)ccc1OC]

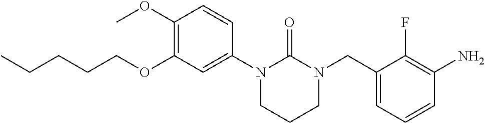 [SMILES: CCCCCOc1cc(N2CCCN(Cc3cccc(N)c3F)C2=O)ccc1OC]

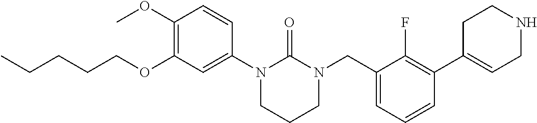 [SMILES: CCCCCOc1cc(N2CCCN(Cc3cccc(C4=CCNCC4)c3F)C2=O)ccc1OC]

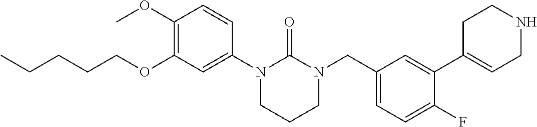 [SMILES: CCCCCOc1cc(N2CCCN(Cc3ccc(F)c(C4=CCNCC4)c3)C2=O)ccc1OC]

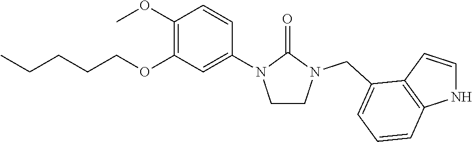 [SMILES: CCCCCOc1cc(N2CCN(Cc3cccc4[nH]ccc34)C2=O)ccc1OC]

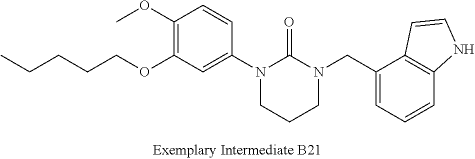 [SMILES: CCCCCOc1cc(N2CCCN(Cc3cccc4[nH]ccc34)C2=O)ccc1OC]

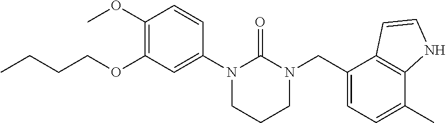 [SMILES: CCCCOc1cc(N2CCCN(Cc3ccc(C)c4[nH]ccc34)C2=O)ccc1OC]

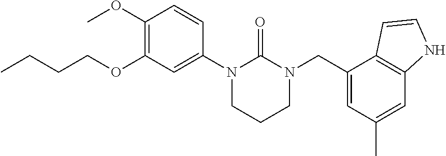 [SMILES: CCCCOc1cc(N2CCCN(Cc3cc(C)cc4[nH]ccc34)C2=O)ccc1OC]

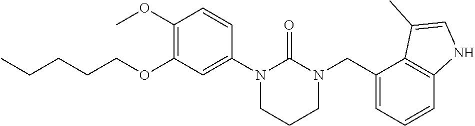 [SMILES: CCCCCOc1cc(N2CCCN(Cc3cccc4[nH]cc(C)c34)C2=O)ccc1OC]

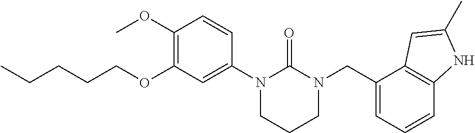 [SMILES: CCCCCOc1cc(N2CCCN(Cc3cccc4[nH]c(C)cc34)C2=O)ccc1OC]

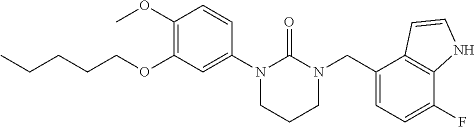 [SMILES: CCCCCOc1cc(N2CCCN(Cc3ccc(F)c4[nH]ccc34)C2=O)ccc1OC]

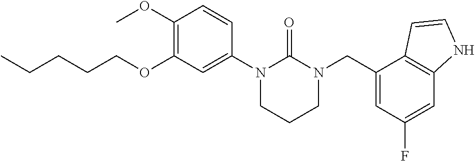 [SMILES: CCCCCOc1cc(N2CCCN(Cc3cc(F)cc4[nH]ccc34)C2=O)ccc1OC]

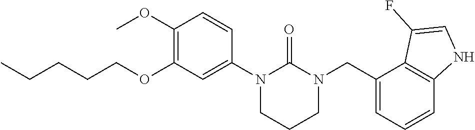 [SMILES: CCCCCOc1cc(N2CCCN(Cc3cccc4[nH]cc(F)c34)C2=O)ccc1OC]

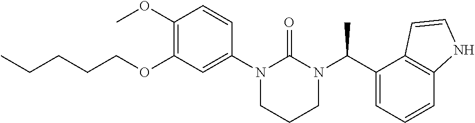 [SMILES: CCCCCOc1cc(N2CCCN([C@@H](C)c3cccc4[nH]ccc34)C2=O)ccc1OC]

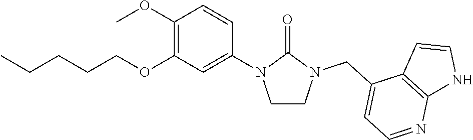 [SMILES: CCCCCOc1cc(N2CCN(Cc3ccnc4[nH]ccc34)C2=O)ccc1OC]

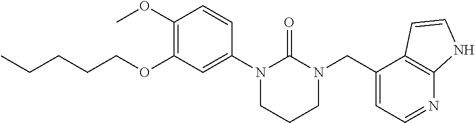 [SMILES: CCCCCOc1cc(N2CCCN(Cc3ccnc4[nH]ccc34)C2=O)ccc1OC]

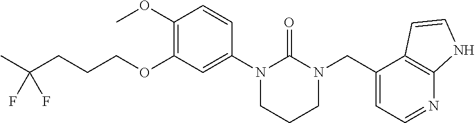 [SMILES: COc1ccc(N2CCCN(Cc3ccnc4[nH]ccc34)C2=O)cc1OCCCC(C)(F)F]

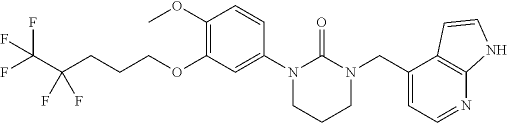 [SMILES: COc1ccc(N2CCCN(Cc3ccnc4[nH]ccc34)C2=O)cc1OCCCC(F)(F)C(F)(F)F]

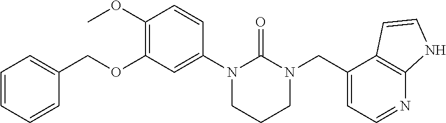 [SMILES: COc1ccc(N2CCCN(Cc3ccnc4[nH]ccc34)C2=O)cc1OCc1ccccc1]

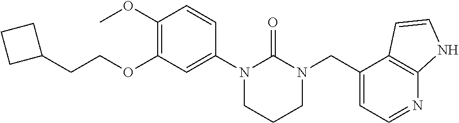 [SMILES: COc1ccc(N2CCCN(Cc3ccnc4[nH]ccc34)C2=O)cc1OCCC1CCC1]

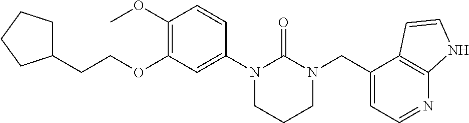 [SMILES: COc1ccc(N2CCCN(Cc3ccnc4[nH]ccc34)C2=O)cc1OCCC1CCCC1]

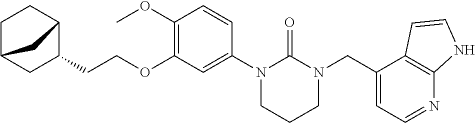 [SMILES: COc1ccc(N2CCCN(Cc3ccnc4[nH]ccc34)C2=O)cc1OCC[C@@H]1C[C@@H]2CC[C@H]1C2]

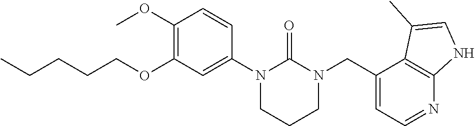 [SMILES: CCCCCOc1cc(N2CCCN(Cc3ccnc4[nH]cc(C)c34)C2=O)ccc1OC]

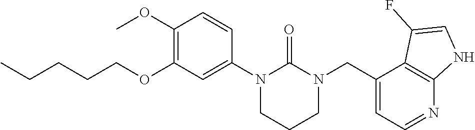 [SMILES: CCCCCOc1cc(N2CCCN(Cc3ccnc4[nH]cc(F)c34)C2=O)ccc1OC]

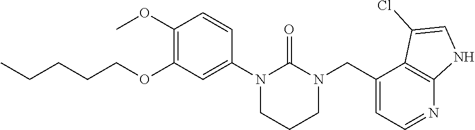 [SMILES: CCCCCOc1cc(N2CCCN(Cc3ccnc4[nH]cc(Cl)c34)C2=O)ccc1OC]

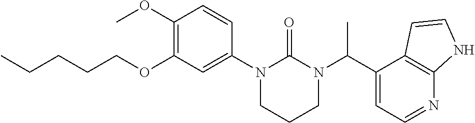 [SMILES: CCCCCOc1cc(N2CCCN(C(C)c3ccnc4[nH]ccc34)C2=O)ccc1OC]

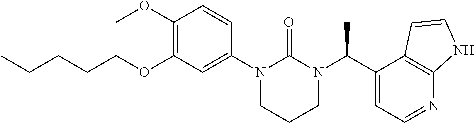 [SMILES: CCCCCOc1cc(N2CCCN([C@@H](C)c3ccnc4[nH]ccc34)C2=O)ccc1OC]

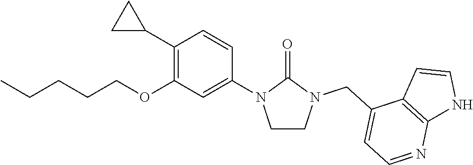 [SMILES: CCCCCOc1cc(N2CCN(Cc3ccnc4[nH]ccc34)C2=O)ccc1C1CC1]

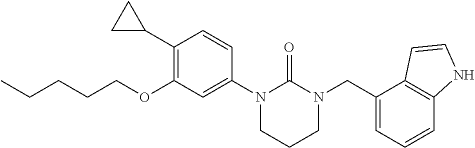 [SMILES: CCCCCOc1cc(N2CCCN(Cc3cccc4[nH]ccc34)C2=O)ccc1C1CC1]

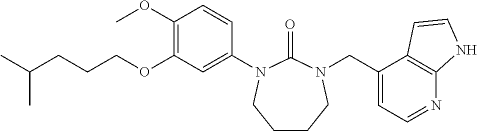 [SMILES: COc1ccc(N2CCCCN(Cc3ccnc4[nH]ccc34)C2=O)cc1OCCCC(C)C]

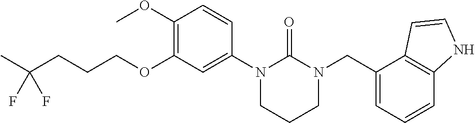 [SMILES: COc1ccc(N2CCCN(Cc3cccc4[nH]ccc34)C2=O)cc1OCCCC(C)(F)F]

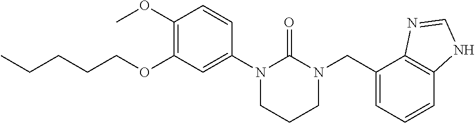 [SMILES: CCCCCOc1cc(N2CCCN(Cc3cccc4[nH]cnc34)C2=O)ccc1OC]

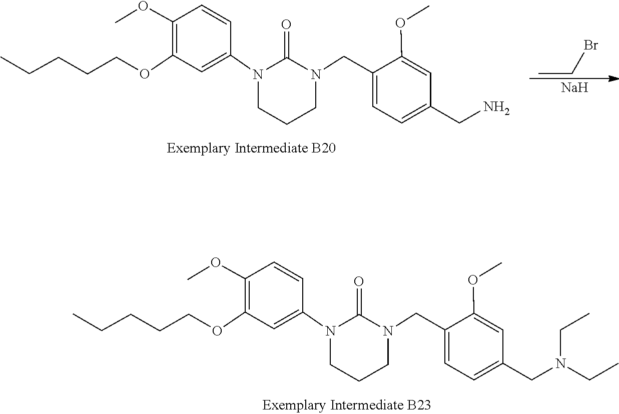

Exemplary Intermediate B20 (1-(4-aminomethyl)-2-methoxybenzyl)-3-(4-methoxy-3-(pentyloxy)phenyl)tetrahydropyrimidin-2(1H)-one) is combined with ethyl bromide and sodium hydride. The resulting reaction affords Exemplary Intermediate B23 (1-(4-((diethylamino) methyl)-2-methoxybenzyl)-3-(4-methoxy-3-(pentyloxy)phenyl)tetrahydropyrimidin-2(1H)-one).

The following compounds are prepared in a similar manner as Exemplary Intermediate B23, as described above.

| Amine | Bromide |
|---|---|
| 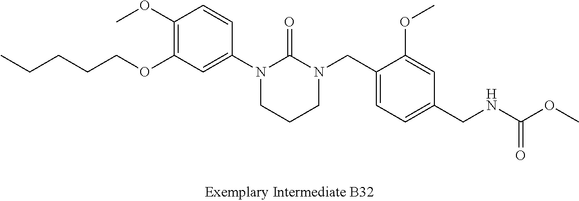 [SMILES: CCCCCOc1cc(N2CCCN(Cc3ccc(CNC(=O)OC)cc3OC)C2=O)ccc1OC] | MeI |
| 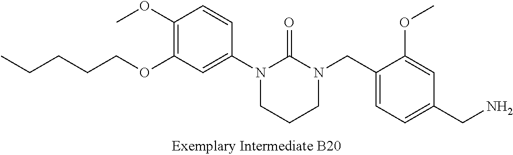 [SMILES: CCCCCOc1cc(N2CCCN(Cc3ccc(CN)cc3OC)C2=O)ccc1OC] | 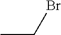 |
| 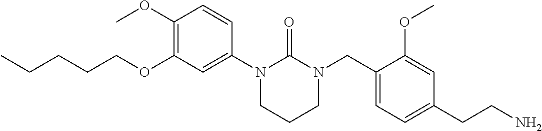 [SMILES: CCCCCOc1cc(N2CCCN(Cc3ccc(CCN)cc3OC)C2=O)ccc1OC] | 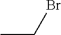 |
| 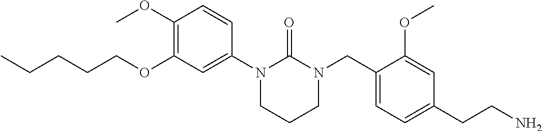 [SMILES: CCCCCOc1cc(N2CCCN(Cc3ccc(CCN)cc3OC)C2=O)ccc1OC] | 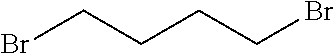 |
| 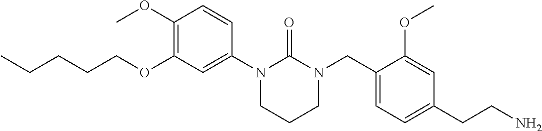 [SMILES: CCCCCOc1cc(N2CCCN(Cc3ccc(CCN)cc3OC)C2=O)ccc1OC] | 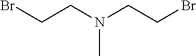 |
| 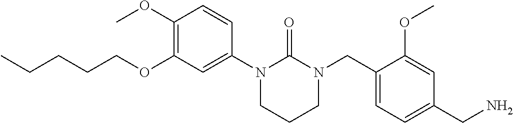 [SMILES: CCCCCOc1cc(N2CCCN(Cc3ccc(CN)cc3OC)C2=O)ccc1OC] | 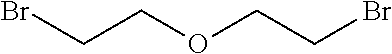 |
| 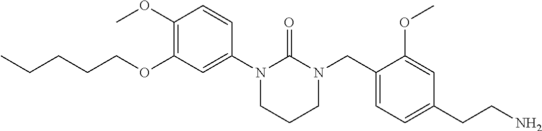 [SMILES: CCCCCOc1cc(N2CCCN(Cc3ccc(CCN)cc3OC)C2=O)ccc1OC] | 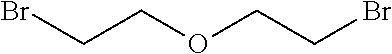 |
| 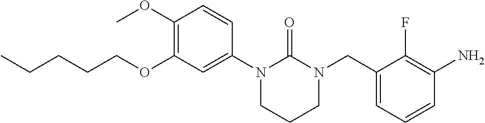 [SMILES: CCCCCOc1cc(N2CCCN(Cc3cccc(N)c3F)C2=O)ccc1OC] | 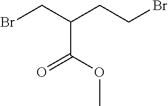 |
| 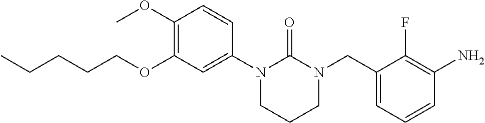 [SMILES: CCCCCOc1cc(N2CCCN(Cc3cccc(N)c3F)C2=O)ccc1OC] | 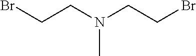 |
| 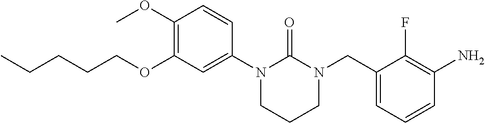 [SMILES: CCCCCOc1cc(N2CCCN(Cc3cccc(N)c3F)C2=O)ccc1OC] | 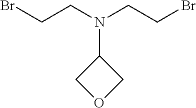 [SMILES: BrCCN(CCBr)C1COC1] |
| 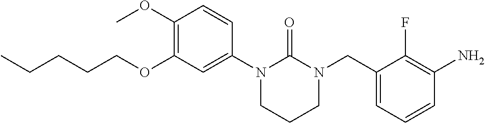 [SMILES: CCCCCOc1cc(N2CCCN(Cc3cccc(N)c3F)C2=O)ccc1OC] | 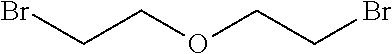 |
| 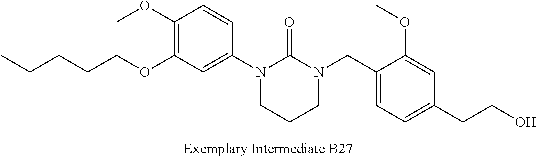 [SMILES: CCCCCOc1cc(N2CCCN(Cc3ccc(CCO)cc3OC)C2=O)ccc1OC] | MeI |
| 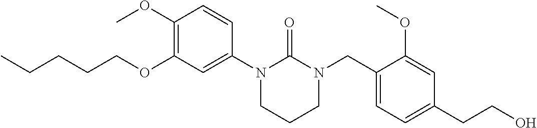 [SMILES: CCCCCOc1cc(N2CCCN(Cc3ccc(CCO)cc3OC)C2=O)ccc1OC] | 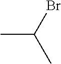 |

Compound

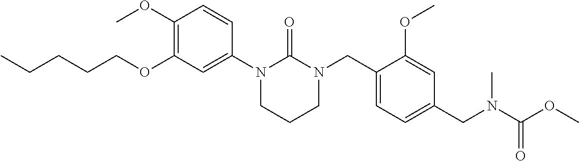 [SMILES: CCCCCOc1cc(N2CCCN(Cc3ccc(CN(C)C(=O)OC)cc3OC)C2=O)ccc1OC]

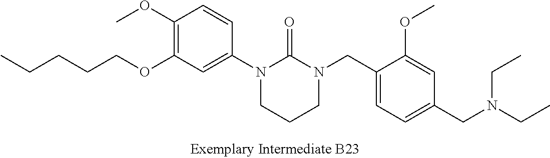 [SMILES: CCCCCOc1cc(N2CCCN(Cc3ccc(CN(CC)CC)cc3OC)C2=O)ccc1OC]

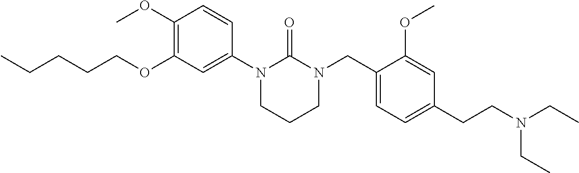 [SMILES: CCCCCOc1cc(N2CCCN(Cc3ccc(CCN(CC)CC)cc3OC)C2=O)ccc1OC]

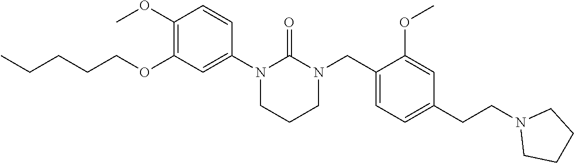 [SMILES: CCCCCOc1cc(N2CCCN(Cc3ccc(CCN4CCCC4)cc3OC)C2=O)ccc1OC]

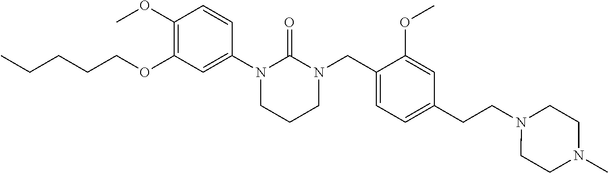 [SMILES: CCCCCOc1cc(N2CCCN(Cc3ccc(CCN4CCN(C)CC4)cc3OC)C2=O)ccc1OC]

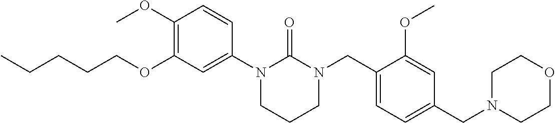 [SMILES: CCCCCOc1cc(N2CCCN(Cc3ccc(CN4CCOCC4)cc3OC)C2=O)ccc1OC]

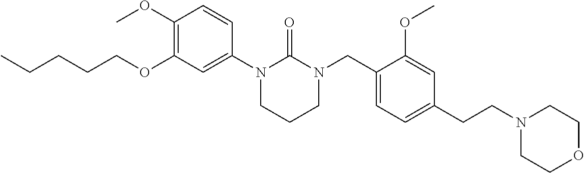 [SMILES: CCCCCOc1cc(N2CCCN(Cc3ccc(CCN4CCOCC4)cc3OC)C2=O)ccc1OC]

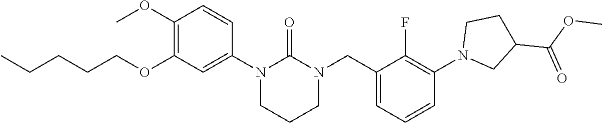 [SMILES: CCCCCOc1cc(N2CCCN(Cc3cccc(N4CCC(C(=O)OC)C4)c3F)C2=O)ccc1OC]

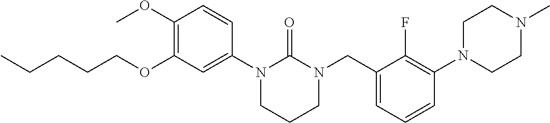 [SMILES: CCCCCOc1cc(N2CCCN(Cc3cccc(N4CCN(C)CC4)c3F)C2=O)ccc1OC]

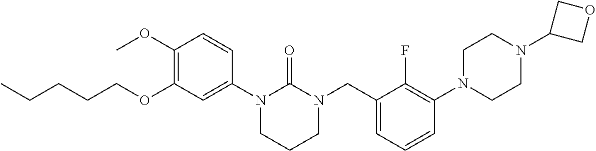 [SMILES: CCCCCOc1cc(N2CCCN(Cc3cccc(N4CCN(C5COC5)CC4)c3F)C2=O)ccc1OC]

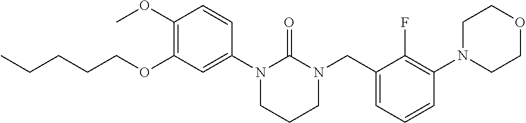 [SMILES: CCCCCOc1cc(N2CCCN(Cc3cccc(N4CCOCC4)c3F)C2=O)ccc1OC]

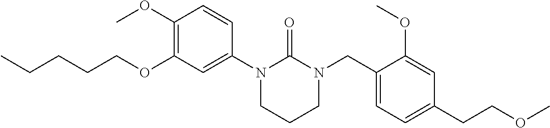 [SMILES: CCCCCOc1cc(N2CCCN(Cc3ccc(CCOC)cc3OC)C2=O)ccc1OC]

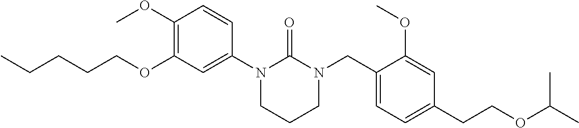 [SMILES: CCCCCOc1cc(N2CCCN(Cc3ccc(CCOC(C)C)cc3OC)C2=O)ccc1OC]

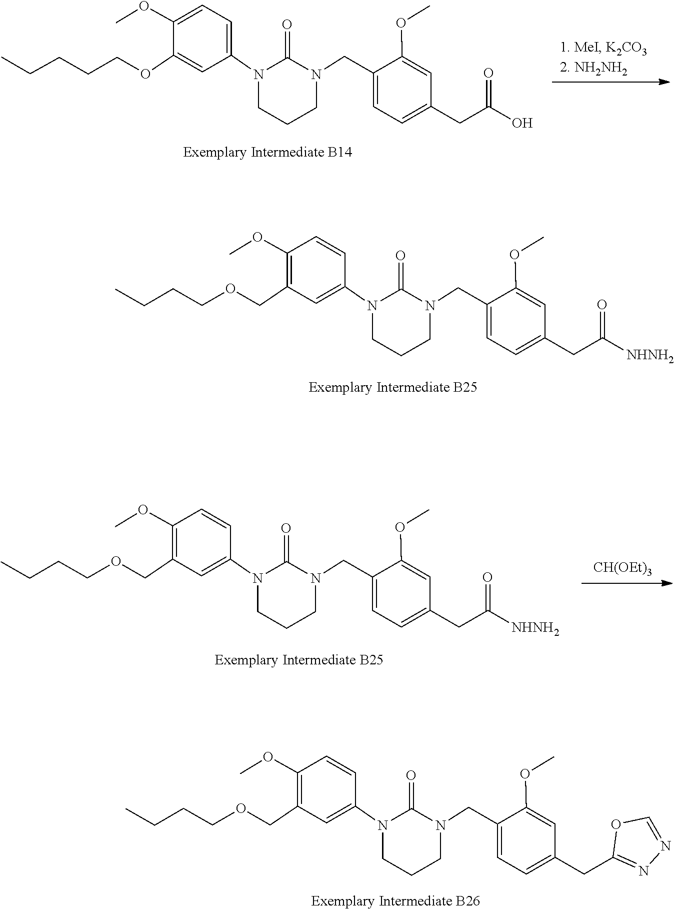 [SMILES: CCCCCOc1cc(N2CCCN(Cc3ccc(CC(=O)O)cc3OC)C2=O)ccc1OC ; CCCCOCc1cc(N2CCCN(Cc3ccc(CC(=O)NN)cc3OC)C2=O)ccc1OC ; CCCCOCc1cc(N2CCCN(Cc3ccc(CC(=O)NN)cc3OC)C2=O)ccc1OC ; CCCCOCc1cc(N2CCCN(Cc3ccc(Cc4nnco4)cc3OC)C2=O)ccc1OC ; "CH(OEt)3"]

Exemplary Intermediate B14 (2-(3-methoxy-4-((3-(4-methoxy-3-(pentyloxy)phenyl)-2-oxotetrahydropyrimidin-1(2H)-yl)methyl)phenyl)acetic acid) is combined with methyl iodide and potassium bicarbonate, and then reacted with hydrazine, affording Exemplary Intermediate B25 (2-(4-((3-(3-(butoxymethyl)-4-methoxyphenyl)-2-oxotetrahydropyrimidin-1(2H)-yl)methyl)-3-methoxyphenyl)acetohydrazide).

Exemplary Intermediate B25 is combined with triethyl orthoformate and heated under reflux for 8 h and then cooled. The resulting crystals are filtered off, washed with ether, and then dried to afford Exemplary Intermediate B26 (1-(4-((1,3,4-oxadiazol-2-yl)methyl)-2-methoxybenzyl)-3-(3-(butoxymethyl)-4-methoxyphenyl)tetrahydropyrimidin-2(1H)-one).

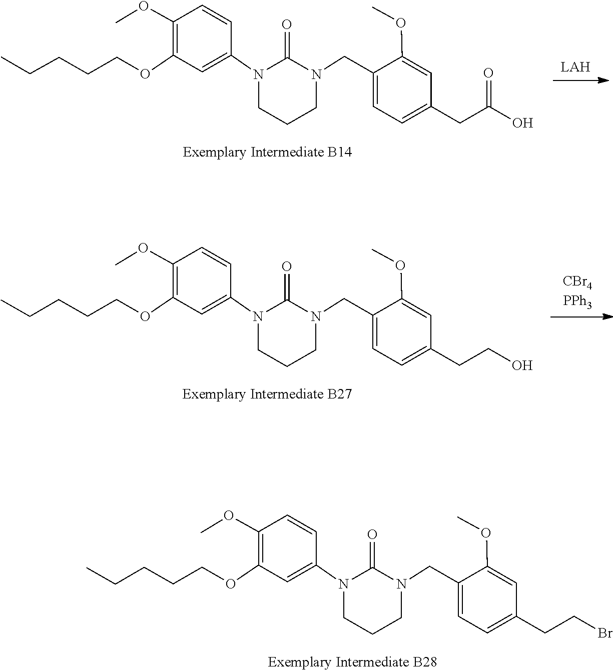 [SMILES: CCCCCOc1cc(N2CCCN(Cc3ccc(CC(=O)O)cc3OC)C2=O)ccc1OC ; CCCCCOc1cc(N2CCCN(Cc3ccc(CCO)cc3OC)C2=O)ccc1OC ; CCCCCOc1cc(N2CCCN(Cc3ccc(CCBr)cc3OC)C2=O)ccc1OC]

Exemplary Intermediate B14 (2-(3-methoxy-4-((3-(4-methoxy-3-(pentyloxy)phenyl)-2-oxotetrahydropyrimidin-1(2H)-yl)methyl)phenyl)acetic acid) is exposed to lithium aluminum hydride to afford Exemplary Intermediate B27 (1-(3-(butoxymethyl)-4-methoxyphenyl)-3-(4-(2-hydroxyethyl)-2-methoxybenzyl)tetrahydropyrimidin-2(1H)-one).

Exemplary Intermediate B27 is combined with carbon tetrabromide and triphenylphosphine to afford Exemplary Intermediate B28 (1-(4-(2-bromoethyl)-2-methoxybenzyl)-3-(3-(butoxymethyl)-4-methoxyphenyl)tetrahydropyrimidin-2(1H)-one).

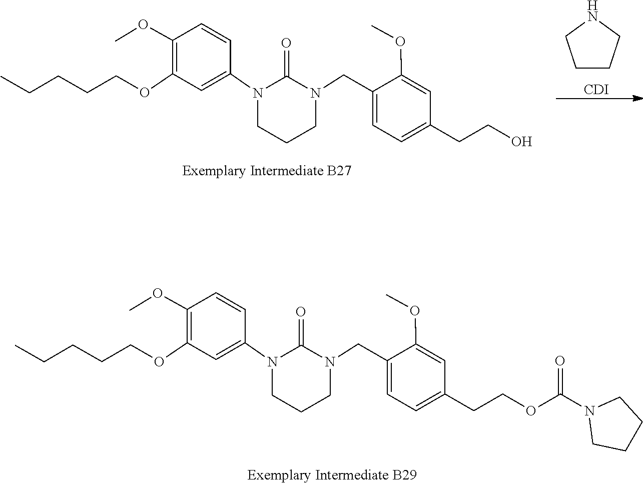

Exemplary Intermediate B27 (1-(3-(butoxymethyl)-4-methoxyphenyl)-3-(4-(2-hydroxyethyl)-2-methoxybenzyl)tetrahydropyrimidin-2(1H)-one) is reacted with pyrrolidine and 1,1-carbonyldiimidazole to afford Exemplary Intermediate B29 (4-((3-(3-(butoxymethyl)-4-methoxyphenyl)-2-oxotetrahydropyrimidin-1(2H)-yl)methyl)-3-methoxyphenethyl pyrrolidine-1-carboxylate).

The following compounds are prepared in a similar manner as Exemplary Intermediate B29, as described above.

| Alcohol | Amine |
|---|---|
| 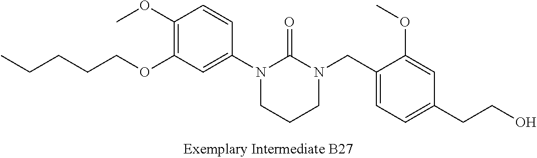 [SMILES: CCCCCOc1cc(N2CCCN(Cc3ccc(CCO)cc3OC)C2=O)ccc1OC] | 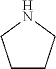 |
| 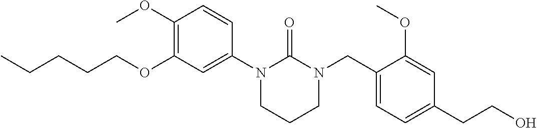 [SMILES: CCCCCOc1cc(N2CCCN(Cc3ccc(CCO)cc3OC)C2=O)ccc1OC] | MeNH₂ |
| 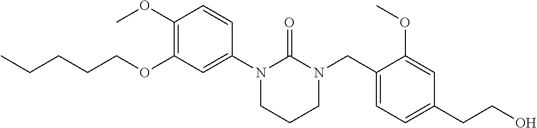 [SMILES: CCCCCOc1cc(N2CCCN(Cc3ccc(CCO)cc3OC)C2=O)ccc1OC] | Me₂NH |

Compound

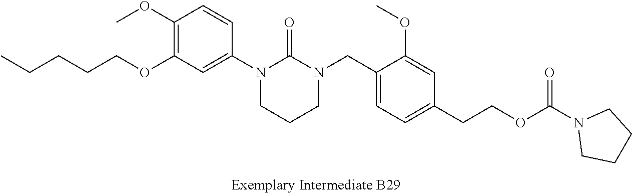 [SMILES: CCCCCOc1cc(N2CCCN(Cc3ccc(CCOC(=O)N4CCCC4)cc3OC)C2=O)ccc1OC]

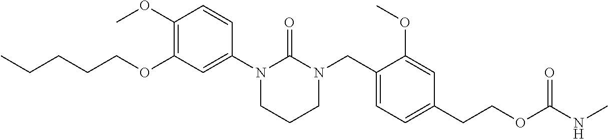 [SMILES: CCCCCOc1cc(N2CCCN(Cc3ccc(CCOC(=O)NC)cc3OC)C2=O)ccc1OC]

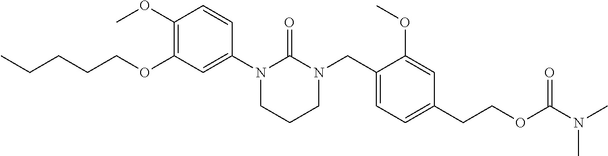 [SMILES: CCCCCOc1cc(N2CCCN(Cc3ccc(CCOC(=O)N(C)C)cc3OC)C2=O)ccc1OC]

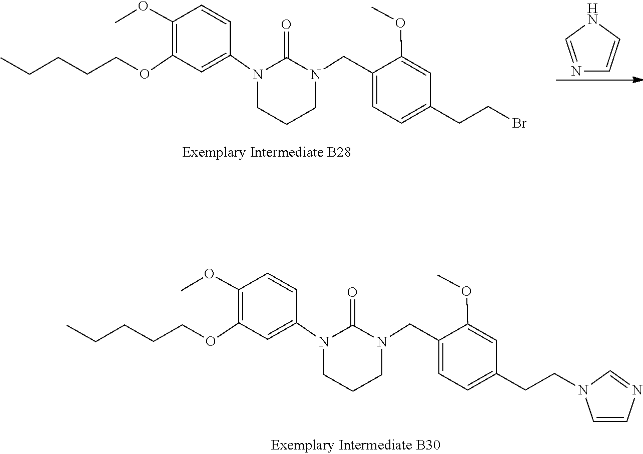 [SMILES: CCCCCOc1cc(N2CCCN(Cc3ccc(CCBr)cc3OC)C2=O)ccc1OC ; c1c[nH]cn1 ; CCCCCOc1cc(N2CCCN(Cc3ccc(CCn4ccnc4)cc3OC)C2=O)ccc1OC]

Exemplary Intermediate B28 (1-(4-(2-bromoethyl)-2-methoxybenzyl)-3-(3-(butoxymethyl)-4-methoxyphenyl)tetrahydropyrimidin-2(1H)-one) is reacted with imidazole and sodium hydride. The resulting reaction affords Exemplary Intermediate B30 (1-(4-(2-(1H-imidazol-1-yl)ethyl)-2-methoxybenzyl)-3-(3-(butoxymethyl)-4-methoxyphenyl) tetrahydropyrimidin-2(1H)-one).

The following compounds are prepared in a similar manner as Exemplary Intermediate B30 as described above.

| Bromide | Amine |
|---|---|
| 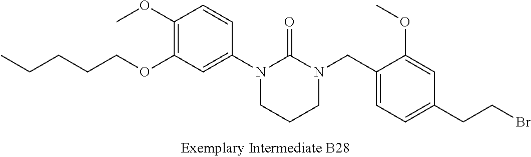 [SMILES: CCCCCOc1cc(N2CCCN(Cc3ccc(CCBr)cc3OC)C2=O)ccc1OC] | 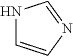 |
| 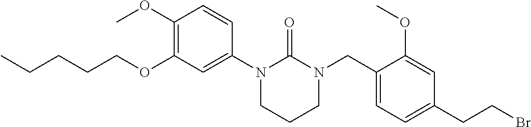 [SMILES: CCCCCOc1cc(N2CCCN(Cc3ccc(CCBr)cc3OC)C2=O)ccc1OC] | 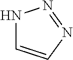 |
| 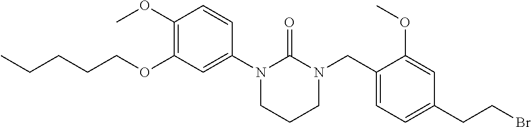 [SMILES: CCCCCOc1cc(N2CCCN(Cc3ccc(CCBr)cc3OC)C2=O)ccc1OC] | 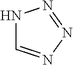 |
| 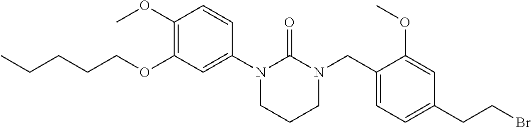 [SMILES: CCCCCOc1cc(N2CCCN(Cc3ccc(CCBr)cc3OC)C2=O)ccc1OC] | 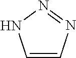 |
| 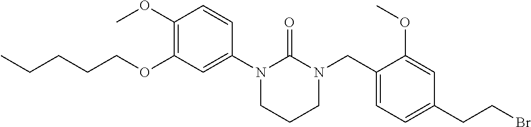 [SMILES: CCCCCOc1cc(N2CCCN(Cc3ccc(CCBr)cc3OC)C2=O)ccc1OC] | 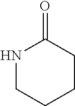 |
| 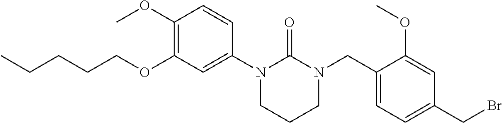 [SMILES: CCCCCOc1cc(N2CCCN(Cc3ccc(CBr)cc3OC)C2=O)ccc1OC] | 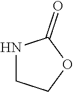 |

Compound

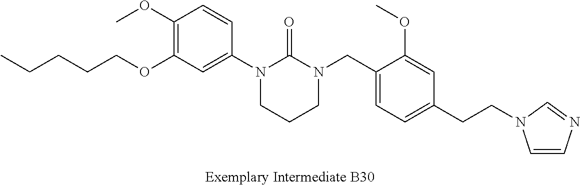 [SMILES: CCCCCOc1cc(N2CCCN(Cc3ccc(CCn4ccnc4)cc3OC)C2=O)ccc1OC]

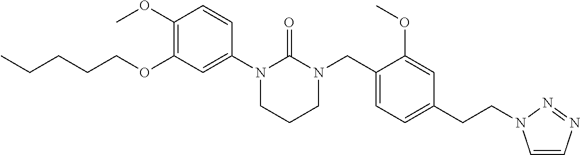 [SMILES: CCCCCOc1cc(N2CCCN(Cc3ccc(CCn4ccnn4)cc3OC)C2=O)ccc1OC]

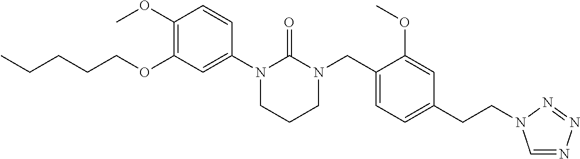 [SMILES: CCCCCOc1cc(N2CCCN(Cc3ccc(CCn4cnnn4)cc3OC)C2=O)ccc1OC]

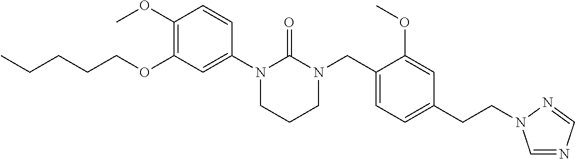 [SMILES: CCCCCOc1cc(N2CCCN(Cc3ccc(CCn4cncn4)cc3OC)C2=O)ccc1OC]

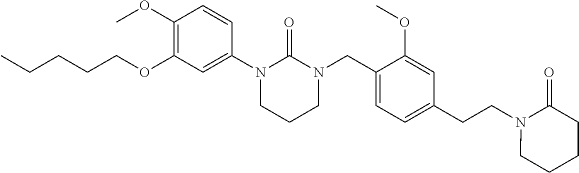 [SMILES: CCCCCOc1cc(N2CCCN(Cc3ccc(CCN4CCCCC4=O)cc3OC)C2=O)ccc1OC]

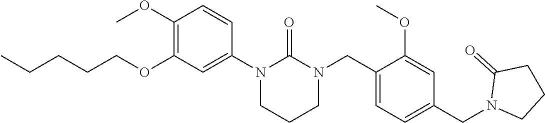 [SMILES: CCCCCOc1cc(N2CCCN(Cc3ccc(CN4CCCC4=O)cc3OC)C2=O)ccc1OC]

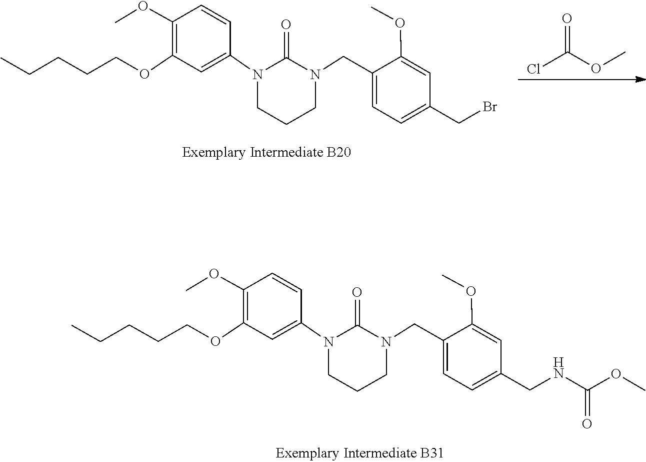

Exemplary Intermediate B20 (1-(4-(aminomethyl)-2-methoxybenzyl)-3-(4-methoxy-3-(pentyloxy)phenyl)tetrahydropyrimidin-2(1H)-one) is combined with triethylamine and methyl chloroformate to afford Exemplary Intermediate B31 (methyl (3-methoxy-4-((3-(4-methoxy-3-(pentyloxy)phenyl)-2-oxotetrahydropyrimidin-1(2H)-yl)methyl)benzyl)carbamate).

The following compounds are prepared in a similar manner as Exemplary Intermediate B31, as described above.

| Amine | Chloroformate |
|---|---|
| 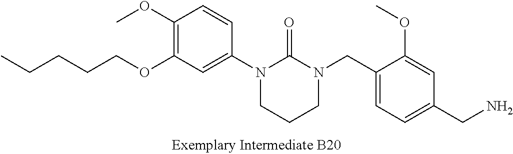 [SMILES: CCCCCOc1cc(N2CCCN(Cc3ccc(CN)cc3OC)C2=O)ccc1OC] | 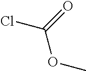 |
| 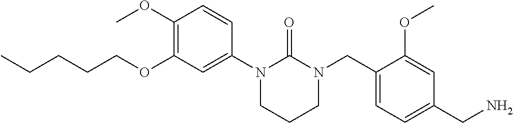 [SMILES: CCCCCOc1cc(N2CCCN(Cc3ccc(CN)cc3OC)C2=O)ccc1OC] | 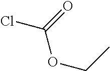 |
| 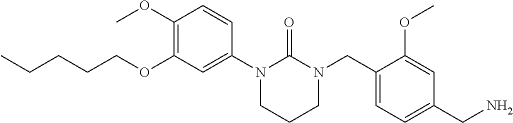 [SMILES: CCCCCOc1cc(N2CCCN(Cc3ccc(CN)cc3OC)C2=O)ccc1OC] | 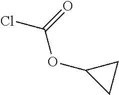 |

Compound

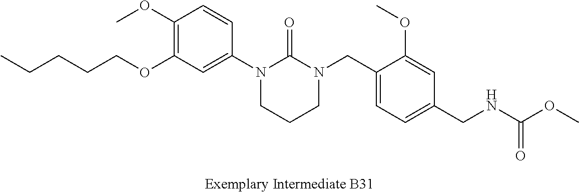 [SMILES: CCCCCOc1cc(N2CCCN(Cc3ccc(CNC(=O)OC)cc3OC)C2=O)ccc1OC]

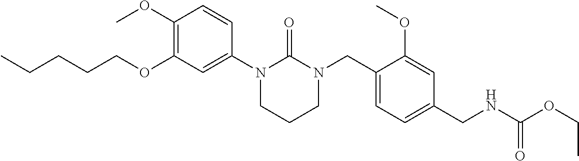 [SMILES: CCCCCOc1cc(N2CCCN(Cc3ccc(CNC(=O)OCC)cc3OC)C2=O)ccc1OC]

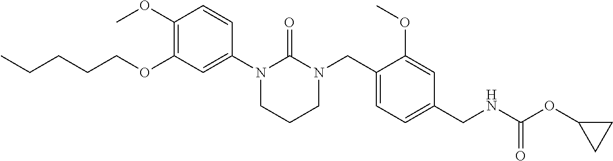 [SMILES: CCCCCOc1cc(N2CCCN(Cc3ccc(CNC(=O)OC4CC4)cc3OC)C2=O)ccc1OC]

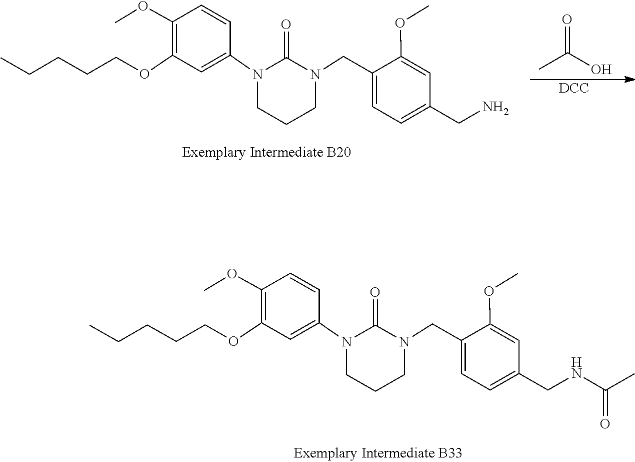

Exemplary Intermediate B20 (1-(4-(aminomethyl)-2-methoxybenzyl)-3-(4-methoxy-3-(pentyloxy)phenyl)tetrahydropyrimidin-2(1H)-one) is combined with dicyclohexylcarbodiimide (DCC) and acetic acid to afford Exemplary Intermediate B33 (N-(3-methoxy-4-((3-(4-methoxy-3-(pentyloxy)phenyl)-2-oxotetrahydropyrimidin-1(2H)-yl)methyl)benzyl)acetamide).

The following compounds are prepared in a similar manner as Exemplary Intermediate B33 as described above.

| Amine | Acid |
|---|---|
| 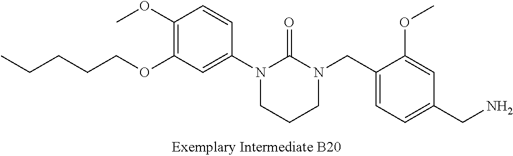 [SMILES: CCCCCOc1cc(N2CCCN(Cc3ccc(CN)cc3OC)C2=O)ccc1OC] | 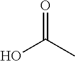 |
| 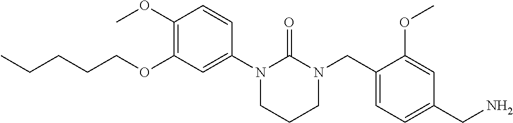 [SMILES: CCCCCOc1cc(N2CCCN(Cc3ccc(CN)cc3OC)C2=O)ccc1OC] | 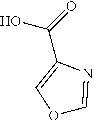 |
| 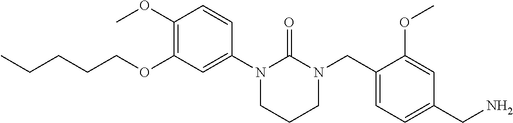 [SMILES: CCCCCOc1cc(N2CCCN(Cc3ccc(CN)cc3OC)C2=O)ccc1OC] | 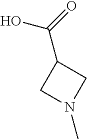 |

Compound

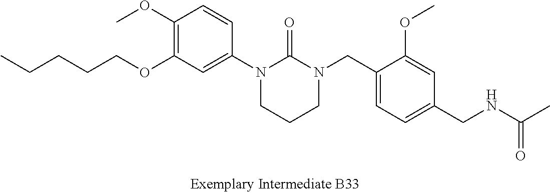 [SMILES: CCCCCOc1cc(N2CCCN(Cc3ccc(CNC(C)=O)cc3OC)C2=O)ccc1OC]

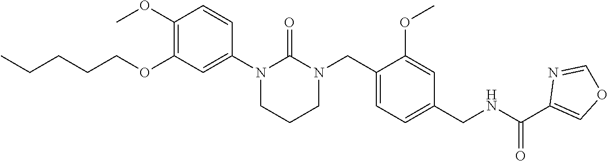 [SMILES: CCCCCOc1cc(N2CCCN(Cc3ccc(CNC(=O)c4cocn4)cc3OC)C2=O)ccc1OC]

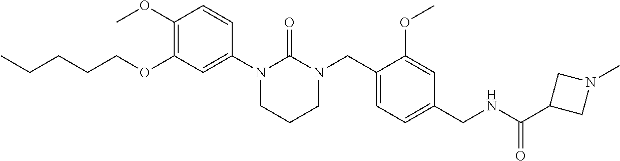 [SMILES: CCCCCOc1cc(N2CCCN(Cc3ccc(CNC(=O)C4CN(C)C4)cc3OC)C2=O)ccc1OC]

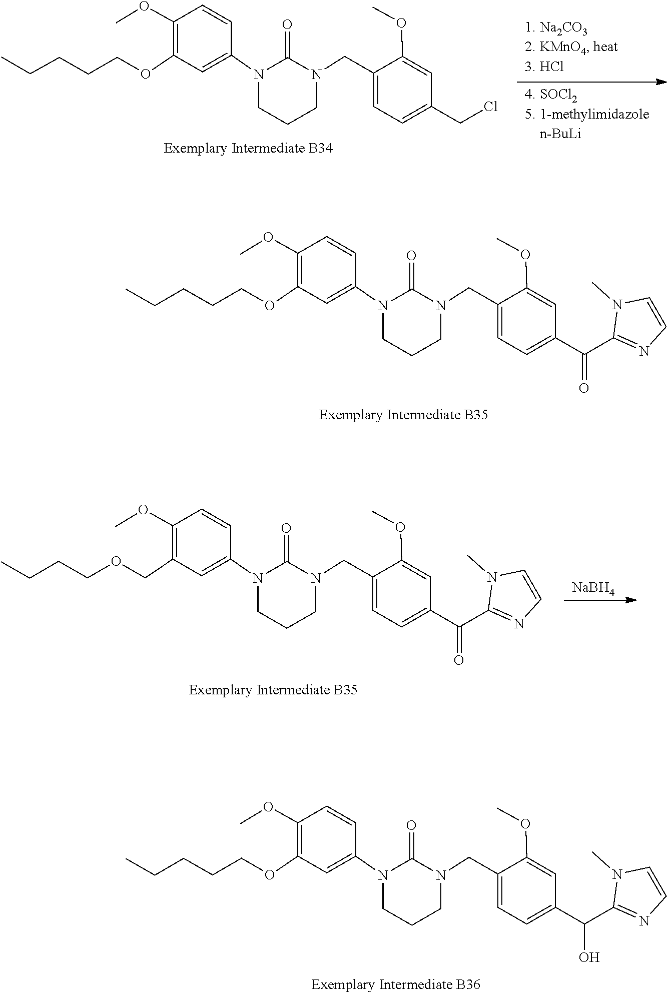

Exemplary Intermediate B34 (1-(4-(chloromethyl)-2-methoxybenzyl)-3-(4-methoxy-3-(pentyloxy)phenyl)tetrahydropyrimidin-2(1H)-one) is combined with anhydrous sodium carbonate in water, followed by the addition of potassium permanganate. The mixture is refluxed over heat for up to 2 hours. The mixture is cooled, and hydrochloric acid is added dropwise until mixture is strongly acidic, forming a benzoic acid precipitate. A 20% aqueous solution of sodium sulphite is added while stirring to dissolve any manganese dioxide precipitate. The mixture is filtered, washed with cold water, and then recrystallized from boiling water. The benzoic acid product is combined with thionyl chloride, followed by the addition of butyllithium and 1-methylimidazole, affording Exemplary Intermediate B35 (1-(4-methoxy-3-(pentyloxy)phenyl)-3-(2-methoxy-4-(1-methyl-1H-imidazole-2-carbonyl)benzyl)tetrahydropyrimidin-2(1H)-one).

Exemplary Intermediate B35 is combined with sodium borohydride to afford Exemplary Intermediate B36 (1-(4-(hydroxy(1-methyl-1H-imidazol-2-yl)methyl)-2-methoxybenzyl)-3-(4-methoxy-3-(pentyloxy)phenyl)tetrahydropyrimidin-2(1H)-one).

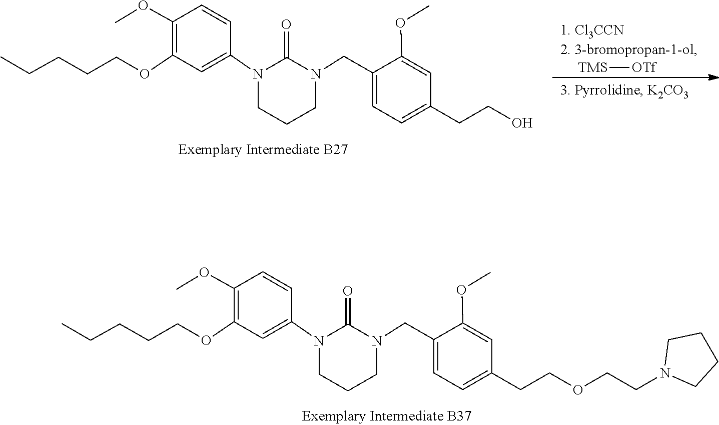 [SMILES: CCCCCOc1cc(N2CCCN(Cc3ccc(CCO)cc3OC)C2=O)ccc1OC ; C[Si](C)(C)OS(=O)(=O)C(F)(F)F ; CCCCCOc1cc(N2CCCN(Cc3ccc(CCOCCN4CCCC4)cc3OC)C2=O)ccc1OC]

Exemplary Intermediate B27 (1-(4-(2-hydroxyethyl)-2-methoxybenzyl)-3-(4-methoxy-3-(pentyloxy)phenyl)tetrahydropyrimidin-2(1H)-one) is combined with trichloroacetonitrile, followed by 3-bromopropan-1-ol with trimethylsilyl trifluoromethanesulfonate. The mixture is then combined with pyrrolidine and potassium carbonate to afford Exemplary Intermediate B37 (1-(4-methoxy-3-(pentyloxy)phenyl)-3-(2-methoxy-4-(2-(2-(pyrrolidin-1-yl)ethoxy)ethyl)benzyl)tetrahdropyrimidin-2(1H)-one).

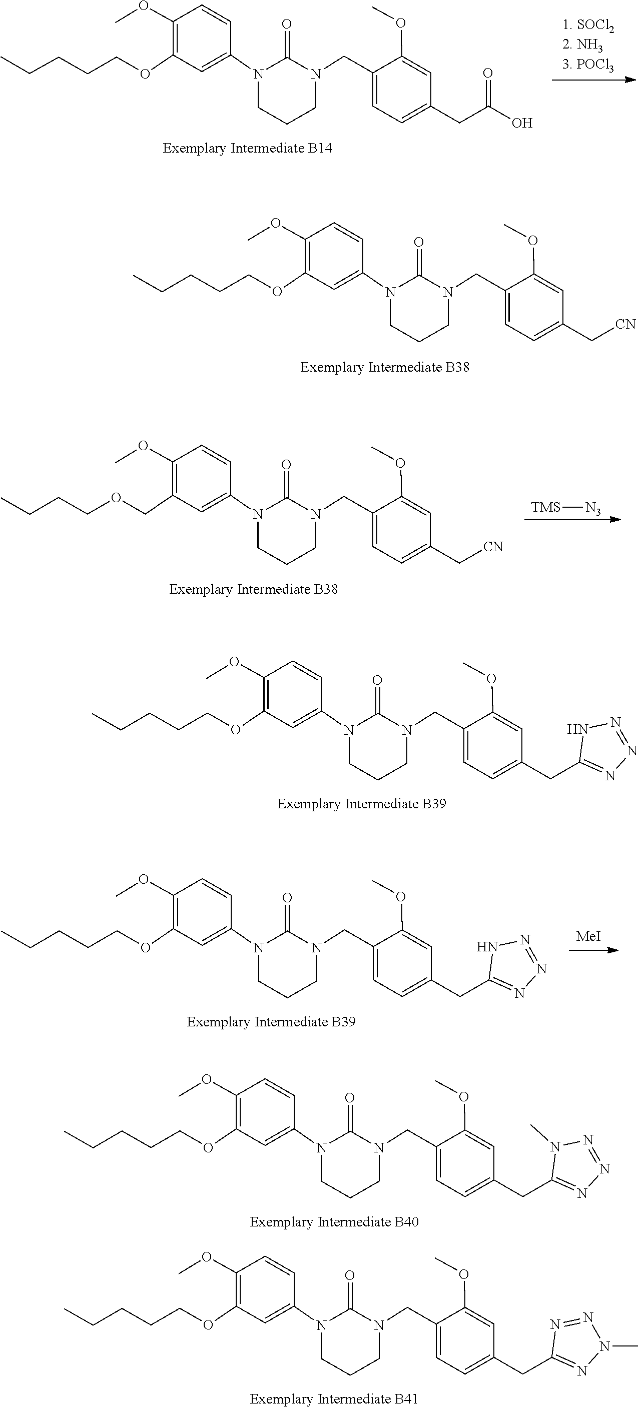

Exemplary Intermediate B14 (2-(3-methoxy-4-((3-(4-methoxy-3-(pentyloxy)phenyl)-2-oxotetrahydropyrimidin-1(2H)-yl)methyl)phenyl)acetic acid) is combined with thionyl chloride and porcelain and refluxed over a boiling water bath for about an hour (until gas evolution ceases). The mixture is cooled, and the chloride product is isolated using heat distillation. The chloride product is combined with concentrated ammonia and water, and the mixture is agitated for about 15 minutes until oily residue disappears. The amide product is collected via filtration and washed with cold water. The amide product is combined with phosphorus oxychloride to afford Exemplary Intermediate B38 (2-(3-methoxy-4-((3-(4-methoxy-3-(pentyloxy)phenyl)-2-oxotetrahydropyrimidin-1(2H)-yl)methyl)phenyl)acetonitrile).

Exemplary Intermediate B38 is reacted with trimethylsilyl azide to afford Exemplary Intermediate B39 (1-(4-((1H-tetrazol-5-yl)methyl)-2-methoxybenzyl)-3-(4-methoxy-3-(pentyloxy)phenyl)tetrahydropyrimidin-2(1H)-one).

Exemplary Intermediate B39 is reacted with methyl iodide to afford a mixture of Exemplary Intermediate B40 (1-(4-methoxy-3-(pentyloxy)phenyl)-3-(2-methoxy-4-((1-methyl-1H-tetrazol-5-yl)methyl)benzyl)tetrahydropyrimidin-2(1H)-one) and Exemplary Intermediate B41 (1-(4-methoxy-3-(pentyloxy)phenyl)-3-(2-methoxy-4-((2-methyl-2H-tetrazol-5-yl)methyl)benzyl)tetrahydropyrimidin-2(1H)-one).

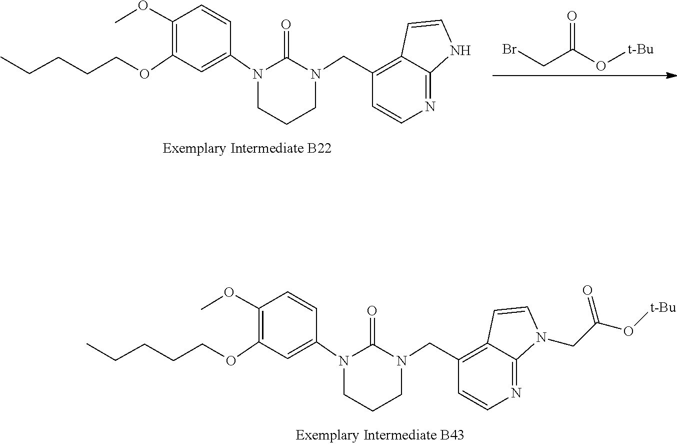 [SMILES: CCCCCOc1cc(N2CCCN(Cc3ccnc4[nH]ccc34)C2=O)ccc1OC ; CC(C)(C)OC(=O)CBr ; CCCCCOc1cc(N2CCCN(Cc3ccnc4c3ccn4CC(=O)OC(C)(C)C)C2=O)ccc1OC]

To a solution of Exemplary Intermediate B22 (1-((1H-pyrrolo[2,3-b]pyridin-4-yl)methyl)-3-(4-methoxy-3-(pentyloxy)phenyl)tetrahydropyrimidin-2(1H)-one) in N,N-dimethylformamide is added sodium hydride in portions at room temperature. The mixture is stirred at room temperature for 10 min then cooled to 0° C., and a solution of tert-butyl bromoacetate in N,N-dimethylformamide is added dropwise. The reaction is slowly warmed to room temperature and stirred for 3.5 h. Water is slowly added to the reaction, and the resulting mixture is extracted twice with ethyl acetate. The combined organic layers are washed with brine, dried over sodium sulfate, and concentrated in vacuo. The residue is purified by column chromatography over silica gel (dichloromethane/methanol: 100/0 to 97/3) to afford Exemplary Intermediate B43 (tert-butyl 2-(4-((3-(4-methoxy-3-(pentyloxy)phenyl)-2-oxotetrahydropyrimidin-1(2H)-yl)methyl)-1H-pyrrolo[2,3-b]pyridin-1-yl)acetate).

The following compounds are prepared in a similar manner as Exemplary Intermediate B43, as described above.

| Heterocycle | Bromide |
|---|---|
| 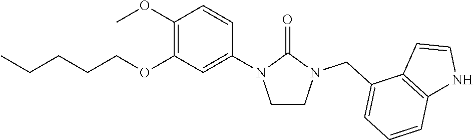 [SMILES: CCCCCOc1cc(N2CCN(Cc3cccc4[nH]ccc34)C2=O)ccc1OC] | 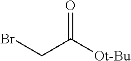 |
| 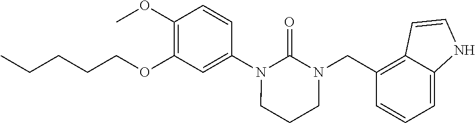 [SMILES: CCCCCOc1cc(N2CCCN(Cc3cccc4[nH]ccc34)C2=O)ccc1OC] | 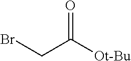 |
| 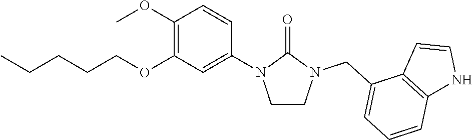 [SMILES: CCCCCOc1cc(N2CCN(Cc3cccc4[nH]ccc34)C2=O)ccc1OC] | 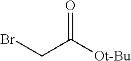 |
| 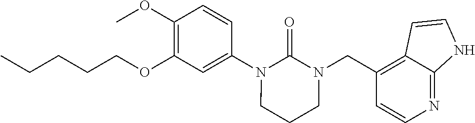 [SMILES: CCCCCOc1cc(N2CCCN(Cc3ccnc4[nH]ccc34)C2=O)ccc1OC] | 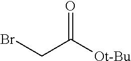 |
| 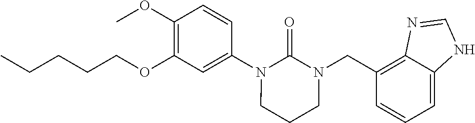 [SMILES: CCCCCOc1cc(N2CCCN(Cc3cccc4[nH]cnc34)C2=O)ccc1OC] | 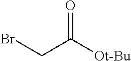 |
| 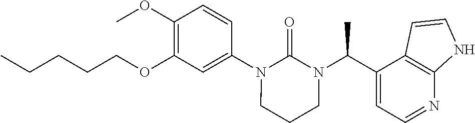 [SMILES: CCCCCOc1cc(N2CCCN([C@@H](C)c3ccnc4[nH]ccc34)C2=O)ccc1OC] | 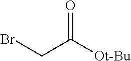 |
| 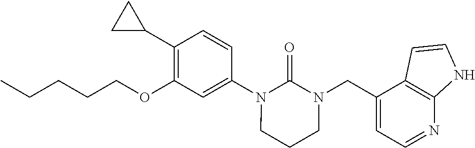 [SMILES: CCCCCOc1cc(N2CCCN(Cc3ccnc4[nH]ccc34)C2=O)ccc1C1CC1] | 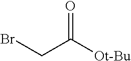 |
| 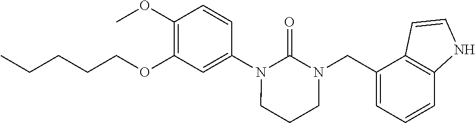 [SMILES: CCCCCOc1cc(N2CCCN(Cc3cccc4[nH]ccc34)C2=O)ccc1OC] | 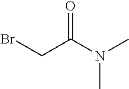 |
| 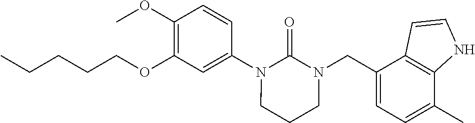 [SMILES: CCCCCOc1cc(N2CCCN(Cc3ccc(C)c4[nH]ccc34)C2=O)ccc1OC] | 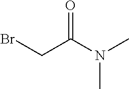 |
| 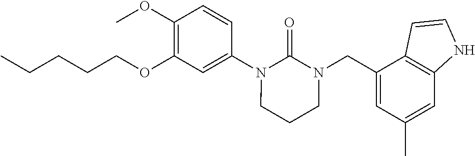 [SMILES: CCCCCOc1cc(N2CCCN(Cc3cc(C)cc4[nH]ccc34)C2=O)ccc1OC] | 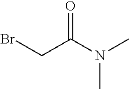 |
| 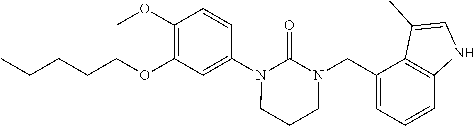 [SMILES: CCCCCOc1cc(N2CCCN(Cc3cccc4[nH]cc(C)c34)C2=O)ccc1OC] | 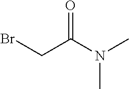 |
| 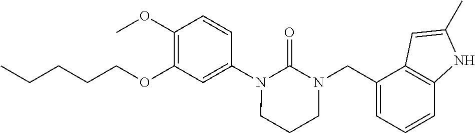 [SMILES: CCCCCOc1cc(N2CCCN(Cc3cccc4[nH]c(C)cc34)C2=O)ccc1OC] | 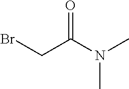 |
| 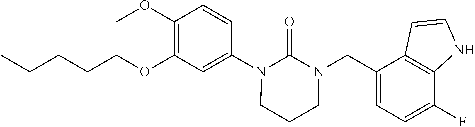 [SMILES: CCCCCOc1cc(N2CCCN(Cc3ccc(F)c4[nH]ccc34)C2=O)ccc1OC] | 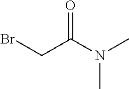 |
| 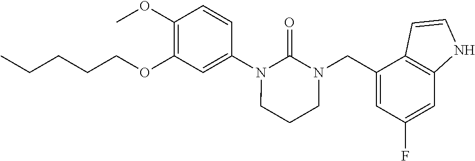 [SMILES: CCCCCOc1cc(N2CCCN(Cc3cc(F)cc4[nH]ccc34)C2=O)ccc1OC] | 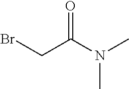 |
| 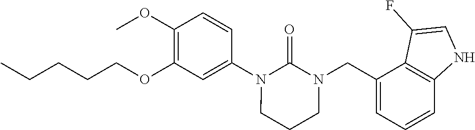 [SMILES: CCCCCOc1cc(N2CCCN(Cc3cccc4[nH]cc(F)c34)C2=O)ccc1OC] | 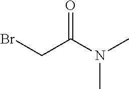 |
| 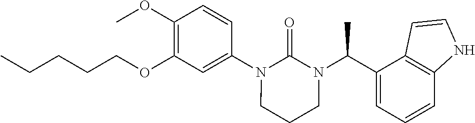 [SMILES: CCCCCOc1cc(N2CCCN([C@@H](C)c3cccc4[nH]ccc34)C2=O)ccc1OC] | 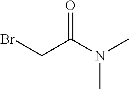 |
| 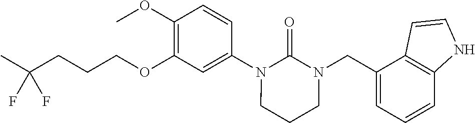 [SMILES: COc1ccc(N2CCCN(Cc3cccc4[nH]ccc34)C2=O)cc1OCCCC(C)(F)F] | 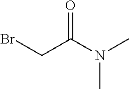 |
| 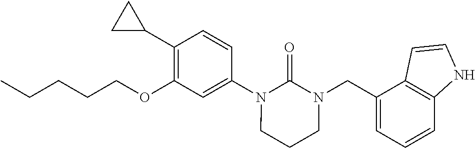 [SMILES: CCCCCOc1cc(N2CCCN(Cc3cccc4[nH]ccc34)C2=O)ccc1C1CC1] | 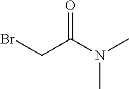 |
| 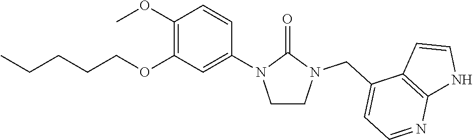 [SMILES: CCCCCOc1cc(N2CCN(Cc3ccnc4[nH]ccc34)C2=O)ccc1OC] | 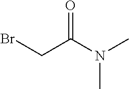 |
| 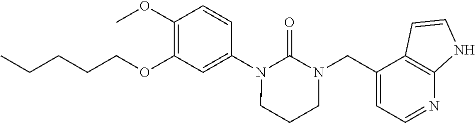 [SMILES: CCCCCOc1cc(N2CCCN(Cc3ccnc4[nH]ccc34)C2=O)ccc1OC] | 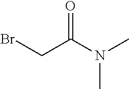 |
| 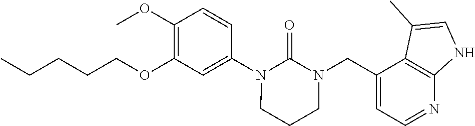 [SMILES: CCCCCOc1cc(N2CCCN(Cc3ccnc4[nH]cc(C)c34)C2=O)ccc1OC] | 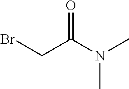 |
| 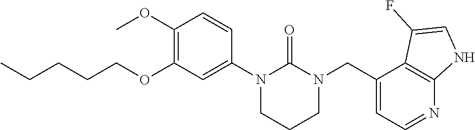 [SMILES: CCCCCOc1cc(N2CCCN(Cc3ccnc4[nH]cc(F)c34)C2=O)ccc1OC] | 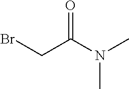 |
| 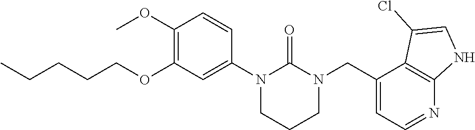 [SMILES: CCCCCOc1cc(N2CCCN(Cc3ccnc4[nH]cc(Cl)c34)C2=O)ccc1OC] | 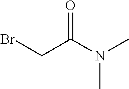 |
| 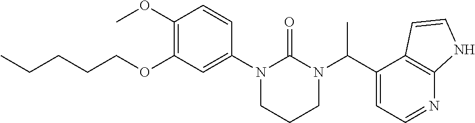 [SMILES: CCCCCOc1cc(N2CCCN(C(C)c3ccnc4[nH]ccc34)C2=O)ccc1OC] | 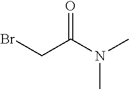 |
| 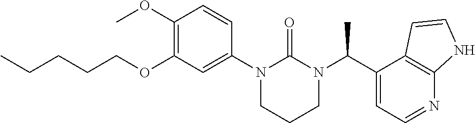 [SMILES: CCCCCOc1cc(N2CCCN([C@@H](C)c3ccnc4[nH]ccc34)C2=O)ccc1OC] | 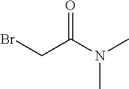 |
| 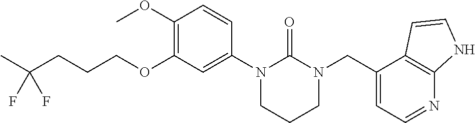 [SMILES: COc1ccc(N2CCCN(Cc3ccnc4[nH]ccc34)C2=O)cc1OCCCC(C)(F)F] | 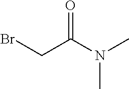 |
| 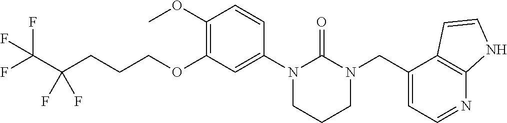 [SMILES: COc1ccc(N2CCCN(Cc3ccnc4[nH]ccc34)C2=O)cc1OCCCC(F)(F)C(F)(F)F] | 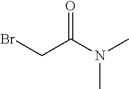 |
| 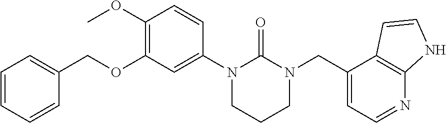 [SMILES: COc1ccc(N2CCCN(Cc3ccnc4[nH]ccc34)C2=O)cc1OCc1ccccc1] | 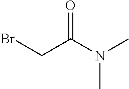 |
| 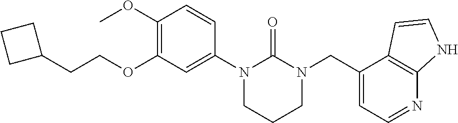 [SMILES: COc1ccc(N2CCCN(Cc3ccnc4[nH]ccc34)C2=O)cc1OCCC1CCC1] | 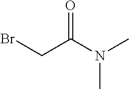 |
| 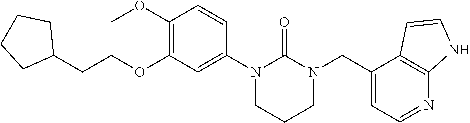 [SMILES: COc1ccc(N2CCCN(Cc3ccnc4[nH]ccc34)C2=O)cc1OCCC1CCCC1] | 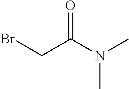 |
| 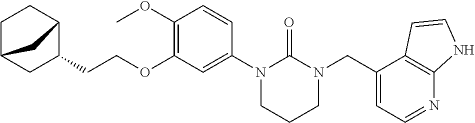 [SMILES: COc1ccc(N2CCCN(Cc3ccnc4[nH]ccc34)C2=O)cc1OCC[C@@H]1C[C@@H]2CC[C@H]1C2] | 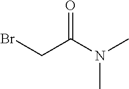 |
| 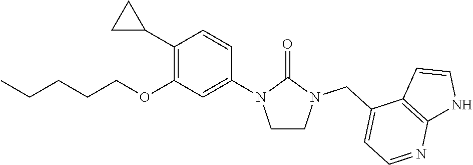 [SMILES: CCCCCOc1cc(N2CCN(Cc3ccnc4[nH]ccc34)C2=O)ccc1C1CC1] | 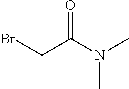 |
| 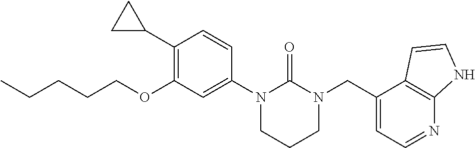 [SMILES: CCCCCOc1cc(N2CCCN(Cc3ccnc4[nH]ccc34)C2=O)ccc1C1CC1] | 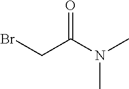 |
| 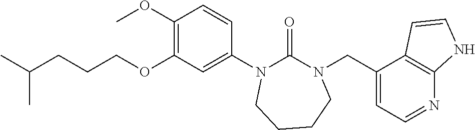 [SMILES: COc1ccc(N2CCCCN(Cc3ccnc4[nH]ccc34)C2=O)cc1OCCCC(C)C] | 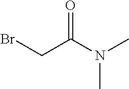 |
| 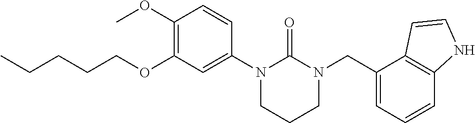 [SMILES: CCCCCOc1cc(N2CCCN(Cc3cccc4[nH]ccc34)C2=O)ccc1OC] | 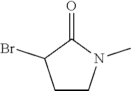 |
| 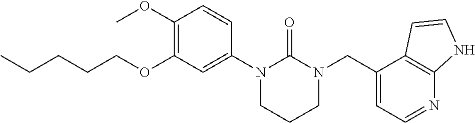 [SMILES: CCCCCOc1cc(N2CCCN(Cc3ccnc4[nH]ccc34)C2=O)ccc1OC] | 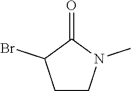 |
| 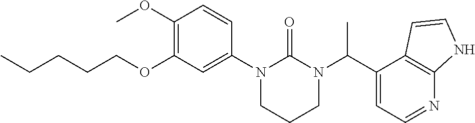 [SMILES: CCCCCOc1cc(N2CCCN(C(C)c3ccnc4[nH]ccc34)C2=O)ccc1OC] | 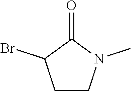 |
| 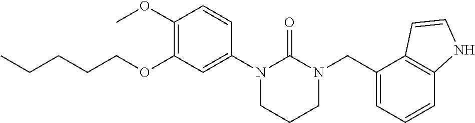 [SMILES: CCCCCOc1cc(N2CCCN(Cc3cccc4[nH]ccc34)C2=O)ccc1OC] | 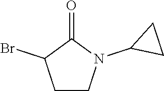 |
| 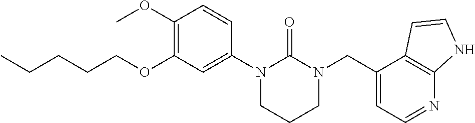 [SMILES: CCCCCOc1cc(N2CCCN(Cc3ccnc4[nH]ccc34)C2=O)ccc1OC] | 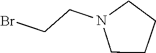 |
| 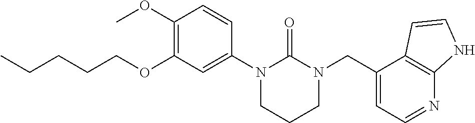 [SMILES: CCCCCOc1cc(N2CCCN(Cc3ccnc4[nH]ccc34)C2=O)ccc1OC] | 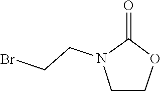 |
| 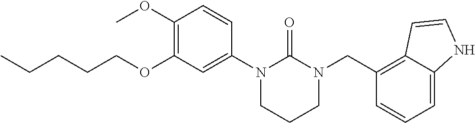 [SMILES: CCCCCOc1cc(N2CCCN(Cc3cccc4[nH]ccc34)C2=O)ccc1OC] | 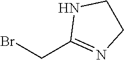 |
| 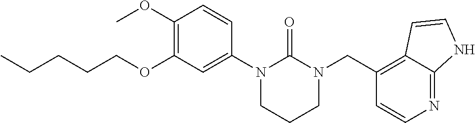 [SMILES: CCCCCOc1cc(N2CCCN(Cc3ccnc4[nH]ccc34)C2=O)ccc1OC] | 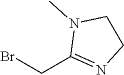 |
| 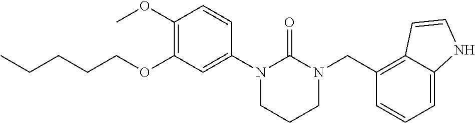 [SMILES: CCCCCOc1cc(N2CCCN(Cc3cccc4[nH]ccc34)C2=O)ccc1OC] | 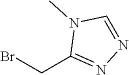 |
| 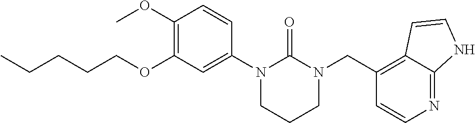 [SMILES: CCCCCOc1cc(N2CCCN(Cc3ccnc4[nH]ccc34)C2=O)ccc1OC] | 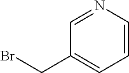 |
| 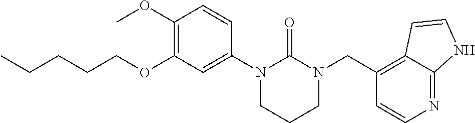 [SMILES: CCCCCOc1cc(N2CCCN(Cc3ccnc4[nH]ccc34)C2=O)ccc1OC] | 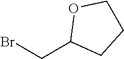 |
| 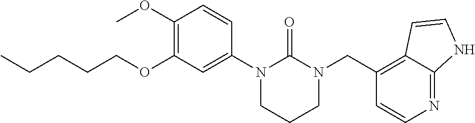 [SMILES: CCCCCOc1cc(N2CCCN(Cc3ccnc4[nH]ccc34)C2=O)ccc1OC] | 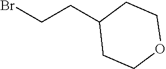 |

Compound

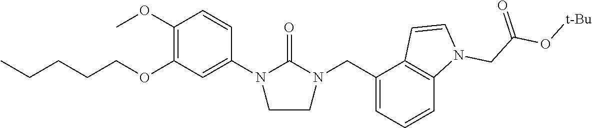 [SMILES: CCCCCOc1cc(N2CCN(Cc3cccc4c3ccn4CC(=O)OC(C)(C)C)C2=O)ccc1OC]

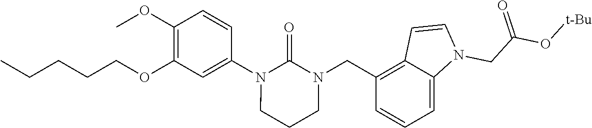 [SMILES: CCCCCOc1cc(N2CCCN(Cc3cccc4c3ccn4CC(=O)OC(C)(C)C)C2=O)ccc1OC]

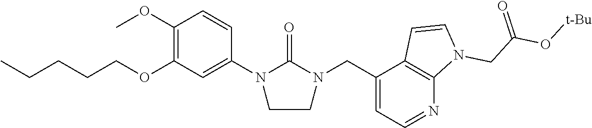 [SMILES: CCCCCOc1cc(N2CCN(Cc3ccnc4c3ccn4CC(=O)OC(C)(C)C)C2=O)ccc1OC]

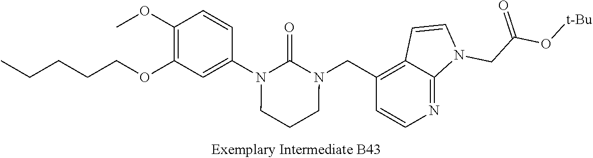 [SMILES: CCCCCOc1cc(N2CCCN(Cc3ccnc4c3ccn4CC(=O)OC(C)(C)C)C2=O)ccc1OC]

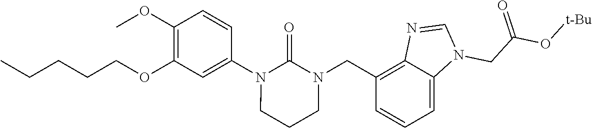 [SMILES: CCCCCOc1cc(N2CCCN(Cc3cccc4c3ncn4CC(=O)OC(C)(C)C)C2=O)ccc1OC]

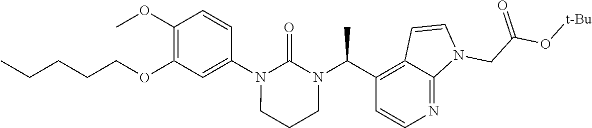 [SMILES: CCCCCOc1cc(N2CCCN([C@@H](C)c3ccnc4c3ccn4CC(=O)OC(C)(C)C)C2=O)ccc1OC]

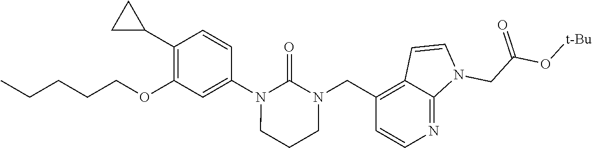 [SMILES: CCCCCOc1cc(N2CCCN(Cc3ccnc4c3ccn4CC(=O)OC(C)(C)C)C2=O)ccc1C1CC1]

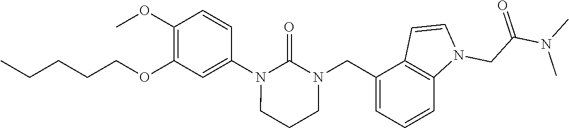 [SMILES: CCCCCOc1cc(N2CCCN(Cc3cccc4c3ccn4CC(=O)N(C)C)C2=O)ccc1OC]

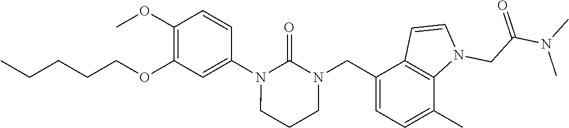 [SMILES: CCCCCOc1cc(N2CCCN(Cc3ccc(C)c4c3ccn4CC(=O)N(C)C)C2=O)ccc1OC]

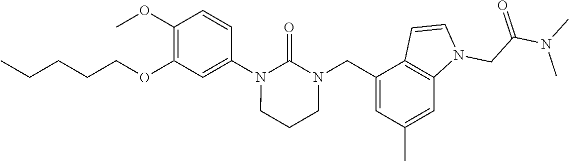 [SMILES: CCCCCOc1cc(N2CCCN(Cc3cc(C)cc4c3ccn4CC(=O)N(C)C)C2=O)ccc1OC]

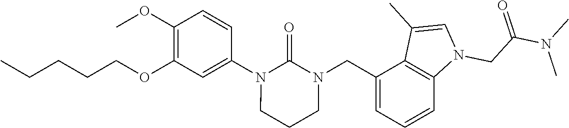 [SMILES: CCCCCOc1cc(N2CCCN(Cc3cccc4c3c(C)cn4CC(=O)N(C)C)C2=O)ccc1OC]

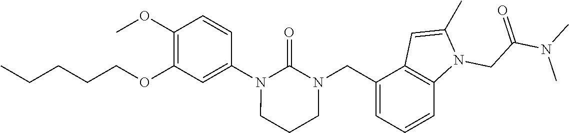 [SMILES: CCCCCOc1cc(N2CCCN(Cc3cccc4c3cc(C)n4CC(=O)N(C)C)C2=O)ccc1OC]

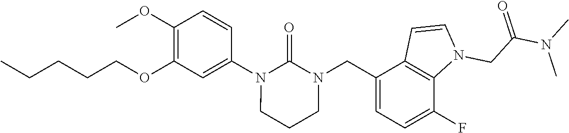 [SMILES: CCCCCOc1cc(N2CCCN(Cc3ccc(F)c4c3ccn4CC(=O)N(C)C)C2=O)ccc1OC]

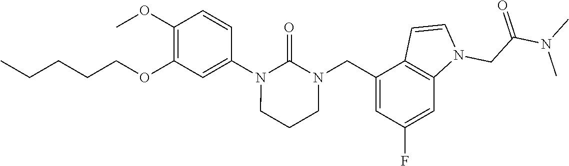 [SMILES: CCCCCOc1cc(N2CCCN(Cc3cc(F)cc4c3ccn4CC(=O)N(C)C)C2=O)ccc1OC]

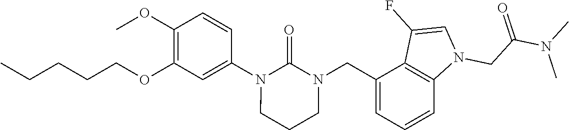 [SMILES: CCCCCOc1cc(N2CCCN(Cc3cccc4c3c(F)cn4CC(=O)N(C)C)C2=O)ccc1OC]

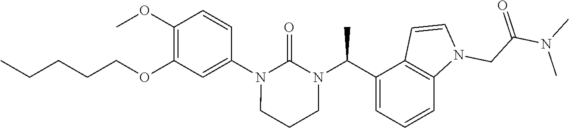 [SMILES: CCCCCOc1cc(N2CCCN([C@@H](C)c3cccc4c3ccn4CC(=O)N(C)C)C2=O)ccc1OC]

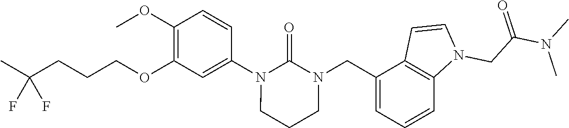 [SMILES: COc1ccc(N2CCCN(Cc3cccc4c3ccn4CC(=O)N(C)C)C2=O)cc1OCCCC(C)(F)F]

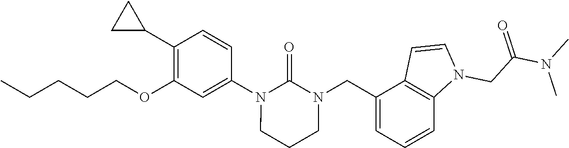 [SMILES: CCCCCOc1cc(N2CCCN(Cc3cccc4c3ccn4CC(=O)N(C)C)C2=O)ccc1C1CC1]

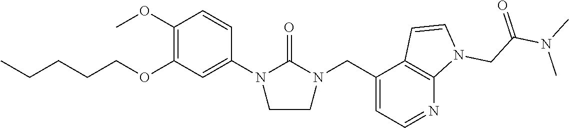 [SMILES: CCCCCOc1cc(N2CCN(Cc3ccnc4c3ccn4CC(=O)N(C)C)C2=O)ccc1OC]

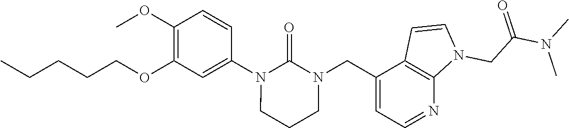 [SMILES: CCCCCOc1cc(N2CCCN(Cc3ccnc4c3ccn4CC(=O)N(C)C)C2=O)ccc1OC]

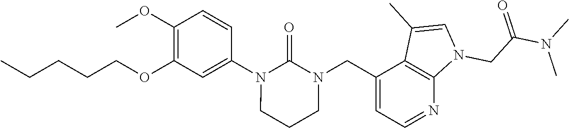 [SMILES: CCCCCOc1cc(N2CCCN(Cc3ccnc4c3c(C)cn4CC(=O)N(C)C)C2=O)ccc1OC]

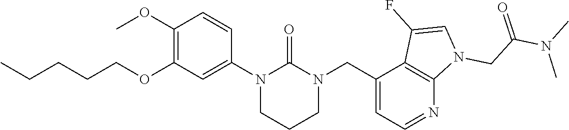 [SMILES: CCCCCOc1cc(N2CCCN(Cc3ccnc4c3c(F)cn4CC(=O)N(C)C)C2=O)ccc1OC]

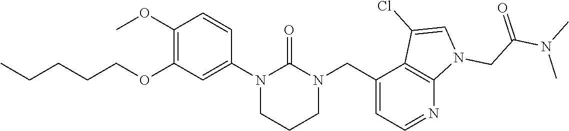 [SMILES: CCCCCOc1cc(N2CCCN(Cc3ccnc4c3c(Cl)cn4CC(=O)N(C)C)C2=O)ccc1OC]

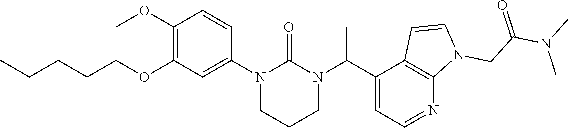 [SMILES: CCCCCOc1cc(N2CCCN(C(C)c3ccnc4c3ccn4CC(=O)N(C)C)C2=O)ccc1OC]

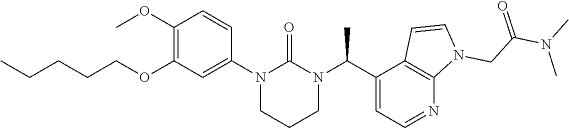 [SMILES: CCCCCOc1cc(N2CCCN([C@@H](C)c3ccnc4c3ccn4CC(=O)N(C)C)C2=O)ccc1OC]

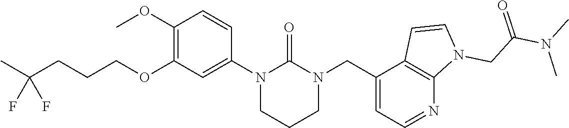 [SMILES: COc1ccc(N2CCCN(Cc3ccnc4c3ccn4CC(=O)N(C)C)C2=O)cc1OCCCC(C)(F)F]

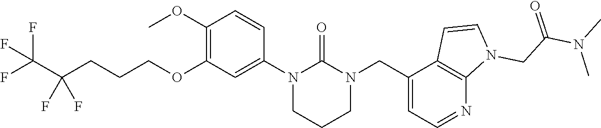 [SMILES: COc1ccc(N2CCCN(Cc3ccnc4c3ccn4CC(=O)N(C)C)C2=O)cc1OCCCC(F)(F)C(F)(F)F]

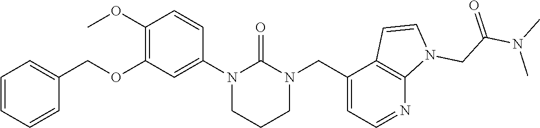 [SMILES: COc1ccc(N2CCCN(Cc3ccnc4c3ccn4CC(=O)N(C)C)C2=O)cc1OCc1ccccc1]

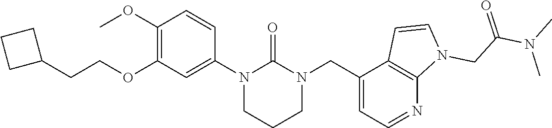 [SMILES: COc1ccc(N2CCCN(Cc3ccnc4c3ccn4CC(=O)N(C)C)C2=O)cc1OCCC1CCC1]

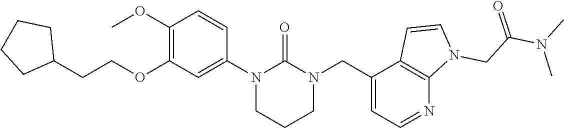 [SMILES: COc1ccc(N2CCCN(Cc3ccnc4c3ccn4CC(=O)N(C)C)C2=O)cc1OCCC1CCCC1]

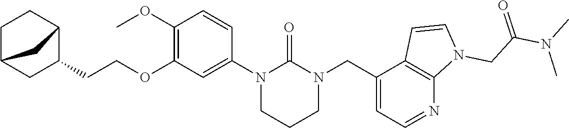 [SMILES: COc1ccc(N2CCCN(Cc3ccnc4c3ccn4CC(=O)N(C)C)C2=O)cc1OCC[C@@H]1C[C@@H]2CC[C@H]1C2]

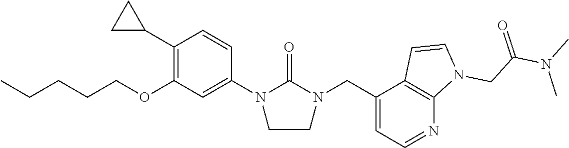 [SMILES: CCCCCOc1cc(N2CCN(Cc3ccnc4c3ccn4CC(=O)N(C)C)C2=O)ccc1C1CC1]

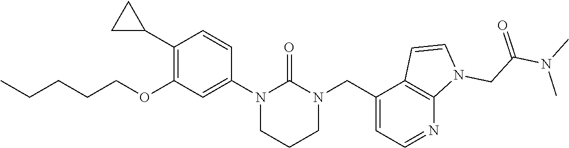 [SMILES: CCCCCOc1cc(N2CCCN(Cc3ccnc4c3ccn4CC(=O)N(C)C)C2=O)ccc1C1CC1]

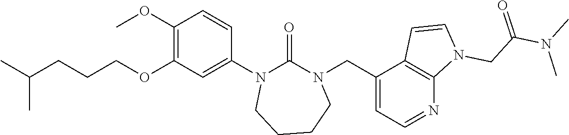 [SMILES: COc1ccc(N2CCCCN(Cc3ccnc4c3ccn4CC(=O)N(C)C)C2=O)cc1OCCCC(C)C]

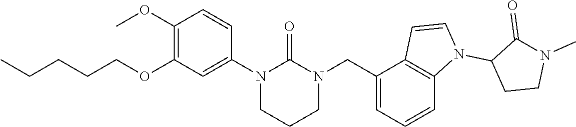 [SMILES: CCCCCOc1cc(N2CCCN(Cc3cccc4c3ccn4C3CCN(C)C3=O)C2=O)ccc1OC]

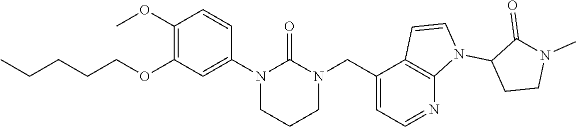 [SMILES: CCCCCOc1cc(N2CCCN(Cc3ccnc4c3ccn4C3CCN(C)C3=O)C2=O)ccc1OC]

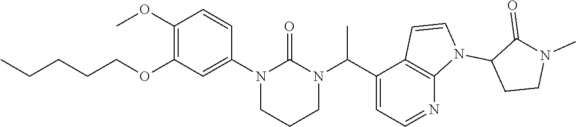 [SMILES: CCCCCOc1cc(N2CCCN(C(C)c3ccnc4c3ccn4C3CCN(C)C3=O)C2=O)ccc1OC]

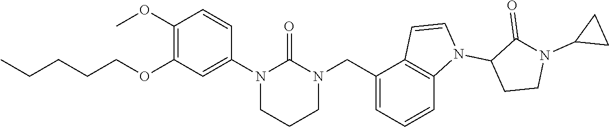 [SMILES: CCCCCOc1cc(N2CCCN(Cc3cccc4c3ccn4C3CCN(C4CC4)C3=O)C2=O)ccc1OC]

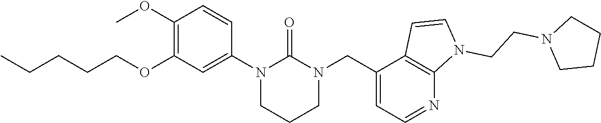 [SMILES: CCCCCOc1cc(N2CCCN(Cc3ccnc4c3ccn4CCN3CCCC3)C2=O)ccc1OC]

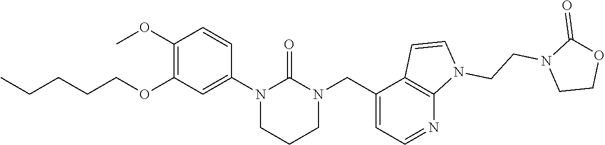 [SMILES: CCCCCOc1cc(N2CCCN(Cc3ccnc4c3ccn4CCN3CCOC3=O)C2=O)ccc1OC]

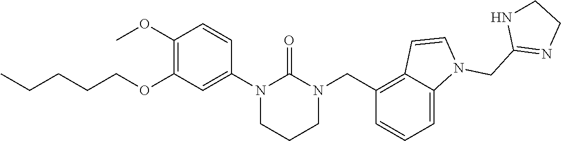 [SMILES: CCCCCOc1cc(N2CCCN(Cc3cccc4c3ccn4CC3=NCCN3)C2=O)ccc1OC]

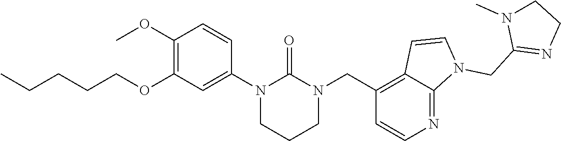 [SMILES: CCCCCOc1cc(N2CCCN(Cc3ccnc4c3ccn4CC3=NCCN3C)C2=O)ccc1OC]

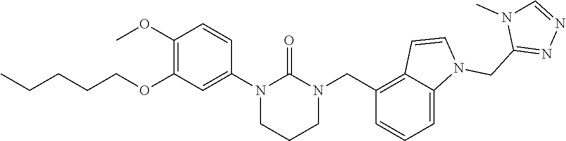 [SMILES: CCCCCOc1cc(N2CCCN(Cc3cccc4c3ccn4Cc3nncn3C)C2=O)ccc1OC]

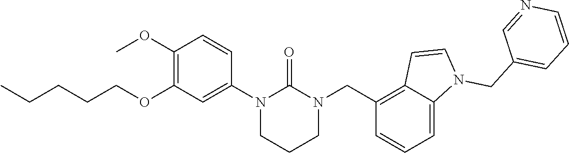 [SMILES: CCCCCOc1cc(N2CCCN(Cc3cccc4c3ccn4Cc3cccnc3)C2=O)ccc1OC]

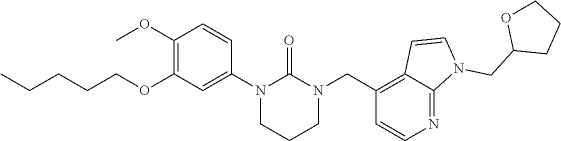 [SMILES: CCCCCOc1cc(N2CCCN(Cc3ccnc4c3ccn4CC3CCCO3)C2=O)ccc1OC]

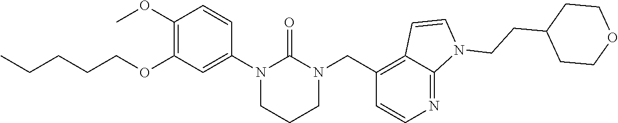 [SMILES: CCCCCOc1cc(N2CCCN(Cc3ccnc4c3ccn4CCC3CCOCC3)C2=O)ccc1OC]

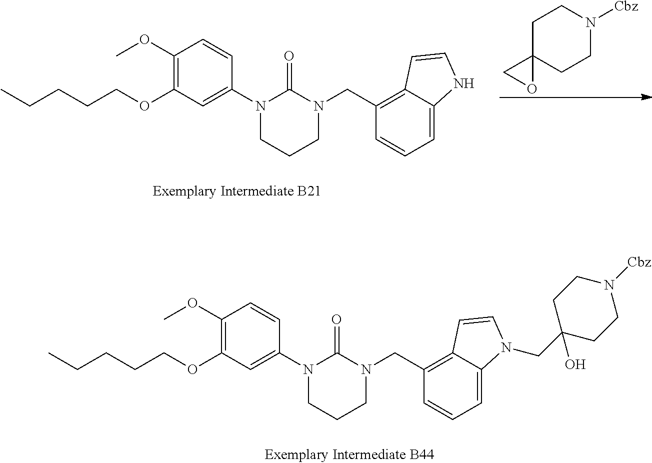

To a solution of Exemplary Intermediate B21 (1-((1H-indol-4-yl)methyl)-3-(4-methoxy-3-(pentyloxy)phenyl)tetrahydropyrimidin-2(1H)-one) in N,N-dimethylformamide is added sodium hydride in portions at room temperature. The mixture is stirred at room temperature for 10 min then cooled to 0° C., and a solution of benzyl 1-oxa-6-azaspiro[2.5]octane-6-carboxylate in N,N-dimethylformamide is added dropwise. The reaction is slowly warmed to room temperature and stirred for 3.5 h. Water is slowly added to the reaction, and the resulting mixture is extracted twice with ethyl acetate. The combined organic layers are washed with brine, dried over sodium sulfate, and concentrated in vacuo. The residue is purified by column chromatography over silica gel (dichloromethane/methanol: 100/0 to 97/3) to afford Exemplary Intermediate B44 (benzyl 4-hydroxy-4-((4-((3-(4-methoxy-3-(pentyloxy)phenyl)-2-oxotetrahydropyrimidin-1(2H)-yl)methyl)-1H-indol-1-yl)methyl)piperidine-1-carboxylate).

The following compounds are prepared in a similar manner as Exemplary Intermediate B44, as described above.

| Heterocycle | Epoxide | Compound |
|---|---|---|
| 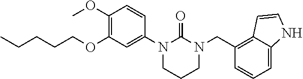 [SMILES: CCCCCOc1cc(N2CCCN(Cc3cccc4[nH]ccc34)C2=O)ccc1OC] Exemplary Intermediate B21 | 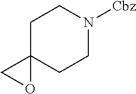 [SMILES: O=C(OCc1ccccc1)N1CCC2(CC1)CO2] | 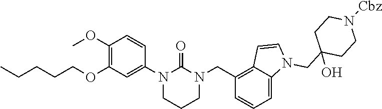 [SMILES: CCCCCOc1cc(N2CCCN(Cc3cccc4c3ccn4CC3(O)CCN(C(=O)OCc4ccccc4)CC3)C2=O)ccc1OC] |
| | | Exemplary Intermediate B44 |
| 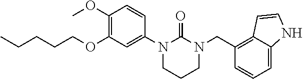 [SMILES: CCCCCOc1cc(N2CCCN(Cc3cccc4[nH]ccc34)C2=O)ccc1OC] | 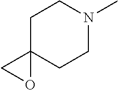 | 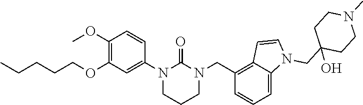 [SMILES: CCCCCOc1cc(N2CCCN(Cc3cccc4c3ccn4CC3(O)CCN(C)CC3)C2=O)ccc1OC] |

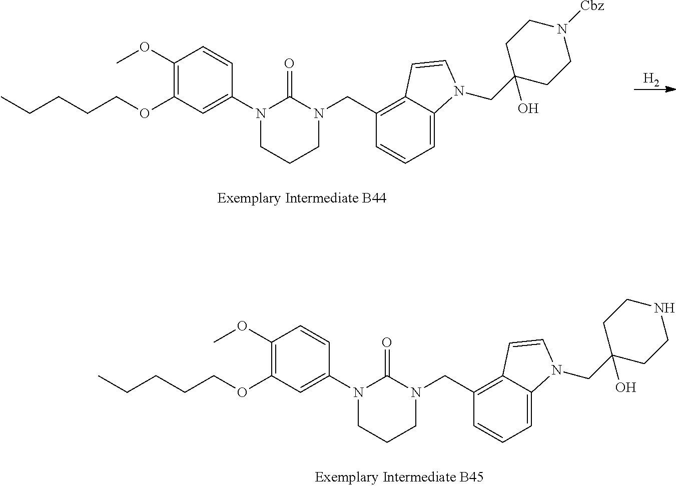 [SMILES: CCCCCOc1cc(N2CCCN(Cc3cccc4c3ccn4CC3(O)CCN(C(=O)OCc4ccccc4)CC3)C2=O)ccc1OC ; "H2" ; CCCCCOc1cc(N2CCCN(Cc3cccc4c3ccn4CC3(O)CCNCC3)C2=O)ccc1OC]

A solution of Exemplary Intermediate B44 (benzyl 4-hydroxy-4-((4-((3-(4-methoxy-3-(pentyloxy)phenyl)-2-oxotetrahydropyrimidin-1(2H)-yl)methyl)-1H-indol-1-yl)methyl) piperidine-1-carboxylate; 100 mg, 0.045 mmol) in methanol (2 mL) is treated with 10 wt % palladium on carbon (5 mg, 0.005 mmol). The reaction flask is purged with hydrogen then stirred at room temperature under a hydrogen atmosphere for 4 h. The catalyst is filtered off, and the solvent is removed in vacuo. The residue is purified by preparative HPLC (Waters XBridge C18 OBD column, 19×150 mm, 5 μm, 5% to 100% v/v acetonitrile in water with 0.1% formic acid modifier over 45 minutes at 42 mL/min flow rate) to afford Exemplary Intermediate B45 (1-((1-((4-hydroxypiperidin-4-yl)methyl)-1H-indol-4-yl)methyl)-3-(4-methoxy-3-(pentyloxy)phenyl)tetrahydropyrimidin-2(1H)-one; 68 mg, 85% yield).

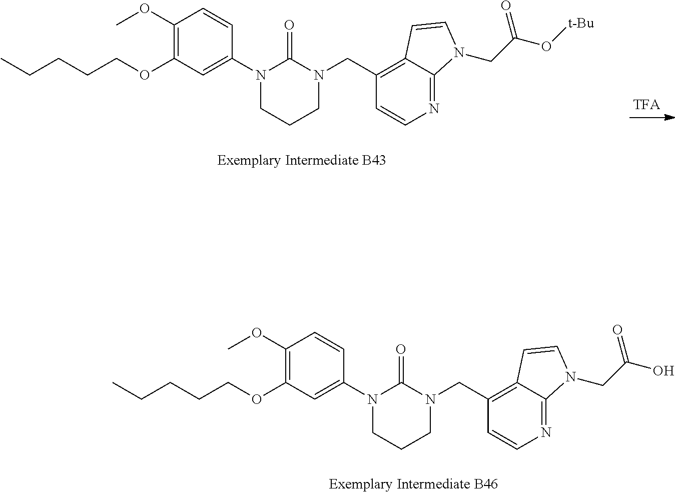 [SMILES: CCCCCOc1cc(N2CCCN(Cc3ccnc4c3ccn4CC(=O)OC(C)(C)C)C2=O)ccc1OC ; CCCCCOc1cc(N2CCCN(Cc3ccnc4c3ccn4CC(=O)O)C2=O)ccc1OC]

Trifluoroacetic acid (5.1 mL) is added to Exemplary Intermediate B43 (tert-butyl 2-(4-((3-(4-methoxy-3-(pentyloxy)phenyl)-2-oxotetrahydropyrimidin-1(2H)-yl)methyl)-1H-pyrrolo[2,3-b]pyridin-1-yl)acetate) at 0° C. The reaction mixture is stirred at 0° C. for 10 min, then warmed to room temperature and stirred for 2 h. The volatiles are removed in vacuo. Diethyl ether is added to the residue, and the resulting suspension is sonicated. The ether is decanted, and the remaining solids are further triturated with methanol to afford Exemplary Intermediate B46 (2-(4-((3-(4-methoxy-3-(pentyloxy)phenyl)-2-oxotetrahydropyrimidin-1(2H)-yl)methyl)-1H-pyrrolo[2,3-b]pyridin-1-yl)acetic acid).

The following compounds are prepared in a similar manner as Exemplary Intermediate B46, as described above.

| Heterocycle | Compound |
|---|---|
| 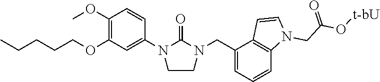 | 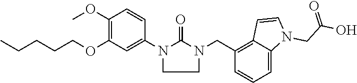 [SMILES: CCCCCOc1cc(N2CCN(Cc3cccc4c3ccn4CC(=O)O)C2=O)ccc1OC] |
| 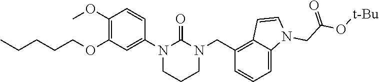 [SMILES: CCCCCOc1cc(N2CCCN(Cc3cccc4c3ccn4CC(=O)OC(C)(C)C)C2=O)ccc1OC] | 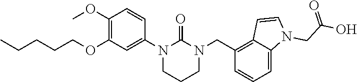 [SMILES: CCCCCOc1cc(N2CCCN(Cc3cccc4c3ccn4CC(=O)O)C2=O)ccc1OC] |
| 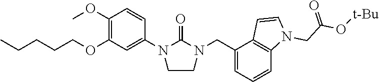 [SMILES: CCCCCOc1cc(N2CCN(Cc3cccc4c3ccn4CC(=O)OC(C)(C)C)C2=O)ccc1OC] | 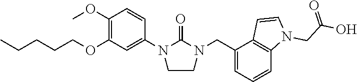 [SMILES: CCCCCOc1cc(N2CCN(Cc3cccc4c3ccn4CC(=O)O)C2=O)ccc1OC] |
| 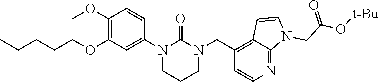 [SMILES: CCCCCOc1cc(N2CCCN(Cc3ccnc4c3ccn4CC(=O)OC(C)(C)C)C2=O)ccc1OC] Exemplary Intermediate B43 | 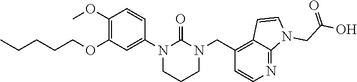 [SMILES: CCCCCOc1cc(N2CCCN(Cc3ccnc4c3ccn4CC(=O)O)C2=O)ccc1OC] Exemplary Intermediate B46 |
| 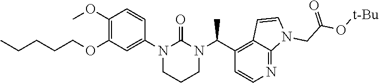 [SMILES: CCCCCOc1cc(N2CCCN([C@@H](C)c3ccnc4c3ccn4CC(=O)OC(C)(C)C)C2=O)ccc1OC] | 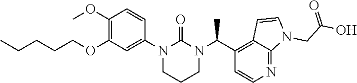 [SMILES: CCCCCOc1cc(N2CCCN([C@@H](C)c3ccnc4c3ccn4CC(=O)O)C2=O)ccc1OC] |
| 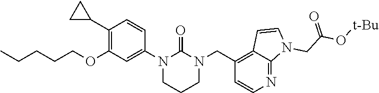 [SMILES: CCCCCOc1cc(N2CCCN(Cc3ccnc4c3ccn4CC(=O)OC(C)(C)C)C2=O)ccc1C1CC1] | 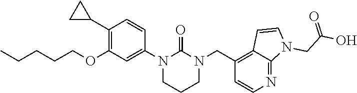 [SMILES: CCCCCOc1cc(N2CCCN(Cc3ccnc4c3ccn4CC(=O)O)C2=O)ccc1C1CC1] |
| 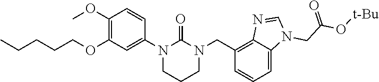 [SMILES: CCCCCOc1cc(N2CCCN(Cc3cccc4c3ncn4CC(=O)OC(C)(C)C)C2=O)ccc1OC] | 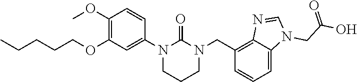 [SMILES: CCCCCOc1cc(N2CCCN(Cc3cccc4c3ncn4CC(=O)O)C2=O)ccc1OC] |

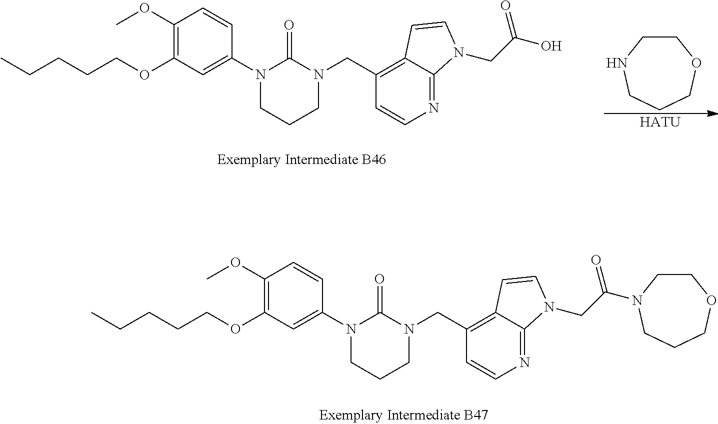

Exemplary Intermediate B46 (2-(4-((3-(4-methoxy-3-(pentyloxy)phenyl)-2-oxotetrahydropyrimidin-1(2H)-yl)methyl)-1H-pyrrolo[2,3-b]pyridin-1-yl)acetic acid) is dissolved in N,N-dimethylformamide, then HATU, 1,4-oxazepane, and N,N-diisopropylethylamine are added. Reaction mixture is stirred at room temperature for 2 h. Water is added to reaction mixture, and is extracted three times with ethyl acetate. The organic layers are dried over a phase separator and concentrated in vacuo. The residue is purified by column chromatography over silica (dichloromethane/methanol: 100/0 to 97/3) to afford Exemplary Intermediate B47 (1-((1-(2-(1,4-oxazepan-4-yl)-2-oxoethyl)-1H-pyrrolo[2,3-b]pyridin-4-yl)methyl)-3-(4-methoxy-3-(pentyloxy)phenyl)tetrahydropyrimidin-2(1H)-one).

The following compounds are prepared in a similar manner as Exemplary Intermediate B47, as described above.

| Heterocycle | Bromide | Compound |
|---|---|---|
| 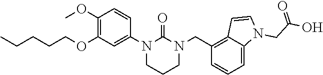 [SMILES: CCCCCOc1cc(N2CCCN(Cc3cccc4c3ccn4CC(=O)O)C2=O)ccc1OC] | 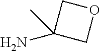 | 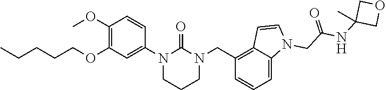 [SMILES: CCCCCOc1cc(N2CCCN(Cc3cccc4c3ccn4CC(=O)NC3(C)COC3)C2=O)ccc1OC] |
| 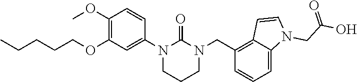 [SMILES: CCCCCOc1cc(N2CCCN(Cc3cccc4c3ccn4CC(=O)O)C2=O)ccc1OC] | 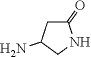 | 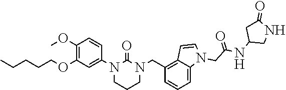 [SMILES: CCCCCOc1cc(N2CCCN(Cc3cccc4c3ccn4CC(=O)NC3CNC(=O)C3)C2=O)ccc1OC] |
| 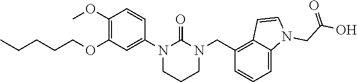 [SMILES: CCCCCOc1cc(N2CCCN(Cc3cccc4c3ccn4CC(=O)O)C2=O)ccc1OC] | 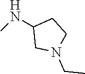 | 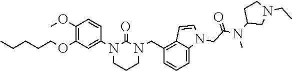 [SMILES: CCCCCOc1cc(N2CCCN(Cc3cccc4c3ccn4CC(=O)N(C)C3CCN(CC)C3)C2=O)ccc1OC] |
| 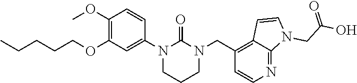 [SMILES: CCCCCOc1cc(N2CCCN(Cc3ccnc4c3ccn4CC(=O)O)C2=O)ccc1OC] | 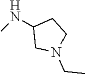 | 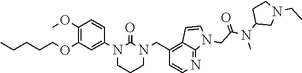 [SMILES: CCCCCOc1cc(N2CCCN(Cc3ccnc4c3ccn4CC(=O)N(C)C3CCN(CC)C3)C2=O)ccc1OC] |
| 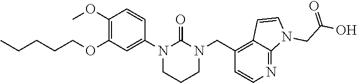 [SMILES: CCCCCOc1cc(N2CCCN(Cc3ccnc4c3ccn4CC(=O)O)C2=O)ccc1OC] | 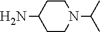 | 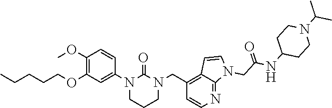 [SMILES: CCCCCOc1cc(N2CCCN(Cc3ccnc4c3ccn4CC(=O)NC3CCN(C(C)C)CC3)C2=O)ccc1OC] |
| 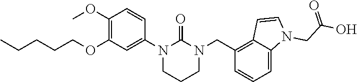 [SMILES: CCCCCOc1cc(N2CCCN(Cc3cccc4c3ccn4CC(=O)O)C2=O)ccc1OC] | 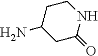 | 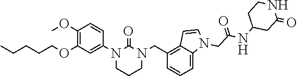 [SMILES: CCCCCOc1cc(N2CCCN(Cc3cccc4c3ccn4CC(=O)NC3CCNC(=O)C3)C2=O)ccc1OC] |
| 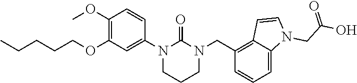 [SMILES: CCCCCOc1cc(N2CCCN(Cc3cccc4c3ccn4CC(=O)O)C2=O)ccc1OC] | 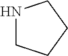 | 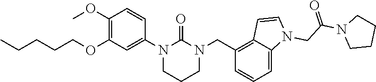 [SMILES: CCCCCOc1cc(N2CCCN(Cc3cccc4c3ccn4CC(=O)N3CCCC3)C2=O)ccc1OC] |
| 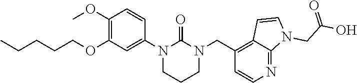 [SMILES: CCCCCOc1cc(N2CCCN(Cc3ccnc4c3ccn4CC(=O)O)C2=O)ccc1OC] | 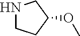 | 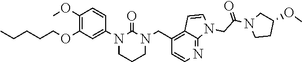 [SMILES: CCCCCOc1cc(N2CCCN(Cc3ccnc4c3ccn4CC(=O)N3CC[C@@H](OC)C3)C2=O)ccc1OC] |
| 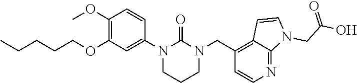 [SMILES: CCCCCOc1cc(N2CCCN(Cc3ccnc4c3ccn4CC(=O)O)C2=O)ccc1OC] | 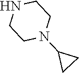 | 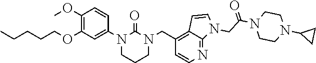 [SMILES: CCCCCOc1cc(N2CCCN(Cc3ccnc4c3ccn4CC(=O)N3CCN(C4CC4)CC3)C2=O)ccc1OC] |
| 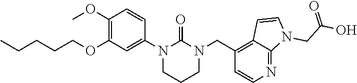 [SMILES: CCCCCOc1cc(N2CCCN(Cc3ccnc4c3ccn4CC(=O)O)C2=O)ccc1OC] | 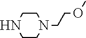 | 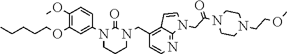 [SMILES: CCCCCOc1cc(N2CCCN(Cc3ccnc4c3ccn4CC(=O)N3CCN(CCOC)CC3)C2=O)ccc1OC] |
| 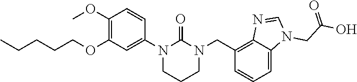 [SMILES: CCCCCOc1cc(N2CCCN(Cc3cccc4c3ncn4CC(=O)O)C2=O)ccc1OC] | 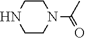 | 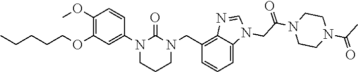 [SMILES: CCCCCOc1cc(N2CCCN(Cc3cccc4c3ncn4CC(=O)N3CCN(C(C)=O)CC3)C2=O)ccc1OC] |
| 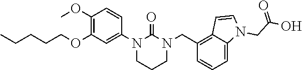 [SMILES: CCCCCOc1cc(N2CCCN(Cc3cccc4c3ccn4CC(=O)O)C2=O)ccc1OC] | 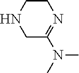 | 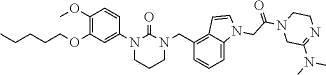 [SMILES: CCCCCOc1cc(N2CCCN(Cc3cccc4c3ccn4CC(=O)N3CCN=C(N(C)C)C3)C2=O)ccc1OC] |
| 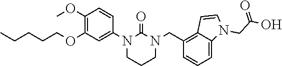 [SMILES: CCCCCOc1cc(N2CCCN(Cc3cccc4c3ccn4CC(=O)O)C2=O)ccc1OC] | 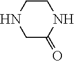 | 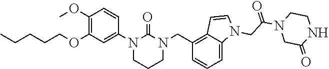 [SMILES: CCCCCOc1cc(N2CCCN(Cc3cccc4c3ccn4CC(=O)N3CCNC(=O)C3)C2=O)ccc1OC] |
| 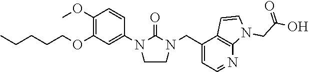 [SMILES: CCCCCOc1cc(N2CCN(Cc3ccnc4c3ccn4CC(=O)O)C2=O)ccc1OC] | 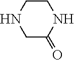 | 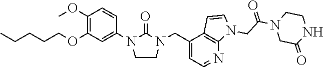 [SMILES: CCCCCOc1cc(N2CCN(Cc3ccnc4c3ccn4CC(=O)N3CCNC(=O)C3)C2=O)ccc1OC] |
| 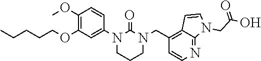 [SMILES: CCCCCOc1cc(N2CCCN(Cc3ccnc4c3ccn4CC(=O)O)C2=O)ccc1OC] | 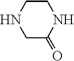 | 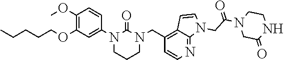 [SMILES: CCCCCOc1cc(N2CCCN(Cc3ccnc4c3ccn4CC(=O)N3CCNC(=O)C3)C2=O)ccc1OC] |
| 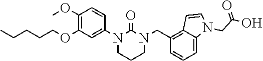 [SMILES: CCCCCOc1cc(N2CCCN(Cc3cccc4c3ccn4CC(=O)O)C2=O)ccc1OC] | 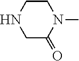 | 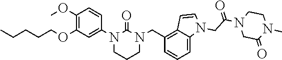 [SMILES: CCCCCOc1cc(N2CCCN(Cc3cccc4c3ccn4CC(=O)N3CCN(C)C(=O)C3)C2=O)ccc1OC] |
| 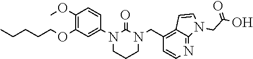 [SMILES: CCCCCOc1cc(N2CCCN(Cc3ccnc4c3ccn4CC(=O)O)C2=O)ccc1OC] | 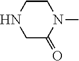 | 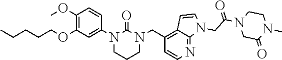 [SMILES: CCCCCOc1cc(N2CCCN(Cc3ccnc4c3ccn4CC(=O)N3CCN(C)C(=O)C3)C2=O)ccc1OC] |
| 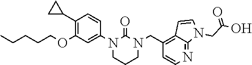 [SMILES: CCCCCOc1cc(N2CCCN(Cc3ccnc4c3ccn4CC(=O)O)C2=O)ccc1C1CC1] | 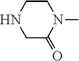 | 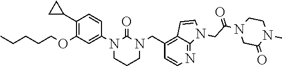 [SMILES: CCCCCOc1cc(N2CCCN(Cc3ccnc4c3ccn4CC(=O)N3CCN(C)C(=O)C3)C2=O)ccc1C1CC1] |
| 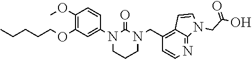 [SMILES: CCCCCOc1cc(N2CCCN(Cc3ccnc4c3ccn4CC(=O)O)C2=O)ccc1OC] | 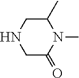 | 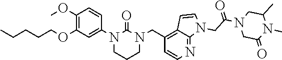 [SMILES: CCCCCOc1cc(N2CCCN(Cc3ccnc4c3ccn4CC(=O)N3CC(=O)N(C)C(C)C3)C2=O)ccc1OC] |
| 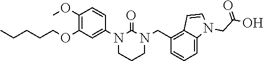 [SMILES: CCCCCOc1cc(N2CCCN(Cc3cccc4c3ccn4CC(=O)O)C2=O)ccc1OC] | 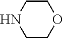 | 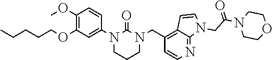 [SMILES: CCCCCOc1cc(N2CCCN(Cc3ccnc4c3ccn4CC(=O)N3CCOCC3)C2=O)ccc1OC] |
| 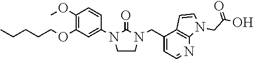 [SMILES: CCCCCOc1cc(N2CCN(Cc3ccnc4c3ccn4CC(=O)O)C2=O)ccc1OC] | 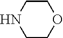 | 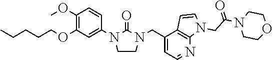 [SMILES: CCCCCOc1cc(N2CCN(Cc3ccnc4c3ccn4CC(=O)N3CCOCC3)C2=O)ccc1OC] |
| 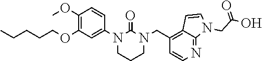 [SMILES: CCCCCOc1cc(N2CCCN(Cc3ccnc4c3ccn4CC(=O)O)C2=O)ccc1OC] | 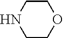 | 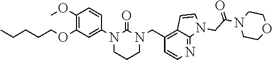 [SMILES: CCCCCOc1cc(N2CCCN(Cc3ccnc4c3ccn4CC(=O)N3CCOCC3)C2=O)ccc1OC] |
| 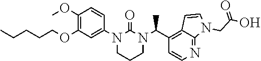 [SMILES: CCCCCOc1cc(N2CCCN([C@@H](C)c3ccnc4c3ccn4CC(=O)O)C2=O)ccc1OC] | 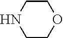 | 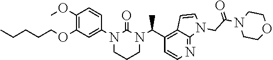 [SMILES: CCCCCOc1cc(N2CCCN([C@@H](C)c3ccnc4c3ccn4CC(=O)N3CCOCC3)C2=O)ccc1OC] |
| 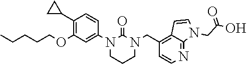 [SMILES: CCCCCOc1cc(N2CCCN(Cc3ccnc4c3ccn4CC(=O)O)C2=O)ccc1C1CC1] | 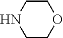 | 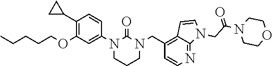 [SMILES: CCCCCOc1cc(N2CCCN(Cc3ccnc4c3ccn4CC(=O)N3CCOCC3)C2=O)ccc1C1CC1] |
| 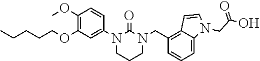 [SMILES: CCCCCOc1cc(N2CCCN(Cc3cccc4c3ccn4CC(=O)O)C2=O)ccc1OC] | 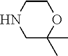 | 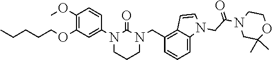 [SMILES: CCCCCOc1cc(N2CCCN(Cc3cccc4c3ccn4CC(=O)N3CCOC(C)(C)C3)C2=O)ccc1OC] |
| 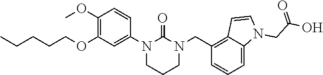 [SMILES: CCCCCOc1cc(N2CCCN(Cc3cccc4c3ccn4CC(=O)O)C2=O)ccc1OC] | 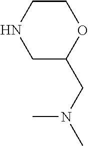 | 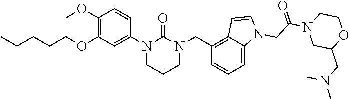 [SMILES: CCCCCOc1cc(N2CCCN(Cc3cccc4c3ccn4CC(=O)N3CCOC(CN(C)C)C3)C2=O)ccc1OC] |
| 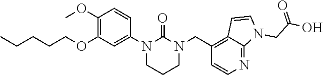 [SMILES: CCCCCOc1cc(N2CCCN(Cc3ccnc4c3ccn4CC(=O)O)C2=O)ccc1OC] | 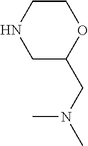 | 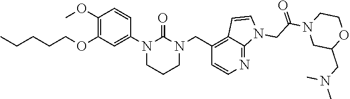 [SMILES: CCCCCOc1cc(N2CCCN(Cc3ccnc4c3ccn4CC(=O)N3CCOC(CN(C)C)C3)C2=O)ccc1OC] |
| 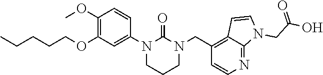 [SMILES: CCCCCOc1cc(N2CCCN(Cc3ccnc4c3ccn4CC(=O)O)C2=O)ccc1OC] | 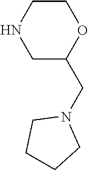 [SMILES: C1CCN(CC2CNCCO2)C1] | 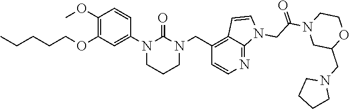 [SMILES: CCCCCOc1cc(N2CCCN(Cc3ccnc4c3ccn4CC(=O)N3CCOC(CN4CCCC4)C3)C2=O)ccc1OC] |
| 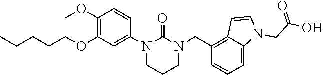 [SMILES: CCCCCOc1cc(N2CCCN(Cc3cccc4c3ccn4CC(=O)O)C2=O)ccc1OC] | 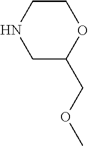 | 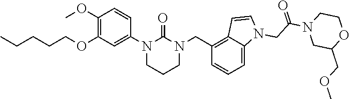 [SMILES: CCCCCOc1cc(N2CCCN(Cc3cccc4c3ccn4CC(=O)N3CCOC(COC)C3)C2=O)ccc1OC] |
| 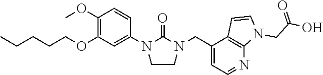 [SMILES: CCCCCOc1cc(N2CCN(Cc3ccnc4c3ccn4CC(=O)O)C2=O)ccc1OC] | 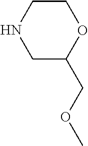 | 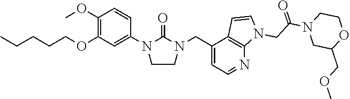 [SMILES: CCCCCOc1cc(N2CCN(Cc3ccnc4c3ccn4CC(=O)N3CCOC(COC)C3)C2=O)ccc1OC] |
| 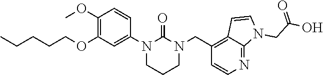 [SMILES: CCCCCOc1cc(N2CCCN(Cc3ccnc4c3ccn4CC(=O)O)C2=O)ccc1OC] | 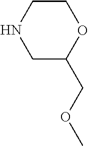 | 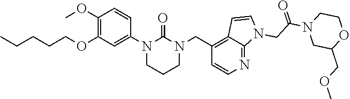 [SMILES: CCCCCOc1cc(N2CCCN(Cc3ccnc4c3ccn4CC(=O)N3CCOC(COC)C3)C2=O)ccc1OC] |
| 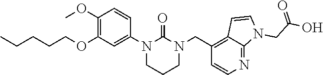 [SMILES: CCCCCOc1cc(N2CCCN(Cc3ccnc4c3ccn4CC(=O)O)C2=O)ccc1OC] | 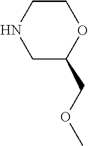 | 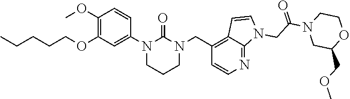 [SMILES: CCCCCOc1cc(N2CCCN(Cc3ccnc4c3ccn4CC(=O)N3CCO[C@@H](COC)C3)C2=O)ccc1OC] |
| 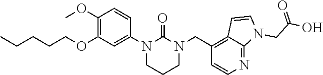 [SMILES: CCCCCOc1cc(N2CCCN(Cc3ccnc4c3ccn4CC(=O)O)C2=O)ccc1OC] | 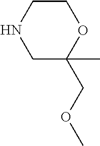 | 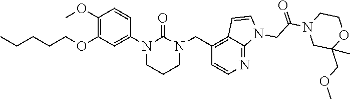 [SMILES: CCCCCOc1cc(N2CCCN(Cc3ccnc4c3ccn4CC(=O)N3CCOC(C)(COC)C3)C2=O)ccc1OC] |
| 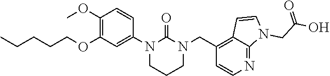 [SMILES: CCCCCOc1cc(N2CCCN(Cc3ccnc4c3ccn4CC(=O)O)C2=O)ccc1OC] | 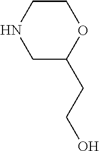 | 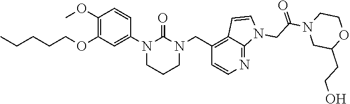 [SMILES: CCCCCOc1cc(N2CCCN(Cc3ccnc4c3ccn4CC(=O)N3CCOC(CCO)C3)C2=O)ccc1OC] |
| 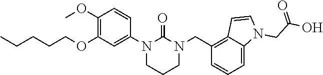 [SMILES: CCCCCOc1cc(N2CCCN(Cc3cccc4c3ccn4CC(=O)O)C2=O)ccc1OC] | 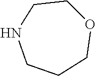 | 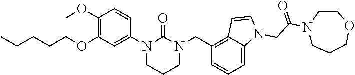 [SMILES: CCCCCOc1cc(N2CCCN(Cc3cccc4c3ccn4CC(=O)N3CCCOCC3)C2=O)ccc1OC] |
| 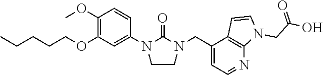 [SMILES: CCCCCOc1cc(N2CCN(Cc3ccnc4c3ccn4CC(=O)O)C2=O)ccc1OC] | 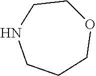 | 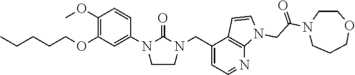 [SMILES: CCCCCOc1cc(N2CCN(Cc3ccnc4c3ccn4CC(=O)N3CCCOCC3)C2=O)ccc1OC] |
| 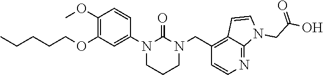 [SMILES: CCCCCOc1cc(N2CCCN(Cc3ccnc4c3ccn4CC(=O)O)C2=O)ccc1OC] | 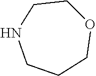 | 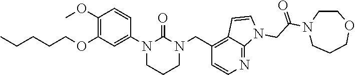 [SMILES: CCCCCOc1cc(N2CCCN(Cc3ccnc4c3ccn4CC(=O)N3CCCOCC3)C2=O)ccc1OC] |
| 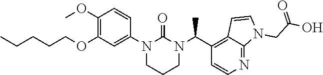 [SMILES: CCCCCOc1cc(N2CCCN([C@@H](C)c3ccnc4c3ccn4CC(=O)O)C2=O)ccc1OC] | 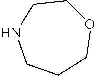 | 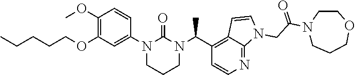 [SMILES: CCCCCOc1cc(N2CCCN([C@@H](C)c3ccnc4c3ccn4CC(=O)N3CCCOCC3)C2=O)ccc1OC] |
| 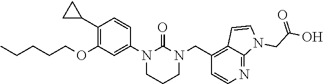 [SMILES: CCCCCOc1cc(N2CCCN(Cc3ccnc4c3ccn4CC(=O)O)C2=O)ccc1C1CC1] | 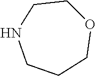 | 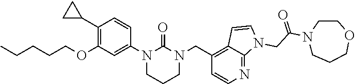 [SMILES: CCCCCOc1cc(N2CCCN(Cc3ccnc4c3ccn4CC(=O)N3CCCOCC3)C2=O)ccc1C1CC1] |

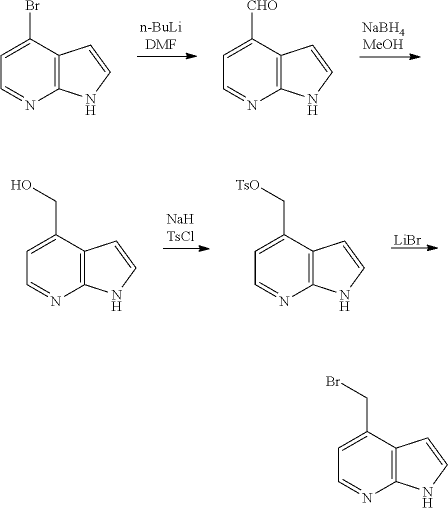

n-Butyllithium (1.6 M solution in hexane, 127 mL, 0.204 mol) is added dropwise to a solution of 4-bromo-1H-pyrrolo[2,3-b]pyridine (20.0 g, 0.102 mmol) in anhydrous tetrahydrofuran (500 mL) at −70° C. over 30 min. The reaction mixture is stirred for 1 hour at this temperature, then a solution of N,N-dimethylformamide (22 g, 0.3 mol) in anhydrous tetrahydrofuran (100 mL) is added. The mixture is stirred for 0.5 h at −70° C., then allowed to warm to room temperature. The mixture is quenched carefully with ice-water (500 mL), then extracted with ethyl acetate (3×300 mL). The combined organic phases are washed with brine (3×300 mL), dried over anhydrous sodium sulfate, filtered, and concentrated in vacuo. The residue is triturated with ethyl acetate/hexane (1:4, 100 mL) to afford 1H-pyrrolo[2,3-b]pyridine-4-carbaldehyde (8.1 g).

Sodium borohydride (5.2 g, 137 mmol) is added portionwise to a solution of 1H-pyrrolo[2,3-b]pyridine-4-carbaldehyde (10.0 g, 68 mmol) in methanol (100 mL) at 0-10° C. Then the mixture is stirred 2 h at room temperature. The mixture is quenched with water (100 mL), and the organic solvent is removed under reduced pressure, the residue is extracted with ethyl acetate (3×100 mL). The combined organic phases are washed with brine (3×100 mL), then dried over anhydrous sodium sulfate, filtered, and concentrated in vacuo. The residue is triturated with ethyl acetate/hexane (1:4, 50 mL), to afford (1H-pyrrolo[2,3-b]pyridin-4-yl)methanol (8.4 g).

Sodium hydride (60 wt % in oil, 3.6 g, 89 mmol) is added portionwise to a solution of (1H-pyrrolo[2,3-b]pyridin-4-yl)methanol (6.3 g, 42.6 mmol) in anhydrous tetrahydrofuran (100 mL) at 0° C. over 10 min. The reaction mixture is stirred for 0.5 hours at room temperature, then re-cooled to 0° C. p-Toluenesulfonyl chloride (17.0 g, 89 mmol) is added. The reaction mixture is warmed to room temperature and stirred for 2 hours. The mixture is quenched with water (200 mL) then extracted with ethyl acetate (3×100 mL). The combined organic phases are washed with brine (3×100 mL), then dried over anhydrous sodium sulfate, filtered, and concentrated in vacuo. The residue is triturated with ethyl acetate/hexane (1:10, 50 mL) to afford (1-tosyl-1H-pyrrolo[2,3-b]pyridin-4-yl)methyl 4-methylbenzenesulfonate (10.5 g).

A mixture of (1-tosyl-1H-pyrrolo[2,3-b]pyridin-4-yl)methyl 4-methylbenzenesulfonate (5.1 g, 11.2 mmol) and lithium bromide (2.7 g, 14.5 mmol) in anhydrous tetrahydrofuran (50 mL) is stirred at room temperature for 4 hours. The mixture is quenched with water (100 mL) then extracted with ethyl acetate (3×100 mL). The combined organic phases are washed with brine (3×100 mL), then dried over anhydrous sodium sulfate, filtered, and concentrated in vacuo to afford 4-(bromomethyl)-1-tosyl-1H-pyrrolo[2,3-b]pyridine (4.0 g).

### Example 18. Compound Analysis by Surface Plasmon Resonance (SPR)

C5 inhibitor candidate compounds were synthesized according to standard methods known in the art [see, e.g. Morrison and Boyd in “Organic Chemistry”, 6^{th} edition, Prentice Hall (1992)], and analyzed using surface plasmon resonance (SPR) technology to generate data on the affinity, specificity, and kinetics of compound interactions with human C5 complement protein in real time without the need for labeling.

SensiQ FE SPR system (SensiQ Technologies, Oklahoma City, Okla.) was used to provide sensitive and accurate detection of binding of small molecules to the very large C5 protein (MW=195,000 Da). The chip was prepared by preconditioning the sensor according to the protocol of the SensiQ FE using 10 mM HCl, 50 mM NAOH and 0.1% SDS. The sensor chip was activated by using a mixture of fresh EDAC (1-ethyl-3-(-3-dimethylaminopropyl) carbodiimide) (Sigma Co., St. Louis, Mo.) and NHS (N-hydroxy succinimide) (Sigma Co., St. Louis, Mo.). Human C5 was surface immobilized to a Pioneer Biosensor chip via random amine coupling (>12,000 RU) which makes use of the N-terminus and ε-amino groups of lysine residues of the protein ligand. Immobilization was done by injecting 30-40 ug/ml C5 in 10 mM NaAc pH 4.5 onto designated channels at a rate of 10 μL/minute for 12 minutes, targeting RL>12000 RU for small molecules.

Compounds were diluted in DMSO in a format of 100-fold final concentration and 3-fold serial dilution (5 or 6 dilutions). The 100-fold compounds were transferred to one fold DMSO-free assay buffer in the 96-well test plate. The compound solution was injected at a rate of 60 μL/minute for 30-60 seconds, followed by 60-90 seconds dissociation time, buffer flushing and/or priming. Blank solution (1% DMSO assay buffer) was run for every 6 injections of compounds. Double reference by subtracting both blank channel and reference channel was applied for data processing. Titration of C5 binding compounds to the C5-immobilized biosensor chip surface led to interactions between C5 and potential binders, and the resulting changes of surface refractive index were sensitively measured by the system.

SPR data was analyzed with the managing software provided by SensiQ and equilibrium dissociation constant (K_{D}) values were determined for each compound at 37° C. Values obtained are presented in Table 24. Where a range of compound concentrations were analyzed, the lowest value obtained is presented.

TABLE 24
Surface plasmon resonance (SPR) data

| | K_{D} |
|---|---|
| Compound No. | (nM) |
| CU0508 | 0.009 |
| CU0543 | 0.016 |
| CU0708 | 0.018 |
| CU0565 | 0.024 |
| CU0550 | 0.031 |
| CU0524 | 0.038 |
| CU0537 | 0.039 |
| CU0669 | 0.041 |
| CU0527 | 0.041 |
| CU0530 | 0.051 |
| CU0526 | 0.052 |
| CU0535 | 0.053 |
| CU0560 | 0.054 |
| CU0533 | 0.056 |
| CU0528 | 0.057 |
| CU0509 | 0.069 |
| CU0572 | 0.074 |
| CU0699 | 0.078 |
| CU0514 | 0.093 |
| CU0638 | 0.094 |
| CU0597 | 0.115 |
| CU0567 | 0.118 |
| CU0601 | 0.133 |
| CU0561 | 0.140 |
| CU0516 | 0.150 |
| CU0558 | 0.152 |
| CU0540 | 0.158 |
| CU0610 | 0.207 |
| CU0576 | 0.221 |
| CU0718 | 0.245 |
| CU0504 | 0.254 |
| CU0671 | 0.275 |
| CU0519 | 0.291 |
| CU0589 | 0.302 |
| CU0575 | 0.304 |
| CU0541 | 0.310 |
| CU0515 | 0.320 |
| CU0502 | 0.320 |
| CU0529 | 0.322 |
| CU0534 | 0.363 |
| CU0511 | 0.470 |
| CU0571 | 0.481 |
| CU0562 | 0.498 |
| CU0613 | 0.533 |
| CU0686 | 0.618 |
| CU0598 | 0.747 |
| CU0518 | 0.820 |
| CU0675 | 0.860 |
| CU0632 | 0.880 |
| CU0626 | 1.043 |
| CU0555 | 1.060 |
| CU0644 | 1.110 |
| CU0662 | 1.154 |
| CU0500 | 1.203 |
| CU0657 | 1.324 |
| CU0695 | 1.351 |
| CU0690 | 1.689 |
| CU0681 | 2.195 |
| CU0704 | 2.320 |
| CU0668 | 2.532 |
| CU0737 | 8.100 |

### Example 19. Compound Analysis by Red Blood Cell (RBC) Hemolysis Assay

Sheep red blood cells coated with rabbit anti-sheep erythrocyte antiserum (EA cells; Complement Technology, Tyler, Tex.) were used to assay compound inhibitory activity of the classical complement activation pathway. Briefly, the EA cells were washed once and resuspended in the same volume of GVB++ buffer (Complement Technology, Tyler, Tex.). 25 μL of EA cells were then distributed into each well of 384-well tissue culture plates using Apricot iPipette Pro (Apricot Designs; Covina, Calif.). Compounds were tested in 10 points of final concentrations ranging from 16.67 μM to 1.65 μM in a 6-fold titration series. Compounds were dispensed into 384-well plates from 6.7 mM and 3.35 μM DMSO working stocks using an HP Digital Dispenser (HP; Corvallis, Oreg.). The reactions also contained 1.5% (v/v) C5-depleted human serum (Complement Technology). Hemolysis was induced by addition of human C5 (Complement Technology) at a concentration of 0.5 nM and plates were incubated for 1 hour at 37° C. in a cell culture incubator. The extent of hemolysis was measured by ability of released hemoglobin to catalyze luminol in the presence of hydrogen peroxide. Luminescence was then measured using a plate reader.

Luminescence measurements were used to prepare a dose-response curve. From the curve, the half maximal inhibitory concentration (IC₅₀) for each compound was determined, where the IC₅₀ represents the concentration of each compound needed to reduce red blood cell hemolysis by half. Compounds with IC₅₀ values below 5 μM are presented in Table 25.

TABLE 25
Red blood cell (RBC) hemolysis assay data

| | IC₅₀ |
|---|---|
| Compound No. | (nM) |
| CU0500 | 0.7 |
| CU0501 | 0.8 |
| CU0502 | 0.8 |
| CU0503 | 0.8 |
| CU0504 | 0.8 |
| CU0505 | 0.8 |
| CU0506 | 0.8 |
| CU0507 | 0.8 |
| CU0508 | 0.8 |
| CU0509 | 0.8 |
| CU0510 | 0.9 |
| CU0511 | 0.9 |
| CU0512 | 0.9 |
| CU0513 | 0.9 |
| CU0514 | 0.9 |
| CU0515 | 0.9 |
| CU0516 | 0.9 |
| CU0517 | 0.9 |
| CU0518 | 0.9 |
| CU0519 | 0.9 |
| CU0520 | 0.9 |
| CU0521 | 1.0 |
| CU0522 | 1.0 |
| CU0523 | 1.0 |
| CU0524 | 1.0 |
| CU0525 | 1.0 |
| CU0526 | 1.0 |
| CU0527 | 1.0 |
| CU0528 | 1.0 |
| CU0529 | 1.0 |
| CU0530 | 1.0 |
| CU0531 | 1.0 |
| CU0532 | 1.1 |
| CU0533 | 1.1 |
| CU0534 | 1.1 |
| CU0535 | 1.1 |
| CU0536 | 1.1 |
| CU0537 | 1.1 |
| CU0538 | 1.1 |
| CU0539 | 1.1 |
| CU0540 | 1.1 |
| CU0541 | 1.1 |
| CU0542 | 1.1 |
| CU0543 | 1.1 |
| CU0544 | 1.1 |
| CU0545 | 1.1 |
| CU0546 | 1.2 |
| CU0547 | 1.2 |
| CU0548 | 1.2 |
| CU0549 | 1.2 |
| CU0550 | 1.2 |
| CU0551 | 1.2 |
| CU0552 | 1.2 |
| CU0553 | 1.3 |
| CU0554 | 1.3 |
| CU0555 | 1.3 |
| CU0556 | 1.3 |
| CU0557 | 1.3 |
| CU0558 | 1.3 |
| CU0559 | 1.3 |
| CU0560 | 1.3 |
| CU0561 | 1.3 |
| CU0562 | 1.4 |
| CU0563 | 1.4 |
| CU0564 | 1.4 |
| CU0565 | 1.5 |
| CU0566 | 1.5 |
| CU0567 | 1.5 |
| CU0568 | 1.5 |
| CU0569 | 1.5 |
| CU0570 | 1.6 |
| CU0571 | 1.6 |
| CU0572 | 1.6 |
| CU0573 | 1.6 |
| CU0574 | 1.7 |
| CU0575 | 1.7 |
| CU0576 | 1.7 |
| CU0577 | 1.7 |
| CU0578 | 1.8 |
| CU0579 | 1.8 |
| CU0580 | 1.8 |
| CU0581 | 1.8 |
| CU0582 | 1.8 |
| CU0583 | 1.9 |
| CU0584 | 1.9 |
| CU0585 | 1.9 |
| CU0586 | 1.9 |
| CU0587 | 2.0 |
| CU0588 | 2.0 |
| CU0589 | 2.0 |
| CU0590 | 2.0 |
| CU0591 | 2.0 |
| CU0592 | 2.0 |
| CU0593 | 2.1 |
| CU0594 | 2.1 |
| CU0595 | 2.1 |
| CU0596 | 2.1 |
| CU0597 | 2.1 |
| CU0598 | 2.1 |
| CU0599 | 2.1 |
| CU0600 | 2.1 |
| CU0601 | 2.2 |
| CU0602 | 2.2 |
| CU0603 | 2.2 |
| CU0604 | 2.3 |
| CU0605 | 2.3 |
| CU0606 | 2.3 |
| CU0607 | 2.4 |
| CU0608 | 2.4 |
| CU0609 | 2.4 |
| CU0610 | 2.4 |
| CU0611 | 2.4 |
| CU0612 | 2.4 |
| CU0613 | 2.5 |
| CU0614 | 2.5 |
| CU0615 | 2.5 |
| CU0616 | 2.5 |
| CU0617 | 2.5 |
| CU0618 | 2.6 |
| CU0619 | 2.6 |
| CU0620 | 2.6 |
| CU0621 | 2.7 |
| CU0622 | 2.7 |
| CU0623 | 2.7 |
| CU0624 | 2.9 |
| CU0625 | 3.0 |
| CU0626 | 3.0 |
| CU0627 | 3.2 |
| CU0628 | 3.2 |
| CU0629 | 3.2 |
| CU0630 | 3.2 |
| CU0631 | 3.2 |
| CU0632 | 3.3 |
| CU0633 | 3.5 |
| CU0634 | 3.6 |
| CU0635 | 3.6 |
| CU0636 | 3.6 |
| CU0637 | 3.6 |
| CU0638 | 3.6 |
| CU0639 | 3.7 |
| CU0640 | 3.7 |
| CU0641 | 3.8 |
| CU0642 | 3.8 |
| CU0643 | 3.9 |
| CU0644 | 3.9 |
| CU0645 | 3.9 |
| CU0646 | 3.9 |
| CU0647 | 4.0 |
| CU0648 | 4.0 |
| CU0649 | 4.0 |
| CU0650 | 4.1 |
| CU0651 | 4.1 |
| CU0652 | 4.3 |
| CU0653 | 4.4 |
| CU0654 | 4.4 |
| CU0655 | 4.5 |
| CU0656 | 4.6 |
| CU0657 | 4.6 |
| CU0658 | 4.8 |
| CU0659 | 4.9 |
| CU0660 | 4.9 |
| CU0661 | 5.0 |
| CU0662 | 5.1 |
| CU0663 | 5.2 |
| CU0664 | 5.3 |
| CU0665 | 5.4 |
| CU0666 | 5.4 |
| CU0667 | 5.4 |
| CU0668 | 5.5 |
| CU0669 | 5.5 |
| CU0670 | 5.6 |
| CU0671 | 6.2 |
| CU0672 | 6.2 |
| CU0673 | 6.2 |
| CU0674 | 6.4 |
| CU0675 | 6.4 |
| CU0676 | 6.5 |
| CU0677 | 6.6 |
| CU0678 | 6.6 |
| CU0679 | 6.6 |
| CU0680 | 6.7 |
| CU0681 | 6.8 |
| CU0682 | 7.0 |
| CU0683 | 7.2 |
| CU0684 | 7.3 |
| CU0685 | 7.4 |
| CU0686 | 7.9 |
| CU0687 | 7.9 |
| CU0688 | 8.0 |
| CU0689 | 8.5 |
| CU0690 | 8.7 |
| CU0691 | 8.8 |
| CU0692 | 9.1 |
| CU0693 | 9.3 |
| CU0694 | 9.6 |
| CU0695 | 9.7 |
| CU0696 | 9.7 |
| CU0697 | 10.0 |
| CU0698 | 10.1 |
| CU0699 | 10.2 |
| CU0700 | 10.3 |
| CU0701 | 10.3 |
| CU0702 | 10.5 |
| CU0703 | 10.7 |
| CU0704 | 10.8 |
| CU0705 | 10.9 |
| CU0706 | 11.1 |
| CU0707 | 11.4 |
| CU0708 | 11.6 |
| CU0709 | 11.7 |
| CU0710 | 12.1 |
| CU0711 | 12.2 |
| CU0712 | 13.1 |
| CU0713 | 13.1 |
| CU0714 | 13.3 |
| CU0715 | 13.5 |
| CU0716 | 13.8 |
| CU0717 | 14.5 |
| CU0718 | 14.7 |
| CU07I9 | 14.7 |
| CU0720 | 15.6 |
| CU0721 | 15.6 |
| CU0722 | 15.7 |
| CU0723 | 16.2 |
| CU0724 | 16.2 |
| CU0725 | 16.5 |
| CU0726 | 16.7 |
| CU0727 | 17.1 |
| CU0728 | 17.3 |
| CU0729 | 17.5 |
| CU0730 | 17.8 |
| CU0731 | 18.6 |
| CU0732 | 18.8 |
| CU0733 | 19.2 |
| CU0734 | 19.4 |
| CU0735 | 20.6 |
| CU0736 | 21.0 |
| CU0737 | 21.0 |
| CU0738 | 21.1 |
| CU0739 | 21.7 |
| CU0740 | 22.3 |
| CU0741 | 22.3 |
| CU0742 | 22.4 |
| CU0743 | 23.6 |
| CU0744 | 23.7 |
| CU0745 | 25.0 |
| CU0746 | 25.5 |
| CU0747 | 25.6 |
| CU0748 | 26.8 |
| CU0749 | 28.5 |
| CU0750 | 28.7 |
| CU0751 | 29.3 |
| CU0752 | 29.3 |
| CU0753 | 29.8 |
| CU0754 | 30.0 |
| CU0755 | 30.1 |
| CU0756 | 30.7 |
| CU0757 | 31.0 |
| CU0758 | 31.7 |
| CU0759 | 31.8 |
| CU0760 | 31.8 |
| CU0761 | 33.8 |
| CU0762 | 36.5 |
| CU0763 | 38.4 |
| CU0764 | 40.0 |
| CU0765 | 42.4 |
| CU0766 | 42.9 |
| CU0767 | 43.9 |
| CU0768 | 44.2 |
| CU0769 | 46.9 |
| CU0770 | 47.8 |
| CU0771 | 53.5 |
| CU0772 | 54.9 |
| CU0773 | 56.4 |
| CU0774 | 57.3 |
| CU0775 | 58.6 |
| CU0776 | 59.7 |
| CU0777 | 61.6 |
| CU0778 | 61.6 |
| CU0779 | 61.7 |
| CU0780 | 61.8 |
| CU0781 | 62.6 |
| CU0782 | 62.9 |
| CU0783 | 63.4 |
| CU0784 | 65.8 |
| CU0785 | 66.0 |
| CU0786 | 71.7 |
| CU0787 | 73.0 |
| CU0788 | 76.5 |
| CU0789 | 76.6 |
| CU0790 | 78.9 |
| CU0791 | 80.4 |
| CU0792 | 81.1 |
| CU0793 | 84.2 |
| CU0794 | 92.6 |
| CU0795 | 93.4 |
| CU0796 | 99.0 |
| CU0797 | 100.4 |
| CU0798 | 102.2 |
| CU0799 | 105.6 |
| CU0800 | 106.4 |
| CU0801 | 107.1 |
| CU0802 | 109.7 |
| CU0803 | 112.3 |
| CU0804 | 112.5 |
| CU0805 | 118.3 |
| CU0806 | 129.8 |
| CU0807 | 133.1 |
| CU0808 | 146.5 |
| CU0809 | 147.4 |
| CU0810 | 151.9 |
| CU0811 | 154.8 |
| CU0812 | 172.0 |
| CU0813 | 175.5 |
| CU0814 | 182.4 |
| CU0815 | 192.4 |
| CU0816 | 192.5 |
| CU0817 | 198.8 |
| CU0818 | 199.7 |
| CU0819 | 216.4 |
| CU0820 | 255.8 |
| CU0821 | 274.1 |
| CU0822 | 346.6 |
| CU0823 | 356.4 |
| CU0824 | 390.9 |
| CU0825 | 408.1 |
| CU0826 | 434.7 |
| CU0827 | 495.7 |
| CU0828 | 506.0 |
| CU0829 | 507.9 |
| CU0830 | 543.1 |
| CU0831 | 567.0 |
| CU0832 | 568.7 |
| CU0833 | 570.3 |
| CU0834 | 712.2 |
| CU0835 | 1082.8 |
| CU0836 | 1484.9 |
| CU0837 | 1842.6 |
| CU0838 | 1988.0 |
| CU0839 | 2461.4 |
| CU0840 | 2478.9 |
| CU0841 | 2574.0 |
| CU0842 | 2692.0 |
| CU0843 | 3240.7 |
| CU0844 | 3352.2 |
| CU0845 | 3544.4 |
| CU0846 | 4213.2 |
| CU0847 | 4786.8 |

### Example 20. Compound Analysis by Liquid Chromatography-Mass Spectrometry (LC-MS)

Compounds were analyzed by Liquid chromatography-mass spectrometry (LC-MS) after synthesis to confirm mass-to-charge ratio (m/Z [M+H]). Analytical LCMS was performed by Waters Aquity SDS using a linear gradient of 5% to 100% B over a 5 minute gradient, and UV visualization with a diode array detector. The column used was a C18 Aquity UPLC BEH, 2.1 mm i.d. by 50 mm length, 1.7 μM with flow rate of 0.6 ml/min. Mobile phase A was water and mobile phase B was acetonitrile (0.1% TFA). Results are shown in Table 26.

TABLE 26
LCMS assay data

| | m/z found |
|---|---|
| Compound No. | [M + H] |
| CU0833 | 401.3 |
| CU0835 | 418.8 |
| CU0822 | 420.8 |
| CU0845 | 423.2 |
| CU0844 | 426.3 |
| CU0837 | 427.1 |
| CU0825 | 435.4 |
| CU0768 | 436.0 |
| CU0808 | 438.3 |
| CU0841 | 440.2 |
| CU0715 | 440.4 |
| CU0815 | 440.8 |
| CU0759 | 441.1 |
| CU0826 | 441.1 |
| CU0842 | 442.1 |
| CU0657 | 454.0 |
| CU0784 | 454.0 |
| CU0809 | 454.5 |
| CU0612 | 457.3 |
| CU0830 | 458.2 |
| CU0736 | 459.2 |
| CU0818 | 460.8 |
| CU0787 | 460.9 |
| CU0823 | 466.2 |
| CU0773 | 467.0 |
| CU0686 | 468.0 |
| CU0708 | 468.1 |
| CU0762 | 468.2 |
| CU0763 | 468.2 |
| CU0783 | 469.2 |
| CU0750 | 469.9 |
| CU0685 | 470.0 |
| CU0738 | 470.0 |
| CU0651 | 470.4 |
| CU0623 | 470.4 |
| CU0639 | 470.6 |
| CU0796 | 471.2 |
| CU0648 | 472.0 |
| CU0816 | 472.3 |
| CU0831 | 477.0 |
| CU0834 | 479.5 |
| CU0820 | 479.8 |
| CU0758 | 480.4 |
| CU0636 | 482.0 |
| CU0770 | 482.4 |
| CU0704 | 484.0 |
| CU0579 | 485.4 |
| CU0771 | 485.4 |
| CU0720 | 486.0 |
| CU0807 | 487.8 |
| CU0587 | 493.4 |
| CU0745 | 495.3 |
| CU0802 | 496.1 |
| CU0501 | 496.2 |
| CU0617 | 496.2 |
| CU0646 | 496.2 |
| CU0748 | 496.2 |
| CU0757 | 496.2 |
| CU0624 | 496.4 |
| CU0670 | 496.5 |
| CU0611 | 498.0 |
| CU0680 | 498.0 |
| CU0695 | 498.1 |
| CU0754 | 498.1 |
| CU0633 | 498.7 |
| CU0760 | 499.8 |
| CU0576 | 501.0 |
| CU0672 | 504.2 |
| CU0655 | 504.5 |
| CU0661 | 504.9 |
| CU0608 | 505.2 |
| CU0614 | 505.2 |
| CU0628 | 505.6 |
| CU0782 | 506.3 |
| CU0679 | 507.0 |
| CU0810 | 507.2 |
| CU0829 | 508.1 |
| CU0743 | 509.0 |
| CU0791 | 509.4 |
| CU0813 | 509.4 |
| CU0668 | 510.0 |
| CU0546 | 510.2 |
| CU0613 | 510.2 |
| CU0775 | 510.2 |
| CU0555 | 511.0 |
| CU0711 | 511.1 |
| CU0618 | 511.9 |
| CU0688 | 512.0 |
| CU0691 | 512.1 |
| CU0819 | 512.1 |
| CU0828 | 512.2 |
| CU0634 | 513.9 |
| CU0578 | 514.4 |
| CU0607 | 515.0 |
| CU0583 | 515.2 |
| CU0666 | 515.3 |
| CU0795 | 517.1 |
| CU0812 | 517.1 |
| CU0801 | 518.1 |
| CU0785 | 518.3 |
| CU0793 | 518.3 |
| CU0781 | 519.1 |
| CU0644 | 519.3 |
| CU0667 | 519.3 |
| CU0761 | 520.0 |
| CU0694 | 520.4 |
| CU0839 | 521.1 |
| CU0735 | 521.4 |
| CU0662 | 522.2 |
| CU0755 | 522.2 |
| CU0687 | 522.5 |
| CU0789 | 523.3 |
| CU0843 | 524.0 |
| CU0609 | 524.1 |
| CU0621 | 524.1 |
| CU0645 | 524.1 |
| CU0832 | 524.9 |
| CU0712 | 525.7 |
| CU0698 | 526.0 |
| CU0596 | 526.2 |
| CU0653 | 527.8 |
| CU0592 | 528.4 |
| CU0637 | 530.9 |
| CU0575 | 531.2 |
| CU0799 | 532.2 |
| CU0681 | 533.5 |
| CU0664 | 533.8 |
| CU0697 | 534.1 |
| CU0702 | 534.1 |
| CU0689 | 534.2 |
| CU0764 | 534.2 |
| CU0767 | 534.3 |
| CU0737 | 534.4 |
| CU0752 | 534.4 |
| CU0747 | 534.5 |
| CU0730 | 534.5 |
| CU0798 | 534.7 |
| CU0726 | 534.9 |
| CU0722 | 535.5 |
| CU0803 | 536.0 |
| CU0749 | 536.2 |
| CU0699 | 536.3 |
| CU0718 | 536.3 |
| CU0597 | 536.4 |
| CU0642 | 536.4 |
| CU0548 | 536.5 |
| CU0584 | 536.5 |
| CU0721 | 536.5 |
| CU0786 | 536.7 |
| CU0824 | 537.0 |
| CU0777 | 537.2 |
| CU0545 | 537.5 |
| CU0573 | 537.9 |
| CU0536 | 538.1 |
| CU0552 | 538.1 |
| CU0701 | 538.2 |
| CU0778 | 538.2 |
| CU0725 | 538.3 |
| CU0776 | 538.3 |
| CU0690 | 538.5 |
| CU0671 | 538.5 |
| CU0840 | 538.5 |
| CU0753 | 538.9 |
| CU0663 | 538.9 |
| CU0588 | 539.0 |
| CU0632 | 539.0 |
| CU0769 | 539.0 |
| CU0626 | 539.2 |
| CU0585 | 540.3 |
| CU0638 | 540.3 |
| CU0602 | 540.4 |
| CU0707 | 540.5 |
| CU0649 | 540.8 |
| CU0513 | 541.4 |
| CU0674 | 541.4 |
| CU0581 | 541.8 |
| CU0673 | 542.3 |
| CU0847 | 543.2 |
| CU0558 | 543.4 |
| CU0616 | 543.4 |
| CU0734 | 543.4 |
| CU0790 | 544.2 |
| CU0713 | 544.4 |
| CU0500 | 544.5 |
| CU0514 | 545.3 |
| CU0846 | 545.5 |
| CU0780 | 546.1 |
| CU0709 | 546.3 |
| CU0740 | 546.5 |
| CU0512 | 546.5 |
| CU0650 | 547.0 |
| CU0568 | 547.5 |
| CU0507 | 547.9 |
| CU0586 | 548.0 |
| CU0542 | 548.4 |
| CU0503 | 548.4 |
| CU0505 | 548.4 |
| CU0677 | 548.6 |
| CU0537 | 549.2 |
| CU0714 | 549.2 |
| CU0756 | 549.2 |
| CU0719 | 549.4 |
| CU0800 | 550.2 |
| CU0574 | 550.5 |
| CU0827 | 550.5 |
| CU0554 | 551.0 |
| CU0605 | 551.1 |
| CU0631 | 551.1 |
| CU0717 | 551.1 |
| CU0517 | 551.4 |
| CU0544 | 551.4 |
| CU0594 | 551.5 |
| CU0511 | 552.5 |
| CU0630 | 553.0 |
| CU0531 | 553.1 |
| CU0716 | 553.3 |
| CU0570 | 553.4 |
| CU0556 | 553.5 |
| CU0635 | 553.5 |
| CU0564 | 554.0 |
| CU0530 | 554.2 |
| CU0669 | 554.2 |
| CU0647 | 554.6 |
| CU0660 | 555.0 |
| CU0794 | 555.0 |
| CU0742 | 555.1 |
| CU0560 | 555.3 |
| CU0538 | 555.4 |
| CU0821 | 555.4 |
| CU0615 | 555.5 |
| CU0600 | 556.2 |
| CU0569 | 556.5 |
| CU0553 | 556.9 |
| CU0765 | 558.2 |
| CU0527 | 559.4 |
| CU0731 | 560.1 |
| CU0724 | 560.2 |
| CU0814 | 560.3 |
| CU0811 | 562.0 |
| CU0696 | 562.2 |
| CU0676 | 563.1 |
| CU0739 | 563.1 |
| CU0565 | 563.2 |
| CU0571 | 563.2 |
| CU0788 | 563.2 |
| CU0746 | 563.5 |
| CU0732 | 564.9 |
| CU0723 | 565.0 |
| CU0779 | 565.1 |
| CU0643 | 565.2 |
| CU0562 | 565.5 |
| CU0582 | 566.2 |
| CU0589 | 566.3 |
| CU0598 | 566.3 |
| CU0515 | 567.3 |
| CU0524 | 567.3 |
| CU0572 | 567.4 |
| CU0516 | 568.2 |
| CU0532 | 568.2 |
| CU0591 | 568.2 |
| CU0693 | 568.2 |
| CU0706 | 568.3 |
| CU0529 | 568.4 |
| CU0610 | 568.4 |
| CU0603 | 568.9 |
| CU0836 | 569.0 |
| CU0838 | 569.0 |
| CU0541 | 569.4 |
| CU0792 | 570.1 |
| CU0547 | 570.2 |
| CU0595 | 570.2 |
| CU0518 | 570.4 |
| CU0540 | 570.4 |
| CU0641 | 570.8 |
| CU0622 | 571.1 |
| CU0509 | 571.3 |
| CU0640 | 571.8 |
| CU0599 | 572.0 |
| CU06I9 | 574.3 |
| CU0665 | 575.2 |
| CU0774 | 576.2 |
| CU0817 | 576.2 |
| CU0652 | 576.3 |
| CU0659 | 577.0 |
| CU0606 | 578.1 |
| CU0682 | 578.2 |
| CU0710 | 579.3 |
| CU0528 | 582.3 |
| CU0658 | 583.6 |
| CU0683 | 583.7 |
| CU0766 | 584.1 |
| CU0625 | 584.2 |
| CU0567 | 584.8 |
| CU0526 | 585.3 |
| CU0561 | 585.3 |
| CU0604 | 585.9 |
| CU0559 | 586.0 |
| CU0806 | 586.2 |
| CU0543 | 586.9 |
| CU0601 | 588.2 |
| CU0727 | 589.0 |
| CU0804 | 592.2 |
| CU0549 | 593.5 |
| CU0656 | 594.4 |
| CU0557 | 596.2 |
| CU0506 | 596.4 |
| CU0510 | 596.4 |
| CU0534 | 598.4 |
| CU0504 | 598.9 |
| CU0654 | 601.2 |
| CU0577 | 604.2 |
| CU0751 | 604.9 |
| CU0700 | 605.2 |
| CU0593 | 605.5 |
| CU0728 | 606.0 |
| CU0627 | 607.1 |
| CU0741 | 608.4 |
| CU0522 | 609.2 |
| CU0550 | 609.5 |
| CU0772 | 610.3 |
| CU0566 | 610.4 |
| CU0797 | 611.0 |
| CU0521 | 611.4 |
| CU0520 | 611.6 |
| CU0620 | 611.7 |
| CU0733 | 615.0 |
| CU0551 | 618.1 |
| CU0703 | 619.3 |
| CU0675 | 620.6 |
| CU0678 | 621.3 |
| CU0590 | 627.1 |
| CU0539 | 629.4 |
| CU0502 | 632.1 |
| CU0705 | 632.2 |
| CU0508 | 632.9 |
| CU0535 | 634.1 |
| CU0744 | 635.2 |
| CU0629 | 639.9 |
| CU0580 | 640.0 |
| CU0805 | 641.5 |
| CU0519 | 642.4 |
| CU0533 | 642.4 |
| CU0692 | 644.0 |
| CU0525 | 645.5 |
| CU0523 | 653.3 |
| CU0684 | 655.1 |
| CU0563 | 662.1 |

### Example 21. Kinetic Solubility

Compounds were analyzed for kinetic solubility values using a standard kinetic solubility assay format. First, test compounds were dissolved in DMSO (Aldrich Chemical Co., Milwaukee, Wis., USA) at a concentration of 0.010 mol/L and used to prepare standard calibrators in DMSO solution. 500.0 μM, 250.0 μM, 125.0 μM, 62.5 μM, 31.2 μM, and 15.6 μM standards were prepared in a Greiner UV-Star clear V-bottom 96-well plate (Greiner-Bio One, Germany) for generating a standard curve. Using Alfa Aesar's Sodium phosphate, 0.2 M buffer solution, pH 7.4 (Alfa Aesar, Haverhill, Mass., USA), aqueous phosphate buffered saline (PBS) was prepared by diluting with Milli-Q water for a final concentration of 0.05 M. The pH of the solution was adjusted to 7.4±0.1 using 0.1 M hydrochloric acid (Fisher Scientific Waltham, Mass., USA) or 0.1 M aqueous sodium hydroxide (Fisher Scientific Waltham, Mass., USA).

10 μL of 0.010 mol/L DMSO test compound stock solutions were pipetted to separate 1.5 mL centrifuge tubes in triplicate. To these tubes, 190 μl of 0.05 M PBS was added. Each tube was gently vortexed for 5 sec. The tubes were shaken for a minimum of 24 h at approximately 25° C. After shaking, the solutions were transferred to 0.5 mL centrifuge filter tubes (MilliporeSigma, Burlington, Mass., USA) and centrifuged at 10,000 rpm for 5 min. After centrifugation, supernatants were collected and used for compound concentration analysis.

Compound concentrations in each solution were determined using high pressure liquid chromatography (HPLC) with ultraviolet (UV) detection and comparison with external standards. For HPLC, a Waters Acquity CSH Phenyl-Hexyl column (2.1 mm×50 mm, 1.7 μm) was used together with a generic gradient method on a Waters H-class system equipped with a diode array detector: Solvent A=0.1% formic acid in water, Solvent B=0.1% formic acid in acetonitrile (MilliporeSigma, Burlington, Mass., USA), flow=0.65 mL/min, (t=0 min: 95% A-5% B, t=4.75 min: 0% A-100% B, t=4.85 min: 0% A-100% B, t=4.86 min: 95% A-5% B, t=5 min: 95% A-5% B). UV quantifications were performed at 270 nm. Solution concentrations were calculated using Microsoft Excel. For determination of kinetic solubility, each sample and standard were injected once using an injection volume of 1 μL. Resulting kinetic solubility values in 0.5 M PBS, pH 7.4, for each compound tested are presented in Table 27.

TABLE 27
Kinetic solubility

| | Kinetic |
|---|---|
| ID# | solubility (μM) |
| CU0643 | >500 |
| SC0183 | >500 |
| CU0257 | >500 |
| CU0196 | >500 |
| CU0109 | >500 |
| CU0108 | >500 |
| SC0172 | 490 |
| SC0146 | 486 |
| CU0523 | 486 |
| CU0175 | 486 |
| CU0562 | 480 |
| CU0136 | 479 |
| CU0224 | 466 |
| SC0204 | 465 |
| CU0258 | 462 |
| CU0550 | 455 |
| CU0106 | 453 |
| CU0644 | 450 |
| SC0163 | 450 |
| SC0007 | 445 |
| SC0178 | 443 |
| CU0672 | 440 |
| CU0260 | 436 |
| CU0255 | 436 |
| CU0170 | 435 |
| CU0675 | 430 |
| CU0105 | 427 |
| SC0164 | 426 |
| CU0178 | 423 |
| CU0187 | 417 |
| CU0207 | 415 |
| CU0521 | 413 |
| CU0160 | 413 |
| SC0184 | 411 |
| CU0140 | 404 |
| CU0123 | 404 |
| SC0105 | 399 |
| SC0017 | 398 |
| CU0671 | 397 |
| CU0201 | 389 |
| CU0721 | 378 |
| CU0247 | 376 |
| CU0173 | 368 |
| CU0520 | 366 |
| CU0141 | 366 |
| CU0623 | 364 |
| SC0171 | 356 |
| CU0177 | 351 |
| SC0156 | 348 |
| CU0186 | 348 |
| CU0579 | 339 |
| CU0239 | 333 |
| SC0154 | 315 |
| CU0511 | 310 |
| SC0177 | 309 |
| CU0110 | 295 |
| CU0533 | 294 |
| CU0246 | 287 |
| CU0226 | 286 |
| CU0145 | 278 |
| SC0137 | 275 |
| CU0620 | 275 |
| CU0558 | 271 |
| CU0104 | 269 |
| SC0134 | 266 |
| CU0107 | 266 |
| CU0543 | 256 |
| SC0114 | 252 |
| CU0564 | 249 |
| CU0214 | 209 |
| CU0244 | 200 |
| CU0501 | 195 |
| CU0613 | 190 |
| CU0618 | 179 |
| CU0611 | 170 |
| SC0009 | 167 |
| CU0245 | 142 |
| SC0108 | 141 |
| CU0667 | 139 |
| CU0190 | 139 |
| CU0589 | 131 |
| SC0106 | 119 |
| CU0211 | 112 |
| SC0002 | 111 |
| SC0008 | 103 |
| CU0614 | 101 |
| SC0120 | 96 |
| CU0534 | 85 |
| SC0122 | 81 |
| CU0161 | 77 |
| CU0752 | 75 |
| CU0188 | 73 |
| CU0677 | 71 |
| SC0001 | 66 |
| SC0119 | 65 |
| CU0231 | 64 |
| CU0503 | 62 |
| CU0541 | 61 |
| CU0505 | 60 |
| CU0154 | 58 |
| SC0107 | 52 |
| CU0212 | 52 |
| CU0544 | 50 |
| CU0528 | 50 |
| CU0529 | 50 |
| CU0518 | 47 |
| SC0011 | 43 |
| SC0174 | 40 |
| CU0610 | 37 |
| CU0560 | 31 |
| CU0261 | 31 |
| CU0638 | 30 |
| CU0516 | 30 |
| SC0103 | 24 |
| CU0256 | 22 |
| CU0563 | 19 |
| CU0213 | 17 |
| SC0158 | 16 |
| CU0577 | 13 |
| SC0112 | 13 |
| CU0551 | 12 |
| CU0553 | 12 |
| CU0673 | 11 |
| CU0500 | 10 |
| SC0060 | <10 |
| SC0167 | <10 |
| CU0559 | <10 |
| CU0556 | <10 |
| CU0570 | <10 |
| SC0162 | <10 |
| CU0522 | <10 |
| CU0513 | <10 |
| SC0127 | <10 |
| SC0115 | <10 |
| CU0595 | <10 |
| CU0567 | <10 |
| SC0116 | <10 |
| CU0540 | <10 |
| SC0128 | <10 |
| CU0598 | <10 |
| CU0526 | <10 |
| CU0546 | <10 |
| SC0104 | <10 |
| SC0148 | <10 |
| CU0515 | <10 |
| CU0524 | <10 |
| CU0243 | <10 |
| CU0232 | <10 |

### Example 22. Synthesis of Cyclic Urea Compounds and Intermediates

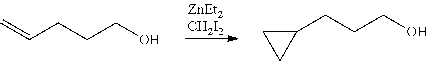

Diethylzinc solution (1.0 Min hexanes, 1.16 L, 1.16 mol) and trifluoroacetic acid (100 g, 0.87 mol) are added to a solution of 4-hexen-1-ol (50.0 g, 0.58 mol) mmol) in dichloromethane (1.5 L) at 0° C. The solution is stirred 0.5 h at 0° C. then diiodomethane (233 g, 0.87 mol) is added dropwise over 1 hour at this temperature. The resulting mixture is gradually warmed to room temperature and stirred overnight. The mixture is quenched with saturated aqueous ammonium chloride solution (800 mL) carefully, then filtered. The filtrate is separated, and the aqueous phase is extracted with dichloromethane (3×500 mL). The combined organic layers are washed with brine, dried and concentrated in vacuo to afford 3-cyclopropylpropan-1-ol.

The following compounds are prepared in a similar manner as described above.

| Alkene | Compound |
|---|---|
| 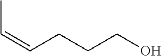 | 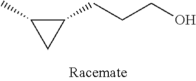 |
| 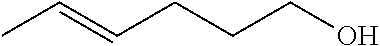 | 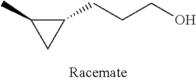 |
| 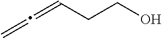 | 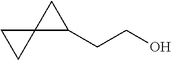 |

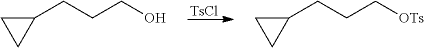 [SMILES: OCCCC1CC1 ; "TsCl" ; Cc1ccc(S(=O)(=O)OCCCC2CC2)cc1]

4-Dimethylaminopyridine (4.9 g, 40 mmol) is added to a solution of 3-cyclopropylpropan-1-ol (38.2 g, 0.38 mol) and triethylamine (78 g, 0.76 mol) in anhydrous dichloromethane (500 mL). The mixture is cooled to 0° C., then p-toluenesulfonyl chloride (86.6 g, 0.46 mol) is added portion-wise. The solution is gradually warmed to room temperature and stirred for 6 hours, then quenched with saturated aqueous sodium bicarbonate solution. The resulting mixture is extracted with dichloromethane. The organic layers are combined, washed with brine, dried and concentrated in vacuo. Purification by column chromatography over silica gel afforded 3-cyclopropylpropyl 4-methylbenzenesulfonate.

The following compounds are prepared in a similar manner as described above.

| Alcohol | Compound |
|---|---|
| 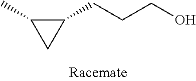 | 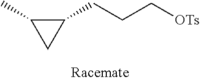 [SMILES: Cc1ccc(S(=O)(=O)OCCC[C@@H]2C[C@@H]2C)cc1] |
| 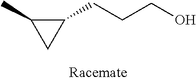 | 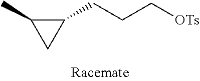 [SMILES: Cc1ccc(S(=O)(=O)OCCC[C@@H]2C[C@H]2C)cc1] |
| 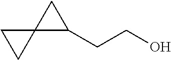 | 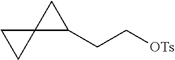 [SMILES: Cc1ccc(S(=O)(=O)OCCC2CC23CC3)cc1] |
| 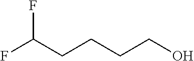 | 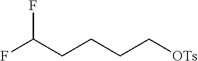 [SMILES: Cc1ccc(S(=O)(=O)OCCCCC(F)F)cc1] |
| 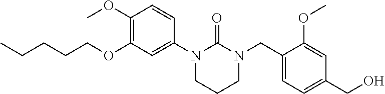 [SMILES: CCCCCOc1cc(N2CCCN(Cc3ccc(CO)cc3OC)C2=O)ccc1OC] | 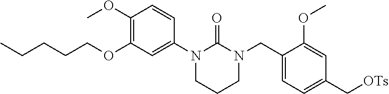 [SMILES: CCCCCOc1cc(N2CCCN(Cc3ccc(COS(=O)(=O)c4ccc(C)cc4)cc3OC)C2=O)ccc1OC] |
| 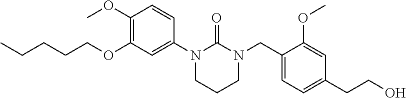 [SMILES: CCCCCOc1cc(N2CCCN(Cc3ccc(CCO)cc3OC)C2=O)ccc1OC] | 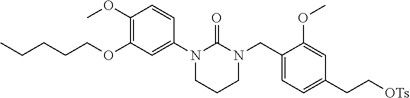 [SMILES: CCCCCOc1cc(N2CCCN(Cc3ccc(CCOS(=O)(=O)c4ccc(C)cc4)cc3OC)C2=O)ccc1OC] |
| 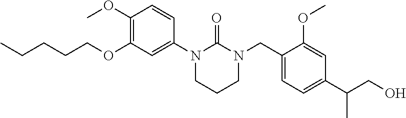 [SMILES: CCCCCOc1cc(N2CCCN(Cc3ccc(C(C)CO)cc3OC)C2=O)ccc1OC] | 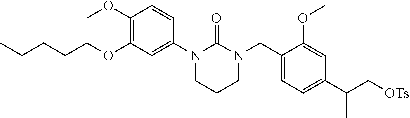 [SMILES: CCCCCOc1cc(N2CCCN(Cc3ccc(C(C)COS(=O)(=O)c4ccc(C)cc4)cc3OC)C2=O)ccc1OC] |
| 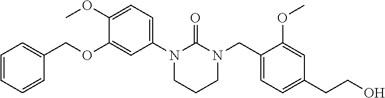 [SMILES: COc1cc(CCO)ccc1CN1CCCN(c2ccc(OC)c(OCc3ccccc3)c2)C1=O] | 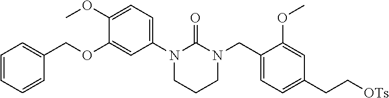 [SMILES: COc1cc(CCOS(=O)(=O)c2ccc(C)cc2)ccc1CN1CCCN(c2ccc(OC)c(OCc3ccccc3)c2)C1=O] |
| 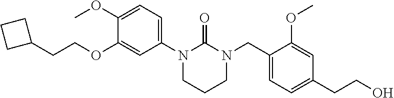 [SMILES: COc1cc(CCO)ccc1CN1CCCN(c2ccc(OC)c(OCCC3CCC3)c2)C1=O] | 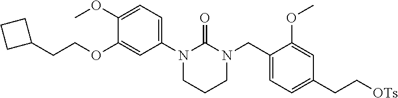 [SMILES: COc1cc(CCOS(=O)(=O)c2ccc(C)cc2)ccc1CN1CCCN(c2ccc(OC)c(OCCC3CCC3)c2)C1=O] |
| 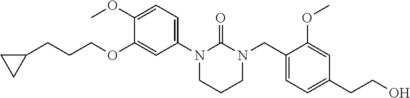 [SMILES: COc1cc(CCO)ccc1CN1CCCN(c2ccc(OC)c(OCCCC3CC3)c2)C1=O] | 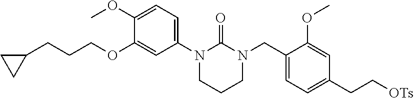 [SMILES: COc1cc(CCOS(=O)(=O)c2ccc(C)cc2)ccc1CN1CCCN(c2ccc(OC)c(OCCCC3CC3)c2)C1=O] |
| 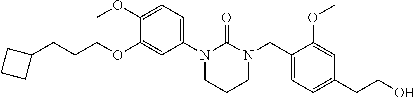 [SMILES: COc1cc(CCO)ccc1CN1CCCN(c2ccc(OC)c(OCCCC3CCC3)c2)C1=O] | 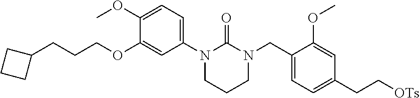 [SMILES: COc1cc(CCOS(=O)(=O)c2ccc(C)cc2)ccc1CN1CCCN(c2ccc(OC)c(OCCCC3CCC3)c2)C1=O] |
| 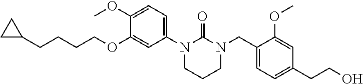 [SMILES: COc1cc(CCO)ccc1CN1CCCN(c2ccc(OC)c(OCCCCC3CC3)c2)C1=O] | 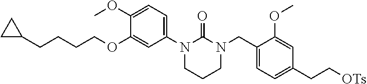 [SMILES: COc1cc(CCOS(=O)(=O)c2ccc(C)cc2)ccc1CN1CCCN(c2ccc(OC)c(OCCCCC3CC3)c2)C1=O] |
| 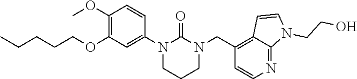 [SMILES: CCCCCOc1cc(N2CCCN(Cc3ccnc4c3ccn4CCO)C2=O)ccc1OC] | 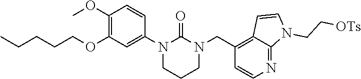 [SMILES: CCCCCOc1cc(N2CCCN(Cc3ccnc4c3ccn4CCOS(=O)(=O)c3ccc(C)cc3)C2=O)ccc1OC] |

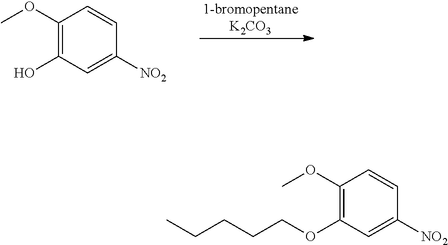 [SMILES: COc1ccc([N+](=O)[O-])cc1O ; CCCCCOc1cc([N+](=O)[O-])ccc1OC]

A solution of 2-methoxy-5-nitrophenol (100.0 g, 0.59 mol) in N,N-dimethylformamide (1.0 L) is treated with 1-bromopentane (117.2 g, 0.76 mol). Anhydrous potassium carbonate (122.5 g, 0.89 mol) is added at room temperature. The mixture is heated to 80° C. and stirred overnight at this temperature. The reaction mixture is cooled to room temperature, diluted with water (3 L), then extracted with ethyl acetate (3×2 L). The combined organic phases are washed with brine, dried over anhydrous sodium sulfate, then filtered and concentrated in vacuo to a volume of about 300 mL. The residue is diluted with hexane (1 L) and stirred for 10 minutes at room temperature to precipitate a white solid. The solids are collected by filtration and dried under vacuum to afford 1-methoxy-4-nitro-2-(pentyloxy)benzene.

The following compounds are prepared in a similar manner as described above.

| Phenol | Bromide | Compound |
|---|---|---|
| 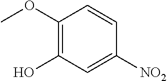 | 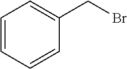 | 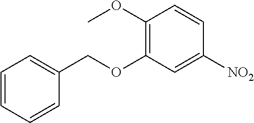 [SMILES: COc1ccc([N+](=O)[O-])cc1OCc1ccccc1] |
| 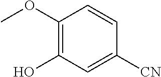 | 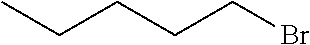 | 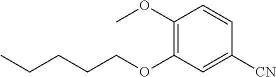 [SMILES: CCCCCOc1cc(C#N)ccc1OC] |
| 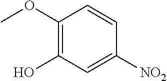 | 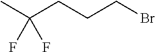 | 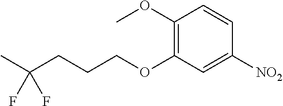 [SMILES: COc1ccc([N+](=O)[O-])cc1OCCCC(C)(F)F] |
| 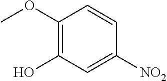 | 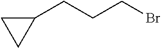 | 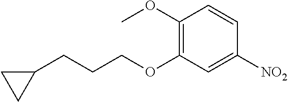 [SMILES: COc1ccc([N+](=O)[O-])cc1OCCCC1CC1] |
| 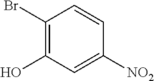 [SMILES: O=[N+]([O-])c1ccc(Br)c(O)c1] | 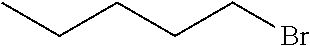 | 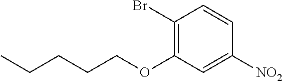 [SMILES: CCCCCOc1cc([N+](=O)[O-])ccc1Br] |
| 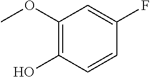 | 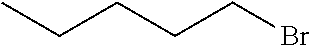 | 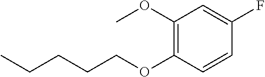 |
| 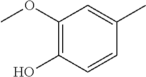 | 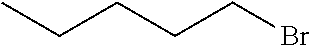 | 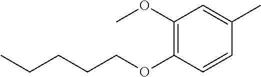 |
| 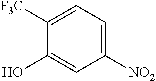 [SMILES: O=[N+]([O-])c1ccc(C(F)(F)F)c(O)c1] | 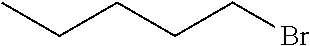 | 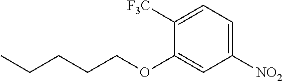 [SMILES: CCCCCOc1cc([N+](=O)[O-])ccc1C(F)(F)F] |
| 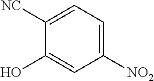 [SMILES: N#Cc1ccc([N+](=O)[O-])cc1O] | 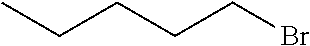 | 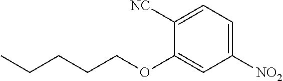 [SMILES: CCCCCOc1cc([N+](=O)[O-])ccc1C#N] |
| 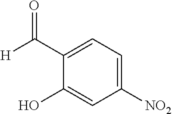 [SMILES: O=Cc1ccc([N+](=O)[O-])cc1O] | 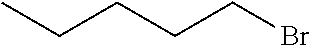 | 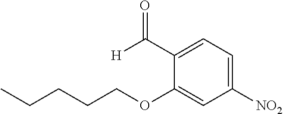 [SMILES: CCCCCOc1cc([N+](=O)[O-])ccc1C=O] |
| 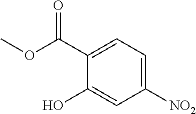 | 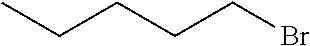 | 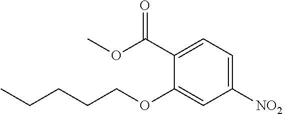 [SMILES: CCCCCOc1cc([N+](=O)[O-])ccc1C(=O)OC] |
| 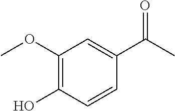 | 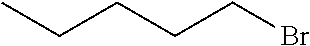 | 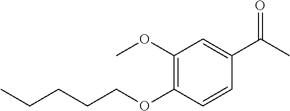 [SMILES: CCCCCOc1ccc(C(C)=O)cc1OC] |
| 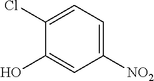 [SMILES: O=[N+]([O-])c1ccc(Cl)c(O)c1] | 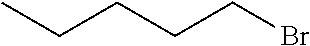 | 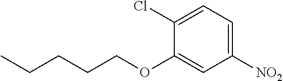 [SMILES: CCCCCOc1cc([N+](=O)[O-])ccc1Cl] |
| 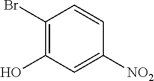 [SMILES: O=[N+]([O-])c1ccc(Br)c(O)c1] | 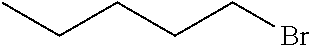 | 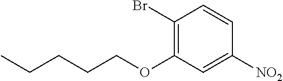 [SMILES: CCCCCOc1cc([N+](=O)[O-])ccc1Br] |
| 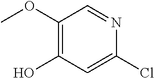 | 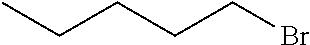 | 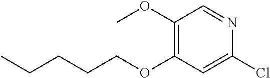 |
| 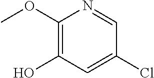 | 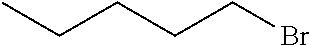 | 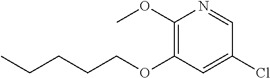 |
| 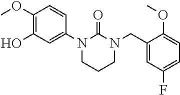 [SMILES: COc1ccc(N2CCCN(Cc3cc(F)ccc3OC)C2=O)cc1O] | 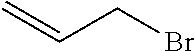 | 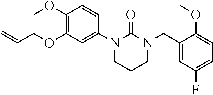 [SMILES: C=CCOc1cc(N2CCCN(Cc3cc(F)ccc3OC)C2=O)ccc1OC] |
| 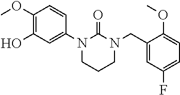 [SMILES: COc1ccc(N2CCCN(Cc3cc(F)ccc3OC)C2=O)cc1O] | 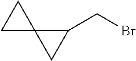 | 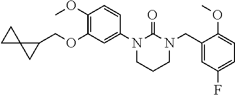 [SMILES: COc1ccc(F)cc1CN1CCCN(c2ccc(OC)c(OCC3CC34CC4)c2)C1=O] |
| 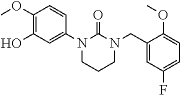 [SMILES: COc1ccc(N2CCCN(Cc3cc(F)ccc3OC)C2=O)cc1O] | 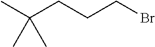 | 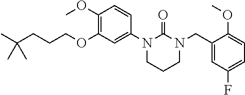 [SMILES: COc1ccc(F)cc1CN1CCCN(c2ccc(OC)c(OCCCC(C)(C)C)c2)C1=O] |
| 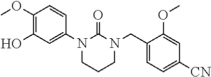 [SMILES: COc1ccc(N2CCCN(Cc3ccc(C#N)cc3OC)C2=O)cc1O] | 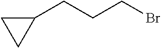 | 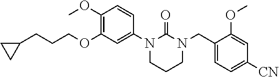 [SMILES: COc1cc(C#N)ccc1CN1CCCN(c2ccc(OC)c(OCCCC3CC3)c2)C1=O] |
| 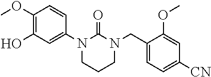 [SMILES: COc1ccc(N2CCCN(Cc3ccc(C#N)cc3OC)C2=O)cc1O] | 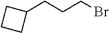 | 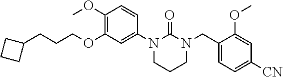 [SMILES: COc1cc(C#N)ccc1CN1CCCN(c2ccc(OC)c(OCCCC3CCC3)c2)C1=O] |
| 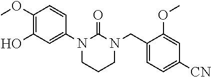 [SMILES: COc1ccc(N2CCCN(Cc3ccc(C#N)cc3OC)C2=O)cc1O] | 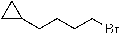 | 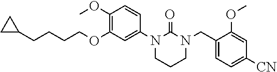 [SMILES: COc1cc(C#N)ccc1CN1CCCN(c2ccc(OC)c(OCCCCC3CC3)c2)C1=O] |
| 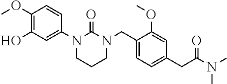 | 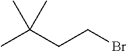 | 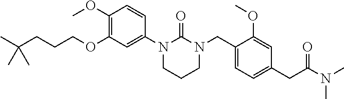 [SMILES: COc1cc(CC(=O)N(C)C)ccc1CN1CCCN(c2ccc(OC)c(OCCCC(C)(C)C)c2)C1=O] |
| 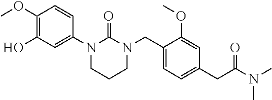 | 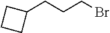 | 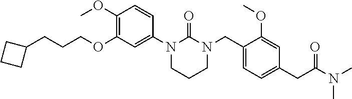 [SMILES: COc1cc(CC(=O)N(C)C)ccc1CN1CCCN(c2ccc(OC)c(OCCCC3CCC3)c2)C1=O] |
| 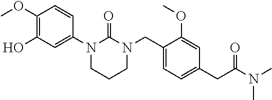 | 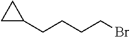 | 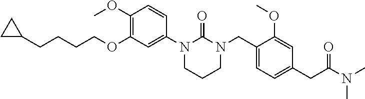 [SMILES: COc1cc(CC(=O)N(C)C)ccc1CN1CCCN(c2ccc(OC)c(OCCCCC3CC3)c2)C1=O] |
| 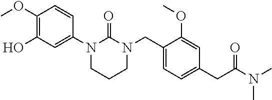 | 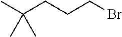 | 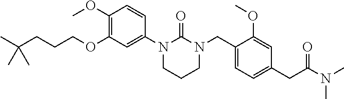 [SMILES: COc1cc(CC(=O)N(C)C)ccc1CN1CCCN(c2ccc(OC)c(OCCCC(C)(C)C)c2)C1=O] |
| 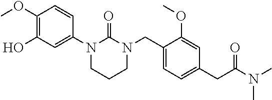 | 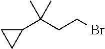 | 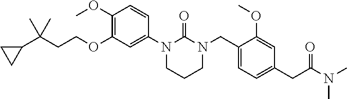 [SMILES: COc1cc(CC(=O)N(C)C)ccc1CN1CCCN(c2ccc(OC)c(OCCC(C)(C)C3CC3)c2)C1=O] |
| 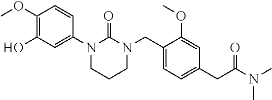 | 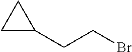 | 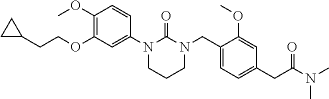 [SMILES: COc1cc(CC(=O)N(C)C)ccc1CN1CCCN(c2ccc(OC)c(OCCC3CC3)c2)C1=O] |
| 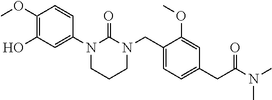 | 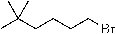 | 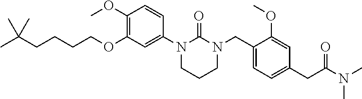 [SMILES: COc1cc(CC(=O)N(C)C)ccc1CN1CCCN(c2ccc(OC)c(OCCCCC(C)(C)C)c2)C1=O] |
| 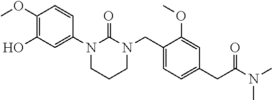 | 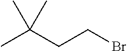 | 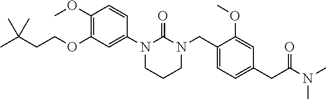 [SMILES: COc1cc(CC(=O)N(C)C)ccc1CN1CCCN(c2ccc(OC)c(OCCC(C)(C)C)c2)C1=O] |
| 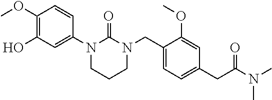 | 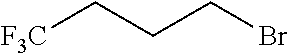 | 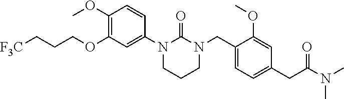 [SMILES: COc1cc(CC(=O)N(C)C)ccc1CN1CCCN(c2ccc(OC)c(OCCCC(F)(F)F)c2)C1=O] |
| 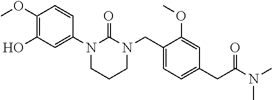 | 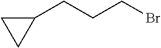 | 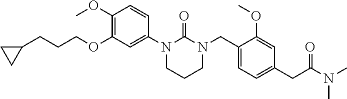 [SMILES: COc1cc(CC(=O)N(C)C)ccc1CN1CCCN(c2ccc(OC)c(OCCCC3CC3)c2)C1=O] |
| 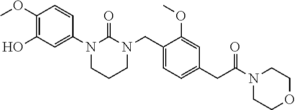 [SMILES: COc1ccc(N2CCCN(Cc3ccc(CC(=O)N4CCOCC4)cc3OC)C2=O)cc1O] | 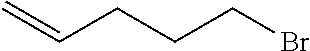 | 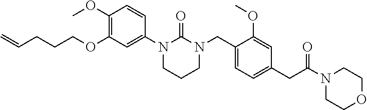 [SMILES: C=CCCCOc1cc(N2CCCN(Cc3ccc(CC(=O)N4CCOCC4)cc3OC)C2=O)ccc1OC] |
| 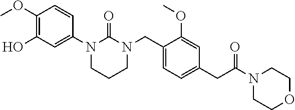 [SMILES: COc1ccc(N2CCCN(Cc3ccc(CC(=O)N4CCOCC4)cc3OC)C2=O)cc1O] | 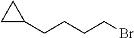 | 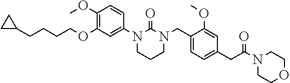 [SMILES: COc1cc(CC(=O)N2CCOCC2)ccc1CN1CCCN(c2ccc(OC)c(OCCCCC3CC3)c2)C1=O] |
| 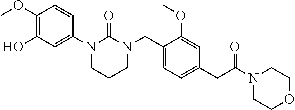 [SMILES: COc1ccc(N2CCCN(Cc3ccc(CC(=O)N4CCOCC4)cc3OC)C2=O)cc1O] | 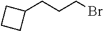 | 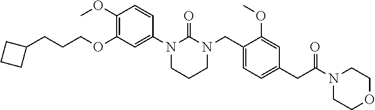 [SMILES: COc1cc(CC(=O)N2CCOCC2)ccc1CN1CCCN(c2ccc(OC)c(OCCCC3CCC3)c2)C1=O] |
| 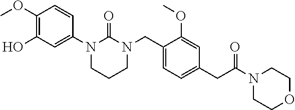 [SMILES: COc1ccc(N2CCCN(Cc3ccc(CC(=O)N4CCOCC4)cc3OC)C2=O)cc1O] | 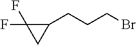 | 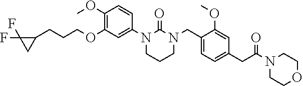 [SMILES: COc1cc(CC(=O)N2CCOCC2)ccc1CN1CCCN(c2ccc(OC)c(OCCCC3CC3(F)F)c2)C1=O] |
| 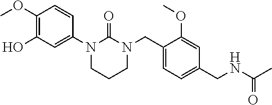 [SMILES: COc1ccc(N2CCCN(Cc3ccc(CNC(C)=O)cc3OC)C2=O)cc1O] | 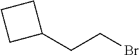 | 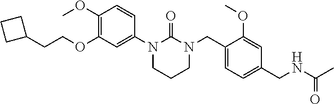 [SMILES: COc1cc(CNC(C)=O)ccc1CN1CCCN(c2ccc(OC)c(OCCC3CCC3)c2)C1=O] |
| 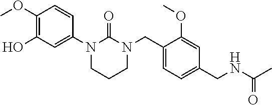 [SMILES: COc1ccc(N2CCCN(Cc3ccc(CNC(C)=O)cc3OC)C2=O)cc1O] | 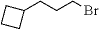 | 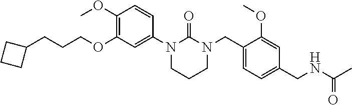 [SMILES: COc1cc(CNC(C)=O)ccc1CN1CCCN(c2ccc(OC)c(OCCCC3CCC3)c2)C1=O] |
| 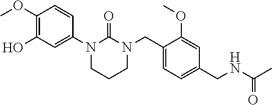 [SMILES: COc1ccc(N2CCCN(Cc3ccc(CNC(C)=O)cc3OC)C2=O)cc1O] | 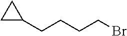 | 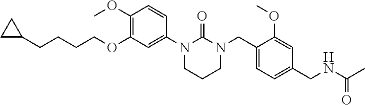 [SMILES: COc1cc(CNC(C)=O)ccc1CN1CCCN(c2ccc(OC)c(OCCCCC3CC3)c2)C1=O] |
| 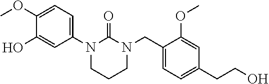 [SMILES: COc1ccc(N2CCCN(Cc3ccc(CCO)cc3OC)C2=O)cc1O] | 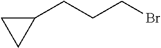 | 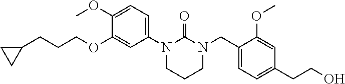 [SMILES: COc1cc(CCO)ccc1CN1CCCN(c2ccc(OC)c(OCCCC3CC3)c2)C1=O] |
| 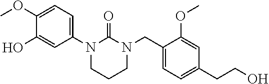 [SMILES: COc1ccc(N2CCCN(Cc3ccc(CCO)cc3OC)C2=O)cc1O] | 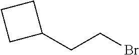 | 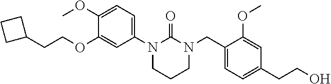 [SMILES: COc1cc(CCO)ccc1CN1CCCN(c2ccc(OC)c(OCCC3CCC3)c2)C1=O] |
| 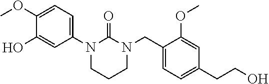 [SMILES: COc1ccc(N2CCCN(Cc3ccc(CCO)cc3OC)C2=O)cc1O] | 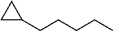 | 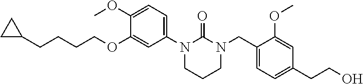 [SMILES: COc1cc(CCO)ccc1CN1CCCN(c2ccc(OC)c(OCCCCC3CC3)c2)C1=O] |
| 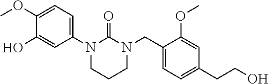 [SMILES: COc1ccc(N2CCCN(Cc3ccc(CCO)cc3OC)C2=O)cc1O] | 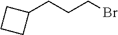 | 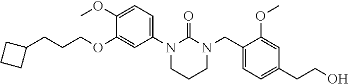 [SMILES: COc1cc(CCO)ccc1CN1CCCN(c2ccc(OC)c(OCCCC3CCC3)c2)C1=O] |
| 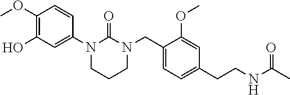 [SMILES: COc1ccc(N2CCCN(Cc3ccc(CCNC(C)=O)cc3OC)C2=O)cc1O] | 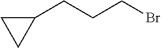 | 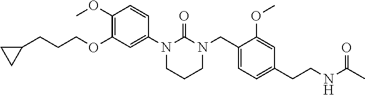 [SMILES: COc1cc(CCNC(C)=O)ccc1CN1CCCN(c2ccc(OC)c(OCCCC3CC3)c2)C1=O] |
| 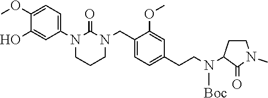 | 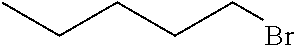 | 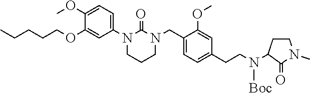 [SMILES: CCCCCOc1cc(N2CCCN(Cc3ccc(CCN(C(=O)OC(C)(C)C)C4CCN(C)C4=O)cc3OC)C2=O)ccc1OC] |
| 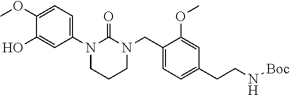 [SMILES: COc1ccc(N2CCCN(Cc3ccc(CCNC(=O)OC(C)(C)C)cc3OC)C2=O)cc1O] | 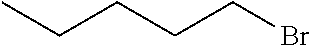 | 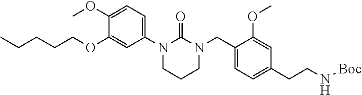 [SMILES: CCCCCOc1cc(N2CCCN(Cc3ccc(CCNC(=O)OC(C)(C)C)cc3OC)C2=O)ccc1OC] |
| 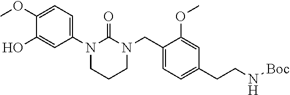 [SMILES: COc1ccc(N2CCCN(Cc3ccc(CCNC(=O)OC(C)(C)C)cc3OC)C2=O)cc1O] | 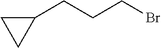 | 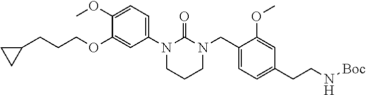 [SMILES: COc1cc(CCNC(=O)OC(C)(C)C)ccc1CN1CCCN(c2ccc(OC)c(OCCCC3CC3)c2)C1=O] |
| 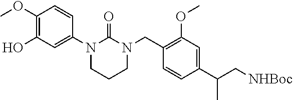 [SMILES: COc1ccc(N2CCCN(Cc3ccc(C(C)CNC(=O)OC(C)(C)C)cc3OC)C2=O)cc1O] | 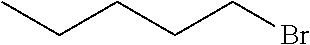 | 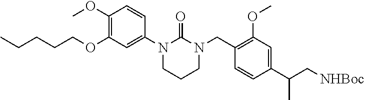 [SMILES: CCCCCOc1cc(N2CCCN(Cc3ccc(C(C)CNC(=O)OC(C)(C)C)cc3OC)C2=O)ccc1OC] |
| 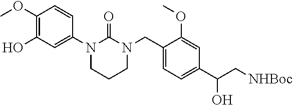 [SMILES: COc1ccc(N2CCCN(Cc3ccc(C(O)CNC(=O)OC(C)(C)C)cc3OC)C2=O)cc1O] | 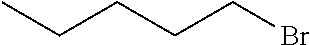 | 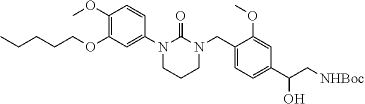 [SMILES: CCCCCOc1cc(N2CCCN(Cc3ccc(C(O)CNC(=O)OC(C)(C)C)cc3OC)C2=O)ccc1OC] |
| 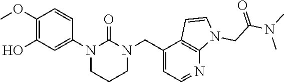 | 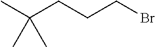 | 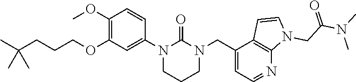 [SMILES: COc1ccc(N2CCCN(Cc3ccnc4c3ccn4CC(=O)N(C)C)C2=O)cc1OCCCC(C)(C)C] |
| 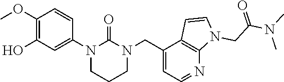 | 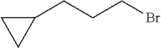 | 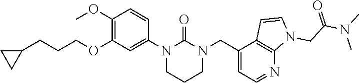 [SMILES: COc1ccc(N2CCCN(Cc3ccnc4c3ccn4CC(=O)N(C)C)C2=O)cc1OCCCC1CC1] |
| 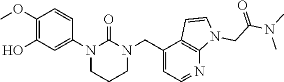 | 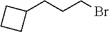 | 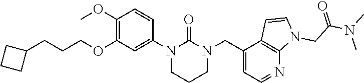 [SMILES: COc1ccc(N2CCCN(Cc3ccnc4c3ccn4CC(=O)N(C)C)C2=O)cc1OCCCC1CCC1] |
| 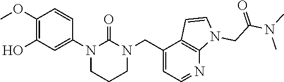 | 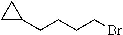 | 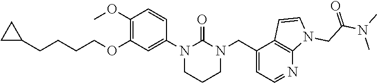 [SMILES: COc1ccc(N2CCCN(Cc3ccnc4c3ccn4CC(=O)N(C)C)C2=O)cc1OCCCCC1CC1] |
| 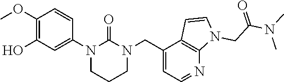 | 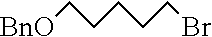 [SMILES: BrCCCCCOCc1ccccc1] | 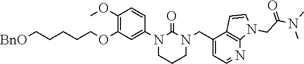 [SMILES: COc1ccc(N2CCCN(Cc3ccnc4c3ccn4CC(=O)N(C)C)C2=O)cc1OCCCCCOCc1ccccc1] |
| 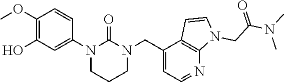 | 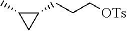 [SMILES: Cc1ccc(S(=O)(=O)OCCC[C@@H]2C[C@@H]2C)cc1] Racemate | 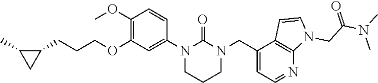 |
| | | Racemate |
| 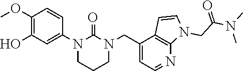 | 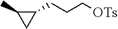 [SMILES: Cc1ccc(S(=O)(=O)OCCC[C@@H]2C[C@H]2C)cc1] Racemate | 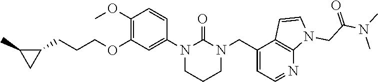 |
| | | Racemate |
| 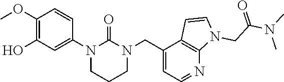 | 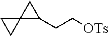 [SMILES: Cc1ccc(S(=O)(=O)OCCC2CC23CC3)cc1] | 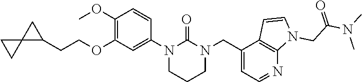 [SMILES: COc1ccc(N2CCCN(Cc3ccnc4c3ccn4CC(=O)N(C)C)C2=O)cc1OCCC1CC12CC2] |
| 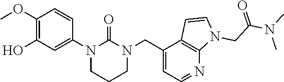 | 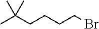 | 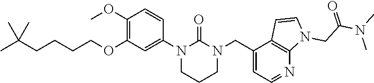 [SMILES: COc1ccc(N2CCCN(Cc3ccnc4c3ccn4CC(=O)N(C)C)C2=O)cc1OCCCCC(C)(C)C] |
| 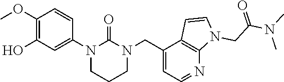 | 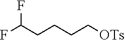 [SMILES: Cc1ccc(S(=O)(=O)OCCCCC(F)F)cc1] | 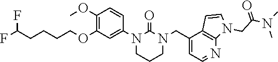 [SMILES: COc1ccc(N2CCCN(Cc3ccnc4c3ccn4CC(=O)N(C)C)C2=O)cc1OCCCCC(F)F] |
| 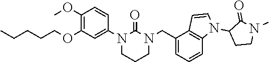 [SMILES: CCCCCOc1cc(N2CCCN(Cc3cccc4c3ccn4C3CCN(C)C3=O)C2=O)ccc1OC] | 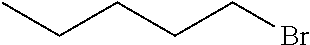 | 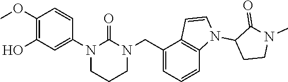 |
| 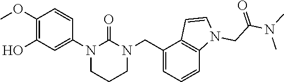 | 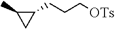 [SMILES: Cc1ccc(S(=O)(=O)OCCC[C@@H]2C[C@H]2C)cc1] Racemate | 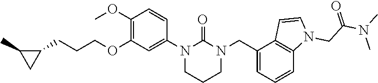 |
| | | Racemate |
| 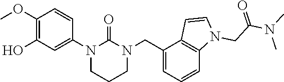 | 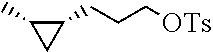 [SMILES: Cc1ccc(S(=O)(=O)OCCC[C@@H]2C[C@@H]2C)cc1] Racemate | 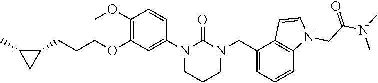 |
| | | Racemate |

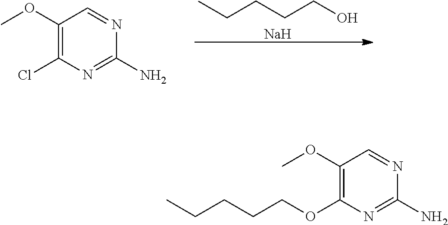

A solution of 1-pentanol (0.6 mL, 5.5 mmol) in anhydrous 1,4-dioxane (45 mL) is cooled to 0° C. then treated with sodium hydride (60 wt % in oil, 0.70 g, 17.5 mmol) added in portions. The reaction is stirred at 0° C. for 10 minutes. A solution of 4-chloro-5-methoxypyrimidin-2-amine (0.80 g, 5.0 mmol) in 1,4-dioxane (5 mL) is added to the reaction. Upon complete addition, the reaction is heated to 60° C. and stirred overnight at this temperature. The reaction is cooled to 0° C. and quenched with the addition of saturated aqueous ammonium chloride solution. The reaction is extracted with ethyl acetate. The combined organic layers are washed with brine, then dried over anhydrous sodium sulfate, filtered, and concentrated in vacuo. Purification by column chromatography over silica gel afforded 5-methoxy-4-(pentyloxy)pyrimidin-2-amine.

The following compounds are prepared in a similar manner as described above:

| Halide | Alcohol | Compound |
|---|---|---|
| 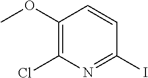 | 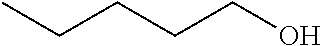 | 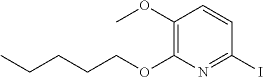 |

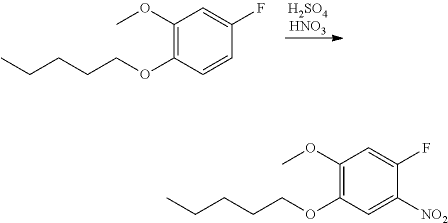 [SMILES: CCCCCOc1ccc(F)cc1OC ; CCCCCOc1cc([N+](=O)[O-])c(F)cc1OC]

Concentrated sulfuric acid (5 mL) is cooled to 0° C. before slowly adding 4-fluoro-2-methoxy-1-(pentyloxy)benzene (500 mg, 2.36 mmol) in portions. Concentrated nitric acid (1 mL) is added slowly dropwise at 0° C. The resulting mixture is stirred at 0° C. for 30 minutes then poured into ice and extracted with ethyl acetate. The combined organic layers are washed with saturated aqueous sodium bicarbonate, then dried over sodium sulfate, filtered, and concentrated in vacuo. Purification by column chromatography over silica gel afforded 1-fluoro-5-methoxy-2-nitro-4-(pentyloxy)benzene.

The following compounds are prepared in a similar manner as described above.

| Aromatic | Compound |
|---|---|
| 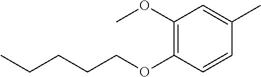 | 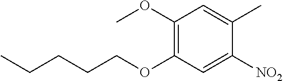 [SMILES: CCCCCOc1cc([N+](=O)[O-])c(C)cc1OC] |

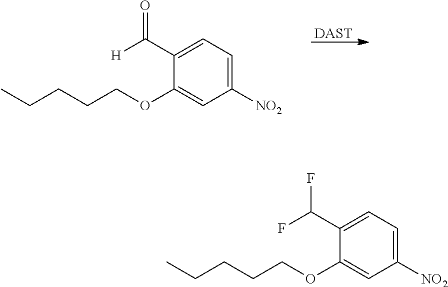 [SMILES: CCCCCOc1cc([N+](=O)[O-])ccc1C=O ; "DAST" ; CCCCCOc1cc([N+](=O)[O-])ccc1C(F)F]

(Diethylamino)sulfur trifluoride (DAST, 1.5 mL, 11.4 mmol) is cooled to 0° C. before adding 4-nitro-2-(pentyloxy)benzaldehyde (1.00 g, 4.21 mmol). The ice bath is removed, and the reaction is stirred at room temperature overnight. Ice-water is carefully added to quench the reaction. The mixture is extracted with ethyl acetate. The combined organic layers are washed with brine, then dried over anhydrous sodium sulfate, filtered, and concentrated in vacuo. Purification by column chromatography over silica gel afforded 1-(difluoromethyl)-4-nitro-2-(pentyloxy)benzene.

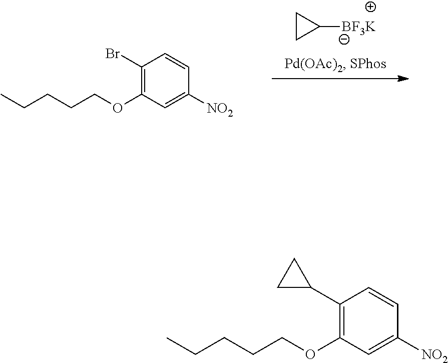

A solution of 1-bromo-4-nitro-2-(pentyloxy)benzene (100 mg, 0.26 mmol) in toluene (1.3 mL) and water (0.15 mL) is treated with 3.0 M aqueous potassium phosphate (0.26 mL, 0.77 mmol). The resulting mixture is sparged with argon for 10 minutes. To this mixture is added potassium cyclopropyltrifluoroborate (46 mg, 0.31 mmol), palladium diacetate (6 mg, 0.03 mmol), and 2-dicyclohexylphosphino-2′,6′-dimethoxy-1,1′-biphenyl (SPhos, 21 mg, 0.05 mmol). The resulting mixture is sparged with argon an additional 5 minutes. The reaction is sealed under an argon atmosphere then stirred at 100° C. overnight. The reaction is cooled to room temperature then diluted with water and ethyl acetate. The layers are separated, and the aqueous phase is extracted with ethyl acetate. The combined organic layers are dried over sodium sulfate, filtered, and concentrated in vacuo. Purification by column chromatography over reversed-phase C18 silica gel afforded 1-cyclopropyl-4-nitro-2-(pentyloxy)benzene.

The following compounds are prepared in a similar manner as described above.

| Bromide | Borate salt | Compound |
|---|---|---|
| 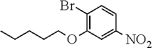 [SMILES: CCCCCOc1cc([N+](=O)[O-])ccc1Br] | 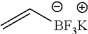 | 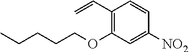 [SMILES: C=Cc1ccc([N+](=O)[O-])cc1OCCCCC] |
| 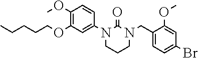 [SMILES: CCCCCOc1cc(N2CCCN(Cc3ccc(Br)cc3OC)C2=O)ccc1OC] | 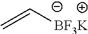 | 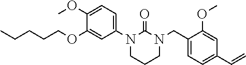 [SMILES: C=Cc1ccc(CN2CCCN(c3ccc(OC)c(OCCCCC)c3)C2=O)c(OC)c1] |
| 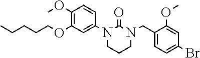 [SMILES: CCCCCOc1cc(N2CCCN(Cc3ccc(Br)cc3OC)C2=O)ccc1OC] | 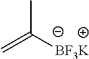 | 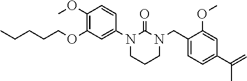 [SMILES: C=C(C)c1ccc(CN2CCCN(c3ccc(OC)c(OCCCCC)c3)C2=O)c(OC)c1] |
| 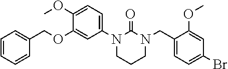 [SMILES: COc1cc(Br)ccc1CN1CCCN(c2ccc(OC)c(OCc3ccccc3)c2)C1=O] | 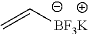 | 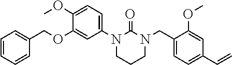 [SMILES: C=Cc1ccc(CN2CCCN(c3ccc(OC)c(OCc4ccccc4)c3)C2=O)c(OC)c1] |
| 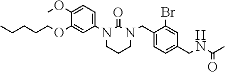 [SMILES: CCCCCOc1cc(N2CCCN(Cc3ccc(CNC(C)=O)cc3Br)C2=O)ccc1OC] | 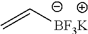 | 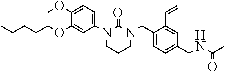 [SMILES: C=Cc1cc(CNC(C)=O)ccc1CN1CCCN(c2ccc(OC)c(OCCCCC)c2)C1=O] |
| 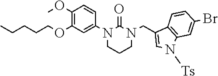 [SMILES: CCCCCOc1cc(N2CCCN(Cc3cn(S(=O)(=O)c4ccc(C)cc4)c4cc(Br)ccc34)C2=O)ccc1OC] | 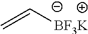 | 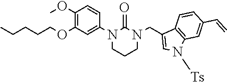 [SMILES: C=Cc1ccc2c(CN3CCCN(c4ccc(OC)c(OCCCCC)c4)C3=O)cn(S(=O)(=O)c3ccc(C)cc3)c2c1] |
| 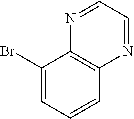 [SMILES: Brc1cccc2nccnc12] | 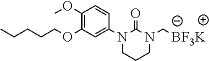 | 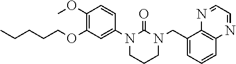 [SMILES: CCCCCOc1cc(N2CCCN(Cc3cccc4nccnc34)C2=O)ccc1OC] |
| 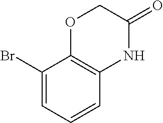 [SMILES: O=C1COc2c(Br)cccc2N1] | 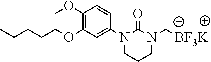 | 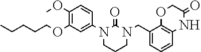 [SMILES: CCCCCOc1cc(N2CCCN(Cc3cccc4c3OCC(=O)N4)C2=O)ccc1OC] |
| 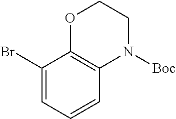 [SMILES: CC(C)(C)OC(=O)N1CCOc2c(Br)cccc21] | 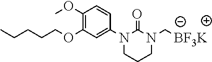 | 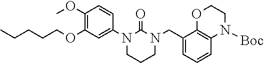 [SMILES: CCCCCOc1cc(N2CCCN(Cc3cccc4c3OCCN4C(=O)OC(C)(C)C)C2=O)ccc1OC] |
| 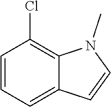 | 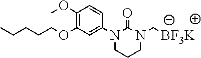 | 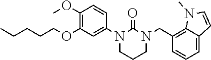 [SMILES: CCCCCOc1cc(N2CCCN(Cc3cccc4ccn(C)c34)C2=O)ccc1OC] |
| 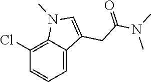 [SMILES: CN(C)C(=O)Cc1cn(C)c2c(Cl)cccc12] | 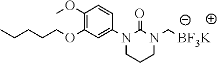 | 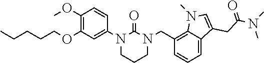 [SMILES: CCCCCOc1cc(N2CCCN(Cc3cccc4c(CC(=O)N(C)C)cn(C)c34)C2=O)ccc1OC] |

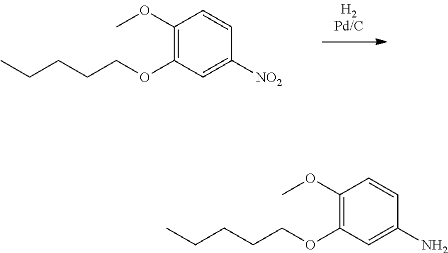 [SMILES: CCCCCOc1cc([N+](=O)[O-])ccc1OC ; CCCCCOc1cc(N)ccc1OC]

1-methoxy-4-nitro-2-(pentyloxy)benzene (119.5 g, 0.50 mol) is dissolved in methanol (1.5 L), and 10 wt % palladium on carbon (10 g) was added. The mixture is stirred overnight under an atmosphere of hydrogen. The reaction mixture is filtered through CELITE®, and the filter bed is washed with methanol. The filtered solution is concentrated in vacuo to afford 4-methoxy-3-(pentyloxy)aniline.

The following compounds are prepared in a similar manner as described above.

| Nitro | Compound |
|---|---|
| 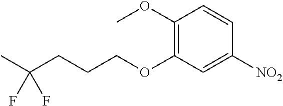 [SMILES: COc1ccc([N+](=O)[O-])cc1OCCCC(C)(F)F] | 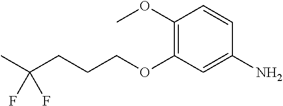 [SMILES: COc1ccc(N)cc1OCCCC(C)(F)F] |
| 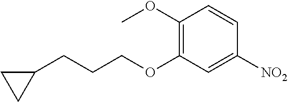 [SMILES: COc1ccc([N+](=O)[O-])cc1OCCCC1CC1] | 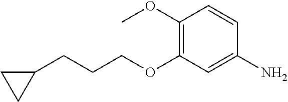 [SMILES: COc1ccc(N)cc1OCCCC1CC1] |
| 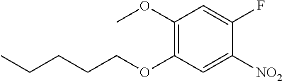 [SMILES: CCCCCOc1cc([N+](=O)[O-])c(F)cc1OC] | 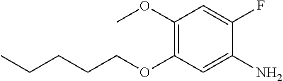 [SMILES: CCCCCOc1cc(N)c(F)cc1OC] |
| 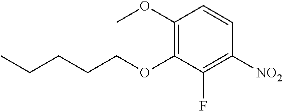 [SMILES: CCCCCOc1c(OC)ccc([N+](=O)[O-])c1F] | 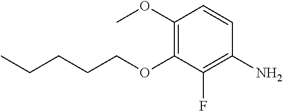 [SMILES: CCCCCOc1c(OC)ccc(N)c1F] |
| 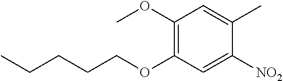 [SMILES: CCCCCOc1cc([N+](=O)[O-])c(C)cc1OC] | 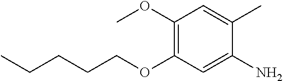 [SMILES: CCCCCOc1cc(N)c(C)cc1OC] |
| 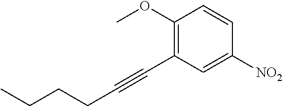 [SMILES: CCCCC#Cc1cc([N+](=O)[O-])ccc1OC] | 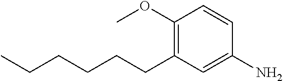 |
| 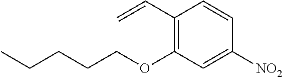 [SMILES: C=Cc1ccc([N+](=O)[O-])cc1OCCCCC] | 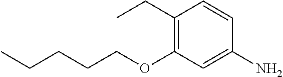 |
| 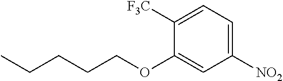 [SMILES: CCCCCOc1cc([N+](=O)[O-])ccc1C(F)(F)F] | 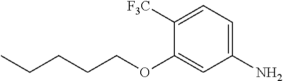 [SMILES: CCCCCOc1cc(N)ccc1C(F)(F)F] |
| 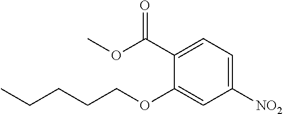 [SMILES: CCCCCOc1cc([N+](=O)[O-])ccc1C(=O)OC] | 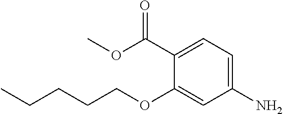 [SMILES: CCCCCOc1cc(N)ccc1C(=O)OC] |

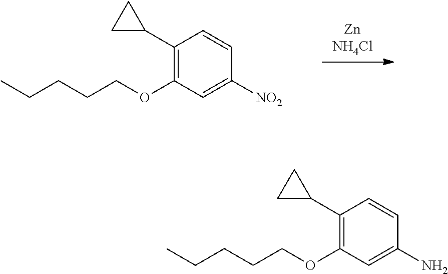 [SMILES: CCCCCOc1cc([N+](=O)[O-])ccc1C1CC1 ; CCCCCOc1cc(N)ccc1C1CC1]

A solution of 1-cyclopropyl-4-nitro-2-(pentyloxy)benzene (48 mg, 0.19 mmol) in acetone (0.6 mL) is treated with saturated aqueous ammonium chloride solution (275 μL, 1.93 mmol) and zinc powder (76 mg, 1.16 mmol). The resulting suspension is stirred at room temperature overnight. The reaction is diluted with acetone then filtered through CELITE®. The filter bed is washed with additional acetone. The filtered solution is concentrated in vacuo. This material is taken up in saturated aqueous sodium bicarbonate solution and extracted with dichloromethane. The combined organic layers are dried over anhydrous sodium sulfate, filtered, and concentrated in vacuo to afford compound 4-cyclopropyl-3-(pentyloxy)aniline.

The following compounds are prepared in a similar manner as described above.

| Nitro | Compound |
|---|---|
| 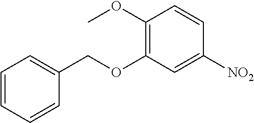 [SMILES: COc1ccc([N+](=O)[O-])cc1OCc1ccccc1] | 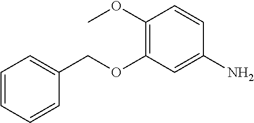 [SMILES: COc1ccc(N)cc1OCc1ccccc1] |
| 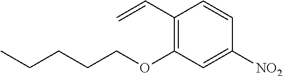 [SMILES: C=Cc1ccc([N+](=O)[O-])cc1OCCCCC] | 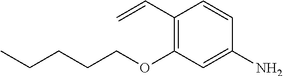 |
| 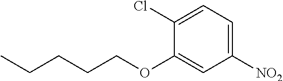 [SMILES: CCCCCOc1cc([N+](=O)[O-])ccc1Cl] | 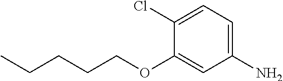 |
| 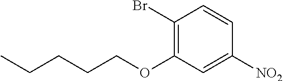 [SMILES: CCCCCOc1cc([N+](=O)[O-])ccc1Br] | 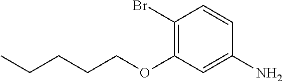 |
| 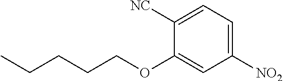 [SMILES: CCCCCOc1cc([N+](=O)[O-])ccc1C#N] | 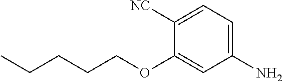 |
| 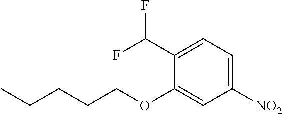 [SMILES: CCCCCOc1cc([N+](=O)[O-])ccc1C(F)F] | 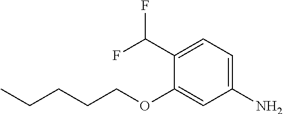 [SMILES: CCCCCOc1cc(N)ccc1C(F)F] |

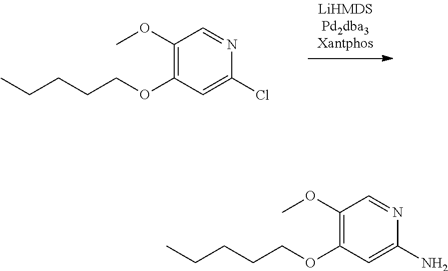

A solution of 2-chloro-5-methoxy-4-(pentyloxy)pyridine (2.82 g, 12.27 mmol), tris(dibenzylideneacetone)dipalladium(0) (0.56 g, 0.61 mmol), and 4,5-bis(diphenylphosphino)-9,9-dimethylxanthene (Xantphos, 0.71 g, 1.23 mmol) in anhydrous tetrahydrofuran (30 mL) is thoroughly sparged with nitrogen for several minutes at room temperature. Lithium bis(trimethylsilyl)amide (1.0M in tetrahydrofuran, 27 mL, 27.0 mmol) is added. The reaction is heated to 65° C. and stirred at this temperature for 5 hours. The reaction is quenched with 2N hydrochloric acid then washed with ethyl acetate. The organic extracts are discarded, and the remaining aqueous phase is treated with ION aqueous sodium hydroxide until pH ≈10-11. After a second extraction with ethyl acetate, the new organic extracts are dried over anhydrous magnesium sulfate, filtered, and concentrated in vacuo. Purification by column chromatography over silica gel afforded 5-methoxy-4-(pentyloxy)pyridin-2-amine.

The following compounds are prepared in a similar manner as described above.

| Halide | Compound |
|---|---|
| 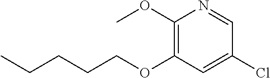 | 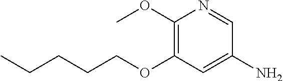 |
| 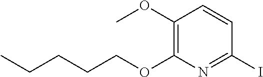 | 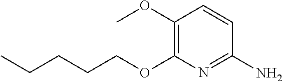 |

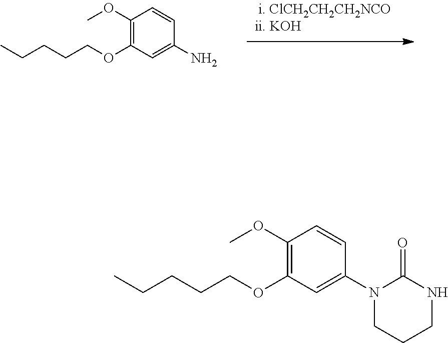 [SMILES: CCCCCOc1cc(N)ccc1OC ; CCCCCOc1cc(N2CCCNC2=O)ccc1OC]

To a solution of 4-methoxy-3-(pentyloxy)aniline (10.0 g, 47.8 mmol) in anhydrous tetrahydrofuran (100 mL) is added 3-chloropropyl isocyanate (5.6 g, 52.6 mmol) at room temperature. The mixture is stirred overnight at room temperature. Powdered potassium hydroxide (4.0 g, 71.8 mmol) is added, and the resulting mixture is stirred at 50° C. overnight. The reaction is diluted with water (300 mL), and the resulting precipitates are collected by filtration. The solids are washed with ethyl acetate and dried under vacuum to afford 1-(4-methoxy-3-(pentyloxy)phenyl)tetrahydropyrimidin-2(1H)-one.

The following compounds are prepared in a similar manner as described above.

| Amine | Isocyanate | Compound |
|---|---|---|
| 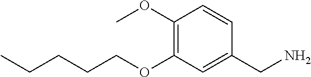 [SMILES: CCCCCOc1cc(CN)ccc1OC] | 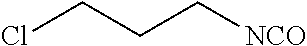 | 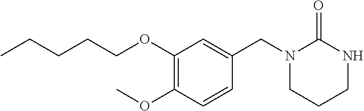 [SMILES: CCCCCOc1cc(CN2CCCNC2=O)ccc1OC] |
| 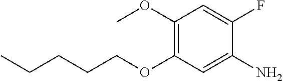 [SMILES: CCCCCOc1cc(N)c(F)cc1OC] | 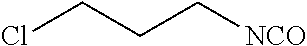 | 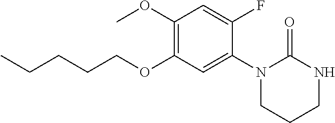 [SMILES: CCCCCOc1cc(N2CCCNC2=O)c(F)cc1OC] |
| 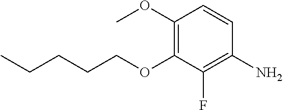 [SMILES: CCCCCOc1c(OC)ccc(N)c1F] | 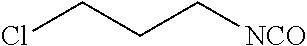 | 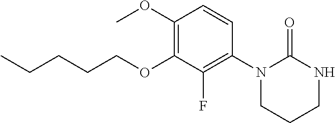 [SMILES: CCCCCOc1c(OC)ccc(N2CCCNC2=O)c1F] |
| 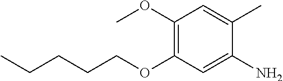 [SMILES: CCCCCOc1cc(N)c(C)cc1OC] | 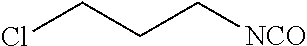 | 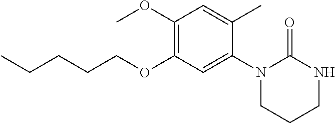 [SMILES: CCCCCOc1cc(N2CCCNC2=O)c(C)cc1OC] |
| 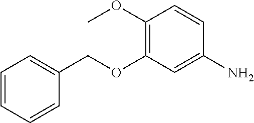 [SMILES: COc1ccc(N)cc1OCc1ccccc1] | 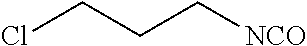 | 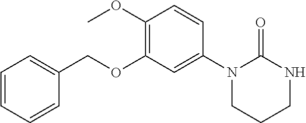 [SMILES: COc1ccc(N2CCCNC2=O)cc1OCc1ccccc1] |
| 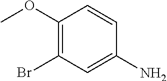 | 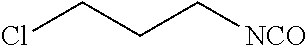 | 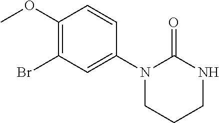 [SMILES: COc1ccc(N2CCCNC2=O)cc1Br] |
| 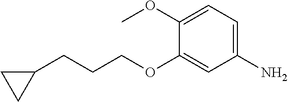 [SMILES: COc1ccc(N)cc1OCCCC1CC1] | 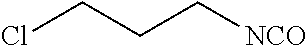 | 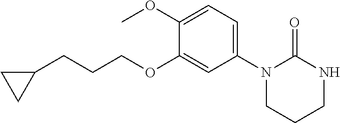 [SMILES: COc1ccc(N2CCCNC2=O)cc1OCCCC1CC1] |
| 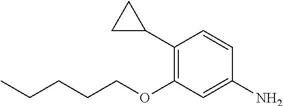 [SMILES: CCCCCOc1cc(N)ccc1C1CC1] | 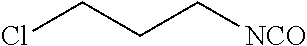 | 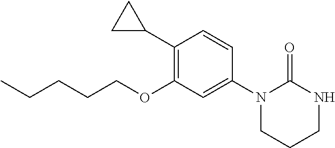 [SMILES: CCCCCOc1cc(N2CCCNC2=O)ccc1C1CC1] |
| 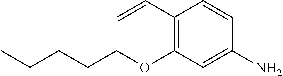 | 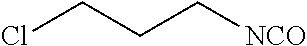 | 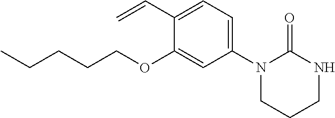 [SMILES: C=Cc1ccc(N2CCCNC2=O)cc1OCCCCC] |
| 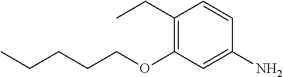 | 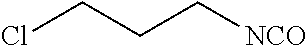 | 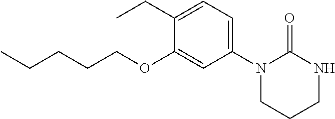 [SMILES: CCCCCOc1cc(N2CCCNC2=O)ccc1CC] |
| 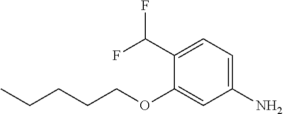 [SMILES: CCCCCOc1cc(N)ccc1C(F)F] | 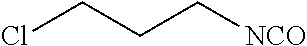 | 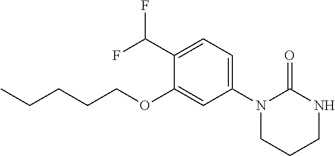 [SMILES: CCCCCOc1cc(N2CCCNC2=O)ccc1C(F)F] |
| 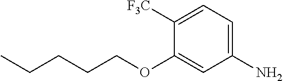 [SMILES: CCCCCOc1cc(N)ccc1C(F)(F)F] | 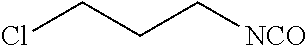 | 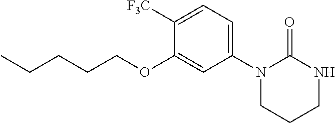 [SMILES: CCCCCOc1cc(N2CCCNC2=O)ccc1C(F)(F)F] |
| 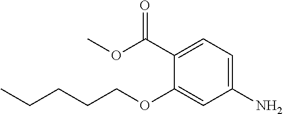 [SMILES: CCCCCOc1cc(N)ccc1C(=O)OC] | 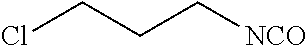 | 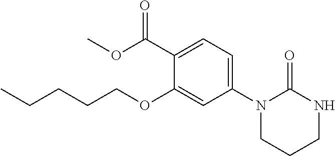 [SMILES: CCCCCOc1cc(N2CCCNC2=O)ccc1C(=O)OC] |
| 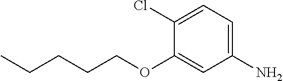 | 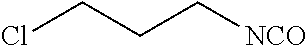 | 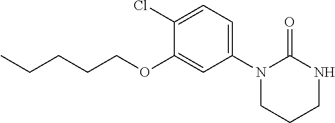 [SMILES: CCCCCOc1cc(N2CCCNC2=O)ccc1Cl] |
| 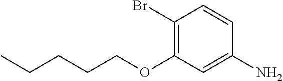 | 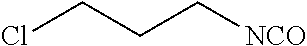 | 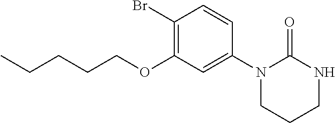 [SMILES: CCCCCOc1cc(N2CCCNC2=O)ccc1Br] |
| 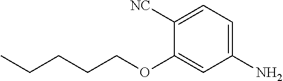 | 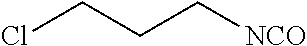 | 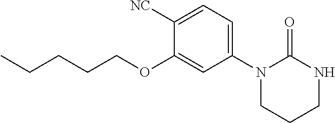 [SMILES: CCCCCOc1cc(N2CCCNC2=O)ccc1C#N] |
| 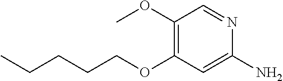 | 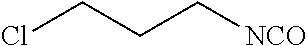 | 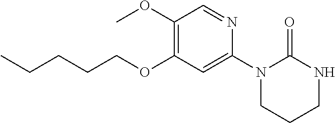 [SMILES: CCCCCOc1cc(N2CCCNC2=O)ncc1OC] |
| 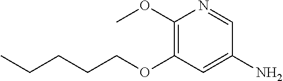 | 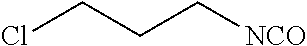 | 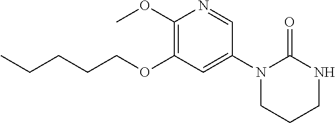 [SMILES: CCCCCOc1cc(N2CCCNC2=O)cnc1OC] |
| 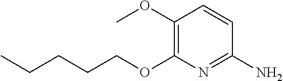 | 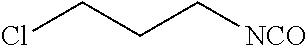 | 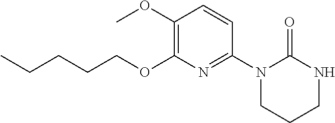 [SMILES: CCCCCOc1nc(N2CCCNC2=O)ccc1OC] |
| 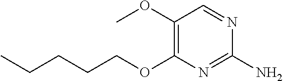 | 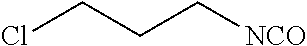 | 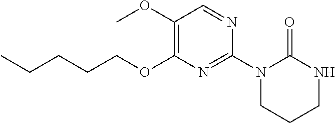 [SMILES: CCCCCOc1nc(N2CCCNC2=O)ncc1OC] |
| 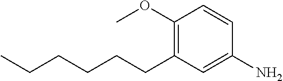 | 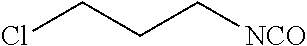 | 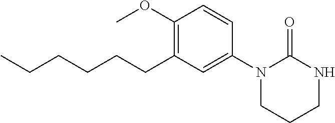 [SMILES: CCCCCCc1cc(N2CCCNC2=O)ccc1OC] |
| 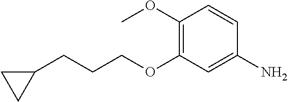 [SMILES: COc1ccc(N)cc1OCCCC1CC1] | 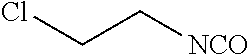 | 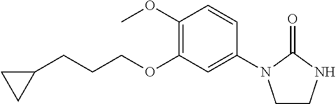 [SMILES: COc1ccc(N2CCNC2=O)cc1OCCCC1CC1] |

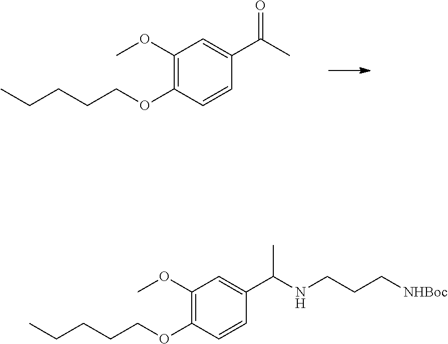

1-(3-methoxy-4-(pentyloxy)phenyl)ethan-1-one (100 mg, 0.42 mmol) and tert-butyl (3-aminopropyl)carbamate (147 mg, 0.84 mmol) are dissolved in methanol (4.2 mL) then cooled to 0′° C. Glacial acetic acid (3 uL) followed by sodium cyanoborohydride (40 mg, 0.63 mol) are added. The mixture is stirred for 1 hour at 0° C., and then gradually warmed to room temperature and stirred overnight. The mixture is concentrated in vacuo, and then quenched with water and extracted with dichloromethane. The combined organic layers are washed with brine, then dried over anhydrous sodium sulfate, filtered, and concentrated in vacuo. Purification by column chromatography over silica gel afforded tert-butyl (3-((1-(3-methoxy-4-(pentyloxy)phenyl)ethyl)amino)propyl)carbamate.

The following compounds are prepared in a similar manner as described above.

| Ketone/aldehyde | Amine | Compound |
|---|---|---|
| 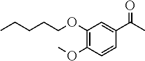 [SMILES: CCCCCOc1cc(C(C)=O)ccc1OC] | 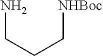 [SMILES: CC(C)(C)OC(=O)NCCCN] | 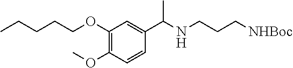 [SMILES: CCCCCOc1cc(C(C)NCCCNC(=O)OC(C)(C)C)ccc1OC] |
| 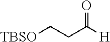 [SMILES: CC(C)(C)[Si](C)(C)OCCC=O] | 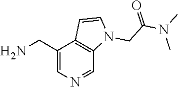 [SMILES: CN(C)C(=O)Cn1ccc2c(CN)cncc21] | 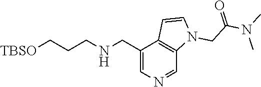 [SMILES: CN(C)C(=O)Cn1ccc2c(CNCCCO[Si](C)(C)C(C)(C)C)cncc21] |
| 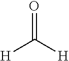 | 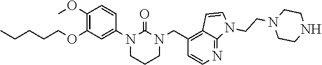 [SMILES: CCCCCOc1cc(N2CCCN(Cc3ccnc4c3ccn4CCN3CCNCC3)C2=O)ccc1OC] | 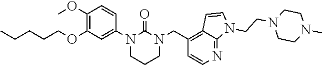 [SMILES: CCCCCOc1cc(N2CCCN(Cc3ccnc4c3ccn4CCN3CCN(C)CC3)C2=O)ccc1OC] |
| 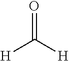 | 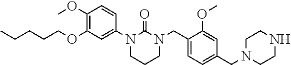 [SMILES: CCCCCOc1cc(N2CCCN(Cc3ccc(CN4CCNCC4)cc3OC)C2=O)ccc1OC] | 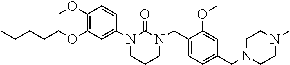 [SMILES: CCCCCOc1cc(N2CCCN(Cc3ccc(CN4CCN(C)CC4)cc3OC)C2=O)ccc1OC] |
| 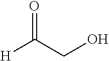 | 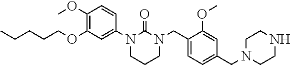 [SMILES: CCCCCOc1cc(N2CCCN(Cc3ccc(CN4CCNCC4)cc3OC)C2=O)ccc1OC] | 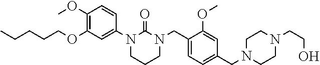 [SMILES: CCCCCOc1cc(N2CCCN(Cc3ccc(CN4CCN(CCO)CC4)cc3OC)C2=O)ccc1OC] |

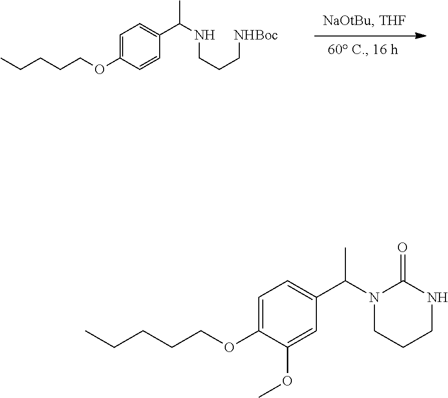 [SMILES: CCCCCOc1ccc(C(C)NCCCNC(=O)OC(C)(C)C)cc1 ; CCCCCOc1ccc(C(C)N2CCCNC2=O)cc1OC]

tert-butyl (3-((1-(3-methoxy-4-(pentyloxy)phenyl)ethyl)amino)propyl)carbamate (510 mg, 1.29 mmol) is dissolved with anhydrous tetrahydrofuran (3.9 mL) and treated with sodium tert butoxide (384 mg, 3.88 mmol). The resulting mixture is heated to 60° C. and stirred overnight at this temperature. After cooling to room temperature, water is added, then the reaction is extracted with ethyl acetate. The combined organic layers are dried over anhydrous sodium sulfate, filtered, and concentrated in vacuo to afford 1-(1-(3-methoxy-4-(pentyloxy)phenyl)ethyl)tetrahydropyrimidin-2(1H)-one.

The following compounds are prepared in a similar manner as described above.

| Protected amine | Compound |
|---|---|
| 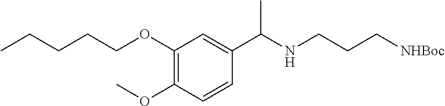 [SMILES: CCCCCOc1cc(C(C)NCCCNC(=O)OC(C)(C)C)ccc1OC] | 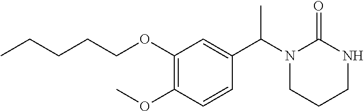 [SMILES: CCCCCOc1cc(C(C)N2CCCNC2=O)ccc1OC] |

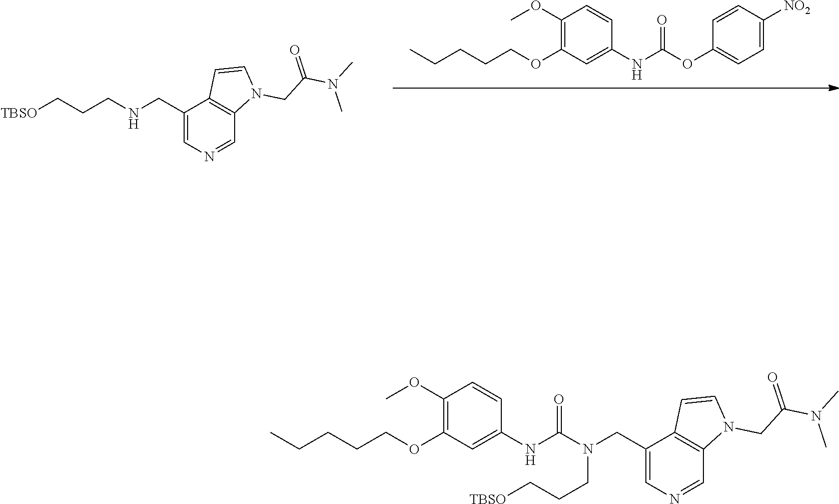 [SMILES: CN(C)C(=O)Cn1ccc2c(CNCCCO[Si](C)(C)C(C)(C)C)cncc21 ; CCCCCOc1cc(NC(=O)Oc2ccc([N+](=O)[O-])cc2)ccc1OC ; CCCCCOc1cc(NC(=O)N(CCCO[Si](C)(C)C(C)(C)C)Cc2cncc3c2ccn3CC(=O)N(C)C)ccc1OC]

A solution of 4-nitrophenyl (4-methoxy-3-(pentyloxy)phenyl)carbamate (225 mg, 0.60 mmol) and triethylamine (100 uL, 0.72 mmol) in anhydrous tetrahydrofuran (3 mL) is treated with a solution of 2-(4-(((3-((tert-butyldimethylsilyl)oxy)propyl)amino)methyl)-1H-pyrrolo[2,3-c]pyridin-1-yl)-N,N-dimethylacetamide (243 mg, 0.60 mmol) in anhydrous tetrahydrofuran (1 mL). Upon complete addition, the reaction is stirred at room temperature for 1 hour. Water is added, and the reaction mixture is extracted with ethyl acetate. The combined organic extracts are washed with brine, then dried over anhydrous sodium sulfate, filtered, and concentrated in vacuo. Purification by column chromatography over silica gel afforded 2-(4-((1-(3-((tert-butyldimethylsilyl)oxy)propyl)-3-(4-methoxy-3-(pentyloxy)phenyl)ureido)methyl)-1H-pyrrolo[2,3-c]pyridin-1-yl)-N,N-dimethylacetamide.

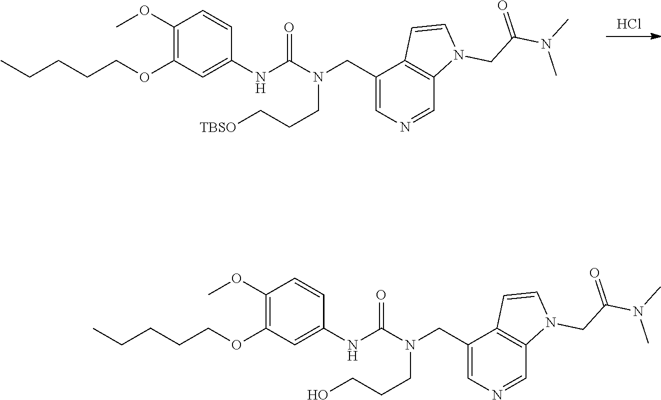

A solution of 2-(4-((1-(3-((tert-butyldimethylsilyl)oxy)propyl)-3-(4-methoxy-3-(pentyloxy)phenyl)ureido)methyl)-1H-pyrrolo[2,3-c]pyridin-1-yl)-N,N-dimethylacetamide (120 mg, 0.19 mmol) in tetrahydrofuran (2 mL) is cooled to 0° C. 1M hydrochloric acid (0.5 mL) is added, and the resulting mixture is stirred for 0.5 hours at 0° C. While still cold, the reaction is stopped with saturated aqueous sodium bicarbonate solution. The mixture is extracted with dichloromethane. The combined organic extracts are washed with saturated aqueous sodium bicarbonate solution, then dried over anhydrous sodium sulfate, filtered, and concentrated in vacuo. Purification by column chromatography over silica gel afforded 2-(4-((1-(3-hydroxypropyl)-3-(4-methoxy-3-(pentyloxy)phenyl)ureido)methyl)-1H-pyrrolo[2,3-c]pyridin-1-yl)-N,N-dimethylacetamide.

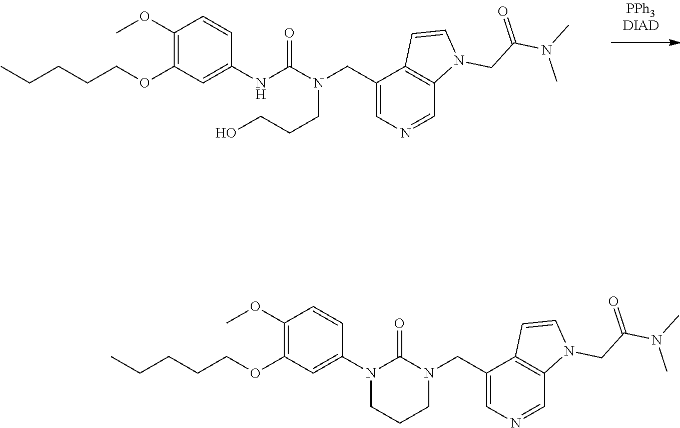

2-(4-((1-(3-hydroxypropyl)-3-(4-methoxy-3-(pentyloxy)phenyl)ureido)methyl 1H-pyrrolo[2,3-c]pyridin-1-yl)-N,N-dimethylacetamide (100 mg, 0.19 mmol)) and triphenylphosphine (60 mg g, 0.23 mmol) are dissolved in anhydrous tetrahydrofuran (2 mL) then cooled to 0° C. Diisopropyl azodicarboxylate (58 mg, 0.29 mmol) is added dropwise. The resulting solution is stirred for 1 hour at 0° C., and then allowed to warm to room temperature and stirred for another 16 hours. The reaction is quenched by adding water, and the resulting mixture is extracted with ethyl acetate. The combined organic phases are washed with brine, then dried over anhydrous sodium sulfate, filtered, and concentrated in vacuo. Purification by column chromatography over silica gel column afforded 2-(4-((3-(4-methoxy-3-(pentyloxy)phenyl)-2-oxotetrahydropyrimidin-1(2H)-yl)methyl)-1H-pyrrolo[2,3-c]pyridin-1-yl)-N,N-dimethylacetamide.

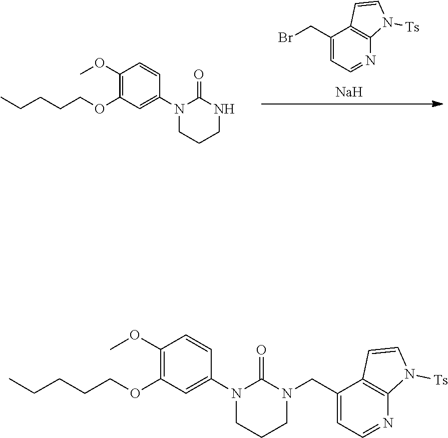

1-(4-methoxy-3-(pentyloxy)phenyl)tetrahydropyrimidin-2(1H)-one (22.0 g, 75 mmol) is suspended in anhydrous 2-methyltetrahydrofuran (660 mL) at 20° C. under nitrogen atmosphere. Sodium hydride (60% wt. in oil, 6.00 g, 151 mmol) is added in portions at 20° C. The reaction mixture is heated to 40° C. and stirred for 15 minutes at 40° C. A solution of (4-(bromomethyl)-1-tosyl-1H-pyrrolo[2,3-b]pyridine, 30 g, 79 mmol) in anhydrous 2-methyltetrahydrofuran (220 mL) is added over 1 hour. The reaction mixture is stirred at 40° C. for 20 hours. The reaction is poured into ice and extracted with ethyl acetate. The combined organic extracts are washed with brine then dried over sodium sulfate, filtered, and concentrated in vacuo to afford 1-(4-methoxy-3-(pentyloxy)phenyl)-3-((1-tosyl-1H-pyrrolo[2,3-b]pyridin-4-yl)methyl)tetrahydropyrimidin-2(1H)-one.

The following compounds are prepared in a similar manner as described above.

| Cyclic Urea | Bromide | Compound |
|---|---|---|
| 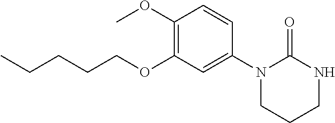 [SMILES: CCCCCOc1cc(N2CCCNC2=O)ccc1OC] | 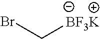 | 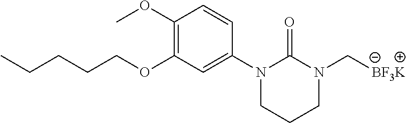 |
| 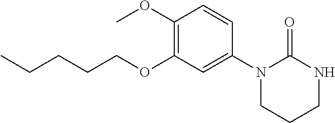 [SMILES: CCCCCOc1cc(N2CCCNC2=O)ccc1OC] | 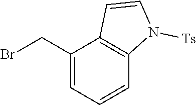 [SMILES: Cc1ccc(S(=O)(=O)n2ccc3c(CBr)cccc32)cc1] | 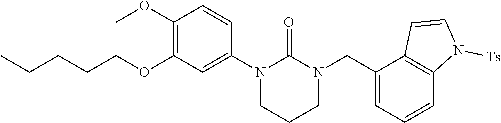 [SMILES: CCCCCOc1cc(N2CCCN(Cc3cccc4c3ccn4S(=O)(=O)c3ccc(C)cc3)C2=O)ccc1OC] |
| 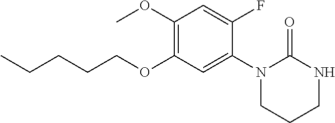 [SMILES: CCCCCOc1cc(N2CCCNC2=O)c(F)cc1OC] | 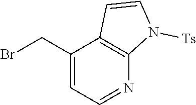 [SMILES: Cc1ccc(S(=O)(=O)n2ccc3c(CBr)ccnc32)cc1] | 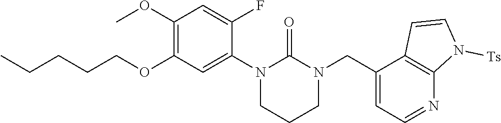 [SMILES: CCCCCOc1cc(N2CCCN(Cc3ccnc4c3ccn4S(=O)(=O)c3ccc(C)cc3)C2=O)c(F)cc1OC] |
| 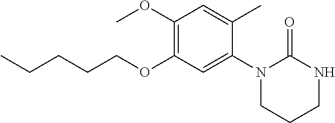 [SMILES: CCCCCOc1cc(N2CCCNC2=O)c(C)cc1OC] | 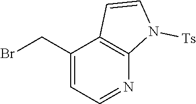 [SMILES: Cc1ccc(S(=O)(=O)n2ccc3c(CBr)ccnc32)cc1] | 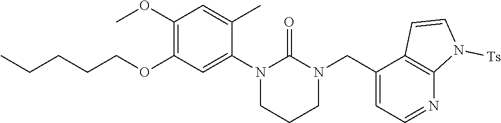 [SMILES: CCCCCOc1cc(N2CCCN(Cc3ccnc4c3ccn4S(=O)(=O)c3ccc(C)cc3)C2=O)c(C)cc1OC] |
| 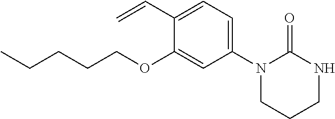 [SMILES: C=Cc1ccc(N2CCCNC2=O)cc1OCCCCC] | 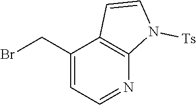 [SMILES: Cc1ccc(S(=O)(=O)n2ccc3c(CBr)ccnc32)cc1] | 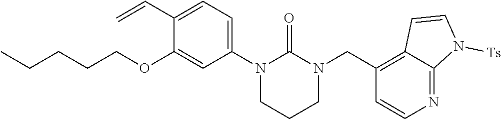 [SMILES: C=Cc1ccc(N2CCCN(Cc3ccnc4c3ccn4S(=O)(=O)c3ccc(C)cc3)C2=O)cc1OCCCCC] |
| 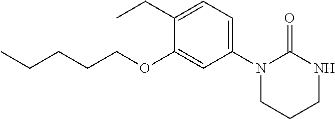 [SMILES: CCCCCOc1cc(N2CCCNC2=O)ccc1CC] | 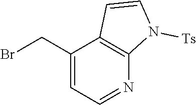 [SMILES: Cc1ccc(S(=O)(=O)n2ccc3c(CBr)ccnc32)cc1] | 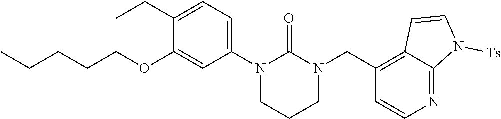 [SMILES: CCCCCOc1cc(N2CCCN(Cc3ccnc4c3ccn4S(=O)(=O)c3ccc(C)cc3)C2=O)ccc1CC] |
| 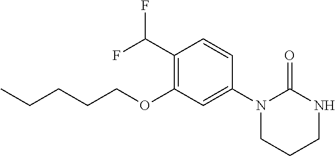 [SMILES: CCCCCOc1cc(N2CCCNC2=O)ccc1C(F)F] | 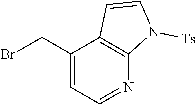 [SMILES: Cc1ccc(S(=O)(=O)n2ccc3c(CBr)ccnc32)cc1] | 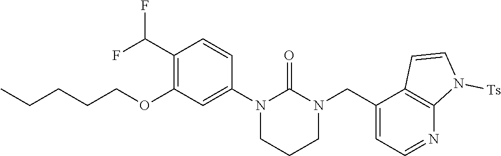 [SMILES: CCCCCOc1cc(N2CCCN(Cc3ccnc4c3ccn4S(=O)(=O)c3ccc(C)cc3)C2=O)ccc1C(F)F] |
| 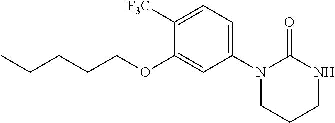 [SMILES: CCCCCOc1cc(N2CCCNC2=O)ccc1C(F)(F)F] | 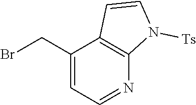 [SMILES: Cc1ccc(S(=O)(=O)n2ccc3c(CBr)ccnc32)cc1] | 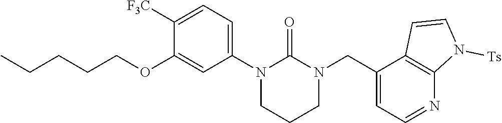 [SMILES: CCCCCOc1cc(N2CCCN(Cc3ccnc4c3ccn4S(=O)(=O)c3ccc(C)cc3)C2=O)ccc1C(F)(F)F] |
| 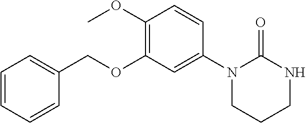 [SMILES: COc1ccc(N2CCCNC2=O)cc1OCc1ccccc1] | 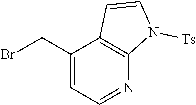 [SMILES: Cc1ccc(S(=O)(=O)n2ccc3c(CBr)ccnc32)cc1] | 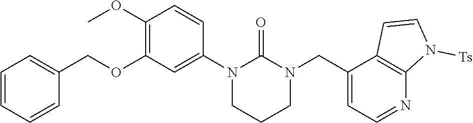 [SMILES: COc1ccc(N2CCCN(Cc3ccnc4c3ccn4S(=O)(=O)c3ccc(C)cc3)C2=O)cc1OCc1ccccc1] |
| 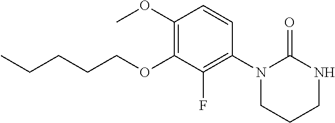 [SMILES: CCCCCOc1c(OC)ccc(N2CCCNC2=O)c1F] | 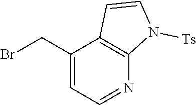 [SMILES: Cc1ccc(S(=O)(=O)n2ccc3c(CBr)ccnc32)cc1] | 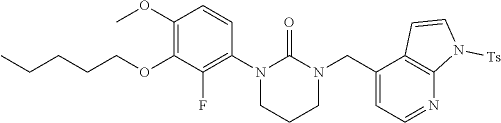 [SMILES: CCCCCOc1c(OC)ccc(N2CCCN(Cc3ccnc4c3ccn4S(=O)(=O)c3ccc(C)cc3)C2=O)c1F] |
| 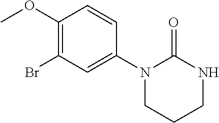 [SMILES: COc1ccc(N2CCCNC2=O)cc1Br] | 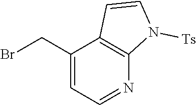 [SMILES: Cc1ccc(S(=O)(=O)n2ccc3c(CBr)ccnc32)cc1] | 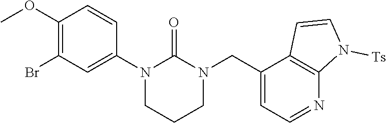 [SMILES: COc1ccc(N2CCCN(Cc3ccnc4c3ccn4S(=O)(=O)c3ccc(C)cc3)C2=O)cc1Br] |
| 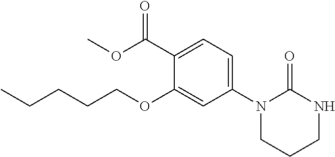 [SMILES: CCCCCOc1cc(N2CCCNC2=O)ccc1C(=O)OC] | 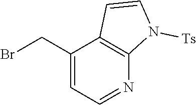 [SMILES: Cc1ccc(S(=O)(=O)n2ccc3c(CBr)ccnc32)cc1] | 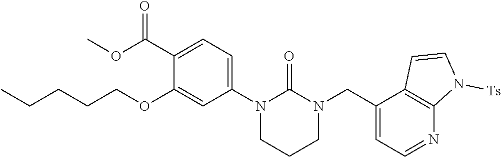 [SMILES: CCCCCOc1cc(N2CCCN(Cc3ccnc4c3ccn4S(=O)(=O)c3ccc(C)cc3)C2=O)ccc1C(=O)OC] |
| 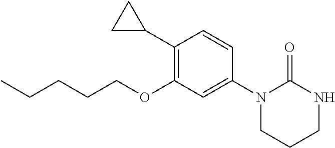 [SMILES: CCCCCOc1cc(N2CCCNC2=O)ccc1C1CC1] | 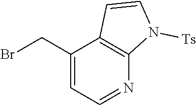 [SMILES: Cc1ccc(S(=O)(=O)n2ccc3c(CBr)ccnc32)cc1] | 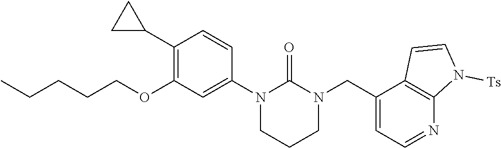 [SMILES: CCCCCOc1cc(N2CCCN(Cc3ccnc4c3ccn4S(=O)(=O)c3ccc(C)cc3)C2=O)ccc1C1CC1] |
| 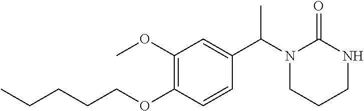 [SMILES: CCCCCOc1ccc(C(C)N2CCCNC2=O)cc1OC] | 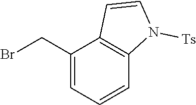 [SMILES: Cc1ccc(S(=O)(=O)n2ccc3c(CBr)cccc32)cc1] | 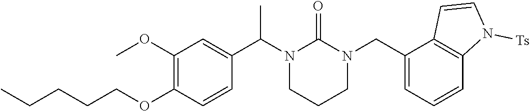 [SMILES: CCCCCOc1ccc(C(C)N2CCCN(Cc3cccc4c3ccn4S(=O)(=O)c3ccc(C)cc3)C2=O)cc1OC] |
| 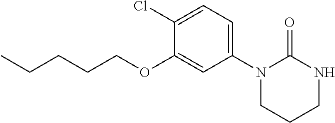 [SMILES: CCCCCOc1cc(N2CCCNC2=O)ccc1Cl] | 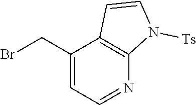 [SMILES: Cc1ccc(S(=O)(=O)n2ccc3c(CBr)ccnc32)cc1] | 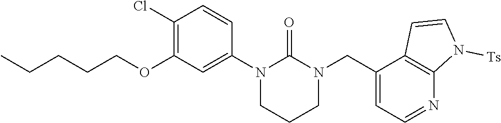 [SMILES: CCCCCOc1cc(N2CCCN(Cc3ccnc4c3ccn4S(=O)(=O)c3ccc(C)cc3)C2=O)ccc1Cl] |
| 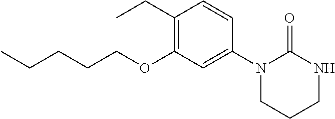 [SMILES: CCCCCOc1cc(N2CCCNC2=O)ccc1CC] | 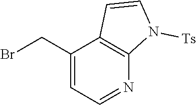 [SMILES: Cc1ccc(S(=O)(=O)n2ccc3c(CBr)ccnc32)cc1] | 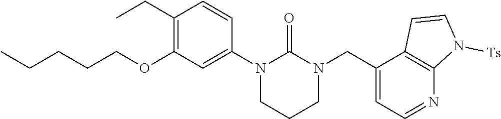 [SMILES: CCCCCOc1cc(N2CCCN(Cc3ccnc4c3ccn4S(=O)(=O)c3ccc(C)cc3)C2=O)ccc1CC] |
| 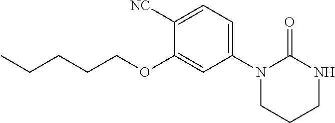 [SMILES: CCCCCOc1cc(N2CCCNC2=O)ccc1C#N] | 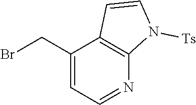 [SMILES: Cc1ccc(S(=O)(=O)n2ccc3c(CBr)ccnc32)cc1] | 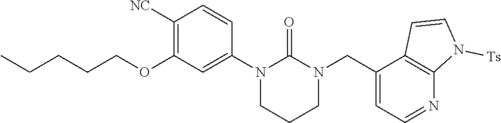 [SMILES: CCCCCOc1cc(N2CCCN(Cc3ccnc4c3ccn4S(=O)(=O)c3ccc(C)cc3)C2=O)ccc1C#N] |
| 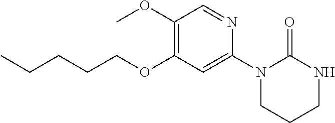 [SMILES: CCCCCOc1cc(N2CCCNC2=O)ncc1OC] | 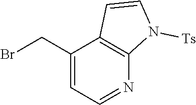 [SMILES: Cc1ccc(S(=O)(=O)n2ccc3c(CBr)ccnc32)cc1] | 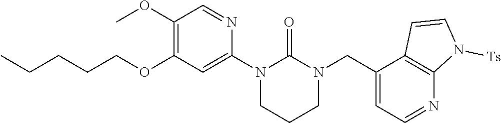 [SMILES: CCCCCOc1cc(N2CCCN(Cc3ccnc4c3ccn4S(=O)(=O)c3ccc(C)cc3)C2=O)ncc1OC] |
| 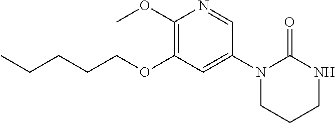 [SMILES: CCCCCOc1cc(N2CCCNC2=O)cnc1OC] | 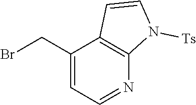 [SMILES: Cc1ccc(S(=O)(=O)n2ccc3c(CBr)ccnc32)cc1] | 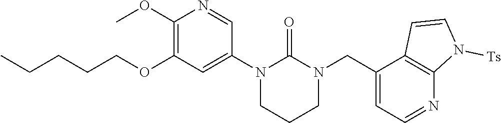 [SMILES: CCCCCOc1cc(N2CCCN(Cc3ccnc4c3ccn4S(=O)(=O)c3ccc(C)cc3)C2=O)cnc1OC] |
| 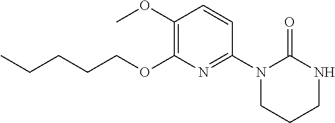 [SMILES: CCCCCOc1nc(N2CCCNC2=O)ccc1OC] | 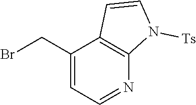 [SMILES: Cc1ccc(S(=O)(=O)n2ccc3c(CBr)ccnc32)cc1] | 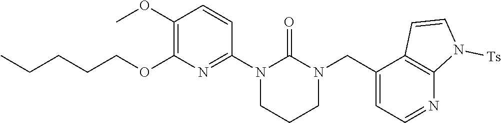 [SMILES: CCCCCOc1nc(N2CCCN(Cc3ccnc4c3ccn4S(=O)(=O)c3ccc(C)cc3)C2=O)ccc1OC] |
| 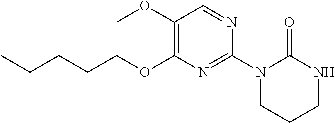 [SMILES: CCCCCOc1nc(N2CCCNC2=O)ncc1OC] | 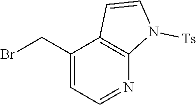 [SMILES: Cc1ccc(S(=O)(=O)n2ccc3c(CBr)ccnc32)cc1] | 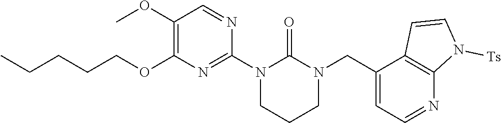 [SMILES: CCCCCOc1nc(N2CCCN(Cc3ccnc4c3ccn4S(=O)(=O)c3ccc(C)cc3)C2=O)ncc1OC] |
| 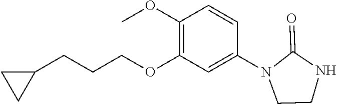 [SMILES: COc1ccc(N2CCNC2=O)cc1OCCCC1CC1] | 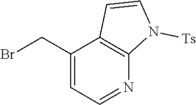 [SMILES: Cc1ccc(S(=O)(=O)n2ccc3c(CBr)ccnc32)cc1] | 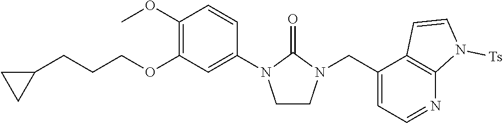 [SMILES: COc1ccc(N2CCN(Cc3ccnc4c3ccn4S(=O)(=O)c3ccc(C)cc3)C2=O)cc1OCCCC1CC1] |
| 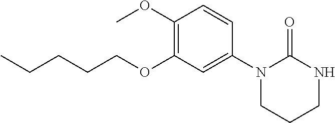 [SMILES: CCCCCOc1cc(N2CCCNC2=O)ccc1OC] | 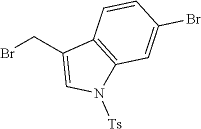 [SMILES: Cc1ccc(S(=O)(=O)n2cc(CBr)c3ccc(Br)cc32)cc1] | 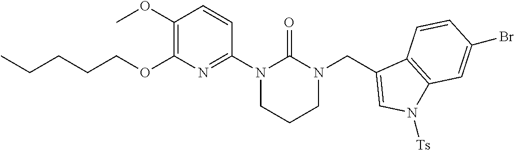 [SMILES: CCCCCOc1nc(N2CCCN(Cc3cn(S(=O)(=O)c4ccc(C)cc4)c4cc(Br)ccc34)C2=O)ccc1OC] |
| 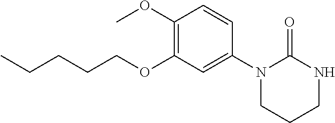 [SMILES: CCCCCOc1cc(N2CCCNC2=O)ccc1OC] | 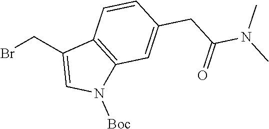 [SMILES: CN(C)C(=O)Cc1ccc2c(CBr)cn(C(=O)OC(C)(C)C)c2c1] | 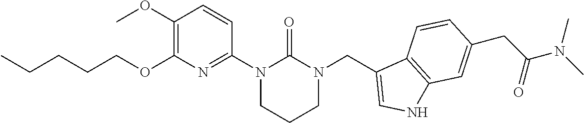 [SMILES: CCCCCOc1nc(N2CCCN(Cc3c[nH]c4cc(CC(=O)N(C)C)ccc34)C2=O)ccc1OC] |
| 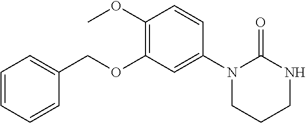 [SMILES: COc1ccc(N2CCCNC2=O)cc1OCc1ccccc1] | 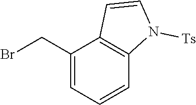 [SMILES: Cc1ccc(S(=O)(=O)n2ccc3c(CBr)cccc32)cc1] | 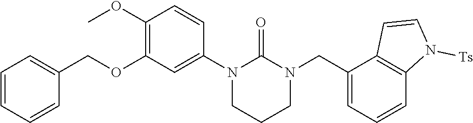 [SMILES: COc1ccc(N2CCCN(Cc3cccc4c3ccn4S(=O)(=O)c3ccc(C)cc3)C2=O)cc1OCc1ccccc1] |
| 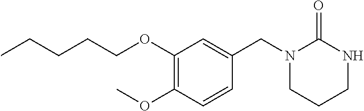 [SMILES: CCCCCOc1cc(CN2CCCNC2=O)ccc1OC] | 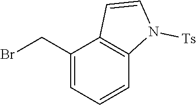 [SMILES: Cc1ccc(S(=O)(=O)n2ccc3c(CBr)cccc32)cc1] | 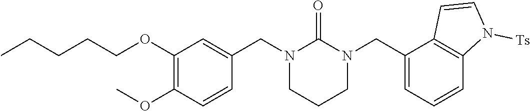 [SMILES: CCCCCOc1cc(CN2CCCN(Cc3cccc4c3ccn4S(=O)(=O)c3ccc(C)cc3)C2=O)ccc1OC] |
| 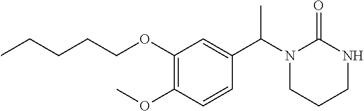 [SMILES: CCCCCOc1cc(C(C)N2CCCNC2=O)ccc1OC] | 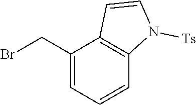 [SMILES: Cc1ccc(S(=O)(=O)n2ccc3c(CBr)cccc32)cc1] | 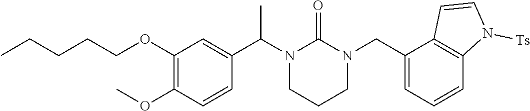 [SMILES: CCCCCOc1cc(C(C)N2CCCN(Cc3cccc4c3ccn4S(=O)(=O)c3ccc(C)cc3)C2=O)ccc1OC] |
| 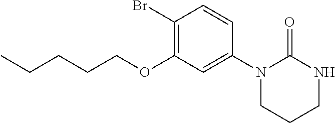 [SMILES: CCCCCOc1cc(N2CCCNC2=O)ccc1Br] | 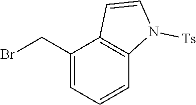 [SMILES: Cc1ccc(S(=O)(=O)n2ccc3c(CBr)cccc32)cc1] | 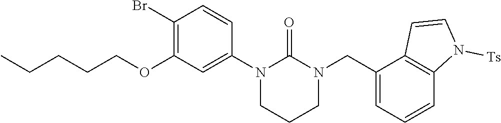 [SMILES: CCCCCOc1cc(N2CCCN(Cc3cccc4c3ccn4S(=O)(=O)c3ccc(C)cc3)C2=O)ccc1Br] |
| 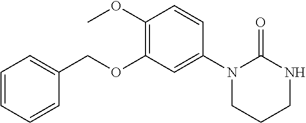 [SMILES: COc1ccc(N2CCCNC2=O)cc1OCc1ccccc1] | 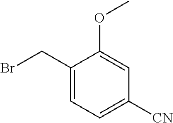 | 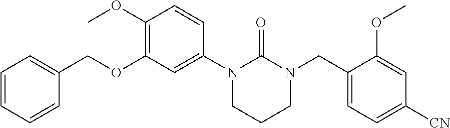 [SMILES: COc1cc(C#N)ccc1CN1CCCN(c2ccc(OC)c(OCc3ccccc3)c2)C1=O] |
| 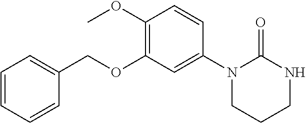 [SMILES: COc1ccc(N2CCCNC2=O)cc1OCc1ccccc1] | 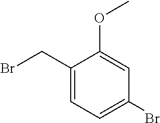 | 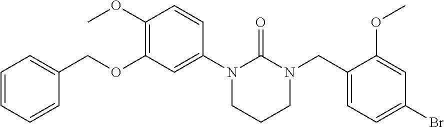 [SMILES: COc1cc(Br)ccc1CN1CCCN(c2ccc(OC)c(OCc3ccccc3)c2)C1=O] |
| 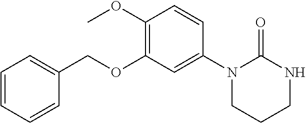 [SMILES: COc1ccc(N2CCCNC2=O)cc1OCc1ccccc1] | 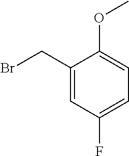 | 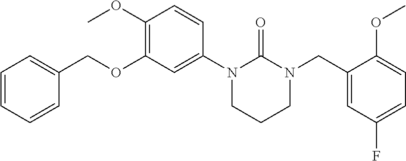 [SMILES: COc1ccc(F)cc1CN1CCCN(c2ccc(OC)c(OCc3ccccc3)c2)C1=O] |
| 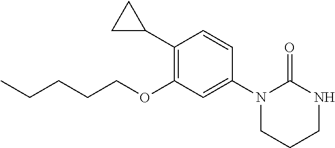 [SMILES: CCCCCOc1cc(N2CCCNC2=O)ccc1C1CC1] | 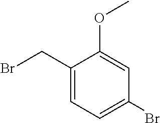 | 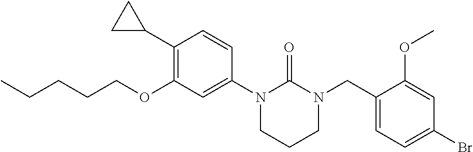 [SMILES: CCCCCOc1cc(N2CCCN(Cc3ccc(Br)cc3OC)C2=O)ccc1C1CC1] |
| 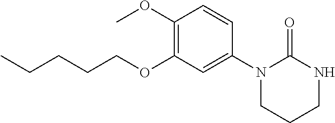 [SMILES: CCCCCOc1cc(N2CCCNC2=O)ccc1OC] | 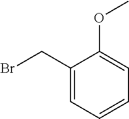 | 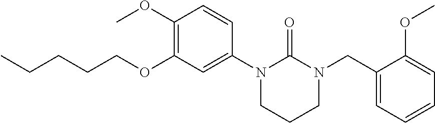 [SMILES: CCCCCOc1cc(N2CCCN(Cc3ccccc3OC)C2=O)ccc1OC] |
| 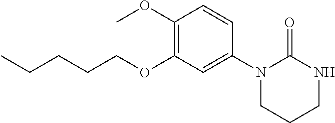 [SMILES: CCCCCOc1cc(N2CCCNC2=O)ccc1OC] | 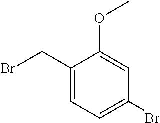 | 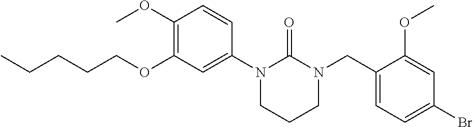 [SMILES: CCCCCOc1cc(N2CCCN(Cc3ccc(Br)cc3OC)C2=O)ccc1OC] |
| 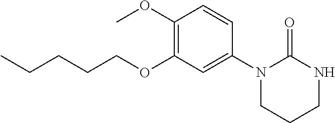 [SMILES: CCCCCOc1cc(N2CCCNC2=O)ccc1OC] | 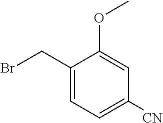 | 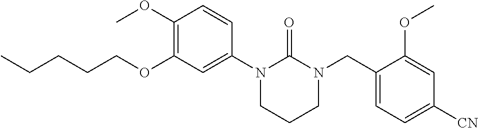 [SMILES: CCCCCOc1cc(N2CCCN(Cc3ccc(C#N)cc3OC)C2=O)ccc1OC] |
| 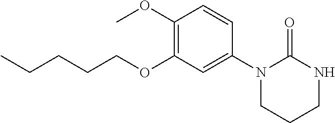 [SMILES: CCCCCOc1cc(N2CCCNC2=O)ccc1OC] | 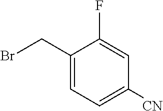 | 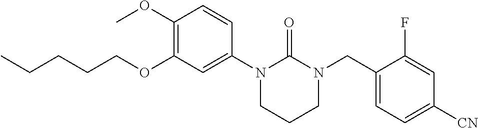 [SMILES: CCCCCOc1cc(N2CCCN(Cc3ccc(C#N)cc3F)C2=O)ccc1OC] |
| 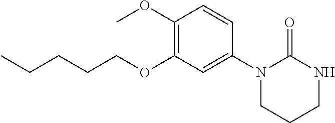 [SMILES: CCCCCOc1cc(N2CCCNC2=O)ccc1OC] | 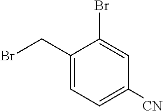 | 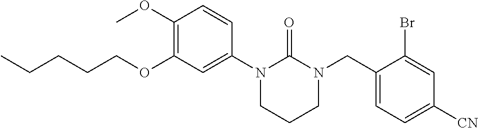 [SMILES: CCCCCOc1cc(N2CCCN(Cc3ccc(C#N)cc3Br)C2=O)ccc1OC] |
| 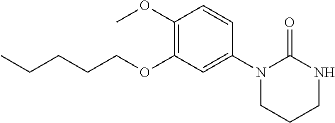 [SMILES: CCCCCOc1cc(N2CCCNC2=O)ccc1OC] | 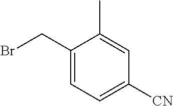 | 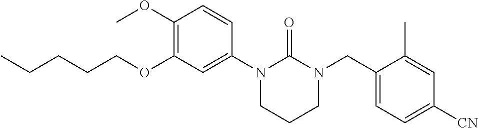 [SMILES: CCCCCOc1cc(N2CCCN(Cc3ccc(C#N)cc3C)C2=O)ccc1OC] |
| 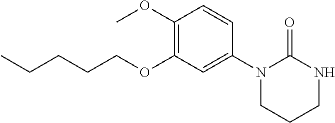 [SMILES: CCCCCOc1cc(N2CCCNC2=O)ccc1OC] | 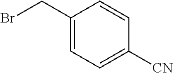 | 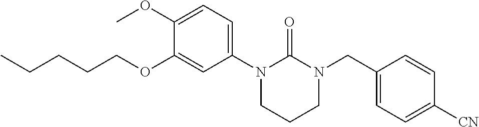 [SMILES: CCCCCOc1cc(N2CCCN(Cc3ccc(C#N)cc3)C2=O)ccc1OC] |
| 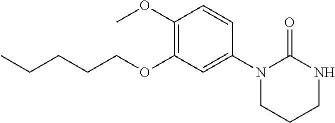 [SMILES: CCCCCOc1cc(N2CCCNC2=O)ccc1OC] | 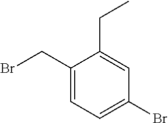 | 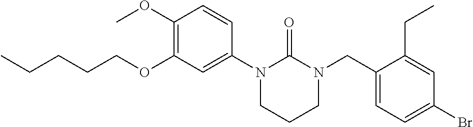 [SMILES: CCCCCOc1cc(N2CCCN(Cc3ccc(Br)cc3CC)C2=O)ccc1OC] |
| 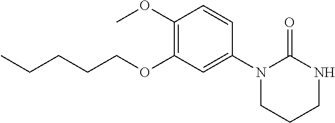 [SMILES: CCCCCOc1cc(N2CCCNC2=O)ccc1OC] | 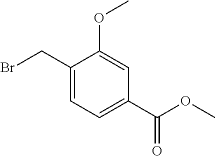 | 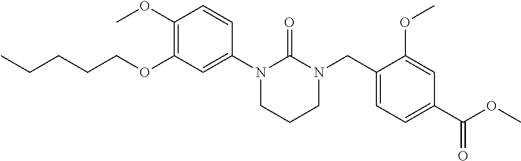 [SMILES: CCCCCOc1cc(N2CCCN(Cc3ccc(C(=O)OC)cc3OC)C2=O)ccc1OC] |
| 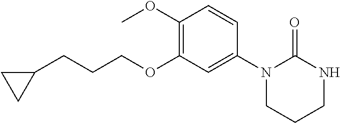 [SMILES: COc1ccc(N2CCCNC2=O)cc1OCCCC1CC1] | 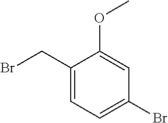 | 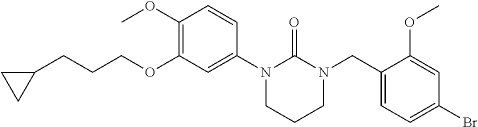 [SMILES: COc1cc(Br)ccc1CN1CCCN(c2ccc(OC)c(OCCCC3CC3)c2)C1=O] |
| 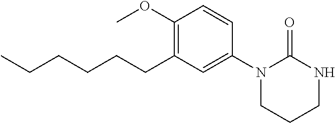 [SMILES: CCCCCCc1cc(N2CCCNC2=O)ccc1OC] | 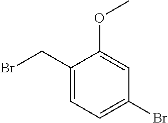 | 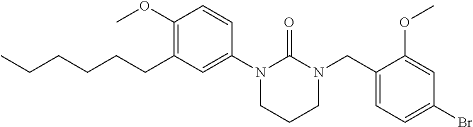 [SMILES: CCCCCCc1cc(N2CCCN(Cc3ccc(Br)cc3OC)C2=O)ccc1OC] |
| 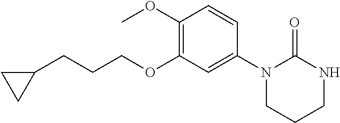 [SMILES: COc1ccc(N2CCCNC2=O)cc1OCCCC1CC1] | 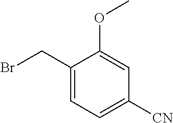 | 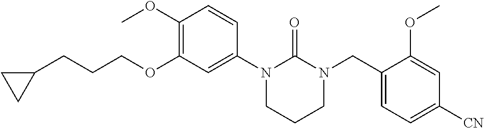 [SMILES: COc1cc(C#N)ccc1CN1CCCN(c2ccc(OC)c(OCCCC3CC3)c2)C1=O] |

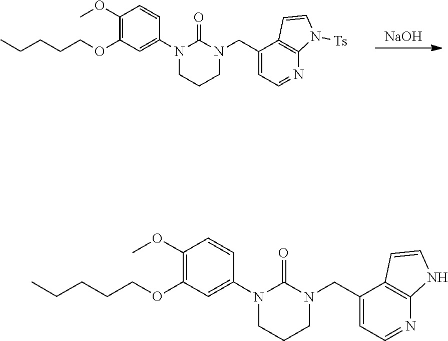 [SMILES: CCCCCOc1cc(N2CCCN(Cc3ccnc4c3ccn4S(=O)(=O)c3ccc(C)cc3)C2=O)ccc1OC ; "NaOH" ; CCCCCOc1cc(N2CCCN(Cc3ccnc4[nH]ccc34)C2=O)ccc1OC]

A solution of 1-(4-methoxy-3-(pentyloxy)phenyl)-3-((1-tosyl-1H-pyrrolo[2,3-b]pyridin-4-yl)methyl)tetrahydropyrimidin-2(1H)-one (43 g, 75 mmol) in anhydrous 2-methyltetrahydrofuran (880 mL) is warmed to 40° C. and stirred for 5 minutes. A 50% wt. aqeuous solution of sodium hydroxide (44 mL, 165 mmol) is added to the reaction mixture over 30 minutes at 4° C. The resulting mixture is stirred at 40° C. for 20 hours. 1M aqueous sodium hydroxide solution is added to the mixture at 40° C., then the aqueous phase is separated and extracted with ethyl acetate. The combined organic phases are washed with the 1M aqueous sodium hydroxide solution followed by water. The organic phase is dried over anhydrous sodium sulfate, filtered, and concentrated in vacuo to afford 1-((1H-pyrrolo[2,3-b]pyridin-4-yl)methyl)-3-(4-methoxy-3-(pentyloxy)phenyl)tetrahydropyrimidin-2(1H)-one.

The following compounds are prepared in a similar manner as described above.

| Tosylate | Compound |
|---|---|
| 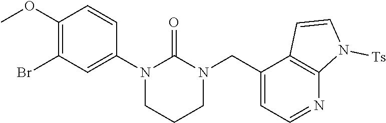 [SMILES: COc1ccc(N2CCCN(Cc3ccnc4c3ccn4S(=O)(=O)c3ccc(C)cc3)C2=O)cc1Br] | 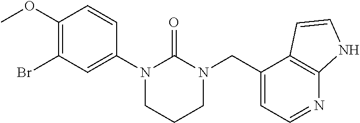 [SMILES: COc1ccc(N2CCCN(Cc3ccnc4[nH]ccc34)C2=O)cc1Br] |
| 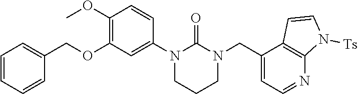 [SMILES: COc1ccc(N2CCCN(Cc3ccnc4c3ccn4S(=O)(=O)c3ccc(C)cc3)C2=O)cc1OCc1ccccc1] | 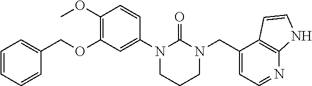 [SMILES: COc1ccc(N2CCCN(Cc3ccnc4[nH]ccc34)C2=O)cc1OCc1ccccc1] |
| 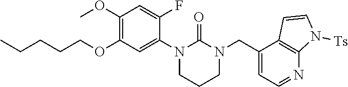 [SMILES: CCCCCOc1cc(N2CCCN(Cc3ccnc4c3ccn4S(=O)(=O)c3ccc(C)cc3)C2=O)c(F)cc1OC] | 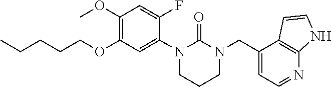 [SMILES: CCCCCOc1cc(N2CCCN(Cc3ccnc4[nH]ccc34)C2=O)c(F)cc1OC] |
| 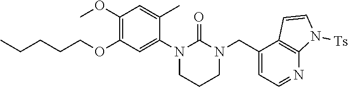 [SMILES: CCCCCOc1cc(N2CCCN(Cc3ccnc4c3ccn4S(=O)(=O)c3ccc(C)cc3)C2=O)c(C)cc1OC] | 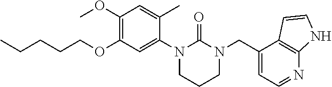 [SMILES: CCCCCOc1cc(N2CCCN(Cc3ccnc4[nH]ccc34)C2=O)c(C)cc1OC] |
| 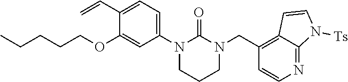 [SMILES: C=Cc1ccc(N2CCCN(Cc3ccnc4c3ccn4S(=O)(=O)c3ccc(C)cc3)C2=O)cc1OCCCCC] | 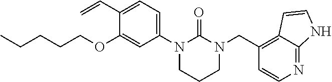 [SMILES: C=Cc1ccc(N2CCCN(Cc3ccnc4[nH]ccc34)C2=O)cc1OCCCCC] |
| 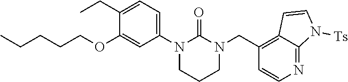 [SMILES: CCCCCOc1cc(N2CCCN(Cc3ccnc4c3ccn4S(=O)(=O)c3ccc(C)cc3)C2=O)ccc1CC] | 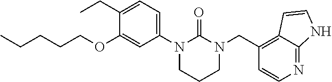 [SMILES: CCCCCOc1cc(N2CCCN(Cc3ccnc4[nH]ccc34)C2=O)ccc1CC] |
| 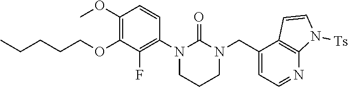 [SMILES: CCCCCOc1c(OC)ccc(N2CCCN(Cc3ccnc4c3ccn4S(=O)(=O)c3ccc(C)cc3)C2=O)c1F] | 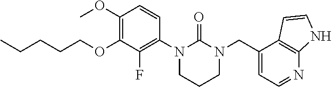 [SMILES: CCCCCOc1c(OC)ccc(N2CCCN(Cc3ccnc4[nH]ccc34)C2=O)c1F] |
| 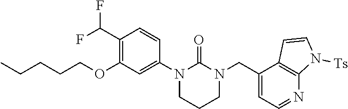 [SMILES: CCCCCOc1cc(N2CCCN(Cc3ccnc4c3ccn4S(=O)(=O)c3ccc(C)cc3)C2=O)ccc1C(F)F] | 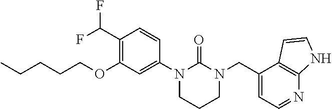 [SMILES: CCCCCOc1cc(N2CCCN(Cc3ccnc4[nH]ccc34)C2=O)ccc1C(F)F] |
| 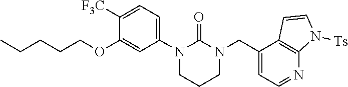 [SMILES: CCCCCOc1cc(N2CCCN(Cc3ccnc4c3ccn4S(=O)(=O)c3ccc(C)cc3)C2=O)ccc1C(F)(F)F] | 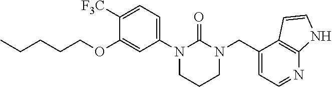 [SMILES: CCCCCOc1cc(N2CCCN(Cc3ccnc4[nH]ccc34)C2=O)ccc1C(F)(F)F] |
| 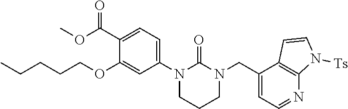 [SMILES: CCCCCOc1cc(N2CCCN(Cc3ccnc4c3ccn4S(=O)(=O)c3ccc(C)cc3)C2=O)ccc1C(=O)OC] | 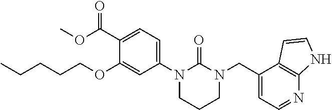 [SMILES: CCCCCOc1cc(N2CCCN(Cc3ccnc4[nH]ccc34)C2=O)ccc1C(=O)OC] |
| 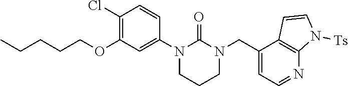 [SMILES: CCCCCOc1cc(N2CCCN(Cc3ccnc4c3ccn4S(=O)(=O)c3ccc(C)cc3)C2=O)ccc1Cl] | 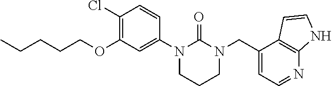 [SMILES: CCCCCOc1cc(N2CCCN(Cc3ccnc4[nH]ccc34)C2=O)ccc1Cl] |
| 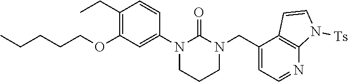 [SMILES: CCCCCOc1cc(N2CCCN(Cc3ccnc4c3ccn4S(=O)(=O)c3ccc(C)cc3)C2=O)ccc1CC] | 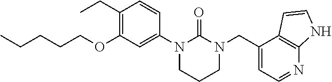 [SMILES: CCCCCOc1cc(N2CCCN(Cc3ccnc4[nH]ccc34)C2=O)ccc1CC] |
| 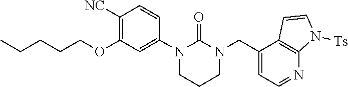 [SMILES: CCCCCOc1cc(N2CCCN(Cc3ccnc4c3ccn4S(=O)(=O)c3ccc(C)cc3)C2=O)ccc1C#N] | 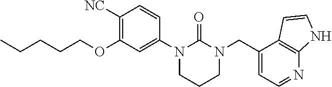 [SMILES: CCCCCOc1cc(N2CCCN(Cc3ccnc4[nH]ccc34)C2=O)ccc1C#N] |
| 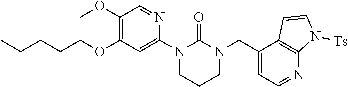 [SMILES: CCCCCOc1cc(N2CCCN(Cc3ccnc4c3ccn4S(=O)(=O)c3ccc(C)cc3)C2=O)ncc1OC] | 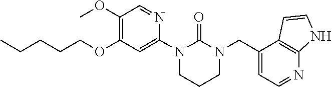 [SMILES: CCCCCOc1cc(N2CCCN(Cc3ccnc4[nH]ccc34)C2=O)ncc1OC] |
| 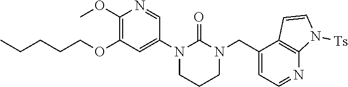 [SMILES: CCCCCOc1cc(N2CCCN(Cc3ccnc4c3ccn4S(=O)(=O)c3ccc(C)cc3)C2=O)cnc1OC] | 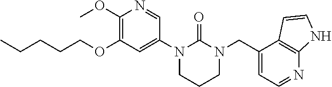 [SMILES: CCCCCOc1cc(N2CCCN(Cc3ccnc4[nH]ccc34)C2=O)cnc1OC] |
| 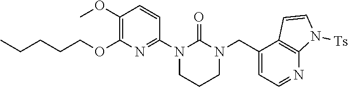 [SMILES: CCCCCOc1nc(N2CCCN(Cc3ccnc4c3ccn4S(=O)(=O)c3ccc(C)cc3)C2=O)ccc1OC] | 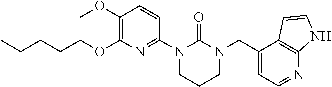 [SMILES: CCCCCOc1nc(N2CCCN(Cc3ccnc4[nH]ccc34)C2=O)ccc1OC] |
| 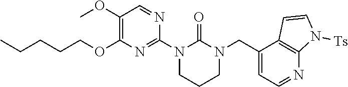 [SMILES: CCCCCOc1nc(N2CCCN(Cc3ccnc4c3ccn4S(=O)(=O)c3ccc(C)cc3)C2=O)ncc1OC] | 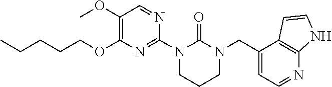 [SMILES: CCCCCOc1nc(N2CCCN(Cc3ccnc4[nH]ccc34)C2=O)ncc1OC] |
| 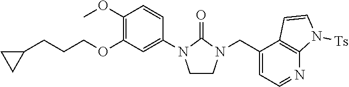 [SMILES: COc1ccc(N2CCN(Cc3ccnc4c3ccn4S(=O)(=O)c3ccc(C)cc3)C2=O)cc1OCCCC1CC1] | 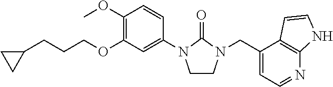 [SMILES: COc1ccc(N2CCN(Cc3ccnc4[nH]ccc34)C2=O)cc1OCCCC1CC1] |
| 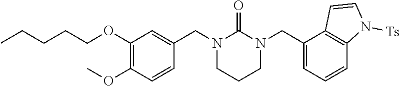 [SMILES: CCCCCOc1cc(CN2CCCN(Cc3cccc4c3ccn4S(=O)(=O)c3ccc(C)cc3)C2=O)ccc1OC] | 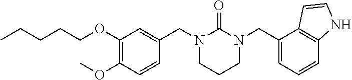 [SMILES: CCCCCOc1cc(CN2CCCN(Cc3cccc4[nH]ccc34)C2=O)ccc1OC] |
| 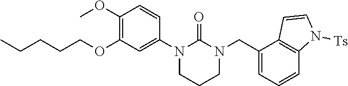 [SMILES: CCCCCOc1cc(N2CCCN(Cc3cccc4c3ccn4S(=O)(=O)c3ccc(C)cc3)C2=O)ccc1OC] | 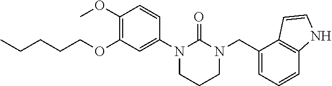 [SMILES: CCCCCOc1cc(N2CCCN(Cc3cccc4[nH]ccc34)C2=O)ccc1OC] |
| 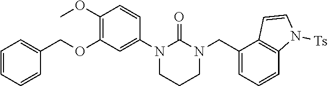 [SMILES: COc1ccc(N2CCCN(Cc3cccc4c3ccn4S(=O)(=O)c3ccc(C)cc3)C2=O)cc1OCc1ccccc1] | 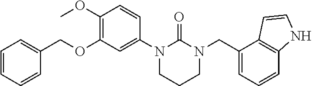 [SMILES: COc1ccc(N2CCCN(Cc3cccc4[nH]ccc34)C2=O)cc1OCc1ccccc1] |
| 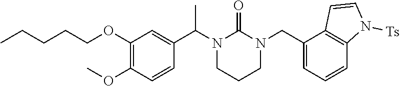 [SMILES: CCCCCOc1cc(C(C)N2CCCN(Cc3cccc4c3ccn4S(=O)(=O)c3ccc(C)cc3)C2=O)ccc1OC] | 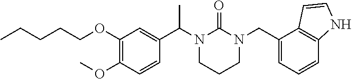 [SMILES: CCCCCOc1cc(C(C)N2CCCN(Cc3cccc4[nH]ccc34)C2=O)ccc1OC] |
| 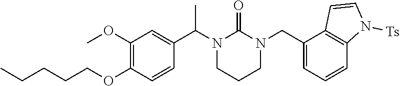 [SMILES: CCCCCOc1ccc(C(C)N2CCCN(Cc3cccc4c3ccn4S(=O)(=O)c3ccc(C)cc3)C2=O)cc1OC] | 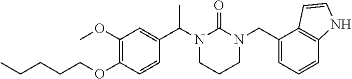 [SMILES: CCCCCOc1ccc(C(C)N2CCCN(Cc3cccc4[nH]ccc34)C2=O)cc1OC] |
| 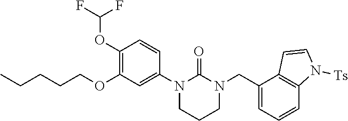 [SMILES: CCCCCOc1cc(N2CCCN(Cc3cccc4c3ccn4S(=O)(=O)c3ccc(C)cc3)C2=O)ccc1OC(F)F] | 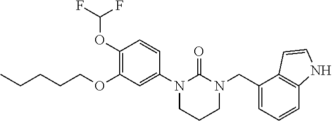 [SMILES: CCCCCOc1cc(N2CCCN(Cc3cccc4[nH]ccc34)C2=O)ccc1OC(F)F] |
| 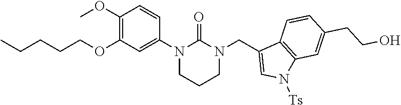 [SMILES: CCCCCOc1cc(N2CCCN(Cc3cn(S(=O)(=O)c4ccc(C)cc4)c4cc(CCO)ccc34)C2=O)ccc1OC] | 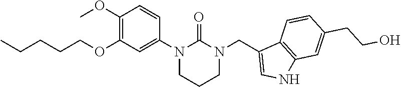 [SMILES: CCCCCOc1cc(N2CCCN(Cc3c[nH]c4cc(CCO)ccc34)C2=O)ccc1OC] |

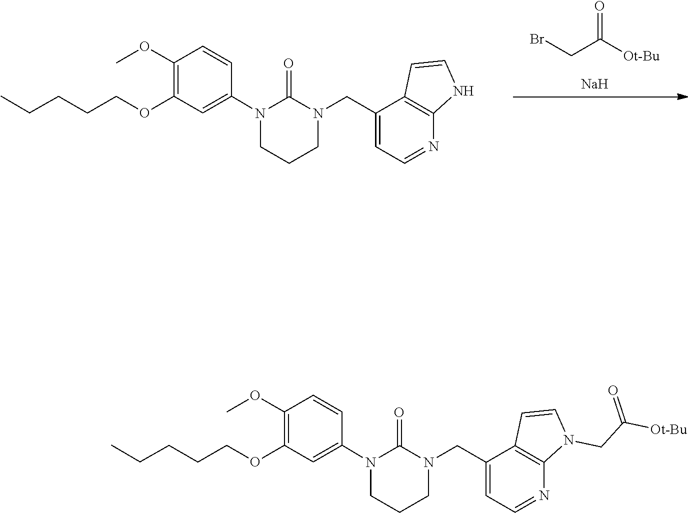

To a solution of (1-((1H-pyrrolo[2,3-b]pyridin-4-yl)methyl)-3-(4-methoxy-3-(pentyloxy)phenyl)tetrahydropyrimidin-2(1H)-one) (250 mg, 0.59 mmol) in anhydrous N,N-dimethylformamide (2 mL) is added sodium hydride (60 wt % in oil, 26 mg, 0.65 mmol) at room temperature. The mixture is stirred at room temperature for 10 min then cooled to 0° C., and a solution of tert-butyl bromoacetate (121 mg, 0.62 mmol) in N,N-dimethylformamide (1 mL) was added dropwise. The reaction is slowly warmed to room temperature and stirred for 3.5 hours. Water is slowly added to the reaction, and the resulting mixture is extracted with ethyl acetate. The combined organic layers are washed with brine, dried over anhydrous sodium sulfate, filtered, and concentrated in vacuo. The residue is purified by column chromatography over silica gel to afford tert-butyl 2-(4-((3-(4-methoxy-3-(pentyloxy)phenyl)-2-oxotetrahydropyrimidin-1(2H)-yl)methyl)-1H-pyrrolo[2,3-b]pyridin-1-yl)acetate).

The following compounds are prepared in a similar manner as described above.

| Heterocycle | Bromide | Compound |
|---|---|---|
| 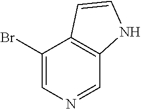 | 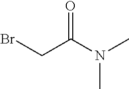 | 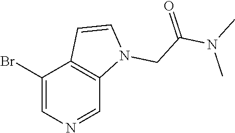 [SMILES: CN(C)C(=O)Cn1ccc2c(Br)cncc21] |
| 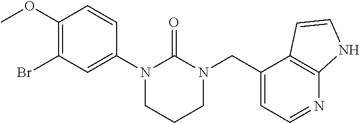 [SMILES: COc1ccc(N2CCCN(Cc3ccnc4[nH]ccc34)C2=O)cc1Br] | 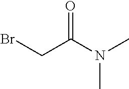 | 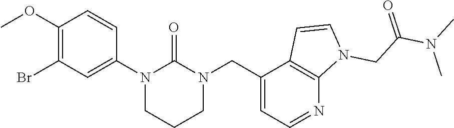 |
| 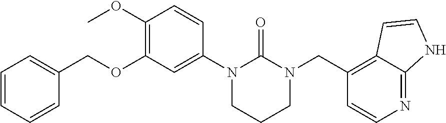 [SMILES: COc1ccc(N2CCCN(Cc3ccnc4[nH]ccc34)C2=O)cc1OCc1ccccc1] | 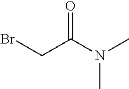 | 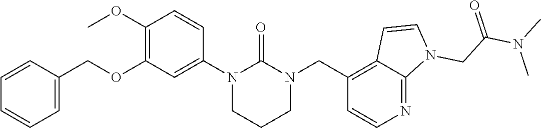 [SMILES: COc1ccc(N2CCCN(Cc3ccnc4c3ccn4CC(=O)N(C)C)C2=O)cc1OCc1ccccc1] |
| 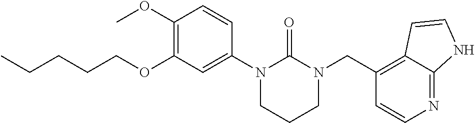 [SMILES: CCCCCOc1cc(N2CCCN(Cc3ccnc4[nH]ccc34)C2=O)ccc1OC] | 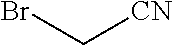 | 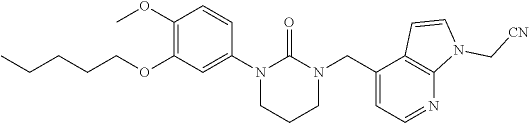 [SMILES: CCCCCOc1cc(N2CCCN(Cc3ccnc4c3ccn4CC#N)C2=O)ccc1OC] |
| 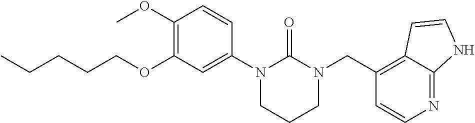 [SMILES: CCCCCOc1cc(N2CCCN(Cc3ccnc4[nH]ccc34)C2=O)ccc1OC] | 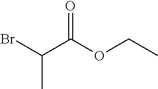 | 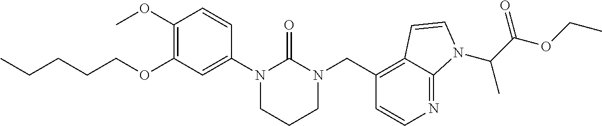 [SMILES: CCCCCOc1cc(N2CCCN(Cc3ccnc4c3ccn4C(C)C(=O)OCC)C2=O)ccc1OC] |
| 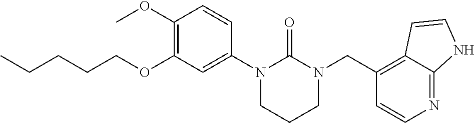 [SMILES: CCCCCOc1cc(N2CCCN(Cc3ccnc4[nH]ccc34)C2=O)ccc1OC] | 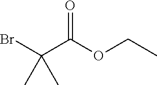 | 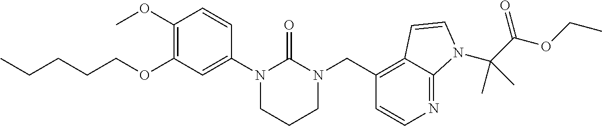 [SMILES: CCCCCOc1cc(N2CCCN(Cc3ccnc4c3ccn4C(C)(C)C(=O)OCC)C2=O)ccc1OC] |
| 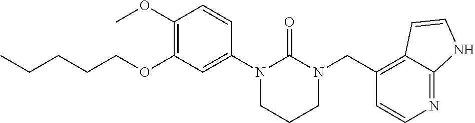 [SMILES: CCCCCOc1cc(N2CCCN(Cc3ccnc4[nH]ccc34)C2=O)ccc1OC] | 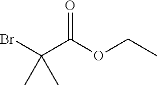 | 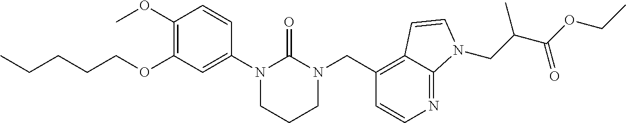 [SMILES: CCCCCOc1cc(N2CCCN(Cc3ccnc4c3ccn4CC(C)C(=O)OCC)C2=O)ccc1OC] |
| 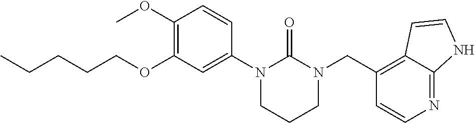 [SMILES: CCCCCOc1cc(N2CCCN(Cc3ccnc4[nH]ccc34)C2=O)ccc1OC] | 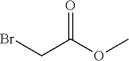 | 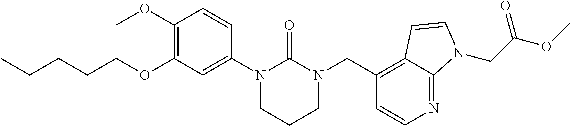 [SMILES: CCCCCOc1cc(N2CCCN(Cc3ccnc4c3ccn4CC(=O)OC)C2=O)ccc1OC] |
| 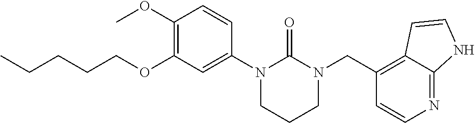 [SMILES: CCCCCOc1cc(N2CCCN(Cc3ccnc4[nH]ccc34)C2=O)ccc1OC] | 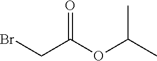 | 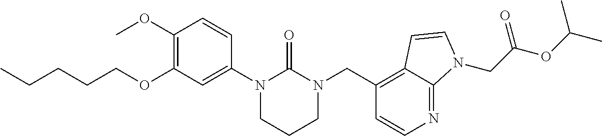 [SMILES: CCCCCOc1cc(N2CCCN(Cc3ccnc4c3ccn4CC(=O)OC(C)C)C2=O)ccc1OC] |
| 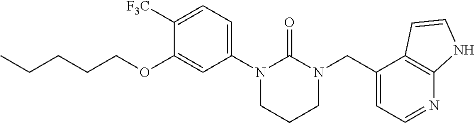 [SMILES: CCCCCOc1cc(N2CCCN(Cc3ccnc4[nH]ccc34)C2=O)ccc1C(F)(F)F] | 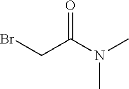 | 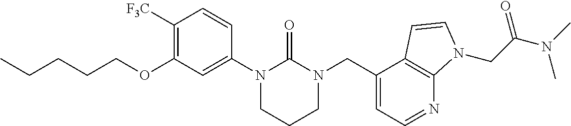 [SMILES: CCCCCOc1cc(N2CCCN(Cc3ccnc4c3ccn4CC(=O)N(C)C)C2=O)ccc1C(F)(F)F] |
| 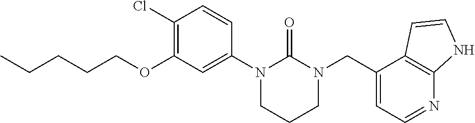 [SMILES: CCCCCOc1cc(N2CCCN(Cc3ccnc4[nH]ccc34)C2=O)ccc1Cl] | 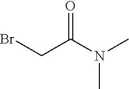 | 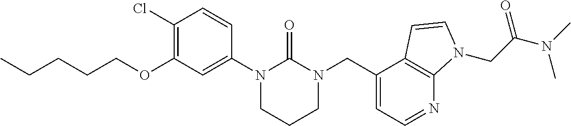 [SMILES: CCCCCOc1cc(N2CCCN(Cc3ccnc4c3ccn4CC(=O)N(C)C)C2=O)ccc1Cl] |
| 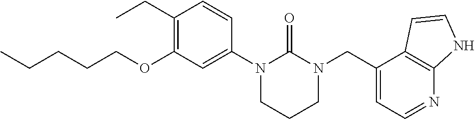 [SMILES: CCCCCOc1cc(N2CCCN(Cc3ccnc4[nH]ccc34)C2=O)ccc1CC] | 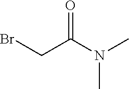 | 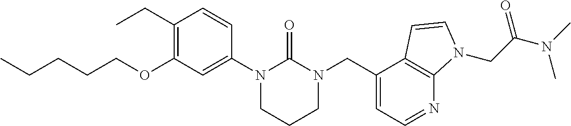 [SMILES: CCCCCOc1cc(N2CCCN(Cc3ccnc4c3ccn4CC(=O)N(C)C)C2=O)ccc1CC] |
| 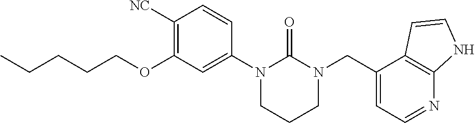 [SMILES: CCCCCOc1cc(N2CCCN(Cc3ccnc4[nH]ccc34)C2=O)ccc1C#N] | 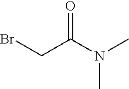 | 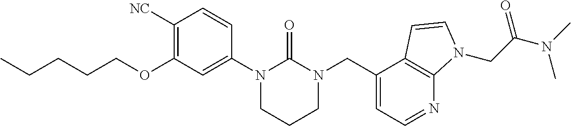 [SMILES: CCCCCOc1cc(N2CCCN(Cc3ccnc4c3ccn4CC(=O)N(C)C)C2=O)ccc1C#N] |
| 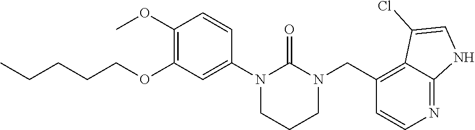 [SMILES: CCCCCOc1cc(N2CCCN(Cc3ccnc4[nH]cc(Cl)c34)C2=O)ccc1OC] | MeI | 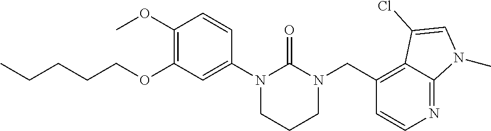 [SMILES: CCCCCOc1cc(N2CCCN(Cc3ccnc4c3c(Cl)cn4C)C2=O)ccc1OC] |
| 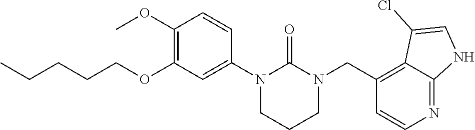 [SMILES: CCCCCOc1cc(N2CCCN(Cc3ccnc4[nH]cc(Cl)c34)C2=O)ccc1OC] | 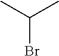 | 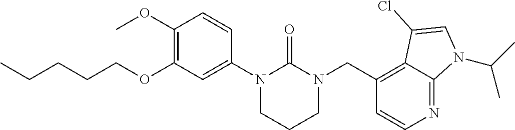 [SMILES: CCCCCOc1cc(N2CCCN(Cc3ccnc4c3c(Cl)cn4C(C)C)C2=O)ccc1OC] |
| 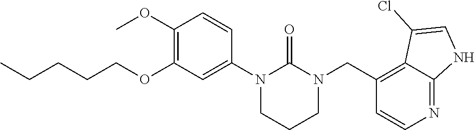 [SMILES: CCCCCOc1cc(N2CCCN(Cc3ccnc4[nH]cc(Cl)c34)C2=O)ccc1OC] | 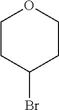 | 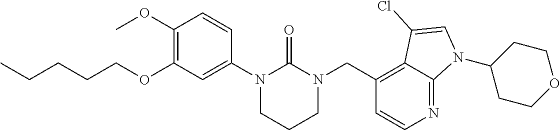 [SMILES: CCCCCOc1cc(N2CCCN(Cc3ccnc4c3c(Cl)cn4C3CCOCC3)C2=O)ccc1OC] |
| 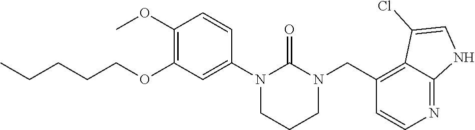 [SMILES: CCCCCOc1cc(N2CCCN(Cc3ccnc4[nH]cc(Cl)c34)C2=O)ccc1OC] | 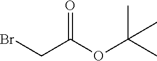 | 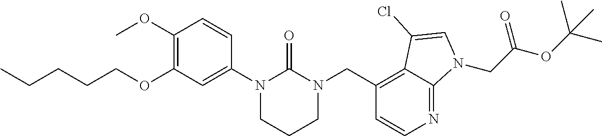 [SMILES: CCCCCOc1cc(N2CCCN(Cc3ccnc4c3c(Cl)cn4CC(=O)OC(C)(C)C)C2=O)ccc1OC] |
| 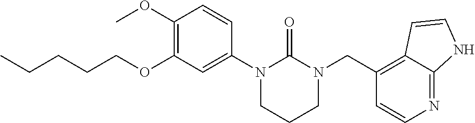 [SMILES: CCCCCOc1cc(N2CCCN(Cc3ccnc4[nH]ccc34)C2=O)ccc1OC] | 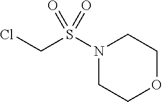 [SMILES: O=S(=O)(CCl)N1CCOCC1] | 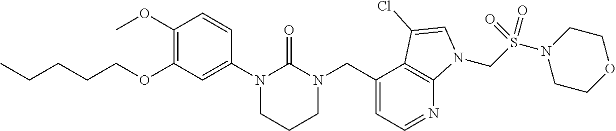 [SMILES: CCCCCOc1cc(N2CCCN(Cc3ccnc4c3c(Cl)cn4CS(=O)(=O)N3CCOCC3)C2=O)ccc1OC] |
| 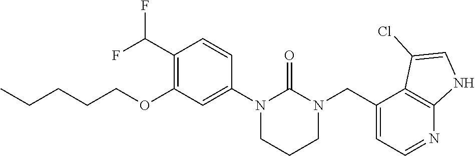 [SMILES: CCCCCOc1cc(N2CCCN(Cc3ccnc4[nH]cc(Cl)c34)C2=O)ccc1C(F)F] | 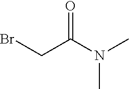 | 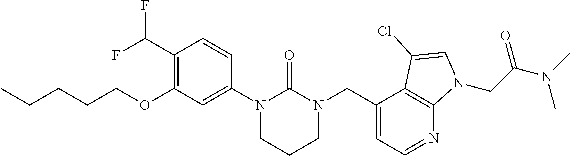 [SMILES: CCCCCOc1cc(N2CCCN(Cc3ccnc4c3c(Cl)cn4CC(=O)N(C)C)C2=O)ccc1C(F)F] |
| 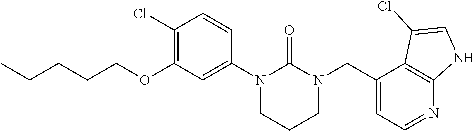 [SMILES: CCCCCOc1cc(N2CCCN(Cc3ccnc4[nH]cc(Cl)c34)C2=O)ccc1Cl] | 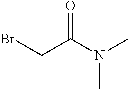 | 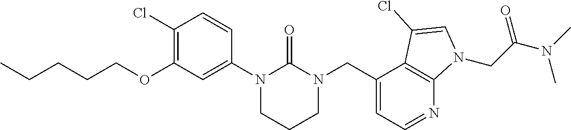 [SMILES: CCCCCOc1cc(N2CCCN(Cc3ccnc4c3c(Cl)cn4CC(=O)N(C)C)C2=O)ccc1Cl] |
| 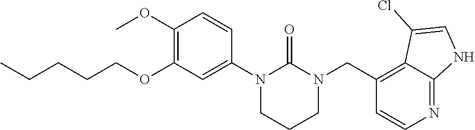 [SMILES: CCCCCOc1cc(N2CCCN(Cc3ccnc4[nH]cc(Cl)c34)C2=O)ccc1OC] | 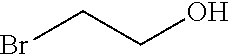 | 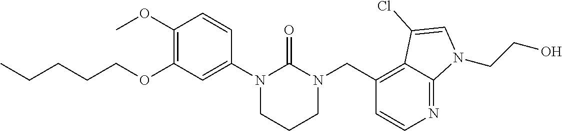 [SMILES: CCCCCOc1cc(N2CCCN(Cc3ccnc4c3c(Cl)cn4CCO)C2=O)ccc1OC] |
| 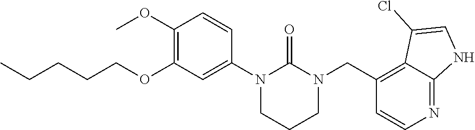 [SMILES: CCCCCOc1cc(N2CCCN(Cc3ccnc4[nH]cc(Cl)c34)C2=O)ccc1OC] | 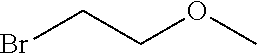 | 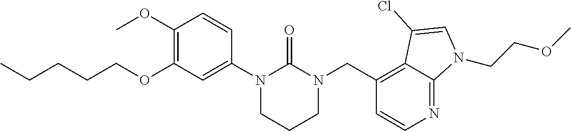 [SMILES: CCCCCOc1cc(N2CCCN(Cc3ccnc4c3c(Cl)cn4CCOC)C2=O)ccc1OC] |
| 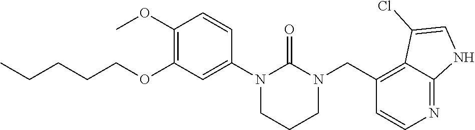 [SMILES: CCCCCOc1cc(N2CCCN(Cc3ccnc4[nH]cc(Cl)c34)C2=O)ccc1OC] | 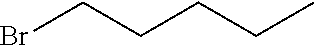 | 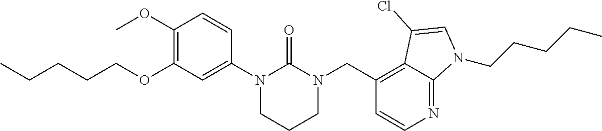 [SMILES: CCCCCOc1cc(N2CCCN(Cc3ccnc4c3c(Cl)cn4CCCCC)C2=O)ccc1OC] |
| 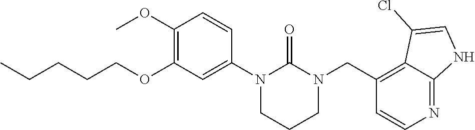 [SMILES: CCCCCOc1cc(N2CCCN(Cc3ccnc4[nH]cc(Cl)c34)C2=O)ccc1OC] | 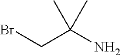 | 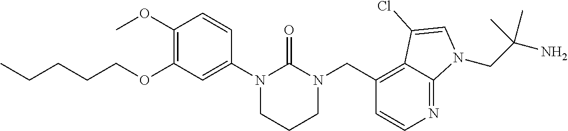 [SMILES: CCCCCOc1cc(N2CCCN(Cc3ccnc4c3c(Cl)cn4CC(C)(C)N)C2=O)ccc1OC] |
| 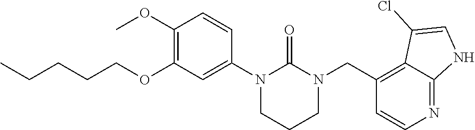 [SMILES: CCCCCOc1cc(N2CCCN(Cc3ccnc4[nH]cc(Cl)c34)C2=O)ccc1OC] | 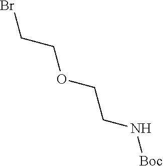 [SMILES: CC(C)(C)OC(=O)NCCOCCBr] | 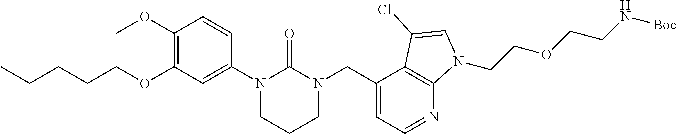 [SMILES: CCCCCOc1cc(N2CCCN(Cc3ccnc4c3c(Cl)cn4CCOCCNC(=O)OC(C)(C)C)C2=O)ccc1OC] |
| 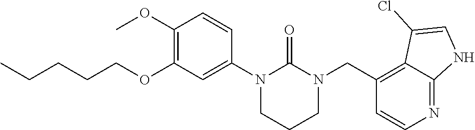 [SMILES: CCCCCOc1cc(N2CCCN(Cc3ccnc4[nH]cc(Cl)c34)C2=O)ccc1OC] | 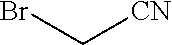 | 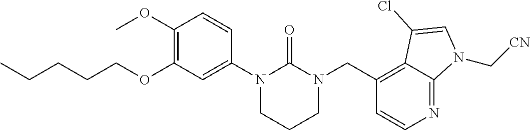 [SMILES: CCCCCOc1cc(N2CCCN(Cc3ccnc4c3c(Cl)cn4CC#N)C2=O)ccc1OC] |
| 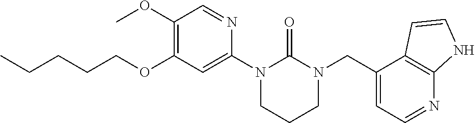 [SMILES: CCCCCOc1cc(N2CCCN(Cc3ccnc4[nH]ccc34)C2=O)ncc1OC] | 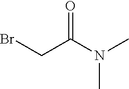 | 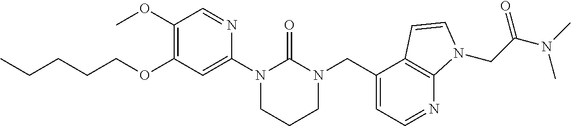 [SMILES: CCCCCOc1cc(N2CCCN(Cc3ccnc4c3ccn4CC(=O)N(C)C)C2=O)ncc1OC] |
| 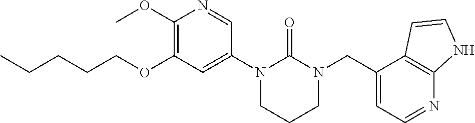 [SMILES: CCCCCOc1cc(N2CCCN(Cc3ccnc4[nH]ccc34)C2=O)cnc1OC] | 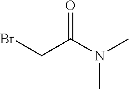 | 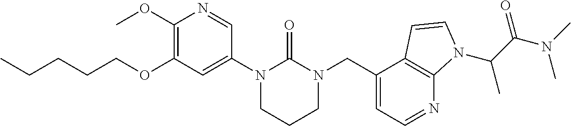 [SMILES: CCCCCOc1cc(N2CCCN(Cc3ccnc4c3ccn4C(C)C(=O)N(C)C)C2=O)cnc1OC] |
| 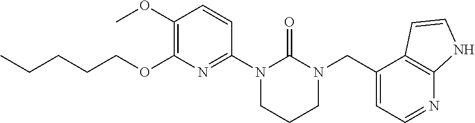 [SMILES: CCCCCOc1nc(N2CCCN(Cc3ccnc4[nH]ccc34)C2=O)ccc1OC] | 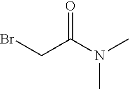 | 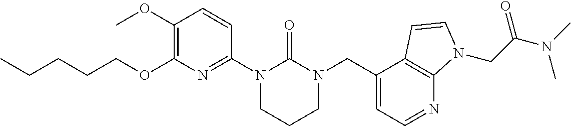 [SMILES: CCCCCOc1nc(N2CCCN(Cc3ccnc4c3ccn4CC(=O)N(C)C)C2=O)ccc1OC] |
| 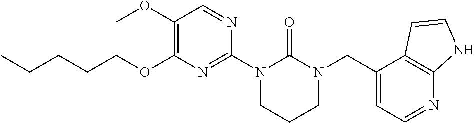 [SMILES: CCCCCOc1nc(N2CCCN(Cc3ccnc4[nH]ccc34)C2=O)ncc1OC] | 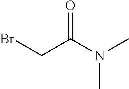 | 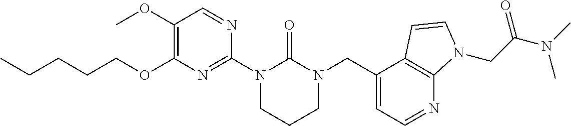 [SMILES: CCCCCOc1nc(N2CCCN(Cc3ccnc4c3ccn4CC(=O)N(C)C)C2=O)ncc1OC] |
| 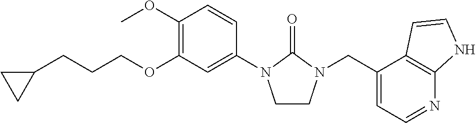 [SMILES: COc1ccc(N2CCN(Cc3ccnc4[nH]ccc34)C2=O)cc1OCCCC1CC1] | 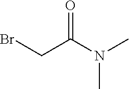 | 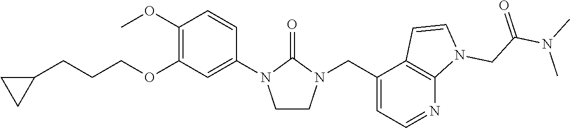 [SMILES: COc1ccc(N2CCN(Cc3ccnc4c3ccn4CC(=O)N(C)C)C2=O)cc1OCCCC1CC1] |
| 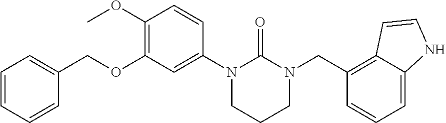 [SMILES: COc1ccc(N2CCCN(Cc3cccc4[nH]ccc34)C2=O)cc1OCc1ccccc1] | 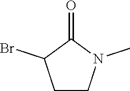 | 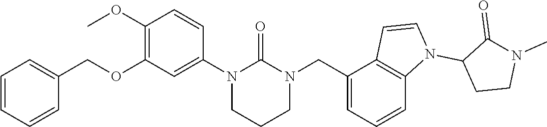 [SMILES: COc1ccc(N2CCCN(Cc3cccc4c3ccn4C3CCN(C)C3=O)C2=O)cc1OCc1ccccc1] |
| 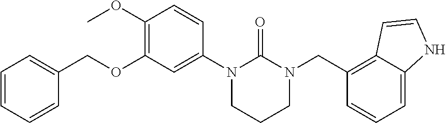 [SMILES: COc1ccc(N2CCCN(Cc3cccc4[nH]ccc34)C2=O)cc1OCc1ccccc1] | 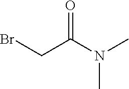 | 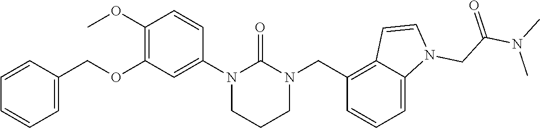 [SMILES: COc1ccc(N2CCCN(Cc3cccc4c3ccn4CC(=O)N(C)C)C2=O)cc1OCc1ccccc1] |
| 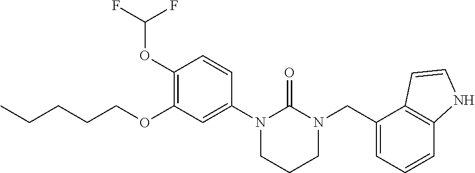 [SMILES: CCCCCOc1cc(N2CCCN(Cc3cccc4[nH]ccc34)C2=O)ccc1OC(F)F] | 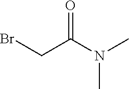 | 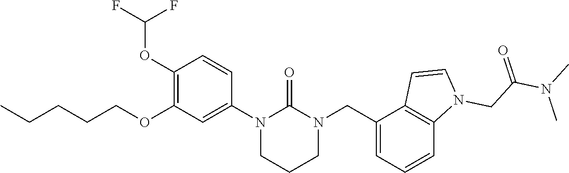 [SMILES: CCCCCOc1cc(N2CCCN(Cc3cccc4c3ccn4CC(=O)N(C)C)C2=O)ccc1OC(F)F] |
| 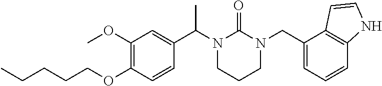 [SMILES: CCCCCOc1ccc(C(C)N2CCCN(Cc3cccc4[nH]ccc34)C2=O)cc1OC] | 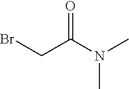 | 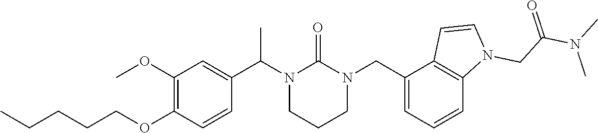 [SMILES: CCCCCOc1ccc(C(C)N2CCCN(Cc3cccc4c3ccn4CC(=O)N(C)C)C2=O)cc1OC] |
| 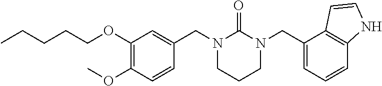 [SMILES: CCCCCOc1cc(CN2CCCN(Cc3cccc4[nH]ccc34)C2=O)ccc1OC] | 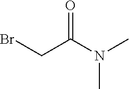 | 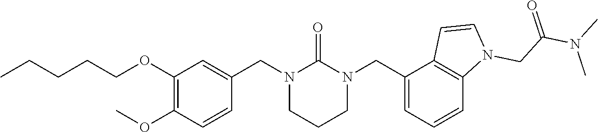 [SMILES: CCCCCOc1cc(CN2CCCN(Cc3cccc4c3ccn4CC(=O)N(C)C)C2=O)ccc1OC] |
| 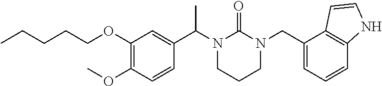 [SMILES: CCCCCOc1cc(C(C)N2CCCN(Cc3cccc4[nH]ccc34)C2=O)ccc1OC] | 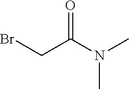 | 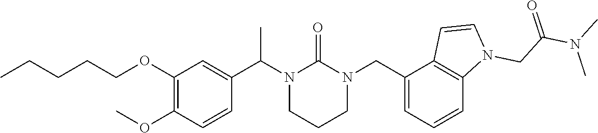 [SMILES: CCCCCOc1cc(C(C)N2CCCN(Cc3cccc4c3ccn4CC(=O)N(C)C)C2=O)ccc1OC] |

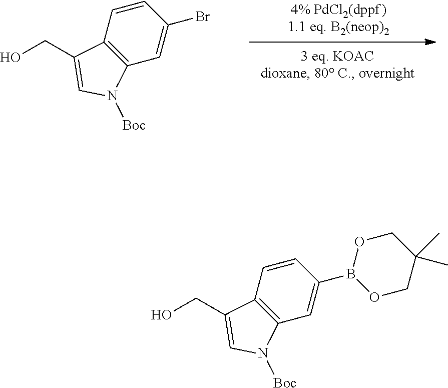 [SMILES: CC(C)(C)OC(=O)n1cc(CO)c2ccc(Br)cc21 ; CC1(C)COB(c2ccc3c(CO)cn(C(=O)OC(C)(C)C)c3c2)OC1]

A round bottom flask is charged with 1,1′-bis(diphenylphosphino)ferrocene dichlorodpalladium(II) (45 mg, 0.062 mmol), potassium acetate (456 mg, 4.60 mmol), tert-butyl 6-bromo-3-(hydroxymethyl)-1H-indole-1-carboxylate (500 mg, 1.53 mmol), and bis(neopentyl glycolato)diboron (401 mg, 1.69 mmol). The flask is evacuated and purged with nitrogen three times. Anhydrous 1,4-dioxane (4.6 mL) is added. The reaction is heated to 80° Celsius and stirred at this temperature overnight. The reaction is cooled to room temperature, then filtered through CELITE®. The filter cake is thoroughly washed with ethyl acetate. The filtered solution is concentrated in vacuo to afford tert-butyl 6-(5,5-dimethyl-1,3,2-dioxaborinan-2-yl)-3-(hydroxymethyl)-1H-indole-1-carboxylate.

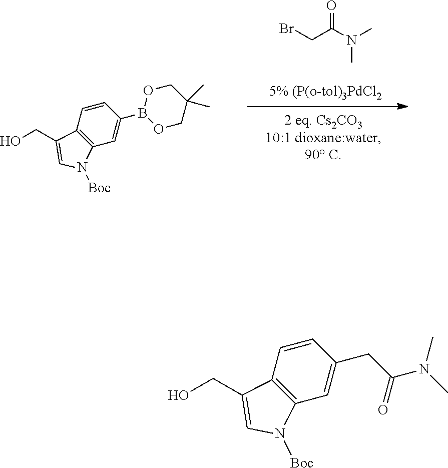

A round bottom flask is charged with cesium carbonate (914 mg, 2.78 mmol), dichlorobis(tri-o-tolylphosphine)-palladium(II) (56.4 mg, 0.070 mmol), tert-butyl 6-(5,5-dimethyl-1,3,2-dioxaborinan-2-yl)-3-(hydroxymethyl)-1H-indole-1-carboxylate (500 mg, 1.39 mmol). The flask evacuated and purged with nitrogen three times. Anhydrous 1,4-dioxane (2.5 mL), and Water (253 uL) are added followed by 2-bromo-N,N-dimethylacetamide (306 uL, 2.78 mmol). The reaction is heated to 90° C. and stirred at this temperature for 1 hour. After cooling to room temperature, saturated aqueous ammonium chloride solution is added, and the resulting mixture is extracted with ethyl acetate. The combined organic layers are washed with brine, then dried over anhydrous sodium sulfate, filtered, and concentrated in vacuo. Purification by column chromatography over reversed-phase C18 silica gel afforded tert-butyl 6-(2-(dimethylamino)-2-oxoethyl)-3-(hydroxymethyl)-1H-indole-1-carboxylate.

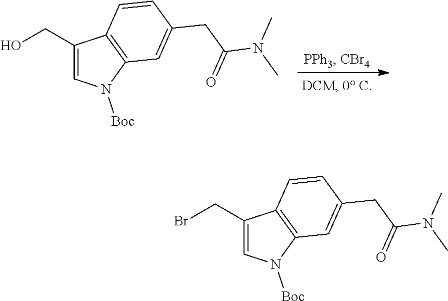 [SMILES: CN(C)C(=O)Cc1ccc2c(CO)cn(C(=O)OC(C)(C)C)c2c1 ; CN(C)C(=O)Cc1ccc2c(CBr)cn(C(=O)OC(C)(C)C)c2c1]

To a solution of tert-butyl 6-(2-(dimethylamino)-2-oxoethyl)-3-(hydroxymethyl)-1H-indole-1-carboxylate (200 mg, 0.60 mmol) in anhydrous dichloromethane (1.8 mL) at 0° C. is added triphenylphosphine (158 mg, 0.60 mmol), followed by carbon tetrabromide (202 mg, 0.60 mmol). The reaction is stirred for 30 minutes at 0° C. Silica gel (1.5 g) is added, and the reaction is carefully concentrated in vacuo. Purification by column chromatography over silica gel afforded tert-butyl 3-(bromomethyl)-6-(2-(dimethylamino)-2-oxoethyl)-1H-indole-1-carboxylate.

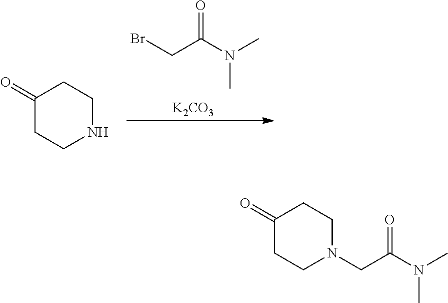

To a solution of piperidin-4-one (2.00 g, 20 mmol) in anhydrous N,N-dimethylformamide (50 mL) is added anhydrous potassium carbonate (3.60 g, 26 mmol) and 2-bromo-N,N-dimethylacetamide (3.65 g, 22 mmol) at room temperature. The reaction is stirred at room temperature overnight. Water is added, then the reaction is extracted with ethyl acetate. The combined organic layers are washed with brine, then dried over sodium sulfate, filtered, and concentrated in vacuo. Purification by column chromatography over silica gel afforded N,N-dimethyl-2-(4-oxopiperidin-1-yl)acetamide.

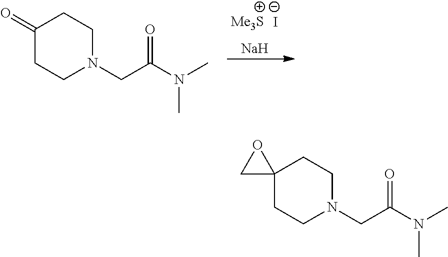

To a solution of trimethylsulfonium iodide (1.18 g, 5.8 mmol) in anhydrous dimethyl sulfoxide (10 mL) was added sodium hydride (60 wt % in oil, 0.23 g, 5.8 mmol) at room temperature. The mixture is stirred at room temperature for 1 hour before N,N-dimethyl-2-(4-oxopiperidin-1-yl)acetamide (1.0 g, 5.4 mmol) is added. The mixture is stirred for another hour at room temperature then quenched with water. The reaction is extracted with ethyl acetate. The combined organic extracts are washed with brine then dried over sodium sulfate, filtered, and concentrated in vacuo. Purification by column chromatography over silica gel afforded N,N-dimethyl-2-(1-oxa-6-azaspiro[2.5]octan-6-yl)acetamide.

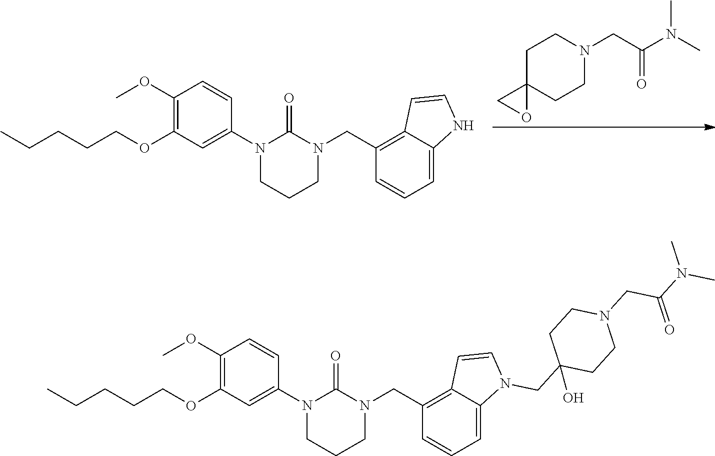 [SMILES: CCCCCOc1cc(N2CCCN(Cc3cccc4[nH]ccc34)C2=O)ccc1OC ; CN(C)C(=O)CN1CCC2(CC1)CO2 ; CCCCCOc1cc(N2CCCN(Cc3cccc4c3ccn4CC3(O)CCN(CC(=O)N(C)C)CC3)C2=O)ccc1OC]

To a stirring solution of 1-((1H-indol-4-yl)methyl)-3-(4-methoxy-3-(pentyloxy)phenyl)tetrahydroprimidin-2(1H)-one (0.42 g, 1.0 mmol) in anhydrous N,N-dimethylformamide (10 mL) at 0° C. is added sodium hydride (60 wt % in oil, 80 mg, 2.0 mmol). The resulting slurry is stirred at 0° C. for 30 minutes. A solution N,N-dimethyl-2-(1-oxa-6-azaspiro[2.5]octan-6-yl)acetamide (0.21 g, 1.0 mmol) in N,N-dimethylformamide (2 mL) is added at 0° C. The reaction is warmed to 60° C. then stirred for 30 minutes at this temperature. The reaction is cooled to room temperature then quenched with water. The reaction is extracted with dichloromethane. The combined organic layers are washed with brine then dried over anhydrous sodium sulfate, filtered, and concentrated in vacuo. Purification by reversed-phase column chromatography over C18 silica gel afforded 2-(4-hydroxy-4-((4-((3-(4-methoxy-3-(pentyloxy)phenyl)-2-oxotetrahydropyrimidin-1(2H)-yl)methyl)-1H-indol-1-yl)methyl)piperidin-1-yl)-N,N-dimethylacetamide.

The following compounds are prepared in a similar manner as described above.

| Heterocycle | Epoxide | Compound |
|---|---|---|
| 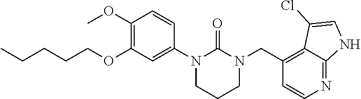 [SMILES: CCCCCOc1cc(N2CCCN(Cc3ccnc4[nH]cc(Cl)c34)C2=O)ccc1OC] | 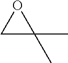 | 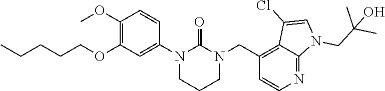 [SMILES: CCCCCOc1cc(N2CCCN(Cc3ccnc4c3c(Cl)cn4CC(C)(C)O)C2=O)ccc1OC] |

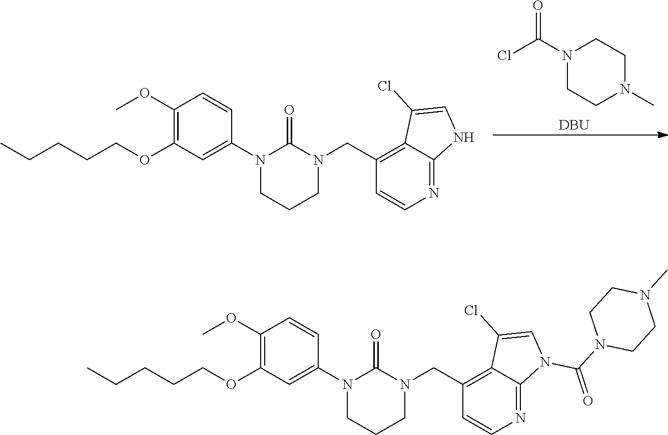

1-((3-chloro-1H-pyrrolo[2,3-b]pyridin-4-yl)methyl)-3-(4-methoxy-3-(pentyloxy)phenyl)tetrahydropyrimidin-2(1H)-one (55 mg, 0.12 mmol) is dissolved in anhydrous dichloromethane (1.2 mL) and cooled to 0° C. 1,8-Diazabicyclo[5.4.0]undec-7-ene (DBU, 45 uL, 0.30 mmol) is added, followed by 4-methylpiperazine-1-carbonyl chloride hydrochloride (26 mg, 0.13 mmol). The reaction is stirred at 0° C. for 3 hours. Saturated aqueous sodium bicarbonate solution is added, then the reaction is extracted with dichloromethane. The combined organic layers are dried over anhydrous sodium sulfate, filtered, and concentrated in vacuo. Purification by column chromatography over silica gel afforded 1-((3-chloro-1-(4-methylpiperazine-1-carbonyl)-1H-pyrrolo[2,3-b]pyridin-4-yl)methyl)-3-(4-methoxy-3-(pentyloxy)phenyl)tetrahydropyrimidin-2(1H)-one.

The following compounds are prepared in a similar manner as described above.

| Amine | Acid | Compound |
|---|---|---|
| 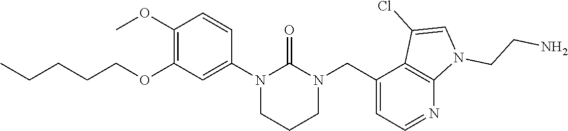 [SMILES: CCCCCOc1cc(N2CCCN(Cc3ccnc4c3c(Cl)cn4CCN)C2=O)ccc1OC] | 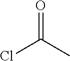 | 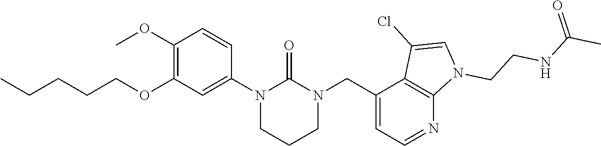 [SMILES: CCCCCOc1cc(N2CCCN(Cc3ccnc4c3c(Cl)cn4CCNC(C)=O)C2=O)ccc1OC] |
| 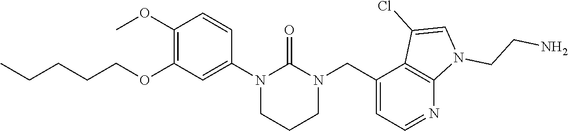 [SMILES: CCCCCOc1cc(N2CCCN(Cc3ccnc4c3c(Cl)cn4CCN)C2=O)ccc1OC] | 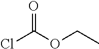 | 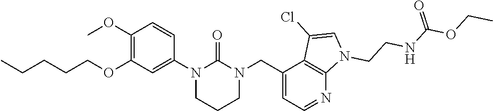 [SMILES: CCCCCOc1cc(N2CCCN(Cc3ccnc4c3c(Cl)cn4CCNC(=O)OCC)C2=O)ccc1OC] |
| 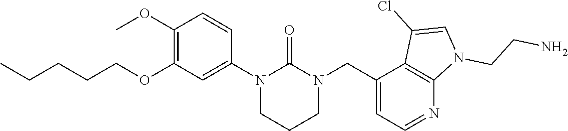 [SMILES: CCCCCOc1cc(N2CCCN(Cc3ccnc4c3c(Cl)cn4CCN)C2=O)ccc1OC] | 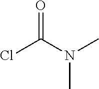 | 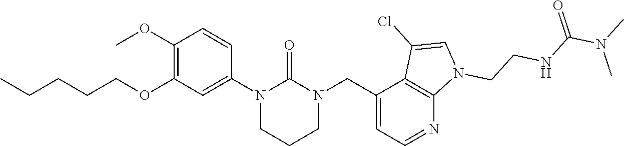 [SMILES: CCCCCOc1cc(N2CCCN(Cc3ccnc4c3c(Cl)cn4CCNC(=O)N(C)C)C2=O)ccc1OC] |
| 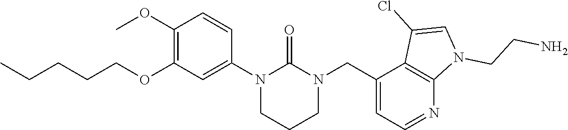 [SMILES: CCCCCOc1cc(N2CCCN(Cc3ccnc4c3c(Cl)cn4CCN)C2=O)ccc1OC] | 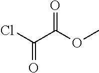 | 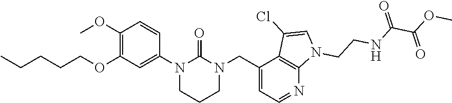 [SMILES: CCCCCOc1cc(N2CCCN(Cc3ccnc4c3c(Cl)cn4CCNC(=O)C(=O)OC)C2=O)ccc1OC] |
| 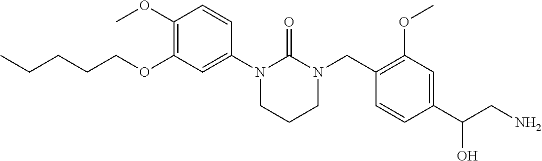 [SMILES: CCCCCOc1cc(N2CCCN(Cc3ccc(C(O)CN)cc3OC)C2=O)ccc1OC] | 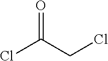 | 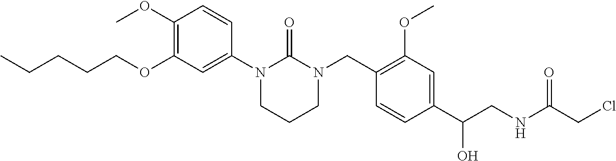 [SMILES: CCCCCOc1cc(N2CCCN(Cc3ccc(C(O)CNC(=O)CCl)cc3OC)C2=O)ccc1OC] |

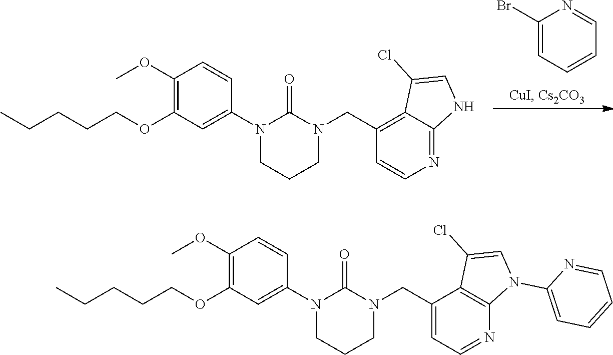 [SMILES: CCCCCOc1cc(N2CCCN(Cc3ccnc4[nH]cc(Cl)c34)C2=O)ccc1OC ; Brc1ccccn1 ; CCCCCOc1cc(N2CCCN(Cc3ccnc4c3c(Cl)cn4-c3ccccn3)C2=O)ccc1OC]

A 10-mL sealed tube is charged with 1-((3-chloro-1H-pyrrolo[2,3-b]pyridin-4-yl)methyl)-3-(4-methoxy-3-(pentyloxy)phenyl)tetrahydropyrimidin-2(1H)-one (200 mg, 0.44 mmol), cuprous iodide (8 mg, 0.04 mmol), and anhydrous cesium carbonate (430 mg, 1.32 mmol). The tube is evacuated and purged with argon three times. Anhydrous N,N-dimethylformamide (4.4 mL) and 2-bromopyridine (84 uL, 0.88 mmol) are added. The tube is sealed under argon and stirred at 150° C. overnight. The reaction is cooled to room temperature then diluted with water. The reaction is extracted with ethyl acetate. The combined organic layers are dried over anhydrous sodium sulfate, filtered, and concentrated in vacuo. Purification by column chromatography over reversed-phase C18 silica gel afforded 1-((3-chloro-1-(pyridin-2-yl)-1H-pyrrolo[2,3-b]pyridin-4-yl)methyl)-3-(4-methoxy-3-(pentyloxy)phenyl)tetrahydropyrimidin-2(1H)-one.

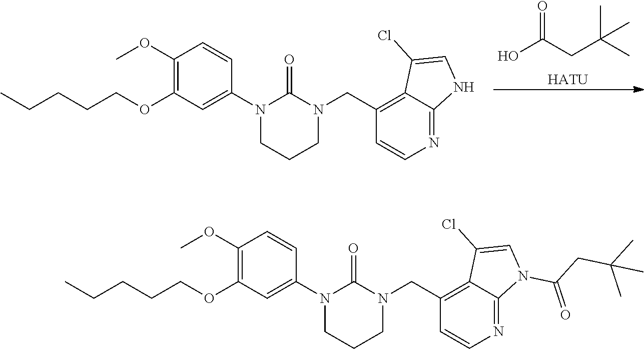

To a solution of 1-((3-chloro-1H-pyrrolo[2,3-b]pyridin-4-yl)methyl)-3-(4-methoxy-3-(pentyloxy)phenyl)tetrahydropyrimidin-2(1H)-one (50.0 mg, 0.11 mmol) and 3,3-dimethylbutanoic acid (19.1 mg, 0.16 mmol) in N,N-dimethylformamide (3 mL) is added N-[(dimethylamino)-1H-1,2,3-triazolo-[4,5-b]pyridin-1-ylmethylene]-N-methylmethanaminium hexafluorophosphate N-oxide (HATU, 62.4 mg, 0.16 mmol) and triethylamine (45 uL, 0.32 mmol). The reaction mixture is heated to 60° C. and stirred at this temperature for 12 hours. The reaction is quenched with a minimal amount of water and purified directly by preparative HPLC to afford 1-((3-chloro-1-(3,3-dimethylbutanoyl)-1H-pyrrolo[2,3-b]pyridin-4-yl)methyl)-3-(4-methoxy-3-(pentyloxy)phenyl)tetrahydropyrimidin-2(1H)-one

The following compounds are prepared in a similar manner as described above.

| Heterocycle | Acid | Compound |
|---|---|---|
| 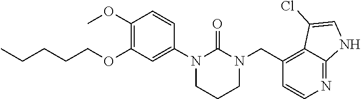 [SMILES: CCCCCOc1cc(N2CCCN(Cc3ccnc4[nH]cc(Cl)c34)C2=O)ccc1OC] | 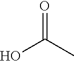 | 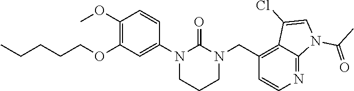 [SMILES: CCCCCOc1cc(N2CCCN(Cc3ccnc4c3c(Cl)cn4C(C)=O)C2=O)ccc1OC] |

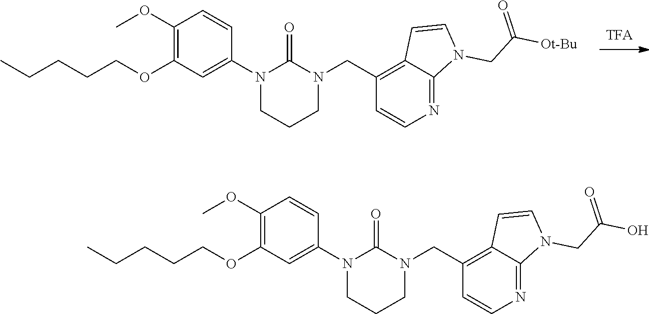 [SMILES: CCCCCOc1cc(N2CCCN(Cc3ccnc4c3ccn4CC(=O)OC(C)(C)C)C2=O)ccc1OC ; CCCCCOc1cc(N2CCCN(Cc3ccnc4c3ccn4CC(=O)O)C2=O)ccc1OC]

Neat trifluoroacetic acid (2 mL) is cooled to 0° C. tert-Butyl 2-(4-((3-(4-methoxy-3-(pentyloxy)phenyl)-2-oxotetrahydropyrimidin-1(2H)-yl)methyl)-1H-pyrrolo[2,3-b]pyridin-1-yl)acetate (250 mg, 0.47 mmol) is added in portions at 0° C. The reaction mixture is stirred at 0° C. for 10 min, then warmed to room temperature and stirred for 2 hours. The volatiles are removed in vacuo. Trituration with diethyl ether afforded 2-(4-((3-(4-methoxy-3-(pentyloxy)phenyl)-2-oxotetrahydropyrimidin-1(2H)-yl)methyl)-1H-pyrrolo[2,3-b]pyridin-1-yl)acetic acid.

The following compounds are prepared in a similar manner as described above.

| Ester | Compound |
|---|---|
| 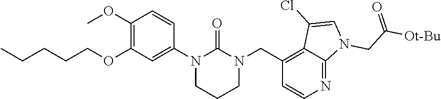 [SMILES: CCCCCOc1cc(N2CCCN(Cc3ccnc4c3c(Cl)cn4CC(=O)OC(C)(C)C)C2=O)ccc1OC] | 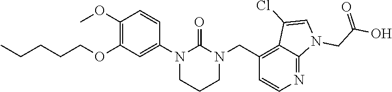 [SMILES: CCCCCOc1cc(N2CCCN(Cc3ccnc4c3c(Cl)cn4CC(=O)O)C2=O)ccc1OC] |
| 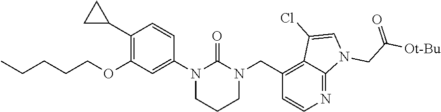 [SMILES: CCCCCOc1cc(N2CCCN(Cc3ccnc4c3c(Cl)cn4CC(=O)OC(C)(C)C)C2=O)ccc1C1CC1] | 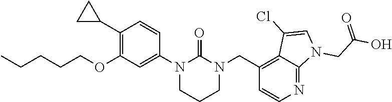 [SMILES: CCCCCOc1cc(N2CCCN(Cc3ccnc4c3c(Cl)cn4CC(=O)O)C2=O)ccc1C1CC1] |
| 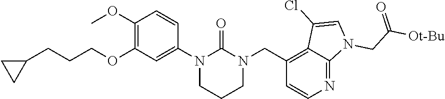 [SMILES: COc1ccc(N2CCCN(Cc3ccnc4c3c(Cl)cn4CC(=O)OC(C)(C)C)C2=O)cc1OCCCC1CC1] | 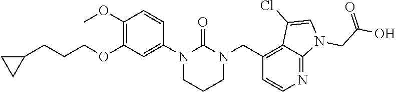 [SMILES: COc1ccc(N2CCCN(Cc3ccnc4c3c(Cl)cn4CC(=O)O)C2=O)cc1OCCCC1CC1] |
| 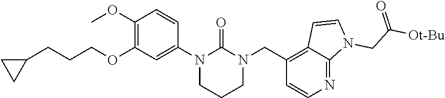 [SMILES: COc1ccc(N2CCCN(Cc3ccnc4c3ccn4CC(=O)OC(C)(C)C)C2=O)cc1OCCCC1CC1] | 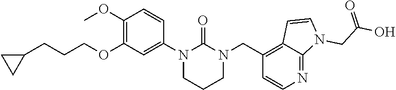 [SMILES: COc1ccc(N2CCCN(Cc3ccnc4c3ccn4CC(=O)O)C2=O)cc1OCCCC1CC1] |

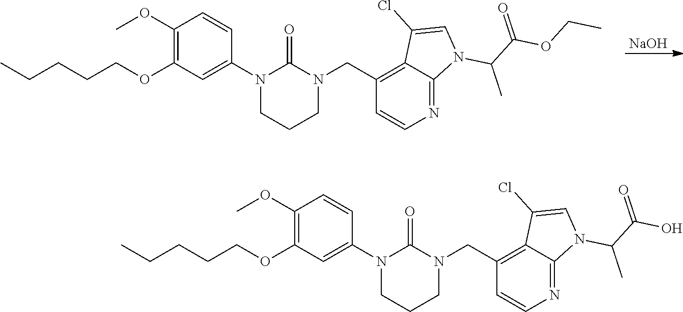

Ethyl 2-(3-chloro-4-((3-(4-methoxy-3-(pentyloxy)phenyl)-2-oxotetrahydropyrimidin-1(2H)-yl)methyl)-1H-pyrrolo[2,3-b]pyridin-1-yl)propanoate (60 mg, 0.11 mmol) is dissolved in 1:1 v/v methanol/dichloromethane (2 mL). Powdered sodium hydroxide (9 mg, 0.22 mmol) is added in a single portion. The reaction is stirred at room temperature for 4 hours. The reaction is quenched with the addition of water, then washed with dichloromethane. The organic washes are discarded, then the remaining aqueous layer is treated with 1M hydrochloric acid until pH<3. The acidified aqueous layer is then extracted with dichloromethane. These organic extracts are dried over sodium sulfate, filtered, and concentrated in vacuo to afford 2-(3-chloro-4-((3-(4-methoxy-3-(pentyloxy)phenyl)-2-oxotetrahydropyrimidin-1(2H)-yl)methyl)-1H-pyrrolo[2,3-b]pyridin-1-yl)propanoic acid.

The following compounds are prepared in a similar manner as described above.

| Ester | Compound |
|---|---|
| 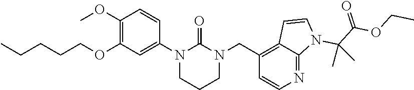 [SMILES: CCCCCOc1cc(N2CCCN(Cc3ccnc4c3ccn4C(C)(C)C(=O)OCC)C2=O)ccc1OC] | 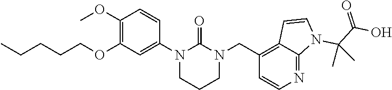 [SMILES: CCCCCOc1cc(N2CCCN(Cc3ccnc4c3ccn4C(C)(C)C(=O)O)C2=O)ccc1OC] |
| 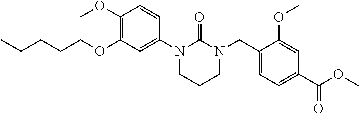 [SMILES: CCCCCOc1cc(N2CCCN(Cc3ccc(C(=O)OC)cc3OC)C2=O)ccc1OC] | 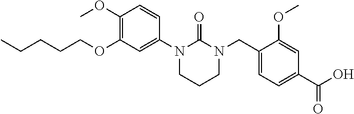 [SMILES: CCCCCOc1cc(N2CCCN(Cc3ccc(C(=O)O)cc3OC)C2=O)ccc1OC] |

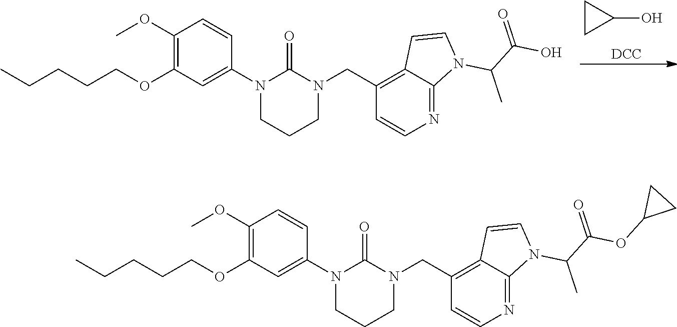

A solution of 2-(4-((3-(4-methoxy-3-(pentyloxy)phenyl)-2-oxotetrahydropyrimidin-1(2H)-yl)methyl)-1H-pyrrolo[2,3-b]pyridin-1-yl)propanoic acid (55 mg, 0.11 mmol) in anhydrous dichloromethane (1 mL) is treated with N,N′-dicyclohexylcarbodiimide (DCC, 30 mg, 0.14 mmol), 4-(dimethylamino)pyridine (1 mg, 0.01 mmol), and cyclopropanol (7 mg, 0.12 mmol). The reaction is stirred at room temperature overnight. Water is added, and the reaction is extracted with dichloromethane. The combined organic extracts are dried over anhydrous sodium sulfate, filtered, and concentrated in vacuo. Purification by column chromatography over silica gel afforded cyclopropyl 2-(4-((3-(4-methoxy-3-(pentyloxy)phenyl)-2-oxotetrahydropyrimidin-1(2H)-yl)methyl)-1H-pyrrolo[2,3-b]pyridin-1-yl)propanoate.

The following compounds are prepared in a similar manner as described above.

| Acid | Alcohol | Compound |
|---|---|---|
| 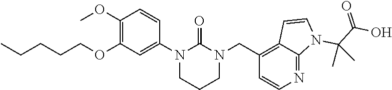 [SMILES: CCCCCOc1cc(N2CCCN(Cc3ccnc4c3ccn4C(C)(C)C(=O)O)C2=O)ccc1OC] | 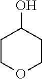 | 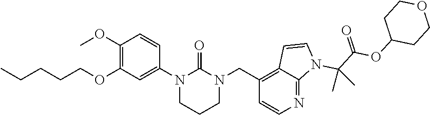 [SMILES: CCCCCOc1cc(N2CCCN(Cc3ccnc4c3ccn4C(C)(C)C(=O)OC3CCOCC3)C2=O)ccc1OC] |

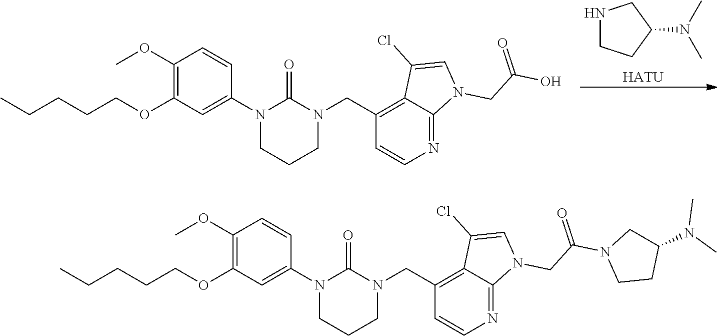

2-(3-chloro-4-((3-(4-methoxy-3-(pentyl oxy)phenyl)-2-oxotetrahydropyrimidin-1(2H)-yl)methyl)-1H-pyrrolo[2,3-b]pyridine-1-yl)acetic acid (25 mg, 0.05 mmol) is dissolved in N, N-dimethyl formamide (1 mL), then N-[(dimethylamino)-1H-1,2,3-triazolo-[4,5-b]pyridin-1-ylmethylene]-N-methylmethanaminium hexafluorophosphate N-oxide (HATU, 22 mg, 0.06 mmol), (R)—N,N-dimethylpyrrolidin-3-amine (6 mg, 0.05 mmol), and N,N-diisopropylethylamine (13 uL, 0.07 mmol) are added. The reaction mixture is stirred at room temperature for 2 hours. Water is added, and the reaction mixture is extracted with ethyl acetate. The organic layers are dried over anhydrous sodium sulfate, filtered, and concentrated in, vacuo. Purification by column chromatography over silica afforded ((R)-1-((3-chloro-1-(2-(3-(dimethyl amino)pyrrolidine-1-yl)-2-oxoethyl)-1H-pyrrolo[2,3-b]pyridine-4-yl)methyl)-3-(4-methoxy-3-(pentyloxy)phenyl)tetrahydropyrimidin-2(1H)-one).

The following compounds are prepared in a similar manner as described above.

| Acid | Amine | Compound |
|---|---|---|
| 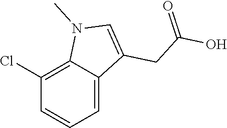 | 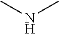 | 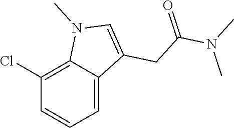 [SMILES: CN(C)C(=O)Cc1cn(C)c2c(Cl)cccc12] |
| 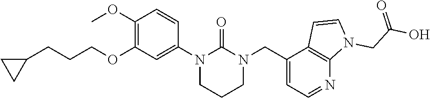 [SMILES: COc1ccc(N2CCCN(Cc3ccnc4c3ccn4CC(=O)O)C2=O)cc1OCCCC1CC1] | 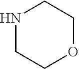 | 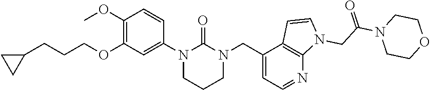 [SMILES: COc1ccc(N2CCCN(Cc3ccnc4c3ccn4CC(=O)N3CCOCC3)C2=O)cc1OCCCC1CC1] |
| 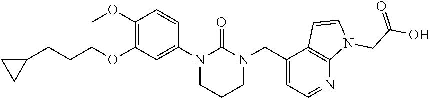 [SMILES: COc1ccc(N2CCCN(Cc3ccnc4c3ccn4CC(=O)O)C2=O)cc1OCCCC1CC1] | 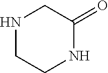 | 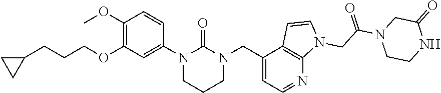 [SMILES: COc1ccc(N2CCCN(Cc3ccnc4c3ccn4CC(=O)N3CCNC(=O)C3)C2=O)cc1OCCCC1CC1] |
| 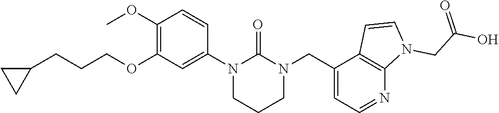 [SMILES: COc1ccc(N2CCCN(Cc3ccnc4c3ccn4CC(=O)O)C2=O)cc1OCCCC1CC1] | 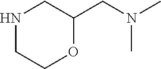 | 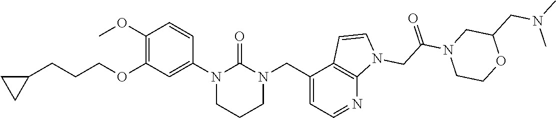 [SMILES: COc1ccc(N2CCCN(Cc3ccnc4c3ccn4CC(=O)N3CCOC(CN(C)C)C3)C2=O)cc1OCCCC1CC1] |
| 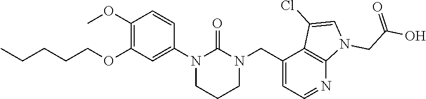 [SMILES: CCCCCOc1cc(N2CCCN(Cc3ccnc4c3c(Cl)cn4CC(=O)O)C2=O)ccc1OC] | 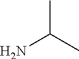 | 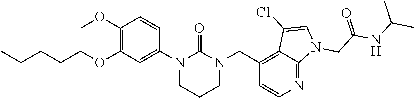 [SMILES: CCCCCOc1cc(N2CCCN(Cc3ccnc4c3c(Cl)cn4CC(=O)NC(C)C)C2=O)ccc1OC] |
| 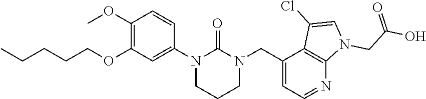 [SMILES: CCCCCOc1cc(N2CCCN(Cc3ccnc4c3c(Cl)cn4CC(=O)O)C2=O)ccc1OC] | H₂N—NH₂ | 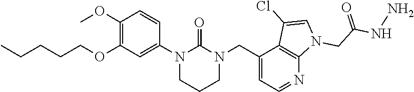 [SMILES: CCCCCOc1cc(N2CCCN(Cc3ccnc4c3c(Cl)cn4CC(=O)NN)C2=O)ccc1OC] |
| 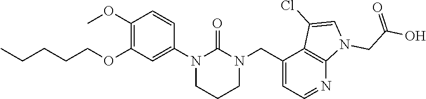 [SMILES: CCCCCOc1cc(N2CCCN(Cc3ccnc4c3c(Cl)cn4CC(=O)O)C2=O)ccc1OC] | 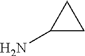 | 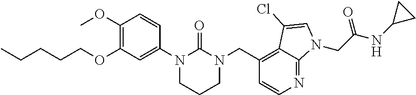 [SMILES: CCCCCOc1cc(N2CCCN(Cc3ccnc4c3c(Cl)cn4CC(=O)NC3CC3)C2=O)ccc1OC] |
| 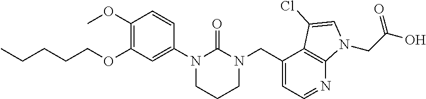 [SMILES: CCCCCOc1cc(N2CCCN(Cc3ccnc4c3c(Cl)cn4CC(=O)O)C2=O)ccc1OC] | 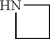 | 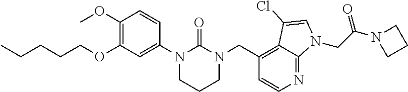 [SMILES: CCCCCOc1cc(N2CCCN(Cc3ccnc4c3c(Cl)cn4CC(=O)N3CCC3)C2=O)ccc1OC] |
| 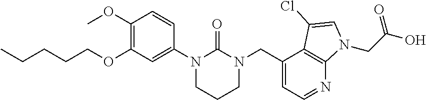 [SMILES: CCCCCOc1cc(N2CCCN(Cc3ccnc4c3c(Cl)cn4CC(=O)O)C2=O)ccc1OC] | 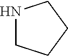 | 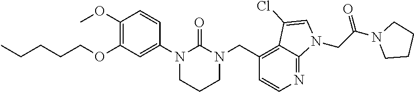 [SMILES: CCCCCOc1cc(N2CCCN(Cc3ccnc4c3c(Cl)cn4CC(=O)N3CCCC3)C2=O)ccc1OC] |
| 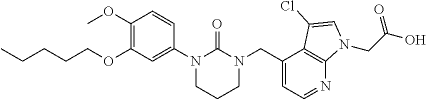 [SMILES: CCCCCOc1cc(N2CCCN(Cc3ccnc4c3c(Cl)cn4CC(=O)O)C2=O)ccc1OC] | 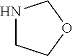 | 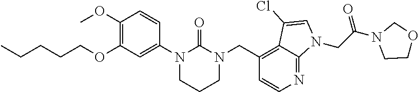 [SMILES: CCCCCOc1cc(N2CCCN(Cc3ccnc4c3c(Cl)cn4CC(=O)N3CCOC3)C2=O)ccc1OC] |
| 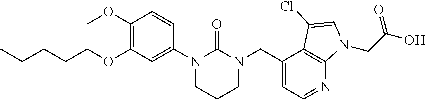 [SMILES: CCCCCOc1cc(N2CCCN(Cc3ccnc4c3c(Cl)cn4CC(=O)O)C2=O)ccc1OC] | 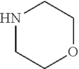 | 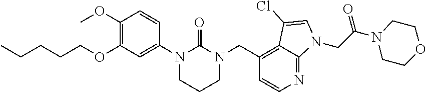 [SMILES: CCCCCOc1cc(N2CCCN(Cc3ccnc4c3c(Cl)cn4CC(=O)N3CCOCC3)C2=O)ccc1OC] |
| 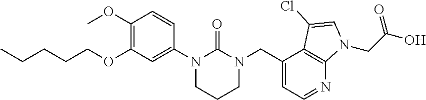 [SMILES: CCCCCOc1cc(N2CCCN(Cc3ccnc4c3c(Cl)cn4CC(=O)O)C2=O)ccc1OC] | 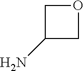 | 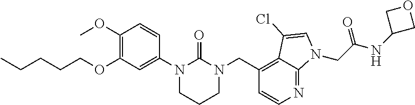 [SMILES: CCCCCOc1cc(N2CCCN(Cc3ccnc4c3c(Cl)cn4CC(=O)NC3COC3)C2=O)ccc1OC] |
| 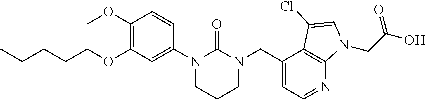 [SMILES: CCCCCOc1cc(N2CCCN(Cc3ccnc4c3c(Cl)cn4CC(=O)O)C2=O)ccc1OC] | 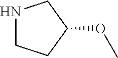 | 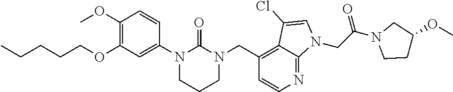 [SMILES: CCCCCOc1cc(N2CCCN(Cc3ccnc4c3c(Cl)cn4CC(=O)N3CC[C@@H](OC)C3)C2=O)ccc1OC] |
| 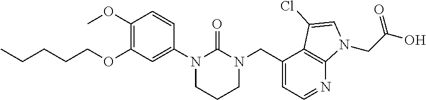 [SMILES: CCCCCOc1cc(N2CCCN(Cc3ccnc4c3c(Cl)cn4CC(=O)O)C2=O)ccc1OC] | 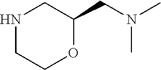 | 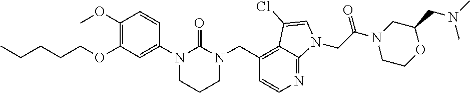 [SMILES: CCCCCOc1cc(N2CCCN(Cc3ccnc4c3c(Cl)cn4CC(=O)N3CCO[C@H](CN(C)C)C3)C2=O)ccc1OC] |
| 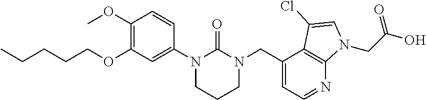 [SMILES: CCCCCOc1cc(N2CCCN(Cc3ccnc4c3c(Cl)cn4CC(=O)O)C2=O)ccc1OC] | 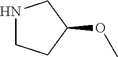 | 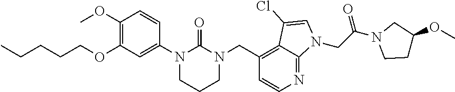 [SMILES: CCCCCOc1cc(N2CCCN(Cc3ccnc4c3c(Cl)cn4CC(=O)N3CC[C@H](OC)C3)C2=O)ccc1OC] |
| 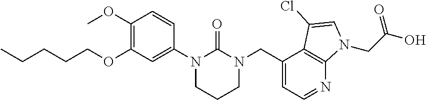 [SMILES: CCCCCOc1cc(N2CCCN(Cc3ccnc4c3c(Cl)cn4CC(=O)O)C2=O)ccc1OC] | 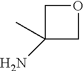 | 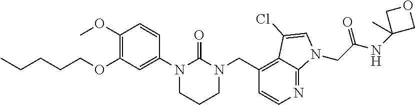 [SMILES: CCCCCOc1cc(N2CCCN(Cc3ccnc4c3c(Cl)cn4CC(=O)NC3(C)COC3)C2=O)ccc1OC] |
| 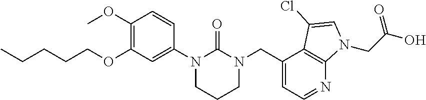 [SMILES: CCCCCOc1cc(N2CCCN(Cc3ccnc4c3c(Cl)cn4CC(=O)O)C2=O)ccc1OC] | 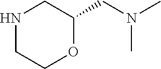 | 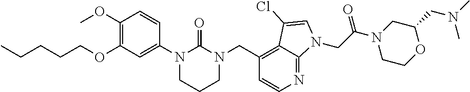 [SMILES: CCCCCOc1cc(N2CCCN(Cc3ccnc4c3c(Cl)cn4CC(=O)N3CCO[C@@H](CN(C)C)C3)C2=O)ccc1OC] |
| 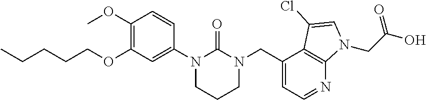 [SMILES: CCCCCOc1cc(N2CCCN(Cc3ccnc4c3c(Cl)cn4CC(=O)O)C2=O)ccc1OC] | 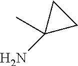 | 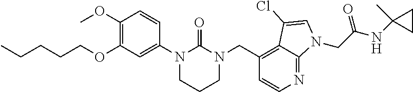 [SMILES: CCCCCOc1cc(N2CCCN(Cc3ccnc4c3c(Cl)cn4CC(=O)NC3(C)CC3)C2=O)ccc1OC] |
| 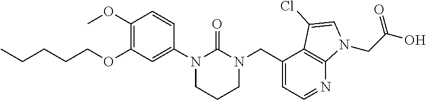 [SMILES: CCCCCOc1cc(N2CCCN(Cc3ccnc4c3c(Cl)cn4CC(=O)O)C2=O)ccc1OC] | 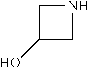 | 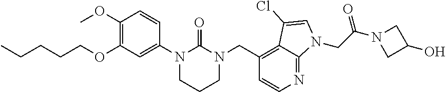 [SMILES: CCCCCOc1cc(N2CCCN(Cc3ccnc4c3c(Cl)cn4CC(=O)N3CC(O)C3)C2=O)ccc1OC] |
| 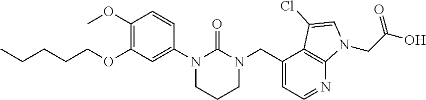 [SMILES: CCCCCOc1cc(N2CCCN(Cc3ccnc4c3c(Cl)cn4CC(=O)O)C2=O)ccc1OC] | 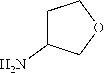 | 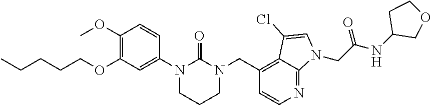 [SMILES: CCCCCOc1cc(N2CCCN(Cc3ccnc4c3c(Cl)cn4CC(=O)NC3CCOC3)C2=O)ccc1OC] |
| 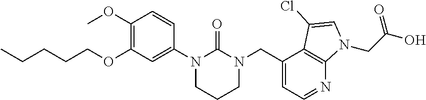 [SMILES: CCCCCOc1cc(N2CCCN(Cc3ccnc4c3c(Cl)cn4CC(=O)O)C2=O)ccc1OC] | 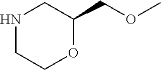 | 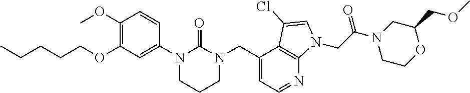 [SMILES: CCCCCOc1cc(N2CCCN(Cc3ccnc4c3c(Cl)cn4CC(=O)N3CCO[C@H](COC)C3)C2=O)ccc1OC] |
| 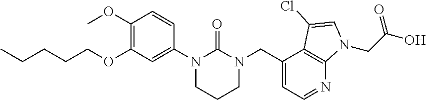 [SMILES: CCCCCOc1cc(N2CCCN(Cc3ccnc4c3c(Cl)cn4CC(=O)O)C2=O)ccc1OC] | 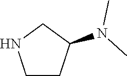 | 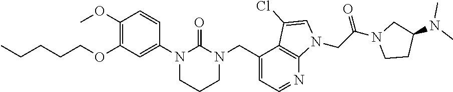 [SMILES: CCCCCOc1cc(N2CCCN(Cc3ccnc4c3c(Cl)cn4CC(=O)N3CC[C@H](N(C)C)C3)C2=O)ccc1OC] |
| 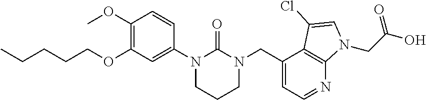 [SMILES: CCCCCOc1cc(N2CCCN(Cc3ccnc4c3c(Cl)cn4CC(=O)O)C2=O)ccc1OC] | 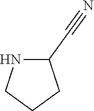 | 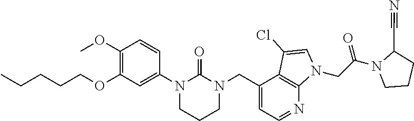 [SMILES: CCCCCOc1cc(N2CCCN(Cc3ccnc4c3c(Cl)cn4CC(=O)N3CCCC3C#N)C2=O)ccc1OC] |
| 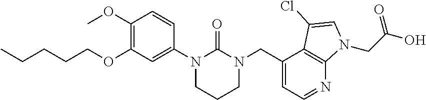 [SMILES: CCCCCOc1cc(N2CCCN(Cc3ccnc4c3c(Cl)cn4CC(=O)O)C2=O)ccc1OC] | 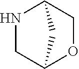 | 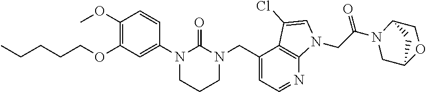 [SMILES: CCCCCOc1cc(N2CCCN(Cc3ccnc4c3c(Cl)cn4CC(=O)N3C[C@H]4C[C@@H]3CO4)C2=O)ccc1OC] |
| 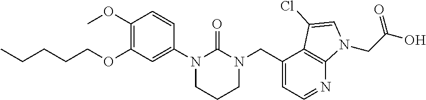 [SMILES: CCCCCOc1cc(N2CCCN(Cc3ccnc4c3c(Cl)cn4CC(=O)O)C2=O)ccc1OC] | 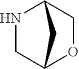 | 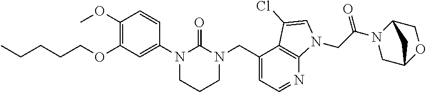 [SMILES: CCCCCOc1cc(N2CCCN(Cc3ccnc4c3c(Cl)cn4CC(=O)N3C[C@@H]4C[C@H]3CO4)C2=O)ccc1OC] |
| 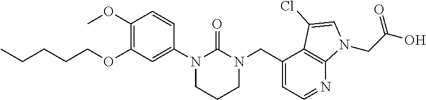 [SMILES: CCCCCOc1cc(N2CCCN(Cc3ccnc4c3c(Cl)cn4CC(=O)O)C2=O)ccc1OC] | 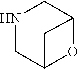 | 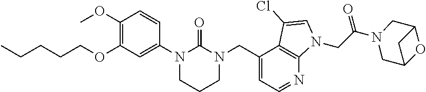 [SMILES: CCCCCOc1cc(N2CCCN(Cc3ccnc4c3c(Cl)cn4CC(=O)N3CC4CC(C3)O4)C2=O)ccc1OC] |
| 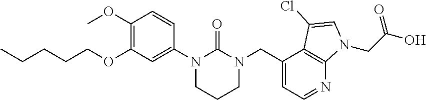 [SMILES: CCCCCOc1cc(N2CCCN(Cc3ccnc4c3c(Cl)cn4CC(=O)O)C2=O)ccc1OC] | 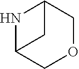 | 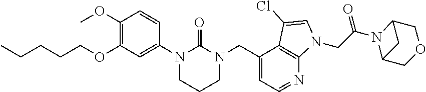 [SMILES: CCCCCOc1cc(N2CCCN(Cc3ccnc4c3c(Cl)cn4CC(=O)N3C4COCC3C4)C2=O)ccc1OC] |
| 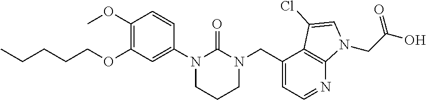 [SMILES: CCCCCOc1cc(N2CCCN(Cc3ccnc4c3c(Cl)cn4CC(=O)O)C2=O)ccc1OC] | 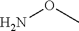 | 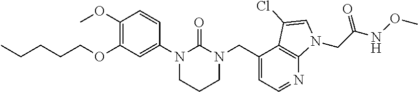 [SMILES: CCCCCOc1cc(N2CCCN(Cc3ccnc4c3c(Cl)cn4CC(=O)NOC)C2=O)ccc1OC] |
| 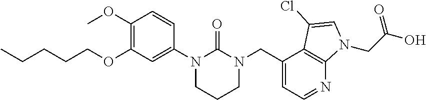 [SMILES: CCCCCOc1cc(N2CCCN(Cc3ccnc4c3c(Cl)cn4CC(=O)O)C2=O)ccc1OC] | 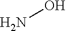 | 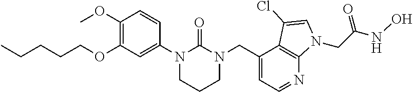 [SMILES: CCCCCOc1cc(N2CCCN(Cc3ccnc4c3c(Cl)cn4CC(=O)NO)C2=O)ccc1OC] |
| 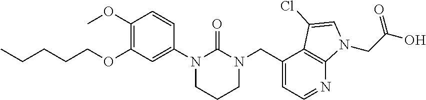 [SMILES: CCCCCOc1cc(N2CCCN(Cc3ccnc4c3c(Cl)cn4CC(=O)O)C2=O)ccc1OC] | 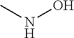 | 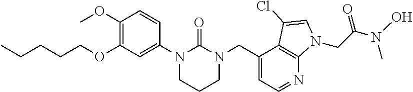 [SMILES: CCCCCOc1cc(N2CCCN(Cc3ccnc4c3c(Cl)cn4CC(=O)N(C)O)C2=O)ccc1OC] |
| 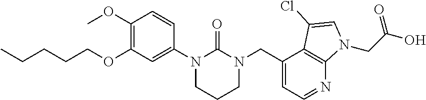 [SMILES: CCCCCOc1cc(N2CCCN(Cc3ccnc4c3c(Cl)cn4CC(=O)O)C2=O)ccc1OC] | 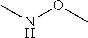 | 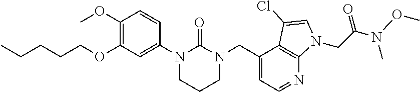 [SMILES: CCCCCOc1cc(N2CCCN(Cc3ccnc4c3c(Cl)cn4CC(=O)N(C)OC)C2=O)ccc1OC] |
| 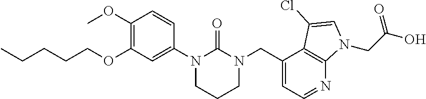 [SMILES: CCCCCOc1cc(N2CCCN(Cc3ccnc4c3c(Cl)cn4CC(=O)O)C2=O)ccc1OC] | 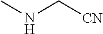 | 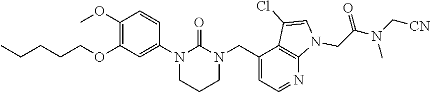 [SMILES: CCCCCOc1cc(N2CCCN(Cc3ccnc4c3c(Cl)cn4CC(=O)N(C)CC#N)C2=O)ccc1OC] |
| 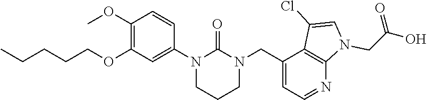 [SMILES: CCCCCOc1cc(N2CCCN(Cc3ccnc4c3c(Cl)cn4CC(=O)O)C2=O)ccc1OC] | 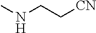 | 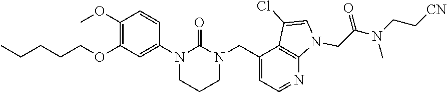 [SMILES: CCCCCOc1cc(N2CCCN(Cc3ccnc4c3c(Cl)cn4CC(=O)N(C)CCC#N)C2=O)ccc1OC] |
| 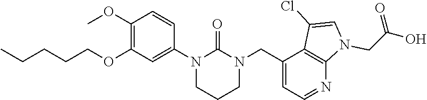 [SMILES: CCCCCOc1cc(N2CCCN(Cc3ccnc4c3c(Cl)cn4CC(=O)O)C2=O)ccc1OC] | 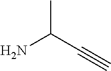 | 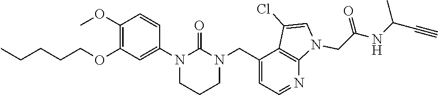 [SMILES: C#CC(C)NC(=O)Cn1cc(Cl)c2c(CN3CCCN(c4ccc(OC)c(OCCCCC)c4)C3=O)ccnc21] |
| 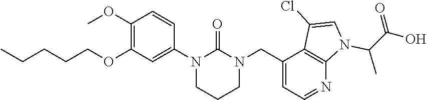 [SMILES: CCCCCOc1cc(N2CCCN(Cc3ccnc4c3c(Cl)cn4C(C)C(=O)O)C2=O)ccc1OC] | 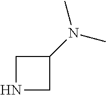 | 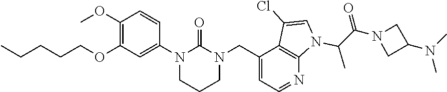 [SMILES: CCCCCOc1cc(N2CCCN(Cc3ccnc4c3c(Cl)cn4C(C)C(=O)N3CC(N(C)C)C3)C2=O)ccc1OC] |
| 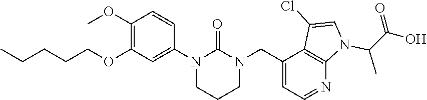 [SMILES: CCCCCOc1cc(N2CCCN(Cc3ccnc4c3c(Cl)cn4C(C)C(=O)O)C2=O)ccc1OC] | 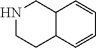 | 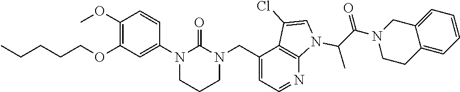 [SMILES: CCCCCOc1cc(N2CCCN(Cc3ccnc4c3c(Cl)cn4C(C)C(=O)N3CCc4ccccc4C3)C2=O)ccc1OC] |
| 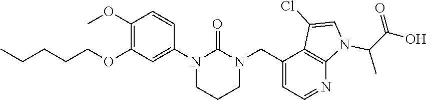 [SMILES: CCCCCOc1cc(N2CCCN(Cc3ccnc4c3c(Cl)cn4C(C)C(=O)O)C2=O)ccc1OC] | 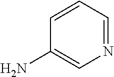 | 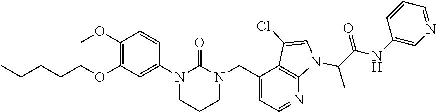 [SMILES: CCCCCOc1cc(N2CCCN(Cc3ccnc4c3c(Cl)cn4C(C)C(=O)Nc3cccnc3)C2=O)ccc1OC] |
| 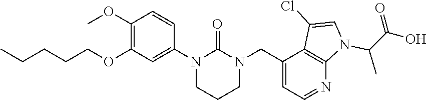 [SMILES: CCCCCOc1cc(N2CCCN(Cc3ccnc4c3c(Cl)cn4C(C)C(=O)O)C2=O)ccc1OC] | 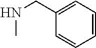 | 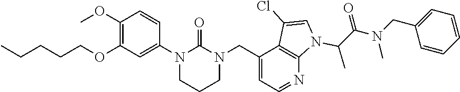 [SMILES: CCCCCOc1cc(N2CCCN(Cc3ccnc4c3c(Cl)cn4C(C)C(=O)N(C)Cc3ccccc3)C2=O)ccc1OC] |
| 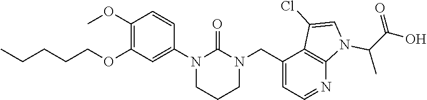 [SMILES: CCCCCOc1cc(N2CCCN(Cc3ccnc4c3c(Cl)cn4C(C)C(=O)O)C2=O)ccc1OC] | 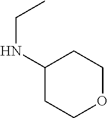 | 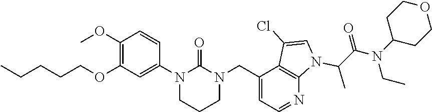 [SMILES: CCCCCOc1cc(N2CCCN(Cc3ccnc4c3c(Cl)cn4C(C)C(=O)N(CC)C3CCOCC3)C2=O)ccc1OC] |
| 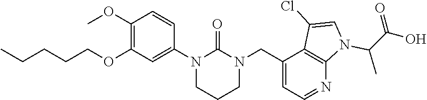 [SMILES: CCCCCOc1cc(N2CCCN(Cc3ccnc4c3c(Cl)cn4C(C)C(=O)O)C2=O)ccc1OC] | H₂N—CN | 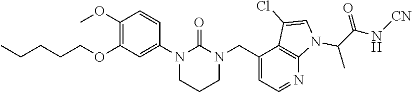 [SMILES: CCCCCOc1cc(N2CCCN(Cc3ccnc4c3c(Cl)cn4C(C)C(=O)NC#N)C2=O)ccc1OC] |
| 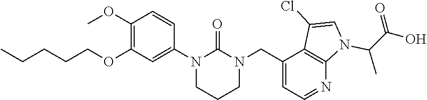 [SMILES: CCCCCOc1cc(N2CCCN(Cc3ccnc4c3c(Cl)cn4C(C)C(=O)O)C2=O)ccc1OC] | 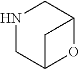 | 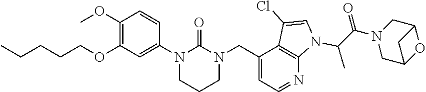 [SMILES: CCCCCOc1cc(N2CCCN(Cc3ccnc4c3c(Cl)cn4C(C)C(=O)N3CC4CC(C3)O4)C2=O)ccc1OC] |
| 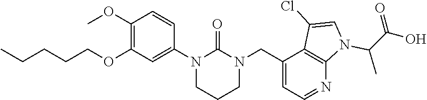 [SMILES: CCCCCOc1cc(N2CCCN(Cc3ccnc4c3c(Cl)cn4C(C)C(=O)O)C2=O)ccc1OC] | 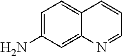 [SMILES: Nc1ccc2cccnc2c1] | 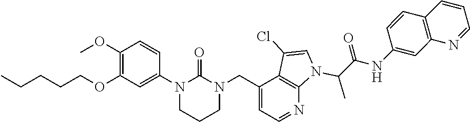 [SMILES: CCCCCOc1cc(N2CCCN(Cc3ccnc4c3c(Cl)cn4C(C)C(=O)Nc3ccc4cccnc4c3)C2=O)ccc1OC] |
| 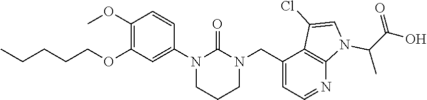 [SMILES: CCCCCOc1cc(N2CCCN(Cc3ccnc4c3c(Cl)cn4C(C)C(=O)O)C2=O)ccc1OC] | 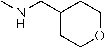 | 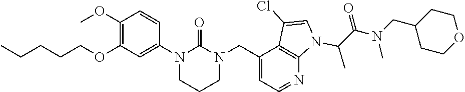 [SMILES: CCCCCOc1cc(N2CCCN(Cc3ccnc4c3c(Cl)cn4C(C)C(=O)N(C)CC3CCOCC3)C2=O)ccc1OC] |
| 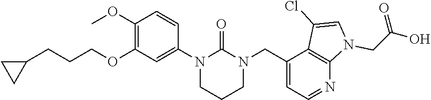 [SMILES: COc1ccc(N2CCCN(Cc3ccnc4c3c(Cl)cn4CC(=O)O)C2=O)cc1OCCCC1CC1] | 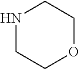 | 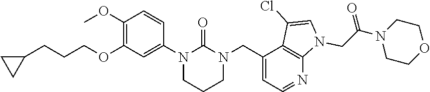 [SMILES: COc1ccc(N2CCCN(Cc3ccnc4c3c(Cl)cn4CC(=O)N3CCOCC3)C2=O)cc1OCCCC1CC1] |
| 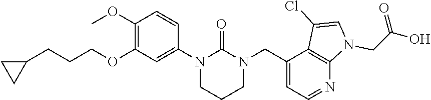 [SMILES: COc1ccc(N2CCCN(Cc3ccnc4c3c(Cl)cn4CC(=O)O)C2=O)cc1OCCCC1CC1] | 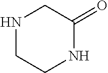 | 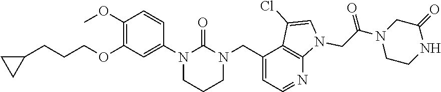 [SMILES: COc1ccc(N2CCCN(Cc3ccnc4c3c(Cl)cn4CC(=O)N3CCNC(=O)C3)C2=O)cc1OCCCC1CC1] |
| 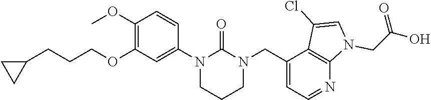 [SMILES: COc1ccc(N2CCCN(Cc3ccnc4c3c(Cl)cn4CC(=O)O)C2=O)cc1OCCCC1CC1] | 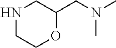 | 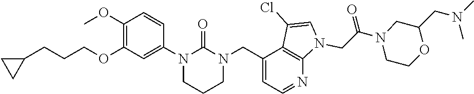 [SMILES: COc1ccc(N2CCCN(Cc3ccnc4c3c(Cl)cn4CC(=O)N3CCOC(CN(C)C)C3)C2=O)cc1OCCCC1CC1] |
| 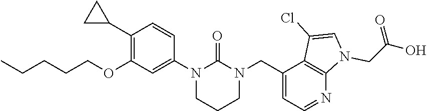 [SMILES: CCCCCOc1cc(N2CCCN(Cc3ccnc4c3c(Cl)cn4CC(=O)O)C2=O)ccc1C1CC1] | 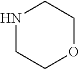 | 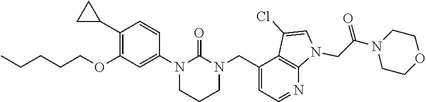 [SMILES: CCCCCOc1cc(N2CCCN(Cc3ccnc4c3c(Cl)cn4CC(=O)N3CCOCC3)C2=O)ccc1C1CC1] |
| 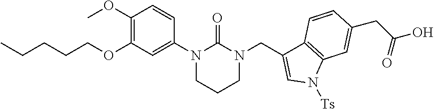 [SMILES: CCCCCOc1cc(N2CCCN(Cc3cn(S(=O)(=O)c4ccc(C)cc4)c4cc(CC(=O)O)ccc34)C2=O)ccc1OC] | 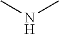 | 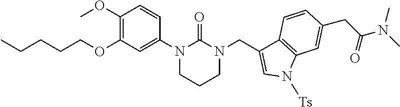 [SMILES: CCCCCOc1cc(N2CCCN(Cc3cn(S(=O)(=O)c4ccc(C)cc4)c4cc(CC(=O)N(C)C)ccc34)C2=O)ccc1OC] |

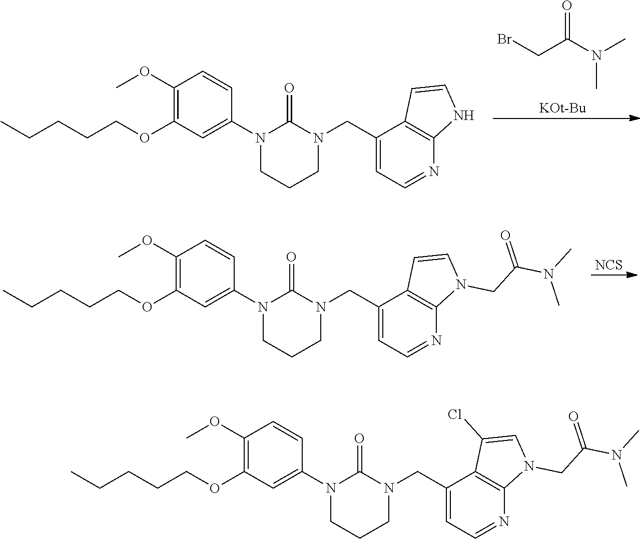

1-((1H-Pyrrolo[2,3-h]pyridin-4-yl)methyl)-3-(4-methoxy-3-(pentyloxy)phenyl)tetrahydropyrimidin-2(1H)-one (2.00 g, 4.73 mmol) is suspended in anhydrous 2-methyltetrahydrofuran (36 mL) at 20° C. and solid potassium tert-butoxide (0.69 g, 6.16 mmol) is added in one portion. The reaction mixture is stirred for 60 minutes before adding a solution of 2-bromo-N,N-dimethylacetamide (0.94 g, 5.68 mmol) in anhydrous 2-methyltetrahydrofuran (4 mL) over 15 minutes. The reaction mixture is stirred at 20° C. for 3 hours before adding water (20 mL). The reaction is extracted with ethyl acetate (3×50 mL). The organic phase is washed with water (3×50 mL), then dried over anhydrous sodium sulfate, filtered, and concentrate in vacuo to afford 2-(4-((3-(4-methoxy-3-(pentyloxy)phenyl)-2-oxotetrahydropyrimidin-1(2H)-yl)methyl)-1H-pyrrolo[2,3-h]pyridin-1l-yl)-N,N-dimethylacetamide (1.83 g).

2-(4-((3-(4-methoxy-3-(pentyloxy)phenyl)-2-oxotetrahydropyrimidin-1(2H)-yl)methyl)-1H-pyrrolo[2,3-b]pyridin-1l-yl)-N,N-dimethylacetamide (0.50 g, 0.98 mmol) is dissolved in N,N-dimethylacetamide (2 mL) at 20° C. and N-chlorosuccinimide (0.14 g, 1.08 mmol) is added in one portion. The reaction mixture is heated to 35° C. and held at 35° C. for 3 hours. tert-Butyl methyl ether (8 mL) is added to the reaction mixture at 35′° C. The mixture is then slowly cooled to −10° C. and held at −10° C. overnight. The solid is collected by vacuum filtration and washed with tert-butyl methyl ether (1 mL). After drying open to air for 60 minutes, the product is further purified by column chromatography over reversed-phase C18 silica gel to afford 2-(3-chloro-4-((3-(4-methoxy-3-(pentyloxy)phenyl)-2-oxotetrahydropyrimidin-1(2H)-yl)methyl)-1H-pyrrolo[2,3-b]pyridin-1l-yl)-N,N-dimethylacetamide (0.31 g).

The following compounds are prepared in a similar manner as described above.

| Substrate | Reactant | Compound |
|---|---|---|
| 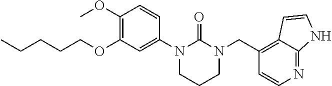 [SMILES: CCCCCOc1cc(N2CCCN(Cc3ccnc4[nH]ccc34)C2=O)ccc1OC] | 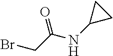 | 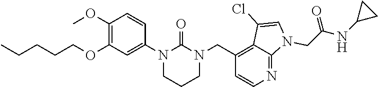 [SMILES: CCCCCOc1cc(N2CCCN(Cc3ccnc4c3c(Cl)cn4CC(=O)NC3CC3)C2=O)ccc1OC] |
| 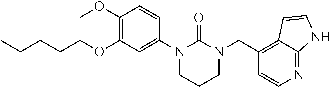 [SMILES: CCCCCOc1cc(N2CCCN(Cc3ccnc4[nH]ccc34)C2=O)ccc1OC] | 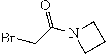 | 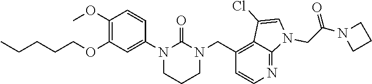 [SMILES: CCCCCOc1cc(N2CCCN(Cc3ccnc4c3c(Cl)cn4CC(=O)N3CCC3)C2=O)ccc1OC] |
| 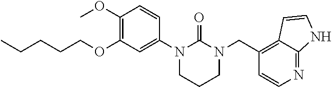 [SMILES: CCCCCOc1cc(N2CCCN(Cc3ccnc4[nH]ccc34)C2=O)ccc1OC] | 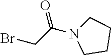 | 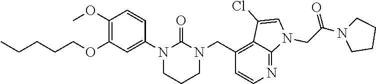 [SMILES: CCCCCOc1cc(N2CCCN(Cc3ccnc4c3c(Cl)cn4CC(=O)N3CCCC3)C2=O)ccc1OC] |
| 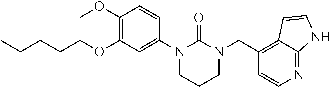 [SMILES: CCCCCOc1cc(N2CCCN(Cc3ccnc4[nH]ccc34)C2=O)ccc1OC] | 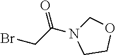 | 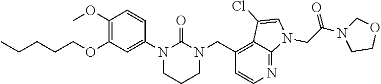 [SMILES: CCCCCOc1cc(N2CCCN(Cc3ccnc4c3c(Cl)cn4CC(=O)N3CCOC3)C2=O)ccc1OC] |
| 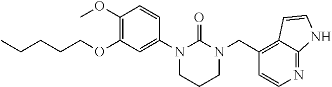 [SMILES: CCCCCOc1cc(N2CCCN(Cc3ccnc4[nH]ccc34)C2=O)ccc1OC] | 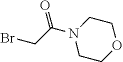 | 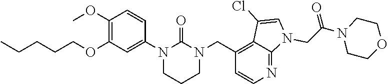 [SMILES: CCCCCOc1cc(N2CCCN(Cc3ccnc4c3c(Cl)cn4CC(=O)N3CCOCC3)C2=O)ccc1OC] |
| 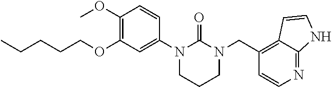 [SMILES: CCCCCOc1cc(N2CCCN(Cc3ccnc4[nH]ccc34)C2=O)ccc1OC] | 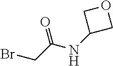 | 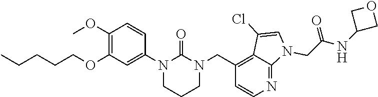 [SMILES: CCCCCOc1cc(N2CCCN(Cc3ccnc4c3c(Cl)cn4CC(=O)NC3COC3)C2=O)ccc1OC] |

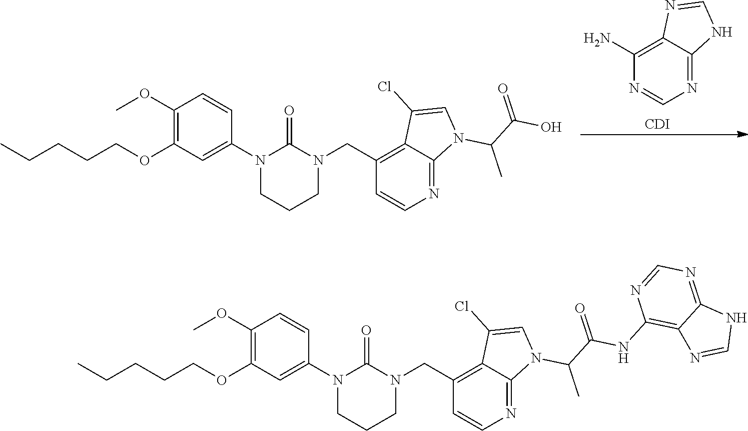

2-(3-chloro-4-((3-(4-methoxy-3-(pentyloxy)phenyl)-2-oxotetrahydropyrimidin-1(2H)-yl)methyl)-1H-pyrrolo[2,3-b]pyridin-1-yl)propanoic acid (150 mg, 0.28 mmol) is dissolved in anhydrous acetonitrile (3 mL) and treated with the slow addition of 1,1′-carbonyldiimidazole (CDI, 54 mg, 0.34 mmol). After complete addition, the reaction is stirred at room temperature for 1 hour. Adenine (42 mg, 0.31 mmol) is added in a single portion. The reaction is stirred at 40° C. overnight. The reaction is diluted with water then extracted with dichloromethane. The combined organic extracts are dried over anhydrous sodium sulfate, filtered, and concentrated in vacuo. Purification by column chromatography over silica gel afforded 2-(3-chloro-4-((3-(4-methoxy-3-(pentyloxy)phenyl)-2-oxotetrahydropyrimidin-1(2H)-yl)methyl)-1H-pyrrolo[2,3-b]pyridin-1-yl)-N-(9H-purin-6-yl)propenamide.

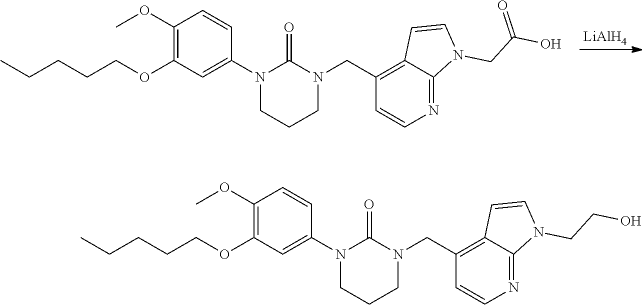

2-(4-((3-(4-methoxy-3-(pentyloxy)phenyl)-2-oxotetrahydropyrimidin-1(2H)-yl)methyl)-1H-pyrrolo[2,3-b]pyridin-1-yl]acetic acid (250 mg, 0.52 mmol) is dissolved in dry tetrahydrofuran (2.5 mL) and cooled to −10° C. The reaction is treated with the dropwise addition of lithium aluminum hydride (2.4M in tetrahydrofuran, 0.22 mL, 0.52 mmol). The reaction is stirred at −10° C. for 2 hours. While at −10° C., the reaction was diluted with diethyl ether (5 mL) then sequentially treated with the careful addition of water (20 uL), 10 wt % aqueous sodium hydroxide solution (20 uL), and more water (60 uL). The resulting suspension is stirred at room temperature for 15 min, then anhydrous sodium sulfate is added to remove any water. The mixture is filtered, and the filter cake is thoroughly washed with ethyl acetate. The filtered solution is concentrated in vacuo. Purification by column chromatography over silica gel afforded 1-((1-(2-hydroxyethyl)-1H-pyrrolo[2,3-b]pyridin-4-yl)methyl)-3-(4-methoxy-3-(pentyloxy)phenyl)tetrahydropyrimidin-2(1H)-one.

The following compounds are prepared in a similar manner as described above.

| Acid or ester | Compound |
|---|---|
| 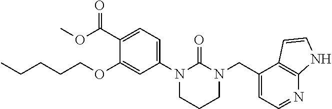 [SMILES: CCCCCOc1cc(N2CCCN(Cc3ccnc4[nH]ccc34)C2=O)ccc1C(=O)OC] | 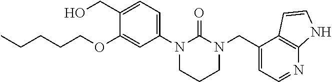 [SMILES: CCCCCOc1cc(N2CCCN(Cc3ccnc4[nH]ccc34)C2=O)ccc1CO] |
| 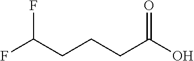 | 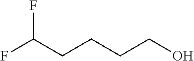 |
| 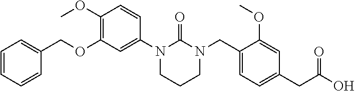 [SMILES: COc1cc(CC(=O)O)ccc1CN1CCCN(c2ccc(OC)c(OCc3ccccc3)c2)C1=O] | 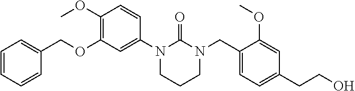 [SMILES: COc1cc(CCO)ccc1CN1CCCN(c2ccc(OC)c(OCc3ccccc3)c2)C1=O] |
| 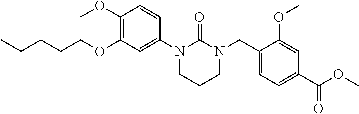 [SMILES: CCCCCOc1cc(N2CCCN(Cc3ccc(C(=O)OC)cc3OC)C2=O)ccc1OC] | 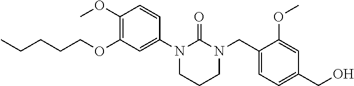 [SMILES: CCCCCOc1cc(N2CCCN(Cc3ccc(CO)cc3OC)C2=O)ccc1OC] |

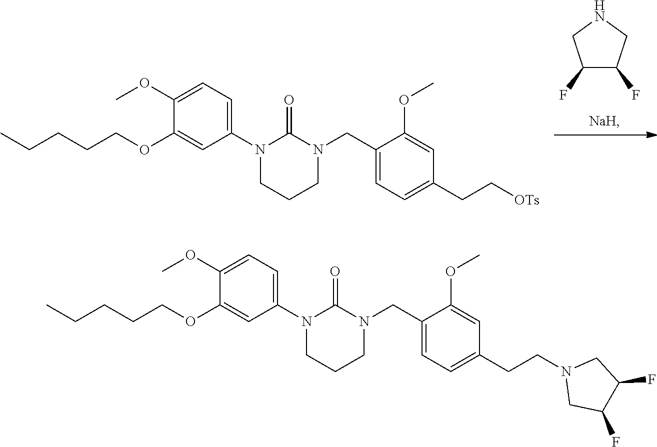 [SMILES: CCCCCOc1cc(N2CCCN(Cc3ccc(CCOS(=O)(=O)c4ccc(C)cc4)cc3OC)C2=O)ccc1OC ; F[C@@H]1CNC[C@@H]1F ; CCCCCOc1cc(N2CCCN(Cc3ccc(CCN4C[C@@H](F)[C@@H](F)C4)cc3OC)C2=O)ccc1OC]

A solution of cis-3,4-difluoropyrrolidine (62 mg, 0.58 mmol) in anhydrous N,N-dimethylformamide (1 mL) is cooled to 0′° C., then treated with sodium hydride (60 wt % in oil, 25 mg, 0.63 mmol). The reaction is stirred for 30 minutes at 0′° C. A solution of 3-methoxy-4-((3-(4-methoxy-3-(pentyloxy)phenyl)-2-oxotetrahydropyrimidin-1(2H)-yl)methyl)phenethyl 4-methylbenzenesulfonate (116 mg, 0.19 mmol) in N,N-dimethylformamide (1 mL) was added at 0° C. The reaction is stirred for 6 hours while slowly warming to room temperature. Water is carefully added, then the reaction is extracted with ethyl acetate. The combined organic layers are washed with brine, dried over anhydrous sodium sulfate, and concentrated in vacuo. The residue is purified by column chromatography over silica gel to afford 1-(4-(2-((3S,4R)-3,4-difluoropyrrolidin-1-yl)ethyl)-2-methoxybenzyl)-3-(4-methoxy-3-(pentyloxy)phenyl)tetrahydropyrimidin-2(1H)-one.

The following compounds are prepared in a similar manner as described above.

| Tosylate | Amine |
|---|---|
| 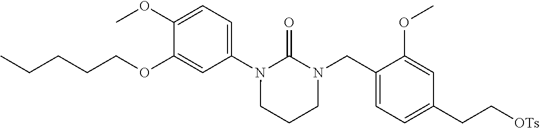 [SMILES: CCCCCOc1cc(N2CCCN(Cc3ccc(CCOS(=O)(=O)c4ccc(C)cc4)cc3OC)C2=O)ccc1OC] | 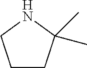 |
| 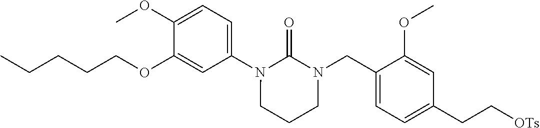 [SMILES: CCCCCOc1cc(N2CCCN(Cc3ccc(CCOS(=O)(=O)c4ccc(C)cc4)cc3OC)C2=O)ccc1OC] | 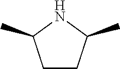 |
| 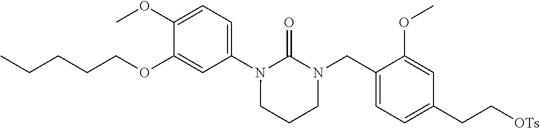 [SMILES: CCCCCOc1cc(N2CCCN(Cc3ccc(CCOS(=O)(=O)c4ccc(C)cc4)cc3OC)C2=O)ccc1OC] | 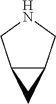 |
| 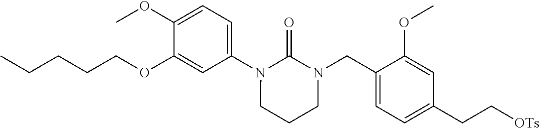 [SMILES: CCCCCOc1cc(N2CCCN(Cc3ccc(CCOS(=O)(=O)c4ccc(C)cc4)cc3OC)C2=O)ccc1OC] | 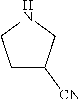 |
| 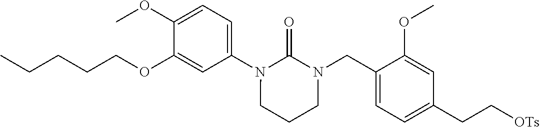 [SMILES: CCCCCOc1cc(N2CCCN(Cc3ccc(CCOS(=O)(=O)c4ccc(C)cc4)cc3OC)C2=O)ccc1OC] | 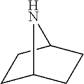 |
| 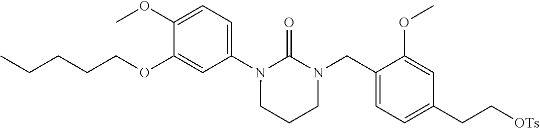 [SMILES: CCCCCOc1cc(N2CCCN(Cc3ccc(CCOS(=O)(=O)c4ccc(C)cc4)cc3OC)C2=O)ccc1OC] | 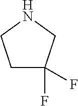 |
| 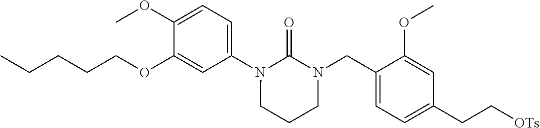 [SMILES: CCCCCOc1cc(N2CCCN(Cc3ccc(CCOS(=O)(=O)c4ccc(C)cc4)cc3OC)C2=O)ccc1OC] | 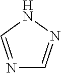 |
| 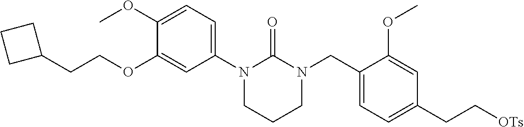 [SMILES: COc1cc(CCOS(=O)(=O)c2ccc(C)cc2)ccc1CN1CCCN(c2ccc(OC)c(OCCC3CCC3)c2)C1=O] | 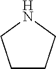 |
| 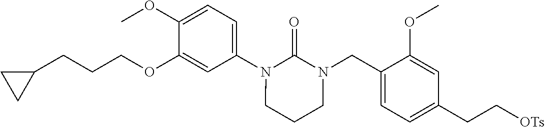 [SMILES: COc1cc(CCOS(=O)(=O)c2ccc(C)cc2)ccc1CN1CCCN(c2ccc(OC)c(OCCCC3CC3)c2)C1=O] | 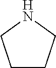 |
| 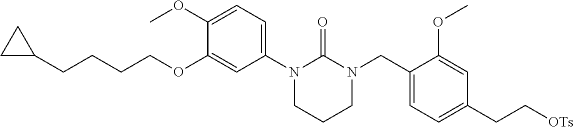 [SMILES: COc1cc(CCOS(=O)(=O)c2ccc(C)cc2)ccc1CN1CCCN(c2ccc(OC)c(OCCCCC3CC3)c2)C1=O] | 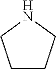 |
| 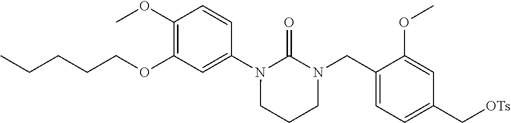 [SMILES: CCCCCOc1cc(N2CCCN(Cc3ccc(COS(=O)(=O)c4ccc(C)cc4)cc3OC)C2=O)ccc1OC] | 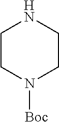 [SMILES: CC(C)(C)OC(=O)N1CCNCC1] |
| 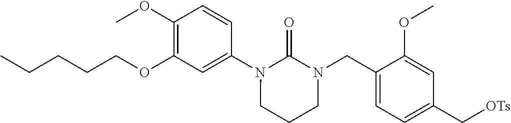 [SMILES: CCCCCOc1cc(N2CCCN(Cc3ccc(COS(=O)(=O)c4ccc(C)cc4)cc3OC)C2=O)ccc1OC] | 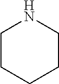 |
| 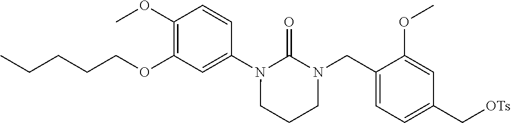 [SMILES: CCCCCOc1cc(N2CCCN(Cc3ccc(COS(=O)(=O)c4ccc(C)cc4)cc3OC)C2=O)ccc1OC] | 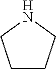 |
| 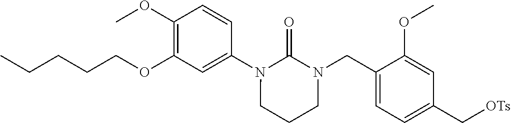 [SMILES: CCCCCOc1cc(N2CCCN(Cc3ccc(COS(=O)(=O)c4ccc(C)cc4)cc3OC)C2=O)ccc1OC] | 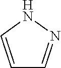 |
| 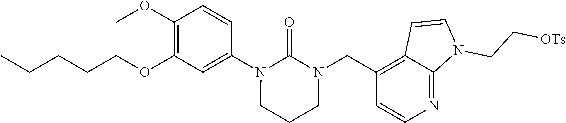 [SMILES: CCCCCOc1cc(N2CCCN(Cc3ccnc4c3ccn4CCOS(=O)(=O)c3ccc(C)cc3)C2=O)ccc1OC] | 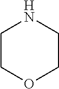 |
| 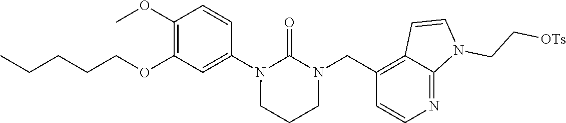 [SMILES: CCCCCOc1cc(N2CCCN(Cc3ccnc4c3ccn4CCOS(=O)(=O)c3ccc(C)cc3)C2=O)ccc1OC] | 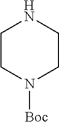 [SMILES: CC(C)(C)OC(=O)N1CCNCC1] |
| 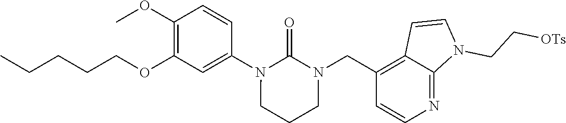 [SMILES: CCCCCOc1cc(N2CCCN(Cc3ccnc4c3ccn4CCOS(=O)(=O)c3ccc(C)cc3)C2=O)ccc1OC] | 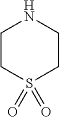 |
| 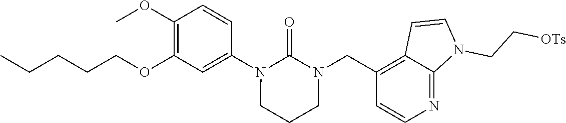 [SMILES: CCCCCOc1cc(N2CCCN(Cc3ccnc4c3ccn4CCOS(=O)(=O)c3ccc(C)cc3)C2=O)ccc1OC] | 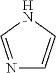 |
| 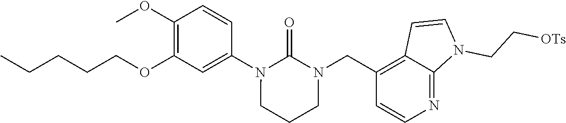 [SMILES: CCCCCOc1cc(N2CCCN(Cc3ccnc4c3ccn4CCOS(=O)(=O)c3ccc(C)cc3)C2=O)ccc1OC] | 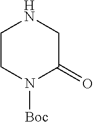 [SMILES: CC(C)(C)OC(=O)N1CCNCC1=O] |
| 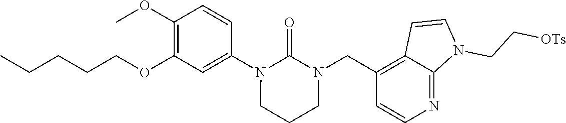 [SMILES: CCCCCOc1cc(N2CCCN(Cc3ccnc4c3ccn4CCOS(=O)(=O)c3ccc(C)cc3)C2=O)ccc1OC] | 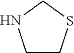 |
| 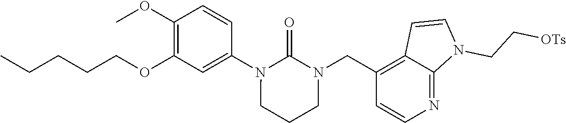 [SMILES: CCCCCOc1cc(N2CCCN(Cc3ccnc4c3ccn4CCOS(=O)(=O)c3ccc(C)cc3)C2=O)ccc1OC] | 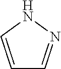 |
| 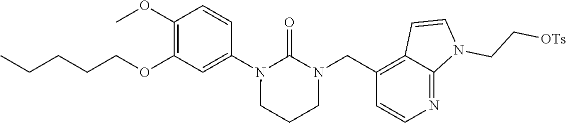 [SMILES: CCCCCOc1cc(N2CCCN(Cc3ccnc4c3ccn4CCOS(=O)(=O)c3ccc(C)cc3)C2=O)ccc1OC] | 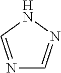 |
| 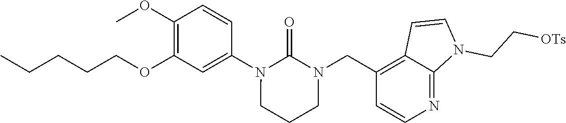 [SMILES: CCCCCOc1cc(N2CCCN(Cc3ccnc4c3ccn4CCOS(=O)(=O)c3ccc(C)cc3)C2=O)ccc1OC] | 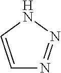 |
| 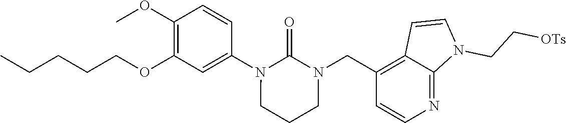 [SMILES: CCCCCOc1cc(N2CCCN(Cc3ccnc4c3ccn4CCOS(=O)(=O)c3ccc(C)cc3)C2=O)ccc1OC] | 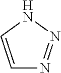 |
| 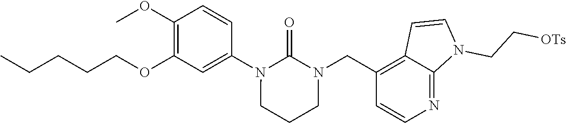 [SMILES: CCCCCOc1cc(N2CCCN(Cc3ccnc4c3ccn4CCOS(=O)(=O)c3ccc(C)cc3)C2=O)ccc1OC] | 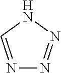 |
| 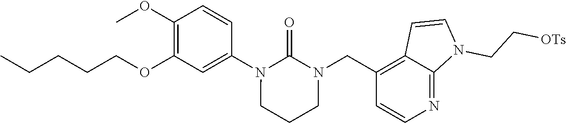 [SMILES: CCCCCOc1cc(N2CCCN(Cc3ccnc4c3ccn4CCOS(=O)(=O)c3ccc(C)cc3)C2=O)ccc1OC] | 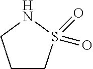 |
| 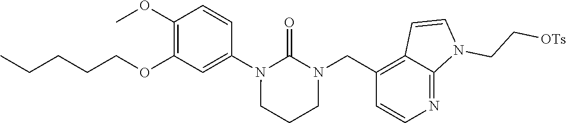 [SMILES: CCCCCOc1cc(N2CCCN(Cc3ccnc4c3ccn4CCOS(=O)(=O)c3ccc(C)cc3)C2=O)ccc1OC] | 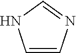 |
| 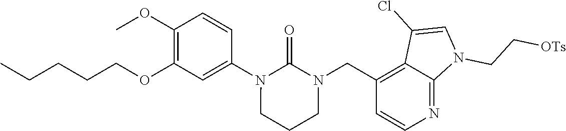 [SMILES: CCCCCOc1cc(N2CCCN(Cc3ccnc4c3c(Cl)cn4CCOS(=O)(=O)c3ccc(C)cc3)C2=O)ccc1OC] | 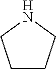 |

Compound

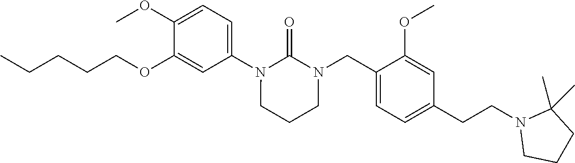 [SMILES: CCCCCOc1cc(N2CCCN(Cc3ccc(CCN4CCCC4(C)C)cc3OC)C2=O)ccc1OC]

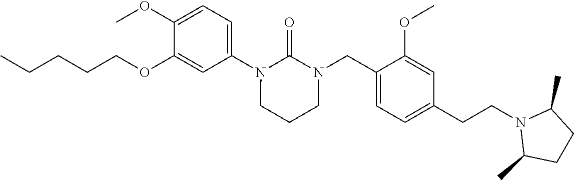 [SMILES: CCCCCOc1cc(N2CCCN(Cc3ccc(CCN4[C@H](C)CC[C@@H]4C)cc3OC)C2=O)ccc1OC]

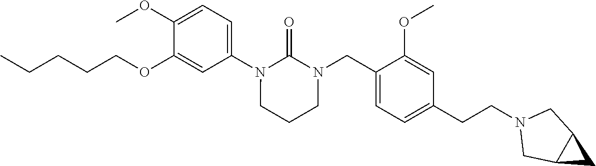 [SMILES: CCCCCOc1cc(N2CCCN(Cc3ccc(CCN4C[C@H]5C[C@H]5C4)cc3OC)C2=O)ccc1OC]

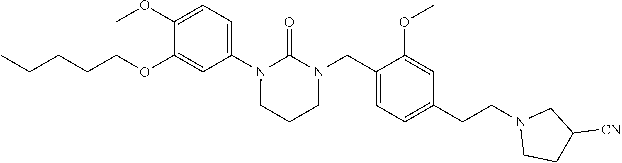 [SMILES: CCCCCOc1cc(N2CCCN(Cc3ccc(CCN4CCC(C#N)C4)cc3OC)C2=O)ccc1OC]

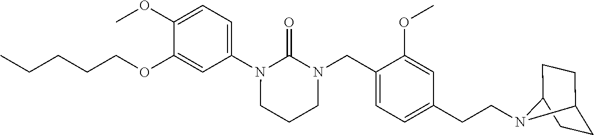 [SMILES: CCCCCOc1cc(N2CCCN(Cc3ccc(CCN4C5CCC4CC5)cc3OC)C2=O)ccc1OC]

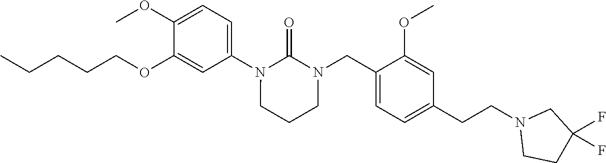 [SMILES: CCCCCOc1cc(N2CCCN(Cc3ccc(CCN4CCC(F)(F)C4)cc3OC)C2=O)ccc1OC]

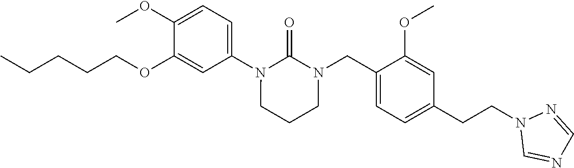 [SMILES: CCCCCOc1cc(N2CCCN(Cc3ccc(CCn4cncn4)cc3OC)C2=O)ccc1OC]

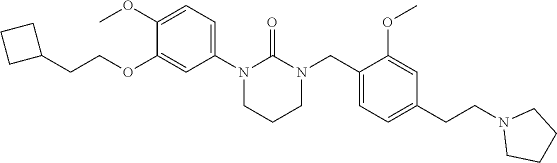 [SMILES: COc1cc(CCN2CCCC2)ccc1CN1CCCN(c2ccc(OC)c(OCCC3CCC3)c2)C1=O]

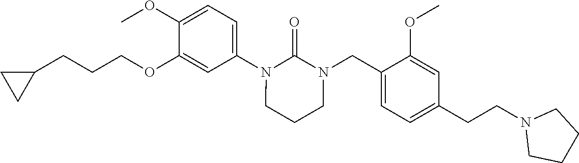 [SMILES: COc1cc(CCN2CCCC2)ccc1CN1CCCN(c2ccc(OC)c(OCCCC3CC3)c2)C1=O]

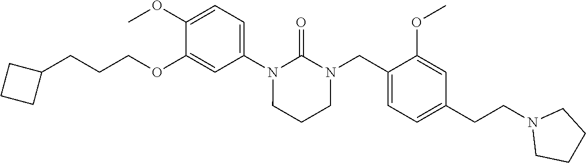 [SMILES: COc1cc(CCN2CCCC2)ccc1CN1CCCN(c2ccc(OC)c(OCCCC3CCC3)c2)C1=O]

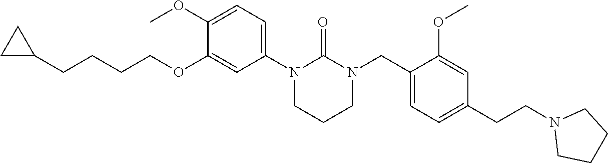 [SMILES: COc1cc(CCN2CCCC2)ccc1CN1CCCN(c2ccc(OC)c(OCCCCC3CC3)c2)C1=O]

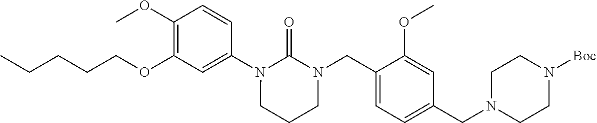 [SMILES: CCCCCOc1cc(N2CCCN(Cc3ccc(CN4CCN(C(=O)OC(C)(C)C)CC4)cc3OC)C2=O)ccc1OC]

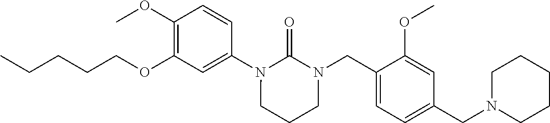 [SMILES: CCCCCOc1cc(N2CCCN(Cc3ccc(CN4CCCCC4)cc3OC)C2=O)ccc1OC]

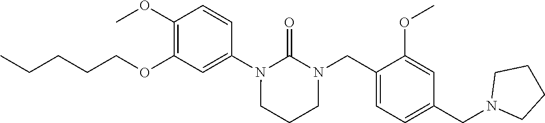 [SMILES: CCCCCOc1cc(N2CCCN(Cc3ccc(CN4CCCC4)cc3OC)C2=O)ccc1OC]

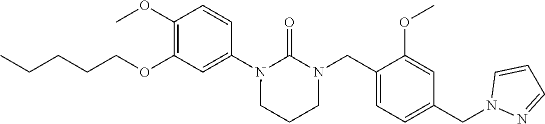 [SMILES: CCCCCOc1cc(N2CCCN(Cc3ccc(Cn4cccn4)cc3OC)C2=O)ccc1OC]

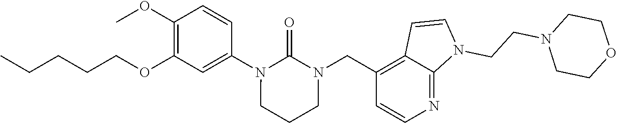 [SMILES: CCCCCOc1cc(N2CCCN(Cc3ccnc4c3ccn4CCN3CCOCC3)C2=O)ccc1OC]

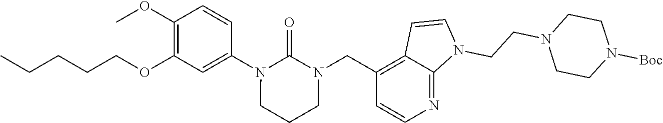 [SMILES: CCCCCOc1cc(N2CCCN(Cc3ccnc4c3ccn4CCN3CCN(C(=O)OC(C)(C)C)CC3)C2=O)ccc1OC]

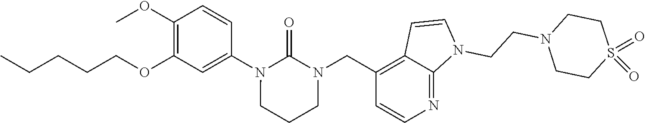 [SMILES: CCCCCOc1cc(N2CCCN(Cc3ccnc4c3ccn4CCN3CCS(=O)(=O)CC3)C2=O)ccc1OC]

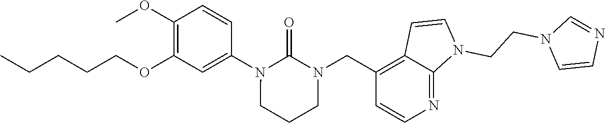 [SMILES: CCCCCOc1cc(N2CCCN(Cc3ccnc4c3ccn4CCn3ccnc3)C2=O)ccc1OC]

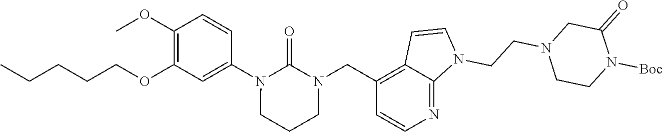 [SMILES: CCCCCOc1cc(N2CCCN(Cc3ccnc4c3ccn4CCN3CCN(C(=O)OC(C)(C)C)C(=O)C3)C2=O)ccc1OC]

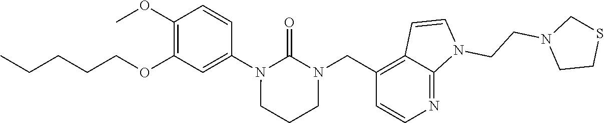 [SMILES: CCCCCOc1cc(N2CCCN(Cc3ccnc4c3ccn4CCN3CCSC3)C2=O)ccc1OC]

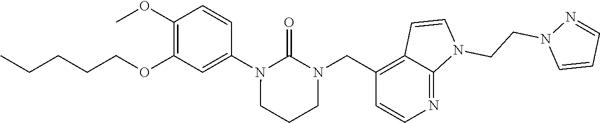 [SMILES: CCCCCOc1cc(N2CCCN(Cc3ccnc4c3ccn4CCn3cccn3)C2=O)ccc1OC]

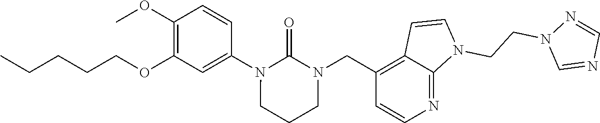 [SMILES: CCCCCOc1cc(N2CCCN(Cc3ccnc4c3ccn4CCn3cncn3)C2=O)ccc1OC]

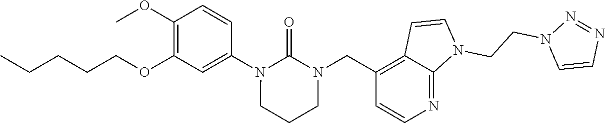 [SMILES: CCCCCOc1cc(N2CCCN(Cc3ccnc4c3ccn4CCn3ccnn3)C2=O)ccc1OC]

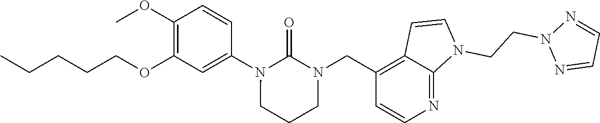 [SMILES: CCCCCOc1cc(N2CCCN(Cc3ccnc4c3ccn4CCn3nccn3)C2=O)ccc1OC]

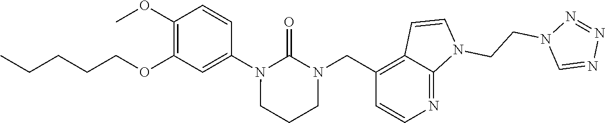 [SMILES: CCCCCOc1cc(N2CCCN(Cc3ccnc4c3ccn4CCn3cnnn3)C2=O)ccc1OC]

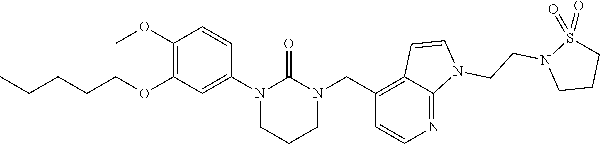 [SMILES: CCCCCOc1cc(N2CCCN(Cc3ccnc4c3ccn4CCN3CCCS3(=O)=O)C2=O)ccc1OC]

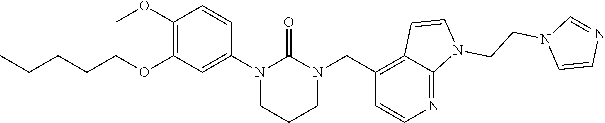 [SMILES: CCCCCOc1cc(N2CCCN(Cc3ccnc4c3ccn4CCn3ccnc3)C2=O)ccc1OC]

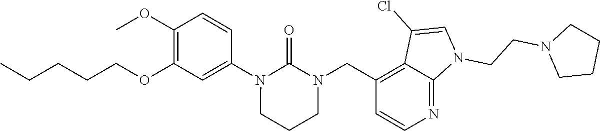 [SMILES: CCCCCOc1cc(N2CCCN(Cc3ccnc4c3c(Cl)cn4CCN3CCCC3)C2=O)ccc1OC]

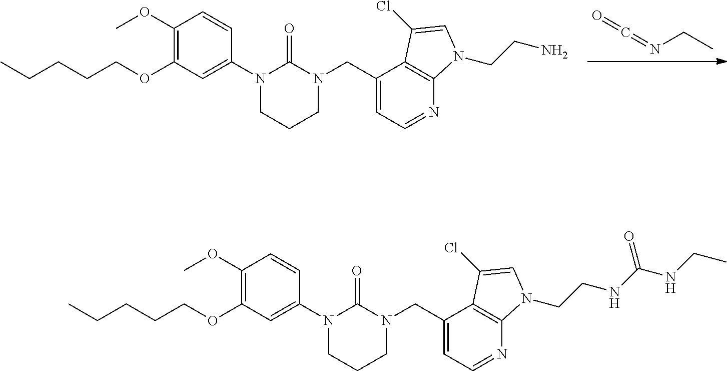

A solution of 1-((1-(2-aminoethyl)-3-chloro-1H-pyrrolo[2,3-b]pyridin-4-yl)methyl)-3-(4-methoxy-3-(pentyloxy)phenyl)tetrahydropyrimidin-2(1H)-one (125 mg, 0.25 mmol) in anhydrous tetrahydrofuran (10 mL) is treated with neat ethyl isocyanate (22 uL, 0.28 mmol). The reaction is stirred at room temperature overnight. The reaction is concentrated in vacuo, and the residue is purified by column chromatography over silica gel to afford 1-(2-(3-chloro-4-((3-(4-methoxy-3-(pentyloxy)phenyl)-2-oxotetrahydropyrimidin-1(2H)-yl)methyl)-1H-pyrrolo[2,3-b]pyridin-1-yl)ethyl)-3-ethylurea.

The following compounds are prepared in a similar manner as described above.

| Starting material | Isocyanate |
|---|---|
| 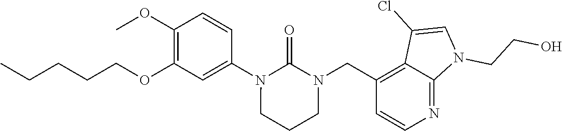 [SMILES: CCCCCOc1cc(N2CCCN(Cc3ccnc4c3c(Cl)cn4CCO)C2=O)ccc1OC] | 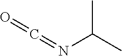 |

Compound

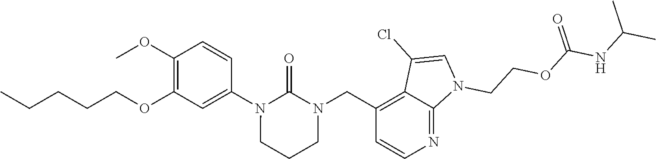 [SMILES: CCCCCOc1cc(N2CCCN(Cc3ccnc4c3c(Cl)cn4CCOC(=O)NC(C)C)C2=O)ccc1OC]

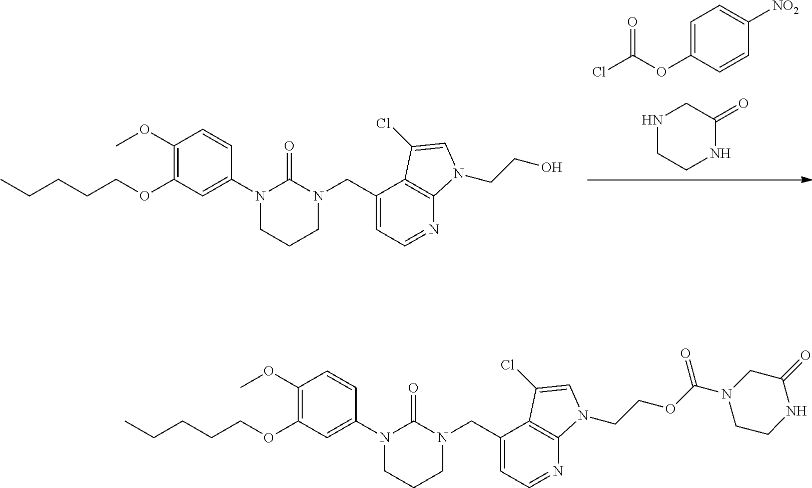

Piperazin-2-one (15 mg, 0.15 mmol) is dissolved in anhydrous tetrahydrofuran (1 mL) and cooled to 0° C. 4-nitrophenyl chloroformate (30 mg, 0.15 mmol) is added in a single portion. The reaction is warmed to room temperature and stirred for 30 minutes. The reaction is then cooled to 0° C. again. 1-((3-chloro-1-(2-hydroxyethyl)-1H-pyrrolo[2,3-b]pyridin-4-yl)methyl)-3-(4-methoxy-3-(pentyloxy)phenyl)tetrahydropyrimidin-2(1H)-one (75 mg, 0.15 mmol) is added, followed by triethylamine (52 uL, 0.38 mmol). The reaction is warmed to room temperature and stirred overnight. Water is added, and the reaction is extracted with dichloromethane. The combined organic extracts are washed with saturated aqueous ammonium chloride solution, then with water, and brine. The organic phase is dried over anhydrous sodium sulfate, filtered, and concentrated in vacuo. Purification by column chromatography over silica gel afforded 2-(3-chloro-4-((3-(4-methoxy-3-(pentyloxy)phenyl)-2-oxotetrahydropyrimidin-1(2H)-yl)methyl)-1H-pyrrolo[2,3-b]pyridin-1-yl)ethyl 3-oxopiperazine-1-carboxylate.

The following compounds are prepared in a similar manner as described above.

| | Amine |
|---|---|
| | or |
| Alcohol or amine | alcohol |
| 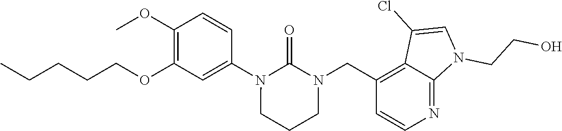 [SMILES: CCCCCOc1cc(N2CCCN(Cc3ccnc4c3c(Cl)cn4CCO)C2=O)ccc1OC] | 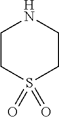 |
| 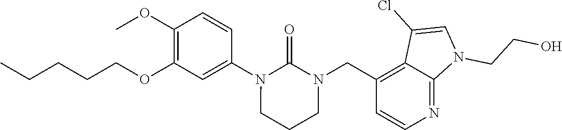 [SMILES: CCCCCOc1cc(N2CCCN(Cc3ccnc4c3c(Cl)cn4CCO)C2=O)ccc1OC] | 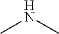 |
| 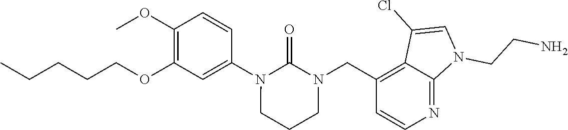 [SMILES: CCCCCOc1cc(N2CCCN(Cc3ccnc4c3c(Cl)cn4CCN)C2=O)ccc1OC] | 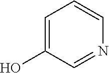 |

Compound

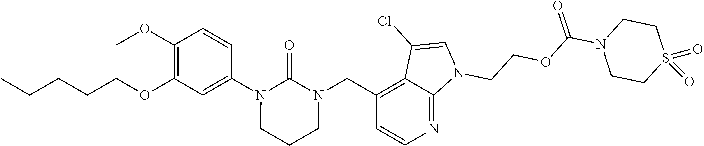 [SMILES: CCCCCOc1cc(N2CCCN(Cc3ccnc4c3c(Cl)cn4CCOC(=O)N3CCS(=O)(=O)CC3)C2=O)ccc1OC]

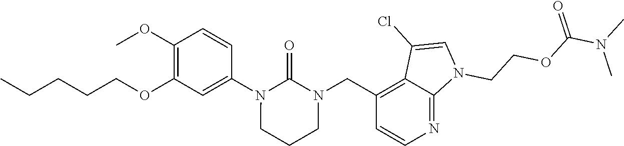 [SMILES: CCCCCOc1cc(N2CCCN(Cc3ccnc4c3c(Cl)cn4CCOC(=O)N(C)C)C2=O)ccc1OC]

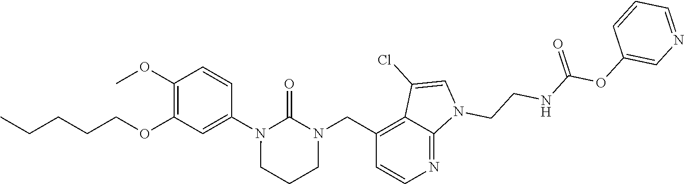 [SMILES: CCCCCOc1cc(N2CCCN(Cc3ccnc4c3c(Cl)cn4CCNC(=O)Oc3cccnc3)C2=O)ccc1OC]

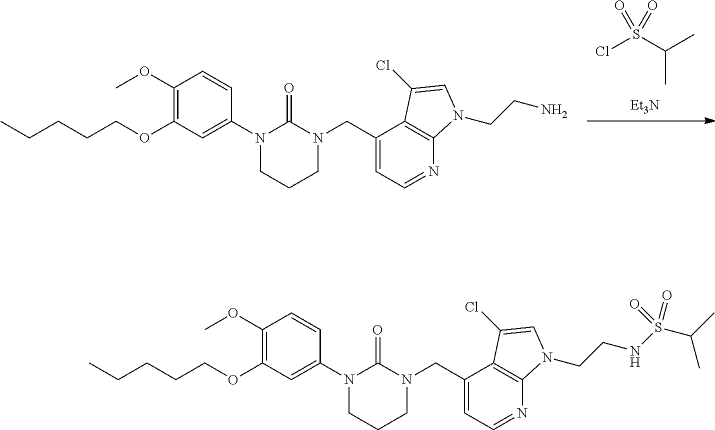

A solution of 1-((1-(2-aminoethyl)-3-chloro-1H-pyrrolo[2,3-b]pyridin-4-yl)methyl)-3-(4-methoxy-3-(pentyloxy)phenyl)tetrahydropyrimidin-2(1H)-one (90 mg, 0.18 mmol) in anhydrous dichloromethane is cooled to 0° C. before adding triethylamine (37 uL, 0.27 mmol) then isopropylsulfonyl chloride (22 uL, 0.20 mmol). The reaction is stirred at 0° C. for 3 hours. Saturated aqueous ammonium chloride is added, then the reaction mixture is extracted with ethyl acetate. The combined organic extracts are dried over anhydrous magnesium sulfate, filtered, and concentrated in vacuo. Purification by column chromatography over silica gel afforded (N-(2-(3-chloro-4-((3-(4-methoxy-3-(pentyloxy)phenyl)-2-oxotetrahydropyrimidin-1(2H)-yl)methyl)-1H-pyrrolo[2,3-b]pyridin-1l-yl)ethyl)propane-2-sulfonamide).

The following compounds are prepared in a similar manner as described above:

| | Sulfonyl |
|---|---|
| Amine | chloride |
| 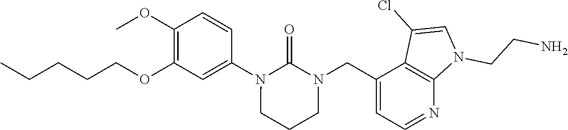 [SMILES: CCCCCOc1cc(N2CCCN(Cc3ccnc4c3c(Cl)cn4CCN)C2=O)ccc1OC] | 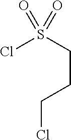 |
| 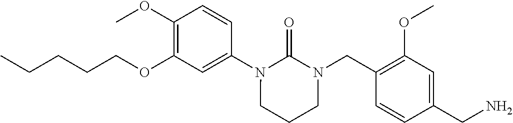 [SMILES: CCCCCOc1cc(N2CCCN(Cc3ccc(CN)cc3OC)C2=O)ccc1OC] | 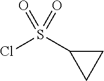 |
| 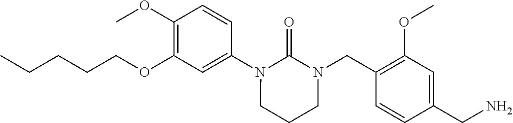 [SMILES: CCCCCOc1cc(N2CCCN(Cc3ccc(CN)cc3OC)C2=O)ccc1OC] | 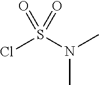 |
| 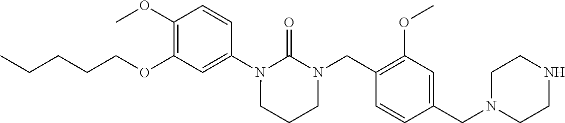 [SMILES: CCCCCOc1cc(N2CCCN(Cc3ccc(CN4CCNCC4)cc3OC)C2=O)ccc1OC] | 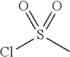 |
| 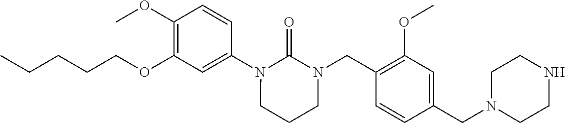 [SMILES: CCCCCOc1cc(N2CCCN(Cc3ccc(CN4CCNCC4)cc3OC)C2=O)ccc1OC] | 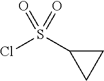 |
| 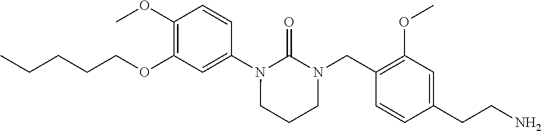 [SMILES: CCCCCOc1cc(N2CCCN(Cc3ccc(CCN)cc3OC)C2=O)ccc1OC] | 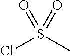 |
| 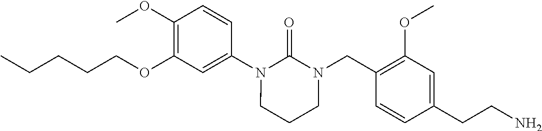 [SMILES: CCCCCOc1cc(N2CCCN(Cc3ccc(CCN)cc3OC)C2=O)ccc1OC] | 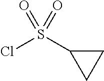 |
| 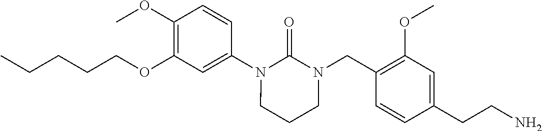 [SMILES: CCCCCOc1cc(N2CCCN(Cc3ccc(CCN)cc3OC)C2=O)ccc1OC] | 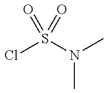 |
| 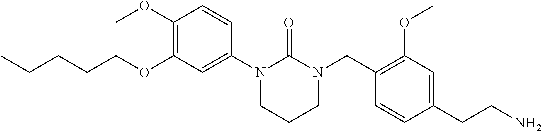 [SMILES: CCCCCOc1cc(N2CCCN(Cc3ccc(CCN)cc3OC)C2=O)ccc1OC] | 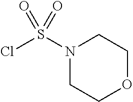 |
| 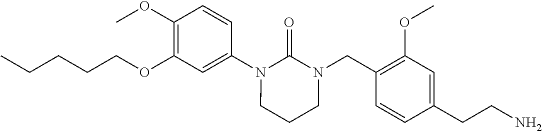 [SMILES: CCCCCOc1cc(N2CCCN(Cc3ccc(CCN)cc3OC)C2=O)ccc1OC] | 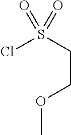 |
| 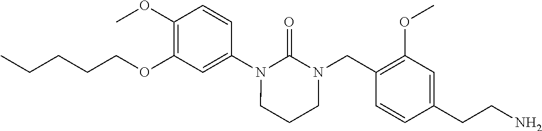 [SMILES: CCCCCOc1cc(N2CCCN(Cc3ccc(CCN)cc3OC)C2=O)ccc1OC] | 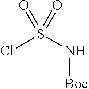 [SMILES: CC(C)(C)OC(=O)NS(=O)(=O)Cl] |

Compound

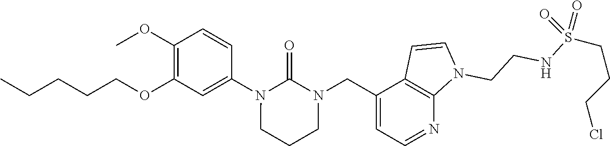 [SMILES: CCCCCOc1cc(N2CCCN(Cc3ccnc4c3ccn4CCNS(=O)(=O)CCCCl)C2=O)ccc1OC]

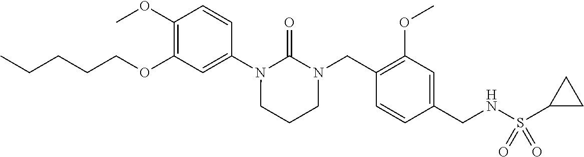 [SMILES: CCCCCOc1cc(N2CCCN(Cc3ccc(CNS(=O)(=O)C4CC4)cc3OC)C2=O)ccc1OC]

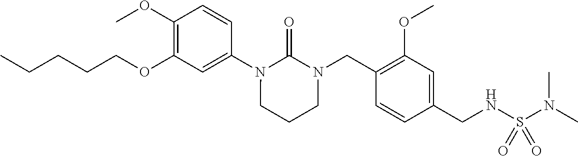 [SMILES: CCCCCOc1cc(N2CCCN(Cc3ccc(CNS(=O)(=O)N(C)C)cc3OC)C2=O)ccc1OC]

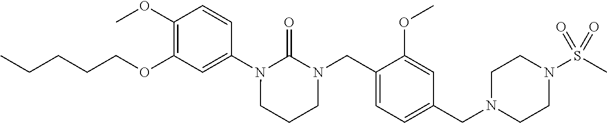 [SMILES: CCCCCOc1cc(N2CCCN(Cc3ccc(CN4CCN(S(C)(=O)=O)CC4)cc3OC)C2=O)ccc1OC]

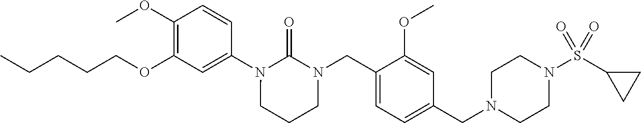 [SMILES: CCCCCOc1cc(N2CCCN(Cc3ccc(CN4CCN(S(=O)(=O)C5CC5)CC4)cc3OC)C2=O)ccc1OC]

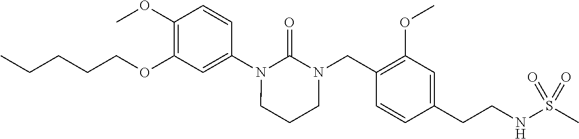 [SMILES: CCCCCOc1cc(N2CCCN(Cc3ccc(CCNS(C)(=O)=O)cc3OC)C2=O)ccc1OC]

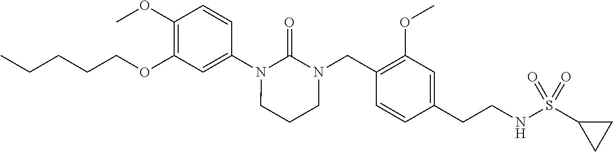 [SMILES: CCCCCOc1cc(N2CCCN(Cc3ccc(CCNS(=O)(=O)C4CC4)cc3OC)C2=O)ccc1OC]

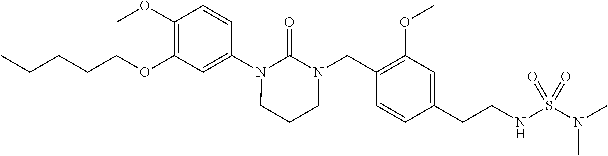 [SMILES: CCCCCOc1cc(N2CCCN(Cc3ccc(CCNS(=O)(=O)N(C)C)cc3OC)C2=O)ccc1OC]

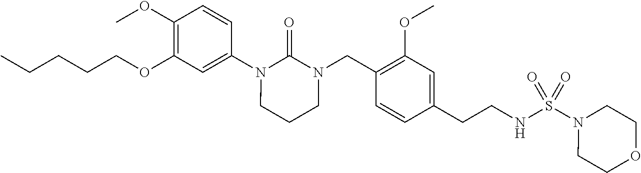 [SMILES: CCCCCOc1cc(N2CCCN(Cc3ccc(CCNS(=O)(=O)N4CCOCC4)cc3OC)C2=O)ccc1OC]

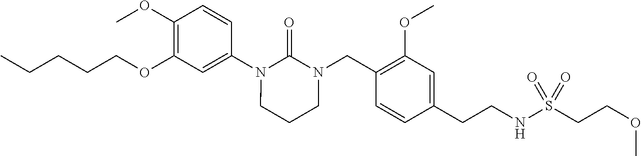 [SMILES: CCCCCOc1cc(N2CCCN(Cc3ccc(CCNS(=O)(=O)CCOC)cc3OC)C2=O)ccc1OC]

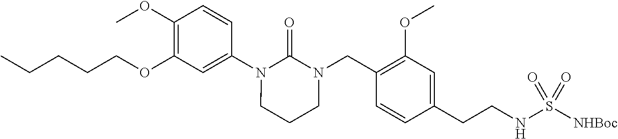 [SMILES: CCCCCOc1cc(N2CCCN(Cc3ccc(CCNS(=O)(=O)NC(=O)OC(C)(C)C)cc3OC)C2=O)ccc1OC]

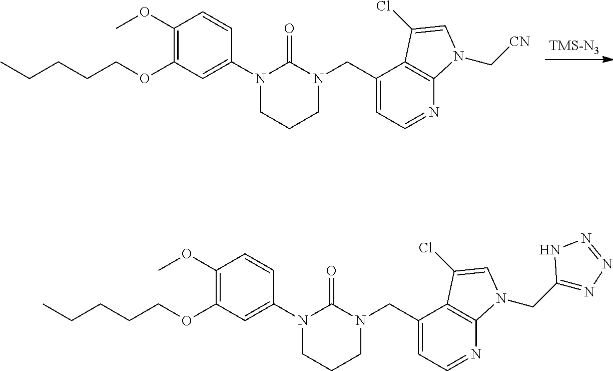 [SMILES: CCCCCOc1cc(N2CCCN(Cc3ccnc4c3c(Cl)cn4CC#N)C2=O)ccc1OC ; "TMS-N3" ; CCCCCOc1cc(N2CCCN(Cc3ccnc4c3c(Cl)cn4Cc3nnn[nH]3)C2=O)ccc1OC]

2-(3-chloro-4-((3-(4-methoxy-3-(pentyloxy)phenyl)-2-oxotetrahydropyrimidin-1(2H)-yl)methyl)-1H-pyrrolo[2,3-b]pyridin-1-yl)acetonitrile (300 mg, 0.60 mmol) is dissolved in anhydrous N,N-dimethylformamide (3 mL). Ammonium chloride (65 mg, 1.21 mmol) is added, followed by azidotrimethylsilane (161 uL, 1.21 mmol). The reaction is stirred overnight at 80′° C. After cooling to room temperature, the reaction is quenched with water, then extracted with dichloromethane. The combined organic extracts are dried over anhydrous sodium sulfate, filtered, and concentrated in vacuo. Purification by column chromatography over reversed-phase C18 silica gel afforded 1-((1-((1H-tetrazol-5-yl)methyl)-3-chloro-1H-pyrrolo[2,3-b]pyridin-4-yl)methyl)-3-(4-methoxy-3-(pentyloxy)phenyl)tetrahydropyrimidin-2(1H)-one.

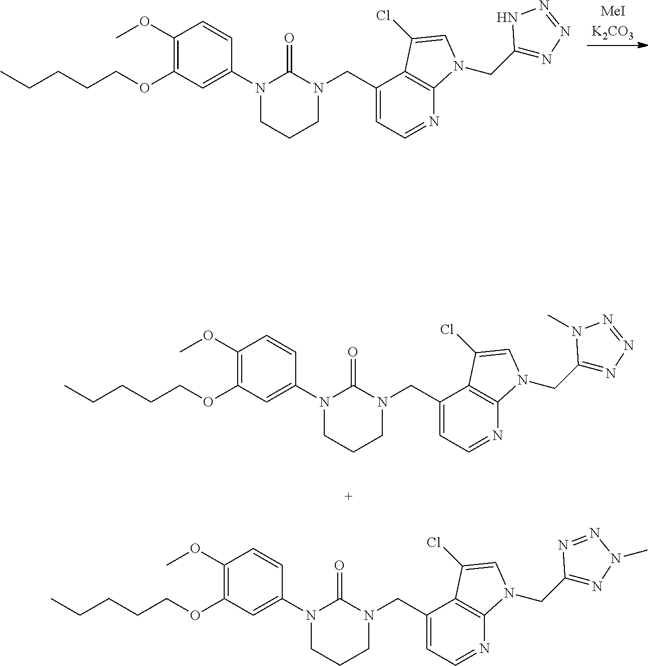 [SMILES: "MeIK2CO3" ; CCCCCOc1cc(N2CCCN(Cc3ccnc4c3c(Cl)cn4Cc3nnn[nH]3)C2=O)ccc1OC ; CCCCCOc1cc(N2CCCN(Cc3ccnc4c3c(Cl)cn4Cc3nnnn3C)C2=O)ccc1OC ; CCCCCOc1cc(N2CCCN(Cc3ccnc4c3c(Cl)cn4Cc3nnn(C)n3)C2=O)ccc1OC]

A solution of 1-((1-((1H-tetrazol-5-yl)methyl)-3-chloro-1H-pyrrolo[2,3-b]pyridin-4-yl)methyl)-3-(4-methoxy-3-(pentyloxy)phenyl)tetrahydropyrimidin-2(1H)-one (200 mg, 0.37 mmol) in anhydrous acetone (2 mL) is treated with anhydrous potassium carbonate (77 mg, 0.56 mmol) and iodomethane (35 uL, 0.56 mmol). The reaction is heated to 60° C. and stirred overnight at this temperature. After cooling to room temperature, the solids are removed by filtration through CELITE®. The filter cake is washed thoroughly with acetone. The filtered solution is concentrated in vacuo. Purification by column chromatography over reversed-phase C18 silica gel afforded 1-((3-chloro-1-((1-methyl-1H-tetrazol-5-yl)methyl)-1H-pyrrolo[2,3-b]pyridin-4-yl)methyl)-3-(4-methoxy-3-(pentyloxy)phenyl)tetrahydropyrimidin-2(1H)-one & 1-((3-chloro-1-((2-methyl-2H-tetrazol-5-yl)methyl)-1H-pyrrolo[2,3-b]pyridin-4-yl)methyl)-3-(4-methoxy-3-(pentyloxy)phenyl)tetrahydropyrimidin-2(1H)-one.

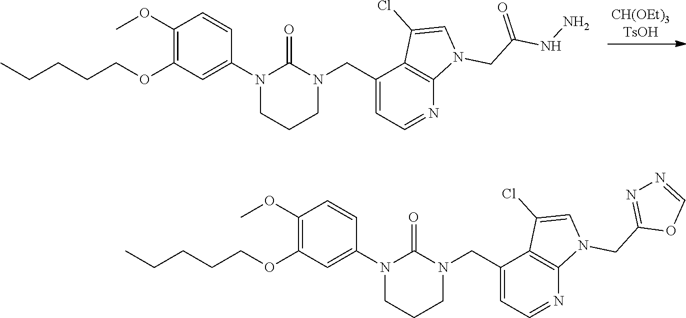 [SMILES: CCCCCOc1cc(N2CCCN(Cc3ccnc4c3c(Cl)cn4CC(=O)NN)C2=O)ccc1OC ; CCCCCOc1cc(N2CCCN(Cc3ccnc4c3c(Cl)cn4Cc3nnco3)C2=O)ccc1OC]

A mixture of 2-(3-chloro-4-((3-(4-methoxy-3-(pentyloxy)phenyl)-2-oxotetrahydropyrimidin-1(2H)-yl)methyl)-1H-pyrrolo[2,3-b]pyridin-1-yl)acetohydrazide (200 mg, 0.38 mmol) in triethyl orthoformate (2 mL) is treated with p-toluenesulfonic acid monohydrate (8 mg, 0.04 mmol). The reaction is refluxed overnight. After cooling to room temperature, the reaction is concentrated in vacuo. Purification by column chromatography over silica gel afforded 1-((1-((1,3,4-oxadiazol-2-yl)methyl)-3-chloro-1H-pyrrolo[2,3-b]pyridin-4-yl)methyl)-3-(4-methoxy-3-(pentyloxy)phenyl)tetrahydropyrimidin-2(1H)-one.

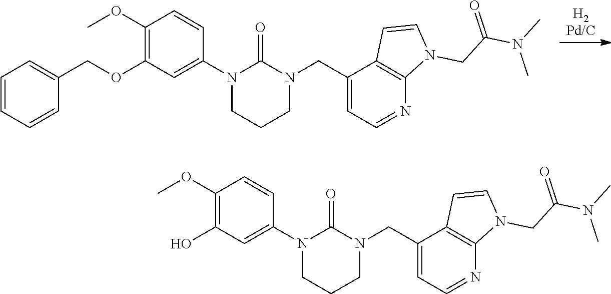

A solution of 2-(4-((3-(3-(benzyloxy)-4-methoxyphenyl)-2-oxotetrahydropyrimidin-1(2H)-yl)methyl)-1H-pyrrolo[2,3-b]pyridin-1-yl)-N,N-dimethylacetamide (150 mg, 0.28 mmol) in ethyl acetate (1.4 mL) is treated with 10 wt % palladium on carbon (30 mg). The reaction vial is purged with hydrogen, then stirred at room temperature under a hydrogen atmosphere for 3 hours. The reaction is filtered through CELITE® with ethyl acetate. The filter cake is thoroughly washed with ethyl acetate. The filtered solution is concentrated in vacuo, and purification by column chromatography over silica gel afforded 2-(4-((3-(3-hydroxy-4-methoxyphenyl)-2-oxotetrahydropyrimidin-1(2H)-yl)methyl)-1H-pyrrolo[2,3-b]pyridin-1-yl)-N,N-dimethylacetamide.

The following compounds are prepared in a similar manner as described above:

| Protected alcohol | Compound |
|---|---|
| 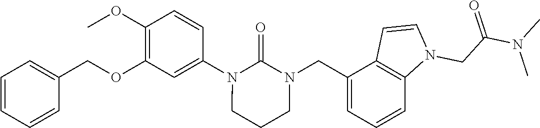 [SMILES: COc1ccc(N2CCCN(Cc3cccc4c3ccn4CC(=O)N(C)C)C2=O)cc1OCc1ccccc1] | 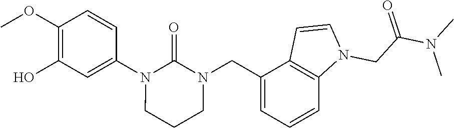 |
| 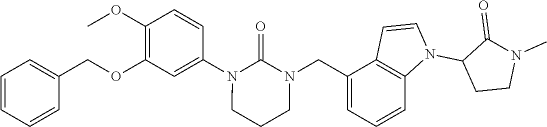 [SMILES: COc1ccc(N2CCCN(Cc3cccc4c3ccn4C3CCN(C)C3=O)C2=O)cc1OCc1ccccc1] | 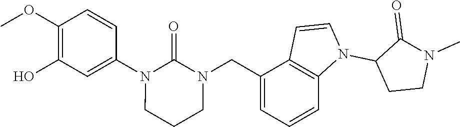 |
| 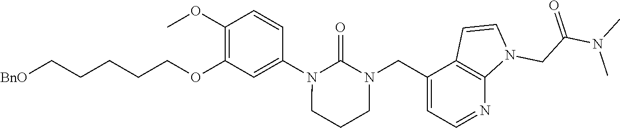 [SMILES: COc1ccc(N2CCCN(Cc3ccnc4c3ccn4CC(=O)N(C)C)C2=O)cc1OCCCCCOCc1ccccc1] | 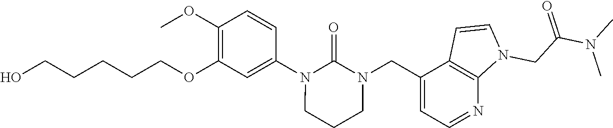 [SMILES: COc1ccc(N2CCCN(Cc3ccnc4c3ccn4CC(=O)N(C)C)C2=O)cc1OCCCCCO] |
| 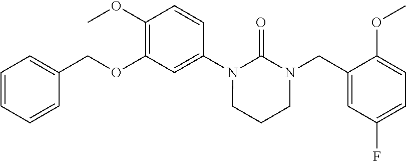 [SMILES: COc1ccc(F)cc1CN1CCCN(c2ccc(OC)c(OCc3ccccc3)c2)C1=O] | 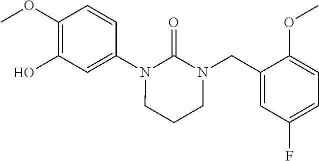 |
| 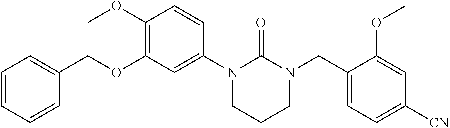 [SMILES: COc1cc(C#N)ccc1CN1CCCN(c2ccc(OC)c(OCc3ccccc3)c2)C1=O] | 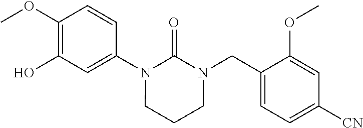 [SMILES: COc1ccc(N2CCCN(Cc3ccc(C#N)cc3OC)C2=O)cc1O] |
| 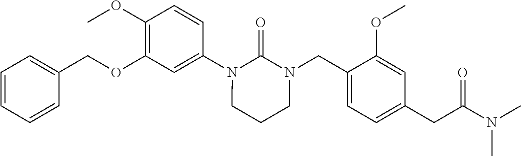 [SMILES: COc1cc(CC(=O)N(C)C)ccc1CN1CCCN(c2ccc(OC)c(OCc3ccccc3)c2)C1=O] | 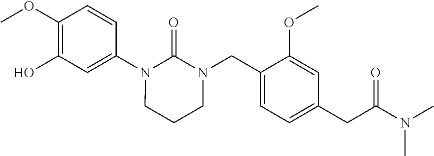 |
| 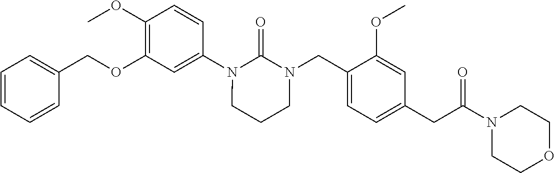 [SMILES: COc1cc(CC(=O)N2CCOCC2)ccc1CN1CCCN(c2ccc(OC)c(OCc3ccccc3)c2)C1=O] | 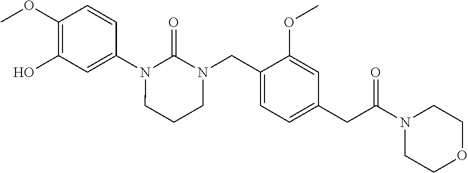 [SMILES: COc1ccc(N2CCCN(Cc3ccc(CC(=O)N4CCOCC4)cc3OC)C2=O)cc1O] |
| 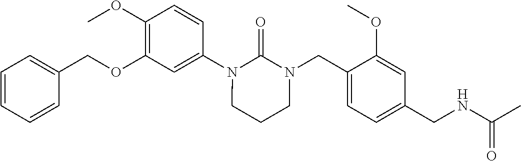 [SMILES: COc1cc(CNC(C)=O)ccc1CN1CCCN(c2ccc(OC)c(OCc3ccccc3)c2)C1=O] | 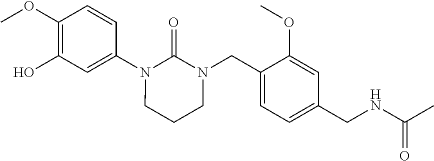 [SMILES: COc1ccc(N2CCCN(Cc3ccc(CNC(C)=O)cc3OC)C2=O)cc1O] |
| 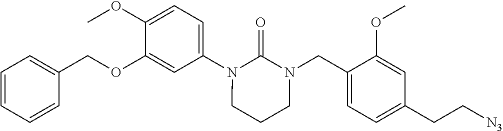 [SMILES: COc1cc(CCN=[N+]=[N-])ccc1CN1CCCN(c2ccc(OC)c(OCc3ccccc3)c2)C1=O] | 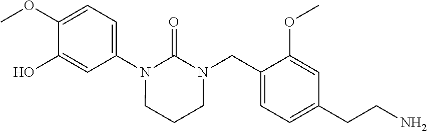 [SMILES: COc1ccc(N2CCCN(Cc3ccc(CCN)cc3OC)C2=O)cc1O] |
| 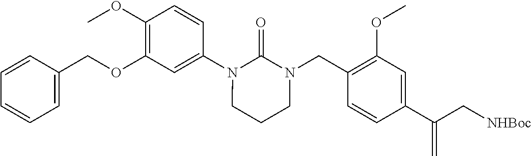 [SMILES: C=C(CNC(=O)OC(C)(C)C)c1ccc(CN2CCCN(c3ccc(OC)c(OCc4ccccc4)c3)C2=O)c(OC)c1] | 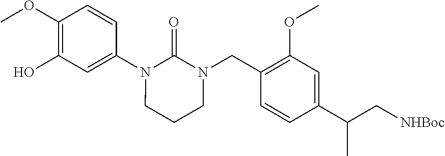 [SMILES: COc1ccc(N2CCCN(Cc3ccc(C(C)CNC(=O)OC(C)(C)C)cc3OC)C2=O)cc1O] |
| 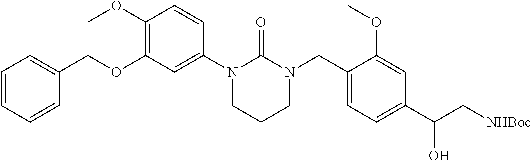 [SMILES: COc1cc(C(O)CNC(=O)OC(C)(C)C)ccc1CN1CCCN(c2ccc(OC)c(OCc3ccccc3)c2)C1=O] | 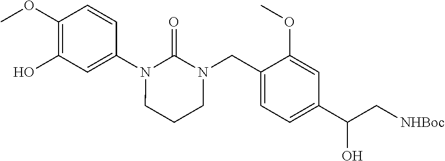 [SMILES: COc1ccc(N2CCCN(Cc3ccc(C(O)CNC(=O)OC(C)(C)C)cc3OC)C2=O)cc1O] |
| 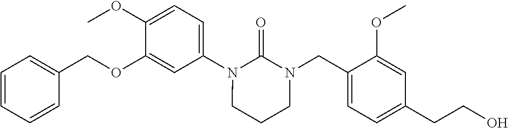 [SMILES: COc1cc(CCO)ccc1CN1CCCN(c2ccc(OC)c(OCc3ccccc3)c2)C1=O] | 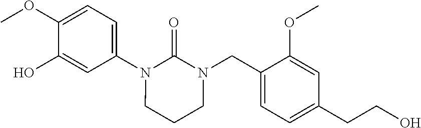 [SMILES: COc1ccc(N2CCCN(Cc3ccc(CCO)cc3OC)C2=O)cc1O] |

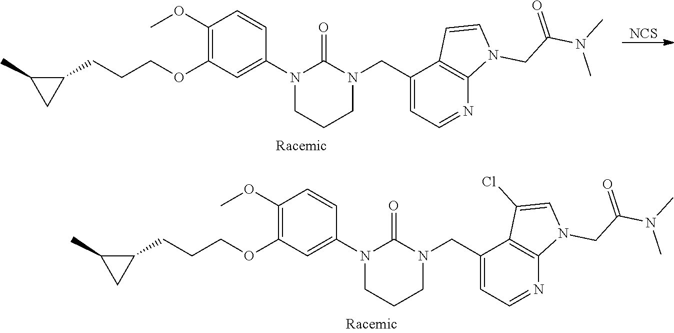

To a solution of 2-(4-((3-(4-methoxy-3-(3-((±-trans)-2-methylcyclopropyl)propoxy)phenyl)-2-oxotetrahydropyrimidin-1(2H)-yl)methyl)-1H-pyrrolo[2,3-b]pyridin-1-yl)-N,N-dimethylacetamide (0.100 g, 0.19 mmol) in dry tetrahydrofuran (4 mL) is added N-chlorosuccinimide (NCS, 27 mg, 0.20 mmol). The reaction mixture is stirred at 50° C. for 5 hours. The reaction is stopped with several drops of water. The volatiles are removed in vacuo, and purification by preparative HPLC afforded 2-(3-chloro-4-((3-(4-methoxy-3-(3-((±-trans)-2-methylcyclopropyl)propoxy)phenyl)-2-oxotetrahydropyrimidin-1(2H)-yl)methyl)-1H-pyrrolo[2,3-b]pyridin-1-yl)-N,N-dimethylacetamide.

The following compounds are prepared in a similar manner as described above:

| Heterocycle | Reagent |
|---|---|
| 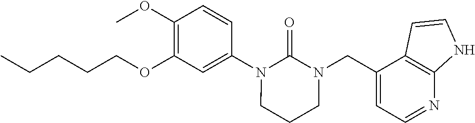 [SMILES: CCCCCOc1cc(N2CCCN(Cc3ccnc4[nH]ccc34)C2=O)ccc1OC] | 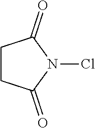 |
| 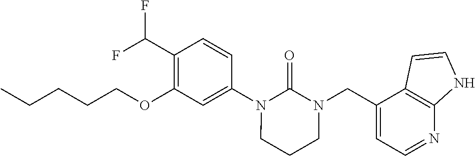 [SMILES: CCCCCOc1cc(N2CCCN(Cc3ccnc4[nH]ccc34)C2=O)ccc1C(F)F] | 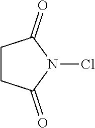 |
| 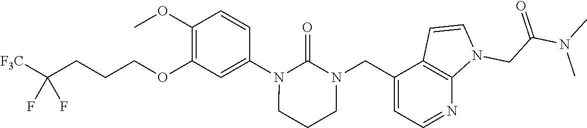 [SMILES: COc1ccc(N2CCCN(Cc3ccnc4c3ccn4CC(=O)N(C)C)C2=O)cc1OCCCC(F)(F)C(F)(F)F] | 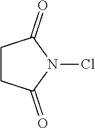 |
| 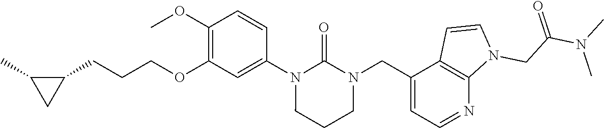 | 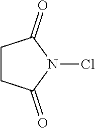 |
| 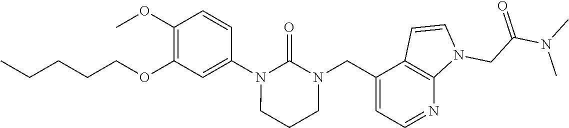 [SMILES: CCCCCOc1cc(N2CCCN(Cc3ccnc4c3ccn4CC(=O)N(C)C)C2=O)ccc1OC] | 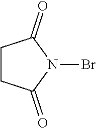 |
| 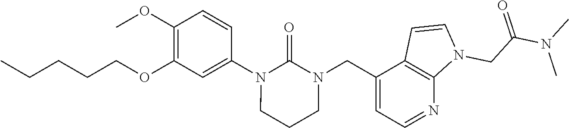 [SMILES: CCCCCOc1cc(N2CCCN(Cc3ccnc4c3ccn4CC(=O)N(C)C)C2=O)ccc1OC] | 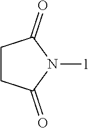 |
| 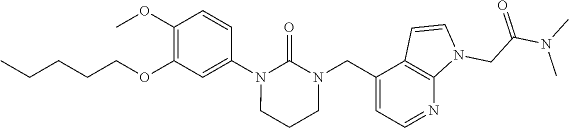 [SMILES: CCCCCOc1cc(N2CCCN(Cc3ccnc4c3ccn4CC(=O)N(C)C)C2=O)ccc1OC] | 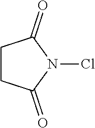 |
| 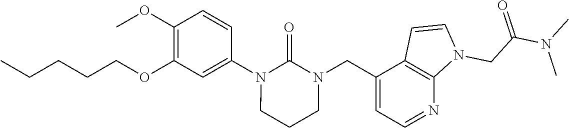 [SMILES: CCCCCOc1cc(N2CCCN(Cc3ccnc4c3ccn4CC(=O)N(C)C)C2=O)ccc1OC] | 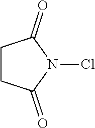 |
| 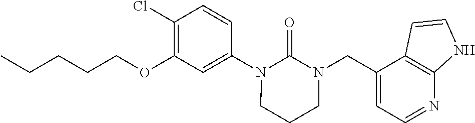 [SMILES: CCCCCOc1cc(N2CCCN(Cc3ccnc4[nH]ccc34)C2=O)ccc1Cl] | 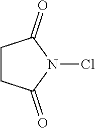 |
| 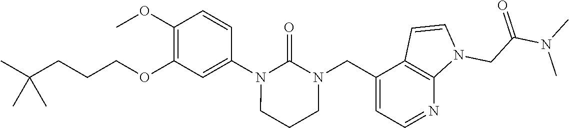 [SMILES: COc1ccc(N2CCCN(Cc3ccnc4c3ccn4CC(=O)N(C)C)C2=O)cc1OCCCC(C)(C)C] | 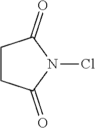 |
| 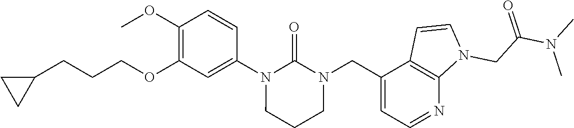 [SMILES: COc1ccc(N2CCCN(Cc3ccnc4c3ccn4CC(=O)N(C)C)C2=O)cc1OCCCC1CC1] | 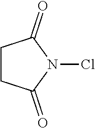 |
| 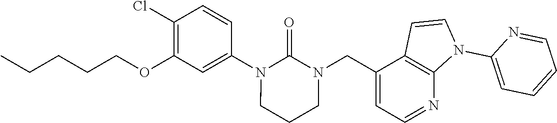 [SMILES: CCCCCOc1cc(N2CCCN(Cc3ccnc4c3ccn4-c3ccccn3)C2=O)ccc1Cl] | 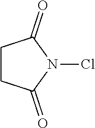 |
| 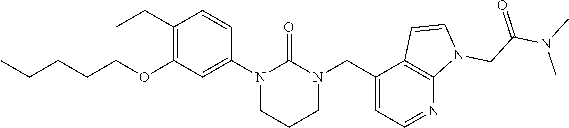 [SMILES: CCCCCOc1cc(N2CCCN(Cc3ccnc4c3ccn4CC(=O)N(C)C)C2=O)ccc1CC] | 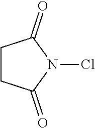 |
| 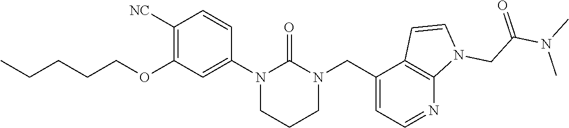 [SMILES: CCCCCOc1cc(N2CCCN(Cc3ccnc4c3ccn4CC(=O)N(C)C)C2=O)ccc1C#N] | 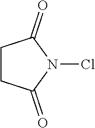 |
| 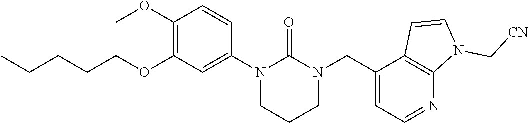 [SMILES: CCCCCOc1cc(N2CCCN(Cc3ccnc4c3ccn4CC#N)C2=O)ccc1OC] | 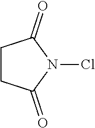 |
| 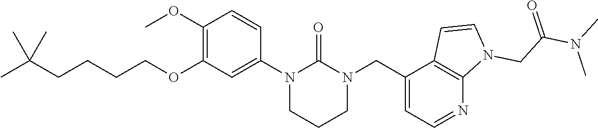 [SMILES: COc1ccc(N2CCCN(Cc3ccnc4c3ccn4CC(=O)N(C)C)C2=O)cc1OCCCCC(C)(C)C] | 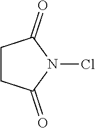 |
| 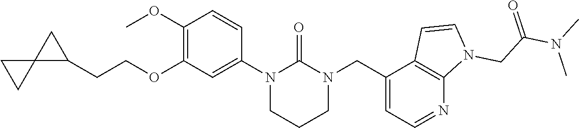 [SMILES: COc1ccc(N2CCCN(Cc3ccnc4c3ccn4CC(=O)N(C)C)C2=O)cc1OCCC1CC12CC2] | 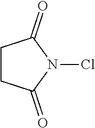 |
| 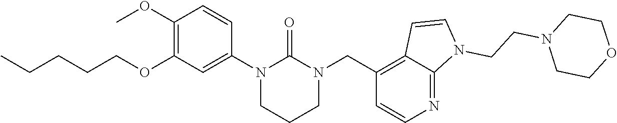 [SMILES: CCCCCOc1cc(N2CCCN(Cc3ccnc4c3ccn4CCN3CCOCC3)C2=O)ccc1OC] | 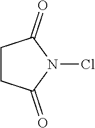 |
| 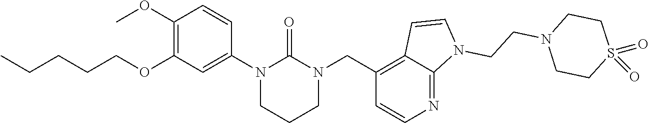 [SMILES: CCCCCOc1cc(N2CCCN(Cc3ccnc4c3ccn4CCN3CCS(=O)(=O)CC3)C2=O)ccc1OC] | 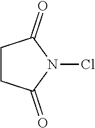 |
| 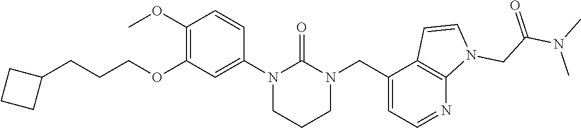 [SMILES: COc1ccc(N2CCCN(Cc3ccnc4c3ccn4CC(=O)N(C)C)C2=O)cc1OCCCC1CCC1] | 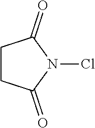 |
| 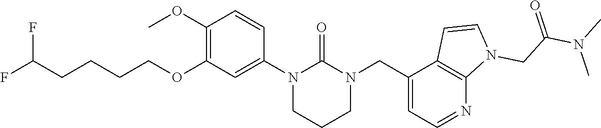 [SMILES: COc1ccc(N2CCCN(Cc3ccnc4c3ccn4CC(=O)N(C)C)C2=O)cc1OCCCCC(F)F] | 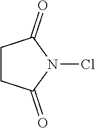 |
| 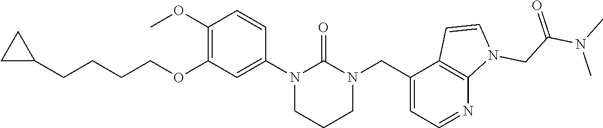 [SMILES: COc1ccc(N2CCCN(Cc3ccnc4c3ccn4CC(=O)N(C)C)C2=O)cc1OCCCCC1CC1] | 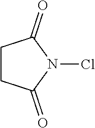 |
| 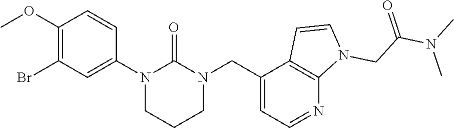 | 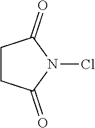 |
| 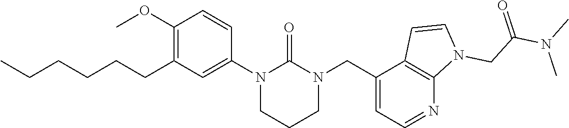 [SMILES: CCCCCCc1cc(N2CCCN(Cc3ccnc4c3ccn4CC(=O)N(C)C)C2=O)ccc1OC] | 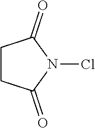 |
| 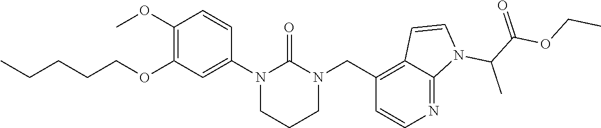 [SMILES: CCCCCOc1cc(N2CCCN(Cc3ccnc4c3ccn4C(C)C(=O)OCC)C2=O)ccc1OC] | 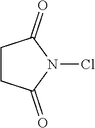 |
| 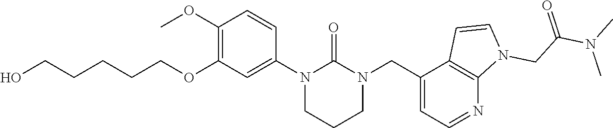 [SMILES: COc1ccc(N2CCCN(Cc3ccnc4c3ccn4CC(=O)N(C)C)C2=O)cc1OCCCCCO] | 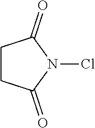 |
| 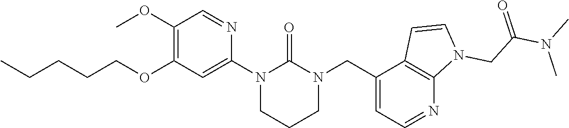 [SMILES: CCCCCOc1cc(N2CCCN(Cc3ccnc4c3ccn4CC(=O)N(C)C)C2=O)ncc1OC] | 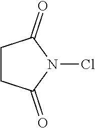 |
| 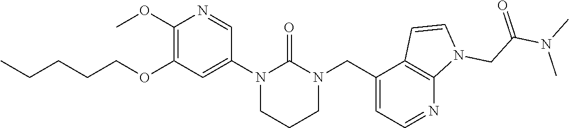 [SMILES: CCCCCOc1cc(N2CCCN(Cc3ccnc4c3ccn4CC(=O)N(C)C)C2=O)cnc1OC] | 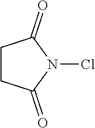 |
| 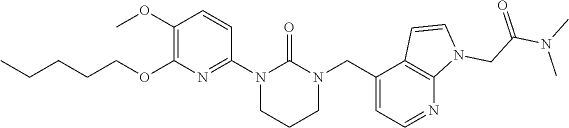 [SMILES: CCCCCOc1nc(N2CCCN(Cc3ccnc4c3ccn4CC(=O)N(C)C)C2=O)ccc1OC] | 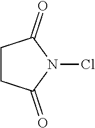 |
| 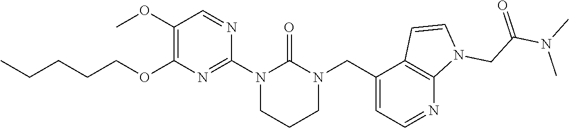 [SMILES: CCCCCOc1nc(N2CCCN(Cc3ccnc4c3ccn4CC(=O)N(C)C)C2=O)ncc1OC] | 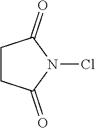 |
| 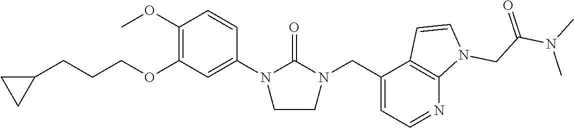 [SMILES: COc1ccc(N2CCN(Cc3ccnc4c3ccn4CC(=O)N(C)C)C2=O)cc1OCCCC1CC1] | 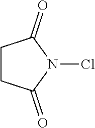 |
| 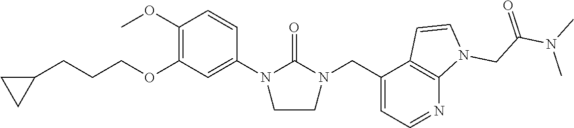 [SMILES: COc1ccc(N2CCN(Cc3ccnc4c3ccn4CC(=O)N(C)C)C2=O)cc1OCCCC1CC1] | 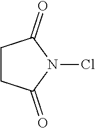 |
| 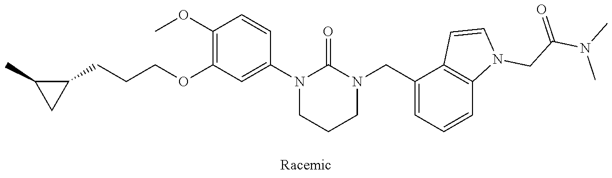 | 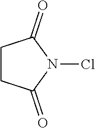 |
| 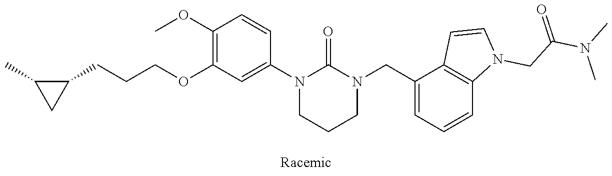 | 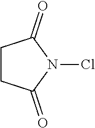 |
| 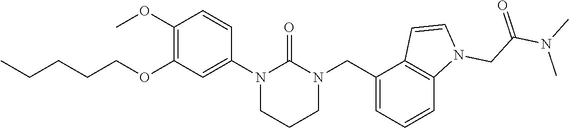 [SMILES: CCCCCOc1cc(N2CCCN(Cc3cccc4c3ccn4CC(=O)N(C)C)C2=O)ccc1OC] | 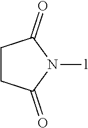 |
| 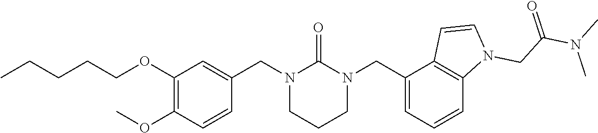 [SMILES: CCCCCOc1cc(CN2CCCN(Cc3cccc4c3ccn4CC(=O)N(C)C)C2=O)ccc1OC] | 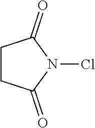 |
| 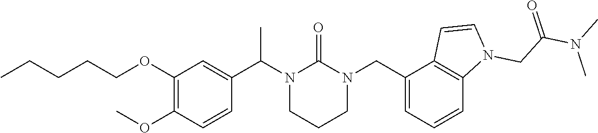 [SMILES: CCCCCOc1cc(C(C)N2CCCN(Cc3cccc4c3ccn4CC(=O)N(C)C)C2=O)ccc1OC] | 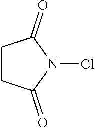 |
| 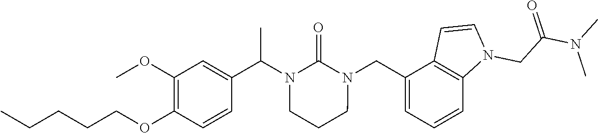 [SMILES: CCCCCOc1ccc(C(C)N2CCCN(Cc3cccc4c3ccn4CC(=O)N(C)C)C2=O)cc1OC] | 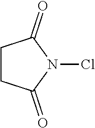 |
| 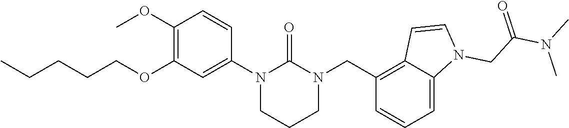 [SMILES: CCCCCOc1cc(N2CCCN(Cc3cccc4c3ccn4CC(=O)N(C)C)C2=O)ccc1OC] | 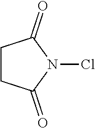 |
| 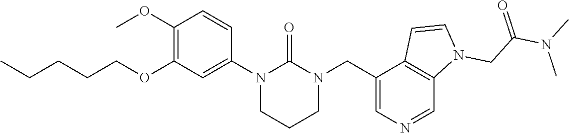 [SMILES: CCCCCOc1cc(N2CCCN(Cc3cncc4c3ccn4CC(=O)N(C)C)C2=O)ccc1OC] | 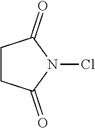 |

Compound

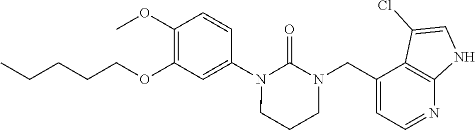 [SMILES: CCCCCOc1cc(N2CCCN(Cc3ccnc4[nH]cc(Cl)c34)C2=O)ccc1OC]

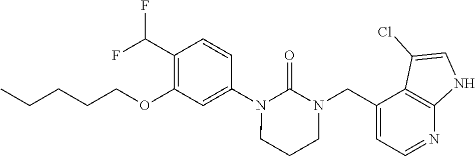 [SMILES: CCCCCOc1cc(N2CCCN(Cc3ccnc4[nH]cc(Cl)c34)C2=O)ccc1C(F)F]

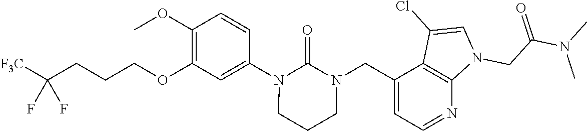 [SMILES: COc1ccc(N2CCCN(Cc3ccnc4c3c(Cl)cn4CC(=O)N(C)C)C2=O)cc1OCCCC(F)(F)C(F)(F)F]

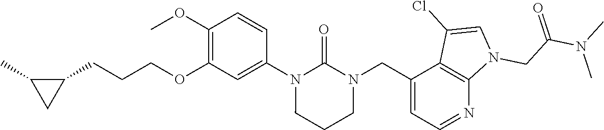

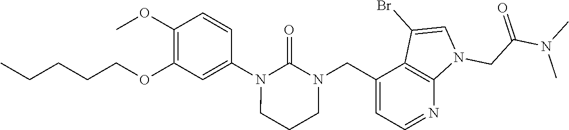 [SMILES: CCCCCOc1cc(N2CCCN(Cc3ccnc4c3c(Br)cn4CC(=O)N(C)C)C2=O)ccc1OC]

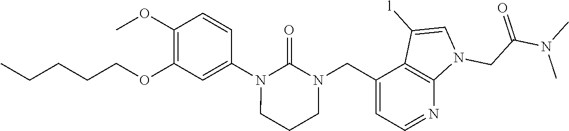

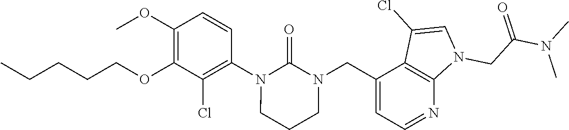 [SMILES: CCCCCOc1c(OC)ccc(N2CCCN(Cc3ccnc4c3c(Cl)cn4CC(=O)N(C)C)C2=O)c1Cl]

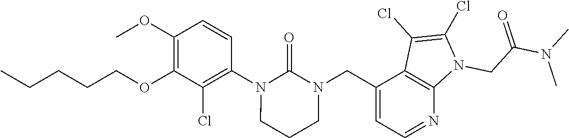 [SMILES: CCCCCOc1c(OC)ccc(N2CCCN(Cc3ccnc4c3c(Cl)c(Cl)n4CC(=O)N(C)C)C2=O)c1Cl]

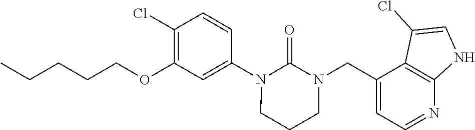 [SMILES: CCCCCOc1cc(N2CCCN(Cc3ccnc4[nH]cc(Cl)c34)C2=O)ccc1Cl]

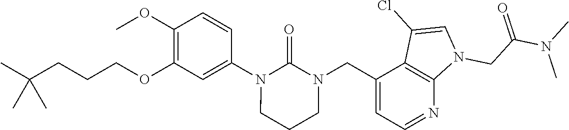 [SMILES: COc1ccc(N2CCCN(Cc3ccnc4c3c(Cl)cn4CC(=O)N(C)C)C2=O)cc1OCCCC(C)(C)C]

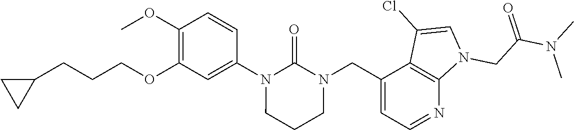 [SMILES: COc1ccc(N2CCCN(Cc3ccnc4c3c(Cl)cn4CC(=O)N(C)C)C2=O)cc1OCCCC1CC1]

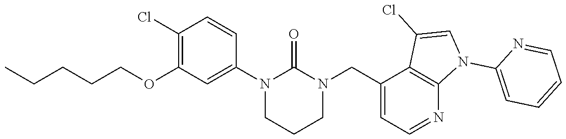 [SMILES: CCCCCOc1cc(N2CCCN(Cc3ccnc4c3c(Cl)cn4-c3ccccn3)C2=O)ccc1Cl]

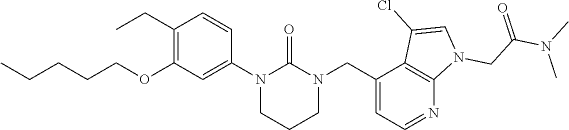 [SMILES: CCCCCOc1cc(N2CCCN(Cc3ccnc4c3c(Cl)cn4CC(=O)N(C)C)C2=O)ccc1CC]

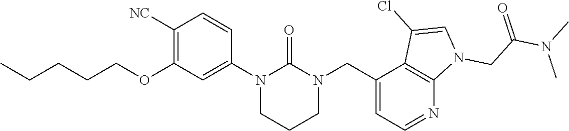 [SMILES: CCCCCOc1cc(N2CCCN(Cc3ccnc4c3c(Cl)cn4CC(=O)N(C)C)C2=O)ccc1C#N]

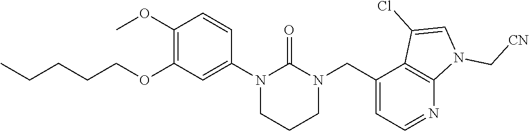 [SMILES: CCCCCOc1cc(N2CCCN(Cc3ccnc4c3c(Cl)cn4CC#N)C2=O)ccc1OC]

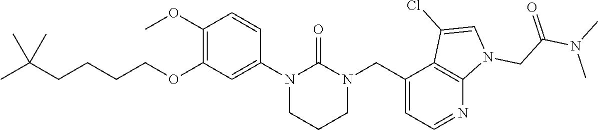 [SMILES: COc1ccc(N2CCCN(Cc3ccnc4c3c(Cl)cn4CC(=O)N(C)C)C2=O)cc1OCCCCC(C)(C)C]

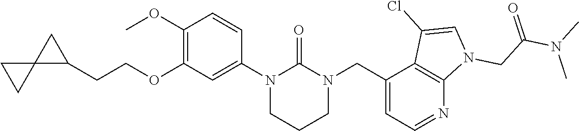 [SMILES: COc1ccc(N2CCCN(Cc3ccnc4c3c(Cl)cn4CC(=O)N(C)C)C2=O)cc1OCCC1CC12CC2]

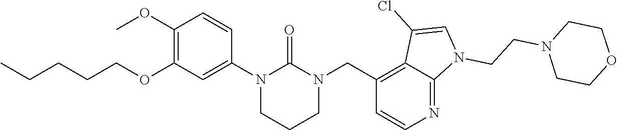 [SMILES: CCCCCOc1cc(N2CCCN(Cc3ccnc4c3c(Cl)cn4CCN3CCOCC3)C2=O)ccc1OC]

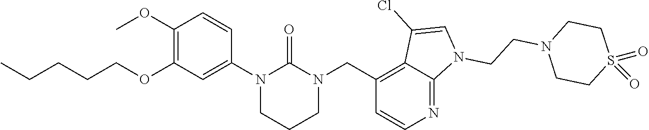 [SMILES: CCCCCOc1cc(N2CCCN(Cc3ccnc4c3c(Cl)cn4CCN3CCS(=O)(=O)CC3)C2=O)ccc1OC]

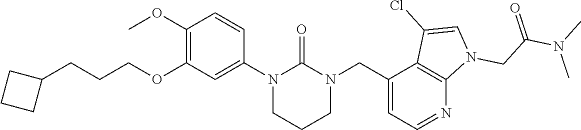 [SMILES: COc1ccc(N2CCCN(Cc3ccnc4c3c(Cl)cn4CC(=O)N(C)C)C2=O)cc1OCCCC1CCC1]

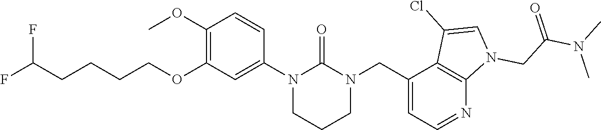 [SMILES: COc1ccc(N2CCCN(Cc3ccnc4c3c(Cl)cn4CC(=O)N(C)C)C2=O)cc1OCCCCC(F)F]

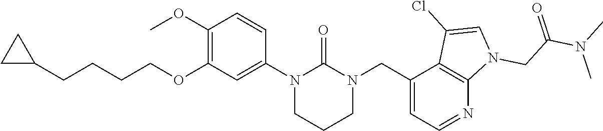 [SMILES: COc1ccc(N2CCCN(Cc3ccnc4c3c(Cl)cn4CC(=O)N(C)C)C2=O)cc1OCCCCC1CC1]

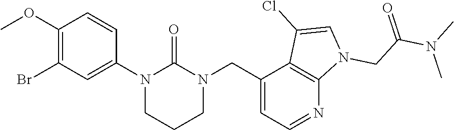

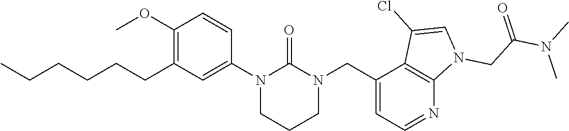 [SMILES: CCCCCCc1cc(N2CCCN(Cc3ccnc4c3c(Cl)cn4CC(=O)N(C)C)C2=O)ccc1OC]

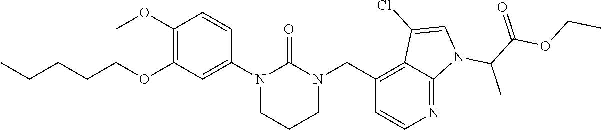 [SMILES: CCCCCOc1cc(N2CCCN(Cc3ccnc4c3c(Cl)cn4C(C)C(=O)OCC)C2=O)ccc1OC]

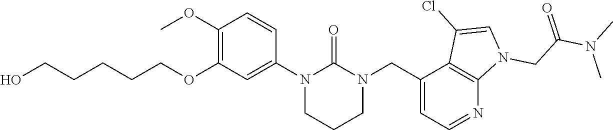 [SMILES: COc1ccc(N2CCCN(Cc3ccnc4c3c(Cl)cn4CC(=O)N(C)C)C2=O)cc1OCCCCCO]

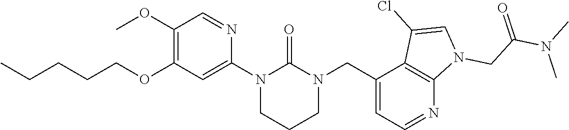 [SMILES: CCCCCOc1cc(N2CCCN(Cc3ccnc4c3c(Cl)cn4CC(=O)N(C)C)C2=O)ncc1OC]

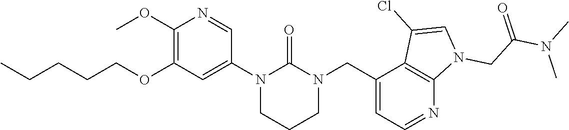 [SMILES: CCCCCOc1cc(N2CCCN(Cc3ccnc4c3c(Cl)cn4CC(=O)N(C)C)C2=O)cnc1OC]

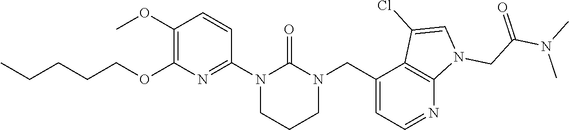 [SMILES: CCCCCOc1nc(N2CCCN(Cc3ccnc4c3c(Cl)cn4CC(=O)N(C)C)C2=O)ccc1OC]

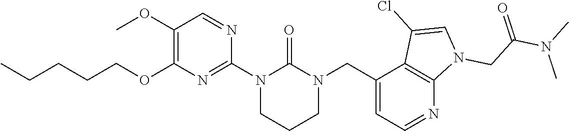 [SMILES: CCCCCOc1nc(N2CCCN(Cc3ccnc4c3c(Cl)cn4CC(=O)N(C)C)C2=O)ncc1OC]

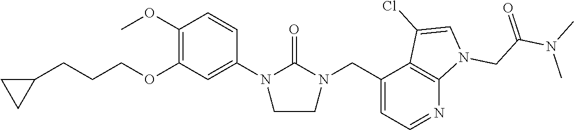 [SMILES: COc1ccc(N2CCN(Cc3ccnc4c3c(Cl)cn4CC(=O)N(C)C)C2=O)cc1OCCCC1CC1]

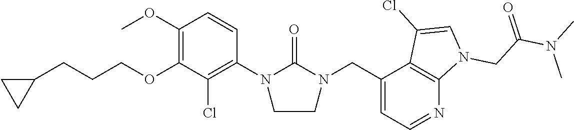 [SMILES: COc1ccc(N2CCN(Cc3ccnc4c3c(Cl)cn4CC(=O)N(C)C)C2=O)c(Cl)c1OCCCC1CC1]

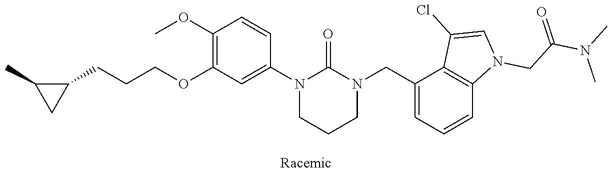

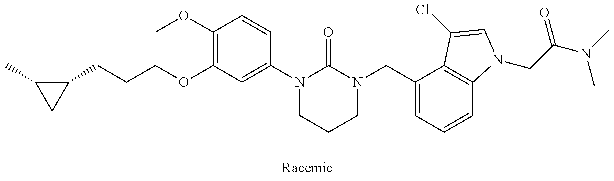

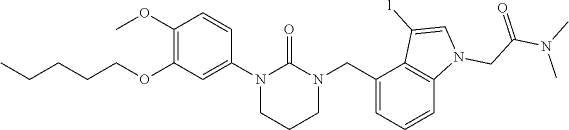

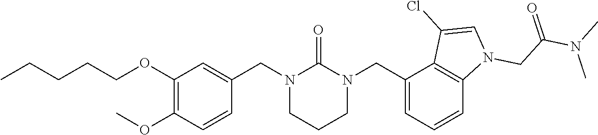 [SMILES: CCCCCOc1cc(CN2CCCN(Cc3cccc4c3c(Cl)cn4CC(=O)N(C)C)C2=O)ccc1OC]

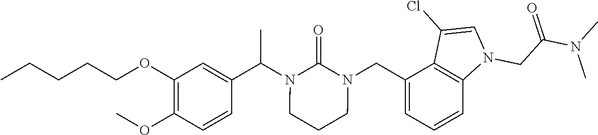 [SMILES: CCCCCOc1cc(C(C)N2CCCN(Cc3cccc4c3c(Cl)cn4CC(=O)N(C)C)C2=O)ccc1OC]

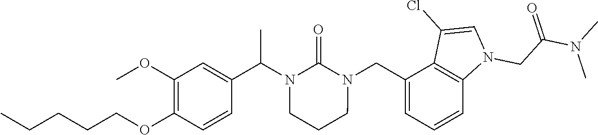 [SMILES: CCCCCOc1ccc(C(C)N2CCCN(Cc3cccc4c3c(Cl)cn4CC(=O)N(C)C)C2=O)cc1OC]

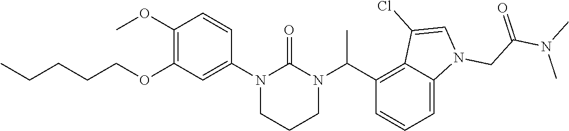 [SMILES: CCCCCOc1cc(N2CCCN(C(C)c3cccc4c3c(Cl)cn4CC(=O)N(C)C)C2=O)ccc1OC]

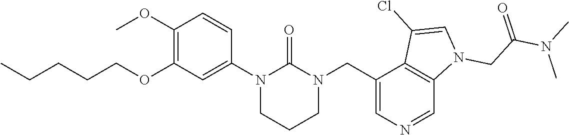 [SMILES: CCCCCOc1cc(N2CCCN(Cc3cncc4c3c(Cl)cn4CC(=O)N(C)C)C2=O)ccc1OC]

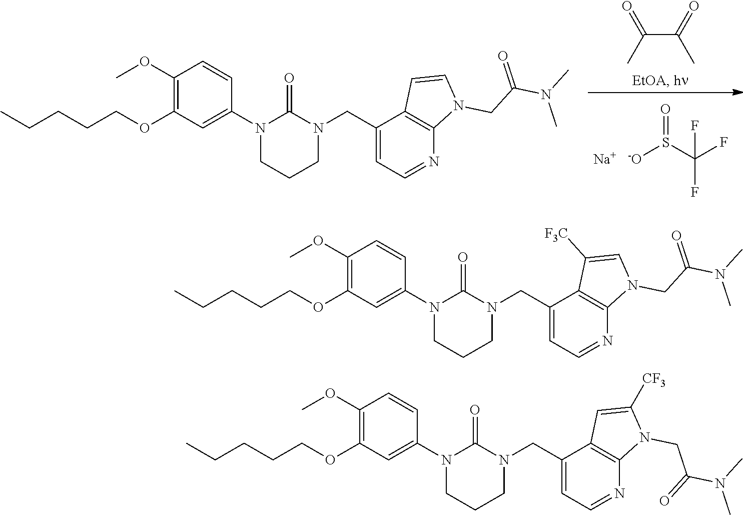

A solution of 2-(4-((3-(4-methoxy-3-(pentyloxy)phenyl)-oxotetrahydropyrimidin-1(2H)-yl)methyl)-1H-pyrrolo[2,3-b]pyridin-1-yl)-N,N-dimethylacetamide (50.0 mg, 0.099 mmol), sodium trifluoromethanesulfinate (19.0 mg, 0.12 mmol) and 2,3-butanedione (200 uL, 2.2 mmol) in ethyl acetate (0.8 mL) is irradiated overnight using a household LED light bulb. The reaction is concentrated in vacuo. Purification by preparative HPLC afforded 2-(4-((3-(4-methoxy-3-(pentyloxy)phenyl)-2-oxotetrahydropyrimidin-1(2H)-yl)methyl)-3-(trifluoromethyl)-1H-pyrrolo[2,3-b]pyridin-1-yl)-N,N-dimethylacetamide & 2-(4-((3-(4-methoxy-3-(pentyloxy)phenyl)-2-oxotetrahydropyrimidin-1(2H)-yl)methyl)-2-(trifluoromethyl)-1H-pyrrolo[2,3-b]pyridin-1-yl)-N,N-dimethylacetamide.

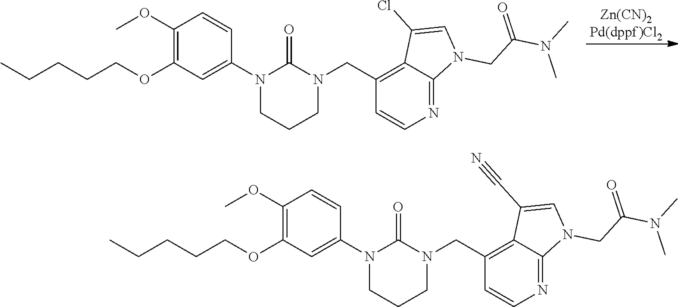

To a solution of 2-(3-bromo-4-((3-(4-methoxy-3-(pentyloxy)phenyl)-2-oxotetrahydropyrimidin-1(2H)-yl)methyl)-1H-pyrrolo[2,3-b]pyridin-1-yl)-N,N-dimethylacetamide (35 mg, 0.06 mol) in anhydrous N,N-dimethylformamide (3 mL) is added zinc(II) cyanide (7 mg, 0.06 mol), zinc dust (11.7 mg, 0.18 mmol) and 1,1′-bis(diphenylphosphino)ferrocene]dichloropalladium(II) dichloromethane complex (5 mg, 0.01 mmol) at room temperature. The mixture is heated to 150° C. and stirred for 12 hours at this temperature. The reaction is cooled to room temperature and quenched with a minimal amount of water. The suspension is filtered through CELITE® with a minimal amount of N,N-dimethylformamide. The filtered solution is purified directly by preparative HPLC to afford 2-(3-cyano-4-((3-(4-methoxy-3-(pentyloxy)phenyl)-2-oxotetrahydropyrimidin-1(2H)-yl)methyl)-1H-pyrrolo[2,3-b]pyridin-1-yl)-N,N-dimethylacetamide.

The following compounds are prepared in a similar manner as described above.

| Aryl halide | Compound |
|---|---|
| 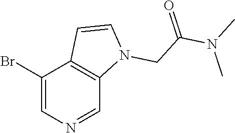 [SMILES: CN(C)C(=O)Cn1ccc2c(Br)cncc21] | 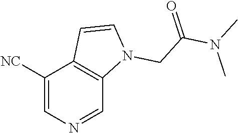 [SMILES: CN(C)C(=O)Cn1ccc2c(C#N)cncc21] |
| 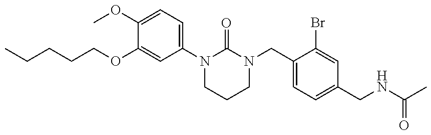 [SMILES: CCCCCOc1cc(N2CCCN(Cc3ccc(CNC(C)=O)cc3Br)C2=O)ccc1OC] | 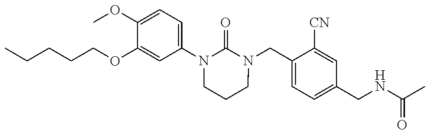 [SMILES: CCCCCOc1cc(N2CCCN(Cc3ccc(CNC(C)=O)cc3C#N)C2=O)ccc1OC] |

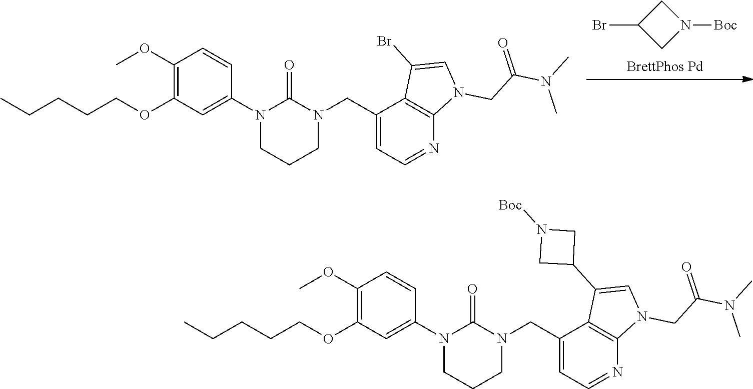

Following a modified protocol reported by Bhonde, et al. (Angew. Chem. Int. Ed. 2016, 55, 1849): A round bottom flask is charged with 2-(3-bromo-4-((3-(4-methoxy-3-(pentyloxy)phenyl)-2-oxotetrahydropyrimidin-1(2H)-yl)methyl)-1H-pyrrolo[2,3-b]pyridin-1-yl)-N,N-dimethylacetamide (200 mg, 0.34 mmol), sodium chloride (60 mg, 0.68 mmol), sodium octanoate (56 mg, 0.34 mmol), zinc powder (55 mg, 0.85 mmol) and crotyl(2-dicyclohexylphosphino-2′,4′,6′-triisopropyl-3,6-dimethoxy-1,1′-biphenyl)palladium(II) triflate (BrettPhos Pd(crotyl)]OTf, 14 mg, 0.02 mmol). The flask is evacuated and purged with argon three times. tert-butyl 3-bromoazetidine-1-carboxylate (160 mg, 0.68 mmol), N,N,N,N′-tetramethylethylenediamine (TMEDA, 127 uL, 0.85 mmol), methyl octanoate (61 uL, 0.34 mmol), and water (1.1 mL) are then added. The reaction is heated to 100° C. and stirred overnight at this temperature. The reaction is cooled to room temperature then diluted with ethyl acetate. The solids are removed by filtration over CELITE®. The filter cake is thoroughly washed with additional ethyl acetate. The filtered solution is washed with 0.3N hydrochloric acid then 0.3N aqueous sodium hydroxide. The organic phase is dried over anhydrous sodium sulfate, filtered, and concentrated in vacuo. Purification by preparative HPLC afforded tert-butyl 3-(1-(2-(dimethylamino)-2-oxoethyl)-4-((3-(4-methoxy-3-(pentyloxy)phenyl)-2-oxotetrahydropyrimidin-1(2H)-yl)methyl)-1H-pyrrolo[2,3-b]pyridin-3-yl)azetidine-1-carboxylate.

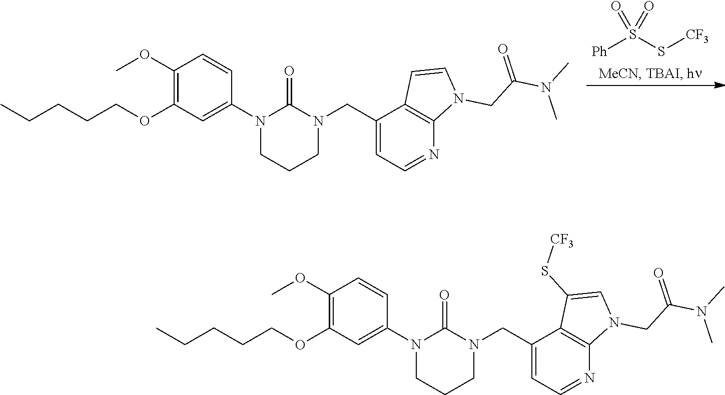

Following a modified protocol reported by Li. et al. (Chem. Sci., 2018, 9, 5781): A 20-mL vial is charged with 2-(4-((3-(4-methoxy-3-(pentyloxy)phenyl)-2-oxotetrahydropyrimidin-1(2H)-yl)methyl)-1H-pyrrolo[2,3-b]pyridin-1-yl)-N,N-dimethylacetamide (150 mg, 0.30 mmol) and S-(trifluoromethyl) benzenesulfonothioate (143 mg, 0.59 mmol). The vial is evacuated and purged with argon three times. A solution of tetrabutylammonium iodide (22 mg, 0.06 mmol) in degassed, anhydrous acetonitrile (3 mL) is added to the vial. The reaction is stirred at room temperature overnight while being irradiated by a household compact fluorescent lightbulb (CFL). The reaction is diluted with ethyl acetate, then filtered through CELITE®. The filter cake is thoroughly washed with ethyl acetate. The filtered solution is concentrated in vacuo. Purification by column chromatography over reversed-phase C18 silica gel afforded 2-(4-((3-(4-methoxy-3-(pentyloxy)phenyl)-2-oxotetrahydropyrimidin-1(2H)-yl)methyl)-3-((trifluoromethyl)thio)-1H-pyrrolo[2,3-b]pyridin-1-yl)-N,N-dimethylacetamide.

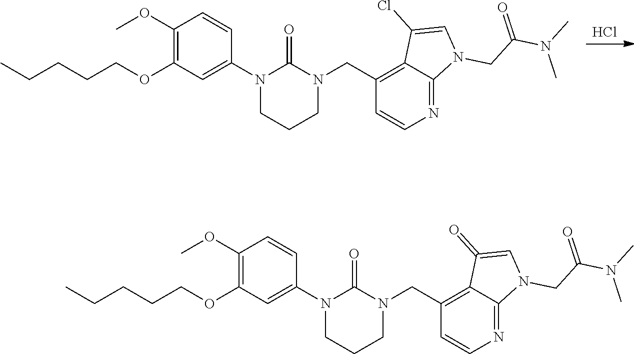

A solution of 2-(3-chloro-4-((3-(4-methoxy-3-(pentyloxy)phenyl)-2-oxotetrahydropyrimidin-1(2H)-yl)methyl)-1H-pyrrolo[2,3-b]pyridin-1-yl)-N,N-dimethylacetamide (150 mg, 0.28 mmol) in 1,4-dioxane (2.8 mL) is treated with 1M hydrochloric acid. The reaction is refluxed for 1 hour then concentrated in vacuo. Purification by preparative HPLC afforded 2-(4-((3-(4-methoxy-3-(pentyloxy)phenyl)-2-oxotetrahydropyrimidin-1(2H)-yl)methyl)-3-oxo-2,3-dihydro-1H-pyrrolo[2,3-b]pyridin-1-yl)-N,N-dimethylacetamide.

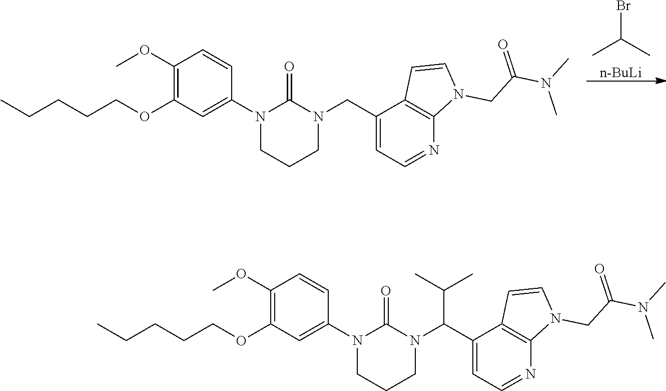

A solution of 2-(4-((3-(4-methoxy-3-(pentyloxy)phenyl)-2-oxotetrahydropyrimidin-1(2H)-yl)methyl)-1H-pyrrolo[2,3-b]pyridin-1-yl)-N,N-dimethylacetamide (175 mg, 0.34 mmol) in anhydrous tetrahydrofuran (1.7 mL) is cooled to −78° C. n-Butyllithium (1.6M in hexanes, 0.26 mL, 0.41 mmol) is added dropwise at −78° C. The reaction is stirred at −78° C. for 15 minutes before adding neat 2-bromopropane (35 uL, 0.37 mmol). The reaction is stirred at −78° C. for 4 hours, then quenched at this temperature with saturated aqueous ammonium chloride solution. After warming to room temperature, the reaction is extracted with dichloromethane. The combined organic layers are dried over anhydrous sodium sulfate, filtered, and concentrated in vacuo. Purification by preparative HPLC afforded 2-(4-(1-(3-(4-methoxy-3-(pentyloxy)phenyl)-2-oxotetrahydropyrimidin-1(2H)-yl)-2-methylpropyl)-1H-pyrrolo[2,3-b]pyridin-1-yl)-N,N-dimethylacetamide.

The following compounds are prepared in a similar manner as described above:

| Heterocycle | Electrophile |
|---|---|
| 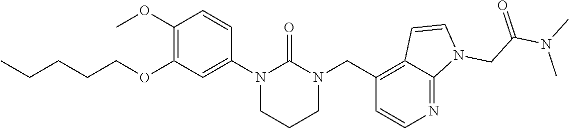 [SMILES: CCCCCOc1cc(N2CCCN(Cc3ccnc4c3ccn4CC(=O)N(C)C)C2=O)ccc1OC] | 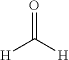 |
| 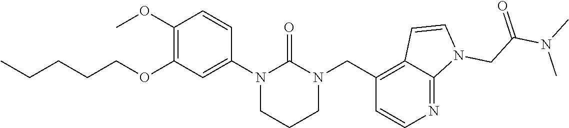 [SMILES: CCCCCOc1cc(N2CCCN(Cc3ccnc4c3ccn4CC(=O)N(C)C)C2=O)ccc1OC] | MeI |

Compound

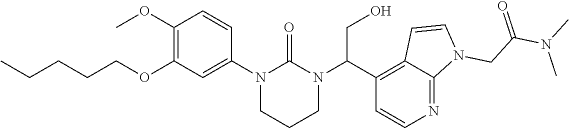 [SMILES: CCCCCOc1cc(N2CCCN(C(CO)c3ccnc4c3ccn4CC(=O)N(C)C)C2=O)ccc1OC]

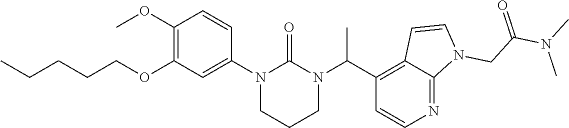 [SMILES: CCCCCOc1cc(N2CCCN(C(C)c3ccnc4c3ccn4CC(=O)N(C)C)C2=O)ccc1OC]

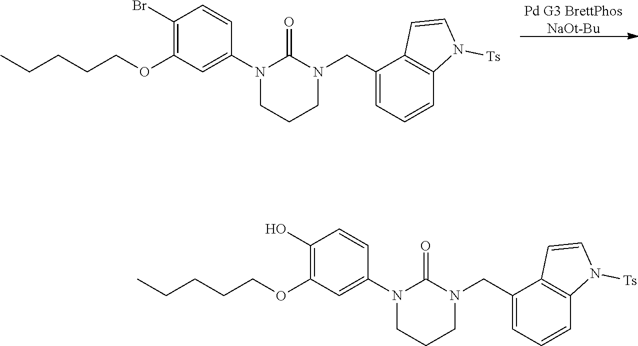 [SMILES: CCCCCOc1cc(N2CCCN(Cc3cccc4c3ccn4S(=O)(=O)c3ccc(C)cc3)C2=O)ccc1Br ; "Pd G3 BrettPhosNaOt-Bu" ; CCCCCOc1cc(N2CCCN(Cc3cccc4c3ccn4S(=O)(=O)c3ccc(C)cc3)C2=O)ccc1O]

A 2-mL vial is charged with 1-(4-bromo-3-(pentyloxy)phenyl)-3-((1-tosyl-1H-indol-4-yl)methyl)tetrahydropyrimidin-2(1H)-one (310 mg, 0.50 mmol), sodium tert-butoxide (98 mg, 0.99 mmol) and [(2-di-tert-butylphosphino-3,6-dimethoxy-2′,4′,6′-triisopropyl-1,1′-biphenyl)-2-(2′-amino-1,1′-biphenyl)]palladium(II) methanesulfonate (tBuBrettPhos Pd G3, 9 mg, 0.01 mmol). The vial is purged with nitrogen, then 1,4-dioxane (496 uL) and then water (45 uL, 2.5 mmol) are added by syringe. The reaction is heated to 90° C. and stirred at this temperature overnight. After cooling to room temperature, the reaction is filtered through CELITE® with ethyl acetate. The filter cake is thoroughly washed with ethyl acetate. The filtered solution is concentrated in vacuo. Purification by column chromatography over reversed-phase C18 silica gel afforded 1-(4-hydroxy-3-(pentyloxy)phenyl)-3-((1-tosyl-1H-indol-4-yl)methyl)tetrahydropyrimidin-2(1H)-one.

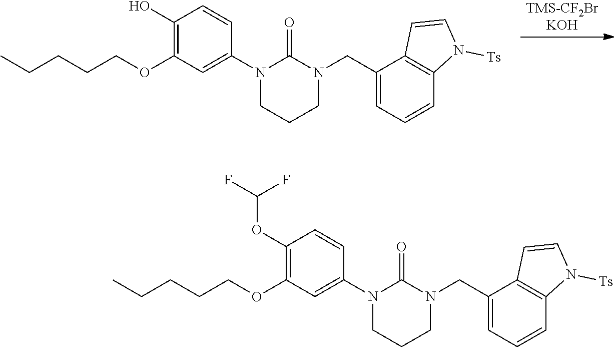 [SMILES: "TMS-CF2BrKOH" ; CCCCCOc1cc(N2CCCN(Cc3cccc4c3ccn4S(=O)(=O)c3ccc(C)cc3)C2=O)ccc1O ; CCCCCOc1cc(N2CCCN(Cc3cccc4c3ccn4S(=O)(=O)c3ccc(C)cc3)C2=O)ccc1OC(F)F]

To a solution of 1-(4-hydroxy-3-(pentyloxy)phenyl)-3-((1-tosyl-1H-indol-4-yl)methyl)tetrahydropyrimidin-2(1H)-one (120 mg, 0.21 mmol) in anhydrous dichloromethane (0.86 mL) at 0° C. is added 20% aqueous potassium hydroxide solution (0.36 mL, 1.28 mmol) followed by (bromodifluoromethyl)trimethylsilane (68 μL, 0.43 mmol). The reaction is stirred overnight while gradually warming to room temperature. The reaction is quenched with saturated aqueous ammonium chloride solution, then extracted with ethyl acetate. The combined organic layers are washed with brine, then dried over anhydrous sodium sulfate, filtered, and concentrated in vacuo. Purification by column chromatography over reversed-phase C18 silica gel afforded 1-(4-(difluoromethoxy)-3-(pentyloxy)phenyl)-3-((1-tosyl-1H-indol-4-yl)methyl)tetrahydropyrimidin-2(1H)-one.

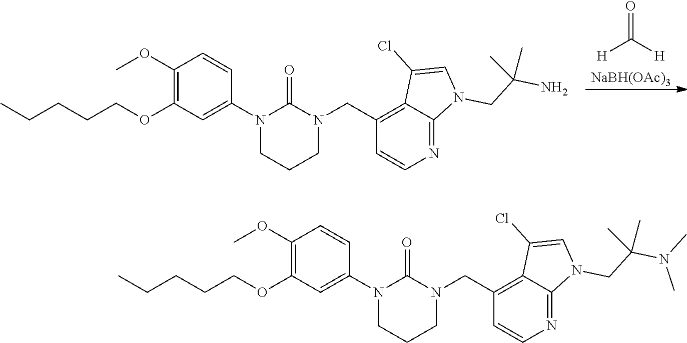 [SMILES: CCCCCOc1cc(N2CCCN(Cc3ccnc4c3c(Cl)cn4CC(C)(C)N)C2=O)ccc1OC ; C=O ; CCCCCOc1cc(N2CCCN(Cc3ccnc4c3c(Cl)cn4CC(C)(C)N(C)C)C2=O)ccc1OC ; "NaBH(OAc)3"]

A solution of 1-((1-(2-amino-2-methylpropyl)-3-chloro-1H-pyrrolo[2,3-b]pyridin-4-yl)methyl)-3-(4-methoxy-3-(pentyloxy)phenyl)tetrahydropyrimidin-2(1H)-one (55 mg, 0.10 mmol) in methanol (0.5 mL) is treated with formaldehyde (37 wt % in water, 22 uL, 0.35 mmol), sodium triacetoxyborohydride (53 mg, 0.25 mmol) and glacial acetic acid (1 uL). The reaction is stirred at room temperature overnight. More formaldehyde (22 uL, 0.35 mmol) and sodium triacetoxyborohydride (53 mg, 0.25 mmol) are added. The reaction is stirred another 2 hours at room temperature. The volatiles are removed in vacuo. The residue is diluted with saturated aqueous sodium bicarbonate then extracted with dichloromethane. The combined organic layers are dried over anhydrous sodium sulfate, filtered, and concentrated in vacuo. Purification by column chromatography over silica gel afforded 1-((3-chloro-1-(2-(dimethylamino)-2-methylpropyl)-1H-pyrrolo[2,3-b]pyridin-4-yl)methyl)-3-(4-methoxy-3-(pentyloxy)phenyl)tetrahydropyrimidin-2(1H)-one.

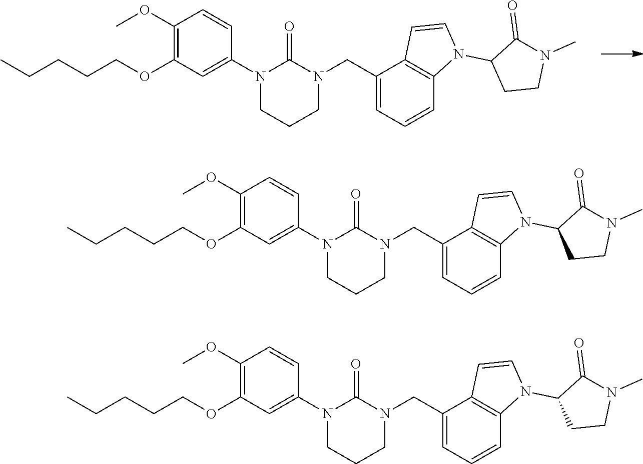

Racemic 1-(4-methoxy-3-(pentyloxy)phenyl)-3-((1-(1-methyl-2-oxopyrrolidin-3-yl)-1H-indol-4-yl)methyl)tetrahydropyrimidin-2(1H)-one (210 mg, 0.40 mmol) is purified by preparative chiral SFC to afford pure enantiomers. (Column: Lux Amylose-2, 10×250 mm, 5 um, 60% methanol, 10 mL/min, 150 bar; Column Temp: 40° C.; run time: 25 min). t_{R} enantiomer 1=13.6 min. t_{R} enantiomer 2=20.5 min.

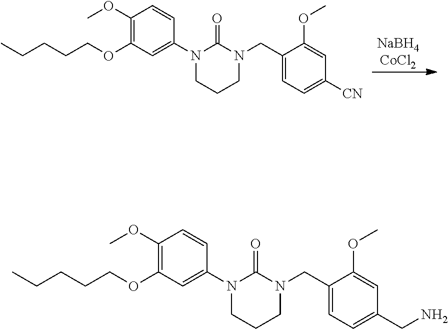 [SMILES: CCCCCOc1cc(N2CCCN(Cc3ccc(C#N)cc3OC)C2=O)ccc1OC ; "NaBH4CoCl2" ; CCCCCOc1cc(N2CCCN(Cc3ccc(CN)cc3OC)C2=O)ccc1OC]

To a solution of 3-methoxy-4-((3-(4-methoxy-3-(pentyloxy)phenyl)-2-oxotetrahydropyrimidin-1(2H)-yl)methyl)benzonitrile (5.3 g, 12 mmol) in methanol (100 mL) is cooled to at −30° C. before adding cobalt chloride hexahydrate (23.7 g, 100 mmol) at −30′° C. The mixture is stirred for 30 minutes at this temperature, then sodium borohydride (1.5 g, 200 mmol) is added in portions while maintaining the temperature between −30 and −20° C. After complete addition, the reaction is stirred for another hour at −30° C., then warmed to room temperature and stirred for 2 additional hours. The mixture is cooled to 0° C. and quenched with water. The resulting mixture is extracted with ethyl acetate. The combined organic phases are washed with brine, dried over anhydrous sodium sulfate, filtered, and concentrated in vacuo. Purification by column chromatography over silica gel afforded 1-(4-(aminomethyl)-2-methoxybenzyl)-3-(4-methoxy-3-(pentyloxy)phenyl)tetrahydropyrimidin-2(1H)-one.

The following compounds are prepared in a similar manner as described above.

| Nitrile | Compound |
|---|---|
| 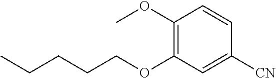 [SMILES: CCCCCOc1cc(C#N)ccc1OC] | 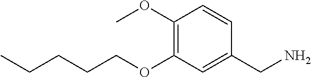 [SMILES: CCCCCOc1cc(CN)ccc1OC] |
| 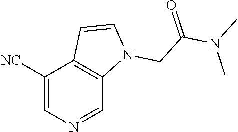 [SMILES: CN(C)C(=O)Cn1ccc2c(C#N)cncc21] | 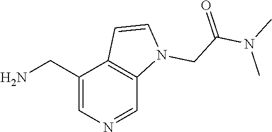 [SMILES: CN(C)C(=O)Cn1ccc2c(CN)cncc21] |
| 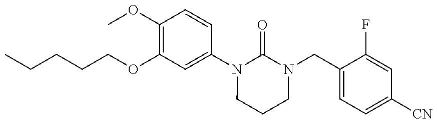 [SMILES: CCCCCOc1cc(N2CCCN(Cc3ccc(C#N)cc3F)C2=O)ccc1OC] | 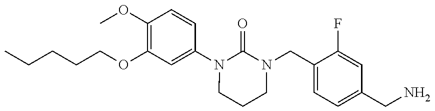 [SMILES: CCCCCOc1cc(N2CCCN(Cc3ccc(CN)cc3F)C2=O)ccc1OC] |
| 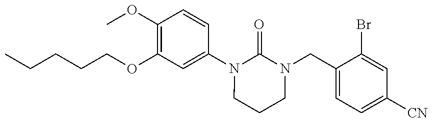 [SMILES: CCCCCOc1cc(N2CCCN(Cc3ccc(C#N)cc3Br)C2=O)ccc1OC] | 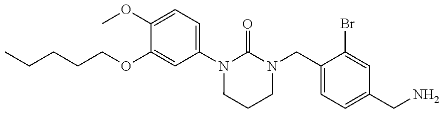 [SMILES: CCCCCOc1cc(N2CCCN(Cc3ccc(CN)cc3Br)C2=O)ccc1OC] |
| 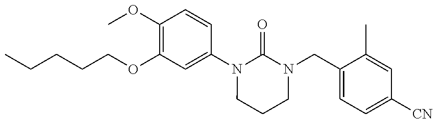 [SMILES: CCCCCOc1cc(N2CCCN(Cc3ccc(C#N)cc3C)C2=O)ccc1OC] | 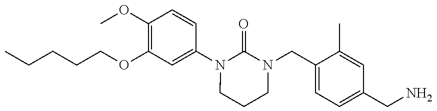 [SMILES: CCCCCOc1cc(N2CCCN(Cc3ccc(CN)cc3C)C2=O)ccc1OC] |
| 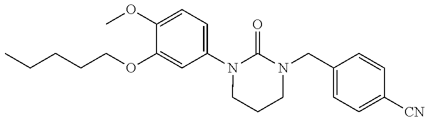 [SMILES: CCCCCOc1cc(N2CCCN(Cc3ccc(C#N)cc3)C2=O)ccc1OC] | 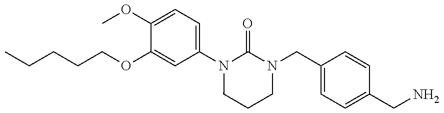 [SMILES: CCCCCOc1cc(N2CCCN(Cc3ccc(CN)cc3)C2=O)ccc1OC] |
| 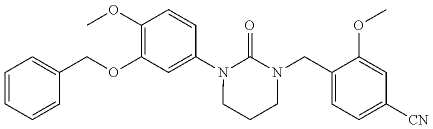 [SMILES: COc1cc(C#N)ccc1CN1CCCN(c2ccc(OC)c(OCc3ccccc3)c2)C1=O] | 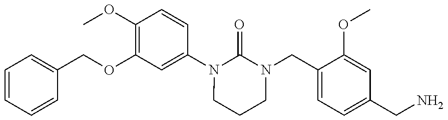 [SMILES: COc1cc(CN)ccc1CN1CCCN(c2ccc(OC)c(OCc3ccccc3)c2)C1=O] |
| 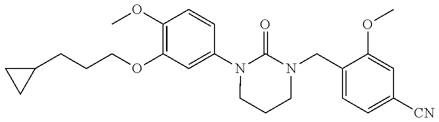 [SMILES: COc1cc(C#N)ccc1CN1CCCN(c2ccc(OC)c(OCCCC3CC3)c2)C1=O] | 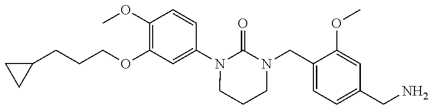 [SMILES: COc1cc(CN)ccc1CN1CCCN(c2ccc(OC)c(OCCCC3CC3)c2)C1=O] |
| 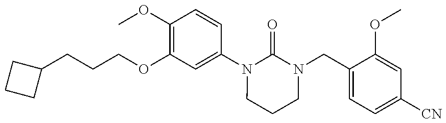 [SMILES: COc1cc(C#N)ccc1CN1CCCN(c2ccc(OC)c(OCCCC3CCC3)c2)C1=O] | 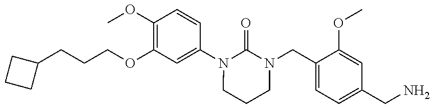 [SMILES: COc1cc(CN)ccc1CN1CCCN(c2ccc(OC)c(OCCCC3CCC3)c2)C1=O] |
| 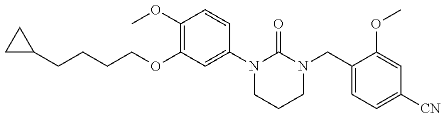 [SMILES: COc1cc(C#N)ccc1CN1CCCN(c2ccc(OC)c(OCCCCC3CC3)c2)C1=O] | 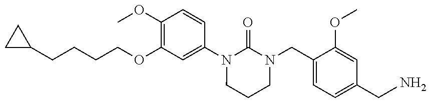 [SMILES: COc1cc(CN)ccc1CN1CCCN(c2ccc(OC)c(OCCCCC3CC3)c2)C1=O] |
| 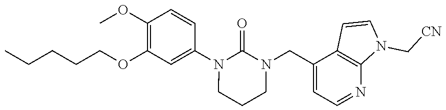 [SMILES: CCCCCOc1cc(N2CCCN(Cc3ccnc4c3ccn4CC#N)C2=O)ccc1OC] | 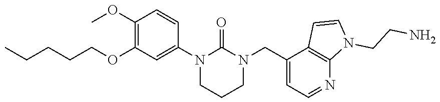 [SMILES: CCCCCOc1cc(N2CCCN(Cc3ccnc4c3ccn4CCN)C2=O)ccc1OC] |
| 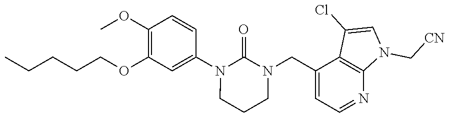 [SMILES: CCCCCOc1cc(N2CCCN(Cc3ccnc4c3c(Cl)cn4CC#N)C2=O)ccc1OC] | 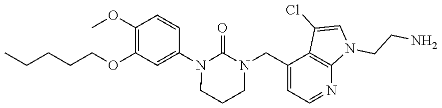 [SMILES: CCCCCOc1cc(N2CCCN(Cc3ccnc4c3c(Cl)cn4CCN)C2=O)ccc1OC] |
| 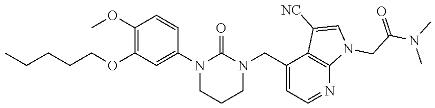 [SMILES: CCCCCOc1cc(N2CCCN(Cc3ccnc4c3c(C#N)cn4CC(=O)N(C)C)C2=O)ccc1OC] | 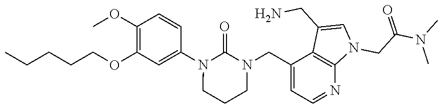 [SMILES: CCCCCOc1cc(N2CCCN(Cc3ccnc4c3c(CN)cn4CC(=O)N(C)C)C2=O)ccc1OC] |

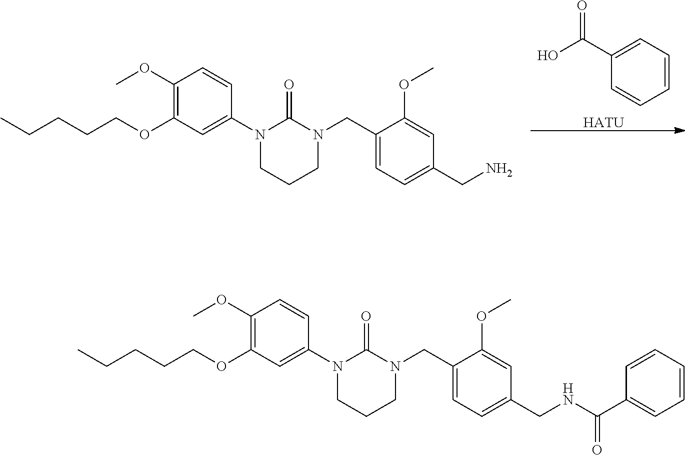

1-(4-(aminomethyl)-2-methoxybenzyl)-3-(4-methoxy-3-(pentyloxy)phenyl)tetrahydropyrimidin-2(1H)-one (50 mg, 0.11 mmol) is dissolved in N,N-dimethylformamide (1.1 mL), then N-[(dimethylamino)-1H-1,2,3-triazolo-[4,5-b]pyridin-1-ylmethylene]-N-methylmethanaminium hexafluorophosphate N-oxide (HATU, 52 mg, 0.14 mmol), benzoic acid (15 mg, 0.12 mmol), and triethylamine (23 uL, 0.17 mmol) are added. The reaction mixture is stirred at room temperature for 2 hours. Water is added, and the reaction mixture is extracted with ethyl acetate. The combined organic layers are dried over anhydrous sodium sulfate, filtered, and concentrated in vacuo. Purification by column chromatography over silica gel afforded N-(3-methoxy-4-((3-(4-methoxy-3-(pentyloxy)phenyl)-2-oxotetrahydropyrimidin-1(2H)-yl)methyl)benzyl)benzamide.

The following compounds are prepared in a similar manner as described above.

| Amine | Acid |
|---|---|
| 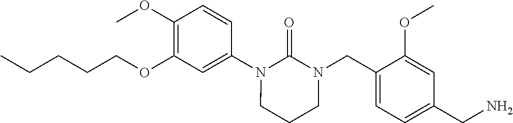 [SMILES: CCCCCOc1cc(N2CCCN(Cc3ccc(CN)cc3OC)C2=O)ccc1OC] | 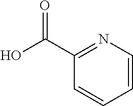 |
| 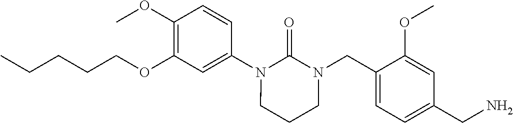 [SMILES: CCCCCOc1cc(N2CCCN(Cc3ccc(CN)cc3OC)C2=O)ccc1OC] | 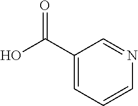 |
| 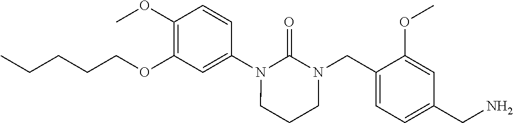 [SMILES: CCCCCOc1cc(N2CCCN(Cc3ccc(CN)cc3OC)C2=O)ccc1OC] | 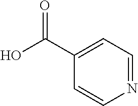 |
| 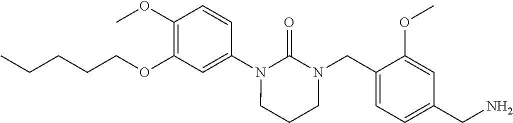 [SMILES: CCCCCOc1cc(N2CCCN(Cc3ccc(CN)cc3OC)C2=O)ccc1OC] | 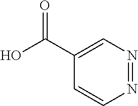 |
| 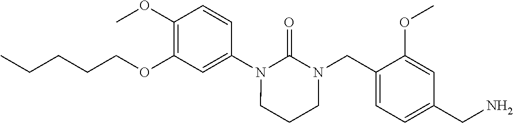 [SMILES: CCCCCOc1cc(N2CCCN(Cc3ccc(CN)cc3OC)C2=O)ccc1OC] | 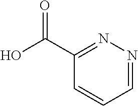 |
| 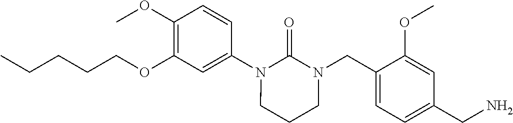 [SMILES: CCCCCOc1cc(N2CCCN(Cc3ccc(CN)cc3OC)C2=O)ccc1OC] | 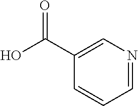 |
| 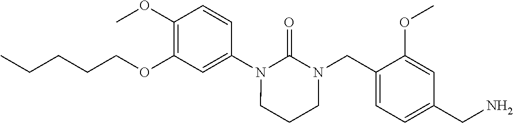 [SMILES: CCCCCOc1cc(N2CCCN(Cc3ccc(CN)cc3OC)C2=O)ccc1OC] | 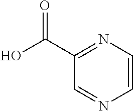 |
| 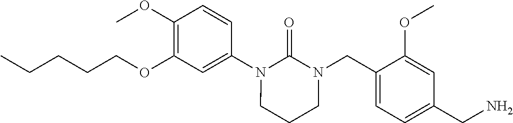 [SMILES: CCCCCOc1cc(N2CCCN(Cc3ccc(CN)cc3OC)C2=O)ccc1OC] | 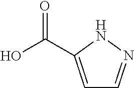 |
| 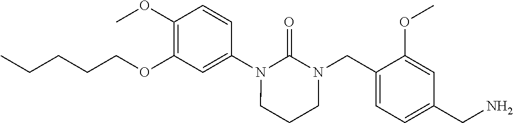 [SMILES: CCCCCOc1cc(N2CCCN(Cc3ccc(CN)cc3OC)C2=O)ccc1OC] | 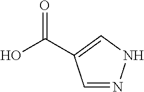 |
| 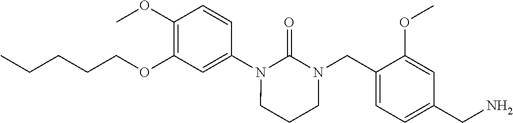 [SMILES: CCCCCOc1cc(N2CCCN(Cc3ccc(CN)cc3OC)C2=O)ccc1OC] | 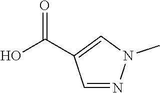 |
| 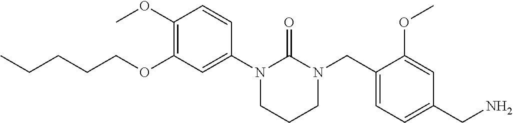 [SMILES: CCCCCOc1cc(N2CCCN(Cc3ccc(CN)cc3OC)C2=O)ccc1OC] | 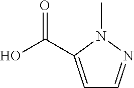 |
| 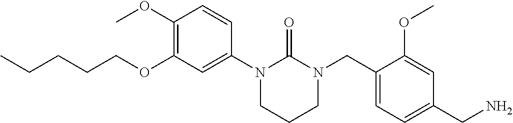 [SMILES: CCCCCOc1cc(N2CCCN(Cc3ccc(CN)cc3OC)C2=O)ccc1OC] | 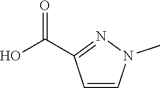 |
| 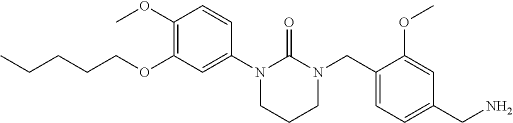 [SMILES: CCCCCOc1cc(N2CCCN(Cc3ccc(CN)cc3OC)C2=O)ccc1OC] | 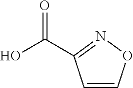 |
| 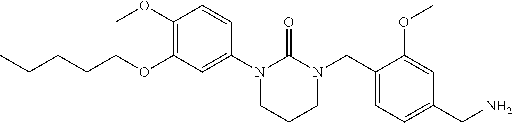 [SMILES: CCCCCOc1cc(N2CCCN(Cc3ccc(CN)cc3OC)C2=O)ccc1OC] | 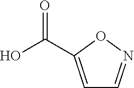 |
| 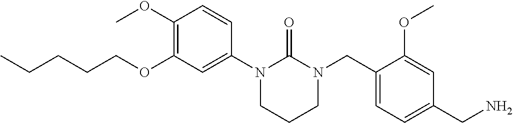 [SMILES: CCCCCOc1cc(N2CCCN(Cc3ccc(CN)cc3OC)C2=O)ccc1OC] | 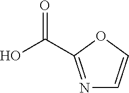 |
| 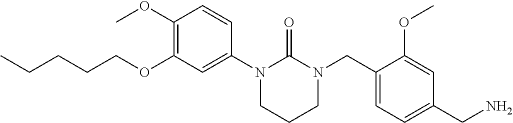 [SMILES: CCCCCOc1cc(N2CCCN(Cc3ccc(CN)cc3OC)C2=O)ccc1OC] | 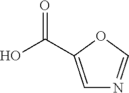 |
| 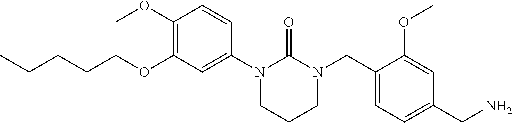 [SMILES: CCCCCOc1cc(N2CCCN(Cc3ccc(CN)cc3OC)C2=O)ccc1OC] | 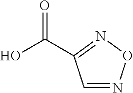 |
| 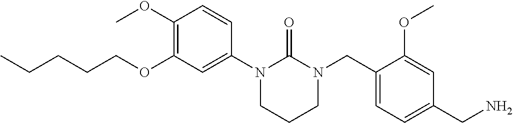 [SMILES: CCCCCOc1cc(N2CCCN(Cc3ccc(CN)cc3OC)C2=O)ccc1OC] | 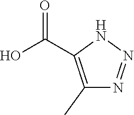 |
| 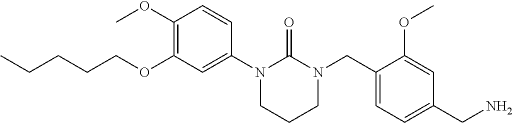 [SMILES: CCCCCOc1cc(N2CCCN(Cc3ccc(CN)cc3OC)C2=O)ccc1OC] | 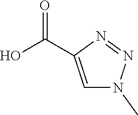 |
| 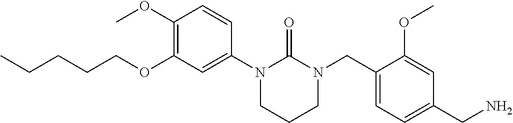 [SMILES: CCCCCOc1cc(N2CCCN(Cc3ccc(CN)cc3OC)C2=O)ccc1OC] | 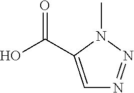 |
| 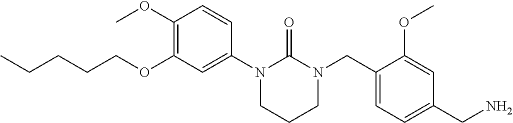 [SMILES: CCCCCOc1cc(N2CCCN(Cc3ccc(CN)cc3OC)C2=O)ccc1OC] | 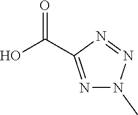 |
| 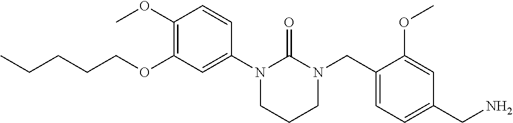 [SMILES: CCCCCOc1cc(N2CCCN(Cc3ccc(CN)cc3OC)C2=O)ccc1OC] | 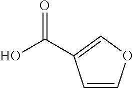 |
| 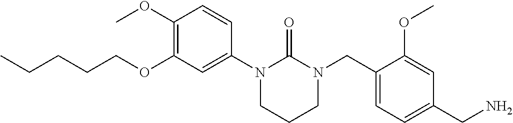 [SMILES: CCCCCOc1cc(N2CCCN(Cc3ccc(CN)cc3OC)C2=O)ccc1OC] | 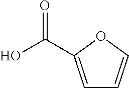 |
| 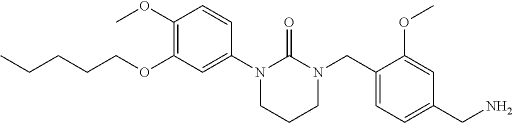 [SMILES: CCCCCOc1cc(N2CCCN(Cc3ccc(CN)cc3OC)C2=O)ccc1OC] | 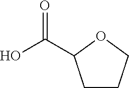 |
| 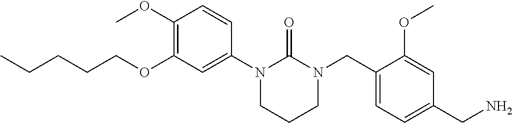 [SMILES: CCCCCOc1cc(N2CCCN(Cc3ccc(CN)cc3OC)C2=O)ccc1OC] | 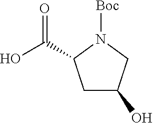 [SMILES: CC(C)(C)OC(=O)N1C[C@@H](O)C[C@@H]1C(=O)O] |
| 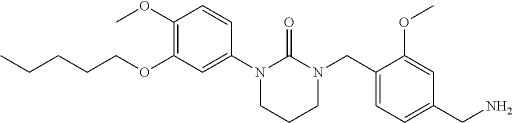 [SMILES: CCCCCOc1cc(N2CCCN(Cc3ccc(CN)cc3OC)C2=O)ccc1OC] | 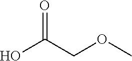 |
| 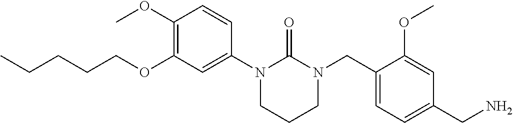 [SMILES: CCCCCOc1cc(N2CCCN(Cc3ccc(CN)cc3OC)C2=O)ccc1OC] | 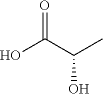 |
| 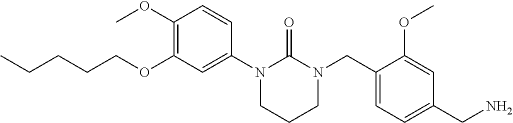 [SMILES: CCCCCOc1cc(N2CCCN(Cc3ccc(CN)cc3OC)C2=O)ccc1OC] | 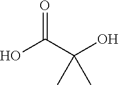 |
| 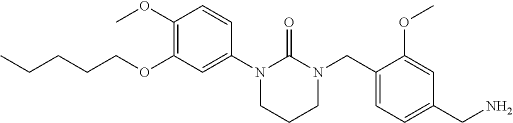 [SMILES: CCCCCOc1cc(N2CCCN(Cc3ccc(CN)cc3OC)C2=O)ccc1OC] | 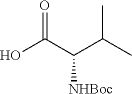 |
| 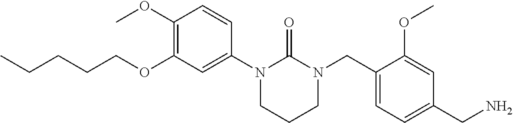 [SMILES: CCCCCOc1cc(N2CCCN(Cc3ccc(CN)cc3OC)C2=O)ccc1OC] | 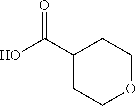 |
| 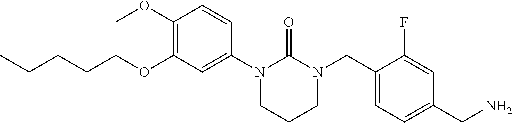 [SMILES: CCCCCOc1cc(N2CCCN(Cc3ccc(CN)cc3F)C2=O)ccc1OC] | 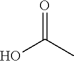 |
| 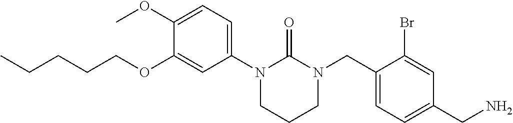 [SMILES: CCCCCOc1cc(N2CCCN(Cc3ccc(CN)cc3Br)C2=O)ccc1OC] | 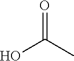 |
| 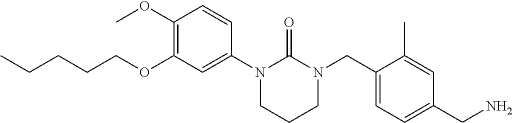 [SMILES: CCCCCOc1cc(N2CCCN(Cc3ccc(CN)cc3C)C2=O)ccc1OC] | 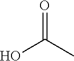 |
| 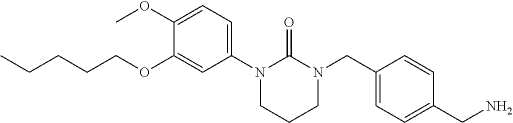 [SMILES: CCCCCOc1cc(N2CCCN(Cc3ccc(CN)cc3)C2=O)ccc1OC] | 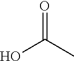 |
| 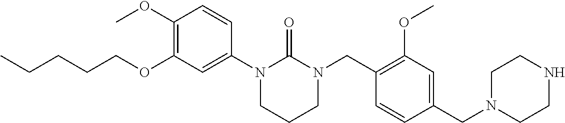 [SMILES: CCCCCOc1cc(N2CCCN(Cc3ccc(CN4CCNCC4)cc3OC)C2=O)ccc1OC] | 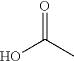 |
| 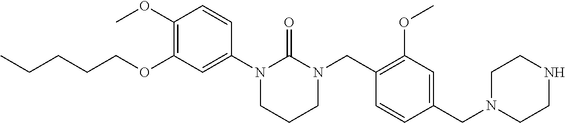 [SMILES: CCCCCOc1cc(N2CCCN(Cc3ccc(CN4CCNCC4)cc3OC)C2=O)ccc1OC] | 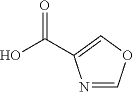 |
| 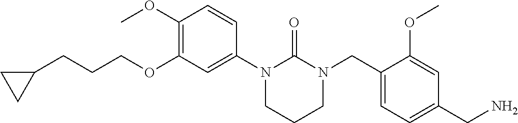 [SMILES: COc1cc(CN)ccc1CN1CCCN(c2ccc(OC)c(OCCCC3CC3)c2)C1=O] | 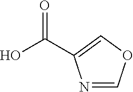 |
| 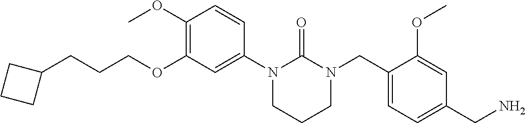 [SMILES: COc1cc(CN)ccc1CN1CCCN(c2ccc(OC)c(OCCCC3CCC3)c2)C1=O] | 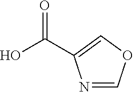 |
| 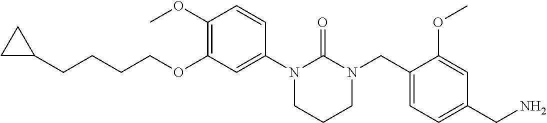 [SMILES: COc1cc(CN)ccc1CN1CCCN(c2ccc(OC)c(OCCCCC3CC3)c2)C1=O] | 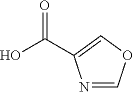 |
| 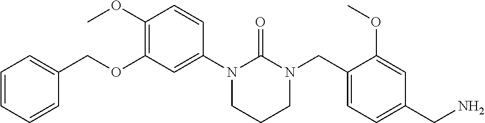 [SMILES: COc1cc(CN)ccc1CN1CCCN(c2ccc(OC)c(OCc3ccccc3)c2)C1=O] | 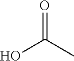 |
| 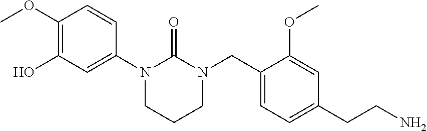 [SMILES: COc1ccc(N2CCCN(Cc3ccc(CCN)cc3OC)C2=O)cc1O] | 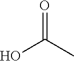 |
| 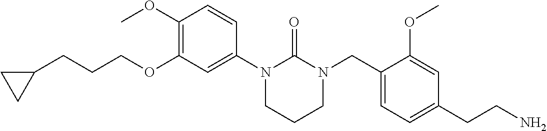 [SMILES: COc1cc(CCN)ccc1CN1CCCN(c2ccc(OC)c(OCCCC3CC3)c2)C1=O] | 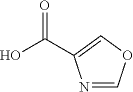 |
| 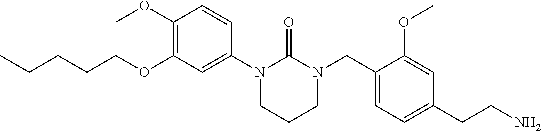 [SMILES: CCCCCOc1cc(N2CCCN(Cc3ccc(CCN)cc3OC)C2=O)ccc1OC] | 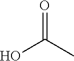 |
| 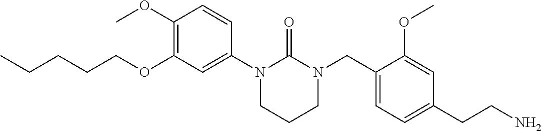 [SMILES: CCCCCOc1cc(N2CCCN(Cc3ccc(CCN)cc3OC)C2=O)ccc1OC] | 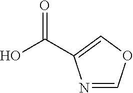 |
| 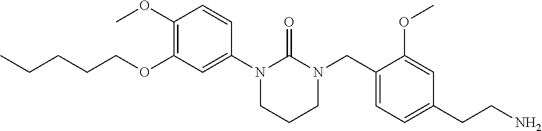 [SMILES: CCCCCOc1cc(N2CCCN(Cc3ccc(CCN)cc3OC)C2=O)ccc1OC] | 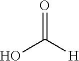 |
| 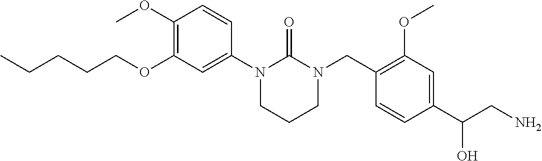 [SMILES: CCCCCOc1cc(N2CCCN(Cc3ccc(C(O)CN)cc3OC)C2=O)ccc1OC] | 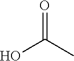 |
| 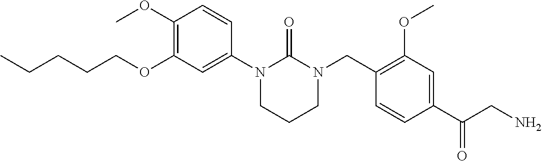 [SMILES: CCCCCOc1cc(N2CCCN(Cc3ccc(C(=O)CN)cc3OC)C2=O)ccc1OC] | 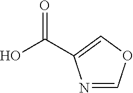 |
| 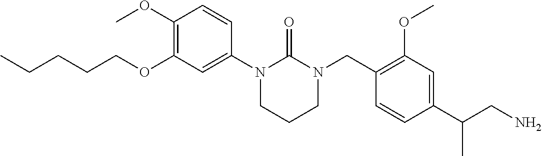 [SMILES: CCCCCOc1cc(N2CCCN(Cc3ccc(C(C)CN)cc3OC)C2=O)ccc1OC] | 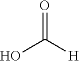 |
| 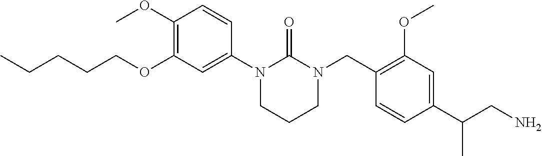 [SMILES: CCCCCOc1cc(N2CCCN(Cc3ccc(C(C)CN)cc3OC)C2=O)ccc1OC] | 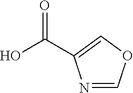 |
| 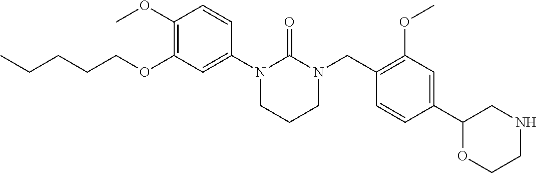 [SMILES: CCCCCOc1cc(N2CCCN(Cc3ccc(C4CNCCO4)cc3OC)C2=O)ccc1OC] | 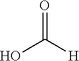 |
| 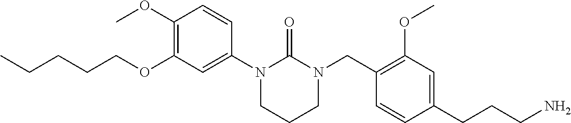 [SMILES: CCCCCOc1cc(N2CCCN(Cc3ccc(CCCN)cc3OC)C2=O)ccc1OC] | 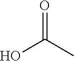 |
| 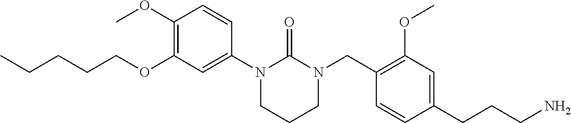 [SMILES: CCCCCOc1cc(N2CCCN(Cc3ccc(CCCN)cc3OC)C2=O)ccc1OC] | 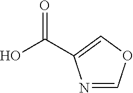 |
| 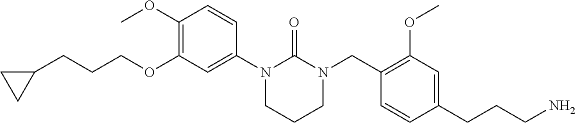 [SMILES: COc1cc(CCCN)ccc1CN1CCCN(c2ccc(OC)c(OCCCC3CC3)c2)C1=O] | 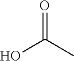 |
| 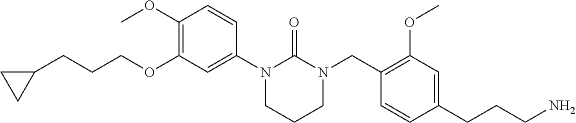 [SMILES: COc1cc(CCCN)ccc1CN1CCCN(c2ccc(OC)c(OCCCC3CC3)c2)C1=O] | 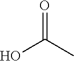 |
| 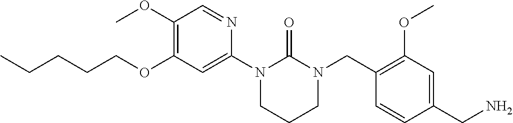 [SMILES: CCCCCOc1cc(N2CCCN(Cc3ccc(CN)cc3OC)C2=O)ncc1OC] | 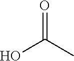 |
| 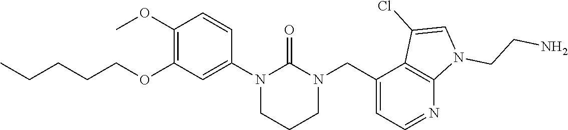 [SMILES: CCCCCOc1cc(N2CCCN(Cc3ccnc4c3c(Cl)cn4CCN)C2=O)ccc1OC] | 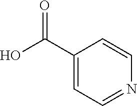 |
| 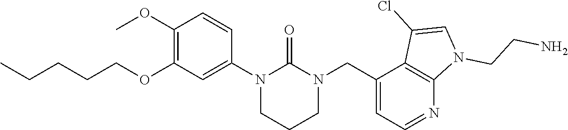 [SMILES: CCCCCOc1cc(N2CCCN(Cc3ccnc4c3c(Cl)cn4CCN)C2=O)ccc1OC] | 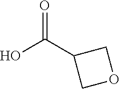 |
| 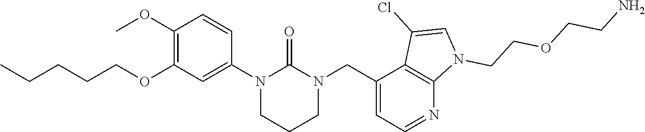 [SMILES: CCCCCOc1cc(N2CCCN(Cc3ccnc4c3c(Cl)cn4CCOCCN)C2=O)ccc1OC] | 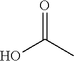 |

Compound

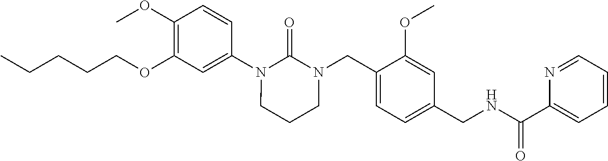 [SMILES: CCCCCOc1cc(N2CCCN(Cc3ccc(CNC(=O)c4ccccn4)cc3OC)C2=O)ccc1OC]

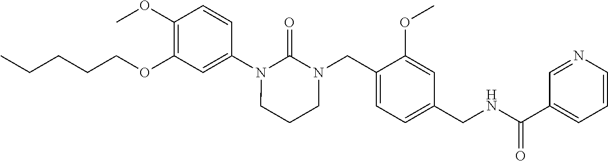 [SMILES: CCCCCOc1cc(N2CCCN(Cc3ccc(CNC(=O)c4cccnc4)cc3OC)C2=O)ccc1OC]

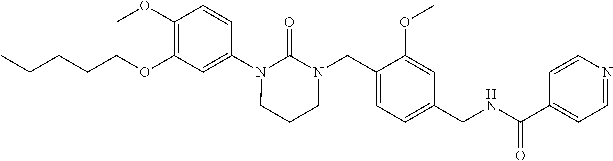 [SMILES: CCCCCOc1cc(N2CCCN(Cc3ccc(CNC(=O)c4ccncc4)cc3OC)C2=O)ccc1OC]

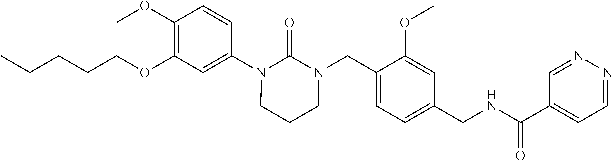 [SMILES: CCCCCOc1cc(N2CCCN(Cc3ccc(CNC(=O)c4ccnnc4)cc3OC)C2=O)ccc1OC]

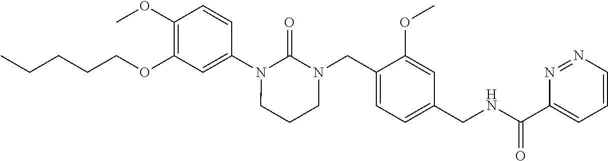 [SMILES: CCCCCOc1cc(N2CCCN(Cc3ccc(CNC(=O)c4cccnn4)cc3OC)C2=O)ccc1OC]

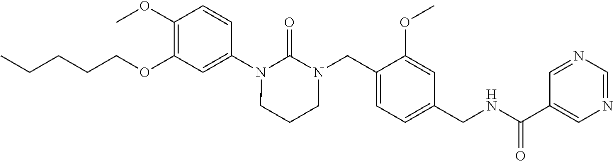 [SMILES: CCCCCOc1cc(N2CCCN(Cc3ccc(CNC(=O)c4cncnc4)cc3OC)C2=O)ccc1OC]

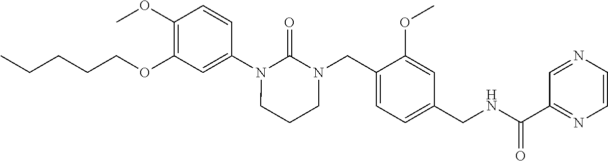 [SMILES: CCCCCOc1cc(N2CCCN(Cc3ccc(CNC(=O)c4cnccn4)cc3OC)C2=O)ccc1OC]

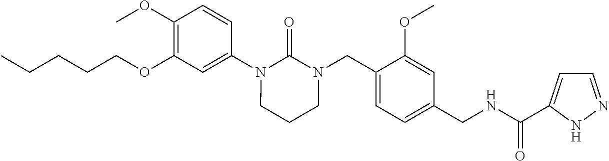 [SMILES: CCCCCOc1cc(N2CCCN(Cc3ccc(CNC(=O)c4ccn[nH]4)cc3OC)C2=O)ccc1OC]

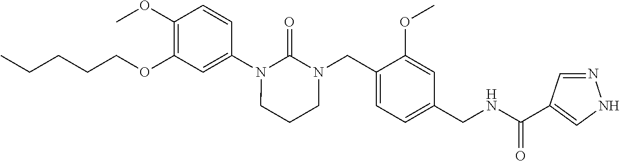 [SMILES: CCCCCOc1cc(N2CCCN(Cc3ccc(CNC(=O)c4cn[nH]c4)cc3OC)C2=O)ccc1OC]

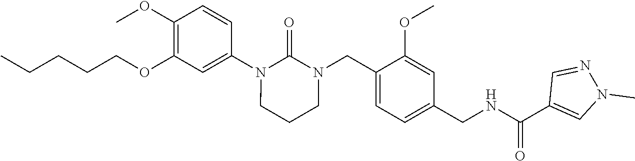 [SMILES: CCCCCOc1cc(N2CCCN(Cc3ccc(CNC(=O)c4cnn(C)c4)cc3OC)C2=O)ccc1OC]

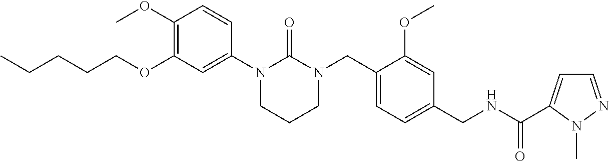 [SMILES: CCCCCOc1cc(N2CCCN(Cc3ccc(CNC(=O)c4ccnn4C)cc3OC)C2=O)ccc1OC]

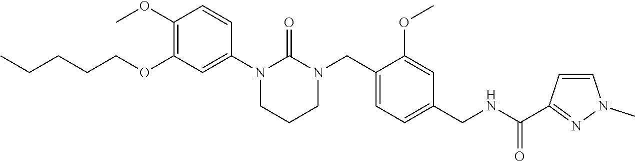 [SMILES: CCCCCOc1cc(N2CCCN(Cc3ccc(CNC(=O)c4ccn(C)n4)cc3OC)C2=O)ccc1OC]

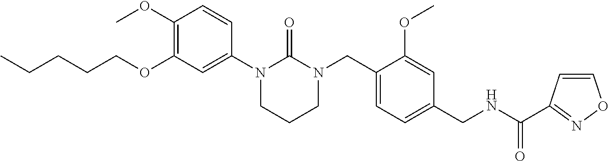 [SMILES: CCCCCOc1cc(N2CCCN(Cc3ccc(CNC(=O)c4ccon4)cc3OC)C2=O)ccc1OC]

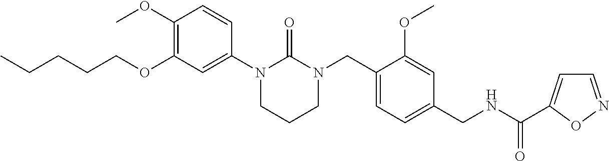 [SMILES: CCCCCOc1cc(N2CCCN(Cc3ccc(CNC(=O)c4ccno4)cc3OC)C2=O)ccc1OC]

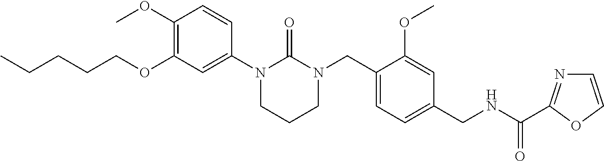 [SMILES: CCCCCOc1cc(N2CCCN(Cc3ccc(CNC(=O)c4ncco4)cc3OC)C2=O)ccc1OC]

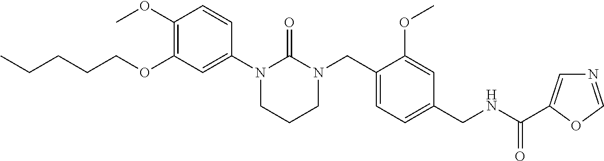 [SMILES: CCCCCOc1cc(N2CCCN(Cc3ccc(CNC(=O)c4cnco4)cc3OC)C2=O)ccc1OC]

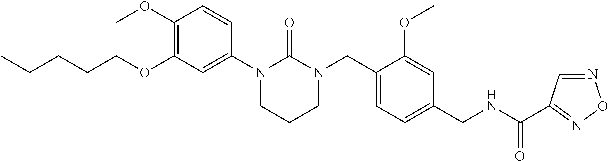 [SMILES: CCCCCOc1cc(N2CCCN(Cc3ccc(CNC(=O)c4cnon4)cc3OC)C2=O)ccc1OC]

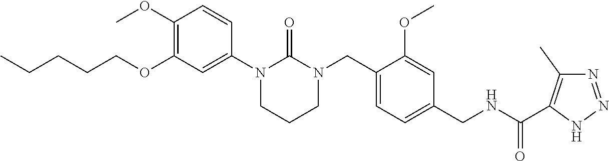 [SMILES: CCCCCOc1cc(N2CCCN(Cc3ccc(CNC(=O)c4[nH]nnc4C)cc3OC)C2=O)ccc1OC]

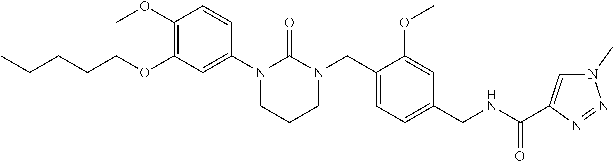 [SMILES: CCCCCOc1cc(N2CCCN(Cc3ccc(CNC(=O)c4cn(C)nn4)cc3OC)C2=O)ccc1OC]

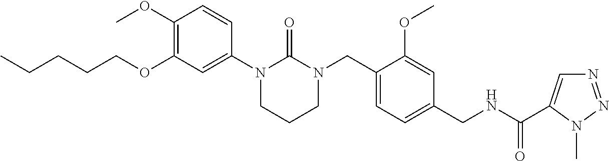 [SMILES: CCCCCOc1cc(N2CCCN(Cc3ccc(CNC(=O)c4cnnn4C)cc3OC)C2=O)ccc1OC]

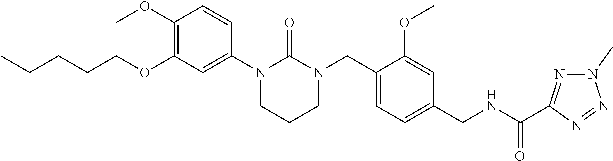 [SMILES: CCCCCOc1cc(N2CCCN(Cc3ccc(CNC(=O)c4nnn(C)n4)cc3OC)C2=O)ccc1OC]

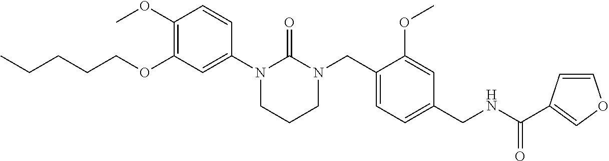 [SMILES: CCCCCOc1cc(N2CCCN(Cc3ccc(CNC(=O)c4ccoc4)cc3OC)C2=O)ccc1OC]

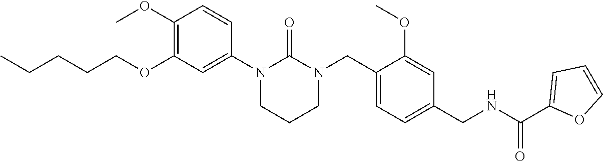 [SMILES: CCCCCOc1cc(N2CCCN(Cc3ccc(CNC(=O)c4ccco4)cc3OC)C2=O)ccc1OC]

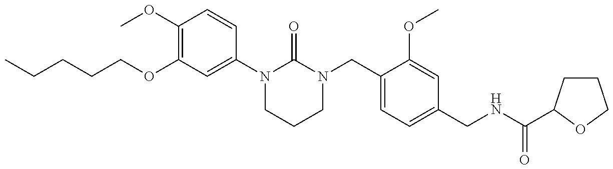 [SMILES: CCCCCOc1cc(N2CCCN(Cc3ccc(CNC(=O)C4CCCO4)cc3OC)C2=O)ccc1OC]

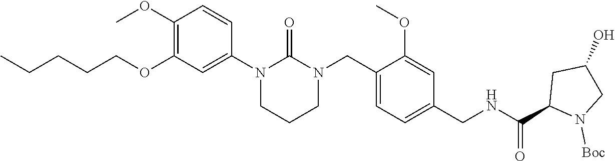 [SMILES: CCCCCOc1cc(N2CCCN(Cc3ccc(CNC(=O)[C@H]4C[C@H](O)CN4C(=O)OC(C)(C)C)cc3OC)C2=O)ccc1OC]

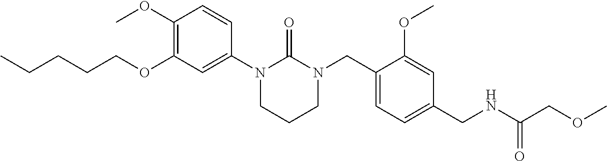 [SMILES: CCCCCOc1cc(N2CCCN(Cc3ccc(CNC(=O)COC)cc3OC)C2=O)ccc1OC]

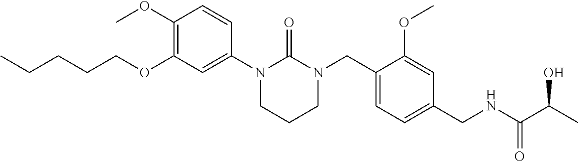 [SMILES: CCCCCOc1cc(N2CCCN(Cc3ccc(CNC(=O)[C@H](C)O)cc3OC)C2=O)ccc1OC]

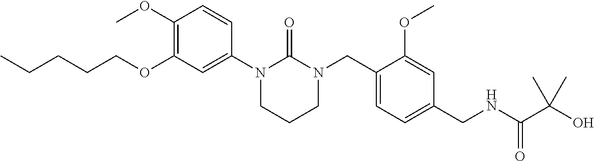 [SMILES: CCCCCOc1cc(N2CCCN(Cc3ccc(CNC(=O)C(C)(C)O)cc3OC)C2=O)ccc1OC]

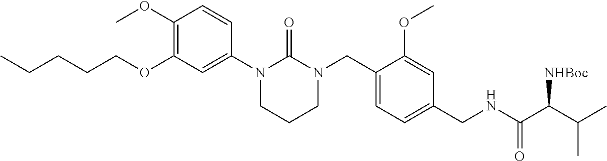 [SMILES: CCCCCOc1cc(N2CCCN(Cc3ccc(CNC(=O)[C@@H](NC(=O)OC(C)(C)C)C(C)C)cc3OC)C2=O)ccc1OC]

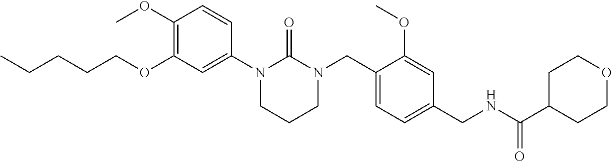 [SMILES: CCCCCOc1cc(N2CCCN(Cc3ccc(CNC(=O)C4CCOCC4)cc3OC)C2=O)ccc1OC]

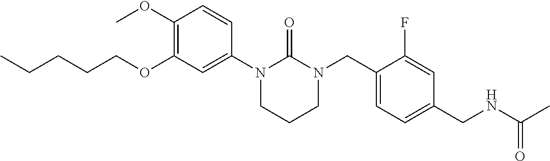 [SMILES: CCCCCOc1cc(N2CCCN(Cc3ccc(CNC(C)=O)cc3F)C2=O)ccc1OC]

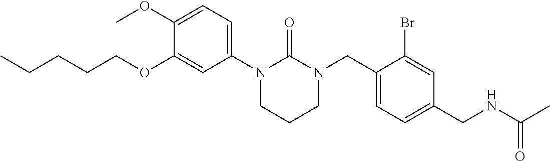 [SMILES: CCCCCOc1cc(N2CCCN(Cc3ccc(CNC(C)=O)cc3Br)C2=O)ccc1OC]

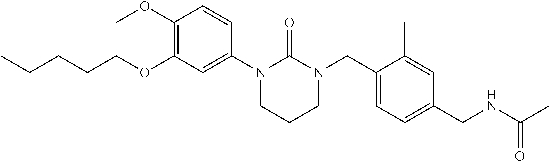 [SMILES: CCCCCOc1cc(N2CCCN(Cc3ccc(CNC(C)=O)cc3C)C2=O)ccc1OC]

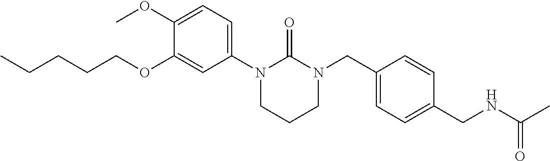 [SMILES: CCCCCOc1cc(N2CCCN(Cc3ccc(CNC(C)=O)cc3)C2=O)ccc1OC]

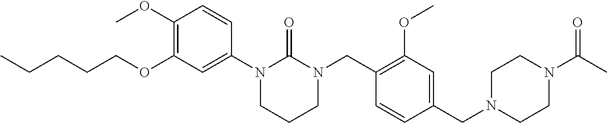 [SMILES: CCCCCOc1cc(N2CCCN(Cc3ccc(CN4CCN(C(C)=O)CC4)cc3OC)C2=O)ccc1OC]

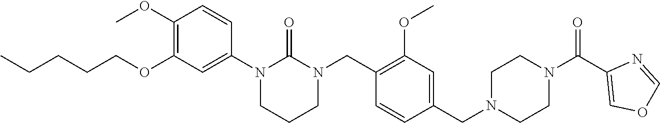 [SMILES: CCCCCOc1cc(N2CCCN(Cc3ccc(CN4CCN(C(=O)c5cocn5)CC4)cc3OC)C2=O)ccc1OC]

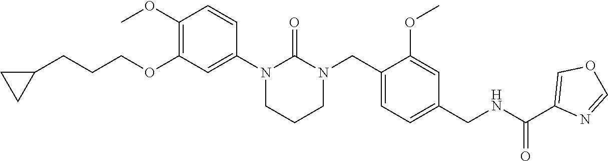 [SMILES: COc1cc(CNC(=O)c2cocn2)ccc1CN1CCCN(c2ccc(OC)c(OCCCC3CC3)c2)C1=O]

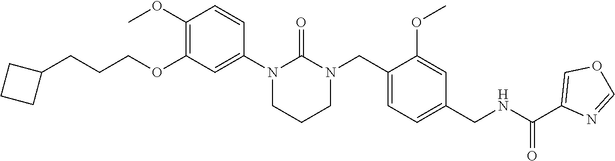 [SMILES: COc1cc(CNC(=O)c2cocn2)ccc1CN1CCCN(c2ccc(OC)c(OCCCC3CCC3)c2)C1=O]

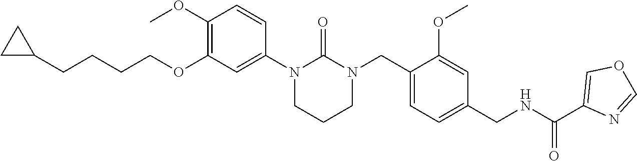 [SMILES: COc1cc(CNC(=O)c2cocn2)ccc1CN1CCCN(c2ccc(OC)c(OCCCCC3CC3)c2)C1=O]

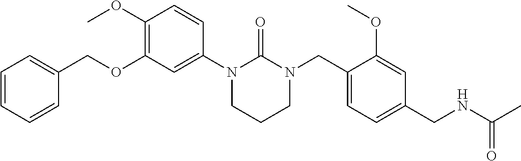 [SMILES: COc1cc(CNC(C)=O)ccc1CN1CCCN(c2ccc(OC)c(OCc3ccccc3)c2)C1=O]

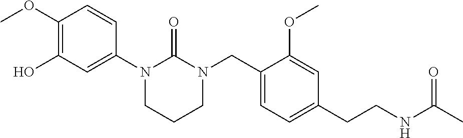 [SMILES: COc1ccc(N2CCCN(Cc3ccc(CCNC(C)=O)cc3OC)C2=O)cc1O]

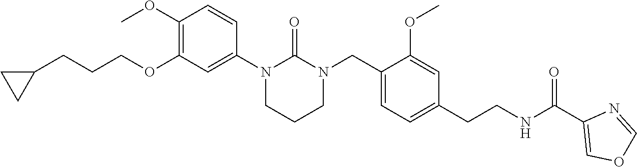 [SMILES: COc1cc(CCNC(=O)c2cocn2)ccc1CN1CCCN(c2ccc(OC)c(OCCCC3CC3)c2)C1=O]

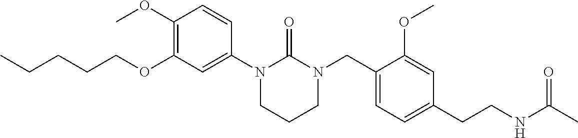 [SMILES: CCCCCOc1cc(N2CCCN(Cc3ccc(CCNC(C)=O)cc3OC)C2=O)ccc1OC]

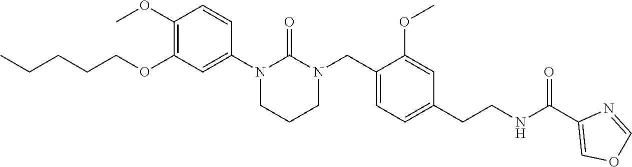 [SMILES: CCCCCOc1cc(N2CCCN(Cc3ccc(CCNC(=O)c4cocn4)cc3OC)C2=O)ccc1OC]

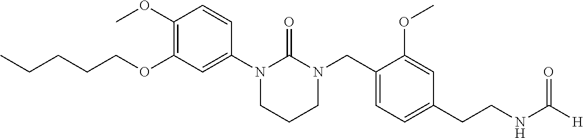 [SMILES: CCCCCOc1cc(N2CCCN(Cc3ccc(CCNC=O)cc3OC)C2=O)ccc1OC]

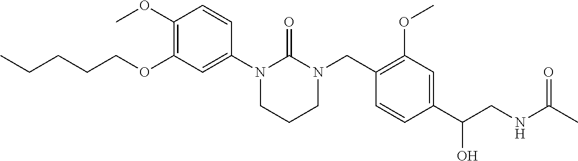 [SMILES: CCCCCOc1cc(N2CCCN(Cc3ccc(C(O)CNC(C)=O)cc3OC)C2=O)ccc1OC]

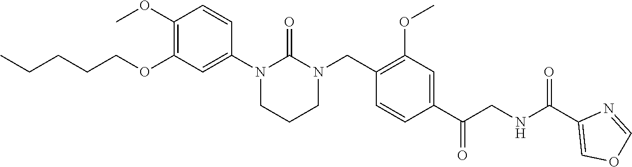 [SMILES: CCCCCOc1cc(N2CCCN(Cc3ccc(C(=O)CNC(=O)c4cocn4)cc3OC)C2=O)ccc1OC]

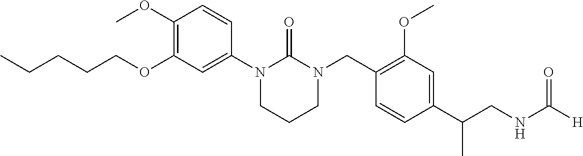 [SMILES: CCCCCOc1cc(N2CCCN(Cc3ccc(C(C)CNC=O)cc3OC)C2=O)ccc1OC]

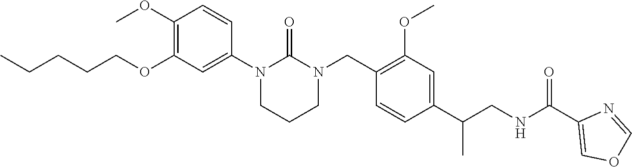 [SMILES: CCCCCOc1cc(N2CCCN(Cc3ccc(C(C)CNC(=O)c4cocn4)cc3OC)C2=O)ccc1OC]

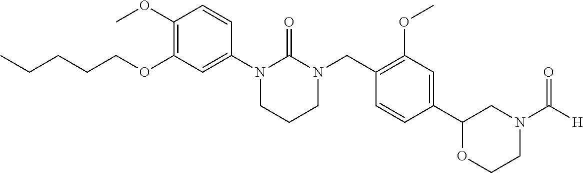 [SMILES: CCCCCOc1cc(N2CCCN(Cc3ccc(C4CN(C=O)CCO4)cc3OC)C2=O)ccc1OC]

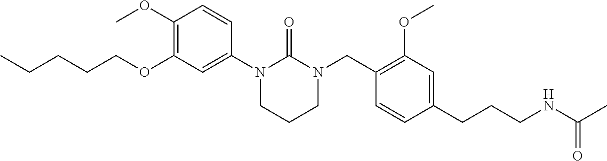 [SMILES: CCCCCOc1cc(N2CCCN(Cc3ccc(CCCNC(C)=O)cc3OC)C2=O)ccc1OC]

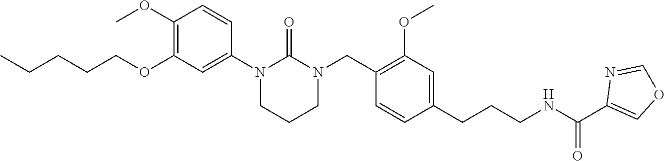 [SMILES: CCCCCOc1cc(N2CCCN(Cc3ccc(CCCNC(=O)c4cocn4)cc3OC)C2=O)ccc1OC]

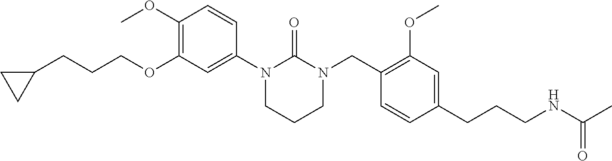 [SMILES: COc1cc(CCCNC(C)=O)ccc1CN1CCCN(c2ccc(OC)c(OCCCC3CC3)c2)C1=O]

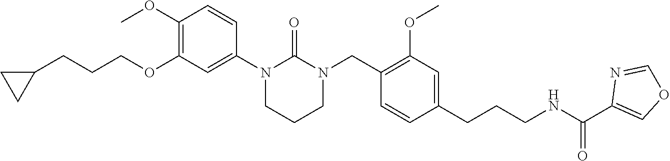 [SMILES: COc1cc(CCCNC(=O)c2cocn2)ccc1CN1CCCN(c2ccc(OC)c(OCCCC3CC3)c2)C1=O]

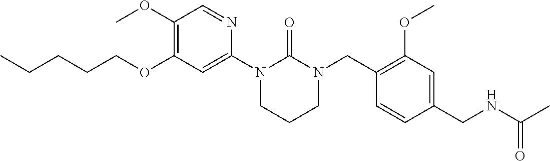 [SMILES: CCCCCOc1cc(N2CCCN(Cc3ccc(CNC(C)=O)cc3OC)C2=O)ncc1OC]

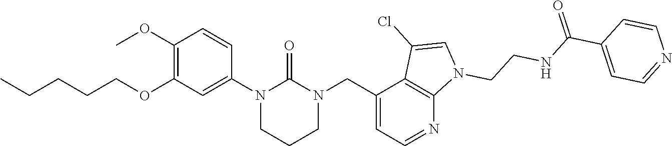 [SMILES: CCCCCOc1cc(N2CCCN(Cc3ccnc4c3c(Cl)cn4CCNC(=O)c3ccncc3)C2=O)ccc1OC]

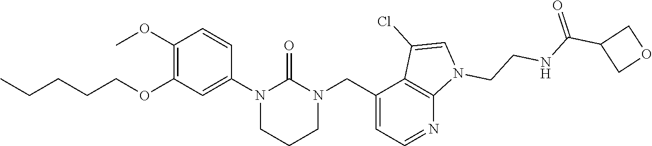 [SMILES: CCCCCOc1cc(N2CCCN(Cc3ccnc4c3c(Cl)cn4CCNC(=O)C3COC3)C2=O)ccc1OC]

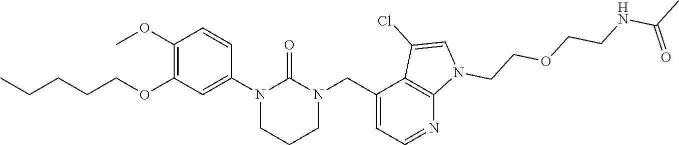 [SMILES: CCCCCOc1cc(N2CCCN(Cc3ccnc4c3c(Cl)cn4CCOCCNC(C)=O)C2=O)ccc1OC]

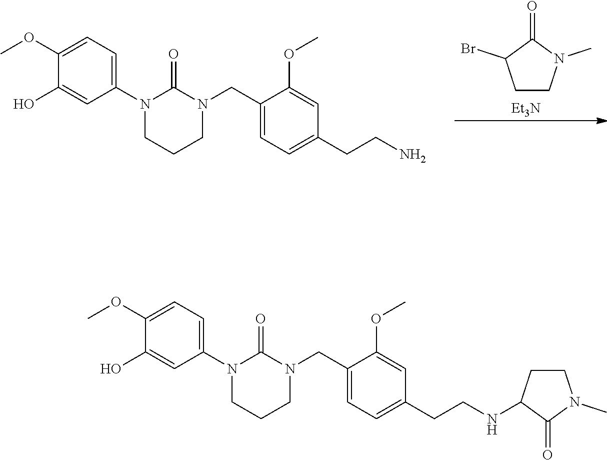

In a vial containing 1-(4-(2-aminoethyl)-2-methoxybenzyl)-3-(3-hydroxy-4-methoxyphenyl)tetrahydropyrimidin-2(1H)-one (100 mg, 0.026 mmol) are added N,N-dimethylformamide (2.6 mL), 3-bromo-1-methylpyrrolidin-2-one (69 mg, 0.039 mmol) and then triethylamine (110 uL, 0.78 mmol). The reaction is stirred overnight at room temperature. More 3-bromo-1-methylpyrrolidin-2-one (69 mg, 0.39 mmol) and triethylamine (110 uL, 0.78 umol) are added. The reaction is stirred at room temperature for another 24 hours. After diluting with water, the reaction is extracted with dichloromethane. The combined organic phases are washed with brine, then dried over anhydrous sodium sulfate, filtered, and concentrated in vacuo to afford 1-(3-hydroxy-4-methoxyphenyl)-3-(2-methoxy-4-(2-((1-methyl-2-oxopyrrolidin-3-yl)amino)ethyl)benzyl)tetrahydropyrimidin-2(1H)-one.

The following compounds are prepared in a similar manner as described above.

| | Alkyl |
|---|---|
| Amine | halide |
| 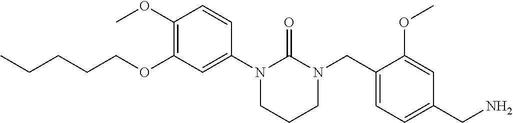 [SMILES: CCCCCOc1cc(N2CCCN(Cc3ccc(CN)cc3OC)C2=O)ccc1OC] | 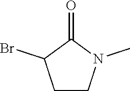 |
| 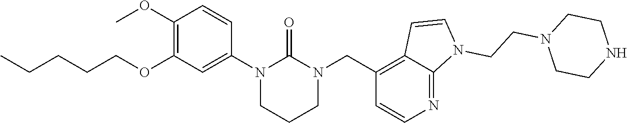 [SMILES: CCCCCOc1cc(N2CCCN(Cc3ccnc4c3ccn4CCN3CCNCC3)C2=O)ccc1OC] | 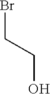 |
| 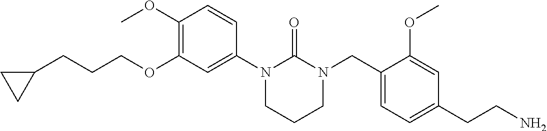 [SMILES: COc1cc(CCN)ccc1CN1CCCN(c2ccc(OC)c(OCCCC3CC3)c2)C1=O] | 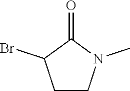 |

Compound

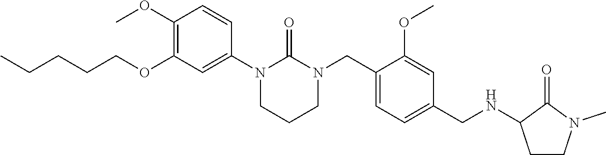 [SMILES: CCCCCOc1cc(N2CCCN(Cc3ccc(CNC4CCN(C)C4=O)cc3OC)C2=O)ccc1OC]

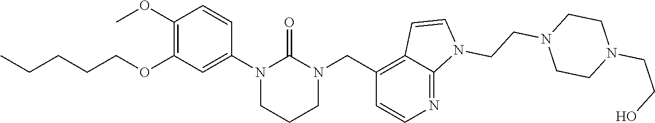 [SMILES: CCCCCOc1cc(N2CCCN(Cc3ccnc4c3ccn4CCN3CCN(CCO)CC3)C2=O)ccc1OC]

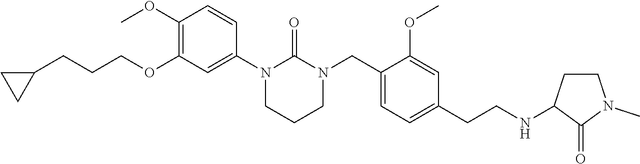 [SMILES: COc1cc(CCNC2CCN(C)C2=O)ccc1CN1CCCN(c2ccc(OC)c(OCCCC3CC3)c2)C1=O]

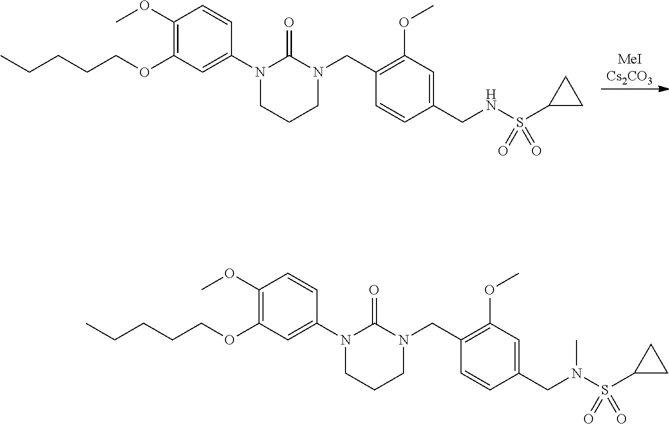 [SMILES: "MeICs2CO3" ; CCCCCOc1cc(N2CCCN(Cc3ccc(CNS(=O)(=O)C4CC4)cc3OC)C2=O)ccc1OC ; CCCCCOc1cc(N2CCCN(Cc3ccc(CN(C)S(=O)(=O)C4CC4)cc3OC)C2=O)ccc1OC]

A solution of N-(3-methoxy-4-((3 (4-methoxy-3-(pentyloxy)phenyl)-2-oxotetrahydropyrimidin-1(2H)-yl)methyl)benzyl)cyclopropanesulfonamide (25 mg, 0.046 mmol) in anhydrous N,N-dimethylformamide (0.5 mL) is treated with anhydrous cesium carbonate (33 mg, 0.10 mmol) and iodomethane (3 uL, 0.05 mmol). The reaction is stirred at room temperature for 6 hours. The reaction is diluted with water and extracted with ethyl acetate. The combined organic layers are dried over anhydrous sodium sulfate, filtered, and concentrated in vacuo. Purification by column chromatography over silica gel afforded N-(3-methoxy-4-((3-(4-methoxy-3-(pentyloxy)phenyl)-2-oxotetrahydropyrimidin-1(2H)-yl)methyl)benzyl)-N-methylcyclopropanesulfonamide.

The following compounds are prepared in a similar manner as described above:

| Sulfonamide | Halide |
|---|---|
| 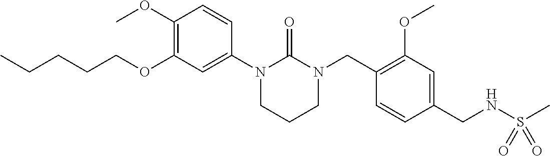 [SMILES: CCCCCOc1cc(N2CCCN(Cc3ccc(CNS(C)(=O)=O)cc3OC)C2=O)ccc1OC] | 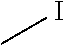 |
| 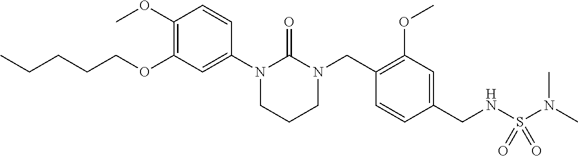 [SMILES: CCCCCOc1cc(N2CCCN(Cc3ccc(CNS(=O)(=O)N(C)C)cc3OC)C2=O)ccc1OC] | 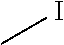 |
| 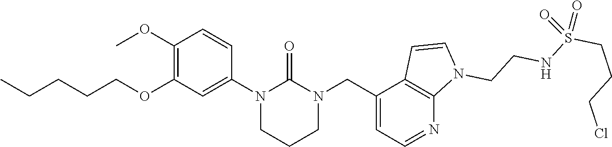 [SMILES: CCCCCOc1cc(N2CCCN(Cc3ccnc4c3ccn4CCNS(=O)(=O)CCCCl)C2=O)ccc1OC] | None |

Compound

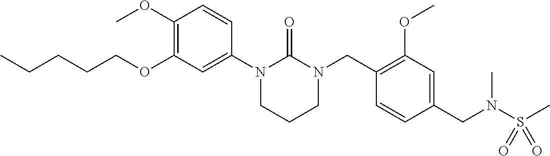 [SMILES: CCCCCOc1cc(N2CCCN(Cc3ccc(CN(C)S(C)(=O)=O)cc3OC)C2=O)ccc1OC]

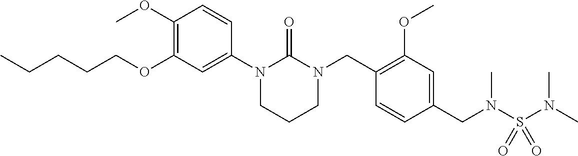 [SMILES: CCCCCOc1cc(N2CCCN(Cc3ccc(CN(C)S(=O)(=O)N(C)C)cc3OC)C2=O)ccc1OC]

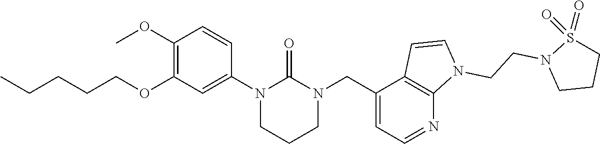 [SMILES: CCCCCOc1cc(N2CCCN(Cc3ccnc4c3ccn4CCN3CCCS3(=O)=O)C2=O)ccc1OC]

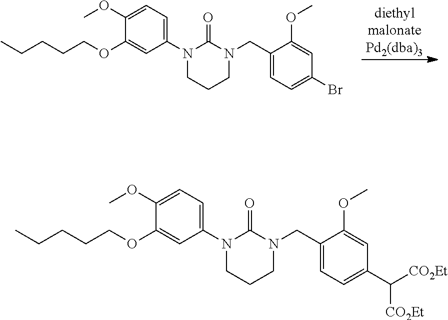 [SMILES: CCCCCOc1cc(N2CCCN(Cc3ccc(Br)cc3OC)C2=O)ccc1OC ; "diethylmalonatePd2(dba)3" ; CCCCCOc1cc(N2CCCN(Cc3ccc(C(C(=O)OCC)C(=O)OCC)cc3OC)C2=O)ccc1OC]

Cesium carbonate (16.9 g, 68 mmol) is added to a solution of 1-(4-bromo-2-methoxybenzyl)-3-(4-methoxy-3-(pentyloxy)phenyl) tetrahydropyrimidin-2(1H)-one (17.0 g, 34 mmol) and diethyl malonate (10.9 g, 68 mmol) in anhydrous N,N-dimethylformamide (150 mL). The mixture is sparged with dry nitrogen for 5 min, then tris(dibenzylideneacetone)dipalladium(0) (1.0 g, 1.1 mmol) and 2-dicyclohexylphosphino-2′,6′-dimethoxy-1,1′-biphenyl (SPhos, 1.0 g, 2.44 mmol) are added. The mixture is heated to 95° C. and is stirred at this temperature for 12 hours. After cooling to room temperature, the mixture is quenched with water then extracted with ethyl acetate. The combined organic phases are washed with brine, dried over anhydrous sodium sulfate, filtered, and concentrated in vacuo. Purification by column chromatography over silica gel afforded diethyl 2-(3-methoxy-4-((3-(4-methoxy-3-(pentyloxy)phenyl)-2-oxotetrahydropyrimidin-1(2H)-yl)methyl)phenyl)malonate.

The following compounds are prepared in a similar manner as described above.

| Bromide | Compound |
|---|---|
| 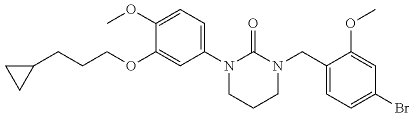 [SMILES: COc1cc(Br)ccc1CN1CCCN(c2ccc(OC)c(OCCCC3CC3)c2)C1=O] | 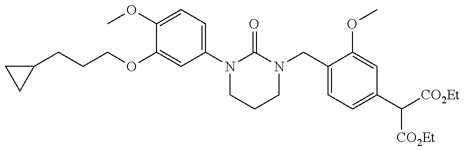 [SMILES: CCOC(=O)C(C(=O)OCC)c1ccc(CN2CCCN(c3ccc(OC)c(OCCCC4CC4)c3)C2=O)c(OC)c1] |
| 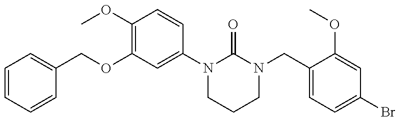 [SMILES: COc1cc(Br)ccc1CN1CCCN(c2ccc(OC)c(OCc3ccccc3)c2)C1=O] | 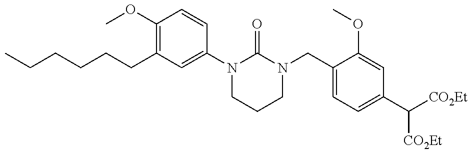 [SMILES: CCCCCCc1cc(N2CCCN(Cc3ccc(C(C(=O)OCC)C(=O)OCC)cc3OC)C2=O)ccc1OC] |
| 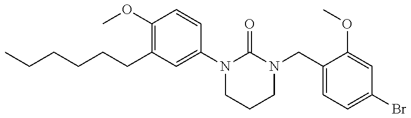 [SMILES: CCCCCCc1cc(N2CCCN(Cc3ccc(Br)cc3OC)C2=O)ccc1OC] | 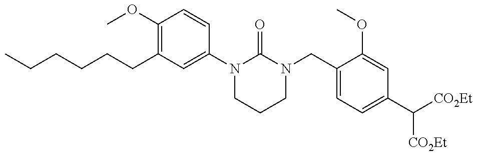 [SMILES: CCCCCCc1cc(N2CCCN(Cc3ccc(C(C(=O)OCC)C(=O)OCC)cc3OC)C2=O)ccc1OC] |
| 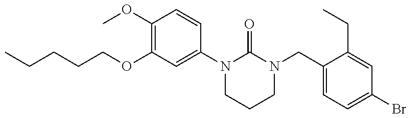 [SMILES: CCCCCOc1cc(N2CCCN(Cc3ccc(Br)cc3CC)C2=O)ccc1OC] | 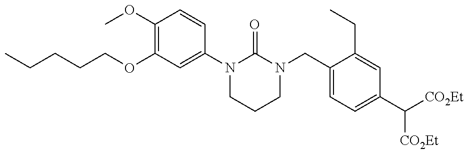 [SMILES: CCCCCOc1cc(N2CCCN(Cc3ccc(C(C(=O)OCC)C(=O)OCC)cc3CC)C2=O)ccc1OC] |

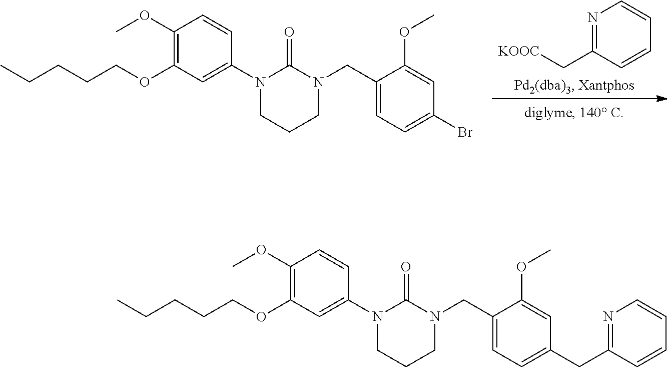

A 2-mL vial is charged with 1-(4-bromo-2-methoxybenzyl)-3-(4-methoxy-3-(pentyloxy)phenyl)tetrahydropyrimidin-2(1H)-one (100 mg, 0.20 mmol), potassium 2-(pyridin-2-yl)acetate (43 mg, 0.24 mmol), 4,4-bis(diphenylphosphino)-9,9-dimethylxanthene (Xantphos, 7 mg, 0.01 mmol) and tris(dibenzylideneacetone)dipalladium(0) (4 mg, 0.004 mmol). The vial is evacuated and purged with nitrogen three times. Diglyme (407 μL) is added, then the reaction was heated to 150° C. and stirred at this temperature for 24 hours. The reaction is cooled to room temperature, diluted with ethyl acetate, and filtered through CELITE®. The filter cake is washed with ethyl acetate. The filtered solution is washed with water then brine before it is dried over anhydrous sodium sulfate, filtered, and concentrated in vacuo. Purification by column chromatography over reversed-phase C18 silica gel afforded 1-(4-methoxy-3-(pentyloxy)phenyl)-3-(2-methoxy-4-(pyridin-2-ylmethyl)benzyl)tetrahydropyrimidin-2(1H)-one.

The following compounds are prepared in a similar manner as described above.

| Bromide | Heterocycle |
|---|---|
| 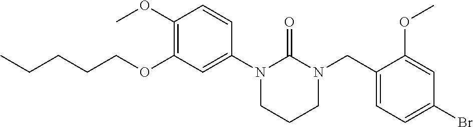 [SMILES: CCCCCOc1cc(N2CCCN(Cc3ccc(Br)cc3OC)C2=O)ccc1OC] | 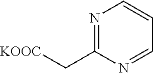 |
| 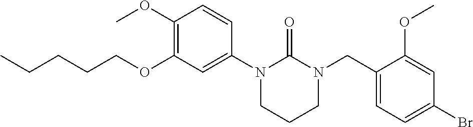 [SMILES: CCCCCOc1cc(N2CCCN(Cc3ccc(Br)cc3OC)C2=O)ccc1OC] | 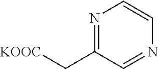 |
| 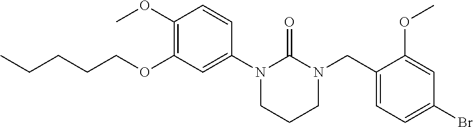 [SMILES: CCCCCOc1cc(N2CCCN(Cc3ccc(Br)cc3OC)C2=O)ccc1OC] | 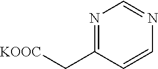 |
| 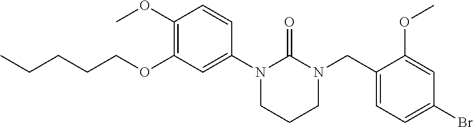 [SMILES: CCCCCOc1cc(N2CCCN(Cc3ccc(Br)cc3OC)C2=O)ccc1OC] | 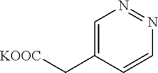 |
| 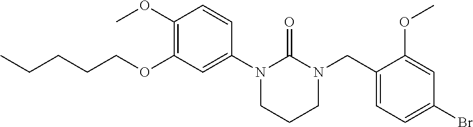 [SMILES: CCCCCOc1cc(N2CCCN(Cc3ccc(Br)cc3OC)C2=O)ccc1OC] | 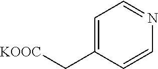 |

Compound

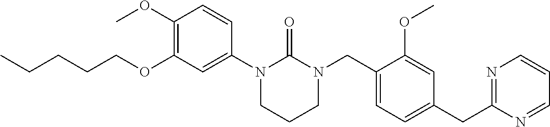 [SMILES: CCCCCOc1cc(N2CCCN(Cc3ccc(Cc4ncccn4)cc3OC)C2=O)ccc1OC]

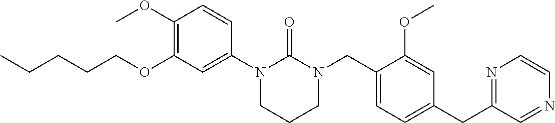 [SMILES: CCCCCOc1cc(N2CCCN(Cc3ccc(Cc4cnccn4)cc3OC)C2=O)ccc1OC]

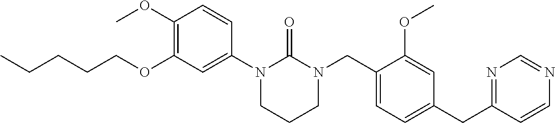 [SMILES: CCCCCOc1cc(N2CCCN(Cc3ccc(Cc4ccncn4)cc3OC)C2=O)ccc1OC]

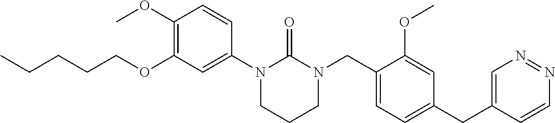 [SMILES: CCCCCOc1cc(N2CCCN(Cc3ccc(Cc4ccnnc4)cc3OC)C2=O)ccc1OC]

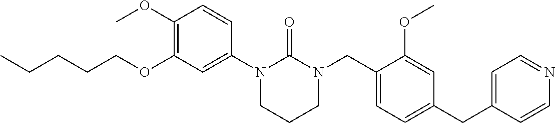 [SMILES: CCCCCOc1cc(N2CCCN(Cc3ccc(Cc4ccncc4)cc3OC)C2=O)ccc1OC]

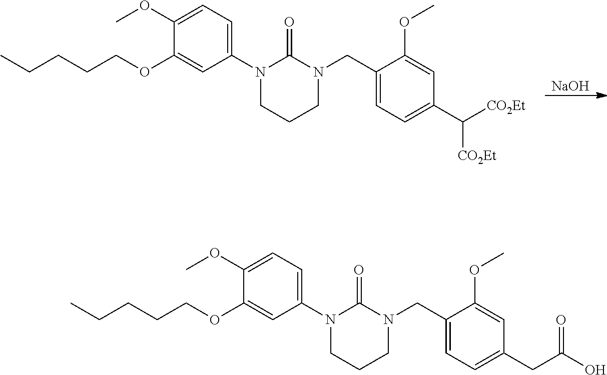 [SMILES: CCCCCOc1cc(N2CCCN(Cc3ccc(C(C(=O)OCC)C(=O)OCC)cc3OC)C2=O)ccc1OC ; "NaOH" ; CCCCCOc1cc(N2CCCN(Cc3ccc(CC(=O)O)cc3OC)C2=O)ccc1OC]

A solution of diethyl 2-(3-methoxy-4-((3-(4-methoxy-3-pentyloxy)phenyl-2-oxotetrahydropyrimidin-1(2H)-yl)methyl)phenyl)malonate (14.0 g, 24 mmol) and sodium hydroxide (2.0 g, 50 mmol) in a 1:2 v/v mixture of ethanol and water (300 mL) is refluxed for 12 hours. After cooling to room temperature, the mixture is washed with 1:1 v/v ethyl acetate/hexane mixture and, these washes are discarded. The remaining aqueous layer is acidified to pH<2 with 1N hydrochloric acid then refluxed for 2 h. After cooling to room temperature, the mixture is extracted with ethyl acetate. The combined organic phases are washed with brine, then dried over anhydrous sodium sulfate, filtered, and concentrated in vacuo to afford 2-(3-methoxy-4-((3-(4-methoxy-3-(pentyloxy)phenyl)-2-oxotetrahydropyrimidin-1(2H)-yl)methyl)phenyl)acetic acid.

The following compounds are prepared in a similar manner as described above.

| Di-ester | Compound |
|---|---|
| 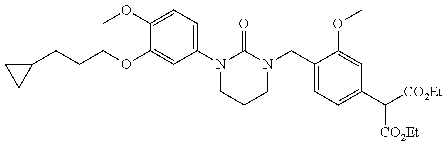 [SMILES: CCOC(=O)C(C(=O)OCC)c1ccc(CN2CCCN(c3ccc(OC)c(OCCCC4CC4)c3)C2=O)c(OC)c1] | 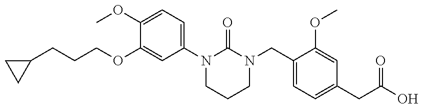 [SMILES: COc1cc(CC(=O)O)ccc1CN1CCCN(c2ccc(OC)c(OCCCC3CC3)c2)C1=O] |
| 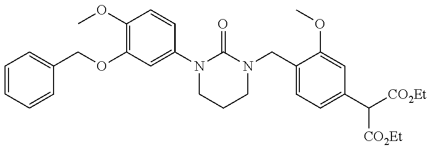 [SMILES: CCOC(=O)C(C(=O)OCC)c1ccc(CN2CCCN(c3ccc(OC)c(OCc4ccccc4)c3)C2=O)c(OC)c1] | 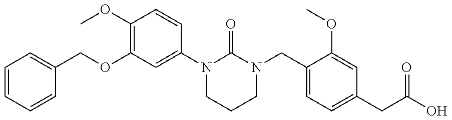 [SMILES: COc1cc(CC(=O)O)ccc1CN1CCCN(c2ccc(OC)c(OCc3ccccc3)c2)C1=O] |
| 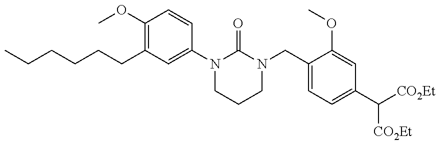 [SMILES: CCCCCCc1cc(N2CCCN(Cc3ccc(C(C(=O)OCC)C(=O)OCC)cc3OC)C2=O)ccc1OC] | 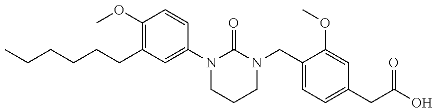 [SMILES: CCCCCCc1cc(N2CCCN(Cc3ccc(CC(=O)O)cc3OC)C2=O)ccc1OC] |
| 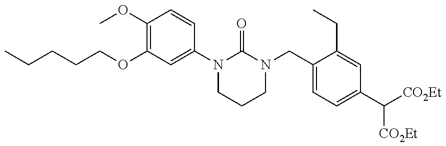 [SMILES: CCCCCOc1cc(N2CCCN(Cc3ccc(C(C(=O)OCC)C(=O)OCC)cc3CC)C2=O)ccc1OC] | 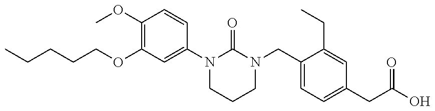 [SMILES: CCCCCOc1cc(N2CCCN(Cc3ccc(CC(=O)O)cc3CC)C2=O)ccc1OC] |

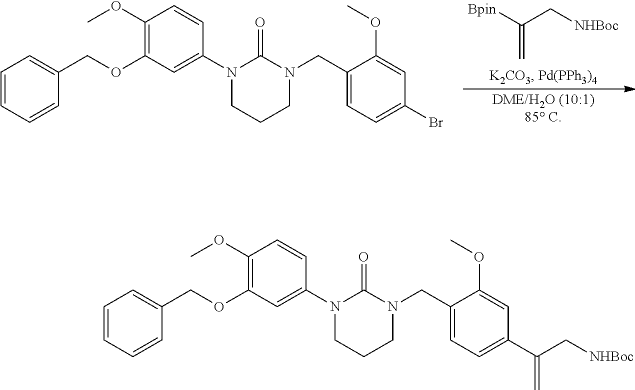

A round bottom flask is charged with 1-(3-(benzyloxy)-4-methoxyphenyl)-3-(4-bromo-2-methoxybenzyl)tetrahydropyrimidin-2(1H)-one (1.15 g, 2.25 mmol), potassium carbonate (970 mg, 7.02 mmol), and tert-butyl (2-(4,4,5,5-tetramethyl-1,3,2-dioxaborolan-2-yl)allyl)carbamate (1.08 g, 3.81 mmol). The flask is evacuated and purged with nitrogen three times. Anhydrous 1,2-dimethoxyethane (18 mL) and water (1.8 mL) are added, followed tetrakis(triphenylphosphine)palladium(0) (300 mg, 0.026 mmol). The reaction is heated to 85° C. and stirred at this temperature overnight. After cooling to room temperature, the reaction is diluted with methanol and filtered through CELITE®. The filter cake is thoroughly washed with methanol. The filtered solution is concentrated in vacuo. Purification by column chromatography over silica gel afforded tert-butyl (2-(4-((3-(3-(benzyloxy)-4-methoxyphenyl)-2-oxotetrahydropyrimidin-1(2H)-yl)methyl)-3-methoxyphenyl)allyl)carbamate.

The following compounds are prepared in a similar manner as described above:

| Halide | Boronate |
|---|---|
| 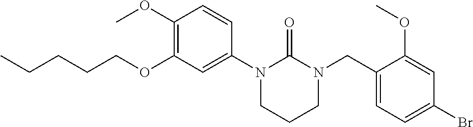 [SMILES: CCCCCOc1cc(N2CCCN(Cc3ccc(Br)cc3OC)C2=O)ccc1OC] | 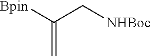 |

Compound

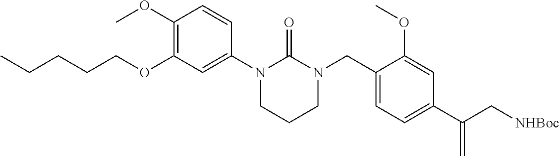 [SMILES: C=C(CNC(=O)OC(C)(C)C)c1ccc(CN2CCCN(c3ccc(OC)c(OCCCCC)c3)C2=O)c(OC)c1]

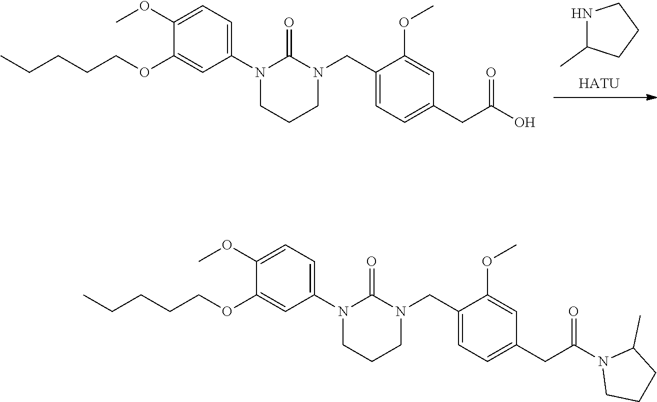

2-(3-methoxy-4-((3-(4-methoxy-3-(pentyloxy)phenyl)-2-oxotetrahydropyrimidin-1(2H)-yl)methyl)phenyl)acetic acid (40 mg, 0.09 mmol) is dissolved in N,N-dimethylformamide (1 mL), then N-[(dimethylamino)-1H-1,2,3-triazolo-[4,5-b]pyridin-1-ylmethylene]-N-methylmethanaminium hexafluorophosphate N-oxide (HATU, 38 mg, 0.10 mmol), 2-methylpyrrolidine (10 uL, 0.09 mmol), and triethylamine (18 uL, 0.13 mmol) are added. The reaction mixture is stirred at room temperature for 2 hours. Water is added, and the reaction mixture is extracted with ethyl acetate. The organic layers are dried over anhydrous sodium sulfate, filtered, and concentrated in vacuo. Purification by column chromatography over silica afforded 1-(4-methoxy-3-(pentyloxy)phenyl)-3-(2-methoxy-4-(2-(2-methylpyrrolidin-1-yl)-2-oxoethyl)benzyl)tetrahydropyrimidin-2(1H)-one.

The following compounds are prepared in a similar manner as described above.

| Acid | Amine | Compound |
|---|---|---|
| 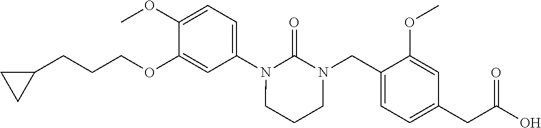 [SMILES: COc1cc(CC(=O)O)ccc1CN1CCCN(c2ccc(OC)c(OCCCC3CC3)c2)C1=O] | 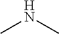 | 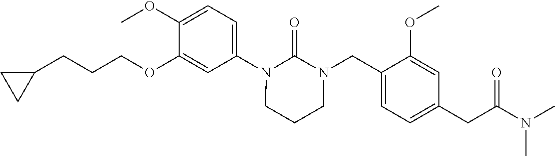 [SMILES: COc1cc(CC(=O)N(C)C)ccc1CN1CCCN(c2ccc(OC)c(OCCCC3CC3)c2)C1=O] |
| 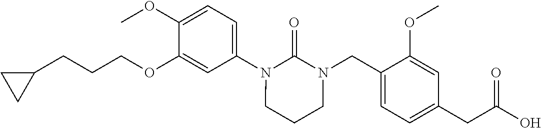 [SMILES: COc1cc(CC(=O)O)ccc1CN1CCCN(c2ccc(OC)c(OCCCC3CC3)c2)C1=O] | 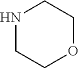 | 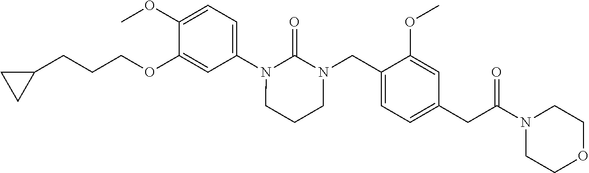 [SMILES: COc1cc(CC(=O)N2CCOCC2)ccc1CN1CCCN(c2ccc(OC)c(OCCCC3CC3)c2)C1=O] |
| 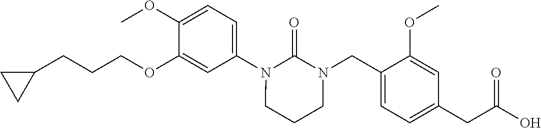 [SMILES: COc1cc(CC(=O)O)ccc1CN1CCCN(c2ccc(OC)c(OCCCC3CC3)c2)C1=O] | 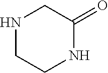 | 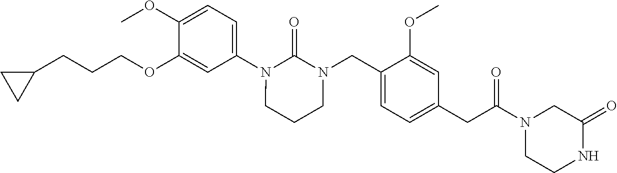 [SMILES: COc1cc(CC(=O)N2CCNC(=O)C2)ccc1CN1CCCN(c2ccc(OC)c(OCCCC3CC3)c2)C1=O] |
| 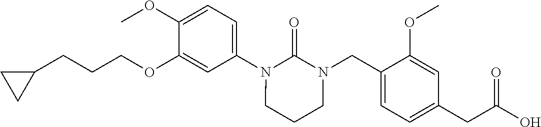 [SMILES: COc1cc(CC(=O)O)ccc1CN1CCCN(c2ccc(OC)c(OCCCC3CC3)c2)C1=O] | 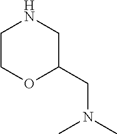 | 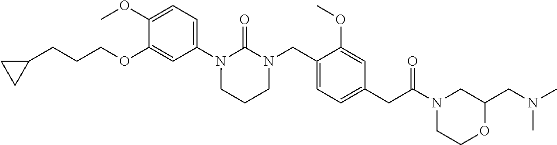 [SMILES: COc1cc(CC(=O)N2CCOC(CN(C)C)C2)ccc1CN1CCCN(c2ccc(OC)c(OCCCC3CC3)c2)C1=O] |
| 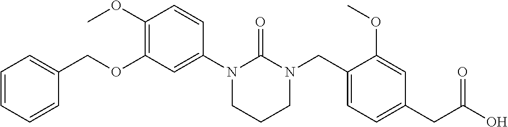 [SMILES: COc1cc(CC(=O)O)ccc1CN1CCCN(c2ccc(OC)c(OCc3ccccc3)c2)C1=O] | 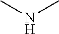 | 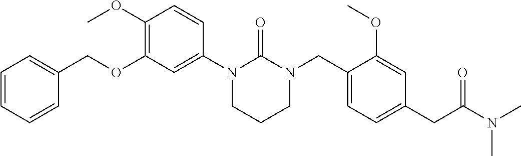 [SMILES: COc1cc(CC(=O)N(C)C)ccc1CN1CCCN(c2ccc(OC)c(OCc3ccccc3)c2)C1=O] |
| 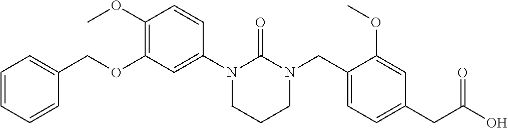 [SMILES: COc1cc(CC(=O)O)ccc1CN1CCCN(c2ccc(OC)c(OCc3ccccc3)c2)C1=O] | 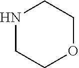 | 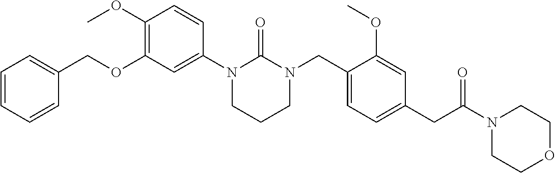 [SMILES: COc1cc(CC(=O)N2CCOCC2)ccc1CN1CCCN(c2ccc(OC)c(OCc3ccccc3)c2)C1=O] |
| 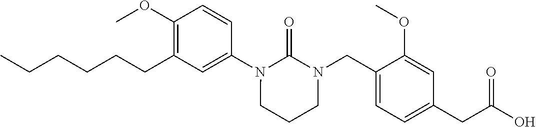 [SMILES: CCCCCCc1cc(N2CCCN(Cc3ccc(CC(=O)O)cc3OC)C2=O)ccc1OC] | 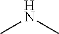 | 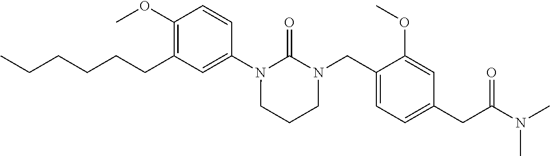 [SMILES: CCCCCCc1cc(N2CCCN(Cc3ccc(CC(=O)N(C)C)cc3OC)C2=O)ccc1OC] |
| 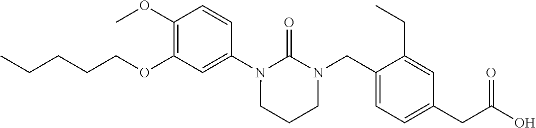 [SMILES: CCCCCOc1cc(N2CCCN(Cc3ccc(CC(=O)O)cc3CC)C2=O)ccc1OC] | 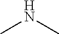 | 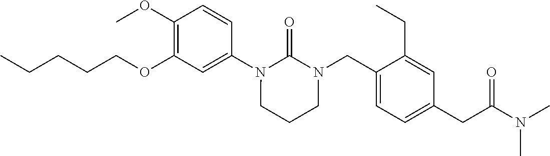 [SMILES: CCCCCOc1cc(N2CCCN(Cc3ccc(CC(=O)N(C)C)cc3CC)C2=O)ccc1OC] |

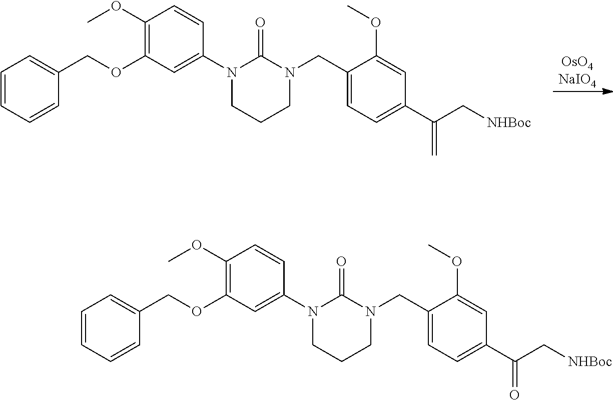 [SMILES: C=C(CNC(=O)OC(C)(C)C)c1ccc(CN2CCCN(c3ccc(OC)c(OCc4ccccc4)c3)C2=O)c(OC)c1 ; "OsO4NaIO4" ; COc1cc(C(=O)CNC(=O)OC(C)(C)C)ccc1CN1CCCN(c2ccc(OC)c(OCc3ccccc3)c2)C1=O]

To a solution of tert-butyl (2-(4-((3-(3-(benzyloxy)-4-methoxyphenyl)-2-oxotetrahydropyrimidin-1(2H)-yl)methyl)-3-methoxy phenyl)allyl)carbamate (1.00 g, 1.50 mmol) in anhydrous 1,4-dioxane (18.6 mL) and water (6.0 mL) is added 2,6-lutidine (373 uL, 3.17 mmol), sodium periodate (1.32 g, 6.17 mmol), and osmium tetroxide (209 uL, 0.033 mmol). The reaction is stirred at room temperature for 72 hours. The mixture is diluted with saturated aqueous sodium sulfite and extracted with dichloromethane. The combined organic extracts are dried over anhydrous magnesium sulfate, filtered, and concentrated in vacuo. Purification by column chromatography over silica gel afforded tert-butyl (2-(4-((3-(3-(benzyloxy)-4-methoxyphenyl)-2-oxotetrahydropyrimidin-1(2H)-yl)methyl)-3-methoxyphenyl)-2-oxoethyl)carbamate.

The following compounds are prepared in a similar manner as described above:

| Alkene | Compound |
|---|---|
| 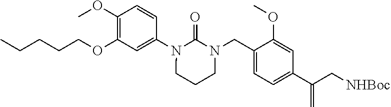 [SMILES: C=C(CNC(=O)OC(C)(C)C)c1ccc(CN2CCCN(c3ccc(OC)c(OCCCCC)c3)C2=O)c(OC)c1] | 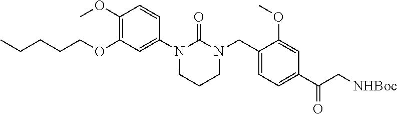 [SMILES: CCCCCOc1cc(N2CCCN(Cc3ccc(C(=O)CNC(=O)OC(C)(C)C)cc3OC)C2=O)ccc1OC] |

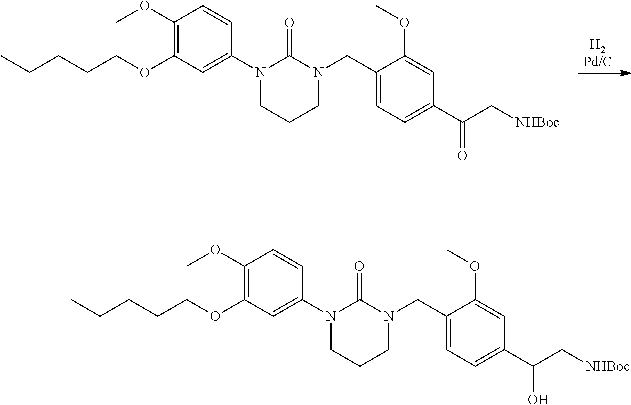 [SMILES: CCCCCOc1cc(N2CCCN(Cc3ccc(C(=O)CNC(=O)OC(C)(C)C)cc3OC)C2=O)ccc1OC ; "H2Pd/C" ; CCCCCOc1cc(N2CCCN(Cc3ccc(C(O)CNC(=O)OC(C)(C)C)cc3OC)C2=O)ccc1OC]

tert-Butyl (2-(3-methoxy-4-((3-(4-methoxy-3-(pentyloxy)phenyl)-2-oxotetrahydropyrimidin-1(2H)-yl)methyl)phenyl)-2-oxoethyl)carbamate (580 mg, 1.02 mmol) is dissolved in methanol (10 mL). 10 wt % palladium on carbon (400 mg, 0.38 mmol) is added. The reaction is stirred overnight at room temperature under a hydrogen atmosphere. The reaction is filtered through CELITE®. The filter cake is thoroughly washed with methanol. The filtered solution is concentrated in vacuo to afford tert-butyl (2-hydroxy-2-(3-methoxy-4-((3-(4-methoxy-3-(pentyloxy)phenyl)-2-oxotetrahydropyrimidin-1(2H)-yl)methyl)phenyl)ethyl)carbamate.

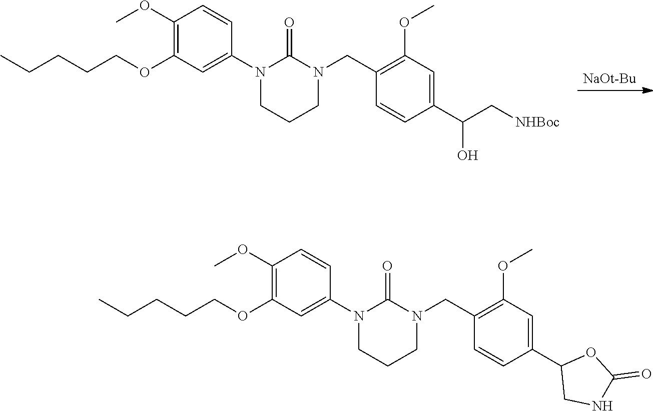 [SMILES: CCCCCOc1cc(N2CCCN(Cc3ccc(C(O)CNC(=O)OC(C)(C)C)cc3OC)C2=O)ccc1OC ; CCCCCOc1cc(N2CCCN(Cc3ccc(C4CNC(=O)O4)cc3OC)C2=O)ccc1OC ; "NaOt-Bu"]

To a mixture of tert-butyl (2-hydroxy-2-(3-methoxy-4-((3-(4-methoxy-3-(pentyloxy)phenyl)-2-oxotetrahydropyrimidin-1(2H)-yl)methyl)phenyl)ethyl)carbamate (100 mg, 0.18 mmol) in anhydrous N,N-dimethylformamide (2 mL) is added potassium tert-butoxide (65 mg, 0.57 mmol). The reaction is stirred at room temperature for 3 hours. The reaction mixture is concentrated in vacuo. Purification by column chromatography over reversed-phase C18 silica gel afforded 5-(3-methoxy-4-((3-(4-methoxy-3-(pentyloxy)phenyl)-2-oxotetrahydropyrimidin-1(2H)-yl)methyl)phenyl)oxazolidin-2-one.

The following compounds are prepared in a similar manner as described above:

| Protected amino alcohol | Compound |
|---|---|
| 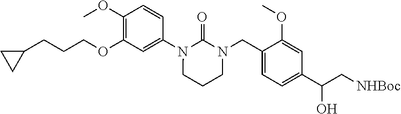 [SMILES: COc1cc(C(O)CNC(=O)OC(C)(C)C)ccc1CN1CCCN(c2ccc(OC)c(OCCCC3CC3)c2)C1=O] | 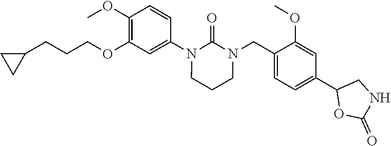 [SMILES: COc1cc(C2CNC(=O)O2)ccc1CN1CCCN(c2ccc(OC)c(OCCCC3CC3)c2)C1=O] |

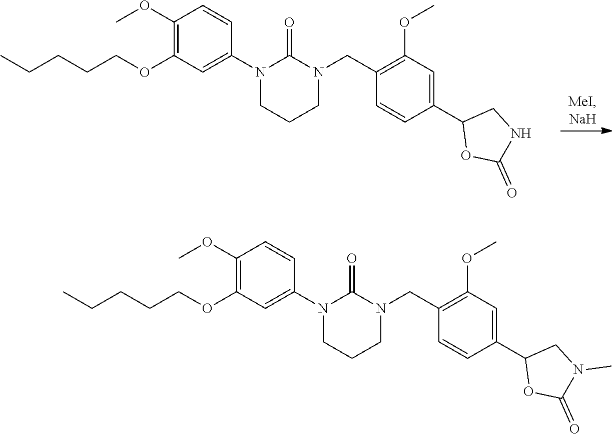

To a solution of 5-(3-methoxy-4-((3-(4-methoxy-3-(pentyloxy)phenyl)-2-oxotetrahydropyrimidin-1(2H)-yl)methyl)phenyl)oxazolidin-2-one (50 mg, 0.060 mmol) in anhydrous N,N-dimethylformamide (1 mL) is added sodium tert-butoxide (10 mg, 0.101 mmol) at 0° C. This solution is stirred at 0° C. for 15 minutes, before adding iodomethane (4 uL, 0.065 mmol). The reaction is stirred for 3 hours while gradually warming to room temperature. 1M aqueous sodium hydroxide is added, then the reaction is extracted with dichloromethane. The combined organic extracts are dried over anhydrous magnesium sulfate, filtered, and concentrated in vacuo. Purification by column chromatography over reversed-phase C18 silica gel afforded 5-(3-methoxy-4-((3-(4-methoxy-3-(pentyloxy)phenyl)-2-oxotetrahydropyrimidin-1(2H)-yl)methyl)phenyl)-3-methyloxazolidin-2-one.

The following compounds are prepared in a similar manner as described above:

| Amide | Halide | Compound |
|---|---|---|
| 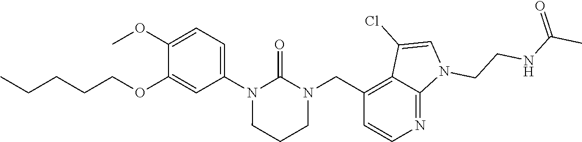 [SMILES: CCCCCOc1cc(N2CCCN(Cc3ccnc4c3c(Cl)cn4CCNC(C)=O)C2=O)ccc1OC] | 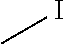 | 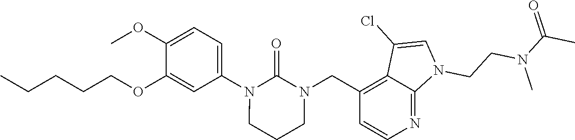 [SMILES: CCCCCOc1cc(N2CCCN(Cc3ccnc4c3c(Cl)cn4CCN(C)C(C)=O)C2=O)ccc1OC] |
| 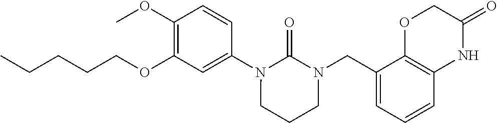 [SMILES: CCCCCOc1cc(N2CCCN(Cc3cccc4c3OCC(=O)N4)C2=O)ccc1OC] | 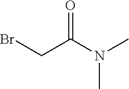 | 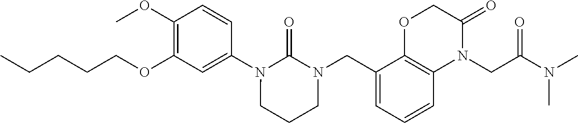 [SMILES: CCCCCOc1cc(N2CCCN(Cc3cccc4c3OCC(=O)N4CC(=O)N(C)C)C2=O)ccc1OC] |
| 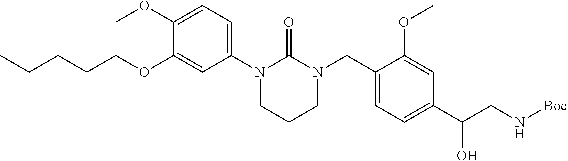 [SMILES: CCCCCOc1cc(N2CCCN(Cc3ccc(C(O)CNC(=O)OC(C)(C)C)cc3OC)C2=O)ccc1OC] | 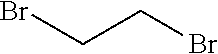 | 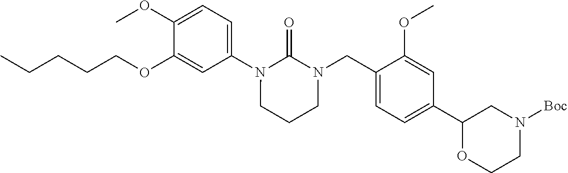 [SMILES: CCCCCOc1cc(N2CCCN(Cc3ccc(C4CN(C(=O)OC(C)(C)C)CCO4)cc3OC)C2=O)ccc1OC] |

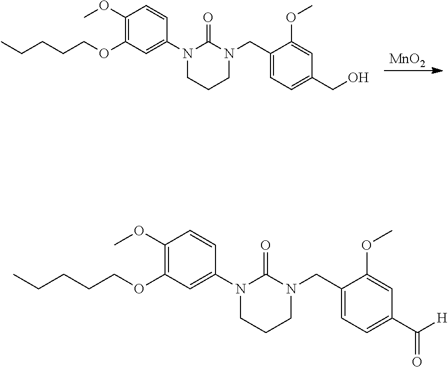

A solution of 1-(4-(hydroxymethyl)-2-methoxybenzyl)-3-(4-methoxy-3-(pentyloxy)phenyl)tetrahydropyrimidin-2(1H)-one (150 mg, 0.34 mmol) in anhydrous dichloromethane (3.4 mL) is treated with activated manganese (IV) oxide (589 mg, 6.77 mmol). The resulting suspension is stirred overnight at room temperature. The reaction is filtered through CELITE®, and the filter cake is thoroughly washed with dichloromethane. The filtered solution is concentrated in vacuo. Purification by column chromatography over silica gel afforded 3-methoxy-4-((3-(4-methoxy-3-(pentyloxy)phenyl)-2-oxotetrahydropyrimidin-1(2H)-yl)methyl)benzaldehyde.

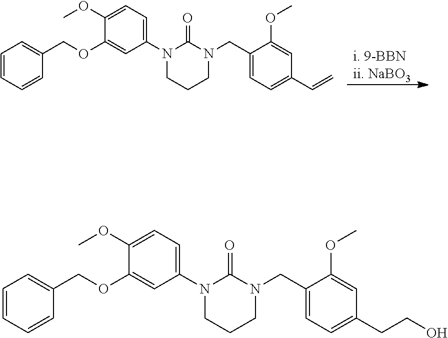

To a solution of 1-(3-(benzyloxy)-4-methoxyphenyl)-3-(2-methoxy-4-vinylbenzyl)tetrahydropyrimidin-2(1H)-one (601 mg, 1.22 mmol) in anhydrous tetrahydrofuran (3.7 mL) is added 9-borabicyclo[3.3.1]nonane (9-BBN, 0.5M in tetrahydrofuran, 10 mL, 5.0 mmol) slowly. The reaction is stirred at room temperature for 3 hours. A suspension of sodium perborate tetrahydrate (1.44 g, 9.11 mmol) in water (20 mL) is added. The resulting mixture is stirred 16 hours at room temperature. Water is added, then the reaction is extracted with dichloromethane. The combined organic extracts are dried over magnesium sulfate, filtered, and concentrated in vacuo. Purification by column chromatography over silica gel afforded 1-(3-(benzyloxy)-4-methoxyphenyl)-3-(4-(2-hydroxyethyl)-2-methoxybenzyl)tetrahydropyrimidin-2(1H)-one.

The following compounds are prepared in a similar manner as described above.

| Alkene | Compound |
|---|---|
| 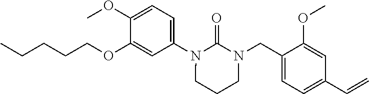 [SMILES: C=Cc1ccc(CN2CCCN(c3ccc(OC)c(OCCCCC)c3)C2=O)c(OC)c1] | 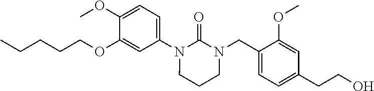 [SMILES: CCCCCOc1cc(N2CCCN(Cc3ccc(CCO)cc3OC)C2=O)ccc1OC] |
| 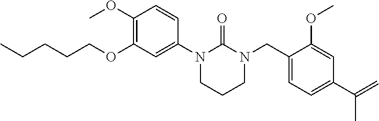 [SMILES: C=C(C)c1ccc(CN2CCCN(c3ccc(OC)c(OCCCCC)c3)C2=O)c(OC)c1] | 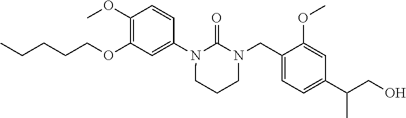 [SMILES: CCCCCOc1cc(N2CCCN(Cc3ccc(C(C)CO)cc3OC)C2=O)ccc1OC] |
| 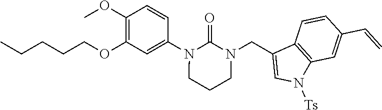 [SMILES: C=Cc1ccc2c(CN3CCCN(c4ccc(OC)c(OCCCCC)c4)C3=O)cn(S(=O)(=O)c3ccc(C)cc3)c2c1] | 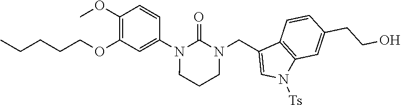 [SMILES: CCCCCOc1cc(N2CCCN(Cc3cn(S(=O)(=O)c4ccc(C)cc4)c4cc(CCO)ccc34)C2=O)ccc1OC] |

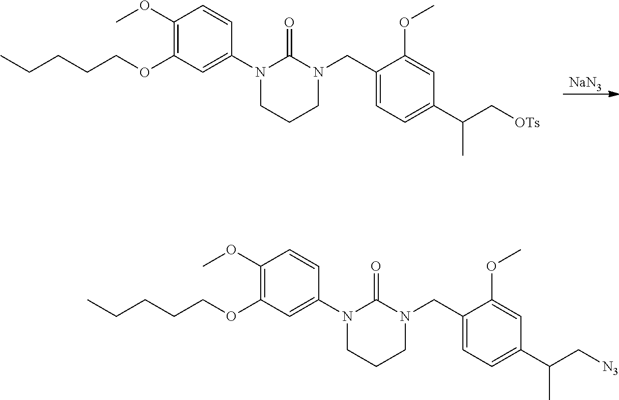 [SMILES: CCCCCOc1cc(N2CCCN(Cc3ccc(C(C)COS(=O)(=O)c4ccc(C)cc4)cc3OC)C2=O)ccc1OC ; CCCCCOc1cc(N2CCCN(Cc3ccc(C(C)CN=[N+]=[N-])cc3OC)C2=O)ccc1OC ; "NaN3"]

To a solution of 2-(3-methoxy-4-((3-(4-methoxy-3-(pentyloxy)phenyl)-2-oxotetrahydropyrimidin-1(2H)-yl)methyl)phenyl)propyl 4-methylbenzenesulfonate (492 mg, 0.78 mmol) in anhydrous N,N-dimethylformamide (5.6 mL) is added sodium azide (203 mg, 3.11 mmol). The reaction is heated to 55° C. and stirred 3 hours at this temperature. Water is added, then the reaction is extracted with dichloromethane. The combined organic layers are dried over anhydrous sodium sulfate, filtered, and concentrated in vacuo to afford 1-(4-(1-azidopropan-2-yl)-2-methoxybenzyl)-3-(4-methoxy-3-(pentyloxy)phenyl)tetrahydropyrimidin-2(1H)-one.

The following compounds are prepared in a similar manner as described above.

| Tosylate | Compound |
|---|---|
| 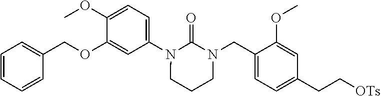 [SMILES: COc1cc(CCOS(=O)(=O)c2ccc(C)cc2)ccc1CN1CCCN(c2ccc(OC)c(OCc3ccccc3)c2)C1=O] | 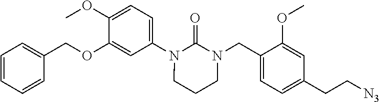 [SMILES: COc1cc(CCN=[N+]=[N-])ccc1CN1CCCN(c2ccc(OC)c(OCc3ccccc3)c2)C1=O] |

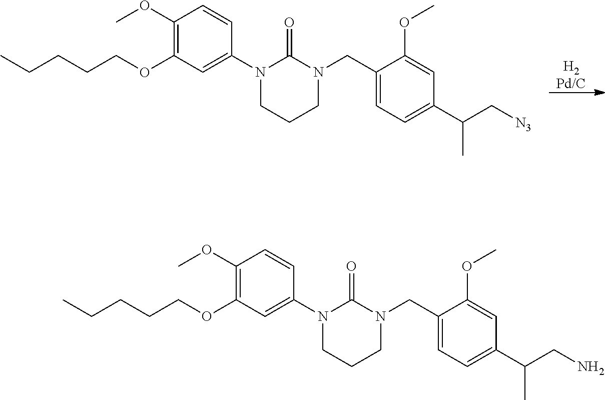 [SMILES: CCCCCOc1cc(N2CCCN(Cc3ccc(C(C)CN=[N+]=[N-])cc3OC)C2=O)ccc1OC ; "H2Pd/C" ; CCCCCOc1cc(N2CCCN(Cc3ccc(C(C)CN)cc3OC)C2=O)ccc1OC]

A solution of 1-(4-(1-azidopropan-2-yl)-2-methoxybenzyl)-3-(4-methoxy-3-(pentyloxy)phenyl)tetrahydropyrimidin-2(1H)-one (50 mg, 0.10 mmol) in ethyl acetate (1 mL) is treated with 10 wt % palladium on carbon (10 mg). The reaction vial is purged with hydrogen, then stirred at room temperature under a hydrogen atmosphere overnight. The reaction is filtered through CELITE® with ethyl acetate. The filter cake is thoroughly washed with ethyl acetate. The filtered solution is concentrated in vacuo, and purification by column chromatography over reversed-phase C18 silica gel afforded 1-(4-(1-aminopropan-2-yl)-2-methoxybenzyl)-3-(4-methoxy-3-(pentyloxy)phenyl)tetrahydropyrimidin-2(1H)-one.

The following compounds are prepared in a similar manner as described above.

| Azide | Compound |
|---|---|
| 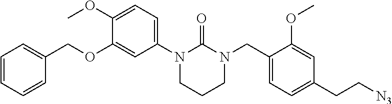 [SMILES: COc1cc(CCN=[N+]=[N-])ccc1CN1CCCN(c2ccc(OC)c(OCc3ccccc3)c2)C1=O] | 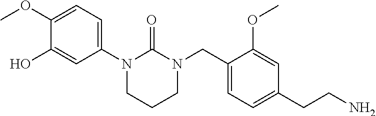 [SMILES: COc1ccc(N2CCCN(Cc3ccc(CCN)cc3OC)C2=O)cc1O] |

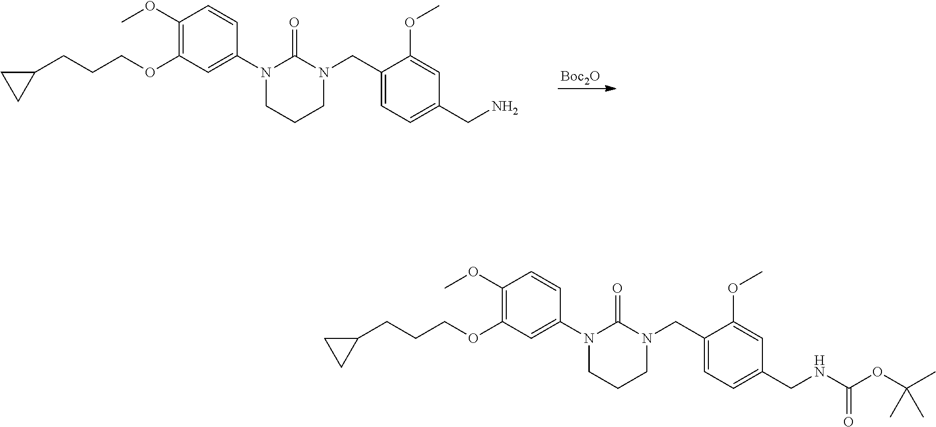 [SMILES: "Boc2O" ; COc1cc(CN)ccc1CN1CCCN(c2ccc(OC)c(OCCCC3CC3)c2)C1=O ; COc1cc(CNC(=O)OC(C)(C)C)ccc1CN1CCCN(c2ccc(OC)c(OCCCC3CC3)c2)C1=O]

A solution of 1-(4-(aminomethyl)-2-methoxybenzyl)-3-(3-(3-cyclopropylpropoxy)-4-methoxyphenyl)tetrahydropyrimidin-2(1H)-one (215 mg, 0.47 mmol) and 4-(N,N-dimethylamino)pyridine (6 mg, 0.05 mmol) in methanol (5 mL) is treated with di-tert butyl dicarbonate (123 mg, 0.56 mmol) and triethylamine (98 uL, 0.71 mmol). The reaction is stirred at room temperature overnight. The reaction is concentrated in vacuo. The residue material is taken up in dichloromethane and washed with saturated aqueous sodium bicarbonate solution, then saturated aqueous ammonium chloride solution, and water. The organic layers are then dried over anhydrous sodium sulfate, filtered, and concentrated in vacuo. Purification by column chromatography of silica gel afforded tert-butyl (4-((3-(3-(3-cyclopropylpropoxy)-4-methoxyphenyl)-2-oxotetrahydropyrimidin-1(2H)-yl)methyl)-3-methoxybenzyl)carbamate.

The following compounds are prepared in a similar manner as described above.

| Amine | Compound |
|---|---|
| 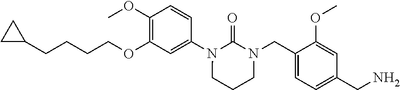 [SMILES: COc1cc(CN)ccc1CN1CCCN(c2ccc(OC)c(OCCCCC3CC3)c2)C1=O] | 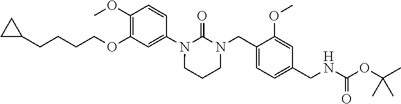 [SMILES: COc1cc(CNC(=O)OC(C)(C)C)ccc1CN1CCCN(c2ccc(OC)c(OCCCCC3CC3)c2)C1=O] |
| 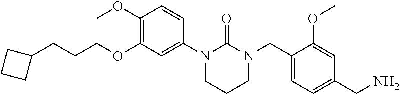 [SMILES: COc1cc(CN)ccc1CN1CCCN(c2ccc(OC)c(OCCCC3CCC3)c2)C1=O] | 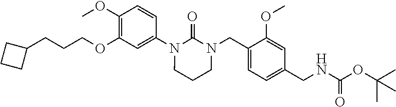 [SMILES: COc1cc(CNC(=O)OC(C)(C)C)ccc1CN1CCCN(c2ccc(OC)c(OCCCC3CCC3)c2)C1=O] |
| 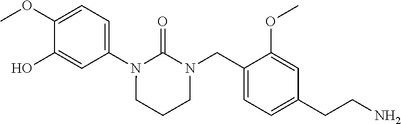 [SMILES: COc1ccc(N2CCCN(Cc3ccc(CCN)cc3OC)C2=O)cc1O] | 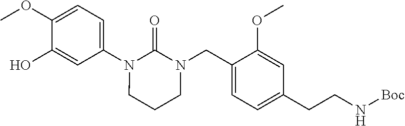 [SMILES: COc1ccc(N2CCCN(Cc3ccc(CCNC(=O)OC(C)(C)C)cc3OC)C2=O)cc1O] |
| 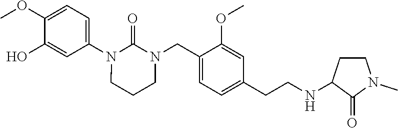 | 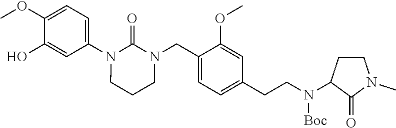 |

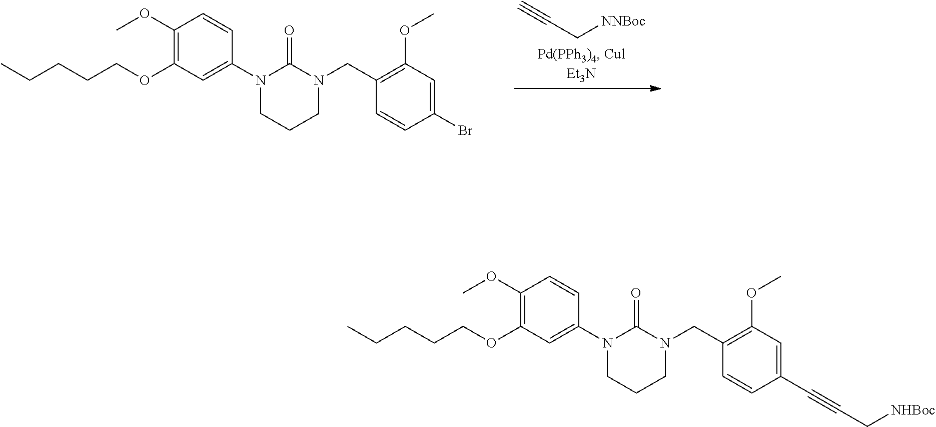

1-(4-bromo-2-methoxybenzyl)-3-(4-methoxy-3-(pentyloxy)phenyl)tetrahydropyrimidin-2(1H)-one (200 mg, 0.41 mmol), tetrakis(triphenylphosphine)palladium(0) (47 mg, 0.04 mmol), and copper(I) iodide (8 mg, 0.04 mmol) are placed in a 25 mL round bottom flask. The flask is evacuated and purged with nitrogen three times before adding anhydrous 1,4-dioxane (4 mL) and triethylamine (113 uL, 0.81 mmol). This mixture is sparged briefly with nitrogen before adding tert-butyl prop-2-yn-1-ylcarbamate (126 mg, 0.81 mmol). The reaction is stirred at 40° C. overnight under a nitrogen atmosphere. The reaction is cooled to room temperature then diluted with half-saturated aqueous ammonium chloride (10 mL). This mixture is extracted with ethyl acetate (4×10 mL). The combined organic layers are washed with brine (10 mL), dried over anhydrous magnesium sulfate, filtered, and concentrated in vacuo. Purification by column chromatography over silica gel afforded tert-butyl (3-(3-methoxy-4-((3-(4-methoxy-3-(pentyloxy)phenyl)-2-oxotetrahydropyrimidin-1(2H)-yl)methyl)phenyl)prop-2-yn-1-yl)carbamate.

The following compounds are prepared in a similar manner as described above.

| Aryl halide | Alkyne | Compound |
|---|---|---|
| 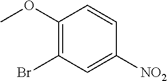 | 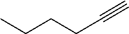 | 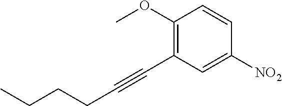 [SMILES: CCCCC#Cc1cc([N+](=O)[O-])ccc1OC] |
| 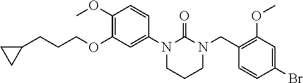 [SMILES: COc1cc(Br)ccc1CN1CCCN(c2ccc(OC)c(OCCCC3CC3)c2)C1=O] | 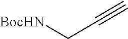 | 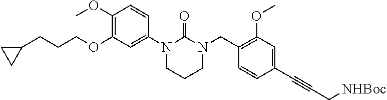 [SMILES: COc1cc(C#CCNC(=O)OC(C)(C)C)ccc1CN1CCCN(c2ccc(OC)c(OCCCC3CC3)c2)C1=O] |

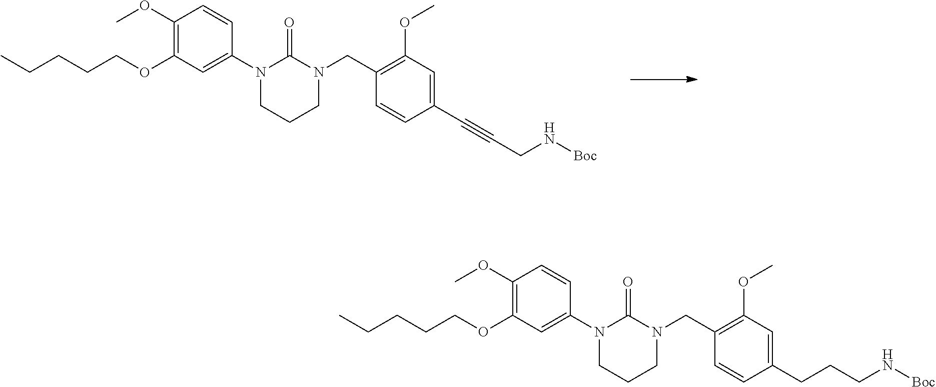 [SMILES: CCCCCOc1cc(N2CCCN(Cc3ccc(C#CCNC(=O)OC(C)(C)C)cc3OC)C2=O)ccc1OC ; CCCCCOc1cc(N2CCCN(Cc3ccc(CCCNC(=O)OC(C)(C)C)cc3OC)C2=O)ccc1OC]

tert-Butyl (3-(3-methoxy-4-((3-(4-methoxy-3-(pentyloxy)phenyl)-2-oxotetrahydropyrimidin-1(2H)-yl)methyl)phenyl)prop-2-yn-1-yl)carbamate (150 mg, 0.27 mmol) is dissolved in methanol (3 mL) and treated with 10 wt % palladium on carbon (50 mg). The reaction is stirred under a hydrogen atmosphere at room temperature for 5 hours. The solids are removed by filtration through CELITE® using additional methanol. The filter cake is thoroughly washed with methanol. The filtered solution is concentrated in vacuo to afford tert-butyl (3-(3-methoxy-4-((3-(4-methoxy-3-(pentyloxy)phenyl)-2-oxotetrahydropyrimidin-1(2H)-yl)methyl)phenyl)propyl)carbamate.

The following compounds are prepared in a similar manner as described above.

| Alkene or alkyne | Compound |
|---|---|
| 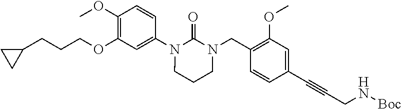 [SMILES: COc1cc(C#CCNC(=O)OC(C)(C)C)ccc1CN1CCCN(c2ccc(OC)c(OCCCC3CC3)c2)C1=O] | 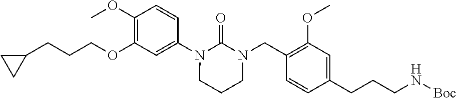 [SMILES: COc1cc(CCCNC(=O)OC(C)(C)C)ccc1CN1CCCN(c2ccc(OC)c(OCCCC3CC3)c2)C1=O] |
| 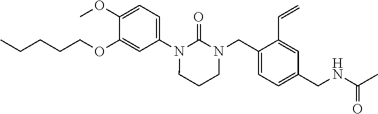 [SMILES: C=Cc1cc(CNC(C)=O)ccc1CN1CCCN(c2ccc(OC)c(OCCCCC)c2)C1=O] | 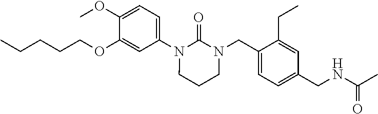 [SMILES: CCCCCOc1cc(N2CCCN(Cc3ccc(CNC(C)=O)cc3CC)C2=O)ccc1OC] |

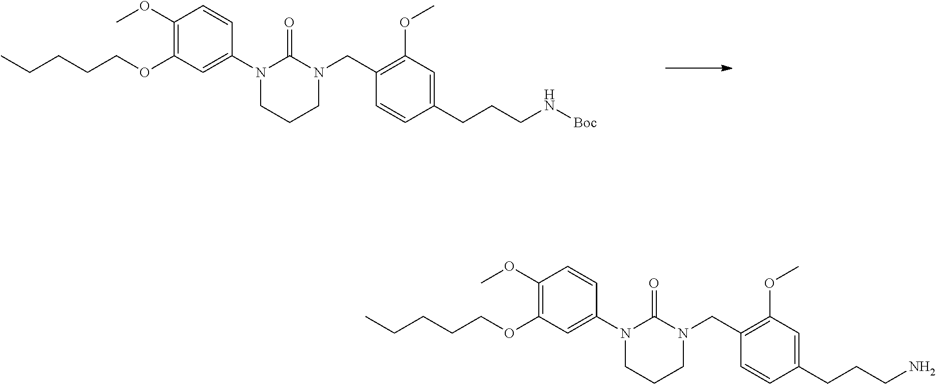 [SMILES: CCCCCOc1cc(N2CCCN(Cc3ccc(CCCNC(=O)OC(C)(C)C)cc3OC)C2=O)ccc1OC ; CCCCCOc1cc(N2CCCN(Cc3ccc(CCCN)cc3OC)C2=O)ccc1OC]

Neat trifluoroacetic acid (2 mL) is cooled to 0° C. before being treated with a solution of tert-butyl (3-(3-methoxy-4-((3-(4-methoxy-3-(pentyloxy)phenyl)-2-oxotetrahydropyrimidin-1(2H)-yl)methyl)phenyl)propyl)carbamate (140 mg, 0.25 mmol) in anhydrous dichloromethane (1 mL). The reaction is warmed to room temperature and stirred for 2 hours. Water is added, followed by saturated aqueous sodium bicarbonate solution until pH>8. The mixture is extracted with dichloromethane. The combined organic extracts are washed with saturated aqueous sodium bicarbonate solution, then dried over anhydrous sodium sulfate, filtered, and concentrated in vacuo. Purification by column chromatography over silica gel afforded 1-(4-(3-aminopropyl)-2-methoxybenzyl)-3-(4-methoxy-3-(pentyloxy)phenyl)tetrahydropyrimidin-2(1H)-one.

The following compounds are prepared in a similar manner as described above.

| Protected amine | Compound |
|---|---|
| 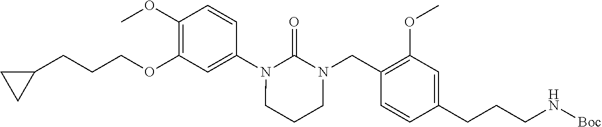 [SMILES: COc1cc(CCCNC(=O)OC(C)(C)C)ccc1CN1CCCN(c2ccc(OC)c(OCCCC3CC3)c2)C1=O] | 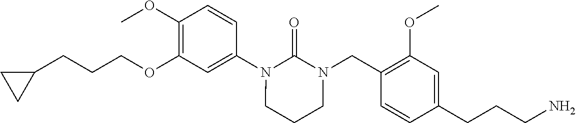 [SMILES: COc1cc(CCCN)ccc1CN1CCCN(c2ccc(OC)c(OCCCC3CC3)c2)C1=O] |
| 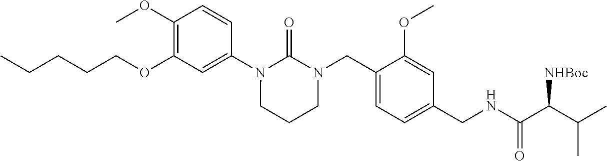 [SMILES: CCCCCOc1cc(N2CCCN(Cc3ccc(CNC(=O)[C@@H](NC(=O)OC(C)(C)C)C(C)C)cc3OC)C2=O)ccc1OC] | 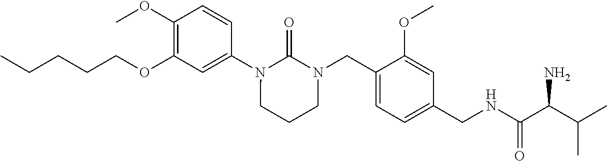 [SMILES: CCCCCOc1cc(N2CCCN(Cc3ccc(CNC(=O)[C@@H](N)C(C)C)cc3OC)C2=O)ccc1OC] |
| 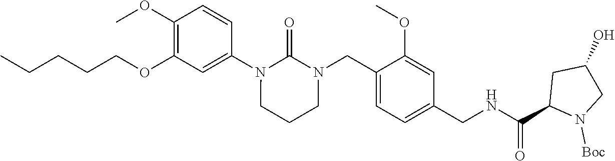 [SMILES: CCCCCOc1cc(N2CCCN(Cc3ccc(CNC(=O)[C@H]4C[C@H](O)CN4C(=O)OC(C)(C)C)cc3OC)C2=O)ccc1OC] | 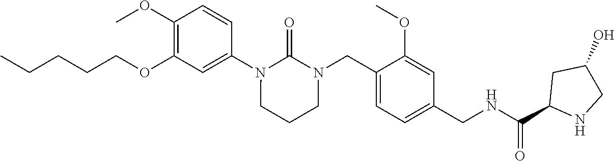 [SMILES: CCCCCOc1cc(N2CCCN(Cc3ccc(CNC(=O)[C@H]4C[C@H](O)CN4)cc3OC)C2=O)ccc1OC] |
| 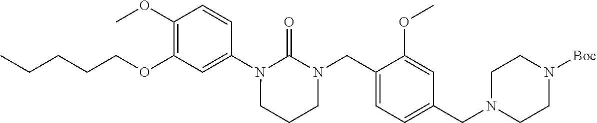 [SMILES: CCCCCOc1cc(N2CCCN(Cc3ccc(CN4CCN(C(=O)OC(C)(C)C)CC4)cc3OC)C2=O)ccc1OC] | 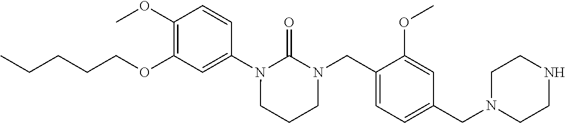 [SMILES: CCCCCOc1cc(N2CCCN(Cc3ccc(CN4CCNCC4)cc3OC)C2=O)ccc1OC] |
| 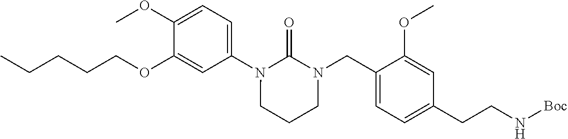 [SMILES: CCCCCOc1cc(N2CCCN(Cc3ccc(CCNC(=O)OC(C)(C)C)cc3OC)C2=O)ccc1OC] | 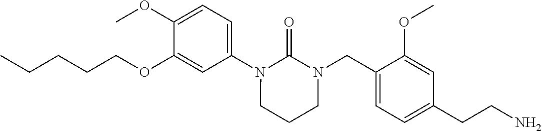 [SMILES: CCCCCOc1cc(N2CCCN(Cc3ccc(CCN)cc3OC)C2=O)ccc1OC] |
| 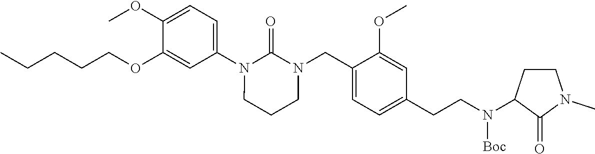 [SMILES: CCCCCOc1cc(N2CCCN(Cc3ccc(CCN(C(=O)OC(C)(C)C)C4CCN(C)C4=O)cc3OC)C2=O)ccc1OC] | 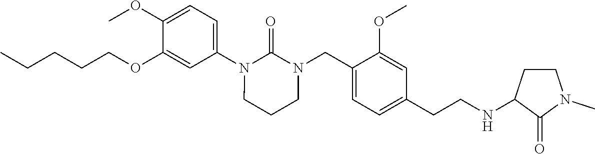 [SMILES: CCCCCOc1cc(N2CCCN(Cc3ccc(CCNC4CCN(C)C4=O)cc3OC)C2=O)ccc1OC] |
| 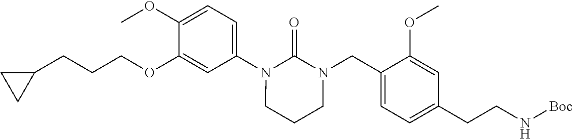 [SMILES: COc1cc(CCNC(=O)OC(C)(C)C)ccc1CN1CCCN(c2ccc(OC)c(OCCCC3CC3)c2)C1=O] | 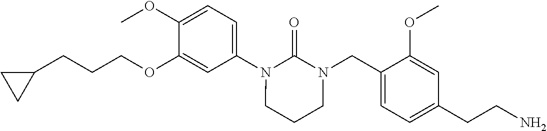 [SMILES: COc1cc(CCN)ccc1CN1CCCN(c2ccc(OC)c(OCCCC3CC3)c2)C1=O] |
| 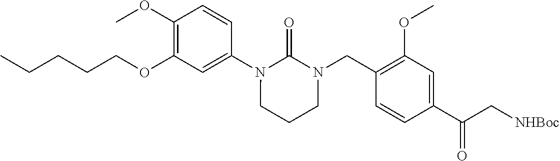 [SMILES: CCCCCOc1cc(N2CCCN(Cc3ccc(C(=O)CNC(=O)OC(C)(C)C)cc3OC)C2=O)ccc1OC] | 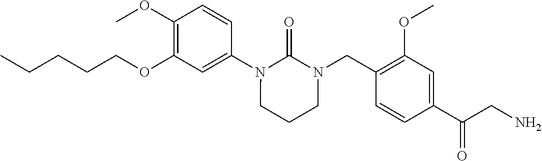 [SMILES: CCCCCOc1cc(N2CCCN(Cc3ccc(C(=O)CN)cc3OC)C2=O)ccc1OC] |
| 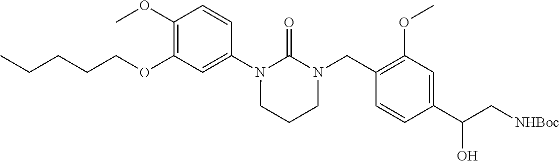 [SMILES: CCCCCOc1cc(N2CCCN(Cc3ccc(C(O)CNC(=O)OC(C)(C)C)cc3OC)C2=O)ccc1OC] | 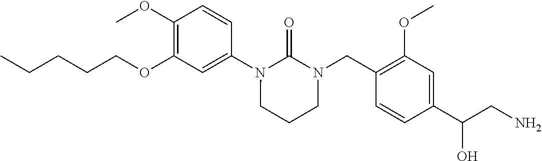 [SMILES: CCCCCOc1cc(N2CCCN(Cc3ccc(C(O)CN)cc3OC)C2=O)ccc1OC] |
| 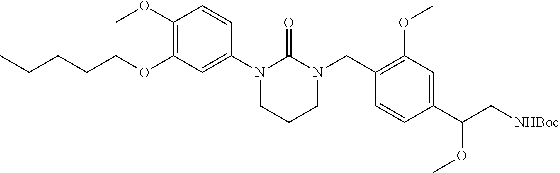 [SMILES: CCCCCOc1cc(N2CCCN(Cc3ccc(C(CNC(=O)OC(C)(C)C)OC)cc3OC)C2=O)ccc1OC] | 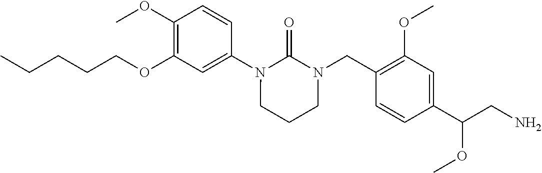 [SMILES: CCCCCOc1cc(N2CCCN(Cc3ccc(C(CN)OC)cc3OC)C2=O)ccc1OC] |
| 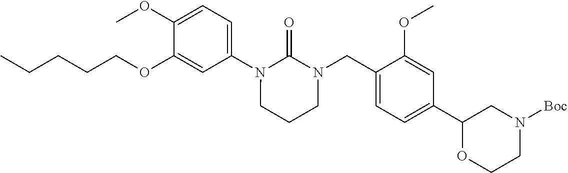 [SMILES: CCCCCOc1cc(N2CCCN(Cc3ccc(C4CN(C(=O)OC(C)(C)C)CCO4)cc3OC)C2=O)ccc1OC] | 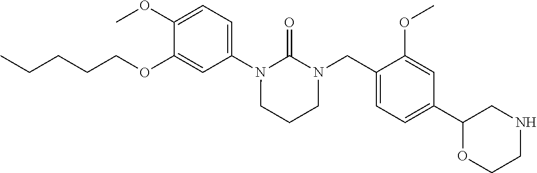 [SMILES: CCCCCOc1cc(N2CCCN(Cc3ccc(C4CNCCO4)cc3OC)C2=O)ccc1OC] |
| 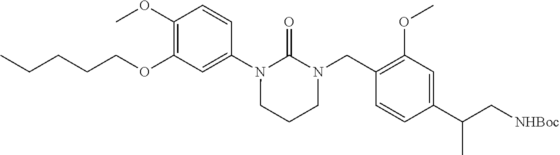 [SMILES: CCCCCOc1cc(N2CCCN(Cc3ccc(C(C)CNC(=O)OC(C)(C)C)cc3OC)C2=O)ccc1OC] | 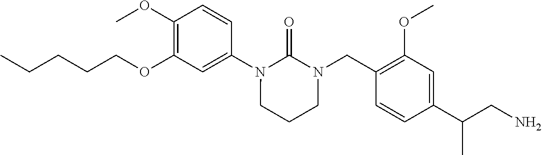 [SMILES: CCCCCOc1cc(N2CCCN(Cc3ccc(C(C)CN)cc3OC)C2=O)ccc1OC] |
| 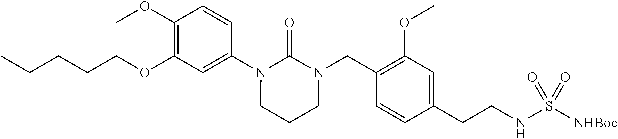 [SMILES: CCCCCOc1cc(N2CCCN(Cc3ccc(CCNS(=O)(=O)NC(=O)OC(C)(C)C)cc3OC)C2=O)ccc1OC] | 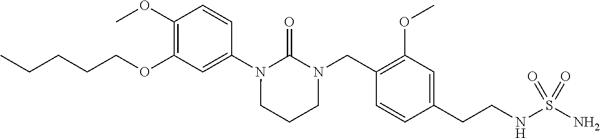 [SMILES: CCCCCOc1cc(N2CCCN(Cc3ccc(CCNS(N)(=O)=O)cc3OC)C2=O)ccc1OC] |
| 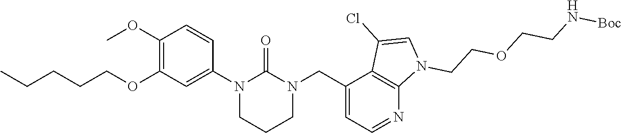 [SMILES: CCCCCOc1cc(N2CCCN(Cc3ccnc4c3c(Cl)cn4CCOCCNC(=O)OC(C)(C)C)C2=O)ccc1OC] | 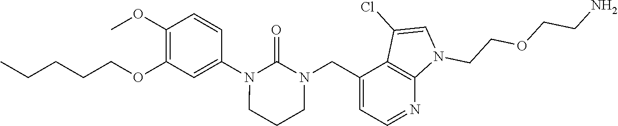 [SMILES: CCCCCOc1cc(N2CCCN(Cc3ccnc4c3c(Cl)cn4CCOCCN)C2=O)ccc1OC] |
| 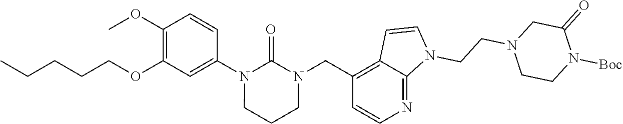 [SMILES: CCCCCOc1cc(N2CCCN(Cc3ccnc4c3ccn4CCN3CCN(C(=O)OC(C)(C)C)C(=O)C3)C2=O)ccc1OC] | 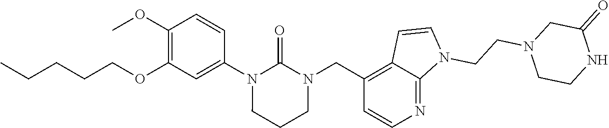 [SMILES: CCCCCOc1cc(N2CCCN(Cc3ccnc4c3ccn4CCN3CCNC(=O)C3)C2=O)ccc1OC] |
| 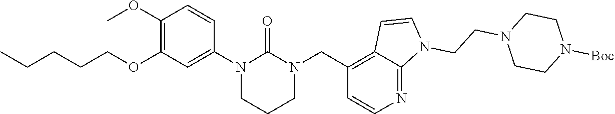 [SMILES: CCCCCOc1cc(N2CCCN(Cc3ccnc4c3ccn4CCN3CCN(C(=O)OC(C)(C)C)CC3)C2=O)ccc1OC] | 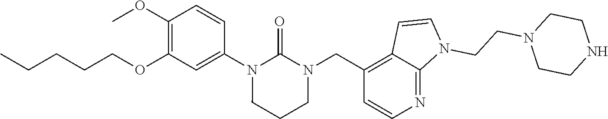 [SMILES: CCCCCOc1cc(N2CCCN(Cc3ccnc4c3ccn4CCN3CCNCC3)C2=O)ccc1OC] |
| 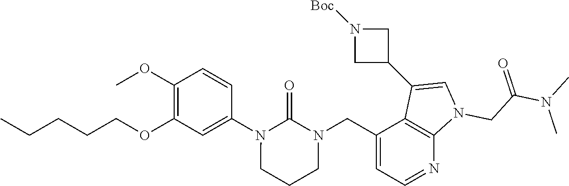 [SMILES: CCCCCOc1cc(N2CCCN(Cc3ccnc4c3c(C3CN(C(=O)OC(C)(C)C)C3)cn4CC(=O)N(C)C)C2=O)ccc1OC] | 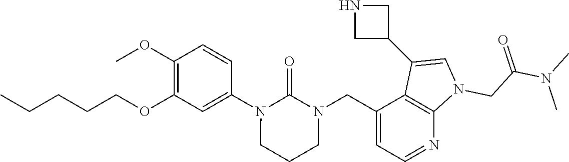 [SMILES: CCCCCOc1cc(N2CCCN(Cc3ccnc4c3c(C3CNC3)cn4CC(=O)N(C)C)C2=O)ccc1OC] |
| 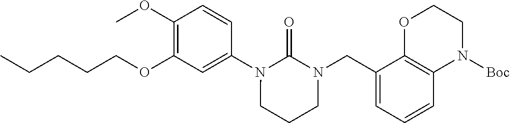 [SMILES: CCCCCOc1cc(N2CCCN(Cc3cccc4c3OCCN4C(=O)OC(C)(C)C)C2=O)ccc1OC] | 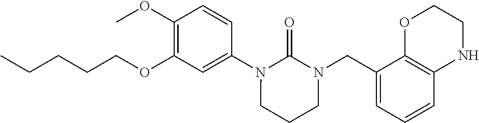 [SMILES: CCCCCOc1cc(N2CCCN(Cc3cccc4c3OCCN4)C2=O)ccc1OC] |

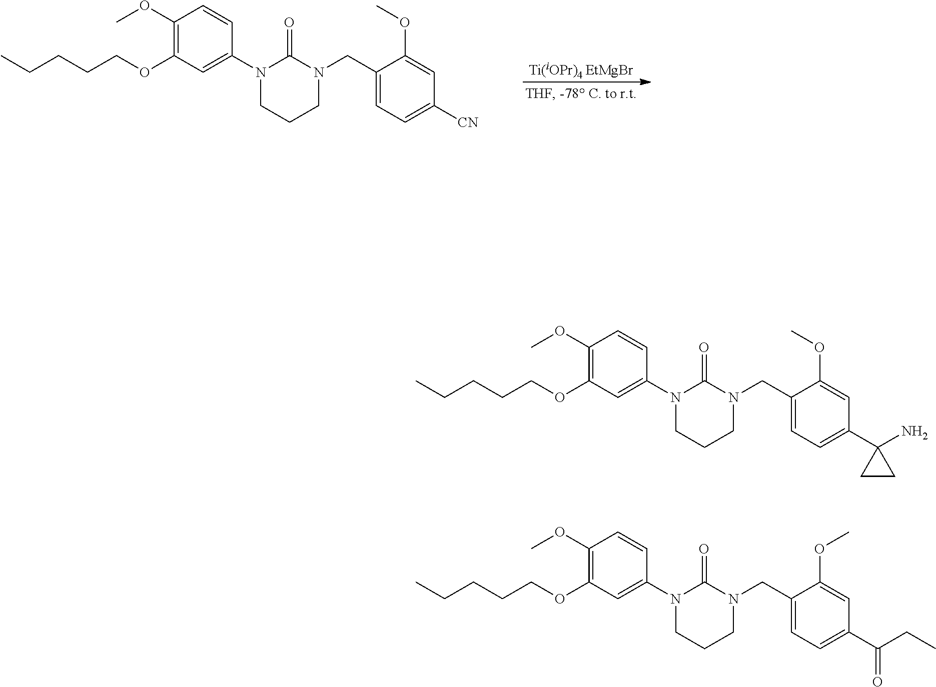 [SMILES: CCCCCOc1cc(N2CCCN(Cc3ccc(C#N)cc3OC)C2=O)ccc1OC ; CCCCCOc1cc(N2CCCN(Cc3ccc(C4(N)CC4)cc3OC)C2=O)ccc1OC ; CCCCCOc1cc(N2CCCN(Cc3ccc(C(=O)CC)cc3OC)C2=O)ccc1OC]

A solution of 3-methoxy-4-((3-(4-methoxy-3-(pentyloxy)phenyl)-2-oxotetrahydropyrimidin-1(2H)-yl)methyl)benzonitrile (500 mg, 1.14 mmol) in anhydrous tetrahydrofuran (5.7 mL) is cooled to −78° C. and treated with titanium(IV) isopropoxide (0.37 mL, 1.26 mmol). Ethylmagnesium bromide (3.0M in diethyl ether, 0.84 mL, 2.51 mmol) is added dropwise. The reaction is stirred for 10 minutes at −78° C., then warmed to room temperature and stirred another 2 hours. The reaction is diluted with 1N hydrochloric acid and diethyl ether. 10 wt % aqueous sodium hydroxide solution is added last. The layers are separated, and the aqueous layer is extracted with diethyl ether. The combined organic layers are dried with anhydrous sodium sulfate, filtered, and concentrated in vacuo. Purification by preparative HPLC afforded 1-(4-(1-aminocyclopropyl)-2-methoxybenzyl)-3-(4-methoxy-3-(pentyloxy)phenyl)tetrahydropyrimidin-2(1H)-one & 1-(4-methoxy-3-(pentyloxy)phenyl)-3-(2-methoxy-4-propionylbenzyl)tetrahydropyrimidin-2(1H)-one.

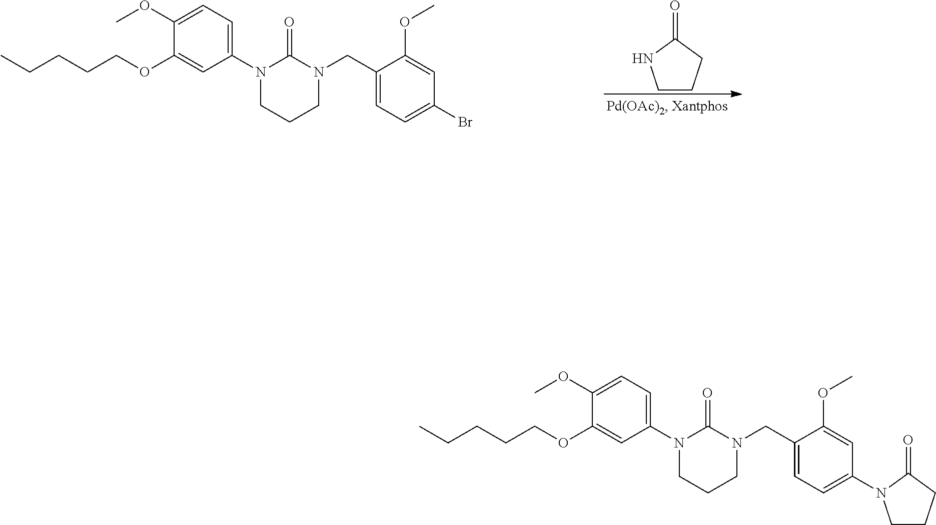 [SMILES: CCCCCOc1cc(N2CCCN(Cc3ccc(Br)cc3OC)C2=O)ccc1OC ; O=C1CCCN1 ; CCCCCOc1cc(N2CCCN(Cc3ccc(N4CCCC4=O)cc3OC)C2=O)ccc1OC]

A round bottom flask is charged with 1-(4-bromo-2-methoxybenzyl)-3-(4-methoxy-3-(pentyloxy)phenyl)tetrahydropyrimidin-2(1H)-one (60 mg, 0.12 mmol), palladium diacetate (2 mg, 0.01 mmol), 4,5-bis(diphenylphosphino)-9,9-dimethylxanthene (Xantphos, 9 mg, 0.02 mmol), and anhydrous cesium carbonate (46 mg, 0.14 mmol). The flask is evacuated and purged with nitrogen three times. Anhydrous 1,4-dioxane (1.2 mL) and 2-pyrrolidinone (14 uL, 0.18 mmol) are added. The reaction is heated to 100° C. and stirred overnight. The reaction is cooled to room temperature then diluted with water and extracted with dichloromethane. The combined organic layers are dried over anhydrous sodium sulfate, filtered, and concentrated in vacuo. Purification by column chromatography over silica gel afforded 1-(4-methoxy-3-(pentyloxy)phenyl)-3-(2-methoxy-4-(2-oxopyrrolidin-1-yl)benzyl)tetrahydropyrimidin-2(1H)-one.

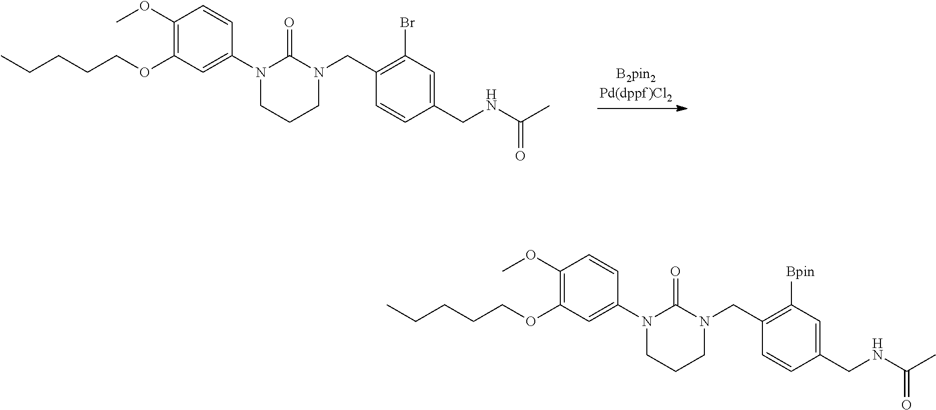

A round bottom flask is charged with N-(3-bromo-4-((3-(4-methoxy-3-(pentyloxy)phenyl)-2-oxotetrahydropyrimidin-1(2H)-yl)methyl)benzyl)acetamide (100 mg, 0.19 mmol), bis(pinacolato)diboron (57 mg, 0.23 mmol), [1,1′-bis(diphenylphosphino)ferrocene] dichloropalladium(II) (7 mg, 0.009 mmol), and anhydrous potassium acetate (55 mg, 0.56 mmol). The flask is evacuated and purged with nitrogen three times. Anhydrous 1,4-dioxane (2 mL) was added. The reaction was heated to 100° C. and stirred for 6 hours. The reaction is added to room temperature the filtered through CELITE® with dichloromethane. The filter cake is thoroughly washed with dichloromethane. The filtered solution is concentrated in vacuo, and purification by column chromatography over silica gel afforded N-(4-((3-(4-methoxy-3-(pentyloxy)phenyl)-2-oxotetrahydropyrimidin-1(2H)-yl)methyl)-3-(4,4,5,5-tetramethyl-1,3,2-dioxaborolan-2-yl)benzyl)acetamide.

The following compounds are prepared in a similar manner as described above.

| Halide | Compound |
|---|---|
| 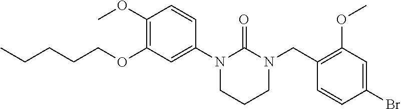 [SMILES: CCCCCOc1cc(N2CCCN(Cc3ccc(Br)cc3OC)C2=O)ccc1OC] | 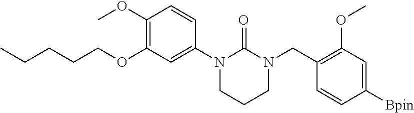 |

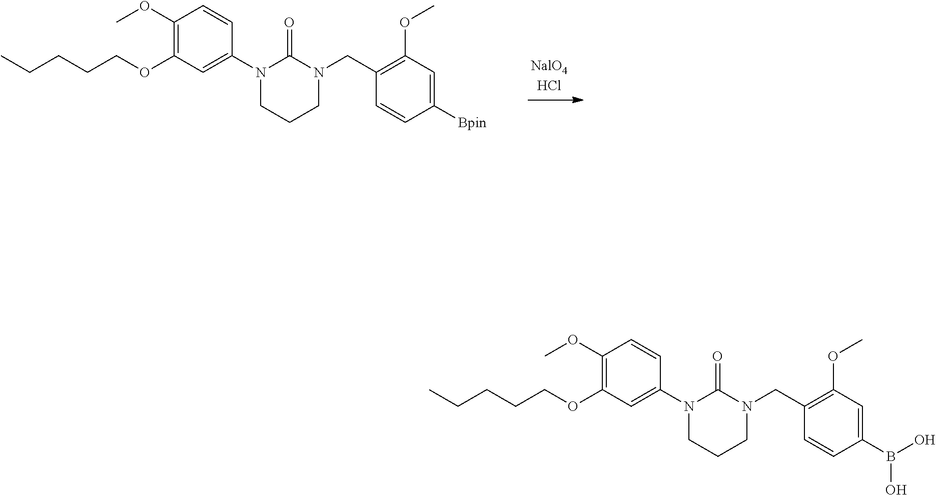

A solution of 1-(4-methoxy-3-(pentyloxy)phenyl)-3-(2-methoxy-4-(4,4,5,5-tetramethyl-1,3,2-dioxaborolan-2-yl)benzyl)tetrahydropyrimidin-2(1H)-one (250 mg, 0.46 mmol) in a 4:1 v/v mixture of tetrahydrofuran/water (5 mL) is treated with sodium periodate (298 mg, 1.39 mmol). After stirring this mixture at room temperature for 30 minutes, 1N hydrochloric acid (0.46 mL, 0.46 mmol) is added. The reaction is stirred at room temperature overnight. The reaction is diluted with water then extracted with ethyl acetate. The combined organic layers are washed with brine, then dried over anhydrous sodium sulfate, filtered, and concentrated in vacuo. Purification by column chromatography over reversed-phase C18 silica gel afforded (3-methoxy-4-((3-(4-methoxy-3-(pentyloxy)phenyl)-2-oxotetrahydropyrimidin-1(2H)-yl)methyl)phenyl)boronic acid.

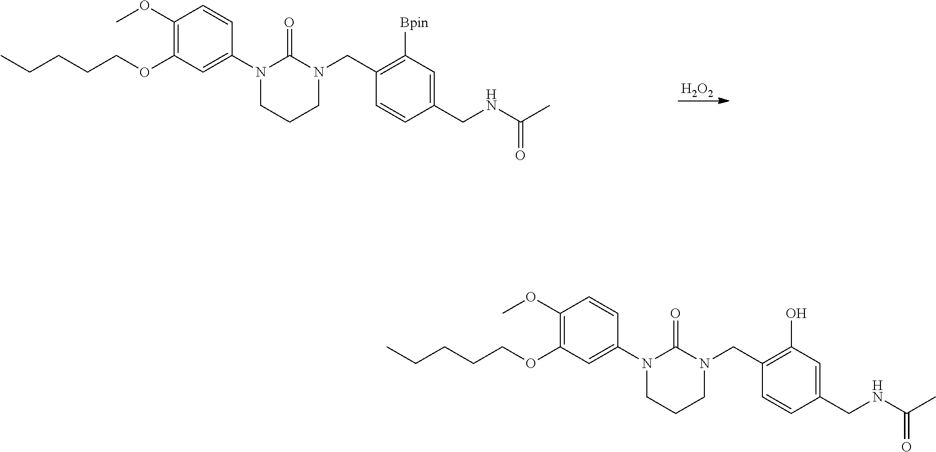

N-(4-((3-(4-methoxy-3-(pentyloxy)phenyl)-2-oxotetrahydropyrimidin-1(2H)-yl)methyl)-3-(4,4,5,5-tetramethyl-1,3,2-dioxaborolan-2-yl)benzyl)acetamide (50 mg, 0.11 mmol) is dissolved in methanol (0.2 mL) and water (0.1 mL). Solid ammonium bicarbonate (52 mg, 0.66 mmol) and 30 wt % aqueous hydrogen peroxide (0.13 mL, 1.1 mmol) are added. The reaction is stirred at room temperature for 2 hours. The reaction is cooled to 0° C. then quenched with the addition of saturated sodium sulfite solution. This mixture is extracted with ethyl acetate. The combined organic layers are dried over anhydrous sodium sulfate, filtered, and concentrated in vacuo. Purification by column chromatography of silica gel afforded N-(3-hydroxy-4-((3-(4-methoxy-3-(pentyloxy)phenyl)-2-oxotetrahydropyrimidin-1(2H)-yl)methyl)benzyl)acetamide.

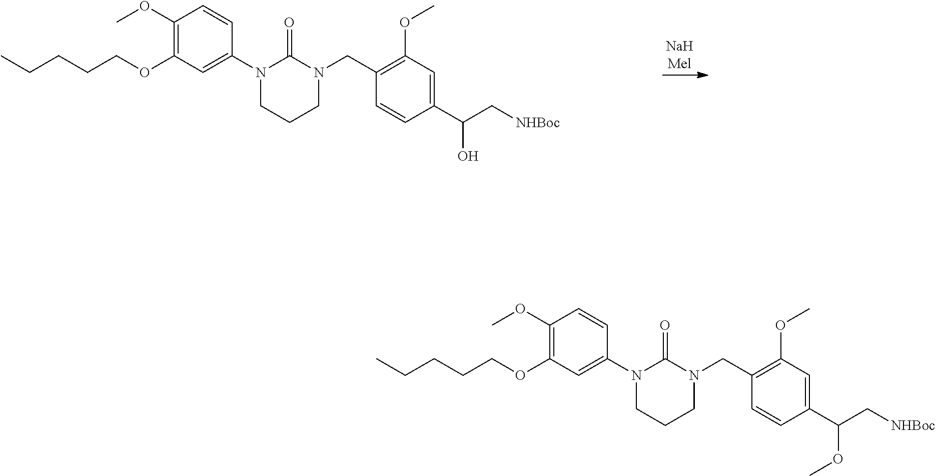 [SMILES: CCCCCOc1cc(N2CCCN(Cc3ccc(C(O)CNC(=O)OC(C)(C)C)cc3OC)C2=O)ccc1OC ; CCCCCOc1cc(N2CCCN(Cc3ccc(C(CNC(=O)OC(C)(C)C)OC)cc3OC)C2=O)ccc1OC]

A solution of tert-butyl (2-hydroxy-2-(3-methoxy-4-((3-(4-methoxy-3-(pentyloxy)phenyl)-2-oxotetrahydropyrimidin-1(2H)-yl)methyl)phenyl)ethyl)carbamate (150 mg, 0.26 mmol) in anhydrous tetrahydrofuran (3 mL) is cooled to 0° C. Sodium hydride (60 wt % in oil, 13 mg, 0.31 mmol) is added. The reaction is stirred at 0° C. for 15 minutes before adding iodomethane (18 uL, 0.29 mmol). The reaction is stirred for 6 hours while gradually warming to room temperature. Saturated aqueous ammonium chloride solution is added, then the reaction is extracted with ethyl acetate. The combined organic layers are washed with brine, then dried over anhydrous sodium sulfate, filtered, and concentrated in vacuo. Purification by column chromatography over silica gel afforded tert-butyl (2-methoxy-2-(3-methoxy-4-((3-(4-methoxy-3-(pentyloxy)phenyl)-2-oxotetrahydropyrimidin-1(2H)-yl)methyl)phenyl)ethyl)carbamate.

The following compounds are prepared in a similar manner as described above.

| Amine | Compound |
|---|---|
| 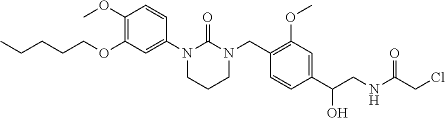 [SMILES: CCCCCOc1cc(N2CCCN(Cc3ccc(C(O)CNC(=O)CCl)cc3OC)C2=O)ccc1OC] | 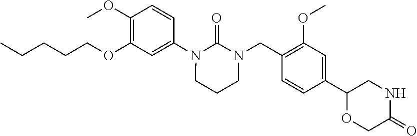 [SMILES: CCCCCOc1cc(N2CCCN(Cc3ccc(C4CNC(=O)CO4)cc3OC)C2=O)ccc1OC] |

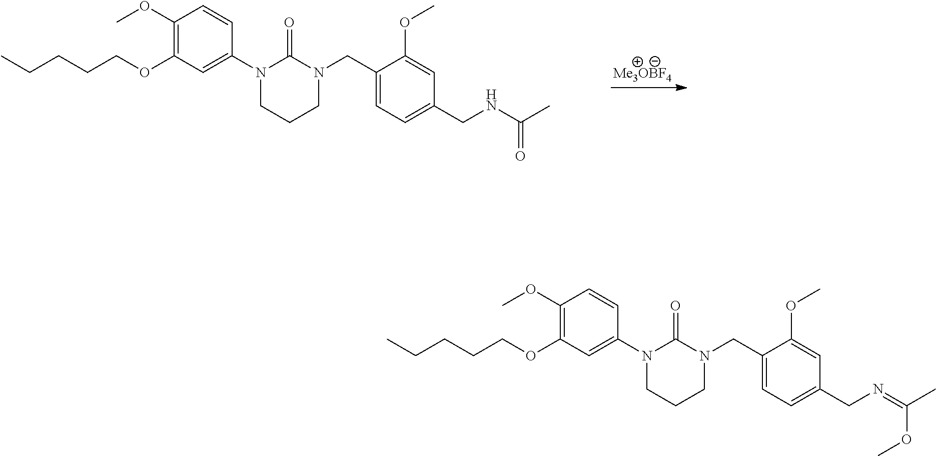 [SMILES: CCCCCOc1cc(N2CCCN(Cc3ccc(CNC(C)=O)cc3OC)C2=O)ccc1OC ; CCCCCOc1cc(N2CCCN(Cc3ccc(C/N=C(/C)OC)cc3OC)C2=O)ccc1OC]

To a suspension of N-(3-methoxy-4-((3-(4-methoxy-3-(pentyloxy)phenyl)-2-oxotetrahydropyrimidin-1(2H)-yl)methyl)benzyl)acetamide (500 mg, 1.03 mmol) in anhydrous dichloromethane (5 mL) is added trimethyloxonium tetrafluroborate (229 mg, 1.55 mmol). The reaction is stirred overnight at room temperature. Saturated aqueous ammonium chloride solution is added, then the reaction is extracted with dichloromethane. The combined organic layers are washed with brine, then dried over anhydrous magnesium sulfate, filtered, and concentrated in vacuo to afford methyl (Z)—N-(3-methoxy-4-((3-(4-methoxy-3-(pentyloxy)phenyl)-2-oxotetrahydropyrimidin-1(2H)-yl)methyl)benzyl)acetimidate.

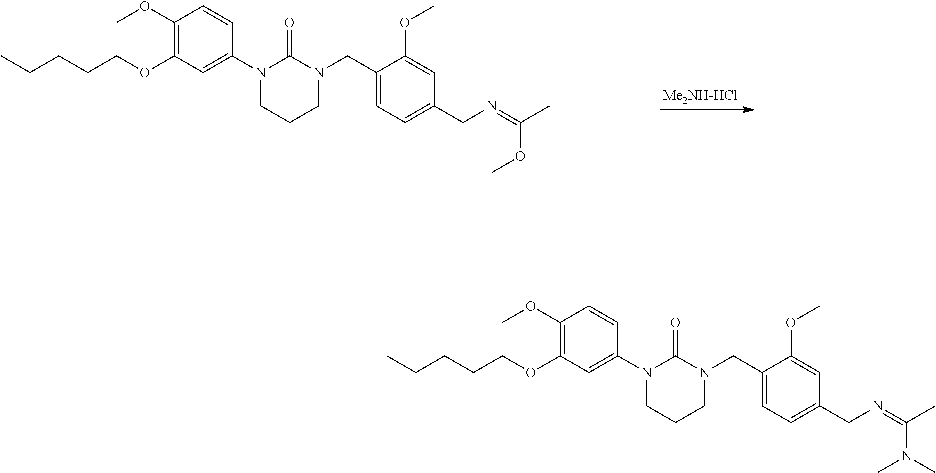 [SMILES: "Me2NH-HCl" ; CCCCCOc1cc(N2CCCN(Cc3ccc(C/N=C(/C)OC)cc3OC)C2=O)ccc1OC ; CCCCCOc1cc(N2CCCN(Cc3ccc(C/N=C(/C)N(C)C)cc3OC)C2=O)ccc1OC]

To a solution of methyl (Z)—N-(3-methoxy-4-((3-(4-methoxy-3-(pentyloxy)phenyl)-2-oxotetrahydropyrimidin-1(2H)-yl)methyl)benzyl)acetimidate (100 mg, 0.20 mmol) in anhydrous N,N-dimethylformamide (2 mL) is added anhydrous potassium carbonate (41 mg, 0.30 mmol) and dimethylamine hydrochloride (24 mg, 0.30 mmol). The reaction is heated to 60° C. and stirred for 30 minutes at this temperature. After cooling to room temperature, water is added. The mixture is extracted with dichloromethane. The combined organic layers are washed with brine, then dried over anhydrous magnesium sulfate, filtered, and concentrated in vacuo. Purification by column chromatography over reversed-phase C18 silica gel afforded (Z)—N′-(3-methoxy-4-((3-(4-methoxy-3-(pentyloxy)phenyl)-2-oxotetrahydropyrimidin-1(2H)-yl)methyl)benzyl)-N,N-dimethylacetimidamide.

The following compounds are prepared in a similar manner as described above.

| Imidate | Amine | Compound |
|---|---|---|
| 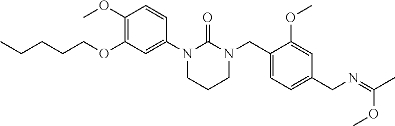 [SMILES: CCCCCOc1cc(N2CCCN(Cc3ccc(C/N=C(/C)OC)cc3OC)C2=O)ccc1OC] | MeNH₂—HCl | 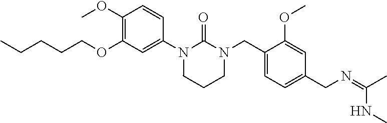 [SMILES: CCCCCOc1cc(N2CCCN(Cc3ccc(C/N=C(/C)NC)cc3OC)C2=O)ccc1OC] |

### Example 23. Compound Analysis by Surface Plasmon Resonance (SPR)

C5 inhibitor candidate compounds were synthesized according to standard methods known in the art [see, e.g. Morrison and Boyd in “Organic Chemistry”, 6^{th} edition, Prentice Hall (1992)] or as described in detail below, and analyzed using surface plasmon resonance (SPR) technology to generate data on the affinity, specificity, and kinetics of compound interactions with human C5 complement protein in real time without the need for labeling.

SensiQ FE SPR system (SensiQ Technologies, Oklahoma City, Okla.) was used to provide sensitive and accurate detection of binding of small molecules to the very large C5 protein (MW=195,000 Da). The chip was prepared by preconditioning the sensor according to the protocol of the SensiQ FE using 10 mM HCl, 50 mM NAOH and 0.1% SDS. The sensor chip was activated by using a mixture of fresh EDAC (1-ethyl-3-(-3-dimethylaminopropyl) carbodiimide) (Sigma Co., St. Louis, Mo.) and NHS (N-hydroxy succinimide) (Sigma Co., St. Louis, Mo.). Human C5 was surface immobilized to a Pioneer Biosensor chip via random amine coupling (>12,000 RU) which makes use of the N-terminus and ε-amino groups of lysine residues of the protein ligand. Immobilization was done by injecting 30-40 ug/ml C5 in 10 mM NaAc pH 4.5 onto designated channels at a rate of 10 μL/minute for 12 minutes, targeting RL>12000 RU for small molecules.

Compounds were diluted in DMSO in a format of 100-fold final concentration and 3-fold serial dilution (5 or 6 dilutions). The 100-fold compounds were transferred to one fold DMSO-free assay buffer in the 96-well test plate. The compound solution was injected at a rate of 60 μL/minute for 30-60 seconds, followed by 60-90 seconds dissociation time, buffer flushing and/or priming. Blank solution (1% DMSO assay buffer) was run for every 6 injections of compounds. Double reference by subtracting both blank channel and reference channel was applied for data processing. Titration of C5 binding compounds to the C5-immobilized biosensor chip surface led to interactions between C5 and potential binders, and the resulting changes of surface refractive index were sensitively measured by the system.

SPR data was analyzed with the managing software provided by SensiQ and equilibrium dissociation constant (K_{D}) values were determined for each compound at 37° C. Values obtained are presented in Table 28. Where a range of compound concentrations were analyzed, the lowest value obtained is presented.

TABLE 28
Surface plasmon resonance (SPR) data

| Compound | K_{D} |
|---|---|
| Number | (nM) |
| SC0064 | 11.13 |
| SC0066 | 43.70 |

### Example 24. Compound Analysis by Red Blood Cell (RBC) Hemolysis Assay

Sheep red blood cells coated with rabbit anti-sheep erythrocyte antiserum (EA cells; Complement Technology, Tyler, Tex.) were used to assay compound inhibitory activity of the classical complement activation pathway. Briefly, the EA cells were washed once and resuspended in the same volume of GVB++ buffer (Complement Technology, Tyler, Tex.). 25 μL of EA cells were then distributed into each well of 384-well tissue culture plates using Apricot iPipette Pro (Apricot Designs; Covina, Calif.). Compounds were tested in 10 points of final concentrations ranging from 16.67 μM to 1.65 μM in a 6-fold titration series. Compounds were dispensed into 384-well plates from 6.7 mM and 3.35 μM DMSO working stocks using an HP Digital Dispenser (HP; Corvallis, Oreg.). The reactions also contained 1.5% (v/v) C5-depleted human serum (Complement Technology). Hemolysis was induced by addition of human C5 (Complement Technology) at a concentration of 0.5 nM and plates were incubated for 1 hour at 37° C. in a cell culture incubator. The extent of hemolysis was measured by ability of released hemoglobin to catalyze luminol in the presence of hydrogen peroxide. Luminescence was then measured using a plate reader.

Luminescence measurements were used to prepare a dose-response curve. From the curve, the half maximal inhibitory concentration (IC₅₀) for each compound was determined, where the IC₅₀ represents the concentration of each compound needed to reduce red blood cell hemolysis by half. Results are presented in Table 29. Numbers in parenthesis following the compound number indicate alternate enantiomers (as distinguished by retention time during chromatographical separation).

TABLE 29
Red blood cell (RBC) hemolysis assay data

| Compound | IC₅₀ |
|---|---|
| Number | (nM) |
| SC0001 | 8.4 |
| SC0016(1) | 8.4 |
| SC0002 | 8.7 |
| SC0003 | 9.9 |
| SC0004 | 10.5 |
| SC0005 | 11.2 |
| SC0006 | 11.2 |
| SC0016(2) | 12.8 |
| SC0007 | 13.6 |
| SC0072 | 14.2 |
| SC0044 | 14.9 |
| SC0008 | 18.5 |
| SC0009 | 18.5 |
| SC0060 | 22.5 |
| SC0052 | 23.7 |
| SC0064 | 23.7 |
| SC0053 | 24.2 |
| SC0045 | 24.3 |
| SC0010 | 24.7 |
| SC0011 | 25.2 |
| SC0017 | 28.2 |
| SC0012 | 28.5 |
| SC0046 | 33.0 |
| SC0054 | 34.8 |
| SC0047 | 36.2 |
| SC0055 | 38.2 |
| SC0065 | 39.0 |
| SC0066 | 39.7 |
| SC0056 | 42.5 |
| SC0018 | 44.7 |
| SC0013 | 46.7 |
| SC0057 | 47.5 |
| SC0058 | 50.8 |
| SC0048 | 52.3 |
| SC0028 | 53.7 |
| SC0019 | 55.9 |
| SC0059 | 57.7 |
| SC0061 | 58.6 |
| SC0067 | 59.0 |
| SC0024 | 59.3 |
| SC0025 | 59.4 |
| SC0026 | 59.7 |
| SC0027 | 65.1 |
| SC0020 | 66.8 |
| SC0049 | 68.1 |
| SC0021 | 71.5 |
| SC0050 | 76.2 |
| SC0068 | 77.2 |
| SC0029 | 84.5 |
| SC0030 | 94.5 |
| SC0031 | 96.0 |
| SC0032 | 99.8 |
| SC0033 | 101.2 |
| SC0051 | 110.6 |
| SC0023 | 111.2 |
| SC0034 | 114.8 |
| SC0035 | 122.0 |
| SC0036 | 122.1 |
| SC0037 | 123.6 |
| SC0069 | 128.6 |
| SC0038 | 129.2 |
| SC0022 | 139.8 |
| SC0014 | 146.2 |
| SC0062 | 173.3 |
| SC0039 | 195.7 |
| SC0040 | 208.1 |
| SC0015 | 222.1 |
| SC0041 | 251.3 |
| SC0042 | 258.3 |
| SC0043 | 337.4 |
| SC0070 | 398.2 |
| SC0071 | 870.2 |
| SC0063 | 985.0 |

### Example 25. Compound Analysis by Liquid Chromatography-Mass Spectrometry (LC-MS)

Compounds were analyzed by Liquid chromatography-mass spectrometry (LC-MS) after synthesis to confirm mass-to-charge ratio (m/z). Analytical LCMS was performed by Waters Aquity SDS using a linear gradient of 5% to 100% B over a 5 minute gradient, and UV visualization with a diode array detector. The column used was a C18 Aquity UPLC BEH, 2.1 mm i.d. by 50 mm length, 1.7 μM with flow rate of 0.6 ml/min. Mobile phase A was water and mobile phase B was acetonitrile (0.1% TFA). Results are shown in Table 30. Numbers in parenthesis following the compound number indicate alternate enantiomers (as distinguished by retention time during chromatographical separation).

TABLE 30
LCMS assay data

| | m/z found |
|---|---|
| Compound Number | [M + H] |
| SC0001 | 547.6 |
| SC0002 | 607.7 |
| SC0003 | 576.6 |
| SC0004 | 577.7 |
| SC0005 | 607.7 |
| SC0006 | 563.6 |
| SC0007 | 620.7 |
| SC0008 | 521.6 |
| SC0009 | 607.7 |
| SC0010 | 436.5 |
| SC0011 | 584.6 |
| SC0012 | 563.6 |
| SC0013 | 527.6 |
| SC0014 | 521.6 |
| SC0015 | 437.5 |
| SC0016(1) | 461.2 |
| SC0016(2) | 461.2 |
| SC0017 | 512.2 |
| SC0018 | 445.2 |
| SC0019 | 485.2 |
| SC0020 | 445.2 |
| SC0021 | 445.2 |
| SC0022 | 477.4 |
| SC0023 | 553.3 |
| SC0024 | 519.2 |
| SC0025 | 546.2 |
| SC0026 | 576.2 |
| SC0027 | 523.1 |
| SC0028 | 518.2 |
| SC0029 | 588.3 |
| SC0030 | 532.2 |
| SC0031 | 525.5 |
| SC0032 | 589.5 |
| SC0033 | 511.4 |
| SC0034 | 460.1 |
| SC0035 | 512.5 |
| SC0036 | 556.1 |
| SC0037 | 577.2 |
| SC0038 | 589.5 |
| SC0039 | 512.5 |
| SC0040 | 542.4 |
| SC0041 | 561.5 |
| SC0042 | 511.5 |
| SC0043 | 561.5 |
| SC0044 | 474.2 |
| SC0045 | 578.2 |
| SC0046 | 488.2 |
| SC0047 | 532.2 |
| SC0048 | 502.2 |
| SC0049 | 532.2 |
| SC0050 | 541.3 |
| SC0051 | 544.2 |
| SC0052 | 502.1 |
| SC0053 | 555.1 |
| SC0054 | 565.1 |
| SC0055 | 559.1 |
| SC0056 | 532.2 |
| SC0057 | 568.2 |
| SC0058 | 447.3 |
| SC0059 | 475.1 |
| SC0060 | 443.3 |
| SC0061 | 443.4 |
| SC0062 | 504.9 |
| SC0063 | 431.4 |
| SC0064 | 439.5 |
| SC0065 | 534.5 |
| SC0066 | 456.0 |
| SC0067 | 544.4 |
| SC0068 | 493.4 |
| SC0069 | 551.1 |
| SC0070 | 472.3 |
| SC0071 | 626.5 |
| SC0072 | 461.2 |

### Example 26. Synthesis of Substituted Cyclic Urea Compounds and Intermediates

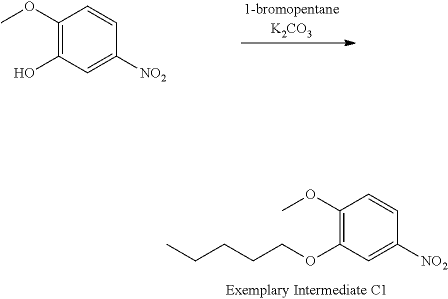 [SMILES: COc1ccc([N+](=O)[O-])cc1O ; CCCCCOc1cc([N+](=O)[O-])ccc1OC]

A solution which included a phenol reactant (2-methoxy-5-nitrophenol, 100.0 g, 0.59 mol) and a bromide reactant (1-bromopentane, 117.2 g, 0.76 mol, 1.3 eq) in a reaction solvent (N,N-dimethylformamide, 1.0 L) is provided. Potassium carbonate (122.5 g, 0.89 mol, 1.5 eq) is added at room temperature. The mixture is heated to 80° C. and stirred overnight. The reaction mixture is cooled to room temperature, diluted with water (3.0 L), then extracted with ethyl acetate (3×2.0 L). The combined organic phases are washed with brine (3×3.0 L), dried over anhydrous sodium sulfate, then filtered and concentrated in vacuo to a volume of about 300 mL. The residue is diluted with hexane (1.0 L) and stirred for 10 minutes to precipitate a white solid. The solids are collected by filtration and dried under vacuum to afford a compound, Exemplary Intermediate C1 (1-methoxy-4-nitro-2-(pentyloxy)benzene, 119.7 g, 85% yield).

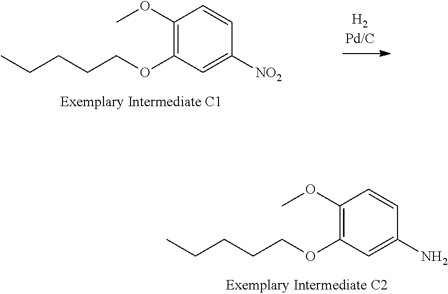 [SMILES: CCCCCOc1cc([N+](=O)[O-])ccc1OC ; CCCCCOc1cc(N)ccc1OC]

Exemplary Intermediate C1 (1-methoxy-4-nitro-2-(pentyloxy)benzene, 119.5 g, 0.50 mol) is dissolved in methanol (1.5 L), and 10 wt % palladium on carbon (10 g) is added. The mixture is stirred overnight under an atmosphere of hydrogen. The reaction mixture is filtered through Celite, and the filter bed is washed with methanol (500 mL). The filtered solution is concentrated to dryness to afford Exemplary Intermediate C2 (4-methoxy-3-(pentyloxy)aniline, 95.0 g, 91% yield).

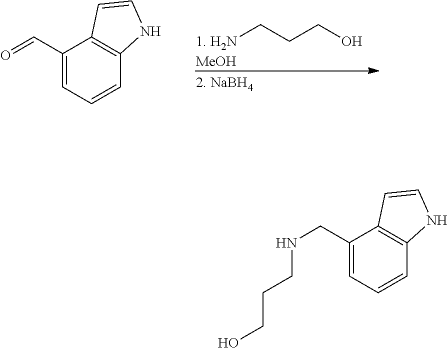

A solution of 1H-pyrrolo[2,3-b]pyridine-4-carboxaldehyde (1.00 g, 6.85 mmol) and 3-aminopropanol (0.51 g, 6.85 mmol) in methanol (10 mL) is heated at 70° C. for 1 h then cooled to 0° C. Sodium borohydride (0.52 g, 13.7 mmol) is added, and the mixture is stirred for 30 minutes. The reaction is quenched with water and extracted three times with dichloromethane. The combined organic layers are washed with brine then dried over sodium sulfate and filtered. The filtered solution is concentrated in vacuo. Trituration of the resulting oil with ethyl acetate afforded 3-(((1H-pyrrolo[2,3-b]pyridine-4-yl)methyl)amino)propan-1-ol as a white solid (1.12 g, 80% yield).

The following compounds are prepared in a similar manner as described above.

| Aldehyde | Alcohol | Compound |
|---|---|---|
| 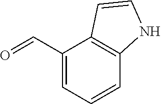 [SMILES: O=Cc1cccc2[nH]ccc12] | 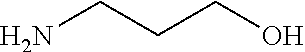 | 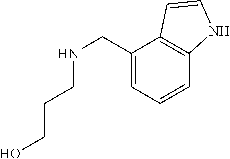 [SMILES: OCCCNCc1cccc2[nH]ccc12] |
| 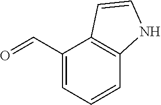 [SMILES: O=Cc1cccc2[nH]ccc12] | 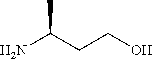 | 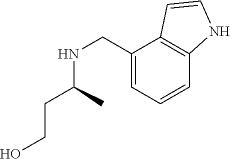 [SMILES: C[C@@H](CCO)NCc1cccc2[nH]ccc12] |
| 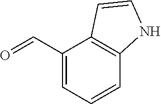 [SMILES: O=Cc1cccc2[nH]ccc12] | 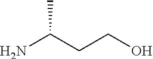 | 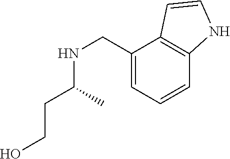 [SMILES: C[C@H](CCO)NCc1cccc2[nH]ccc12] |
| 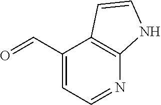 [SMILES: O=Cc1ccnc2[nH]ccc12] | 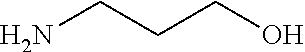 | 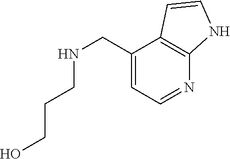 [SMILES: OCCCNCc1ccnc2[nH]ccc12] |
| 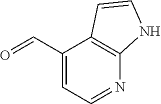 [SMILES: O=Cc1ccnc2[nH]ccc12] | 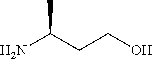 | 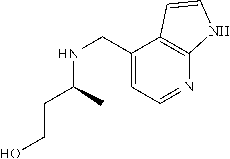 [SMILES: C[C@@H](CCO)NCc1ccnc2[nH]ccc12] |
| 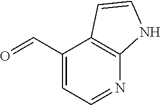 [SMILES: O=Cc1ccnc2[nH]ccc12] | 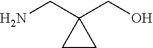 | 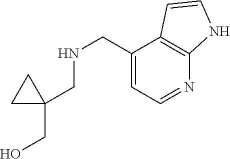 [SMILES: OCC1(CNCc2ccnc3[nH]ccc23)CC1] |
| 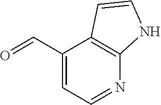 [SMILES: O=Cc1ccnc2[nH]ccc12] | 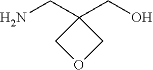 | 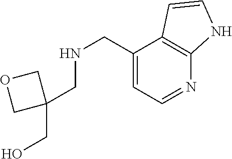 [SMILES: OCC1(CNCc2ccnc3[nH]ccc23)COC1] |
| 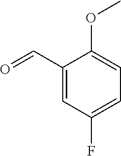 | 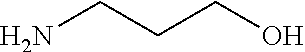 | 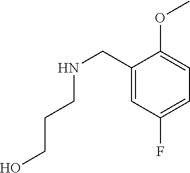 |
| 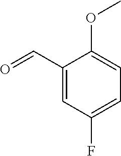 | 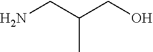 | 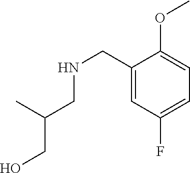 [SMILES: COc1ccc(F)cc1CNCC(C)CO] |
| 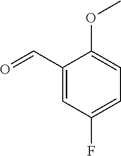 | 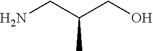 | 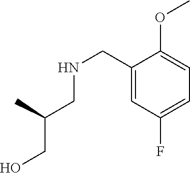 [SMILES: COc1ccc(F)cc1CNC[C@H](C)CO] |
| 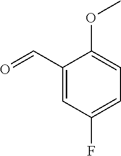 | 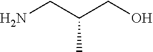 | 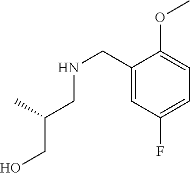 [SMILES: COc1ccc(F)cc1CNC[C@@H](C)CO] |
| 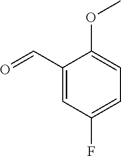 | 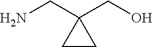 | 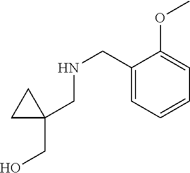 [SMILES: COc1ccccc1CNCC1(CO)CC1] |
| 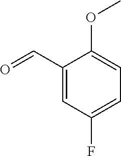 | 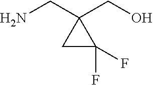 | 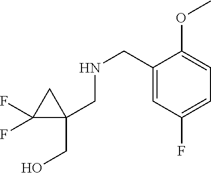 [SMILES: COc1ccc(F)cc1CNCC1(CO)CC1(F)F] |
| 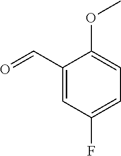 | 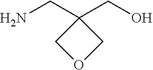 | 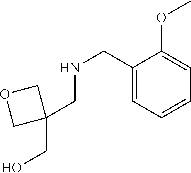 [SMILES: COc1ccccc1CNCC1(CO)COC1] |
| 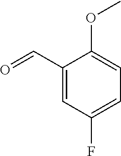 | 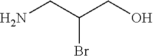 | 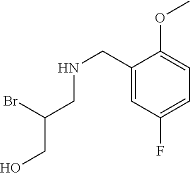 [SMILES: COc1ccc(F)cc1CNCC(Br)CO] |
| 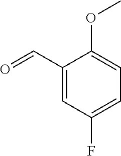 | 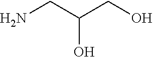 | 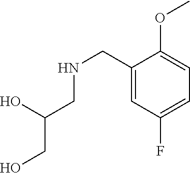 [SMILES: COc1ccc(F)cc1CNCC(O)CO] |
| 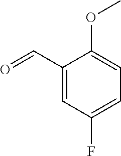 | 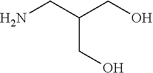 | 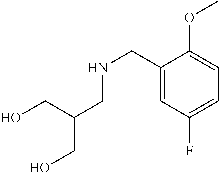 [SMILES: COc1ccc(F)cc1CNCC(CO)CO] |
| 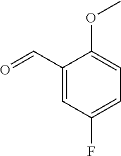 | 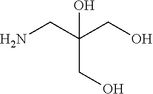 | 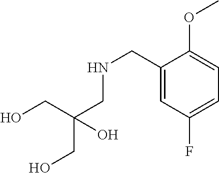 [SMILES: COc1ccc(F)cc1CNCC(O)(CO)CO] |
| 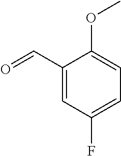 | 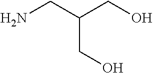 | 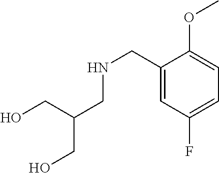 [SMILES: COc1ccc(F)cc1CNCC(CO)CO] |
| 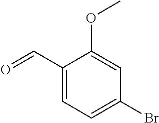 | 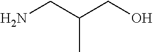 | 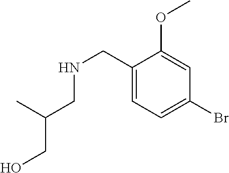 [SMILES: COc1cc(Br)ccc1CNCC(C)CO] |
| 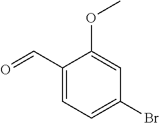 | 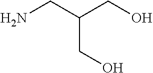 | 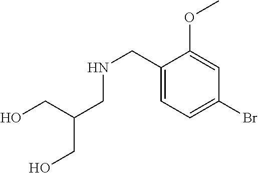 [SMILES: COc1cc(Br)ccc1CNCC(CO)CO] |

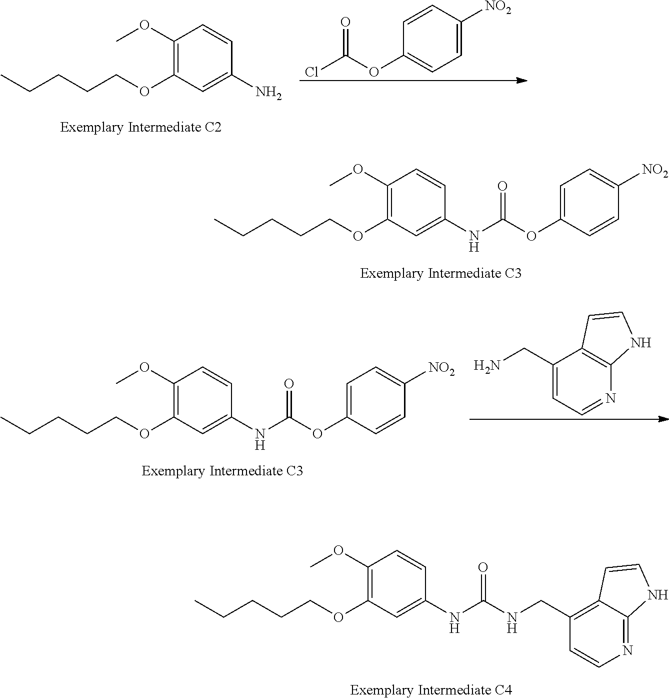 [SMILES: CCCCCOc1cc(N)ccc1OC ; O=C(Cl)Oc1ccc([N+](=O)[O-])cc1 ; CCCCCOc1cc(NC(=O)Oc2ccc([N+](=O)[O-])cc2)ccc1OC ; CCCCCOc1cc(NC(=O)Oc2ccc([N+](=O)[O-])cc2)ccc1OC ; NCc1ccnc2[nH]ccc12 ; CCCCCOc1cc(NC(=O)NCc2ccnc3[nH]ccc23)ccc1OC]

A solution of 4-nitrophenyl chloroformate (2.1 g, 10 mmol) in anhydrous tetrahydrofuran (10 mL) is cooled to 0° C., then treated with the dropwise addition Exemplary Intermediate C2 (4-methoxy-3-(pentyloxy)aniline; 2.0 g, 9.6 mmol) dissolved in 20 mL tetrahydrofuran, over 10 min. The mixture is stirred at room temperature for 12 hr. The precipitate is collected by filtration and washed with tert-butyl methyl ether/petroleum ether mixture to afford Exemplary Intermediate C3 (4-nitrophenyl N-(4-methoxy-3-(pentyloxy)phenyl) carbamate; 2.8 g, 78% yield).

To a solution of Exemplary Intermediate C3 (1.38 g, 3.69 mmol) in N,N-dimethylformamide (40 mL) is added 1H-pyrrolo[2,3-b]pyridin-4-ylmethanamine hydrochloride (812 mg, 4.42 mmol), followed by triethylamine (1.4 mL, 10.0 mmol). The mixture is stirred at room temperature overnight. The reaction mixture is diluted with ethyl acetate and water to form a precipitate. The organic layer suspension is washed three times with water, then the solid is isolated from the organic layer by filtration to afford Exemplary Intermediate C4 (1-(4-methoxy-3-pentoxyphenyl)-3-(1H-pyrrolo[2,3-b]pyridin-4-ylmethyl)urea; 698 mg, 50% yield). The organic filtrate is dried over a phase separator and concentrated in vacuo. The residue is triturated with ethyl acetate-cyclohexane mixture to afford additional Exemplary Intermediate C4 (207 mg, 15% yield).

The following compounds are prepared in a similar manner as Exemplary Intermediate C4, as described above.

| Aniline | Amine |
|---|---|
| 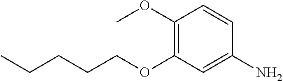 | 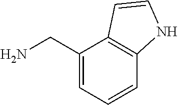 [SMILES: NCc1cccc2[nH]ccc12] |
| 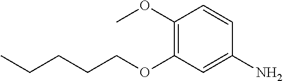 | 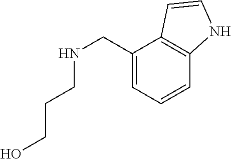 [SMILES: OCCCNCc1cccc2[nH]ccc12] |
| 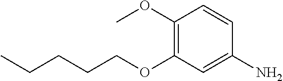 | 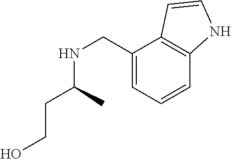 [SMILES: C[C@@H](CCO)NCc1cccc2[nH]ccc12] |
| 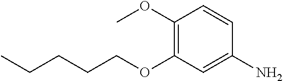 | 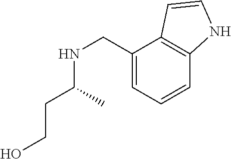 [SMILES: C[C@H](CCO)NCc1cccc2[nH]ccc12] |
| 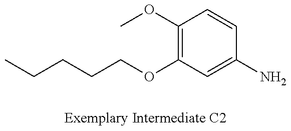 | 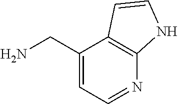 [SMILES: NCc1ccnc2[nH]ccc12] |
| 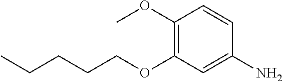 | 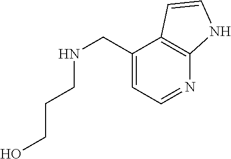 [SMILES: OCCCNCc1ccnc2[nH]ccc12] |
| 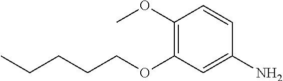 | 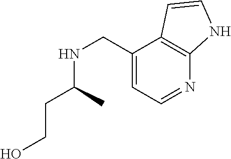 [SMILES: C[C@@H](CCO)NCc1ccnc2[nH]ccc12] |
| 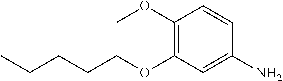 | 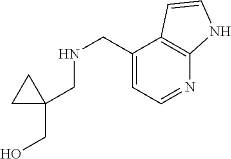 [SMILES: OCC1(CNCc2ccnc3[nH]ccc23)CC1] |
| 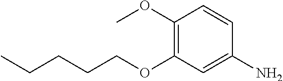 | 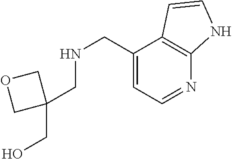 [SMILES: OCC1(CNCc2ccnc3[nH]ccc23)COC1] |
| 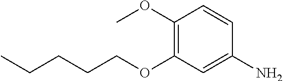 | 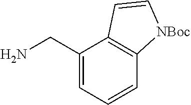 [SMILES: CC(C)(C)OC(=O)n1ccc2c(CN)cccc21] |
| 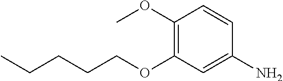 | 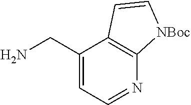 [SMILES: CC(C)(C)OC(=O)n1ccc2c(CN)ccnc21] |
| 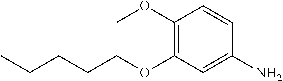 | 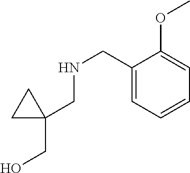 [SMILES: COc1ccccc1CNCC1(CO)CC1] |
| 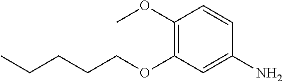 | 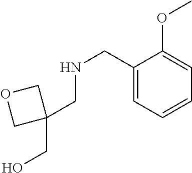 [SMILES: COc1ccccc1CNCC1(CO)COC1] |
| 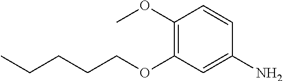 | 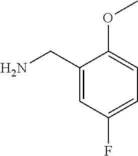 |
| 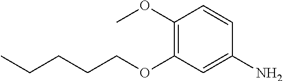 | 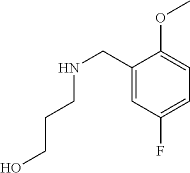 |
| 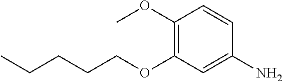 | 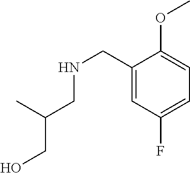 [SMILES: COc1ccc(F)cc1CNCC(C)CO] |
| 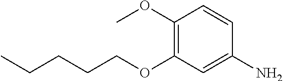 | 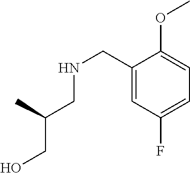 [SMILES: COc1ccc(F)cc1CNC[C@H](C)CO] |
| 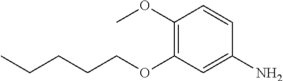 | 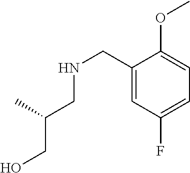 [SMILES: COc1ccc(F)cc1CNC[C@@H](C)CO] |
| 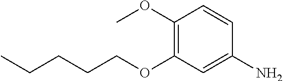 | 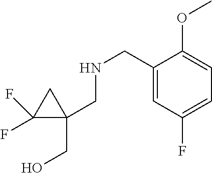 [SMILES: COc1ccc(F)cc1CNCC1(CO)CC1(F)F] |
| 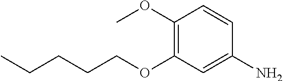 | 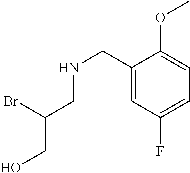 [SMILES: COc1ccc(F)cc1CNCC(Br)CO] |
| 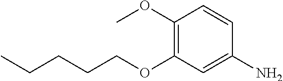 | 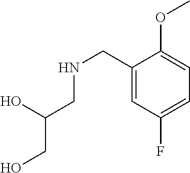 [SMILES: COc1ccc(F)cc1CNCC(O)CO] |
| 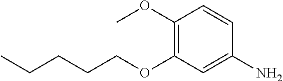 | 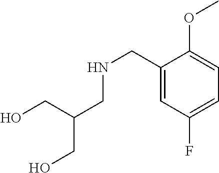 [SMILES: COc1ccc(F)cc1CNCC(CO)CO] |
| 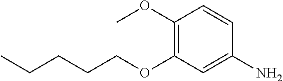 | 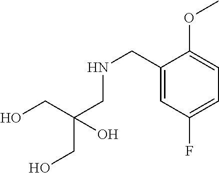 [SMILES: COc1ccc(F)cc1CNCC(O)(CO)CO] |
| 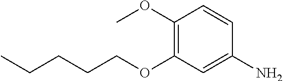 | 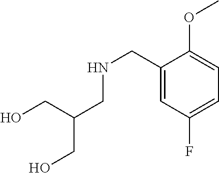 [SMILES: COc1ccc(F)cc1CNCC(CO)CO] |
| 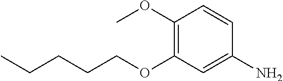 | 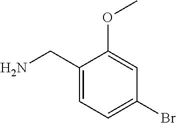 |
| 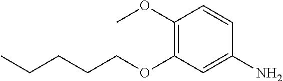 | 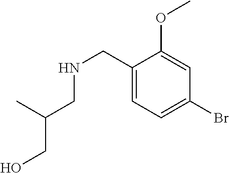 [SMILES: COc1cc(Br)ccc1CNCC(C)CO] |
| 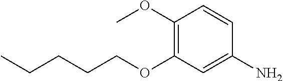 | 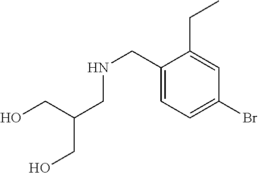 [SMILES: CCc1cc(Br)ccc1CNCC(CO)CO] |

Compound

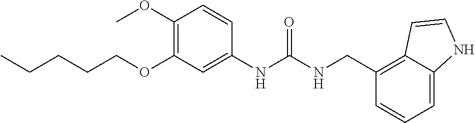 [SMILES: CCCCCOc1cc(NC(=O)NCc2cccc3[nH]ccc23)ccc1OC]

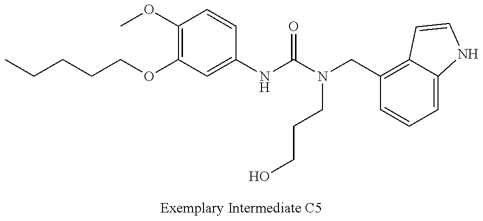 [SMILES: CCCCCOc1cc(NC(=O)N(CCCO)Cc2cccc3[nH]ccc23)ccc1OC]

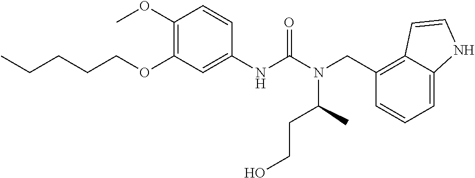 [SMILES: CCCCCOc1cc(NC(=O)N(Cc2cccc3[nH]ccc23)[C@@H](C)CCO)ccc1OC]

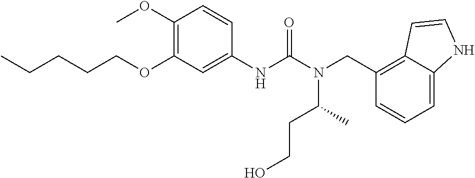 [SMILES: CCCCCOc1cc(NC(=O)N(Cc2cccc3[nH]ccc23)[C@H](C)CCO)ccc1OC]

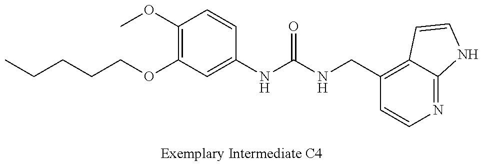 [SMILES: CCCCCOc1cc(NC(=O)NCc2ccnc3[nH]ccc23)ccc1OC]

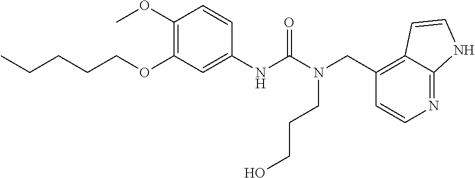 [SMILES: CCCCCOc1cc(NC(=O)N(CCCO)Cc2ccnc3[nH]ccc23)ccc1OC]

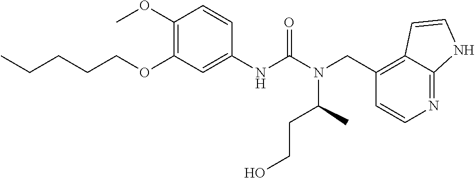 [SMILES: CCCCCOc1cc(NC(=O)N(Cc2ccnc3[nH]ccc23)[C@@H](C)CCO)ccc1OC]

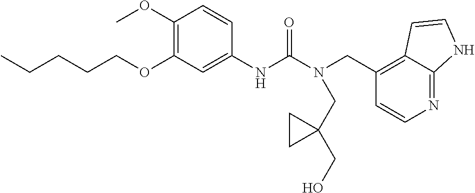 [SMILES: CCCCCOc1cc(NC(=O)N(Cc2ccnc3[nH]ccc23)CC2(CO)CC2)ccc1OC]

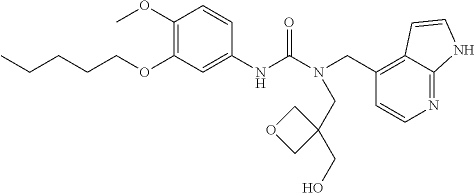 [SMILES: CCCCCOc1cc(NC(=O)N(Cc2ccnc3[nH]ccc23)CC2(CO)COC2)ccc1OC]

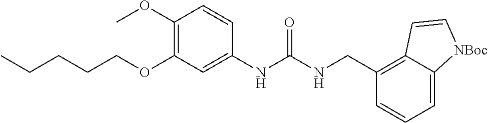 [SMILES: CCCCCOc1cc(NC(=O)NCc2cccc3c2ccn3C(=O)OC(C)(C)C)ccc1OC]

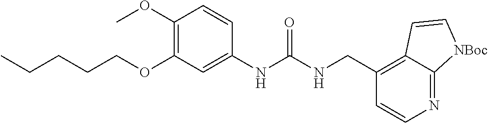 [SMILES: CCCCCOc1cc(NC(=O)NCc2ccnc3c2ccn3C(=O)OC(C)(C)C)ccc1OC]

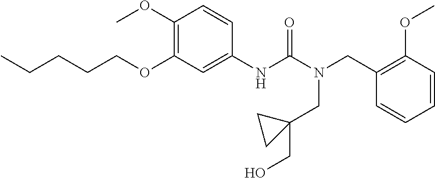 [SMILES: CCCCCOc1cc(NC(=O)N(Cc2ccccc2OC)CC2(CO)CC2)ccc1OC]

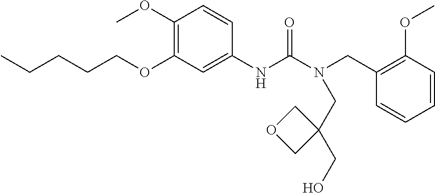 [SMILES: CCCCCOc1cc(NC(=O)N(Cc2ccccc2OC)CC2(CO)COC2)ccc1OC]

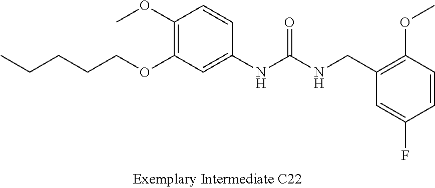 [SMILES: CCCCCOc1cc(NC(=O)NCc2cc(F)ccc2OC)ccc1OC]

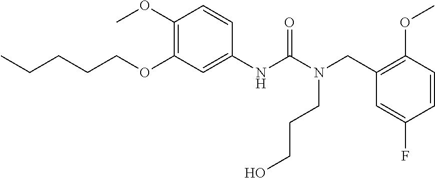 [SMILES: CCCCCOc1cc(NC(=O)N(CCCO)Cc2cc(F)ccc2OC)ccc1OC]

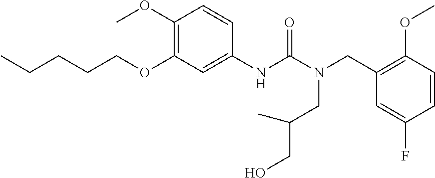 [SMILES: CCCCCOc1cc(NC(=O)N(Cc2cc(F)ccc2OC)CC(C)CO)ccc1OC]

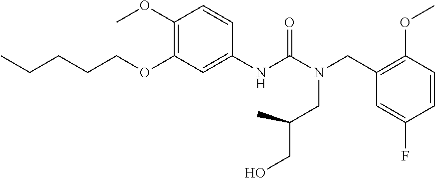 [SMILES: CCCCCOc1cc(NC(=O)N(Cc2cc(F)ccc2OC)C[C@H](C)CO)ccc1OC]

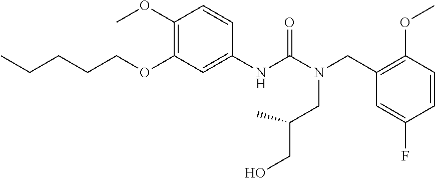 [SMILES: CCCCCOc1cc(NC(=O)N(Cc2cc(F)ccc2OC)C[C@@H](C)CO)ccc1OC]

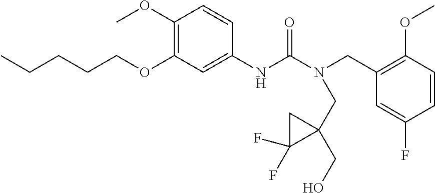 [SMILES: CCCCCOc1cc(NC(=O)N(Cc2cc(F)ccc2OC)CC2(CO)CC2(F)F)ccc1OC]

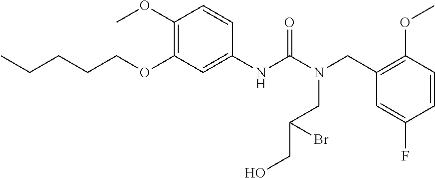 [SMILES: CCCCCOc1cc(NC(=O)N(Cc2cc(F)ccc2OC)CC(Br)CO)ccc1OC]

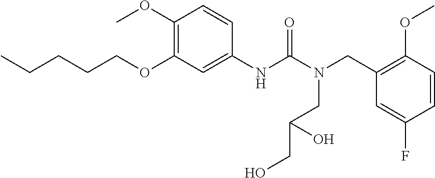 [SMILES: CCCCCOc1cc(NC(=O)N(Cc2cc(F)ccc2OC)CC(O)CO)ccc1OC]

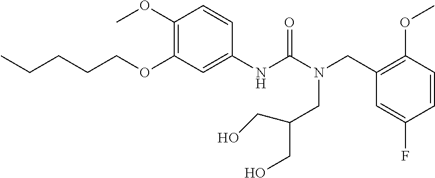 [SMILES: CCCCCOc1cc(NC(=O)N(Cc2cc(F)ccc2OC)CC(CO)CO)ccc1OC]

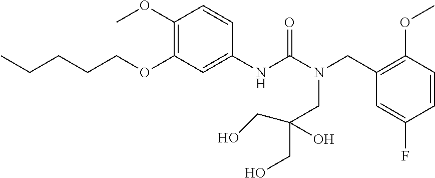 [SMILES: CCCCCOc1cc(NC(=O)N(Cc2cc(F)ccc2OC)CC(O)(CO)CO)ccc1OC]

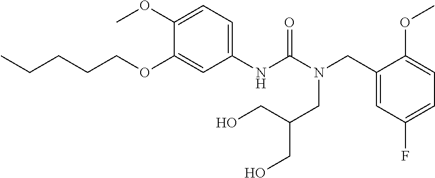 [SMILES: CCCCCOc1cc(NC(=O)N(Cc2cc(F)ccc2OC)CC(CO)CO)ccc1OC]

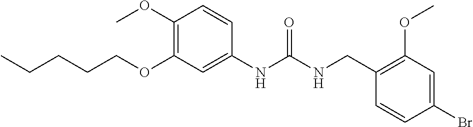 [SMILES: CCCCCOc1cc(NC(=O)NCc2ccc(Br)cc2OC)ccc1OC]

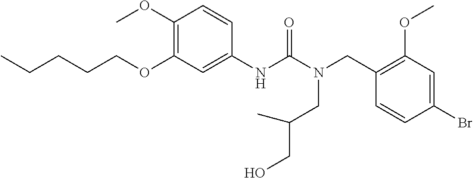 [SMILES: CCCCCOc1cc(NC(=O)N(Cc2ccc(Br)cc2OC)CC(C)CO)ccc1OC]

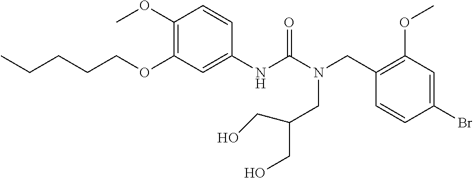 [SMILES: CCCCCOc1cc(NC(=O)N(Cc2ccc(Br)cc2OC)CC(CO)CO)ccc1OC]

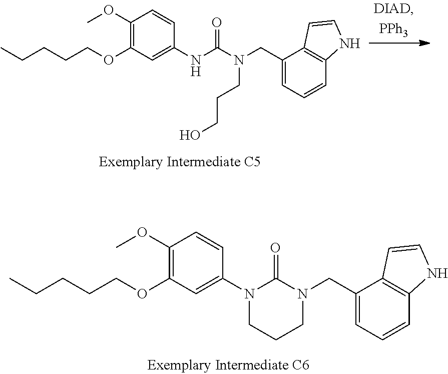 [SMILES: CCCCCOc1cc(NC(=O)N(CCCO)Cc2cccc3[nH]ccc23)ccc1OC ; CCCCCOc1cc(N2CCCN(Cc3cccc4[nH]ccc34)C2=O)ccc1OC]

To a stirring solution of Exemplary Intermediate C5 (1-((1H-indol-4-yl)methyl)-1-(3-hydroxypropyl)-3-(4-methoxy-3-(pentyloxy)phenyl)urea; 1.20 g, 2.75 mmol) and triphenylphosphine (0.86 g, 3.3 mmol) in anhydrous tetrahydrofuran (10 mL), cooled to at 0° C., is added diisopropyl azodicarboxylate (0.83 g, 4.12 mmol). The resulting solution is warmed slowly to room temperature while stirring for 8 h. The reaction is quenched with water and extracted with ethyl acetate. The combined organic layers are washed with brine then dried over sodium sulfate and filtered. The filtered solution is concentrated in vacuo. The residue is purified by column chromatography over silica gel using 5% (v/v) methanol in dichloromethane (with 0.1% v/v ammonium hydroxide additive) as eluent to afford Exemplary Intermediate C6 (1-((1H-indol-4-yl)methyl)-3-(4-methoxy-3-(pentyloxy)phenyl) tetrahydropyrimidin-2(1H)-one; 905 mg, 65% yield).

The following compounds are prepared in a similar manner as Exemplary Intermediate C6 as described above.

| Open Urea | Cyclic Urea |
|---|---|
| 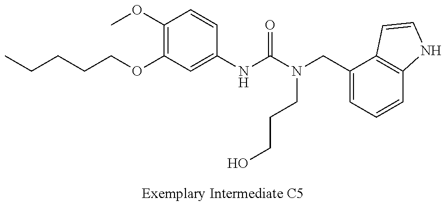 [SMILES: CCCCCOc1cc(NC(=O)N(CCCO)Cc2cccc3[nH]ccc23)ccc1OC] | 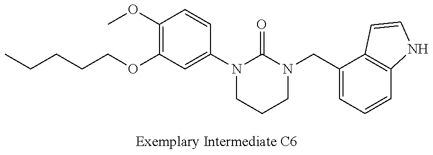 [SMILES: CCCCCOc1cc(N2CCCN(Cc3cccc4[nH]ccc34)C2=O)ccc1OC] |
| 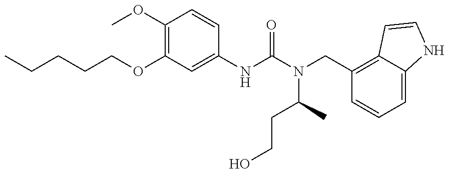 [SMILES: CCCCCOc1cc(NC(=O)N(Cc2cccc3[nH]ccc23)[C@@H](C)CCO)ccc1OC] | 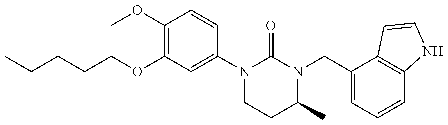 [SMILES: CCCCCOc1cc(N2CC[C@H](C)N(Cc3cccc4[nH]ccc34)C2=O)ccc1OC] |
| 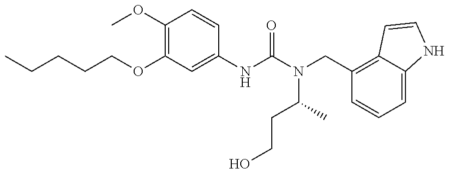 [SMILES: CCCCCOc1cc(NC(=O)N(Cc2cccc3[nH]ccc23)[C@H](C)CCO)ccc1OC] | 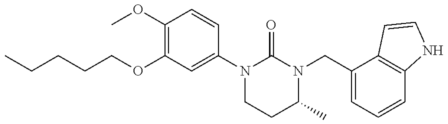 [SMILES: CCCCCOc1cc(N2CC[C@@H](C)N(Cc3cccc4[nH]ccc34)C2=O)ccc1OC] |
| 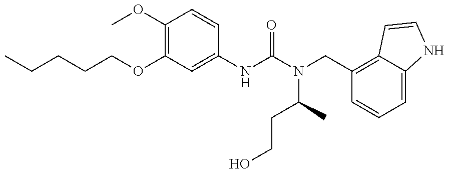 [SMILES: CCCCCOc1cc(NC(=O)N(Cc2cccc3[nH]ccc23)[C@@H](C)CCO)ccc1OC] | 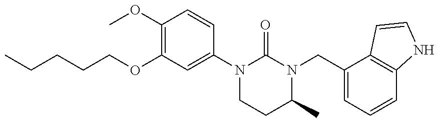 [SMILES: CCCCCOc1cc(N2CC[C@H](C)N(Cc3cccc4[nH]ccc34)C2=O)ccc1OC] |
| 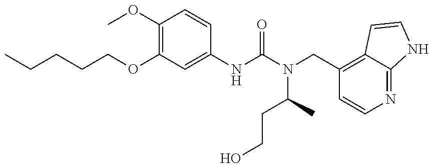 [SMILES: CCCCCOc1cc(NC(=O)N(Cc2ccnc3[nH]ccc23)[C@@H](C)CCO)ccc1OC] | 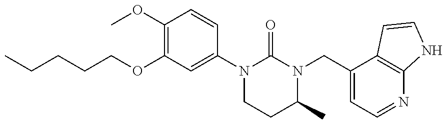 [SMILES: CCCCCOc1cc(N2CC[C@H](C)N(Cc3ccnc4[nH]ccc34)C2=O)ccc1OC] |
| 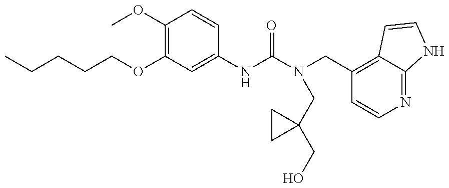 [SMILES: CCCCCOc1cc(NC(=O)N(Cc2ccnc3[nH]ccc23)CC2(CO)CC2)ccc1OC] | 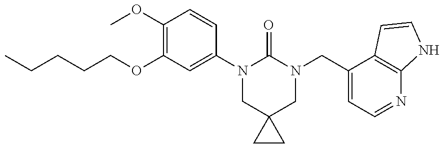 [SMILES: CCCCCOc1cc(N2CC3(CC3)CN(Cc3ccnc4[nH]ccc34)C2=O)ccc1OC] |
| 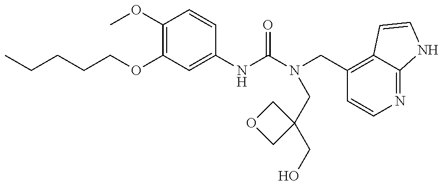 [SMILES: CCCCCOc1cc(NC(=O)N(Cc2ccnc3[nH]ccc23)CC2(CO)COC2)ccc1OC] | 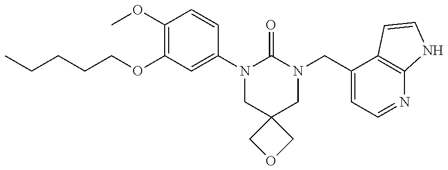 [SMILES: CCCCCOc1cc(N2CC3(COC3)CN(Cc3ccnc4[nH]ccc34)C2=O)ccc1OC] |
| 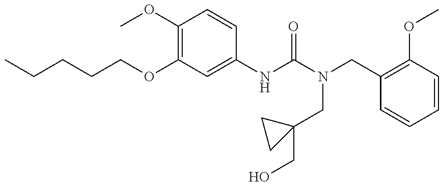 [SMILES: CCCCCOc1cc(NC(=O)N(Cc2ccccc2OC)CC2(CO)CC2)ccc1OC] | 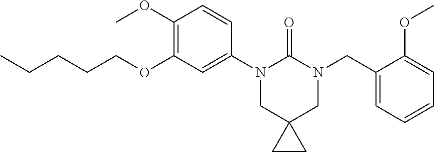 [SMILES: CCCCCOc1cc(N2CC3(CC3)CN(Cc3ccccc3OC)C2=O)ccc1OC] |
| 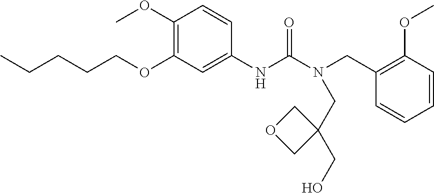 [SMILES: CCCCCOc1cc(NC(=O)N(Cc2ccccc2OC)CC2(CO)COC2)ccc1OC] | 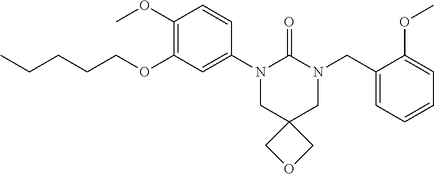 [SMILES: CCCCCOc1cc(N2CC3(COC3)CN(Cc3ccccc3OC)C2=O)ccc1OC] |
| 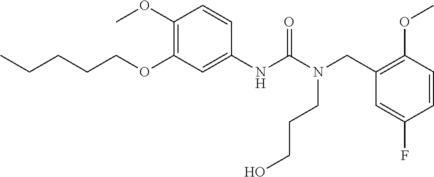 [SMILES: CCCCCOc1cc(NC(=O)N(CCCO)Cc2cc(F)ccc2OC)ccc1OC] | 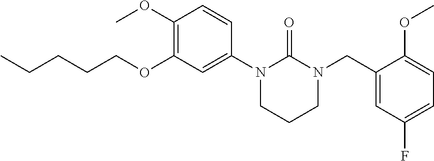 [SMILES: CCCCCOc1cc(N2CCCN(Cc3cc(F)ccc3OC)C2=O)ccc1OC] |
| 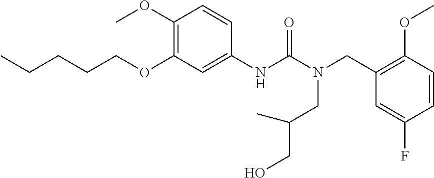 [SMILES: CCCCCOc1cc(NC(=O)N(Cc2cc(F)ccc2OC)CC(C)CO)ccc1OC] | 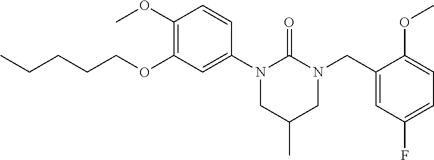 [SMILES: CCCCCOc1cc(N2CC(C)CN(Cc3cc(F)ccc3OC)C2=O)ccc1OC] |
| 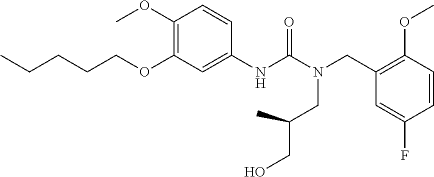 [SMILES: CCCCCOc1cc(NC(=O)N(Cc2cc(F)ccc2OC)C[C@H](C)CO)ccc1OC] | 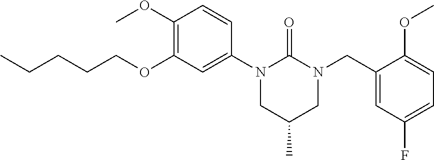 [SMILES: CCCCCOc1cc(N2C[C@@H](C)CN(Cc3cc(F)ccc3OC)C2=O)ccc1OC] |
| 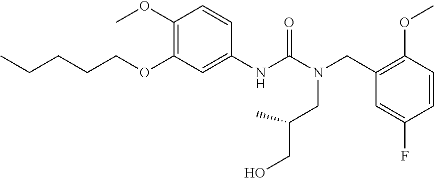 [SMILES: CCCCCOc1cc(NC(=O)N(Cc2cc(F)ccc2OC)C[C@@H](C)CO)ccc1OC] | 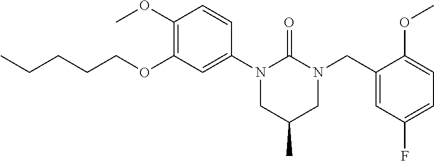 [SMILES: CCCCCOc1cc(N2C[C@H](C)CN(Cc3cc(F)ccc3OC)C2=O)ccc1OC] |
| 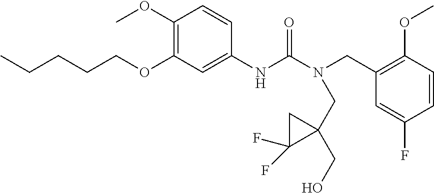 [SMILES: CCCCCOc1cc(NC(=O)N(Cc2cc(F)ccc2OC)CC2(CO)CC2(F)F)ccc1OC] | 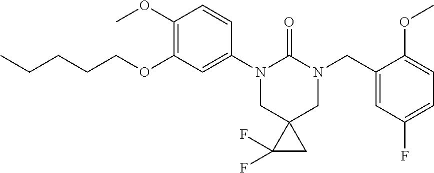 [SMILES: CCCCCOc1cc(N2CC3(CN(Cc4cc(F)ccc4OC)C2=O)CC3(F)F)ccc1OC] |
| 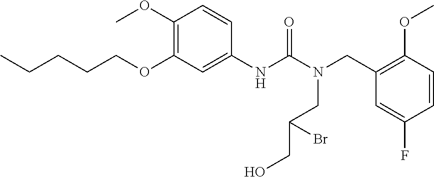 [SMILES: CCCCCOc1cc(NC(=O)N(Cc2cc(F)ccc2OC)CC(Br)CO)ccc1OC] | 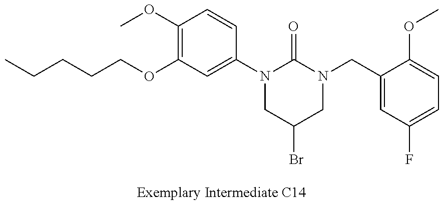 [SMILES: CCCCCOc1cc(N2CC(Br)CN(Cc3cc(F)ccc3OC)C2=O)ccc1OC] |
| 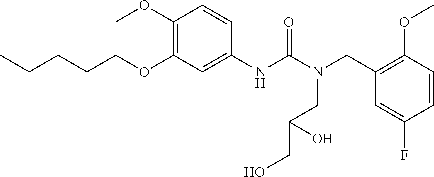 [SMILES: CCCCCOc1cc(NC(=O)N(Cc2cc(F)ccc2OC)CC(O)CO)ccc1OC] | 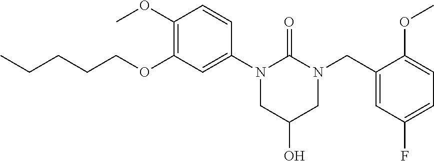 [SMILES: CCCCCOc1cc(N2CC(O)CN(Cc3cc(F)ccc3OC)C2=O)ccc1OC] |
| 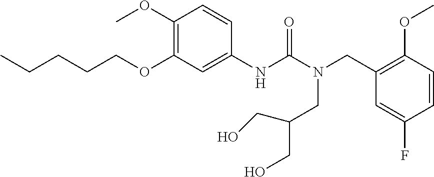 [SMILES: CCCCCOc1cc(NC(=O)N(Cc2cc(F)ccc2OC)CC(CO)CO)ccc1OC] | 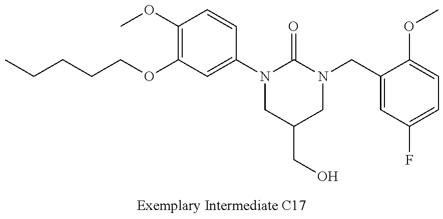 [SMILES: CCCCCOc1cc(N2CC(CO)CN(Cc3cc(F)ccc3OC)C2=O)ccc1OC] |
| 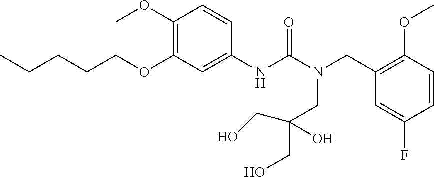 [SMILES: CCCCCOc1cc(NC(=O)N(Cc2cc(F)ccc2OC)CC(O)(CO)CO)ccc1OC] | 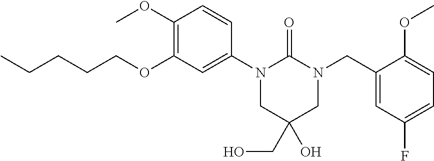 [SMILES: CCCCCOc1cc(N2CC(O)(CO)CN(Cc3cc(F)ccc3OC)C2=O)ccc1OC] |
| 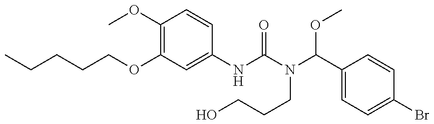 [SMILES: CCCCCOc1cc(NC(=O)N(CCCO)C(OC)c2ccc(Br)cc2)ccc1OC] | 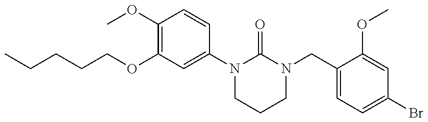 [SMILES: CCCCCOc1cc(N2CCCN(Cc3ccc(Br)cc3OC)C2=O)ccc1OC] |
| 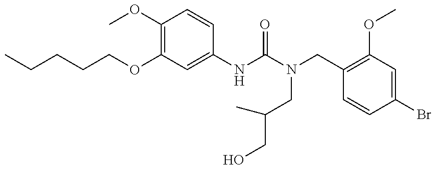 [SMILES: CCCCCOc1cc(NC(=O)N(Cc2ccc(Br)cc2OC)CC(C)CO)ccc1OC] | 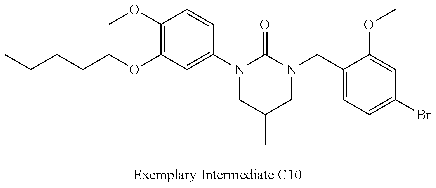 [SMILES: CCCCCOc1cc(N2CC(C)CN(Cc3ccc(Br)cc3OC)C2=O)ccc1OC] |
| 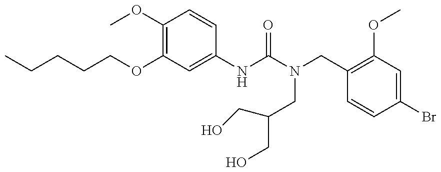 [SMILES: CCCCCOc1cc(NC(=O)N(Cc2ccc(Br)cc2OC)CC(CO)CO)ccc1OC] | 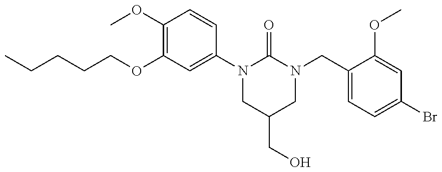 [SMILES: CCCCCOc1cc(N2CC(CO)CN(Cc3ccc(Br)cc3OC)C2=O)ccc1OC] |

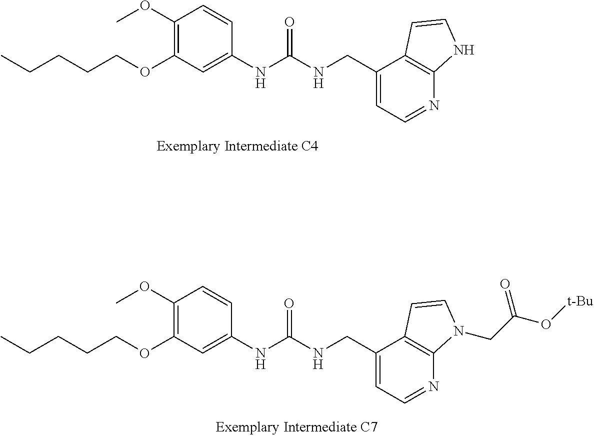 [SMILES: CCCCCOc1cc(NC(=O)NCc2ccnc3[nH]ccc23)ccc1OC ; CCCCCOc1cc(NC(=O)NCc2ccnc3c2ccn3CC(=O)OC(C)(C)C)ccc1OC]

To a solution of Exemplary Intermediate C4 (1-(4-methoxy-3-pentoxyphenyl)-3-(1H-pyrrolo[2,3-b]pyrindin-4-ylmethyl)urea; 905 mg, 2.37 mmol) in N,N-dimethylfomamide (22 mL) is added sodium hydride (60 wt % in mineral oil, 189 mg, 4.73 mmol) in portions at room temperature. The mixture is stirred at room temperature for 10 min then cooled to 0° C., and a solution of tert-butyl bromoacetate (0.52 mL, 3.55 mmol) in N,N-dimethylformamide (3 mL) was added dropwise. The reaction is slowly warmed to room temperature and stirred for 3.5 h. Water is slowly added to the reaction, and the resulting mixture is extracted twice with ethyl acetate. The combined organic layers are washed with brine, dried over sodium sulfate, and concentrated in vacuo. The residue is purified by column chromatography over silica gel (dichloromethane/methanol: 100/0 to 97/3) to afford Exemplary Intermediate C7 (tert-butyl 2-[4-[[(4-methoxy-3-pentoxyphenyl)carbamoylamino]methyl]pyrrolo[2,3-b]pyridin-1-yl]acetate; 810 mg, 69% yield).

The following compounds are prepared in a similar manner as Exemplary Intermediate C7, as described above.

| Heterocycle | Electrophile |
|---|---|
| 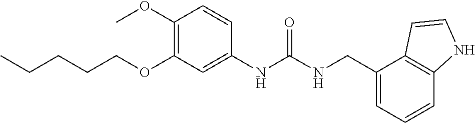 [SMILES: CCCCCOc1cc(NC(=O)NCc2cccc3[nH]ccc23)ccc1OC] | 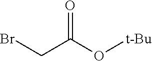 |
| 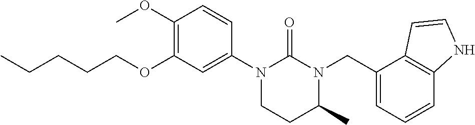 [SMILES: CCCCCOc1cc(N2CC[C@H](C)N(Cc3cccc4[nH]ccc34)C2=O)ccc1OC] | 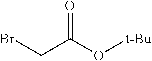 |
| 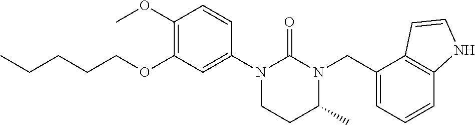 [SMILES: CCCCCOc1cc(N2CC[C@@H](C)N(Cc3cccc4[nH]ccc34)C2=O)ccc1OC] | 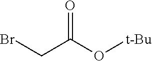 |
| 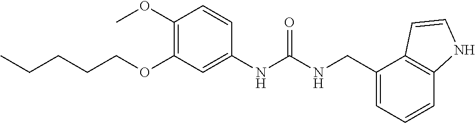 [SMILES: CCCCCOc1cc(NC(=O)NCc2cccc3[nH]ccc23)ccc1OC] | 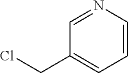 |
| 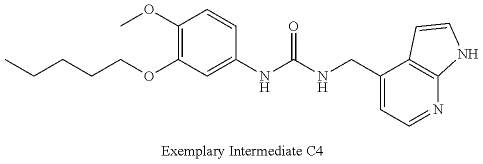 [SMILES: CCCCCOc1cc(NC(=O)NCc2ccnc3[nH]ccc23)ccc1OC] | 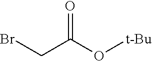 |
| 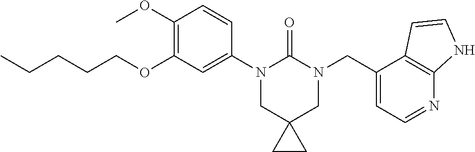 [SMILES: CCCCCOc1cc(N2CC3(CC3)CN(Cc3ccnc4[nH]ccc34)C2=O)ccc1OC] | 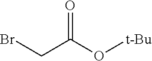 |
| 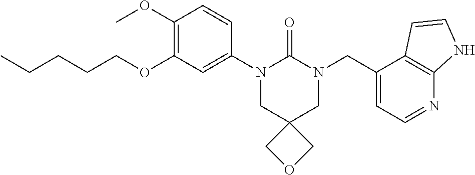 [SMILES: CCCCCOc1cc(N2CC3(COC3)CN(Cc3ccnc4[nH]ccc34)C2=O)ccc1OC] | 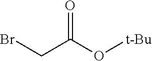 |

Compound

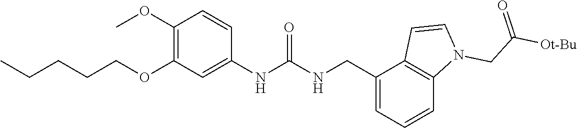 [SMILES: CCCCCOc1cc(NC(=O)NCc2cccc3c2ccn3CC(=O)OC(C)(C)C)ccc1OC]

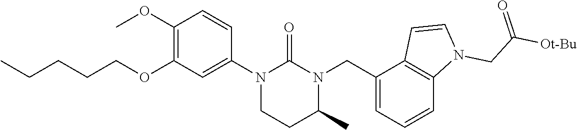 [SMILES: CCCCCOc1cc(N2CC[C@H](C)N(Cc3cccc4c3ccn4CC(=O)OC(C)(C)C)C2=O)ccc1OC]

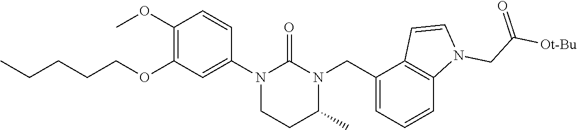 [SMILES: CCCCCOc1cc(N2CC[C@@H](C)N(Cc3cccc4c3ccn4CC(=O)OC(C)(C)C)C2=O)ccc1OC]

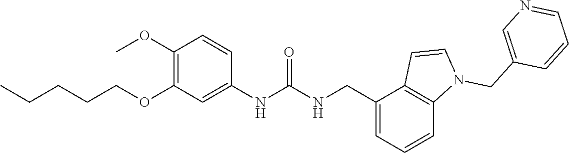 [SMILES: CCCCCOc1cc(NC(=O)NCc2cccc3c2ccn3Cc2cccnc2)ccc1OC]

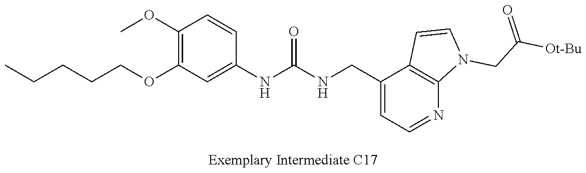 [SMILES: CCCCCOc1cc(NC(=O)NCc2ccnc3c2ccn3CC(=O)OC(C)(C)C)ccc1OC]

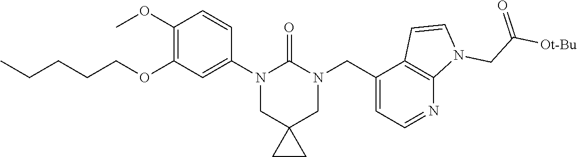 [SMILES: CCCCCOc1cc(N2CC3(CC3)CN(Cc3ccnc4c3ccn4CC(=O)OC(C)(C)C)C2=O)ccc1OC]

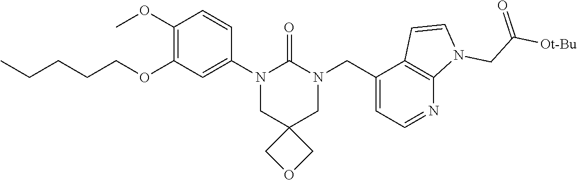 [SMILES: CCCCCOc1cc(N2CC3(COC3)CN(Cc3ccnc4c3ccn4CC(=O)OC(C)(C)C)C2=O)ccc1OC]

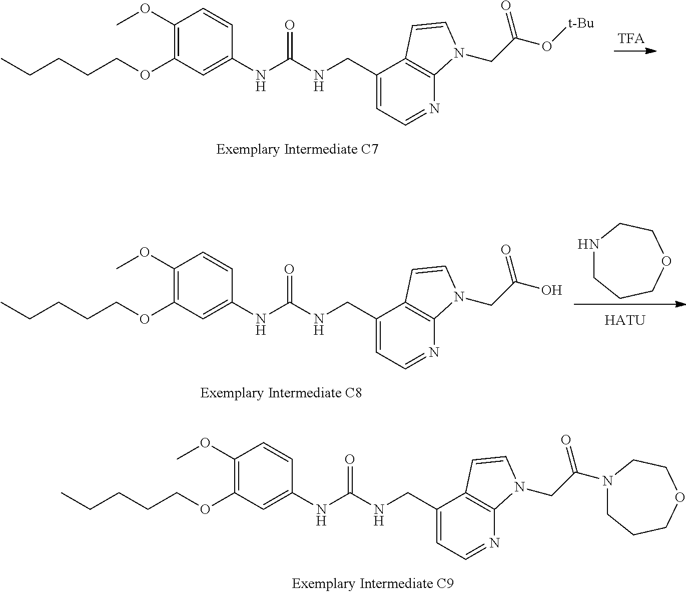

Trifluoroacetic acid (5.1 mL) is added Exemplary Intermediate C7 (tert-butyl 2-[4-[[(4-methoxy-3-pentoxyphenyl)carbamoylamino]methyl]pyrrolo[2,3-b]pyridin-1-yl]acetate; 400 mg, 0.81 mmol) at 0° C. The reaction mixture is stirred at 0° C. for 10 min, then warmed to room temperature and stirred for 2 h. The volatiles are removed in vacuo. Diethyl ether is added to the residue, and the resulting suspension was sonicated. The ether is decanted, and the remaining solids are further triturated with methanol to afford Exemplary Intermediate C8 (2-[4-[[(4-methoxy-3-pentoxyphenyl)carbamoylamino]methyl]pyrrolo[2,3-b]pyridin-1-yl]acetic acid; 100 mg, 28% yield) as white solid.

Exemplary Intermediate C8 (100 mg, 0.21 mmol) is dissolved in N,N-dimethylformamide (2.8 mL), then HATU (80 mg, 0.21 mmol), 1,4-oxazepane (0.05 mL, 0.42 mmol), and N,N-diisopropylethylamine (0.07 mL, 0.42 mmol) are added. Reaction mixture is stirred at room temperature for 2 h. Water is added to reaction mixture, and it is extracted three times with ethyl acetate. The organic layers are dried over a phase separator and concentrated in vacuo. The residue is purified by column chromatography over silica (dichloromethane/methanol: 100/0 to 97/3) to afford Exemplary Intermediate C9 (1-(4-methoxy-3-pentoxyphenyl)-3-[[1-[2-(1,4-oxazepan-4-yl)-2-oxoethyl]pyrrolo[2,3-b]pyridin-4-yl]methyl] urea; 100 mg, 90% yield).

The following compounds are prepared in a similar manner as Exemplary Intermediate C9, as described above.

| Ester | Amine |
|---|---|
| 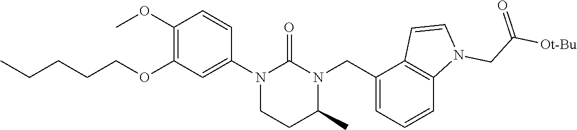 [SMILES: CCCCCOc1cc(N2CC[C@H](C)N(Cc3cccc4c3ccn4CC(=O)OC(C)(C)C)C2=O)ccc1OC] | 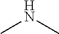 |
| 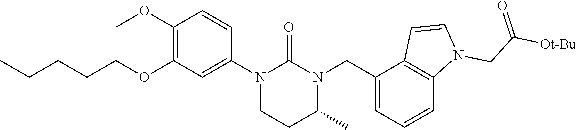 [SMILES: CCCCCOc1cc(N2CC[C@@H](C)N(Cc3cccc4c3ccn4CC(=O)OC(C)(C)C)C2=O)ccc1OC] | 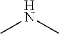 |
| 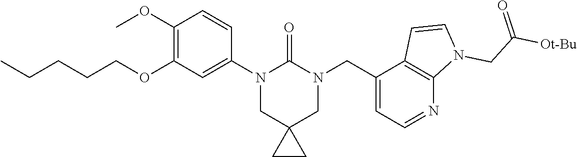 [SMILES: CCCCCOc1cc(N2CC3(CC3)CN(Cc3ccnc4c3ccn4CC(=O)OC(C)(C)C)C2=O)ccc1OC] | 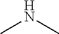 |
| 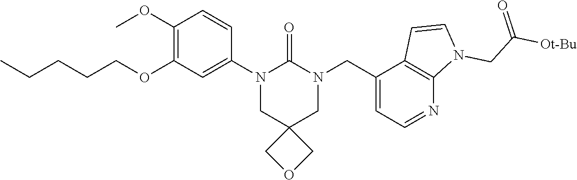 [SMILES: CCCCCOc1cc(N2CC3(COC3)CN(Cc3ccnc4c3ccn4CC(=O)OC(C)(C)C)C2=O)ccc1OC] | 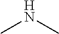 |
| 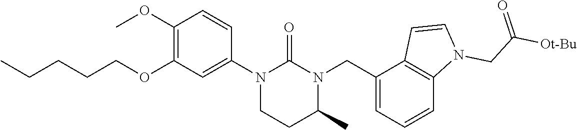 [SMILES: CCCCCOc1cc(N2CC[C@H](C)N(Cc3cccc4c3ccn4CC(=O)OC(C)(C)C)C2=O)ccc1OC] | 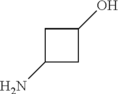 |
| 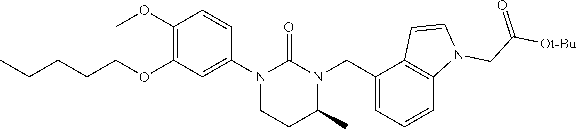 [SMILES: CCCCCOc1cc(N2CC[C@H](C)N(Cc3cccc4c3ccn4CC(=O)OC(C)(C)C)C2=O)ccc1OC] | 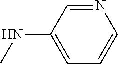 |
| 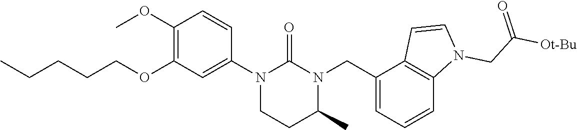 [SMILES: CCCCCOc1cc(N2CC[C@H](C)N(Cc3cccc4c3ccn4CC(=O)OC(C)(C)C)C2=O)ccc1OC] | 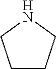 |
| 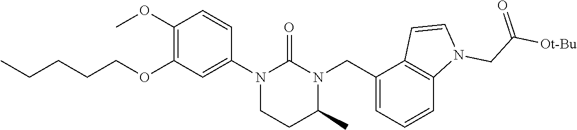 [SMILES: CCCCCOc1cc(N2CC[C@H](C)N(Cc3cccc4c3ccn4CC(=O)OC(C)(C)C)C2=O)ccc1OC] | 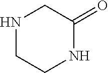 |
| 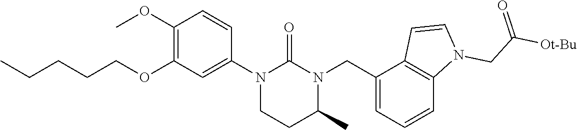 [SMILES: CCCCCOc1cc(N2CC[C@H](C)N(Cc3cccc4c3ccn4CC(=O)OC(C)(C)C)C2=O)ccc1OC] | 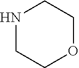 |
| 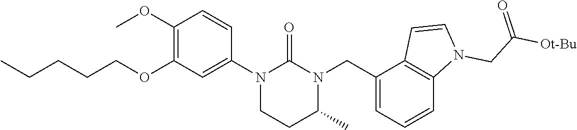 [SMILES: CCCCCOc1cc(N2CC[C@@H](C)N(Cc3cccc4c3ccn4CC(=O)OC(C)(C)C)C2=O)ccc1OC] | 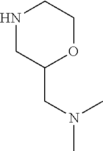 |
| 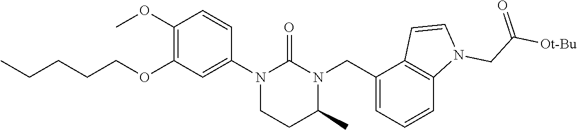 [SMILES: CCCCCOc1cc(N2CC[C@H](C)N(Cc3cccc4c3ccn4CC(=O)OC(C)(C)C)C2=O)ccc1OC] | 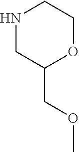 |
| 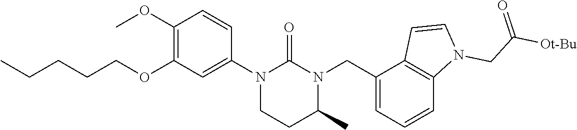 [SMILES: CCCCCOc1cc(N2CC[C@H](C)N(Cc3cccc4c3ccn4CC(=O)OC(C)(C)C)C2=O)ccc1OC] | 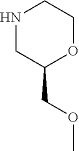 |
| 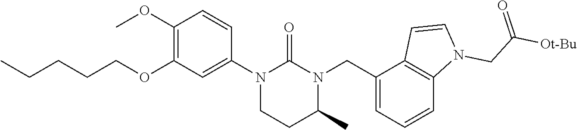 [SMILES: CCCCCOc1cc(N2CC[C@H](C)N(Cc3cccc4c3ccn4CC(=O)OC(C)(C)C)C2=O)ccc1OC] | 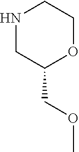 |
| 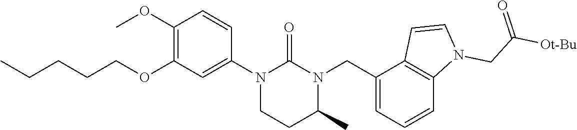 [SMILES: CCCCCOc1cc(N2CC[C@H](C)N(Cc3cccc4c3ccn4CC(=O)OC(C)(C)C)C2=O)ccc1OC] | 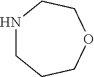 |

Compound

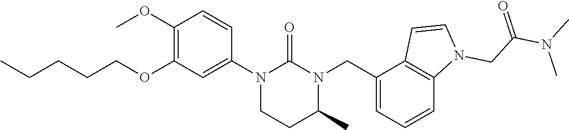 [SMILES: CCCCCOc1cc(N2CC[C@H](C)N(Cc3cccc4c3ccn4CC(=O)N(C)C)C2=O)ccc1OC]

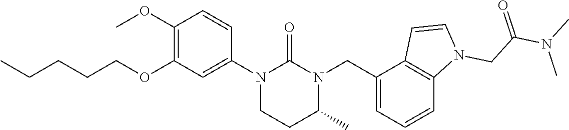 [SMILES: CCCCCOc1cc(N2CC[C@@H](C)N(Cc3cccc4c3ccn4CC(=O)N(C)C)C2=O)ccc1OC]

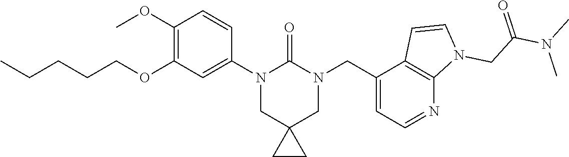 [SMILES: CCCCCOc1cc(N2CC3(CC3)CN(Cc3ccnc4c3ccn4CC(=O)N(C)C)C2=O)ccc1OC]

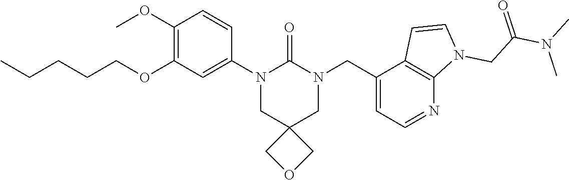 [SMILES: CCCCCOc1cc(N2CC3(COC3)CN(Cc3ccnc4c3ccn4CC(=O)N(C)C)C2=O)ccc1OC]

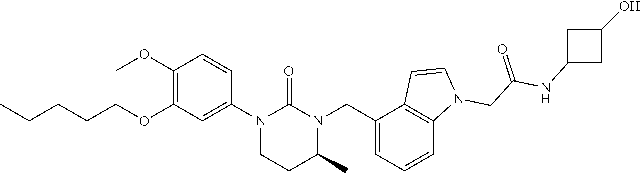 [SMILES: CCCCCOc1cc(N2CC[C@H](C)N(Cc3cccc4c3ccn4CC(=O)NC3CC(O)C3)C2=O)ccc1OC]

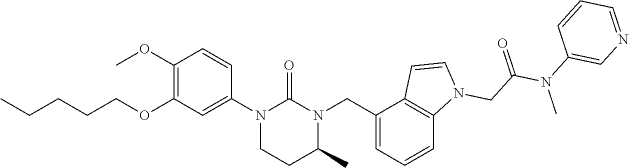 [SMILES: CCCCCOc1cc(N2CC[C@H](C)N(Cc3cccc4c3ccn4CC(=O)N(C)c3cccnc3)C2=O)ccc1OC]

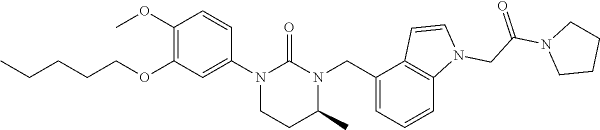 [SMILES: CCCCCOc1cc(N2CC[C@H](C)N(Cc3cccc4c3ccn4CC(=O)N3CCCC3)C2=O)ccc1OC]

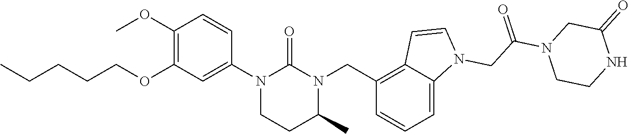 [SMILES: CCCCCOc1cc(N2CC[C@H](C)N(Cc3cccc4c3ccn4CC(=O)N3CCNC(=O)C3)C2=O)ccc1OC]

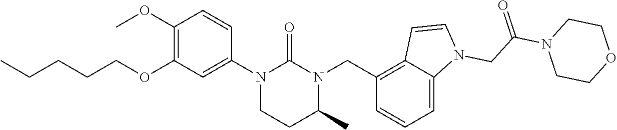 [SMILES: CCCCCOc1cc(N2CC[C@H](C)N(Cc3cccc4c3ccn4CC(=O)N3CCOCC3)C2=O)ccc1OC]

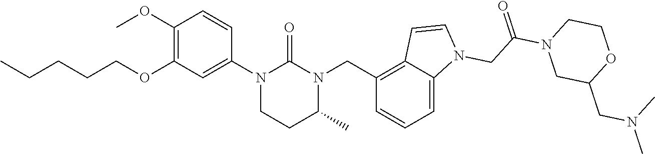 [SMILES: CCCCCOc1cc(N2CC[C@@H](C)N(Cc3cccc4c3ccn4CC(=O)N3CCOC(CN(C)C)C3)C2=O)ccc1OC]

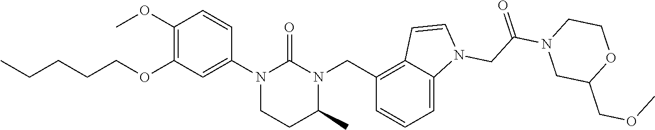 [SMILES: CCCCCOc1cc(N2CC[C@H](C)N(Cc3cccc4c3ccn4CC(=O)N3CCOC(COC)C3)C2=O)ccc1OC]

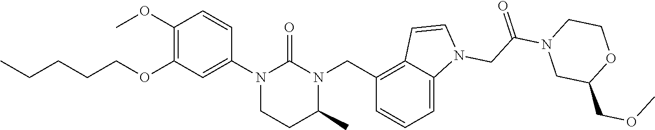 [SMILES: CCCCCOc1cc(N2CC[C@H](C)N(Cc3cccc4c3ccn4CC(=O)N3CCO[C@@H](COC)C3)C2=O)ccc1OC]

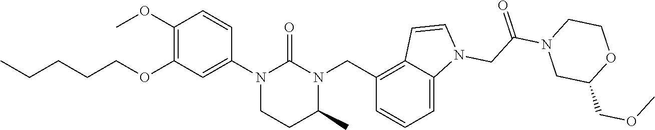 [SMILES: CCCCCOc1cc(N2CC[C@H](C)N(Cc3cccc4c3ccn4CC(=O)N3CCO[C@H](COC)C3)C2=O)ccc1OC]

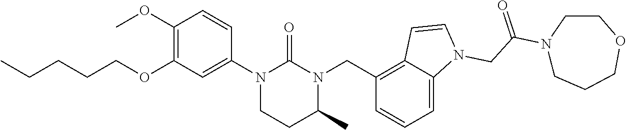 [SMILES: CCCCCOc1cc(N2CC[C@H](C)N(Cc3cccc4c3ccn4CC(=O)N3CCCOCC3)C2=O)ccc1OC]

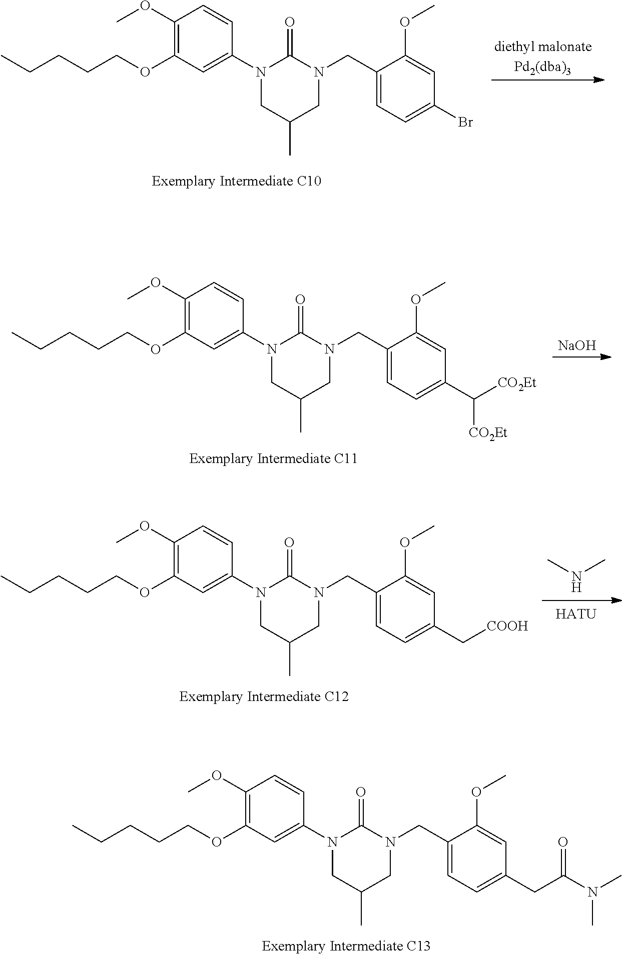

Cesium carbonate (16.9 g, 68 mmol) is added to a solution of Exemplary Intermediate C10 (1-(4-bromo-2-methoxybenzyl)-3-(4-methoxy-3-(pentyloxy)phenyl)-5-methyltetrahydro-pyrimidin-2(1H)-one; 17.0 g, 34 mmol) and diethyl malonate (10.9 g, 68 mmol) in anhydrous N,N-dimethylformamide (150 mL). The mixture is sparged with dry nitrogen for 5 min, then tris(dibenzylideneacetone)dipalladium(0) (1.0 g, 1.1 mmol) and SPhos ligand (1.0 g, 2.44 mmol) are added. The mixture is heated to 95° C. and is stirred at this temperature for 12 h. After cooling to room temperature, the mixture is quenched with water (500 mL) and extracted with ethyl acetate (3×300 mL). The combined organic phases are washed with brine (3×300 mL), dried over sodium sulfate, filtered, and concentrated in vacuo. The residue is purified by column chromatography over silica gel (hexanes/ethyl acetate: 5:1 to 3:1) to afford Exemplary Intermediate C11 (diethyl 2-(3-methoxy-4-((3-(4-methoxy-3-(pentyloxy)phenyl)-5-methyl-2-oxotetrahydropyrimidin-1(2H)-yl)methyl)phenyl)malonate).

A solution of Exemplary Intermediate C11 (14.0 g, 24 mmol) and sodium hydroxide (2.0 g, 50 mmol) in a mixture of ethanol and water (1:2 v/v, 300 mL) is refluxed for 12 h. After cooling to room temperature, the mixture is washed with ethyl acetate/hexane mixture (1:1 v/v, 3×200 mL) and these washes are discarded. The remaining aqueous layer is acidified to pH of 3 with 1N hydrochloric acid then refluxed for 2 h. After cooling to room temperature, the mixture is extracted with ethyl acetate (3×300 mL). The combined organic phases are washed with brine (3×200 mL), then dried over sodium sulfate, filtered, and concentrated to afford Exemplary Intermediate C12 (2-(3-methoxy-4-((3-(4-methoxy-3-(pentyloxy)phenyl)-2-oxotetrahydro-pyrimidin-1(2H)-yl)methyl)phenyl)acetic acid).

Exemplary Intermediate C12 is dissolved in N,N-dimethylformamide, then HATU, dimethylamine, and N,N-diisopropylethylamine are added. Reaction mixture is stirred at room temperature for 2 h. Water is added to reaction mixture, and it is extracted three times with ethyl acetate. The organic layers are dried over a phase separator and concentrated in vacuo. The residue is purified by column chromatography over silica (dichloromethane/methanol: 100/0 to 97/3) to afford Exemplary Intermediate C13 (2-(3-methoxy-4-((3-(4-methoxy-3-(pentyloxy) phenyl)-5-methyl-2-oxotetrahydropyrimidin-1(2H)-yl)methyl)phenyl)-N,N-dimethylacetamide).

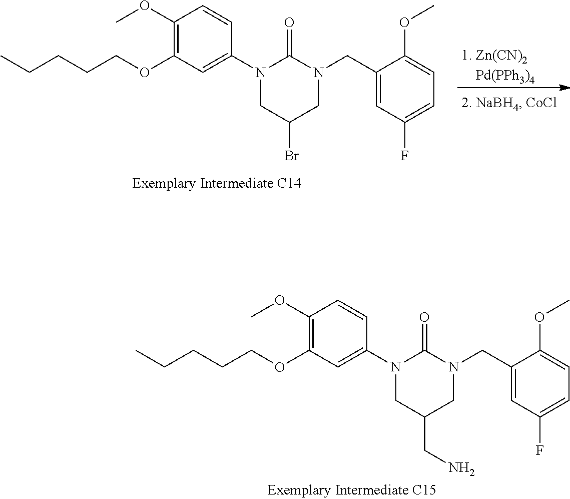

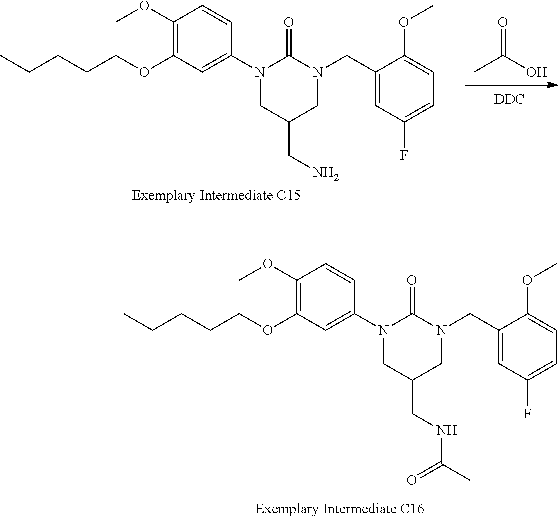

A mixture of Exemplary Intermediate C14 (5-bromo-1-(5-fluoro-2-methoxybenzyl)-3-(4-methoxy-3-(pentyloxy)phenyl)tetrahydropyrimidin-2(1H)-one), activated zinc dust, and fresh prepared tetrakis(triphenylphosphine)palladium(0) in dry N,N-dimethylformamide (350 mL) is stirred under heat and a nitrogen atmosphere overnight. The reaction mixture is cooled to room temperature, quenched with water, and extracted with ethyl acetate. The combined organic phases are washed with brine, dried over anhydrous sodium sulfate, filtered and concentrated in vacuo. The residue is suspended in hexane-ethyl acetate (10:1 v/v, 200 mL) and filtered. The collected solids are dried under vacuum to afford Exemplary Intermediate C15 (5-(aminomethyl)-1-(5-fluoro-2-methoxybenzyl)-3-(4-methoxy-3-(pentyloxy)phenyl)tetrahydropyrimidin-2(1H)-one).

Exemplary Intermediate C15 is combined with dicyclohexylcarbodiimide (DCC) and acetic acid to afford Exemplary Intermediate C16 (N-((1-(5-fluoro-2-methoxybenzyl)-3-(4-methoxy-3-(pentyloxy)phenyl)-2-oxohexahydropyrimidin-5-yl)methyl)acetamide).

The following compounds are prepared in a similar manner as Exemplary Intermediate C16, as described above.

| Bromide | Acid |
|---|---|
| 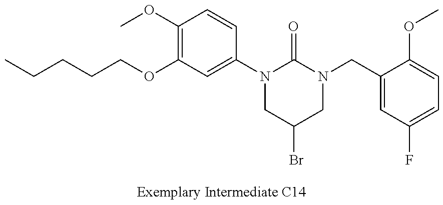 [SMILES: CCCCCOc1cc(N2CC(Br)CN(Cc3cc(F)ccc3OC)C2=O)ccc1OC] | 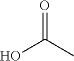 |
| 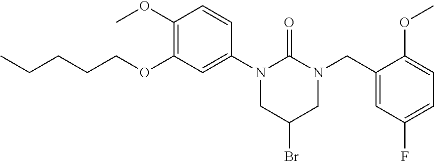 [SMILES: CCCCCOc1cc(N2CC(Br)CN(Cc3cc(F)ccc3OC)C2=O)ccc1OC] | 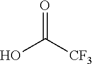 |
| 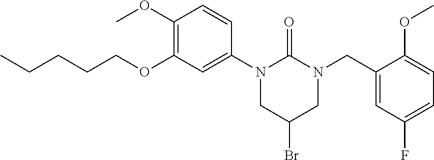 [SMILES: CCCCCOc1cc(N2CC(Br)CN(Cc3cc(F)ccc3OC)C2=O)ccc1OC] | 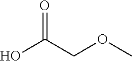 |
| 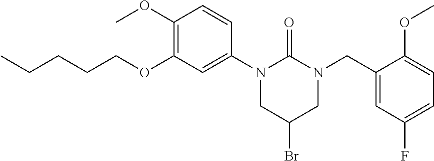 [SMILES: CCCCCOc1cc(N2CC(Br)CN(Cc3cc(F)ccc3OC)C2=O)ccc1OC] | 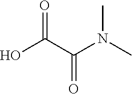 |
| 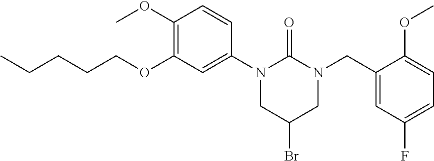 [SMILES: CCCCCOc1cc(N2CC(Br)CN(Cc3cc(F)ccc3OC)C2=O)ccc1OC] | 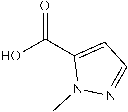 |
| 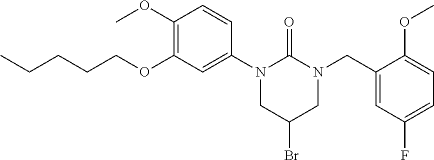 [SMILES: CCCCCOc1cc(N2CC(Br)CN(Cc3cc(F)ccc3OC)C2=O)ccc1OC] | 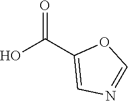 |
| 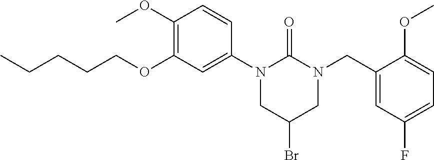 [SMILES: CCCCCOc1cc(N2CC(Br)CN(Cc3cc(F)ccc3OC)C2=O)ccc1OC] | 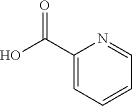 |

Compound

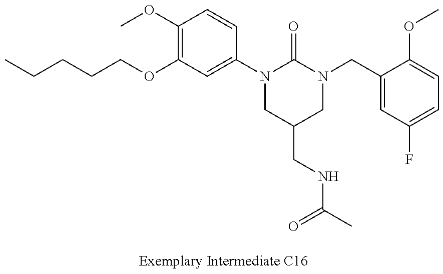 [SMILES: CCCCCOc1cc(N2CC(CNC(C)=O)CN(Cc3cc(F)ccc3OC)C2=O)ccc1OC]

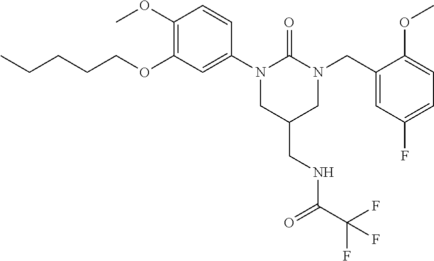 [SMILES: CCCCCOc1cc(N2CC(CNC(=O)C(F)(F)F)CN(Cc3cc(F)ccc3OC)C2=O)ccc1OC]

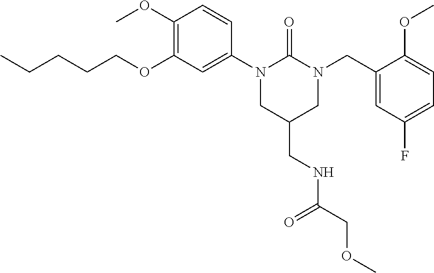 [SMILES: CCCCCOc1cc(N2CC(CNC(=O)COC)CN(Cc3cc(F)ccc3OC)C2=O)ccc1OC]

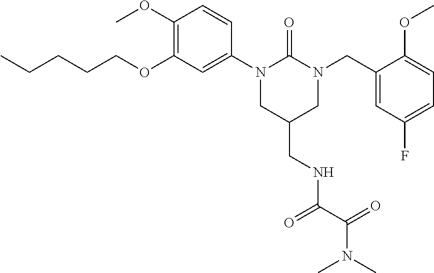 [SMILES: CCCCCOc1cc(N2CC(CNC(=O)C(=O)N(C)C)CN(Cc3cc(F)ccc3OC)C2=O)ccc1OC]

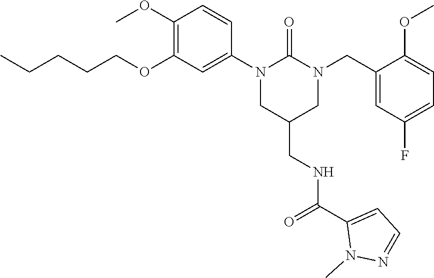 [SMILES: CCCCCOc1cc(N2CC(CNC(=O)c3ccnn3C)CN(Cc3cc(F)ccc3OC)C2=O)ccc1OC]

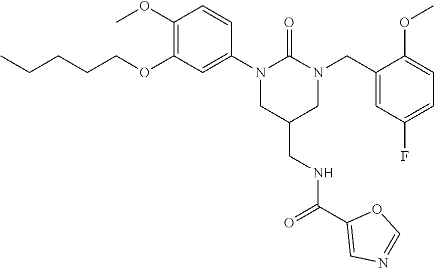 [SMILES: CCCCCOc1cc(N2CC(CNC(=O)c3cnco3)CN(Cc3cc(F)ccc3OC)C2=O)ccc1OC]

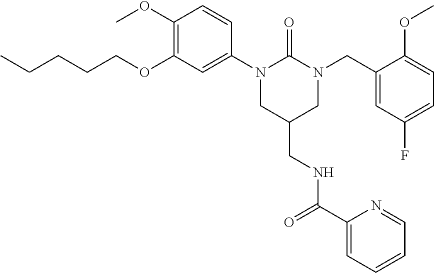 [SMILES: CCCCCOc1cc(N2CC(CNC(=O)c3ccccn3)CN(Cc3cc(F)ccc3OC)C2=O)ccc1OC]

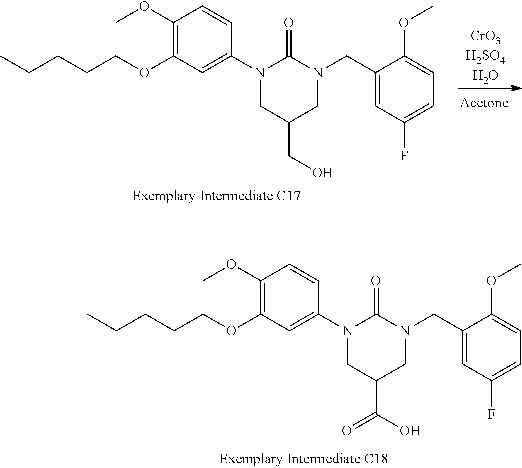

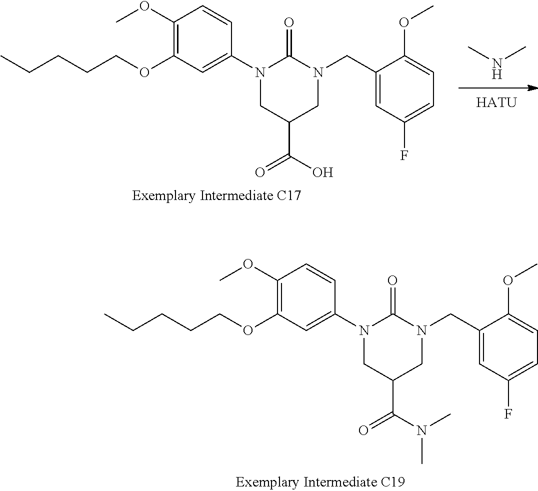

Into a mixture of Exemplary Intermediate C17 (1-(5-fluoro-2-methoxybenzyl)-5-(hydroxymethyl)-3-(4-methoxy-3-(pentyloxy)phenyl)tetrahydropyrimidin-2(1H)-one) in acetone is added a solution of chromium trioxide in diluted sulfuric acid. The reaction affords Exemplary Intermediate C18 (1-(5-fluoro-2-methoxybenzyl)-3-(4-methoxy-3-(pentyloxy)phenyl)-2-oxohexahydropyrimidine-5-carboxylic acid).

Exemplary Intermediate C18 is dissolved in N,N-dimethylformamide, then HATU, dimethylamine, and N,N-diisopropylethylamine are added. Reaction mixture is stirred at room temperature for 2 h. Water is added to reaction mixture, and it is extracted three times with ethyl acetate. The organic layers are dried over a phase separator and concentrated in vacuo. The residue is purified by column chromatography over silica to afford Exemplary Intermediate C19 (1-(5-fluoro-2-methoxybenzyl)-3-(4-methoxy-3-(pentyloxy)phenyl)-N,N-dimethyl-2-oxohexahydro-pyrimidine-5-carboxamide).

The following compounds are prepared in a similar manner as Exemplary Intermediate C19, as described above.

| Alcohol | Amine |
|---|---|
| 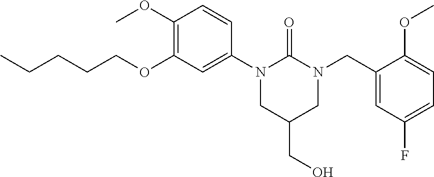 [SMILES: CCCCCOc1cc(N2CC(CO)CN(Cc3cc(F)ccc3OC)C2=O)ccc1OC] | NH₃ |
| 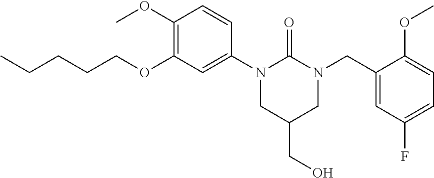 [SMILES: CCCCCOc1cc(N2CC(CO)CN(Cc3cc(F)ccc3OC)C2=O)ccc1OC] | MeNH₂ |
| 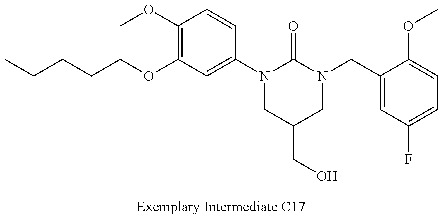 [SMILES: CCCCCOc1cc(N2CC(CO)CN(Cc3cc(F)ccc3OC)C2=O)ccc1OC] | 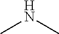 |
| 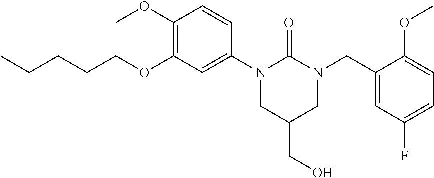 [SMILES: CCCCCOc1cc(N2CC(CO)CN(Cc3cc(F)ccc3OC)C2=O)ccc1OC] | 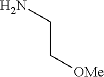 |
| 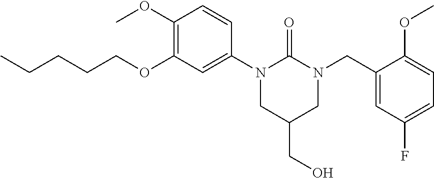 [SMILES: CCCCCOc1cc(N2CC(CO)CN(Cc3cc(F)ccc3OC)C2=O)ccc1OC] | 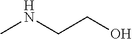 |
| 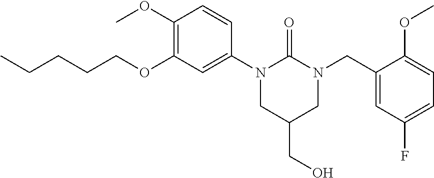 [SMILES: CCCCCOc1cc(N2CC(CO)CN(Cc3cc(F)ccc3OC)C2=O)ccc1OC] | 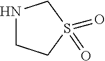 |
| 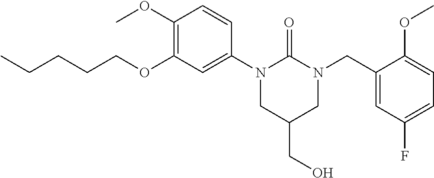 [SMILES: CCCCCOc1cc(N2CC(CO)CN(Cc3cc(F)ccc3OC)C2=O)ccc1OC] | 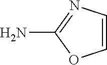 |
| 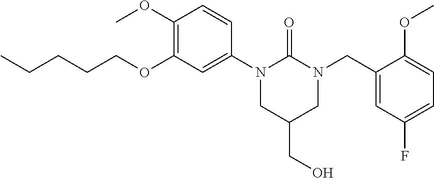 [SMILES: CCCCCOc1cc(N2CC(CO)CN(Cc3cc(F)ccc3OC)C2=O)ccc1OC] | 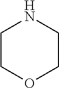 |

Compound

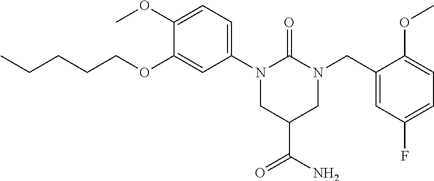 [SMILES: CCCCCOc1cc(N2CC(C(N)=O)CN(Cc3cc(F)ccc3OC)C2=O)ccc1OC]

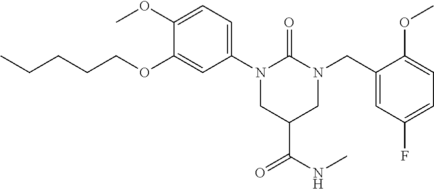 [SMILES: CCCCCOc1cc(N2CC(C(=O)NC)CN(Cc3cc(F)ccc3OC)C2=O)ccc1OC]

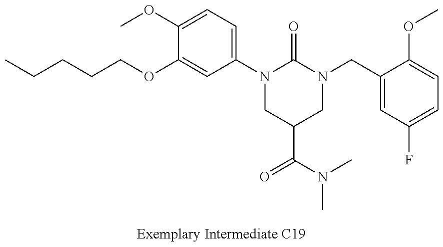 [SMILES: CCCCCOc1cc(N2CC(C(=O)N(C)C)CN(Cc3cc(F)ccc3OC)C2=O)ccc1OC]

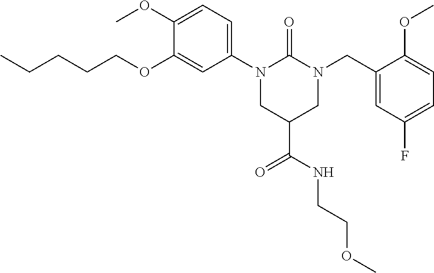 [SMILES: CCCCCOc1cc(N2CC(C(=O)NCCOC)CN(Cc3cc(F)ccc3OC)C2=O)ccc1OC]

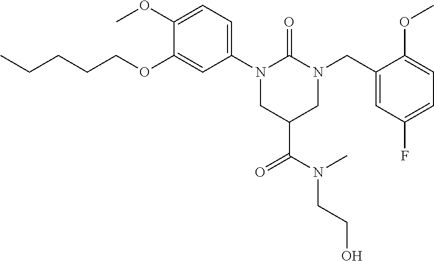 [SMILES: CCCCCOc1cc(N2CC(C(=O)N(C)CCO)CN(Cc3cc(F)ccc3OC)C2=O)ccc1OC]

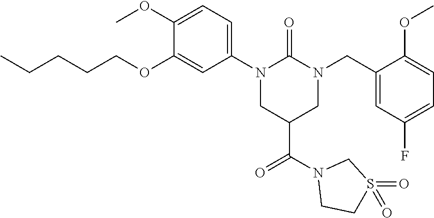 [SMILES: CCCCCOc1cc(N2CC(C(=O)N3CCS(=O)(=O)C3)CN(Cc3cc(F)ccc3OC)C2=O)ccc1OC]

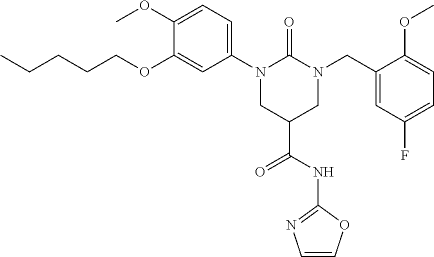 [SMILES: CCCCCOc1cc(N2CC(C(=O)Nc3ncco3)CN(Cc3cc(F)ccc3OC)C2=O)ccc1OC]

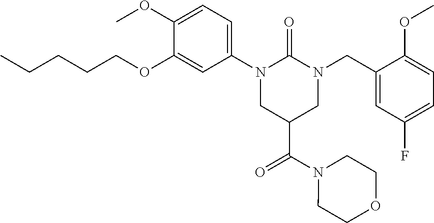 [SMILES: CCCCCOc1cc(N2CC(C(=O)N3CCOCC3)CN(Cc3cc(F)ccc3OC)C2=O)ccc1OC]

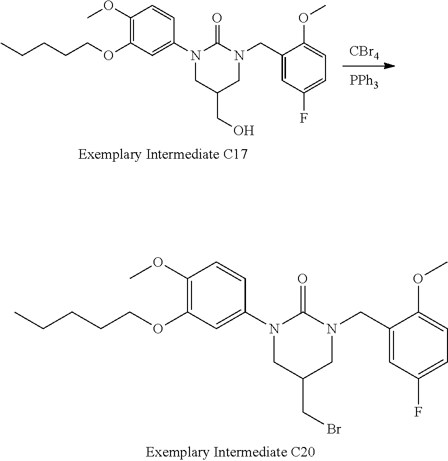

Exemplary Intermediate C17 (1-(5-fluoro-2-methoxybenzyl)-5-(hydroxymethyl)-3-(4-methoxy-3-(pentyloxy)phenyl)tetrahydropyrimidin-2(1H)-one) is combined with carbon tetrabromide and triphenylphosphine to afford Exemplary Intermediate C20 (5-(bromomethyl)-1-(5-fluoro-2-methoxybenzyl)-3-(4-methoxy-3-(pentyloxy)phenyl)tetrahydropyrimidin-2(1H)-one).

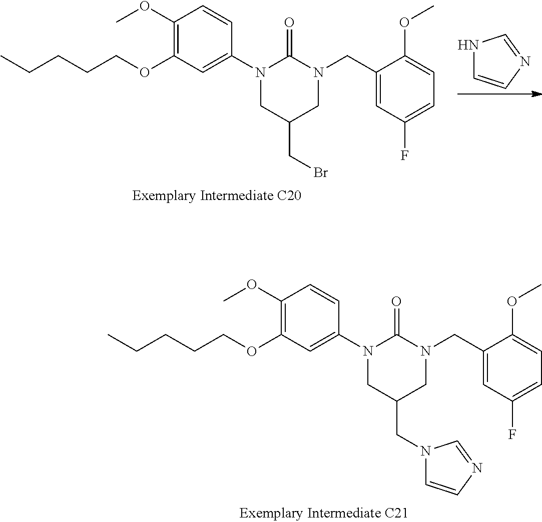

Exemplary Intermediate C20 is reacted with 1H-imidazole and sodium hydride. The resulting reaction affords Exemplary Intermediate C21 (5-((1H-imidazol-1-yl)methyl)-1-(5-fluoro-2-methoxybenzyl)-3-(4-methoxy-3-(pentyloxy)phenyl)tetrahydropyrimidin-2(1H)-one).

The following compounds are prepared in a similar manner as Exemplary Intermediate C21, as described above.

| Bromide | Amine |
|---|---|
| 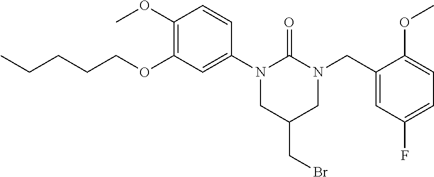 [SMILES: CCCCCOc1cc(N2CC(CBr)CN(Cc3cc(F)ccc3OC)C2=O)ccc1OC] | 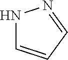 |
| 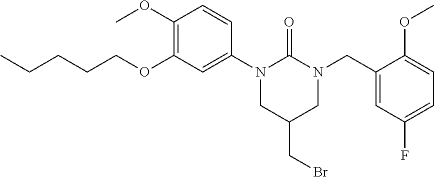 [SMILES: CCCCCOc1cc(N2CC(CBr)CN(Cc3cc(F)ccc3OC)C2=O)ccc1OC] | 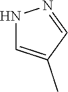 |
| 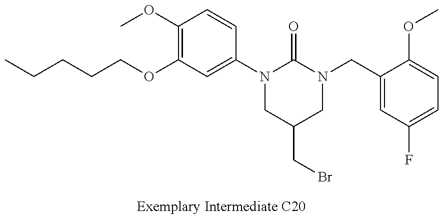 [SMILES: CCCCCOc1cc(N2CC(CBr)CN(Cc3cc(F)ccc3OC)C2=O)ccc1OC] | 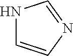 |
| 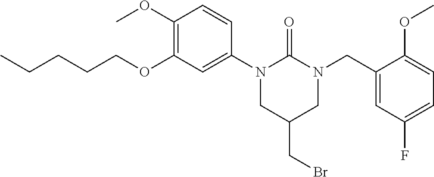 [SMILES: CCCCCOc1cc(N2CC(CBr)CN(Cc3cc(F)ccc3OC)C2=O)ccc1OC] | 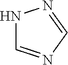 |
| 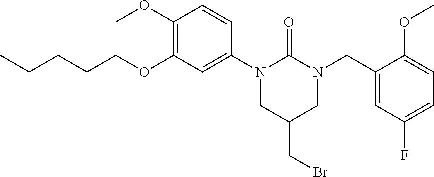 [SMILES: CCCCCOc1cc(N2CC(CBr)CN(Cc3cc(F)ccc3OC)C2=O)ccc1OC] | 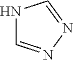 |
| 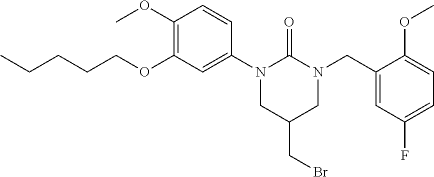 [SMILES: CCCCCOc1cc(N2CC(CBr)CN(Cc3cc(F)ccc3OC)C2=O)ccc1OC] | 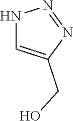 |
| 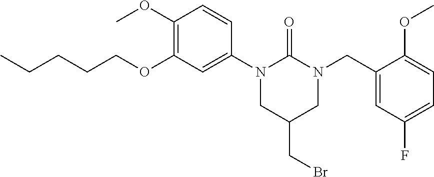 [SMILES: CCCCCOc1cc(N2CC(CBr)CN(Cc3cc(F)ccc3OC)C2=O)ccc1OC] | 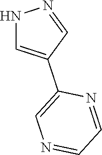 [SMILES: c1cnc(-c2cn[nH]c2)cn1] |
| 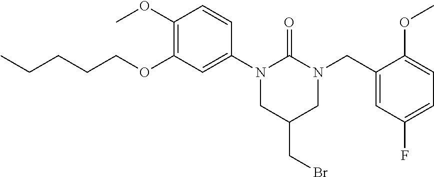 [SMILES: CCCCCOc1cc(N2CC(CBr)CN(Cc3cc(F)ccc3OC)C2=O)ccc1OC] | 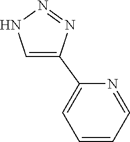 [SMILES: c1ccc(-c2c[nH]nn2)nc1] |
| 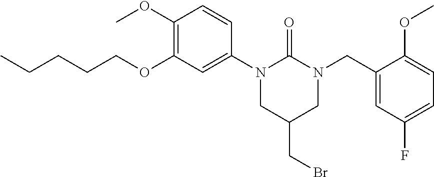 [SMILES: CCCCCOc1cc(N2CC(CBr)CN(Cc3cc(F)ccc3OC)C2=O)ccc1OC] | 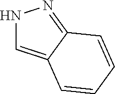 |
| 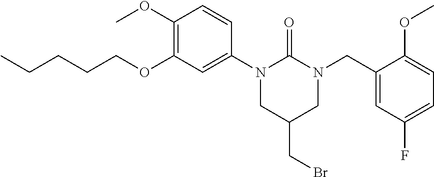 [SMILES: CCCCCOc1cc(N2CC(CBr)CN(Cc3cc(F)ccc3OC)C2=O)ccc1OC] | 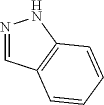 |

Compound

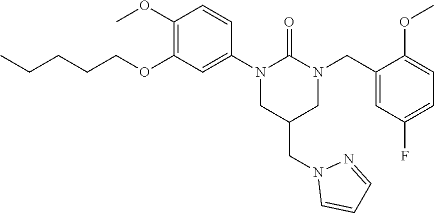 [SMILES: CCCCCOc1cc(N2CC(Cn3cccn3)CN(Cc3cc(F)ccc3OC)C2=O)ccc1OC]

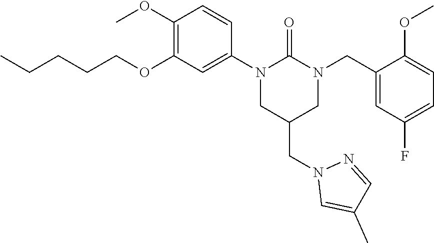 [SMILES: CCCCCOc1cc(N2CC(Cn3cc(C)cn3)CN(Cc3cc(F)ccc3OC)C2=O)ccc1OC]

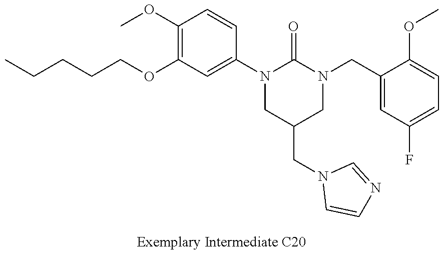 [SMILES: CCCCCOc1cc(N2CC(Cn3ccnc3)CN(Cc3cc(F)ccc3OC)C2=O)ccc1OC]

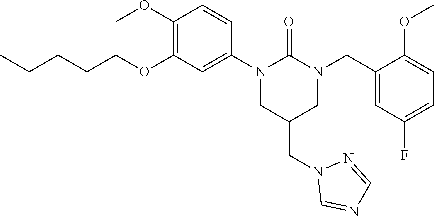 [SMILES: CCCCCOc1cc(N2CC(Cn3cncn3)CN(Cc3cc(F)ccc3OC)C2=O)ccc1OC]

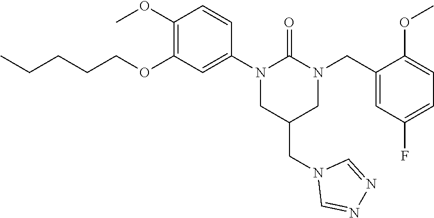 [SMILES: CCCCCOc1cc(N2CC(Cn3cnnc3)CN(Cc3cc(F)ccc3OC)C2=O)ccc1OC]

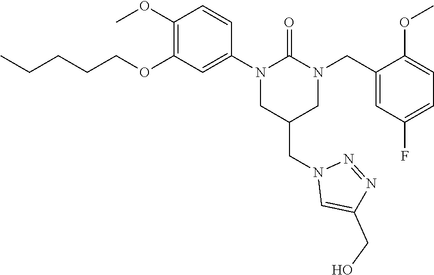 [SMILES: CCCCCOc1cc(N2CC(Cn3cc(CO)nn3)CN(Cc3cc(F)ccc3OC)C2=O)ccc1OC]

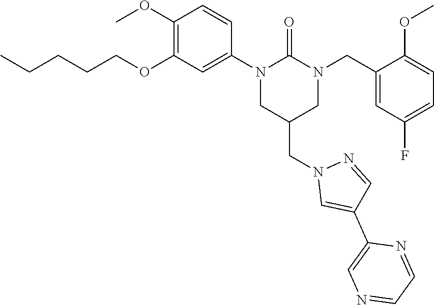 [SMILES: CCCCCOc1cc(N2CC(Cn3cc(-c4cnccn4)cn3)CN(Cc3cc(F)ccc3OC)C2=O)ccc1OC]

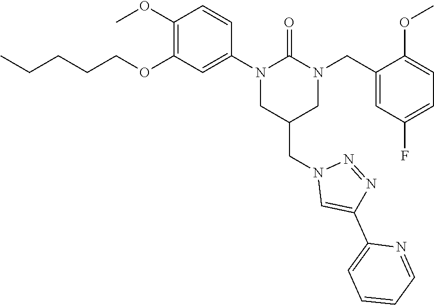 [SMILES: CCCCCOc1cc(N2CC(Cn3cc(-c4ccccn4)nn3)CN(Cc3cc(F)ccc3OC)C2=O)ccc1OC]

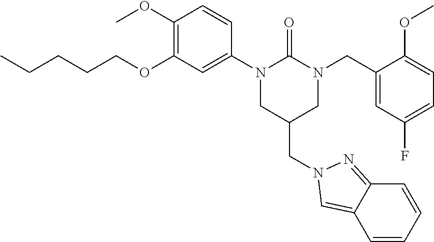 [SMILES: CCCCCOc1cc(N2CC(Cn3cc4ccccc4n3)CN(Cc3cc(F)ccc3OC)C2=O)ccc1OC]

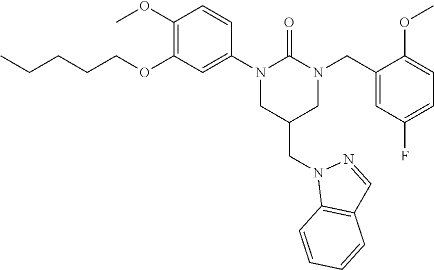 [SMILES: CCCCCOc1cc(N2CC(Cn3ncc4ccccc43)CN(Cc3cc(F)ccc3OC)C2=O)ccc1OC]

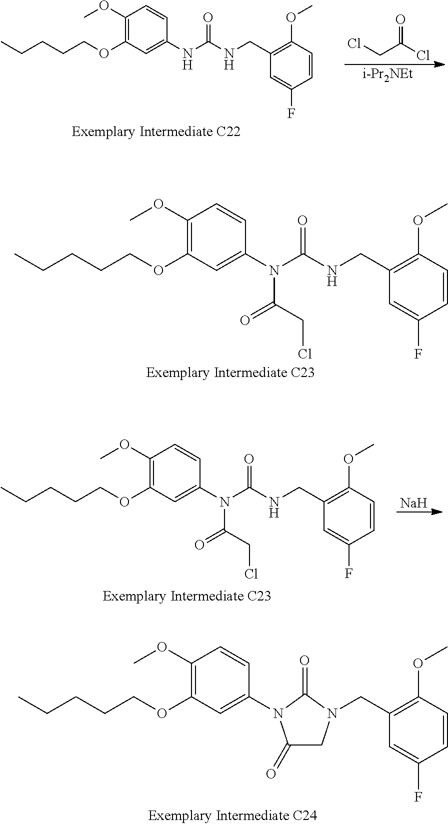

To a solution of Exemplary Intermediate C22 (1-(5-fluoro-2-methoxybenzyl)-3-(4-methoxy-3-(pentyloxy)phenyl)urea) in dry tetrahydrofuran is added N,N-diisopropylethylamine and 2-chloroacetyl chloride in a dropwise fashion. The reaction mixture is stirred for 3 h at room temperature. An additional 1.0 eq of N,N-diisopropylethylamine and 1.5 eq of 2-chloroacetyl chloride are added, and the reaction is stirred another 3 h. 1.5 eq of N,N-diisopropylethylamine and 2.0 eq of 2-chloroacetyl chloride are added, and the reaction is stirred for another 2 h. 1.0 eq of diisopropylethylamine and 1.5 eq of 2-chloroacetyl chloride are added, and the reaction is stirred for another 1 h. The solvent is evaporated in vacuo, and the residue is taken up in water then extracted with ethyl acetate. The combined organic layers are washed with brine, dried over sodium sulfate, and evaporated in vacuo to afford Exemplary Intermediate C23 (2-chloro-N-((5-fluoro-2-methoxybenzyl)carbamoyl)-N-(4-methoxy-3-(pentyloxy)phenyl)acetamide).

To a stirred solution of Exemplary Intermediate C23 in dry N,N-dimethylformamide is added sodium hydride. The reaction is stirred for 3 h at room temperature. Another 0.5 eq of sodium hydride is added, and the reaction temperature increased to 50° C. The reaction is stirred at this temperature another 2 h. After cooling to room temperature, the reaction is diluted with water and extracted twice with ethyl acetate. The combined organic layers are washed with brine, dried over sodium sulfate, and concentrated in vacuo to afford Exemplary Intermediate C24 (1-(5-fluoro-2-methoxybenzyl)-3-(4-methoxy-3-(pentyloxy)phenyl)imidazolidine-2,4-dione).

The following compounds are prepared in a similar manner as Exemplary Intermediate C24, as described above.

| | Acyl |
|---|---|
| Urea | Chloride |
| 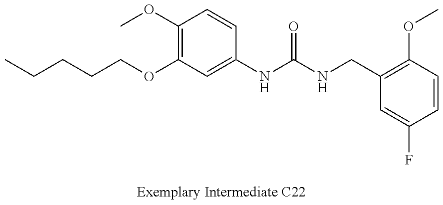 [SMILES: CCCCCOc1cc(NC(=O)NCc2cc(F)ccc2OC)ccc1OC] | 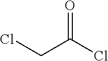 |
| 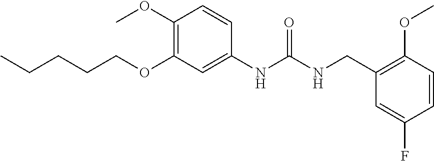 [SMILES: CCCCCOc1cc(NC(=O)NCc2cc(F)ccc2OC)ccc1OC] | 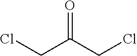 |
| 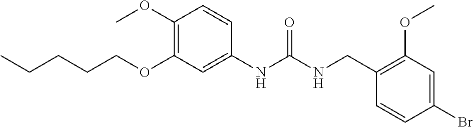 [SMILES: CCCCCOc1cc(NC(=O)NCc2ccc(Br)cc2OC)ccc1OC] | 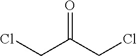 |

Compound

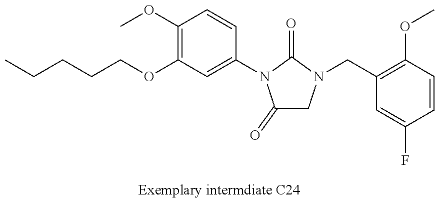 [SMILES: CCCCCOc1cc(N2C(=O)CN(Cc3cc(F)ccc3OC)C2=O)ccc1OC]

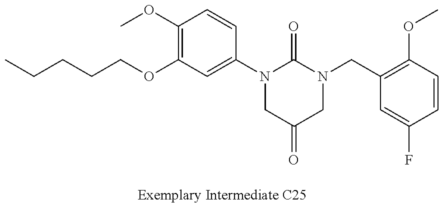 [SMILES: CCCCCOc1cc(N2CC(=O)CN(Cc3cc(F)ccc3OC)C2=O)ccc1OC]

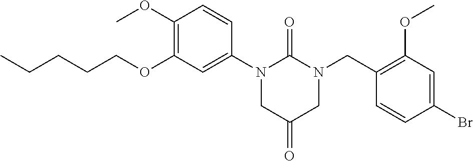 [SMILES: CCCCCOc1cc(N2CC(=O)CN(Cc3ccc(Br)cc3OC)C2=O)ccc1OC]

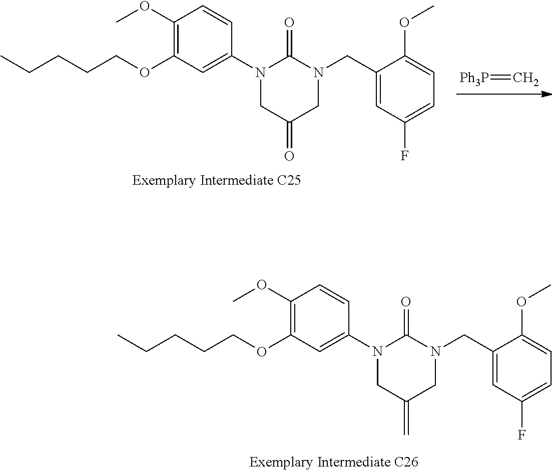

To a solution of Exemplary Intermediate C25 (1-(5-fluoro-2-methoxybenzyl)-3-(4-methoxy-3-(pentyloxy)phenyl)tetrahydropyrimidine-2,5-dione) in tetrahydrofuran is added phosphonium ylide. The reaction mixture is stirred for under heat to afford Exemplary Intermediate C26 (1-(5-fluoro-2-methoxybenzyl)-3-(4-methoxy-3-(pentyloxy)phenyl)-5-methylenetetrahydropyrimidin-2(1H)-one).

The following compounds are prepared in a similar manner as Exemplary Intermediate C26, as described above.

| Ketone | Alkene |
|---|---|
| 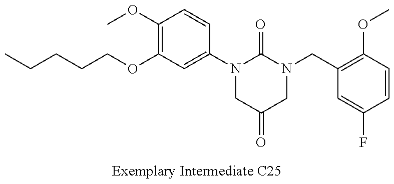 [SMILES: CCCCCOc1cc(N2CC(=O)CN(Cc3cc(F)ccc3OC)C2=O)ccc1OC] | 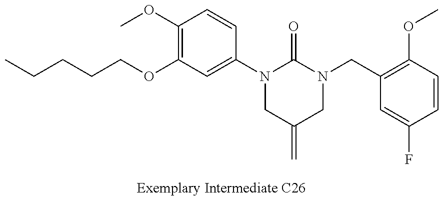 [SMILES: C=C1CN(Cc2cc(F)ccc2OC)C(=O)N(c2ccc(OC)c(OCCCCC)c2)C1] |
| 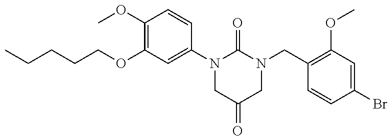 [SMILES: CCCCCOc1cc(N2CC(=O)CN(Cc3ccc(Br)cc3OC)C2=O)ccc1OC] | 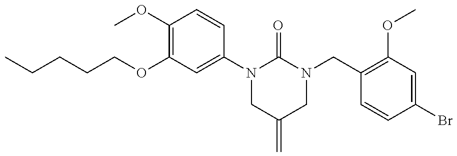 [SMILES: C=C1CN(Cc2ccc(Br)cc2OC)C(=O)N(c2ccc(OC)c(OCCCCC)c2)C1] |

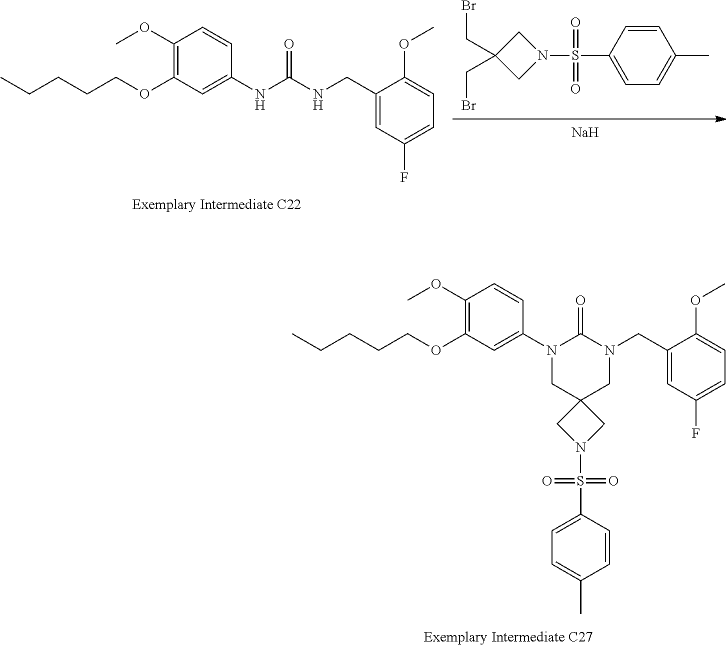

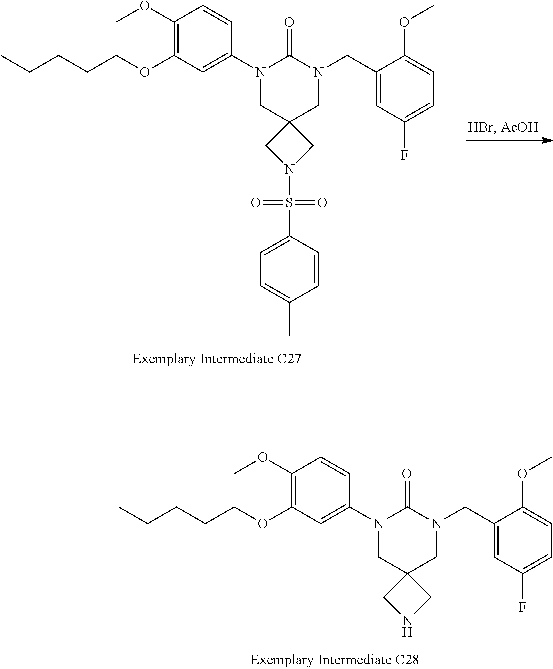

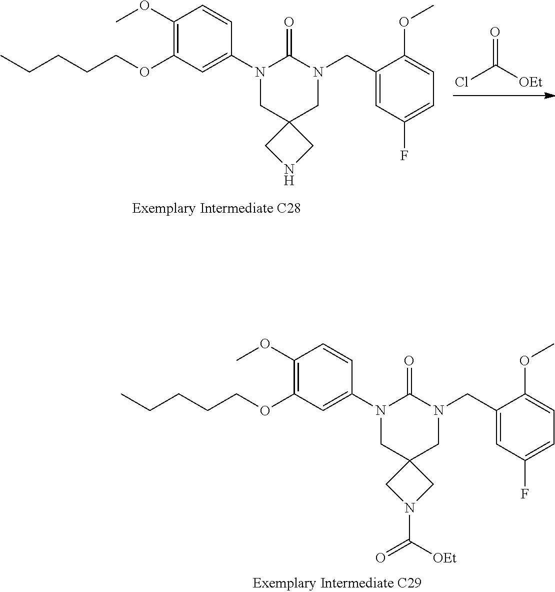

Exemplary Intermediate C22 (1-(5-fluoro-2-methoxybenzyl)-3-(4-methoxy-3-(pentyloxy)phenyl)urea) is dissolved in tetrahydrofuran and treated with sodium hydride, then 3,3-bis(bromomethyl)-1-tosylazetidine. Reaction mixture is stirred at 50° C. for 3 hrs. More sodium hydride and 3,3-bis(bromomethyl)-1-tosylazetidine is added, then the mixture is stirred at 50° C. overnight. Water is added to the mixture and it is extracted three times with ethyl acetate. The combined organic layers are dried over sodium sulfate, filtered, and concentrated in vacuo to afford Exemplary Intermediate C27 (6-(5-fluoro-2-methoxy benzyl)-8-(4-methoxy-3-(pentyloxy)phenyl)-2-tosyl-2,6,8-triazaspiro[3.5]nonan-7-one).

Exemplary Intermediate C27 is mixed with hydrogen bromide and acetic acid and reacted at 70° C. to afford Exemplary Intermediate 28 (6-(5-fluoro-2-methoxybenzyl)-8-(4-methoxy-3-(pentyloxy)phenyl)-2,6,8-triazaspiro[3.5]nonan-7-one).

Exemplary Intermediate C28 is mixed with tetra-n-butylammonium hydrogen sulfate and potassium carbonate, follow by the addition of ethyl chloroformate. The mixture is reacted to afford Exemplary Intermediate C29 (ethyl 6-(5-fluoro-2-methoxybenzyl)-8-(4-methoxy-3-(pentyloxy)phenyl)-7-oxo-2,6,8-triazaspiro[3.5]nonane-2-carboxylate).

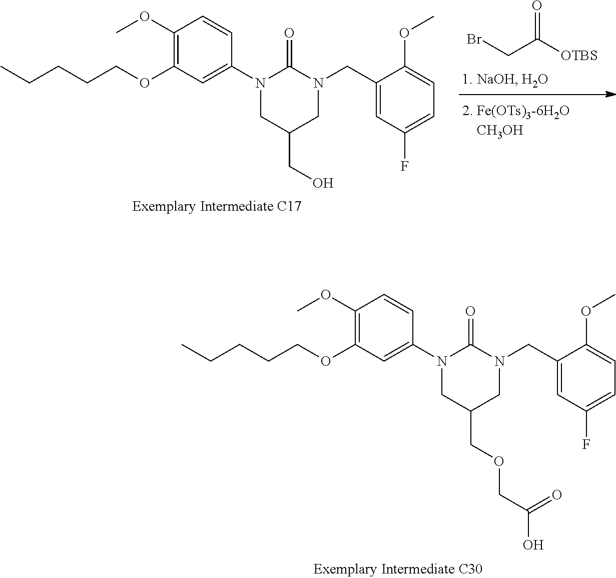

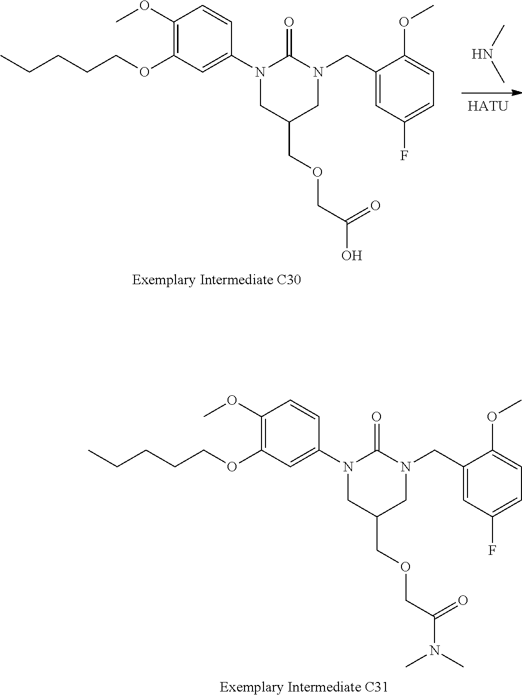

Exemplary Intermediate C17 (1-(5-fluoro-2-methoxybenzyl)-5-(hydroxymethyl)-3-(4-methoxy-3-(pentyloxy)phenyl)tetrahydropyrimidin-2(1H)-one) is combined with sodium hydroxide in water, followed by the addition tert-Butyldimethylsilyl bromoacetate. The mixture is refluxed for at least 3 hours under heat. Solids are filtered and concentrated in vacuo, and then dissolved into methanol. Iron (III) p-toluenesulfonate hexahydrate (2.0 mol %) is added, and the mixture is refluxed at room temperature. The resulting acid product is filtered and concentrated in vacuo, then purified by column chromatography to afford Exemplary Intermediate C30 (1-(5-fluoro-2-methoxybenzyl)-3-(4-methoxy-3-(pentyloxy)phenyl)-2-oxohexahydropyrimidine-5-carboxylic acid).

Exemplary Intermediate C30 is dissolved in N,N-dimethylformamide, then HATU, dimethylamine, and N,N-diisopropylethylamine are added. Reaction mixture is stirred at room temperature for 2 h. Water is added to reaction mixture, and it is extracted three times with ethyl acetate. The organic layers are dried over a phase separator and concentrated in vacuo. The residue is purified by column chromatography over silica to afford Exemplary Intermediate C31 (2-((1-(5-fluoro-2-methoxybenzyl)-3-(4-methoxy-3-(pentyloxy)phenyl)-2-oxohexahydropyrimidin-5-yl)methoxy)-N,N-dimethylacetamide).

The following compounds are prepared in a similar manner as Exemplary Intermediate C31, as described above.

| Alcohol | Amine |
|---|---|
| 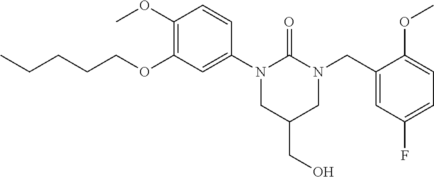 [SMILES: CCCCCOc1cc(N2CC(CO)CN(Cc3cc(F)ccc3OC)C2=O)ccc1OC] | NH₃ |
| 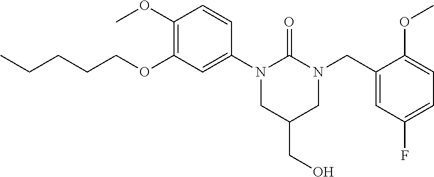 [SMILES: CCCCCOc1cc(N2CC(CO)CN(Cc3cc(F)ccc3OC)C2=O)ccc1OC] | H₂N—CH₃ |
| 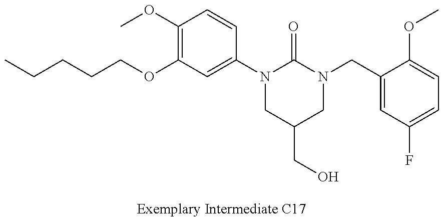 [SMILES: CCCCCOc1cc(N2CC(CO)CN(Cc3cc(F)ccc3OC)C2=O)ccc1OC] | 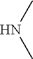 |
| 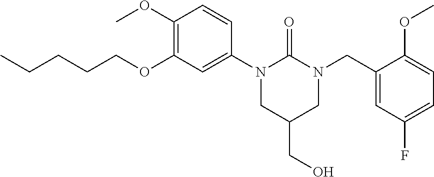 [SMILES: CCCCCOc1cc(N2CC(CO)CN(Cc3cc(F)ccc3OC)C2=O)ccc1OC] | 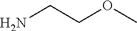 |
| 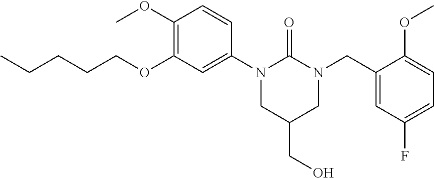 [SMILES: CCCCCOc1cc(N2CC(CO)CN(Cc3cc(F)ccc3OC)C2=O)ccc1OC] | 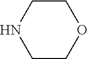 |

Compound

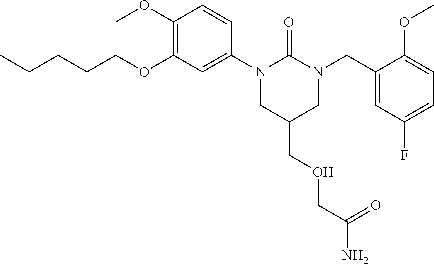 [SMILES: CCCCCOc1cc(N2CC(COCC(N)=O)CN(Cc3cc(F)ccc3OC)C2=O)ccc1OC]

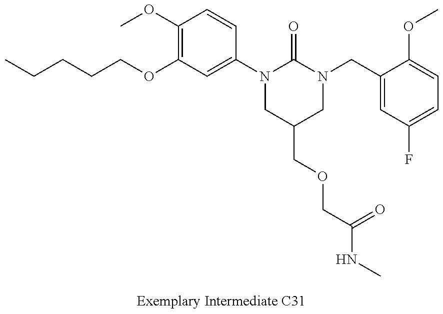 [SMILES: CCCCCOc1cc(N2CC(COCC(=O)NC)CN(Cc3cc(F)ccc3OC)C2=O)ccc1OC]

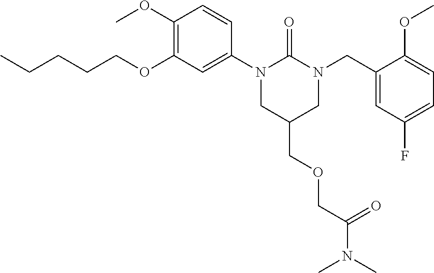 [SMILES: CCCCCOc1cc(N2CC(COCC(=O)N(C)C)CN(Cc3cc(F)ccc3OC)C2=O)ccc1OC]

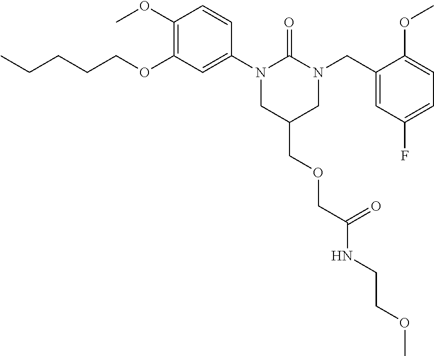 [SMILES: CCCCCOc1cc(N2CC(COCC(=O)NCCOC)CN(Cc3cc(F)ccc3OC)C2=O)ccc1OC]

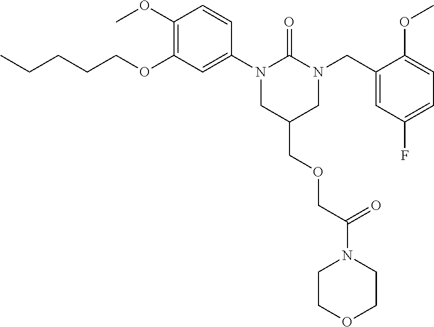 [SMILES: CCCCCOc1cc(N2CC(COCC(=O)N3CCOCC3)CN(Cc3cc(F)ccc3OC)C2=O)ccc1OC]

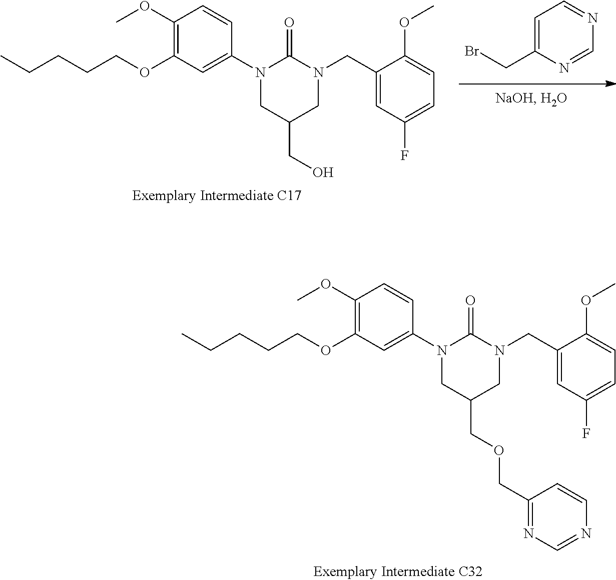

Exemplary Intermediate C17 (1-(5-fluoro-2-methoxybenzyl)-5-(hydroxymethyl)-3-(4-methoxy-3-(pentyloxy)phenyl)tetrahydropyrimidin-2(1H)-one) is combined with sodium hydroxide in water, followed by the addition 4-(bromomethyl)pyrimidine. The mixture is refluxed for at least 3 hours under heat. Solids are filtered and concentrated in vacuo, then purified by column chromatography to afford Exemplary Intermediate C32 (1-(5-fluoro-2-methoxybenzyl)-3-(4-methoxy-3-(pentyloxy)phenyl)-5-((pyrimidin-4-ylmethoxy)methyl) tetrahydropyrimidin-2(1H)-one).

### Example 27. Compound Analysis by Surface Plasmon Resonance (SPR)

C5 inhibitor candidate compounds were synthesized according to standard methods known in the art [see, e.g. Morrison and Boyd in “Organic Chemistry”, 6^{th} edition, Prentice Hall (1992)], and analyzed using surface plasmon resonance (SPR) technology to generate data on the affinity, specificity, and kinetics of compound interactions with human C5 complement protein in real time without the need for labeling.

SensiQ FE SPR system (SensiQ Technologies, Oklahoma City, Okla.) was used to provide sensitive and accurate detection of binding of small molecules to the very large C5 protein (MW=195,000 Da). The chip was prepared by preconditioning the sensor according to the protocol of the SensiQ FE using 10 mM HCl, 50 mM NAOH and 0.1% SDS. The sensor chip was activated by using a mixture of fresh EDAC (1-ethyl-3-(-3-dimethylaminopropyl) carbodiimide) (Sigma Co., St. Louis, Mo.) and NHS (N-hydroxy succinimide) (Sigma Co., St. Louis, Mo.). Human C5 was surface immobilized to a Pioneer Biosensor chip via random amine coupling (>12,000 RU) which makes use of the N-terminus and ε-amino groups of lysine residues of the protein ligand. Immobilization was done by injecting 30-40 ug/ml C5 in 10 mM NaAc pH 4.5 onto designated channels at a rate of 10 μL/minute for 12 minutes, targeting RL>12000 RU for small molecules.

Compounds were diluted in DMSO in a format of 100-fold final concentration and 3-fold serial dilution (5 or 6 dilutions). The 100-fold compounds were transferred to one fold DMSO-free assay buffer in the 96-well test plate. The compound solution was injected at a rate of 60 μL/minute for 30-60 seconds, followed by 60-90 seconds dissociation time, buffer flushing and/or priming. Blank solution (1% DMSO assay buffer) was run for every 6 injections of compounds. Double reference by subtracting both blank channel and reference channel was applied for data processing. Titration of C5 binding compounds to the C5-immobilized biosensor chip surface led to interactions between C5 and potential binders, and the resulting changes of surface refractive index were sensitively measured by the system.

SPR data was analyzed with the managing software provided by SensiQ and equilibrium dissociation constant (K_{D}) values were determined for each compound at 37° C. Values obtained are presented in Table 31. Where a range of compound concentrations were analyzed, the lowest value obtained is presented.

TABLE 31
Surface plasmon resonance (SPR) data

| | K_{D} |
|---|---|
| Compound No. | (nM) |
| SC0120 | 0.01 |
| SC0116 | 0.02 |
| SC0113 | 0.02 |
| SC0128 | 0.02 |
| SC0154 | 0.04 |
| SC0103 | 0.08 |
| SC0105 | 0.08 |
| SC0106 | 0.08 |
| SC0125 | 0.09 |
| SC0108 | 0.17 |
| SC0115 | 0.18 |
| SC0122 | 0.22 |
| SC0112 | 0.28 |
| SC0107 | 0.36 |
| SC0159 | 0.40 |
| SC0170 | 0.77 |
| SC0174 | 0.78 |
| SC0168 | 0.79 |
| SC0179 | 0.88 |
| SC0104 | 0.89 |
| SC0163 | 0.89 |
| SC0148 | 0.92 |
| SC0178 | 1.07 |
| SC0119 | 1.21 |
| SC0186 | 1.85 |
| SC0172 | 2.25 |
| SC0190 | 2.80 |
| SC0184 | 2.94 |
| SC0183 | 3.88 |

### Example 28. Compound Analysis by Red Blood Cell (RBC) Hemolysis Assay

Sheep red blood cells coated with rabbit anti-sheep erythrocyte antiserum (EA cells; Complement Technology, Tyler, Tex.) were used to assay compound inhibitory activity of the classical complement activation pathway. Briefly, the EA cells were washed once and resuspended in the same volume of GVB++ buffer (Complement Technology, Tyler, Tex.). 25 μL of EA cells were then distributed into each well of 384-well tissue culture plates using Apricot iPipette Pro (Apricot Designs; Covina, Calif.). Compounds were tested in 10 points of final concentrations ranging from 16.67 μM to 1.65 μM in a 6-fold titration series. Compounds were dispensed into 384-well plates from 6.7 mM and 3.35 μM DMSO working stocks using an HP Digital Dispenser (HP; Corvallis, Oreg.). The reactions also contained 1.5% (v/v) C5-depleted human serum (Complement Technology). Hemolysis was induced by addition of human C5 (Complement Technology) at a concentration of 0.5 nM and plates were incubated for 1 hour at 37° C. in a cell culture incubator. The extent of hemolysis was measured by ability of released hemoglobin to catalyze luminol in the presence of hydrogen peroxide. Luminescence was then measured using a plate reader.

Luminescence measurements were used to prepare a dose-response curve. From the curve, the half maximal inhibitory concentration (IC₅₀) for each compound was determined, where the IC₅₀ represents the concentration of each compound needed to reduce red blood cell hemolysis by half. Results are presented in Table 32.

TABLE 32
Red blood cell (RBC) hemolysis assay data

| | IC₅₀ |
|---|---|
| Compound No. | (nM) |
| SC0100 | 0.9 |
| SC0101 | 0.9 |
| SC0102 | 0.9 |
| SC0103 | 1.0 |
| SC0104 | 1.1 |
| SC0105 | 1.1 |
| SC0106 | 1.1 |
| SC0107 | 1.1 |
| SC0108 | 1.2 |
| SC0109 | 1.2 |
| SC0110 | 1.3 |
| SC0111 | 1.3 |
| SC0112 | 1.3 |
| SC0113 | 1.3 |
| SC0114 | 1.3 |
| SC0115 | 1.4 |
| SC0116 | 1.4 |
| SC0117 | 1.4 |
| SC0118 | 1.4 |
| SC0119 | 1.4 |
| SC0120 | 1.5 |
| SC0121 | 1.5 |
| SC0122 | 1.5 |
| SC0123 | 1.5 |
| SC0124 | 1.6 |
| SC0125 | 1.6 |
| SC0126 | 1.6 |
| SC0127 | 1.7 |
| SC0128 | 1.7 |
| SC0129 | 1.7 |
| SC0130 | 1.7 |
| SC0131 | 1.7 |
| SC0132 | 1.7 |
| SC0133 | 1.7 |
| SC0134 | 1.8 |
| SC0135 | 1.8 |
| SC0136 | 1.8 |
| SC0137 | 1.8 |
| SC0138 | 1.8 |
| SC0139 | 1.9 |
| SC0140 | 2.1 |
| SC0141 | 2.1 |
| SC0142 | 2.1 |
| SC0143 | 2.1 |
| SC0144 | 2.1 |
| SC0145 | 2.1 |
| SC0146 | 2.1 |
| SC0147 | 2.2 |
| SC0148 | 2.2 |
| SC0149 | 2.2 |
| SC0150 | 2.2 |
| SC0151 | 2.2 |
| SC0152 | 2.3 |
| SC0153 | 2.3 |
| SC0154 | 2.3 |
| SC0155 | 2.6 |
| SC0156 | 2.6 |
| SC0157 | 2.6 |
| SC0158 | 2.7 |
| SC0159 | 2.7 |
| SC0160 | 2.7 |
| SC0161 | 2.8 |
| SC0162 | 2.8 |
| SC0163 | 2.8 |
| SC0164 | 2.9 |
| SC0165 | 3.0 |
| SC0166 | 3.1 |
| SC0167 | 3.2 |
| SC0168 | 3.3 |
| SC0169 | 3.5 |
| SC0170 | 3.7 |
| SC0171 | 3.7 |
| SC0172 | 3.8 |
| SC0173 | 4.2 |
| SC0174 | 4.3 |
| SC0175 | 4.4 |
| SC0176 | 4.5 |
| SC0177 | 4.7 |
| SC0178 | 4.8 |
| SC0179 | 4.9 |
| SC0180 | 4.9 |
| SC0181 | 5.1 |
| SC0182 | 5.2 |
| SC0183 | 5.3 |
| SC0184 | 5.7 |
| SC0185 | 5.7 |
| SC0186 | 5.8 |
| SC0187 | 6.1 |
| SC0188 | 6.4 |
| SC0189 | 7.0 |
| SC0190 | 7.1 |
| SC0191 | 7.3 |
| SC0192 | 7.8 |
| SC0193 | 8.3 |
| SC0194 | 8.5 |
| SC0195 | 8.5 |
| SC0196 | 8.6 |
| SC0197 | 9.1 |
| SC0198 | 9.7 |
| SC0199 | 9.8 |
| SC0200 | 9.9 |
| SC0201 | 10.0 |
| SC0202 | 10.1 |
| SC0203 | 10.2 |
| SC0204 | 10.9 |
| SC0205 | 11.4 |
| SC0206 | 11.7 |
| SC0207 | 11.8 |
| SC0208 | 12.6 |
| SC0209 | 12.6 |
| SC0210 | 15.1 |
| SC0211 | 15.6 |
| SC0212 | 16.5 |
| SC0213 | 18.2 |
| SC0214 | 18.2 |
| SC0215 | 18.6 |
| SC0216 | 19.6 |
| SC0217 | 25.3 |
| SC0218 | 34.4 |
| SC0219 | 40.0 |
| SC0220 | 42.1 |
| SC0221 | 54.4 |
| SC0222 | 74.1 |
| SC0223 | 96.9 |
| SC0224 | 98.5 |
| SC0225 | 110.3 |
| SC0226 | 132.1 |
| SC0227 | 134.6 |
| SC0228 | 211.0 |
| SC0229 | 371.3 |
| SC0230 | 703.3 |
| SC0231 | 2657.9 |
| SC0232 | 3119.0 |

### Example 29. Compound Analysis by Liquid Chromatography-Mass Spectrometry (LC-MS)

Compounds were analyzed by Liquid chromatography-mass spectrometry (LC-MS) after synthesis to confirm mass-to-charge ratio (m/z). Analytical LCMS was performed by Waters Aquity SDS using a linear gradient of 5% to 100% B over a 5 minute gradient, and UV visualization with a diode array detector. The column used was a C18 Aquity UPLC BEH, 2.1 mm i.d. by 50 mm length, 1.7 μM with flow rate of 0.6 ml/min. Mobile phase A was water and mobile phase B was acetonitrile (0.1% TFA). Results are shown in Table 33.

TABLE 33
LCMS assay data

| | m/z found |
|---|---|
| Compound No. | [M + H] |
| SC0100 | 667.2 |
| SC0101 | 593.2 |
| SC0102 | 531.4 |
| SC0103 | 591.6 |
| SC0104 | 567.1 |
| SC0105 | 618.6 |
| SC0106 | 611.5 |
| SC0107 | 535.6 |
| SC0108 | 556.5 |
| SC0109 | 597.1 |
| SC0110 | 555.8 |
| SC0111 | 597.3 |
| SC0112 | 555.5 |
| SC0113 | 585.3 |
| SC0114 | 565.1 |
| SC0115 | 609.0 |
| SC0116 | 567.0 |
| SC0117 | 653.3 |
| SC0118 | 579.0 |
| SC0119 | 568.1 |
| SC0120 | 586.3 |
| SC0121 | 579.1 |
| SC0122 | 568.4 |
| SC0123 | 597.2 |
| SC0124 | 572.8 |
| SC0125 | 581.1 |
| SC0126 | 593.2 |
| SC0127 | 642.9 |
| SC0128 | 611.4 |
| SC0129 | 625.2 |
| SC0130 | 583.0 |
| SC0131 | 617.9 |
| SC0132 | 569.1 |
| SC0133 | 652.2 |
| SC0134 | 623.2 |
| SC0135 | 524.0 |
| SC0136 | 627.1 |
| SC0137 | 579.1 |
| SC0138 | 531.4 |
| SC0139 | 566.0 |
| SC0140 | 593.1 |
| SC0141 | 593.2 |
| SC0142 | 623.1 |
| SC0143 | 597.2 |
| SC0144 | 607.1 |
| SC0145 | 581.2 |
| SC0146 | 623.2 |
| SC0147 | 611.5 |
| SC0148 | 450.1 |
| SC0149 | 607.2 |
| SC0150 | 573.4 |
| SC0151 | 527.1 |
| SC0152 | 583.1 |
| SC0153 | 611.1 |
| SC0154 | 654.5 |
| SC0155 | 611.4 |
| SC0156 | 656.4 |
| SC0157 | 531.4 |
| SC0158 | 579.1 |
| SC0159 | 551.0 |
| SC0160 | 683.2 |
| SC0161 | 579.1 |
| SC0162 | 600.3 |
| SC0163 | 567.2 |
| SC0164 | 575.0 |
| SC0165 | 625.1 |
| SC0166 | 565.1 |
| SC0167 | 470.3 |
| SC0168 | 546.6 |
| SC0169 | 566.4 |
| SC0170 | 551.0 |
| SC0171 | 620.6 |
| SC0172 | 528.1 |
| SC0173 | 512.1 |
| SC0174 | 533.2 |
| SC0175 | 574.4 |
| SC0176 | 528.1 |
| SC0177 | 620.6 |
| SC0178 | 583.2 |
| SC0179 | 496.0 |
| SC0180 | 483.0 |
| SC0181 | 589.4 |
| SC0182 | 552.3 |
| SC0183 | 570.1 |
| SC0184 | 554.1 |
| SC0185 | 683.1 |
| SC0186 | 568.2 |
| SC0187 | 665.2 |
| SC0188 | 679.1 |
| SC0189 | 514.1 |
| SC0190 | 584.2 |
| SC0191 | 550.1 |
| SC0192 | 496.9 |
| SC0193 | 554.1 |
| SC0194 | 524.0 |
| SC0195 | 443.4 |
| SC0196 | 564.3 |
| SC0197 | 471.0 |
| SC0198 | 498.0 |
| SC0199 | 527.0 |
| SC0200 | 514.0 |
| SC0201 | 554.1 |
| SC0202 | 679.2 |
| SC0203 | 547.2 |
| SC0204 | 541.1 |
| SC0205 | 547.1 |
| SC0206 | 528.1 |
| SC0207 | 538.0 |
| SC0208 | 534.3 |
| SC0209 | 512.1 |
| SC0210 | 512.0 |
| SC0211 | 521.8 |
| SC0212 | 538.0 |
| SC0213 | 474.1 |
| SC0214 | 436.4 |
| SC0215 | 474.1 |
| SC0216 | 665.2 |
| SC0217 | 488.1 |
| SC0218 | 535.2 |
| SC0219 | 437.3 |
| SC0220 | 526.2 |
| SC0221 | 497.0 |
| SC0222 | 501.1 |
| SC0223 | 488.1 |
| SC0224 | 468.0 |
| SC0225 | 602.0 |
| SC0226 | 450.0 |
| SC0227 | 494.2 |
| SC0228 | 452.1 |
| SC0229 | 591.1 |
| SC0230 | 590.2 |
| SC0231 | 306.9 |
| SC0232 | 613.0 |

### Example 30. Permeability Assay

Permeability assays were carried out to provide a preliminary indication of suitability for oral dosing and compound permeability across the blood brain barrier. Unidirectional and bidirectional compound transport across Madin Darby canine kidney (MDCK) cell monolayers was assessed. For unidirectional transport assessment, transport across MDCK wild type (MDCK-WT) cell monolayers was assessed. For bidirectional transport, transport across MDCK-MDR1 cell monolayers was assessed. MDCK-MDR1 cells express the MDR1 gene encoding the P-glycoprotein (P-gp) efflux protein.

MDCK-WT and MDCK-MDR1 cells were plated onto permeable polycarbonate supports in 12-well Costar transwell plates and allowed to grow and differentiate for 3 days. After 3 days in culture, the culture medium (DMEM supplemented with 10% FBS) was removed from both sides of the transwell inserts and cells were rinsed with warm HBSS. After the rinse step, the chambers were filled with warm transport buffer (HBSS containing 10 mM HEPES, 0.25% BSA, pH 7.4) and the plates were pre-incubated at 37° C. for 30 minutes prior to TEER (Trans Epithelial Electric Resistance) measurements.

The buffer in the donor chamber (apical side for A-to-B assay, basolateral side for B-to-A assay) was removed and replaced with the working solution (3 μM test compound and 10 μM for control compounds in transport buffer). The plates were then placed at 37° C. under light agitation. At designated time points (30, 60 and 90 min), an aliquot of transport buffer from the receiver chamber was removed and replenished with fresh transport buffer. Samples were quenched with ice-cold ACN containing internal standard and then centrifuged to pellet protein. Resulting supernatants were further diluted with 50/50 ACN/H₂O (Atenolol diluted in just water) and submitted for LC-MS/MS analysis. Apparent permeability (Pap) values were calculated from duplicate determinations. Atenolol and propranolol were tested as low and moderate permeability references. Bidirectional transport of digoxin was assessed to demonstrate human and canine P-gp activity/expression, while prazosin was assessed to demonstrate human P-gp activity/expression.

Pₐₚₚ values indicate extent of compound permeation across monolayers. Pₐₚₚ values (in centimeters per second or “cm/s”) were calculated using the formula: Pₐₚₚ=[dQ/dt]/[(A)(Ci)(60)], wherein dQ/dt is the net rate of compound appearance in the receiver compartment; “A” is the transwell area measured in centimeters squared; Ci is the initial concentration of compound added to the donor chamber; and 60 is the conversion factor for minutes to seconds. Results with MDCK-WT cells are presented in Table 34.

Efflux ratios were calculated for bidirectional transport across MDCK-MDR1 cell monolayers as an indication of compound efflux by P-gp. The efflux ratio is calculated using the ratio of mean Pₐₚₚ values for B to A transport to mean Pₐₚₚ values for A to B transport [Pₐₚₚ(BA)/Pₐₚₚ(AB)]. Results are presented in Table 34. Efflux ratios greater than 2 indicate active compound efflux.

TABLE 34
Permeability data

| | MDCK-WT | MDCK-MDR |
|---|---|---|
| | Pₐₚₚ A-B | efflux |
| ID# | (10⁻⁶ × cm/s) | ratio |
| CU0616 | 29.0 | 18 |
| CU0261 | 26.1 | 17 |
| CU0140 | 26.0 | 63 |
| CU0558 | 25.2 | 9 |
| CU0501 | 24.2 | 60 |
| CU0689 | 23.8 | 8 |
| CU0579 | 23.5 | 31 |
| CU0560 | 23.0 | 23 |
| CU0555 | 23.0 | 15 |
| CU0510 | 22.7 | 71 |
| SC0108 | 21.7 | 15 |
| CU0160 | 21.6 | 61 |
| CU0506 | 21.1 | 59 |
| SC0102 | 18.2 | |
| SC0113 | 17.9 | |
| CU0534 | 17.4 | |
| CU0529 | 17.1 | 75 |
| CU0145 | 16.8 | 83 |
| CU0526 | 16.8 | |
| CU0511 | 16.6 | |
| SC0112 | 16.4 | 12 |
| SC0110 | 15.3 | 18 |
| SC0100 | 14.3 | 62 |
| SC0164 | 14.3 | |
| SC0117 | 12.6 | |
| SC0155 | 9.9 | |
| CU0513 | 9.6 | |
| SC0120 | 6.6 | 98 |
| SC0109 | 5.7 | 144 |
| SC0123 | 4.8 | |
| SC0101 | 4.7 | |
| CU0520 | 3.9 | |
| CU0521 | 3.9 | |
| SC0149 | 3.5 | |
| SC0144 | 3.0 | |
| SC0146 | 2.6 | |
| SC0111 | 2.5 | |
| CU0136 | 2.4 | |
| SC0134 | 2.2 | |
| SC0171 | 2.0 | |
| SC0169 | 1.8 | |
| CU0533 | 1.7 | |
| SC0177 | 1.3 | |
| SC0156 | 1.3 | |
| CU0518 | 1.1 | |
| CU0523 | 0.7 | |
| SC0147 | 0.7 | |
| SC0114 | 0.1 | |

### Example 31. Synthesis of Substituted Cyclic Urea Compounds and Intermediates

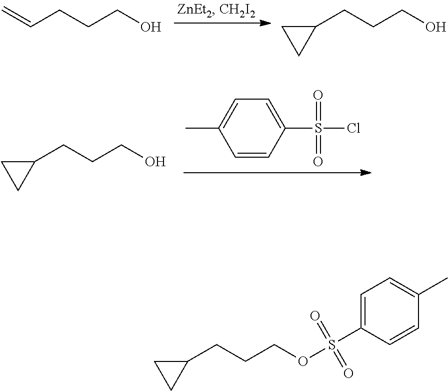

Diethylzinc solution (1.0 M in hexanes, 1.16 L, 1.16 mol) and trifluoroacetic acid (100 g, 0.87 mol) are added to a solution of 4-hexen-1-ol (50.0 g, 0.58 mol) mmol) in dichloromethane (1.5 L) at 0° C. The solution is stirred at 0° C. for 0.5 h then diiodomethane (233 g, 0.87 mol) is added dropwise over 1 h. The resulting mixture is allowed to warm to room temperature and stirred for 14 h. The mixture is quenched with saturated aqueous ammonium chloride solution carefully and then filtered. The filtrate is separated to give aqueous layer and organic layer. The aqueous phase is extracted with dichloromethane. The combined organic layers are washed with brine, dried over anhydrous sodium sulfate and concentrated in vacuo. Crude 3-cyclopropylpropan-1-ol is used in next step without further purification.

Crude 3-cyclopropylpropan-1-ol is dissolved in dichloromethane (500 mL) before addition of triethylamine (78 g, 0.76 mol) and 4-dimethylaminopyridine (4.9 g, 40 mmol). The mixture is cooled to 0° C., then p-toluenesulfonyl chloride (86.6 g, 0.46 mol) is added portion-wise. The solution is allowed to warm to room temperature and stirred for 6 h, then quenched with saturated aqueous sodium bicarbonate solution. The resulting mixture is extracted with dichloromethane. The organic layers are combined, washed with brine, dried over anhydrous sodium sulfate and concentrated to afford crude 3-cyclopropylpropyl 4-methylbenzenesulfonate.

Crude 3-cyclopropylpropyl 4-methylbenzenesulfonate is dissolved in dichloromethane (500 mL), thereto is added diethylzinc solution (1.0 M in hexanes, 350 mL, 0.35 mol) and trifluoroacetic acid (33 g, 0.29 mol). The resulting mixture is cooled to 0° C., and stirred for 0.5 h, then diiodomethane (70.0 g, 0.29 mol) was added dropwise over 0.5 h. The reaction is allowed to warm to room temperature and stirred for 14 h before quenched with saturated aqueous ammonium chloride. The mixture is filtered and the filtrate is separated to give aqueous layer and organic layer. The aqueous phase is extracted with dichloromethane. The organic layers are combined, washed with brine, dried over anhydrous sodium sulfate and concentrated. The residue is purified by flash chromatography over silica gel (5% ethyl acetate in hexanes) to afford compound 3-cyclopropylpropyl 4-methylbenzenesulfonate (69.8 g, 72% yield).

The following compounds are prepared in a similar manner as 3-cyclopropylpropyl 4-methylbenzenesulfonate, as described above.

| Alcohol | Compound |
|---|---|
| 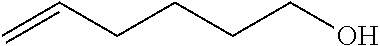 | 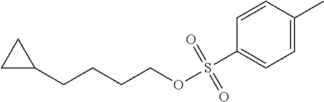 [SMILES: Cc1ccc(S(=O)(=O)OCCCCC2CC2)cc1] |

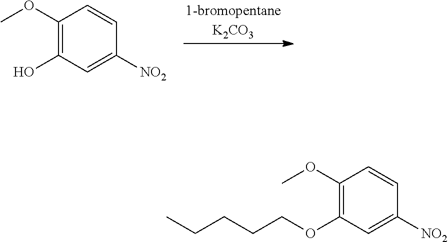 [SMILES: COc1ccc([N+](=O)[O-])cc1O ; CCCCCOc1cc([N+](=O)[O-])ccc1OC]

A solution which included a phenol reactant (2-methoxy-5-nitrophenol, 100.0 g, 0.59 mol) and a bromide reactant (1-bromopentane, 117.2 g, 0.76 mol, 1.3 eq) in a reaction solvent (N,N-dimethylformamide, 1.0 L) is provided. Potassium carbonate (122.5 g, 0.89 mol, 1.5 eq) is added at room temperature. The mixture is heated to 80° C. and stirred for 14 h. The reaction mixture is cooled to room temperature, diluted with water, then extracted with ethyl acetate. The combined organic phases are washed with brine, dried over anhydrous sodium sulfate, then filtered and concentrated in vacuo to a volume of 300 mL. The residue is diluted with hexane (1.0 L) and stirred for 10 minutes to precipitate a white solid. The solid substance is filtered and dried under vacuum to afford 1-methoxy-4-nitro-2-(pentyloxy)benzene (119.7 g) in 85% yield.

The following compounds are prepared in a similar manner as 1-methoxy-4-nitro-2-(pentyloxy)benzene, as described above.

| Phenol | Bromide | Compound |
|---|---|---|
| 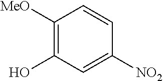 [SMILES: COc1ccc([N+](=O)[O-])cc1O] | 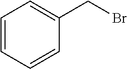 | 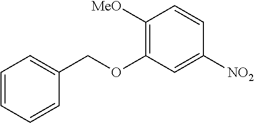 [SMILES: COc1ccc([N+](=O)[O-])cc1OCc1ccccc1] |
| 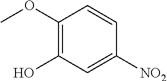 | 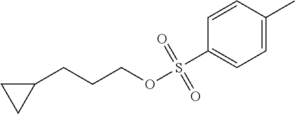 [SMILES: Cc1ccc(S(=O)(=O)OCCCC2CC2)cc1] | 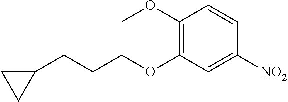 [SMILES: COc1ccc([N+](=O)[O-])cc1OCCCC1CC1] |
| 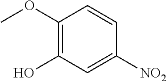 | 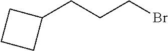 | 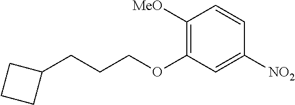 [SMILES: COc1ccc([N+](=O)[O-])cc1OCCCC1CCC1] |
| 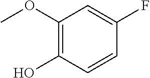 | 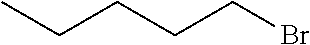 | 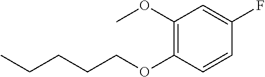 |

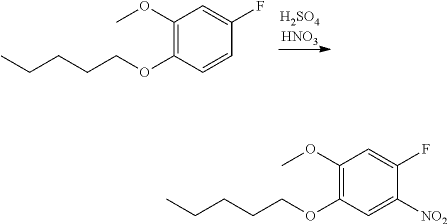 [SMILES: CCCCCOc1ccc(F)cc1OC ; CCCCCOc1cc([N+](=O)[O-])c(F)cc1OC]

Concentrated sulfuric acid (5 mL) is cooled to 0° C. before slowly adding 4-fluoro-2-methoxy-1-(pentyloxy)benzene (500 mg, 2.36 mmol) in portions. Concentrated nitric acid (1 mL) is added slowly dropwise at 0° C. The resulting mixture is stirred at 0° C. for 30 minutes then poured into ice and extracted with ethyl acetate. The combined organic layers are washed with saturated aqueous sodium bicarbonate, then dried over anhydrous sodium sulfate, filtered, and concentrated in vacuo. Purification by column chromatography over silica gel afforded 1-fluoro-5-methoxy-2-nitro-4-(pentyloxy)benzene.

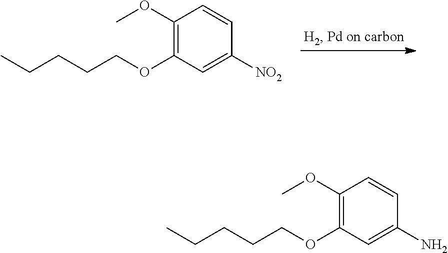 [SMILES: CCCCCOc1cc([N+](=O)[O-])ccc1OC ; CCCCCOc1cc(N)ccc1OC]

1-Methoxy-4-nitro-2-(pentyloxy)benzene (119.5 g, 0.50 mol) is dissolved in methanol (1.5 L), and 10 wt % palladium on carbon (10 g) was added. The mixture is stirred for 14 h under an atmosphere of 1 atm hydrogen. The reaction mixture is filtered through celite, and the filter bed is washed with methanol (500 mL). The filtered solution is concentrated to dryness to afford 4-methoxy-3-(pentyloxy)aniline (95.0 g) in 91% yield.

The following compounds are prepared in a similar manner as 4-methoxy-3-(pentyloxy)aniline, as described above.

| Nitro | Compound |
|---|---|
| 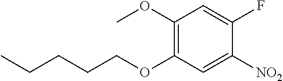 [SMILES: CCCCCOc1cc([N+](=O)[O-])c(F)cc1OC] | 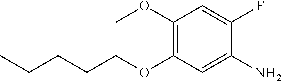 [SMILES: CCCCCOc1cc(N)c(F)cc1OC] |

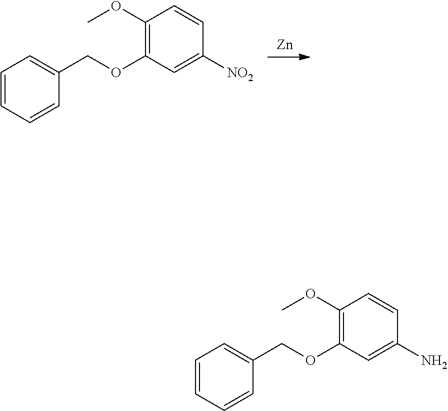

To a solution of 2-(benzyloxy)-1-methoxy-4-nitrobenzene (42.5 g, 164 mmol) in water (87.6 mL) and methanol (876 mL) is added zinc powder (32.1 g, 481 mmol) and acetic acid (93.7 mL, 1.62 mol). The reaction mixture is stirred at 65° C. for 3.5 hours. The mixture is filtered while hot to remove solids and the filtrate is concentrated in vacuo. The residue is dissolved in ethyl acetate and the solution is washed with water, saturated sodium bicarbonate aqueous solution, and brine, then dried over anhydrous sodium sulfate, filtered and concentrated in vacuo. The residue of dark violet oil is used as crude 3-(benzyloxy)-4-methoxyaniline without further purification.

The following compounds are prepared in a similar manner as 3-(benzyloxy)-4-methoxyaniline a described above

| Nitro | Compound |
|---|---|
| 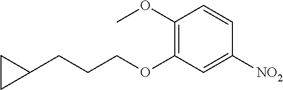 [SMILES: COc1ccc([N+](=O)[O-])cc1OCCCC1CC1] | 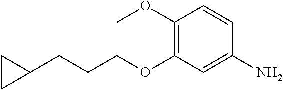 [SMILES: COc1ccc(N)cc1OCCCC1CC1] |
| 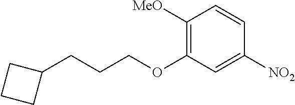 [SMILES: COc1ccc([N+](=O)[O-])cc1OCCCC1CCC1] | 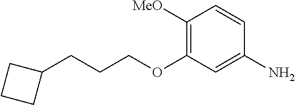 [SMILES: COc1ccc(N)cc1OCCCC1CCC1] |

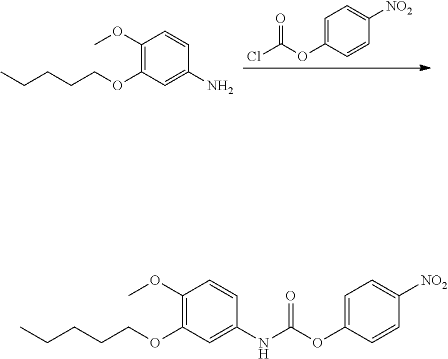 [SMILES: CCCCCOc1cc(N)ccc1OC ; O=C(Cl)Oc1ccc([N+](=O)[O-])cc1 ; CCCCCOc1cc(NC(=O)Oc2ccc([N+](=O)[O-])cc2)ccc1OC]

A solution of 4-nitrophenyl chloroformate (2.1 g, 10 mmol) in anhydrous tetrahydrofuran (10 mL) is cooled to 0° C., then treated with the dropwise addition of a solution of 4-methoxy-3-(pentyloxy)aniline (2.0 g, 9.6 mmol) in anhydrous tetrahydrofuran (20 mL) over 10 min. The mixture is stirred at room temperature for 12 h. The precipitate is collected by filtration and washed with tert-butyl methyl ether/petroleum ether mixture to afford 4-nitrophenyl (4-methoxy-3-(pentyloxy)phenyl)carbamate (2.8 g) in 78% yield.

The following compounds are prepared in a similar manner as 4-nitrophenyl (4-methoxy-3-(pentyloxy)phenyl)carbamate, as described above.

| Amine | Carbamate |
|---|---|
| 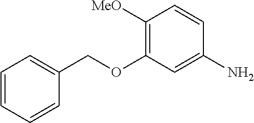 [SMILES: COc1ccc(N)cc1OCc1ccccc1] | 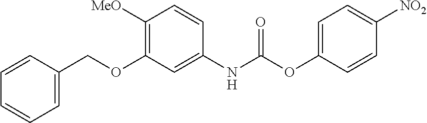 [SMILES: COc1ccc(NC(=O)Oc2ccc([N+](=O)[O-])cc2)cc1OCc1ccccc1] |
| 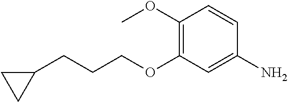 [SMILES: COc1ccc(N)cc1OCCCC1CC1] | 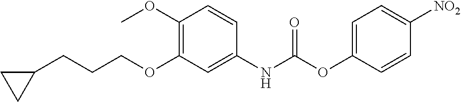 [SMILES: COc1ccc(NC(=O)Oc2ccc([N+](=O)[O-])cc2)cc1OCCCC1CC1] |
| 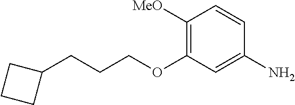 [SMILES: COc1ccc(N)cc1OCCCC1CCC1] | 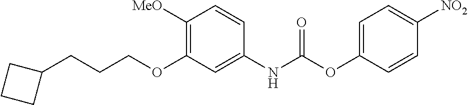 [SMILES: COc1ccc(NC(=O)Oc2ccc([N+](=O)[O-])cc2)cc1OCCCC1CCC1] |
| 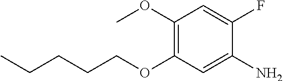 [SMILES: CCCCCOc1cc(N)c(F)cc1OC] | 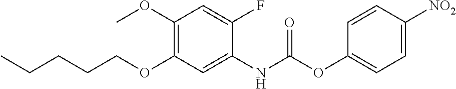 [SMILES: CCCCCOc1cc(NC(=O)Oc2ccc([N+](=O)[O-])cc2)c(F)cc1OC] |

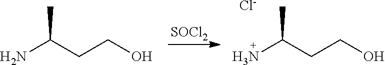

(S)-3-Aminobutan-1-ol (100 g, 1.12 mol) is dissolved in dry dichloromethane (1 L), the solution is cooled to 0° C. Thionyl chloride (200.6 g, 1.69 mol) is added dropwise over 0.5 h. Upon completion, the reaction mixture is stirred at room temperature for 1 h, and at reflux for further 3 h. The reaction mixture is concentrated in vacuo. The residue is triturated with ethyl acetate (100 mL). The solid substance precipitated is filtered and dried over vacuum to afford (S)-4-chlorobutan-2-amine hydrochloride (129.8 g) in 80% yield as an off-white solid.

The following compounds are prepared in a similar manner as (S)-4-chlorobutan-2-amine hydrochloride, as described above.

| Alcohol | Compound |
|---|---|
| 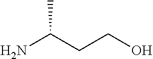 | 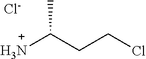 |

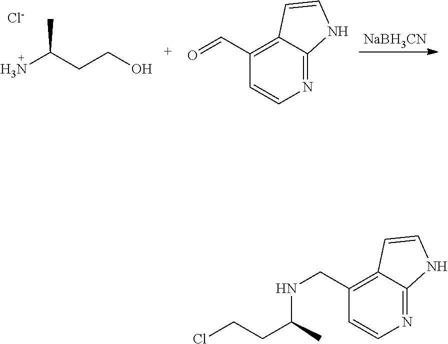

To a solution of (S)-4-chlorobutan-2-amine hydrochloride (1 g, 6.9 mmol) in methanol (10 mL) at 0° C. is added dropwise sodium methoxide (30% methanol solution, 0.38 g, 6.9 mmol). Upon complete addition, 1H-indole-4-carbaldehyde (1.1 g, 7.3 mmol) is added the reaction solution. The mixture is heated at 60° C. for 1.5 h before cooled to 0° C. Glacial acetic acid (0.79 mL, 14 mmol) is added to the reaction mixture followed by sodium cyanoborohydride (0.87 g, 14 mmol). The resulting mixture is stirred for 14 h at room temperature before cooled to 0° C. and quenched with saturated sodium bicarbonate solution. The reaction mixture is extracted with chloroform/isopropanol (v/v 3:1) three times. The combined organic layers are washed with 2 N sodium hydroxide solution, brine, then dried over anhydrous sodium sulfate, filtered and concentrated to give crude (S)—N-((1H-pyrrolo[2,3-b]pyridin-4-yl)methyl)-4-chlorobutan-2-amine.

The following compounds are prepared in a similar manner as (S)—N-((1H-pyrrolo[2,3-b]pyridin-4-yl)methyl)-4-chlorobutan-2-amine, as described above.

| Amine | Carbonyl cpd. | Compound |
|---|---|---|
| 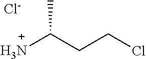 | 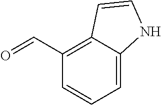 [SMILES: O=Cc1cccc2[nH]ccc12] | 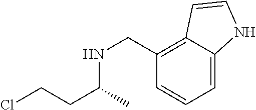 [SMILES: C[C@H](CCCl)NCc1cccc2[nH]ccc12] |
| 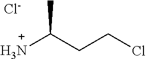 | 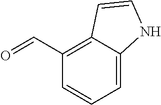 [SMILES: O=Cc1cccc2[nH]ccc12] | 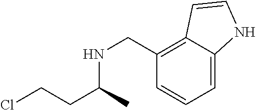 [SMILES: C[C@@H](CCCl)NCc1cccc2[nH]ccc12] |
| 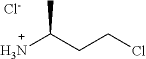 | 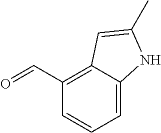 | 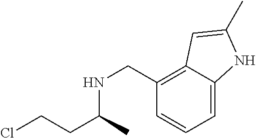 [SMILES: Cc1cc2c(CN[C@@H](C)CCCl)cccc2[nH]1] |
| 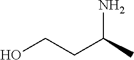 | 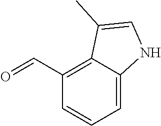 | 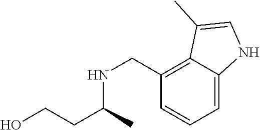 [SMILES: Cc1c[nH]c2cccc(CN[C@@H](C)CCO)c12] |
| 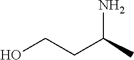 | 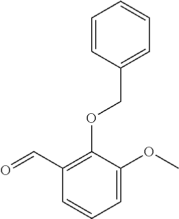 [SMILES: COc1cccc(C=O)c1OCc1ccccc1] | 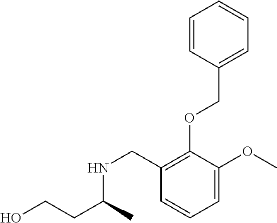 [SMILES: COc1cccc(CN[C@@H](C)CCO)c1OCc1ccccc1] |
| 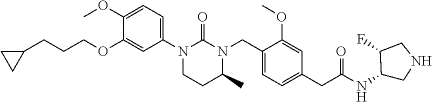 [SMILES: COc1cc(CC(=O)N[C@H]2CNC[C@H]2F)ccc1CN1C(=O)N(c2ccc(OC)c(OCCCC3CC3)c2)CC[C@@H]1C] | 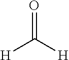 | 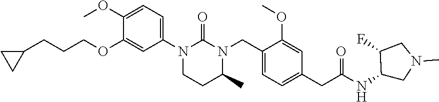 [SMILES: COc1cc(CC(=O)N[C@H]2CN(C)C[C@H]2F)ccc1CN1C(=O)N(c2ccc(OC)c(OCCCC3CC3)c2)CC[C@@H]1C] |
| 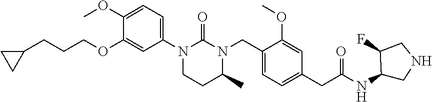 [SMILES: COc1cc(CC(=O)N[C@@H]2CNC[C@@H]2F)ccc1CN1C(=O)N(c2ccc(OC)c(OCCCC3CC3)c2)CC[C@@H]1C] | 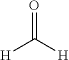 | 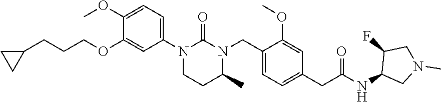 [SMILES: COc1cc(CC(=O)N[C@@H]2CN(C)C[C@@H]2F)ccc1CN1C(=O)N(c2ccc(OC)c(OCCCC3CC3)c2)CC[C@@H]1C] |
| 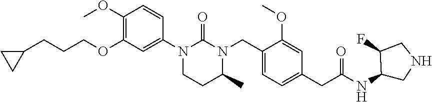 [SMILES: COc1cc(CC(=O)N[C@@H]2CNC[C@@H]2F)ccc1CN1C(=O)N(c2ccc(OC)c(OCCCC3CC3)c2)CC[C@@H]1C] | 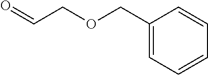 [SMILES: O=CCOCc1ccccc1] | 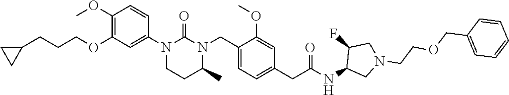 [SMILES: COc1cc(CC(=O)N[C@@H]2CN(CCOCc3ccccc3)C[C@@H]2F)ccc1CN1C(=O)N(c2ccc(OC)c(OCCCC3CC3)c2)CC[C@@H]1C] |
| 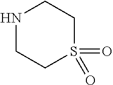 | 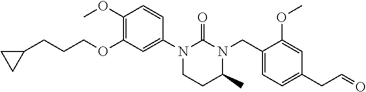 [SMILES: COc1cc(CC=O)ccc1CN1C(=O)N(c2ccc(OC)c(OCCCC3CC3)c2)CC[C@@H]1C] | 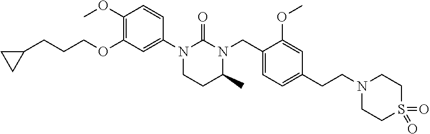 [SMILES: COc1cc(CCN2CCS(=O)(=O)CC2)ccc1CN1C(=O)N(c2ccc(OC)c(OCCCC3CC3)c2)CC[C@@H]1C] |
| 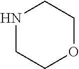 | 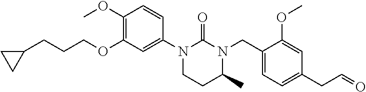 [SMILES: COc1cc(CC=O)ccc1CN1C(=O)N(c2ccc(OC)c(OCCCC3CC3)c2)CC[C@@H]1C] | 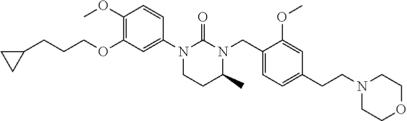 [SMILES: COc1cc(CCN2CCOCC2)ccc1CN1C(=O)N(c2ccc(OC)c(OCCCC3CC3)c2)CC[C@@H]1C] |
| 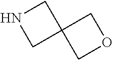 | 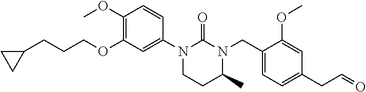 [SMILES: COc1cc(CC=O)ccc1CN1C(=O)N(c2ccc(OC)c(OCCCC3CC3)c2)CC[C@@H]1C] | 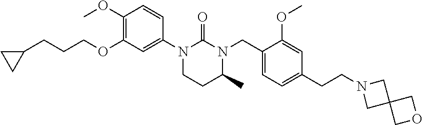 [SMILES: COc1cc(CCN2CC3(COC3)C2)ccc1CN1C(=O)N(c2ccc(OC)c(OCCCC3CC3)c2)CC[C@@H]1C] |

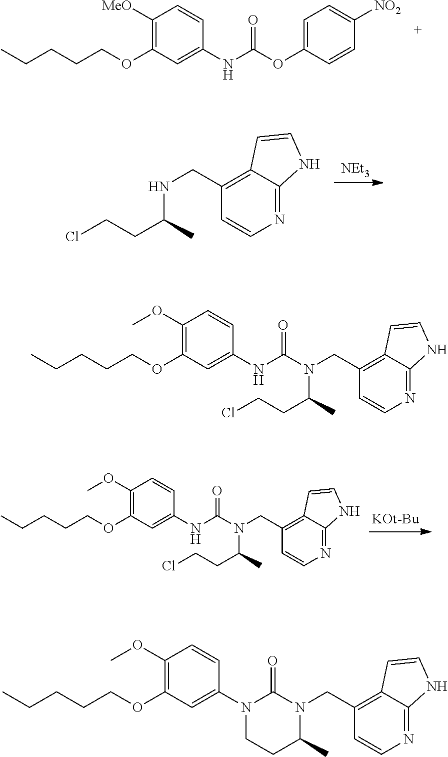

To a solution of (S)—N-((1H-pyrrolo[2,3-b]pyridin-4-yl)methyl)-4-chlorobutan-2-amine in tetrahydrofuran (27 mL) at 0° C. are added 4-nitrophenyl (4-methoxy-3-(pentyloxy)phenyl)carbamate (2.9 g, 7.6 mmol) and triethylamine (1.9 mL, 14 mmol). The reaction mixture is allowed to warm to room temperature and stirred for 3 h before quenched by saturated sodium bicarbonate solution. The resulting mixture is extracted with ethyl acetate three times. The combined organic layers are washed with 2 N aqueous sodium hydroxide solution, brine, then dried over anhydrous sodium sulfate, filtered and concentrated to afford crude (S)-1-((1H-pyrrolo[2,3-b]pyridin-4-yl)methyl)-1-(4-chlorobutan-2-yl)-3-(4-methoxy-3-(pentyloxy)phenyl)urea.

(S)-1-((1H-pyrrolo[2,3-b]pyridin-4-yl)methyl)-1-(4-chlorobutan-2-yl)-3-(4-methoxy-3-(pentyloxy)phenyl)urea is dissolved in tetrahydrofuran (150 mL). The solution is cooled at 0° C. before addition of potassium tert-butoxide (2.3 g, 21 mmol) portion-wise. The reaction mixture is stirred at room temperature for 3 h before cooled to 0° C. Saturated aqueous ammonium chloride solution is added to quench the reaction and the resulting mixture is extracted with ethyl acetate 3 times. The combined organic layers are washed with brine, dried over anhydrous sodium sulfate, filtered and concentrated in vacuo. The residue is purified by column chromatography over silica gel to afford (S)-3-((1H-pyrrolo[2,3-b]pyridin-4-yl)methyl)-1-(4-methoxy-3-(pentyloxy)phenyl)-4-methyltetrahydropyrimidin-2(1H)-one (1.9 g, 64%) as a yellow solid.

The following compounds are prepared in a similar manner as compound (S)-3-((1H-pyrrolo[2,3-b]pyridin-4-yl)methyl)-1-(4-methoxy-3-(pentyloxy)phenyl)-4-methyltetrahydropyrimidin-2(1H)-one, as described above.

| Amine | Nitro | Compound |
|---|---|---|
| 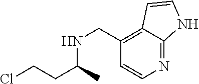 [SMILES: C[C@@H](CCCl)NCc1ccnc2[nH]ccc12] | 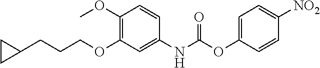 [SMILES: COc1ccc(NC(=O)Oc2ccc([N+](=O)[O-])cc2)cc1OCCCC1CC1] | 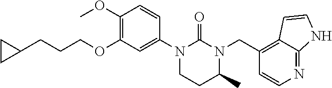 [SMILES: COc1ccc(N2CC[C@H](C)N(Cc3ccnc4[nH]ccc34)C2=O)cc1OCCCC1CC1] |
| 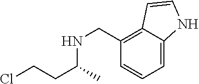 [SMILES: C[C@H](CCCl)NCc1cccc2[nH]ccc12] | 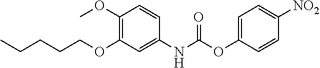 [SMILES: CCCCCOc1cc(NC(=O)Oc2ccc([N+](=O)[O-])cc2)ccc1OC] | 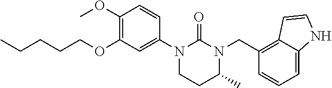 [SMILES: CCCCCOc1cc(N2CC[C@@H](C)N(Cc3cccc4[nH]ccc34)C2=O)ccc1OC] |
| 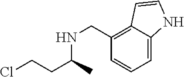 [SMILES: C[C@@H](CCCl)NCc1cccc2[nH]ccc12] | 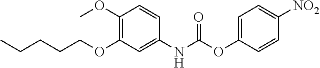 [SMILES: CCCCCOc1cc(NC(=O)Oc2ccc([N+](=O)[O-])cc2)ccc1OC] | 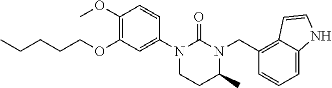 [SMILES: CCCCCOc1cc(N2CC[C@H](C)N(Cc3cccc4[nH]ccc34)C2=O)ccc1OC] |
| 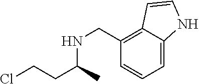 [SMILES: C[C@@H](CCCl)NCc1cccc2[nH]ccc12] | 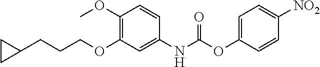 [SMILES: COc1ccc(NC(=O)Oc2ccc([N+](=O)[O-])cc2)cc1OCCCC1CC1] | 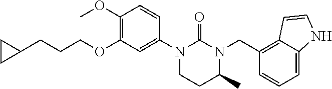 [SMILES: COc1ccc(N2CC[C@H](C)N(Cc3cccc4[nH]ccc34)C2=O)cc1OCCCC1CC1] |
| 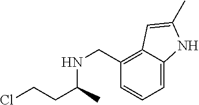 [SMILES: Cc1cc2c(CN[C@@H](C)CCCl)cccc2[nH]1] | 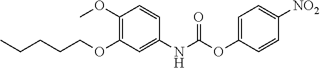 [SMILES: CCCCCOc1cc(NC(=O)Oc2ccc([N+](=O)[O-])cc2)ccc1OC] | 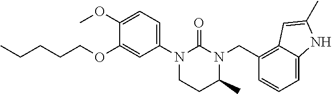 [SMILES: CCCCCOc1cc(N2CC[C@H](C)N(Cc3cccc4[nH]c(C)cc34)C2=O)ccc1OC] |
| 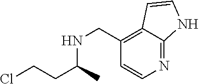 [SMILES: C[C@@H](CCCl)NCc1ccnc2[nH]ccc12] | 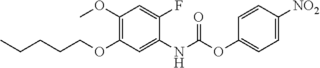 [SMILES: CCCCCOc1cc(NC(=O)Oc2ccc([N+](=O)[O-])cc2)c(F)cc1OC] | 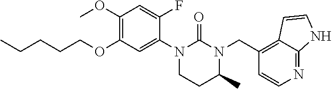 [SMILES: CCCCCOc1cc(N2CC[C@H](C)N(Cc3ccnc4[nH]ccc34)C2=O)c(F)cc1OC] |

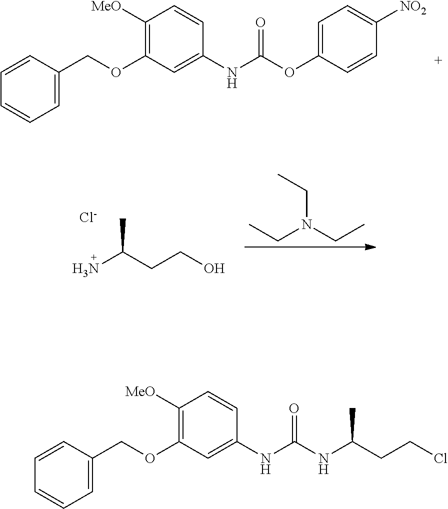

4-Nitrophenyl (3-(benzyloxy)-4-methoxyphenyl)carbamate (331 g, 0.84 mol) and (S)-4-chlorobutan-2-amine hydrochloride (163 g, 1:2.6 mol) are dissolved in dichloromethane (3 L). The mixture is cooled to 0° C. and triethylamine (255 g, 2.52 mol) is added dropwise while maintaining the internal temperature at 0-5° C. Upon complete addition, the mixture is allowed to warm to room temperature and stirred for 0.5 h. The reaction is quenched by addition of water (2 L). Organic layer is separated from aqueous layer, then washed with brine, dried over anhydrous sodium sulfate, filtered and concentrated in vacuo. The residue is triturated with a mixture of hexane/ethyl acetate (v/v 4/1, 2 L). The solid substance is collected by filtration and dried under vacuum to afford (S)-1-(3-(benzyloxy)-4-methoxyphenyl)-3-(4-chlorobutan-2-yl)urea (264 g, 90% yield) as a yellow solid.

The following compounds are prepared in a similar manner as (S)-1-(3-(benzyloxy)-4-methoxyphenyl)-3-(4-chlorobutan-2-yl)urea, as described above.

| Carbamate | Amine | Compound |
|---|---|---|
| 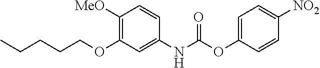 [SMILES: CCCCCOc1cc(NC(=O)Oc2ccc([N+](=O)[O-])cc2)ccc1OC] | 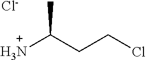 | 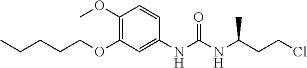 [SMILES: CCCCCOc1cc(NC(=O)N[C@@H](C)CCCl)ccc1OC] |
| 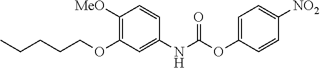 [SMILES: CCCCCOc1cc(NC(=O)Oc2ccc([N+](=O)[O-])cc2)ccc1OC] | 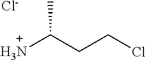 | 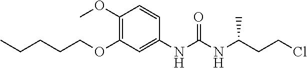 [SMILES: CCCCCOc1cc(NC(=O)N[C@H](C)CCCl)ccc1OC] |
| 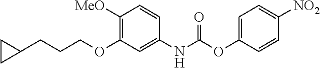 [SMILES: COc1ccc(NC(=O)Oc2ccc([N+](=O)[O-])cc2)cc1OCCCC1CC1] | 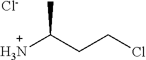 | 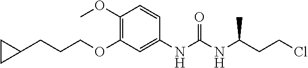 [SMILES: COc1ccc(NC(=O)N[C@@H](C)CCCl)cc1OCCCC1CC1] |
| 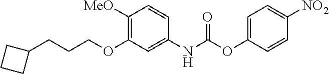 [SMILES: COc1ccc(NC(=O)Oc2ccc([N+](=O)[O-])cc2)cc1OCCCC1CCC1] | 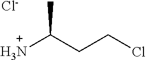 | 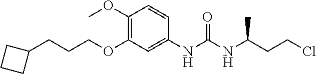 [SMILES: COc1ccc(NC(=O)N[C@@H](C)CCCl)cc1OCCCC1CCC1] |
| 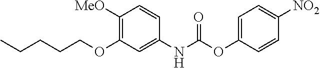 [SMILES: CCCCCOc1cc(NC(=O)Oc2ccc([N+](=O)[O-])cc2)ccc1OC] | 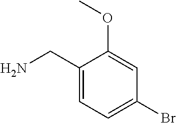 | 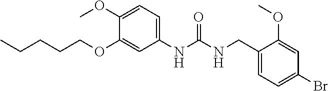 [SMILES: CCCCCOc1cc(NC(=O)NCc2ccc(Br)cc2OC)ccc1OC] |
| 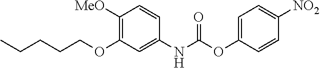 [SMILES: CCCCCOc1cc(NC(=O)Oc2ccc([N+](=O)[O-])cc2)ccc1OC] | 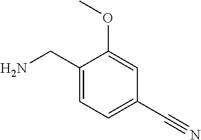 | 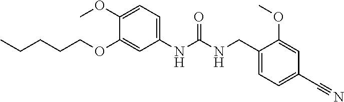 [SMILES: CCCCCOc1cc(NC(=O)NCc2ccc(C#N)cc2OC)ccc1OC] |
| 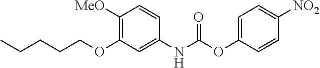 [SMILES: CCCCCOc1cc(NC(=O)Oc2ccc([N+](=O)[O-])cc2)ccc1OC] | 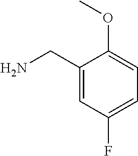 | 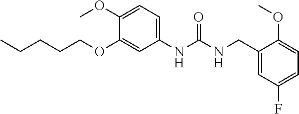 [SMILES: CCCCCOc1cc(NC(=O)NCc2cc(F)ccc2OC)ccc1OC] |
| 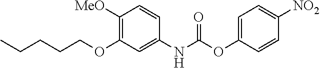 [SMILES: CCCCCOc1cc(NC(=O)Oc2ccc([N+](=O)[O-])cc2)ccc1OC] | 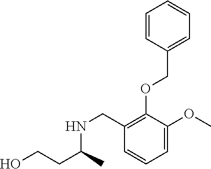 [SMILES: COc1cccc(CN[C@@H](C)CCO)c1OCc1ccccc1] | 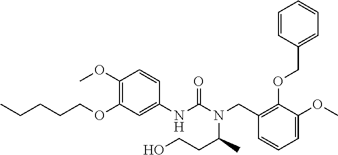 [SMILES: CCCCCOc1cc(NC(=O)N(Cc2cccc(OC)c2OCc2ccccc2)[C@@H](C)CCO)ccc1OC] |
| 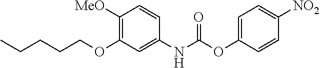 [SMILES: CCCCCOc1cc(NC(=O)Oc2ccc([N+](=O)[O-])cc2)ccc1OC] | 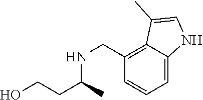 [SMILES: Cc1c[nH]c2cccc(CN[C@@H](C)CCO)c12] | 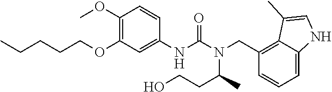 [SMILES: CCCCCOc1cc(NC(=O)N(Cc2cccc3[nH]cc(C)c23)[C@@H](C)CCO)ccc1OC] |
| 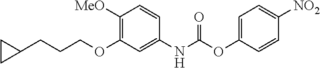 [SMILES: COc1ccc(NC(=O)Oc2ccc([N+](=O)[O-])cc2)cc1OCCCC1CC1] | 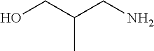 | 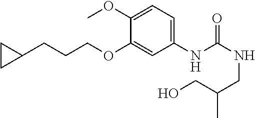 [SMILES: COc1ccc(NC(=O)NCC(C)CO)cc1OCCCC1CC1] |

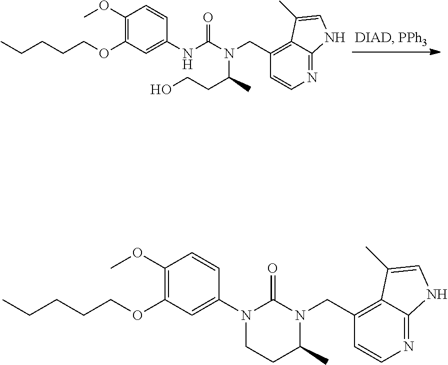 [SMILES: CCCCCOc1cc(NC(=O)N(Cc2ccnc3[nH]cc(C)c23)[C@@H](C)CCO)ccc1OC ; CCCCCOc1cc(N2CC[C@H](C)N(Cc3ccnc4[nH]cc(C)c34)C2=O)ccc1OC]

To a stirring solution of (S)-1-(4-hydroxybutan-2-yl)-3-(4-methoxy-3-(pentyloxy) phenyl)-1-((3-methyl-1H-indol-4-yl)methyl)urea (1.29 g, 2.75 mmol) and triphenylphosphine (0.86 g, 3.3 mmol) in anhydrous tetrahydrofuran (10 mL) at 0° C., is added diisopropyl azodicarboxylate (0.83 g, 4.12 mmol). The resulting solution is slowly warmed to a room temperature and then stirred for 8 h. The reaction is quenched with water and the mixture is extracted with ethyl acetate three times. The combined organic layers are washed with brine, dried over anhydrous sodium sulfate, filtered and concentrated in vacuo. The residue is purified by column chromatography over silica gel using 5% (v/v) methanol in dichloromethane (with 0.1% v/v ammonium hydroxide additive) as eluent to afford (S)-1-(4-methoxy-3-(pentyloxy)phenyl)-4-methyl-3-((3-methyl-1H-indol-4-yl)methyl)tetrahydropyrimidin-2(1H)-one (905 mg, 65% yield).

The following compounds are prepared in a similar manner as afford (S)-1-(4-methoxy-3-(pentyloxy)phenyl)-4-methyl-3-((3-methyl-1H-indol-4-yl)methyl)tetrahydropyrimidin-2(1H)-one, as described above.

| Linear urea | Cyclized urea |
|---|---|
| 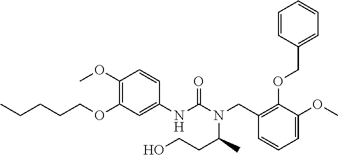 [SMILES: CCCCCOc1cc(NC(=O)N(Cc2cccc(OC)c2OCc2ccccc2)[C@@H](C)CCO)ccc1OC] | 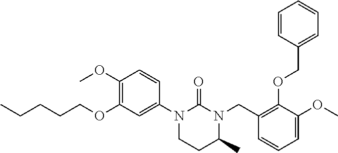 [SMILES: CCCCCOc1cc(N2CC[C@H](C)N(Cc3cccc(OC)c3OCc3ccccc3)C2=O)ccc1OC] |
| 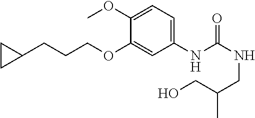 [SMILES: COc1ccc(NC(=O)NCC(C)CO)cc1OCCCC1CC1] | 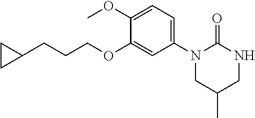 [SMILES: COc1ccc(N2CC(C)CNC2=O)cc1OCCCC1CC1] |

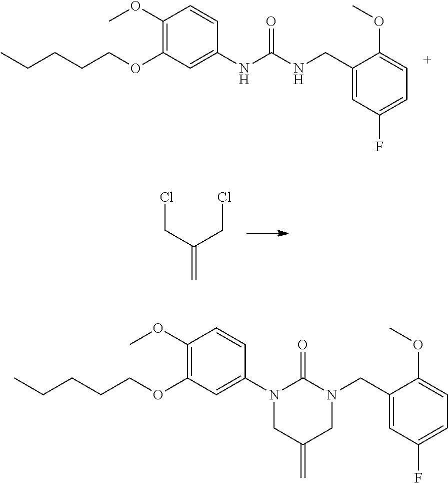

To a solution of 1-(5-fluoro-2-methoxybenzyl)-3-(4-methoxy-3-(pentyloxy)phenyl)urea (9.00 g, 23.1 mmol) in tetrahydrofuran (158 mL) at 0° C., sodium hydride (60% dispersion in mineral oil, 2.77 g, 69.2 mmol) is added. After it was stirred at 0° C. for 5 min, 3-chloro-2-chloromethyl-1-propene (6.74 mL, 57.6 mmol) is added to the reaction mixture, which is allowed to warm to room temperature and then heated to reflux for 4 h. The reaction mixture is cooled to room temperature and quenched with water. The mixture is extracted with ethyl acetate three times and the combined organic layers are washed with saturated ammonium chloride aqueous solution, brine, then dried over anhydrous sodium sulfate, filtered and concentrated in vacuo. The residue is suspended in toluene (ice cold) and then filtered. The filtrate is concentrated and the residue was purified over silica gel by flash chromatography (60% ethyl acetate in heptanes) to give 1-(5-fluoro-2-methoxybenzyl)-3-(4-methoxy-3-(pentyloxy)phenyl)-5-methylenetetrahydropyrimidin-2(1H)-one (3.67 g, 36% yield) as an oil.

The following compounds are prepared in a similar manner as 1-(5-fluoro-2-methoxybenzyl)-3-(4-methoxy-3-(pentyloxy)phenyl)-5-methylenetetrahydropyrimidin-2(1H)-one, as described above.

| | Dichloro | |
|---|---|---|
| Linear urea | cpd. | Cyclized urea |
| 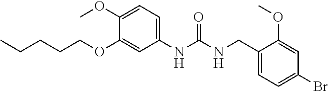 [SMILES: CCCCCOc1cc(NC(=O)NCc2ccc(Br)cc2OC)ccc1OC] | 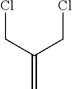 | 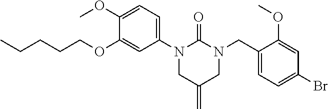 [SMILES: C=C1CN(Cc2ccc(Br)cc2OC)C(=O)N(c2ccc(OC)c(OCCCCC)c2)C1] |
| 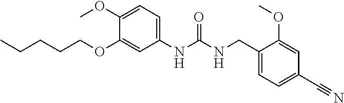 [SMILES: CCCCCOc1cc(NC(=O)NCc2ccc(C#N)cc2OC)ccc1OC] | 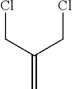 | 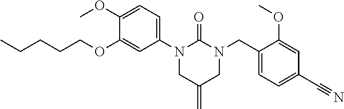 [SMILES: C=C1CN(Cc2ccc(C#N)cc2OC)C(=O)N(c2ccc(OC)c(OCCCCC)c2)C1] |
| 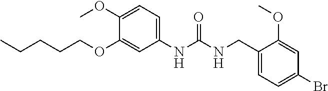 [SMILES: CCCCCOc1cc(NC(=O)NCc2ccc(Br)cc2OC)ccc1OC] | 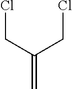 | 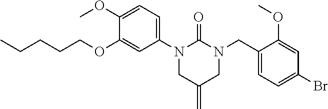 [SMILES: C=C1CN(Cc2ccc(Br)cc2OC)C(=O)N(c2ccc(OC)c(OCCCCC)c2)C1] |
| 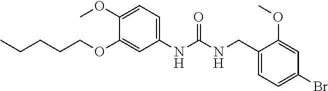 [SMILES: CCCCCOc1cc(NC(=O)NCc2ccc(Br)cc2OC)ccc1OC] | 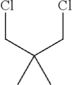 | 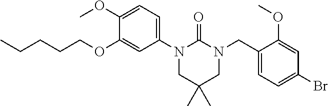 [SMILES: CCCCCOc1cc(N2CC(C)(C)CN(Cc3ccc(Br)cc3OC)C2=O)ccc1OC] |
| 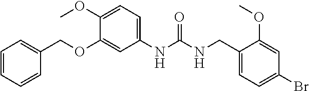 [SMILES: COc1cc(Br)ccc1CNC(=O)Nc1ccc(OC)c(OCc2ccccc2)c1] | 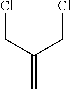 | 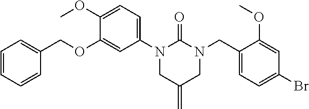 [SMILES: C=C1CN(Cc2ccc(Br)cc2OC)C(=O)N(c2ccc(OC)c(OCc3ccccc3)c2)C1] |

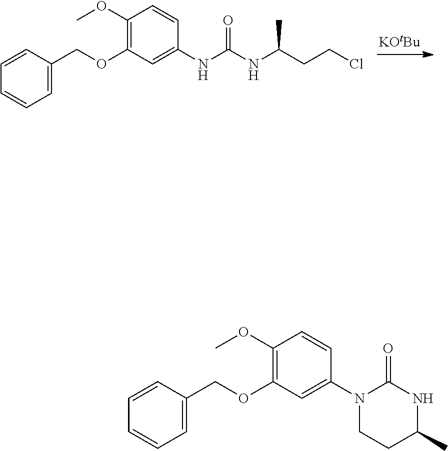 [SMILES: COc1ccc(NC(=O)N[C@@H](C)CCCl)cc1OCc1ccccc1 ; COc1ccc(N2CC[C@H](C)NC2=O)cc1OCc1ccccc1 ; "KOtBu"]

Powder potassium tert-butoxide (212.2 g. 1.89 mol) is added portion-wise to a solution of (S)-1-(3-(benzyloxy)-4-methoxyphenyl)-3-(4-chlorobutan-2-yl)urea (230.0 g, 0.63 mol) in N,N-dimethylformamide (2.5 L) at room temperature. The reaction mixture is stirred at room temperature for 1 h. The mixture is quenched with water and then extracted with dichloromethane 3 times. The combined organic phases are dried over anhydrous sodium sulfate, filtered and concentrated in vacuo. The residue is triturated with a mixture of ethyl acetate/hexane (v/v 1/1). The solid substance is filtered and dried under vacuum to afford (S)-1-(3-(benzyloxy)-4-methoxyphenyl)-4-methyltetrahydropyrimidin-2(1H)-one (135 g, 66% yield) as an off-white solid.

The following compounds are prepared in a similar manner as (S)-1-(3-(benzyloxy)-4-methoxyphenyl)-4-methyltetrahydropyrimidin-2(1H)-one, as described above.

| Linear urea | Cyclic urea |
|---|---|
| 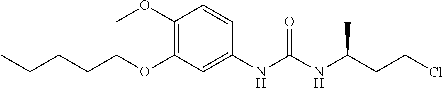 [SMILES: CCCCCOc1cc(NC(=O)N[C@@H](C)CCCl)ccc1OC] | 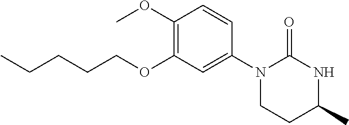 [SMILES: CCCCCOc1cc(N2CC[C@H](C)NC2=O)ccc1OC] |
| 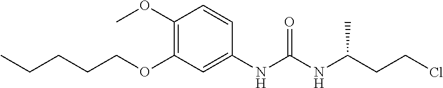 [SMILES: CCCCCOc1cc(NC(=O)N[C@H](C)CCCl)ccc1OC] | 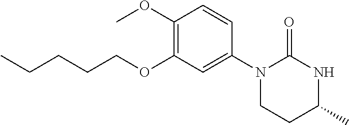 [SMILES: CCCCCOc1cc(N2CC[C@@H](C)NC2=O)ccc1OC] |
| 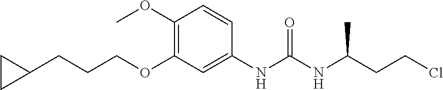 [SMILES: COc1ccc(NC(=O)N[C@@H](C)CCCl)cc1OCCCC1CC1] | 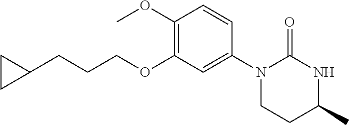 [SMILES: COc1ccc(N2CC[C@H](C)NC2=O)cc1OCCCC1CC1] |
| 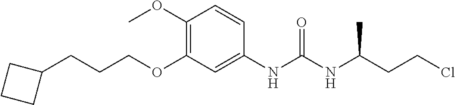 [SMILES: COc1ccc(NC(=O)N[C@@H](C)CCCl)cc1OCCCC1CCC1] | 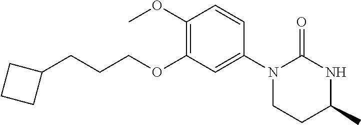 [SMILES: COc1ccc(N2CC[C@H](C)NC2=O)cc1OCCCC1CCC1] |

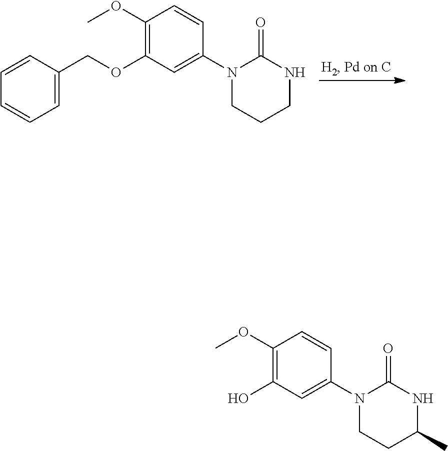

The solution of (S)-1-(3-(benzyloxy)-4-methoxyphenyl)-4-methyltetrahydropyrimidin-2(1H)-one (0.32 g, 1.0 mmol) in methanol (5.0 mL) is degassed with nitrogen for 5 min. To this mixture is added 10% palladium on carbon (53 mg, 50 μmol) and the resulting mixture is degassed with hydrogen for 30 min. The mixture is stirred under 1 atmosphere of hydrogen for 16 h at room temperature, then filtered through celite and washed with methanol. The filtrate is concentrated in vacuo to give (S)-1-(3-hydroxy-4-methoxyphenyl)-4-methyltetrahydropyrimidin-2(1H)-one (0.24 g, 100%) as a yellow solid.

The following compounds are prepared in a similar manner as (S)-1-(3-hydroxy-4-methoxyphenyl)-4-methyltetrahydropyrimidin-2(1H)-one, as described above.

| Substrate | Compound |
|---|---|
| 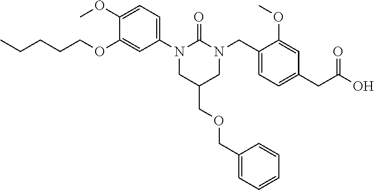 [SMILES: CCCCCOc1cc(N2CC(COCc3ccccc3)CN(Cc3ccc(CC(=O)O)cc3OC)C2=O)ccc1OC] | 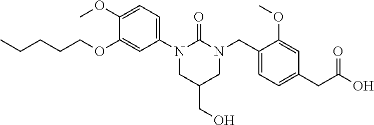 [SMILES: CCCCCOc1cc(N2CC(CO)CN(Cc3ccc(CC(=O)O)cc3OC)C2=O)ccc1OC] |
| | racemate |
| 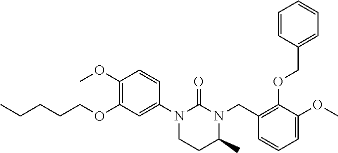 [SMILES: CCCCCOc1cc(N2CC[C@H](C)N(Cc3cccc(OC)c3OCc3ccccc3)C2=O)ccc1OC] | 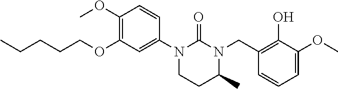 [SMILES: CCCCCOc1cc(N2CC[C@H](C)N(Cc3cccc(OC)c3O)C2=O)ccc1OC] |
| 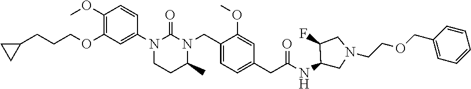 [SMILES: COc1cc(CC(=O)N[C@@H]2CN(CCOCc3ccccc3)C[C@@H]2F)ccc1CN1C(=O)N(c2ccc(OC)c(OCCCC3CC3)c2)CC[C@@H]1C] | 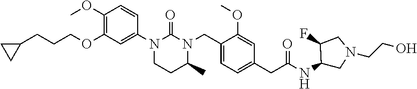 [SMILES: COc1cc(CC(=O)N[C@@H]2CN(CCO)C[C@@H]2F)ccc1CN1C(=O)N(c2ccc(OC)c(OCCCC3CC3)c2)CC[C@@H]1C] |
| 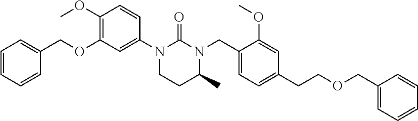 [SMILES: COc1cc(CCOCc2ccccc2)ccc1CN1C(=O)N(c2ccc(OC)c(OCc3ccccc3)c2)CC[C@@H]1C] | 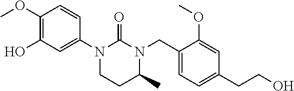 [SMILES: COc1ccc(N2CC[C@H](C)N(Cc3ccc(CCO)cc3OC)C2=O)cc1O] |
| 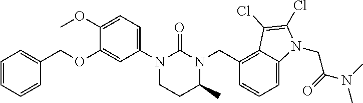 [SMILES: COc1ccc(N2CC[C@H](C)N(Cc3cccc4c3c(Cl)c(Cl)n4CC(=O)N(C)C)C2=O)cc1OCc1ccccc1] | 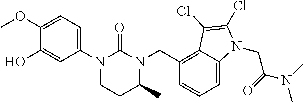 |

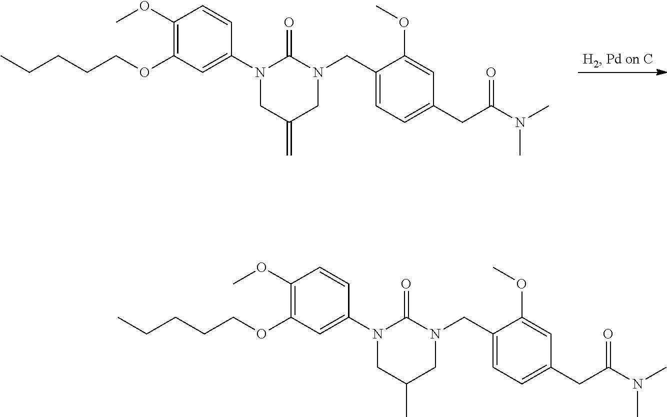

The solution of 2-(3-methoxy-4-((3-(4-methoxy-3-(pentyloxy)phenyl)-5-methylene-2-oxotetrahydropyrimidin-1(2H)-yl)methyl)phenyl)-N,N-dimethylacetamide (82 mg, 160 μmol) in methanol (5.0 mL) is degassed with nitrogen for 5 mi. To this mixture is added 10% palladium on carbon (30.0 mg, 73.0 μmol) and the resulting mixture is degassed with hydrogen for 30 min. The mixture is stirred under 1 atmosphere of hydrogen for 16 h at room temperature, then filtered through celite and washed with methanol. The filtrate is concentrated in vacuo to give 2-(3-methoxy-4-((3-(4-n-methoxy-3-(pentyloxy)phenyl)-5-methyl-2-oxotetrahydropyrimidin-1(2H)-yl)methyl)phenyl)-N,N-dimethylacetamide (75 mg, 86%0) as a dark yellow amorphous solid.

The following compounds are prepared in a similar manner as 2-(3-methoxy-4-((3-(4-methoxy-3-(pentyloxy)phenyl)-5-methyl-2-oxotetrahydropyrimidin-1(2H)-yl)methyl)phenyl)-N,N-dimethylacetamide, as described above.

| Substrate | Compound |
|---|---|
| 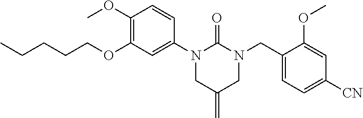 [SMILES: C=C1CN(Cc2ccc(C#N)cc2OC)C(=O)N(c2ccc(OC)c(OCCCCC)c2)C1] | 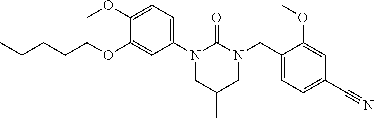 [SMILES: CCCCCOc1cc(N2CC(C)CN(Cc3ccc(C#N)cc3OC)C2=O)ccc1OC] |
| | racemate |
| 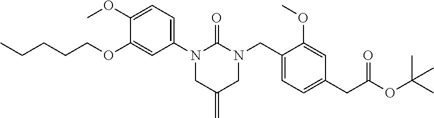 [SMILES: C=C1CN(Cc2ccc(CC(=O)OC(C)(C)C)cc2OC)C(=O)N(c2ccc(OC)c(OCCCCC)c2)C1] | 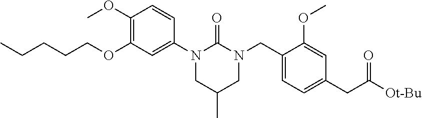 [SMILES: CCCCCOc1cc(N2CC(C)CN(Cc3ccc(CC(=O)OC(C)(C)C)cc3OC)C2=O)ccc1OC] |

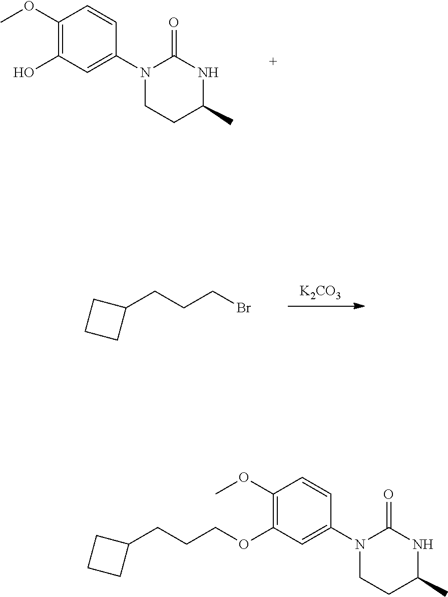

To (S)-1-(3-hydroxy-4-methoxyphenyl)-4-methyltetrahydropyrimidin-2(1H)-one (0.24 g, 1.0 mmol) in N,N-dimethylformamide (5 mL) were added (3-bromopropyl)cyclobutene (0.21 g, 1.2 mmol) and potassium carbonate (0.17 g, 1.2 mmol). The reaction mixture is stirred at 90° C. for 2 h before cooled to room temperature. The reaction mixture is suspended in ethyl acetate (50 mL), washed with saturated ammonium chloride aqueous solution, water, brine, then dried over anhydrous sodium sulfate, filtered and concentrated in vacuo. The residue is purified over silica gel by column chromatography to give (S)-1-(3-(3-cyclobutylpropoxy)-4-methoxyphenyl)-4-methyltetrahydropyrimidin-2(1H)-one (0.28 g, 85% yield) as a yellow solid.

The following compounds are prepared in a similar manner as (S)-1-(3-(3-cyclobutylpropoxy)-4-methoxyphenyl)-4-methyltetrahydropyrimidin-2(1H)-one, as described above.

| Phenol | Reactant | Compound |
|---|---|---|
| 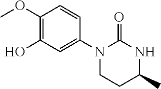 | 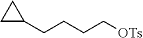 [SMILES: Cc1ccc(S(=O)(=O)OCCCCC2CC2)cc1] | 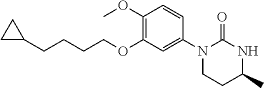 [SMILES: COc1ccc(N2CC[C@H](C)NC2=O)cc1OCCCCC1CC1] |
| 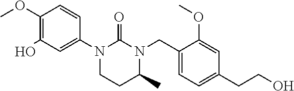 [SMILES: COc1ccc(N2CC[C@H](C)N(Cc3ccc(CCO)cc3OC)C2=O)cc1O] | 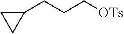 [SMILES: Cc1ccc(S(=O)(=O)OCCCC2CC2)cc1] | 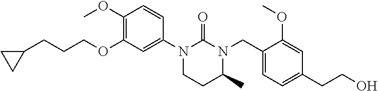 [SMILES: COc1cc(CCO)ccc1CN1C(=O)N(c2ccc(OC)c(OCCCC3CC3)c2)CC[C@@H]1C] |
| 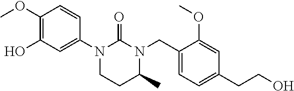 [SMILES: COc1ccc(N2CC[C@H](C)N(Cc3ccc(CCO)cc3OC)C2=O)cc1O] | 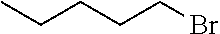 | 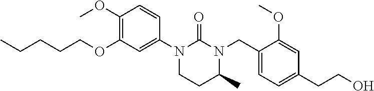 [SMILES: CCCCCOc1cc(N2CC[C@H](C)N(Cc3ccc(CCO)cc3OC)C2=O)ccc1OC] |
| 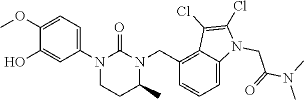 | 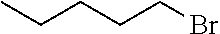 | 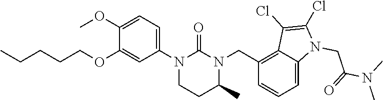 [SMILES: CCCCCOc1cc(N2CC[C@H](C)N(Cc3cccc4c3c(Cl)c(Cl)n4CC(=O)N(C)C)C2=O)ccc1OC] |

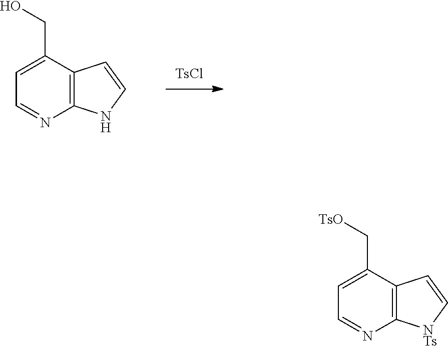 [SMILES: OCc1ccnc2[nH]ccc12 ; "TsCl" ; Cc1ccc(S(=O)(=O)OCc2ccnc3c2ccn3S(=O)(=O)c2ccc(C)cc2)cc1]

Sodium hydride (60% suspension in oil, 3.6 g, 89 mmol) is added portion-wise to a solution of 1H-pyrrolo[2,3-b]pyridin-4-yl)methanol (6.3 g, 42.6 mmol) in tetrahydrofuran (100 mL) at 0° C. over 10 min. The reaction mixture is stirred for 0.5 h at room temperature, then re-cooled to 0° C., and 4-toluenesulfonyl chloride (17.0 g, 89 mmol) is added. The reaction mixture is allowed to warm to room temperature and stirred for 2 h. The mixture is quenched with water (200 mL), and extracted with ethyl acetate. The combined organic phases are washed with brine, dried over anhydrous sodium sulfate, filtered and concentrated to dryness. The residue is triturated with ethyl acetate/hexane (v/v 1/10, 50 mL). The solid substance is filtered and dried under vacuum to afford 1-tosyl-1H-pyrrolo[2,3-b]pyridin-4-yl)methyl 4-methylbenzenesulfonate (10.5 g, 55% yield) as an off-white solid.

The following compounds are prepared in a similar manner as 1-tosyl-H-pyrrolo[2,3-b]pyridin-4-yl)methyl 4-methylbenzenesulfonate as described above.

| Substrate | Compound |
|---|---|
| 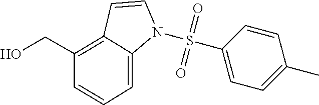 [SMILES: Cc1ccc(S(=O)(=O)n2ccc3c(CO)cccc32)cc1] | 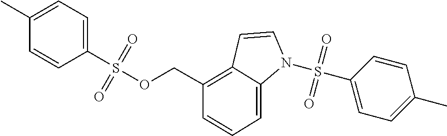 |

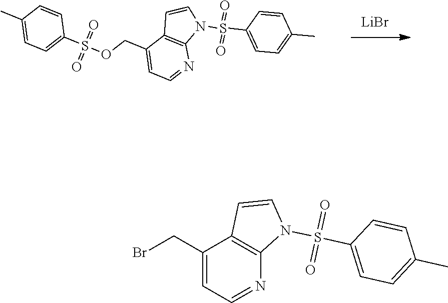

A mixture of 1-tosyl-1H-pyrrolo[2,3-b]pyridin-4-yl)methyl 4-methylbenzenesulfonate (5.1 g, 11.2 mmol) and lithium bromide (2.7 g, 14.5 mmol) in tetrahydrofuran (50 mL) is stirred at room temperature for 4 h. The mixture is quenched with water, then extracted with ethyl acetate. The combined organic phases are washed with brine, dried over anhydrous sodium sulfate, filtered and concentrated to afford 4-(bromomethyl)-1-tosyl-1H-pyrrolo[2,3-b]pyridine (4.0 g, 98% yield) as a white solid.

The following compounds are prepared in a similar manner as 4-(bromomethyl)-1-tosyl-1H-pyrrolo[2,3-b]pyridine, as described above.

| Substrate | Compound |
|---|---|
| 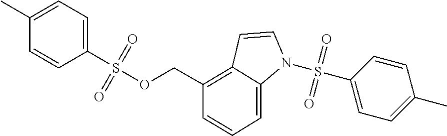 | 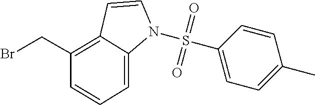 [SMILES: Cc1ccc(S(=O)(=O)n2ccc3c(CBr)cccc32)cc1] |

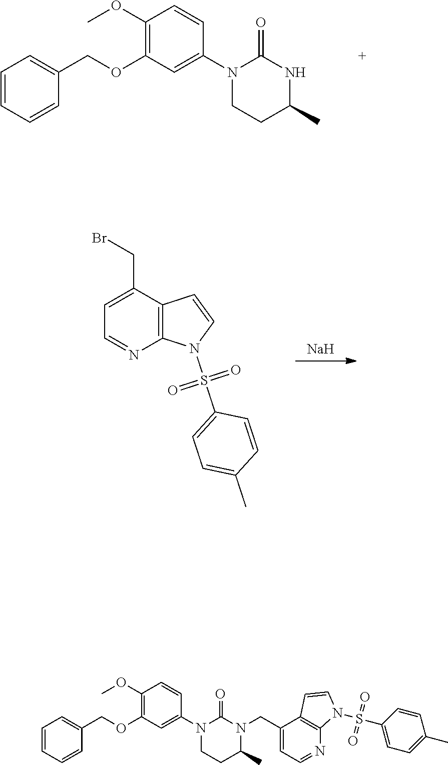

To a suspension of (S)-(3-(benzyloxy)-4-methoxyphenyl)-4-methyltetrahydropyrimidin-2(1H)-one (4.40 g, 13.5 mmol) in dry tetrahydrofuran (89.9 mL) under a nitrogen atmosphere is added sodium hydride (60% dispersion in mineral oil, 1.08 g, 27.0 mmol) in one portion. The reaction is then heated to 40° C. for 3 hours. 4-(Bromomethyl)-1-tosyl-1H-pyrrolo[2,3-b]pyridine (5.17 g, 14.2 mmol) in tetrahydrofuran (30.0 mL) is added dropwise over 1 hour at 40° C. The reaction mixture is stirred for 5 minutes, then is cooled to room temperature and quenched by the slow addition of 1 N HCl. Brine is added and the aqueous layer was extracted with ethyl acetate twice. The combined organic layers are dried over anhydrous sodium sulfate, filtered and concentrated to dryness. The residue is taken in ethyl acetate to precipitate unreacted starting material. The resulting suspension is filtered and the filtrate is concentrated to dryness. The residue is purified by column chromatography over silica gel (50% ethyl acetate in dichloromethane) to afford (S)-1-(3-(benzyloxy)-4-methoxyphenyl)-4-methyl-3-((1-tosyl-1H-pyrrolo[2,3-b]pyridin-4-yl)methyl)tetrahydropyrimidin-2(1H)-one) (2.65 g, 42% yield) as a white foam.

The following compounds are prepared in a similar manner as (S)-1-(3-(benzyloxy)-4-methoxyphenyl)-4-methyl-3-((1-tosyl-1H-pyrrolo[2,3-b]pyridin-4-yl)methyl)tetrahydropyrimidin-2(1H)-one), as described above.

| Substrate | Bromide | Compound |
|---|---|---|
| 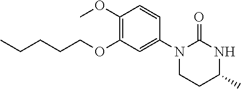 [SMILES: CCCCCOc1cc(N2CC[C@@H](C)NC2=O)ccc1OC] | 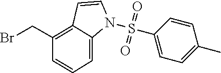 [SMILES: Cc1ccc(S(=O)(=O)n2ccc3c(CBr)cccc32)cc1] | 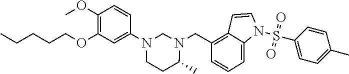 [SMILES: CCCCCOc1cc(N2CC[C@@H](C)N(Cc3cccc4c3ccn4S(=O)(=O)c3ccc(C)cc3)C2)ccc1OC] |
| 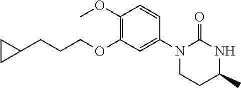 [SMILES: COc1ccc(N2CC[C@H](C)NC2=O)cc1OCCCC1CC1] | 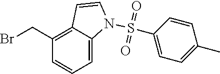 [SMILES: Cc1ccc(S(=O)(=O)n2ccc3c(CBr)cccc32)cc1] | 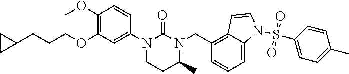 [SMILES: COc1ccc(N2CC[C@H](C)N(Cc3cccc4c3ccn4S(=O)(=O)c3ccc(C)cc3)C2=O)cc1OCCCC1CC1] |
| 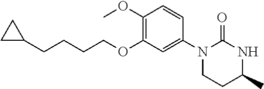 [SMILES: COc1ccc(N2CC[C@H](C)NC2=O)cc1OCCCCC1CC1] | 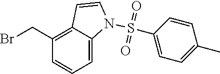 [SMILES: Cc1ccc(S(=O)(=O)n2ccc3c(CBr)cccc32)cc1] | 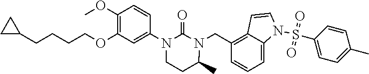 [SMILES: COc1ccc(N2CC[C@H](C)N(Cc3cccc4c3ccn4S(=O)(=O)c3ccc(C)cc3)C2=O)cc1OCCCCC1CC1] |
| 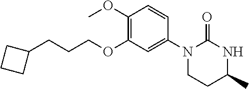 [SMILES: COc1ccc(N2CC[C@H](C)NC2=O)cc1OCCCC1CCC1] | 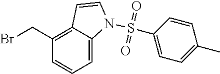 [SMILES: Cc1ccc(S(=O)(=O)n2ccc3c(CBr)cccc32)cc1] | 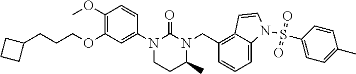 [SMILES: COc1ccc(N2CC[C@H](C)N(Cc3cccc4c3ccn4S(=O)(=O)c3ccc(C)cc3)C2=O)cc1OCCCC1CCC1] |
| 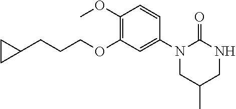 [SMILES: COc1ccc(N2CC(C)CNC2=O)cc1OCCCC1CC1] | 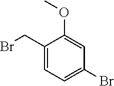 | 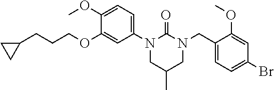 [SMILES: COc1cc(Br)ccc1CN1CC(C)CN(c2ccc(OC)c(OCCCC3CC3)c2)C1=O] |

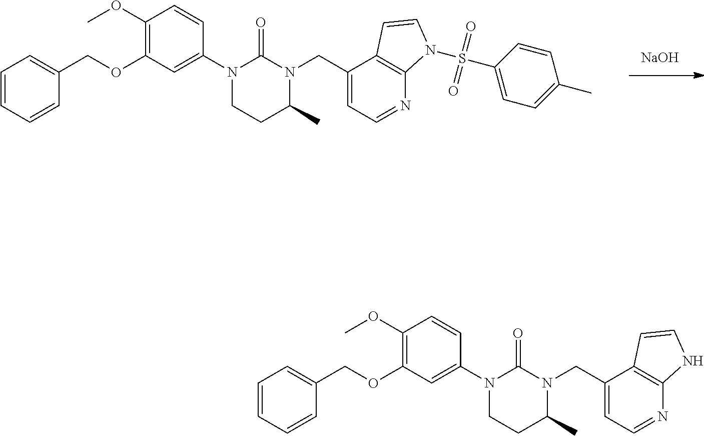 [SMILES: COc1ccc(N2CC[C@H](C)N(Cc3ccnc4c3ccn4S(=O)(=O)c3ccc(C)cc3)C2=O)cc1OCc1ccccc1 ; "NaOH" ; COc1ccc(N2CC[C@H](C)N(Cc3ccnc4[nH]ccc34)C2=O)cc1OCc1ccccc1]

To a solution of ((S)-1-(3-(benzyloxy)-4-methoxyphenyl)-4-methyl-3-((1-tosyl-1H-pyrrolo[2,3-b]pyridin-4-yl)methyl)tetrahydropyrimidin-2(1H)-one) (2.65 g, 4.34 mmol) in tetrahydrofuran (43.4 mL) and methanol (43.4 mL) is added 50% aqueous sodium hydroxide solution (5.78 mL). The reaction mixture is stirred for 10 minutes, then concentrated under vacuum. Saturated aqueous ammonium chloride solution is added and the aqueous layer is extracted with dichloromethane twice. The combined organic layers are dried over anhydrous sodium sulfate, filtered, and evaporated to dryness to provide (S)-3-((1H-pyrrolo[2,3-b]pyridin-4-yl)methyl)-1-(3-(benzyloxy)-4-methoxyphenyl)-4-methyltetrahydropyrimidin-2(1H)-one (2.02 g, 97% yield) as an orange foam that is used without any further purification.

The following compounds are prepared in a similar manner as (S)-3-((1H-pyrrolo[2,3-b]pyridin-4-yl)methyl)-1-(3-(benzyloxy)-4-methoxyphenyl)-4-methyltetrahydropyrimidin-2(1H)-one, as described above.

| Substrate | Compound |
|---|---|
| 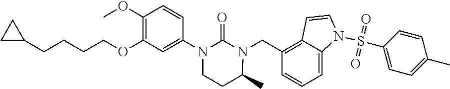 [SMILES: COc1ccc(N2CC[C@H](C)N(Cc3cccc4c3ccn4S(=O)(=O)c3ccc(C)cc3)C2=O)cc1OCCCCC1CC1] | 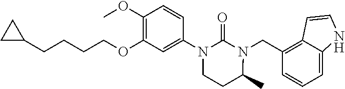 [SMILES: COc1ccc(N2CC[C@H](C)N(Cc3cccc4[nH]ccc34)C2=O)cc1OCCCCC1CC1] |
| 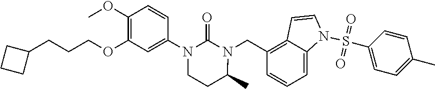 [SMILES: COc1ccc(N2CC[C@H](C)N(Cc3cccc4c3ccn4S(=O)(=O)c3ccc(C)cc3)C2=O)cc1OCCCC1CCC1] | 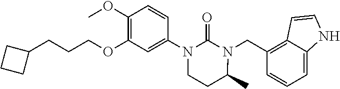 [SMILES: COc1ccc(N2CC[C@H](C)N(Cc3cccc4[nH]ccc34)C2=O)cc1OCCCC1CCC1] |

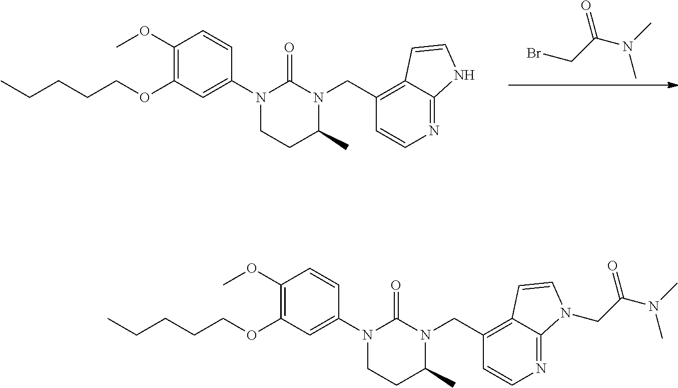

To a suspension of sodium hydride (60 wt % in mineral oil, 98 mg, 4.1 mmol) in N,N-dimethylformamide (4 mL) at 0° C. is slowly added a solution of (S)-3-((1H-pyrrolo[2,3-b]pyridin-4-yl)methyl)-1-(4-methoxy-3-(pentyloxy)phenyl)-4-methyltetrahydropyrimidin-2(1H)-one (0.89 g, 2.0 mmol) in N,N-dimethylformamide (6 mL). The mixture is stirred at room temperature for 30 min before addition of a solution of 2-bromo-N,N-dimethylacetamide (0.44 mL, 4.1 mmol) in N,N-dimethylformamide (1 mL). The reaction is warmed to room temperature and stirred for 3 h. Water is slowly added to quench the reaction, and the resulting mixture is extracted twice with ethyl acetate. The combined organic layers are washed with brine, dried over anhydrous sodium sulfate, and concentrated in vacuo. The residue is purified by column chromatography over silica gel to afford (S)-2-(4-((3-(4-methoxy-3-(pentyloxy)phenyl)-6-methyl-2-oxotetrahydropyrimidin-1(2H)-yl)methyl)-1H-pyrrolo[2,3-b]pyridin-1-yl)-N,N-dimethylacetamide (0.76 g, 72% yield).

The following compounds are prepared in a similar manner as (S)-2-(4-((3-(4-methoxy-3-(pentyloxy)phenyl)-6-methyl-2-oxotetrahydropyrimidin-1(2H)-yl)methyl)-1H-pyrrolo[2,3-b]pyridin-1-yl)-N,N-dimethylacetamide, as described above.

| Heterocycle | Halide | Compound |
|---|---|---|
| 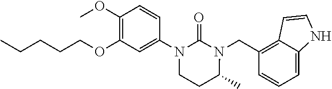 [SMILES: CCCCCOc1cc(N2CC[C@@H](C)N(Cc3cccc4[nH]ccc34)C2=O)ccc1OC] | 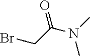 | 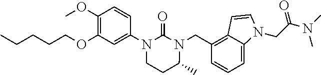 [SMILES: CCCCCOc1cc(N2CC[C@@H](C)N(Cc3cccc4c3ccn4CC(=O)N(C)C)C2=O)ccc1OC] |
| 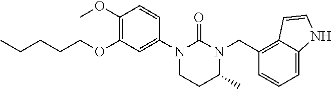 [SMILES: CCCCCOc1cc(N2CC[C@@H](C)N(Cc3cccc4[nH]ccc34)C2=O)ccc1OC] | 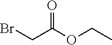 | 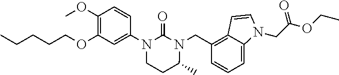 [SMILES: CCCCCOc1cc(N2CC[C@@H](C)N(Cc3cccc4c3ccn4CC(=O)OCC)C2=O)ccc1OC] |
| 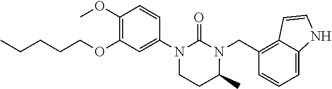 [SMILES: CCCCCOc1cc(N2CC[C@H](C)N(Cc3cccc4[nH]ccc34)C2=O)ccc1OC] | 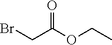 | 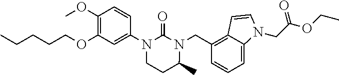 [SMILES: CCCCCOc1cc(N2CC[C@H](C)N(Cc3cccc4c3ccn4CC(=O)OCC)C2=O)ccc1OC] |
| 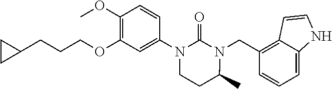 [SMILES: COc1ccc(N2CC[C@H](C)N(Cc3cccc4[nH]ccc34)C2=O)cc1OCCCC1CC1] | 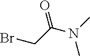 | 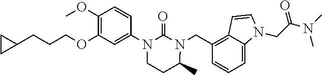 [SMILES: COc1ccc(N2CC[C@H](C)N(Cc3cccc4c3ccn4CC(=O)N(C)C)C2=O)cc1OCCCC1CC1] |
| 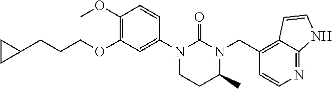 [SMILES: COc1ccc(N2CC[C@H](C)N(Cc3ccnc4[nH]ccc34)C2=O)cc1OCCCC1CC1] | 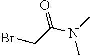 | 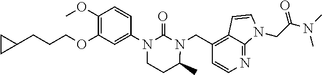 [SMILES: COc1ccc(N2CC[C@H](C)N(Cc3ccnc4c3ccn4CC(=O)N(C)C)C2=O)cc1OCCCC1CC1] |
| 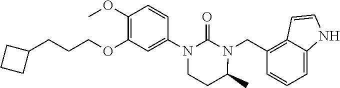 [SMILES: COc1ccc(N2CC[C@H](C)N(Cc3cccc4[nH]ccc34)C2=O)cc1OCCCC1CCC1] | 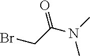 | 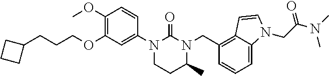 [SMILES: COc1ccc(N2CC[C@H](C)N(Cc3cccc4c3ccn4CC(=O)N(C)C)C2=O)cc1OCCCC1CCC1] |
| 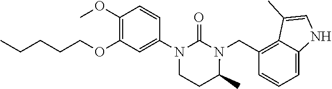 [SMILES: CCCCCOc1cc(N2CC[C@H](C)N(Cc3cccc4[nH]cc(C)c34)C2=O)ccc1OC] | 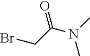 | 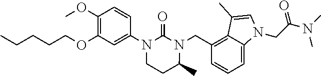 [SMILES: CCCCCOc1cc(N2CC[C@H](C)N(Cc3cccc4c3c(C)cn4CC(=O)N(C)C)C2=O)ccc1OC] |
| 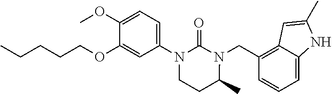 [SMILES: CCCCCOc1cc(N2CC[C@H](C)N(Cc3cccc4[nH]c(C)cc34)C2=O)ccc1OC] | 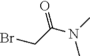 | 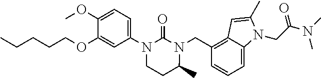 [SMILES: CCCCCOc1cc(N2CC[C@H](C)N(Cc3cccc4c3cc(C)n4CC(=O)N(C)C)C2=O)ccc1OC] |
| 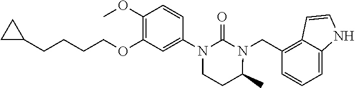 [SMILES: COc1ccc(N2CC[C@H](C)N(Cc3cccc4[nH]ccc34)C2=O)cc1OCCCCC1CC1] | 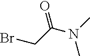 | 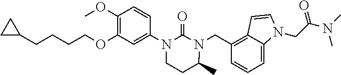 [SMILES: COc1ccc(N2CC[C@H](C)N(Cc3cccc4c3ccn4CC(=O)N(C)C)C2=O)cc1OCCCCC1CC1] |
| 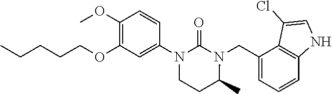 [SMILES: CCCCCOc1cc(N2CC[C@H](C)N(Cc3cccc4[nH]cc(Cl)c34)C2=O)ccc1OC] | 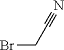 | 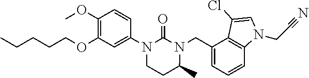 [SMILES: CCCCCOc1cc(N2CC[C@H](C)N(Cc3cccc4c3c(Cl)cn4CC#N)C2=O)ccc1OC] |

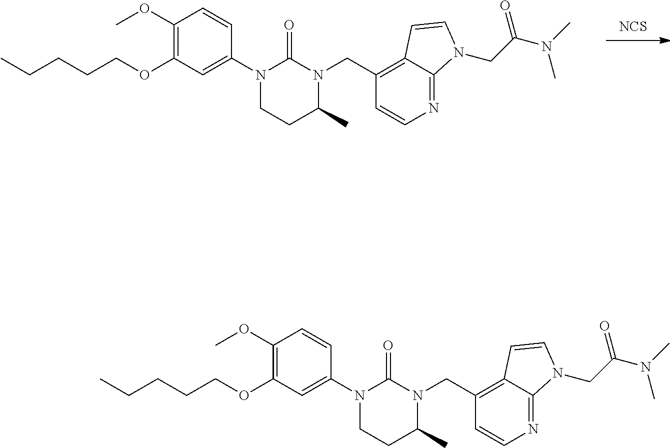

A solution of, (S)-2-(4-((3-(4-methoxy-3-(pentyloxy)phenyl)-6-methyl-2-oxotetrahydropyrimidin-1(2H)-yl)methyl)-1H-pyrrolo[2,3-b]pyridin-1-yl)-N,N-dimethylacetamide (87 mg, 0.17 mmol) and N-chlorosuccinimide (25 mg, 0.18 mmol, 1.1 eq) in tetrahydrofuran (1.7 mL) is stirred at 50° C. for 1 h. The reaction solution is concentrated in vacuo. The residue is purified by prep HPLC to afford compound (S)-2-(3-chloro-4-((3-(4-methoxy-3-(pentyloxy)phenyl)-6-methyl-2-oxotetrahydropyrimidin-1(2H)-yl)methyl)-1H-pyrrolo[2,3-b]pyridin-1-yl)-N,N-dimethylacetamide (87 mg, 0.13 mmol) as a white solid.

The following compounds are prepared in a similar manner as (S)-2-(3-chloro-4-((3-(4-methoxy-3-(pentyloxy)phenyl)-6-methyl-2-oxotetrahydropyrimidin-1(2H)-yl)methyl)-1H-pyrrolo[2,3-b]pyridin-1-yl)-N,N-dimethylacetamide, as described above.

| Substrate | Compound |
|---|---|
| 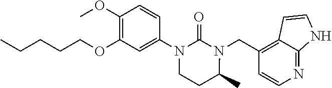 [SMILES: CCCCCOc1cc(N2CC[C@H](C)N(Cc3ccnc4[nH]ccc34)C2=O)ccc1OC] | 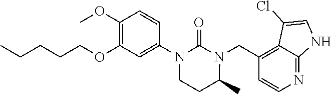 [SMILES: CCCCCOc1cc(N2CC[C@H](C)N(Cc3ccnc4[nH]cc(Cl)c34)C2=O)ccc1OC] |
| 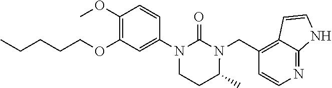 [SMILES: CCCCCOc1cc(N2CC[C@@H](C)N(Cc3ccnc4[nH]ccc34)C2=O)ccc1OC] | 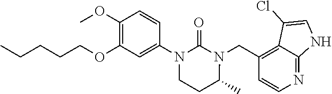 [SMILES: CCCCCOc1cc(N2CC[C@@H](C)N(Cc3ccnc4[nH]cc(Cl)c34)C2=O)ccc1OC] |
| 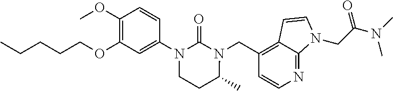 [SMILES: CCCCCOc1cc(N2CC[C@@H](C)N(Cc3ccnc4c3ccn4CC(=O)N(C)C)C2=O)ccc1OC] | 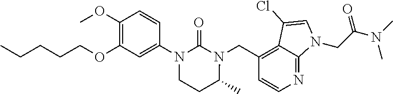 [SMILES: CCCCCOc1cc(N2CC[C@@H](C)N(Cc3ccnc4c3c(Cl)cn4CC(=O)N(C)C)C2=O)ccc1OC] |
| 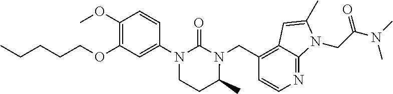 [SMILES: CCCCCOc1cc(N2CC[C@H](C)N(Cc3ccnc4c3cc(C)n4CC(=O)N(C)C)C2=O)ccc1OC] | 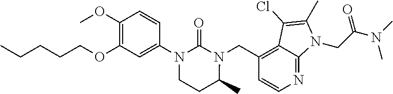 [SMILES: CCCCCOc1cc(N2CC[C@H](C)N(Cc3ccnc4c3c(Cl)c(C)n4CC(=O)N(C)C)C2=O)ccc1OC] |
| 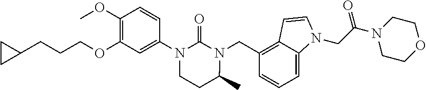 [SMILES: COc1ccc(N2CC[C@H](C)N(Cc3cccc4c3ccn4CC(=O)N3CCOCC3)C2=O)cc1OCCCC1CC1] | 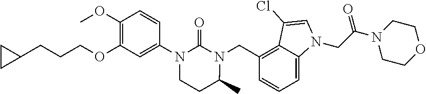 [SMILES: COc1ccc(N2CC[C@H](C)N(Cc3cccc4c3c(Cl)cn4CC(=O)N3CCOCC3)C2=O)cc1OCCCC1CC1] |
| 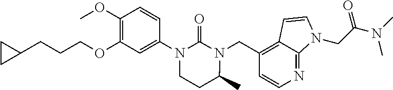 [SMILES: COc1ccc(N2CC[C@H](C)N(Cc3ccnc4c3ccn4CC(=O)N(C)C)C2=O)cc1OCCCC1CC1] | 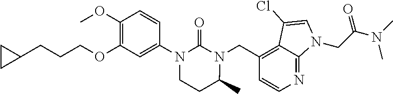 [SMILES: COc1ccc(N2CC[C@H](C)N(Cc3ccnc4c3c(Cl)cn4CC(=O)N(C)C)C2=O)cc1OCCCC1CC1] |
| 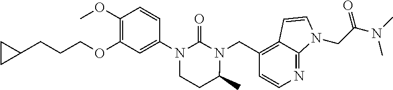 [SMILES: COc1ccc(N2CC[C@H](C)N(Cc3ccnc4c3ccn4CC(=O)N(C)C)C2=O)cc1OCCCC1CC1] | 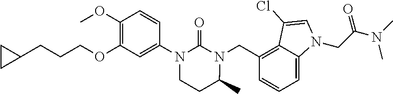 [SMILES: COc1ccc(N2CC[C@H](C)N(Cc3cccc4c3c(Cl)cn4CC(=O)N(C)C)C2=O)cc1OCCCC1CC1] |
| 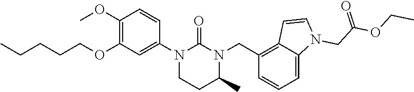 [SMILES: CCCCCOc1cc(N2CC[C@H](C)N(Cc3cccc4c3ccn4CC(=O)OCC)C2=O)ccc1OC] | 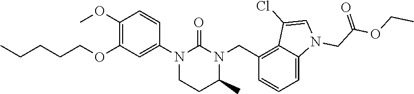 [SMILES: CCCCCOc1cc(N2CC[C@H](C)N(Cc3cccc4c3c(Cl)cn4CC(=O)OCC)C2=O)ccc1OC] |
| 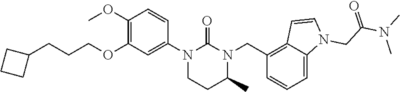 [SMILES: COc1ccc(N2CC[C@H](C)N(Cc3cccc4c3ccn4CC(=O)N(C)C)C2=O)cc1OCCCC1CCC1] | 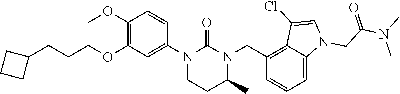 [SMILES: COc1ccc(N2CC[C@H](C)N(Cc3cccc4c3c(Cl)cn4CC(=O)N(C)C)C2=O)cc1OCCCC1CCC1] |
| 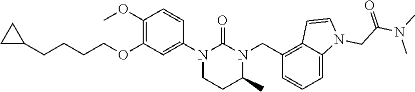 [SMILES: COc1ccc(N2CC[C@H](C)N(Cc3cccc4c3ccn4CC(=O)N(C)C)C2=O)cc1OCCCCC1CC1] | 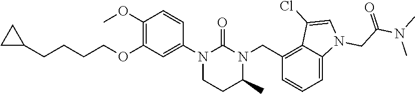 [SMILES: COc1ccc(N2CC[C@H](C)N(Cc3cccc4c3c(Cl)cn4CC(=O)N(C)C)C2=O)cc1OCCCCC1CC1] |
| 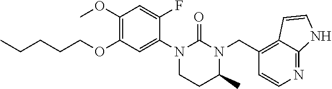 [SMILES: CCCCCOc1cc(N2CC[C@H](C)N(Cc3ccnc4[nH]ccc34)C2=O)c(F)cc1OC] | 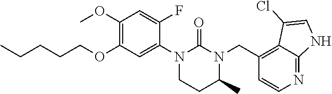 [SMILES: CCCCCOc1cc(N2CC[C@H](C)N(Cc3ccnc4[nH]cc(Cl)c34)C2=O)c(F)cc1OC] |

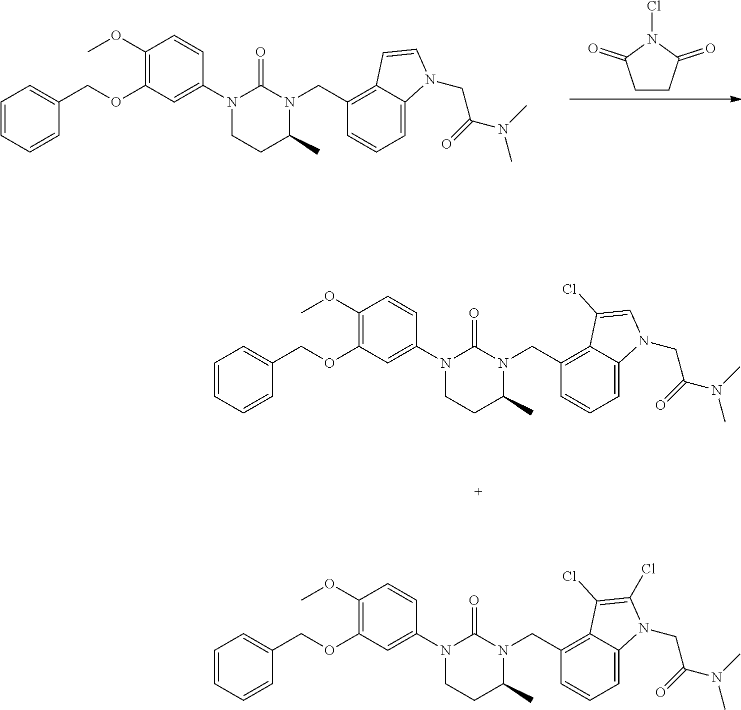 [SMILES: COc1ccc(N2CC[C@H](C)N(Cc3cccc4c3ccn4CC(=O)N(C)C)C2=O)cc1OCc1ccccc1 ; O=C1CCC(=O)N1Cl ; COc1ccc(N2CC[C@H](C)N(Cc3cccc4c3c(Cl)cn4CC(=O)N(C)C)C2=O)cc1OCc1ccccc1 ; COc1ccc(N2CC[C@H](C)N(Cc3cccc4c3c(Cl)c(Cl)n4CC(=O)N(C)C)C2=O)cc1OCc1ccccc1]

To a solution of (S)-2-(4-((3-(3-(benzyloxy)-4-methoxyphenyl)-6-methyl-2-oxotetrahydropyrimidin-1(2H)-yl)methyl)-1H-indol-1-yl)-N,N-dimethylacetamide (220 mg, 407 μmol) in tetrahydrofuran (1.36 mL) at 0° C. was added N-chlorosuccinimide (60.4 mg, 448 μmol). The solution is allowed to warm to room temperature for 14 h before concentration in vacuo. The crude compound is purified by prep-HPLC to give (S)-2-(4-((3-(3-(benzyloxy)-4-methoxyphenyl)-6-methyl-2-oxotetrahydropyrimidin-1(2H)-yl)methyl)-3-chloro-1H-indol-1-yl)-N,N-dimethylacetamide (80 mg, 34% yield) as a white solid and (S)-2-(4-((3-(3-(benzyloxy)-4-methoxyphenyl)-6-methyl-2-oxotetrahydropyrimidin-1(2H)-yl)methyl)-2,3-dichloro-1H-indol-1-yl)-N,N-dimethylacetamide (42 mg, 17% yield) as a white solid.

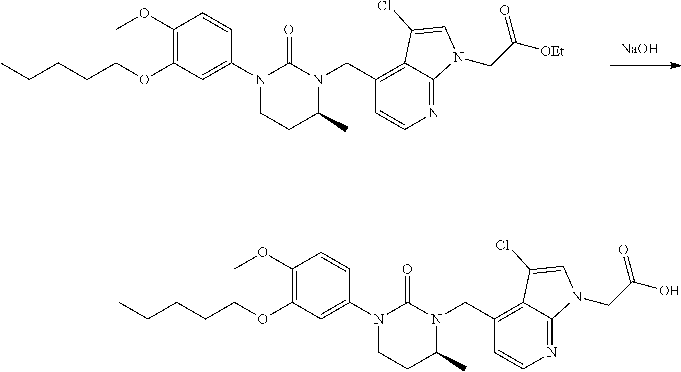 [SMILES: CCCCCOc1cc(N2CC[C@H](C)N(Cc3ccnc4c3c(Cl)cn4CC(=O)OCC)C2=O)ccc1OC ; "NaOH" ; CCCCCOc1cc(N2CC[C@H](C)N(Cc3ccnc4c3c(Cl)cn4CC(=O)O)C2=O)ccc1OC]

Ethyl (S)-2-(3-chloro-4-((3-(4-methoxy-3-(pentyloxy)phenyl)-6-methyl-2-oxotetrahydropyrimidin-1(2H)-yl)methyl)-1H-pyrrolo[2,3-b]pyridin-1-yl)acetate (0.85 g, 1.5 mmol) is dissolved in tetrahydrofuran (3.8 mL). The solution is cooled to 0° C. before addition of 2 N aqueous sodium hydroxide solution (3.8 mL, 7.6 mmol). The reaction mixture is stirred at room temperature for 3 h before cooled to 0° C. before neutralized with 2 N HCl aqueous solution. The mixture is extracted with chloroform/isopropanol (v/v 3:1) three times. The combined organic layers are dried over anhydrous sodium sulfate, filtered and concentrated in vacuo to give (S)-2-(3-chloro-4-((3-(4-methoxy-3-(pentyloxy)phenyl)-6-methyl-2-oxotetrahydropyrimidin-1(2H)-yl)methyl)-1H-pyrrolo[2,3-b]pyridin-1-yl)acetic acid (0.7 g, 87% yield) as a white solid.

The following compounds are prepared in a similar manner as (S)-2-(3-chloro-4-((3-(4-methoxy-3-(pentyloxy)phenyl)-6-methyl-2-oxotetrahydropyrimidin-1(2H)-yl)methyl)-1H-pyrrolo[2,3-b]pyridin-1-yl)acetic acid, as described above.

| Substrate | Compound |
|---|---|
| 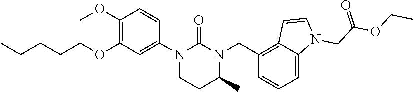 [SMILES: CCCCCOc1cc(N2CC[C@H](C)N(Cc3cccc4c3ccn4CC(=O)OCC)C2=O)ccc1OC] | 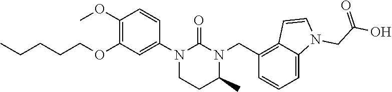 [SMILES: CCCCCOc1cc(N2CC[C@H](C)N(Cc3cccc4c3ccn4CC(=O)O)C2=O)ccc1OC] |
| 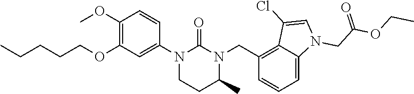 [SMILES: CCCCCOc1cc(N2CC[C@H](C)N(Cc3cccc4c3c(Cl)cn4CC(=O)OCC)C2=O)ccc1OC] | 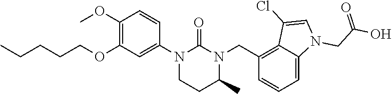 [SMILES: CCCCCOc1cc(N2CC[C@H](C)N(Cc3cccc4c3c(Cl)cn4CC(=O)O)C2=O)ccc1OC] |
| 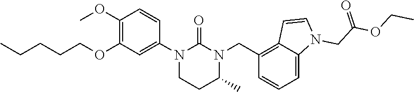 [SMILES: CCCCCOc1cc(N2CC[C@@H](C)N(Cc3cccc4c3ccn4CC(=O)OCC)C2=O)ccc1OC] | 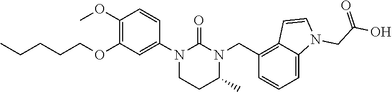 [SMILES: CCCCCOc1cc(N2CC[C@@H](C)N(Cc3cccc4c3ccn4CC(=O)O)C2=O)ccc1OC] |
| 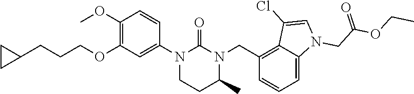 [SMILES: CCOC(=O)Cn1cc(Cl)c2c(CN3C(=O)N(c4ccc(OC)c(OCCCC5CC5)c4)CC[C@@H]3C)cccc21] | 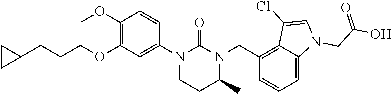 [SMILES: COc1ccc(N2CC[C@H](C)N(Cc3cccc4c3c(Cl)cn4CC(=O)O)C2=O)cc1OCCCC1CC1] |
| 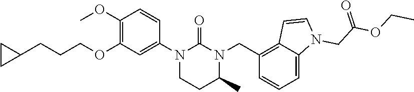 [SMILES: CCOC(=O)Cn1ccc2c(CN3C(=O)N(c4ccc(OC)c(OCCCC5CC5)c4)CC[C@@H]3C)cccc21] | 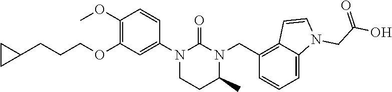 [SMILES: COc1ccc(N2CC[C@H](C)N(Cc3cccc4c3ccn4CC(=O)O)C2=O)cc1OCCCC1CC1] |

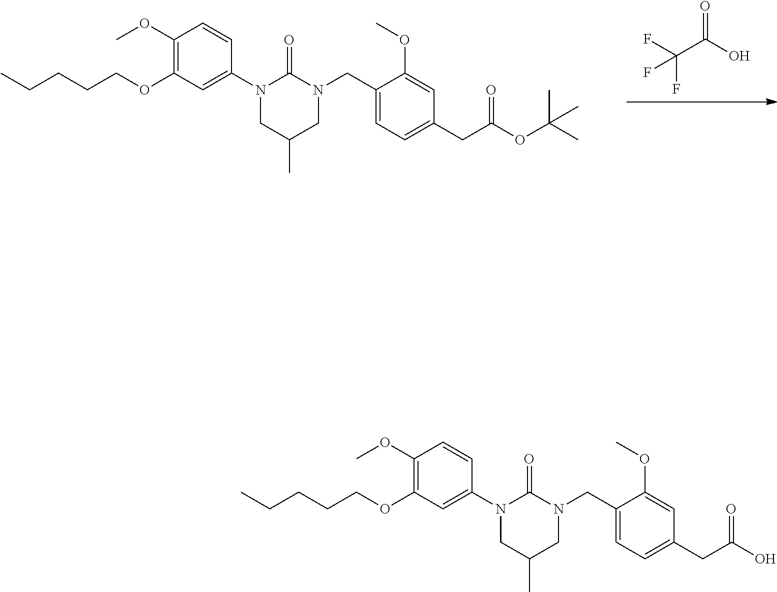

Trifluoroacetic acid (5.1 mL) was added to tert-butyl 2-[4-[[(4-methoxy-3-pentoxyphenyl)carbamoylamino]methyl]pyrrolo[2,3-b]pyridin-1-yl]acetate (433 mg, 0.80 mmol) at 0° C. The reaction mixture is stirred at 0° C. for 10 min, then warmed to room temperature and stirred for 2 h. The volatiles are removed in vacuo. Diethyl ether is added to the residue, and the resulting suspension was sonicated for 2 min. The ether is decanted, and the remaining solids are further triturated with methanol to afford 2-(3-methoxy-4-((3-(4-methoxy-3-(pentyloxy)phenyl)-5-methyl-2-oxotetrahydropyrimidin-1(2H)-yl)methyl)phenyl)acetic acid (109 mg, 28% yield) as a white solid.

The following compounds are prepared in a similar manner as 2-(3-methoxy-4-((3-(4-methoxy-3-(pentyloxy)phenyl)-5-methyl-2-oxotetrahydropyrimidin-1(2H)-yl)methyl)phenyl)acetic acid, as described above.

| Substrate | Compound |
|---|---|
| 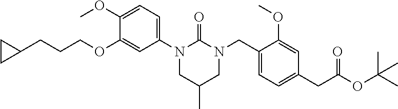 [SMILES: COc1cc(CC(=O)OC(C)(C)C)ccc1CN1CC(C)CN(c2ccc(OC)c(OCCCC3CC3)c2)C1=O] | 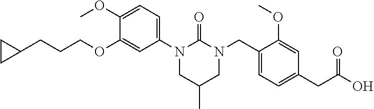 [SMILES: COc1cc(CC(=O)O)ccc1CN1CC(C)CN(c2ccc(OC)c(OCCCC3CC3)c2)C1=O] |
| 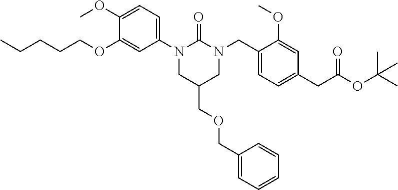 [SMILES: CCCCCOc1cc(N2CC(COCc3ccccc3)CN(Cc3ccc(CC(=O)OC(C)(C)C)cc3OC)C2=O)ccc1OC] | 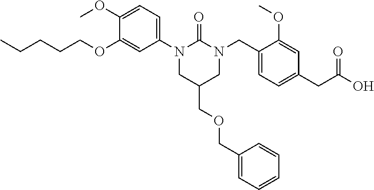 [SMILES: CCCCCOc1cc(N2CC(COCc3ccccc3)CN(Cc3ccc(CC(=O)O)cc3OC)C2=O)ccc1OC] |

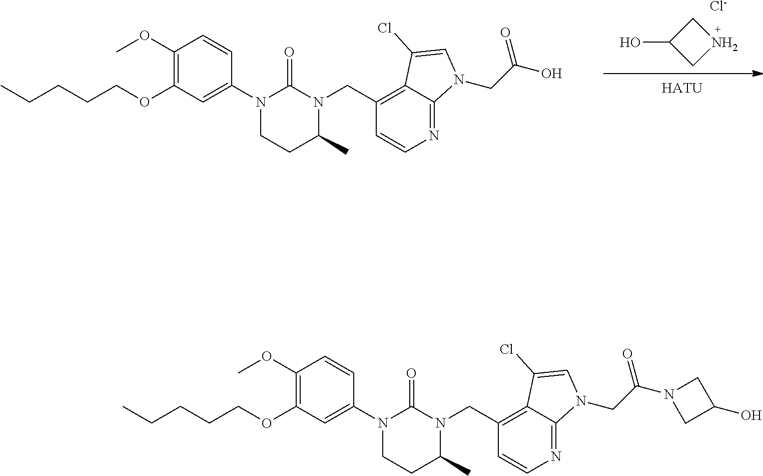

(S)-2-(3-chloro-4-((3-(4-methoxy-3-(pentyloxy)phenyl)-6-methyl-2-oxotetrahydropyrimidin-1(2H)-yl)methyl)-1H-pyrrolo[2,3-b]pyridin-1-yl)acetic acid (63 mg, 0.10 mmol) is dissolved in N,N-dimethylformamide (1.2 mL), then HATU (1-[bis(dimethylamino)methylene]-1H-1,2,3-triazolo[4,5-b]pyridinium 3-oxid hexafluorophosphate, 40 mg, 0.11 mmol), 3-hydroxyazetidine hydrochloride (12 mg, 0.11 mmol), and N,N-diisopropylethylamine (0.04 mL, 0.22 mmol) are added. Reaction mixture is stirred at room temperature for 2 h. The crude is purified by prep-HPLC to afford (S)-3-((3-chloro-1-(2-(3-hydroxyazetidin-1-yl)-2-oxoethyl)-1H-pyrrolo[2,3-b]pyridin-4-yl)methyl)-1-(4-methoxy-3-(pentyloxy)phenyl)-4-methyltetrahydropyrimidin-2(1H)-one.

The following compounds are prepared in a similar manner as (S)-3-((3-chloro-1-(2-(3-hydroxyazetidin-1l-yl)-2-oxoethyl)-1H-pyrrolo[2,3-b]pyridin-4-yl)methyl)-1-(4-methoxy-3-(pentyloxy)phenyl)-4-methyltetrahydropyrimidin-2(1H)-one, as described above.

| Acid | Amine | Compound |
|---|---|---|
| 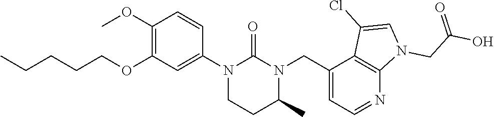 [SMILES: CCCCCOc1cc(N2CC[C@H](C)N(Cc3ccnc4c3c(Cl)cn4CC(=O)O)C2=O)ccc1OC] | 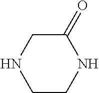 | 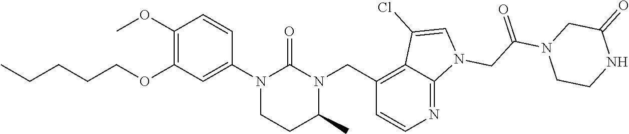 [SMILES: CCCCCOc1cc(N2CC[C@H](C)N(Cc3ccnc4c3c(Cl)cn4CC(=O)N3CCNC(=O)C3)C2=O)ccc1OC] |
| 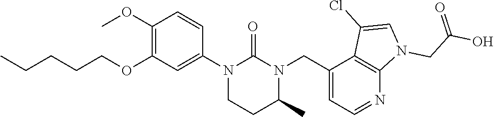 [SMILES: CCCCCOc1cc(N2CC[C@H](C)N(Cc3ccnc4c3c(Cl)cn4CC(=O)O)C2=O)ccc1OC] | 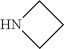 | 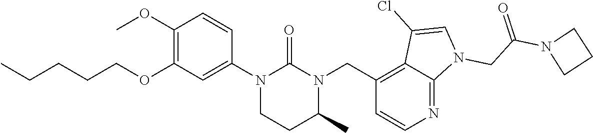 [SMILES: CCCCCOc1cc(N2CC[C@H](C)N(Cc3ccnc4c3c(Cl)cn4CC(=O)N3CCC3)C2=O)ccc1OC] |
| 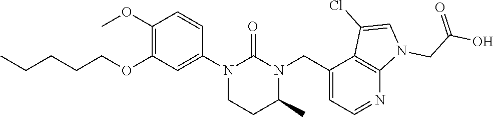 [SMILES: CCCCCOc1cc(N2CC[C@H](C)N(Cc3ccnc4c3c(Cl)cn4CC(=O)O)C2=O)ccc1OC] | 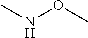 | 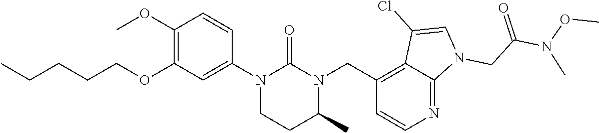 [SMILES: CCCCCOc1cc(N2CC[C@H](C)N(Cc3ccnc4c3c(Cl)cn4CC(=O)N(C)OC)C2=O)ccc1OC] |
| 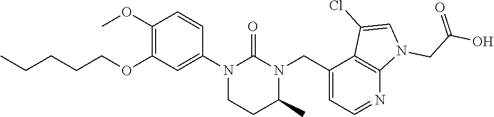 [SMILES: CCCCCOc1cc(N2CC[C@H](C)N(Cc3ccnc4c3c(Cl)cn4CC(=O)O)C2=O)ccc1OC] | 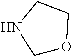 | 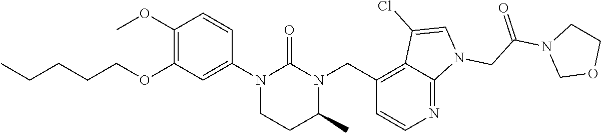 [SMILES: CCCCCOc1cc(N2CC[C@H](C)N(Cc3ccnc4c3c(Cl)cn4CC(=O)N3CCOC3)C2=O)ccc1OC] |
| 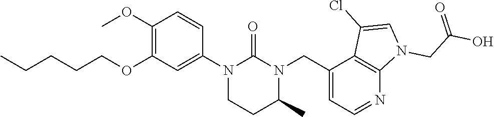 [SMILES: CCCCCOc1cc(N2CC[C@H](C)N(Cc3ccnc4c3c(Cl)cn4CC(=O)O)C2=O)ccc1OC] | 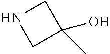 | 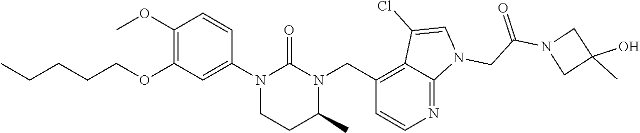 [SMILES: CCCCCOc1cc(N2CC[C@H](C)N(Cc3ccnc4c3c(Cl)cn4CC(=O)N3CC(C)(O)C3)C2=O)ccc1OC] |
| 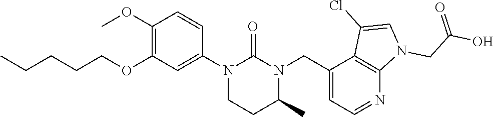 [SMILES: CCCCCOc1cc(N2CC[C@H](C)N(Cc3ccnc4c3c(Cl)cn4CC(=O)O)C2=O)ccc1OC] | 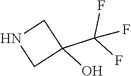 | 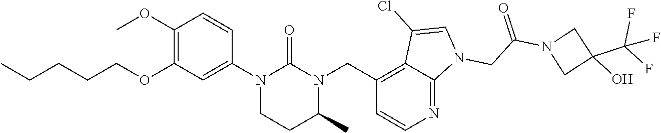 [SMILES: CCCCCOc1cc(N2CC[C@H](C)N(Cc3ccnc4c3c(Cl)cn4CC(=O)N3CC(O)(C(F)(F)F)C3)C2=O)ccc1OC] |
| 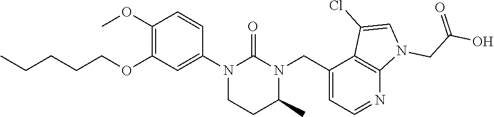 [SMILES: CCCCCOc1cc(N2CC[C@H](C)N(Cc3ccnc4c3c(Cl)cn4CC(=O)O)C2=O)ccc1OC] | 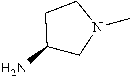 | 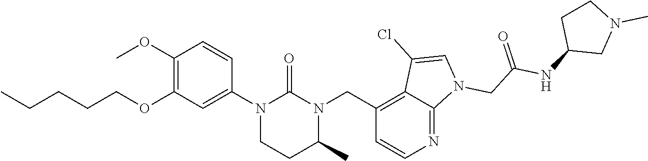 [SMILES: CCCCCOc1cc(N2CC[C@H](C)N(Cc3ccnc4c3c(Cl)cn4CC(=O)N[C@H]3CCN(C)C3)C2=O)ccc1OC] |
| 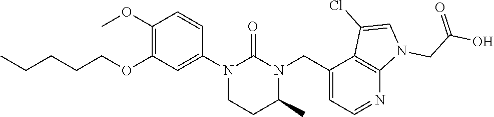 [SMILES: CCCCCOc1cc(N2CC[C@H](C)N(Cc3ccnc4c3c(Cl)cn4CC(=O)O)C2=O)ccc1OC] | 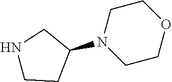 [SMILES: C1C[C@H](N2CCOCC2)CN1] | 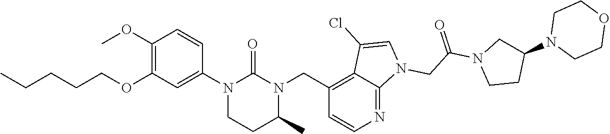 [SMILES: CCCCCOc1cc(N2CC[C@H](C)N(Cc3ccnc4c3c(Cl)cn4CC(=O)N3CC[C@H](N4CCOCC4)C3)C2=O)ccc1OC] |
| 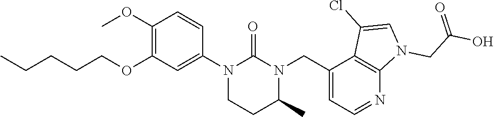 [SMILES: CCCCCOc1cc(N2CC[C@H](C)N(Cc3ccnc4c3c(Cl)cn4CC(=O)O)C2=O)ccc1OC] | 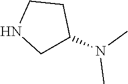 | 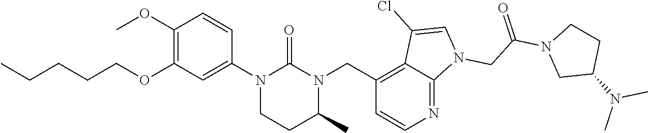 [SMILES: CCCCCOc1cc(N2CC[C@H](C)N(Cc3ccnc4c3c(Cl)cn4CC(=O)N3CC[C@H](N(C)C)C3)C2=O)ccc1OC] |
| 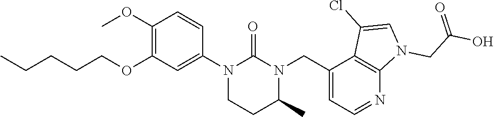 [SMILES: CCCCCOc1cc(N2CC[C@H](C)N(Cc3ccnc4c3c(Cl)cn4CC(=O)O)C2=O)ccc1OC] | 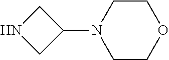 | 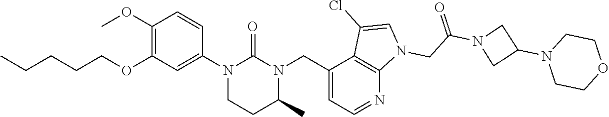 [SMILES: CCCCCOc1cc(N2CC[C@H](C)N(Cc3ccnc4c3c(Cl)cn4CC(=O)N3CC(N4CCOCC4)C3)C2=O)ccc1OC] |
| 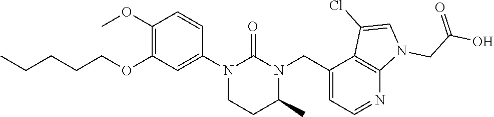 [SMILES: CCCCCOc1cc(N2CC[C@H](C)N(Cc3ccnc4c3c(Cl)cn4CC(=O)O)C2=O)ccc1OC] | 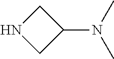 | 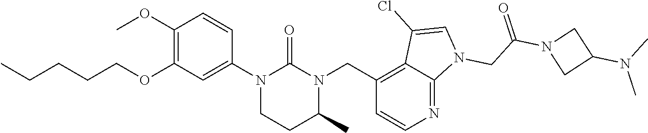 [SMILES: CCCCCOc1cc(N2CC[C@H](C)N(Cc3ccnc4c3c(Cl)cn4CC(=O)N3CC(N(C)C)C3)C2=O)ccc1OC] |
| 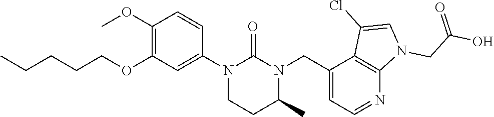 [SMILES: CCCCCOc1cc(N2CC[C@H](C)N(Cc3ccnc4c3c(Cl)cn4CC(=O)O)C2=O)ccc1OC] | 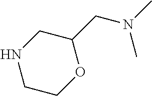 | 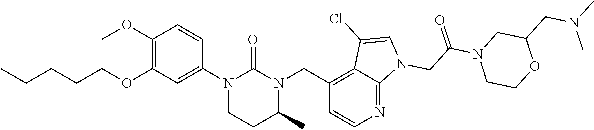 [SMILES: CCCCCOc1cc(N2CC[C@H](C)N(Cc3ccnc4c3c(Cl)cn4CC(=O)N3CCOC(CN(C)C)C3)C2=O)ccc1OC] |
| 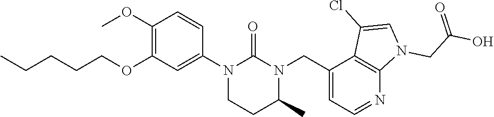 [SMILES: CCCCCOc1cc(N2CC[C@H](C)N(Cc3ccnc4c3c(Cl)cn4CC(=O)O)C2=O)ccc1OC] | 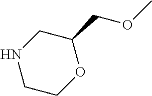 | 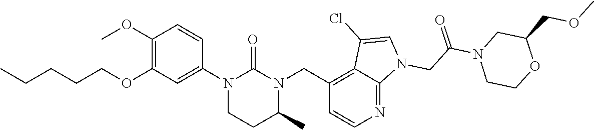 [SMILES: CCCCCOc1cc(N2CC[C@H](C)N(Cc3ccnc4c3c(Cl)cn4CC(=O)N3CCO[C@H](COC)C3)C2=O)ccc1OC] |
| 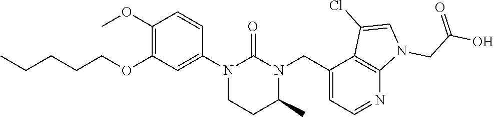 [SMILES: CCCCCOc1cc(N2CC[C@H](C)N(Cc3ccnc4c3c(Cl)cn4CC(=O)O)C2=O)ccc1OC] | 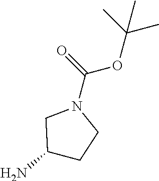 | 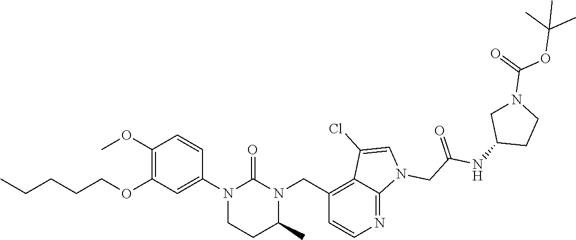 [SMILES: CCCCCOc1cc(N2CC[C@H](C)N(Cc3ccnc4c3c(Cl)cn4CC(=O)N[C@H]3CCN(C(=O)OC(C)(C)C)C3)C2=O)ccc1OC] |
| 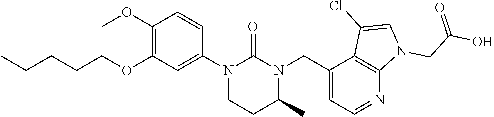 [SMILES: CCCCCOc1cc(N2CC[C@H](C)N(Cc3ccnc4c3c(Cl)cn4CC(=O)O)C2=O)ccc1OC] | 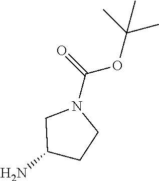 | 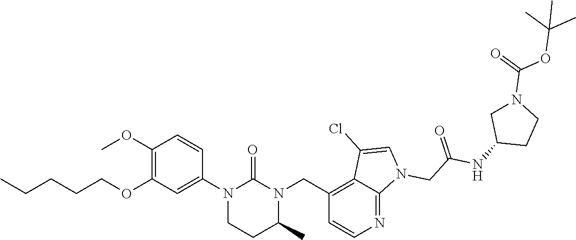 [SMILES: CCCCCOc1cc(N2CC[C@H](C)N(Cc3ccnc4c3c(Cl)cn4CC(=O)N[C@H]3CCN(C(=O)OC(C)(C)C)C3)C2=O)ccc1OC] |
| 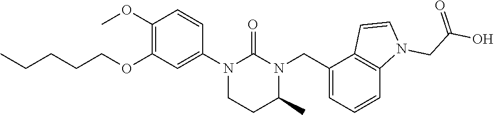 [SMILES: CCCCCOc1cc(N2CC[C@H](C)N(Cc3cccc4c3ccn4CC(=O)O)C2=O)ccc1OC] | 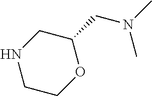 | 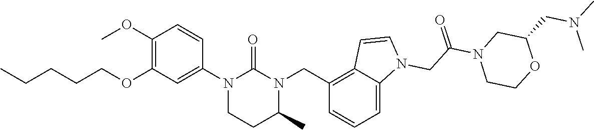 [SMILES: CCCCCOc1cc(N2CC[C@H](C)N(Cc3cccc4c3ccn4CC(=O)N3CCO[C@@H](CN(C)C)C3)C2=O)ccc1OC] |
| 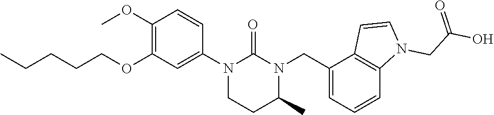 [SMILES: CCCCCOc1cc(N2CC[C@H](C)N(Cc3cccc4c3ccn4CC(=O)O)C2=O)ccc1OC] | 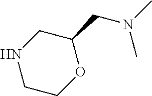 | 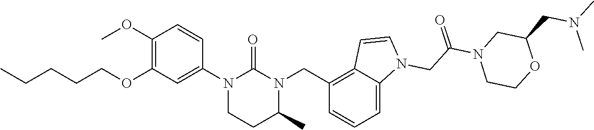 [SMILES: CCCCCOc1cc(N2CC[C@H](C)N(Cc3cccc4c3ccn4CC(=O)N3CCO[C@H](CN(C)C)C3)C2=O)ccc1OC] |
| 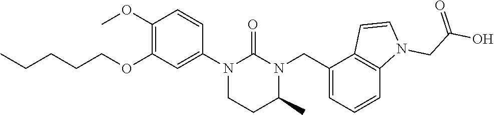 [SMILES: CCCCCOc1cc(N2CC[C@H](C)N(Cc3cccc4c3ccn4CC(=O)O)C2=O)ccc1OC] | 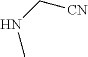 | 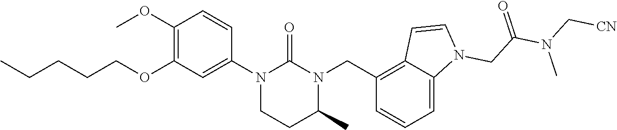 [SMILES: CCCCCOc1cc(N2CC[C@H](C)N(Cc3cccc4c3ccn4CC(=O)N(C)CC#N)C2=O)ccc1OC] |
| 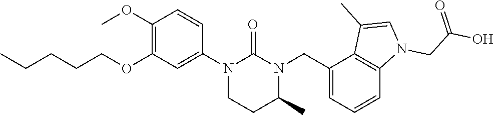 [SMILES: CCCCCOc1cc(N2CC[C@H](C)N(Cc3cccc4c3c(C)cn4CC(=O)O)C2=O)ccc1OC] | 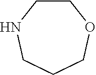 | 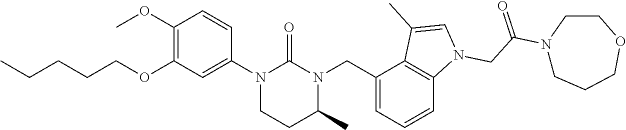 [SMILES: CCCCCOc1cc(N2CC[C@H](C)N(Cc3cccc4c3c(C)cn4CC(=O)N3CCCOCC3)C2=O)ccc1OC] |
| 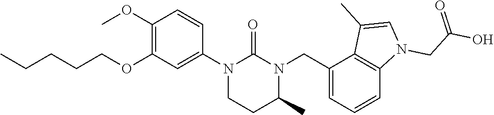 [SMILES: CCCCCOc1cc(N2CC[C@H](C)N(Cc3cccc4c3c(C)cn4CC(=O)O)C2=O)ccc1OC] | 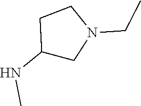 | 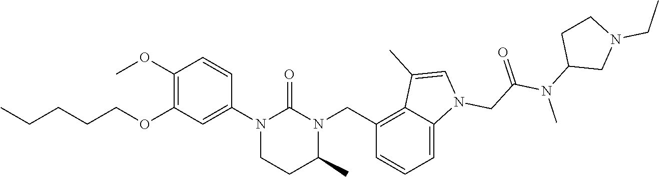 [SMILES: CCCCCOc1cc(N2CC[C@H](C)N(Cc3cccc4c3c(C)cn4CC(=O)N(C)C3CCN(CC)C3)C2=O)ccc1OC] |
| | | Mixed diastereomers |
| 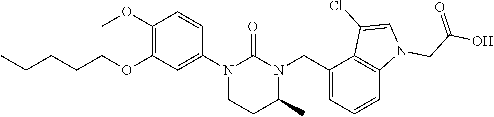 [SMILES: CCCCCOc1cc(N2CC[C@H](C)N(Cc3cccc4c3c(Cl)cn4CC(=O)O)C2=O)ccc1OC] | 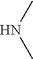 | 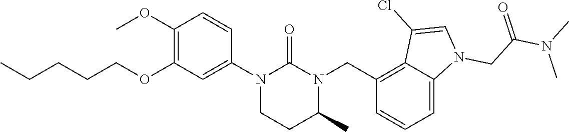 [SMILES: CCCCCOc1cc(N2CC[C@H](C)N(Cc3cccc4c3c(Cl)cn4CC(=O)N(C)C)C2=O)ccc1OC] |
| 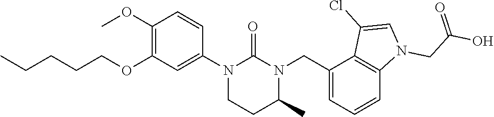 [SMILES: CCCCCOc1cc(N2CC[C@H](C)N(Cc3cccc4c3c(Cl)cn4CC(=O)O)C2=O)ccc1OC] | 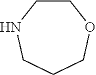 | 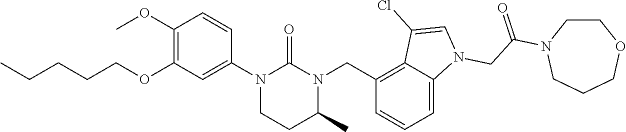 [SMILES: CCCCCOc1cc(N2CC[C@H](C)N(Cc3cccc4c3c(Cl)cn4CC(=O)N3CCCOCC3)C2=O)ccc1OC] |
| 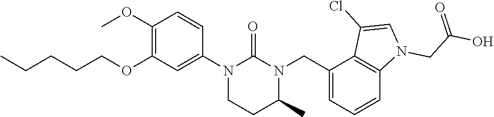 [SMILES: CCCCCOc1cc(N2CC[C@H](C)N(Cc3cccc4c3c(Cl)cn4CC(=O)O)C2=O)ccc1OC] | 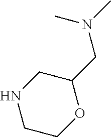 | 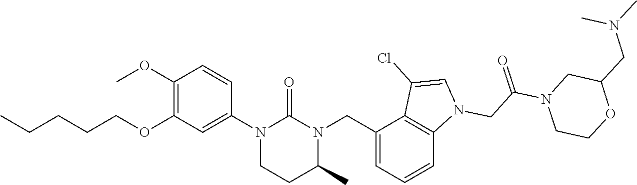 [SMILES: CCCCCOc1cc(N2CC[C@H](C)N(Cc3cccc4c3c(Cl)cn4CC(=O)N3CCOC(CN(C)C)C3)C2=O)ccc1OC] |
| | | Mixed diastereomers |
| 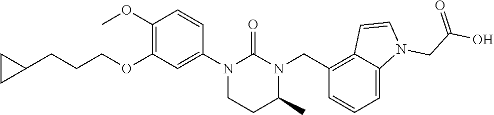 [SMILES: COc1ccc(N2CC[C@H](C)N(Cc3cccc4c3ccn4CC(=O)O)C2=O)cc1OCCCC1CC1] | 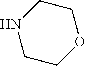 | 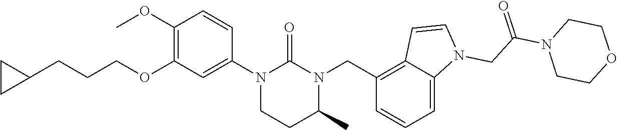 [SMILES: COc1ccc(N2CC[C@H](C)N(Cc3cccc4c3ccn4CC(=O)N3CCOCC3)C2=O)cc1OCCCC1CC1] |

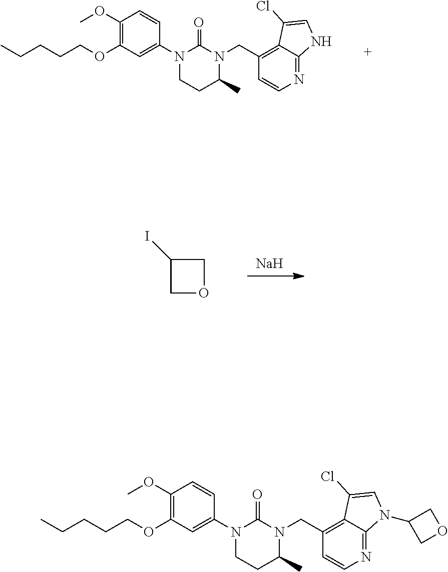

To (S)-3-((3-chloro-1H-pyrrolo[2,3-b]pyridin-4-yl)methyl)-1-(4-methoxy-3-(pentyloxy)phenyl)-4-methyltetrahydropyrimidin-2(1H)-one (50 mg, 0.11 mmol) in tetrahydrofuran (5 mL) is added sodium hydride (60% in mineral oil, 5 mg, 0.13 mmol). After 10 min, 3-iodooxetane (20 mg, 0.11 mmol) is added. The reaction mixture is stirred at room temperature for 14 h. The reaction is cooled to 0° C. and quenched with water. The mixture is extracted with ethyl acetate three times. Combined organic phases are washed with brine, dried over anhydrous sodium sulfate, filtered and concentrated in vacuo. The residue is purified by prep-HPLC to give (S)-3-((3-chloro-1-(oxetan-3-yl)-1H-pyrrolo[2,3-b]pyridin-4-yl)methyl)-1-(4-methoxy-3-(pentyloxy)phenyl)-4-methyltetrahydropyrimidin-2(1H)-one as a yellow solid.

The following compounds are prepared in a similar manner as give (S)-3-((3-chloro-1-(oxetan-3-yl)-1H-pyrrolo[2,3-b]pyridin-4-yl)methyl)-1-(4-methoxy-3-(pentyloxy)phenyl-4-methyltetrahydropyrimidin-2(1H)-one, as described above.

| Substrate | Halide | Compound |
|---|---|---|
| 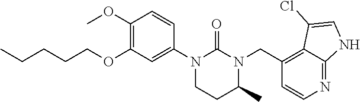 [SMILES: CCCCCOc1cc(N2CC[C@H](C)N(Cc3ccnc4[nH]cc(Cl)c34)C2=O)ccc1OC] | 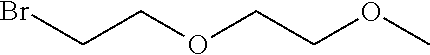 | 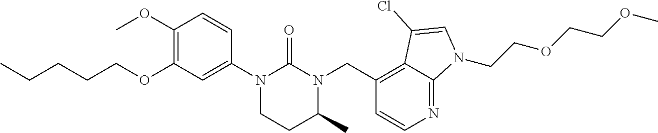 [SMILES: CCCCCOc1cc(N2CC[C@H](C)N(Cc3ccnc4c3c(Cl)cn4CCOCCOC)C2=O)ccc1OC] |
| 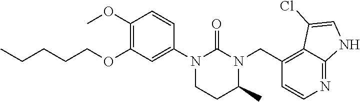 [SMILES: CCCCCOc1cc(N2CC[C@H](C)N(Cc3ccnc4[nH]cc(Cl)c34)C2=O)ccc1OC] | 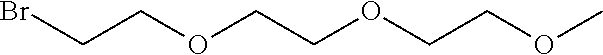 | 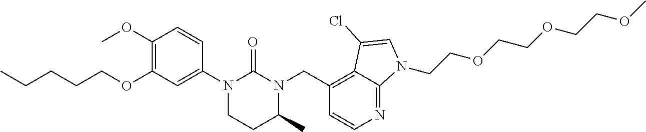 [SMILES: CCCCCOc1cc(N2CC[C@H](C)N(Cc3ccnc4c3c(Cl)cn4CCOCCOCCOC)C2=O)ccc1OC] |
| 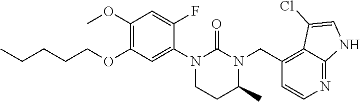 [SMILES: CCCCCOc1cc(N2CC[C@H](C)N(Cc3ccnc4[nH]cc(Cl)c34)C2=O)c(F)cc1OC] | 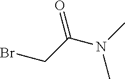 | 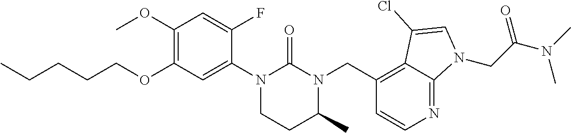 [SMILES: CCCCCOc1cc(N2CC[C@H](C)N(Cc3ccnc4c3c(Cl)cn4CC(=O)N(C)C)C2=O)c(F)cc1OC] |

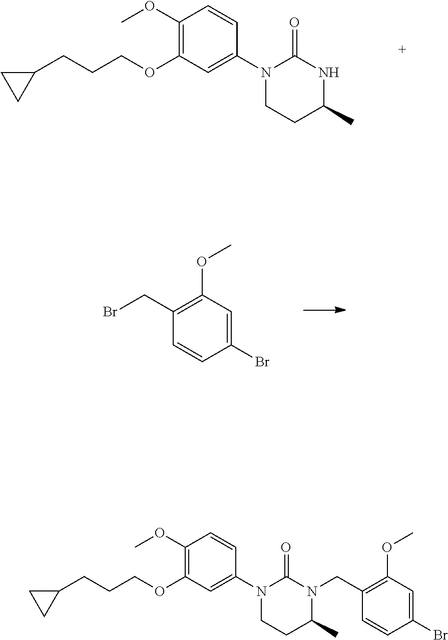 [SMILES: COc1ccc(N2CC[C@H](C)NC2=O)cc1OCCCC1CC1 ; COc1cc(Br)ccc1CBr ; COc1cc(Br)ccc1CN1C(=O)N(c2ccc(OC)c(OCCCC3CC3)c2)CC[C@@H]1C]

To sodium hydride (60% of dispersion in mineral oil, 0.58 g, 14.62 mmol) in N,N-dimethylformamide (15 mL) at 0° C. is added (S)-1-(3-(3-cyclopropylpropoxy)-4-methoxyphenyl)-4-methyltetrahydropyrimidin-2(1H)-one (1.55 g, 4.87 mmol) in N,N-dimethylformamide (15 mL). The reaction mixture is stirred at 0° C. for 30 min before addition of 4-bromo-1-(bromomethyl)-2-methoxybenzene (1.50 g, 5.36 mmol) in N,N-dimethylformamide (12 mL). The resulting mixture is stirred at room temperature for 2 h before cooled to 0° C. and quenched by saturated ammonium chloride aqueous solution. The aqueous mixture is extracted with dichloromethane three times and combined organic layers were washed with brine, dried over anhydrous sodium sulfate, filtered and concentrated in vacuo. Crude (S)-3-(4-bromo-2-methoxybenzyl)-1-(3-(3-cyclopropylpropoxy)-4-methoxyphenyl)-4-methyltetrahydropyrimidin-2(1H)-one is used for next step without further purification.

The following compounds are prepared in a similar manner as (S)-3-(4-bromo-2-methoxybenzyl)-1-(3-(3-cyclopropylpropoxy)-4-methoxyphenyl)-4-methyltetrahydropyrimidin-2(1H)-one, as described above.

| Substrate | Bromide | Compound |
|---|---|---|
| 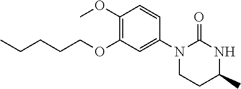 [SMILES: CCCCCOc1cc(N2CC[C@H](C)NC2=O)ccc1OC] | 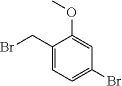 | 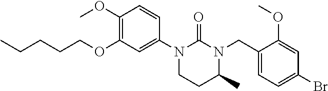 [SMILES: CCCCCOc1cc(N2CC[C@H](C)N(Cc3ccc(Br)cc3OC)C2=O)ccc1OC] |
| 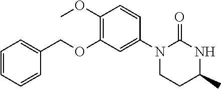 [SMILES: COc1ccc(N2CC[C@H](C)NC2=O)cc1OCc1ccccc1] | 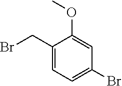 | 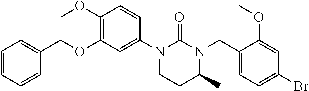 [SMILES: COc1cc(Br)ccc1CN1C(=O)N(c2ccc(OC)c(OCc3ccccc3)c2)CC[C@@H]1C] |

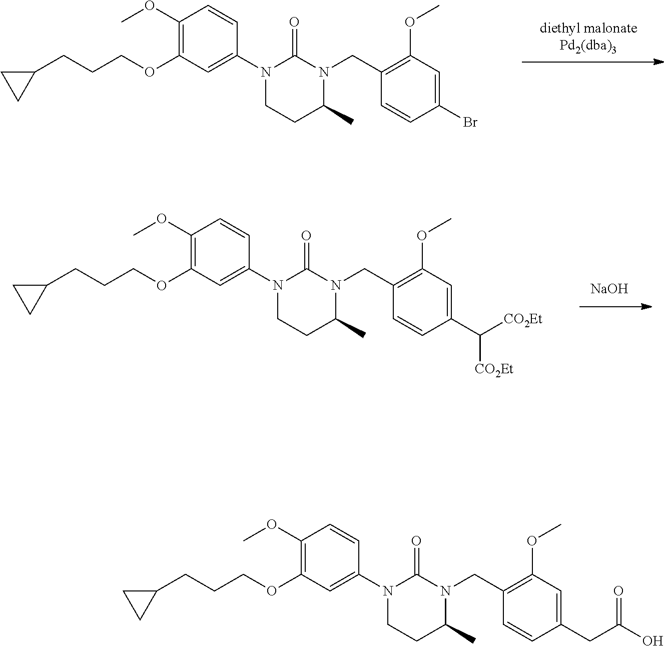 [SMILES: COc1cc(Br)ccc1CN1C(=O)N(c2ccc(OC)c(OCCCC3CC3)c2)CC[C@@H]1C ; CCOC(=O)C(C(=O)OCC)c1ccc(CN2C(=O)N(c3ccc(OC)c(OCCCC4CC4)c3)CC[C@@H]2C)c(OC)c1 ; "NaOH" ; COc1cc(CC(=O)O)ccc1CN1C(=O)N(c2ccc(OC)c(OCCCC3CC3)c2)CC[C@@H]1C]

Cesium carbonate (16.9 g, 68 mmol) is added to a solution of (S)-3-(4-bromo-2-methoxybenzyl)-1-(3-(3-cyclopropylpropoxy)-4-methoxyphenyl)-4-methyltetrahydropyrimidin-2(1H)-one (17.6 g, 34 mmol) and diethyl malonate (10.9 g, 68 mmol) in N,N-dimethylformamide (150 mL). The mixture is degassed with a flow of nitrogen for 5 min, then Pd₂(dba)₃ (1.0 g, 1.09 mmol) and 2-dicyclohexylphosphino-2′,6′-dimethoxybiphenyl (1.0 g, 2.44 mmol) are added. The mixture is heated under 95° C. for 12 h. After cooled to room temperature, the mixture is quenched with water and extracted with ethyl acetate. The combined organic phases are washed with brine and dried over anhydrous sodium sulfate, filtered and concentrated in vacuo. The residue is purified by flash chromatography over silica gel (hexane/ethyl acetate, v/v 5:1 to 3:1) to afford diethyl (S)-2-(4-((3-(3-(3-cyclopropylpropoxy)-4-methoxyphenyl)-6-methyl-2-oxotetrahydropyrimidin-1(21)-yl)methyl)-3-methoxyphenyl)malonate (14.2 g, 70% yield) as an oil.

A solution of diethyl (S)-2-(4-((3-(3-(3-cyclopropylpropoxy)-4-methoxyphenyl)-6-methyl-2-oxotetrahydropyrimidin-1(2H)-yl)methyl)-3-methoxyphenyl)malonate (14.2 g, 24 mmol) and NaOH (2.0 g, 50 mmol) in a mixture of ethanol/water (300 mL, v/v 1:2) is heated under reflux for 12 h. After cooled to room temperature, the mixture is washed with ethyl acetate/hexane (v/v 1:1, 200 mL×3), then the aqueous layer is acidified to pH=3 with 1 N hydrochloric acid aqueous solution. The resulting mixture is heated under reflux for 2 h. After cooled to room temperature again, the mixture is extracted with ethyl acetate. The combined organic phases are washed with brine and dried over anhydrous sodium sulfate, filtered and concentrated to afford (S)-2-(4-((3-(3-(3-cyclopropylpropoxy)-4-methoxyphenyl)-6-methyl-2-oxotetrahydropyrimidin-1(2H)-yl)methyl)-3-methoxyphenyl)acetic acid (9.6 g, 81% yield) as an oil, which is used for next step without further purification.

The following compounds are prepared in a similar manner as (S)-2-(4-((3-(3-(3-cyclopropylpropoxy)-4-methoxyphenyl)-6-methyl-2-oxotetrahydropyrimidin-1(2H)-yl)methyl)-3-methoxyphenyl)acetic acid, as described above.

| Substrate | Compound |
|---|---|
| 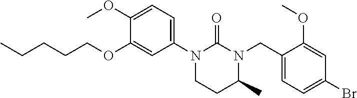 [SMILES: CCCCCOc1cc(N2CC[C@H](C)N(Cc3ccc(Br)cc3OC)C2=O)ccc1OC] | 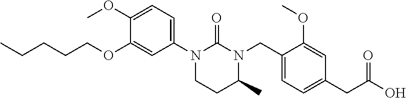 [SMILES: CCCCCOc1cc(N2CC[C@H](C)N(Cc3ccc(CC(=O)O)cc3OC)C2=O)ccc1OC] |

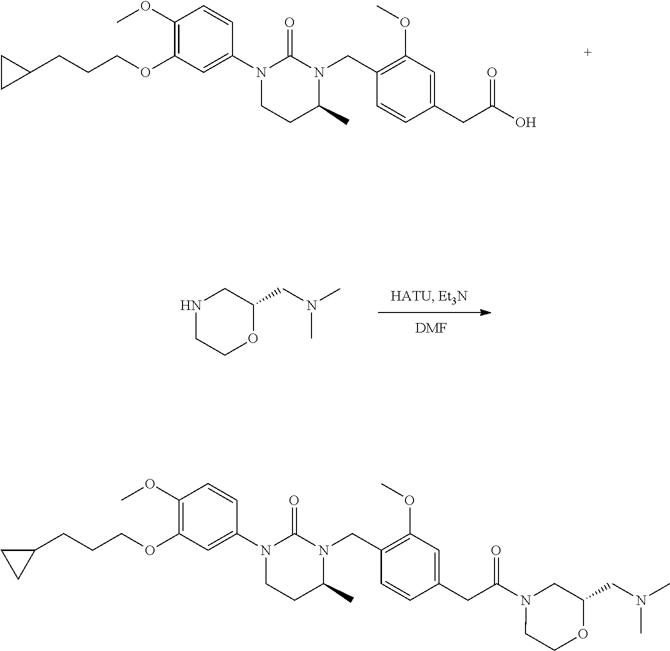

To a round bottom flask are added (S)-2-(4-((3-(3-(3-cyclopropylpropoxy)-4-methoxyphenyl)-6-methyl-2-oxotetrahydropyrimidin-1(2H)-yl)methyl)-3-methoxyphenyl)acetic acid (0.5 g, 1.01 mmol), (R)—N,N-dimethyl-1-(morpholin-2-yl)methanamine (146 mg, 1.01 mmol) and N,N-dimethylformamide (6 mL). The mixture is cooled to 0° C. before addition of HATU (0.42 g, 1.10 mmol) and triethylamine (0.21 mL, 1.51 mmol). The reaction mixture is stirred at room temperature for 16 h. The reaction mixture is purified by reverse phase chromatography to obtain (S)-1-(3-(3-cyclopropylpropoxy)-4-methoxyphenyl)-3-(4-(2-((S)-2-((dimethylamino)methyl)morpholino)-2-oxoethyl)-2-methoxybenzyl)-4-methyltetrahydropyrimidin-2(1H)-one (0.52 g, 83% yield) as a white solid.

The following compounds are prepared in a similar manner as (S)-1-(3-(3-cyclopropylpropoxy)-4-methoxyphenyl)-3-(4-(2-((S)-2-((dimethylamino)methyl)morpholino)-2-oxoethyl)-2-methoxybenzyl)-4-methyltetrahydropyrimidin-2(1H)one, as described above.

| Carboxylic acid | Reactant | Compound |
|---|---|---|
| 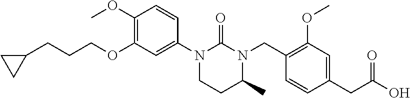 [SMILES: COc1cc(CC(=O)O)ccc1CN1C(=O)N(c2ccc(OC)c(OCCCC3CC3)c2)CC[C@@H]1C] | 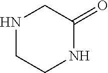 | 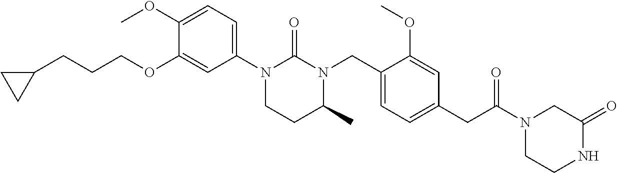 [SMILES: COc1cc(CC(=O)N2CCNC(=O)C2)ccc1CN1C(=O)N(c2ccc(OC)c(OCCCC3CC3)c2)CC[C@@H]1C] |
| 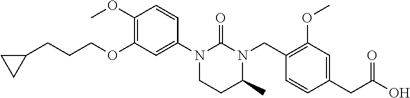 [SMILES: COc1cc(CC(=O)O)ccc1CN1C(=O)N(c2ccc(OC)c(OCCCC3CC3)c2)CC[C@@H]1C] | 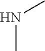 | 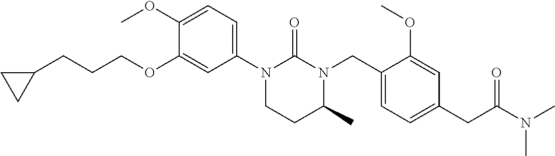 [SMILES: COc1cc(CC(=O)N(C)C)ccc1CN1C(=O)N(c2ccc(OC)c(OCCCC3CC3)c2)CC[C@@H]1C] |
| 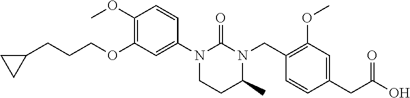 [SMILES: COc1cc(CC(=O)O)ccc1CN1C(=O)N(c2ccc(OC)c(OCCCC3CC3)c2)CC[C@@H]1C] | 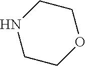 | 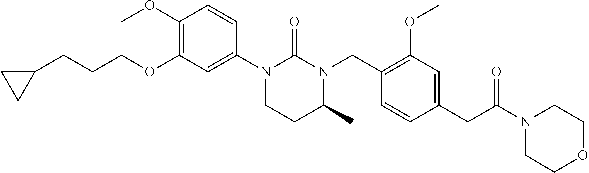 [SMILES: COc1cc(CC(=O)N2CCOCC2)ccc1CN1C(=O)N(c2ccc(OC)c(OCCCC3CC3)c2)CC[C@@H]1C] |
| 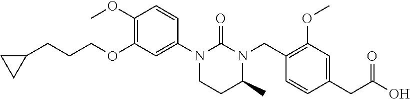 [SMILES: COc1cc(CC(=O)O)ccc1CN1C(=O)N(c2ccc(OC)c(OCCCC3CC3)c2)CC[C@@H]1C] | 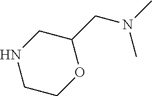 | 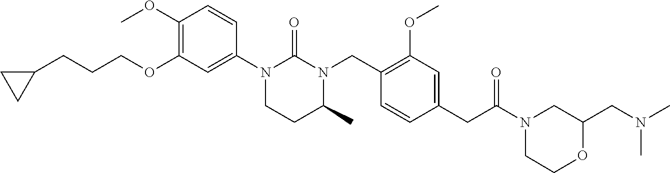 [SMILES: COc1cc(CC(=O)N2CCOC(CN(C)C)C2)ccc1CN1C(=O)N(c2ccc(OC)c(OCCCC3CC3)c2)CC[C@@H]1C] |
| | | Mixed diastereomers |
| 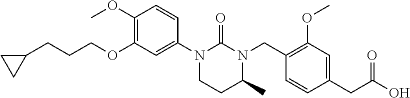 [SMILES: COc1cc(CC(=O)O)ccc1CN1C(=O)N(c2ccc(OC)c(OCCCC3CC3)c2)CC[C@@H]1C] | 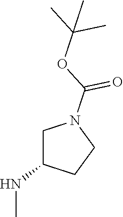 | 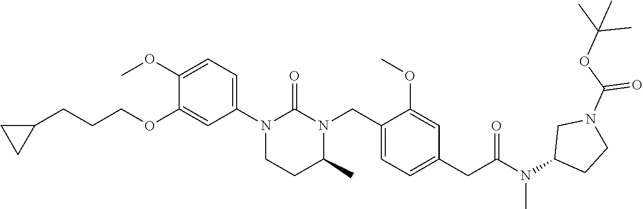 [SMILES: COc1cc(CC(=O)N(C)[C@H]2CCN(C(=O)OC(C)(C)C)C2)ccc1CN1C(=O)N(c2ccc(OC)c(OCCCC3CC3)c2)CC[C@@H]1C] |
| 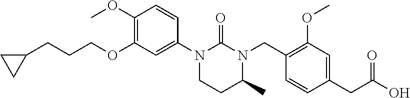 [SMILES: COc1cc(CC(=O)O)ccc1CN1C(=O)N(c2ccc(OC)c(OCCCC3CC3)c2)CC[C@@H]1C] | 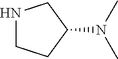 | 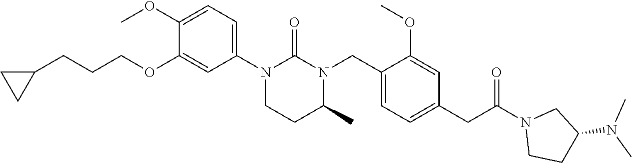 [SMILES: COc1cc(CC(=O)N2CC[C@@H](N(C)C)C2)ccc1CN1C(=O)N(c2ccc(OC)c(OCCCC3CC3)c2)CC[C@@H]1C] |
| 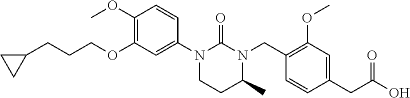 [SMILES: COc1cc(CC(=O)O)ccc1CN1C(=O)N(c2ccc(OC)c(OCCCC3CC3)c2)CC[C@@H]1C] | 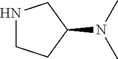 | 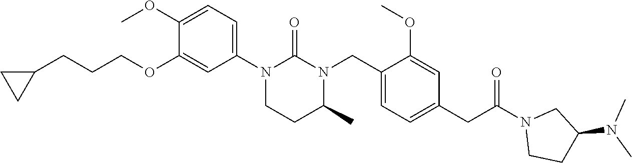 [SMILES: COc1cc(CC(=O)N2CC[C@H](N(C)C)C2)ccc1CN1C(=O)N(c2ccc(OC)c(OCCCC3CC3)c2)CC[C@@H]1C] |
| 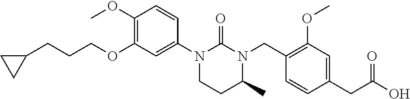 [SMILES: COc1cc(CC(=O)O)ccc1CN1C(=O)N(c2ccc(OC)c(OCCCC3CC3)c2)CC[C@@H]1C] | 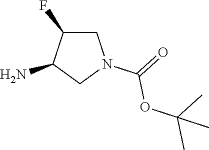 | 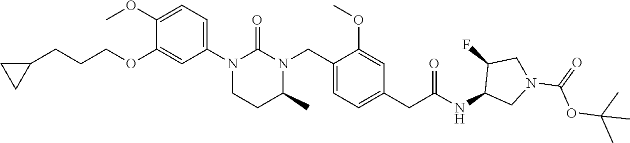 [SMILES: COc1cc(CC(=O)N[C@@H]2CN(C(=O)OC(C)(C)C)C[C@@H]2F)ccc1CN1C(=O)N(c2ccc(OC)c(OCCCC3CC3)c2)CC[C@@H]1C] |
| 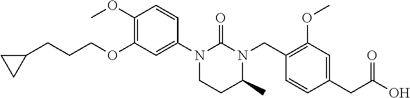 [SMILES: COc1cc(CC(=O)O)ccc1CN1C(=O)N(c2ccc(OC)c(OCCCC3CC3)c2)CC[C@@H]1C] | 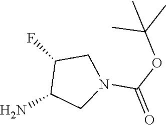 | 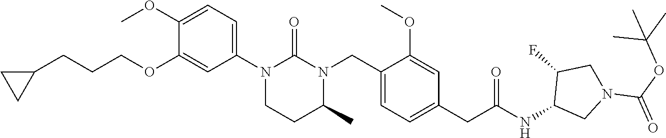 [SMILES: COc1cc(CC(=O)N[C@H]2CN(C(=O)OC(C)(C)C)C[C@H]2F)ccc1CN1C(=O)N(c2ccc(OC)c(OCCCC3CC3)c2)CC[C@@H]1C] |
| 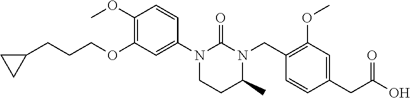 [SMILES: COc1cc(CC(=O)O)ccc1CN1C(=O)N(c2ccc(OC)c(OCCCC3CC3)c2)CC[C@@H]1C] | 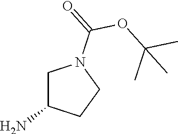 | 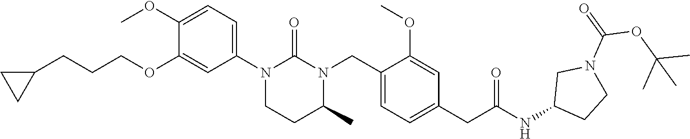 [SMILES: COc1cc(CC(=O)N[C@H]2CCN(C(=O)OC(C)(C)C)C2)ccc1CN1C(=O)N(c2ccc(OC)c(OCCCC3CC3)c2)CC[C@@H]1C] |
| 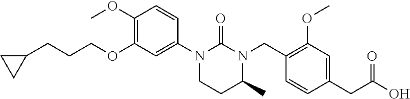 [SMILES: COc1cc(CC(=O)O)ccc1CN1C(=O)N(c2ccc(OC)c(OCCCC3CC3)c2)CC[C@@H]1C] | 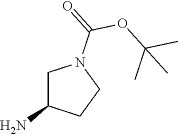 | 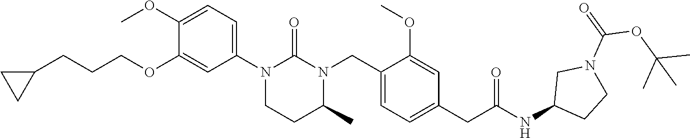 [SMILES: COc1cc(CC(=O)N[C@@H]2CCN(C(=O)OC(C)(C)C)C2)ccc1CN1C(=O)N(c2ccc(OC)c(OCCCC3CC3)c2)CC[C@@H]1C] |
| 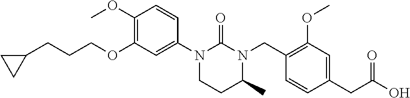 [SMILES: COc1cc(CC(=O)O)ccc1CN1C(=O)N(c2ccc(OC)c(OCCCC3CC3)c2)CC[C@@H]1C] | 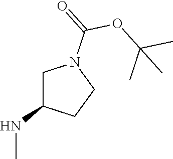 | 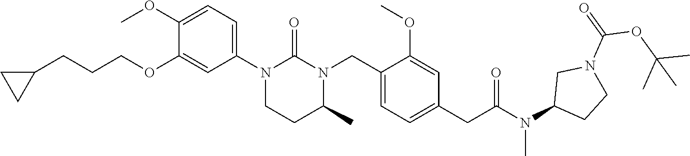 [SMILES: COc1cc(CC(=O)N(C)[C@@H]2CCN(C(=O)OC(C)(C)C)C2)ccc1CN1C(=O)N(c2ccc(OC)c(OCCCC3CC3)c2)CC[C@@H]1C] |
| 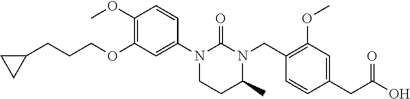 [SMILES: COc1cc(CC(=O)O)ccc1CN1C(=O)N(c2ccc(OC)c(OCCCC3CC3)c2)CC[C@@H]1C] | 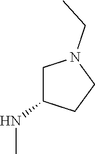 | 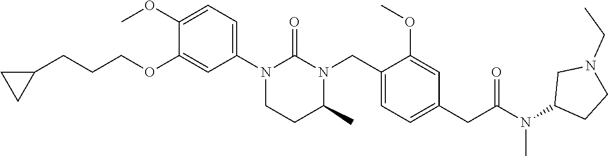 |
| 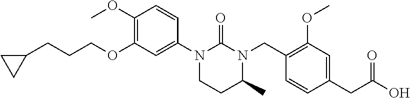 [SMILES: COc1cc(CC(=O)O)ccc1CN1C(=O)N(c2ccc(OC)c(OCCCC3CC3)c2)CC[C@@H]1C] | 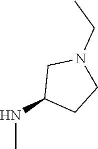 | 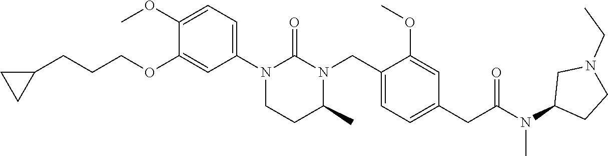 [SMILES: CCN1CC[C@@H](N(C)C(=O)Cc2ccc(CN3C(=O)N(c4ccc(OC)c(OCCCC5CC5)c4)CC[C@@H]3C)c(OC)c2)C1] |
| 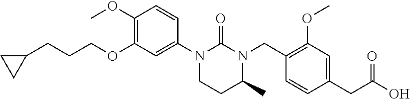 [SMILES: COc1cc(CC(=O)O)ccc1CN1C(=O)N(c2ccc(OC)c(OCCCC3CC3)c2)CC[C@@H]1C] | 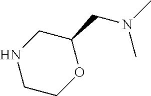 | 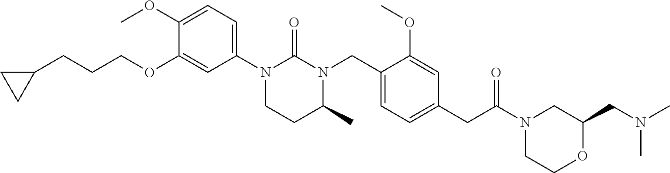 [SMILES: COc1cc(CC(=O)N2CCO[C@H](CN(C)C)C2)ccc1CN1C(=O)N(c2ccc(OC)c(OCCCC3CC3)c2)CC[C@@H]1C] |
| 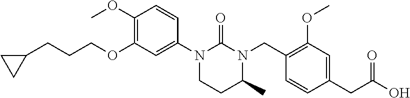 [SMILES: COc1cc(CC(=O)O)ccc1CN1C(=O)N(c2ccc(OC)c(OCCCC3CC3)c2)CC[C@@H]1C] | 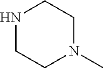 | 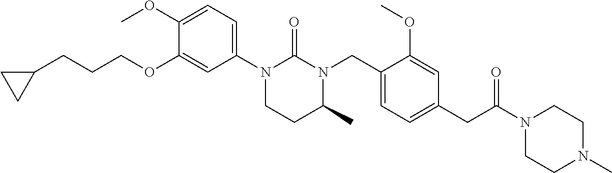 [SMILES: COc1cc(CC(=O)N2CCN(C)CC2)ccc1CN1C(=O)N(c2ccc(OC)c(OCCCC3CC3)c2)CC[C@@H]1C] |
| 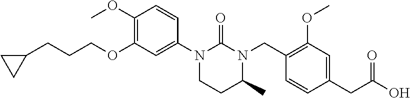 [SMILES: COc1cc(CC(=O)O)ccc1CN1C(=O)N(c2ccc(OC)c(OCCCC3CC3)c2)CC[C@@H]1C] | 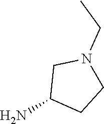 | 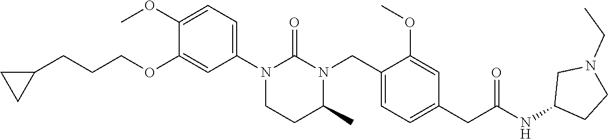 [SMILES: CCN1CC[C@H](NC(=O)Cc2ccc(CN3C(=O)N(c4ccc(OC)c(OCCCC5CC5)c4)CC[C@@H]3C)c(OC)c2)C1] |
| 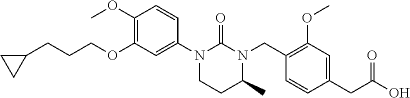 [SMILES: COc1cc(CC(=O)O)ccc1CN1C(=O)N(c2ccc(OC)c(OCCCC3CC3)c2)CC[C@@H]1C] | 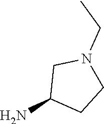 | 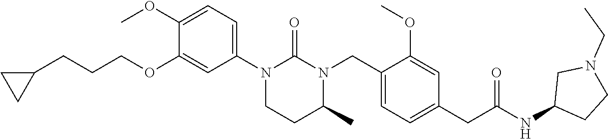 [SMILES: CCN1CC[C@@H](NC(=O)Cc2ccc(CN3C(=O)N(c4ccc(OC)c(OCCCC5CC5)c4)CC[C@@H]3C)c(OC)c2)C1] |
| 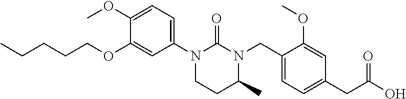 [SMILES: CCCCCOc1cc(N2CC[C@H](C)N(Cc3ccc(CC(=O)O)cc3OC)C2=O)ccc1OC] | 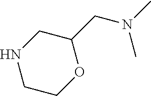 | 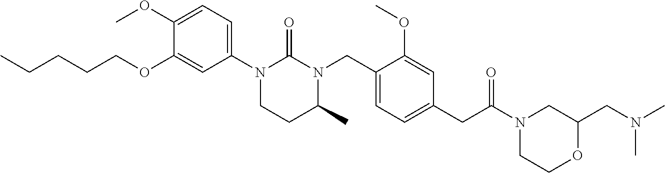 [SMILES: CCCCCOc1cc(N2CC[C@H](C)N(Cc3ccc(CC(=O)N4CCOC(CN(C)C)C4)cc3OC)C2=O)ccc1OC] |
| | | Mixed diastereomers |
| 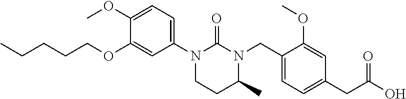 [SMILES: CCCCCOc1cc(N2CC[C@H](C)N(Cc3ccc(CC(=O)O)cc3OC)C2=O)ccc1OC] | 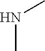 | 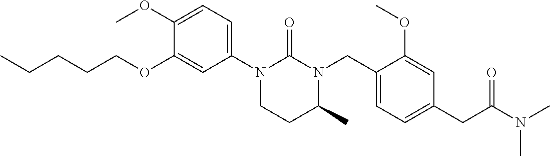 [SMILES: CCCCCOc1cc(N2CC[C@H](C)N(Cc3ccc(CC(=O)N(C)C)cc3OC)C2=O)ccc1OC] |
| 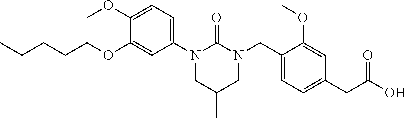 [SMILES: CCCCCOc1cc(N2CC(C)CN(Cc3ccc(CC(=O)O)cc3OC)C2=O)ccc1OC] | 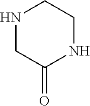 | 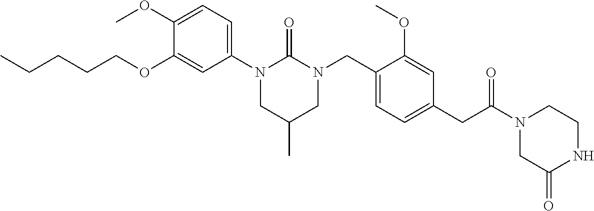 [SMILES: CCCCCOc1cc(N2CC(C)CN(Cc3ccc(CC(=O)N4CCNC(=O)C4)cc3OC)C2=O)ccc1OC] |
| | | racemate |
| 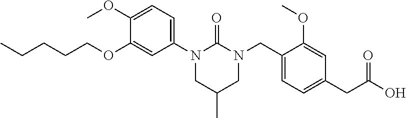 [SMILES: CCCCCOc1cc(N2CC(C)CN(Cc3ccc(CC(=O)O)cc3OC)C2=O)ccc1OC] | 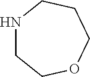 | 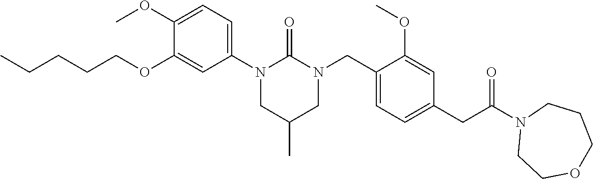 [SMILES: CCCCCOc1cc(N2CC(C)CN(Cc3ccc(CC(=O)N4CCCOCC4)cc3OC)C2=O)ccc1OC] |
| | | Racemate |
| 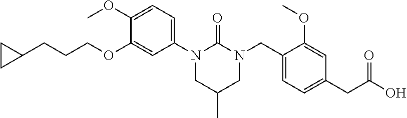 [SMILES: COc1cc(CC(=O)O)ccc1CN1CC(C)CN(c2ccc(OC)c(OCCCC3CC3)c2)C1=O] | 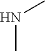 | 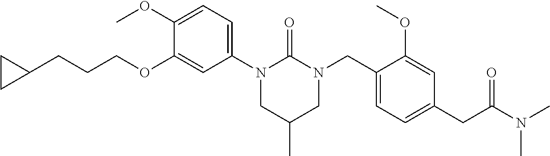 [SMILES: COc1cc(CC(=O)N(C)C)ccc1CN1CC(C)CN(c2ccc(OC)c(OCCCC3CC3)c2)C1=O] |
| | | Racemate |
| 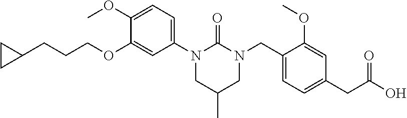 [SMILES: COc1cc(CC(=O)O)ccc1CN1CC(C)CN(c2ccc(OC)c(OCCCC3CC3)c2)C1=O] | 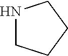 | 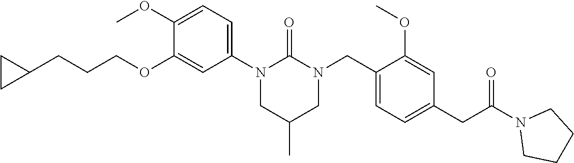 [SMILES: COc1cc(CC(=O)N2CCCC2)ccc1CN1CC(C)CN(c2ccc(OC)c(OCCCC3CC3)c2)C1=O] |
| | | Racemate |
| 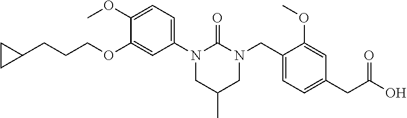 [SMILES: COc1cc(CC(=O)O)ccc1CN1CC(C)CN(c2ccc(OC)c(OCCCC3CC3)c2)C1=O] | 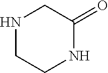 | 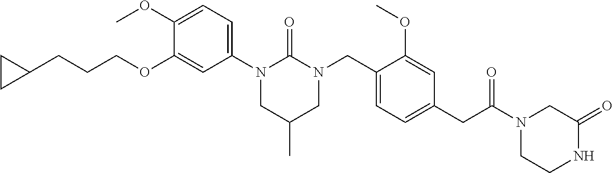 [SMILES: COc1cc(CC(=O)N2CCNC(=O)C2)ccc1CN1CC(C)CN(c2ccc(OC)c(OCCCC3CC3)c2)C1=O] |
| | | Racemate |
| 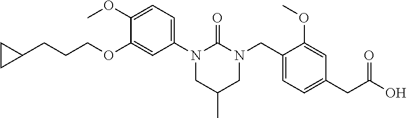 [SMILES: COc1cc(CC(=O)O)ccc1CN1CC(C)CN(c2ccc(OC)c(OCCCC3CC3)c2)C1=O] | NH₃ | 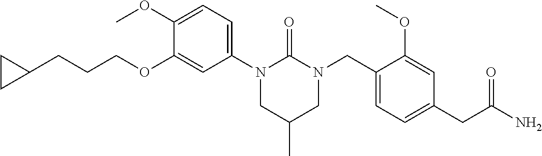 [SMILES: COc1cc(CC(N)=O)ccc1CN1CC(C)CN(c2ccc(OC)c(OCCCC3CC3)c2)C1=O] |
| | | Racemate |
| 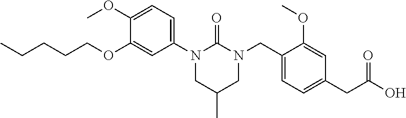 [SMILES: CCCCCOc1cc(N2CC(C)CN(Cc3ccc(CC(=O)O)cc3OC)C2=O)ccc1OC] | 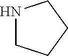 | 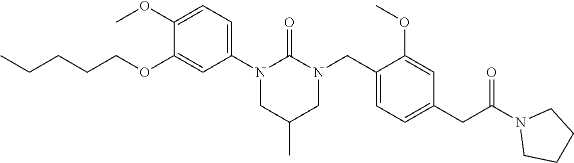 [SMILES: CCCCCOc1cc(N2CC(C)CN(Cc3ccc(CC(=O)N4CCCC4)cc3OC)C2=O)ccc1OC] |
| 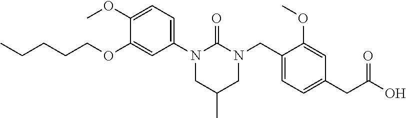 [SMILES: CCCCCOc1cc(N2CC(C)CN(Cc3ccc(CC(=O)O)cc3OC)C2=O)ccc1OC] | 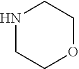 | 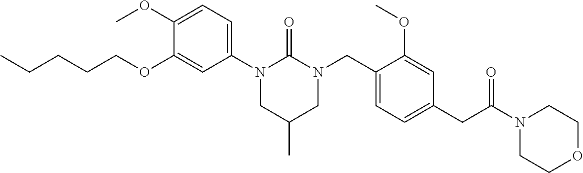 [SMILES: CCCCCOc1cc(N2CC(C)CN(Cc3ccc(CC(=O)N4CCOCC4)cc3OC)C2=O)ccc1OC] |
| 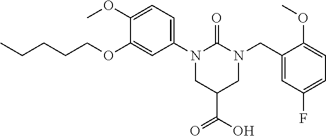 [SMILES: CCCCCOc1cc(N2CC(C(=O)O)CN(Cc3cc(F)ccc3OC)C2=O)ccc1OC] | 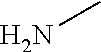 | 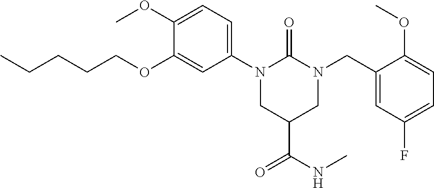 [SMILES: CCCCCOc1cc(N2CC(C(=O)NC)CN(Cc3cc(F)ccc3OC)C2=O)ccc1OC] |
| 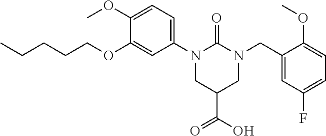 [SMILES: CCCCCOc1cc(N2CC(C(=O)O)CN(Cc3cc(F)ccc3OC)C2=O)ccc1OC] | NH₃ | 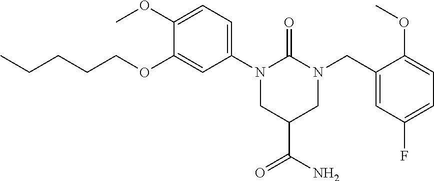 [SMILES: CCCCCOc1cc(N2CC(C(N)=O)CN(Cc3cc(F)ccc3OC)C2=O)ccc1OC] |
| 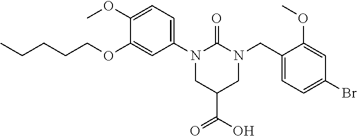 [SMILES: CCCCCOc1cc(N2CC(C(=O)O)CN(Cc3ccc(Br)cc3OC)C2=O)ccc1OC] | NH₃ | 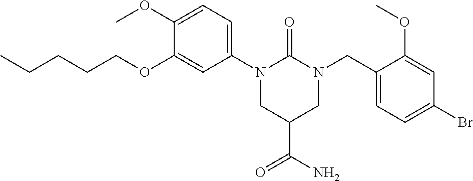 [SMILES: CCCCCOc1cc(N2CC(C(N)=O)CN(Cc3ccc(Br)cc3OC)C2=O)ccc1OC] |
| 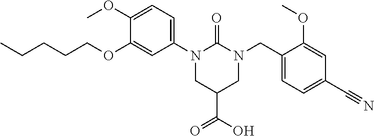 [SMILES: CCCCCOc1cc(N2CC(C(=O)O)CN(Cc3ccc(C#N)cc3OC)C2=O)ccc1OC] | NH₃ | 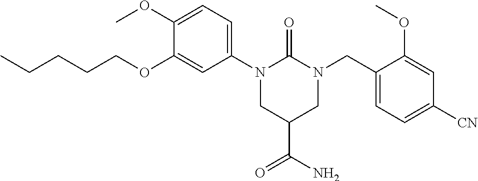 [SMILES: CCCCCOc1cc(N2CC(C(N)=O)CN(Cc3ccc(C#N)cc3OC)C2=O)ccc1OC] |
| 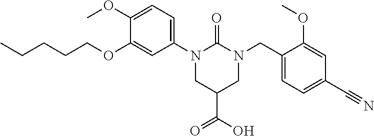 [SMILES: CCCCCOc1cc(N2CC(C(=O)O)CN(Cc3ccc(C#N)cc3OC)C2=O)ccc1OC] | 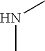 | 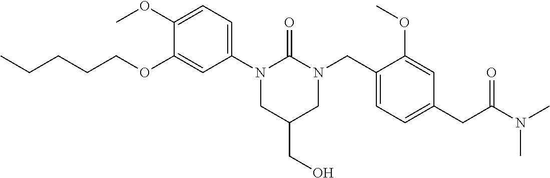 [SMILES: CCCCCOc1cc(N2CC(CO)CN(Cc3ccc(CC(=O)N(C)C)cc3OC)C2=O)ccc1OC] |
| 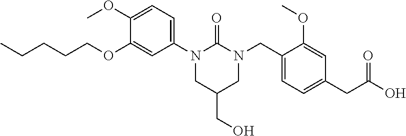 [SMILES: CCCCCOc1cc(N2CC(CO)CN(Cc3ccc(CC(=O)O)cc3OC)C2=O)ccc1OC] | 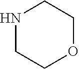 | 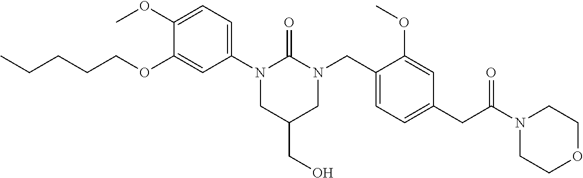 [SMILES: CCCCCOc1cc(N2CC(CO)CN(Cc3ccc(CC(=O)N4CCOCC4)cc3OC)C2=O)ccc1OC] |
| 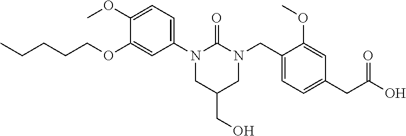 [SMILES: CCCCCOc1cc(N2CC(CO)CN(Cc3ccc(CC(=O)O)cc3OC)C2=O)ccc1OC] | 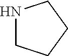 | 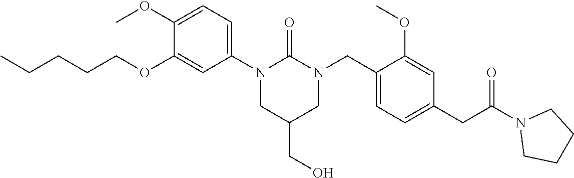 [SMILES: CCCCCOc1cc(N2CC(CO)CN(Cc3ccc(CC(=O)N4CCCC4)cc3OC)C2=O)ccc1OC] |
| 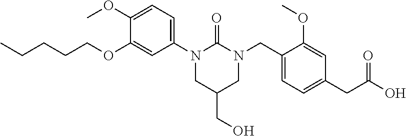 [SMILES: CCCCCOc1cc(N2CC(CO)CN(Cc3ccc(CC(=O)O)cc3OC)C2=O)ccc1OC] | 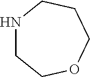 | 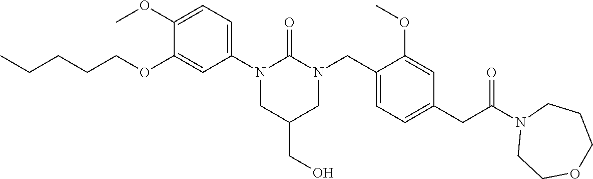 [SMILES: CCCCCOc1cc(N2CC(CO)CN(Cc3ccc(CC(=O)N4CCCOCC4)cc3OC)C2=O)ccc1OC] |
| 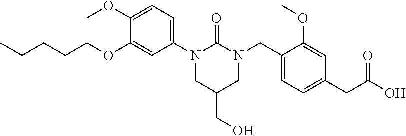 [SMILES: CCCCCOc1cc(N2CC(CO)CN(Cc3ccc(CC(=O)O)cc3OC)C2=O)ccc1OC] | 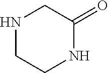 | 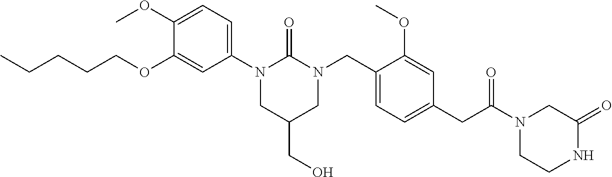 [SMILES: CCCCCOc1cc(N2CC(CO)CN(Cc3ccc(CC(=O)N4CCNC(=O)C4)cc3OC)C2=O)ccc1OC] |

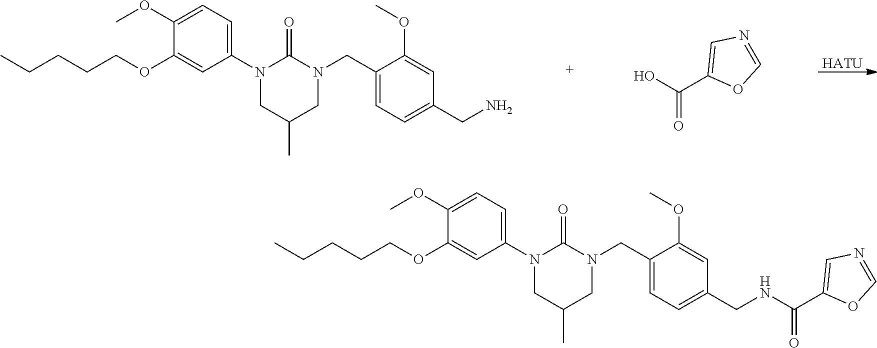

To a round bottom flask are added 1-(4-(aminomethyl)-2-methoxybenzyl)-3-(4-methoxy-3-(pentyloxy)phenyl)-5-methyltetrahydropyrimidin-2(1H)-one (0.23 g, 0.50 mmol oxazole-5-carboxylic acid (68 mg, 0.60 mmol) and N,N-dimethylformamide (3 mL). The mixture is cooled to 0° C. before addition of HATU (0.23 g, 0.60 mmol) and triethylamine (0.08 mL, 0.60 mmol). The reaction mixture is stirred at room temperature for 16 h. The reaction mixture is purified by reverse phase chromatography to afford N-(3-methoxy-4-((3-(4-methoxy-3-(pentyloxy)phenyl)-5-methyl-2-oxotetrahydropyrimidin-1(2K)-yl)methyl)benzyl)oxazole-5-carboxamide (0.22 g, 80% yield) as a white solid.

The following compounds are prepared in a similar manner as N-(3-methoxy-4-((3-(4-methoxy-3-(pentyloxy)phenyl)-5-methyl-2-oxotetrahydropyrimidin-1(2H)-yl)methyl)benzyl) oxazole-5-carboxamide, as described above.

| | Carboxylic | |
|---|---|---|
| Amine | acid | Compound |
| 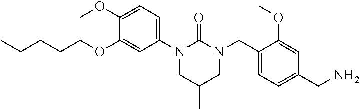 [SMILES: CCCCCOc1cc(N2CC(C)CN(Cc3ccc(CN)cc3OC)C2=O)ccc1OC] | 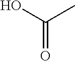 | 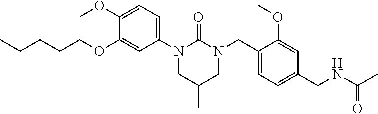 [SMILES: CCCCCOc1cc(N2CC(C)CN(Cc3ccc(CNC(C)=O)cc3OC)C2=O)ccc1OC] |
| 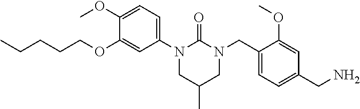 [SMILES: CCCCCOc1cc(N2CC(C)CN(Cc3ccc(CN)cc3OC)C2=O)ccc1OC] | 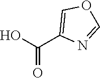 | 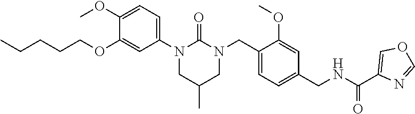 [SMILES: CCCCCOc1cc(N2CC(C)CN(Cc3ccc(CNC(=O)c4cocn4)cc3OC)C2=O)ccc1OC] |
| | | Racemate |
| 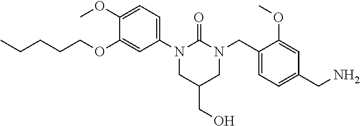 [SMILES: CCCCCOc1cc(N2CC(CO)CN(Cc3ccc(CN)cc3OC)C2=O)ccc1OC] | 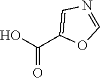 | 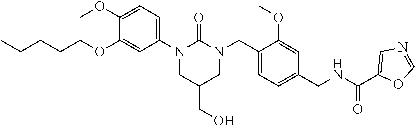 [SMILES: CCCCCOc1cc(N2CC(CO)CN(Cc3ccc(CNC(=O)c4cnco4)cc3OC)C2=O)ccc1OC] |
| | | Racemate |
| 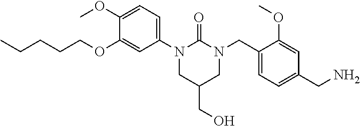 [SMILES: CCCCCOc1cc(N2CC(CO)CN(Cc3ccc(CN)cc3OC)C2=O)ccc1OC] | 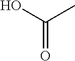 | 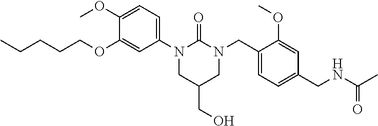 [SMILES: CCCCCOc1cc(N2CC(CO)CN(Cc3ccc(CNC(C)=O)cc3OC)C2=O)ccc1OC] |
| | | Racemate |
| 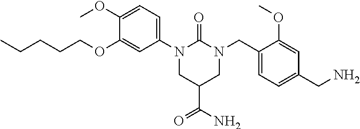 [SMILES: CCCCCOc1cc(N2CC(C(N)=O)CN(Cc3ccc(CN)cc3OC)C2=O)ccc1OC] | 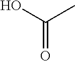 | 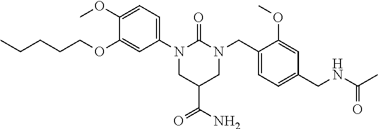 [SMILES: CCCCCOc1cc(N2CC(C(N)=O)CN(Cc3ccc(CNC(C)=O)cc3OC)C2=O)ccc1OC] |
| | | Racemate |

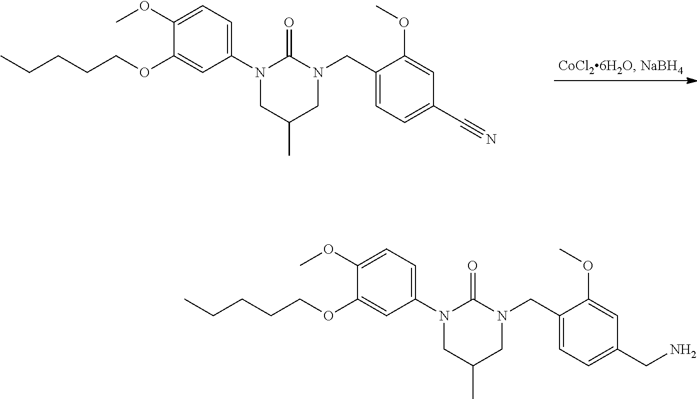 [SMILES: CCCCCOc1cc(N2CC(C)CN(Cc3ccc(C#N)cc3OC)C2=O)ccc1OC ; CCCCCOc1cc(N2CC(C)CN(Cc3ccc(CN)cc3OC)C2=O)ccc1OC]

To a solution of 3-methoxy-4-((3-(4-methoxy-3-(pentyloxy)phenyl)-5-methyl-2-oxotetrahydropyrimidin-1(2H)-yl)methyl)benzonitrile (903 mg, 2 mmol) in methanol (10 mL) is added cobalt chloride hexahydrate (2.37 g, 10 mmol) at −30° C. The reaction solution is stirred for 0.5 h, then sodium borohydride (757 mg, 20 mmol) is added portionwise at between −30° C. and −20° C. After stirred at this temperature for 1 h, the reaction mixture is allowed to warm to room temperature and stirred for another 2 h. The reaction mixture is cooled to 0° C., quenched by addition of water and then extracted with ethyl acetate. The combined organic layers are washed with brine, dried over anhydrous sodium sulfate, filtered and concentrated in vacuo. Crude 1-(4-(aminomethyl)-2-methoxybenzyl)-3-(4-methoxy-3-(pentyloxy)phenyl)-5-methyltetrahydropyrimidin-2(1H)-one is used for next step without further purification.

The following compounds are prepared in a similar manner as 1-(4-(aminomethyl)-2-methoxybenzyl)-3-(4-methoxy-3-(pentyloxy)phenyl)-5-methyltetrahydropyrimidin-2(1H)-one, as described above.

| Nitrile | Compound |
|---|---|
| 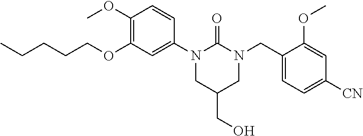 [SMILES: CCCCCOc1cc(N2CC(CO)CN(Cc3ccc(C#N)cc3OC)C2=O)ccc1OC] | 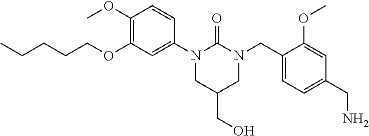 [SMILES: CCCCCOc1cc(N2CC(CO)CN(Cc3ccc(CN)cc3OC)C2=O)ccc1OC] |
| 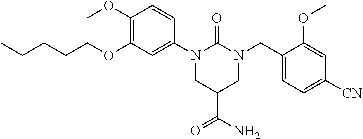 [SMILES: CCCCCOc1cc(N2CC(C(N)=O)CN(Cc3ccc(C#N)cc3OC)C2=O)ccc1OC] | 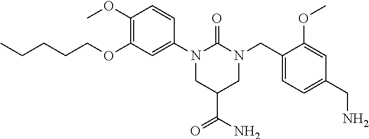 [SMILES: CCCCCOc1cc(N2CC(C(N)=O)CN(Cc3ccc(CN)cc3OC)C2=O)ccc1OC] |

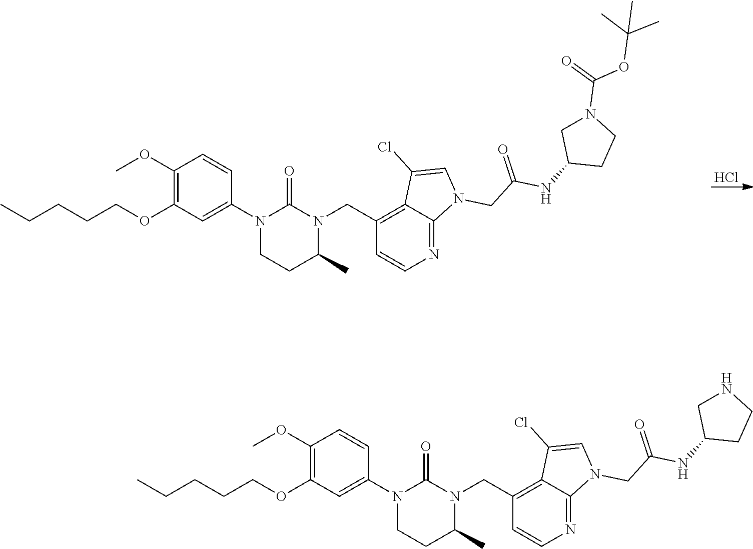

tert-Butyl (S)-3-(2-(3-chloro-4-(((S)-3-(4-methoxy-3-(pentyloxy)phenyl)-6-methyl-2-oxotetrahydropyrimidin-1(2H)-yl)methyl)-1H-pyrrolo[2,3-b]pyridin-1-yl)acetamido)pyrrolidine-1-carboxylate (41 mg, 59 μmol) is dissolved in dichloromethane (1 mL) and the solution is cooled to 0° C. before addition of 4 M hydrochloric acid solution in 1,4-dioxane (60 μL, 0.24 mmol). The reaction mixture is stirred at room temperature for 3 h before concentrated in vacuo. The residue is suspended in dichloromethane (10 mL) and cooled to 0° C. 2 N NaOH aqueous solution is added until pH>10. Aqueous layer is separated from organic layer and then extracted with dichloromethane two times. Combined organic layers are dried over anhydrous sodium sulfate, filtered and concentrated in vacuo to afford 2-(3-Chloro-4-(((S)-3-(4-methoxy-3-(pentyloxy)phenyl)-6-methyl-2-oxotetrahydropyimidin-1(2H)-yl)methyl)-1H-pyrrolo[2,3-b]pyridin-1-yl)-N—((S)-pyrrolidin-3-yl)acetamide (35 mg, 100% yield) as a white solid.

The following compounds are prepared in a similar manner as 2-(3-Chloro-4-(((S)-3-(4-methoxy-3-(pentyloxy)phenyl)-6-methyl-2-oxotetrahydropyrimidin-1(2H)-yl)methyl)-1H-pyrrolo[2,3-b]pyridin-1-yl)-N—((S)-pyrolidin-3-yl)acetamide, as described above.

| Substrate | Compound |
|---|---|
| 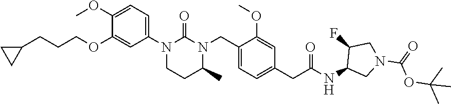 [SMILES: COc1cc(CC(=O)N[C@@H]2CN(C(=O)OC(C)(C)C)C[C@@H]2F)ccc1CN1C(=O)N(c2ccc(OC)c(OCCCC3CC3)c2)CC[C@@H]1C] | 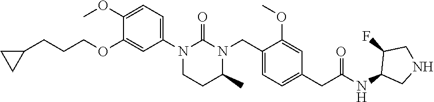 [SMILES: COc1cc(CC(=O)N[C@@H]2CNC[C@@H]2F)ccc1CN1C(=O)N(c2ccc(OC)c(OCCCC3CC3)c2)CC[C@@H]1C] |
| 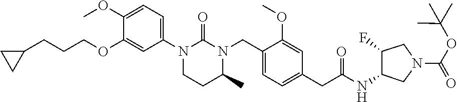 [SMILES: COc1cc(CC(=O)N[C@H]2CN(C(=O)OC(C)(C)C)C[C@H]2F)ccc1CN1C(=O)N(c2ccc(OC)c(OCCCC3CC3)c2)CC[C@@H]1C] | 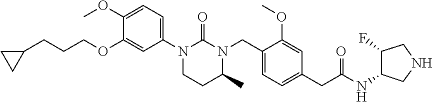 [SMILES: COc1cc(CC(=O)N[C@H]2CNC[C@H]2F)ccc1CN1C(=O)N(c2ccc(OC)c(OCCCC3CC3)c2)CC[C@@H]1C] |
| 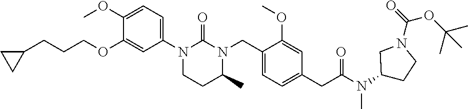 [SMILES: COc1cc(CC(=O)N(C)[C@H]2CCN(C(=O)OC(C)(C)C)C2)ccc1CN1C(=O)N(c2ccc(OC)c(OCCCC3CC3)c2)CC[C@@H]1C] | 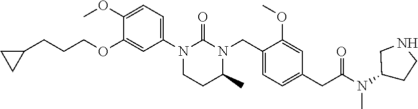 [SMILES: COc1cc(CC(=O)N(C)[C@H]2CCNC2)ccc1CN1C(=O)N(c2ccc(OC)c(OCCCC3CC3)c2)CC[C@@H]1C] |
| 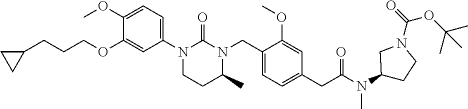 [SMILES: COc1cc(CC(=O)N(C)[C@@H]2CCN(C(=O)OC(C)(C)C)C2)ccc1CN1C(=O)N(c2ccc(OC)c(OCCCC3CC3)c2)CC[C@@H]1C] | 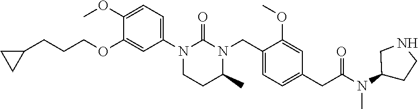 [SMILES: COc1cc(CC(=O)N(C)[C@@H]2CCNC2)ccc1CN1C(=O)N(c2ccc(OC)c(OCCCC3CC3)c2)CC[C@@H]1C] |
| 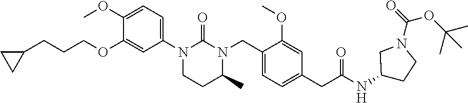 [SMILES: COc1cc(CC(=O)N[C@H]2CCN(C(=O)OC(C)(C)C)C2)ccc1CN1C(=O)N(c2ccc(OC)c(OCCCC3CC3)c2)CC[C@@H]1C] | 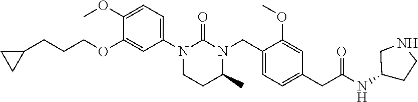 [SMILES: COc1cc(CC(=O)N[C@H]2CCNC2)ccc1CN1C(=O)N(c2ccc(OC)c(OCCCC3CC3)c2)CC[C@@H]1C] |
| 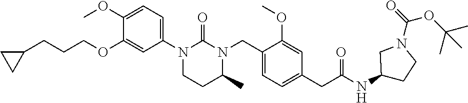 [SMILES: COc1cc(CC(=O)N[C@@H]2CCN(C(=O)OC(C)(C)C)C2)ccc1CN1C(=O)N(c2ccc(OC)c(OCCCC3CC3)c2)CC[C@@H]1C] | 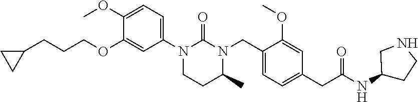 [SMILES: COc1cc(CC(=O)N[C@@H]2CCNC2)ccc1CN1C(=O)N(c2ccc(OC)c(OCCCC3CC3)c2)CC[C@@H]1C] |

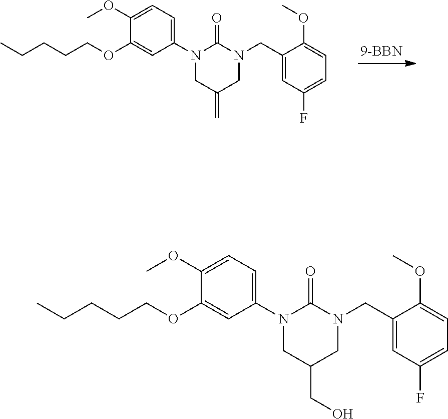 [SMILES: C=C1CN(Cc2cc(F)ccc2OC)C(=O)N(c2ccc(OC)c(OCCCCC)c2)C1 ; CCCCCOc1cc(N2CC(CO)CN(Cc3cc(F)ccc3OC)C2=O)ccc1OC]

To a solution of 1-(5-fluoro-2-methoxybenzyl)-3-(4-methoxy-3-(pentyloxy)phenyl)-5-methylenetetrahydropyrimidin-2(1H)-one (0.27 g, 0.60 mmol) in tetrahydrofuran (3 mL) at 0° C. is slowly added 9-borabicyclo[3.3.1]nonane (0.5 M in tetrahydrofuran, 1.44 ml, 0.72 mmol). The reaction solution is stirred at room temperature for 3 h before addition of a suspension of sodium perborate (276 mg) in water (3 ml). The mixture is stirred at room temperature for 16 h before filtration. The solid is washed with diethyl ether and the filtrate is extracted with diethyl ether two times. The combined diethyl ether layers are dried over anhydrous sodium sulfate, filtered and concentrated in vacuo. The residue is purified over silica gel by flash chromatography to give 1-(5-fluoro-2-methoxybenzyl)-5-(hydroxymethyl)-3-(4-methoxy-3-(pentyloxy)phenyl)tetrahydropyrimidin-2(1H)-one (0.20 g, 72% yield) as a white solid.

The following compounds are prepared in a similar manner as 1-(5-fluoro-2-methoxybenzyl)-5-(hydroxymethyl)-3-(4-methoxy-3-(pentyloxy)phenyl)tetrahydropyrimidin-2(1H)-one, as described above.

| Substrate | Compound |
|---|---|
| 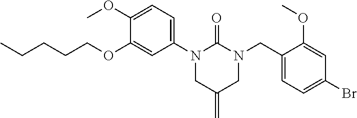 [SMILES: C=C1CN(Cc2ccc(Br)cc2OC)C(=O)N(c2ccc(OC)c(OCCCCC)c2)C1] | 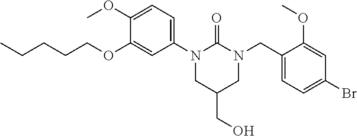 [SMILES: CCCCCOc1cc(N2CC(CO)CN(Cc3ccc(Br)cc3OC)C2=O)ccc1OC] |
| 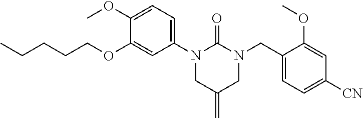 [SMILES: C=C1CN(Cc2ccc(C#N)cc2OC)C(=O)N(c2ccc(OC)c(OCCCCC)c2)C1] | 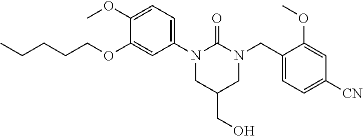 [SMILES: CCCCCOc1cc(N2CC(CO)CN(Cc3ccc(C#N)cc3OC)C2=O)ccc1OC] |

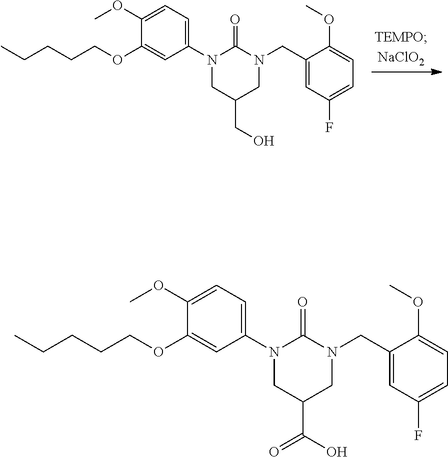 [SMILES: CCCCCOc1cc(N2CC(CO)CN(Cc3cc(F)ccc3OC)C2=O)ccc1OC ; CCCCCOc1cc(N2CC(C(=O)O)CN(Cc3cc(F)ccc3OC)C2=O)ccc1OC]

1-(5-Fluoro-2-methoxybenzyl)-5-(hydroxymethyl)-3-(4-methoxy-3-(pentyloxy)phenyl)tetrahydropyrimidin-2(1H)-one; 100 mg, 217 μmol) is dissolved in tetrahydrofuran (2.18 mL) and water (1.99 mL). It is then cooled to 0° C. Sodium phosphate monobasic monohydrate (449 mg, 3.26 mmol), iodobenzene diacetate (357 mg, 1.09 mmol) and 2,2,6,6-tetramethyl-1-piperidinyloxy (TEMPO; 17.3 mg, 109 μmol) are added sequentially to the reaction mixture. It is then warmed to room temperature and stirred for 1 h before cooled to 0° C. t-Butanol (1.00 mL) and 2-methyl-2-butene (1.17 mL, 10.9 mmol) are added followed by sodium chlorite (196 mg, 2.17 mmol). The mixture is warmed to room temperature and stirred for another 1 h. The reaction mixture is diluted with water and the aqueous layer is extracted with ethyl acetate three times. The combined organic layers are dried over anhydrous sodium sulfate, filtered and concentrated in vacuo. The residue is purified over silica gel by flash chromatography (20% ethyl acetate in heptanes to 100% ethyl acetate) to give 1-(5-fluoro-2-methoxybenzyl)-3-(4-methoxy-3-(pentyloxy)phenyl)-2-oxohexahydropyrimidine-5-carboxylic acid; 24.0 mg, 23% yield as an off-white solid.

The following compounds are prepared in a similar manner as 1-(5-fluoro-2-methoxybenzyl)-3-(4-methoxy-3-(pentyloxy)phenyl)-2-oxohexahydropyrimidine-5-carboxylic acid, as described above.

| Substrate | Compound |
|---|---|
| 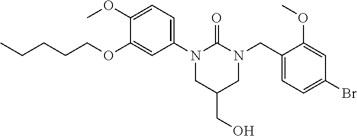 [SMILES: CCCCCOc1cc(N2CC(CO)CN(Cc3ccc(Br)cc3OC)C2=O)ccc1OC] | 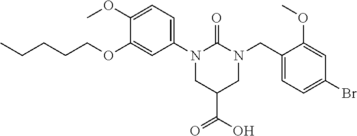 [SMILES: CCCCCOc1cc(N2CC(C(=O)O)CN(Cc3ccc(Br)cc3OC)C2=O)ccc1OC] |
| 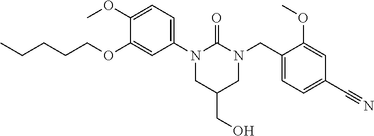 [SMILES: CCCCCOc1cc(N2CC(CO)CN(Cc3ccc(C#N)cc3OC)C2=O)ccc1OC] | 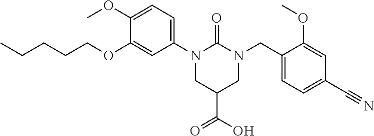 [SMILES: CCCCCOc1cc(N2CC(C(=O)O)CN(Cc3ccc(C#N)cc3OC)C2=O)ccc1OC] |

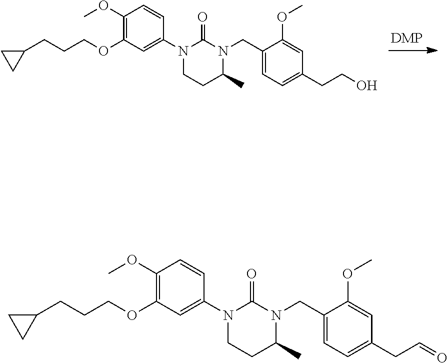 [SMILES: COc1cc(CCO)ccc1CN1C(=O)N(c2ccc(OC)c(OCCCC3CC3)c2)CC[C@@H]1C ; COc1cc(CC=O)ccc1CN1C(=O)N(c2ccc(OC)c(OCCCC3CC3)c2)CC[C@@H]1C]

To a stirred solution of (S)-1-(3-(3-cyclopropylpropoxy)-4-methoxyphenyl)-3-(4-(2-hydroxyethyl)-2-methoxybenzyl)-4-methyltetrahydropyrimidin-2(1H)-one (150 mg, 311 μmol) in dichloromethane (1.11 mL) at 0° C. is added 1,1,1-tris(acetyloxy)-1,1-dihydro-1,2-benziodoxol-3-(1H)-one (DMP; 139 mg, 311 μmol) in one portion. The mixture is stirred for 2 h at room temperature before quenched with a v/v 1:1 mixture of saturated sodium thiosulfate aqueous solution (1 mL) and saturated sodium bicarbonate aqueous solution (1 mL). The resulting mixture is extracted with dichloromethane two times. The combined organic layers are washed with brine, dried over anhydrous sodium sulfate, filtered and concentrated in vacuo. Crude (S)-2-(4-((3-(3-(3-cyclopropylpropoxy)-4-methoxyphenyl)-6-methyl-2-oxotetrahydropyrimidin-1(2H)-yl)methyl)-3-methoxyphenyl)acetaldehyde) is obtained as an oil which was used without further purification.

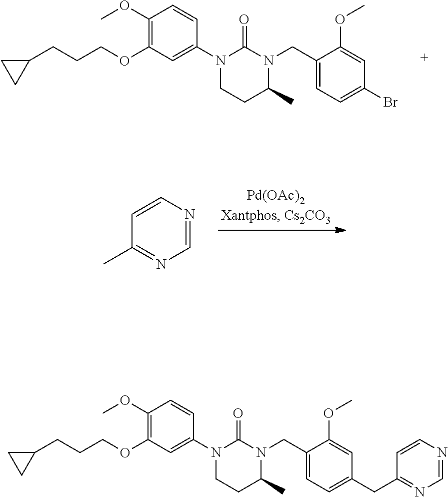

To a small vial are added (S)-3-(4-bromo-2-methoxybenzyl)-1-(3-(3-cyclopropylpropoxy)-4-methoxyphenyl)-4-methyltetrahydropyrimidin-2(1H)-one (78 mg, 0.15 mmol), 4-methylpyrimidine (14.6 mg, 0.15 mmol), cesium carbonate (99.2 mg, 0.30 mmol), 4,5-bis(diphenylphosphino)-9,9-dimethylxanthene (2.22 mg, 3.7 μmol) and palladium(II) acetate (0.85 mg, 3.7 μmol). The vial is purged with nitrogen then 1,4-dioxane (1.21 mL) was added, the vial is purged again with nitrogen and the reaction mixture is heated to 100° C. and stirred at 100° C. for 16 hours before cooled to room temperature. The mixture is diluted with ethyl acetate and filtered through a celite pad. The filtrate is concentrated under reduced pressure to afford a yellow oil. The crude oil is purified by reverse phase chromatography. (S)-1-(3-(3-Cyclopropylpropoxy)-4-methoxyphenyl)-3-(2-methoxy-4-(pyrimidin-4-ylmethyl)benzyl)-4-methyltetrahydropyrimidin-2(1H)-one (14.1 mg, 17% yield) was obtained as a yellow solid.

The following compounds are prepared in a similar manner as (S)-1-(3-(3-Cyclopropylpropoxy)-4-methoxyphenyl)-3-(2-methoxy-4-(pyrimidin-4-ylmethyl)benzyl)-4-methyltetrahydropyrimidin-2(1H)-one, as described above.

| Substrate | Reactant | Compound |
|---|---|---|
| 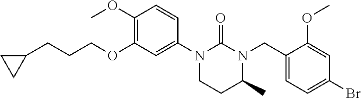 [SMILES: COc1cc(Br)ccc1CN1C(=O)N(c2ccc(OC)c(OCCCC3CC3)c2)CC[C@@H]1C] | 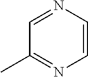 | 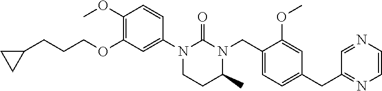 [SMILES: COc1cc(Cc2cnccn2)ccc1CN1C(=O)N(c2ccc(OC)c(OCCCC3CC3)c2)CC[C@@H]1C] |
| 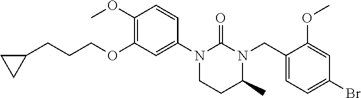 [SMILES: COc1cc(Br)ccc1CN1C(=O)N(c2ccc(OC)c(OCCCC3CC3)c2)CC[C@@H]1C] | 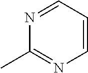 | 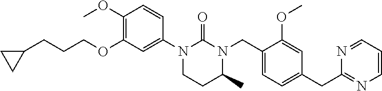 [SMILES: COc1cc(Cc2ncccn2)ccc1CN1C(=O)N(c2ccc(OC)c(OCCCC3CC3)c2)CC[C@@H]1C] |

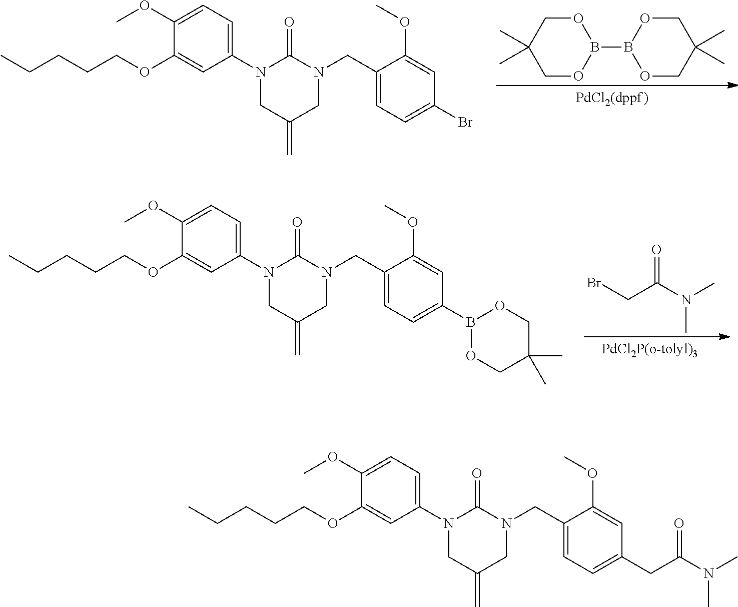

In a small vial fitted with a stir bar is combined 1,1′-bis(diphenylphosphino)ferrocene dichlorodpalladium(II) (45.0 mg, 61.5 μmol), potassium acetate (456 mg, 4.60 mmol), 1-(4-bromo-2-methoxybenzyl)-3-(4-methoxy-3-(pentyloxy)phenyl)-5-methylenetetrahydropyrimidin-2(1H)-one (0.77 g, 1.53 mmol), and bis(neopentyl glycolato)diboron (401 mg, 1.69 mmol). The vial is sealed with a septum and purged with nitrogen. Dry 1,4-dioxane (4.64 mL) is added via syringe and the suspension is further bubbled with nitrogen before sealing and heating to 80° C. for 16 h. The mixture is filtered through a celite pad and concentrated to dryness to provide crude 1-(4-(5,5-dimethyl-1,3,2-dioxaborinan-2-yl)-2-methoxybenzyl)-3-(4-methoxy-3-(pentyloxy)phenyl)-5-methylenetetrahydropyrimidin-2(1H)-one as a black oil, which is used for next step without purification.

1-(4-Methoxy-3-(pentyloxy)phenyl)-3-(2-methoxy-4-(4,4,5,5-tetramethyl-1,3,2-dioxaborolan-2-yl)benzyl)-5-methylenetetrahydropyrimidin-2(1H)-one (596 mg, 1.08 mmol), dichlorobis(tri-o-tolylphosphine)palladium(II) (20.2 mg, 24.9 μmol), cesium carbonate (428 mg, 1.30 mmol), 2-bromo-N,N-dimethylacetamide (246 μL, 2.17 mmol), 1,4-dioxane (2.02 mL) and water (804 μL) are added to a microwave vial. The vial is degassed by N₂ for 10 minutes and heated to 90° C. for 2 h. The reaction mixture is filtered over celite and washed with dichloromethane. The filtrate solution is concentrated in vacuo. The crude material is purified with reverse phase flash chromatography to afford 2-(3-methoxy-4-((3-(4-methoxy-3-(pentyloxy)phenyl)-5-methylene-2-oxotetrahydropyrimidin-1(2H)-yl)methyl)phenyl)-N,N-dimethylacetamide (82 mg, 15% yield) as a viscous colorless oil.

The following compounds are prepared in a similar manner as 2-(3-methoxy-4-((3-(4-methoxy-3-(pentyloxy)phenyl)-5-methylene-2-oxotetrahydropyrimidin-1(2H)-yl)methyl)phenyl)-N,N-dimethylacetamide, as described above.

| Substrate | Bromide | Compound |
|---|---|---|
| 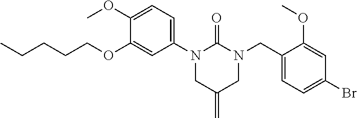 [SMILES: C=C1CN(Cc2ccc(Br)cc2OC)C(=O)N(c2ccc(OC)c(OCCCCC)c2)C1] | 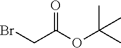 | 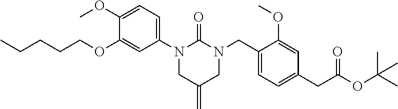 [SMILES: C=C1CN(Cc2ccc(CC(=O)OC(C)(C)C)cc2OC)C(=O)N(c2ccc(OC)c(OCCCCC)c2)C1] |
| 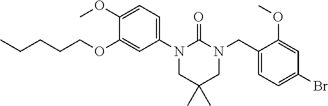 [SMILES: CCCCCOc1cc(N2CC(C)(C)CN(Cc3ccc(Br)cc3OC)C2=O)ccc1OC] | 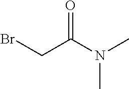 | 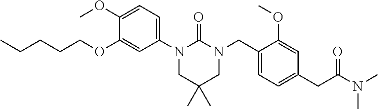 [SMILES: CCCCCOc1cc(N2CC(C)(C)CN(Cc3ccc(CC(=O)N(C)C)cc3OC)C2=O)ccc1OC] |
| 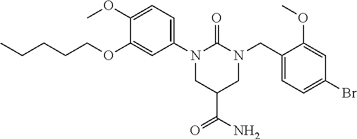 [SMILES: CCCCCOc1cc(N2CC(C(N)=O)CN(Cc3ccc(Br)cc3OC)C2=O)ccc1OC] | 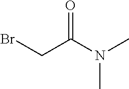 | 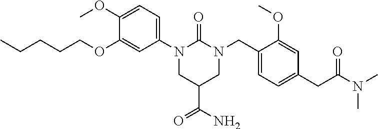 [SMILES: CCCCCOc1cc(N2CC(C(N)=O)CN(Cc3ccc(CC(=O)N(C)C)cc3OC)C2=O)ccc1OC] |
| 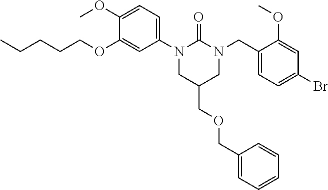 [SMILES: CCCCCOc1cc(N2CC(COCc3ccccc3)CN(Cc3ccc(Br)cc3OC)C2=O)ccc1OC] | 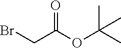 | 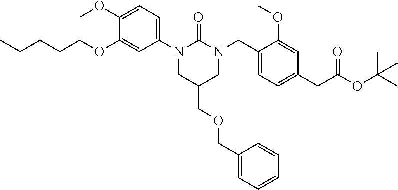 [SMILES: CCCCCOc1cc(N2CC(COCc3ccccc3)CN(Cc3ccc(CC(=O)OC(C)(C)C)cc3OC)C2=O)ccc1OC] |
| 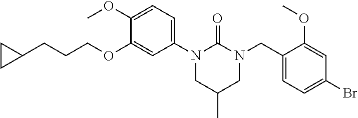 [SMILES: COc1cc(Br)ccc1CN1CC(C)CN(c2ccc(OC)c(OCCCC3CC3)c2)C1=O] | 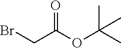 | 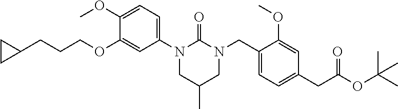 [SMILES: COc1cc(CC(=O)OC(C)(C)C)ccc1CN1CC(C)CN(c2ccc(OC)c(OCCCC3CC3)c2)C1=O] |

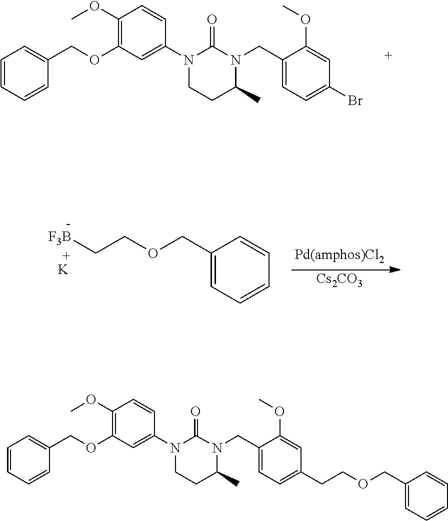

In a high pressure sealed flask are introduced bis(di-tert-butyl(4-dimethylaminophenyl)phosphine)dichloropalladium(II) (Pd(amphos)Cl₂, 175 mg, 242 μmol), cesium carbonate (4.76 g, 14.5 mmol), (S)-1-(3-(benzyloxy)-4-methoxyphenyl)-3-(4-bromo-2-methoxybenzyl)-4-methyltetrahydropyrimidin-2(1H)-one; 2.54 g, 4.83 mmol) and potassium (2-(benzyloxy)ethyl)trifluoroborate; 1.29 g, 5.32 mmol). The vial is sealed with a cap and degassed with nitrogen balloon. Degassed toluene (14.1 mL) and water (3.52 mL) are added by syringe. The reaction mixture is degassed for another 5 min. The reaction mixture is stirred at 100° C. for 20 h. After cooling to rt, the reaction mixture is diluted with water and extracted with ethyl acetate three times. The combined organic layers are dried over anhydrous sodium sulfate, filtered and concentrated in vacuo. The residue is purified over silica gel by flash chromatography (20% ethyl acetate in hexanes) to give (S)-1-(3-(benzyloxy)-4-methoxyphenyl)-3-(4-(2-(benzyloxy)ethyl)-2-methoxybenzyl)-4-methyltetrahydropyrimidin-2(1H)-one (2.35 g, 69% yield) as a yellowish oil.

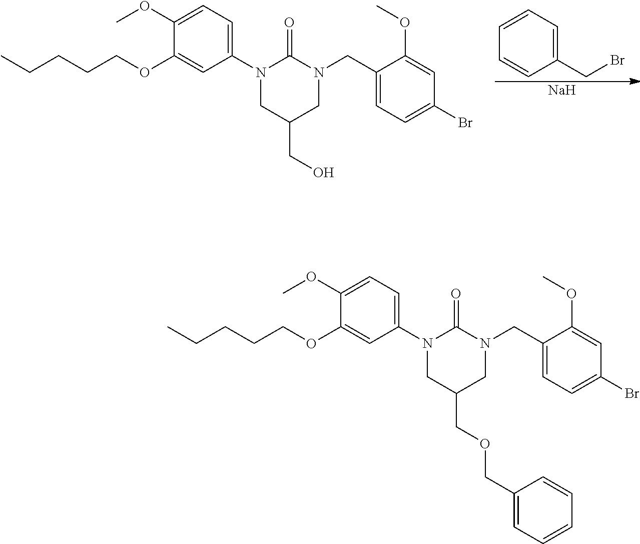

To a solution of 1-(4-bromo-2-methoxybenzyl)-5-(hydroxymethyl)-3-(4-methoxy-3-(pentyloxy)phenyl)tetrahydropyrimidin-2(1H)-one (3.10 g, 5.94 mmol) in tetrahydrofuran (29.7 mL) is added sodium hydride (60% dispersion in mineral oil, 476 mg, 11.9 mmol) at 0° C. and the resulting mixture is stirred for 5 min at this temperature. Then, benzyl bromide (865 μL, 7.13 mmol) and tetrabutylammonium iodide (896 mg, 2.38 mmol) are added to the reaction mixture. Reaction mixture is warmed to room temperature and stirred for 1 h. Another portion of sodium hydride (60% dispersion in mineral oil, 280 mg, 7 mmol) and benzyl bromide (100 μl, 0.82 mmol) are added and reaction mixture stirred for another 1 h before quenched with water. The mixture is extracted with ethyl acetate three times. Combined organic layers are dried over sodium sulfate, filtered and concentrated in vacuo. The residue is purified over silica gel by flash chromatography to give 5-((benzyloxy)methyl)-1-(4-bromo-2-methoxybenzyl)-3-(4-methoxy-3-(pentyloxy)phenyl)tetrahydropyrimidin-2(1H)-one (2.50 g, 69% yield) as a yellow oil.

The following compounds are prepared in a similar manner as 5-((benzyloxy)methyl)-1-(4-bromo-2-methoxybenzyl)-3-(4-methoxy-3-(pentyloxy)phenyl)tetrahydropyrimidin-2(1H)-one, as described above.

| Substrate | Alkylation reagent | Compound |
|---|---|---|
| 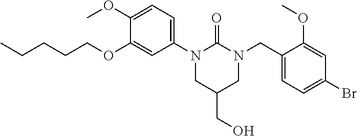 [SMILES: CCCCCOc1cc(N2CC(CO)CN(Cc3ccc(Br)cc3OC)C2=O)ccc1OC] | 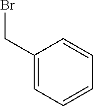 | 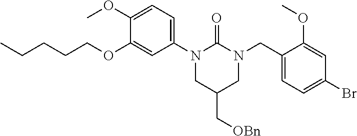 [SMILES: CCCCCOc1cc(N2CC(COCc3ccccc3)CN(Cc3ccc(Br)cc3OC)C2=O)ccc1OC] |

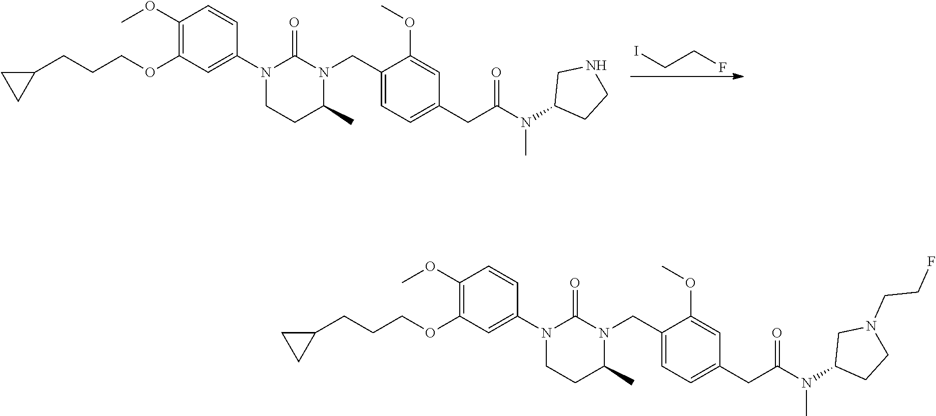

To a solution of 2-(4-(((S)-3-(3-(3-cyclopropylpropoxy)-4-methoxyphenyl)-6-methyl-2-oxotetrahydropyrimidin-1(2H)-yl)methyl)-3-methoxyphenyl)-N-methyl-N—((S)-pyrrolidin-3-yl)acetamide (75.0 mg, 130 μmol) in N,N-dimethylformamide (288 μL) at room temperature are added N,N-diisopropylethylamine (68.1 μL, 389 μmol) and 1-fluoro-2-iodoethane (29.9 mg, 168 μmol). The reaction mixture is stirred at room temperature for 16 h. It is directly purified with prep-HPLC to give 2-(4-(((S)-3-(3-(3-cyclopropylpropoxy)-4-methoxyphenyl)-6-methyl-2-oxotetrahydropyrimidin-1(2H)-yl)methyl)-3-methoxyphenyl)-N—((S)-1-(2-fluoroethyl)pyrrolidin-3-yl)-N-methylacetamide (13.0 mg, 16% yield) as a pale yellow solid.

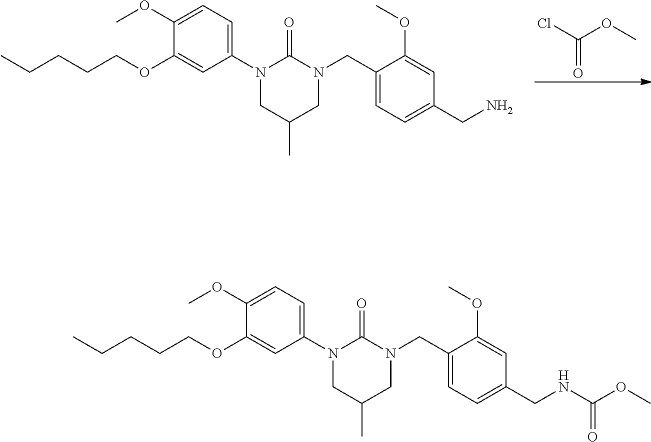

To 1-(4-(aminomethyl)-2-methoxybenzyl)-3-(4-methoxy-3-(pentyloxy)phenyl)-5-methyltetrahydropyrimidin-2(1H)-one; 45 mg, 0.10 mmol) in dichloromethane (1 mL) at 0° C. are added methyl chloroformate (8.5 μL, 0.11 mmol) and triethylamine (16.7 μL, 1.2 mmol). The reaction solution is stirred at room temperature for 2 h before quenched with saturated ammonium chloride aqueous solution. The mixture is extracted with dichloromethane two times and the combined organic layers are washed with brine, dried over anhydrous sodium sulfate, filtered and concentrated. The residue is purified over silica gel by flash chromatography to give methyl (3-methoxy-4-((3-(4-methoxy-3-(pentyloxy)phenyl)-5-methyl-2-oxotetrahydropyrimidin-1(2H)-yl)methyl)benzyl)carbamate).

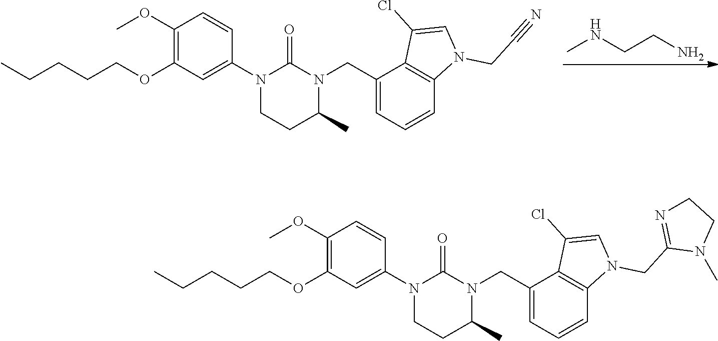

To a stirring solution of N-methylethane-1,2-diamine (74 mgs, 1 mmol) in toluene (5 mL) is added dropwise trimethyl aluminum (2.0 M in toluene, 1 mL) at room temperature. The resulting mixture is stirred at room temperature for 1 hour whereupon (S)-2-(3-chloro-4-((3-(4-methoxy-3-(pentyloxy)phenyl)-6-methyl-2-oxotetrahydropyrimidin-1-(2H)-yl) methyl)-1H-indol-1-yl) acetonitrile (100 mg, 0.2 mmol) is added neat. The resulting slurry is heated for 1 h at 90° C. then cooled to room temperature and quenched with saturated ammonium chloride aqueous solution. The mixture is extracted with dichloromethane three times and the combined organic layers are washed with brine, dried over anhydrous sodium sulfate, filtered and concentrated in vacuo. The crude is purified using a C18 reverse phase column eluting with 0-100% acetonitrile in water (containing 0.15% trifluoroacetic acid) over 15 min. (S)-3-((Chloro-1-((1-methyl-4,5-dihydro-1H-imidazol-2-yl)methyl)-1H-indol-4-yl) methyl)-1-(4-methoxy-3-(pentyl oxy) phenyl)-4-methyltetrahydropyrimidin-2(1H)-one mono trifluoroacetic acid salt (29 mg, 0.05 mmol, 19% yield) is obtained as a white solid.

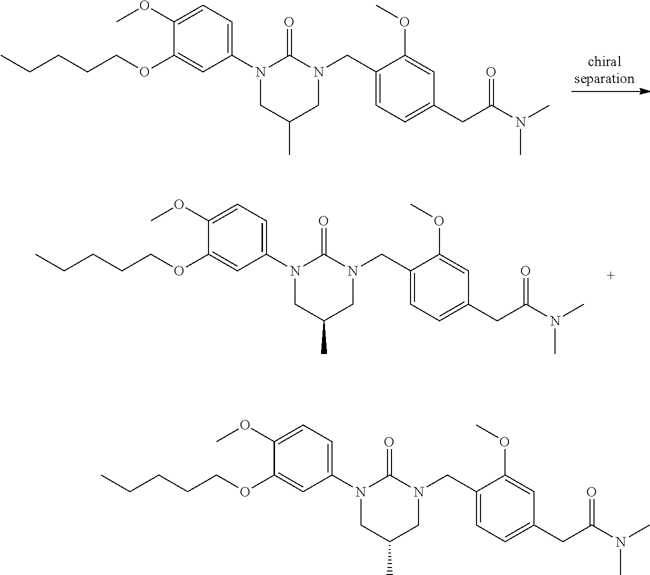

Racemic mixture of 2-(3-methoxy-4-((3-(4-methoxy-3-(pentyloxy)phenyl)-5-methyl-2-oxotetrahydropyrimidin-1(2H)-yl)methyl)phenyl)-N,N-dimethylacetamide is purified by preparative supercritical fluid chromatography to give two enantiomeric pure compounds. Purification method is described here. Column: Lux Amylose-2, 10×250 mm 5 μm, precolumn: Lux Amylose-2, 10×10 mm 5 μm, mobile phase: 60% acetonitrile:ethanol/40% supercritical carbon dioxide, mode: Isocratic, flow rate: 10 mL/min, backpressure: 150 bar, column temperature: 40° C., run time: 15 min. Enantiomer #1: 6.0 min, enantiomeric excess ≥99.9%; enantiomer #2: 11.5 min, enantiomeric excess ≥99.0%.

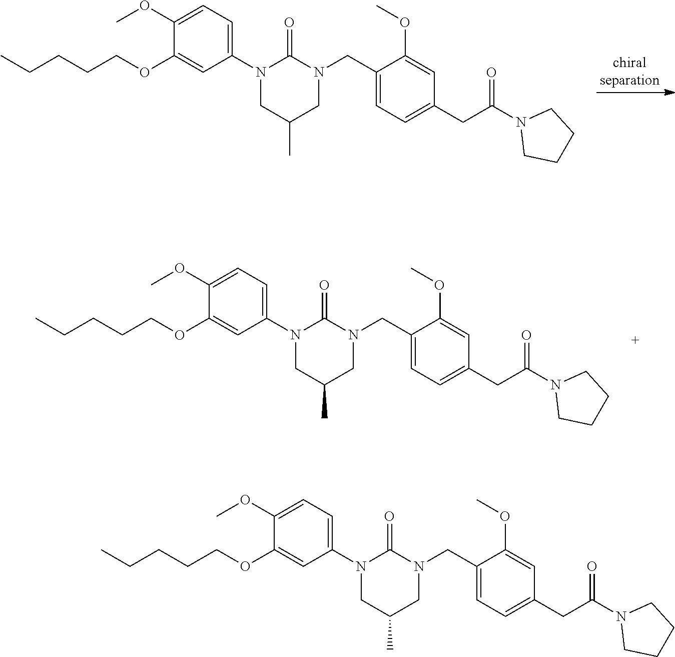 [SMILES: CCCCCOc1cc(N2CC(C)CN(Cc3ccc(CC(=O)N4CCCC4)cc3OC)C2=O)ccc1OC ; CCCCCOc1cc(N2C[C@H](C)CN(Cc3ccc(CC(=O)N4CCCC4)cc3OC)C2=O)ccc1OC ; CCCCCOc1cc(N2C[C@@H](C)CN(Cc3ccc(CC(=O)N4CCCC4)cc3OC)C2=O)ccc1OC]

Racemic mixture of 1-(4-methoxy-3-(pentyloxy)phenyl)-3-(2-methoxy-4-(2-oxo-2-(pyrrolidin-1-yl)ethyl)benzyl)-5-methyltetrahydropyrimidin-2(1H)-one is purified by preparative supercritical fluid chromatography to give two enantiomeric pure compounds. Purification method is described here. Column: Lux Amylose-2, 10×250 mm 5 μm, precolumn: Lux Amylose-2, 10×10 mm 5 μm, mobile phase: 60% acetonitrile:ethanol/40% supercritical carbon dioxide, mode: Isocratic, flow rate: 10 mL/min, backpressure: 150 bar, column temperature: 40° C., run time: 26 min. Enantiomer #1: 9.3 min, enantiomeric excess ≥98.7%; enantiomer #2: 21.3 min, enantiomeric excess ≥99.4%.

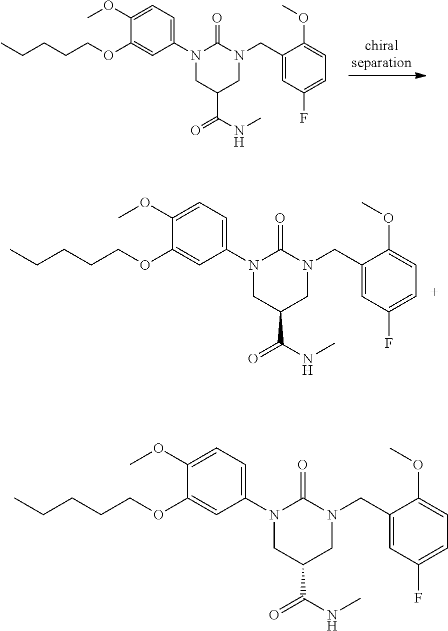 [SMILES: CCCCCOc1cc(N2CC(C(=O)NC)CN(Cc3cc(F)ccc3OC)C2=O)ccc1OC ; CCCCCOc1cc(N2C[C@H](C(=O)NC)CN(Cc3cc(F)ccc3OC)C2=O)ccc1OC ; CCCCCOc1cc(N2C[C@@H](C(=O)NC)CN(Cc3cc(F)ccc3OC)C2=O)ccc1OC]

Racemic mixture of 1-(5-fluoro-2-methoxybenzyl)-3-(4-methoxy-3-(pentyloxy)phenyl)-N-methyl-2-oxohexahydropyrimidine-5-carboxamide is purified by preparative supercritical fluid chromatography to give two enantiomeric pure compounds. Purification method is described here. Column: IC, ChiralPak, 4×6×250 mm 5 μm, mobile phase: 30% methanol/70% supercritical carbon dioxide, mode: Isocratic, flow rate: 4 mL/min, backpressure: 150 bar, column temp: 40° C., run time: 25 min. Enantiomer #1: 12.1 min, enantiomeric excess ≥99.8%; enantiomer #2: 14.5 min, enantiomeric excess ≥99.9%.

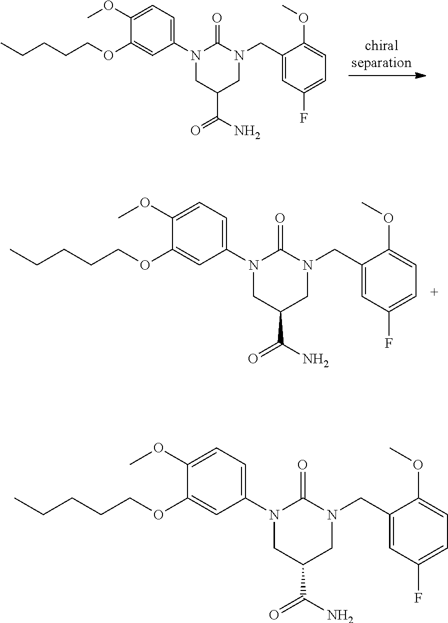 [SMILES: CCCCCOc1cc(N2CC(C(N)=O)CN(Cc3cc(F)ccc3OC)C2=O)ccc1OC ; CCCCCOc1cc(N2C[C@H](C(N)=O)CN(Cc3cc(F)ccc3OC)C2=O)ccc1OC ; CCCCCOc1cc(N2C[C@@H](C(N)=O)CN(Cc3cc(F)ccc3OC)C2=O)ccc1OC]

Racemic mixture of 1-(5-fluoro-2-methoxybenzyl)-3-(4-methoxy-3-(pentyloxy)phenyl)-2-oxohexahydropyrimidine-5-carboxamide is purified by preparative supercritical fluid chromatography to give two enantiomeric pure compounds. Purification method is described here. Column: Lux Amylose-2, 10×250 mm 5 μm, mobile phase: 60% isopropanol/40% supercritical carbon dioxide, mode: Isocratic, flow rate: 10 mL/min, backpressure: 150 bar, column temperature: 40° C., run time: 7 min. Enantiomer #1: 3.1 min, enantiomeric excess ≥96.9%; enantiomer #2: 5.3 min, enantiomeric excess ≥98.1%.

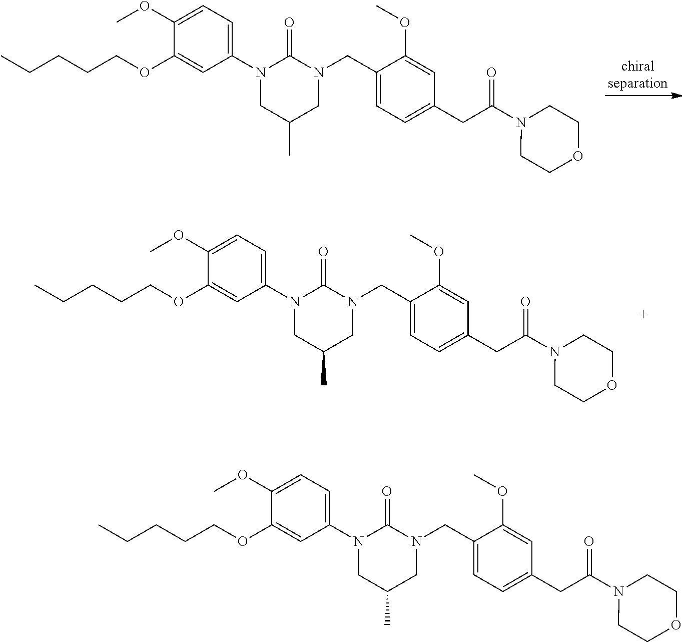 [SMILES: CCCCCOc1cc(N2CC(C)CN(Cc3ccc(CC(=O)N4CCOCC4)cc3OC)C2=O)ccc1OC ; CCCCCOc1cc(N2C[C@H](C)CN(Cc3ccc(CC(=O)N4CCOCC4)cc3OC)C2=O)ccc1OC ; CCCCCOc1cc(N2C[C@@H](C)CN(Cc3ccc(CC(=O)N4CCOCC4)cc3OC)C2=O)ccc1OC]

Racemic mixture of 1-(4-methoxy-3-(pentyloxy)phenyl)-3-(2-methoxy-4-(2-morpholino-2-oxoethyl)benzyl)-5-methyltetrahydropyrimidin-2(1H)-one is purified by preparative HPLC to give two enantiomeric pure compounds. Purification method is described here. Column: ChiralPak IA, 250 mm×4.6 mm ID, 5 μm, mobile phase: IA, v/v/v 5:30:65 ethanol:dichloromethane:hexane, mode: Isocratic, flow rate: 0.8 mL/min, backpressure: 57 bar, column temp: 26° C., run time: 26 min. Enantiomer #1: 23.5 min, enantiomeric excess ≥98.9%; enantiomer #2: 25.5 min, enantiomeric excess ≥96.4%.

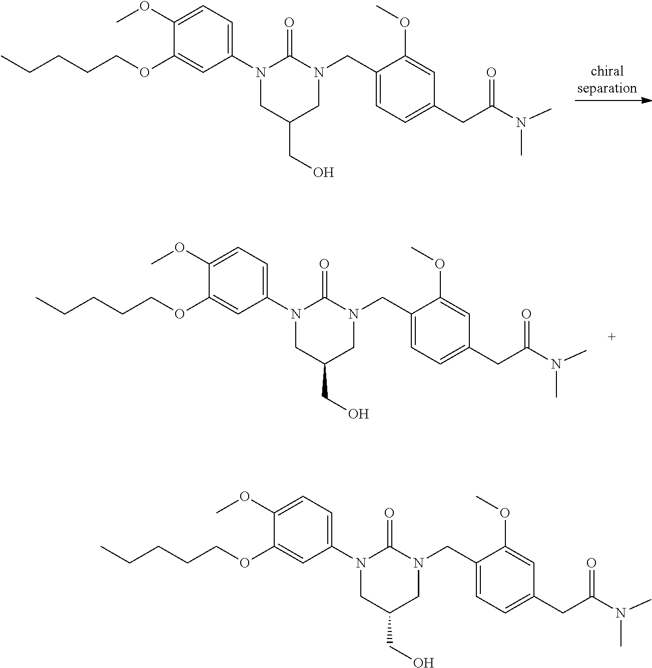

Racemic mixture of 2-(4-((5-(hydroxymethyl)-3-(4-methoxy-3-(pentyloxy)phenyl)-2-oxotetrahydropyrimidin-1(2H)-yl)methyl)-3-methoxyphenyl)-N,N-dimethylacetamide is purified by preparative supercritical fluid chromatography to give two enantiomeric pure compounds. Purification method is described here. Column: Lux Amylose-2, 10×250 mm 5 um, precolumn: Lux iAmylose-2, 10×10 mm 5 μm, mobile phase: 40% acetonitrile:ethanol/60% supercritical carbon dioxide, mode: Isocratic, flow rate: 10 mL/min, backpressure: 150 bar, column temp: 40° C., run time: 15 min. Enantiomer #1: 8.1 min, enantiomeric excess ≥99.3%, enantiomer #2: 13.4 min, enantiomeric excess ≥99.5%.

1. A compound having a structure of Formula (700): 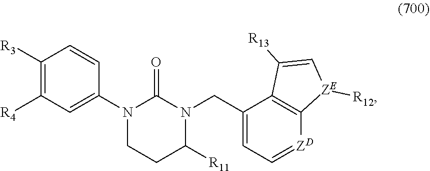
or a pharmaceutically acceptable salt thereof, wherein:
R3 and R4 are independently an alkyl, cyclic alkyl, alkenyl, alkynyl, halogen, hydroxyl, alkoxyl, ether, CN, amine, amide, aryl, or heteroaryl, wherein the alkyl, cyclic alkyl, alkenyl, alkynyl, alkoxyl, ether, amine, aryl, or heteroaryl is optionally substituted;
R11 is H or an alkyl group, wherein the alkyl group is optionally substituted;
R12 is H, an alkyl, alkenyl, alkynyl, halogen, hydroxyl, alkoxyl, ether, CN, amine, amide, aryl, heteroaryl, cyclic alkyl, heterocyclic alkyl, multicyclic alkyl group, or hetero multicyclic alkyl group, wherein the alkyl, alkenyl, alkynyl, alkoxy, ether, amine, aryl, heteroaryl group, cyclic alkyl, heterocyclic alkyl, multicyclic alkyl, or hetero multicyclic alkyl group is optionally substituted;
R13 is H, a halogen, —CN, —CF3, or a C1-C3 alkyl group;
Z^{D} is N or CR₁₄, wherein R14 is H or an alkyl group, wherein the alkyl group is optionally substituted; and
Z^{E} is N or CH.

2. The compound of claim 1, wherein R3 is —OCH3.

3. The compound of claim 1, wherein R4 is an alkoxyl group.

4. The comp nd of claim 3, wherein R4 is

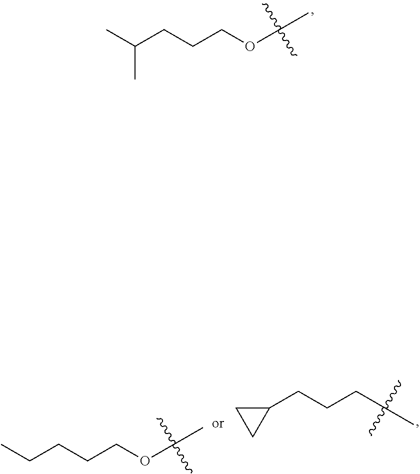

5. The compound of claim 1, wherein R12 comprises an amide group.

6. A compound having a structure of Formula (701): 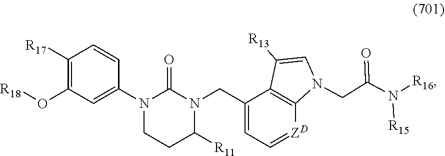
or a pharmaceutically acceptable salt thereof, wherein:
R11 is H or a methyl group;
R13 is H, halogen, —CN, —CF3, or a C1-C3 alkyl group;
R15 and R16 are independently a H, alkyl, aryl, heteroaryl, cyclic alkyl, heterocyclic alkyl, multicyclic alkyl group, or hetero multicyclic alkyl group, wherein the alkyl, aryl, heteroaryl group, cyclic alkyl, heterocyclic alkyl, multicyclic alkyl, or hetero multicyclic alkyl group is optionally substituted, wherein R15 and R16, together with the nitrogen they are attached to, optionally form a 3 to 8 membered heterocyclic group, wherein the heterocyclic group may be optionally substituted;
R17 is a halogen, an alkyl group, or an alkoxyl group;
R18 is an alkyl group; and
Z^{D} is N or CR₁₄, wherein R14 is H or an alkyl group, wherein the alkyl group is optionally substituted.

7-26. (canceled)

27. A compound having a structure selected from the group consisting of SM0001, SM0002, SM0003, SM0004, SM0005, SM0006, SM0007, SM0008, SM0009, SM0010, SM0011, SM0012, SM0013, SM0014, SM0015, SM0016, SM0017, SM0018, SM0019, SM0020, SM0021, SM0022, SM0023, SM0024, SM0025, SM0026, SM0027, SM0028, SM0029, SM0030, SM0031, SM0032, SM0033, SM0034, SM0035, SM0036, SM0037, SM0038, SM0039, SM0040, SM0041, SM0042, SM0043, SM0044, SM0045, SM0046, SM0047, SM0048, SM0049, SM0050, SM0051, SM0052, SM0053, SM0054, SM0055, SM0056, SM0057, SM0058, SM0059, SM0060, SM0061, SM0062, SM0063, SM0064, SM0065, SM0066, SM0067, SM0068, SM0069, SM0070, SM0071, SM0072, SM0073, SM0074, SM0075, SM0076, SM0077, SM0078, SM0079, SM0080, SM0081, SM0082, SM0083, SM0084, SM0085, SM0086, SM0087, SM0088, SM0089, SM0090, SM0091, SM0092, SM0093, SM0094, SM0095, SM0096, SM0097, SM0098, SM0099, SM0100, SM0101, SM0102, SM0103, SM0104, SM0105, SM0106, SM0107, SM0108, SM0109, SM0110, SM0111, SM0112, SM0113, SM0114, SM0115, SM0116, SM0117, SM0118, SM0119, SM0120, SM0121, SM0200, SM0201, SM0202, SM0203, SM0204, SM0205, SM0206, SM0207, SM0208, SM0209, SM0210, SM0211, SM0212, SM0213, SM0214, SM0215, SM0216, SM0217, SM0218, SM0219, C5INH-0294, C5INH-0296, C5INH-0298, C5INH-0303, C5INH-0310, C5INH-0311, C5INH-0315, C5INH-0316, C5INH-0317, C5INH-0318, C5INH-0319, C5INH-0321, C5INH-0323, C5INH-0324, C5INH-0326, C5INH-0329, C5INH-0330, C5INH-0333, C5INH-0335, C5INH-0336, C5INH-0338, C5INH-0339, C5INH-0340, C5INH-0342, C5INH-0343, C5INH-0348, C5INH-0349, C5INH-0350, C5INH-0352, C5INH-0353, C5INH-0355, C5INH-0356, C5INH-0357, C5INH-0361, C5INH-0366, C5INH-0367, C5INH-0369, C5INH-0370, C5INH-0371, C5INH-0372, C5INH-0373, C5INH-0377, C5INH-0379, C5INH-0381, C5INH-0382, C5INH-0383, C5INH-0384, C5INH-0385, C5INH-0387, C5INH-0388, C5INH-0389, C5INH-0390, C5INH-0391, C5INH-0395, C5INH-0396, C5INH-0397, C5INH-0398, C5INH-0399, C5INH-0401, C5INH-0402, C5INH-0403, C5INH-0406, C5INH-0409, C5INH-0410, C5INH-0411, C5INH-0414, C5INH-0417, C5INH-0420, C5INH-0421, C5INH-0422, C5INH-0425, C5INH-0428, C5INH-0431, C5INH-0432, C5INH-0436, C5INH-0437, C5INH-0438, C5INH-0440, C5INH-0443, C5INH-0446, C5INH-0447, C5INH-0448, C5INH-0450, C5INH-0452, C5INH-0453, C5INH-0454, C5INH-0456, C5INH-0458, C5INH-0460, C5INH-0462, C5INH-0463, C5INH-0469, C5INH-0472, C5INH-0473, C5INH-0474, C5INH-0476, C5INH-0477, C5INH-0484, C5INH-0485, C5INH-0486, C5INH-0487, C5INH-0488, C5INH-0489, C5INH-0490, C5INH-0491, C5INH-0492, C5INH-0496, C5INH-0497, C5INH-0498, C5INH-0500, C5INH-0501, C5INH-0502, C5INH-0504, C5INH-0507, C5INH-0508, C5INH-0509, C5INH-0510, C5INH-0512, C5INH-0513, C5INH-0515, C5INH-0516, C5INH-0517, C5INH-0518, C5INH-0519, C5INH-0521, C5INH-0524, C5INH-0525, C5INH-0526, C5INH-0527, C5INH-0532, C5INH-0533, C5INH-0534, C5INH-0535, C5INH-0536, C5INH-0537, C5INH-0538, C5INH-0539, C5INH-0540, C5INH-0541, C5INH-0543, C5INH-0544, C5INH-0545, C5INH-0547, CU0001, CU0002, CU0003, CU0004, CU0005, CU0006, CU0007, CU0008, CU0009, CU0010, CU0011, CU0012, CU0013, CU0014, CU0015, CU0016, CU0017, CU0018, CU0019, CU0020, CU0021, CU0022, CU0023, CU0024, CU0025, CU0026, CU0027, CU0028, CU0029, CU0030, CU0031, CU0032, CU0033, CU0034, CU0035, CU0036, CU0037, CU0038, CU0039, CU0040, CU0041, CU0042, CU0043, CU0044, CU0045, CU0046, CU0047, CU0048, CU0049, CU0050, CU0051, CU0052, CU0053, CU0054, CU0055, CU0056, CU0057, CU0058, CU0059, CU0060, CU0061, CU0062, CU0063, CU0064, CU0065, CU0066, CU0067, CU0100, CU0101, CU0102, CU0103, CU0104, CU0105, CU0106, CU0107, CU0108, CU0109, CU0110, CU0111, CU0112, CU0113, CU0114, CU0115, CU0116, CU0117, CU0118, CU0119, CU0120, CU0121, CU0122, CU0123, CU0124, CU0125, CU0126, CU0127, CU0128, CU0129, CU0130, CU0131, CU0132, CU0133, CU0134, CU0135, CU0136, CU0137, CU0138, CU0139, CU0140, CU0141, CU0142, CU0143, CU0144, CU0145, CU0146, CU0147, CU0148, CU0149, CU0150, CU0151, CU0152, CU0153, CU0154, CU0155, CU0156, CU0157, CU0158, CU0159, CU0160, CU0161, CU0162, CU0163, CU0164, CU0165, CU0166, CU0167, CU0168, CU0169, CU0170, CU0171, CU0172, CU0173, CU0174, CU0175, CU0176, CU0177, CU0178, CU0179, CU0180, CU0181, CU0182, CU0183, CU0184, CU0185, CU0186, CU0187, CU0188, CU0189, CU0190, CU0191, CU0192, CU0193, CU0194, CU0195, CU0196, CU0197, CU0198, CU0199, CU0200, CU0201, CU0202, CU0203, CU0204, CU0205, CU0206, CU0207, CU0208, CU0209, CU0210, CU0211, CU0212, CU0213, CU0214, CU0215, CU0216, CU0217, CU0218, CU0219, CU0220, CU0221, CU0222, CU0223, CU0224, CU0225, CU0226, CU0227, CU0228, CU0229, CU0230, CU0231, CU0232, CU0233, CU0234, CU0235, CU0236, CU0237, CU0238, CU0239, CU0240, CU0241, CU0242, CU0243, CU0244, CU0245, CU0246, CU0247, CU0248, CU0249, CU0250, CU0251, CU0252, CU0253, CU0254, CU0255, CU0256, CU0257, CU0258, CU0259, CU0260, CU0261, CU0262, CU0500, CU0501, CU0502, CU0503, CU0504, CU0505, CU0506, CU0507, CU0508, CU0509, CU0510, CU0511, CU0512, CU0513, CU0514, CU0515, CU0516, CU0517, CU0518, CU0519, CU0520, CU0521, CU0522, CU0523, CU0524, CU0525, CU0526, CU0527, CU0528, CU0529, CU0530, CU0531, CU0532, CU0533, CU0534, CU0535, CU0536, CU0537, CU0538, CU0539, CU0540, CU0541, CU0542, CU0543, CU0544, CU0545, CU0546, CU0547, CU0548, CU0549, CU0550, CU0551, CU0552, CU0553, CU0554, CU0555, CU0556, CU0557, CU0558, CU0559, CU0560, CU0561, CU0562, CU0563, CU0564, CU0565, CU0566, CU0567, CU0568, CU0569, CU0570, CU0571, CU0572, CU0573, CU0574, CU0575, CU0576, CU0577, CU0578, CU0579, CU0580, CU0581, CU0582, CU0583, CU0584, CU0585, CU0586, CU0587, CU0588, CU0589, CU0590, CU0591, CU0592, CU0593, CU0594, CU0595, CU0596, CU0597, CU0598, CU0599, CU0600, CU0601, CU0602, CU0603, CU0604, CU0605, CU0606, CU0607, CU0608, CU0609, CU0610, CU0611, CU0612, CU0613, CU0614, CU0615, CU0616, CU0617, CU0618, CU0619, CU0620, CU0621, CU0622, CU0624, CU0625, CU0626, CU0627, CU0628, CU0629, CU0630, CU0631, CU0632, CU0633, CU0634, CU0635, CU0636, CU0637, CU0638, CU0639, CU0640, CU0641, CU0642, CU0643, CU0644, CU0645, CU0646, CU0647, CU0648, CU0649, CU0650, CU0651, CU0652, CU0653, CU0654, CU0655, CU0656, CU0657, CU0658, CU0659, CU0660, CU0661, CU0662, CU0663, CU0664, CU0665, CU0666, CU0667, CU0668, CU0669, CU0670, CU0671, CU0672, CU0673, CU0674, CU0675, CU0676, CU0677, CU0678, CU0679, CU0680, CU0681, CU0682, CU0683, CU0684, CU0685, CU0686, CU0687, CU0688, CU0689, CU0690, CU0691, CU0692, CU0693, CU0694, CU0695, CU0696, CU0697, CU0698, CU0699, CU0700, CU0701, CU0702, CU0703, CU0704, CU0705, CU0706, CU0707, CU0708, CU0709, CU0710, CU0711, CU0712, CU0713, CU0714, CU0715, CU0716, CU0717, CU0718, CU0719, CU0720, CU0721, CU0722, CU0723, CU0724, CU0725, CU0726, CU0727, CU0728, CU0729, CU0730, CU0731, CU0732, CU0733, CU0734, CU0735, CU0736, CU0737, CU0738, CU0739, CU0740, CU0741, CU0742, CU0743, CU0744, CU0745, CU0746, CU0747, CU0748, CU0749, CU0750, CU0751, CU0752, CU0753, CU0754, CU0755, CU0756, CU0757, CU0758, CU0759, CU0760, CU0761, CU0762, CU0763, CU0764, CU0765, CU0766, CU0767, CU0768, CU0769, CU0770, CU0771, CU0772, CU0773, CU0774, CU0775, CU0776, CU0777, CU0778, CU0779, CU0780, CU0781, CU0782, CU0783, CU0784, CU0785, CU0786, CU0787, CU0788, CU0789, CU0790, CU0791, CU0792, CU0793, CU0794, CU0795, CU0796, CU0797, CU0798, CU0799, CU0800, CU0801, CU0802, CU0803, CU0804, CU0805, CU0806, CU0807, CU0808, CU0809, CU0810, CU0811, CU0812, CU0813, CU0814, CU0815, CU0816, CU0817, CU0818, CU0819, CU0820, CU0821, CU0822, CU0823, CU0824, CU0825, CU0826, CU0827, CU0828, CU0829, CU0830, CU0831, CU0832, CU0833, CU0834, CU0835, CU0836, CU0837, CU0838, CU0839, CU0840, CU0841, CU0842, CU0843, CU0844, CU0845, CU0846, CU0847, SC0001, SC0002, SC0003, SC0004, SC0005, SC0006, SC0007, SC0008, SC0009, SC0010, CU0623, SC0011, SC0012, SC0013, SC0014, SC0015, SC0016, SC0017, SC0018, SC0019, SC0020, SC0021, SC0022, SC0023, SC0024, SC0025, SC0026, SC0027, SC0028, SC0029, SC0030, SC0031, SC0032, SC0033, SC0034, SC0035, SC0036, SC0037, SC0038, SC0039, SC0040, SC0041, SC0042, SC0043, SC0044, SC0045, SC0046, SC0047, SC0048, SC0049, SC0050, SC0051, SC0052, SC0053, SC0054, SC0055, SC0056, SC0057, SC0058, SC0059, SC0060, SC0061, SC0062, SC0063, SC0064, SC0065, SC0066, SC0067, SC0068, SC0069, SC0070, SC0071, SC0072, SC0100, SC0101, SC0102, SC0103, SC0104, SC0105, SC0106, SC0107, SC0108, SC0109, SC0110, SC0111, SC0112, SC0113, SC0114, SC0115, SC0116, SC0117, SC0118, SC0119, SC0120, SC0121, SC0122, SC0123, SC0124, SC0125, SC0126, SC0127, SC0128, SC0129, SC0130, SC0131, SC0132, SC0133, SC0134, SC0135, SC0136, SC0137, SC0138, SC0139, SC0140, SC0141, SC0142, SC0143, SC0144, SC0145, SC0146, SC0147, SC0148, SC0149, SC0150, SC0151, SC0152, SC0153, SC0154, SC0155, SC0156, SC0157, SC0158, SC0159, SC0160, SC0161, SC0162, SC0163, SC0164, SC0165, SC0166, SC0167, SC0168, SC0169, SC0170, SC0171, SC0172, SC0173, SC0174, SC0175, SC0176, SC0177, SC0178, SC0179, SC0180, SC0181, SC0182, SC0183, SC0184, SC0185, SC0186, SC0187, SC0188, SC0189, SC0190, SC0191, SC0192, SC0193, SC0194, SC0195, SC0196, SC0197, SC0198, SC0199, SC0200, SC0201, SC0202, SC0203, SC0204, SC0205, SC0206, SC0207, SC0208, SC0209, SC0210, SC0211, SC0212, SC0213, SC0214, SC0215, SC0216, SC0217, SC0218, SC0219, SC0220, SC0221, SC0222, SC0223, SC0224, SC0225, SC0226, SC0227, SC0228, SC0229, SC0230, SC0231, and SC0232.

28. A pharmaceutical composition comprising:
the compound of claim 1 or a pharmaceutically acceptable salt thereof; and
a pharmaceutically acceptable excipient.

29. (canceled)

31. A method of inhibiting complement activity in a biological system comprising contacting the biological system with a C5 inhibitor, wherein the C5 inhibitor comprises the compound of claim 1.

32-36. (canceled)

37. A method of inhibiting complement activity in a subject, the method comprising administering the compound of claim 1 to the subject.

38. (canceled)

39. A method of treating a complement-related indication in a subject, the method comprising administering the compound of claim 1 to the subject.

40. The method of claim 39, wherein the complement-related indication is selected from the group consisting of paroxysmal nocturnal hemoglobinuria, an inflammatory indication, a wound, an injury, an autoimmune indication, a pulmonary indication, a cardiovascular indication, a neurological indication, a kidney-related indication, an ocular indication, and a pregnancy-related indication.

41-50. (canceled)

51. A compound having a structure of Formula (100): 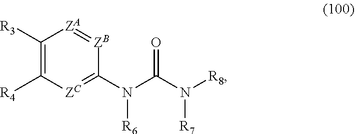
or a pharmaceutically acceptable salt thereof, wherein
Z^{A} is N or CR₂, Z^{B} is N or CR₁, Z^{C} is N or CR₅, provided that
when Z^{A} is N, Z^{B}═CR₁ and Z^{C}═CR₅;
when Z^{B} is N, Z^{A}═CR₂ and Z^{C}═CR₅;
when Z^{C} is N, Z^{A}═CR₂ and Z^{B}═CR₁; and
when both Z^{B} and Z^{C} are N, Z^{A}═CR₂; and
R₁, R₂ and R₈ are H;
R₃ is —OCH₃;
R₄ is an alkoxyl group;
R₆ and R₇, together with the nitrogens to which they are attached and the carbonyl group, forms a 5 to 7-membered heterocycle which is optionally substituted;
R₈ is a substituted alkyl group having the formula 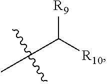
R₉ is hydrogen;
R₁₀ is an optionally substituted aryl or heteroaryl.
